(19) United States
(12) Patent Application Publication
(10) Pub. No.: US 2025/0171459 A1
(43) Pub. Date: May 29, 2025

(54) 2-METHYL-4',5'-DIHYDROSPIRO[PIPERIDINE-4,7'-THIENO[2,3-C]PYRAN] DERIVATIVES AS INHIBITORS OF APOL1 AND METHODS OF USING SAME

(71) Applicant: Vertex Pharmaceuticals Incorporated, Boston, MA (US)

(72) Inventors: Samantha ANGLE, North Cambridge, MA (US); Jingrong CAO, Newton, MA (US); Jon COME, Cambridge, MA (US); Leslie A. DAKIN, Framingham, MA (US); Zachary GALE-DAY, Brookline, MA (US); Elaine B. KRUEGER, Milton, MA (US); Jessica H. OLSEN, Jamaica Plain, MA (US); Timothy J. SENTER, Arlington, MA (US); Akira J. SHIMIZU, Framingham, MA (US); Steven D. STONE, Quincy, MA (US); Tiansheng WANG, Concord, MA (US)

(21) Appl. No.: 18/836,539

(22) PCT Filed: Feb. 8, 2023
(86) PCT No.: PCT/US2023/012618

§ 371 Date: Aug. 7, 2024

Related U.S. Application Data
(60) Provisional application No. 63/307,875, filed on Feb. 8, 2022.

(51) Int. Cl. C07D 495/20; A61K 31/438; A61K 31/444; A61K 31/4545; A61K 31/497; A61K 31/4985; A61K 31/501; A61K 31/506; A61K 31/5365; A61K 31/5377; A61K 31/541; C07D 519/00

(52) U.S. Cl. CPC C07D 495/20; A61K 31/438; A61K 31/444; A61K 31/4545; A61K 31/497; A61K 31/4985; A61K 31/501; A61K 31/506; A61K 31/5365; A61K 31/5377; A61K 31/541; C07D 519/00

(57) ABSTRACT

The disclosure provides at least one compound, tautomer, deuterated derivative, or pharmaceutically acceptable salt chosen from compounds of Formula I, tautomers thereof, deuterated derivatives of those compounds or tautomers, and pharmaceutically acceptable salts of any of the foregoing, compositions comprising the same, and methods of using the same, including uses in treating APOL1-mediated diseases, including pancreatic cancer, focal segmental glomerulosclerosis (FSGS), and/or non-diabetic kidney disease (NDKD).

This application claims the benefit of U.S. Provisional Application No. 63/307,875, filed on Feb. 8, 2022, the contents of which are incorporated by reference in its entirety.

This disclosure provides compounds that may inhibit apolipoprotein L1 (APOL1) and methods of using those compounds to treat APOL1-mediated diseases, such as, e.g., pancreatic cancer, focal segmental glomerulosclerosis (FSGS), and/or non-diabetic kidney disease (NDKD). In some embodiments, the FSGS and/or NDKD is associated with at least one of the 2 common APOL1 genetic variants (G1: S342G:I384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV)_associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed G1 and G2, which are referred to as the “APOL1 risk alleles.” G1 encodes a correlated pair of non-synonymous amino acid changes (S342G and 1384M), G2 encodes a 2 amino acid deletion (N388del:Y389del) near the C terminus of the protein, and G0 is the ancestral (low risk) allele. A distinct phenotype of NDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no or just 1 APOL1 genetic risk variant. Alternatively in AMKD, higher levels of proteinuria and accelerated loss of kidney function can also occur in patients with one risk allele. See, G. Vajgel et al., J. Rheumatol., November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against Trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing end-stage kidney disease (ESKD) and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

One aspect of the disclosure provides at least one compound, tautomer, deuterated derivative, or pharmaceutically acceptable salt chosen from compounds of Formulae I, tautomers of Formula I, deuterated derivatives of those compounds or tautomers, and pharmaceutically acceptable salts of any of the foregoing, which can be employed in the treatment of diseases mediated by APOL1, such as FSGS and NDKD. For example, in some embodiments, the at least one compound is a compound represented by Formula I:

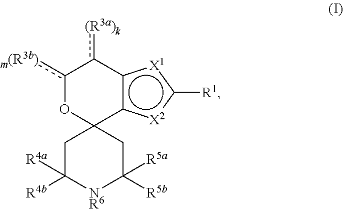

a tautomer thereof, a deuterated derivative of that compound or tautomer, or a pharmaceutically acceptable salt of any of the foregoing, wherein:
X¹ and X² are chosen from —S—, —S(═O)₂—, —S(═O)—, and —CR², wherein:
   one of X¹ and X² is chosen from —S—, —S(═O)₂—, and —S(═O)—;
   when X¹ is —S—, —S(═O)₂—, or —S(═O)—, then X² is —CR²; and
   when X² is —S—, —S(═O)₂—, or —S(═O)—, then X¹ is —CR²;
R¹ is chosen from cyano, halogen, C₁-C₄ alkyl, C₁-C₄ haloalkyl, and C₃-C₆ cycloalkyl groups, wherein:
   the C₁-C₄ alkyl of R¹ is optionally substituted with 1 to 3 groups independently chosen from —OH and C₁-C₄ alkoxy groups;
R² is chosen from hydrogen, C₁-C₆ alkyl, —C(═O)O(C₁-C₄ alkyl), —C(═O)NRⁿR^{o}, and halogen groups, wherein:
   the C₁-C₆ alkyl of R² is optionally substituted with 1 to 3 groups independently chosen from —OH, halogen, and C₁-C₄ alkoxy groups; and
   Rⁿ and R^{o} are independently chosen from hydrogen, C₁-C₄ alkyl, C₁-C₄ haloalkyl, C₃-C₆ cycloalkyl, and —(C₁-C₄ alkylene)R^{p} groups, wherein R^{p} is chosen from C₃-C₆ cycloalkyl groups; or
R¹ and R², together with the carbon atoms to which they are attached, form a C₆ aryl group; each R^{3a} is independently chosen from —OH, —CN, —NR^{a1}R^{a2}, C₁-C₆ alkyl, C₁-C₆ haloalkyl, C₁-C₆ alkoxy, —OC(═O)(C₁-C₄ alkyl), 6- to 8-membered aryl, 6- to 8-membered heteroaryl, and halogen groups, wherein:
   each R^{a1} and R^{a2} is independently chosen from hydrogen, C₁-C₄ alkyl, and —C(═O)(C₁-C₄ alkyl) groups; or
two R^{3a} taken together form an oxo group; or
two R^{3a}, together with the carbon atom to which they are attached, form a C₃-C₆ cycloalkyl group;
each R^{3b} is independently chosen from C₁-C₄ alkyl groups, wherein:
   the C₁-C₄ alkyl of R^{3b} is optionally substituted with 1 to 3 groups independently chosen from —OH, halogen, and C₁-C₄ alkoxy groups; or
one R^{3a} and one R^{3b}, together with the carbon atoms to which they are attached, form a C₃-C₆ cycloalkyl group;
k is chosen from 0, 1, and 2;
m is chosen from 0, 1, and 2;
R^{4a}, R^{4b}, R^{5a}, and R^{5b} are each independently chosen from hydrogen and C₁-C₄ alkyl groups;
R⁶ is chosen from C₁-C₆ alkyl, —C(═O)O(C₁-C₄ alkyl), and

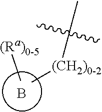

groups, wherein:
   the C₁-C₆ alkyl of R⁶ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
      the C₆ aryl and —O—(C₆ aryl) groups are each optionally substituted with 1 to 3 groups independently chosen from halogen and C₁-C₄ haloalkyl groups;
   Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
      R^{a}, for each occurrence, is independently chosen from halogen, cyano, C₁-C₈ alkyl, C₁-C₆ haloalkyl, C₂-C₈ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ, —C(═O)OR^{k}, —C(═O)(C₁-C₄ alkylene)OR^{k},
   —C(═O)R^{k}, —C(═O)(C₁-C₄ alkylene)S(═O)ₚR^{k}, —C(═O)(C₁-C₄ alkylene)S(═O)ₚNR^{h}Rⁱ,
   —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k},—NR^{h}Rⁱ, —NH(CH₂)_{q}CHR^{h}Rⁱ, —NH(CH₂)_{q}NR^{h}Rⁱ, —NR^{h}C(═O)R^{k},
   —NR^{h}C(═O)OR^{k}, —NR^{h}C(═O)(C₁-C₄ alkylene)OR^{k}, —NR^{h}C(═O)O(C₁-C₄ alkylene)R^{k},
   —NR^{h}C(═O)NRⁱR^{j}, —NR^{h}C(═O)(C₁-C₄ alkylene)NRⁱS(═O)ₚR^{k}, —NR^{h}S(═O)ₚR^{k}, —NR^{h}C(═O)(C₁-C₄ alkylene)S(═O)ₚR^{k}, —NR^{h}S(═O)ₚ(C₁-C₄ alkylene)C(═O)OR^{k}. —NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —NR^{h}C(═O)(C₁-C₆ alkylene)[O(CH₂)_{q}]ᵣOC(═O)—NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
      the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)-NRⁱS(═O)ₚR^{k}, —C(═O)(C₁-C₄ alkylene)-NR^{h}C(═O)R^{k}, —NR^{h}C(═O)O(C₁-C₄ alkylene)R^{k}, —NR^{h}C(═O)(C₁-C₄ alkylene)-OR^{k}, NR^{h}S(═O)ₚ(C₁-C₄ alkylene)C(═O)OR^{k}, and —NR^{h}C(═O)(C₁-C₄ alkylene)NRⁱS(═O)ₚR^{k} is optionally substituted with 1 to 3 —OH groups,
      the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ,
      —NR^{h}C(═O)R^{k}, —NR^{h}C(═O)OR^{k}, —NR^{h}C(═O)NRⁱR^{j}, —NR^{h}S(═O)R^{k}, —OR^{k},
      [O(CH₂)_{q}]ᵣOH, —OC(═O)R^{k}, —OC(═O)OR^{k}, —OC(═O)NR^{h}Rⁱ, —SR^{k}, —S(═O)ₚR^{k},
      —S(═O)ₚNR^{h}Rⁱ, —[O(CH₂)_{q}]ᵣO(C₁-C₄ alkyl), —O—(C₆ aryl or 5-to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups; and
      the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, and 5- to 10-membered heterocyclyl groups, wherein:
         R^{h}, Rⁱ, and R^{j}, for each occurrence, are each independently chosen from hydrogen, C₁-C₆ alkyl, C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
         the C₁-C₆ alkyl of any one of R^{h}, Rⁱ, and R^{j} is optionally substituted with 1 to 4 groups independently chosen from halogen, cyano, —OH, C₁-C₄ alkoxy, —C(═O)NH(C₁-C₄ alkyl), 5- to 10-membered heteroaryl (optionally substituted with 1 to 3 R^{m} groups), and 5- to 10-membered heterocyclyl (optionally substituted with 1 to 3 R^{m} groups) groups;
         R^{k}, for each occurrence, is independently chosen from hydrogen, NH₂, (optionally substituted with 1 or 2 groups chosen from C₁-C₃ alkyl), C₁-C₆ alkyl, benzyl, C₆ aryl, C₃-C₆ carbocyclyl, 5- to 10-membered heterocyclyl, and 5- to 10-membered heteroaryl groups, wherein:
         the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 4- to 10-membered heterocyclyl (optionally substituted with 1 to 3 —OH groups), 5- to 10-membered aryl (optionally substituted with 1 to 3 groups chosen from C₁-C₄ alkyl and halogen), and 5- to-10-membered heteroaryl (optionally substituted with 1 to 3 —OH groups) groups; and
         the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₄ alkyl (optionally substituted by 1 to 3 —OH groups), C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups,
         the 5- to 10-membered heteroaryl and 5- to 10-membered heterocyclyl of any one of R^{k} are each optionally substituted with 1 to 3 groups independently chosen from halogen, oxo, cyano, —C(═O)CH₃, —NH₂, —OH, C₁-C₄ alkyl (optionally substituted by 1 to 3 —OH groups), C₁-C₄ haloalkyl, 5- to 10-membered heterocyclyl, and C₁-C₄ alkoxy groups;
         R^{m}, for each occurrence, is independently chosen from halogen, cyano, oxo, —NH₂, C₁-C₆ alkyl, C₁-C₆ alkoxy, —C(═O)R^{k}, —S(═O)ₚR^{k}, —OR^{k}, and 5- to 10-membered heterocyclyl groups, wherein:
         the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
   p, for each occurrence, is an integer independently chosen from 1 and 2; and
   q and r, for each occurrence, is an integer independently chosen from 0, 1, 2, and 3.

In some embodiments, at least one compound, tautomer, deuterated derivative, or pharmaceutically acceptable salt of the disclosure is a compound represented by the structural Formulae Ia, Ib, Ic, Id, Ie, If, Ig, Ih, Ii, Ij, Ik, Il, and Im, as follows:

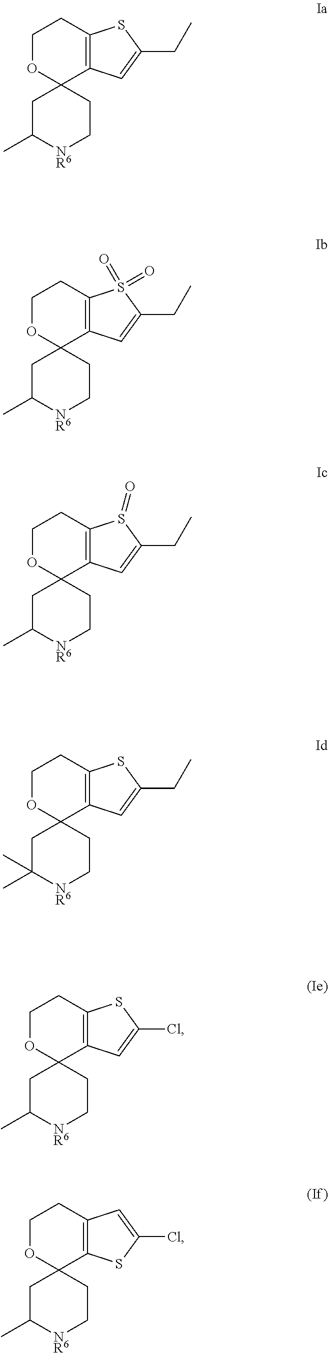

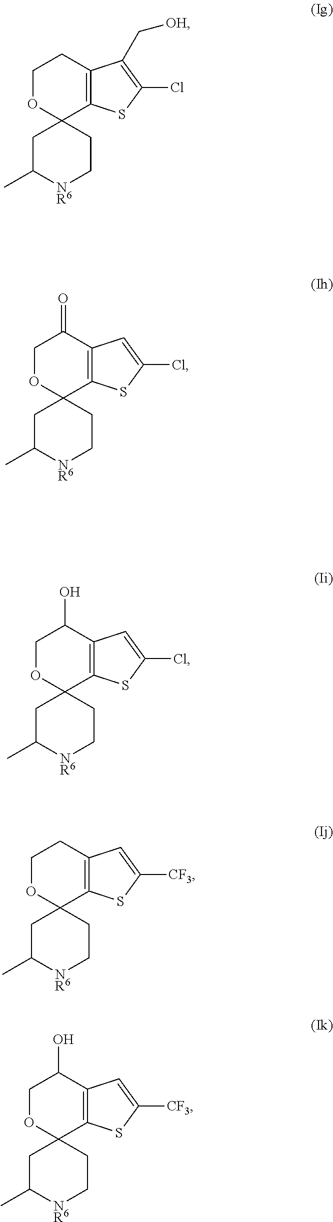

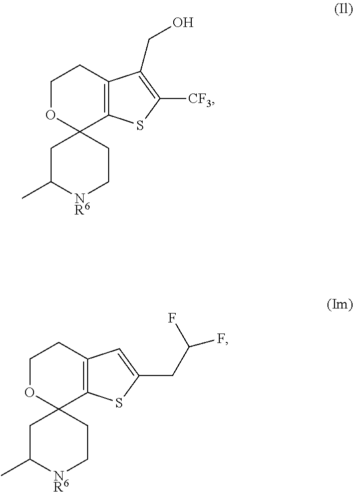

wherein R⁶ is as defined above for Formula I.

In some embodiments, in the compound, tautomer, deuterated derivative, or pharmaceutically acceptable salt of Formulae I, Ia, Ib, Ic, Id, Ie, If, Ig, Ih, Ii, Ij, Ik, Il, and Im, R⁶ in the is chosen from:

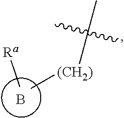

wherein Ring B is a 5-membered heteroaryl, and R^{a} is as defined for Formula I.

In some embodiments, in the compound, tautomer, deuterated derivative, or pharmaceutically acceptable salt of Formulae I, Ia, Ib, Ic, Id, Ie, If, Ig, Ih, Ii, Ij, Ik, Il, and Im, R⁶ is chosen from:

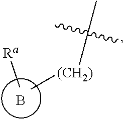

wherein Ring B is a 5-membered heteroaryl, and R^{a} is oxo or is chosen from C₁-C₈ alkyl, C₃-C₁₂ carbocyclyl and C₆ and C₁₀ aryl, each of which may be optionally substituted with 1 to 3 groups chosen from halogen and C₁-C₈ alkyl (wherein the C₁-C₈ alkyl may be optionally substituted with 1 to 3 groups chosen from halogen, —OH, SO₂CH₃, and SO₂NH₂).

In some embodiments, in the compound, tautomer, deuterated derivative, or pharmaceutically acceptable salt of Formulae I, Ia, Ib, Ic, Id, Ie, If, Ig, Ih, Ii, Ij, Ik, Il, and Im, R⁶ is chosen from:

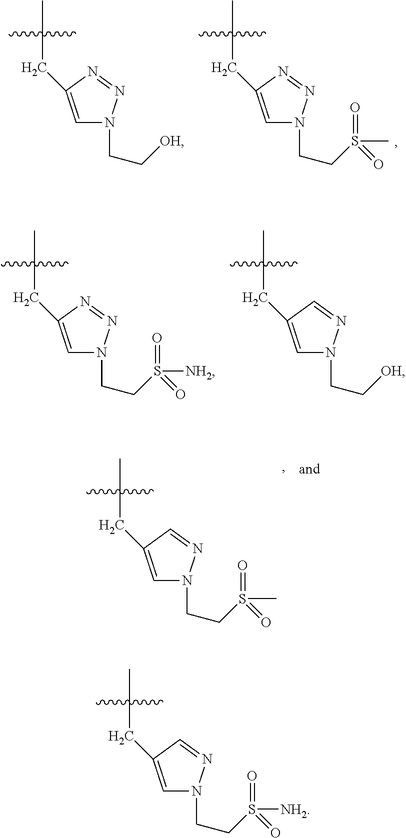 [SMILES: *Cc1cn(CCO)nn1 ; *Cc1cn(CCS(C)(=O)=O)nn1 ; *Cc1cn(CCS(N)(=O)=O)nn1 ; *Cc1cnn(CCO)c1 ; *Cc1cnn(CCS(C)(=O)=O)c1 ; *Cc1cnn(CCS(N)(=O)=O)c1]

In one aspect of the disclosure, the compounds of Formulae I, Ia, Ib, Ic, Id, Ie, If, Ig, Ih, Ii, Ij, Ik, Il, and Im are chosen from Compounds 1 to 1183, tautomers thereof, deuterated derivatives of those compounds and tautomers and pharmaceutically acceptable salts of any of the foregoing. In some embodiments, the disclosure provides a pharmaceutical composition comprising at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing. In some embodiments, the pharmaceutical composition may comprise at least one compound chosen from Compounds 1 to 1183, tautomers thereof, deuterated derivatives of those compounds or tautomers, and pharmaceutically acceptable salts of any of the foregoing. These compositions may further include at least one additional active pharmaceutical ingredient and/or at least one carrier.

Another aspect of the disclosure provides methods of treating an APOL1-mediated disease comprising administering to a subject in need thereof, at least one compound, tautomer, deuterated derivative, or pharmaceutically acceptable salt chosen from compounds of Formulae Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1-1183, tautomers thereof, deuterated derivatives of those compounds or tautomers, and pharmaceutically acceptable salts of any of the foregoing.

Another aspect of the disclosure provides methods of treating an APOL1-mediated cancer (such as, e.g., pancreatic cancer) comprising administering to a subject in need thereof, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1-1183, tautomers thereof, deuterated derivatives of those compounds or tautomers, and pharmaceutically acceptable salts of any of the foregoing.

Another aspect of the disclosure provides methods of treating APOL1-mediated kidney disease (such as, e.g., ESKD, FSGS and/or NDKD) comprising administering to a subject in need thereof, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1-1183, tautomers thereof, deuterated derivatives of those compounds or tautomers, and pharmaceutically acceptable salts of any of the foregoing.

In some embodiments, the methods of treatment include administration of at least one additional active agent to the subject in need thereof, either in the same pharmaceutical composition as the at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, or as separate compositions. In some embodiments, the methods comprise administering at least one compound, tautomer, deuterated derivative, or pharmaceutically acceptable salt chosen from Compounds 1-1183, tautomers thereof, deuterated derivatives of those compounds or tautomers, and pharmaceutically acceptable salts of any of the foregoing with at least one additional active agent, either in the same pharmaceutical composition or in a separate composition.

Also provided are methods of inhibiting APOL1, comprising administering to a subject in need thereof, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods of inhibiting APOL1 comprise administering at least one compound, tautomer, deuterated derivative, or pharmaceutically acceptable salt chosen from Compounds 1-1183, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt.

All of Compounds 1 to 1183 disclosed herein have demonstrated the ability to inhibit ApoL1 in one or more assays. In some embodiments, a compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, exclude Compound 285, Compound 489, Compound 539, Compound 691, Compound 692, Compound 741, Compound 747, Compound 749, Compound 751, Compound 752, Compound 753, Compound 795, Compound 814, and Compound 868.

### DETAILED DESCRIPTION

### Definitions

The term “APOL1,” as used herein, means apolipoprotein L1 protein and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function, and associated with 2 common APOL1 genetic variants (G1: S342G:I384M and G2: N388del:Y389del).

The term “NDKD,” as used herein, means non-diabetic kidney disease, which is characterized by severe hypertension and progressive decline in kidney function, and associated with 2 common APOL1 genetic variants (G1: S342G:I384M and G2: N388del:Y389del).

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in total will be less than 49.9% of the compound. In other embodiments, the relative amount of such isotopologues in total will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a reference compound only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C₁, are within the scope of this disclosure.

Unless otherwise indicated, structures depicted herein are also meant to include all isomeric forms of the structures, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor,” as used herein, means the ratio between the isotopic abundance and the natural abundance of a specified isotope.

The term “alkyl” or “aliphatic,” as used herein, means a straight-chain (i.e., linear or unbranched) or branched, substituted or unsubstituted hydrocarbon chain that is completely saturated. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms. In some embodiments, alkyl groups contain 1 to 4 alkyl carbon atoms, in other embodiments, alkyl groups contain 1 to 3 alkyl carbon atoms, and in yet other embodiments, alkyl groups contain 1 or 2 alkyl carbon atoms. In some embodiments, alkyl groups are linear or straight-chain or unbranched. In some embodiments, alkyl groups are branched.

The terms “carbocyclyl,” “cycloalkyl” and “cyclic alkyl,” as used herein, refer to a monocyclic C₃_hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The terms “carbocyclyl” or “cycloaliphatic,” as used herein, encompass the terms “cycloalkyl” or “cyclic alkyl,” and refer to a monocyclic C₃_₈ hydrocarbon or a spirocyclic, fused, or bridged bicyclic or tricyclic C₈₋₁₄ hydrocarbon that is completely saturated, or is partially saturated as in it contains one or more units of unsaturation but is not aromatic, wherein any individual ring in said bicyclic ring system has 3 to 7 members. Bicyclic carbocyclyls include combinations of a monocyclic carbocyclic ring fused to a phenyl. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl.

The term “heteroalkyl,” or “heteroaliphatic,” as used herein, means an alkyl or aliphatic group as defined above, wherein one or two carbon atoms are independently replaced by one or more of oxygen, sulfur, nitrogen, phosphorus, or silicon.

The term “alkenyl,” as used herein, means a straight-chain (i.e., linear or unbranched) or branched hydrocarbon chain that contains one or more double bonds. In some embodiments, alkenyl groups are straight-chain. In some embodiments, alkenyl groups are branched.

The terms “heterocycle,” “heterocyclyl,” and “heterocyclic,” are used herein interchangeably to refer to non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl.

In some embodiments, the heterocycle comprises a ring atom substituted with one or more oxo groups (such as, e.g., a C═O group, a S═O group, or a SO₂ group).

In some embodiments, the “heterocycle,” “heterocyclyl,” “heterocycloaliphatic,” or “heterocyclic” group has 3 to 14 ring members in which one or more ring members is a heteroatom independently chosen from oxygen, sulfur, nitrogen, silicon, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, silicon, and phosphorus, the quaternized form of any basic nitrogen or; a substitutable nitrogen of a heterocyclic ring, for example, N (as in 3,4-dihydro-2H-pyrrolyl), NH (as in pyrrolidinyl) or NR⁺ (as in N-substituted pyrrolidinyl)). In some embodiments, the heterocycle has one heteroatom that is a nitrogen atom. In some embodiments, the heterocycle has one heteroatom that is an oxygen atom. In some embodiments, the heterocycle has two heteroatoms that are each independently chosen from nitrogen and oxygen. In some embodiments, the heterocycle has three heteroatoms that are each independently chosen from nitrogen and oxygen. In some embodiments, the heterocyclyl is a 3- to 12-membered heterocyclyl. In some embodiments, the heterocyclyl is a 3- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. Non-limiting examples of monocyclic heterocyclyls include piperidinyl, piperazinyl, tetrahydropyranyl, azetidinyl, tetrahydrothiophenyl 1,1-dioxide, and the like.

The term “unsaturated,” as used herein, means that a moiety has one or more units or degrees of unsaturation. Unsaturation is the state in which not all of the available valence bonds in a compound are satisfied by substituents and thus the compound contains double or triple bonds.

The term “alkoxy” or “thioalkyl,” as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl,” “haloalkenyl,” and “haloalkoxy,” as used herein, mean a linear or branched alkyl, alkenyl, or alkoxy, respectively, which is substituted with one or more halogen atoms. Non-limiting examples of haloalkyl groups include —CHF₂, —CH₂F, —CF₃, —CF₂—, and perhaloalkyls, such as —CF₂CF₃. Non-limiting examples of haloalkoxy groups include —OCHF₂, —OCH₂F, —OCF₃, and —OCF₂.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2]p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Non-limiting examples of aryl groups include phenyl (C₆) and naphthyl (C₁₀) rings.

The term “heteroaryl,” used alone or as part of a larger moiety as in “heteroarylalkyl” or “heteroarylalkoxy,” refers to monocyclic or spirocyclic, fused, or bridged bicyclic or tricyclic ring systems having a total of five to fourteen ring members, wherein at least one ring in the system is aromatic, wherein at least one ring in the system contains one or more heteroatoms, and wherein each ring in a bicyclic or tricyclic ring system contains 3 to 7 ring members. Bicyclic heteroaryls include the following combinations of monocyclic rings: a monocyclic heteroaryl fused to another monocyclic heteroaryl; and a monocyclic heteroaryl fused to a phenyl. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

In some embodiments, the heteroaryl comprises a ring atom substituted with one or more oxo groups (such as, e.g., a C═O group, a S═O group, or a SO₂ group). Illustratively, a non-limiting example of a heteroaryl group is a benzo[d]oxazol-2(3H)-one group.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂C₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably herein and refer to an animal, including a human.

The terms “effective dose” and “effective amount” are used interchangeably herein and refer to that amount of compound that produces a desired effect for which it is administered (e.g., improvement in a symptom of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to, the following: complete or partial remission, lower risk of kidney failure (e.g., ESRD), and disease-related complications (e.g., edema, susceptibility to infections, or thrombo-embolic events). Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salts of any of the foregoing, may be administered once daily, twice daily, or three times daily, for example, for the treatment of AMKD, including FSGS and/or NDKD. In some embodiments, at least one compound chosen from Compounds 1 to 1183, tautomers thereof, deuterated derivatives of those compounds and tautomers, and pharmaceutically acceptable salts of any of the foregoing may be administered once daily, twice daily, or three times daily, for example, for the treatment of AMKD, including FSGS and/or NDKD. In some embodiments, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salts of any of the foregoing is administered once daily. In some embodiments, at least one compound chosen from Compounds 1 to 1183, tautomers thereof, deuterated derivatives of those compounds and tautomers, and pharmaceutically acceptable salts of any of the foregoing is administered once daily. In some embodiments, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salts of any of the foregoing is administered twice daily. In some embodiments, at least one compound chosen from Compounds 1 to 1183, tautomers thereof, deuterated derivatives of those compounds and tautomers, and pharmaceutically acceptable salts of any of the foregoing is administered twice daily. In some embodiments, at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salts of any of the foregoing is administered three times daily. In some embodiments, at least one compound chosen from Compounds 1 to 1183, tautomers thereof, deuterated derivatives of those compounds and tautomers, and pharmaceutically acceptable salts of any of the foregoing is administered three times daily.

In some embodiments, 2 mg to 1500 mg or 5 mg to 1000 mg of at least one compound chosen from Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing is administered once daily, twice daily, or three times daily. In some embodiments, 2 mg to 1500 mg or 5 mg to 1000 mg of at least one compound chosen from Compounds 1 to 1183, tautomera thereof, deuterated derivative of those compounds and tautomers, and pharmaceutically acceptable salts of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound or pharmaceutically acceptable salt chosen from compounds of Formula I and pharmaceutically acceptable salts thereof” includes 1000 mg of a compound of Formula I and a concentration of a pharmaceutically acceptable salt of compounds of Formula I equivalent to 1000 mg of a compound of Formula I.

As used herein, the term “ambient conditions” means room temperature, open air condition, and uncontrolled humidity condition.

### Compounds and Compositions

In some embodiments, at least one compound chosen from Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salt of any of the foregoing may be employed in the treatment of AMKD, including FSGS and NDKD. In some embodiments, the compound of Formulae I, Ia, Ib, Ic, Id, Ie, If, Ig, Ih, Ii, Ij, Ik, Il, and Im, may be chosen from Compounds 1 to 1183, tautomers thereof, deuterated derivatives of those compounds and tautomers, and pharmaceutically acceptable salts of any of the foregoing. In some embodiments, a pharmaceutical composition comprising at least one compound chosen from Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salt of any of the foregoing, may be employed in the treatment of AMKD, including FSGS and NDKD. In some embodiments the pharmaceutical composition comprises at least one compound chosen from Compounds 1 to 1183, tautomers thereof, deuterated derivatives of those compounds and tautomers, and pharmaceutically acceptable salt of any of the foregoing.

In some embodiments of Formula I:

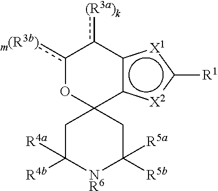

the variable X¹ is chosen from S, S(═O), and S(═O)₂ and the variable X² is —CR². In some embodiments of Formula I, the variable X¹ is S, and the variable X² is —CR². In some embodiments of Formula I, the variable X² is chosen from S, S(═O), and S(═O)₂ and the variable X¹ is —CR². In some embodiments of Formula I, the variable X² is S, and the variable X¹ is —CR².

In some embodiments (including the embodiments discussed above that define variables X¹ and X²), R² is chosen from hydrogen, C₁-C₆ alkyl (optionally substituted with 1 to 3 groups independently chosen from —OH, halogen, and C₁-C₄ alkoxy groups), —C(═O)O(C₁-C₄ alkyl), —C(═O)NRⁿR^{o}, and halogen groups, wherein Rⁿ and R^{o} are independently chosen from hydrogen, C₁-C₄ alkyl, C₁-C₄ haloalkyl, C₃-C₆ cycloalkyl, and —(C₁-C₄ alkylene)R^{p} groups, and wherein R^{p} is chosen from C₃-C₆ cycloalkyl groups.

In some embodiments (including the embodiments discussed above that define variables X¹ and X²), R² is chosen from hydrogen, halogen, —CH₂OH, —CH₂(OH)CH₃, and —C(═O)NRⁿR^{o}. In some embodiments, R² is hydrogen. In some embodiments (including the embodiments discussed above that define variables X¹ and X²), R² is halogen. In some embodiments (including the embodiments discussed above that define variables X¹ and X²), R² is Br. In some embodiments, R² is Cl. In some embodiments (including the embodiments discussed above that define variables X¹ and X²), R² is CH₃. In some embodiments (including the embodiments discussed above that define variables X¹ and X²), R² is —CH₂OH. In some embodiments, R² is CH₂(OH)CH₃. In some embodiments, R² is —C(═O)OCH₃.

In some embodiments (including the embodiments discussed above that define variables X¹ and X²), R² is —C(═O)NRⁿR^{o}, and halogen groups, wherein Rⁿ and R^{o} are independently chosen from hydrogen, C₁-C₄ alkyl, C₁-C₄ haloalkyl, C₃-C₆ cycloalkyl, and —(C₁-C₄ alkylene)R^{p} groups, and wherein R^{p} is chosen from C₃-C₆ cycloalkyl groups. In some embodiments (including the embodiments discussed above that define variables X¹ and X²), R² is —C(═O)NRⁿR^{o} (wherein Rⁿ is hydrogen and R^{o} is CH₃). In some embodiments (including the embodiments discussed above that define variables X¹ and X²), R² is —C(═O)NRⁿR^{o} (wherein Rⁿ is hydrogen and R^{o} is CH₃ substituted with a cyclopropyl group). In some embodiments (including the embodiments discussed above that define variables X¹ and X²), R² is

—C(═O)NH CH₂CH₂CH₃ (i.e., wherein Rⁿ is hydrogen and R^{o} is —CH₂CH₂CH₃). In some embodiments (including the embodiments discussed above that define variables X¹ and X²), R² is —C(═O)NH CH₂CF₂ (i.e., wherein Rⁿ is hydrogen and R^{o} is —CH₂CF₂).

In some embodiments of Formula I (including the embodiments set forth above defining variables X¹, X², and R²), the variable R¹ is chosen from cyano, halogen, —C₁-C₄ alkyl, —C₁-C₄ haloalkyl, and —C₃-C₆ cycloalkyl groups, wherein: the —C₁-C₄ alkyl of R¹ is optionally substituted with 1 to 3 groups independently chosen from —OH and —C₁-C₄ alkoxy groups.

In some embodiments of Formula I (including the embodiments set forth above defining variables X¹, X², and R²), the variable R¹ is chosen from —CN, —Br, —Cl, —C₁-C₄ alkyl, —CH₂OH, —CH₂CF₂, —CF₂CF₂, —CF₂, —CF₃, —CH₂OCH₃, —CH₂OCH₂CH₃, cyclopropyl, and cyclobutyl. In some embodiments of Formula I (including the embodiments set forth above defining variables X¹, X², and R²), the variable R¹ is chosen —CH₃, —CH₂OH, —CH₂CH₃, —CH₂CH₂CH₃, tert-butyl, —Cl, —CH₂—CF₂, —CF₂CF₃, and —CF₃. In some embodiments of Formula I (including the embodiments set forth above defining variables X¹, X², and R²), the variable R¹ is deuterated, e.g. —CDOCH₂CH₃.

In some embodiments of Formula I (including the embodiments set forth above defining variables X¹, X², and R²), the variable R¹ is CF₃. In some embodiments of Formula I (including the embodiments set forth above defining variables X¹, X², and R²), the variable R¹ is Cl. In some embodiments of Formula I (including the embodiments set forth above defining variables X¹, X², and R²), the variable R¹ is —CH₂OH. In some embodiments of Formula I (including the embodiments set forth above defining variables X¹, X², and R²), the variable R¹ is —CH₂CF₃. In some embodiments of Formula I (including the embodiments set forth above defining variables X¹, X², and R²), the variable R¹ is —CF₂CF₃. In some embodiments of Formula I (including the embodiments set forth above defining variables X¹, X², and R²), the variable R¹ is —CH₂(OH)CH₃.

In some embodiments of Formula I (including the embodiments set forth above defining variables X¹ and X²), variables R¹ and R², together with the carbon atoms to which they are attached, form a C₆ aryl group.

In some embodiments of Formula I (including the embodiments set forth above defining variables X¹ and X²), the variable R¹ is —Cl and the variable R² is —CH₂OH. In some embodiments of Formula I (including the embodiments set forth above defining variables X¹ and X²), the variable R¹ is Cl and the variable R² is —CH₃. In some embodiments of Formula I (including the embodiments set forth above defining variables X¹ and X²), the variable R¹ is Cl and the variable R² is hydrogen. In some embodiments of Formula I (including the embodiments set forth above defining variables X¹ and X²), the variable R¹ is —Cl and the variable R² is —Cl.

In some embodiments of Formula I (including the embodiments set forth above defining variables X¹ and X²), the variable R¹ is —CF₃ and the variable R² is hydrogen. In some embodiments of Formula I (including the embodiments set forth above defining variables X¹ and X²), the variable R¹ is —CF₂CF₃ and the variable R² is hydrogen. In some embodiments of Formula I (including the embodiments set forth above defining variables X¹ and X²), the variable R¹ is —CH₂CF₃ and the variable R² is hydrogen. In some embodiments of Formula I (including the embodiments set forth above defining variables X¹ and X²), the variable R¹ is —CF₂ and the variable R² is —CH₂OH.

In some embodiments of Formula I (including the embodiments set forth above defining variables X¹ and X²), the variable R¹ is —CF₃ and the variable R² is —CH₂OH. In some embodiments of Formula I (including the embodiments set forth above defining variables X¹ and X²), the variable R¹ is —CF₃ and the variable R² is —CH₂(OH)CH₃. In some embodiments of Formula I (including the embodiments set forth above defining variables X¹ and X²), the variable R¹ is —CF₃ and the variable R² is —Cl.

In some embodiments of Formula I (including the embodiments set forth above defining variables X¹ and X²), the variable R¹ is CH₃ and the variable R² is hydrogen. In some embodiments of Formula I (including the embodiments set forth above defining variables X¹ and X²), the variable R¹ is CH₂CH₃ and the variable R² is hydrogen. In some embodiments of Formula I (including the embodiments set forth above defining variables X¹ and X²), the variable R¹ is CH₂OH and the variable R² is hydrogen.

In some embodiments of Formula I, the variables m and k are both zero, resulting in the absence of variables R^{3a} and R^{3b}. In some embodiments of Formula I, the variable m is zero, resulting in the absence of variable R^{3b}, and the variable k is one, resulting in the presence of one R^{3a} variable. In some embodiments of Formula I, the variable m is zero, resulting in the absence of variable R^{3b}, and the variable k is two, resulting in the presence of two R^{3a} variables in Formula I.

In some embodiments of Formula I, (including the embodiments set forth above defining variables X¹ and X², R¹, R²), each R^{3a} is independently chosen from —OH, —CN, —NR^{a1}R^{a2}, C₁-C₆ alkyl, C₁-C₆ haloalkyl, C₁-C₆ alkoxy, —OC(═O)(C₁-C₄ alkyl), 6- to 8-membered aryl, 6- to 8-membered heteroaryl, and halogen groups, wherein:
each R^{a1} and R^{a2} is independently chosen from hydrogen, C₁-C₄ alkyl, and —C(═O)(C₁-C₄ alkyl) groups; or
   two R^{3a} taken together form an oxo group; or
   two R^{3a}, together with the carbon atom to which they are attached, form a C₃-C₆ cycloalkyl group.

In some embodiments of Formula I, (including the embodiments set forth above defining variables X¹ and X², R¹, R²), each R^{3a} is chosen from —OH, F, F₂, CF₂, —OCH₃, —OCH₂CH₃, —OCH₂(CH₃)₂,

—OC(═O)CH₃, NH₂, NHC(═O)CH₃, CN,

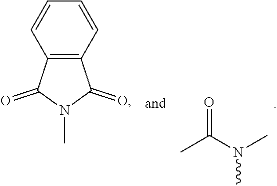

In some embodiments of Formula I, the variable k is one, and variable R^{3a} is chosen from —OH, F, F₂, CF₂, —OCH₃, —OCH₂CH₃, —O—CH₂(CH₃)₂, —O—C(═O)CH₃, NH₂, NH—C(═O)CH₃, CN,

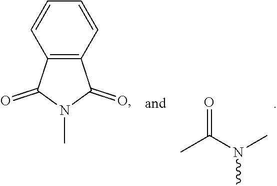

In some embodiments of Formula I (including the embodiments set forth above defining variables X¹ and X², R¹, R²), variable m is zero, variable k is one, and variable R^{3a} is chosen from —OH, F, F₂, CF₂, —OCH₃, —OCH₂CH₃, —O—CH₂(CH₃)₂, —O—C(═O)CH₃, NH₂, NH—C(═O)CH₃, CN,

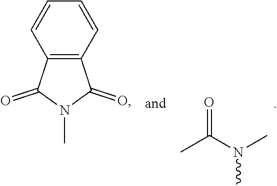

In some embodiments of Formula I (including the embodiments set forth above defining variables X¹ and X², R¹, R²), variable m is zero, variable k is one, and variable R^{3a} is —OH. In some embodiments of Formula I (including the embodiments set forth above defining variables X¹ and X², R¹, R²), variable m is zero, variable k is one, and variable R^{3a} is chosen from —OCH₃ and —OCH₂CH₃.

In some embodiments of Formula I (including the embodiments set forth above defining variables X¹ and X², R¹, R²), variable k is two, and the two R^{3a} variables are chosen from (a) —CF₂ and —OH, (b) —CH₃ and —OH, and (c) —OH and phenyl. In some embodiments of Formula I (including the embodiments set forth above defining variables X¹ and X², R¹, R²), variable m is zero, variable k is two, and the two R^{3a} variables are chosen from (a) —CF₂ and —OH, (b) —CH₃ and —OH, and (c) —OH and phenyl.

In some embodiments of Formula I (including the embodiments set forth above defining variables X¹ and X², R¹, R²), variable R^{3a} is deuterated. In some embodiments, the deuterated R^{3a} is

### —O—CD₃.

In some embodiments of Formula I (including the embodiments set forth above defining variables X¹ and X², R¹, R²), two R^{3a} are taken together to form an oxo group.

In some embodiments of Formula I (including the embodiments set forth above defining variables X¹ and X², R¹, R²), two R^{3a} are taken together to form a C₃-C₆ cycloalkyl group.

In some embodiments of Formula I (including the embodiments set forth above defining variables X¹ and X², R¹, R², and R^{3a}), variable R^{3b} is selected from C₁-C₃ alkyl optionally substituted with —OH or halogen. In some embodiments of Formula I (including the embodiments set forth above defining variables X¹ and X², R¹, R², and R^{3a}), R^{3b} is CH₃. In some embodiments of Formula I (including the embodiments set forth above defining variables X¹ and X², R¹, R², and R^{3a}), R^{3b} is selected from CF₂ and CF₃.

In some embodiments of Formula I, (including the embodiments set forth above defining variables X¹ and X², R¹, R², k, m, R^{3a}, and R³), three of variables R^{4a}, R⁴, R^{5a}, and R^{5b} are hydrogen and the remaining variable is chosen from C₁-C₄ alkyl groups. In some embodiments of Formula I, three of variables R^{4a}, R⁴, R^{5a}, and R^{5b} are hydrogen and the remaining variable is chosen from methyl, ethyl, cyclopropyl, fused cyclopropyl, and fused cyclobutyl. In some embodiments of Formula I, variables R^{4a}, R^{4b}, and R^{5b} are hydrogen and variable R^{5a} is chosen from methyl, ethyl, cyclopropyl, fused cyclopropyl, and fused cyclobutyl. In some embodiments of Formula I, variables R^{4b}, R^{5a}, and R^{5b} are hydrogen and variable R^{4a} is chosen from methyl, ethyl, cyclopropyl, fused cyclopropyl, and fused cyclobutyl. In some embodiments of Formula I, three of variables R^{4a}, R^{4b}, R^{5a}, and R^{5b} are hydrogen and the remaining variable is CH₃.

In the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im,

R⁶ is chosen from C₁-C₆ alkyl, —C(═O)O(C₁-C₄ alkyl), and

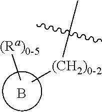

groups, wherein:
the C₁-C₆ alkyl of R⁶ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl (optionally substituted with 1 to 3 groups independently chosen from halogen and C₁-C₄ haloalkyl groups), —O—(C₆ aryl) (optionally substituted with 1 to 3 groups independently chosen from halogen and C₁-C₄ haloalkyl groups), 5- to 10-membered heterocyclyl, and 5- to 10-membered heteroaryl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
R^{a}, for each occurrence, is independently chosen from halogen, cyano, C₁-C₈ alkyl, C₁-C₆ haloalkyl, C₂-C₈ alkenyl, C₁-C₆ haloalkenyl, C₁-C₆ alkoxy, C₁-C₆ haloalkoxy, C₃-C₁₂ carbocyclyl, C₆ and C₁₀ aryl, 3- to 12-membered heterocyclyl, 5- to 10-membered heteroaryl, —C(═O)NR^{h}Rⁱ,
   —C(═O)OR^{k}, —C(═O)(C₁-C₄ alkylene)OR^{k}, —C(═O)R^{k}, —C(═O)(C₁-C₄ alkylene)S(═O)ₚR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —C(═O)C(═O)R^{k},—NR^{h}Rⁱ, —NH(CH₂)_{q}CHR^{h}Rⁱ, —NH(CH₂)_{q}NR^{h}Rⁱ, —NR^{h}C(═O)R^{k}, —NR^{h}C(═O)OR^{k}, —NR^{h}C(═O)(C₁-C₄ alkylene)OR^{k}, —NR^{h}C(═O)O(C₁-C₄ alkylene)R^{k}, —NR^{h}C(═O)NRⁱR^{j}, —NR^{h}C(═O)(C₁-C₄ alkylene)NRⁱS(═O)ₚR^{k}, —NR^{h}S(═O)ₚR^{k}, —NR^{h}C(═O)(C₁-C₄ alkylene)S(═O)ₚR^{k}, —NR^{h}S(═O)ₚ(C₁-C₄ alkylene)C(═O)OR^{k},
   —NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups), —NR^{h}C(═O)(C₁-C₆ alkylene)[O(CH₂)_{q}]ᵣOC(═O)—NR^{h}Rⁱ(CH₂)_{q}[O(CH₂)_{q}]ᵣ(C₁-C₆ alkyl) (optionally substituted by 1 to 3 R^{m} groups), —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, and —S(═O)ₚNR^{h}Rⁱ groups, wherein:
the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)-NRⁱS(═O)ₚR^{k}, —C(═O)(C₁-C₄ alkylene)-NR^{h}C(═O)R^{k}, —NR^{h}C(═O)O(C₁-C₄ alkylene)R^{k}, —NR^{h}C(═O)(C₁-C₄ alkylene)-OR^{k}, NR^{h}S(═O)ₚ(C₁-C₄ alkylene)C(═O)OR^{k}, and —NR^{h}C(═O)(C₁-C₄ alkylene)NRⁱS(═O)ₚR^{k} is optionally substituted with 1 to 3 —OH groups,
the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), —C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups; and
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, and 5- to 10-membered heterocyclyl groups, wherein:
R^{h}, Rⁱ, and R^{j}, for each occurrence, are each independently chosen from hydrogen, C₁-C₆ alkyl (optionally substituted with 1 to 4 R^{m} groups), C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 4 groups independently chosen from halogen, cyano, —OH, C₁-C₄ alkoxy, —C(═O)NH(C₁-C₄ alkyl), 5- to 10-membered heteroaryl (optionally substituted with 1 to 3 R^{m} groups), and 5- to 10-membered heterocyclyl (optionally substituted with 1 to 3 R^{m} groups) groups;
R^{k}, for each occurrence, is independently chosen from hydrogen, NH₂, (optionally substituted with 1 or 2 groups chosen from C₁-C₃ alkyl), C₁-C₆ alkyl, benzyl, C₆ aryl, C₃-C₆ carbocyclyl, 5- to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₆ alkoxy, C₃-C₆ cycloalkyl (optionally substituted with 1 to 3 halogen groups), 4- to 10-membered heterocyclyl (optionally substituted with 1 to 3 —OH groups), 5- to 10-membered aryl (optionally substituted with 1 to 3 groups chosen from C₁-C₄ alkyl and halogen), and 5- to-10-membered heteroaryl (optionally substituted with 1 to 3 —OH groups) groups; and
the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₄ alkyl (optionally substituted by 1 to 3 —OH groups), C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups,
the 5- to 10-membered heteroaryl and 5- to 10-membered heterocyclyl of any one of R^{k} are each optionally substituted with 1 to 3 groups independently chosen from halogen, oxo, cyano, —C(═O)CH₃, —NH₂, —OH, C₁-C₄ alkyl (optionally substituted by 1 to 3 —OH groups), C₁-C₄ haloalkyl, 5- to 10-membered heterocyclyl, and C₁-C₄ alkoxy groups;
R^{m}, for each occurrence, is independently chosen from halogen, cyano, oxo, —NH₂, C₁-C₆ alkyl, C₁-C₆ alkoxy, —C(═O)R^{k}, —S(═O)ₚR^{k}, —OR^{k}, and 5- to 10-membered heterocyclyl groups, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
p, for each occurrence, is an integer independently chosen from 1 and 2; and
q and r, for each occurrence, is an integer independently chosen from 0, 1, 2, and 3.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, —R⁶ is chosen from C₁-C₆ alkyl optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl (which is optionally substituted with 1 to 3 groups independently chosen from halogen and C₁-C₄ haloalkyl groups), —O—(C₆ aryl) (which is optionally substituted with 1 to 3 groups independently chosen from halogen and C₁-C₄ haloalkyl groups), 5- to 10-membered heterocyclyl, and 5- to 10-membered heteroaryl groups.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is an optionally substituted C₁-C₆ alkyl chosen from

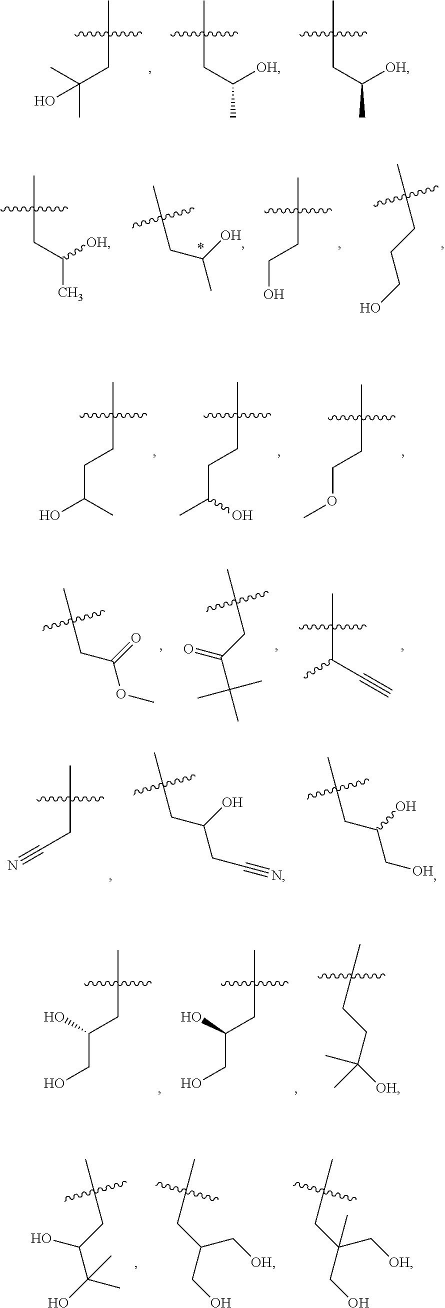

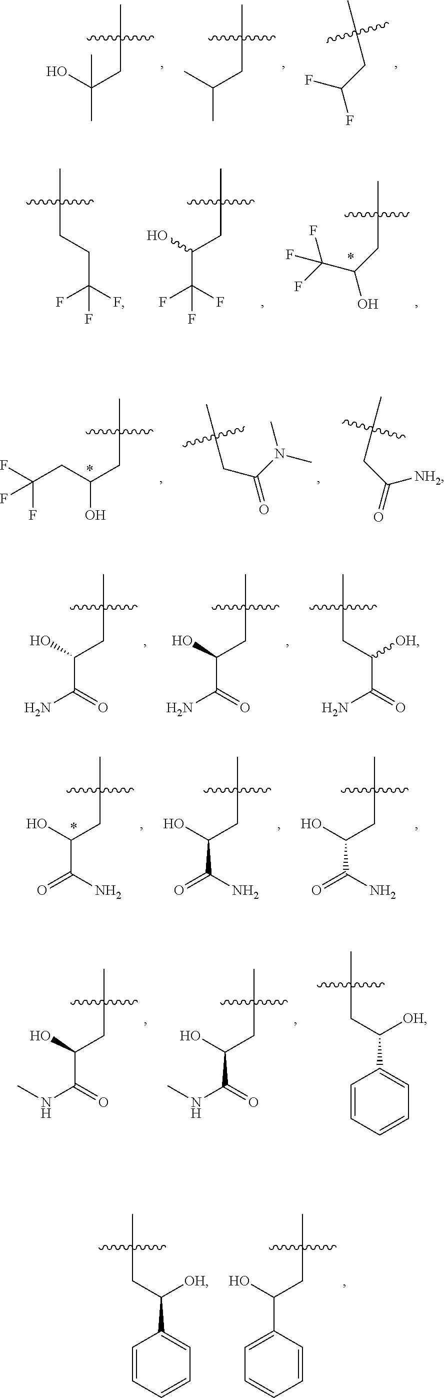

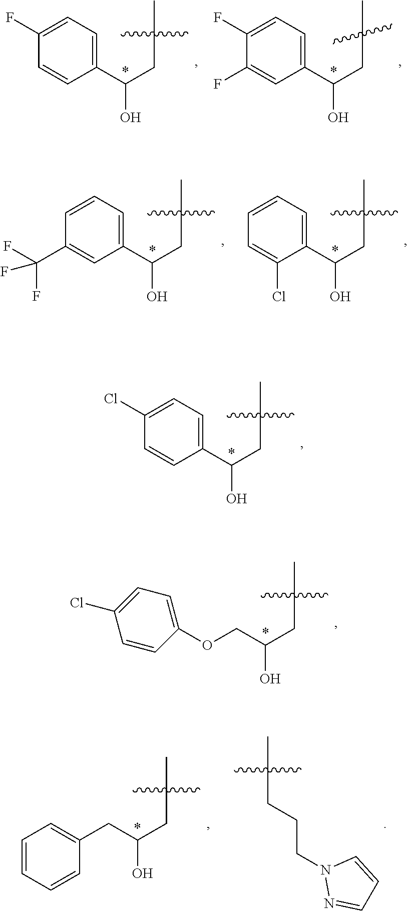

In some embodiments of Formula I (including the embodiments set forth above defining variables X¹ and X², R¹, R², k, m, R^{3a}, R^{3b}, R^{4a}, R^{4b}, R^{5a} and R^{5b}) and embodiments of Formulae Ia, Ib, Ic, Id, Ie, If, Ig, Ih, Ii, Ij, Ik, Il, and Im, R⁶ is chosen from —C(═O)O(C₁-C₄ alkyl).

In some embodiments of Formula I (including the embodiments set forth above defining variables X¹ and X², R¹, R², k, m, R^{3a}, R^{3b}, R^{4a}, R^{4b}, R^{5a} and R^{5b}) and embodiments of Formulae Ia, Ib, Ic, Id, Ie, If, Ig, Ih, Ii, Ij, Ik, Il, and Im, R⁶ is chosen from

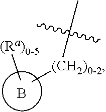

as defined for Formula I above. In some of these embodiments, Ring B is chosen from C₃-C₁₂ carbocyclyl optionally substituted with 1, 2, 3, 4, or 5 R^{a} groups as defined for Formula I. In some of these embodiments, Ring B is chosen from 3- to 12-membered heterocyclyl. In some of these embodiments, Ring B is chosen from C₆ and C₁₀ aryl optionally substituted with 1, 2, 3, 4, or 5 R^{a} groups as defined for Formula I. In some of these embodiments, Ring B is chosen from 5- to 10-membered heteroaryl groups optionally substituted with 1, 2, 3, 4, or 5 R^{a} groups as defined for Formula I.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is chosen from

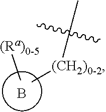

and Ring B is chosen from

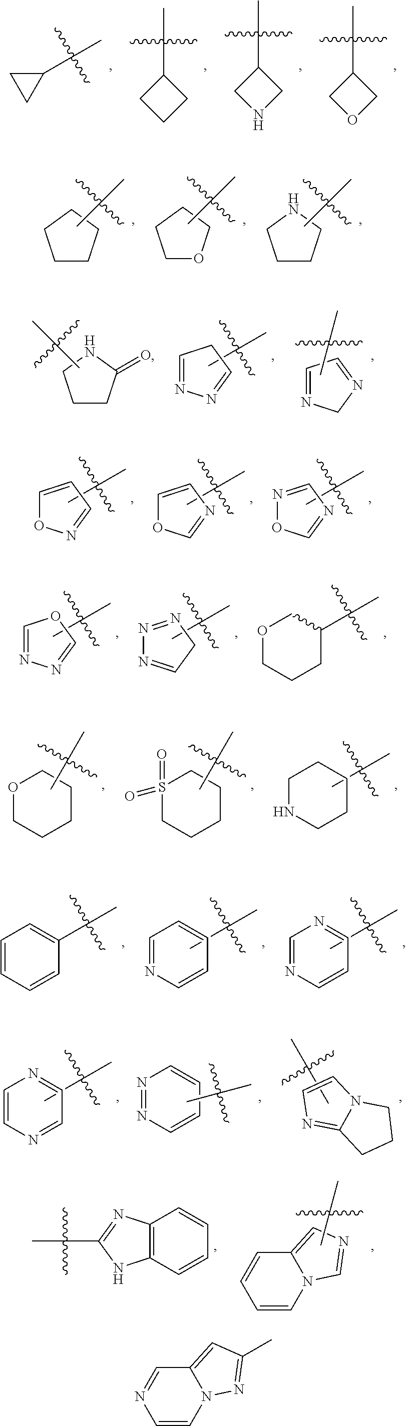

groups optionally substituted with 1, 2, 3, 4, or 5 R^{a} groups as defined for Formula I.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is chosen from

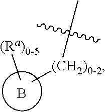

and Ring B is chosen from:

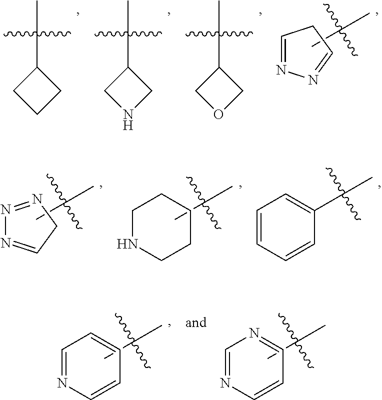

groups optionally substituted with 1, 2, 3, 4, or 5 R^{a} groups as defined for Formula I.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is chosen from:

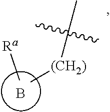

wherein Ring B is a 5-membered heteroaryl, and R^{a} is as defined for Formula I.

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is chosen from

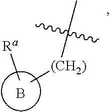

wherein Ring B is a 5-membered heteroaryl, and R^{a} is oxo or is chosen from C₁-C₅ alkyl, C₃-C₁₂ carbocyclyl and C₆ and C₁₀ aryl, each of which may be optionally substituted with 1 to 3 groups chosen from halogen and C₁-C₈ alkyl (wherein the C₁-C₈ alkyl may be optionally substituted with 1 to 3 groups chosen from halogen, —OH, SO₂CH₃, and SO₂NH₂).

In some embodiments of Formula I (including the embodiments set forth above defining variables X¹ and X², R¹, R², k, m, R^{3a}, R^{3b}, R^{4a}, R^{4b}, R^{5a}, and R^{5b}) and embodiments of Formulae Ia, Ib, Ic, Id, Ie, If, Ig, Ih, Ii, Ij, Ik, Il, and Im, R⁶ is chosen from:

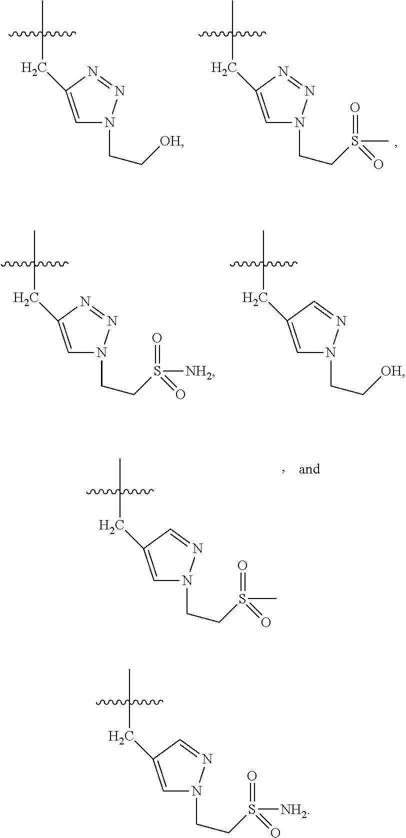 [SMILES: *Cc1cn(CCO)nn1 ; *Cc1cn(CCS(C)(=O)=O)nn1 ; *Cc1cn(CCS(N)(=O)=O)nn1 ; *Cc1cnn(CCO)c1 ; *Cc1cnn(CCS(C)(=O)=O)c1 ; *Cc1cnn(CCS(N)(=O)=O)c1]

In some embodiments of Formula I (including the embodiments set forth above defining variables X¹ and X², R¹, R², k, m, R^{3a}, R^{3b}, R^{4a}, R^{4b}, R^{5a}, R^{5b}, and R⁶) and embodiments of Formulae Ia, Ib, Ic, Id, Ie, If, Ig, Ih, Ii, Ij, Ik, Il, and Im, R^{a} is chosen from C₁-C₄ alkyl, halogen, —OH, and C₁-C₄ alkoxy, wherein the C₁-C₄ alkyl of R^{a} is optionally substituted with 1 to 3 polar groups, e.g. sulfones, sulfonamides, and alcohols.

In some embodiments of Formula I (including the embodiments set forth above defining variables X¹ and X², R¹, R², k, m, R^{3a}, R^{3b}, R^{4a}, R^{4b}, R^{5a}, R^{5b}, and R⁶) and embodiments of Formulae Ia, Ib, Ic, Id, Ie, If, Ig, Ih, Ii, Ij, Ik, Il, and Im, R^{a} is C₁-C₆ alkyl, optionally substituted as defined for Formula T. In some embodiments, the C₁-C₆ alkyl of R^{a} is optionally substituted with 1 to 3 groups selected from —OH, —SO₂CH₃, C₁-C₃ alkoxy, C(═O)NHCH₃, —SO₂NHCH₂CH₂OH, —SCF₃, —S CH₂C(CH₂)₂OH, —SO₂phenyl, 4-6 membered heterocycles (optionally substituted with 1 to 3 R^{m} groups), 4-6 membered heteroaryls (optionally substituted with 1 to 3 R^{m} groups), cyano, NHC(═O)-4-6 membered heteroaryl.

In some embodiments of Formula I (including the embodiments set forth above defining variables X¹ and X², R¹, R², k, m, R^{3a}, R^{3b}, R^{4a}, R^{4b}, R^{5a}, R^{5b}, and R⁶) and embodiments of Formulae Ia, Ib, Ic, Id, Ie, If, Ig, Ih, Ii, Ij, Ik, Il, and Im, R^{a} is chosen from 4-6 membered carobcycles, 4-6 membered heterocycles and 4-6 membered heteroaryls, any of which is optionally substituted as defined for Formula I.

In some embodiments, the at least one compound, tautomer, deuterated derivative, or pharmaceutically acceptable salt of the disclosure is chosen from Compounds 1 to 1183 depicted in Table 1, a tautomer thereof, a deuterated derivative of that compound or tautomer, or a pharmaceutically acceptable salt of any of the foregoing. A wavy line in a compound in Table 1

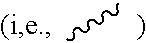

depicts a bond between two atoms and indicates a position of mixed stereochemistry for a collection of molecules, such as a racemic mixture, cis/trans isomers, or (E)/(Z) isomers. An asterisk adjacent to an atom

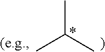

in a compound in Table 1, indicates a chiral position in the molecule.

In some embodiments, the compound of Formula I is selected from the compounds presented in Table I below, tautomers of those compounds, deuterated derivatives of those compounds and tautomers, and pharmaceutically acceptable salts of any of the foregoing.

TABLE 1
Compounds 1 to 1183

| | |
|---|---|
| 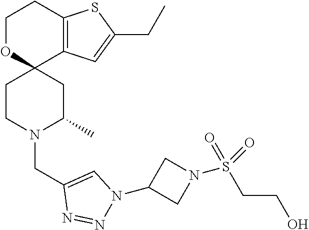 [SMILES: CCc1cc2c(s1)CCO[C@@]21CCN(Cc2cn(C3CN(S(=O)(=O)CCO)C3)nn2)[C@@H](C)C1] | 1 |
| 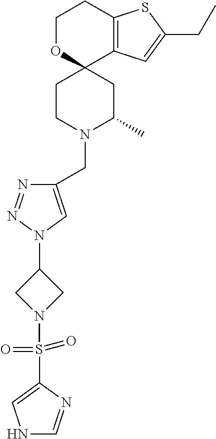 [SMILES: CCc1cc2c(s1)CCO[C@@]21CCN(Cc2cn(C3CN(S(=O)(=O)c4c[nH]cn4)C3)nn2)[C@@H](C)C1] | 2 |
| 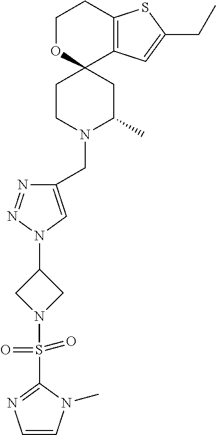 [SMILES: CCc1cc2c(s1)CCO[C@@]21CCN(Cc2cn(C3CN(S(=O)(=O)c4nccn4C)C3)nn2)[C@@H](C)C1] | 3 |
| 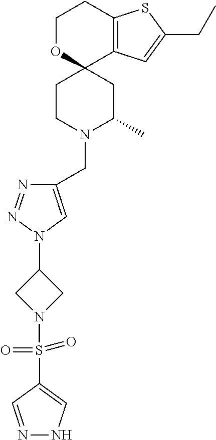 [SMILES: CCc1cc2c(s1)CCO[C@@]21CCN(Cc2cn(C3CN(S(=O)(=O)c4cn[nH]c4)C3)nn2)[C@@H](C)C1] | 4 |
| 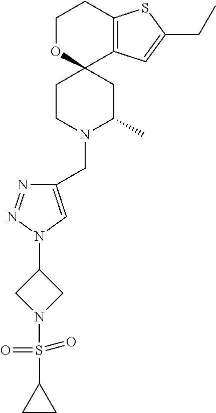 [SMILES: CCc1cc2c(s1)CCO[C@@]21CCN(Cc2cn(C3CN(S(=O)(=O)C4CC4)C3)nn2)[C@@H](C)C1] | 5 |
| 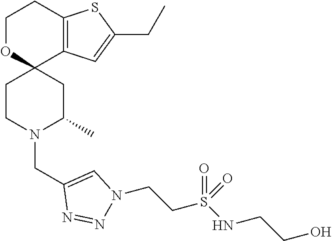 [SMILES: CCc1cc2c(s1)CCO[C@@]21CCN(Cc2cn(CCS(=O)(=O)NCCO)nn2)[C@@H](C)C1] | 6 |
| 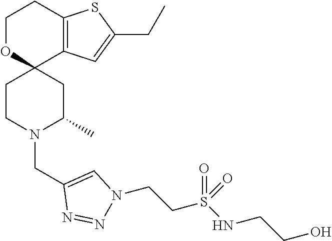 [SMILES: CCc1cc2c(s1)CCO[C@@]21CCN(Cc2cn(CCS(=O)(=O)NCCO)nn2)[C@@H](C)C1] | 7 |
| 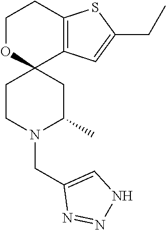 [SMILES: CCc1cc2c(s1)CCO[C@@]21CCN(Cc2c[nH]nn2)[C@@H](C)C1] | 8 |
| 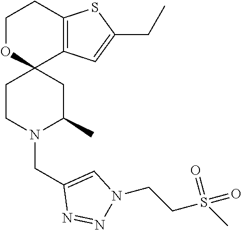 [SMILES: CCc1cc2c(s1)CCO[C@@]21CCN(Cc2cn(CCS(C)(=O)=O)nn2)[C@H](C)C1] | 9 |
| 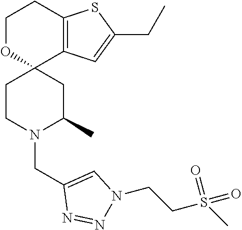 [SMILES: CCc1cc2c(s1)CCO[C@]21CCN(Cc2cn(CCS(C)(=O)=O)nn2)[C@H](C)C1] | 10 |
| 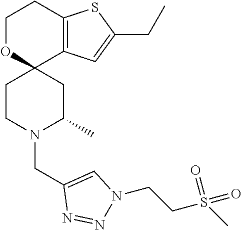 [SMILES: CCc1cc2c(s1)CCO[C@@]21CCN(Cc2cn(CCS(C)(=O)=O)nn2)[C@@H](C)C1] | 11 |
| 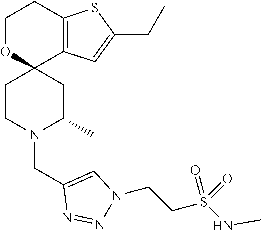 [SMILES: CCc1cc2c(s1)CCO[C@@]21CCN(Cc2cn(CCS(=O)(=O)NC)nn2)[C@@H](C)C1] | 12 |
| 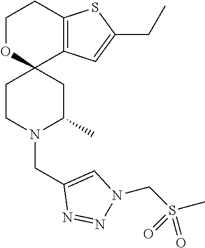 [SMILES: CCc1cc2c(s1)CCO[C@@]21CCN(Cc2cn(CS(C)(=O)=O)nn2)[C@@H](C)C1] | 13 |
| 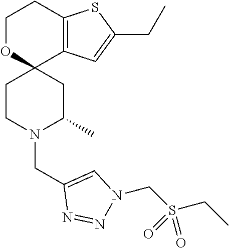 [SMILES: CCc1cc2c(s1)CCO[C@@]21CCN(Cc2cn(CS(=O)(=O)CC)nn2)[C@@H](C)C1] | 14 |
| 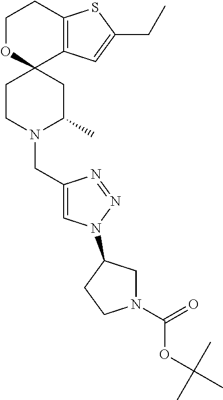 [SMILES: CCc1cc2c(s1)CCO[C@@]21CCN(Cc2cn([C@@H]3CCN(C(=O)OC(C)(C)C)C3)nn2)[C@@H](C)C1] | 15 |
| 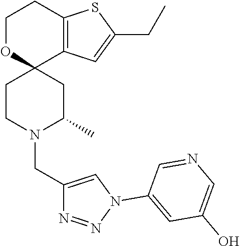 [SMILES: CCc1cc2c(s1)CCO[C@@]21CCN(Cc2cn(-c3cncc(O)c3)nn2)[C@@H](C)C1] | 16 |
| 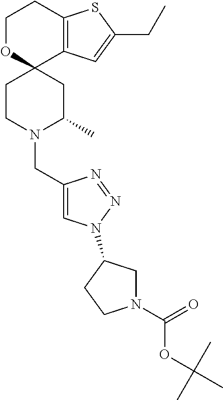 [SMILES: CCc1cc2c(s1)CCO[C@@]21CCN(Cc2cn([C@H]3CCN(C(=O)OC(C)(C)C)C3)nn2)[C@@H](C)C1] | 17 |
| 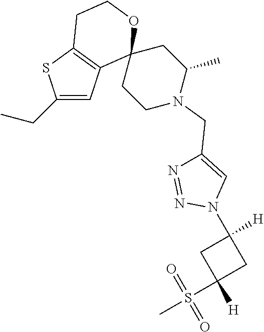 [SMILES: CCc1cc2c(s1)CCO[C@@]21CCN(Cc2cn([C@H]3C[C@H](S(C)(=O)=O)C3)nn2)[C@@H](C)C1] | 18 |
| 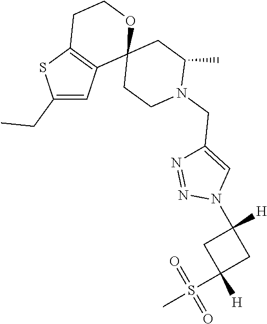 [SMILES: CCc1cc2c(s1)CCO[C@@]21CCN(Cc2cn([C@H]3C[C@@H](S(C)(=O)=O)C3)nn2)[C@@H](C)C1] | 19 |
| 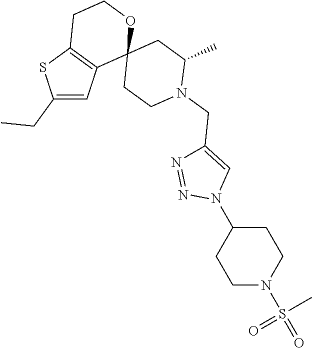 [SMILES: CCc1cc2c(s1)CCO[C@@]21CCN(Cc2cn(C3CCN(S(C)(=O)=O)CC3)nn2)[C@@H](C)C1] | 20 |
| 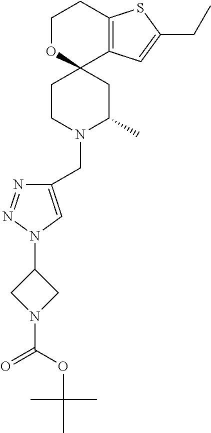 [SMILES: CCc1cc2c(s1)CCO[C@@]21CCN(Cc2cn(C3CN(C(=O)OC(C)(C)C)C3)nn2)[C@@H](C)C1] | 21 |
| 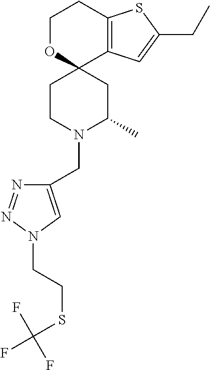 [SMILES: CCc1cc2c(s1)CCO[C@@]21CCN(Cc2cn(CCSC(F)(F)F)nn2)[C@@H](C)C1] | 22 |
| 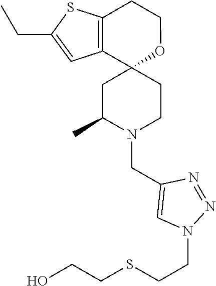 [SMILES: CCc1cc2c(s1)CCO[C@@]21CCN(Cc2cn(CCSCCO)nn2)[C@@H](C)C1] | 23 |
| 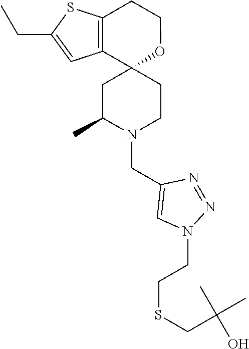 [SMILES: CCc1cc2c(s1)CCO[C@@]21CCN(Cc2cn(CCSCC(C)(C)O)nn2)[C@@H](C)C1] | 24 |
| 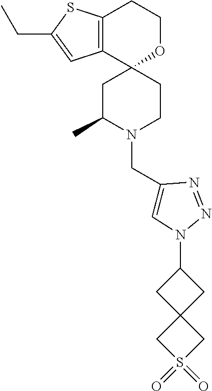 [SMILES: CCc1cc2c(s1)CCO[C@@]21CCN(Cc2cn(C3CC4(C3)CS(=O)(=O)C4)nn2)[C@@H](C)C1] | 25 |
| 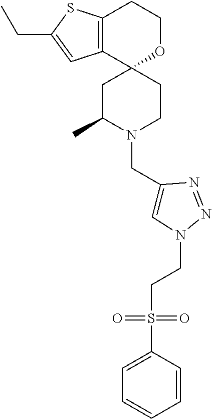 [SMILES: CCc1cc2c(s1)CCO[C@@]21CCN(Cc2cn(CCS(=O)(=O)c3ccccc3)nn2)[C@@H](C)C1] | 26 |
| 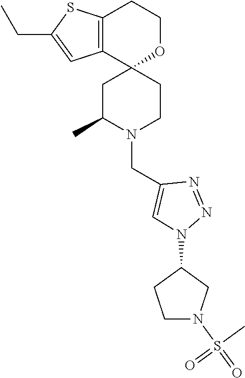 [SMILES: CCc1cc2c(s1)CCO[C@@]21CCN(Cc2cn([C@H]3CCN(S(C)(=O)=O)C3)nn2)[C@@H](C)C1] | 27 |
| 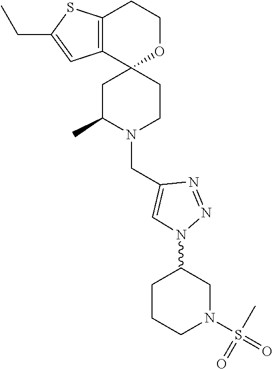 [SMILES: CCc1cc2c(s1)CCO[C@@]21CCN(Cc2cn(C3CCCN(S(C)(=O)=O)C3)nn2)[C@@H](C)C1] | 28 |
| 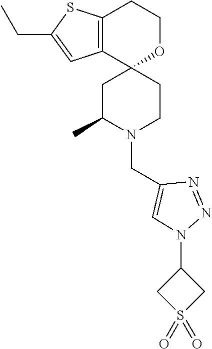 [SMILES: CCc1cc2c(s1)CCO[C@@]21CCN(Cc2cn(C3CS(=O)(=O)C3)nn2)[C@@H](C)C1] | 29 |
| 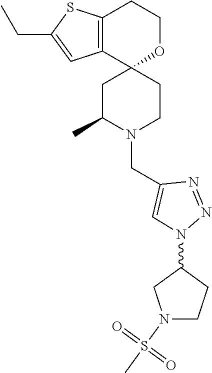 [SMILES: CCc1cc2c(s1)CCO[C@@]21CCN(Cc2cn(C3CCN(S(C)(=O)=O)C3)nn2)[C@@H](C)C1] | 30 |
| 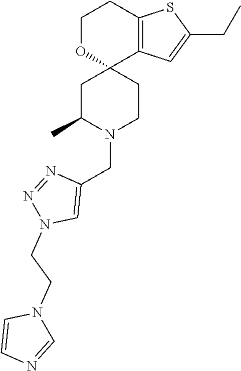 [SMILES: CCc1cc2c(s1)CCO[C@@]21CCN(Cc2cn(CCn3ccnc3)nn2)[C@@H](C)C1] | 31 |
| 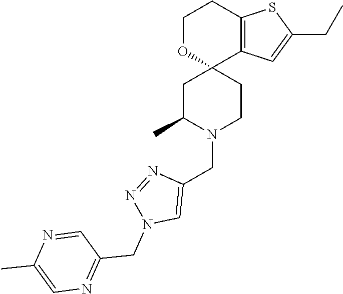 [SMILES: CCc1cc2c(s1)CCO[C@@]21CCN(Cc2cn(Cc3cnc(C)cn3)nn2)[C@@H](C)C1] | 32 |
| 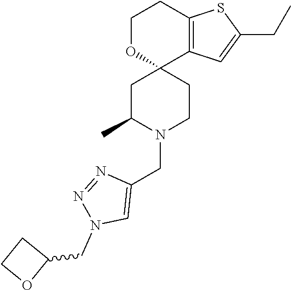 [SMILES: CCc1cc2c(s1)CCO[C@@]21CCN(Cc2cn(CC3CCO3)nn2)[C@@H](C)C1] | 33 |
| 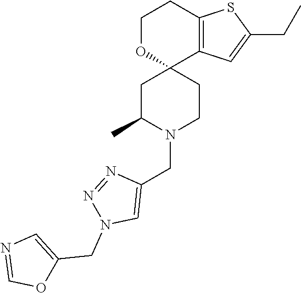 [SMILES: CCc1cc2c(s1)CCO[C@@]21CCN(Cc2cn(Cc3cnco3)nn2)[C@@H](C)C1] | 34 |
| 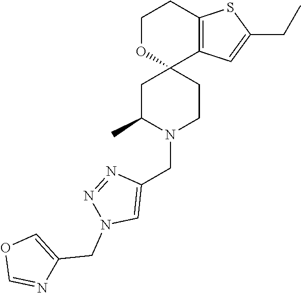 [SMILES: CCc1cc2c(s1)CCO[C@@]21CCN(Cc2cn(Cc3cocn3)nn2)[C@@H](C)C1] | 35 |
| 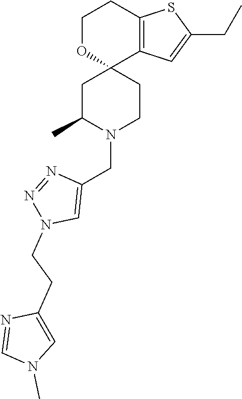 [SMILES: CCc1cc2c(s1)CCO[C@@]21CCN(Cc2cn(CCc3cn(C)cn3)nn2)[C@@H](C)C1] | 36 |
| 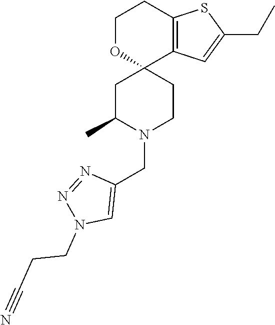 [SMILES: CCc1cc2c(s1)CCO[C@@]21CCN(Cc2cn(CCC#N)nn2)[C@@H](C)C1] | 37 |
| 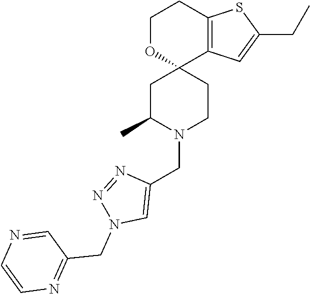 [SMILES: CCc1cc2c(s1)CCO[C@@]21CCN(Cc2cn(Cc3cnccn3)nn2)[C@@H](C)C1] | 38 |
| 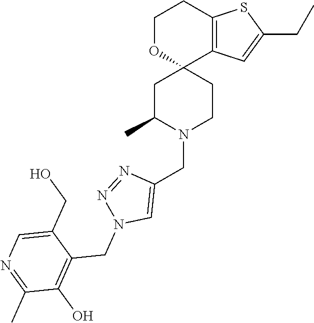 [SMILES: CCc1cc2c(s1)CCO[C@@]21CCN(Cc2cn(Cc3c(CO)cnc(C)c3O)nn2)[C@@H](C)C1] | 39 |
| 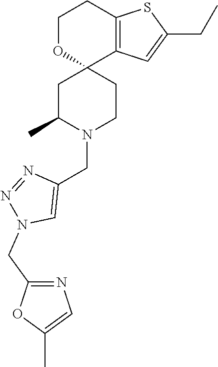 [SMILES: CCc1cc2c(s1)CCO[C@@]21CCN(Cc2cn(Cc3ncc(C)o3)nn2)[C@@H](C)C1] | 40 |
| 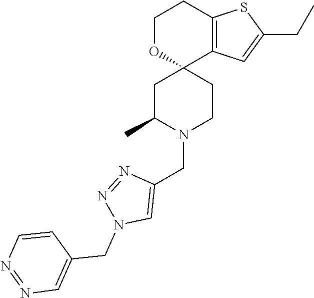 [SMILES: CCc1cc2c(s1)CCO[C@@]21CCN(Cc2cn(Cc3ccnnc3)nn2)[C@@H](C)C1] | 41 |
| 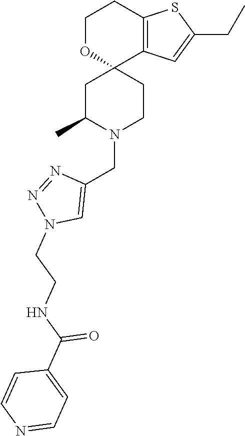 [SMILES: CCc1cc2c(s1)CCO[C@@]21CCN(Cc2cn(CCNC(=O)c3ccncc3)nn2)[C@@H](C)C1] | 42 |
| 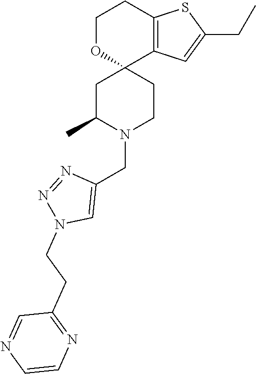 [SMILES: CCc1cc2c(s1)CCO[C@@]21CCN(Cc2cn(CCc3cnccn3)nn2)[C@@H](C)C1] | 43 |
| 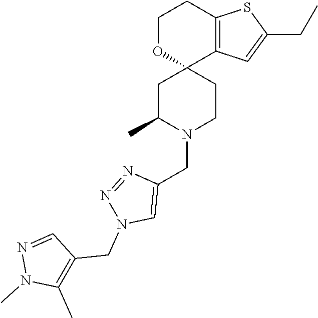 [SMILES: CCc1cc2c(s1)CCO[C@@]21CCN(Cc2cn(Cc3cnn(C)c3C)nn2)[C@@H](C)C1] | 44 |
| 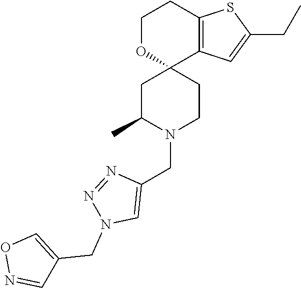 [SMILES: CCc1cc2c(s1)CCO[C@@]21CCN(Cc2cn(Cc3cnoc3)nn2)[C@@H](C)C1] | 45 |
| 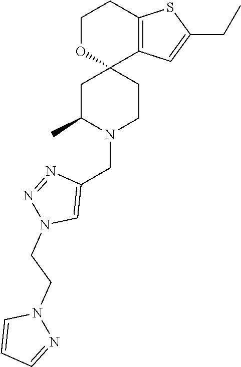 [SMILES: CCc1cc2c(s1)CCO[C@@]21CCN(Cc2cn(CCn3cccn3)nn2)[C@@H](C)C1] | 46 |
| 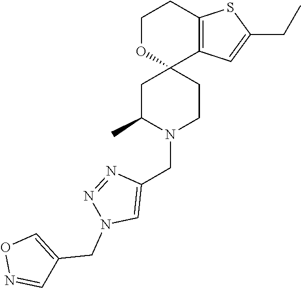 [SMILES: CCc1cc2c(s1)CCO[C@@]21CCN(Cc2cn(Cc3cnoc3)nn2)[C@@H](C)C1] | 47 |
| 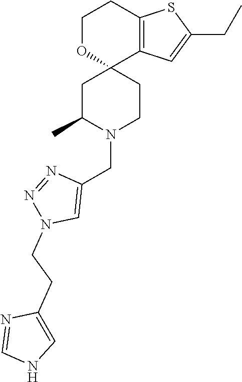 [SMILES: CCc1cc2c(s1)CCO[C@@]21CCN(Cc2cn(CCc3c[nH]cn3)nn2)[C@@H](C)C1] | 48 |
| 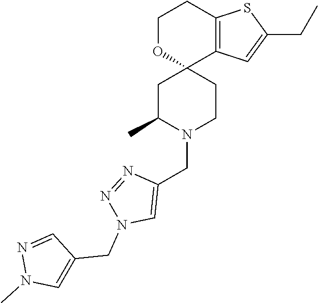 [SMILES: CCc1cc2c(s1)CCO[C@@]21CCN(Cc2cn(Cc3cnn(C)c3)nn2)[C@@H](C)C1] | 49 |
| 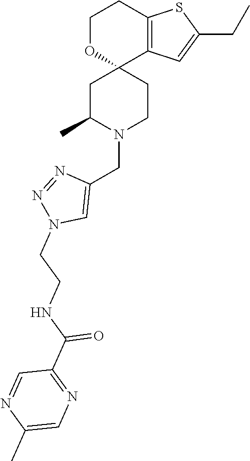 [SMILES: CCc1cc2c(s1)CCO[C@@]21CCN(Cc2cn(CCNC(=O)c3cnc(C)cn3)nn2)[C@@H](C)C1] | 50 |
| 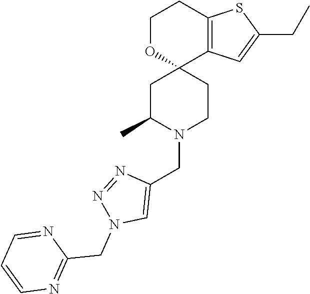 [SMILES: CCc1cc2c(s1)CCO[C@@]21CCN(Cc2cn(Cc3ncccn3)nn2)[C@@H](C)C1] | 51 |
| 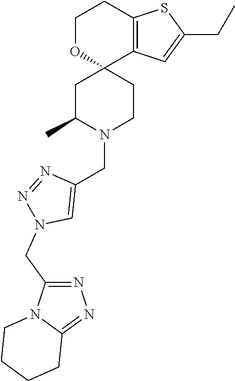 [SMILES: CCc1cc2c(s1)CCO[C@@]21CCN(Cc2cn(Cc3nnc4n3CCCC4)nn2)[C@@H](C)C1] | 52 |
| 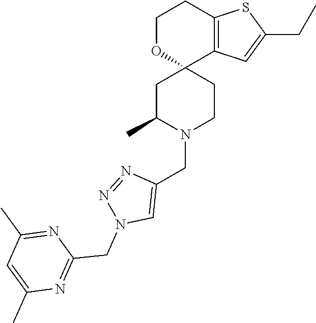 [SMILES: CCc1cc2c(s1)CCO[C@@]21CCN(Cc2cn(Cc3nc(C)cc(C)n3)nn2)[C@@H](C)C1] | 53 |
| 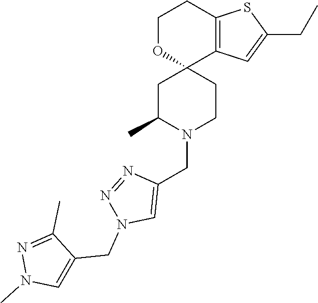 [SMILES: CCc1cc2c(s1)CCO[C@@]21CCN(Cc2cn(Cc3cn(C)nc3C)nn2)[C@@H](C)C1] | 54 |
| 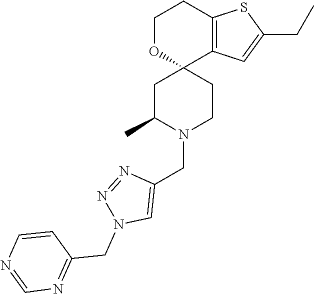 [SMILES: CCc1cc2c(s1)CCO[C@@]21CCN(Cc2cn(Cc3ccncn3)nn2)[C@@H](C)C1] | 55 |
| 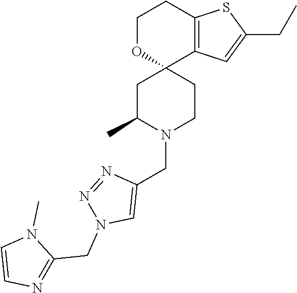 [SMILES: CCc1cc2c(s1)CCO[C@@]21CCN(Cc2cn(Cc3nccn3C)nn2)[C@@H](C)C1] | 56 |
| 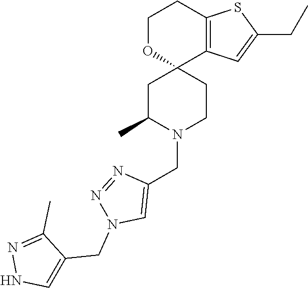 [SMILES: CCc1cc2c(s1)CCO[C@@]21CCN(Cc2cn(Cc3c[nH]nc3C)nn2)[C@@H](C)C1] | 57 |
| 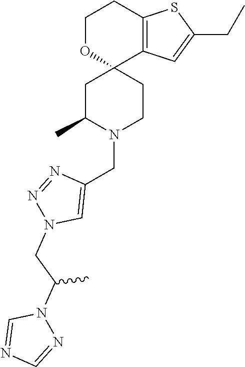 [SMILES: CCc1cc2c(s1)CCO[C@@]21CCN(Cc2cn(CC(C)n3cncn3)nn2)[C@@H](C)C1] | 58 |
| 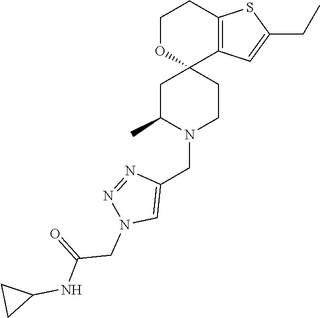 [SMILES: CCc1cc2c(s1)CCO[C@@]21CCN(Cc2cn(CC(=O)NC3CC3)nn2)[C@@H](C)C1] | 59 |
| 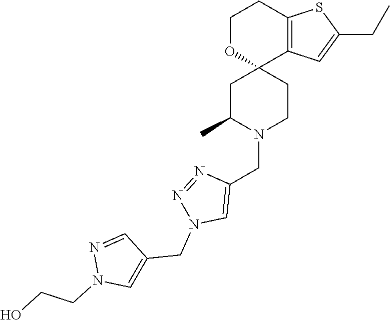 [SMILES: CCc1cc2c(s1)CCO[C@@]21CCN(Cc2cn(Cc3cnn(CCO)c3)nn2)[C@@H](C)C1] | 60 |
| 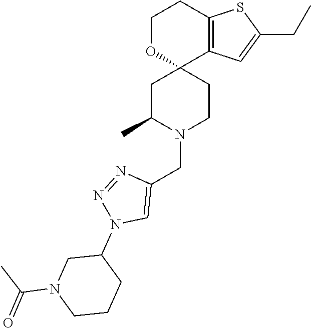 [SMILES: CCc1cc2c(s1)CCO[C@@]21CCN(Cc2cn(C3CCCN(C(C)=O)C3)nn2)[C@@H](C)C1] | 61 |
| 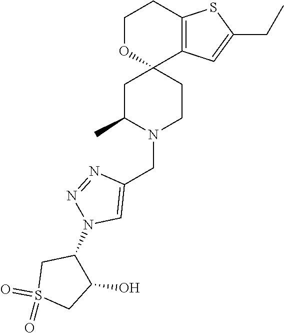 [SMILES: CCc1cc2c(s1)CCO[C@@]21CCN(Cc2cn([C@H]3CS(=O)(=O)C[C@H]3O)nn2)[C@@H](C)C1] | 62 |
| 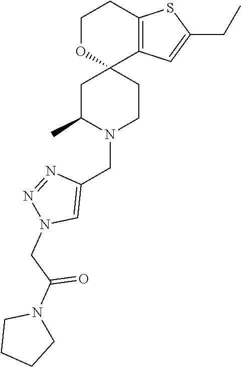 [SMILES: CCc1cc2c(s1)CCO[C@@]21CCN(Cc2cn(CC(=O)N3CCCC3)nn2)[C@@H](C)C1] | 63 |
| 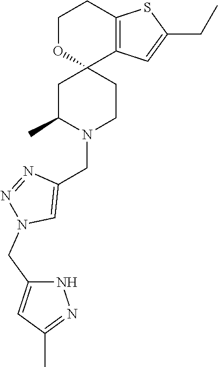 [SMILES: CCc1cc2c(s1)CCO[C@@]21CCN(Cc2cn(Cc3cc(C)n[nH]3)nn2)[C@@H](C)C1] | 64 |
| 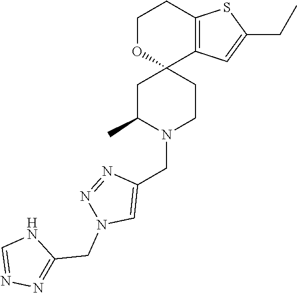 [SMILES: CCc1cc2c(s1)CCO[C@@]21CCN(Cc2cn(Cc3nnc[nH]3)nn2)[C@@H](C)C1] | 65 |
| 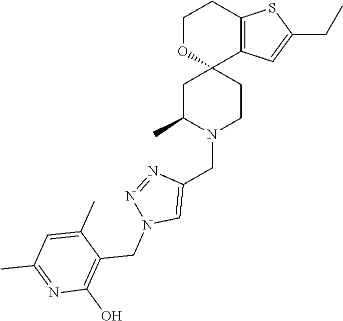 [SMILES: CCc1cc2c(s1)CCO[C@@]21CCN(Cc2cn(Cc3c(C)cc(C)nc3O)nn2)[C@@H](C)C1] | 66 |
| 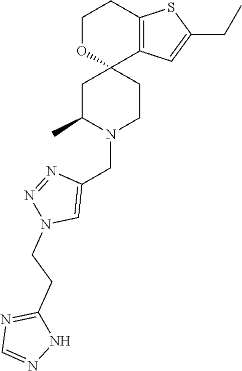 [SMILES: CCc1cc2c(s1)CCO[C@@]21CCN(Cc2cn(CCc3ncn[nH]3)nn2)[C@@H](C)C1] | 67 |
| 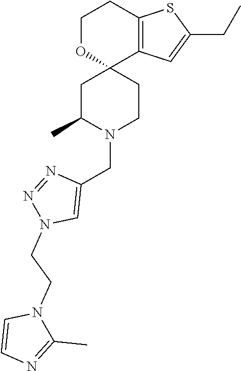 [SMILES: CCc1cc2c(s1)CCO[C@@]21CCN(Cc2cn(CCn3ccnc3C)nn2)[C@@H](C)C1] | 68 |
| 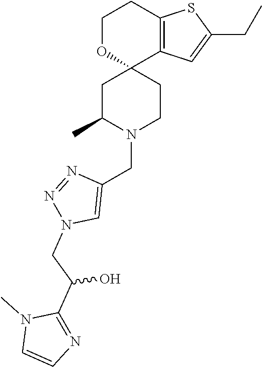 [SMILES: CCc1cc2c(s1)CCO[C@@]21CCN(Cc2cn(CC(O)c3nccn3C)nn2)[C@@H](C)C1] | 69 |
| 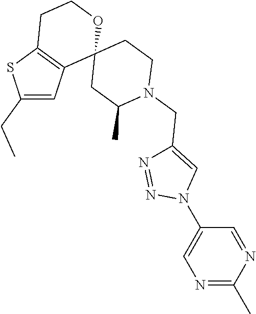 [SMILES: CCc1cc2c(s1)CCO[C@@]21CCN(Cc2cn(-c3cnc(C)nc3)nn2)[C@@H](C)C1] | 70 |
| 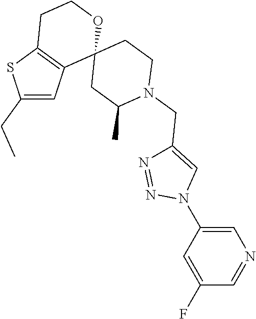 [SMILES: CCc1cc2c(s1)CCO[C@@]21CCN(Cc2cn(-c3cncc(F)c3)nn2)[C@@H](C)C1] | 71 |
| 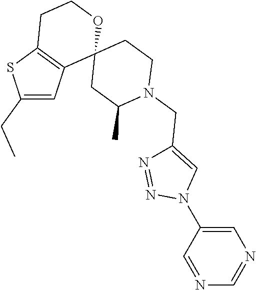 [SMILES: CCc1cc2c(s1)CCO[C@@]21CCN(Cc2cn(-c3cncnc3)nn2)[C@@H](C)C1] | 72 |
| 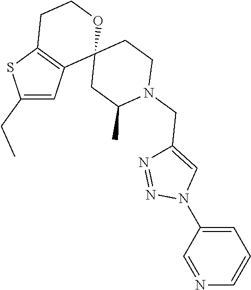 [SMILES: CCc1cc2c(s1)CCO[C@@]21CCN(Cc2cn(-c3cccnc3)nn2)[C@@H](C)C1] | 73 |
| 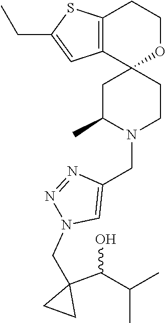 [SMILES: CCc1cc2c(s1)CCO[C@@]21CCN(Cc2cn(CC3(C(O)C(C)C)CC3)nn2)[C@@H](C)C1] | 74 |
| 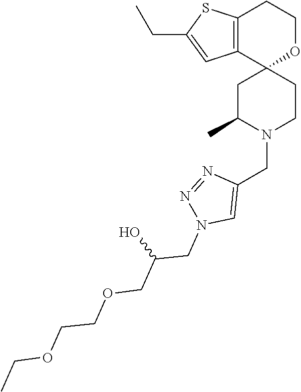 | 75 |
| 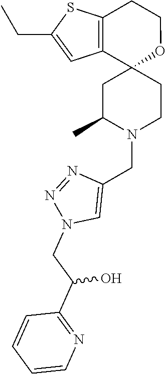 | 76 |
| 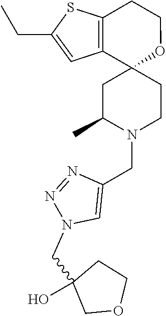 | 77 |
| 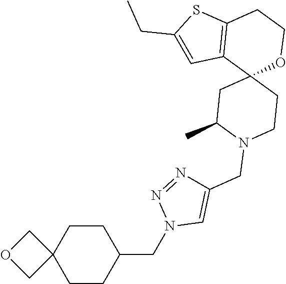 | 78 |
| 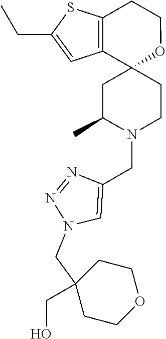 | 79 |
| 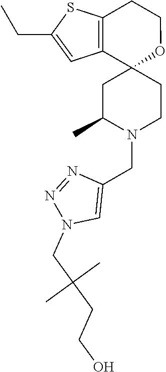 | 80 |
| 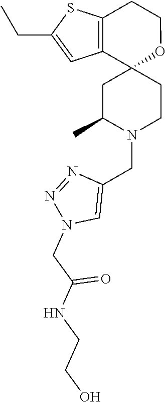 | 81 |
| 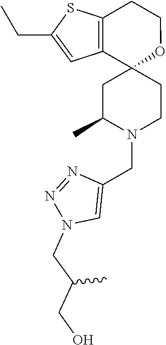 | 82 |
| 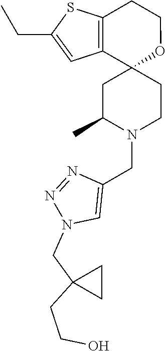 | 83 |
| 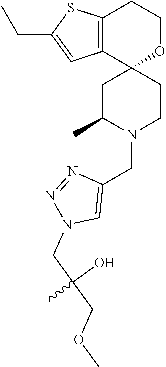 | 84 |
| 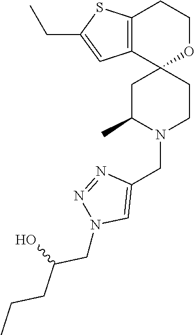 [SMILES: CCCC(O)Cn1cc(CN2CC[C@]3(C[C@@H]2C)OCCc2sc(CC)cc23)nn1] | 85 |
| 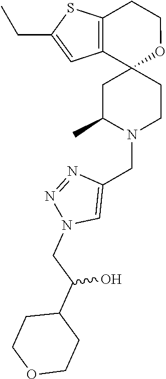 | 86 |
| 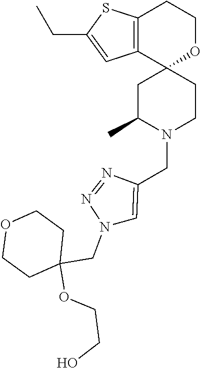 | 87 |
| 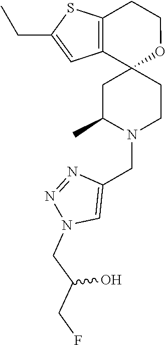 | 88 |
| 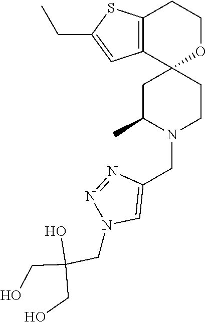 [SMILES: CCc1cc2c(s1)CCO[C@@]21CCN(Cc2cn(CC(O)(CO)CO)nn2)[C@@H](C)C1] | 89 |
| 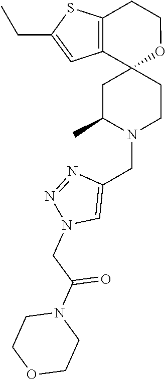 | 90 |
| 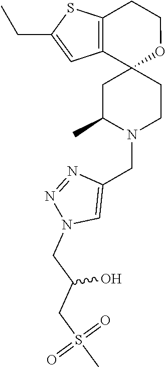 | 91 |
| 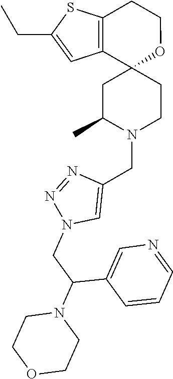 | 92 |
| 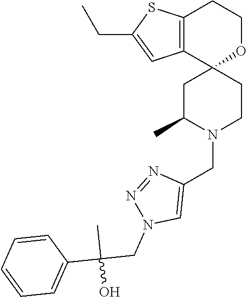 | 93 |
| 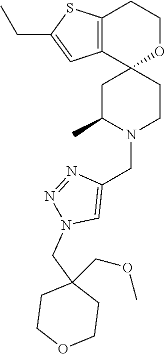 | 94 |
| 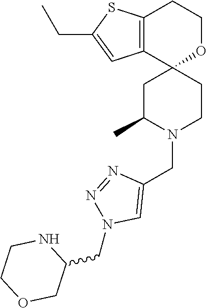 [SMILES: CCc1cc2c(s1)CCO[C@@]21CCN(Cc2cn(CC3COCCN3)nn2)[C@@H](C)C1] | 95 |
| 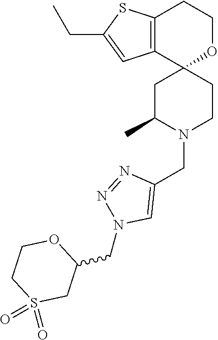 [SMILES: CCc1cc2c(s1)CCO[C@@]21CCN(Cc2cn(CC3CS(=O)(=O)CCO3)nn2)[C@@H](C)C1] | 96 |
| 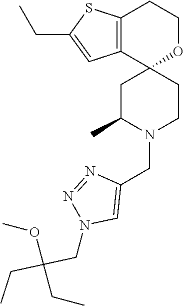 [SMILES: CCc1cc2c(s1)CCO[C@@]21CCN(Cc2cn(CC(CC)(CC)OC)nn2)[C@@H](C)C1] | 97 |
| 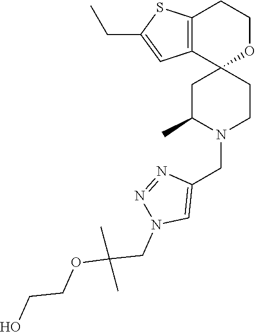 | 98 |
| 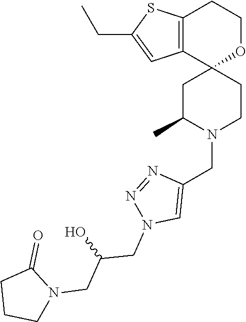 | 99 |
| 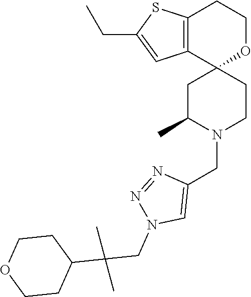 | 100 |
| 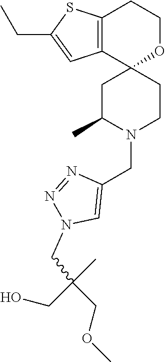 | 101 |
| 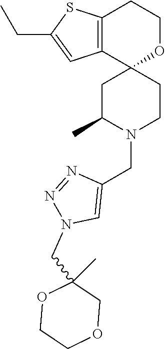 | 102 |
| 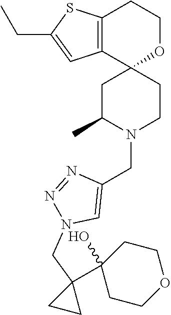 | 103 |
| 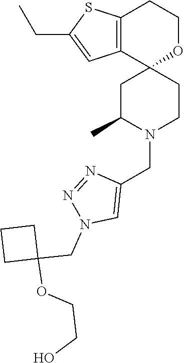 | 104 |
| 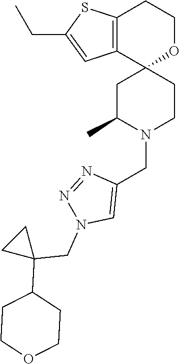 | 105 |
| 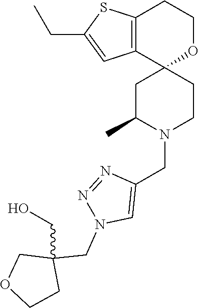 | 106 |
| 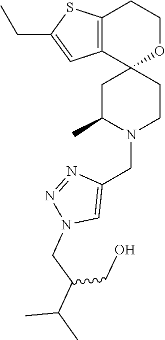 | 107 |
| 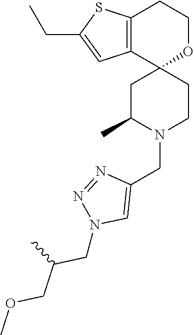 [SMILES: CCc1cc2c(s1)CCO[C@@]21CCN(Cc2cn(CC(C)COC)nn2)[C@@H](C)C1] | 108 |
| 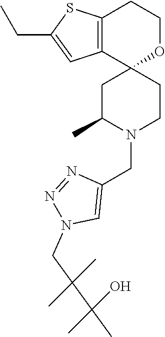 | 109 |
| 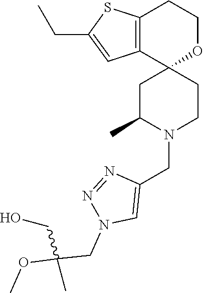 | 110 |
| 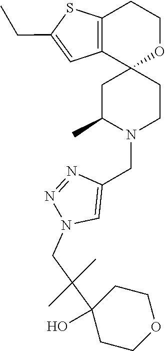 | 111 |
| 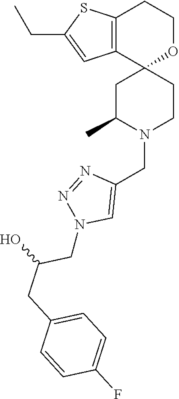 [SMILES: CCc1cc2c(s1)CCO[C@@]21CCN(Cc2cn(CC(O)Cc3ccc(F)cc3)nn2)[C@@H](C)C1] | 112 |
| 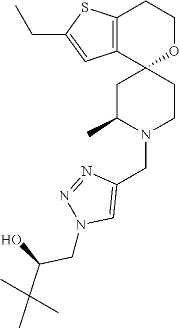 [SMILES: CCc1cc2c(s1)CCO[C@@]21CCN(Cc2cn(C[C@H](O)C(C)(C)C)nn2)[C@@H](C)C1] | 113 |
| 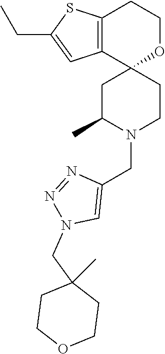 | 114 |
| 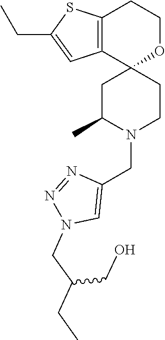 | 115 |
| 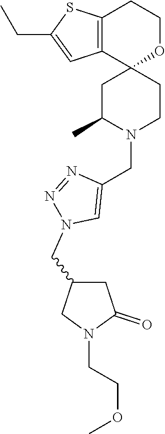 | 116 |
| 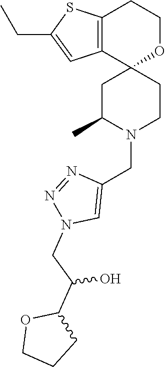 | 117 |
| 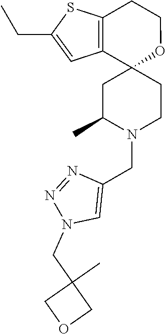 | 118 |
| 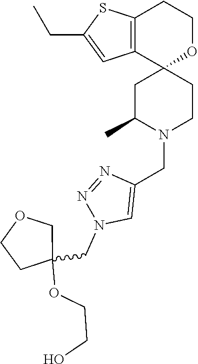 | 119 |
| 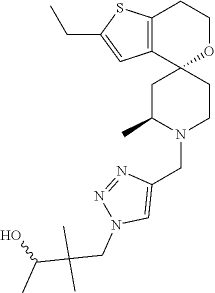 | 120 |
| 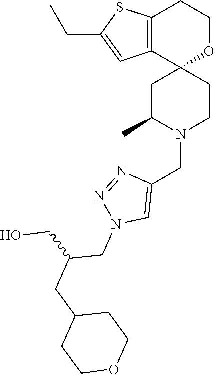 | 121 |
| 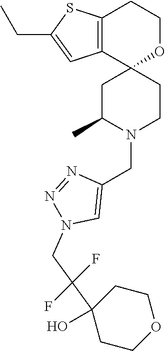 | 122 |
| 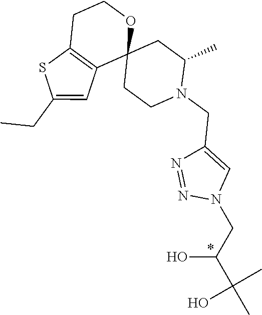 | 123 |
| 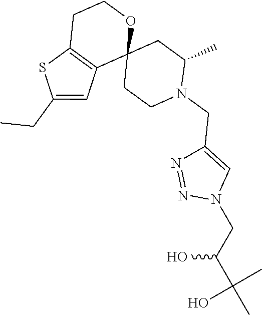 | 124 |
| 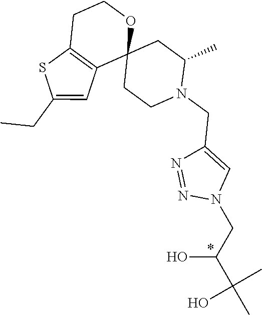 | 125 |
| 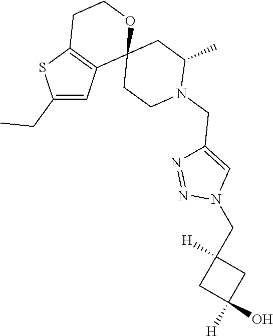 [SMILES: CCc1cc2c(s1)CCO[C@@]21CCN(Cc2cn(C[C@H]3C[C@@H](O)C3)nn2)[C@@H](C)C1] | 126 |
| 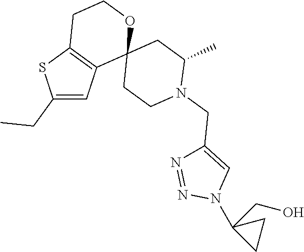 [SMILES: CCc1cc2c(s1)CCO[C@@]21CCN(Cc2cn(C3(CO)CC3)nn2)[C@@H](C)C1] | 127 |
| 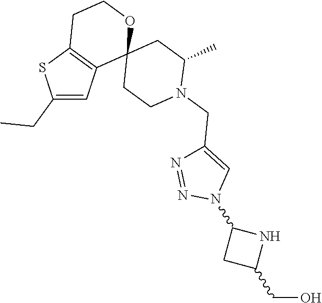 [SMILES: CCc1cc2c(s1)CCO[C@@]21CCN(Cc2cn(C3CC(CO)N3)nn2)[C@@H](C)C1] | 128 |
| 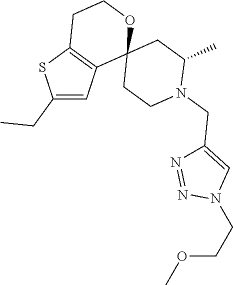 [SMILES: CCc1cc2c(s1)CCO[C@@]21CCN(Cc2cn(CCOC)nn2)[C@@H](C)C1] | 129 |
| 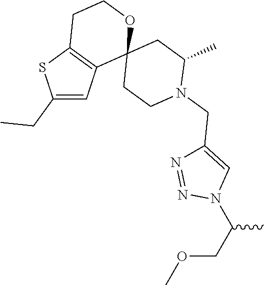 [SMILES: CCc1cc2c(s1)CCO[C@@]21CCN(Cc2cn(C(C)COC)nn2)[C@@H](C)C1] | 130 |
| 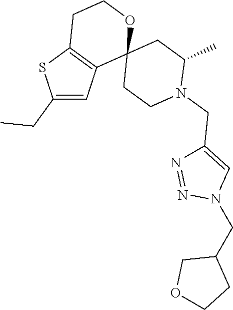 [SMILES: CCc1cc2c(s1)CCO[C@@]21CCN(Cc2cn(CC3CCOC3)nn2)[C@@H](C)C1] | 131 |
| 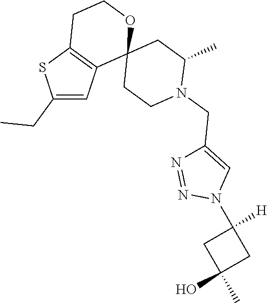 [SMILES: CCc1cc2c(s1)CCO[C@@]21CCN(Cc2cn([C@H]3C[C@@](C)(O)C3)nn2)[C@@H](C)C1] | 132 |
| 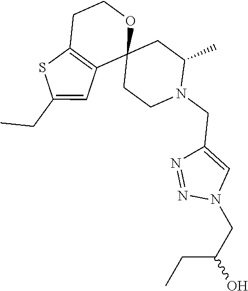 | 133 |
| 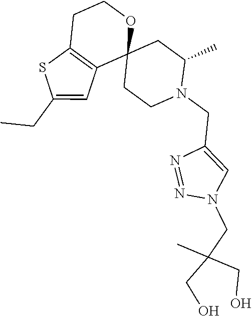 | 134 |
| 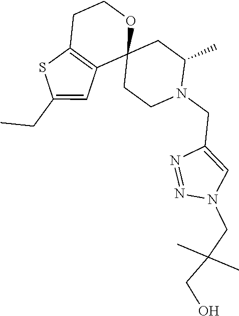 | 135 |
| 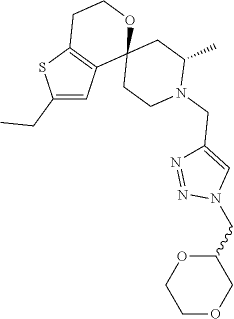 [SMILES: CCc1cc2c(s1)CCO[C@@]21CCN(Cc2cn(CC3COCCO3)nn2)[C@@H](C)C1] | 136 |
| 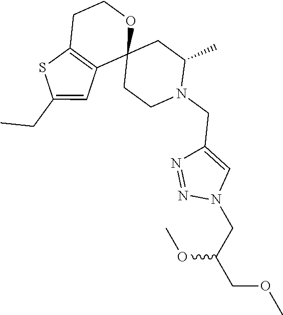 | 137 |
| 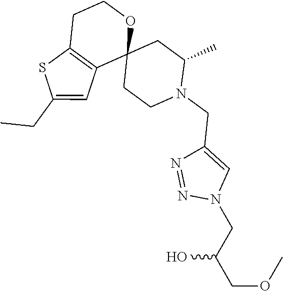 | 138 |
| 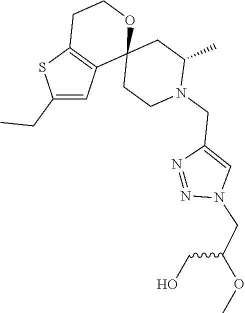 [SMILES: CCc1cc2c(s1)CCO[C@@]21CCN(Cc2cn(CC(CO)OC)nn2)[C@@H](C)C1] | 139 |
| 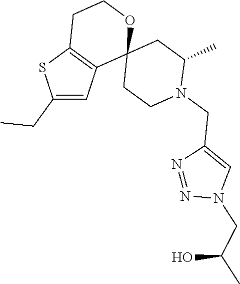 [SMILES: CCc1cc2c(s1)CCO[C@@]21CCN(Cc2cn(C[C@@H](C)O)nn2)[C@@H](C)C1] | 140 |
| 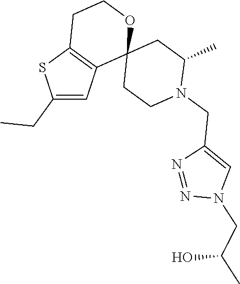 [SMILES: CCc1cc2c(s1)CCO[C@@]21CCN(Cc2cn(C[C@H](C)O)nn2)[C@@H](C)C1] | 141 |
| 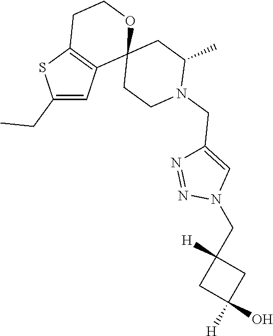 [SMILES: CCc1cc2c(s1)CCO[C@@]21CCN(Cc2cn(C[C@H]3C[C@H](O)C3)nn2)[C@@H](C)C1] | 142 |
| 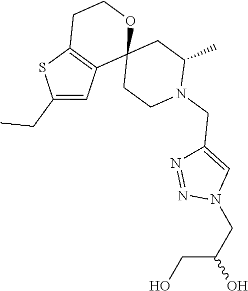 | 143 |
| 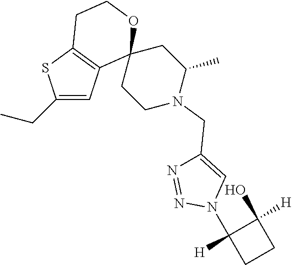 [SMILES: CCc1cc2c(s1)CCO[C@@]21CCN(Cc2cn([C@H]3CC[C@@H]3O)nn2)[C@@H](C)C1] | 144 |
| 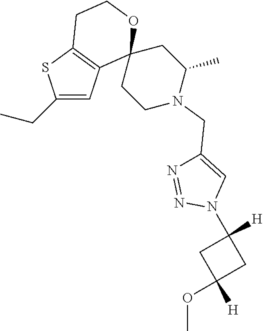 [SMILES: CCc1cc2c(s1)CCO[C@@]21CCN(Cc2cn([C@H]3C[C@@H](OC)C3)nn2)[C@@H](C)C1] | 145 |
| 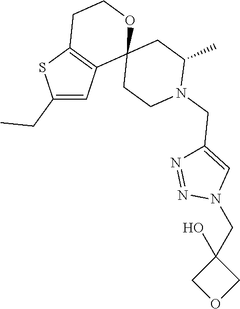 [SMILES: CCc1cc2c(s1)CCO[C@@]21CCN(Cc2cn(CC3(O)COC3)nn2)[C@@H](C)C1] | 146 |
| 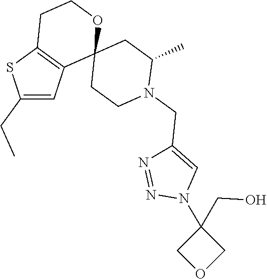 [SMILES: CCc1cc2c(s1)CCO[C@@]21CCN(Cc2cn(C3(CO)COC3)nn2)[C@@H](C)C1] | 147 |
| 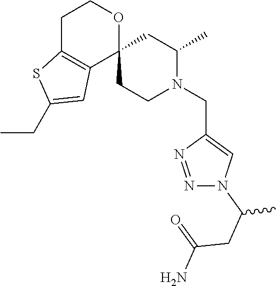 | 148 |
| 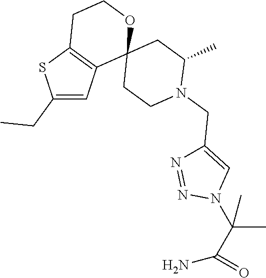 [SMILES: CCc1cc2c(s1)CCO[C@@]21CCN(Cc2cn(C(C)(C)C(N)=O)nn2)[C@@H](C)C1] | 149 |
| 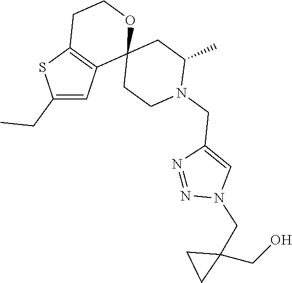 [SMILES: CCc1cc2c(s1)CCO[C@@]21CCN(Cc2cn(CC3(CO)CC3)nn2)[C@@H](C)C1] | 150 |
| 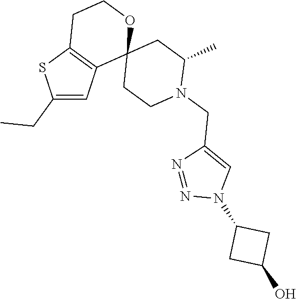 [SMILES: CCc1cc2c(s1)CCO[C@@]21CCN(Cc2cn([C@H]3C[C@H](O)C3)nn2)[C@@H](C)C1] | 151 |
| 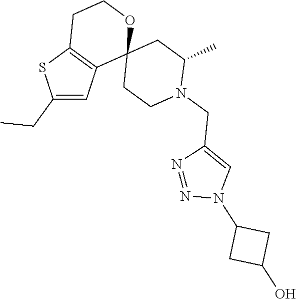 [SMILES: CCc1cc2c(s1)CCO[C@@]21CCN(Cc2cn(C3CC(O)C3)nn2)[C@@H](C)C1] | 152 |
| 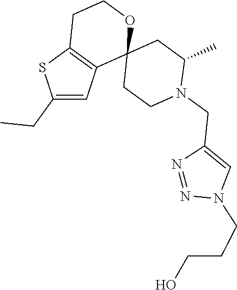 [SMILES: CCc1cc2c(s1)CCO[C@@]21CCN(Cc2cn(CCCO)nn2)[C@@H](C)C1] | 153 |
| 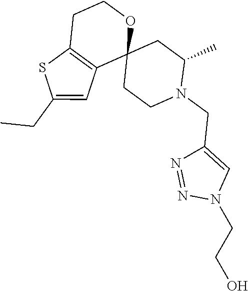 | 154 |
| 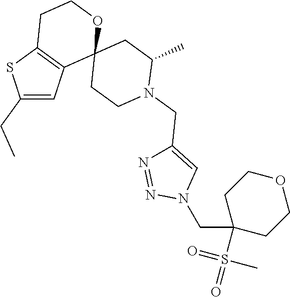 [SMILES: CCc1cc2c(s1)CCO[C@@]21CCN(Cc2cn(CC3(S(C)(=O)=O)CCOCC3)nn2)[C@@H](C)C1] | 155 |
| 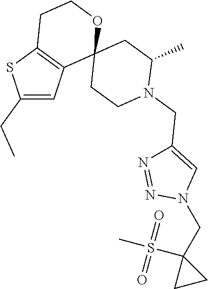 [SMILES: CCc1cc2c(s1)CCO[C@@]21CCN(Cc2cn(CC3(S(C)(=O)=O)CC3)nn2)[C@@H](C)C1] | 156 |
| 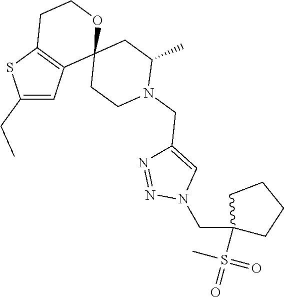 [SMILES: CCc1cc2c(s1)CCO[C@@]21CCN(Cc2cn(CC3(S(C)(=O)=O)CCCC3)nn2)[C@@H](C)C1] | 157 |
| 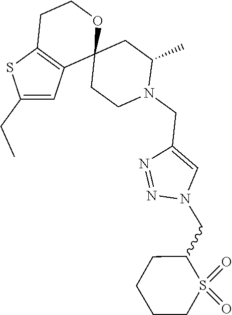 [SMILES: CCc1cc2c(s1)CCO[C@@]21CCN(Cc2cn(CC3CCCCS3(=O)=O)nn2)[C@@H](C)C1] | 158 |
| 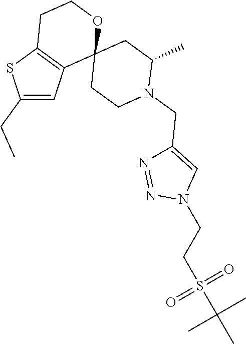 [SMILES: CCc1cc2c(s1)CCO[C@@]21CCN(Cc2cn(CCS(=O)(=O)C(C)(C)C)nn2)[C@@H](C)C1] | 159 |
| 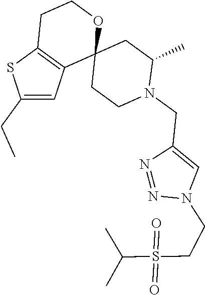 [SMILES: CCc1cc2c(s1)CCO[C@@]21CCN(Cc2cn(CCS(=O)(=O)C(C)C)nn2)[C@@H](C)C1] | 160 |
| 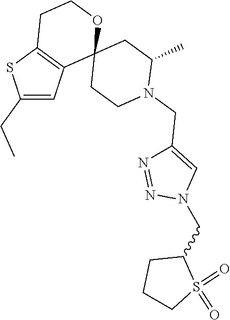 [SMILES: CCc1cc2c(s1)CCO[C@@]21CCN(Cc2cn(CC3CCCS3(=O)=O)nn2)[C@@H](C)C1] | 161 |
| 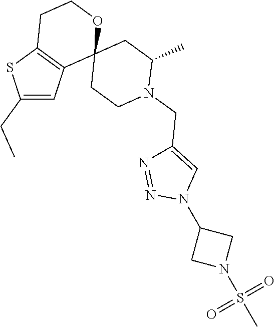 [SMILES: CCc1cc2c(s1)CCO[C@@]21CCN(Cc2cn(C3CN(S(C)(=O)=O)C3)nn2)[C@@H](C)C1] | 162 |
| 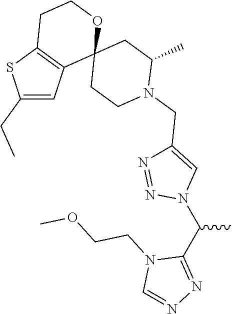 [SMILES: CCc1cc2c(s1)CCO[C@@]21CCN(Cc2cn(C(C)c3nncn3CCOC)nn2)[C@@H](C)C1] | 163 |
| 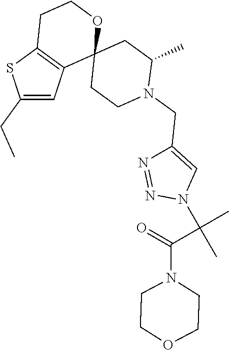 [SMILES: CCc1cc2c(s1)CCO[C@@]21CCN(Cc2cn(C(C)(C)C(=O)N3CCOCC3)nn2)[C@@H](C)C1] | 164 |
| 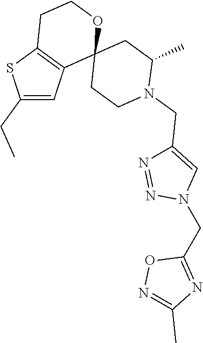 [SMILES: CCc1cc2c(s1)CCO[C@@]21CCN(Cc2cn(Cc3nc(C)no3)nn2)[C@@H](C)C1] | 165 |
| 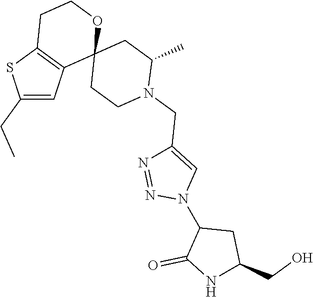 [SMILES: CCc1cc2c(s1)CCO[C@@]21CCN(Cc2cn(C3C[C@@H](CO)NC3=O)nn2)[C@@H](C)C1] | 166 |
| 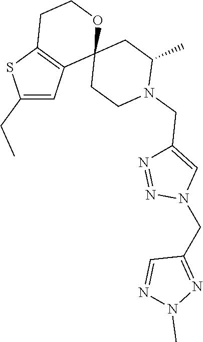 [SMILES: CCc1cc2c(s1)CCO[C@@]21CCN(Cc2cn(Cc3cnn(C)n3)nn2)[C@@H](C)C1] | 167 |
| 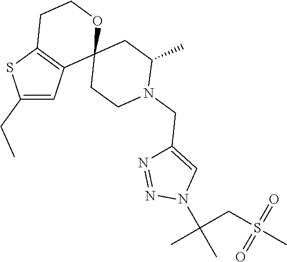 [SMILES: CCc1cc2c(s1)CCO[C@@]21CCN(Cc2cn(C(C)(C)CS(C)(=O)=O)nn2)[C@@H](C)C1] | 168 |
| 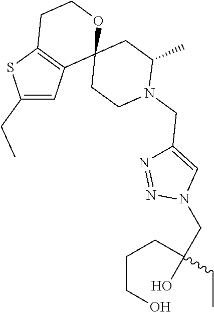 [SMILES: CCc1cc2c(s1)CCO[C@@]21CCN(Cc2cn(CC(O)(CC)CCCO)nn2)[C@@H](C)C1] | 169 |
| 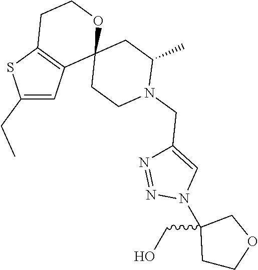 [SMILES: CCc1cc2c(s1)CCO[C@@]21CCN(Cc2cn(C3(CO)CCOC3)nn2)[C@@H](C)C1] | 170 |
| 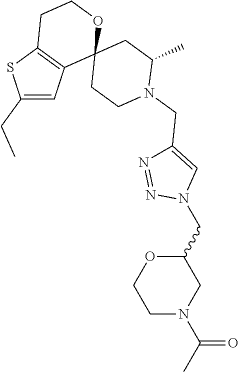 [SMILES: CCc1cc2c(s1)CCO[C@@]21CCN(Cc2cn(CC3CN(C(C)=O)CCO3)nn2)[C@@H](C)C1] | 171 |
| 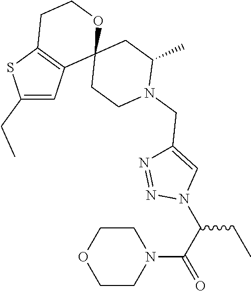 [SMILES: CCc1cc2c(s1)CCO[C@@]21CCN(Cc2cn(C(CC)C(=O)N3CCOCC3)nn2)[C@@H](C)C1] | 172 |
| 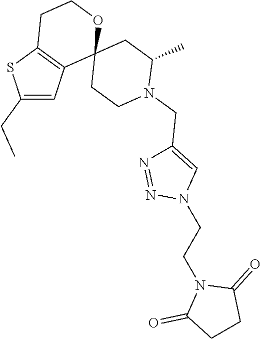 [SMILES: CCc1cc2c(s1)CCO[C@@]21CCN(Cc2cn(CCN3C(=O)CCC3=O)nn2)[C@@H](C)C1] | 173 |
| 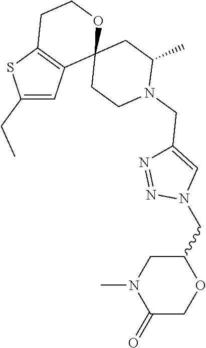 [SMILES: CCc1cc2c(s1)CCO[C@@]21CCN(Cc2cn(CC3CN(C)C(=O)CO3)nn2)[C@@H](C)C1] | 174 |
| 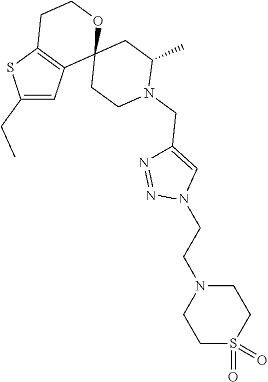 [SMILES: CCc1cc2c(s1)CCO[C@@]21CCN(Cc2cn(CCN3CCS(=O)(=O)CC3)nn2)[C@@H](C)C1] | 175 |
| 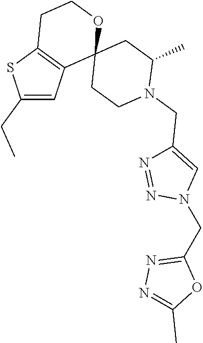 [SMILES: CCc1cc2c(s1)CCO[C@@]21CCN(Cc2cn(Cc3nnc(C)o3)nn2)[C@@H](C)C1] | 176 |
| 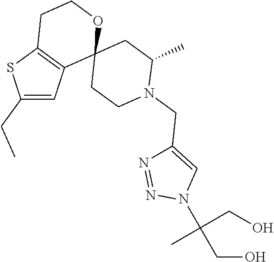 [SMILES: CCc1cc2c(s1)CCO[C@@]21CCN(Cc2cn(C(C)(CO)CO)nn2)[C@@H](C)C1] | 177 |
| 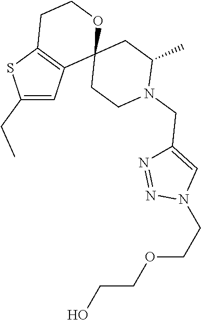 [SMILES: CCc1cc2c(s1)CCO[C@@]21CCN(Cc2cn(CCOCCO)nn2)[C@@H](C)C1] | 178 |
| 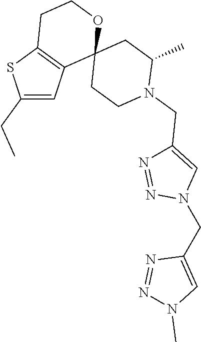 [SMILES: CCc1cc2c(s1)CCO[C@@]21CCN(Cc2cn(Cc3cn(C)nn3)nn2)[C@@H](C)C1] | 179 |
| 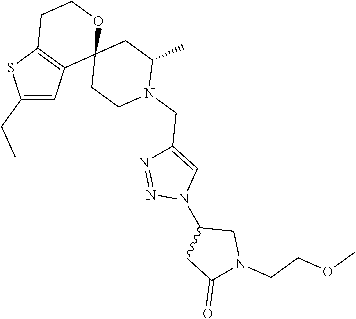 [SMILES: CCc1cc2c(s1)CCO[C@@]21CCN(Cc2cn(C3CC(=O)N(CCOC)C3)nn2)[C@@H](C)C1] | 180 |
| 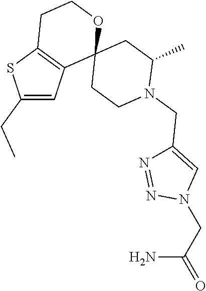 [SMILES: CCc1cc2c(s1)CCO[C@@]21CCN(Cc2cn(CC(N)=O)nn2)[C@@H](C)C1] | 181 |
| 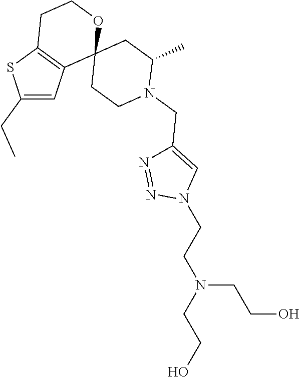 [SMILES: CCc1cc2c(s1)CCO[C@@]21CCN(Cc2cn(CCN(CCO)CCO)nn2)[C@@H](C)C1] | 182 |
| 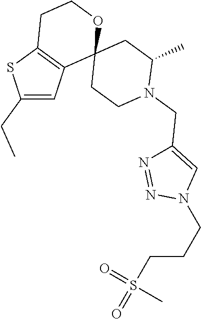 [SMILES: CCc1cc2c(s1)CCO[C@@]21CCN(Cc2cn(CCCS(C)(=O)=O)nn2)[C@@H](C)C1] | 183 |
| 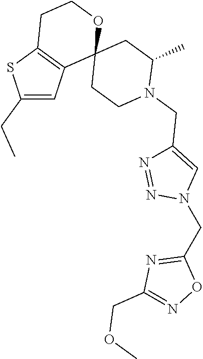 [SMILES: CCc1cc2c(s1)CCO[C@@]21CCN(Cc2cn(Cc3nc(COC)no3)nn2)[C@@H](C)C1] | 184 |
| 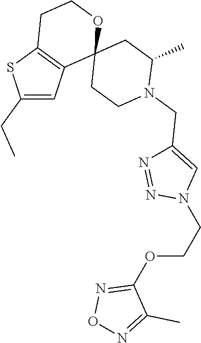 [SMILES: CCc1cc2c(s1)CCO[C@@]21CCN(Cc2cn(CCOc3nonc3C)nn2)[C@@H](C)C1] | 185 |
| 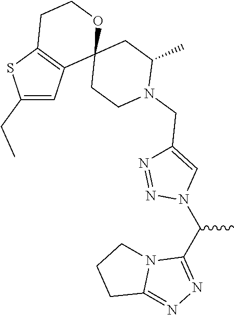 [SMILES: CCc1cc2c(s1)CCO[C@@]21CCN(Cc2cn(C(C)c3nnc4n3CCC4)nn2)[C@@H](C)C1] | 186 |
| 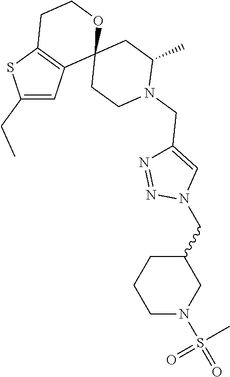 [SMILES: CCc1cc2c(s1)CCO[C@@]21CCN(Cc2cn(CC3CCCN(S(C)(=O)=O)C3)nn2)[C@@H](C)C1] | 187 |
| 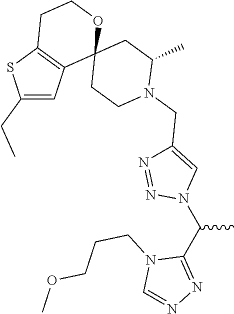 [SMILES: CCc1cc2c(s1)CCO[C@@]21CCN(Cc2cn(C(C)c3nncn3CCCOC)nn2)[C@@H](C)C1] | 188 |
| 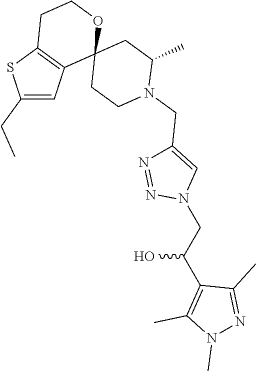 [SMILES: CCc1cc2c(s1)CCO[C@@]21CCN(Cc2cn(CC(O)c3c(C)nn(C)c3C)nn2)[C@@H](C)C1] | 189 |
| 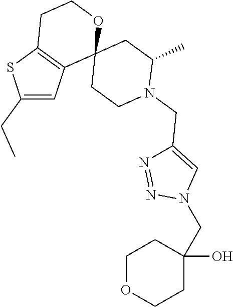 [SMILES: CCc1cc2c(s1)CCO[C@@]21CCN(Cc2cn(CC3(O)CCOCC3)nn2)[C@@H](C)C1] | 190 |
| 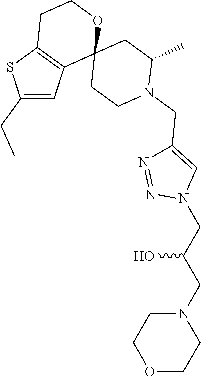 [SMILES: CCc1cc2c(s1)CCO[C@@]21CCN(Cc2cn(CC(O)CN3CCOCC3)nn2)[C@@H](C)C1] | 191 |
| 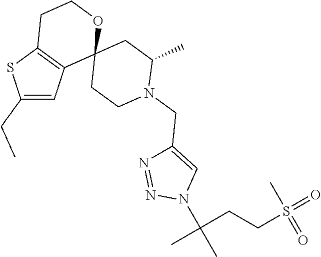 [SMILES: CCc1cc2c(s1)CCO[C@@]21CCN(Cc2cn(C(C)(C)CCS(C)(=O)=O)nn2)[C@@H](C)C1] | 192 |
| 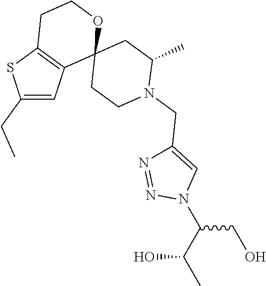 [SMILES: CCc1cc2c(s1)CCO[C@@]21CCN(Cc2cn(C(CO)[C@H](C)O)nn2)[C@@H](C)C1] | 193 |
| 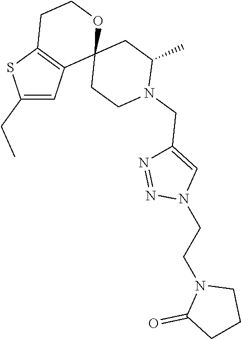 [SMILES: CCc1cc2c(s1)CCO[C@@]21CCN(Cc2cn(CCN3CCCC3=O)nn2)[C@@H](C)C1] | 194 |
| 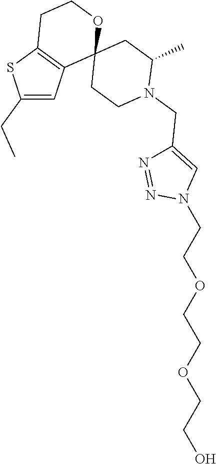 [SMILES: CCc1cc2c(s1)CCO[C@@]21CCN(Cc2cn(CCOCCOCCO)nn2)[C@@H](C)C1] | 195 |
| 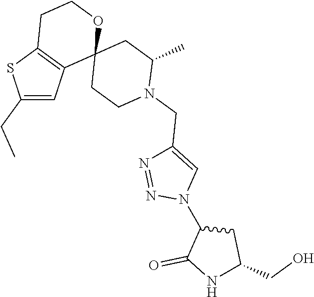 [SMILES: CCc1cc2c(s1)CCO[C@@]21CCN(Cc2cn(C3C[C@H](CO)NC3=O)nn2)[C@@H](C)C1] | 196 |
| 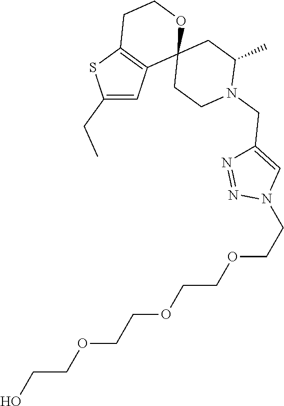 [SMILES: CCc1cc2c(s1)CCO[C@@]21CCN(Cc2cn(CCOCCOCCOCCO)nn2)[C@@H](C)C1] | 197 |
| 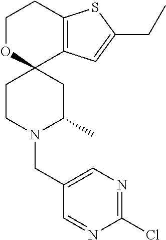 [SMILES: CCc1cc2c(s1)CCO[C@@]21CCN(Cc2cnc(Cl)nc2)[C@@H](C)C1] | 198 |
| 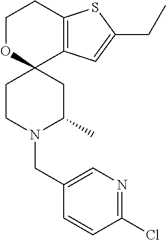 [SMILES: CCc1cc2c(s1)CCO[C@@]21CCN(Cc2ccc(Cl)nc2)[C@@H](C)C1] | 199 |
| 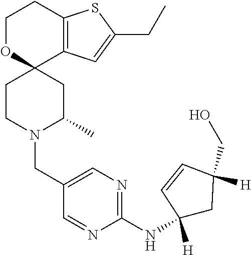 [SMILES: CCc1cc2c(s1)CCO[C@@]21CCN(Cc2cnc(N[C@@H]3C=C[C@H](CO)C3)nc2)[C@@H](C)C1] | 200 |
| 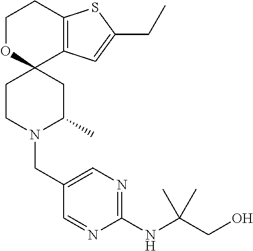 [SMILES: CCc1cc2c(s1)CCO[C@@]21CCN(Cc2cnc(NC(C)(C)CO)nc2)[C@@H](C)C1] | 201 |
| 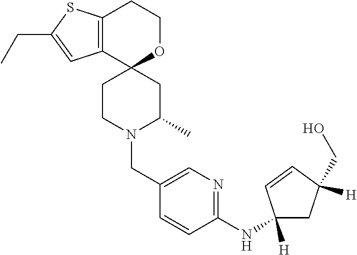 [SMILES: CCc1cc2c(s1)CCO[C@@]21CCN(Cc2ccc(N[C@@H]3C=C[C@H](CO)C3)nc2)[C@@H](C)C1] | 202 |
| 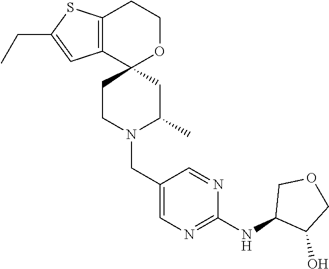 [SMILES: CCc1cc2c(s1)CCO[C@@]21CCN(Cc2cnc(N[C@H]3COC[C@@H]3O)nc2)[C@@H](C)C1] | 203 |
| 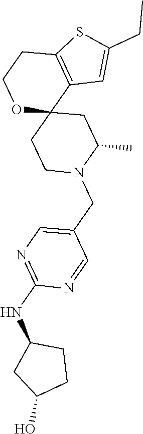 [SMILES: CCc1cc2c(s1)CCO[C@@]21CCN(Cc2cnc(N[C@H]3CC[C@H](O)C3)nc2)[C@@H](C)C1] | 204 |
| 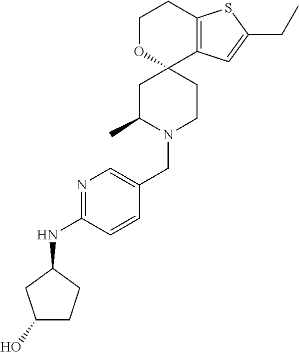 [SMILES: CCc1cc2c(s1)CCO[C@@]21CCN(Cc2ccc(N[C@H]3CC[C@H](O)C3)nc2)[C@@H](C)C1] | 205 |
| 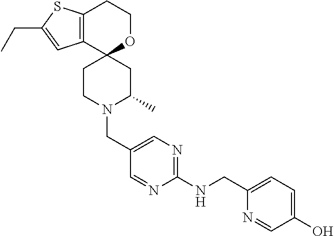 [SMILES: CCc1cc2c(s1)CCO[C@@]21CCN(Cc2cnc(NCc3ccc(O)cn3)nc2)[C@@H](C)C1] | 206 |
| 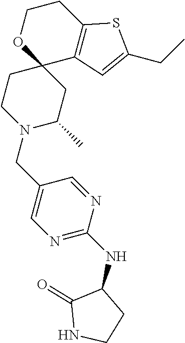 [SMILES: CCc1cc2c(s1)CCO[C@@]21CCN(Cc2cnc(N[C@H]3CCNC3=O)nc2)[C@@H](C)C1] | 207 |
| 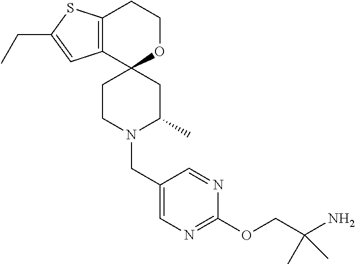 [SMILES: CCc1cc2c(s1)CCO[C@@]21CCN(Cc2cnc(OCC(C)(C)N)nc2)[C@@H](C)C1] | 208 |
| 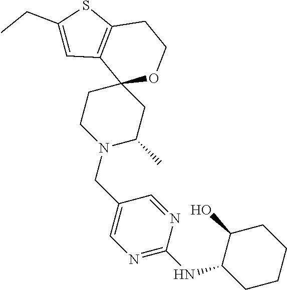 [SMILES: CCc1cc2c(s1)CCO[C@@]21CCN(Cc2cnc(N[C@H]3CCCC[C@@H]3O)nc2)[C@@H](C)C1] | 209 |
| 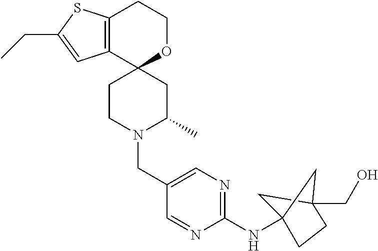 [SMILES: CCc1cc2c(s1)CCO[C@@]21CCN(Cc2cnc(NC34CCC(CO)(C3)C4)nc2)[C@@H](C)C1] | 210 |
| 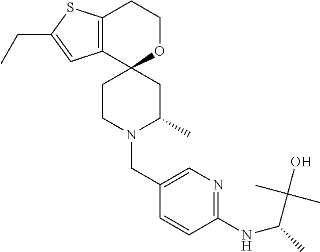 [SMILES: CCc1cc2c(s1)CCO[C@@]21CCN(Cc2ccc(N[C@@H](C)C(C)(C)O)nc2)[C@@H](C)C1] | 211 |
| 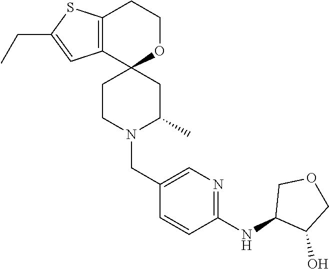 [SMILES: CCc1cc2c(s1)CCO[C@@]21CCN(Cc2ccc(N[C@H]3COC[C@@H]3O)nc2)[C@@H](C)C1] | 212 |
| 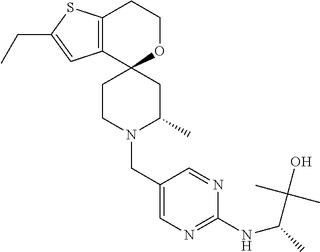 [SMILES: CCc1cc2c(s1)CCO[C@@]21CCN(Cc2cnc(N[C@@H](C)C(C)(C)O)nc2)[C@@H](C)C1] | 213 |
| 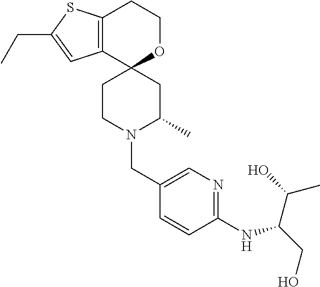 [SMILES: CCc1cc2c(s1)CCO[C@@]21CCN(Cc2ccc(N[C@@H](CO)[C@@H](C)O)nc2)[C@@H](C)C1] | 214 |
| 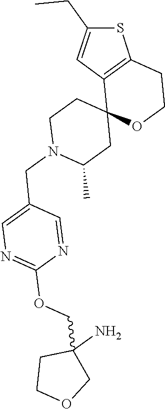 [SMILES: CCc1cc2c(s1)CCO[C@@]21CCN(Cc2cnc(OCC3(N)CCOC3)nc2)[C@@H](C)C1] | 215 |
| 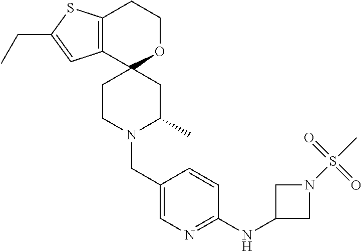 | 216 |
| 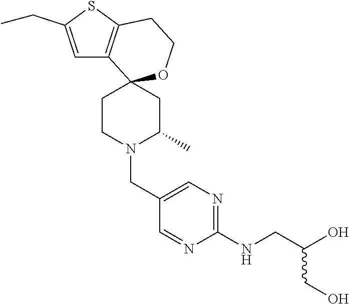 [SMILES: CCc1cc2c(s1)CCO[C@@]21CCN(Cc2cnc(NCC(O)CO)nc2)[C@@H](C)C1] | 217 |
| 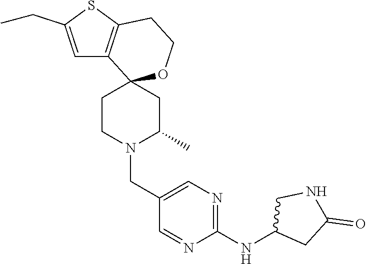 [SMILES: CCc1cc2c(s1)CCO[C@@]21CCN(Cc2cnc(NC3CNC(=O)C3)nc2)[C@@H](C)C1] | 218 |
| 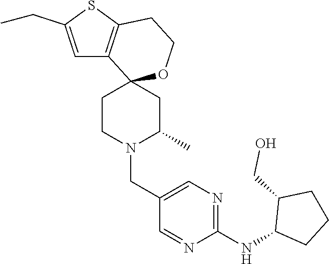 [SMILES: CCc1cc2c(s1)CCO[C@@]21CCN(Cc2cnc(N[C@H]3CCC[C@H]3CO)nc2)[C@@H](C)C1] | 219 |
| 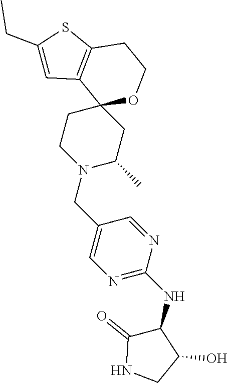 [SMILES: CCc1cc2c(s1)CCO[C@@]21CCN(Cc2cnc(N[C@@H]3C(=O)NC[C@H]3O)nc2)[C@@H](C)C1] | 220 |
| 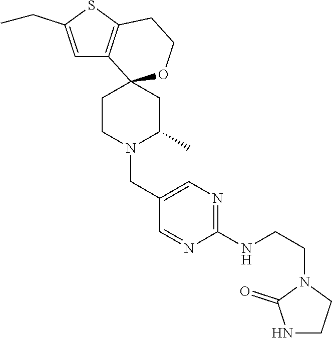 [SMILES: CCc1cc2c(s1)CCO[C@@]21CCN(Cc2cnc(NCCN3CCNC3=O)nc2)[C@@H](C)C1] | 221 |
| 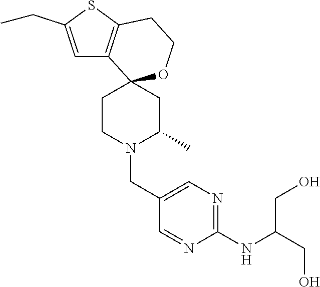 [SMILES: CCc1cc2c(s1)CCO[C@@]21CCN(Cc2cnc(NC(CO)CO)nc2)[C@@H](C)C1] | 222 |
| 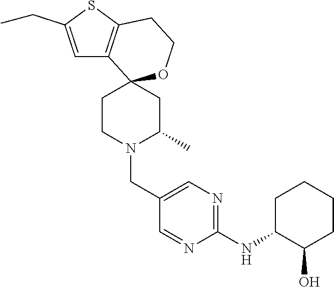 [SMILES: CCc1cc2c(s1)CCO[C@@]21CCN(Cc2cnc(N[C@@H]3CCCC[C@H]3O)nc2)[C@@H](C)C1] | 223 |
| 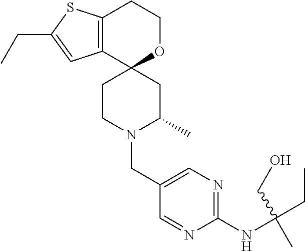 [SMILES: CCc1cc2c(s1)CCO[C@@]21CCN(Cc2cnc(NC(C)(CC)CO)nc2)[C@@H](C)C1] | 224 |
| 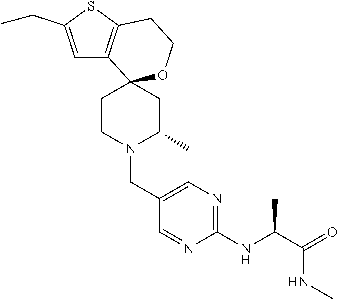 [SMILES: CCc1cc2c(s1)CCO[C@@]21CCN(Cc2cnc(N[C@@H](C)C(=O)NC)nc2)[C@@H](C)C1] | 225 |
| 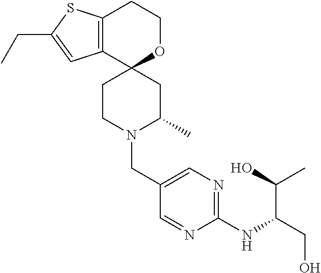 [SMILES: CCc1cc2c(s1)CCO[C@@]21CCN(Cc2cnc(N[C@@H](CO)[C@H](C)O)nc2)[C@@H](C)C1] | 226 |
| 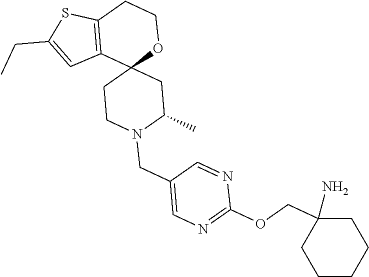 [SMILES: CCc1cc2c(s1)CCO[C@@]21CCN(Cc2cnc(OCC3(N)CCCCC3)nc2)[C@@H](C)C1] | 227 |
| 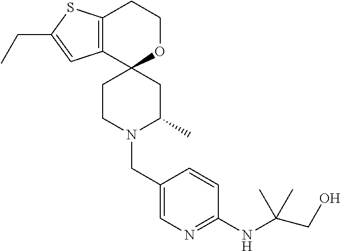 [SMILES: CCc1cc2c(s1)CCO[C@@]21CCN(Cc2ccc(NC(C)(C)CO)nc2)[C@@H](C)C1] | 228 |
| 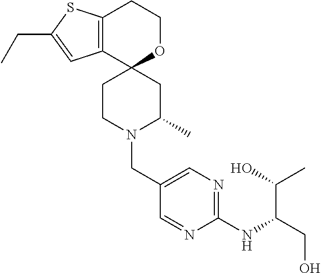 [SMILES: CCc1cc2c(s1)CCO[C@@]21CCN(Cc2cnc(N[C@@H](CO)[C@@H](C)O)nc2)[C@@H](C)C1] | 229 |
| 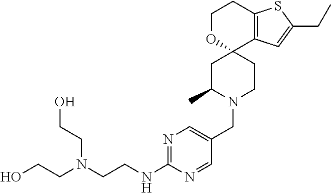 [SMILES: CCc1cc2c(s1)CCO[C@@]21CCN(Cc2cnc(NCCN(CCO)CCO)nc2)[C@@H](C)C1] | 230 |
| 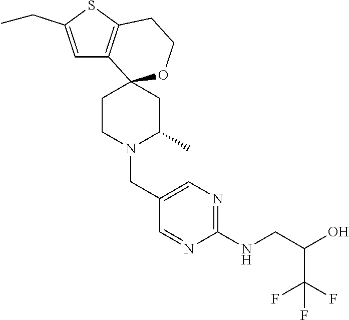 [SMILES: CCc1cc2c(s1)CCO[C@@]21CCN(Cc2cnc(NCC(O)C(F)(F)F)nc2)[C@@H](C)C1] | 231 |
| 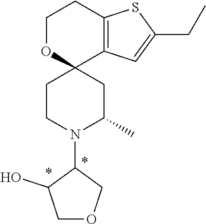 [SMILES: CCc1cc2c(s1)CCO[C@@]21CCN(C2COCC2O)[C@@H](C)C1] | 232 |
| 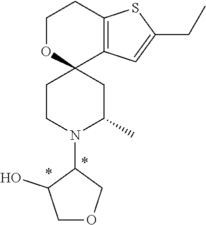 [SMILES: CCc1cc2c(s1)CCO[C@@]21CCN(C2COCC2O)[C@@H](C)C1] | 233 |
| 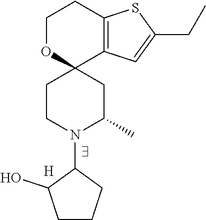 | 234 |
| 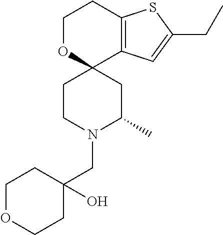 [SMILES: CCc1cc2c(s1)CCO[C@@]21CCN(CC2(O)CCOCC2)[C@@H](C)C1] | 235 |
| 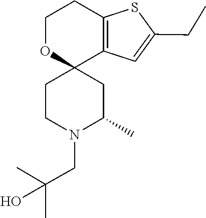 [SMILES: CCc1cc2c(s1)CCO[C@@]21CCN(CC(C)(C)O)[C@@H](C)C1] | 236 |
| 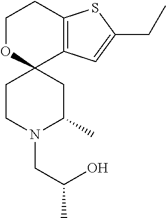 [SMILES: CCc1cc2c(s1)CCO[C@@]21CCN(C[C@@H](C)O)[C@@H](C)C1] | 237 |
| 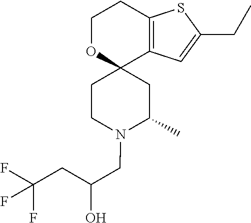 [SMILES: CCc1cc2c(s1)CCO[C@@]21CCN(CC(O)CC(F)(F)F)[C@@H](C)C1] | 238 |
| 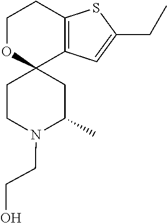 [SMILES: CCc1cc2c(s1)CCO[C@@]21CCN(CCO)[C@@H](C)C1] | 239 |
| 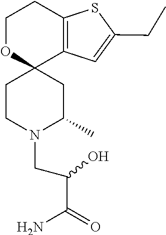 [SMILES: CCc1cc2c(s1)CCO[C@@]21CCN(CC(O)C(N)=O)[C@@H](C)C1] | 240 |
| 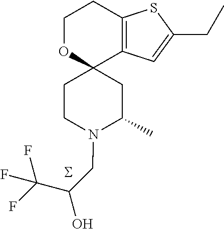 | 241 |
| 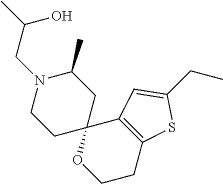 [SMILES: CCc1cc2c(s1)CCO[C@@]21CCN(CC(C)O)[C@@H](C)C1] | 242 |
| 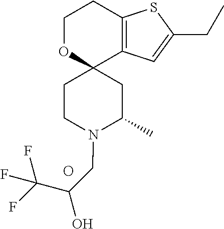 | 243 |
| 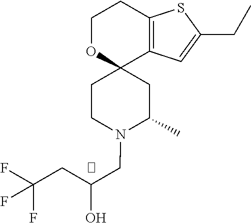 [SMILES: CCc1cc2c(s1)CCO[C@@]21CCN(CC(O)CC(F)(F)F)[C@@H](C)C1] | 244 |
| 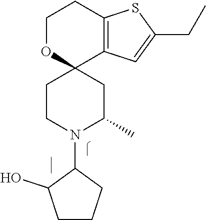 [SMILES: CCc1cc2c(s1)CCO[C@@]21CCN(C2CCCC2O)[C@@H](C)C1] | 245 |
| 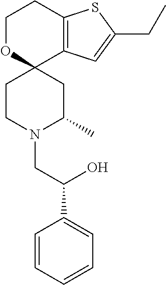 [SMILES: CCc1cc2c(s1)CCO[C@@]21CCN(C[C@H](O)c2ccccc2)[C@@H](C)C1] | 246 |
| 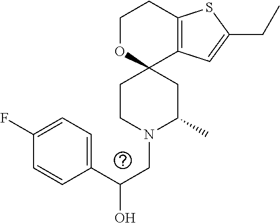 [SMILES: CCc1cc2c(s1)CCO[C@@]21CCN(CC(O)c2ccc(F)cc2)[C@@H](C)C1] | 247 |
| 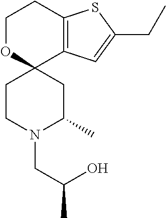 [SMILES: CCc1cc2c(s1)CCO[C@@]21CCN(C[C@H](C)O)[C@@H](C)C1] | 248 |
| 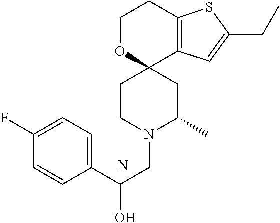 | 249 |
| 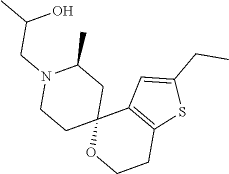 [SMILES: CCc1cc2c(s1)CCO[C@@]21CCN(CC(C)O)[C@@H](C)C1] | 250 |
| 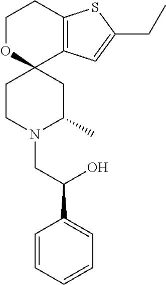 [SMILES: CCc1cc2c(s1)CCO[C@@]21CCN(C[C@@H](O)c2ccccc2)[C@@H](C)C1] | 251 |
| 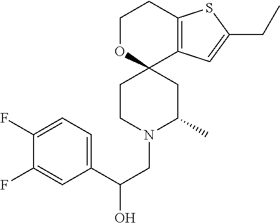 [SMILES: CCc1cc2c(s1)CCO[C@@]21CCN(CC(O)c2ccc(F)c(F)c2)[C@@H](C)C1] | 252 |
| 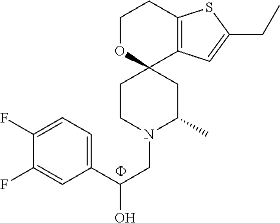 | 253 |
| 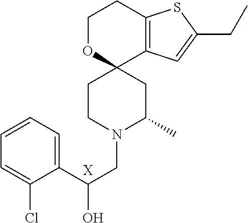 | 254 |
| 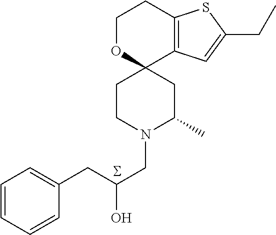 | 255 |
| 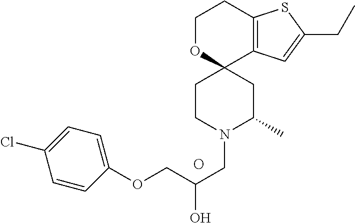 | 256 |
| 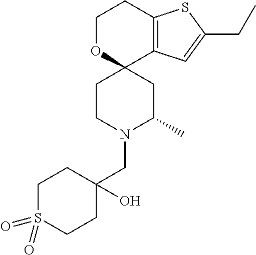 [SMILES: CCc1cc2c(s1)CCO[C@@]21CCN(CC2(O)CCS(=O)(=O)CC2)[C@@H](C)C1] | 257 |
| 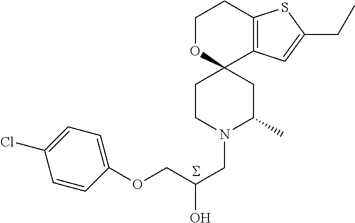 | 258 |
| 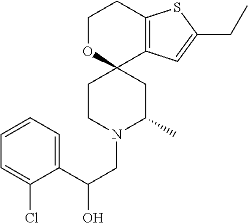 [SMILES: CCc1cc2c(s1)CCO[C@@]21CCN(CC(O)c2ccccc2Cl)[C@@H](C)C1] | 259 |
| 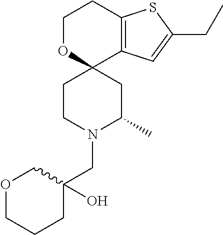 [SMILES: CCc1cc2c(s1)CCO[C@@]21CCN(CC2(O)CCCOC2)[C@@H](C)C1] | 260 |
| 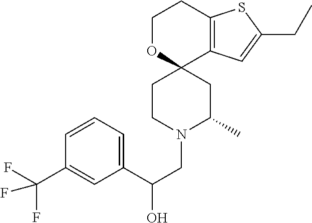 [SMILES: CCc1cc2c(s1)CCO[C@@]21CCN(CC(O)c2cccc(C(F)(F)F)c2)[C@@H](C)C1] | 261 |
| 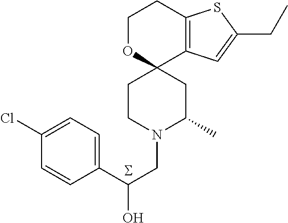 | 262 |
| 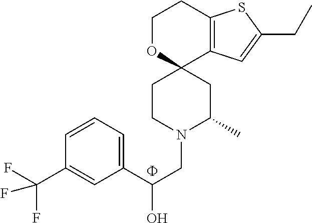 | 263 |
| 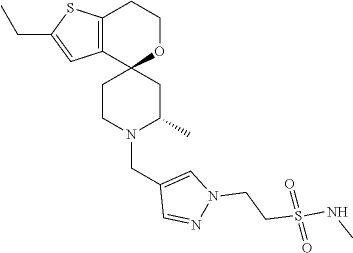 [SMILES: CCc1cc2c(s1)CCO[C@@]21CCN(Cc2cnn(CCS(=O)(=O)NC)c2)[C@@H](C)C1] | 264 |
| 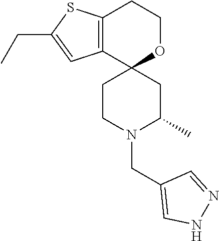 [SMILES: CCc1cc2c(s1)CCO[C@@]21CCN(Cc2cn[nH]c2)[C@@H](C)C1] | 265 |
| 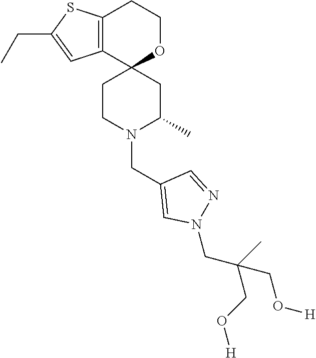 [SMILES: CCc1cc2c(s1)CCO[C@@]21CCN(Cc2cnn(CC(C)(CO)CO)c2)[C@@H](C)C1] | 266 |
| 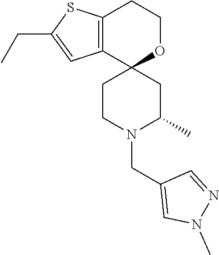 [SMILES: CCc1cc2c(s1)CCO[C@@]21CCN(Cc2cnn(C)c2)[C@@H](C)C1] | 267 |
| 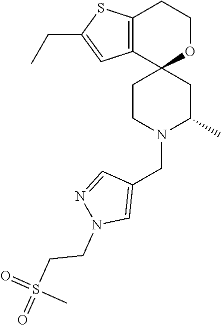 [SMILES: CCc1cc2c(s1)CCO[C@@]21CCN(Cc2cnn(CCS(C)(=O)=O)c2)[C@@H](C)C1] | 268 |
| 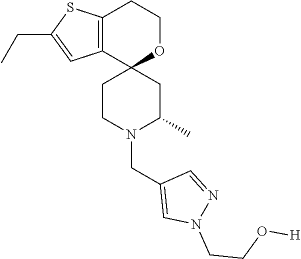 [SMILES: CCc1cc2c(s1)CCO[C@@]21CCN(Cc2cnn(CCO)c2)[C@@H](C)C1] | 269 |
| 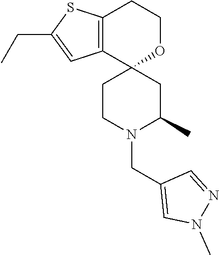 [SMILES: CCc1cc2c(s1)CCO[C@]21CCN(Cc2cnn(C)c2)[C@H](C)C1] | 270 |
| 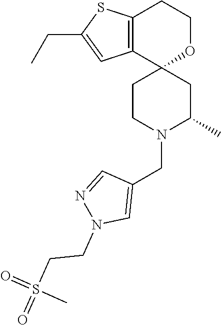 [SMILES: CCc1cc2c(s1)CCO[C@]21CCN(Cc2cnn(CCS(C)(=O)=O)c2)[C@@H](C)C1] | 271 |
| 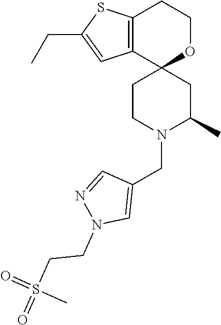 [SMILES: CCc1cc2c(s1)CCO[C@@]21CCN(Cc2cnn(CCS(C)(=O)=O)c2)[C@H](C)C1] | 272 |
| 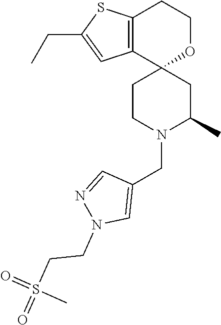 [SMILES: CCc1cc2c(s1)CCO[C@]21CCN(Cc2cnn(CCS(C)(=O)=O)c2)[C@H](C)C1] | 273 |
| 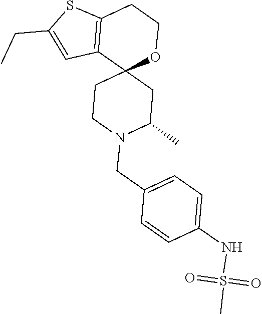 [SMILES: CCc1cc2c(s1)CCO[C@@]21CCN(Cc2ccc(NS(C)(=O)=O)cc2)[C@@H](C)C1] | 274 |
| 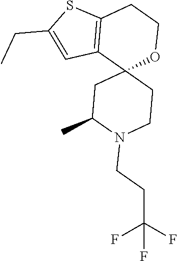 [SMILES: CCc1cc2c(s1)CCO[C@@]21CCN(CCC(F)(F)F)[C@@H](C)C1] | 275 |
| 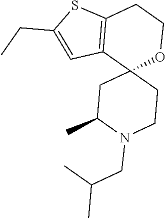 [SMILES: CCc1cc2c(s1)CCO[C@@]21CCN(CC(C)C)[C@@H](C)C1] | 276 |
| 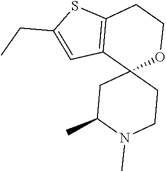 [SMILES: CCc1cc2c(s1)CCO[C@@]21CCN(C)[C@@H](C)C1] | 277 |
| 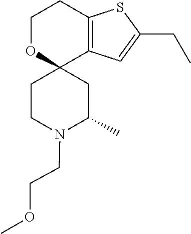 [SMILES: CCc1cc2c(s1)CCO[C@@]21CCN(CCOC)[C@@H](C)C1] | 278 |
| 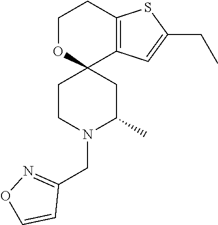 [SMILES: CCc1cc2c(s1)CCO[C@@]21CCN(Cc2ccon2)[C@@H](C)C1] | 279 |
| 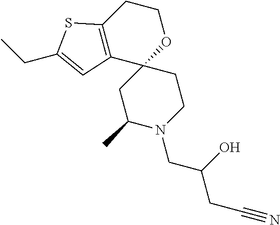 [SMILES: CCc1cc2c(s1)CCO[C@@]21CCN(CC(O)CC#N)[C@@H](C)C1] | 280 |
| 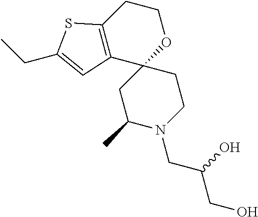 [SMILES: CCc1cc2c(s1)CCO[C@@]21CCN(CC(O)CO)[C@@H](C)C1] | 281 |
| 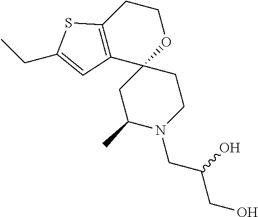 [SMILES: CCc1cc2c(s1)CCO[C@@]21CCN(CC(O)CO)[C@@H](C)C1] | 282 |
| 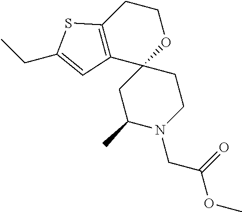 [SMILES: CCc1cc2c(s1)CCO[C@@]21CCN(CC(=O)OC)[C@@H](C)C1] | 283 |
| 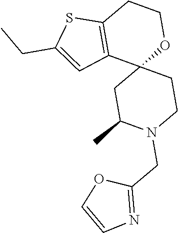 [SMILES: CCc1cc2c(s1)CCO[C@@]21CCN(Cc2ncco2)[C@@H](C)C1] | 284 |
| 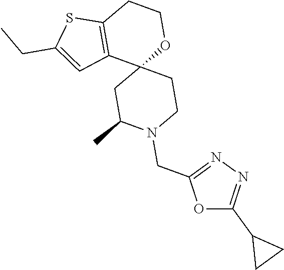 [SMILES: CCc1cc2c(s1)CCO[C@@]21CCN(Cc2nnc(C3CC3)o2)[C@@H](C)C1] | 285 |
| 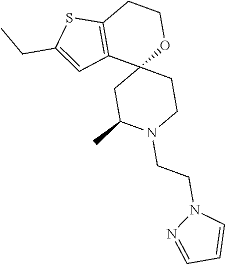 [SMILES: CCc1cc2c(s1)CCO[C@@]21CCN(CCn2cccn2)[C@@H](C)C1] | 286 |
| 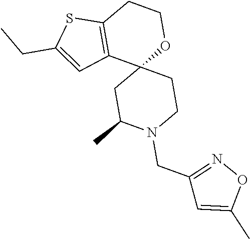 [SMILES: CCc1cc2c(s1)CCO[C@@]21CCN(Cc2cc(C)on2)[C@@H](C)C1] | 287 |
| 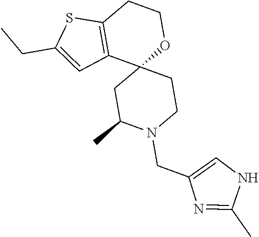 [SMILES: CCc1cc2c(s1)CCO[C@@]21CCN(Cc2c[nH]c(C)n2)[C@@H](C)C1] | 288 |
| 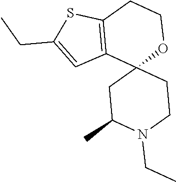 [SMILES: CCc1cc2c(s1)CCO[C@@]21CCN(CC)[C@@H](C)C1] | 289 |
| 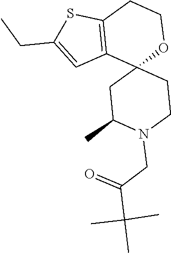 [SMILES: CCc1cc2c(s1)CCO[C@@]21CCN(CC(=O)C(C)(C)C)[C@@H](C)C1] | 290 |
| 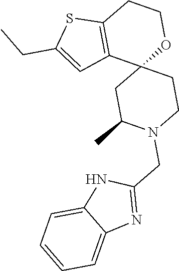 [SMILES: CCc1cc2c(s1)CCO[C@@]21CCN(Cc2nc3ccccc3[nH]2)[C@@H](C)C1] | 291 |
| 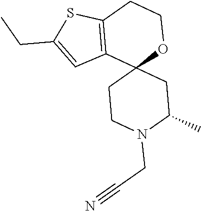 [SMILES: CCc1cc2c(s1)CCO[C@@]21CCN(CC#N)[C@@H](C)C1] | 292 |
| 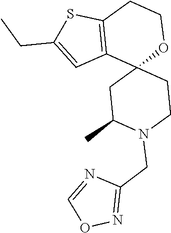 [SMILES: CCc1cc2c(s1)CCO[C@@]21CCN(Cc2ncon2)[C@@H](C)C1] | 293 |
| 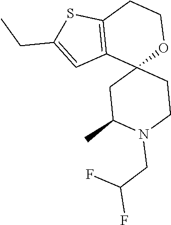 [SMILES: CCc1cc2c(s1)CCO[C@@]21CCN(CC(F)F)[C@@H](C)C1] | 294 |
| 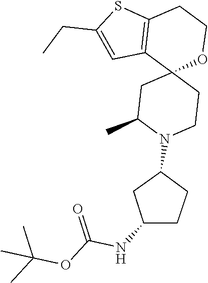 [SMILES: CCc1cc2c(s1)CCO[C@@]21CCN([C@@H]2CC[C@H](NC(=O)OC(C)(C)C)C2)[C@@H](C)C1] | 295 |
| 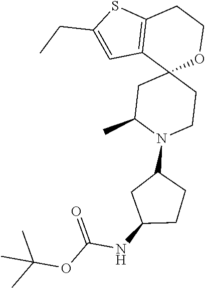 | 296 |
| 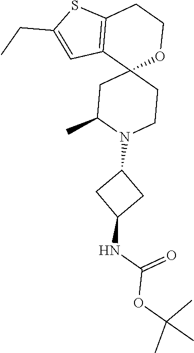 [SMILES: CCc1cc2c(s1)CCO[C@@]21CCN([C@H]2C[C@H](NC(=O)OC(C)(C)C)C2)[C@@H](C)C1] | 297 |
| 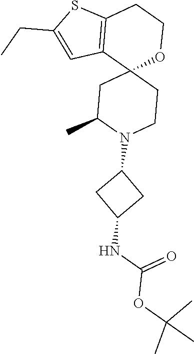 [SMILES: CCc1cc2c(s1)CCO[C@@]21CCN([C@H]2C[C@@H](NC(=O)OC(C)(C)C)C2)[C@@H](C)C1] | 298 |
| 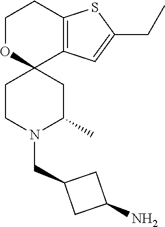 [SMILES: CCc1cc2c(s1)CCO[C@@]21CCN(C[C@H]2C[C@@H](N)C2)[C@@H](C)C1] | 299 |
| 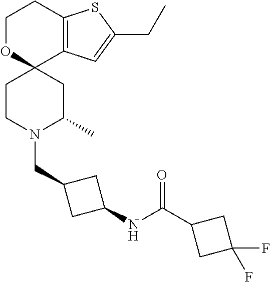 [SMILES: CCc1cc2c(s1)CCO[C@@]21CCN(C[C@H]2C[C@@H](NC(=O)C3CC(F)(F)C3)C2)[C@@H](C)C1] | 300 |
| 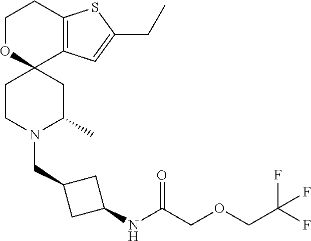 [SMILES: CCc1cc2c(s1)CCO[C@@]21CCN(C[C@H]2C[C@@H](NC(=O)COCC(F)(F)F)C2)[C@@H](C)C1] | 301 |
| 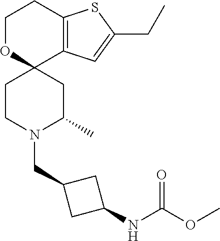 [SMILES: CCc1cc2c(s1)CCO[C@@]21CCN(C[C@H]2C[C@@H](NC(=O)OC)C2)[C@@H](C)C1] | 302 |
| 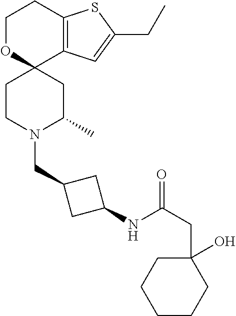 [SMILES: CCc1cc2c(s1)CCO[C@@]21CCN(C[C@H]2C[C@@H](NC(=O)CC3(O)CCCCC3)C2)[C@@H](C)C1] | 303 |
| 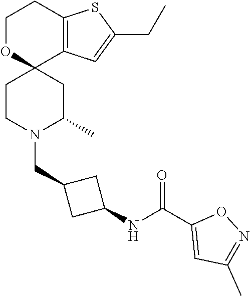 [SMILES: CCc1cc2c(s1)CCO[C@@]21CCN(C[C@H]2C[C@@H](NC(=O)c3cc(C)no3)C2)[C@@H](C)C1] | 304 |
| 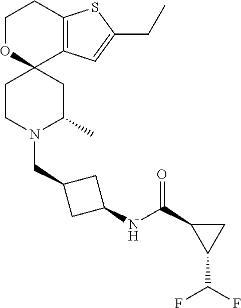 [SMILES: CCc1cc2c(s1)CCO[C@@]21CCN(C[C@H]2C[C@@H](NC(=O)[C@H]3C[C@@H]3C(F)F)C2)[C@@H](C)C1] | 305 |
| 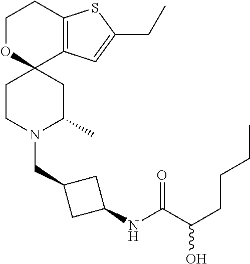 [SMILES: CCCCC(O)C(=O)N[C@H]1C[C@@H](CN2CC[C@]3(C[C@@H]2C)OCCc2sc(CC)cc23)C1] | 306 |
| 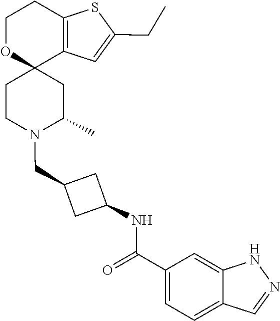 [SMILES: CCc1cc2c(s1)CCO[C@@]21CCN(C[C@H]2C[C@@H](NC(=O)c3ccc4cn[nH]c4c3)C2)[C@@H](C)C1] | 307 |
| 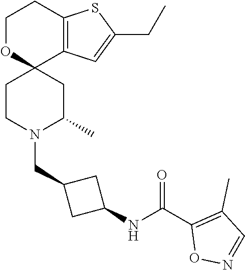 [SMILES: CCc1cc2c(s1)CCO[C@@]21CCN(C[C@H]2C[C@@H](NC(=O)c3oncc3C)C2)[C@@H](C)C1] | 308 |
| 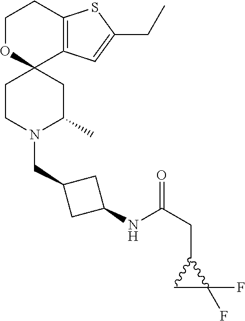 | 309 |
| 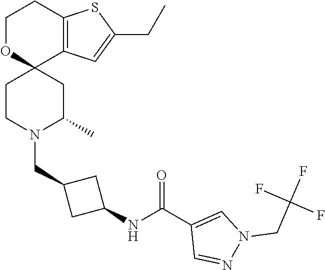 [SMILES: CCc1cc2c(s1)CCO[C@@]21CCN(C[C@H]2C[C@@H](NC(=O)c3cnn(CC(F)(F)F)c3)C2)[C@@H](C)C1] | 310 |
| 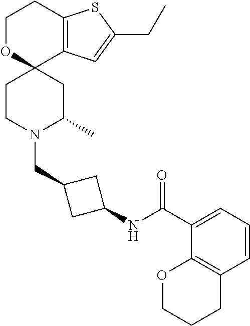 [SMILES: CCc1cc2c(s1)CCO[C@@]21CCN(C[C@H]2C[C@@H](NC(=O)c3cccc4c3OCCC4)C2)[C@@H](C)C1] | 311 |
| 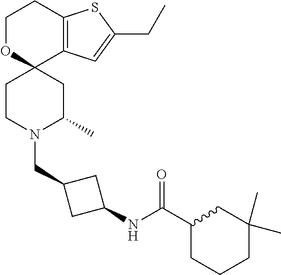 [SMILES: CCc1cc2c(s1)CCO[C@@]21CCN(C[C@H]2C[C@@H](NC(=O)C3CCCC(C)(C)C3)C2)[C@@H](C)C1] | 312 |
| 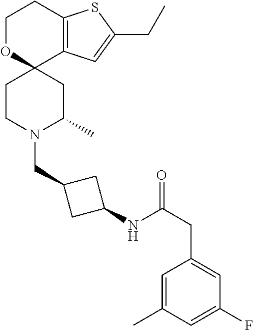 [SMILES: CCc1cc2c(s1)CCO[C@@]21CCN(C[C@H]2C[C@@H](NC(=O)Cc3cc(C)cc(F)c3)C2)[C@@H](C)C1] | 313 |
| 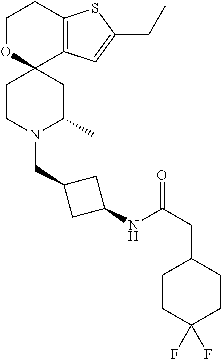 [SMILES: CCc1cc2c(s1)CCO[C@@]21CCN(C[C@H]2C[C@@H](NC(=O)CC3CCC(F)(F)CC3)C2)[C@@H](C)C1] | 314 |
| 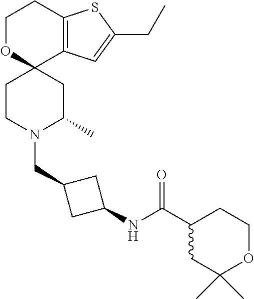 [SMILES: CCc1cc2c(s1)CCO[C@@]21CCN(C[C@H]2C[C@@H](NC(=O)C3CCOC(C)(C)C3)C2)[C@@H](C)C1] | 315 |
| 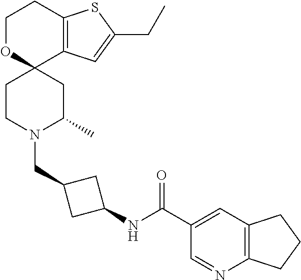 [SMILES: CCc1cc2c(s1)CCO[C@@]21CCN(C[C@H]2C[C@@H](NC(=O)c3cnc4c(c3)CCC4)C2)[C@@H](C)C1] | 316 |
| 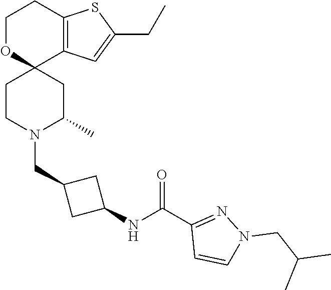 [SMILES: CCc1cc2c(s1)CCO[C@@]21CCN(C[C@H]2C[C@@H](NC(=O)c3ccn(CC(C)C)n3)C2)[C@@H](C)C1] | 317 |
| 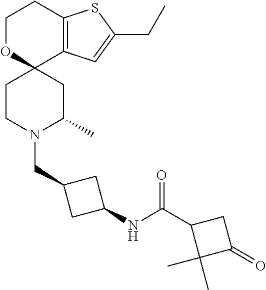 [SMILES: CCc1cc2c(s1)CCO[C@@]21CCN(C[C@H]2C[C@@H](NC(=O)C3CC(=O)C3(C)C)C2)[C@@H](C)C1] | 318 |
| 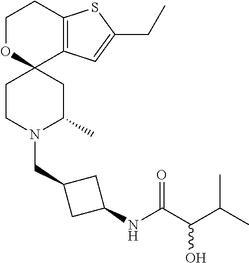 [SMILES: CCc1cc2c(s1)CCO[C@@]21CCN(C[C@H]2C[C@@H](NC(=O)C(O)C(C)C)C2)[C@@H](C)C1] | 319 |
| 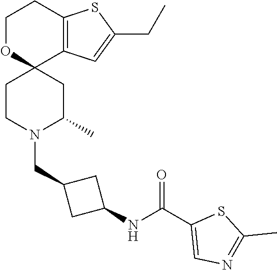 [SMILES: CCc1cc2c(s1)CCO[C@@]21CCN(C[C@H]2C[C@@H](NC(=O)c3cnc(C)s3)C2)[C@@H](C)C1] | 320 |
| 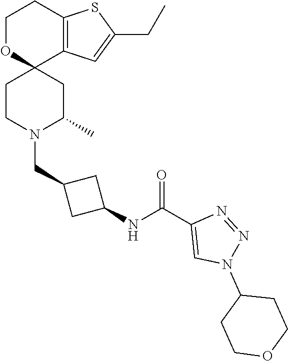 [SMILES: CCc1cc2c(s1)CCO[C@@]21CCN(C[C@H]2C[C@@H](NC(=O)c3cn(C4CCOCC4)nn3)C2)[C@@H](C)C1] | 321 |
| 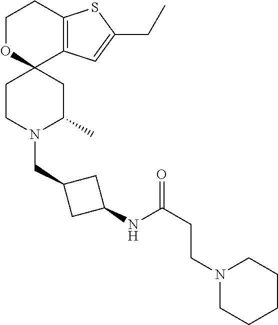 [SMILES: CCc1cc2c(s1)CCO[C@@]21CCN(C[C@H]2C[C@@H](NC(=O)CCN3CCCCC3)C2)[C@@H](C)C1] | 322 |
| 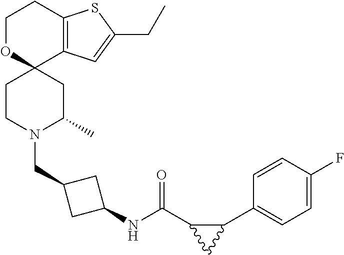 | 323 |
| 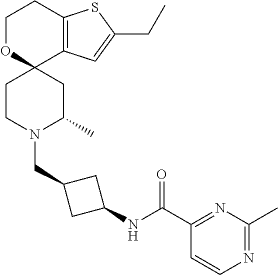 [SMILES: CCc1cc2c(s1)CCO[C@@]21CCN(C[C@H]2C[C@@H](NC(=O)c3ccnc(C)n3)C2)[C@@H](C)C1] | 324 |
| 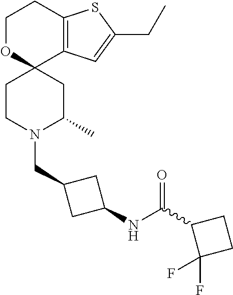 [SMILES: CCc1cc2c(s1)CCO[C@@]21CCN(C[C@H]2C[C@@H](NC(=O)C3CCC3(F)F)C2)[C@@H](C)C1] | 325 |
| 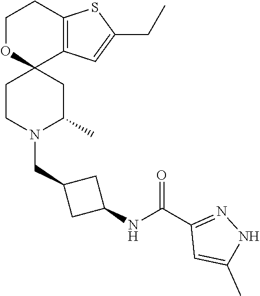 [SMILES: CCc1cc2c(s1)CCO[C@@]21CCN(C[C@H]2C[C@@H](NC(=O)c3cc(C)[nH]n3)C2)[C@@H](C)C1] | 326 |
| 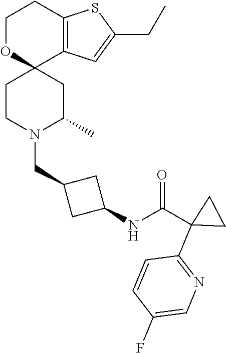 [SMILES: CCc1cc2c(s1)CCO[C@@]21CCN(C[C@H]2C[C@@H](NC(=O)C3(c4ccc(F)cn4)CC3)C2)[C@@H](C)C1] | 327 |
| 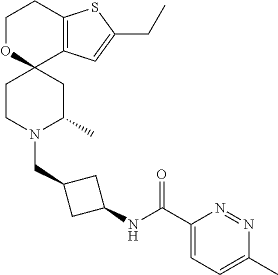 [SMILES: CCc1cc2c(s1)CCO[C@@]21CCN(C[C@H]2C[C@@H](NC(=O)c3ccc(C)nn3)C2)[C@@H](C)C1] | 328 |
| 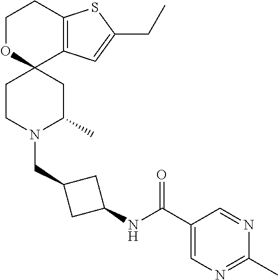 [SMILES: CCc1cc2c(s1)CCO[C@@]21CCN(C[C@H]2C[C@@H](NC(=O)c3cnc(C)nc3)C2)[C@@H](C)C1] | 329 |
| 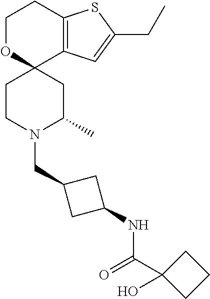 [SMILES: CCc1cc2c(s1)CCO[C@@]21CCN(C[C@H]2C[C@@H](NC(=O)C3(O)CCC3)C2)[C@@H](C)C1] | 330 |
| 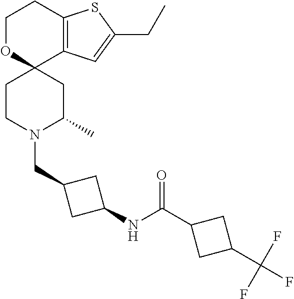 [SMILES: CCc1cc2c(s1)CCO[C@@]21CCN(C[C@H]2C[C@@H](NC(=O)C3CC(C(F)(F)F)C3)C2)[C@@H](C)C1] | 331 |
| 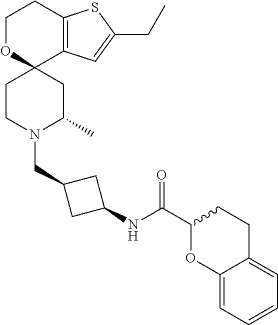 [SMILES: CCc1cc2c(s1)CCO[C@@]21CCN(C[C@H]2C[C@@H](NC(=O)C3CCc4ccccc4O3)C2)[C@@H](C)C1] | 332 |
| 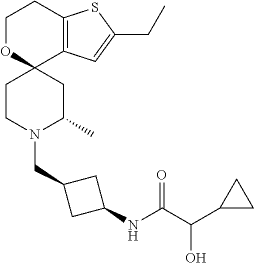 [SMILES: CCc1cc2c(s1)CCO[C@@]21CCN(C[C@H]2C[C@@H](NC(=O)C(O)C3CC3)C2)[C@@H](C)C1] | 333 |
| 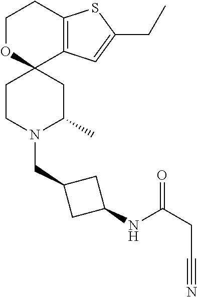 [SMILES: CCc1cc2c(s1)CCO[C@@]21CCN(C[C@H]2C[C@@H](NC(=O)CC#N)C2)[C@@H](C)C1] | 334 |
| 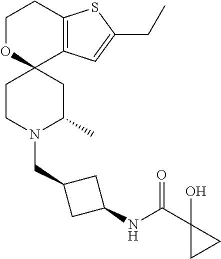 [SMILES: CCc1cc2c(s1)CCO[C@@]21CCN(C[C@H]2C[C@@H](NC(=O)C3(O)CC3)C2)[C@@H](C)C1] | 335 |
| 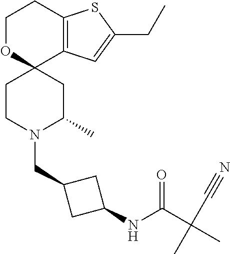 [SMILES: CCc1cc2c(s1)CCO[C@@]21CCN(C[C@H]2C[C@@H](NC(=O)C(C)(C)C#N)C2)[C@@H](C)C1] | 336 |
| 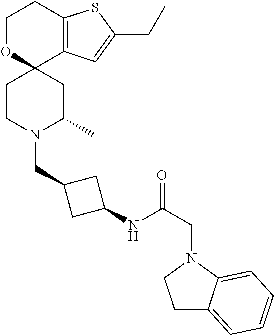 [SMILES: CCc1cc2c(s1)CCO[C@@]21CCN(C[C@H]2C[C@@H](NC(=O)CN3CCc4ccccc43)C2)[C@@H](C)C1] | 337 |
| 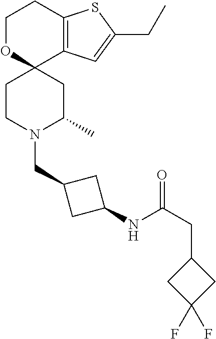 [SMILES: CCc1cc2c(s1)CCO[C@@]21CCN(C[C@H]2C[C@@H](NC(=O)CC3CC(F)(F)C3)C2)[C@@H](C)C1] | 338 |
| 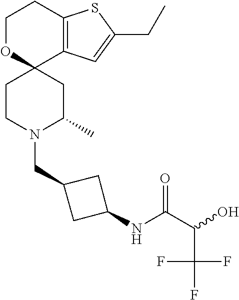 [SMILES: CCc1cc2c(s1)CCO[C@@]21CCN(C[C@H]2C[C@@H](NC(=O)C(O)C(F)(F)F)C2)[C@@H](C)C1] | 339 |
| 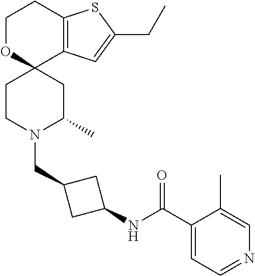 [SMILES: CCc1cc2c(s1)CCO[C@@]21CCN(C[C@H]2C[C@@H](NC(=O)c3ccncc3C)C2)[C@@H](C)C1] | 340 |
| 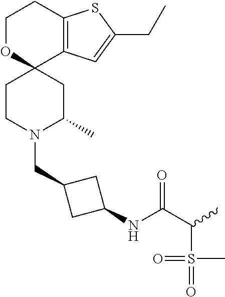 [SMILES: CCc1cc2c(s1)CCO[C@@]21CCN(C[C@H]2C[C@@H](NC(=O)C(C)S(C)(=O)=O)C2)[C@@H](C)C1] | 341 |
| 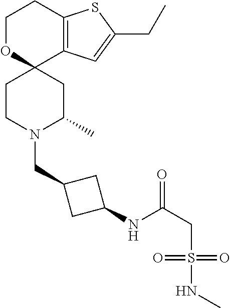 [SMILES: CCc1cc2c(s1)CCO[C@@]21CCN(C[C@H]2C[C@@H](NC(=O)CS(=O)(=O)NC)C2)[C@@H](C)C1] | 342 |
| 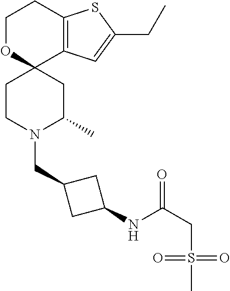 [SMILES: CCc1cc2c(s1)CCO[C@@]21CCN(C[C@H]2C[C@@H](NC(=O)CS(C)(=O)=O)C2)[C@@H](C)C1] | 343 |
| 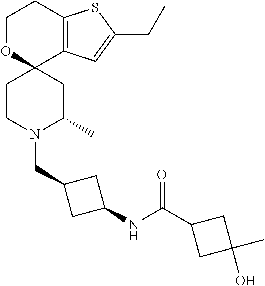 [SMILES: CCc1cc2c(s1)CCO[C@@]21CCN(C[C@H]2C[C@@H](NC(=O)C3CC(C)(O)C3)C2)[C@@H](C)C1] | 344 |
| 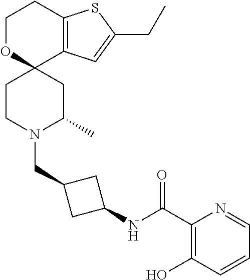 [SMILES: CCc1cc2c(s1)CCO[C@@]21CCN(C[C@H]2C[C@@H](NC(=O)c3ncccc3O)C2)[C@@H](C)C1] | 345 |
| 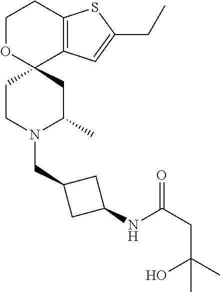 [SMILES: CCc1cc2c(s1)CCO[C@@]21CCN(C[C@H]2C[C@@H](NC(=O)CC(C)(C)O)C2)[C@@H](C)C1] | 346 |
| 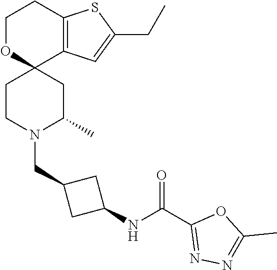 [SMILES: CCc1cc2c(s1)CCO[C@@]21CCN(C[C@H]2C[C@@H](NC(=O)c3nnc(C)o3)C2)[C@@H](C)C1] | 347 |
| 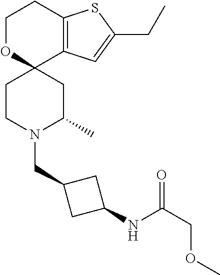 [SMILES: CCc1cc2c(s1)CCO[C@@]21CCN(C[C@H]2C[C@@H](NC(=O)COC)C2)[C@@H](C)C1] | 348 |
| 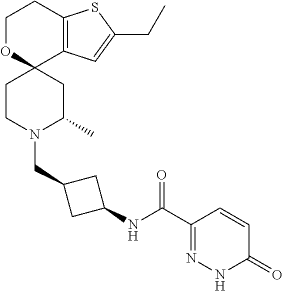 [SMILES: CCc1cc2c(s1)CCO[C@@]21CCN(C[C@H]2C[C@@H](NC(=O)c3ccc(=O)[nH]n3)C2)[C@@H](C)C1] | 349 |
| 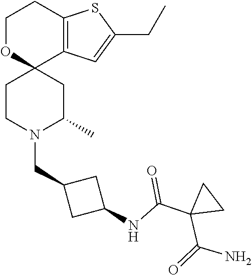 [SMILES: CCc1cc2c(s1)CCO[C@@]21CCN(C[C@H]2C[C@@H](NC(=O)C3(C(N)=O)CC3)C2)[C@@H](C)C1] | 350 |
| 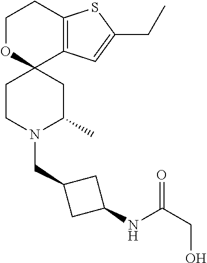 [SMILES: CCc1cc2c(s1)CCO[C@@]21CCN(C[C@H]2C[C@@H](NC(=O)CO)C2)[C@@H](C)C1] | 351 |
| 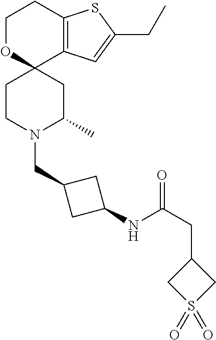 [SMILES: CCc1cc2c(s1)CCO[C@@]21CCN(C[C@H]2C[C@@H](NC(=O)CC3CS(=O)(=O)C3)C2)[C@@H](C)C1] | 352 |
| 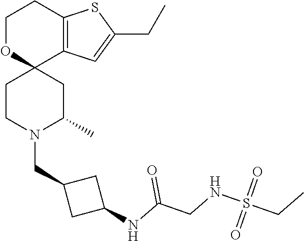 [SMILES: CCc1cc2c(s1)CCO[C@@]21CCN(C[C@H]2C[C@@H](NC(=O)CNS(=O)(=O)CC)C2)[C@@H](C)C1] | 353 |
| 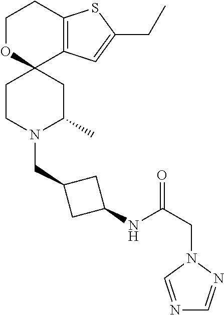 [SMILES: CCc1cc2c(s1)CCO[C@@]21CCN(C[C@H]2C[C@@H](NC(=O)Cn3cncn3)C2)[C@@H](C)C1] | 354 |
| 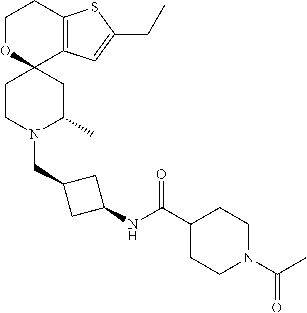 [SMILES: CCc1cc2c(s1)CCO[C@@]21CCN(C[C@H]2C[C@@H](NC(=O)C3CCN(C(C)=O)CC3)C2)[C@@H](C)C1] | 355 |
| 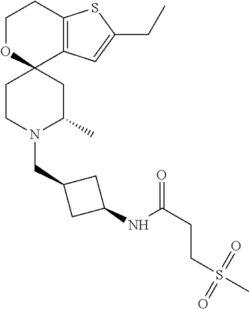 [SMILES: CCc1cc2c(s1)CCO[C@@]21CCN(C[C@H]2C[C@@H](NC(=O)CCS(C)(=O)=O)C2)[C@@H](C)C1] | 356 |
| 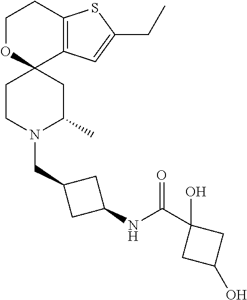 [SMILES: CCc1cc2c(s1)CCO[C@@]21CCN(C[C@H]2C[C@@H](NC(=O)C3(O)CC(O)C3)C2)[C@@H](C)C1] | 357 |
| 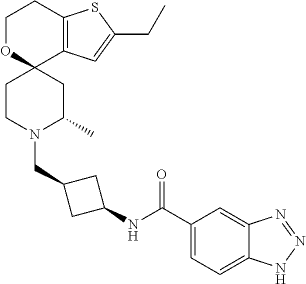 [SMILES: CCc1cc2c(s1)CCO[C@@]21CCN(C[C@H]2C[C@@H](NC(=O)c3ccc4[nH]nnc4c3)C2)[C@@H](C)C1] | 358 |
| 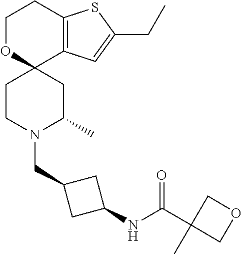 [SMILES: CCc1cc2c(s1)CCO[C@@]21CCN(C[C@H]2C[C@@H](NC(=O)C3(C)COC3)C2)[C@@H](C)C1] | 359 |
| 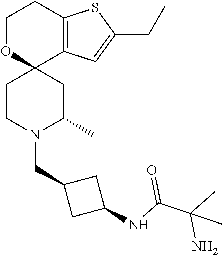 [SMILES: CCc1cc2c(s1)CCO[C@@]21CCN(C[C@H]2C[C@@H](NC(=O)C(C)(C)N)C2)[C@@H](C)C1] | 360 |
| 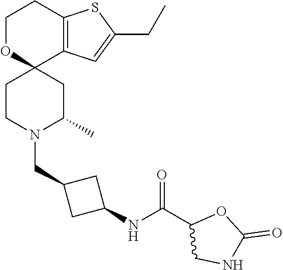 [SMILES: CCc1cc2c(s1)CCO[C@@]21CCN(C[C@H]2C[C@@H](NC(=O)C3CNC(=O)O3)C2)[C@@H](C)C1] | 361 |
| 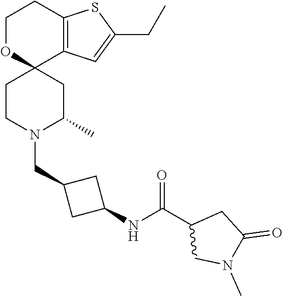 [SMILES: CCc1cc2c(s1)CCO[C@@]21CCN(C[C@H]2C[C@@H](NC(=O)C3CC(=O)N(C)C3)C2)[C@@H](C)C1] | 362 |
| 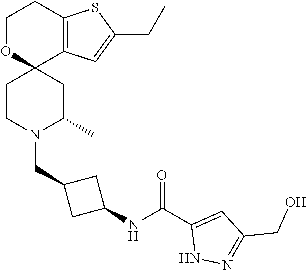 [SMILES: CCc1cc2c(s1)CCO[C@@]21CCN(C[C@H]2C[C@@H](NC(=O)c3cc(CO)n[nH]3)C2)[C@@H](C)C1] | 363 |
| 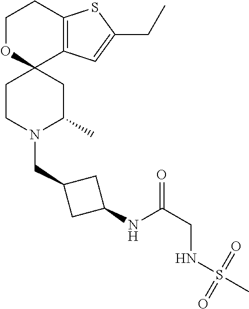 [SMILES: CCc1cc2c(s1)CCO[C@@]21CCN(C[C@H]2C[C@@H](NC(=O)CNS(C)(=O)=O)C2)[C@@H](C)C1] | 364 |
| 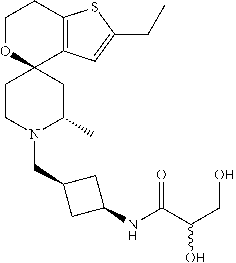 [SMILES: CCc1cc2c(s1)CCO[C@@]21CCN(C[C@H]2C[C@@H](NC(=O)C(O)CO)C2)[C@@H](C)C1] | 365 |
| 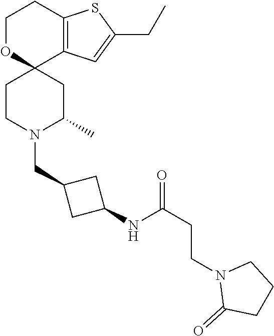 [SMILES: CCc1cc2c(s1)CCO[C@@]21CCN(C[C@H]2C[C@@H](NC(=O)CCN3CCCC3=O)C2)[C@@H](C)C1] | 366 |
| 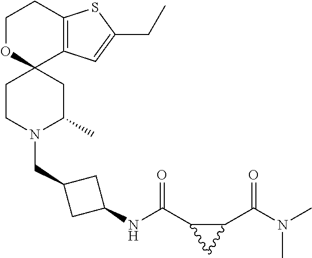 | 367 |
| 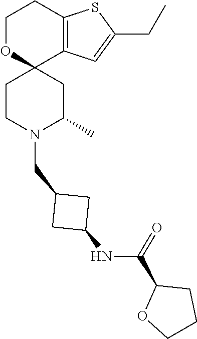 [SMILES: CCc1cc2c(s1)CCO[C@@]21CCN(C[C@H]2C[C@@H](NC(=O)[C@H]3CCCO3)C2)[C@@H](C)C1] | 368 |
| 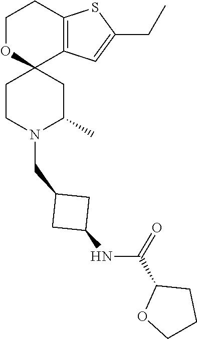 [SMILES: CCc1cc2c(s1)CCO[C@@]21CCN(C[C@H]2C[C@@H](NC(=O)[C@@H]3CCCO3)C2)[C@@H](C)C1] | 369 |
| 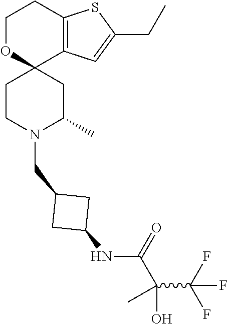 [SMILES: CCc1cc2c(s1)CCO[C@@]21CCN(C[C@H]2C[C@@H](NC(=O)C(C)(O)C(F)(F)F)C2)[C@@H](C)C1] | 370 |
| 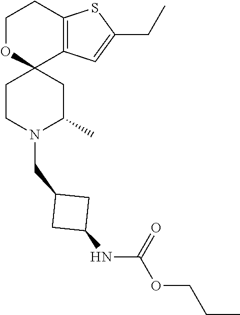 [SMILES: CCCOC(=O)N[C@H]1C[C@@H](CN2CC[C@]3(C[C@@H]2C)OCCc2sc(CC)cc23)C1] | 371 |
| 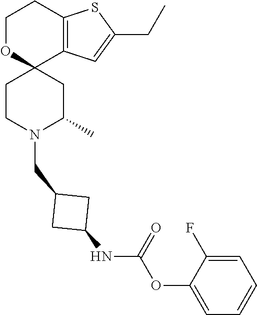 [SMILES: CCc1cc2c(s1)CCO[C@@]21CCN(C[C@H]2C[C@@H](NC(=O)Oc3ccccc3F)C2)[C@@H](C)C1] | 372 |
| 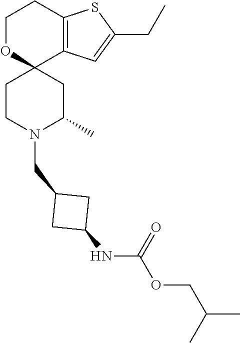 [SMILES: CCc1cc2c(s1)CCO[C@@]21CCN(C[C@H]2C[C@@H](NC(=O)OCC(C)C)C2)[C@@H](C)C1] | 373 |
| 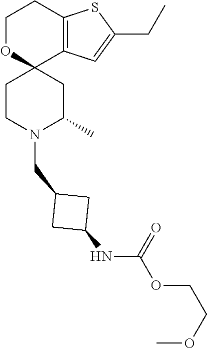 [SMILES: CCc1cc2c(s1)CCO[C@@]21CCN(C[C@H]2C[C@@H](NC(=O)OCCOC)C2)[C@@H](C)C1] | 374 |
| 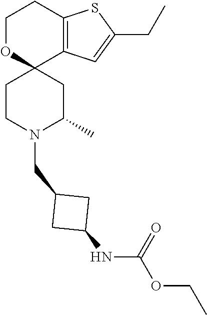 [SMILES: CCOC(=O)N[C@H]1C[C@@H](CN2CC[C@]3(C[C@@H]2C)OCCc2sc(CC)cc23)C1] | 375 |
| 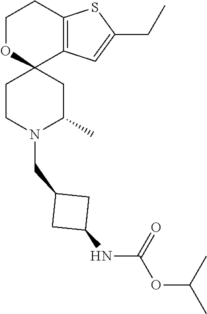 [SMILES: CCc1cc2c(s1)CCO[C@@]21CCN(C[C@H]2C[C@@H](NC(=O)OC(C)C)C2)[C@@H](C)C1] | 376 |
| 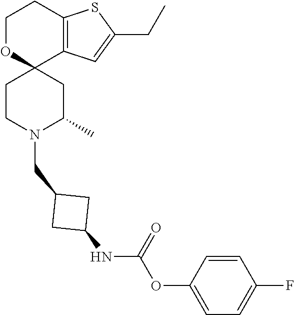 [SMILES: CCc1cc2c(s1)CCO[C@@]21CCN(C[C@H]2C[C@@H](NC(=O)Oc3ccc(F)cc3)C2)[C@@H](C)C1] | 377 |
| 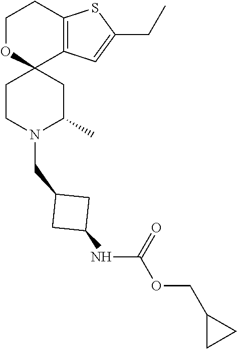 [SMILES: CCc1cc2c(s1)CCO[C@@]21CCN(C[C@H]2C[C@@H](NC(=O)OCC3CC3)C2)[C@@H](C)C1] | 378 |
| 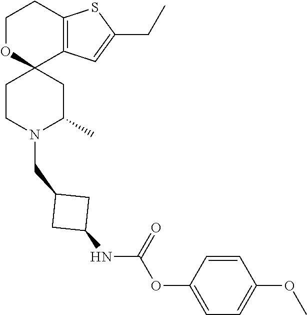 [SMILES: CCc1cc2c(s1)CCO[C@@]21CCN(C[C@H]2C[C@@H](NC(=O)Oc3ccc(OC)cc3)C2)[C@@H](C)C1] | 379 |
| 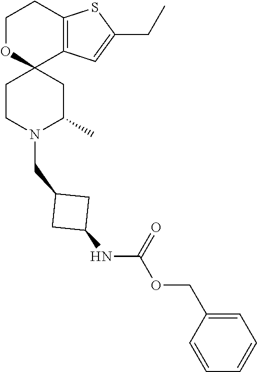 [SMILES: CCc1cc2c(s1)CCO[C@@]21CCN(C[C@H]2C[C@@H](NC(=O)OCc3ccccc3)C2)[C@@H](C)C1] | 380 |
| 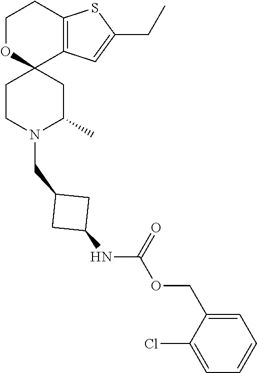 [SMILES: CCc1cc2c(s1)CCO[C@@]21CCN(C[C@H]2C[C@@H](NC(=O)OCc3ccccc3Cl)C2)[C@@H](C)C1] | 381 |
| 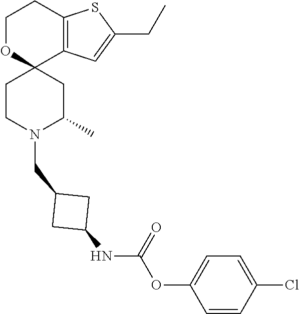 [SMILES: CCc1cc2c(s1)CCO[C@@]21CCN(C[C@H]2C[C@@H](NC(=O)Oc3ccc(Cl)cc3)C2)[C@@H](C)C1] | 382 |
| 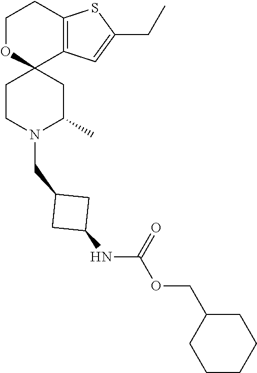 [SMILES: CCc1cc2c(s1)CCO[C@@]21CCN(C[C@H]2C[C@@H](NC(=O)OCC3CCCCC3)C2)[C@@H](C)C1] | 383 |
| 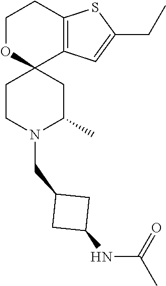 [SMILES: CCc1cc2c(s1)CCO[C@@]21CCN(C[C@H]2C[C@@H](NC(C)=O)C2)[C@@H](C)C1] | 384 |
| 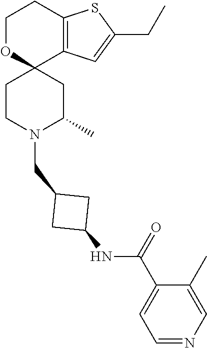 [SMILES: CCc1cc2c(s1)CCO[C@@]21CCN(C[C@H]2C[C@@H](NC(=O)c3ccncc3C)C2)[C@@H](C)C1] | 385 |
| 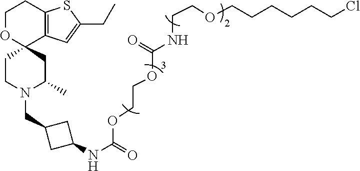 | 386 |
| 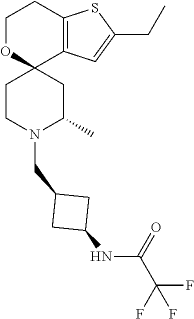 [SMILES: CCc1cc2c(s1)CCO[C@@]21CCN(C[C@H]2C[C@@H](NC(=O)C(F)(F)F)C2)[C@@H](C)C1] | 387 |
| 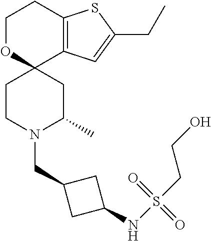 [SMILES: CCc1cc2c(s1)CCO[C@@]21CCN(C[C@H]2C[C@@H](NS(=O)(=O)CCO)C2)[C@@H](C)C1] | 388 |
| 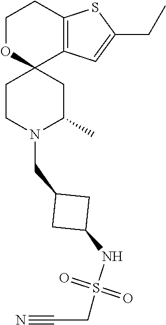 [SMILES: CCc1cc2c(s1)CCO[C@@]21CCN(C[C@H]2C[C@@H](NS(=O)(=O)CC#N)C2)[C@@H](C)C1] | 389 |
| 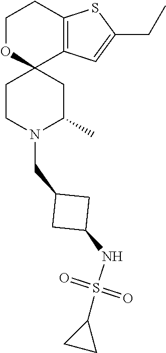 [SMILES: CCc1cc2c(s1)CCO[C@@]21CCN(C[C@H]2C[C@@H](NS(=O)(=O)C3CC3)C2)[C@@H](C)C1] | 390 |
| 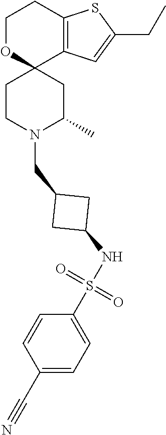 [SMILES: CCc1cc2c(s1)CCO[C@@]21CCN(C[C@H]2C[C@@H](NS(=O)(=O)c3ccc(C#N)cc3)C2)[C@@H](C)C1] | 391 |
| 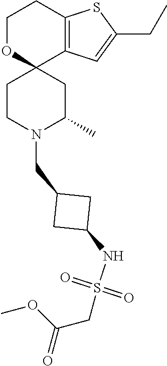 [SMILES: CCc1cc2c(s1)CCO[C@@]21CCN(C[C@H]2C[C@@H](NS(=O)(=O)CC(=O)OC)C2)[C@@H](C)C1] | 392 |
| 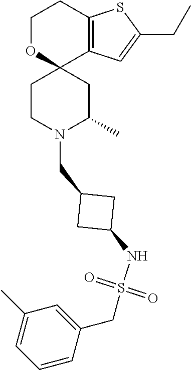 [SMILES: CCc1cc2c(s1)CCO[C@@]21CCN(C[C@H]2C[C@@H](NS(=O)(=O)Cc3cccc(C)c3)C2)[C@@H](C)C1] | 393 |
| 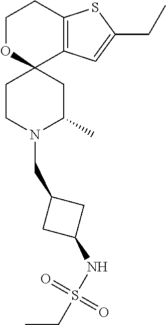 [SMILES: CCc1cc2c(s1)CCO[C@@]21CCN(C[C@H]2C[C@@H](NS(=O)(=O)CC)C2)[C@@H](C)C1] | 394 |
| 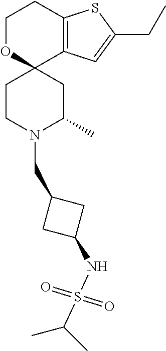 [SMILES: CCc1cc2c(s1)CCO[C@@]21CCN(C[C@H]2C[C@@H](NS(=O)(=O)C(C)C)C2)[C@@H](C)C1] | 395 |
| 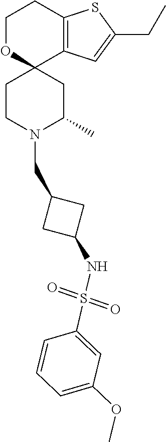 [SMILES: CCc1cc2c(s1)CCO[C@@]21CCN(C[C@H]2C[C@@H](NS(=O)(=O)c3cccc(OC)c3)C2)[C@@H](C)C1] | 396 |
| 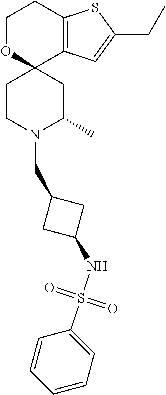 [SMILES: CCc1cc2c(s1)CCO[C@@]21CCN(C[C@H]2C[C@@H](NS(=O)(=O)c3ccccc3)C2)[C@@H](C)C1] | 397 |
| 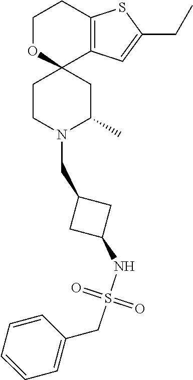 [SMILES: CCc1cc2c(s1)CCO[C@@]21CCN(C[C@H]2C[C@@H](NS(=O)(=O)Cc3ccccc3)C2)[C@@H](C)C1] | 398 |
| 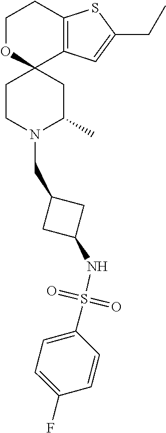 [SMILES: CCc1cc2c(s1)CCO[C@@]21CCN(C[C@H]2C[C@@H](NS(=O)(=O)c3ccc(F)cc3)C2)[C@@H](C)C1] | 399 |
| 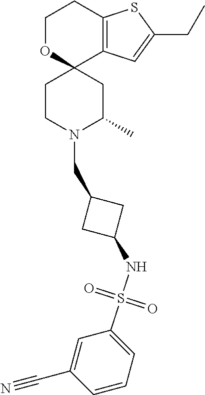 [SMILES: CCc1cc2c(s1)CCO[C@@]21CCN(C[C@H]2C[C@@H](NS(=O)(=O)c3cccc(C#N)c3)C2)[C@@H](C)C1] | 400 |
| 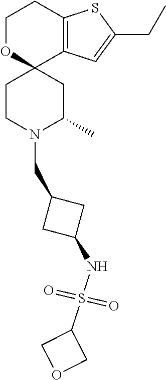 [SMILES: CCc1cc2c(s1)CCO[C@@]21CCN(C[C@H]2C[C@@H](NS(=O)(=O)C3COC3)C2)[C@@H](C)C1] | 401 |
| 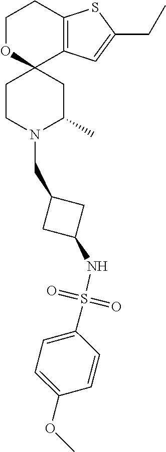 [SMILES: CCc1cc2c(s1)CCO[C@@]21CCN(C[C@H]2C[C@@H](NS(=O)(=O)c3ccc(OC)cc3)C2)[C@@H](C)C1] | 402 |
| 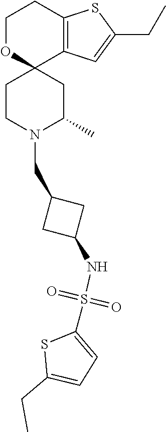 [SMILES: CCc1ccc(S(=O)(=O)N[C@H]2C[C@@H](CN3CC[C@]4(C[C@@H]3C)OCCc3sc(CC)cc34)C2)s1] | 403 |
| 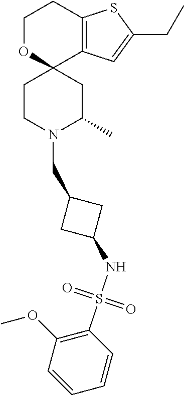 [SMILES: CCc1cc2c(s1)CCO[C@@]21CCN(C[C@H]2C[C@@H](NS(=O)(=O)c3ccccc3OC)C2)[C@@H](C)C1] | 404 |
| 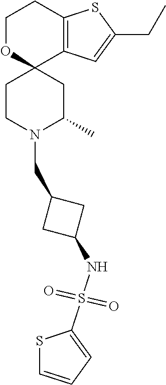 [SMILES: CCc1cc2c(s1)CCO[C@@]21CCN(C[C@H]2C[C@@H](NS(=O)(=O)c3cccs3)C2)[C@@H](C)C1] | 405 |
| 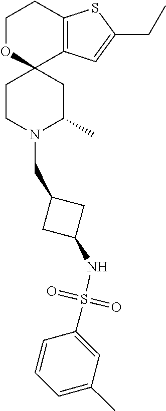 [SMILES: CCc1cc2c(s1)CCO[C@@]21CCN(C[C@H]2C[C@@H](NS(=O)(=O)c3cccc(C)c3)C2)[C@@H](C)C1] | 406 |
| 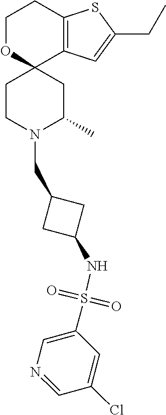 [SMILES: CCc1cc2c(s1)CCO[C@@]21CCN(C[C@H]2C[C@@H](NS(=O)(=O)c3cncc(Cl)c3)C2)[C@@H](C)C1] | 407 |
| 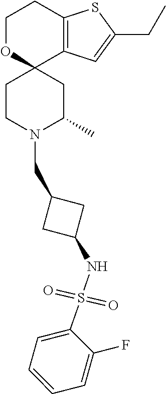 [SMILES: CCc1cc2c(s1)CCO[C@@]21CCN(C[C@H]2C[C@@H](NS(=O)(=O)c3ccccc3F)C2)[C@@H](C)C1] | 408 |
| 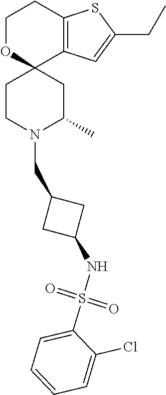 [SMILES: CCc1cc2c(s1)CCO[C@@]21CCN(C[C@H]2C[C@@H](NS(=O)(=O)c3ccccc3Cl)C2)[C@@H](C)C1] | 409 |
| 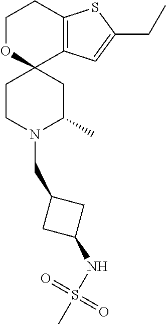 [SMILES: CCc1cc2c(s1)CCO[C@@]21CCN(C[C@H]2C[C@@H](NS(C)(=O)=O)C2)[C@@H](C)C1] | 410 |
| 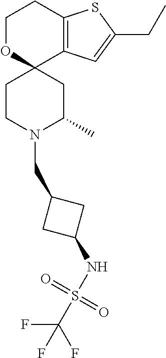 [SMILES: CCc1cc2c(s1)CCO[C@@]21CCN(C[C@H]2C[C@@H](NS(=O)(=O)C(F)(F)F)C2)[C@@H](C)C1] | 411 |
| 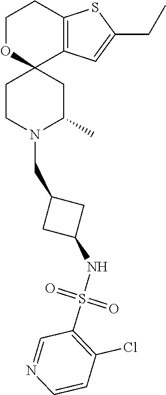 [SMILES: CCc1cc2c(s1)CCO[C@@]21CCN(C[C@H]2C[C@@H](NS(=O)(=O)c3cnccc3Cl)C2)[C@@H](C)C1] | 412 |
| 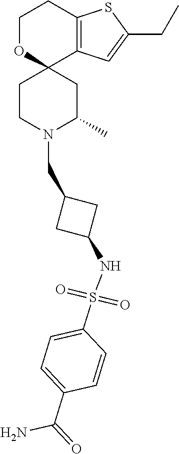 [SMILES: CCc1cc2c(s1)CCO[C@@]21CCN(C[C@H]2C[C@@H](NS(=O)(=O)c3ccc(C(N)=O)cc3)C2)[C@@H](C)C1] | 413 |
| 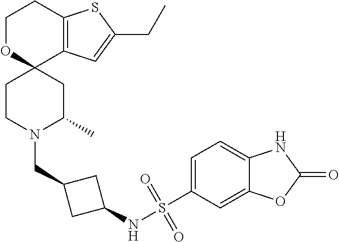 [SMILES: CCc1cc2c(s1)CCO[C@@]21CCN(C[C@H]2C[C@@H](NS(=O)(=O)c3ccc4[nH]c(=O)oc4c3)C2)[C@@H](C)C1] | 414 |
| 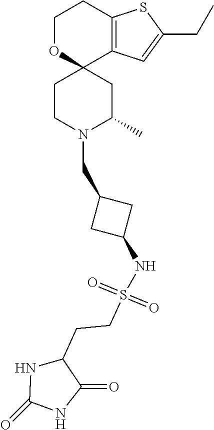 [SMILES: CCc1cc2c(s1)CCO[C@@]21CCN(C[C@H]2C[C@@H](NS(=O)(=O)CCC3NC(=O)NC3=O)C2)[C@@H](C)C1] | 415 |
| 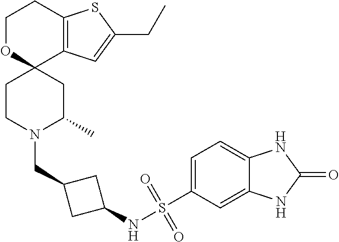 [SMILES: CCc1cc2c(s1)CCO[C@@]21CCN(C[C@H]2C[C@@H](NS(=O)(=O)c3ccc4[nH]c(=O)[nH]c4c3)C2)[C@@H](C)C1] | 416 |
| 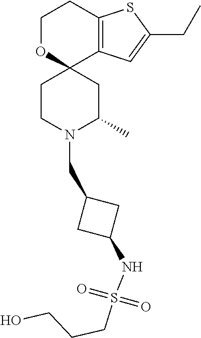 [SMILES: CCc1cc2c(s1)CCO[C@@]21CCN(C[C@H]2C[C@@H](NS(=O)(=O)CCCO)C2)[C@@H](C)C1] | 417 |
| 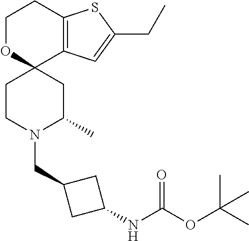 [SMILES: CCc1cc2c(s1)CCO[C@@]21CCN(C[C@H]2C[C@H](NC(=O)OC(C)(C)C)C2)[C@@H](C)C1] | 418 |
| 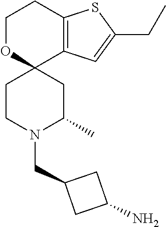 [SMILES: CCc1cc2c(s1)CCO[C@@]21CCN(C[C@H]2C[C@H](N)C2)[C@@H](C)C1] | 419 |
| 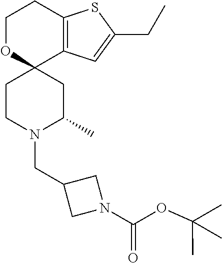 [SMILES: CCc1cc2c(s1)CCO[C@@]21CCN(CC2CN(C(=O)OC(C)(C)C)C2)[C@@H](C)C1] | 420 |
| 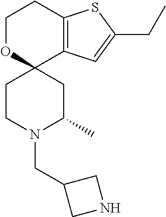 [SMILES: CCc1cc2c(s1)CCO[C@@]21CCN(CC2CNC2)[C@@H](C)C1] | 421 |
| 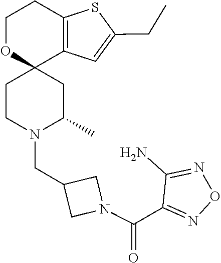 [SMILES: CCc1cc2c(s1)CCO[C@@]21CCN(CC2CN(C(=O)c3nonc3N)C2)[C@@H](C)C1] | 422 |
| 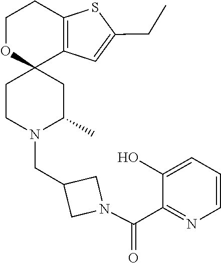 [SMILES: CCc1cc2c(s1)CCO[C@@]21CCN(CC2CN(C(=O)c3ncccc3O)C2)[C@@H](C)C1] | 423 |
| 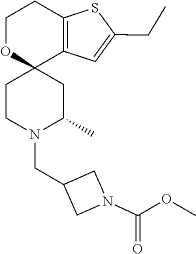 [SMILES: CCc1cc2c(s1)CCO[C@@]21CCN(CC2CN(C(=O)OC)C2)[C@@H](C)C1] | 424 |
| 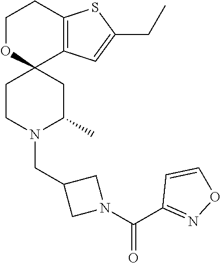 [SMILES: CCc1cc2c(s1)CCO[C@@]21CCN(CC2CN(C(=O)c3ccon3)C2)[C@@H](C)C1] | 425 |
| 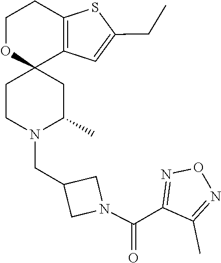 [SMILES: CCc1cc2c(s1)CCO[C@@]21CCN(CC2CN(C(=O)c3nonc3C)C2)[C@@H](C)C1] | 426 |
| 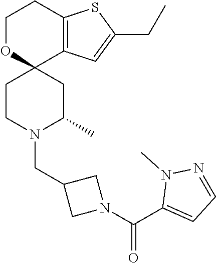 [SMILES: CCc1cc2c(s1)CCO[C@@]21CCN(CC2CN(C(=O)c3ccnn3C)C2)[C@@H](C)C1] | 427 |
| 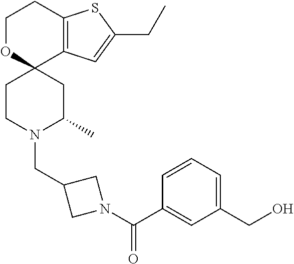 [SMILES: CCc1cc2c(s1)CCO[C@@]21CCN(CC2CN(C(=O)c3cccc(CO)c3)C2)[C@@H](C)C1] | 428 |
| 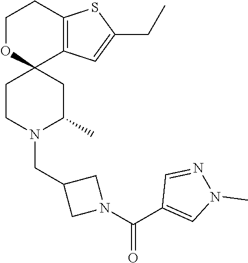 [SMILES: CCc1cc2c(s1)CCO[C@@]21CCN(CC2CN(C(=O)c3cnn(C)c3)C2)[C@@H](C)C1] | 429 |
| 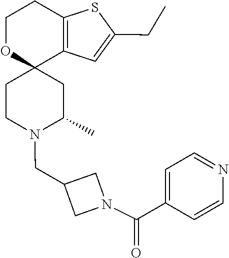 [SMILES: CCc1cc2c(s1)CCO[C@@]21CCN(CC2CN(C(=O)c3ccncc3)C2)[C@@H](C)C1] | 430 |
| 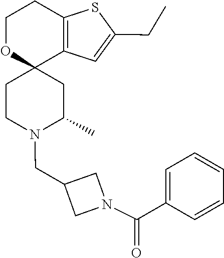 [SMILES: CCc1cc2c(s1)CCO[C@@]21CCN(CC2CN(C(=O)c3ccccc3)C2)[C@@H](C)C1] | 431 |
| 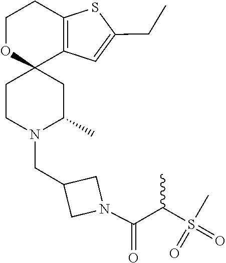 [SMILES: CCc1cc2c(s1)CCO[C@@]21CCN(CC2CN(C(=O)C(C)S(C)(=O)=O)C2)[C@@H](C)C1] | 432 |
| 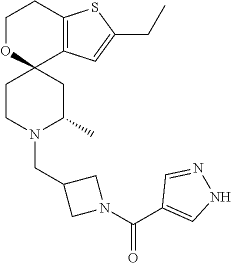 [SMILES: CCc1cc2c(s1)CCO[C@@]21CCN(CC2CN(C(=O)c3cn[nH]c3)C2)[C@@H](C)C1] | 433 |
| 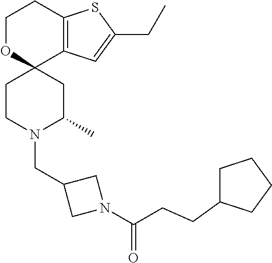 [SMILES: CCc1cc2c(s1)CCO[C@@]21CCN(CC2CN(C(=O)CCC3CCCC3)C2)[C@@H](C)C1] | 434 |
| 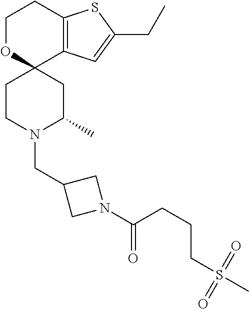 [SMILES: CCc1cc2c(s1)CCO[C@@]21CCN(CC2CN(C(=O)CCCS(C)(=O)=O)C2)[C@@H](C)C1] | 435 |
| 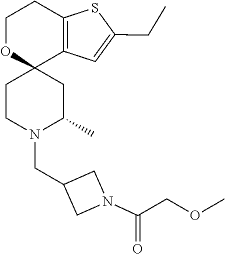 [SMILES: CCc1cc2c(s1)CCO[C@@]21CCN(CC2CN(C(=O)COC)C2)[C@@H](C)C1] | 436 |
| 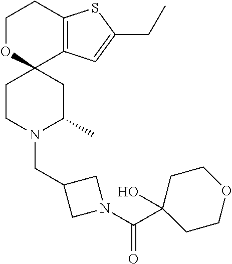 [SMILES: CCc1cc2c(s1)CCO[C@@]21CCN(CC2CN(C(=O)C3(O)CCOCC3)C2)[C@@H](C)C1] | 437 |
| 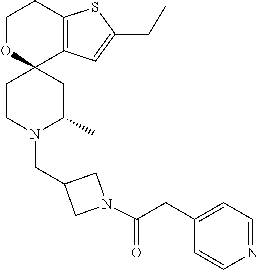 [SMILES: CCc1cc2c(s1)CCO[C@@]21CCN(CC2CN(C(=O)Cc3ccncc3)C2)[C@@H](C)C1] | 438 |
| 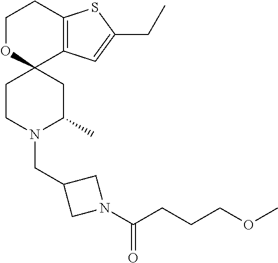 [SMILES: CCc1cc2c(s1)CCO[C@@]21CCN(CC2CN(C(=O)CCCOC)C2)[C@@H](C)C1] | 439 |
| 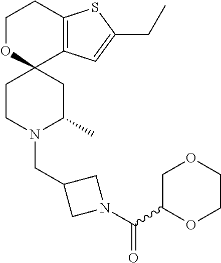 [SMILES: CCc1cc2c(s1)CCO[C@@]21CCN(CC2CN(C(=O)C3COCCO3)C2)[C@@H](C)C1] | 440 |
| 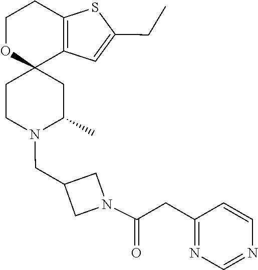 [SMILES: CCc1cc2c(s1)CCO[C@@]21CCN(CC2CN(C(=O)Cc3ccncn3)C2)[C@@H](C)C1] | 441 |
| 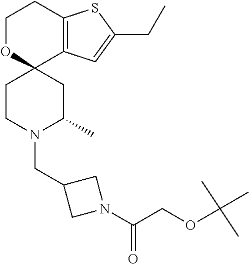 [SMILES: CCc1cc2c(s1)CCO[C@@]21CCN(CC2CN(C(=O)COC(C)(C)C)C2)[C@@H](C)C1] | 442 |
| 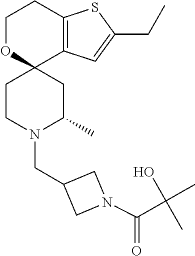 [SMILES: CCc1cc2c(s1)CCO[C@@]21CCN(CC2CN(C(=O)C(C)(C)O)C2)[C@@H](C)C1] | 443 |
| 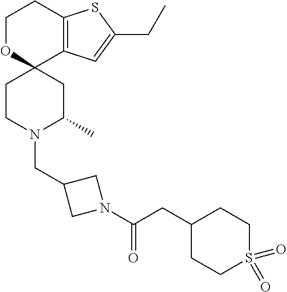 [SMILES: CCc1cc2c(s1)CCO[C@@]21CCN(CC2CN(C(=O)CC3CCS(=O)(=O)CC3)C2)[C@@H](C)C1] | 444 |
| 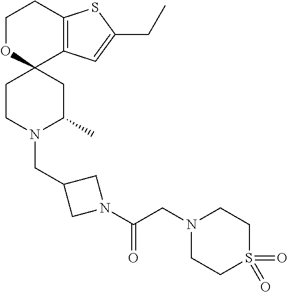 [SMILES: CCc1cc2c(s1)CCO[C@@]21CCN(CC2CN(C(=O)CN3CCS(=O)(=O)CC3)C2)[C@@H](C)C1] | 445 |
| 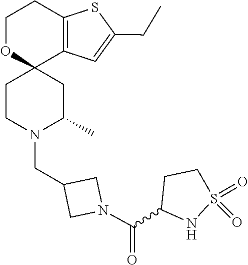 [SMILES: CCc1cc2c(s1)CCO[C@@]21CCN(CC2CN(C(=O)C3CCS(=O)(=O)N3)C2)[C@@H](C)C1] | 446 |
| 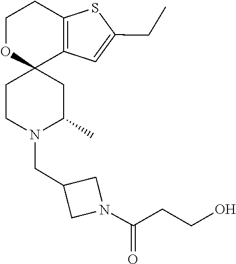 [SMILES: CCc1cc2c(s1)CCO[C@@]21CCN(CC2CN(C(=O)CCO)C2)[C@@H](C)C1] | 447 |
| 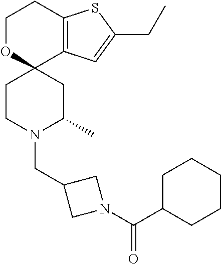 [SMILES: CCc1cc2c(s1)CCO[C@@]21CCN(CC2CN(C(=O)C3CCCCC3)C2)[C@@H](C)C1] | 448 |
| 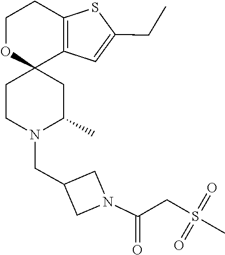 [SMILES: CCc1cc2c(s1)CCO[C@@]21CCN(CC2CN(C(=O)CS(C)(=O)=O)C2)[C@@H](C)C1] | 449 |
| 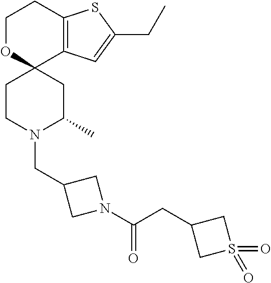 [SMILES: CCc1cc2c(s1)CCO[C@@]21CCN(CC2CN(C(=O)CC3CS(=O)(=O)C3)C2)[C@@H](C)C1] | 450 |
| 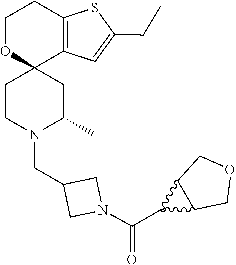 | 451 |
| 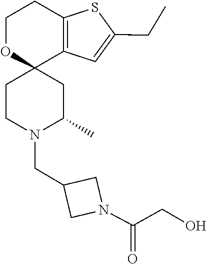 [SMILES: CCc1cc2c(s1)CCO[C@@]21CCN(CC2CN(C(=O)CO)C2)[C@@H](C)C1] | 452 |
| 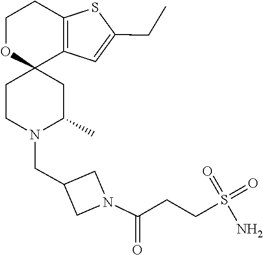 [SMILES: CCc1cc2c(s1)CCO[C@@]21CCN(CC2CN(C(=O)CCS(N)(=O)=O)C2)[C@@H](C)C1] | 453 |
| 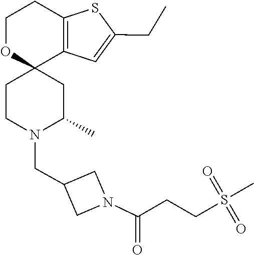 [SMILES: CCc1cc2c(s1)CCO[C@@]21CCN(CC2CN(C(=O)CCS(C)(=O)=O)C2)[C@@H](C)C1] | 454 |
| 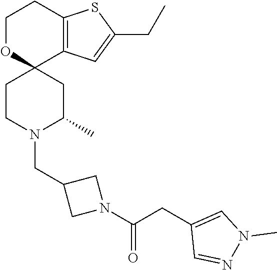 [SMILES: CCc1cc2c(s1)CCO[C@@]21CCN(CC2CN(C(=O)Cc3cnn(C)c3)C2)[C@@H](C)C1] | 455 |
| 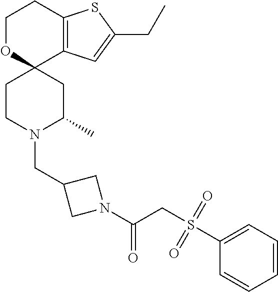 [SMILES: CCc1cc2c(s1)CCO[C@@]21CCN(CC2CN(C(=O)CS(=O)(=O)c3ccccc3)C2)[C@@H](C)C1] | 456 |
| 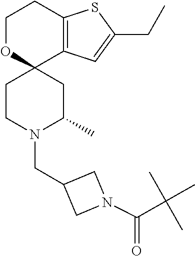 [SMILES: CCc1cc2c(s1)CCO[C@@]21CCN(CC2CN(C(=O)C(C)(C)C)C2)[C@@H](C)C1] | 457 |
| 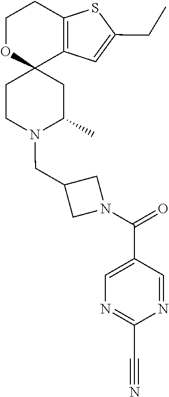 [SMILES: CCc1cc2c(s1)CCO[C@@]21CCN(CC2CN(C(=O)c3cnc(C#N)nc3)C2)[C@@H](C)C1] | 458 |
| 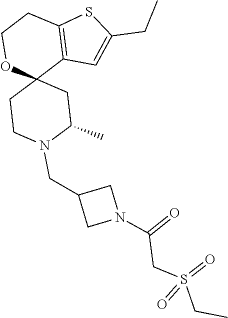 [SMILES: CCc1cc2c(s1)CCO[C@@]21CCN(CC2CN(C(=O)CS(=O)(=O)CC)C2)[C@@H](C)C1] | 459 |
| 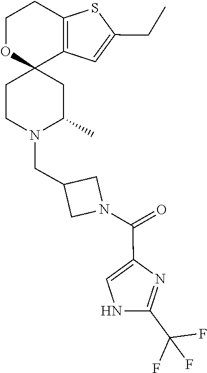 [SMILES: CCc1cc2c(s1)CCO[C@@]21CCN(CC2CN(C(=O)c3c[nH]c(C(F)(F)F)n3)C2)[C@@H](C)C1] | 460 |
| 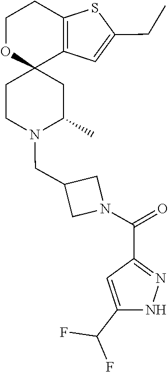 [SMILES: CCc1cc2c(s1)CCO[C@@]21CCN(CC2CN(C(=O)c3cc(C(F)F)[nH]n3)C2)[C@@H](C)C1] | 461 |
| 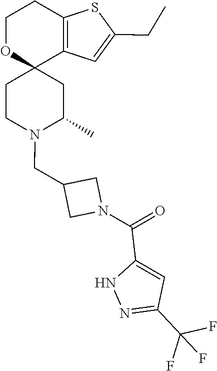 [SMILES: CCc1cc2c(s1)CCO[C@@]21CCN(CC2CN(C(=O)c3cc(C(F)(F)F)n[nH]3)C2)[C@@H](C)C1] | 462 |
| 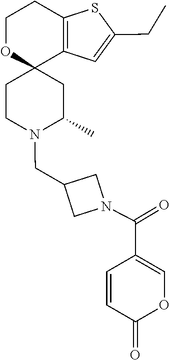 [SMILES: CCc1cc2c(s1)CCO[C@@]21CCN(CC2CN(C(=O)c3ccc(=O)oc3)C2)[C@@H](C)C1] | 463 |
| 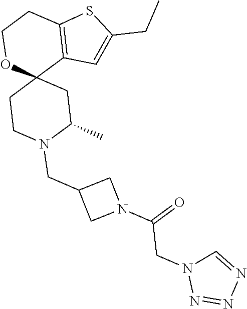 [SMILES: CCc1cc2c(s1)CCO[C@@]21CCN(CC2CN(C(=O)Cn3cnnn3)C2)[C@@H](C)C1] | 464 |
| 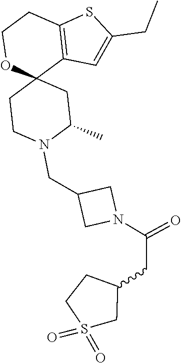 [SMILES: CCc1cc2c(s1)CCO[C@@]21CCN(CC2CN(C(=O)CC3CCS(=O)(=O)C3)C2)[C@@H](C)C1] | 465 |
| 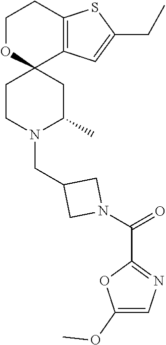 [SMILES: CCc1cc2c(s1)CCO[C@@]21CCN(CC2CN(C(=O)c3ncc(OC)o3)C2)[C@@H](C)C1] | 466 |
| 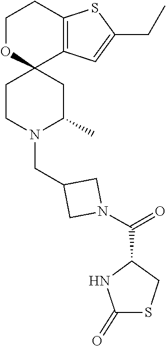 [SMILES: CCc1cc2c(s1)CCO[C@@]21CCN(CC2CN(C(=O)[C@@H]3CSC(=O)N3)C2)[C@@H](C)C1] | 467 |
| 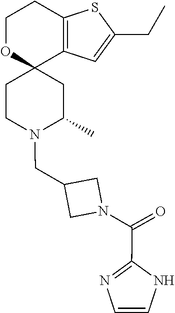 [SMILES: CCc1cc2c(s1)CCO[C@@]21CCN(CC2CN(C(=O)c3ncc[nH]3)C2)[C@@H](C)C1] | 468 |
| 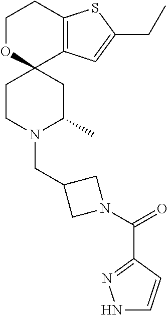 [SMILES: CCc1cc2c(s1)CCO[C@@]21CCN(CC2CN(C(=O)c3cc[nH]n3)C2)[C@@H](C)C1] | 469 |
| 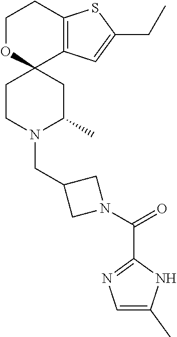 [SMILES: CCc1cc2c(s1)CCO[C@@]21CCN(CC2CN(C(=O)c3ncc(C)[nH]3)C2)[C@@H](C)C1] | 470 |
| 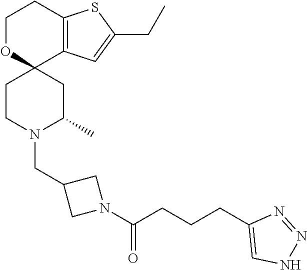 [SMILES: CCc1cc2c(s1)CCO[C@@]21CCN(CC2CN(C(=O)CCCc3c[nH]nn3)C2)[C@@H](C)C1] | 471 |
| 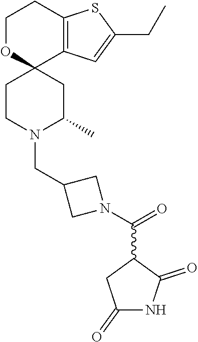 [SMILES: CCc1cc2c(s1)CCO[C@@]21CCN(CC2CN(C(=O)C3CC(=O)NC3=O)C2)[C@@H](C)C1] | 472 |
| 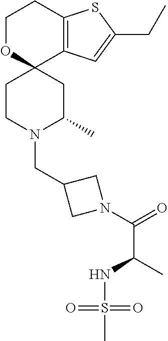 [SMILES: CCc1cc2c(s1)CCO[C@@]21CCN(CC2CN(C(=O)[C@@H](C)NS(C)(=O)=O)C2)[C@@H](C)C1] | 473 |
| 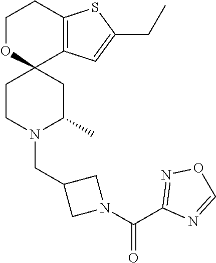 [SMILES: CCc1cc2c(s1)CCO[C@@]21CCN(CC2CN(C(=O)c3ncon3)C2)[C@@H](C)C1] | 474 |
| 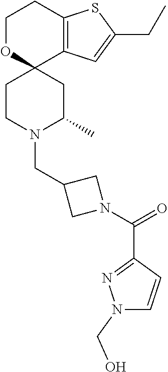 [SMILES: CCc1cc2c(s1)CCO[C@@]21CCN(CC2CN(C(=O)c3ccn(CO)n3)C2)[C@@H](C)C1] | 475 |
| 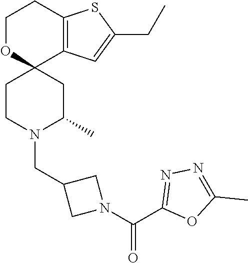 [SMILES: CCc1cc2c(s1)CCO[C@@]21CCN(CC2CN(C(=O)c3nnc(C)o3)C2)[C@@H](C)C1] | 476 |
| 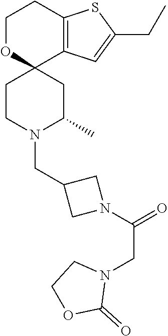 [SMILES: CCc1cc2c(s1)CCO[C@@]21CCN(CC2CN(C(=O)CN3CCOC3=O)C2)[C@@H](C)C1] | 477 |
| 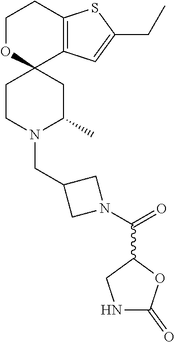 [SMILES: CCc1cc2c(s1)CCO[C@@]21CCN(CC2CN(C(=O)C3CNC(=O)O3)C2)[C@@H](C)C1] | 478 |
| 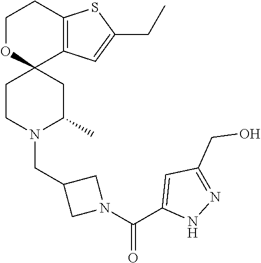 [SMILES: CCc1cc2c(s1)CCO[C@@]21CCN(CC2CN(C(=O)c3cc(CO)n[nH]3)C2)[C@@H](C)C1] | 479 |
| 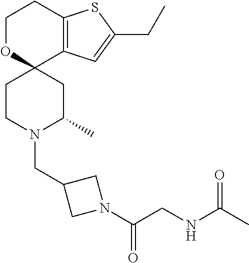 [SMILES: CCc1cc2c(s1)CCO[C@@]21CCN(CC2CN(C(=O)CNC(C)=O)C2)[C@@H](C)C1] | 480 |
| 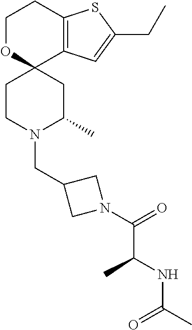 [SMILES: CCc1cc2c(s1)CCO[C@@]21CCN(CC2CN(C(=O)[C@H](C)NC(C)=O)C2)[C@@H](C)C1] | 481 |
| 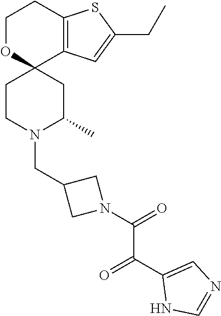 [SMILES: CCc1cc2c(s1)CCO[C@@]21CCN(CC2CN(C(=O)C(=O)c3cnc[nH]3)C2)[C@@H](C)C1] | 482 |
| 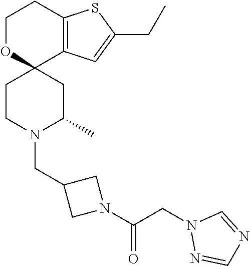 [SMILES: CCc1cc2c(s1)CCO[C@@]21CCN(CC2CN(C(=O)Cn3cncn3)C2)[C@@H](C)C1] | 483 |
| 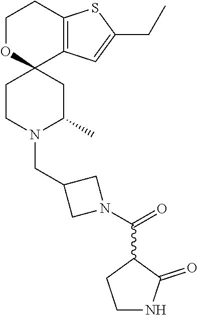 [SMILES: CCc1cc2c(s1)CCO[C@@]21CCN(CC2CN(C(=O)C3CCNC3=O)C2)[C@@H](C)C1] | 484 |
| 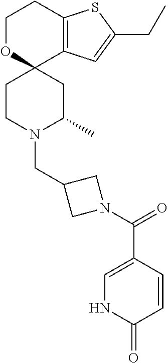 [SMILES: CCc1cc2c(s1)CCO[C@@]21CCN(CC2CN(C(=O)c3ccc(=O)[nH]c3)C2)[C@@H](C)C1] | 485 |
| 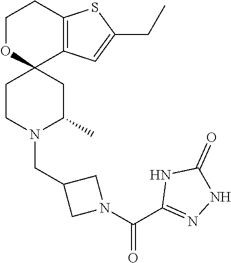 [SMILES: CCc1cc2c(s1)CCO[C@@]21CCN(CC2CN(C(=O)c3n[nH]c(=O)[nH]3)C2)[C@@H](C)C1] | 486 |
| 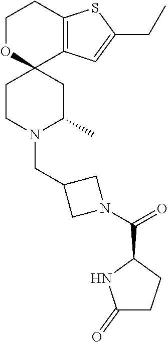 [SMILES: CCc1cc2c(s1)CCO[C@@]21CCN(CC2CN(C(=O)[C@H]3CCC(=O)N3)C2)[C@@H](C)C1] | 487 |
| 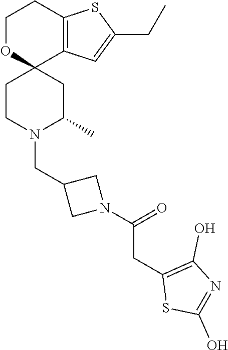 [SMILES: CCc1cc2c(s1)CCO[C@@]21CCN(CC2CN(C(=O)Cc3sc(O)nc3O)C2)[C@@H](C)C1] | 488 |
| 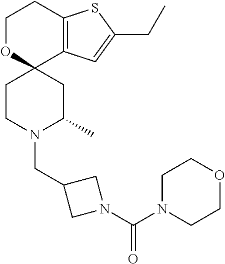 [SMILES: CCc1cc2c(s1)CCO[C@@]21CCN(CC2CN(C(=O)N3CCOCC3)C2)[C@@H](C)C1] | 489 |
| 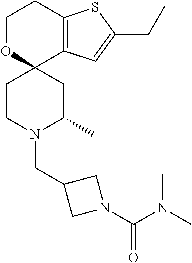 [SMILES: CCc1cc2c(s1)CCO[C@@]21CCN(CC2CN(C(=O)N(C)C)C2)[C@@H](C)C1] | 490 |
| 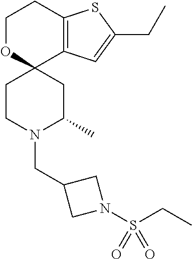 [SMILES: CCc1cc2c(s1)CCO[C@@]21CCN(CC2CN(S(=O)(=O)CC)C2)[C@@H](C)C1] | 491 |
| 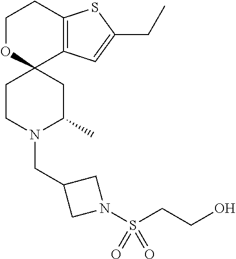 [SMILES: CCc1cc2c(s1)CCO[C@@]21CCN(CC2CN(S(=O)(=O)CCO)C2)[C@@H](C)C1] | 492 |
| 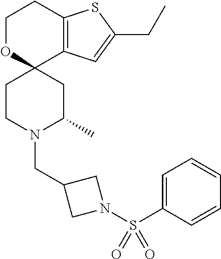 [SMILES: CCc1cc2c(s1)CCO[C@@]21CCN(CC2CN(S(=O)(=O)c3ccccc3)C2)[C@@H](C)C1] | 493 |
| 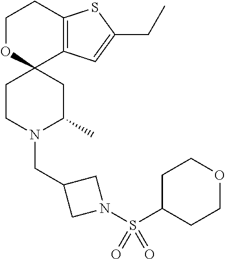 [SMILES: CCc1cc2c(s1)CCO[C@@]21CCN(CC2CN(S(=O)(=O)C3CCOCC3)C2)[C@@H](C)C1] | 494 |
| 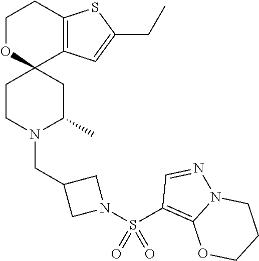 [SMILES: CCc1cc2c(s1)CCO[C@@]21CCN(CC2CN(S(=O)(=O)c3cnn4c3OCCC4)C2)[C@@H](C)C1] | 495 |
| 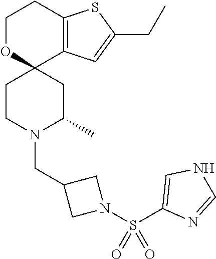 [SMILES: CCc1cc2c(s1)CCO[C@@]21CCN(CC2CN(S(=O)(=O)c3c[nH]cn3)C2)[C@@H](C)C1] | 496 |
| 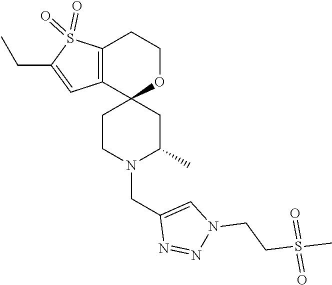 [SMILES: CCC1=CC2=C(CCO[C@@]23CCN(Cc2cn(CCS(C)(=O)=O)nn2)[C@@H](C)C3)S1(=O)=O] | 497 |
| 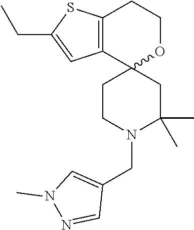 | 498 |
| 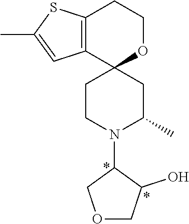 [SMILES: Cc1cc2c(s1)CCO[C@@]21CCN(C2COCC2O)[C@@H](C)C1] | 499 |
| 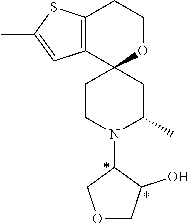 [SMILES: Cc1cc2c(s1)CCO[C@@]21CCN(C2COCC2O)[C@@H](C)C1] | 500 |
| 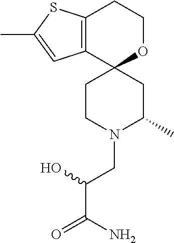 | 501 |
| 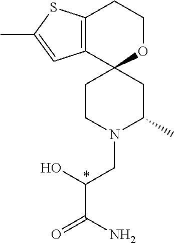 | 502 |
| 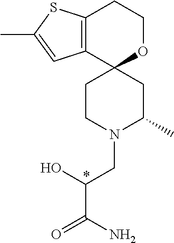 | 503 |
| 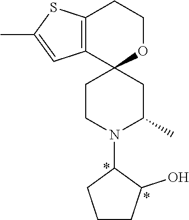 [SMILES: Cc1cc2c(s1)CCO[C@@]21CCN(C2CCCC2O)[C@@H](C)C1] | 504 |
| 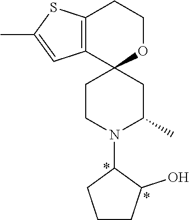 [SMILES: Cc1cc2c(s1)CCO[C@@]21CCN(C2CCCC2O)[C@@H](C)C1] | 505 |
| 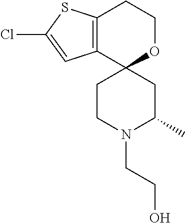 [SMILES: C[C@H]1C[C@@]2(CCN1CCO)OCCc1sc(Cl)cc12] | 506 |
| 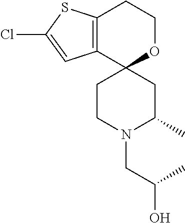 [SMILES: C[C@H](O)CN1CC[C@]2(C[C@@H]1C)OCCc1sc(Cl)cc12] | 507 |
| 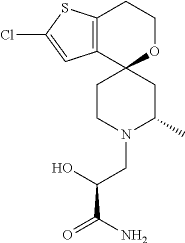 [SMILES: C[C@H]1C[C@@]2(CCN1C[C@H](O)C(N)=O)OCCc1sc(Cl)cc12] | 508 |
| 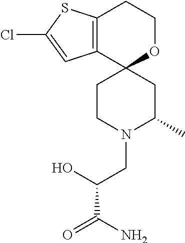 [SMILES: C[C@H]1C[C@@]2(CCN1C[C@@H](O)C(N)=O)OCCc1sc(Cl)cc12] | 509 |
| 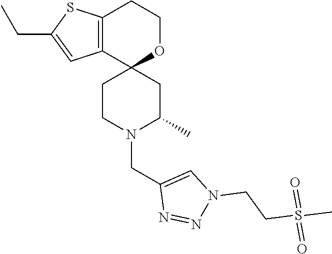 [SMILES: CCc1cc2c(s1)CCO[C@@]21CCN(Cc2cn(CCS(C)(=O)=O)nn2)[C@@H](C)C1] | 510 |
| 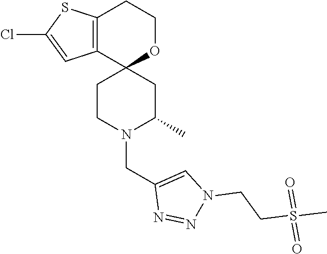 [SMILES: C[C@H]1C[C@@]2(CCN1Cc1cn(CCS(C)(=O)=O)nn1)OCCc1sc(Cl)cc12] | 511 |
| 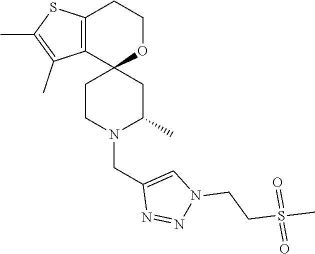 | 512 |
| 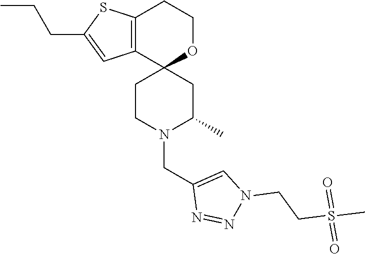 [SMILES: CCCc1cc2c(s1)CCO[C@@]21CCN(Cc2cn(CCS(C)(=O)=O)nn2)[C@@H](C)C1] | 513 |
| 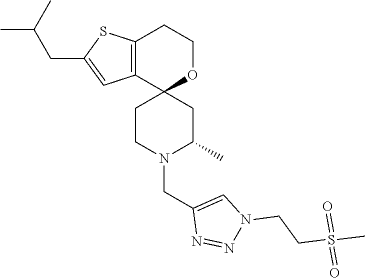 [SMILES: CC(C)Cc1cc2c(s1)CCO[C@@]21CCN(Cc2cn(CCS(C)(=O)=O)nn2)[C@@H](C)C1] | 514 |
| 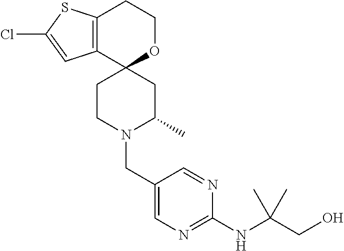 [SMILES: C[C@H]1C[C@@]2(CCN1Cc1cnc(NC(C)(C)CO)nc1)OCCc1sc(Cl)cc12] | 515 |
| 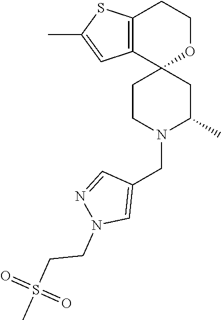 [SMILES: Cc1cc2c(s1)CCO[C@]21CCN(Cc2cnn(CCS(C)(=O)=O)c2)[C@@H](C)C1] | 516 |
| 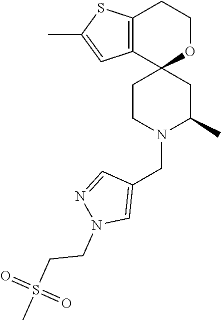 [SMILES: Cc1cc2c(s1)CCO[C@@]21CCN(Cc2cnn(CCS(C)(=O)=O)c2)[C@H](C)C1] | 517 |
| 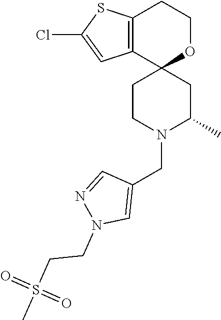 [SMILES: C[C@H]1C[C@@]2(CCN1Cc1cnn(CCS(C)(=O)=O)c1)OCCc1sc(Cl)cc12] | 518 |
| 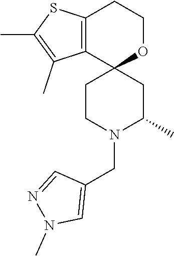 | 519 |
| 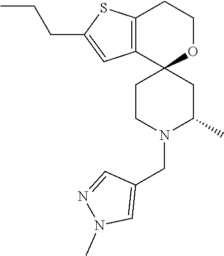 [SMILES: CCCc1cc2c(s1)CCO[C@@]21CCN(Cc2cnn(C)c2)[C@@H](C)C1] | 520 |
| 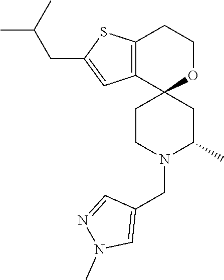 [SMILES: CC(C)Cc1cc2c(s1)CCO[C@@]21CCN(Cc2cnn(C)c2)[C@@H](C)C1] | 521 |
| 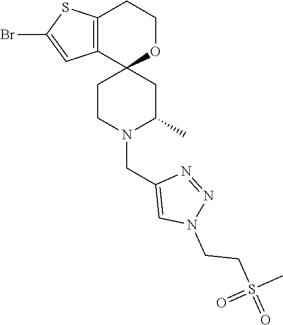 [SMILES: C[C@H]1C[C@@]2(CCN1Cc1cn(CCS(C)(=O)=O)nn1)OCCc1sc(Br)cc12] | 522 |
| 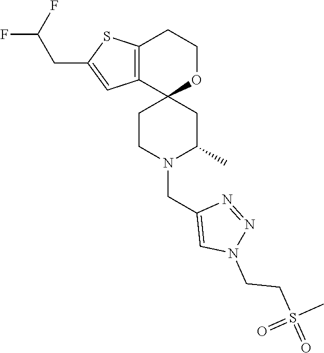 [SMILES: C[C@H]1C[C@@]2(CCN1Cc1cn(CCS(C)(=O)=O)nn1)OCCc1sc(CC(F)F)cc12] | 523 |
| 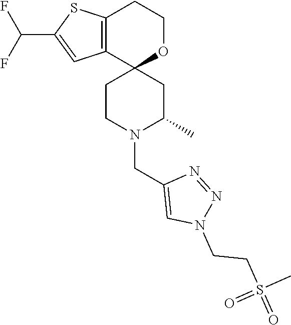 [SMILES: C[C@H]1C[C@@]2(CCN1Cc1cn(CCS(C)(=O)=O)nn1)OCCc1sc(C(F)F)cc12] | 524 |
| 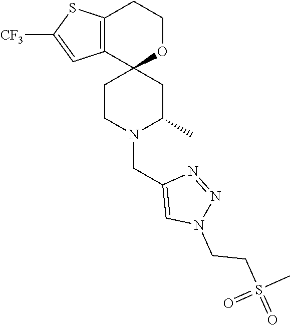 [SMILES: C[C@H]1C[C@@]2(CCN1Cc1cn(CCS(C)(=O)=O)nn1)OCCc1sc(C(F)(F)F)cc12] | 525 |
| 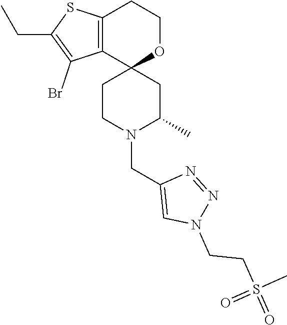 [SMILES: CCc1sc2c(c1Br)[C@]1(CCN(Cc3cn(CCS(C)(=O)=O)nn3)[C@@H](C)C1)OCC2] | 526 |
| 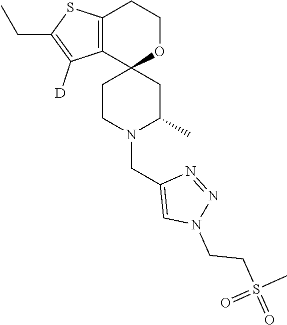 [SMILES: [2H]c1c(CC)sc2c1[C@]1(CCN(Cc3cn(CCS(C)(=O)=O)nn3)[C@@H](C)C1)OCC2] | 527 |
| 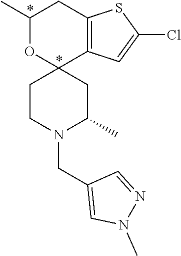 [SMILES: CC1Cc2sc(Cl)cc2C2(CCN(Cc3cnn(C)c3)[C@@H](C)C2)O1] | 528 |
| 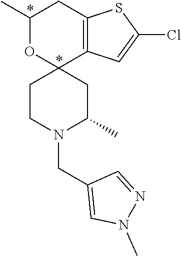 [SMILES: CC1Cc2sc(Cl)cc2C2(CCN(Cc3cnn(C)c3)[C@@H](C)C2)O1] | 529 |
| 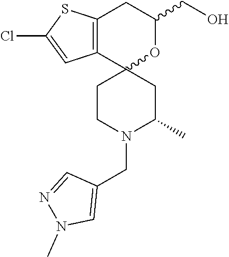 | 530 |
| 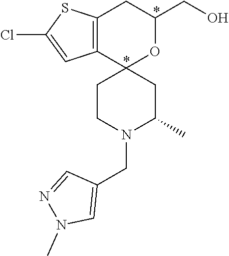 | 531 |
| 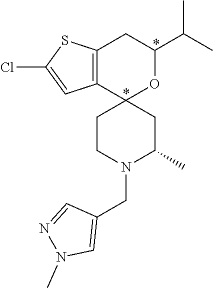 | 532 |
| 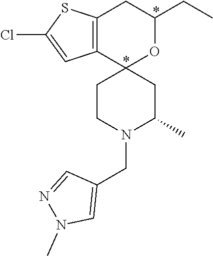 | 533 |
| 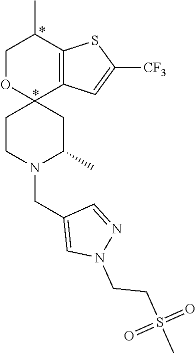 [SMILES: CC1COC2(CCN(Cc3cnn(CCS(C)(=O)=O)c3)[C@@H](C)C2)c2cc(C(F)(F)F)sc21] | 534 |
| 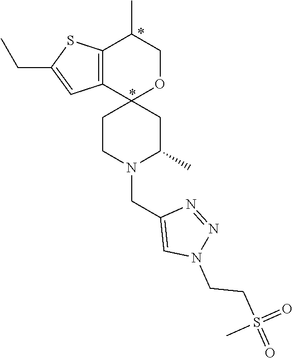 [SMILES: CCc1cc2c(s1)C(C)COC21CCN(Cc2cn(CCS(C)(=O)=O)nn2)[C@@H](C)C1] | 535 |
| 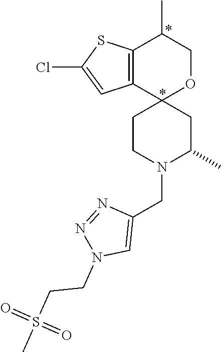 [SMILES: CC1COC2(CCN(Cc3cn(CCS(C)(=O)=O)nn3)[C@@H](C)C2)c2cc(Cl)sc21] | 536 |
| 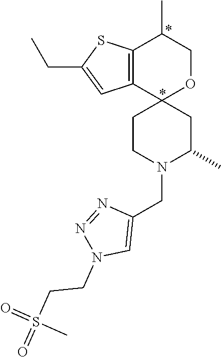 [SMILES: CCc1cc2c(s1)C(C)COC21CCN(Cc2cn(CCS(C)(=O)=O)nn2)[C@@H](C)C1] | 537 |
| 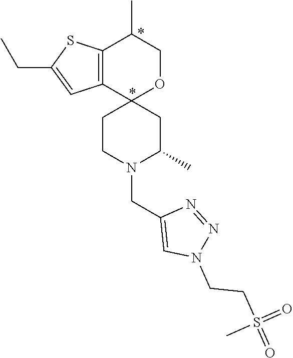 [SMILES: CCc1cc2c(s1)C(C)COC21CCN(Cc2cn(CCS(C)(=O)=O)nn2)[C@@H](C)C1] | 538 |
| 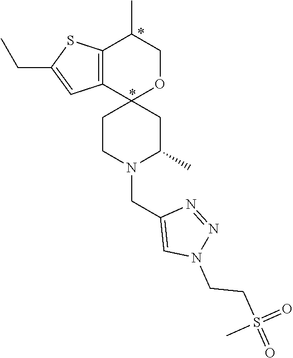 [SMILES: CCc1cc2c(s1)C(C)COC21CCN(Cc2cn(CCS(C)(=O)=O)nn2)[C@@H](C)C1] | 539 |
| 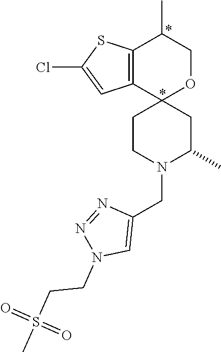 [SMILES: CC1COC2(CCN(Cc3cn(CCS(C)(=O)=O)nn3)[C@@H](C)C2)c2cc(Cl)sc21] | 540 |
| 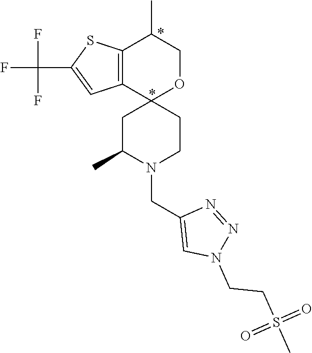 [SMILES: CC1COC2(CCN(Cc3cn(CCS(C)(=O)=O)nn3)[C@@H](C)C2)c2cc(C(F)(F)F)sc21] | 541 |
| 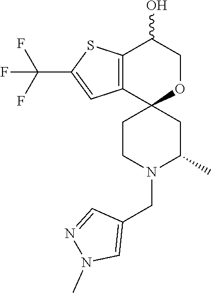 | 542 |
| 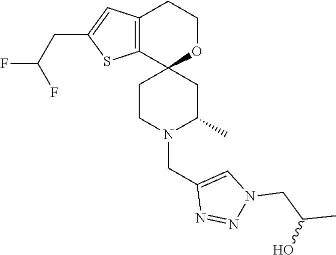 [SMILES: CC(O)Cn1cc(CN2CC[C@]3(C[C@@H]2C)OCCc2cc(CC(F)F)sc23)nn1] | 543 |
| 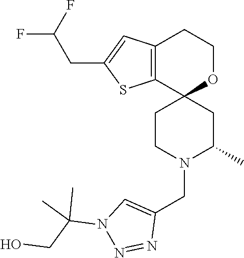 [SMILES: C[C@H]1C[C@@]2(CCN1Cc1cn(C(C)(C)CO)nn1)OCCc1cc(CC(F)F)sc12] | 544 |
| 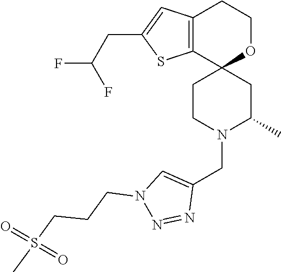 [SMILES: C[C@H]1C[C@@]2(CCN1Cc1cn(CCCS(C)(=O)=O)nn1)OCCc1cc(CC(F)F)sc12] | 545 |
| 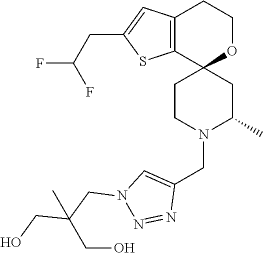 [SMILES: C[C@H]1C[C@@]2(CCN1Cc1cn(CC(C)(CO)CO)nn1)OCCc1cc(CC(F)F)sc12] | 546 |
| 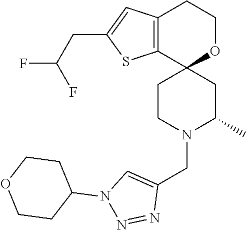 [SMILES: C[C@H]1C[C@@]2(CCN1Cc1cn(C3CCOCC3)nn1)OCCc1cc(CC(F)F)sc12] | 547 |
| 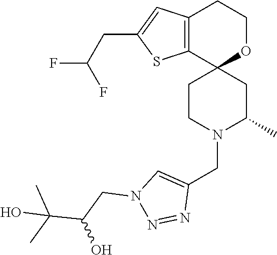 | 548 |
| 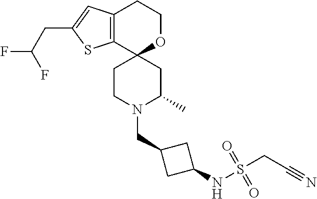 [SMILES: C[C@H]1C[C@@]2(CCN1C[C@H]1C[C@@H](NS(=O)(=O)CC#N)C1)OCCc1cc(CC(F)F)sc12] | 549 |
| 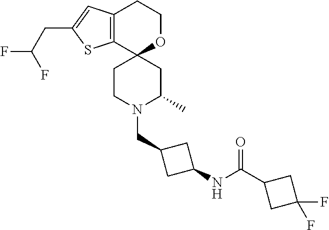 [SMILES: C[C@H]1C[C@@]2(CCN1C[C@H]1C[C@@H](NC(=O)C3CC(F)(F)C3)C1)OCCc1cc(CC(F)F)sc12] | 550 |
| 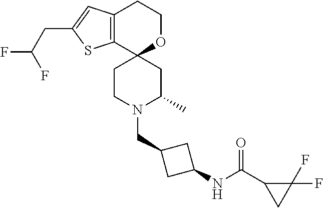 [SMILES: C[C@H]1C[C@@]2(CCN1C[C@H]1C[C@@H](NC(=O)C3CC3(F)F)C1)OCCc1cc(CC(F)F)sc12] | 551 |
| 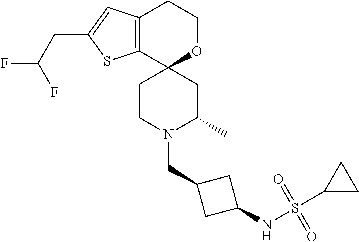 [SMILES: C[C@H]1C[C@@]2(CCN1C[C@H]1C[C@@H](NS(=O)(=O)C3CC3)C1)OCCc1cc(CC(F)F)sc12] | 552 |
| 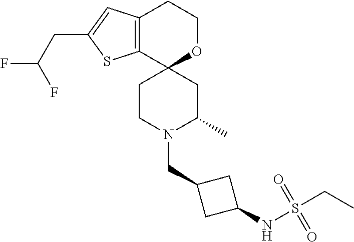 [SMILES: CCS(=O)(=O)N[C@H]1C[C@@H](CN2CC[C@]3(C[C@@H]2C)OCCc2cc(CC(F)F)sc23)C1] | 553 |
| 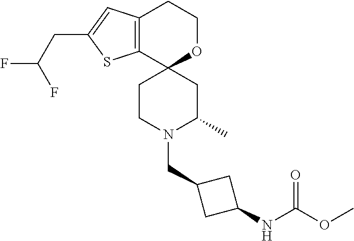 [SMILES: COC(=O)N[C@H]1C[C@@H](CN2CC[C@]3(C[C@@H]2C)OCCc2cc(CC(F)F)sc23)C1] | 554 |
| 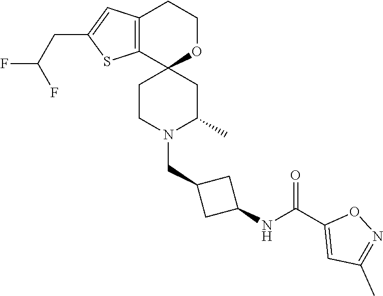 [SMILES: Cc1cc(C(=O)N[C@H]2C[C@@H](CN3CC[C@]4(C[C@@H]3C)OCCc3cc(CC(F)F)sc34)C2)on1] | 555 |
| 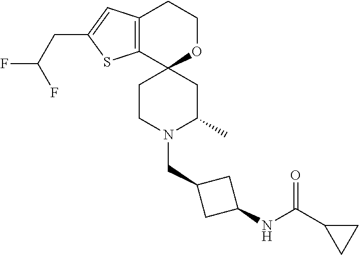 [SMILES: C[C@H]1C[C@@]2(CCN1C[C@H]1C[C@@H](NC(=O)C3CC3)C1)OCCc1cc(CC(F)F)sc12] | 556 |
| 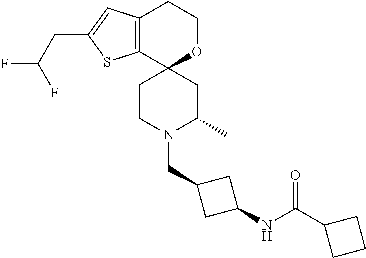 [SMILES: C[C@H]1C[C@@]2(CCN1C[C@H]1C[C@@H](NC(=O)C3CCC3)C1)OCCc1cc(CC(F)F)sc12] | 557 |
| 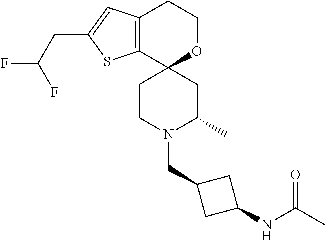 [SMILES: CC(=O)N[C@H]1C[C@@H](CN2CC[C@]3(C[C@@H]2C)OCCc2cc(CC(F)F)sc23)C1] | 558 |
| 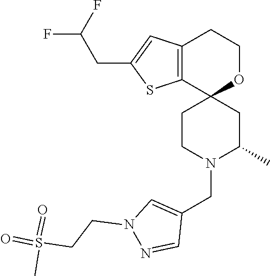 [SMILES: C[C@H]1C[C@@]2(CCN1Cc1cnn(CCS(C)(=O)=O)c1)OCCc1cc(CC(F)F)sc12] | 559 |
| 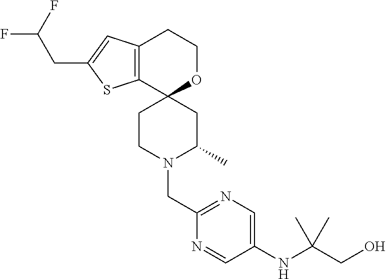 [SMILES: C[C@H]1C[C@@]2(CCN1Cc1ncc(NC(C)(C)CO)cn1)OCCc1cc(CC(F)F)sc12] | 560 |
| 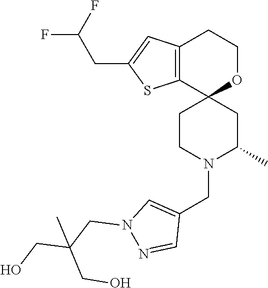 [SMILES: C[C@H]1C[C@@]2(CCN1Cc1cnn(CC(C)(CO)CO)c1)OCCc1cc(CC(F)F)sc12] | 561 |
| 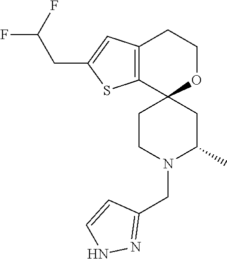 [SMILES: C[C@H]1C[C@@]2(CCN1Cc1cc[nH]n1)OCCc1cc(CC(F)F)sc12] | 562 |
| 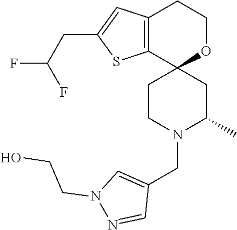 [SMILES: C[C@H]1C[C@@]2(CCN1Cc1cnn(CCO)c1)OCCc1cc(CC(F)F)sc12] | 563 |
| 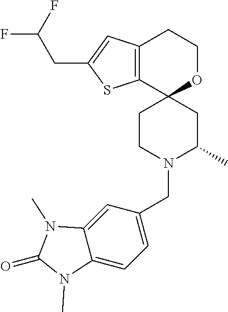 [SMILES: C[C@H]1C[C@@]2(CCN1Cc1ccc3c(c1)n(C)c(=O)n3C)OCCc1cc(CC(F)F)sc12] | 564 |
| 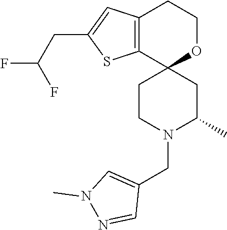 [SMILES: C[C@H]1C[C@@]2(CCN1Cc1cnn(C)c1)OCCc1cc(CC(F)F)sc12] | 565 |
| 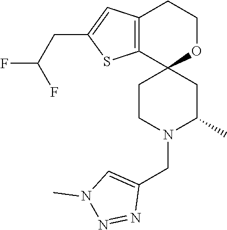 [SMILES: C[C@H]1C[C@@]2(CCN1Cc1cn(C)nn1)OCCc1cc(CC(F)F)sc12] | 566 |
| 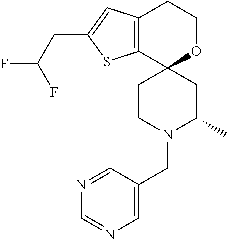 [SMILES: C[C@H]1C[C@@]2(CCN1Cc1cncnc1)OCCc1cc(CC(F)F)sc12] | 567 |
| 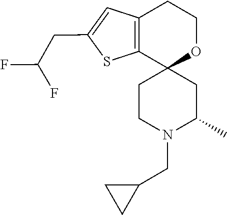 [SMILES: C[C@H]1C[C@@]2(CCN1CC1CC1)OCCc1cc(CC(F)F)sc12] | 568 |
| 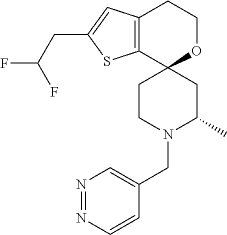 [SMILES: C[C@H]1C[C@@]2(CCN1Cc1ccnnc1)OCCc1cc(CC(F)F)sc12] | 569 |
| 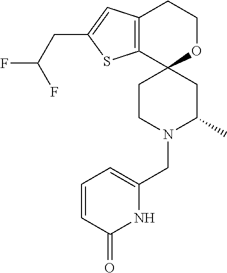 [SMILES: C[C@H]1C[C@@]2(CCN1Cc1cccc(=O)[nH]1)OCCc1cc(CC(F)F)sc12] | 570 |
| 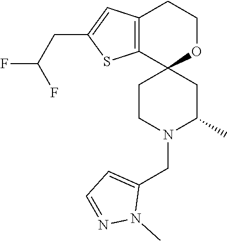 [SMILES: C[C@H]1C[C@@]2(CCN1Cc1ccnn1C)OCCc1cc(CC(F)F)sc12] | 571 |
| 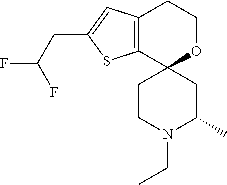 [SMILES: CCN1CC[C@]2(C[C@@H]1C)OCCc1cc(CC(F)F)sc12] | 572 |
| 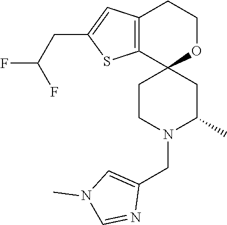 [SMILES: C[C@H]1C[C@@]2(CCN1Cc1cn(C)cn1)OCCc1cc(CC(F)F)sc12] | 573 |
| 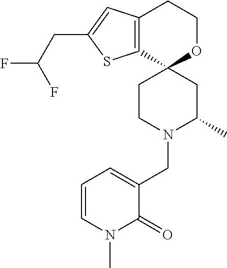 [SMILES: C[C@H]1C[C@@]2(CCN1Cc1cccn(C)c1=O)OCCc1cc(CC(F)F)sc12] | 574 |
| 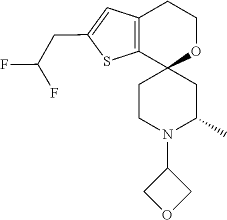 [SMILES: C[C@H]1C[C@@]2(CCN1C1COC1)OCCc1cc(CC(F)F)sc12] | 575 |
| 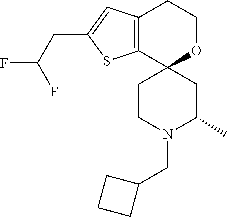 [SMILES: C[C@H]1C[C@@]2(CCN1CC1CCC1)OCCc1cc(CC(F)F)sc12] | 576 |
| 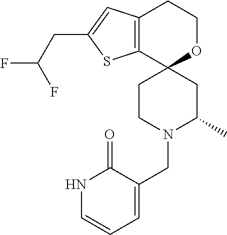 [SMILES: C[C@H]1C[C@@]2(CCN1Cc1ccc[nH]c1=O)OCCc1cc(CC(F)F)sc12] | 577 |
| 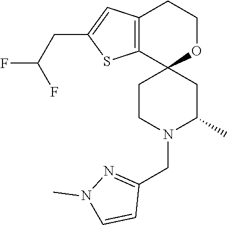 [SMILES: C[C@H]1C[C@@]2(CCN1Cc1ccn(C)n1)OCCc1cc(CC(F)F)sc12] | 578 |
| 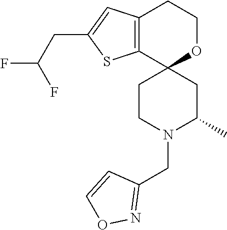 [SMILES: C[C@H]1C[C@@]2(CCN1Cc1ccon1)OCCc1cc(CC(F)F)sc12] | 579 |
| 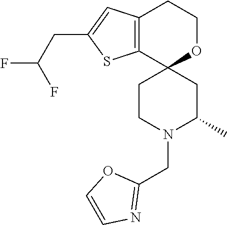 [SMILES: C[C@H]1C[C@@]2(CCN1Cc1ncco1)OCCc1cc(CC(F)F)sc12] | 580 |
| 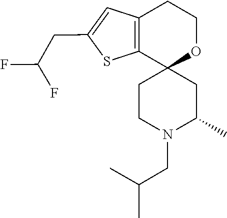 [SMILES: CC(C)CN1CC[C@]2(C[C@@H]1C)OCCc1cc(CC(F)F)sc12] | 581 |
| 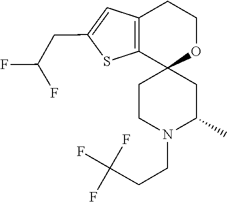 [SMILES: C[C@H]1C[C@@]2(CCN1CCC(F)(F)F)OCCc1cc(CC(F)F)sc12] | 582 |
| 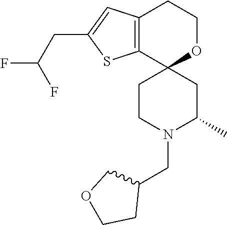 [SMILES: C[C@H]1C[C@@]2(CCN1CC1CCOC1)OCCc1cc(CC(F)F)sc12] | 583 |
| 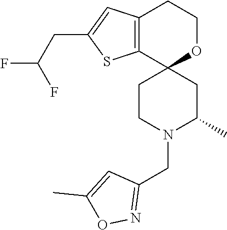 [SMILES: Cc1cc(CN2CC[C@]3(C[C@@H]2C)OCCc2cc(CC(F)F)sc23)no1] | 584 |
| 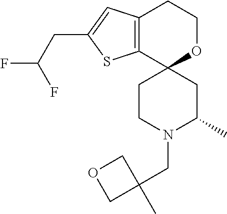 [SMILES: C[C@H]1C[C@@]2(CCN1CC1(C)COC1)OCCc1cc(CC(F)F)sc12] | 585 |
| 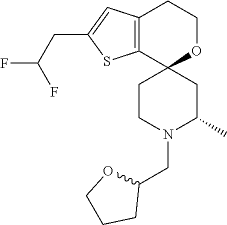 [SMILES: C[C@H]1C[C@@]2(CCN1CC1CCCO1)OCCc1cc(CC(F)F)sc12] | 586 |
| 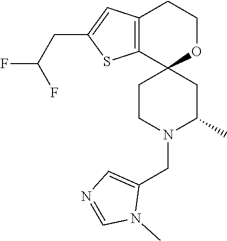 [SMILES: C[C@H]1C[C@@]2(CCN1Cc1cncn1C)OCCc1cc(CC(F)F)sc12] | 587 |
| 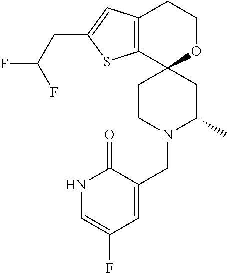 [SMILES: C[C@H]1C[C@@]2(CCN1Cc1cc(F)c[nH]c1=O)OCCc1cc(CC(F)F)sc12] | 588 |
| 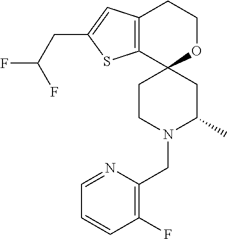 [SMILES: C[C@H]1C[C@@]2(CCN1Cc1ncccc1F)OCCc1cc(CC(F)F)sc12] | 589 |
| 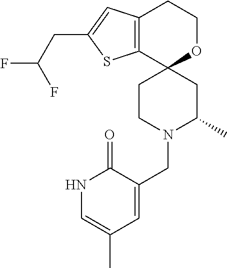 [SMILES: Cc1c[nH]c(=O)c(CN2CC[C@]3(C[C@@H]2C)OCCc2cc(CC(F)F)sc23)c1] | 590 |
| 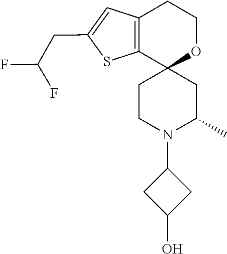 [SMILES: C[C@H]1C[C@@]2(CCN1C1CC(O)C1)OCCc1cc(CC(F)F)sc12] | 591 |
| 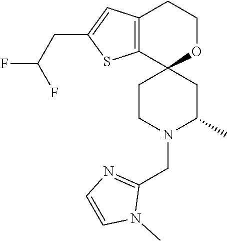 [SMILES: C[C@H]1C[C@@]2(CCN1Cc1nccn1C)OCCc1cc(CC(F)F)sc12] | 592 |
| 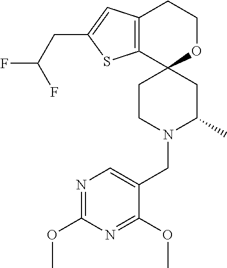 [SMILES: COc1ncc(CN2CC[C@]3(C[C@@H]2C)OCCc2cc(CC(F)F)sc23)c(OC)n1] | 593 |
| 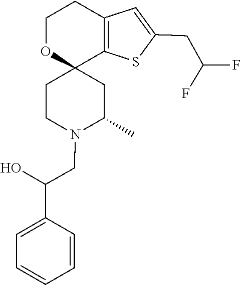 [SMILES: C[C@H]1C[C@@]2(CCN1CC(O)c1ccccc1)OCCc1cc(CC(F)F)sc12] | 594 |
| 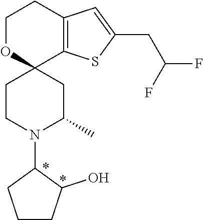 [SMILES: C[C@H]1C[C@@]2(CCN1C1CCCC1O)OCCc1cc(CC(F)F)sc12] | 595 |
| 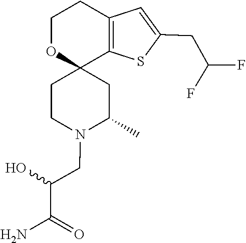 [SMILES: C[C@H]1C[C@@]2(CCN1CC(O)C(N)=O)OCCc1cc(CC(F)F)sc12] | 596 |
| 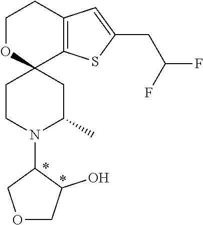 [SMILES: C[C@H]1C[C@@]2(CCN1C1COCC1O)OCCc1cc(CC(F)F)sc12] | 597 |
| 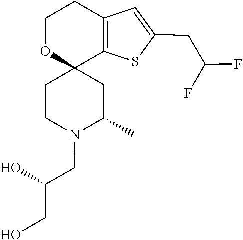 [SMILES: C[C@H]1C[C@@]2(CCN1C[C@@H](O)CO)OCCc1cc(CC(F)F)sc12] | 598 |
| 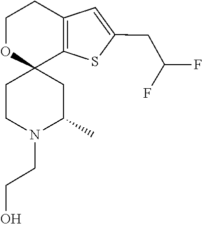 | 599 |
| 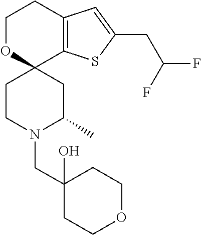 [SMILES: C[C@H]1C[C@@]2(CCN1CC1(O)CCOCC1)OCCc1cc(CC(F)F)sc12] | 600 |
| 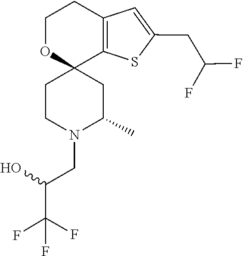 [SMILES: C[C@H]1C[C@@]2(CCN1CC(O)C(F)(F)F)OCCc1cc(CC(F)F)sc12] | 601 |
| 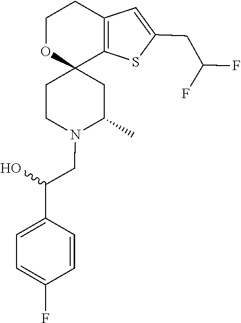 [SMILES: C[C@H]1C[C@@]2(CCN1CC(O)c1ccc(F)cc1)OCCc1cc(CC(F)F)sc12] | 602 |
| 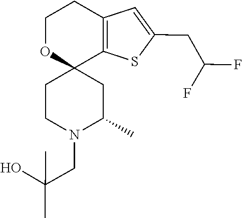 [SMILES: C[C@H]1C[C@@]2(CCN1CC(C)(C)O)OCCc1cc(CC(F)F)sc12] | 603 |
| 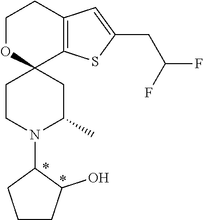 [SMILES: C[C@H]1C[C@@]2(CCN1C1CCCC1O)OCCc1cc(CC(F)F)sc12] | 604 |
| 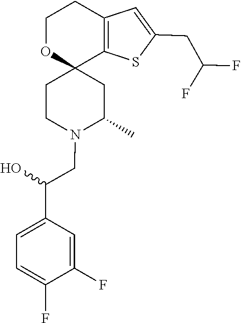 [SMILES: C[C@H]1C[C@@]2(CCN1CC(O)c1ccc(F)c(F)c1)OCCc1cc(CC(F)F)sc12] | 605 |
| 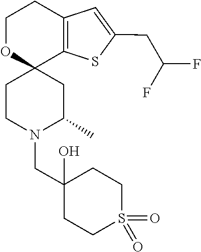 [SMILES: C[C@H]1C[C@@]2(CCN1CC1(O)CCS(=O)(=O)CC1)OCCc1cc(CC(F)F)sc12] | 606 |
| 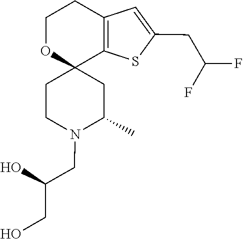 [SMILES: C[C@H]1C[C@@]2(CCN1C[C@H](O)CO)OCCc1cc(CC(F)F)sc12] | 607 |
| 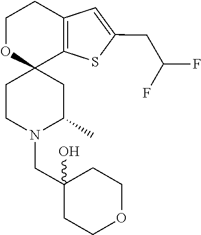 [SMILES: C[C@H]1C[C@@]2(CCN1CC1(O)CCOCC1)OCCc1cc(CC(F)F)sc12] | 608 |
| 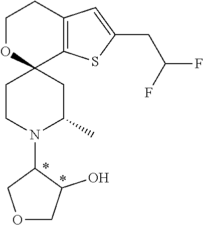 [SMILES: C[C@H]1C[C@@]2(CCN1C1COCC1O)OCCc1cc(CC(F)F)sc12] | 609 |
| 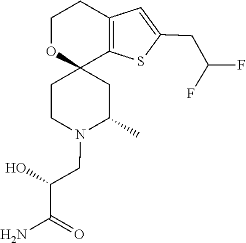 [SMILES: C[C@H]1C[C@@]2(CCN1C[C@@H](O)C(N)=O)OCCc1cc(CC(F)F)sc12] | 610 |
| 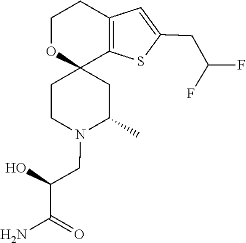 [SMILES: C[C@H]1C[C@@]2(CCN1C[C@H](O)C(N)=O)OCCc1cc(CC(F)F)sc12] | 611 |
| 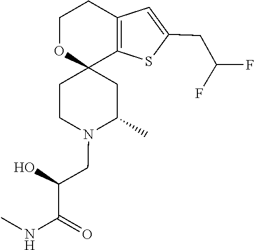 [SMILES: CNC(=O)[C@@H](O)CN1CC[C@]2(C[C@@H]1C)OCCc1cc(CC(F)F)sc12] | 612 |
| 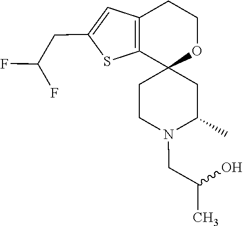 [SMILES: CC(O)CN1CC[C@]2(C[C@@H]1C)OCCc1cc(CC(F)F)sc12] | 613 |
| 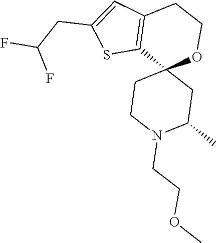 [SMILES: COCCN1CC[C@]2(C[C@@H]1C)OCCc1cc(CC(F)F)sc12] | 614 |
| 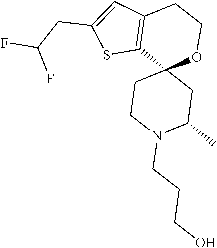 [SMILES: C[C@H]1C[C@@]2(CCN1CCCO)OCCc1cc(CC(F)F)sc12] | 615 |
| 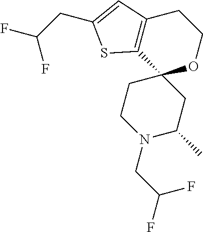 [SMILES: C[C@H]1C[C@@]2(CCN1CC(F)F)OCCc1cc(CC(F)F)sc12] | 616 |
| 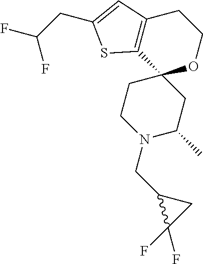 [SMILES: C[C@H]1C[C@@]2(CCN1CC1CC1(F)F)OCCc1cc(CC(F)F)sc12] | 617 |
| 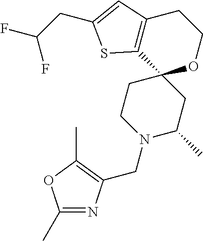 [SMILES: Cc1nc(CN2CC[C@]3(C[C@@H]2C)OCCc2cc(CC(F)F)sc23)c(C)o1] | 618 |
| 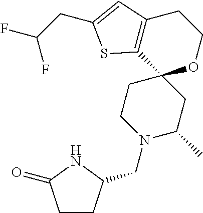 | 619 |
| 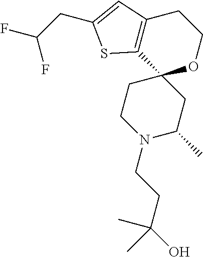 [SMILES: C[C@H]1C[C@@]2(CCN1CCC(C)(C)O)OCCc1cc(CC(F)F)sc12] | 620 |
| 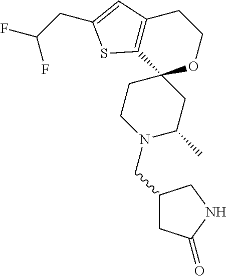 [SMILES: C[C@H]1C[C@@]2(CCN1CC1CNC(=O)C1)OCCc1cc(CC(F)F)sc12] | 621 |
| 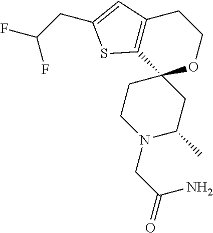 [SMILES: C[C@H]1C[C@@]2(CCN1CC(N)=O)OCCc1cc(CC(F)F)sc12] | 622 |
| 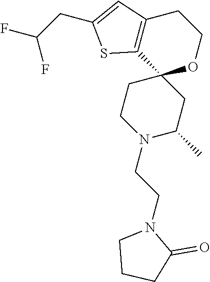 [SMILES: C[C@H]1C[C@@]2(CCN1CCN1CCCC1=O)OCCc1cc(CC(F)F)sc12] | 623 |
| 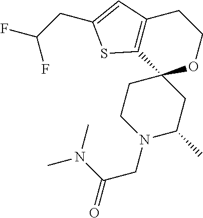 [SMILES: C[C@H]1C[C@@]2(CCN1CC(=O)N(C)C)OCCc1cc(CC(F)F)sc12] | 624 |
| 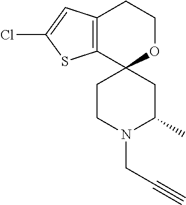 [SMILES: C#CCN1CC[C@]2(C[C@@H]1C)OCCc1cc(Cl)sc12] | 625 |
| 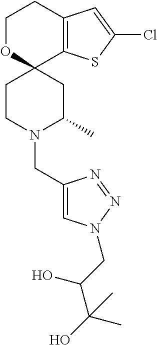 [SMILES: C[C@H]1C[C@@]2(CCN1Cc1cn(CC(O)C(C)(C)O)nn1)OCCc1cc(Cl)sc12] | 626 |
| 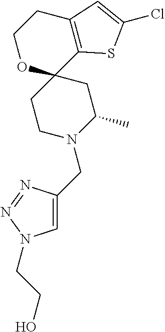 [SMILES: C[C@H]1C[C@@]2(CCN1Cc1cn(CCO)nn1)OCCc1cc(Cl)sc12] | 627 |
| 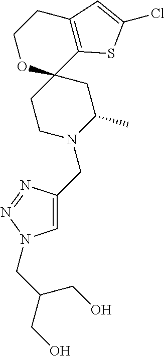 [SMILES: C[C@H]1C[C@@]2(CCN1Cc1cn(CC(CO)CO)nn1)OCCc1cc(Cl)sc12] | 628 |
| 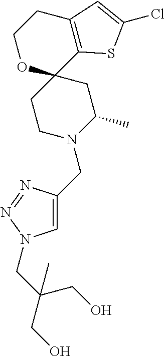 [SMILES: C[C@H]1C[C@@]2(CCN1Cc1cn(CC(C)(CO)CO)nn1)OCCc1cc(Cl)sc12] | 629 |
| 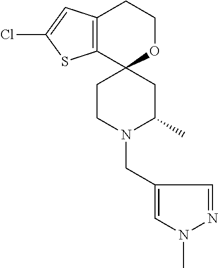 [SMILES: C[C@H]1C[C@@]2(CCN1Cc1cnn(C)c1)OCCc1cc(Cl)sc12] | 630 |
| 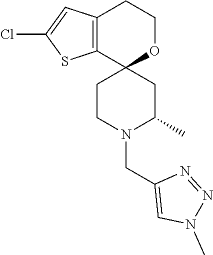 [SMILES: C[C@H]1C[C@@]2(CCN1Cc1cn(C)nn1)OCCc1cc(Cl)sc12] | 631 |
| 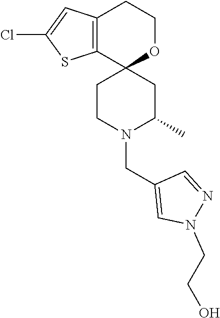 [SMILES: C[C@H]1C[C@@]2(CCN1Cc1cnn(CCO)c1)OCCc1cc(Cl)sc12] | 632 |
| 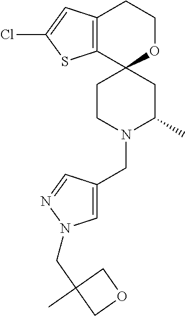 [SMILES: C[C@H]1C[C@@]2(CCN1Cc1cnn(CC3(C)COC3)c1)OCCc1cc(Cl)sc12] | 633 |
| 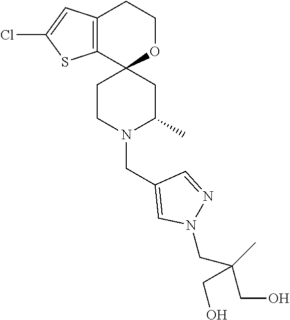 [SMILES: C[C@H]1C[C@@]2(CCN1Cc1cnn(CC(C)(CO)CO)c1)OCCc1cc(Cl)sc12] | 634 |
| 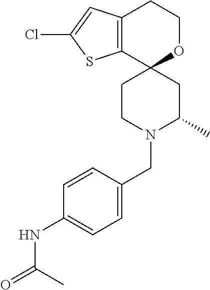 [SMILES: CC(=O)Nc1ccc(CN2CC[C@]3(C[C@@H]2C)OCCc2cc(Cl)sc23)cc1] | 635 |
| 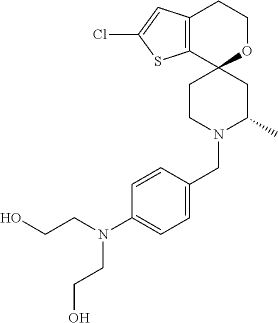 [SMILES: C[C@H]1C[C@@]2(CCN1Cc1ccc(N(CCO)CCO)cc1)OCCc1cc(Cl)sc12] | 636 |
| 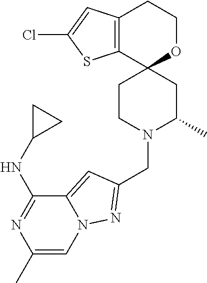 [SMILES: Cc1cn2nc(CN3CC[C@]4(C[C@@H]3C)OCCc3cc(Cl)sc34)cc2c(NC2CC2)n1] | 637 |
| 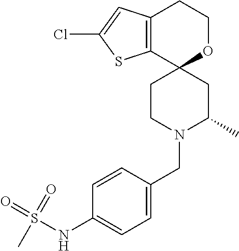 [SMILES: C[C@H]1C[C@@]2(CCN1Cc1ccc(NS(C)(=O)=O)cc1)OCCc1cc(Cl)sc12] | 638 |
| 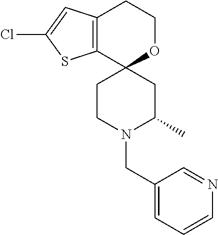 [SMILES: C[C@H]1C[C@@]2(CCN1Cc1cccnc1)OCCc1cc(Cl)sc12] | 639 |
| 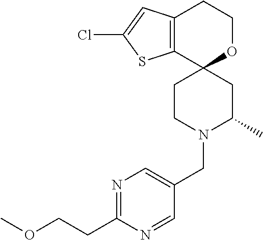 | 640 |
| 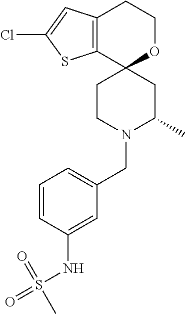 [SMILES: C[C@H]1C[C@@]2(CCN1Cc1cccc(NS(C)(=O)=O)c1)OCCc1cc(Cl)sc12] | 641 |
| 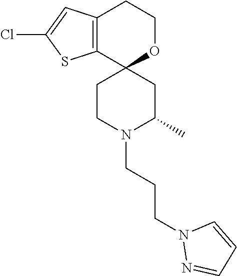 [SMILES: C[C@H]1C[C@@]2(CCN1CCCn1cccn1)OCCc1cc(Cl)sc12] | 642 |
| 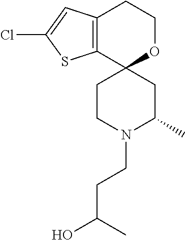 [SMILES: CC(O)CCN1CC[C@]2(C[C@@H]1C)OCCc1cc(Cl)sc12] | 643 |
| 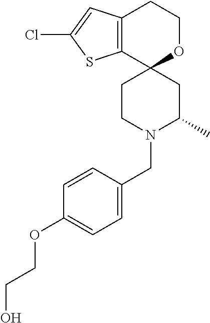 [SMILES: C[C@H]1C[C@@]2(CCN1Cc1ccc(OCCO)cc1)OCCc1cc(Cl)sc12] | 644 |
| 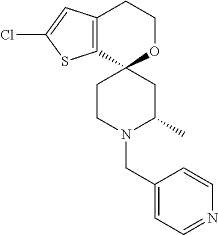 [SMILES: C[C@H]1C[C@@]2(CCN1Cc1ccncc1)OCCc1cc(Cl)sc12] | 645 |
| 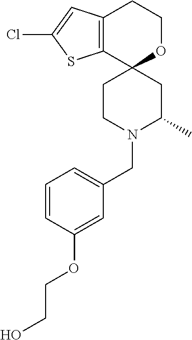 [SMILES: C[C@H]1C[C@@]2(CCN1Cc1cccc(OCCO)c1)OCCc1cc(Cl)sc12] | 646 |
| 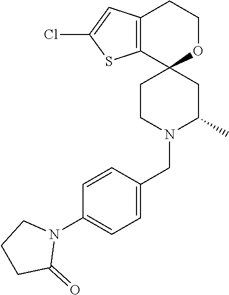 [SMILES: C[C@H]1C[C@@]2(CCN1Cc1ccc(N3CCCC3=O)cc1)OCCc1cc(Cl)sc12] | 647 |
| 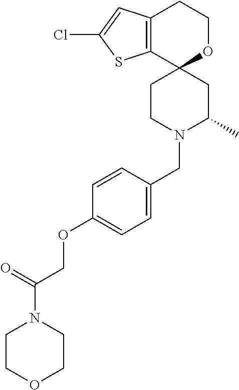 [SMILES: C[C@H]1C[C@@]2(CCN1Cc1ccc(OCC(=O)N3CCOCC3)cc1)OCCc1cc(Cl)sc12] | 648 |
| 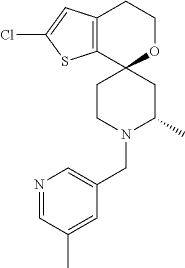 [SMILES: Cc1cncc(CN2CC[C@]3(C[C@@H]2C)OCCc2cc(Cl)sc23)c1] | 649 |
| 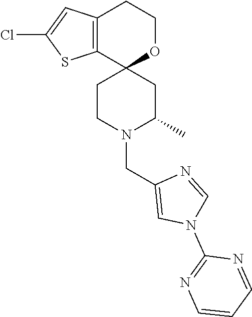 [SMILES: C[C@H]1C[C@@]2(CCN1Cc1cn(-c3ncccn3)cn1)OCCc1cc(Cl)sc12] | 650 |
| 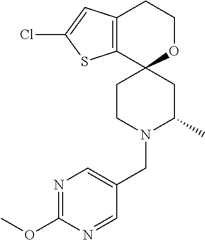 | 651 |
| 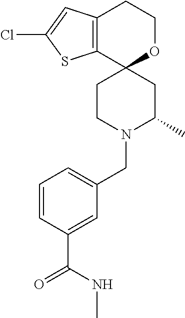 [SMILES: CNC(=O)c1cccc(CN2CC[C@]3(C[C@@H]2C)OCCc2cc(Cl)sc23)c1] | 652 |
| 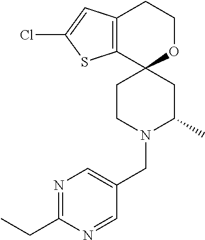 [SMILES: CCc1ncc(CN2CC[C@]3(C[C@@H]2C)OCCc2cc(Cl)sc23)cn1] | 653 |
| 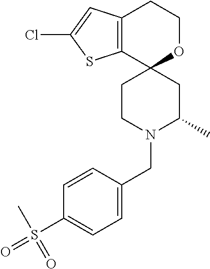 [SMILES: C[C@H]1C[C@@]2(CCN1Cc1ccc(S(C)(=O)=O)cc1)OCCc1cc(Cl)sc12] | 654 |
| 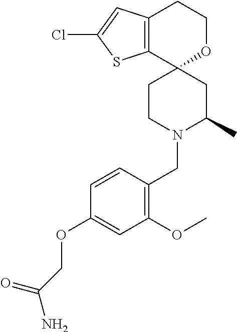 [SMILES: COc1cc(OCC(N)=O)ccc1CN1CC[C@@]2(C[C@H]1C)OCCc1cc(Cl)sc12] | 655 |
| 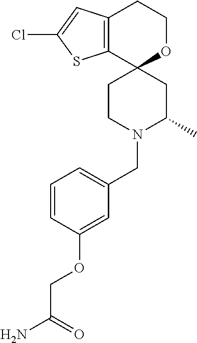 [SMILES: C[C@H]1C[C@@]2(CCN1Cc1cccc(OCC(N)=O)c1)OCCc1cc(Cl)sc12] | 656 |
| 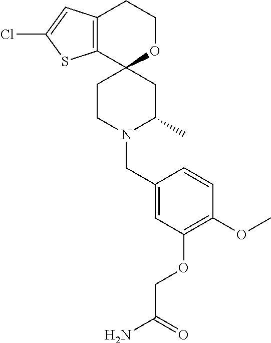 [SMILES: COc1ccc(CN2CC[C@]3(C[C@@H]2C)OCCc2cc(Cl)sc23)cc1OCC(N)=O] | 657 |
| 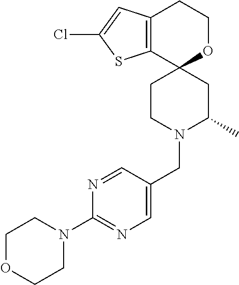 [SMILES: C[C@H]1C[C@@]2(CCN1Cc1cnc(N3CCOCC3)nc1)OCCc1cc(Cl)sc12] | 658 |
| 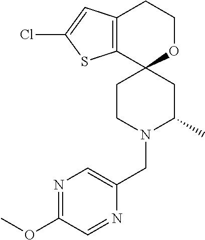 | 659 |
| 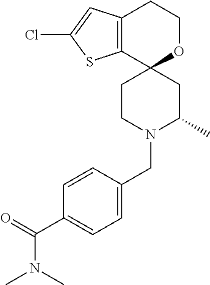 | 660 |
| 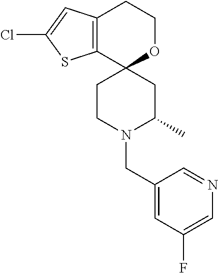 [SMILES: C[C@H]1C[C@@]2(CCN1Cc1cncc(F)c1)OCCc1cc(Cl)sc12] | 661 |
| 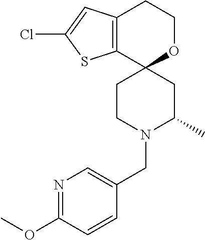 | 662 |
| 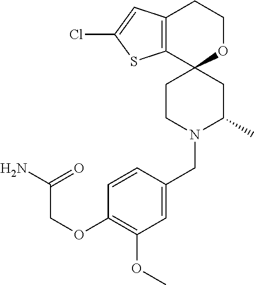 [SMILES: COc1cc(CN2CC[C@]3(C[C@@H]2C)OCCc2cc(Cl)sc23)ccc1OCC(N)=O] | 663 |
| 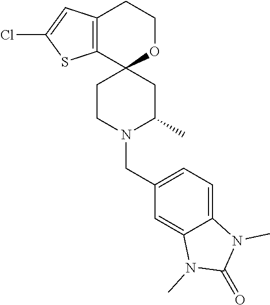 [SMILES: C[C@H]1C[C@@]2(CCN1Cc1ccc3c(c1)n(C)c(=O)n3C)OCCc1cc(Cl)sc12] | 664 |
| 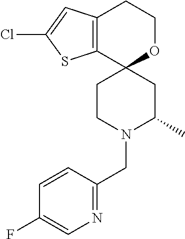 [SMILES: C[C@H]1C[C@@]2(CCN1Cc1ccc(F)cn1)OCCc1cc(Cl)sc12] | 665 |
| 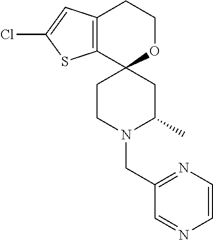 [SMILES: C[C@H]1C[C@@]2(CCN1Cc1cnccn1)OCCc1cc(Cl)sc12] | 666 |
| 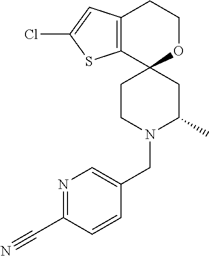 [SMILES: C[C@H]1C[C@@]2(CCN1Cc1ccc(C#N)nc1)OCCc1cc(Cl)sc12] | 667 |
| 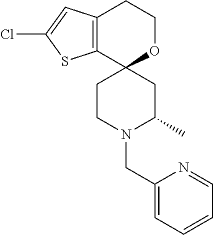 [SMILES: C[C@H]1C[C@@]2(CCN1Cc1ccccn1)OCCc1cc(Cl)sc12] | 668 |
| 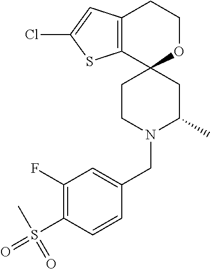 [SMILES: C[C@H]1C[C@@]2(CCN1Cc1ccc(S(C)(=O)=O)c(F)c1)OCCc1cc(Cl)sc12] | 669 |
| 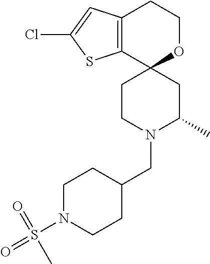 [SMILES: C[C@H]1C[C@@]2(CCN1CC1CCN(S(C)(=O)=O)CC1)OCCc1cc(Cl)sc12] | 670 |
| 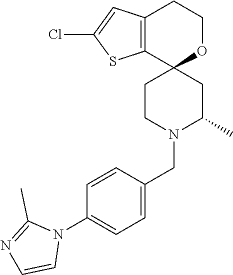 [SMILES: Cc1nccn1-c1ccc(CN2CC[C@]3(C[C@@H]2C)OCCc2cc(Cl)sc23)cc1] | 671 |
| 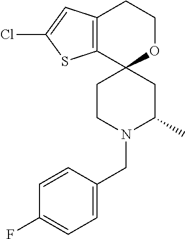 [SMILES: C[C@H]1C[C@@]2(CCN1Cc1ccc(F)cc1)OCCc1cc(Cl)sc12] | 672 |
| 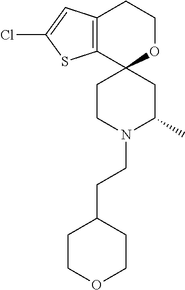 [SMILES: C[C@H]1C[C@@]2(CCN1CCC1CCOCC1)OCCc1cc(Cl)sc12] | 673 |
| 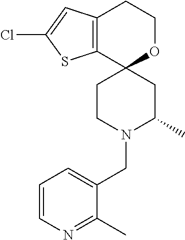 [SMILES: Cc1ncccc1CN1CC[C@]2(C[C@@H]1C)OCCc1cc(Cl)sc12] | 674 |
| 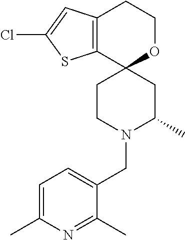 | 675 |
| 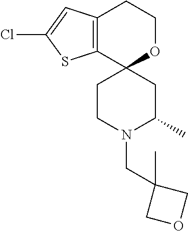 [SMILES: C[C@H]1C[C@@]2(CCN1CC1(C)COC1)OCCc1cc(Cl)sc12] | 676 |
| 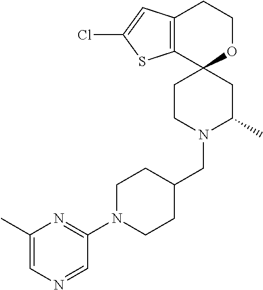 | 677 |
| 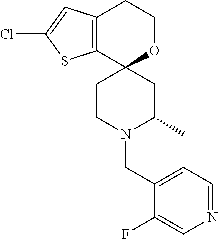 [SMILES: C[C@H]1C[C@@]2(CCN1Cc1ccncc1F)OCCc1cc(Cl)sc12] | 678 |
| 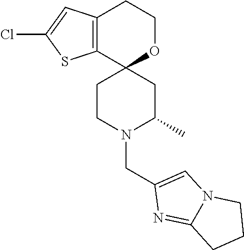 [SMILES: C[C@H]1C[C@@]2(CCN1Cc1cn3c(n1)CCC3)OCCc1cc(Cl)sc12] | 679 |
| 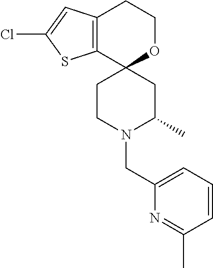 [SMILES: Cc1cccc(CN2CC[C@]3(C[C@@H]2C)OCCc2cc(Cl)sc23)n1] | 680 |
| 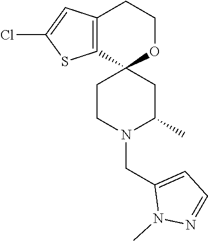 [SMILES: C[C@H]1C[C@@]2(CCN1Cc1ccnn1C)OCCc1cc(Cl)sc12] | 681 |
| 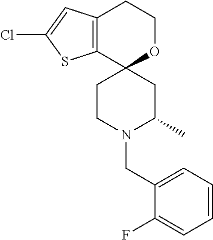 [SMILES: C[C@H]1C[C@@]2(CCN1Cc1ccccc1F)OCCc1cc(Cl)sc12] | 682 |
| 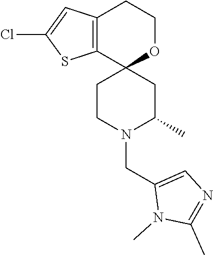 [SMILES: Cc1ncc(CN2CC[C@]3(C[C@@H]2C)OCCc2cc(Cl)sc23)n1C] | 683 |
| 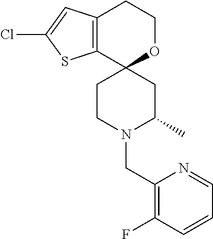 [SMILES: C[C@H]1C[C@@]2(CCN1Cc1ncccc1F)OCCc1cc(Cl)sc12] | 684 |
| 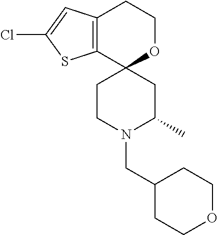 [SMILES: C[C@H]1C[C@@]2(CCN1CC1CCOCC1)OCCc1cc(Cl)sc12] | 685 |
| 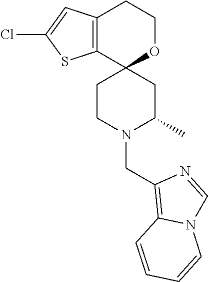 [SMILES: C[C@H]1C[C@@]2(CCN1Cc1ncn3ccccc13)OCCc1cc(Cl)sc12] | 686 |
| 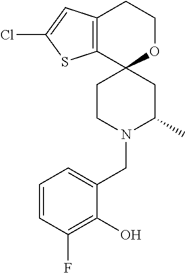 [SMILES: C[C@H]1C[C@@]2(CCN1Cc1cccc(F)c1O)OCCc1cc(Cl)sc12] | 687 |
| 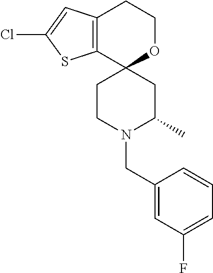 [SMILES: C[C@H]1C[C@@]2(CCN1Cc1cccc(F)c1)OCCc1cc(Cl)sc12] | 688 |
| 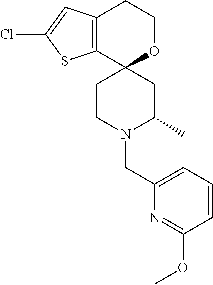 [SMILES: COc1cccc(CN2CC[C@]3(C[C@@H]2C)OCCc2cc(Cl)sc23)n1] | 689 |
| 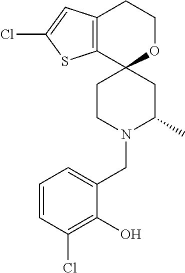 [SMILES: C[C@H]1C[C@@]2(CCN1Cc1cccc(Cl)c1O)OCCc1cc(Cl)sc12] | 690 |
| 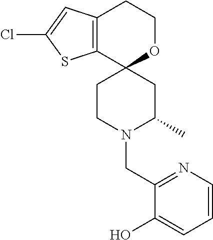 [SMILES: C[C@H]1C[C@@]2(CCN1Cc1ncccc1O)OCCc1cc(Cl)sc12] | 691 |
| 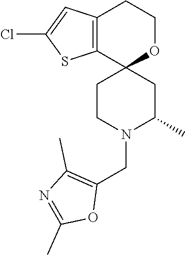 [SMILES: Cc1nc(C)c(CN2CC[C@]3(C[C@@H]2C)OCCc2cc(Cl)sc23)o1] | 692 |
| 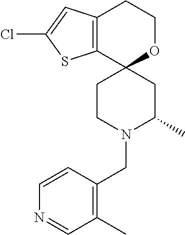 [SMILES: Cc1cnccc1CN1CC[C@]2(C[C@@H]1C)OCCc1cc(Cl)sc12] | 693 |
| 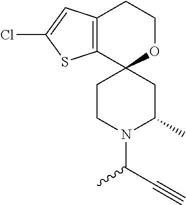 [SMILES: C#CC(C)N1CC[C@]2(C[C@@H]1C)OCCc1cc(Cl)sc12] | 694 |
| 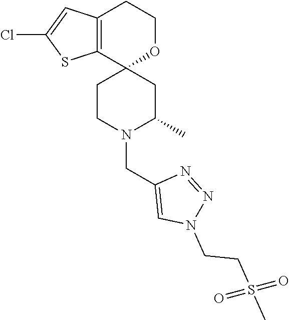 [SMILES: C[C@H]1C[C@]2(CCN1Cc1cn(CCS(C)(=O)=O)nn1)OCCc1cc(Cl)sc12] | 695 |
| 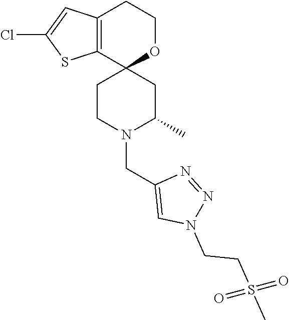 [SMILES: C[C@H]1C[C@@]2(CCN1Cc1cn(CCS(C)(=O)=O)nn1)OCCc1cc(Cl)sc12] | 696 |
| 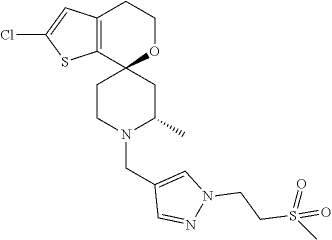 [SMILES: C[C@H]1C[C@@]2(CCN1Cc1cnn(CCS(C)(=O)=O)c1)OCCc1cc(Cl)sc12] | 697 |
| 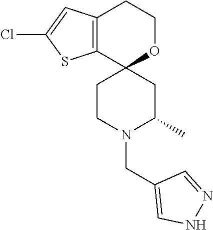 [SMILES: C[C@H]1C[C@@]2(CCN1Cc1cn[nH]c1)OCCc1cc(Cl)sc12] | 698 |
| 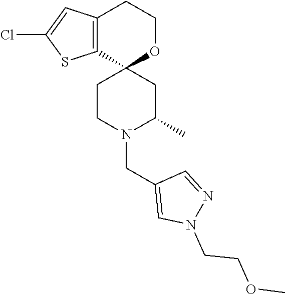 [SMILES: COCCn1cc(CN2CC[C@]3(C[C@@H]2C)OCCc2cc(Cl)sc23)cn1] | 699 |
| 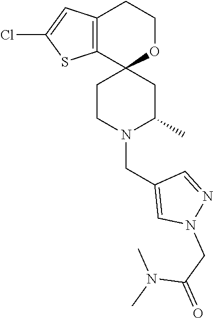 [SMILES: C[C@H]1C[C@@]2(CCN1Cc1cnn(CC(=O)N(C)C)c1)OCCc1cc(Cl)sc12] | 700 |
| 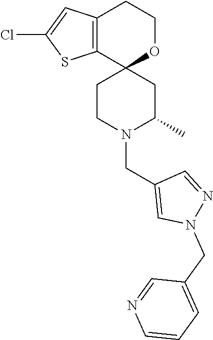 [SMILES: C[C@H]1C[C@@]2(CCN1Cc1cnn(Cc3cccnc3)c1)OCCc1cc(Cl)sc12] | 701 |
| 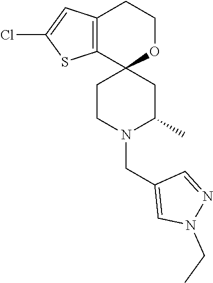 [SMILES: CCn1cc(CN2CC[C@]3(C[C@@H]2C)OCCc2cc(Cl)sc23)cn1] | 702 |
| 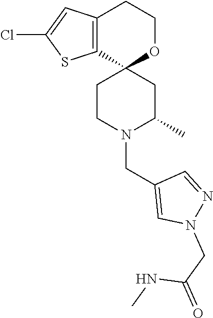 [SMILES: CNC(=O)Cn1cc(CN2CC[C@]3(C[C@@H]2C)OCCc2cc(Cl)sc23)cn1] | 703 |
| 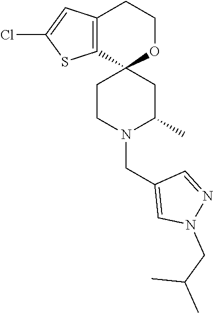 [SMILES: CC(C)Cn1cc(CN2CC[C@]3(C[C@@H]2C)OCCc2cc(Cl)sc23)cn1] | 704 |
| 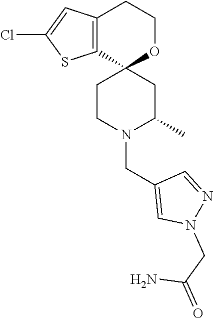 [SMILES: C[C@H]1C[C@@]2(CCN1Cc1cnn(CC(N)=O)c1)OCCc1cc(Cl)sc12] | 705 |
| 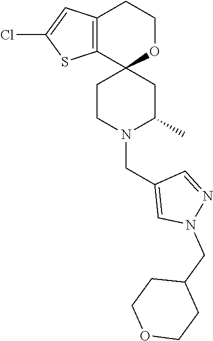 [SMILES: C[C@H]1C[C@@]2(CCN1Cc1cnn(CC3CCOCC3)c1)OCCc1cc(Cl)sc12] | 706 |
| 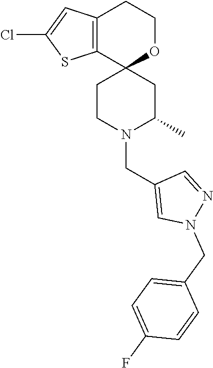 [SMILES: C[C@H]1C[C@@]2(CCN1Cc1cnn(Cc3ccc(F)cc3)c1)OCCc1cc(Cl)sc12] | 707 |
| 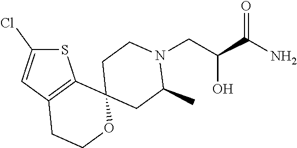 [SMILES: C[C@H]1C[C@@]2(CCN1C[C@H](O)C(N)=O)OCCc1cc(Cl)sc12] | 708 |
| 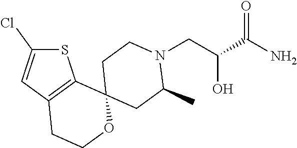 [SMILES: C[C@H]1C[C@@]2(CCN1C[C@@H](O)C(N)=O)OCCc1cc(Cl)sc12] | 709 |
| 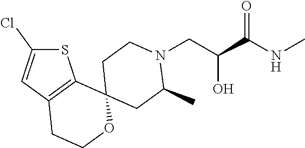 [SMILES: CNC(=O)[C@@H](O)CN1CC[C@]2(C[C@@H]1C)OCCc1cc(Cl)sc12] | 710 |
| 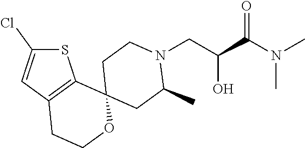 [SMILES: C[C@H]1C[C@@]2(CCN1C[C@H](O)C(=O)N(C)C)OCCc1cc(Cl)sc12] | 711 |
| 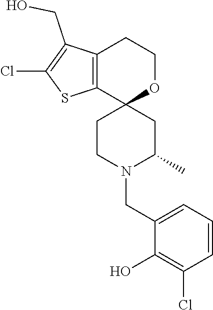 [SMILES: C[C@H]1C[C@@]2(CCN1Cc1cccc(Cl)c1O)OCCc1c2sc(Cl)c1CO] | 712 |
| 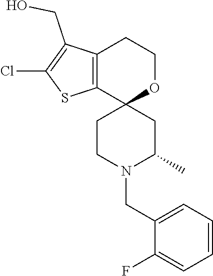 [SMILES: C[C@H]1C[C@@]2(CCN1Cc1ccccc1F)OCCc1c2sc(Cl)c1CO] | 713 |
| 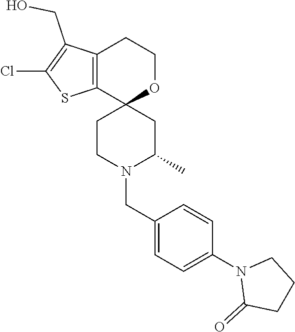 [SMILES: C[C@H]1C[C@@]2(CCN1Cc1ccc(N3CCCC3=O)cc1)OCCc1c2sc(Cl)c1CO] | 714 |
| 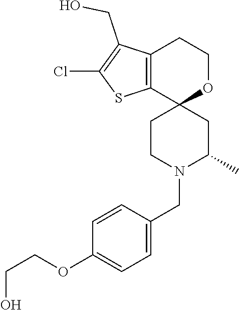 [SMILES: C[C@H]1C[C@@]2(CCN1Cc1ccc(OCCO)cc1)OCCc1c2sc(Cl)c1CO] | 715 |
| 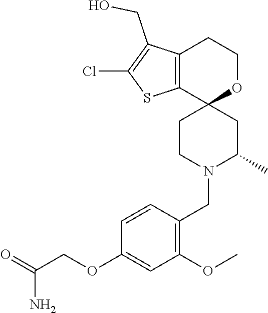 [SMILES: COc1cc(OCC(N)=O)ccc1CN1CC[C@]2(C[C@@H]1C)OCCc1c2sc(Cl)c1CO] | 716 |
| 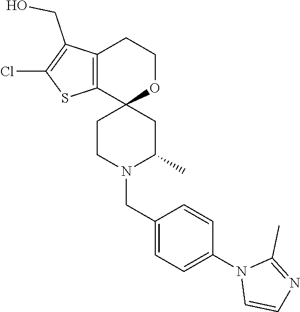 [SMILES: Cc1nccn1-c1ccc(CN2CC[C@]3(C[C@@H]2C)OCCc2c3sc(Cl)c2CO)cc1] | 717 |
| 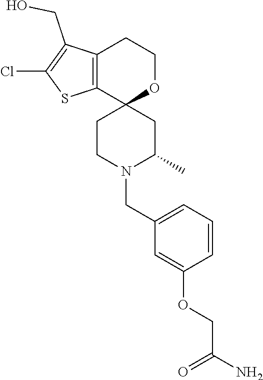 [SMILES: C[C@H]1C[C@@]2(CCN1Cc1cccc(OCC(N)=O)c1)OCCc1c2sc(Cl)c1CO] | 718 |
| 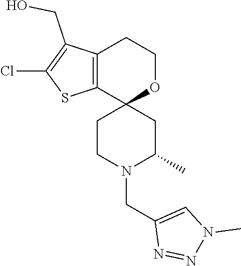 [SMILES: C[C@H]1C[C@@]2(CCN1Cc1cn(C)nn1)OCCc1c2sc(Cl)c1CO] | 719 |
| 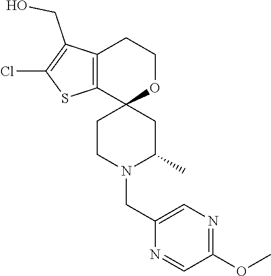 [SMILES: COc1cnc(CN2CC[C@]3(C[C@@H]2C)OCCc2c3sc(Cl)c2CO)cn1] | 720 |
| 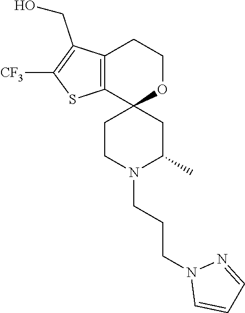 [SMILES: C[C@H]1C[C@@]2(CCN1CCCn1cccn1)OCCc1c2sc(C(F)(F)F)c1CO] | 721 |
| 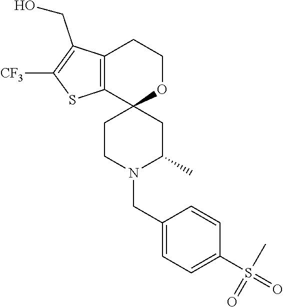 [SMILES: C[C@H]1C[C@@]2(CCN1Cc1ccc(S(C)(=O)=O)cc1)OCCc1c2sc(C(F)(F)F)c1CO] | 722 |
| 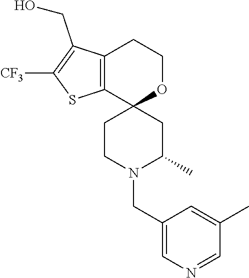 [SMILES: Cc1cncc(CN2CC[C@]3(C[C@@H]2C)OCCc2c3sc(C(F)(F)F)c2CO)c1] | 723 |
| 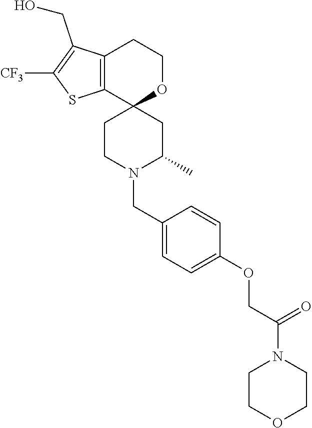 [SMILES: C[C@H]1C[C@@]2(CCN1Cc1ccc(OCC(=O)N3CCOCC3)cc1)OCCc1c2sc(C(F)(F)F)c1CO] | 724 |
| 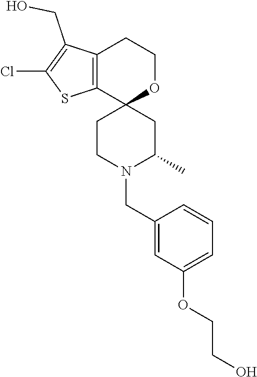 [SMILES: C[C@H]1C[C@@]2(CCN1Cc1cccc(OCCO)c1)OCCc1c2sc(Cl)c1CO] | 725 |
| 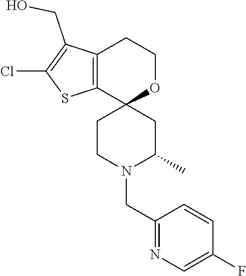 [SMILES: C[C@H]1C[C@@]2(CCN1Cc1ccc(F)cn1)OCCc1c2sc(Cl)c1CO] | 726 |
| 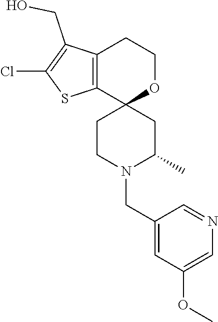 [SMILES: COc1cncc(CN2CC[C@]3(C[C@@H]2C)OCCc2c3sc(Cl)c2CO)c1] | 727 |
| 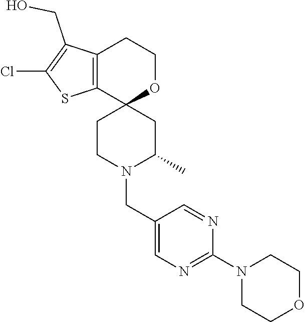 [SMILES: C[C@H]1C[C@@]2(CCN1Cc1cnc(N3CCOCC3)nc1)OCCc1c2sc(Cl)c1CO] | 728 |
| 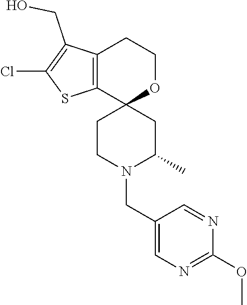 [SMILES: COc1ncc(CN2CC[C@]3(C[C@@H]2C)OCCc2c3sc(Cl)c2CO)cn1] | 729 |
| 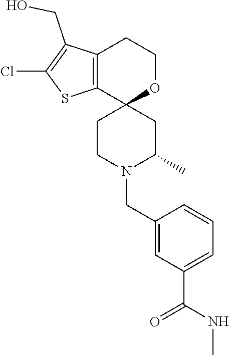 [SMILES: CNC(=O)c1cccc(CN2CC[C@]3(C[C@@H]2C)OCCc2c3sc(Cl)c2CO)c1] | 730 |
| 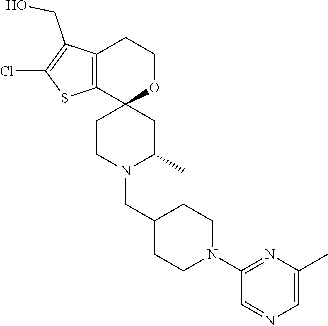 [SMILES: Cc1cncc(N2CCC(CN3CC[C@]4(C[C@@H]3C)OCCc3c4sc(Cl)c3CO)CC2)n1] | 731 |
| 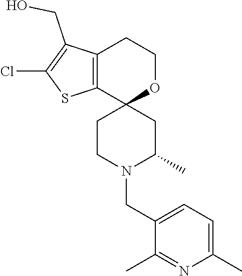 [SMILES: Cc1ccc(CN2CC[C@]3(C[C@@H]2C)OCCc2c3sc(Cl)c2CO)c(C)n1] | 732 |
| 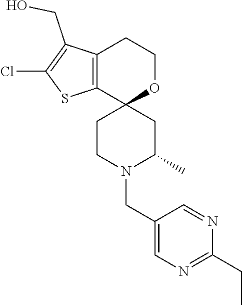 [SMILES: CCc1ncc(CN2CC[C@]3(C[C@@H]2C)OCCc2c3sc(Cl)c2CO)cn1] | 733 |
| 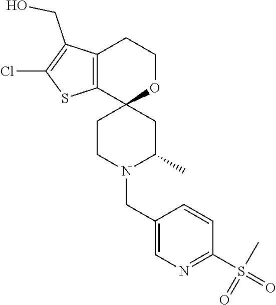 [SMILES: C[C@H]1C[C@@]2(CCN1Cc1ccc(S(C)(=O)=O)nc1)OCCc1c2sc(Cl)c1CO] | 734 |
| 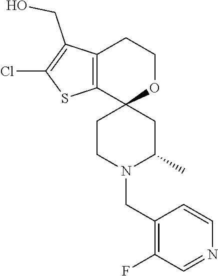 [SMILES: C[C@H]1C[C@@]2(CCN1Cc1ccncc1F)OCCc1c2sc(Cl)c1CO] | 735 |
| 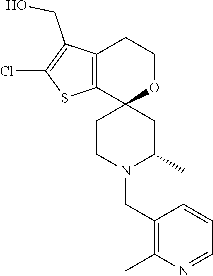 [SMILES: Cc1ncccc1CN1CC[C@]2(C[C@@H]1C)OCCc1c2sc(Cl)c1CO] | 736 |
| 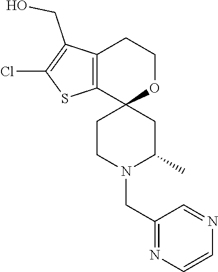 [SMILES: C[C@H]1C[C@@]2(CCN1Cc1cnccn1)OCCc1c2sc(Cl)c1CO] | 737 |
| 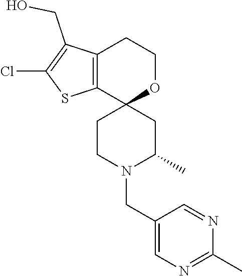 [SMILES: Cc1ncc(CN2CC[C@]3(C[C@@H]2C)OCCc2c3sc(Cl)c2CO)cn1] | 738 |
| 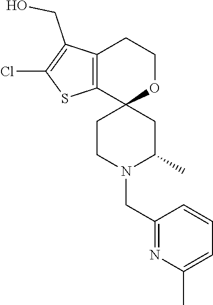 [SMILES: Cc1cccc(CN2CC[C@]3(C[C@@H]2C)OCCc2c3sc(Cl)c2CO)n1] | 739 |
| 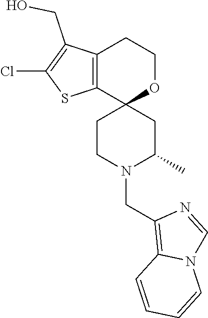 [SMILES: C[C@H]1C[C@@]2(CCN1Cc1ncn3ccccc13)OCCc1c2sc(Cl)c1CO] | 740 |
| 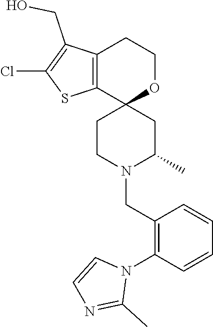 [SMILES: Cc1nccn1-c1ccccc1CN1CC[C@]2(C[C@@H]1C)OCCc1c2sc(Cl)c1CO] | 741 |
| 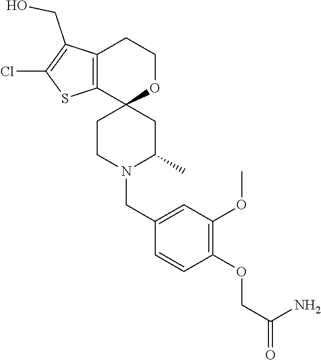 [SMILES: COc1cc(CN2CC[C@]3(C[C@@H]2C)OCCc2c3sc(Cl)c2CO)ccc1OCC(N)=O] | 742 |
| 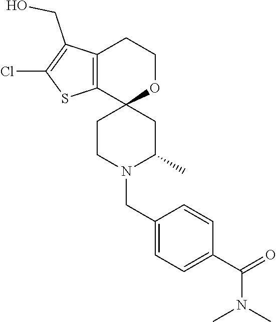 [SMILES: C[C@H]1C[C@@]2(CCN1Cc1ccc(C(=O)N(C)C)cc1)OCCc1c2sc(Cl)c1CO] | 743 |
| 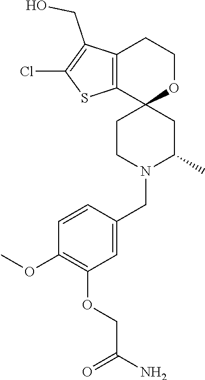 | 744 |
| 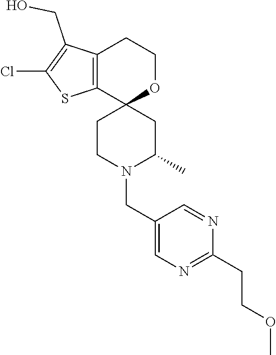 [SMILES: COCCc1ncc(CN2CC[C@]3(C[C@@H]2C)OCCc2c3sc(Cl)c2CO)cn1] | 745 |
| 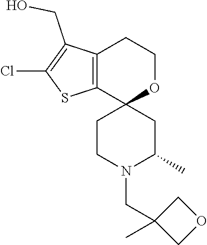 [SMILES: C[C@H]1C[C@@]2(CCN1CC1(C)COC1)OCCc1c2sc(Cl)c1CO] | 746 |
| 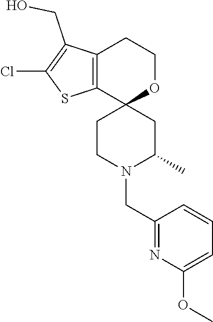 [SMILES: COc1cccc(CN2CC[C@]3(C[C@@H]2C)OCCc2c3sc(Cl)c2CO)n1] | 747 |
| 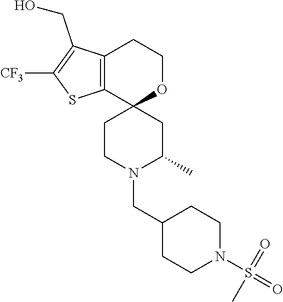 [SMILES: C[C@H]1C[C@@]2(CCN1CC1CCN(S(C)(=O)=O)CC1)OCCc1c2sc(C(F)(F)F)c1CO] | 748 |
| 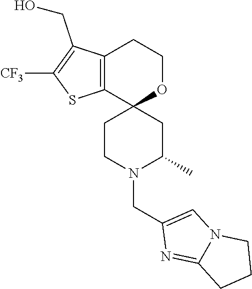 [SMILES: C[C@H]1C[C@@]2(CCN1Cc1cn3c(n1)CCC3)OCCc1c2sc(C(F)(F)F)c1CO] | 749 |
| 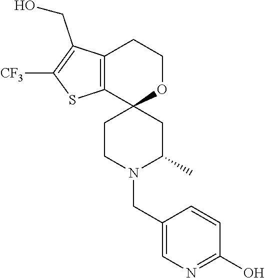 [SMILES: C[C@H]1C[C@@]2(CCN1Cc1ccc(O)nc1)OCCc1c2sc(C(F)(F)F)c1CO] | 750 |
| 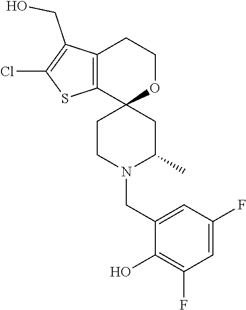 [SMILES: C[C@H]1C[C@@]2(CCN1Cc1cc(F)cc(F)c1O)OCCc1c2sc(Cl)c1CO] | 751 |
| 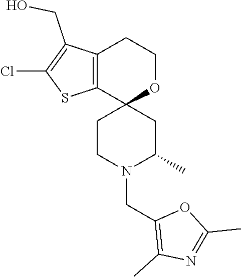 [SMILES: Cc1nc(C)c(CN2CC[C@]3(C[C@@H]2C)OCCc2c3sc(Cl)c2CO)o1] | 752 |
| 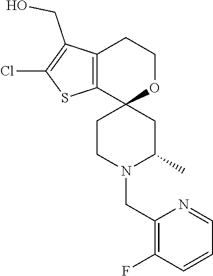 [SMILES: C[C@H]1C[C@@]2(CCN1Cc1ncccc1F)OCCc1c2sc(Cl)c1CO] | 753 |
| 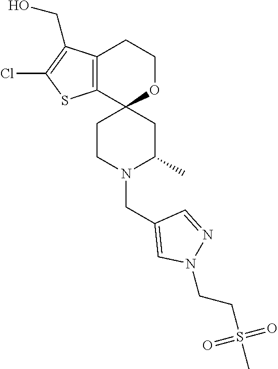 [SMILES: C[C@H]1C[C@@]2(CCN1Cc1cnn(CCS(C)(=O)=O)c1)OCCc1c2sc(Cl)c1CO] | 754 |
| 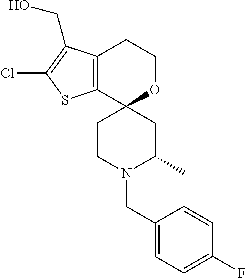 [SMILES: C[C@H]1C[C@@]2(CCN1Cc1ccc(F)cc1)OCCc1c2sc(Cl)c1CO] | 755 |
| 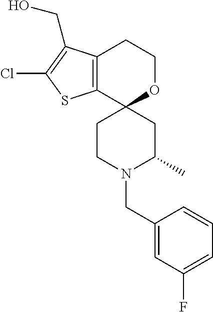 [SMILES: C[C@H]1C[C@@]2(CCN1Cc1cccc(F)c1)OCCc1c2sc(Cl)c1CO] | 756 |
| 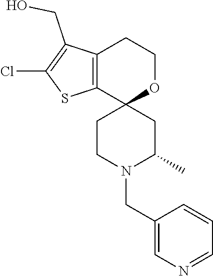 [SMILES: C[C@H]1C[C@@]2(CCN1Cc1cccnc1)OCCc1c2sc(Cl)c1CO] | 757 |
| 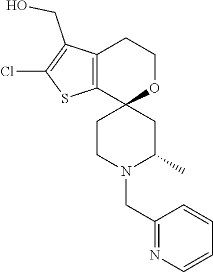 [SMILES: C[C@H]1C[C@@]2(CCN1Cc1ccccn1)OCCc1c2sc(Cl)c1CO] | 758 |
| 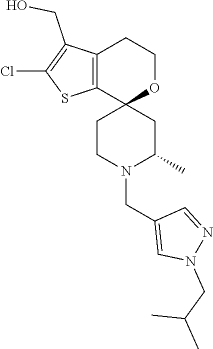 [SMILES: CC(C)Cn1cc(CN2CC[C@]3(C[C@@H]2C)OCCc2c3sc(Cl)c2CO)cn1] | 759 |
| 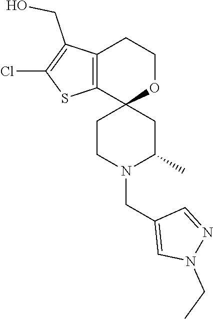 [SMILES: CCn1cc(CN2CC[C@]3(C[C@@H]2C)OCCc2c3sc(Cl)c2CO)cn1] | 760 |
| 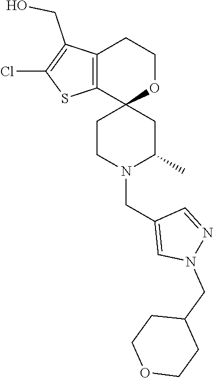 [SMILES: C[C@H]1C[C@@]2(CCN1Cc1cnn(CC3CCOCC3)c1)OCCc1c2sc(Cl)c1CO] | 761 |
| 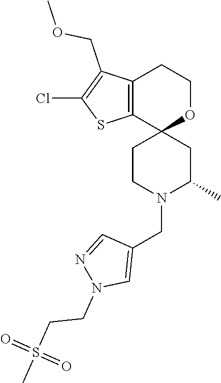 [SMILES: COCc1c(Cl)sc2c1CCO[C@@]21CCN(Cc2cnn(CCS(C)(=O)=O)c2)[C@@H](C)C1] | 762 |
| 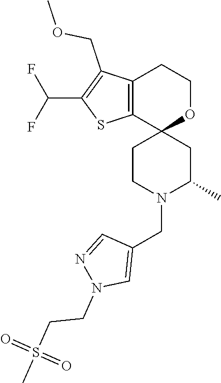 [SMILES: COCc1c(C(F)F)sc2c1CCO[C@@]21CCN(Cc2cnn(CCS(C)(=O)=O)c2)[C@@H](C)C1] | 763 |
| 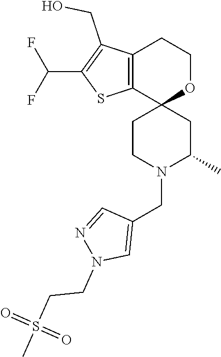 [SMILES: C[C@H]1C[C@@]2(CCN1Cc1cnn(CCS(C)(=O)=O)c1)OCCc1c2sc(C(F)F)c1CO] | 764 |
| 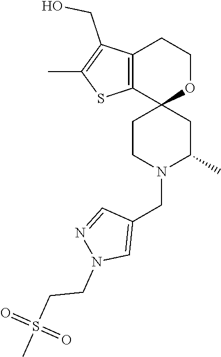 [SMILES: Cc1sc2c(c1CO)CCO[C@@]21CCN(Cc2cnn(CCS(C)(=O)=O)c2)[C@@H](C)C1] | 765 |
| 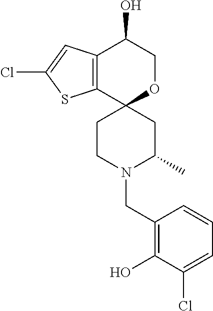 [SMILES: C[C@H]1C[C@@]2(CCN1Cc1cccc(Cl)c1O)OC[C@H](O)c1cc(Cl)sc12] | 766 |
| 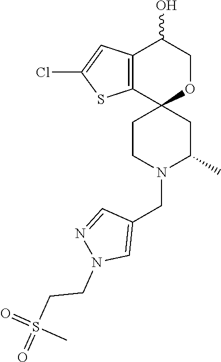 | 767 |
| 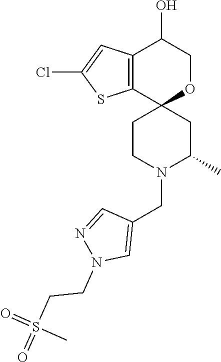 | 768 |
| 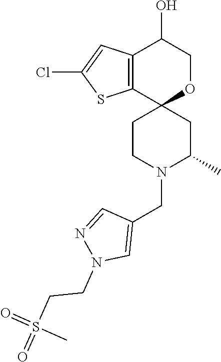 | 769 |
| 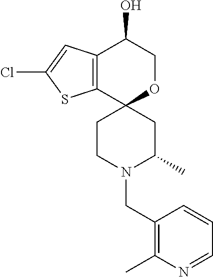 [SMILES: Cc1ncccc1CN1CC[C@]2(C[C@@H]1C)OC[C@H](O)c1cc(Cl)sc12] | 770 |
| 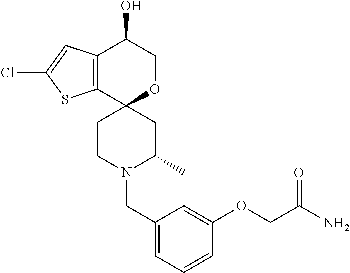 [SMILES: C[C@H]1C[C@@]2(CCN1Cc1cccc(OCC(N)=O)c1)OC[C@H](O)c1cc(Cl)sc12] | 771 |
| 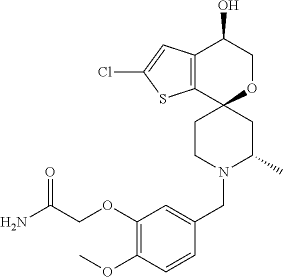 [SMILES: COc1ccc(CN2CC[C@]3(C[C@@H]2C)OC[C@H](O)c2cc(Cl)sc23)cc1OCC(N)=O] | 772 |
| 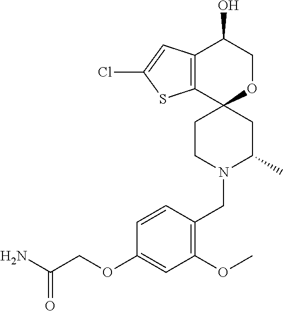 [SMILES: COc1cc(OCC(N)=O)ccc1CN1CC[C@]2(C[C@@H]1C)OC[C@H](O)c1cc(Cl)sc12] | 773 |
| 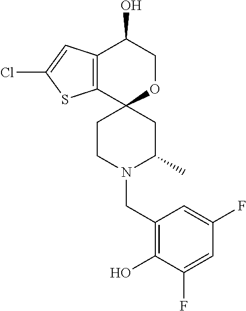 [SMILES: C[C@H]1C[C@@]2(CCN1Cc1cc(F)cc(F)c1O)OC[C@H](O)c1cc(Cl)sc12] | 774 |
| 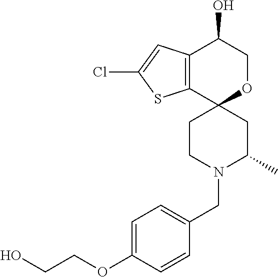 [SMILES: C[C@H]1C[C@@]2(CCN1Cc1ccc(OCCO)cc1)OC[C@H](O)c1cc(Cl)sc12] | 775 |
| 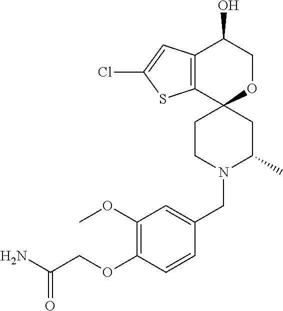 [SMILES: COc1cc(CN2CC[C@]3(C[C@@H]2C)OC[C@H](O)c2cc(Cl)sc23)ccc1OCC(N)=O] | 776 |
| 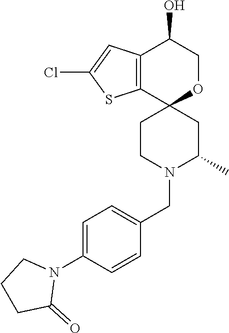 [SMILES: C[C@H]1C[C@@]2(CCN1Cc1ccc(N3CCCC3=O)cc1)OC[C@H](O)c1cc(Cl)sc12] | 777 |
| 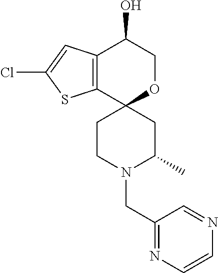 [SMILES: C[C@H]1C[C@@]2(CCN1Cc1cnccn1)OC[C@H](O)c1cc(Cl)sc12] | 778 |
| 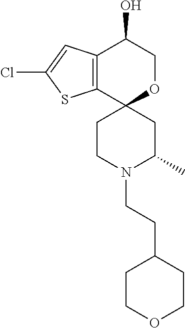 [SMILES: C[C@H]1C[C@@]2(CCN1CCC1CCOCC1)OC[C@H](O)c1cc(Cl)sc12] | 779 |
| 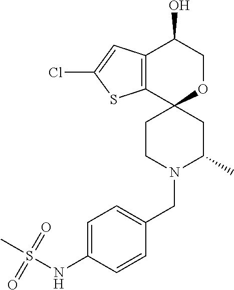 | 780 |
| 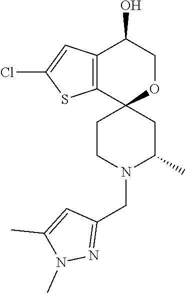 | 781 |
| 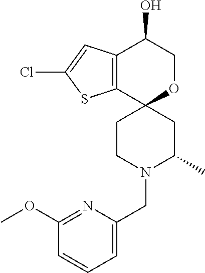 | 782 |
| 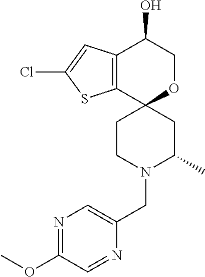 | 783 |
| 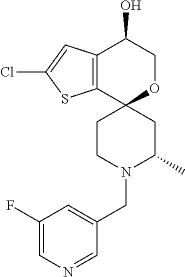 [SMILES: C[C@H]1C[C@@]2(CCN1Cc1cncc(F)c1)OC[C@H](O)c1cc(Cl)sc12] | 784 |
| 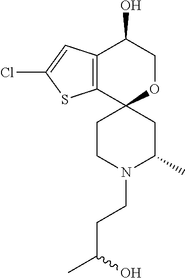 [SMILES: CC(O)CCN1CC[C@]2(C[C@@H]1C)OC[C@H](O)c1cc(Cl)sc12] | 785 |
| 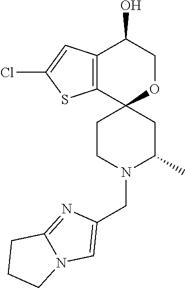 [SMILES: C[C@H]1C[C@@]2(CCN1Cc1cn3c(n1)CCC3)OC[C@H](O)c1cc(Cl)sc12] | 786 |
| 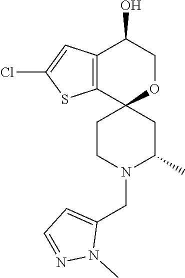 [SMILES: C[C@H]1C[C@@]2(CCN1Cc1ccnn1C)OC[C@H](O)c1cc(Cl)sc12] | 787 |
| 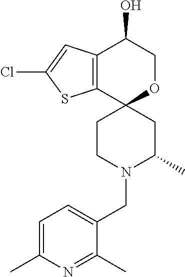 | 788 |
| 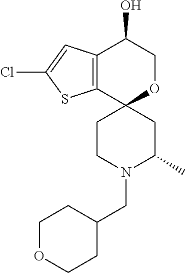 [SMILES: C[C@H]1C[C@@]2(CCN1CC1CCOCC1)OC[C@H](O)c1cc(Cl)sc12] | 789 |
| 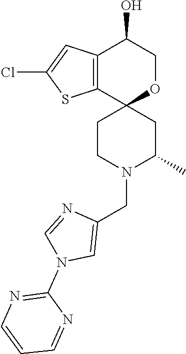 [SMILES: C[C@H]1C[C@@]2(CCN1Cc1cn(-c3ncccn3)cn1)OC[C@H](O)c1cc(Cl)sc12] | 790 |
| 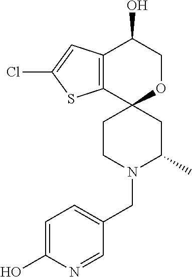 [SMILES: C[C@H]1C[C@@]2(CCN1Cc1ccc(O)nc1)OC[C@H](O)c1cc(Cl)sc12] | 791 |
| 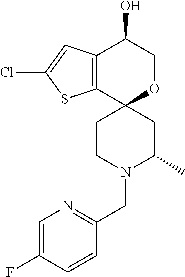 [SMILES: C[C@H]1C[C@@]2(CCN1Cc1ccc(F)cn1)OC[C@H](O)c1cc(Cl)sc12] | 792 |
| 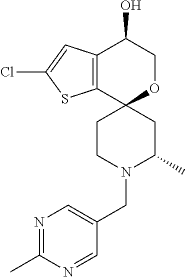 | 793 |
| 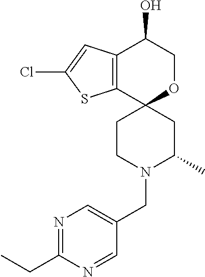 [SMILES: CCc1ncc(CN2CC[C@]3(C[C@@H]2C)OC[C@H](O)c2cc(Cl)sc23)cn1] | 794 |
| 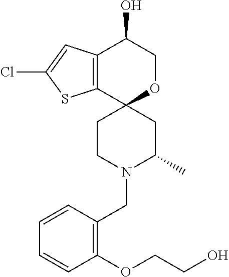 [SMILES: C[C@H]1C[C@@]2(CCN1Cc1ccccc1OCCO)OC[C@H](O)c1cc(Cl)sc12] | 795 |
| 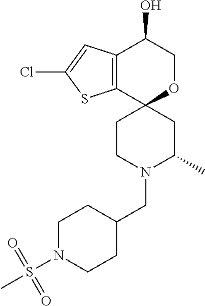 | 796 |
| 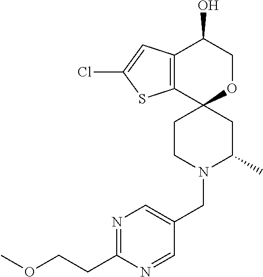 | 797 |
| 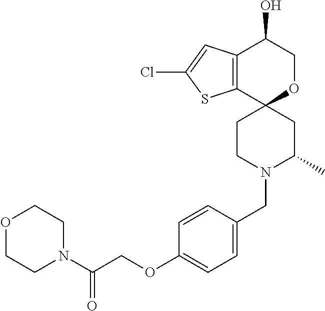 [SMILES: C[C@H]1C[C@@]2(CCN1Cc1ccc(OCC(=O)N3CCOCC3)cc1)OC[C@H](O)c1cc(Cl)sc12] | 798 |
| 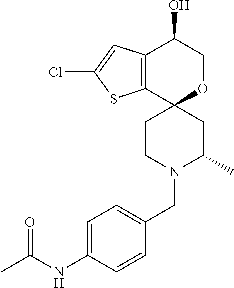 [SMILES: CC(=O)Nc1ccc(CN2CC[C@]3(C[C@@H]2C)OC[C@H](O)c2cc(Cl)sc23)cc1] | 799 |
| 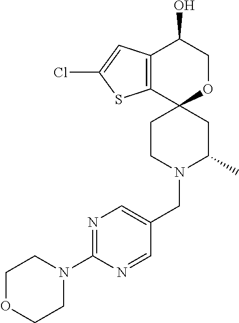 [SMILES: C[C@H]1C[C@@]2(CCN1Cc1cnc(N3CCOCC3)nc1)OC[C@H](O)c1cc(Cl)sc12] | 800 |
| 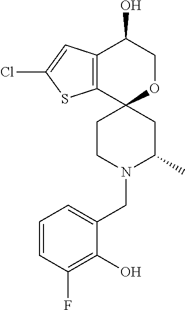 [SMILES: C[C@H]1C[C@@]2(CCN1Cc1cccc(F)c1O)OC[C@H](O)c1cc(Cl)sc12] | 801 |
| 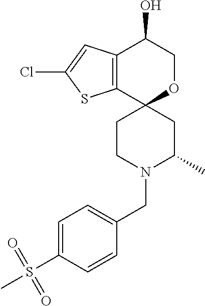 | 802 |
| 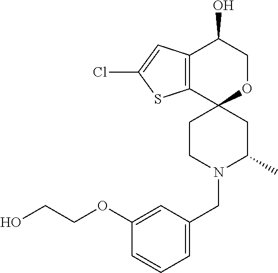 [SMILES: C[C@H]1C[C@@]2(CCN1Cc1cccc(OCCO)c1)OC[C@H](O)c1cc(Cl)sc12] | 803 |
| 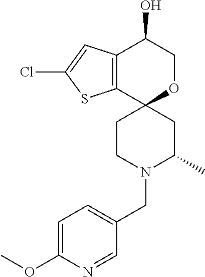 | 804 |
| 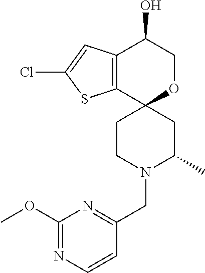 | 805 |
| 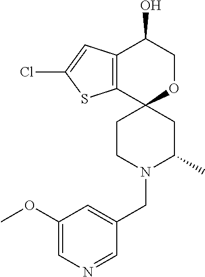 | 806 |
| 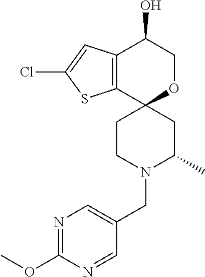 | 807 |
| 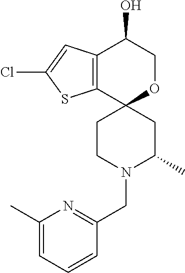 | 808 |
| 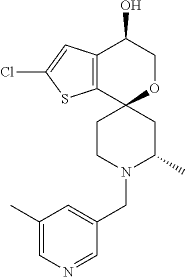 | 809 |
| 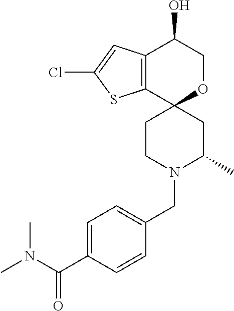 | 810 |
| 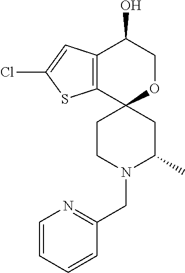 [SMILES: C[C@H]1C[C@@]2(CCN1Cc1ccccn1)OC[C@H](O)c1cc(Cl)sc12] | 811 |
| 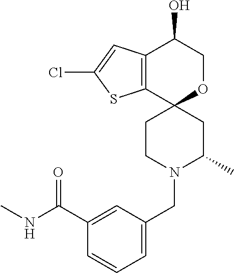 | 812 |
| 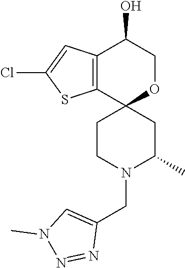 | 813 |
| 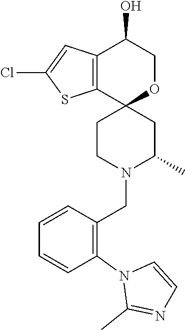 [SMILES: Cc1nccn1-c1ccccc1CN1CC[C@]2(C[C@@H]1C)OC[C@H](O)c1cc(Cl)sc12] | 814 |
| 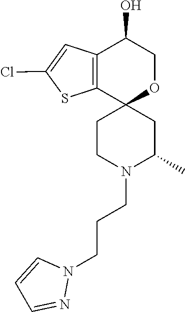 [SMILES: C[C@H]1C[C@@]2(CCN1CCCn1cccn1)OC[C@H](O)c1cc(Cl)sc12] | 815 |
| 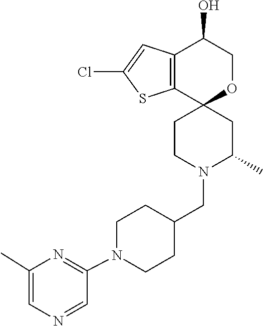 | 816 |
| 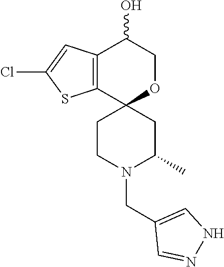 | 817 |
| 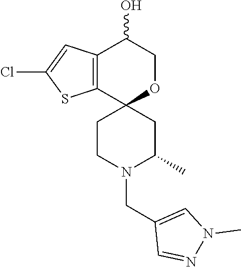 | 818 |
| 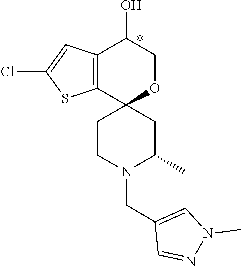 | 819 |
| 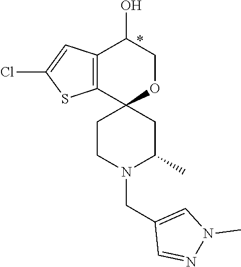 | 820 |
| 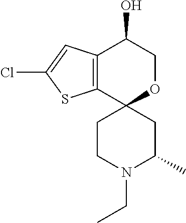 [SMILES: CCN1CC[C@]2(C[C@@H]1C)OC[C@H](O)c1cc(Cl)sc12] | 821 |
| 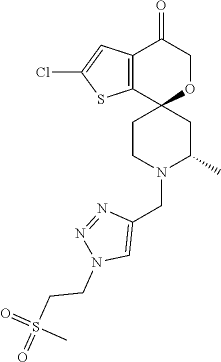 [SMILES: C[C@H]1C[C@@]2(CCN1Cc1cn(CCS(C)(=O)=O)nn1)OCC(=O)c1cc(Cl)sc12] | 822 |
| 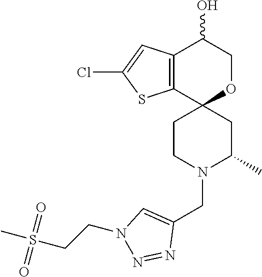 | 823 |
| 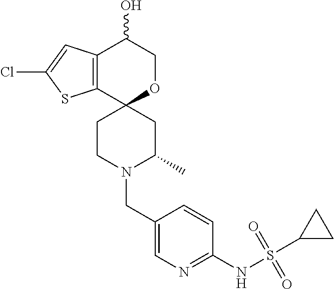 | 824 |
| 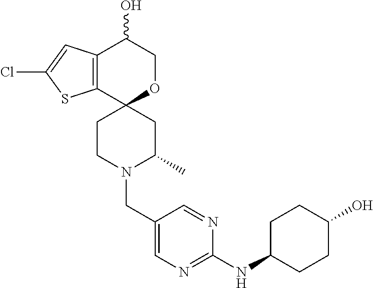 | 825 |
| 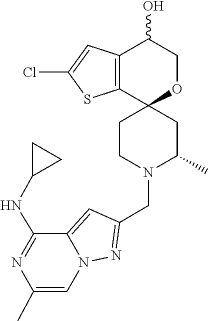 | 826 |
| 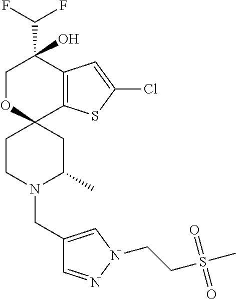 [SMILES: C[C@H]1C[C@@]2(CCN1Cc1cnn(CCS(C)(=O)=O)c1)OC[C@](O)(C(F)F)c1cc(Cl)sc12] | 827 |
| 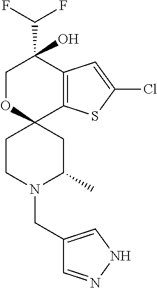 [SMILES: C[C@H]1C[C@@]2(CCN1Cc1cn[nH]c1)OC[C@](O)(C(F)F)c1cc(Cl)sc12] | 828 |
| 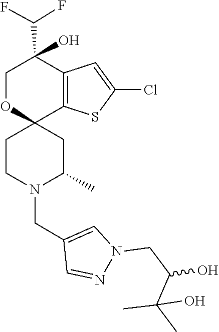 [SMILES: C[C@H]1C[C@@]2(CCN1Cc1cnn(CC(O)C(C)(C)O)c1)OC[C@](O)(C(F)F)c1cc(Cl)sc12] | 829 |
| 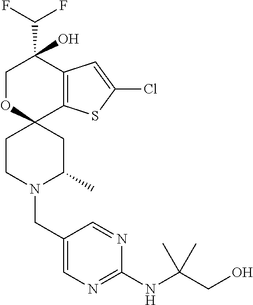 [SMILES: C[C@H]1C[C@@]2(CCN1Cc1cnc(NC(C)(C)CO)nc1)OC[C@](O)(C(F)F)c1cc(Cl)sc12] | 830 |
| 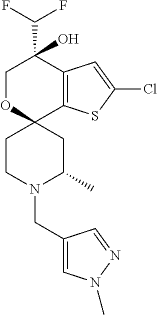 [SMILES: C[C@H]1C[C@@]2(CCN1Cc1cnn(C)c1)OC[C@](O)(C(F)F)c1cc(Cl)sc12] | 831 |
| 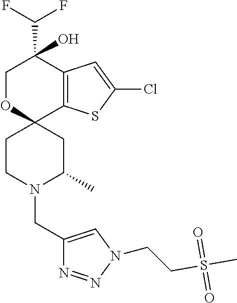 [SMILES: C[C@H]1C[C@@]2(CCN1Cc1cn(CCS(C)(=O)=O)nn1)OC[C@](O)(C(F)F)c1cc(Cl)sc12] | 832 |
| 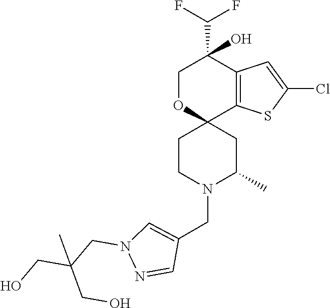 [SMILES: C[C@H]1C[C@@]2(CCN1Cc1cnn(CC(C)(CO)CO)c1)OC[C@](O)(C(F)F)c1cc(Cl)sc12] | 833 |
| 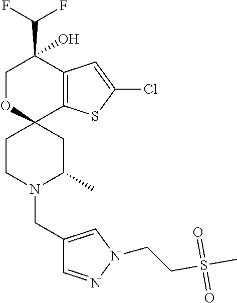 [SMILES: C[C@H]1C[C@@]2(CCN1Cc1cnn(CCS(C)(=O)=O)c1)OC[C@@](O)(C(F)F)c1cc(Cl)sc12] | 834 |
| 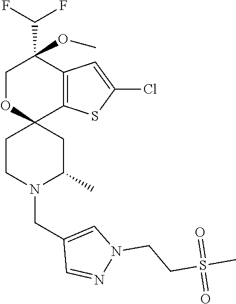 [SMILES: CO[C@]1(C(F)F)CO[C@@]2(CCN(Cc3cnn(CCS(C)(=O)=O)c3)[C@@H](C)C2)c2sc(Cl)cc21] | 835 |
| 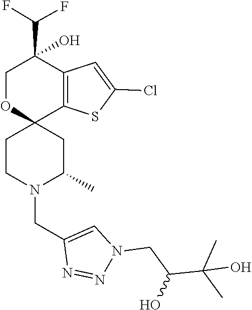 [SMILES: C[C@H]1C[C@@]2(CCN1Cc1cn(CC(O)C(C)(C)O)nn1)OC[C@@](O)(C(F)F)c1cc(Cl)sc12] | 836 |
| 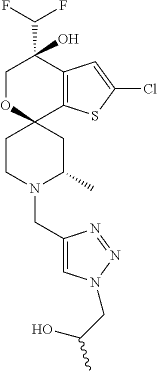 [SMILES: CC(O)Cn1cc(CN2CC[C@]3(C[C@@H]2C)OC[C@](O)(C(F)F)c2cc(Cl)sc23)nn1] | 837 |
| 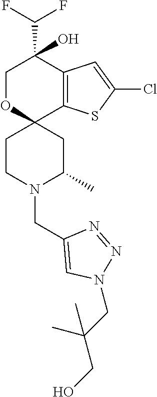 [SMILES: C[C@H]1C[C@@]2(CCN1Cc1cn(CC(C)(C)CO)nn1)OC[C@](O)(C(F)F)c1cc(Cl)sc12] | 838 |
| 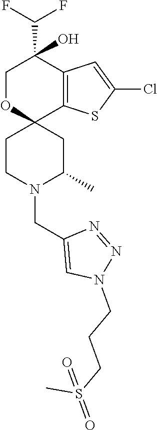 [SMILES: C[C@H]1C[C@@]2(CCN1Cc1cn(CCCS(C)(=O)=O)nn1)OC[C@](O)(C(F)F)c1cc(Cl)sc12] | 839 |
| 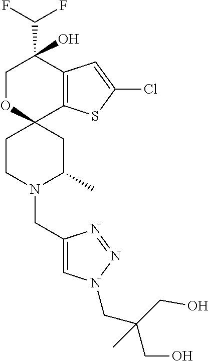 [SMILES: C[C@H]1C[C@@]2(CCN1Cc1cn(CC(C)(CO)CO)nn1)OC[C@](O)(C(F)F)c1cc(Cl)sc12] | 840 |
| 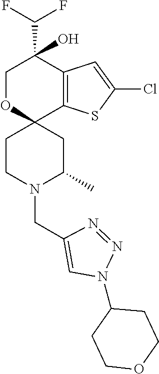 [SMILES: C[C@H]1C[C@@]2(CCN1Cc1cn(C3CCOCC3)nn1)OC[C@](O)(C(F)F)c1cc(Cl)sc12] | 841 |
| 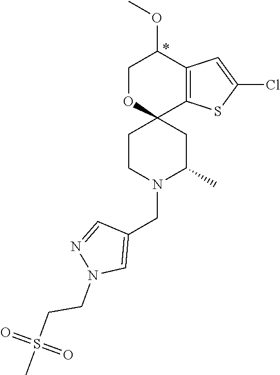 | 842 |
| 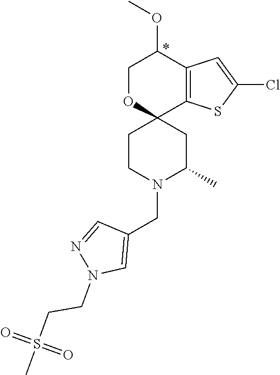 | 843 |
| 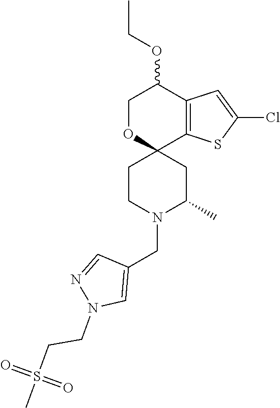 | 844 |
| 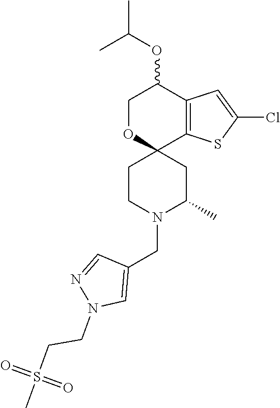 | 845 |
| 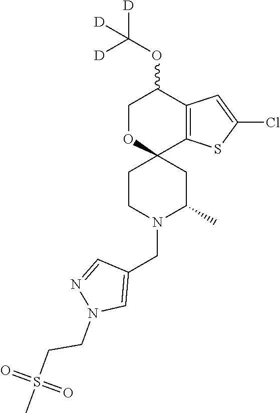 | 846 |
| 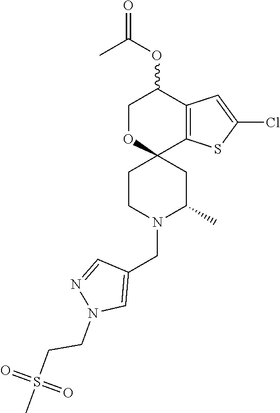 | 847 |
| 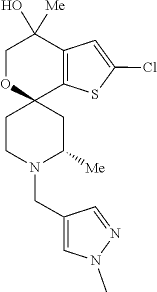 | 848 |
| 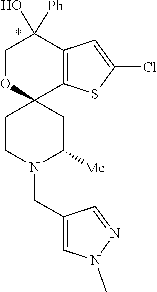 | 849 |
| 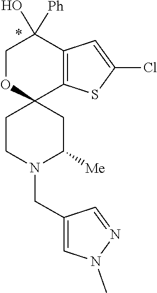 | 850 |
| 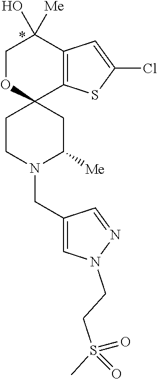 | 851 |
| 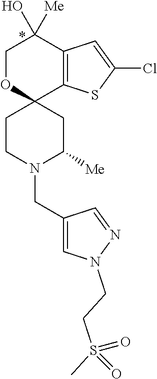 | 852 |
| 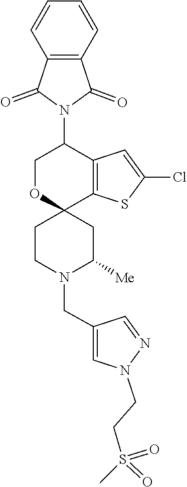 | 853 |
| 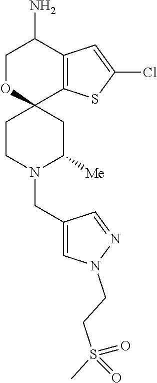 | 854 |
| 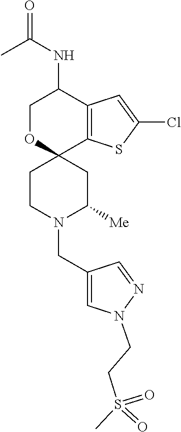 | 855 |
| 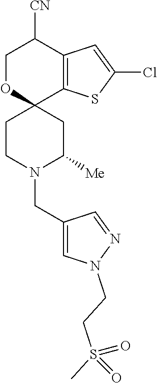 | 856 |
| 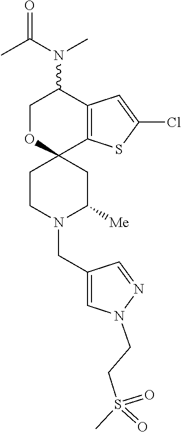 | 857 |
| 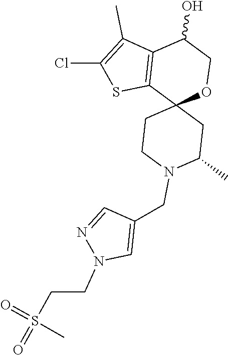 | 858 |
| 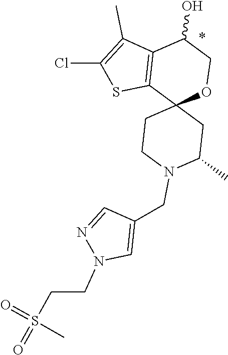 | 859 |
| 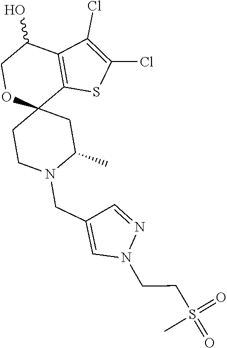 | 860 |
| 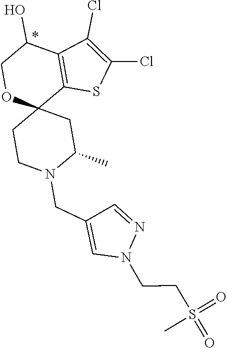 | 861 |
| 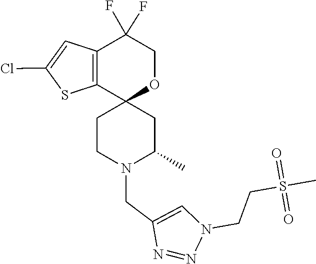 [SMILES: C[C@H]1C[C@@]2(CCN1Cc1cn(CCS(C)(=O)=O)nn1)OCC(F)(F)c1cc(Cl)sc12] | 862 |
| 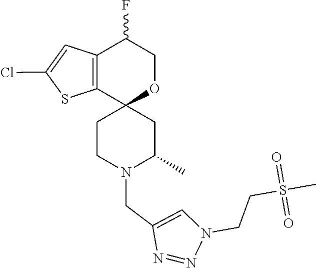 | 863 |
| 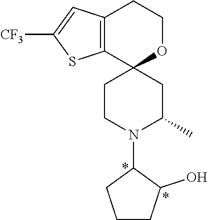 [SMILES: C[C@H]1C[C@@]2(CCN1C1CCCC1O)OCCc1cc(C(F)(F)F)sc12] | 864 |
| 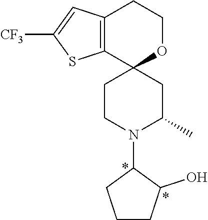 [SMILES: C[C@H]1C[C@@]2(CCN1C1CCCC1O)OCCc1cc(C(F)(F)F)sc12] | 865 |
| 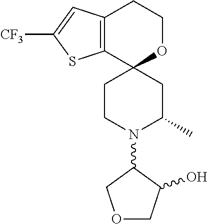 [SMILES: C[C@H]1C[C@@]2(CCN1C1COCC1O)OCCc1cc(C(F)(F)F)sc12] | 866 |
| 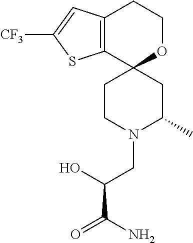 [SMILES: C[C@H]1C[C@@]2(CCN1C[C@H](O)C(N)=O)OCCc1cc(C(F)(F)F)sc12] | 867 |
| 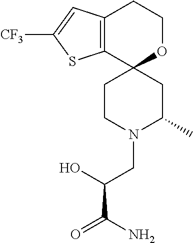 [SMILES: C[C@H]1C[C@@]2(CCN1C[C@H](O)C(N)=O)OCCc1cc(C(F)(F)F)sc12] | 868 |
| 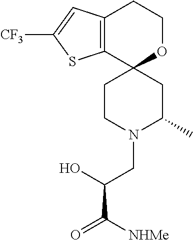 [SMILES: CNC(=O)[C@@H](O)CN1CC[C@]2(C[C@@H]1C)OCCc1cc(C(F)(F)F)sc12] | 869 |
| 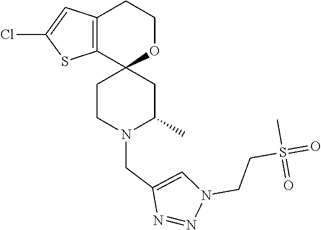 [SMILES: C[C@H]1C[C@@]2(CCN1Cc1cn(CCS(C)(=O)=O)nn1)OCCc1cc(Cl)sc12] | 870 |
| 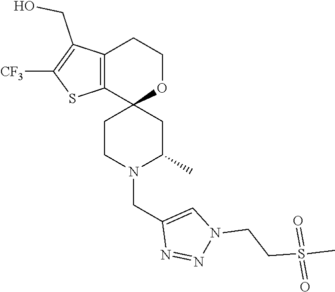 [SMILES: C[C@H]1C[C@@]2(CCN1Cc1cn(CCS(C)(=O)=O)nn1)OCCc1c2sc(C(F)(F)F)c1CO] | 871 |
| 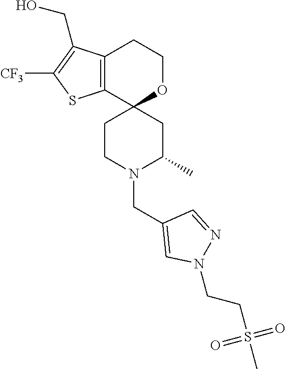 [SMILES: C[C@H]1C[C@@]2(CCN1Cc1cnn(CCS(C)(=O)=O)c1)OCCc1c2sc(C(F)(F)F)c1CO] | 872 |
| 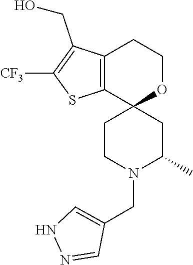 [SMILES: C[C@H]1C[C@@]2(CCN1Cc1cn[nH]c1)OCCc1c2sc(C(F)(F)F)c1CO] | 873 |
| 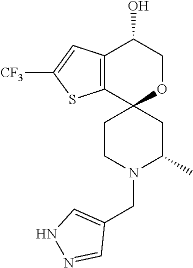 [SMILES: C[C@H]1C[C@@]2(CCN1Cc1cn[nH]c1)OC[C@@H](O)c1cc(C(F)(F)F)sc12] | 874 |
| 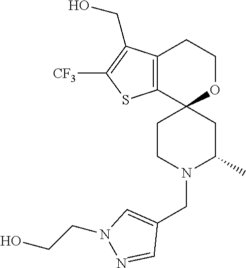 [SMILES: C[C@H]1C[C@@]2(CCN1Cc1cnn(CCO)c1)OCCc1c2sc(C(F)(F)F)c1CO] | 875 |
| 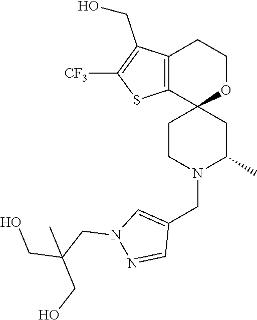 [SMILES: C[C@H]1C[C@@]2(CCN1Cc1cnn(CC(C)(CO)CO)c1)OCCc1c2sc(C(F)(F)F)c1CO] | 876 |
| 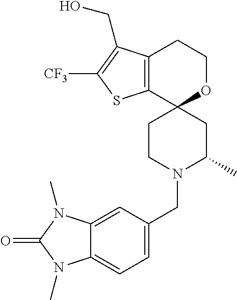 [SMILES: C[C@H]1C[C@@]2(CCN1Cc1ccc3c(c1)n(C)c(=O)n3C)OCCc1c2sc(C(F)(F)F)c1CO] | 877 |
| 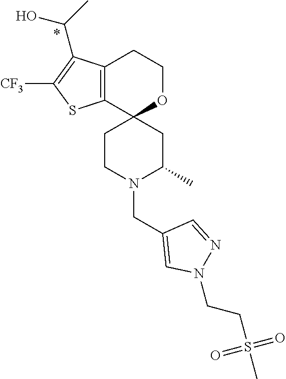 [SMILES: CC(O)c1c(C(F)(F)F)sc2c1CCO[C@@]21CCN(Cc2cnn(CCS(C)(=O)=O)c2)[C@@H](C)C1] | 878 |
| 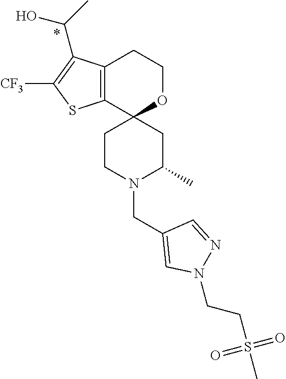 [SMILES: CC(O)c1c(C(F)(F)F)sc2c1CCO[C@@]21CCN(Cc2cnn(CCS(C)(=O)=O)c2)[C@@H](C)C1] | 879 |
| 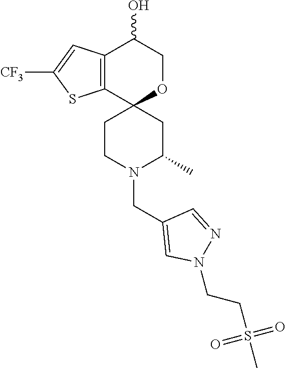 | 880 |
| 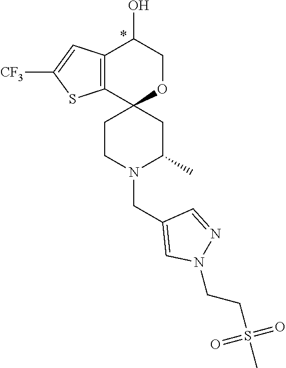 | 881 |
| 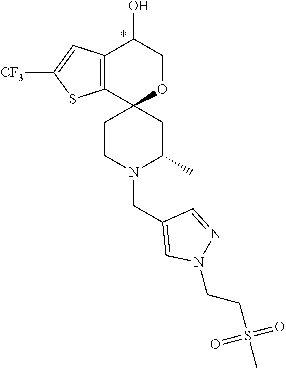 | 882 |
| 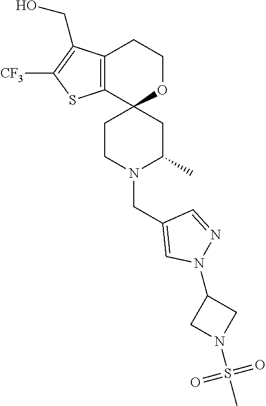 [SMILES: C[C@H]1C[C@@]2(CCN1Cc1cnn(C3CN(S(C)(=O)=O)C3)c1)OCCc1c2sc(C(F)(F)F)c1CO] | 883 |
| 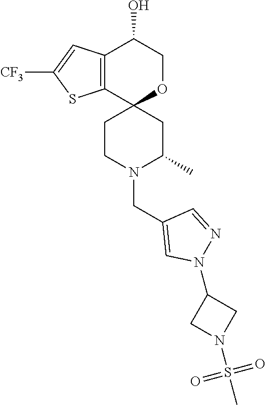 [SMILES: C[C@H]1C[C@@]2(CCN1Cc1cnn(C3CN(S(C)(=O)=O)C3)c1)OC[C@@H](O)c1cc(C(F)(F)F)sc12] | 884 |
| 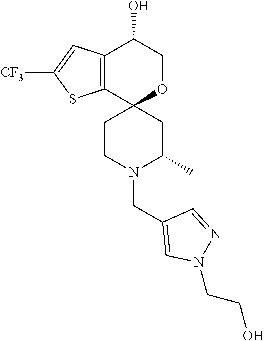 [SMILES: C[C@H]1C[C@@]2(CCN1Cc1cnn(CCO)c1)OC[C@@H](O)c1cc(C(F)(F)F)sc12] | 885 |
| 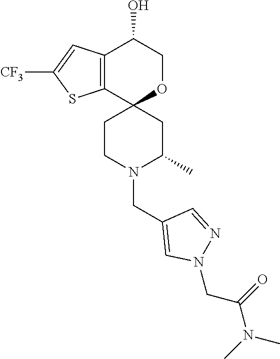 [SMILES: C[C@H]1C[C@@]2(CCN1Cc1cnn(CC(=O)N(C)C)c1)OC[C@@H](O)c1cc(C(F)(F)F)sc12] | 886 |
| 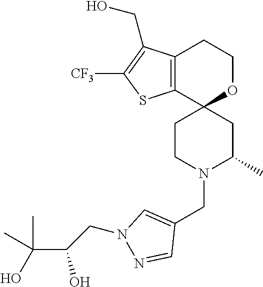 [SMILES: C[C@H]1C[C@@]2(CCN1Cc1cnn(C[C@H](O)C(C)(C)O)c1)OCCc1c2sc(C(F)(F)F)c1CO] | 887 |
| 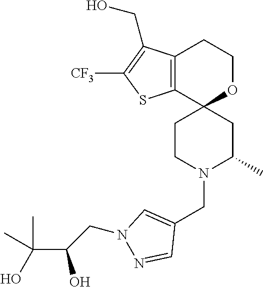 [SMILES: C[C@H]1C[C@@]2(CCN1Cc1cnn(C[C@@H](O)C(C)(C)O)c1)OCCc1c2sc(C(F)(F)F)c1CO] | 888 |
| 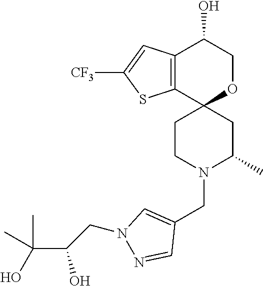 [SMILES: C[C@H]1C[C@@]2(CCN1Cc1cnn(C[C@H](O)C(C)(C)O)c1)OC[C@@H](O)c1cc(C(F)(F)F)sc12] | 889 |
| 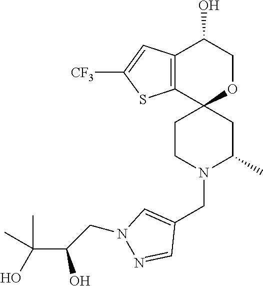 [SMILES: C[C@H]1C[C@@]2(CCN1Cc1cnn(C[C@@H](O)C(C)(C)O)c1)OC[C@@H](O)c1cc(C(F)(F)F)sc12] | 890 |
| 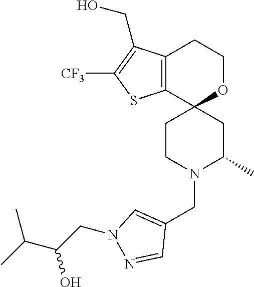 [SMILES: CC(C)C(O)Cn1cc(CN2CC[C@]3(C[C@@H]2C)OCCc2c3sc(C(F)(F)F)c2CO)cn1] | 891 |
| 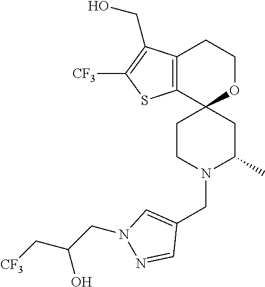 [SMILES: C[C@H]1C[C@@]2(CCN1Cc1cnn(CC(O)CC(F)(F)F)c1)OCCc1c2sc(C(F)(F)F)c1CO] | 892 |
| 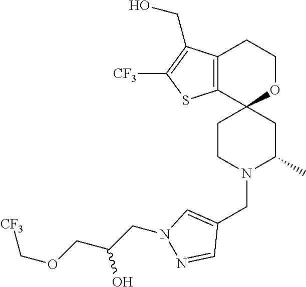 [SMILES: C[C@H]1C[C@@]2(CCN1Cc1cnn(CC(O)COCC(F)(F)F)c1)OCCc1c2sc(C(F)(F)F)c1CO] | 893 |
| 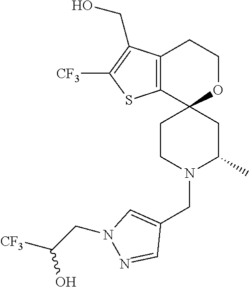 [SMILES: C[C@H]1C[C@@]2(CCN1Cc1cnn(CC(O)C(F)(F)F)c1)OCCc1c2sc(C(F)(F)F)c1CO] | 894 |
| 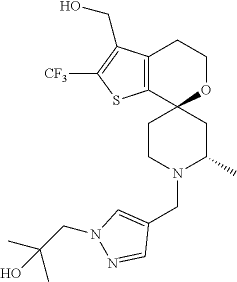 [SMILES: C[C@H]1C[C@@]2(CCN1Cc1cnn(CC(C)(C)O)c1)OCCc1c2sc(C(F)(F)F)c1CO] | 895 |
| 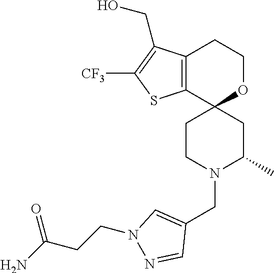 [SMILES: C[C@H]1C[C@@]2(CCN1Cc1cnn(CCC(N)=O)c1)OCCc1c2sc(C(F)(F)F)c1CO] | 896 |
| 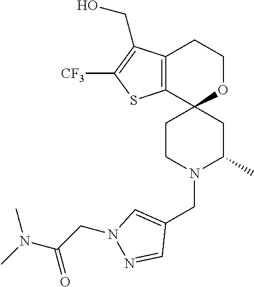 | 897 |
| 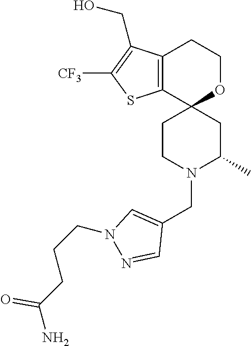 [SMILES: C[C@H]1C[C@@]2(CCN1Cc1cnn(CCCC(N)=O)c1)OCCc1c2sc(C(F)(F)F)c1CO] | 898 |
| 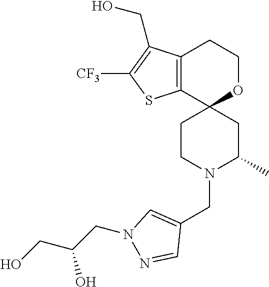 [SMILES: C[C@H]1C[C@@]2(CCN1Cc1cnn(C[C@H](O)CO)c1)OCCc1c2sc(C(F)(F)F)c1CO] | 899 |
| 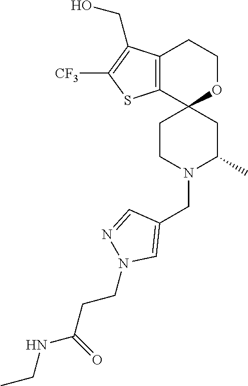 [SMILES: CCNC(=O)CCn1cc(CN2CC[C@]3(C[C@@H]2C)OCCc2c3sc(C(F)(F)F)c2CO)cn1] | 900 |
| 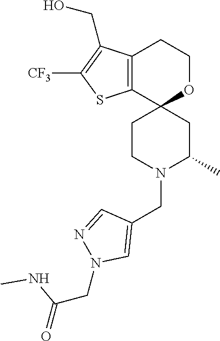 | 901 |
| 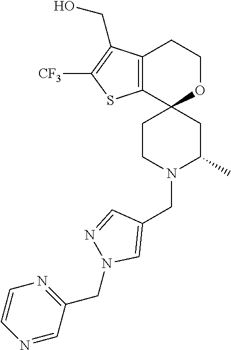 [SMILES: C[C@H]1C[C@@]2(CCN1Cc1cnn(Cc3cnccn3)c1)OCCc1c2sc(C(F)(F)F)c1CO] | 902 |
| 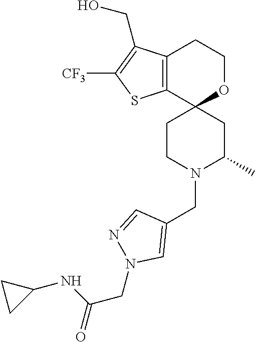 [SMILES: C[C@H]1C[C@@]2(CCN1Cc1cnn(CC(=O)NC3CC3)c1)OCCc1c2sc(C(F)(F)F)c1CO] | 903 |
| 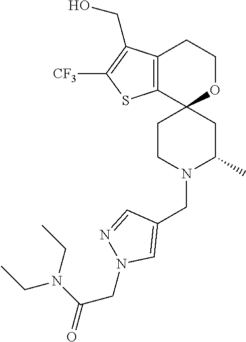 [SMILES: CCN(CC)C(=O)Cn1cc(CN2CC[C@]3(C[C@@H]2C)OCCc2c3sc(C(F)(F)F)c2CO)cn1] | 904 |
| 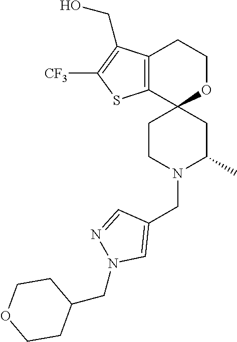 [SMILES: C[C@H]1C[C@@]2(CCN1Cc1cnn(CC3CCOCC3)c1)OCCc1c2sc(C(F)(F)F)c1CO] | 905 |
| 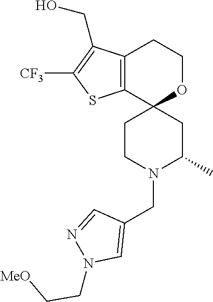 [SMILES: COCCn1cc(CN2CC[C@]3(C[C@@H]2C)OCCc2c3sc(C(F)(F)F)c2CO)cn1] | 906 |
| 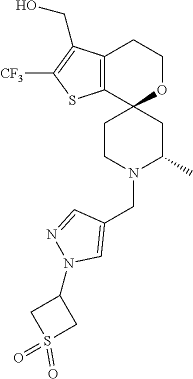 [SMILES: C[C@H]1C[C@@]2(CCN1Cc1cnn(C3CS(=O)(=O)C3)c1)OCCc1c2sc(C(F)(F)F)c1CO] | 907 |
| 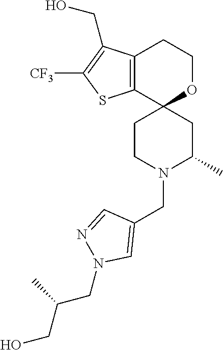 [SMILES: C[C@@H](CO)Cn1cc(CN2CC[C@]3(C[C@@H]2C)OCCc2c3sc(C(F)(F)F)c2CO)cn1] | 908 |
| 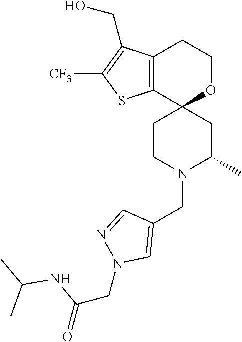 [SMILES: CC(C)NC(=O)Cn1cc(CN2CC[C@]3(C[C@@H]2C)OCCc2c3sc(C(F)(F)F)c2CO)cn1] | 909 |
| 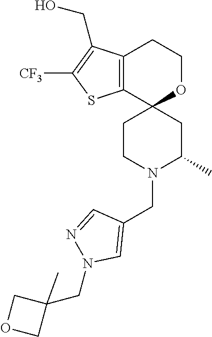 [SMILES: C[C@H]1C[C@@]2(CCN1Cc1cnn(CC3(C)COC3)c1)OCCc1c2sc(C(F)(F)F)c1CO] | 910 |
| 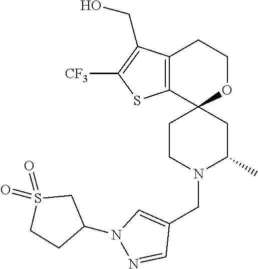 [SMILES: C[C@H]1C[C@@]2(CCN1Cc1cnn(C3CCS(=O)(=O)C3)c1)OCCc1c2sc(C(F)(F)F)c1CO] | 911 |
| 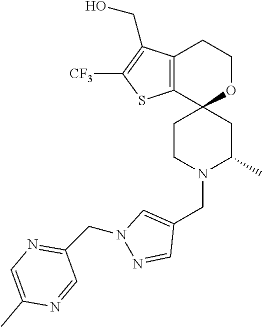 | 912 |
| 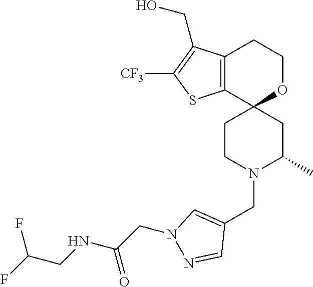 [SMILES: C[C@H]1C[C@@]2(CCN1Cc1cnn(CC(=O)NCC(F)F)c1)OCCc1c2sc(C(F)(F)F)c1CO] | 913 |
| 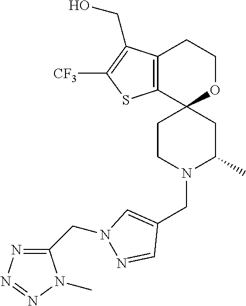 [SMILES: C[C@H]1C[C@@]2(CCN1Cc1cnn(Cc3nnnn3C)c1)OCCc1c2sc(C(F)(F)F)c1CO] | 914 |
| 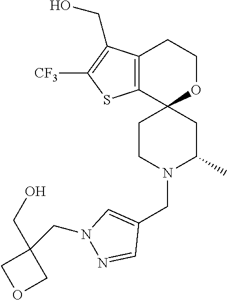 [SMILES: C[C@H]1C[C@@]2(CCN1Cc1cnn(CC3(CO)COC3)c1)OCCc1c2sc(C(F)(F)F)c1CO] | 915 |
| 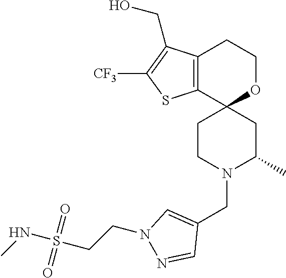 | 916 |
| 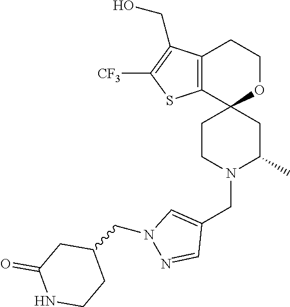 [SMILES: C[C@H]1C[C@@]2(CCN1Cc1cnn(CC3CCNC(=O)C3)c1)OCCc1c2sc(C(F)(F)F)c1CO] | 917 |
| 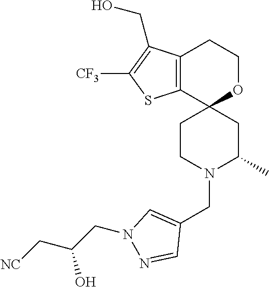 [SMILES: C[C@H]1C[C@@]2(CCN1Cc1cnn(C[C@H](O)CC#N)c1)OCCc1c2sc(C(F)(F)F)c1CO] | 918 |
| 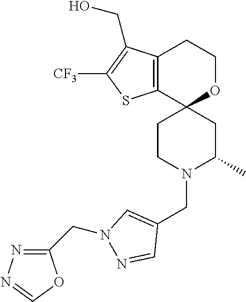 [SMILES: C[C@H]1C[C@@]2(CCN1Cc1cnn(Cc3nnco3)c1)OCCc1c2sc(C(F)(F)F)c1CO] | 919 |
| 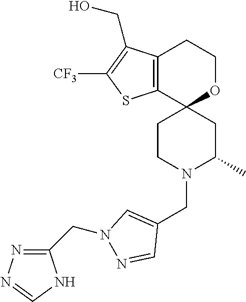 [SMILES: C[C@H]1C[C@@]2(CCN1Cc1cnn(Cc3nnc[nH]3)c1)OCCc1c2sc(C(F)(F)F)c1CO] | 920 |
| 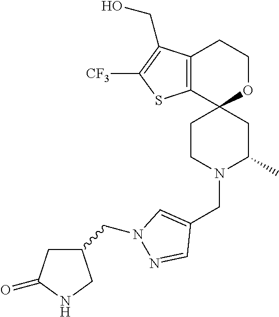 [SMILES: C[C@H]1C[C@@]2(CCN1Cc1cnn(CC3CNC(=O)C3)c1)OCCc1c2sc(C(F)(F)F)c1CO] | 921 |
| 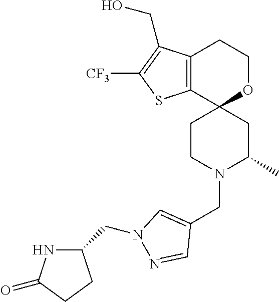 [SMILES: C[C@H]1C[C@@]2(CCN1Cc1cnn(C[C@@H]3CCC(=O)N3)c1)OCCc1c2sc(C(F)(F)F)c1CO] | 922 |
| 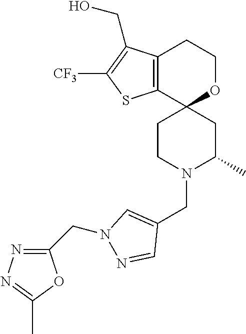 [SMILES: Cc1nnc(Cn2cc(CN3CC[C@]4(C[C@@H]3C)OCCc3c4sc(C(F)(F)F)c3CO)cn2)o1] | 923 |
| 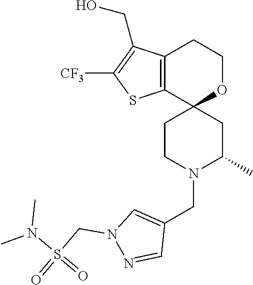 | 924 |
| 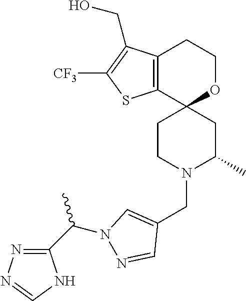 [SMILES: CC(c1nnc[nH]1)n1cc(CN2CC[C@]3(C[C@@H]2C)OCCc2c3sc(C(F)(F)F)c2CO)cn1] | 925 |
| 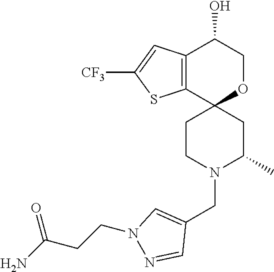 [SMILES: C[C@H]1C[C@@]2(CCN1Cc1cnn(CCC(N)=O)c1)OC[C@@H](O)c1cc(C(F)(F)F)sc12] | 926 |
| 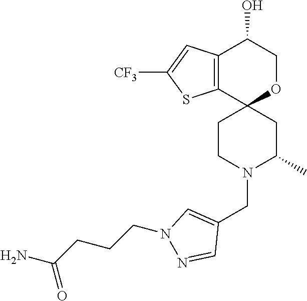 [SMILES: C[C@H]1C[C@@]2(CCN1Cc1cnn(CCCC(N)=O)c1)OC[C@@H](O)c1cc(C(F)(F)F)sc12] | 927 |
| 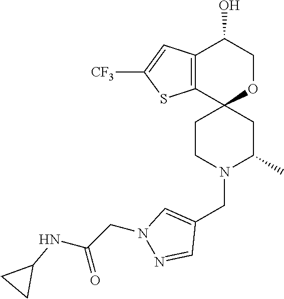 [SMILES: C[C@H]1C[C@@]2(CCN1Cc1cnn(CC(=O)NC3CC3)c1)OC[C@@H](O)c1cc(C(F)(F)F)sc12] | 928 |
| 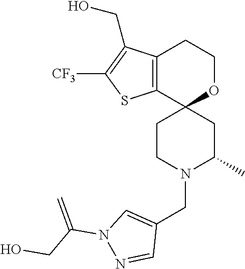 [SMILES: C=C(CO)n1cc(CN2CC[C@]3(C[C@@H]2C)OCCc2c3sc(C(F)(F)F)c2CO)cn1] | 929 |
| 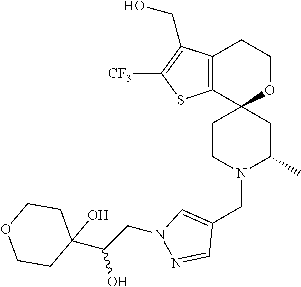 [SMILES: C[C@H]1C[C@@]2(CCN1Cc1cnn(CC(O)C3(O)CCOCC3)c1)OCCc1c2sc(C(F)(F)F)c1CO] | 930 |
| 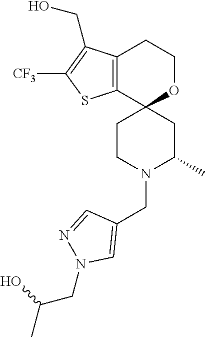 [SMILES: CC(O)Cn1cc(CN2CC[C@]3(C[C@@H]2C)OCCc2c3sc(C(F)(F)F)c2CO)cn1] | 931 |
| 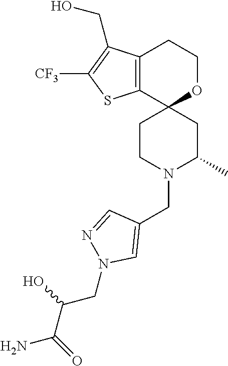 [SMILES: C[C@H]1C[C@@]2(CCN1Cc1cnn(CC(O)C(N)=O)c1)OCCc1c2sc(C(F)(F)F)c1CO] | 932 |
| 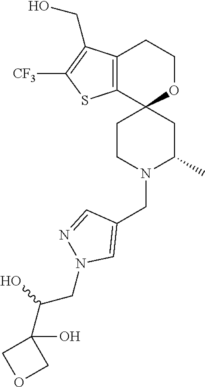 [SMILES: C[C@H]1C[C@@]2(CCN1Cc1cnn(CC(O)C3(O)COC3)c1)OCCc1c2sc(C(F)(F)F)c1CO] | 933 |
| 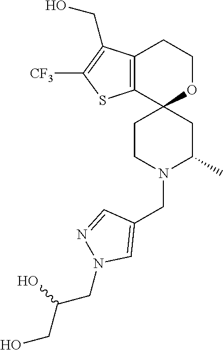 [SMILES: C[C@H]1C[C@@]2(CCN1Cc1cnn(CC(O)CO)c1)OCCc1c2sc(C(F)(F)F)c1CO] | 934 |
| 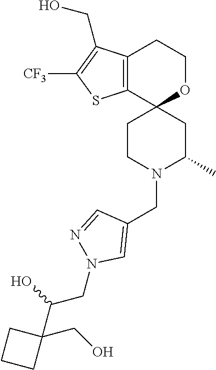 [SMILES: C[C@H]1C[C@@]2(CCN1Cc1cnn(CC(O)C3(CO)CCC3)c1)OCCc1c2sc(C(F)(F)F)c1CO] | 935 |
| 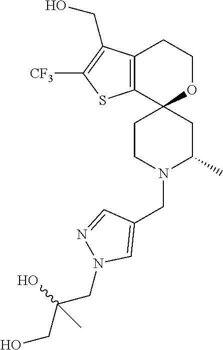 [SMILES: C[C@H]1C[C@@]2(CCN1Cc1cnn(CC(C)(O)CO)c1)OCCc1c2sc(C(F)(F)F)c1CO] | 936 |
| 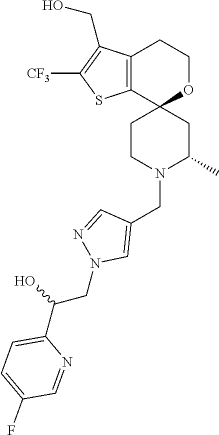 [SMILES: C[C@H]1C[C@@]2(CCN1Cc1cnn(CC(O)c3ccc(F)cn3)c1)OCCc1c2sc(C(F)(F)F)c1CO] | 937 |
| 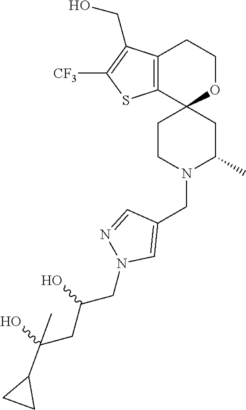 [SMILES: C[C@H]1C[C@@]2(CCN1Cc1cnn(CC(O)CC(C)(O)C3CC3)c1)OCCc1c2sc(C(F)(F)F)c1CO] | 938 |
| 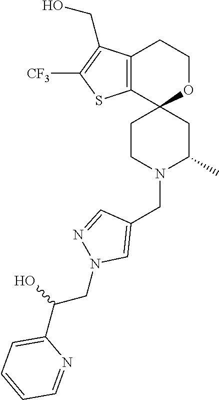 [SMILES: C[C@H]1C[C@@]2(CCN1Cc1cnn(CC(O)c3ccccn3)c1)OCCc1c2sc(C(F)(F)F)c1CO] | 939 |
| 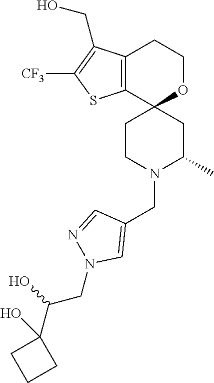 [SMILES: C[C@H]1C[C@@]2(CCN1Cc1cnn(CC(O)C3(O)CCC3)c1)OCCc1c2sc(C(F)(F)F)c1CO] | 940 |
| 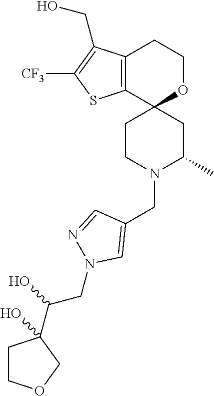 [SMILES: C[C@H]1C[C@@]2(CCN1Cc1cnn(CC(O)C3(O)CCOC3)c1)OCCc1c2sc(C(F)(F)F)c1CO] | 941 |
| 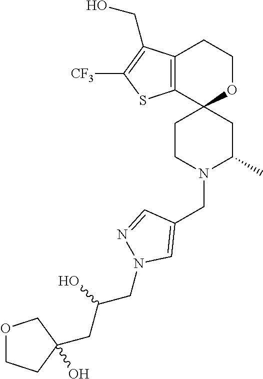 [SMILES: C[C@H]1C[C@@]2(CCN1Cc1cnn(CC(O)CC3(O)CCOC3)c1)OCCc1c2sc(C(F)(F)F)c1CO] | 942 |
| 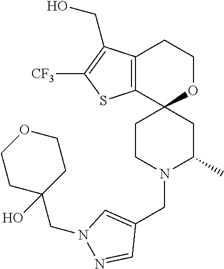 [SMILES: C[C@H]1C[C@@]2(CCN1Cc1cnn(CC3(O)CCOCC3)c1)OCCc1c2sc(C(F)(F)F)c1CO] | 943 |
| 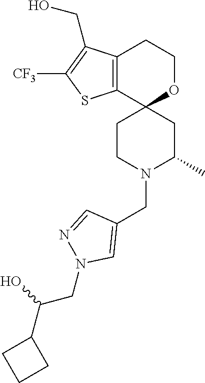 [SMILES: C[C@H]1C[C@@]2(CCN1Cc1cnn(CC(O)C3CCC3)c1)OCCc1c2sc(C(F)(F)F)c1CO] | 944 |
| 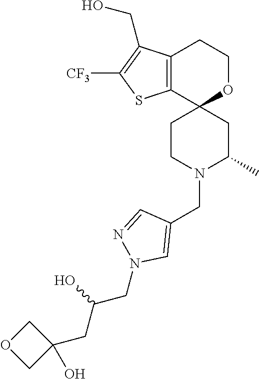 [SMILES: C[C@H]1C[C@@]2(CCN1Cc1cnn(CC(O)CC3(O)COC3)c1)OCCc1c2sc(C(F)(F)F)c1CO] | 945 |
| 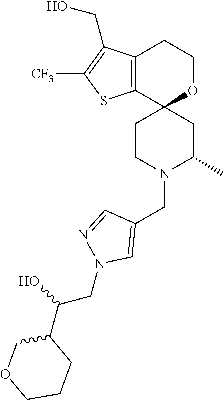 [SMILES: C[C@H]1C[C@@]2(CCN1Cc1cnn(CC(O)C3CCCOC3)c1)OCCc1c2sc(C(F)(F)F)c1CO] | 946 |
| 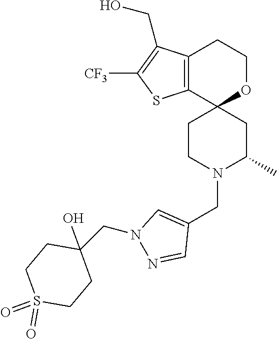 [SMILES: C[C@H]1C[C@@]2(CCN1Cc1cnn(CC3(O)CCS(=O)(=O)CC3)c1)OCCc1c2sc(C(F)(F)F)c1CO] | 947 |
| 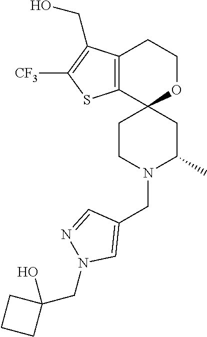 [SMILES: C[C@H]1C[C@@]2(CCN1Cc1cnn(CC3(O)CCC3)c1)OCCc1c2sc(C(F)(F)F)c1CO] | 948 |
| 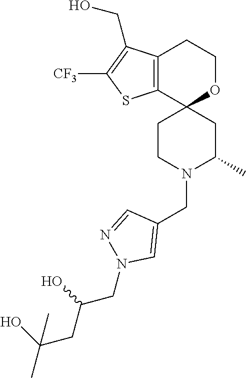 [SMILES: C[C@H]1C[C@@]2(CCN1Cc1cnn(CC(O)CC(C)(C)O)c1)OCCc1c2sc(C(F)(F)F)c1CO] | 949 |
| 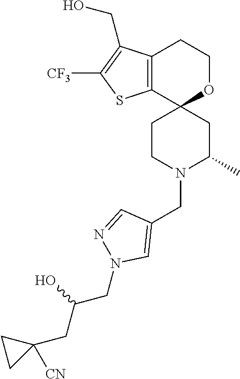 [SMILES: C[C@H]1C[C@@]2(CCN1Cc1cnn(CC(O)CC3(C#N)CC3)c1)OCCc1c2sc(C(F)(F)F)c1CO] | 950 |
| 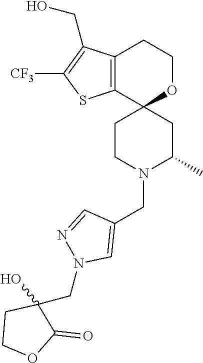 [SMILES: C[C@H]1C[C@@]2(CCN1Cc1cnn(CC3(O)CCOC3=O)c1)OCCc1c2sc(C(F)(F)F)c1CO] | 951 |
| 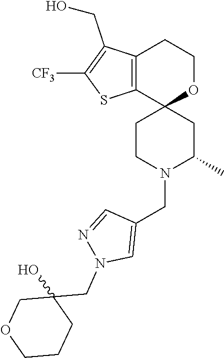 [SMILES: C[C@H]1C[C@@]2(CCN1Cc1cnn(CC3(O)CCCOC3)c1)OCCc1c2sc(C(F)(F)F)c1CO] | 952 |
| 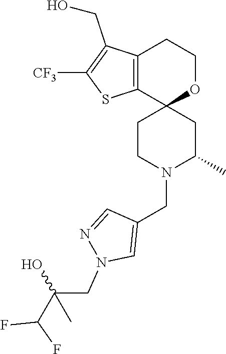 [SMILES: C[C@H]1C[C@@]2(CCN1Cc1cnn(CC(C)(O)C(F)F)c1)OCCc1c2sc(C(F)(F)F)c1CO] | 953 |
| 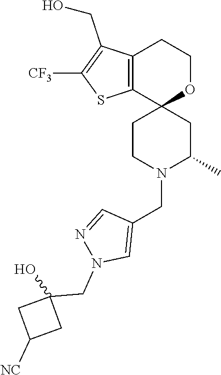 [SMILES: C[C@H]1C[C@@]2(CCN1Cc1cnn(CC3(O)CC(C#N)C3)c1)OCCc1c2sc(C(F)(F)F)c1CO] | 954 |
| 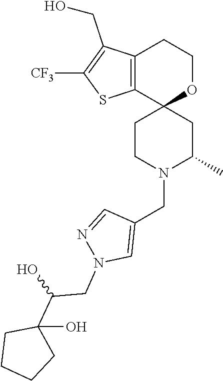 [SMILES: C[C@H]1C[C@@]2(CCN1Cc1cnn(CC(O)C3(O)CCCC3)c1)OCCc1c2sc(C(F)(F)F)c1CO] | 955 |
| 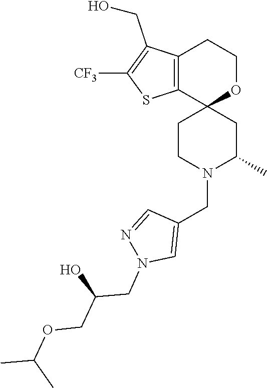 [SMILES: CC(C)OC[C@@H](O)Cn1cc(CN2CC[C@]3(C[C@@H]2C)OCCc2c3sc(C(F)(F)F)c2CO)cn1] | 956 |
| 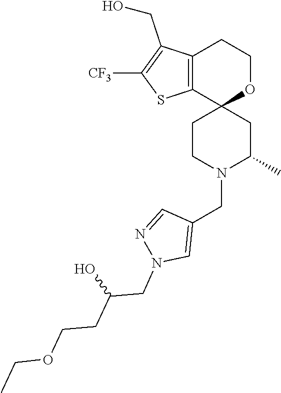 [SMILES: CCOCCC(O)Cn1cc(CN2CC[C@]3(C[C@@H]2C)OCCc2c3sc(C(F)(F)F)c2CO)cn1] | 957 |
| 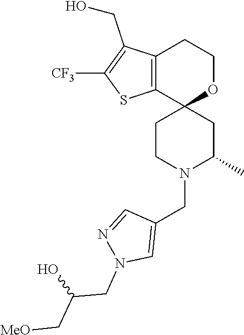 [SMILES: COCC(O)Cn1cc(CN2CC[C@]3(C[C@@H]2C)OCCc2c3sc(C(F)(F)F)c2CO)cn1] | 958 |
| 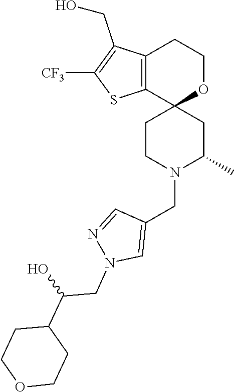 [SMILES: C[C@H]1C[C@@]2(CCN1Cc1cnn(CC(O)C3CCOCC3)c1)OCCc1c2sc(C(F)(F)F)c1CO] | 959 |
| 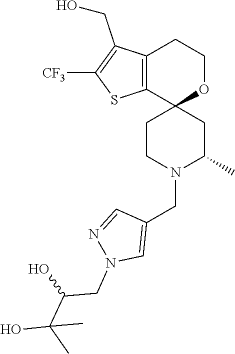 [SMILES: C[C@H]1C[C@@]2(CCN1Cc1cnn(CC(O)C(C)(C)O)c1)OCCc1c2sc(C(F)(F)F)c1CO] | 960 |
| 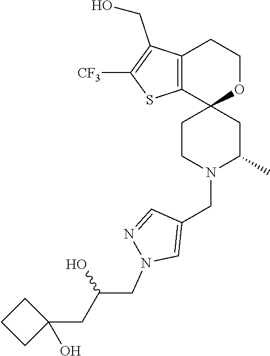 [SMILES: C[C@H]1C[C@@]2(CCN1Cc1cnn(CC(O)CC3(O)CCC3)c1)OCCc1c2sc(C(F)(F)F)c1CO] | 961 |
| 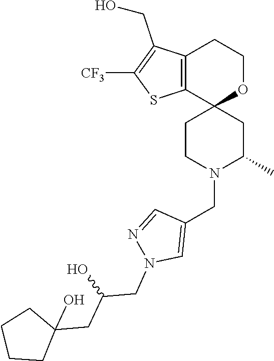 [SMILES: C[C@H]1C[C@@]2(CCN1Cc1cnn(CC(O)CC3(O)CCCC3)c1)OCCc1c2sc(C(F)(F)F)c1CO] | 962 |
| 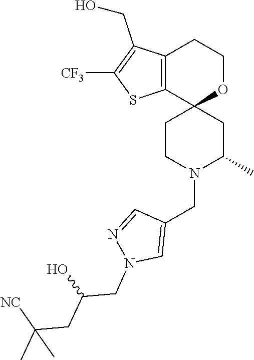 [SMILES: C[C@H]1C[C@@]2(CCN1Cc1cnn(CC(O)CC(C)(C)C#N)c1)OCCc1c2sc(C(F)(F)F)c1CO] | 963 |
| 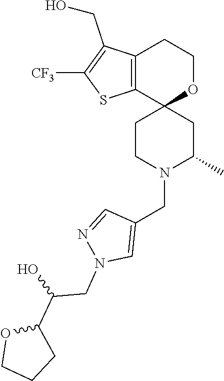 [SMILES: C[C@H]1C[C@@]2(CCN1Cc1cnn(CC(O)C3CCCO3)c1)OCCc1c2sc(C(F)(F)F)c1CO] | 964 |
| 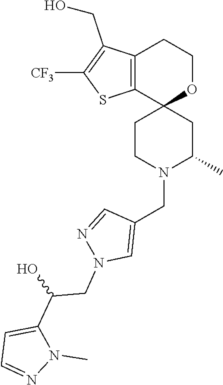 [SMILES: C[C@H]1C[C@@]2(CCN1Cc1cnn(CC(O)c3ccnn3C)c1)OCCc1c2sc(C(F)(F)F)c1CO] | 965 |
| 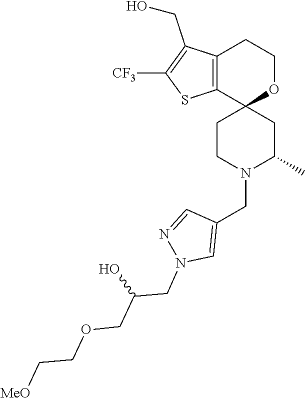 [SMILES: COCCOCC(O)Cn1cc(CN2CC[C@]3(C[C@@H]2C)OCCc2c3sc(C(F)(F)F)c2CO)cn1] | 966 |
| 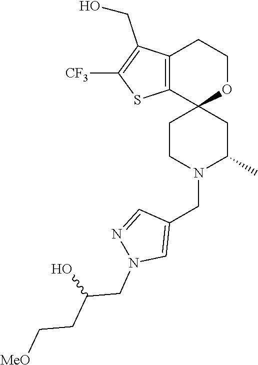 [SMILES: COCCC(O)Cn1cc(CN2CC[C@]3(C[C@@H]2C)OCCc2c3sc(C(F)(F)F)c2CO)cn1] | 967 |
| 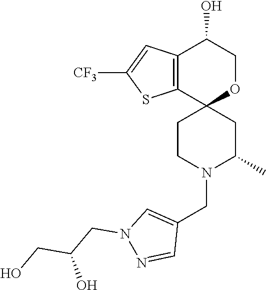 [SMILES: C[C@H]1C[C@@]2(CCN1Cc1cnn(C[C@H](O)CO)c1)OC[C@@H](O)c1cc(C(F)(F)F)sc12] | 968 |
| 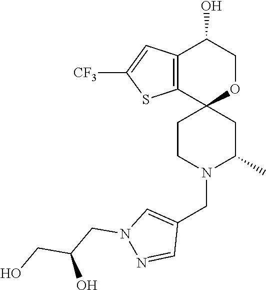 [SMILES: C[C@H]1C[C@@]2(CCN1Cc1cnn(C[C@@H](O)CO)c1)OC[C@@H](O)c1cc(C(F)(F)F)sc12] | 969 |
| 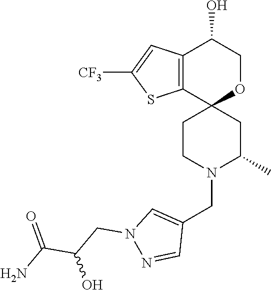 [SMILES: C[C@H]1C[C@@]2(CCN1Cc1cnn(CC(O)C(N)=O)c1)OC[C@@H](O)c1cc(C(F)(F)F)sc12] | 970 |
| 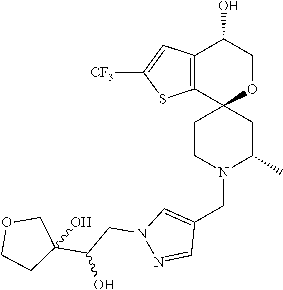 [SMILES: C[C@H]1C[C@@]2(CCN1Cc1cnn(CC(O)C3(O)CCOC3)c1)OC[C@@H](O)c1cc(C(F)(F)F)sc12] | 971 |
| 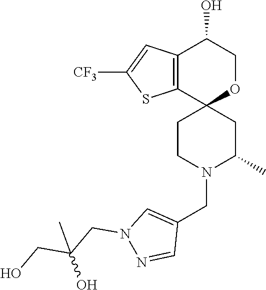 [SMILES: C[C@H]1C[C@@]2(CCN1Cc1cnn(CC(C)(O)CO)c1)OC[C@@H](O)c1cc(C(F)(F)F)sc12] | 972 |
| 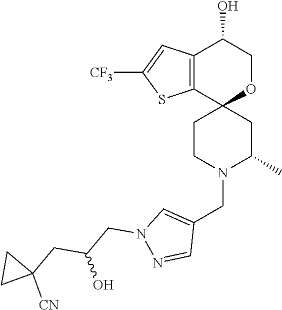 [SMILES: C[C@H]1C[C@@]2(CCN1Cc1cnn(CC(O)CC3(C#N)CC3)c1)OC[C@@H](O)c1cc(C(F)(F)F)sc12] | 973 |
| 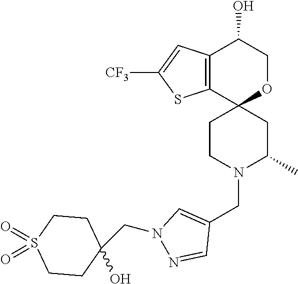 [SMILES: C[C@H]1C[C@@]2(CCN1Cc1cnn(CC3(O)CCS(=O)(=O)CC3)c1)OC[C@@H](O)c1cc(C(F)(F)F)sc12] | 974 |
| 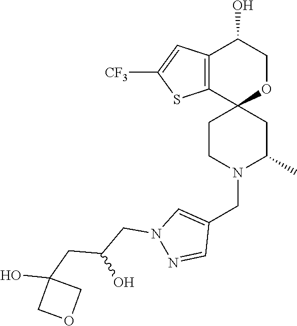 [SMILES: C[C@H]1C[C@@]2(CCN1Cc1cnn(CC(O)CC3(O)COC3)c1)OC[C@@H](O)c1cc(C(F)(F)F)sc12] | 975 |
| 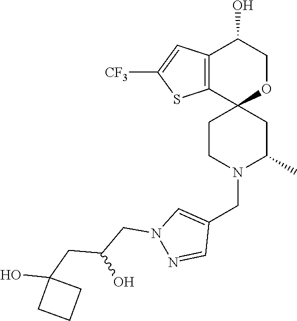 [SMILES: C[C@H]1C[C@@]2(CCN1Cc1cnn(CC(O)CC3(O)CCC3)c1)OC[C@@H](O)c1cc(C(F)(F)F)sc12] | 976 |
| 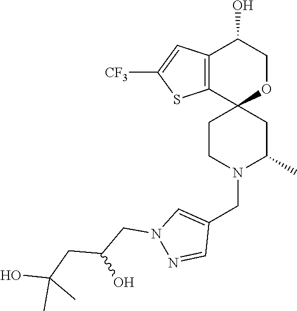 [SMILES: C[C@H]1C[C@@]2(CCN1Cc1cnn(CC(O)CC(C)(C)O)c1)OC[C@@H](O)c1cc(C(F)(F)F)sc12] | 977 |
| 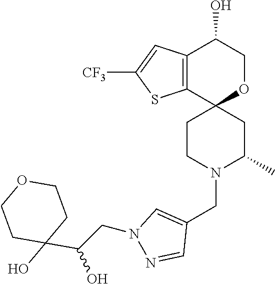 [SMILES: C[C@H]1C[C@@]2(CCN1Cc1cnn(CC(O)C3(O)CCOCC3)c1)OC[C@@H](O)c1cc(C(F)(F)F)sc12] | 978 |
| 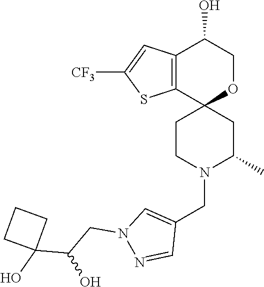 [SMILES: C[C@H]1C[C@@]2(CCN1Cc1cnn(CC(O)C3(O)CCC3)c1)OC[C@@H](O)c1cc(C(F)(F)F)sc12] | 979 |
| 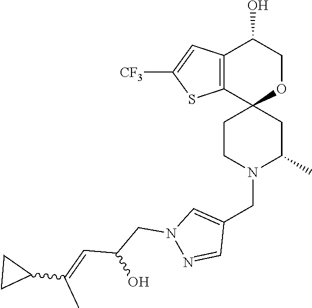 [SMILES: CC(=CC(O)Cn1cc(CN2CC[C@]3(C[C@@H]2C)OC[C@@H](O)c2cc(C(F)(F)F)sc23)cn1)C1CC1] | 980 |
| 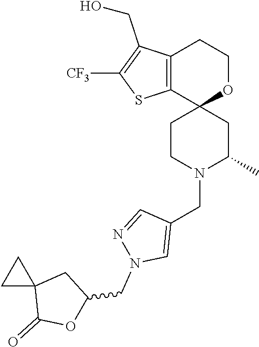 [SMILES: C[C@H]1C[C@@]2(CCN1Cc1cnn(CC3CC4(CC4)C(=O)O3)c1)OCCc1c2sc(C(F)(F)F)c1CO] | 981 |
| 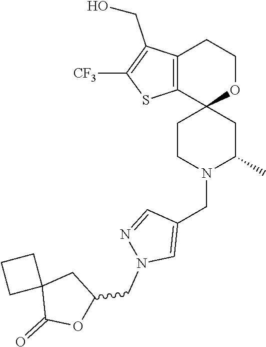 [SMILES: C[C@H]1C[C@@]2(CCN1Cc1cnn(CC3CC4(CCC4)C(=O)O3)c1)OCCc1c2sc(C(F)(F)F)c1CO] | 982 |
| 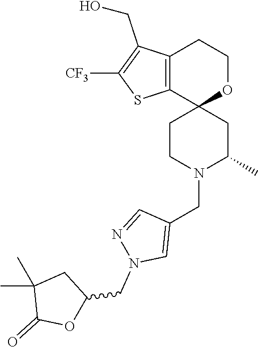 | 983 |
| 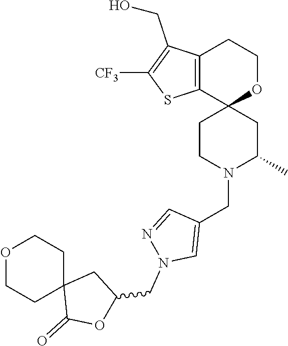 [SMILES: C[C@H]1C[C@@]2(CCN1Cc1cnn(CC3CC4(CCOCC4)C(=O)O3)c1)OCCc1c2sc(C(F)(F)F)c1CO] | 984 |
| 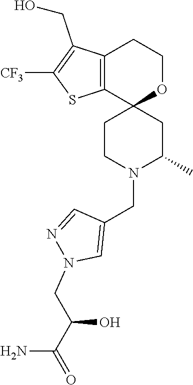 [SMILES: C[C@H]1C[C@@]2(CCN1Cc1cnn(C[C@@H](O)C(N)=O)c1)OCCc1c2sc(C(F)(F)F)c1CO] | 985 |
| 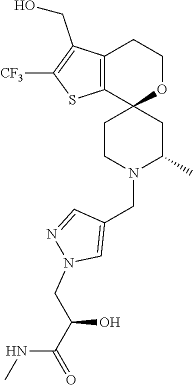 | 986 |
| 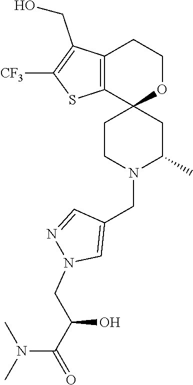 | 987 |
| 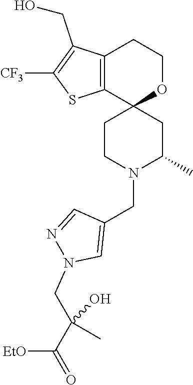 [SMILES: CCOC(=O)C(C)(O)Cn1cc(CN2CC[C@]3(C[C@@H]2C)OCCc2c3sc(C(F)(F)F)c2CO)cn1] | 988 |
| 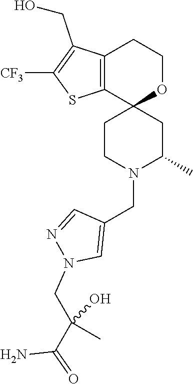 [SMILES: C[C@H]1C[C@@]2(CCN1Cc1cnn(CC(C)(O)C(N)=O)c1)OCCc1c2sc(C(F)(F)F)c1CO] | 989 |
| 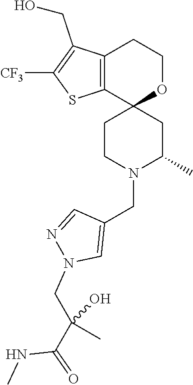 | 990 |
| 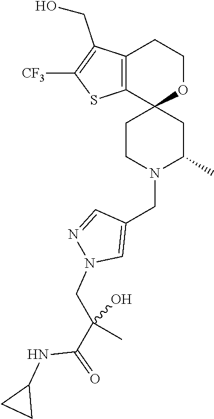 [SMILES: C[C@H]1C[C@@]2(CCN1Cc1cnn(CC(C)(O)C(=O)NC3CC3)c1)OCCc1c2sc(C(F)(F)F)c1CO] | 991 |
| 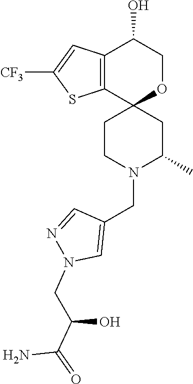 [SMILES: C[C@H]1C[C@@]2(CCN1Cc1cnn(C[C@@H](O)C(N)=O)c1)OC[C@@H](O)c1cc(C(F)(F)F)sc12] | 992 |
| 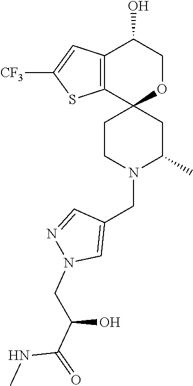 | 993 |
| 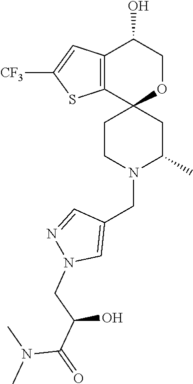 | 994 |
| 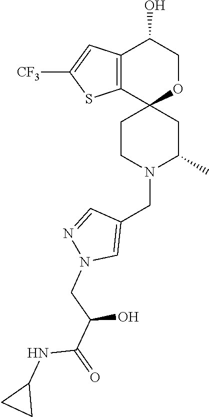 [SMILES: C[C@H]1C[C@@]2(CCN1Cc1cnn(C[C@@H](O)C(=O)NC3CC3)c1)OC[C@@H](O)c1cc(C(F)(F)F)sc12] | 995 |
| 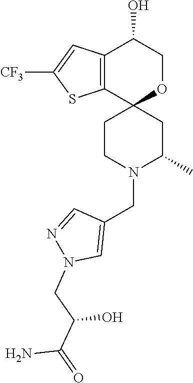 [SMILES: C[C@H]1C[C@@]2(CCN1Cc1cnn(C[C@H](O)C(N)=O)c1)OC[C@@H](O)c1cc(C(F)(F)F)sc12] | 996 |
| 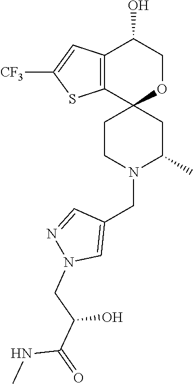 | 997 |
| 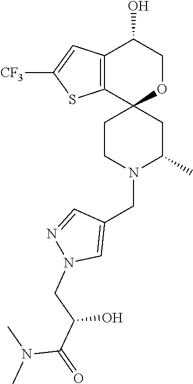 | 998 |
| 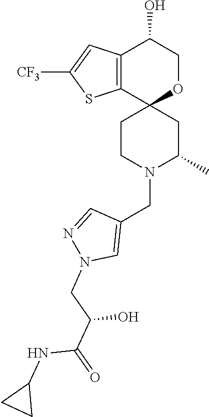 [SMILES: C[C@H]1C[C@@]2(CCN1Cc1cnn(C[C@H](O)C(=O)NC3CC3)c1)OC[C@@H](O)c1cc(C(F)(F)F)sc12] | 999 |
| 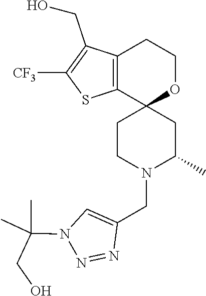 [SMILES: C[C@H]1C[C@@]2(CCN1Cc1cn(C(C)(C)CO)nn1)OCCc1c2sc(C(F)(F)F)c1CO] | 1000 |
| 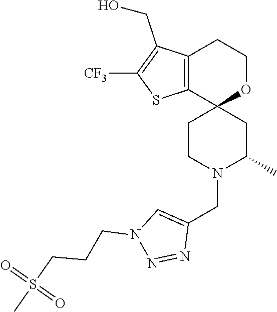 [SMILES: C[C@H]1C[C@@]2(CCN1Cc1cn(CCCS(C)(=O)=O)nn1)OCCc1c2sc(C(F)(F)F)c1CO] | 1001 |
| 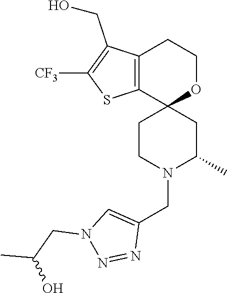 [SMILES: CC(O)Cn1cc(CN2CC[C@]3(C[C@@H]2C)OCCc2c3sc(C(F)(F)F)c2CO)nn1] | 1002 |
| 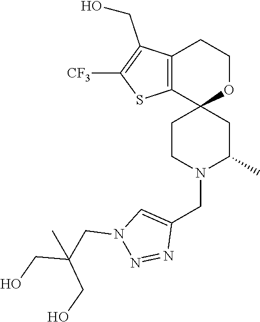 [SMILES: C[C@H]1C[C@@]2(CCN1Cc1cn(CC(C)(CO)CO)nn1)OCCc1c2sc(C(F)(F)F)c1CO] | 1003 |
| 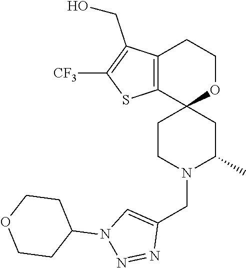 [SMILES: C[C@H]1C[C@@]2(CCN1Cc1cn(C3CCOCC3)nn1)OCCc1c2sc(C(F)(F)F)c1CO] | 1004 |
| 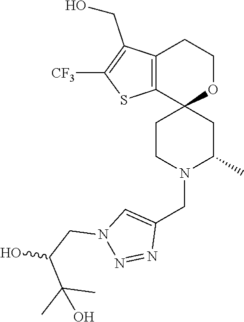 [SMILES: C[C@H]1C[C@@]2(CCN1Cc1cn(CC(O)C(C)(C)O)nn1)OCCc1c2sc(C(F)(F)F)c1CO] | 1005 |
| 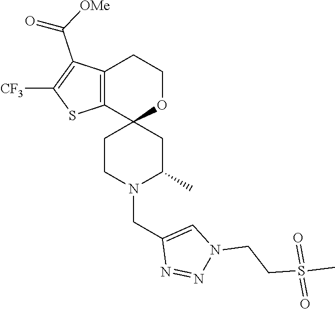 [SMILES: COC(=O)c1c(C(F)(F)F)sc2c1CCO[C@@]21CCN(Cc2cn(CCS(C)(=O)=O)nn2)[C@@H](C)C1] | 1006 |
| 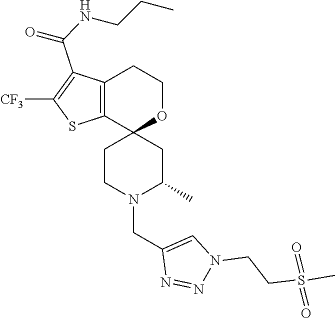 [SMILES: CCCNC(=O)c1c(C(F)(F)F)sc2c1CCO[C@@]21CCN(Cc2cn(CCS(C)(=O)=O)nn2)[C@@H](C)C1] | 1007 |
| 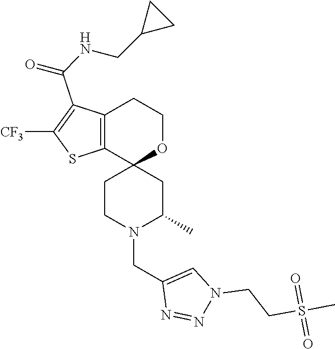 [SMILES: C[C@H]1C[C@@]2(CCN1Cc1cn(CCS(C)(=O)=O)nn1)OCCc1c2sc(C(F)(F)F)c1C(=O)NCC1CC1] | 1008 |
| 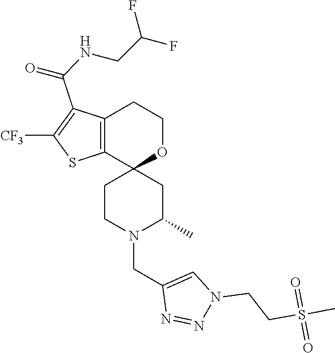 [SMILES: C[C@H]1C[C@@]2(CCN1Cc1cn(CCS(C)(=O)=O)nn1)OCCc1c2sc(C(F)(F)F)c1C(=O)NCC(F)F] | 1009 |
| 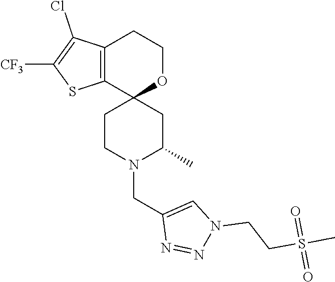 [SMILES: C[C@H]1C[C@@]2(CCN1Cc1cn(CCS(C)(=O)=O)nn1)OCCc1c2sc(C(F)(F)F)c1Cl] | 1010 |
| 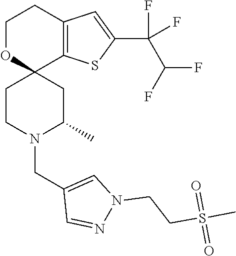 [SMILES: C[C@H]1C[C@@]2(CCN1Cc1cnn(CCS(C)(=O)=O)c1)OCCc1cc(C(F)(F)C(F)F)sc12] | 1011 |
| 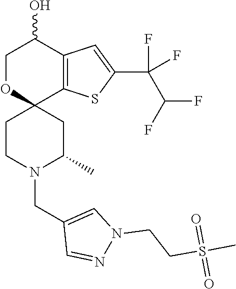 | 1012 |
| 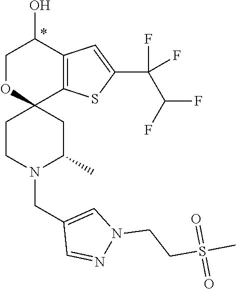 | 1013 |
| 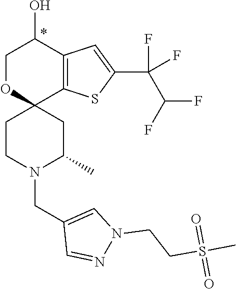 | 1014 |
| 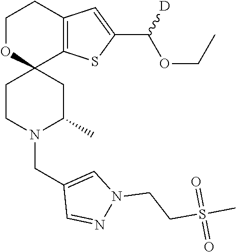 [SMILES: [2H]C(OCC)c1cc2c(s1)[C@]1(CCN(Cc3cnn(CCS(C)(=O)=O)c3)[C@@H](C)C1)OCC2] | 1015 |
| 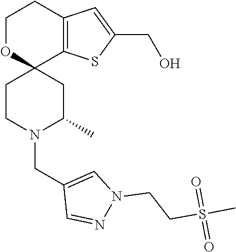 [SMILES: C[C@H]1C[C@@]2(CCN1Cc1cnn(CCS(C)(=O)=O)c1)OCCc1cc(CO)sc12] | 1016 |
| 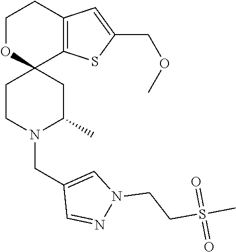 [SMILES: COCc1cc2c(s1)[C@]1(CCN(Cc3cnn(CCS(C)(=O)=O)c3)[C@@H](C)C1)OCC2] | 1017 |
| 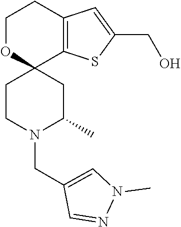 [SMILES: C[C@H]1C[C@@]2(CCN1Cc1cnn(C)c1)OCCc1cc(CO)sc12] | 1018 |
| 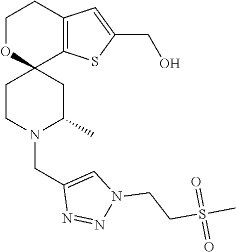 [SMILES: C[C@H]1C[C@@]2(CCN1Cc1cn(CCS(C)(=O)=O)nn1)OCCc1cc(CO)sc12] | 1019 |
| 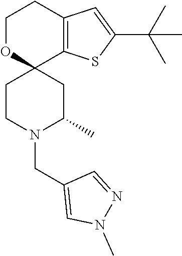 [SMILES: C[C@H]1C[C@@]2(CCN1Cc1cnn(C)c1)OCCc1cc(C(C)(C)C)sc12] | 1020 |
| 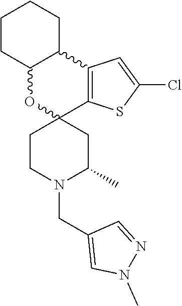 | 1021 |
| 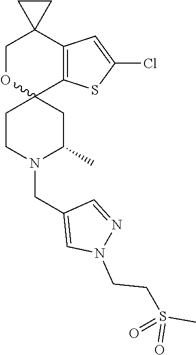 [SMILES: C[C@H]1CC2(CCN1Cc1cnn(CCS(C)(=O)=O)c1)OCC1(CC1)c1cc(Cl)sc12] | 1022 |
| 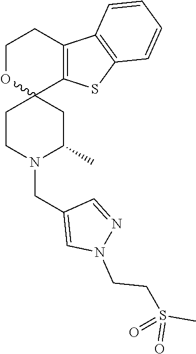 [SMILES: C[C@H]1CC2(CCN1Cc1cnn(CCS(C)(=O)=O)c1)OCCc1c2sc2ccccc12] | 1023 |
| 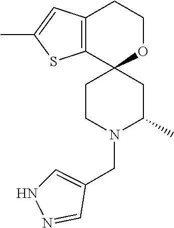 | 1024 |
| 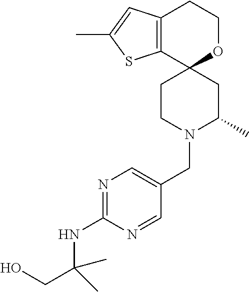 [SMILES: Cc1cc2c(s1)[C@]1(CCN(Cc3cnc(NC(C)(C)CO)nc3)[C@@H](C)C1)OCC2] | 1025 |
| 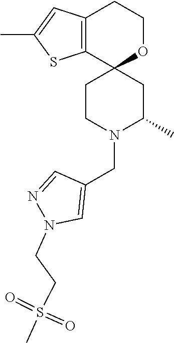 | 1026 |
| 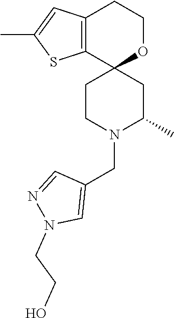 | 1027 |
| 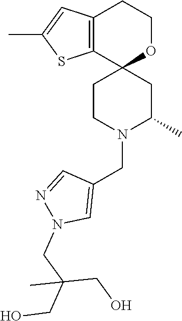 | 1028 |
| 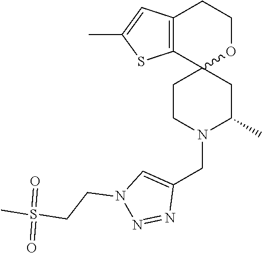 | 1029 |
| 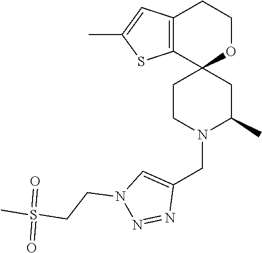 | 1030 |
| 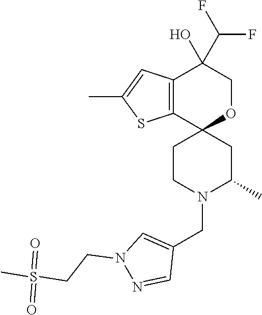 | 1031 |
| 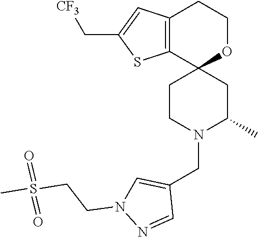 | 1032 |
| 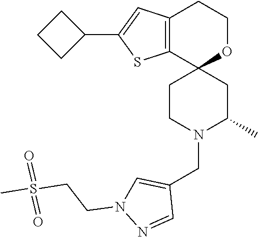 | 1033 |
| 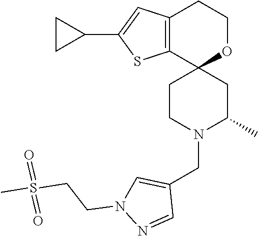 | 1034 |
| 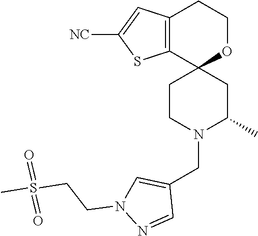 | 1035 |
| 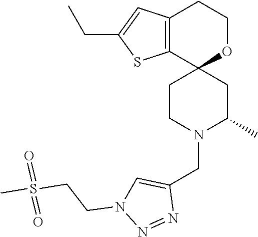 | 1036 |
| 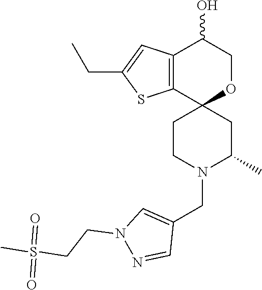 | 1037 |
| 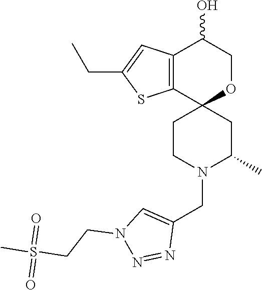 | 1038 |
| 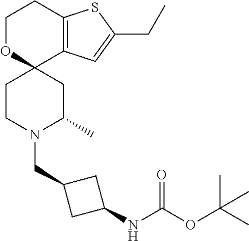 [SMILES: CCc1cc2c(s1)CCO[C@@]21CCN(C[C@H]2C[C@@H](NC(=O)OC(C)(C)C)C2)[C@@H](C)C1] | 1039 |
| 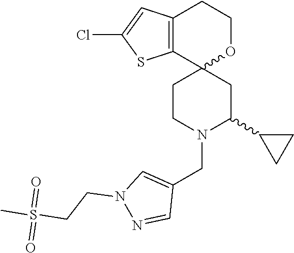 [SMILES: CS(=O)(=O)CCn1cc(CN2CCC3(CC2C2CC2)OCCc2cc(Cl)sc23)cn1] | 1040 |
| 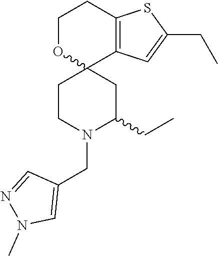 [SMILES: CCc1cc2c(s1)CCOC21CCN(Cc2cnn(C)c2)C(CC)C1] | 1041 |
| 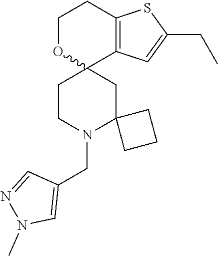 [SMILES: CCc1cc2c(s1)CCOC21CCN(Cc2cnn(C)c2)C2(CCC2)C1] | 1042 |
| 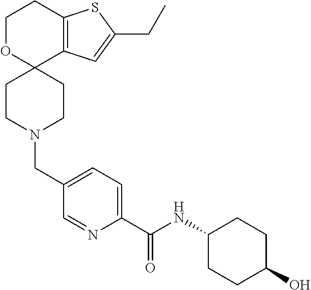 [SMILES: CCc1cc2c(s1)CCOC21CCN(Cc2ccc(C(=O)N[C@H]3CC[C@H](O)CC3)nc2)CC1] | 1043 |
| 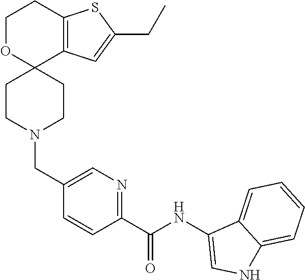 [SMILES: CCc1cc2c(s1)CCOC21CCN(Cc2ccc(C(=O)Nc3c[nH]c4ccccc34)nc2)CC1] | 1044 |
| 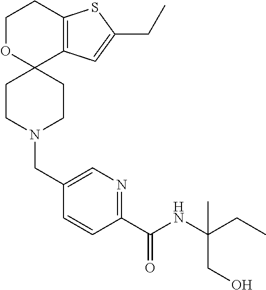 [SMILES: CCc1cc2c(s1)CCOC21CCN(Cc2ccc(C(=O)NC(C)(CC)CO)nc2)CC1] | 1045 |
| 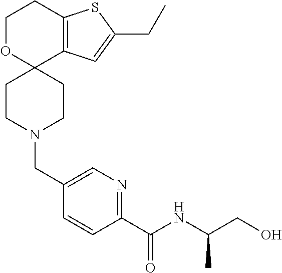 [SMILES: CCc1cc2c(s1)CCOC21CCN(Cc2ccc(C(=O)N[C@H](C)CO)nc2)CC1] | 1046 |
| 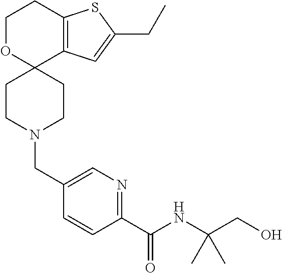 [SMILES: CCc1cc2c(s1)CCOC21CCN(Cc2ccc(C(=O)NC(C)(C)CO)nc2)CC1] | 1047 |
| 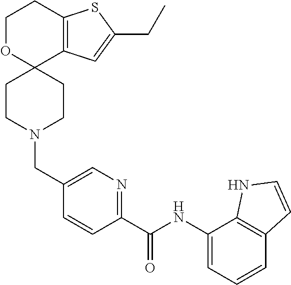 [SMILES: CCc1cc2c(s1)CCOC21CCN(Cc2ccc(C(=O)Nc3cccc4cc[nH]c34)nc2)CC1] | 1048 |
| 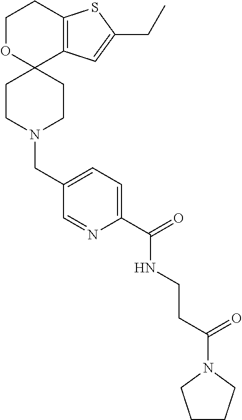 [SMILES: CCc1cc2c(s1)CCOC21CCN(Cc2ccc(C(=O)NCCC(=O)N3CCCC3)nc2)CC1] | 1049 |
| 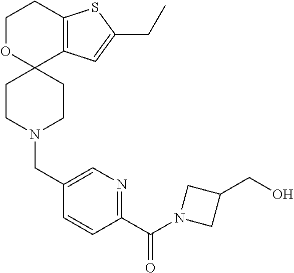 [SMILES: CCc1cc2c(s1)CCOC21CCN(Cc2ccc(C(=O)N3CC(CO)C3)nc2)CC1] | 1050 |
| 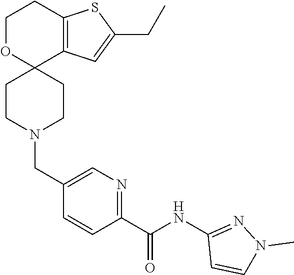 [SMILES: CCc1cc2c(s1)CCOC21CCN(Cc2ccc(C(=O)Nc3ccn(C)n3)nc2)CC1] | 1051 |
| 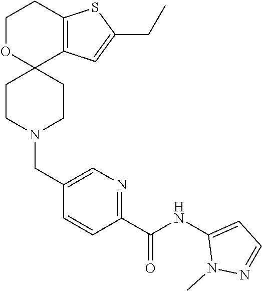 [SMILES: CCc1cc2c(s1)CCOC21CCN(Cc2ccc(C(=O)Nc3ccnn3C)nc2)CC1] | 1052 |
| 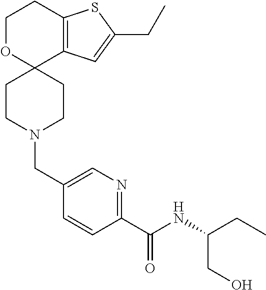 [SMILES: CCc1cc2c(s1)CCOC21CCN(Cc2ccc(C(=O)N[C@H](CC)CO)nc2)CC1] | 1053 |
| 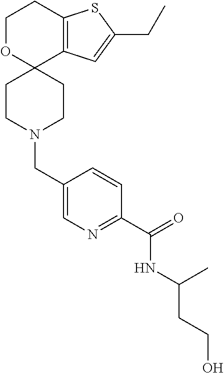 [SMILES: CCc1cc2c(s1)CCOC21CCN(Cc2ccc(C(=O)NC(C)CCO)nc2)CC1] | 1054 |
| 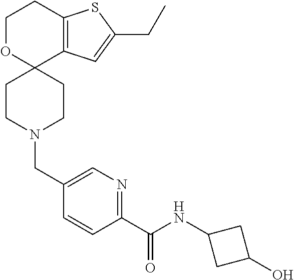 [SMILES: CCc1cc2c(s1)CCOC21CCN(Cc2ccc(C(=O)NC3CC(O)C3)nc2)CC1] | 1055 |
| 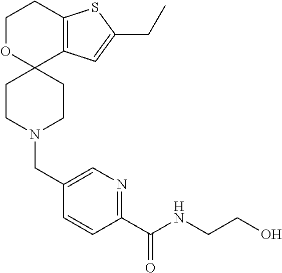 [SMILES: CCc1cc2c(s1)CCOC21CCN(Cc2ccc(C(=O)NCCO)nc2)CC1] | 1056 |
| 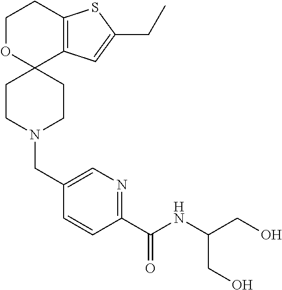 [SMILES: CCc1cc2c(s1)CCOC21CCN(Cc2ccc(C(=O)NC(CO)CO)nc2)CC1] | 1057 |
| 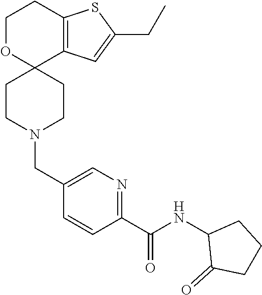 [SMILES: CCc1cc2c(s1)CCOC21CCN(Cc2ccc(C(=O)NC3CCCC3=O)nc2)CC1] | 1058 |
| 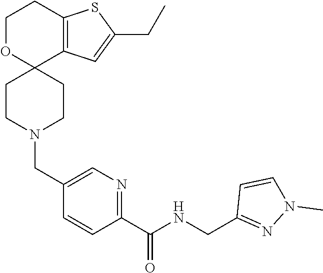 [SMILES: CCc1cc2c(s1)CCOC21CCN(Cc2ccc(C(=O)NCc3ccn(C)n3)nc2)CC1] | 1059 |
| 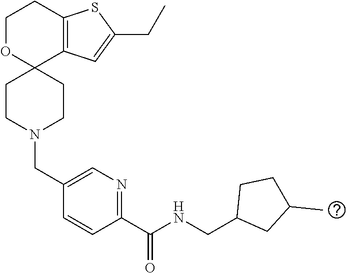 [SMILES: CCc1cc2c(s1)CCOC21CCN(Cc2ccc(C(=O)NCC3CCC(C)C3)nc2)CC1] | 1060 |
| 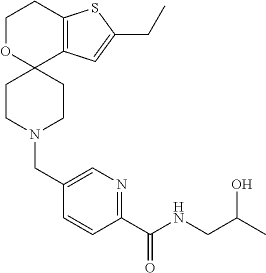 [SMILES: CCc1cc2c(s1)CCOC21CCN(Cc2ccc(C(=O)NCC(C)O)nc2)CC1] | 1061 |
| 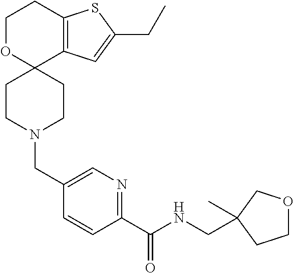 [SMILES: CCc1cc2c(s1)CCOC21CCN(Cc2ccc(C(=O)NCC3(C)CCOC3)nc2)CC1] | 1062 |
| 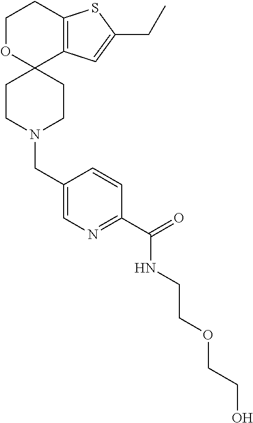 [SMILES: CCc1cc2c(s1)CCOC21CCN(Cc2ccc(C(=O)NCCOCCO)nc2)CC1] | 1063 |
| 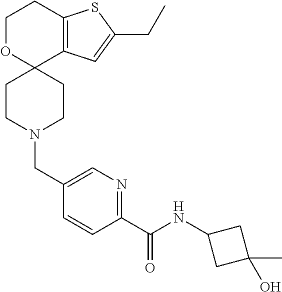 [SMILES: CCc1cc2c(s1)CCOC21CCN(Cc2ccc(C(=O)NC3CC(C)(O)C3)nc2)CC1] | 1064 |
| 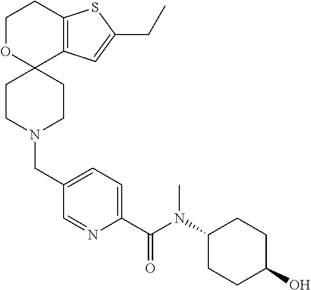 [SMILES: CCc1cc2c(s1)CCOC21CCN(Cc2ccc(C(=O)N(C)[C@H]3CC[C@H](O)CC3)nc2)CC1] | 1065 |
| 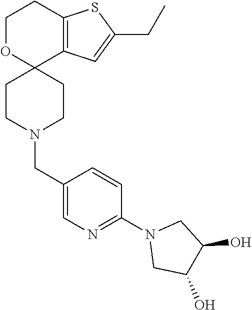 [SMILES: CCc1cc2c(s1)CCOC21CCN(Cc2ccc(N3C[C@@H](O)[C@H](O)C3)nc2)CC1] | 1066 |
| 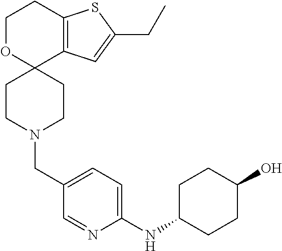 [SMILES: CCc1cc2c(s1)CCOC21CCN(Cc2ccc(N[C@H]3CC[C@H](O)CC3)nc2)CC1] | 1067 |
| 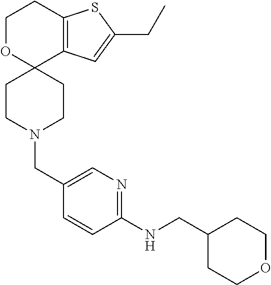 [SMILES: CCc1cc2c(s1)CCOC21CCN(Cc2ccc(NCC3CCOCC3)nc2)CC1] | 1068 |
| 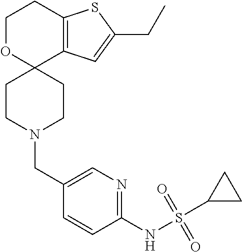 [SMILES: CCc1cc2c(s1)CCOC21CCN(Cc2ccc(NS(=O)(=O)C3CC3)nc2)CC1] | 1069 |
| 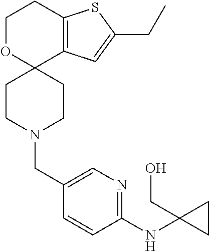 [SMILES: CCc1cc2c(s1)CCOC21CCN(Cc2ccc(NC3(CO)CC3)nc2)CC1] | 1070 |
| 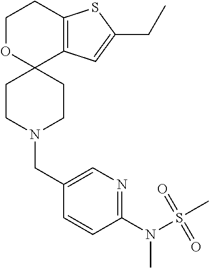 [SMILES: CCc1cc2c(s1)CCOC21CCN(Cc2ccc(N(C)S(C)(=O)=O)nc2)CC1] | 1071 |
| 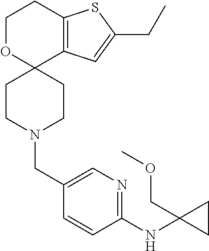 [SMILES: CCc1cc2c(s1)CCOC21CCN(Cc2ccc(NC3(COC)CC3)nc2)CC1] | 1072 |
| 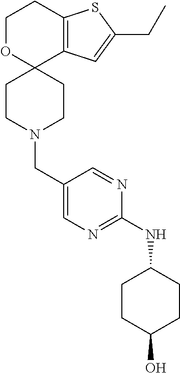 [SMILES: CCc1cc2c(s1)CCOC21CCN(Cc2cnc(N[C@H]3CC[C@H](O)CC3)nc2)CC1] | 1073 |
| 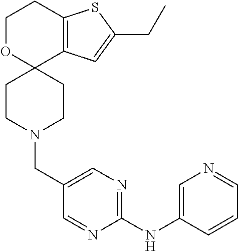 [SMILES: CCc1cc2c(s1)CCOC21CCN(Cc2cnc(Nc3cccnc3)nc2)CC1] | 1074 |
| 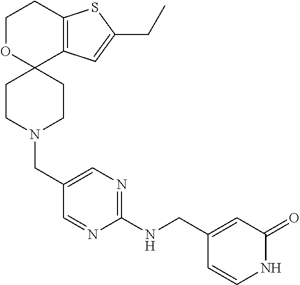 [SMILES: CCc1cc2c(s1)CCOC21CCN(Cc2cnc(NCc3cc[nH]c(=O)c3)nc2)CC1] | 1075 |
| 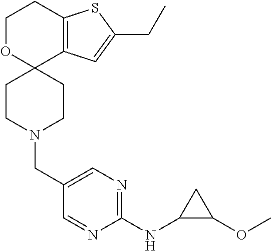 [SMILES: CCc1cc2c(s1)CCOC21CCN(Cc2cnc(NC3CC3OC)nc2)CC1] | 1076 |
| 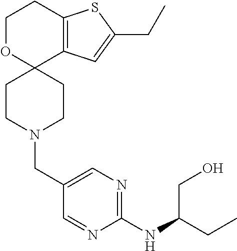 [SMILES: CCc1cc2c(s1)CCOC21CCN(Cc2cnc(N[C@H](CC)CO)nc2)CC1] | 1077 |
| 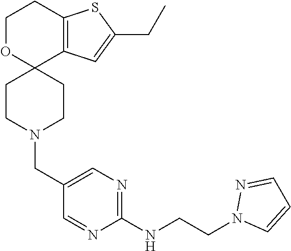 [SMILES: CCc1cc2c(s1)CCOC21CCN(Cc2cnc(NCCn3cccn3)nc2)CC1] | 1078 |
| 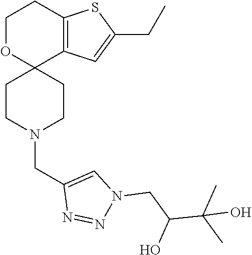 [SMILES: CCc1cc2c(s1)CCOC21CCN(Cc2cn(CC(O)C(C)(C)O)nn2)CC1] | 1079 |
| 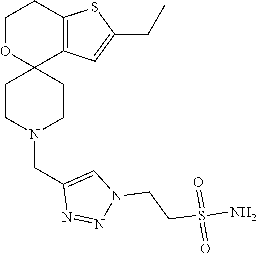 [SMILES: CCc1cc2c(s1)CCOC21CCN(Cc2cn(CCS(N)(=O)=O)nn2)CC1] | 1080 |
| 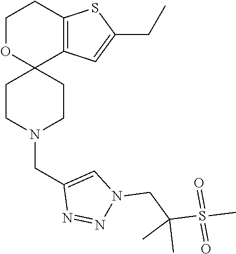 [SMILES: CCc1cc2c(s1)CCOC21CCN(Cc2cn(CC(C)(C)S(C)(=O)=O)nn2)CC1] | 1081 |
| 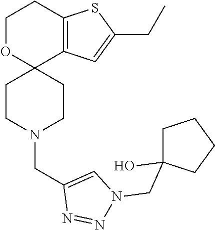 [SMILES: CCc1cc2c(s1)CCOC21CCN(Cc2cn(CC3(O)CCCC3)nn2)CC1] | 1082 |
| 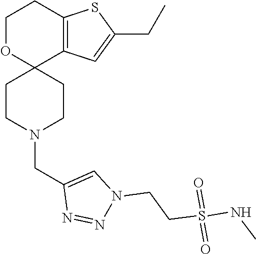 [SMILES: CCc1cc2c(s1)CCOC21CCN(Cc2cn(CCS(=O)(=O)NC)nn2)CC1] | 1083 |
| 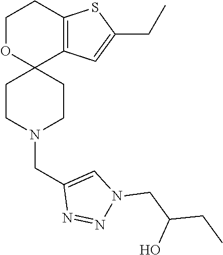 [SMILES: CCc1cc2c(s1)CCOC21CCN(Cc2cn(CC(O)CC)nn2)CC1] | 1084 |
| 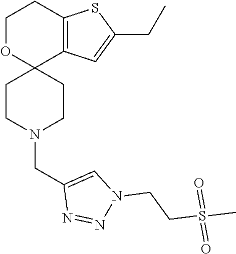 [SMILES: CCc1cc2c(s1)CCOC21CCN(Cc2cn(CCS(C)(=O)=O)nn2)CC1] | 1085 |
| 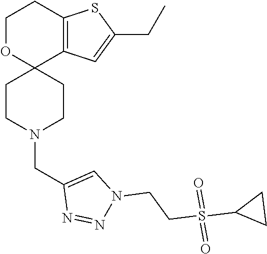 [SMILES: CCc1cc2c(s1)CCOC21CCN(Cc2cn(CCS(=O)(=O)C3CC3)nn2)CC1] | 1086 |
| 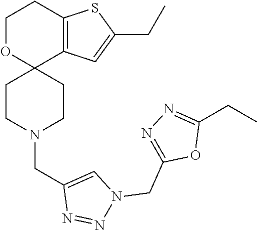 [SMILES: CCc1nnc(Cn2cc(CN3CCC4(CC3)OCCc3sc(CC)cc34)nn2)o1] | 1087 |
| 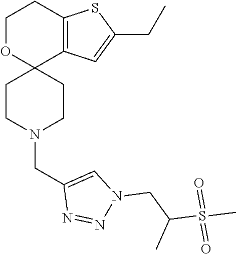 [SMILES: CCc1cc2c(s1)CCOC21CCN(Cc2cn(CC(C)S(C)(=O)=O)nn2)CC1] | 1088 |
| 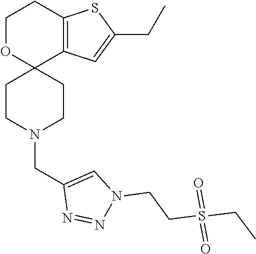 [SMILES: CCc1cc2c(s1)CCOC21CCN(Cc2cn(CCS(=O)(=O)CC)nn2)CC1] | 1089 |
| 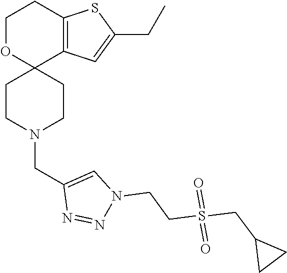 [SMILES: CCc1cc2c(s1)CCOC21CCN(Cc2cn(CCS(=O)(=O)CC3CC3)nn2)CC1] | 1090 |
| 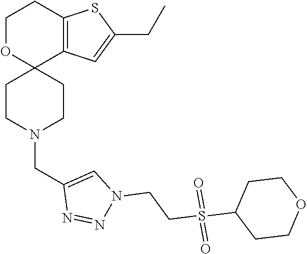 [SMILES: CCc1cc2c(s1)CCOC21CCN(Cc2cn(CCS(=O)(=O)C3CCOCC3)nn2)CC1] | 1091 |
| 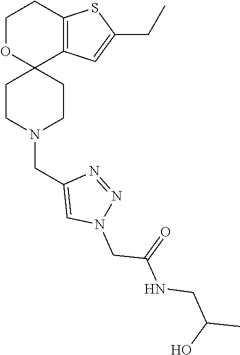 [SMILES: CCc1cc2c(s1)CCOC21CCN(Cc2cn(CC(=O)NCC(C)O)nn2)CC1] | 1092 |
| 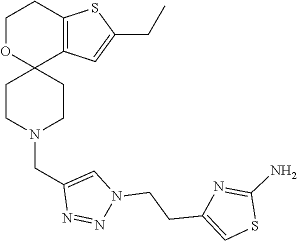 [SMILES: CCc1cc2c(s1)CCOC21CCN(Cc2cn(CCc3csc(N)n3)nn2)CC1] | 1093 |
| 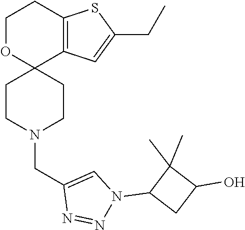 [SMILES: CCc1cc2c(s1)CCOC21CCN(Cc2cn(C3CC(O)C3(C)C)nn2)CC1] | 1094 |
| 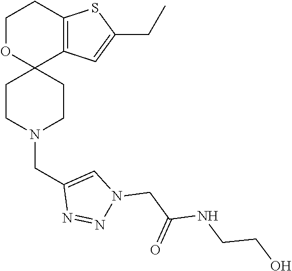 [SMILES: CCc1cc2c(s1)CCOC21CCN(Cc2cn(CC(=O)NCCO)nn2)CC1] | 1095 |
| 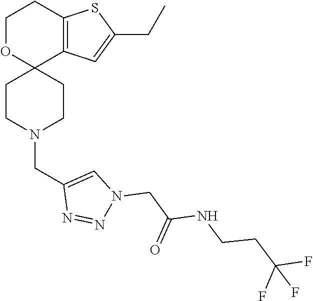 [SMILES: CCc1cc2c(s1)CCOC21CCN(Cc2cn(CC(=O)NCCC(F)(F)F)nn2)CC1] | 1096 |
| 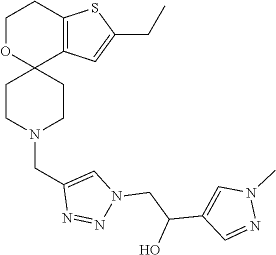 [SMILES: CCc1cc2c(s1)CCOC21CCN(Cc2cn(CC(O)c3cnn(C)c3)nn2)CC1] | 1097 |
| 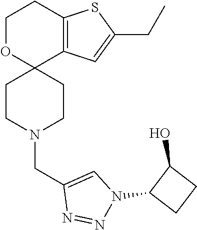 [SMILES: CCc1cc2c(s1)CCOC21CCN(Cc2cn([C@H]3CC[C@@H]3O)nn2)CC1] | 1098 |
| 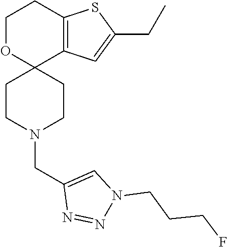 [SMILES: CCc1cc2c(s1)CCOC21CCN(Cc2cn(CCCF)nn2)CC1] | 1099 |
| 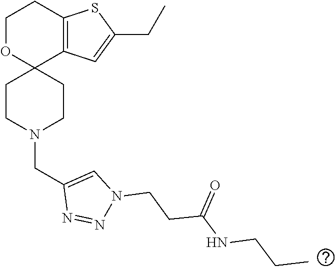 [SMILES: CCCNC(=O)CCn1cc(CN2CCC3(CC2)OCCc2sc(CC)cc23)nn1] | 1100 |
| 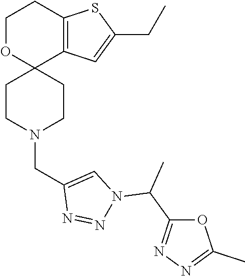 [SMILES: CCc1cc2c(s1)CCOC21CCN(Cc2cn(C(C)c3nnc(C)o3)nn2)CC1] | 1101 |
| 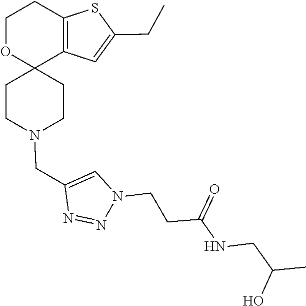 [SMILES: CCc1cc2c(s1)CCOC21CCN(Cc2cn(CCC(=O)NCC(C)O)nn2)CC1] | 1102 |
| 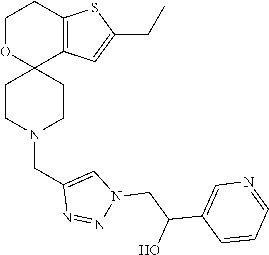 [SMILES: CCc1cc2c(s1)CCOC21CCN(Cc2cn(CC(O)c3cccnc3)nn2)CC1] | 1103 |
| 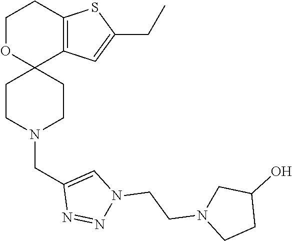 [SMILES: CCc1cc2c(s1)CCOC21CCN(Cc2cn(CCN3CCC(O)C3)nn2)CC1] | 1104 |
| 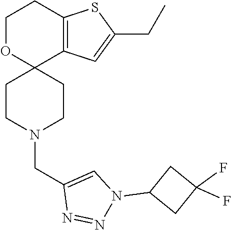 [SMILES: CCc1cc2c(s1)CCOC21CCN(Cc2cn(C3CC(F)(F)C3)nn2)CC1] | 1105 |
| 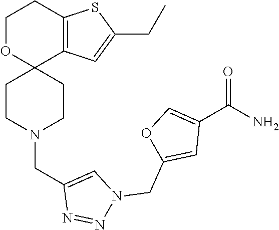 [SMILES: CCc1cc2c(s1)CCOC21CCN(Cc2cn(Cc3cc(C(N)=O)co3)nn2)CC1] | 1106 |
| 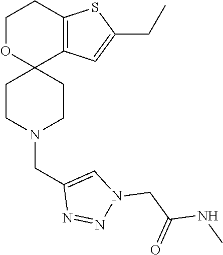 [SMILES: CCc1cc2c(s1)CCOC21CCN(Cc2cn(CC(=O)NC)nn2)CC1] | 1107 |
| 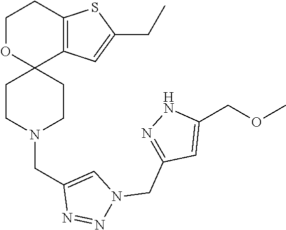 [SMILES: CCc1cc2c(s1)CCOC21CCN(Cc2cn(Cc3cc(COC)[nH]n3)nn2)CC1] | 1108 |
| 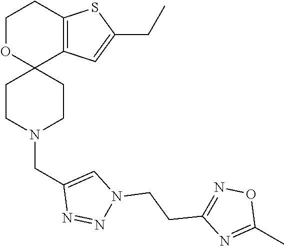 [SMILES: CCc1cc2c(s1)CCOC21CCN(Cc2cn(CCc3noc(C)n3)nn2)CC1] | 1109 |
| 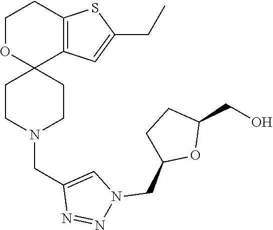 [SMILES: CCc1cc2c(s1)CCOC21CCN(Cc2cn(C[C@H]3CC[C@@H](CO)O3)nn2)CC1] | 1110 |
| 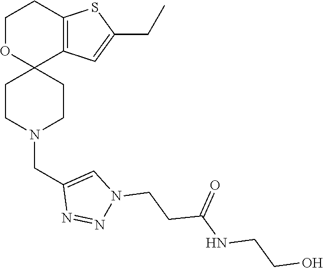 [SMILES: CCc1cc2c(s1)CCOC21CCN(Cc2cn(CCC(=O)NCCO)nn2)CC1] | 1111 |
| 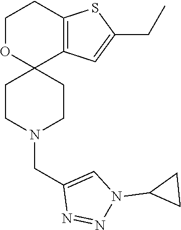 [SMILES: CCc1cc2c(s1)CCOC21CCN(Cc2cn(C3CC3)nn2)CC1] | 1112 |
| 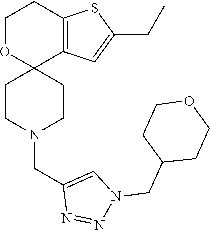 [SMILES: CCc1cc2c(s1)CCOC21CCN(Cc2cn(CC3CCOCC3)nn2)CC1] | 1113 |
| 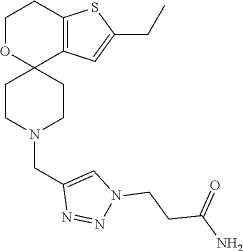 [SMILES: CCc1cc2c(s1)CCOC21CCN(Cc2cn(CCC(N)=O)nn2)CC1] | 1114 |
| 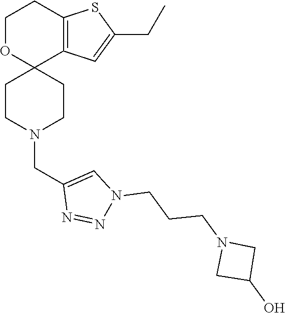 [SMILES: CCc1cc2c(s1)CCOC21CCN(Cc2cn(CCCN3CC(O)C3)nn2)CC1] | 1115 |
| 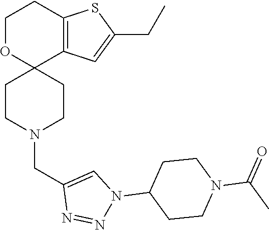 [SMILES: CCc1cc2c(s1)CCOC21CCN(Cc2cn(C3CCN(C(C)=O)CC3)nn2)CC1] | 1116 |
| 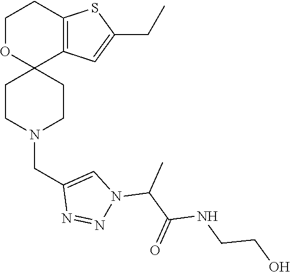 [SMILES: CCc1cc2c(s1)CCOC21CCN(Cc2cn(C(C)C(=O)NCCO)nn2)CC1] | 1117 |
| 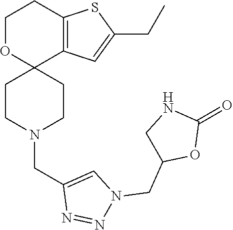 [SMILES: CCc1cc2c(s1)CCOC21CCN(Cc2cn(CC3CNC(=O)O3)nn2)CC1] | 1118 |
| 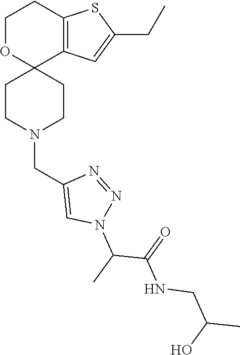 [SMILES: CCc1cc2c(s1)CCOC21CCN(Cc2cn(C(C)C(=O)NCC(C)O)nn2)CC1] | 1119 |
| 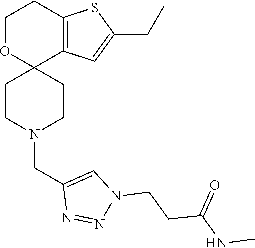 [SMILES: CCc1cc2c(s1)CCOC21CCN(Cc2cn(CCC(=O)NC)nn2)CC1] | 1120 |
| 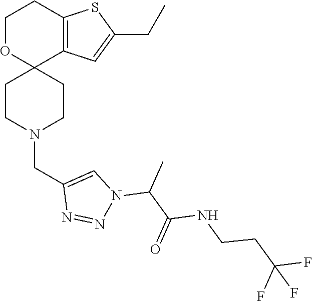 [SMILES: CCc1cc2c(s1)CCOC21CCN(Cc2cn(C(C)C(=O)NCCC(F)(F)F)nn2)CC1] | 1121 |
| 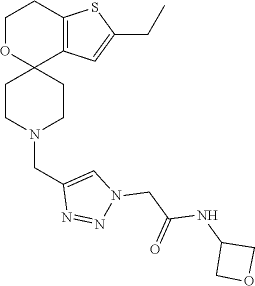 [SMILES: CCc1cc2c(s1)CCOC21CCN(Cc2cn(CC(=O)NC3COC3)nn2)CC1] | 1122 |
| 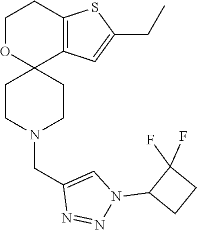 [SMILES: CCc1cc2c(s1)CCOC21CCN(Cc2cn(C3CCC3(F)F)nn2)CC1] | 1123 |
| 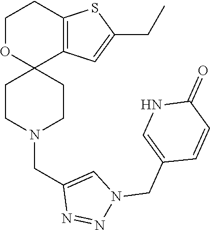 [SMILES: CCc1cc2c(s1)CCOC21CCN(Cc2cn(Cc3ccc(=O)[nH]c3)nn2)CC1] | 1124 |
| 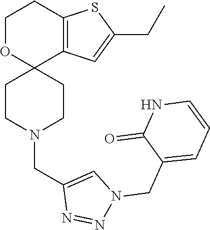 [SMILES: CCc1cc2c(s1)CCOC21CCN(Cc2cn(Cc3ccc[nH]c3=O)nn2)CC1] | 1125 |
| 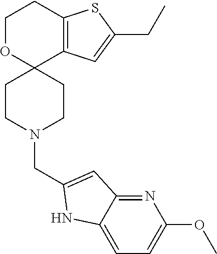 [SMILES: CCc1cc2c(s1)CCOC21CCN(Cc2cc3nc(OC)ccc3[nH]2)CC1] | 1126 |
| 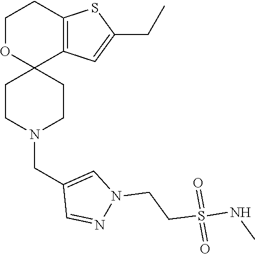 [SMILES: CCc1cc2c(s1)CCOC21CCN(Cc2cnn(CCS(=O)(=O)NC)c2)CC1] | 1127 |
| 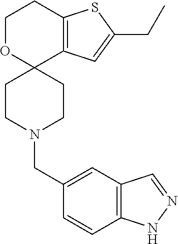 [SMILES: CCc1cc2c(s1)CCOC21CCN(Cc2ccc3[nH]ncc3c2)CC1] | 1128 |
| 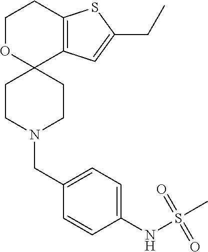 [SMILES: CCc1cc2c(s1)CCOC21CCN(Cc2ccc(NS(C)(=O)=O)cc2)CC1] | 1129 |
| 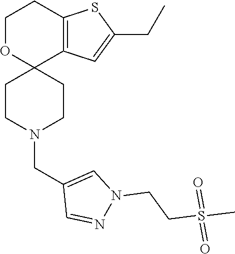 [SMILES: CCc1cc2c(s1)CCOC21CCN(Cc2cnn(CCS(C)(=O)=O)c2)CC1] | 1130 |
| 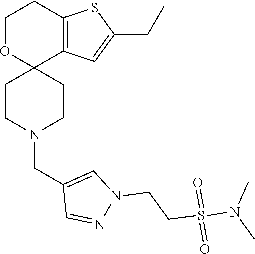 [SMILES: CCc1cc2c(s1)CCOC21CCN(Cc2cnn(CCS(=O)(=O)N(C)C)c2)CC1] | 1131 |
| 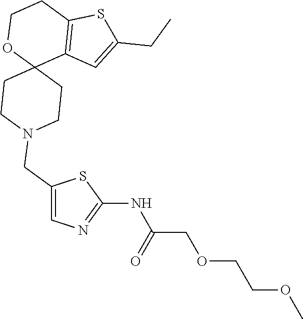 [SMILES: CCc1cc2c(s1)CCOC21CCN(Cc2cnc(NC(=O)COCCOC)s2)CC1] | 1132 |
| 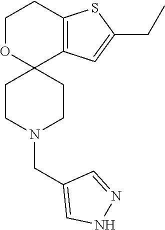 [SMILES: CCc1cc2c(s1)CCOC21CCN(Cc2cn[nH]c2)CC1] | 1133 |
| 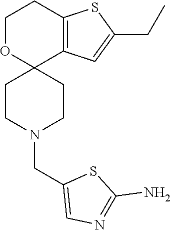 [SMILES: CCc1cc2c(s1)CCOC21CCN(Cc2cnc(N)s2)CC1] | 1134 |
| 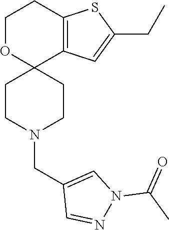 [SMILES: CCc1cc2c(s1)CCOC21CCN(Cc2cnn(C(C)=O)c2)CC1] | 1135 |
| 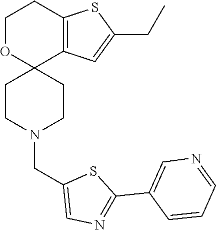 [SMILES: CCc1cc2c(s1)CCOC21CCN(Cc2cnc(-c3cccnc3)s2)CC1] | 1136 |
| 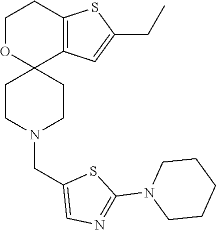 [SMILES: CCc1cc2c(s1)CCOC21CCN(Cc2cnc(N3CCCCC3)s2)CC1] | 1137 |
| 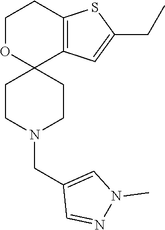 [SMILES: CCc1cc2c(s1)CCOC21CCN(Cc2cnn(C)c2)CC1] | 1138 |
| 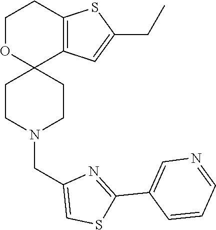 [SMILES: CCc1cc2c(s1)CCOC21CCN(Cc2csc(-c3cccnc3)n2)CC1] | 1139 |
| 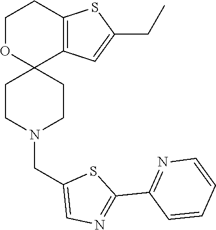 [SMILES: CCc1cc2c(s1)CCOC21CCN(Cc2cnc(-c3ccccn3)s2)CC1] | 1140 |
| 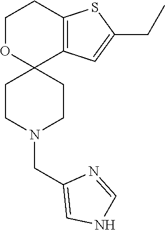 [SMILES: CCc1cc2c(s1)CCOC21CCN(Cc2c[nH]cn2)CC1] | 1141 |
| 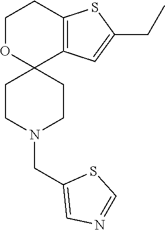 [SMILES: CCc1cc2c(s1)CCOC21CCN(Cc2cncs2)CC1] | 1142 |
| 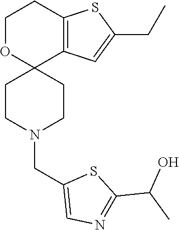 [SMILES: CCc1cc2c(s1)CCOC21CCN(Cc2cnc(C(C)O)s2)CC1] | 1143 |
| 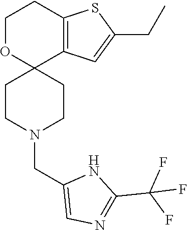 [SMILES: CCc1cc2c(s1)CCOC21CCN(Cc2cnc(C(F)(F)F)[nH]2)CC1] | 1144 |
| 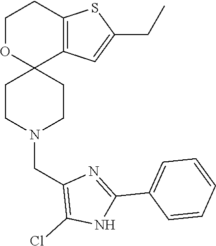 [SMILES: CCc1cc2c(s1)CCOC21CCN(Cc2nc(-c3ccccc3)[nH]c2Cl)CC1] | 1145 |
| 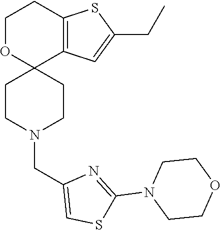 [SMILES: CCc1cc2c(s1)CCOC21CCN(Cc2csc(N3CCOCC3)n2)CC1] | 1146 |
| 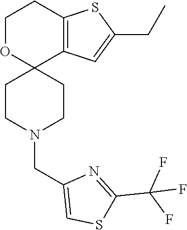 [SMILES: CCc1cc2c(s1)CCOC21CCN(Cc2csc(C(F)(F)F)n2)CC1] | 1147 |
| 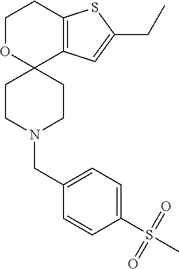 [SMILES: CCc1cc2c(s1)CCOC21CCN(Cc2ccc(S(C)(=O)=O)cc2)CC1] | 1148 |
| 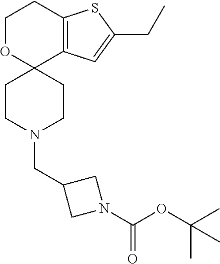 [SMILES: CCc1cc2c(s1)CCOC21CCN(CC2CN(C(=O)OC(C)(C)C)C2)CC1] | 1149 |
| 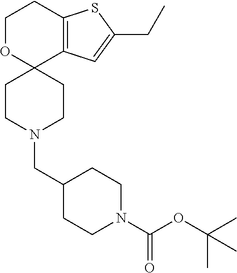 [SMILES: CCc1cc2c(s1)CCOC21CCN(CC2CCN(C(=O)OC(C)(C)C)CC2)CC1] | 1150 |
| 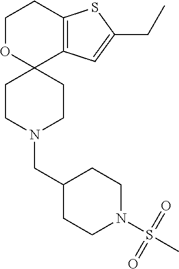 [SMILES: CCc1cc2c(s1)CCOC21CCN(CC2CCN(S(C)(=O)=O)CC2)CC1] | 1151 |
| 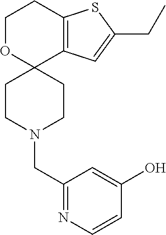 [SMILES: CCc1cc2c(s1)CCOC21CCN(Cc2cc(O)ccn2)CC1] | 1152 |
| 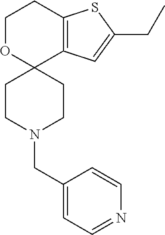 [SMILES: CCc1cc2c(s1)CCOC21CCN(Cc2ccncc2)CC1] | 1153 |
| 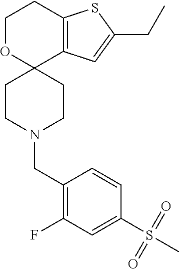 [SMILES: CCc1cc2c(s1)CCOC21CCN(Cc2ccc(S(C)(=O)=O)cc2F)CC1] | 1154 |
| 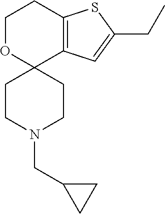 [SMILES: CCc1cc2c(s1)CCOC21CCN(CC2CC2)CC1] | 1155 |
| 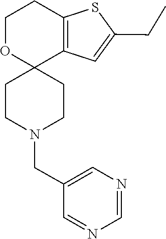 [SMILES: CCc1cc2c(s1)CCOC21CCN(Cc2cncnc2)CC1] | 1156 |
| 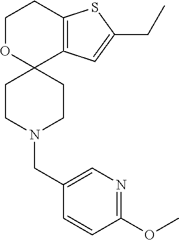 [SMILES: CCc1cc2c(s1)CCOC21CCN(Cc2ccc(OC)nc2)CC1] | 1157 |
| 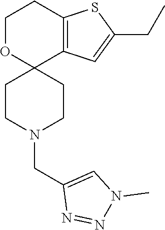 [SMILES: CCc1cc2c(s1)CCOC21CCN(Cc2cn(C)nn2)CC1] | 1158 |
| 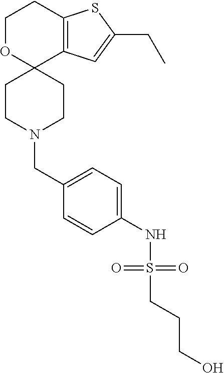 [SMILES: CCc1cc2c(s1)CCOC21CCN(Cc2ccc(NS(=O)(=O)CCCO)cc2)CC1] | 1159 |
| 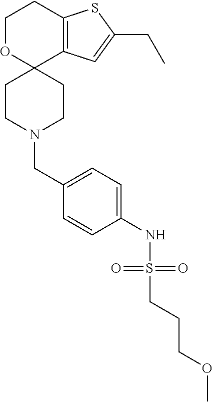 [SMILES: CCc1cc2c(s1)CCOC21CCN(Cc2ccc(NS(=O)(=O)CCCOC)cc2)CC1] | 1160 |
| 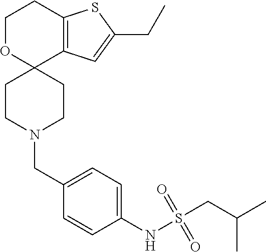 [SMILES: CCc1cc2c(s1)CCOC21CCN(Cc2ccc(NS(=O)(=O)CC(C)C)cc2)CC1] | 1161 |
| 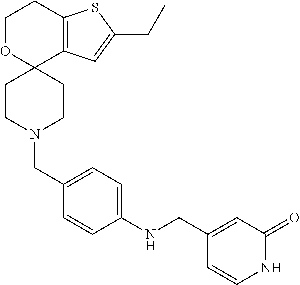 [SMILES: CCc1cc2c(s1)CCOC21CCN(Cc2ccc(NCc3cc[nH]c(=O)c3)cc2)CC1] | 1162 |
| 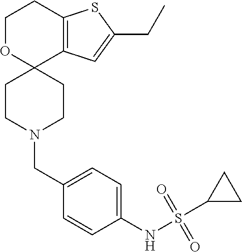 [SMILES: CCc1cc2c(s1)CCOC21CCN(Cc2ccc(NS(=O)(=O)C3CC3)cc2)CC1] | 1163 |
| 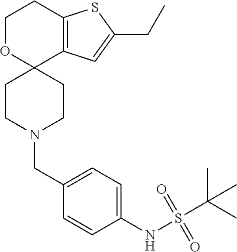 [SMILES: CCc1cc2c(s1)CCOC21CCN(Cc2ccc(NS(=O)(=O)C(C)(C)C)cc2)CC1] | 1164 |
| 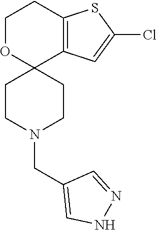 [SMILES: Clc1cc2c(s1)CCOC21CCN(Cc2cn[nH]c2)CC1] | 1165 |
| 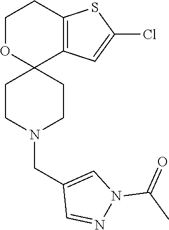 [SMILES: CC(=O)n1cc(CN2CCC3(CC2)OCCc2sc(Cl)cc23)cn1] | 1166 |
| 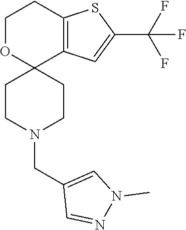 [SMILES: Cn1cc(CN2CCC3(CC2)OCCc2sc(C(F)(F)F)cc23)cn1] | 1167 |
| 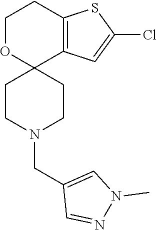 [SMILES: Cn1cc(CN2CCC3(CC2)OCCc2sc(Cl)cc23)cn1] | 1168 |
| 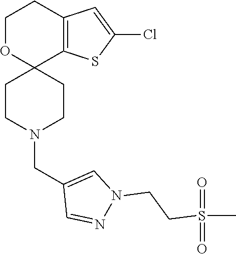 [SMILES: CS(=O)(=O)CCn1cc(CN2CCC3(CC2)OCCc2cc(Cl)sc23)cn1] | 1169 |
| 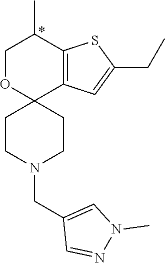 [SMILES: CCc1cc2c(s1)C(C)COC21CCN(Cc2cnn(C)c2)CC1] | 1170 |
| 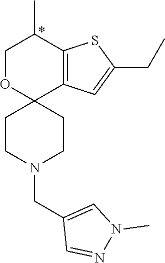 [SMILES: CCc1cc2c(s1)C(C)COC21CCN(Cc2cnn(C)c2)CC1] | 1171 |
| 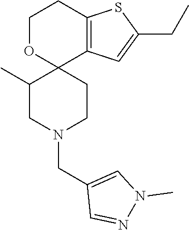 [SMILES: CCc1cc2c(s1)CCOC21CCN(Cc2cnn(C)c2)CC1C] | 1172 |
| 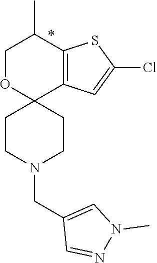 [SMILES: CC1COC2(CCN(Cc3cnn(C)c3)CC2)c2cc(Cl)sc21] | 1173 |
| 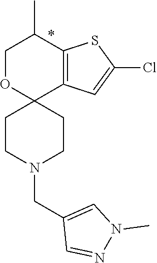 [SMILES: CC1COC2(CCN(Cc3cnn(C)c3)CC2)c2cc(Cl)sc21] | 1174 |
| 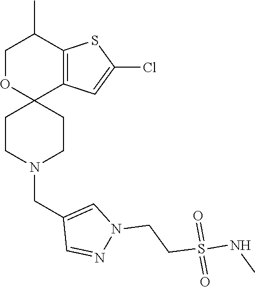 [SMILES: CNS(=O)(=O)CCn1cc(CN2CCC3(CC2)OCC(C)c2sc(Cl)cc23)cn1] | 1175 |
| 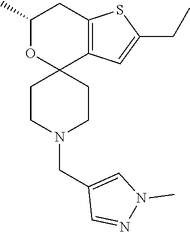 [SMILES: CCc1cc2c(s1)C[C@@H](C)OC21CCN(Cc2cnn(C)c2)CC1] | 1176 |
| 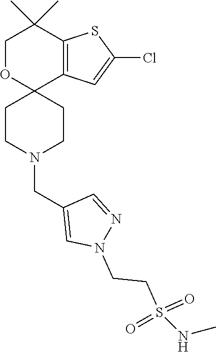 [SMILES: CNS(=O)(=O)CCn1cc(CN2CCC3(CC2)OCC(C)(C)c2sc(Cl)cc23)cn1] | 1177 |
| 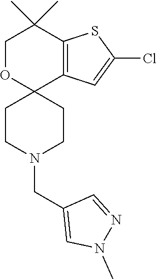 [SMILES: Cn1cc(CN2CCC3(CC2)OCC(C)(C)c2sc(Cl)cc23)cn1] | 1178 |
| 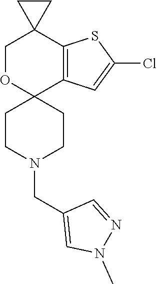 [SMILES: Cn1cc(CN2CCC3(CC2)OCC2(CC2)c2sc(Cl)cc23)cn1] | 1179 |
| 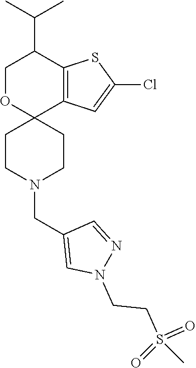 [SMILES: CC(C)C1COC2(CCN(Cc3cnn(CCS(C)(=O)=O)c3)CC2)c2cc(Cl)sc21] | 1180 |
| 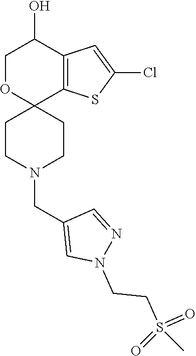 [SMILES: CS(=O)(=O)CCn1cc(CN2CCC3(CC2)OCC(O)c2cc(Cl)sc23)cn1] | 1181 |
| 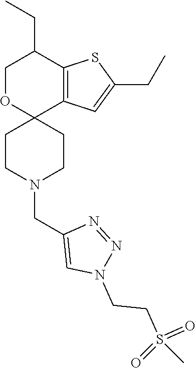 [SMILES: CCc1cc2c(s1)C(CC)COC21CCN(Cc2cn(CCS(C)(=O)=O)nn2)CC1] | 1182 |
| 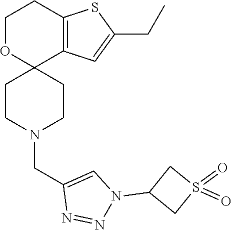 [SMILES: CCc1cc2c(s1)CCOC21CCN(Cc2cn(C3CS(=O)(=O)C3)nn2)CC1] | 1183 |

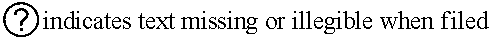

Some embodiments of the disclosure include derivatives of Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, or pharmaceutically acceptable salts of any of the foregoing. In some embodiments, the derivatives are silicon derivatives in which at least one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by silicon. In some embodiments, the derivatives are boron derivatives, in which at least one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by boron. In other embodiments, the derivatives are phosphorus derivatives, in which at least one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by phosphorus.

In some embodiments, the derivative is a silicon derivative in which one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by silicon or a silicon derivative (e.g., —Si(CH₃)₂— or —Si(OH)₂—). The carbon replaced by silicon may be a non-aromatic carbon. In other embodiments, a fluorine has been replaced by silicon derivative (e.g., —Si(CH₃)₃). In some embodiments, the silicon derivatives of the disclosure may include one or more hydrogen atoms replaced by deuterium. In some embodiments, a silicon derivative of compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, a tautomer thereof, a deuterated derivative of that compound or tautomer, or a pharmaceutically acceptable salt of any of the foregoing, may have silicon incorporated into a heterocycle ring.

In some embodiments, the derivative is a boron derivative in which one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by boron or a boron derivative.

In some embodiments, the derivative is a phosphorus derivative in which one carbon atom in a compound, tautomer, deuterated derivative, or pharmaceutically acceptable salt chosen from Compounds 1 to 1183 or compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing, has been replaced by phosphorus or a phosphorus derivative.

Another aspect of the disclosure provides pharmaceutical compositions comprising at least one compound, tautomer, deuterated derivative, or pharmaceutically acceptable salt according to any one formula chosen from Formulae I, Ia, Ib, Ic, Id, Ie, If, Ig, Ih, Ii, Ij, Ik, Il, and Im, and Compounds 1 to 1183, tautomers thereof, deuterated derivatives of those compounds or tautomers, and pharmaceutically acceptable salts of any of the foregoing. In some embodiments, the pharmaceutical composition comprising at least one compound, tautomer, deuterated derivative, or pharmaceutically acceptable salt chosen from Formulae Formulae I, Ia, Ib, Ic, Id, Ie, If, Ig, Ih, Ii, Ij, Ik, Il, and Im, and Compounds 1 to 1183, tautomers thereof, deuterated derivatives of those compounds or tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent. In some embodiments, a pharmaceutical composition comprising at least one compound, tautomer, deuterated derivative, or pharmaceutically acceptable salt chosen from Compounds 1 to 1183, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

### Compounds and Compositions for Use in Treatment

In some embodiments of the disclosure, the compounds and the pharmaceutical compositions described herein are used to treat FSGS and/or NDKD. In some embodiments, FSGS is mediated by APOL1. In some embodiments, NDKD is mediated by APOL1.

In some embodiments of the disclosure, the compounds and the pharmaceutical compositions described herein are used to treat cancer. In some embodiments, the cancer is mediated by APOL1.

In some embodiments of the disclosure, the compounds and the pharmaceutical compositions described herein are used to treat pancreatic cancer. In some embodiments, the pancreatic cancer is mediated by APOL1.

In some embodiments, the methods of the disclosure comprise administering to a patient in need thereof at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds and tautomers, and pharmaceutically acceptable salts of any of the foregoing. In some embodiments, the compound, tautomer, deuterated derivative, or pharmaceutically acceptable salt is chosen from Compounds 1 to 1183, tautomer thereof, deuterated derivatives of those compounds or tautomers, and pharmaceutically acceptable salts of any of the foregoing. In some embodiments, said patient in need thereof possesses APOL1 genetic variants, i.e., G1: S342G:I384M and G2: N388del:Y389del.

Another aspect of the disclosure provides methods of inhibiting APOL1 activity comprising contacting said APOL1 with at least one compound, tautomer, deuterated derivative, or pharmaceutically acceptable salt chosen from compounds of Formulae I, Ia, Ib, Ic, Id, Ie, If, Ig, Ih, Ii, Ij, Ik, Il, and Im, tautomers thereof, deuterated derivatives of those compounds or tautomers, and pharmaceutically acceptable salts of any of the foregoing. In some embodiments, the methods of inhibiting APOL1 activity comprise contacting said APOL1 with at least one compound, tautomer, deuterated derivative, or pharmaceutically acceptable salt chosen from Compounds 1 to 1183, tautomers thereof, deuterated derivatives of those compounds or tautomers, and pharmaceutically acceptable salts of any of the foregoing.

### EXAMPLES

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae I, Ia, Ib, Ic, Id, Ie, If, Ig, Ih, Ii, Ij, Ik, Il, and Im, Compounds 1 to 1183, tautomers thereof, deuterated derivatives of those compounds and tautomers, and pharmaceutically acceptable salts of any of the foregoing, the following abbreviations are used:

### Abbreviations

AcOH=acetic acid
ARP=assay ready plate
Boc₂O=di-tert-butyl decarbonate
n-BuLi=n-butyllithium
CFL=compact fluorescent lamp
18-crown-6=1,4,7,10,13,16-hexaoxacyclooctadecane
DAST=diethylaminosulfur trifluoride
DBDMH=1,3-dibromo-5,5-dimethylhydantoin
DBU=1,8-diazabicyclo[5.4.0]undec-7-ene
DCE=1,2-dichloroethane
DCM=dichloromethane
Deoxo-fluor=bis(2-methoxyethyl)aminosulfur trifluoride
DIBAL-H=diisobutylaluminum hydride
DIPEA or DIEA=N,N-diisopropylethylamine or N-ethyl-N-isopropyl-propan-2-amine
DMAP=dimethylamino pyridine
DMA=dimethyl acetamide
DME=dimethoxyethane
DMEM=Dulbecco's modified Eagle's medium
DMF=dimethylformamide
DMPU=N,N′-dimethylpropyleneurea
DMSO=dimethyl sulfoxide
dppf=1,1′-bis(diphenylphosphino)ferrocene
ESI=electrospray ionization
EtOAc=ethyl acetate
EtOH=ethanol
Et₂O=diethyl ether
Et₂NH=diethylamine
Et₃N=triethylamine
FA=formamide
FBS=fetal bovine serum
GF-AFC=glycyl-phenylalanylamino fluorocoumarin
HATU=[dimethylamino(triazolo[4,5-b]pyridin-3-yloxy)methylene]-dimethyl-ammonium (phosphorus hexafluoride ion)
HDMC=N-[(5-chloro-3-oxido-1H-benzotriazol-1-yl)-4-morpholinylmethylene]-N-methylmethanaminium hexafluorophosphate
HPLC=high-performance liquid chromatography
IPA=isopropyl alcohol
(Ir[dF(CF₃)ppy]₂(dtbpy))PF₆=[4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
[Ir{dFCF3ppy}2(bpy)]PF6=(2,2′-Bipyridine)bis[3,5-difluoro-2-[5-(trifluoromethyl)-2-pyridinyl-kN][phenyl-kC]iridium(III) hexafluorophosphate
LDA=lithium diisopropyl amide
LED=light emitting diode
MeCN or ACN=acetonitrile
MeI=methyl iodide
MeMgCl=methylmagnesium chloride
MeOH=methanol
MsOH=methanesulfonic acid
MTBE or TBME=methyl tert-butyl ether
n-BuLi=n-butyllithium
n-BuOH=1-butanol
NaOtBu=Sodium tert-butoxide
NBS=N-bromosuccinimide
NCS=N-chlorosuccinimide
NHPI=N-hydroxyphthalimide
NIS=N-iodosuccinimide
NMM=N-methyl morpholine
NMP=N-methyl pyrrolidine
PBS=phosphate-buffered saline
Pd(dppf)₂Cl₂=[1,1′-bis(diphenylphosphino)ferrocene]dichloropalladium(II)
PdCl₂(PPh₃)₂=bis(triphenylphosphine)palladium(II) dichloride
pet=petroleum
PP=polypropylene
PPh₃=triphenylphosphine
PhMgCl=Phenylmagnesium chloride
PSCBH=polymer-supported cyanoborohydride
PTSA=p-toluenesulfonic acid monohydrate
RBF=round-bottom flask
SFC=supercritical fluid chromatography
SPE=solid phase extraction
STAB=sodium triacetoxyborohydride
T3P=2,4,6-tripropyl-1,3,5,2,4,6-trioxatriphosphorinane-2,4,6-trioxide
TBAF=tetra-n-butylammonium fluoride
TBS=tert-butyldimethylsilyl
TBSCl=tert-butyldimethylsilyl chloride
TBTA=tris((1-benzyl-4-triazolyl)methyl)amine
TBuONO=tert-butyl nitrite
tBuXPhos Pd G1=[2-(Di-tert-butylphosphino)-2′,4′,6′-triisopropyl-1,1′-biphenyl][2-(2-aminoethyl)phenyl)]palladium(II) chloride
tBuOH=tert-butanol
TEA=triethylamine
tet=tetracycline
TFA or TFAA=trifluoroacetic acid
TfOH=trifluoromethanesulfonic acid
THF=tetrahydrofuran
T3P=2,4,6-tripropyl-1,3,5,2,4,6-trioxatriphosphorinane-2,4,6-trioxide
2-Me-THF=2-methyltetrahydrofuran
THP=tetrahydropyran
TLC=thin layer chromatography
TMS-OTf=trifluoromethanesulfonic acid trimethylsilylester
TMSCl=trimethylsilyl chloride
TMSCF₃=trifluoromethyltrimethylsilane
XPhos Pd G3=(2-Dicyclohexylphosphino-2′,4′,6′-triisopropyl-1,1′-biphenyl)[2-(2′-amino-1,1′-biphenyl)]palladium(II) methanesulfonate

### Example 1. Synthesis of Compounds

All the specific and generic compounds, and the intermediates disclosed for making those compounds, are considered to be part of the disclosure disclosed herein.

### Preparation S1

### (2′S,4R)-2-ethyl-2′-methyl-1′-prop-2-ynyl-spiro[6,7-dihydrothieno[3,2-c]pyran-4,4′-piperidine](S1)

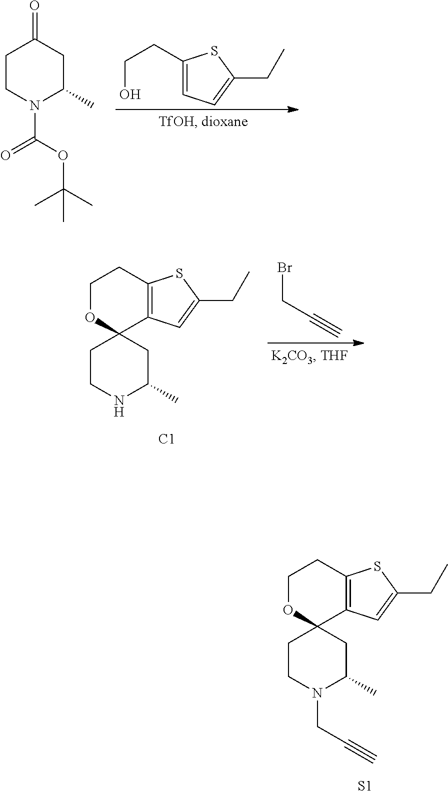

### Step 1. Synthesis of (2′S,4R)-2-ethyl-2′-methyl-spiro[6,7-dihydrothieno[3,2-c]pyran-4,4′-piperidine (C1)

To a solution of tert-butyl (2S)-2-methyl-4-oxo-piperidine-1-carboxylate (10 g, 46.89 mmol) and 2-(5-ethyl-2-thienyl)ethanol (7 g, 42.56 mmol) in dioxane (80 mL) at 0° C. was added a solution of trifluoromethanesulfonic acid (12.4 mL, 140.1 mmol) in dioxane (20 mL) over 15 min. The reaction was stirred at the same temperature for 1 h, and then warmed to ambient temperature and stirred for 3.5 h. The mixture was diluted with water (300 mL) and ethyl acetate (200 mL). The organic layer was separated, and the aqueous layer was extracted with EtOAc (100 mL×2). The combined organic extracts were dried over Na₂SO₄, filtered and concentrated in vacuo. The crude was dissolved in DCM and filtered through a plug of silica gel (250 g) eluting with DCM followed by 25% MeOH/DCM to afford the desired product. The product was dissolved in Et₂O (500 mL) and heated at reflux for 15 min. After cooling down to ambient temperature, heptane (500 mL) was added, and the solids were filtered, washed with heptane and dried to furnish the title compound as a grey solid (triflate salt) (11.36 g, 67%). ¹H NMR (400 MHz, DMSO-d6) δ 6.46 (d, J=1.1 Hz, 1H), 3.96-3.79 (m, 2H), 3.42-3.34 (m, 1H), 3.27-3.00 (m, 2H), 2.80-2.63 (m, 4H), 2.01-1.87 (m, 3H), 1.79 (dd, J=14.5, 12.2 Hz, 1H), 1.24-1.16 (m, 6H). LCMS m/z 252.01[M+H]⁺.

### Step 2. Synthesis of (2′S,4R)-2-ethyl-2′-methyl-1′-prop-2-ynyl-spiro[6,7-dihydrothieno[3,2-c]pyran-4,4′-piperidine](S1)

To a mixture of (2′S,4R)-2-ethyl-2′-methyl-spiro[6,7-dihydrothieno[3,2-c]pyran-4,4′-piperidine] C1 (triflate salt) (76.7 g, 187.7 mmol) and K₂CO₃ (46 g, 332.8 mmol) in THF (900 mL) was added propargyl bromide (21 mL, 188.5 mmol, 80% w/w solution in toluene). The resulting mixture was heated at 45° C. for 5 h, and then cooled down to room temperature and stirred overnight. The reaction was diluted with EtOAc (350 mL), brine (150 mL) and 2M aqueous Na₂S₂O₃ solution (25 mL). The organic layer was separated and the aqueous layer was extracted with EtOAc (100 mL). The combined organic extracts were dried over Na₂SO₄, filtered and concentrated in vacuo. The crude was triturated with Et₂O. The resulting colorless solids were filtered and dried to afford the S1 (55.7 g, 92%). ¹H NMR (300 MHz, Chloroform-d) δ 6.47 (d, J=1.1 Hz, 1H), 4.05-3.85 (m, 2H), 3.67 (dd, J=17.3, 2.4 Hz, 1H), 3.44 (dd, J=17.3, 2.4 Hz, 1H), 3.00-2.64 (m, 7H), 2.25 (t, J=2.4 Hz, 1H), 2.03-1.81 (m, 3H), 1.68 (dd, J=13.8, 11.5 Hz, 1H), 1.28 (t, J=7.5 Hz, 3H), 1.10 (d, J=6.3 Hz, 3H). LCMS m/z 290.11 [M+H]⁺.

### Preparation S2

### (2′S,4R)-1′-[[1-(azetidin-3-yl)triazol-4-yl]methyl]-2-ethyl-2′-methyl-spiro[6,7-dihydrothieno[3,2-c]pyran-4,4′-piperidine](S2)

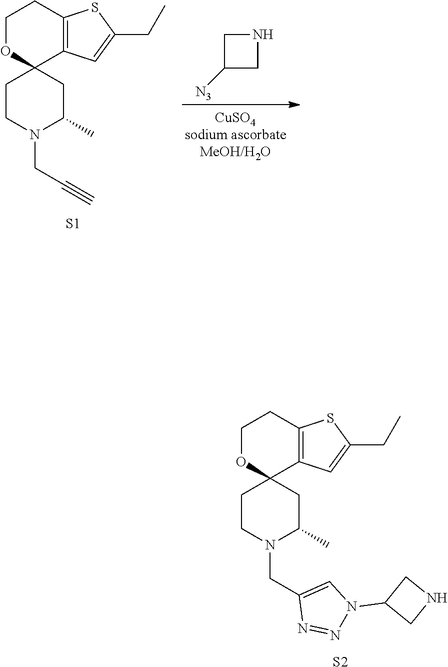

Compound S2 was prepared from S1 and 3-azidoazetidine, employing the same method as described for Compound 1.

### Compound 1

### 2-[3-[4-[[(2′S,4R)-2-ethyl-2′-methyl-spiro[6,7-dihydrothieno[3,2-c]pyran-4,4′-piperidine]-1′-yl]methyl]triazol-1-yl]azetidin-1-yl]sulfonylethanol (1)

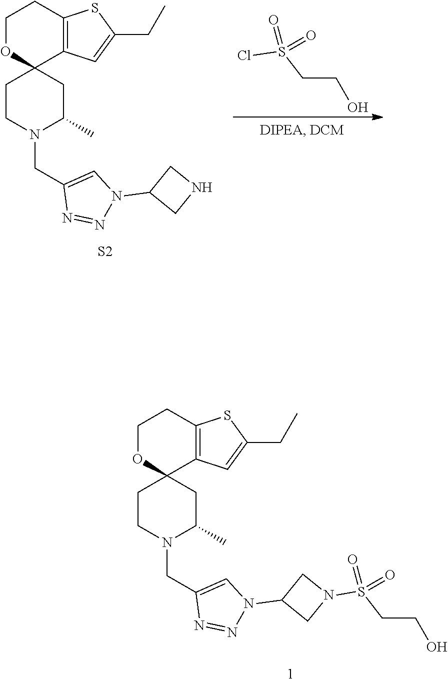

### Preparation of 2-[3-[4-[[(2′S,4R)-2-ethyl-2′-methyl-spiro[6,7-dihydrothieno[3,2-c]pyran-4,4′-piperidine]-1′-yl]methyl]triazol-1-yl]azetidin-1-yl]sulfonylethanol (1)

To a solution of (2′S,4R)-1′-[[1-(azetidin-3-yl)triazol-4-yl]methyl]-2-ethyl-2′-methyl-spiro[6,7-dihydrothieno[3,2-c]pyran-4,4′-piperidine]S2 (10 mg, 0.026 mmol) in DCM (0.65 mL) was added 2-hydroxyethane-1-sulfonyl chloride (4.1 mg, 0.028 mmol) and DIPEA (9.0 μL, 0.052 mmol). The reaction was stirred at room temperature for 2 hours. The volatile was then removed and the crude was purified by reversed phase chromatography (C18 column; Gradient: MeCN in H₂O with 0.1% TFA) to afford 1 (3.1 mg, 24%). ¹H NMR (300 MHz, Methanol-d4) δ 8.44 (s, 1H), 6.51 (s, 1H), 5.57 (ddd, J=13.5, 7.8, 5.7 Hz, 1H), 4.80-4.37 (m, 6H), 3.97 (t, J=5.9 Hz, 2H), 3.88 (p, J=6.0, 5.5 Hz, 2H), 3.57 (d, J=7.9 Hz, 1H), 3.40 (q, J=6.4, 5.9 Hz, 4H), 2.88-2.56 (m, 4H), 2.28-1.85 (m, 4H), 1.58 (d, J=6.4 Hz, 3H), 1.25 (t, J=7.5 Hz, 3H). LCMS m/z 496.14 [M+H]⁺.

### Compounds 2-5

Compounds 2-5 (see Table 2) were prepared from S2 using the appropriate sulfonyl chlorides employing the same method as described for Compound 1. All sulfonyl chlorides were obtained from commercial sources, unless noted otherwise. Any modifications to methods are noted in Table 2 and accompanying footnotes.

TABLE 2
Structure and physicochemical data for compounds 2-5

| | | | | ¹H NMR; LCMS |
|---|---|---|---|---|
| Compd | Structure | Amine | Method | m/z [M + H]⁺ |
| 2 | 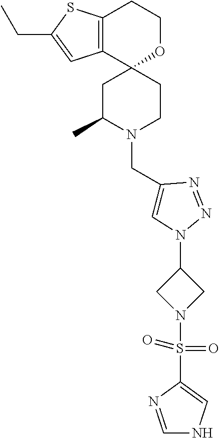 [SMILES: CCc1cc2c(s1)CCO[C@@]21CCN(Cc2cn(C3CN(S(=O)(=O)c4c[nH]cn4)C3)nn2)[C@@H](C)C1] | 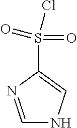 | Compound 1 | LCMS m/z [M + H]⁺ 518.06 |
| 3 | 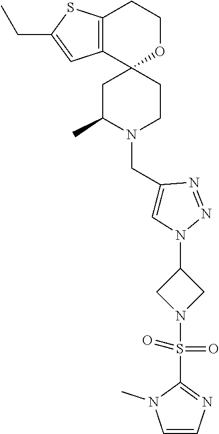 [SMILES: CCc1cc2c(s1)CCO[C@@]21CCN(Cc2cn(C3CN(S(=O)(=O)c4nccn4C)C3)nn2)[C@@H](C)C1] | 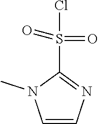 | Compound 1 | LCMS m/z [M + H]⁺ 532.23 |
| 4 | 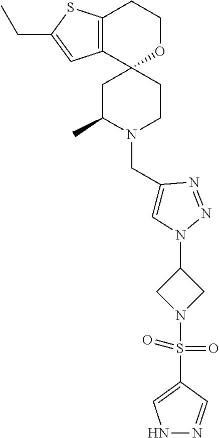 [SMILES: CCc1cc2c(s1)CCO[C@@]21CCN(Cc2cn(C3CN(S(=O)(=O)c4cn[nH]c4)C3)nn2)[C@@H](C)C1] | 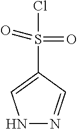 | Compound 1 | LCMS m/z [M + H]⁺ 518.22 |
| 5 | 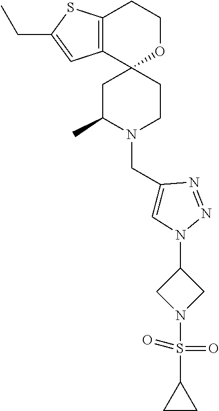 [SMILES: CCc1cc2c(s1)CCO[C@@]21CCN(Cc2cn(C3CN(S(=O)(=O)C4CC4)C3)nn2)[C@@H](C)C1] | 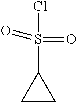 | Compound 1 | LCMS m/z [M + H]⁺ 492.2 |

### Preparation S3

### 2-azido-N-(2-hydroxyethyl)ethane-1-sulfonamide (S3)

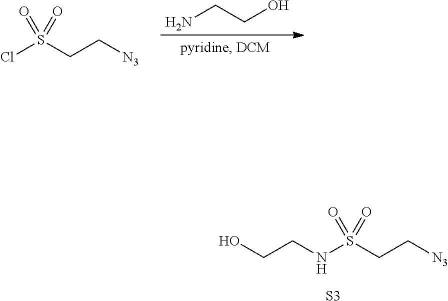

To a solution of 2-azidoethanesulfonyl chloride (60 mg, 0.35 mmol) in DCM (2 mL) was added pyridine (60 μL, 0.7418 mmol) and 2-aminoethanol (30 μL, 0.50 mmol). The mixture was stirred at room temperature (“rt”) for 2 hours (“h”). The volatile was removed to afford the title compound. The crude was used directly without further purification.

### Preparation S4

### 2-azido-N-(2-hydroxypropyl)ethane-1-sulfonamide (S4)

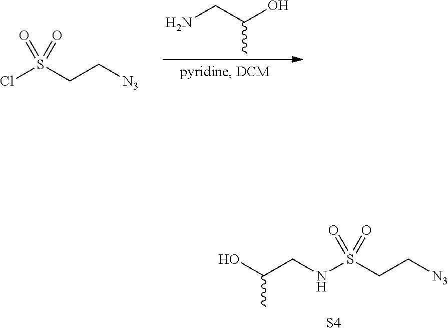

Compound S4 was prepared from 2-azidoethanesulfonyl chloride and 1-aminopropan-2-ol using the same method as described for S3. The crude title compound was used directly without further purification.

### Compound 6

### 2-[4-[[(2′S,4R)-2-ethyl-2′-methyl-spiro[6,7-dihydrothieno[3,2-c]pyran-4,4′-piperidine]-1′-yl]methyl]triazol-1-yl]-N-(2-hydroxyethyl)ethanesulfonamide (6)

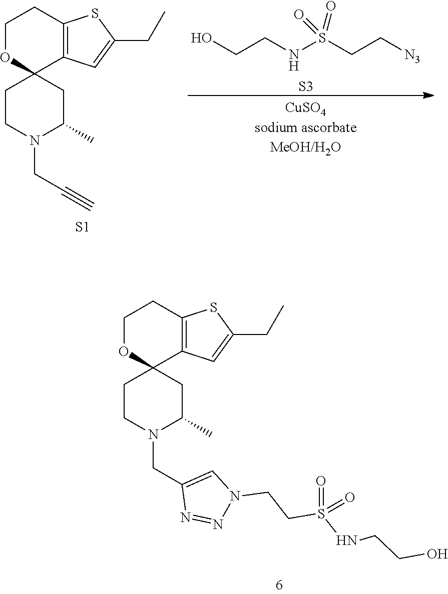

### Preparation of 2-[4-[[(2′S,4R)-2-ethyl-2′-methyl-spiro[6,7-dihydrothieno[3,2-c]pyran-4,4′-piperidine]-1′-yl]methyl]triazol-1-yl]-N-(2-hydroxyethyl)ethanesulfonamide (6)

To a solution of 2-azido-N-(2-hydroxyethyl)ethane-1-sulfonamide (67.9 mg, 0.35 mmol) in MeOH (2 mL) and H₂O (0.2 mL) was added (2′S)-2-ethyl-2′-methyl-1′-prop-2-ynyl-spiro[6,7-dihydrothieno[3,2-c]pyran-4,4′-piperidine]S1 (50 mg, 0.17 mmol), sodium ascorbate (37 mg, 0.21 mmol), and CuSO₄ (1 mg, 0.006 mmol). The mixture was heated at 50° C. for 1 hour. After cooling down to room temperature, the mixture was diluted with DCM and H₂O. The pH of the aqueous layer was adjusted to pH 10. The organic layer was separated and the aqueous layer was extracted with DCM. The combined organic extracts were concentrated in vacuo. The crude was purified by reversed phase chromatography (C18 column; Gradient: MeCN in H₂O with 0.1% HCl) to afford the title compound (13.9 mg, 17%). ¹H NMR (400 MHz, DMSO-d6) δ 8.54 (s, 1H), 7.53 (s, 2H), 7.05 (s, 1H), 5.29 (t, J=7.3 Hz, 2H), 4.45-4.08 (m, 5H), 3.73 (s, 6H), 3.26-3.04 (m, 5H), 2.36-1.96 (m, 4H), 1.73-1.61 (m, 6H). LCMS m/z 484.12[M+H]⁺.

### Compound 7

### 2-[4-[[(2′S,4R)-2-ethyl-2′-methyl-spiro[6,7-dihydrothieno[3,2-c]pyran-4,4′-piperidine]-1′-yl]methyl]triazol-1-yl]-N-(2-hydroxypropyl)ethanesulfonamide (7)

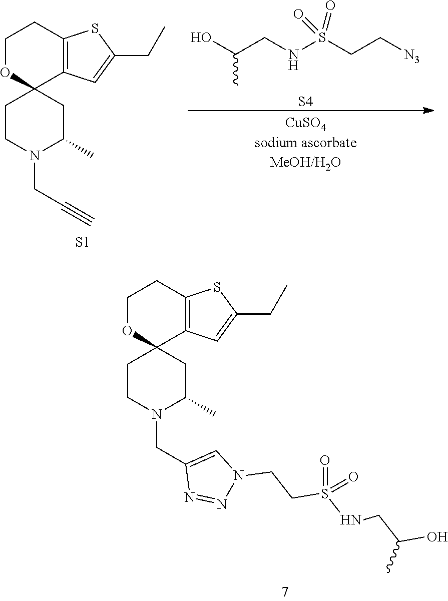

### Preparation of 2-[4-[[(2′S,4R)-2-ethyl-2′-methyl-spiro[6,7-dihydrothieno[3,2-c]pyran-4,4′-piperidine]-1′-yl]methyl]triazol-1-yl]-N-(2-hydroxypropyl)ethanesulfonamide (7)

Compound 7 was prepared from (2′S)-2-ethyl-2′-methyl-1′-prop-2-ynyl-spiro[6,7-dihydrothieno[3,2-c]pyran-4,4′-piperidine] and 2-azido-N-(2-hydroxypropyl)ethane-1-sulfonamide using the same method as described for Compound 6. The crude was purified by reversed phase chromatography (C18 column; Gradient: MeCN in H₂O with 0.1% HCl) to afford the title compound (7.1 mg, 8%). ¹H NMR (400 MHz, DMSO-d6) δ 8.53 (d, J=3.5 Hz, 1H), 7.71 (t, J=6.1 Hz, 1H), 7.05 (s, 1H), 5.26 (q, J=6.9 Hz, 2H), 4.40-4.06 (m, 6H), 3.45-3.25 (m, 2H), 3.46-2.92 (m, 7H), 2.35-1.93 (m, 5H), 1.75-1.53 (m, 9H). LCMS m/z 498.19 [M+H]⁺.

### Compound 8

### (2′S,4R)-2-ethyl-2′-methyl-1′-(1H-triazol-4-ylmethyl)spiro[6,7-dihydrothieno[3,2-c]pyran-4,4′-piperidine](8)

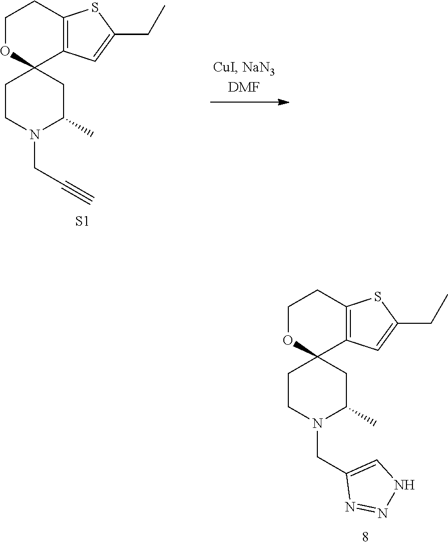

### Preparation of (2′S,4R)-2-ethyl-2′-methyl-1′-(1H-triazol-4-ylmethyl)spiro[6,7-dihydrothieno[3,2-c]pyran-4,4′-piperidine](8)

To a solution of (2′S,4R)-2-ethyl-2′-methyl-1′-prop-2-ynyl-spiro[6,7-dihydrothieno[3,2-c]pyran-4,4′-piperidine](40 mg, 0.14 mmol) in DMF (2 mL) was added sodium azide (30 mg, 0.46 mmol) and CuI (30 mg, 0.16 mmol). The mixture was stirred at rt overnight. The mixture was filtered and the filtrated was concentrated. The crude was purified by reversed phase chromatography (C18 column; Gradient: MeCN in H₂O with 0.1% HCl) to afford the title compound (4.6 mg, 9%). ¹H NMR (400 MHz, DMSO-d6) δ 8.53 (d, J=3.5 Hz, 1H), 7.71 (t, J=6.1 Hz, 1H), 7.05 (s, 1H), 5.26 (q, J=6.9 Hz, 2H), 4.40-4.06 (m, 6H), 3.45-3.25 (m, 2H), 3.46-2.92 (m, 7H), 2.35-1.93 (m, 5H), 1.75-1.53 (m, 9H). LCMS m/z 333.16 [M+H]⁺.

### Preparation S5 and S6

### (2′R,4R)-2-ethyl-2′-methyl-spiro[6,7-dihydrothieno[3,2-c]pyran-4,4′-piperidine](S5)

### (2′R,4S)-2-ethyl-2′-methyl-spiro[6,7-dihydrothieno[3,2-c]pyran-4,4′-piperidine](S6)

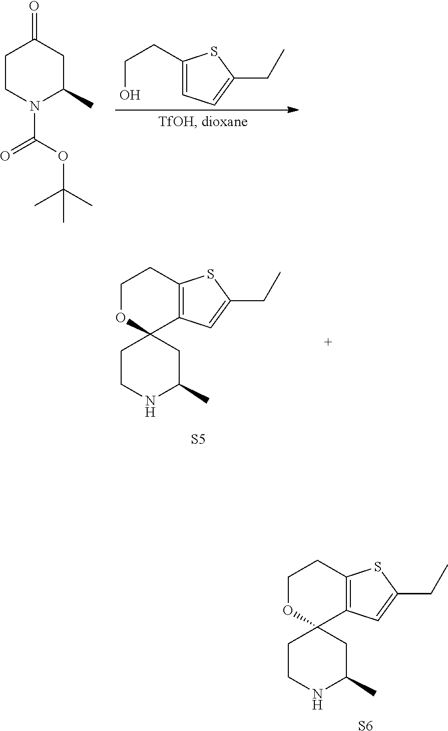

### Preparation of (2′R,4R)-2-ethyl-2′-methyl-spiro[6,7-dihydrothieno[3,2-c]pyran-4,4′-piperidine](S5) and (2′R,4S)-2-ethyl-2′-methyl-spiro[6,7-dihydrothieno[3,2-c]pyran-4,4′-piperidine](S6)

To a solution of 2-(5-ethyl-2-thienyl)ethanol (1.0 g, 6.4 mmol) and tert-butyl (2R)-2-methyl-4-oxo-piperidine-1-carboxylate (1.5 g, 7.03 mmol) in dioxane (15 mL) was added slowly a solution of trifluoromethanesulfonic acid (1.8 mL, 20.34 mmol) in dioxane (5 mL). The mixture was stirred at rt overnight. The mixture was diluted with H₂O and EtOAc. The organic layer was separated and the aqueous layer was extracted with EtOAc (×2). The combined organic extracts were dried over Na₂SO₄, filtered and concentrated in vacuo. The crude was purified to afford the title compound S5 (2.0 g, 23% purity, 18%) and S6 (2.0 g, 77% purity, 60%) as triflate salts. LCMS m/z 252.11 [M+H]⁺.

### Compound 9

### (2′R,4R)-2-ethyl-2′-methyl-1′-[[1-(2-methylsulfonylethyl)triazol-4-yl]methyl]spiro[6,7-dihydrothieno[3,2-c]pyran-4,4′-piperidine](9)

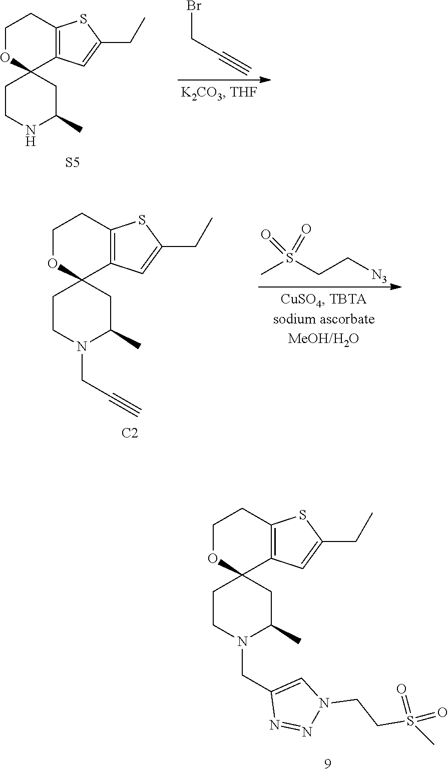

### Step 1. Synthesis of (2R,4R)-2′-ethyl-2-methyl-1-(prop-2-yn-1-yl)-6′,7′-dihydrospiro[piperidine-4,4′-thieno[3,2-c]pyran] (C2)

To a solution of (2′R,4R)-2-ethyl-2′-methyl-spiro[6,7-dihydrothieno[3,2-c]pyran-4,4′-piperidine]S5 (triflate salt) (3.0 g, 7.473 mmol) in THF (35 mL) was added K₂CO₃ (2.0 g, 14.47 mmol) and propargyl bromide (930 μL, 80% w/w solution in toluene, 8.35 mmol). The mixture was heated at 45° C. for 8 hour, and then cooled down to room temperature and stirred overnight. The reaction was diluted with EtOAc (350 mL), brine (150 mL) and 2 M aqueous solution of sodium thiosulfate (25 mL). The organic layer was separated and the aqueous layer was extracted with EtOAc (100 mL). The combined organic extracts were dried over Na₂SO₄, filtered and concentrated in vacuo. The residue was triturated with Et₂O. The resulting colorless solids were filtered off, and the filtrate was concentrated in vacuo to afford the title compound as a light brown oil (2.1 g, 74%). The product was used directly without further purification. LCMS m/z 290.11 [M+H]⁺.

### Step 2. Synthesis of (2′R,4R)-2-ethyl-2′-methyl-1′-[[1-(2-methylsulfonylethyl)triazol-4-yl]methyl]spiro[6,7-dihydrothieno[3,2-c]pyran-4,4′-piperidine](9)

To a solution of (2′R)-2-ethyl-2′-methyl-1′-prop-2-ynyl-spiro[6,7-dihydrothieno[3,2-c]pyran-4,4′-piperidine] C2 (1.0 g, 3.46 mmol, 23% purity) in MeOH (12.75 mL) and H₂O (4.25 mL) was added CuSO₄ (30 mg, 0.19 mmol), TBTA (110 mg, 0.21 mmol), and sodium ascorbate (619 mg, 3.52 mmol). The mixture was stirred at room temperature, and then a solution of 1-azido-2-methylsulfonyl-ethane (600 mg, 4.02 mmol) in MeOH (2 mL) was added drop-wise. After addition, the mixture was stirred for 90 min. The mixture was concentrated in vacuo. The residue was suspended in EtOAc and filtered. The filtrate was washed with a mixture of sat. NaHCO₃ solution and H₂O (1:1, 50 mL). The aqueous wash was back-extracted with EtOAc (25 mL). The combined organic extracts were dried over Na₂SO₄, filtered and concentrated in vacuo. The crude was purified by MPLC on silica gel (0-10% MeOH in DCM) to afford the title compound as a colorless glass (40 mg, 48%). ¹H NMR (300 MHz, Chloroform-d) δ 8.18 (s, 1H), 6.51 (s, 1H), 4.92 (t, J=6.6 Hz, 2H), 4.13 (s, 2H), 3.91 (td, J=6.6, 5.9, 3.3 Hz, 2H), 3.77 (t, J=6.6 Hz, 2H), 3.57-3.37 (m, 1H), 3.35-3.17 (m, 1H), 2.99 (d, J=12.0 Hz, 1H), 2.87 (s, 3H), 2.82-2.64 (m, 4H), 2.26 (td, J=15.7, 15.3, 4.8 Hz, 2H), 2.07-1.80 (m, 2H), 1.49 (d, J=6.8 Hz, 3H), 1.26 (t, J=7.5 Hz, 3H). LCMS m/z 440.27 [M+H]⁺.

### Compound 10

### (2′R,4S)-2-ethyl-2′-methyl-1′-[[1-(2-methylsulfonylethyl)triazol-4-yl]methyl]spiro[6,7-dihydrothieno[3,2-c]pyran-4,4′-piperidine](10)

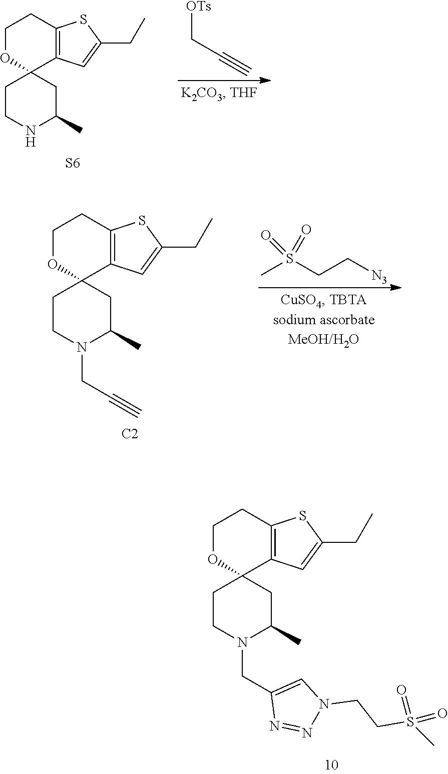

### Step 1. Synthesis of (2′R,4S)-2-ethyl-2′-methyl-1′-prop-2-ynyl-spiro[6,7-dihydrothieno[3,2-c]pyran-4,4′-piperidine] (C2)

To a solution of (2′R,4S)-2-ethyl-2′-methyl-spiro[6,7-dihydrothieno[3,2-c]pyran-4,4′-piperidine](triflate salt)) S6 (1.14 g, 2.460 mmol) in DMF (2 mL) and THF (8 mL) was added potassium carbonate (1 g, 7.24 mmol) and prop-2-ynyl 4-methylbenzenesulfonate (500 μL, 2.90 mmol). The mixture was stirred at 30° C. overnight. The reaction was diluted with EtOAc (30 mL), H₂O (20 mL) and brine (10 mL). The organic layer was separated, and the aqueous layer was extracted with EtOAc (50 mL). The combined organic extracts were washed with brine, dried over Na₂SO₄, filtered and concentrated in vacuo. The crude was purified using silica gel chromatography (10-35% EtOAc in hexanes) to afford C2 (350 mg, 44%). ¹H NMR (300 MHz, Chloroform-d) δ 6.41 (d, J=1.1 Hz, 1H), 3.96-3.74 (m, 2H), 3.47 (ddd, J=71.3, 17.3, 2.4 Hz, 2H), 2.98-2.48 (m, 7H), 1.92-1.72 (m, 3H), 1.60 (dd, J=13.8, 11.5 Hz, 1H), 1.21 (t, J=7.5 Hz, 4H), 1.03 (d, J=6.3 Hz, 3H). LCMS m/z 290.11 [M+H]⁺.

### Step 2. Synthesis of (2′R,4S)-2-ethyl-2′-methyl-1′-[[1-(2-methylsulfonylethyl)triazol-4-yl]methyl]spiro[6,7-dihydrothieno[3,2-c]pyran-4,4′-piperidine](10)

To a solution of (2′R,4S)-2-ethyl-2′-methyl-1′-prop-2-ynyl-spiro[3a,6,7,7a-tetrahydrothieno[3,2-c]pyran-4,4′-piperidine] C2 (350 mg, 1.20 mmol) in MeOH (8 mL) and H₂O (2 mL) was added CuSO₄ (5 mg, 0.031 mmol), TBTA (25 mg, 0.047 mmol), and sodium ascorbate (300 mg, 1.70 mmol). The mixture was stirred at rt, and then a solution of 1-azido-2-methylsulfonyl-ethane (200 mg, 1.34 mmol) in MeOH (2 mL) was added dropwise via an addition funnel. The mixture was stirred at room temperature for 2 h. The reaction was filtered and the filtrate was concentrated in vacuo. The residue was dissolved in EtOAc (150 mL) and H₂O (100 mL). The organic layer was separated and the aqueous layer was extracted with EtOAc (150 mL×2). The combined organic extracts were dried over Na₂SO₄, filtered, and concentrated in vacuo to give a tan solid. Purification by silica gel chromatography (0-40% EtOAc in hexanes) afforded 10 (448 mg, 81%). ¹H NMR (300 MHz, Chloroform-d) δ 7.66 (s, 1H), 6.40 (d, J=1.2 Hz, 1H), 4.81 (t, J=6.4 Hz, 2H), 3.98 (d, J=14.6 Hz, 1H), 3.89-3.65 (m, 5H), 2.72-2.46 (m, 10H), 1.85-1.57 (m, 4H), 1.26-1.11 (m, 6H). LCMS m/z 439.17[M+H]⁺.

### Compound 11

### (2′S,4R)-2-ethyl-2′-methyl-1′-[[1-(2-methylsulfonylethyl)triazol-4-yl]methyl]spiro[6,7-dihydrothieno[3,2-c]pyran-4,4′-piperidine](11)

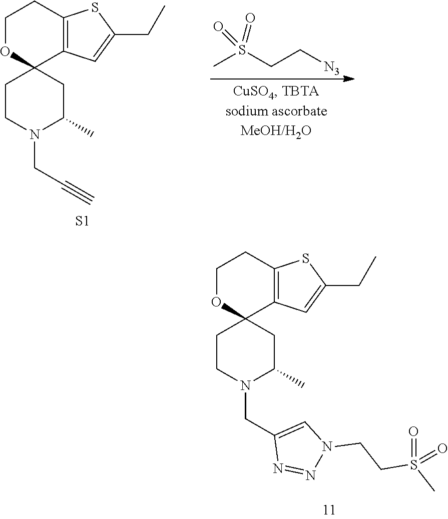

### Preparation of (2′S,4R)-2-ethyl-2′-methyl-1′-[[1-(2-methylsulfonylethyl)triazol-4-yl]methyl]spiro[6,7-dihydrothieno[3,2-c]pyran-4,4′-piperidine](11)

Compound 11 was prepared from (2′S,4R)-2-ethyl-2′-methyl-1′-prop-2-ynyl-spiro[6,7-dihydrothieno[3,2-c]pyran-4,4′-piperidine] and 1-azido-2-methylsulfonyl-ethane using the same method as described for 10. The crude was suspended in EtOAc and heated at 50° C. for 10 min. The mixture was diluted with Et₂O and cooled to 0° C. and stirred for 10 min. The mixture was filtered and the cake was washed with Et₂O and dried to afford the title compound (595 mg, 70%). ¹H NMR (400 MHz, DMSO-d6) δ 8.08 (s, 1H), 6.58 (d, J=1.1 Hz, 1H), 4.80 (t, J=6.9 Hz, 2H), 3.91-3.65 (m, 6H), 2.92 (s, 3H), 2.70 (qd, J=7.5, 0.9 Hz, 2H), 2.65-2.53 (m, 3H), 2.48-2.36 (m, 2H), 1.82-1.62 (m, 3H), 1.54 (dd, J=13.6, 11.2 Hz, 1H), 1.19 (t, J=7.5 Hz, 3H), 1.13 (d, J=6.1 Hz, 3H). LCMS m/z 439.56 [M+H]⁺.

### Compound 12

### 2-[4-[[(2′S)-2-ethyl-2′-methyl-spiro[6,7-dihydrothieno[3,2-c]pyran-4,4′-piperidine]-1′-yl]methyl]triazol-1-yl]-N-methyl-ethanesulfonamide (12)

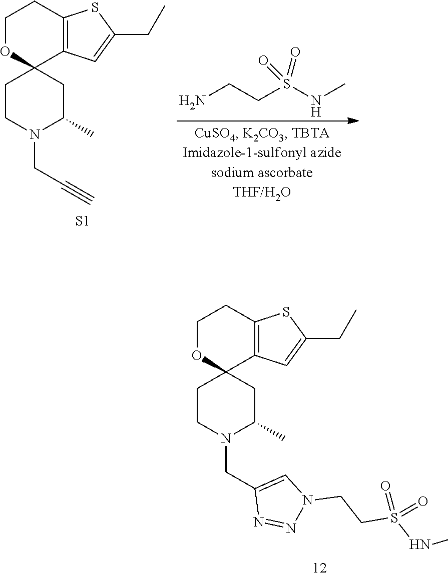

### Preparation of 2-[4-[[(2′S)-2-ethyl-2′-methyl-spiro[6,7-dihydrothieno[3,2-c]pyran-4,4′-piperidine]-1′-yl]methyl]triazol-1-yl]-N-methyl-ethanesulfonamide (12)

To a solution of 2-amino-N-methyl-ethanesulfonamide (HCl salt) (55 mg, 0.31 mmol) in THF (2.5 mL) and H₂O (1 mL) was added CuSO₄ (1 mg, 0.0063 mmol) and K₂CO₃ (30 mg, 0.2171 mmol) followed by N-diazoimidazole-1-sulfonamide (tetrafluoroborate) (120 mg, 0.46 mmol). The reaction was stirred at room temperature overnight. Subsequently TBTA (10 mg, 0.019 mmol), sodium ascorbate (40 mg, 0.23 mmol) and (2′S,4R)-2-ethyl-2′-methyl-1′-prop-2-ynyl-spiro[6,7-dihydrothieno[3,2-c]pyran-4,4′-piperidine]S1 (HCl salt) (56 mg, 0.17 mmol) were added to the reaction. The resulting mixture was stirred at 55° C. for 30 min. The reaction was cooled down to room temperature and the volatile was removed. The crude was purified by reversed phase chromatography (C18 column; Gradient: MeCN in H₂O with 0.1% TFA) to give the desired product as a TFA salt. The product was dissolved in DCM and treated with sat. NaHCO₃ solution. The organic layer was separated and concentrated in vacuo to afford 12 as a free base (53.5 mg, 70%. ¹H NMR (400 MHz, Chloroform-d) δ 7.54 (s, 1H), 6.38 (s, 1H), 4.74 (t, J=6.3 Hz, 2H), 4.63 (s, 1H), 3.93 (s, 2H), 3.86-3.69 (m, 2H), 3.60 (q, J=6.4 Hz, 2H), 2.76-2.58 (m, 5H), 2.56 (s, 3H), 2.52-2.37 (m, 2H), 1.89-1.71 (m, 3H), 1.61 (dd, J=13.8, 11.3 Hz, 1H), 1.24-1.10 (m, 6H). LCMS m/z 454.45 [M+H]⁺.

### Preparation S7

### azido(methylsulfonyl)methane (S7)

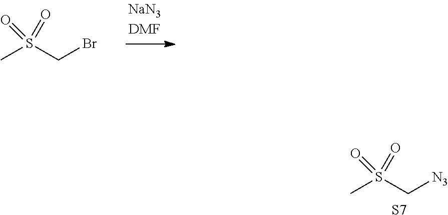

### Preparation of azido(methylsulfonyl)methane (S7)

To a solution of bromo(methylsulfonyl)methane (100 mg, 0.58 mmol) in DMF (3 mL) was added sodium azide (150 mg, 2.31 mmol). The resulting mixture was heated at 80° C. overnight. After cooling down to room temperature, the mixture was diluted with EtOAc and filtered. The filtrate was concentrated in vacuo. The crude was dissolved in DCM (5 mL) and filtered again. The filtrate was concentrated in vacuo to afford the title compound as a red oil (75 mg, 82%). The crude was used directly without further purification. ¹H NMR (400 MHz, Chloroform-d) δ 4.20 (s, 2H), 2.98 (t, J=0.9 Hz, 3H).

### Preparation S8

### 1-(azidomethylsulfonyl)ethane (S8)

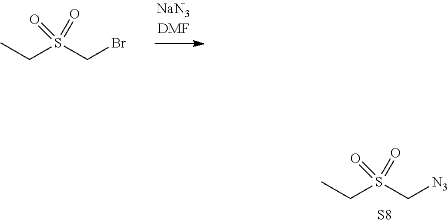

### Preparation of 1-(azidomethylsulfonyl)ethane (S8)

Compounds S8 was prepared from 1-(bromomethylsulfonyl)ethane using the same method as described for S7. The crude title compound was isolated as a red oil (300 mg, 88%) and used directly without further purification.

### Compound 13

### (2′S)-2-ethyl-2′-methyl-1′-[[1-(methylsulfonylmethyl)triazol-4-yl]methyl]spiro[6,7-dihydrothieno[3,2-c]pyran-4,4′-piperidine](13)

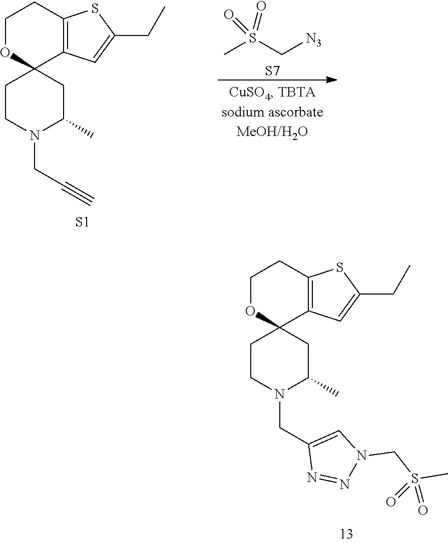

### Preparation of (2′S)-2-ethyl-2′-methyl-1′-[[1-(methylsulfonylmethyl)triazol-4-yl]methyl]spiro[6,7-dihydrothieno[3,2-c]pyran-4,4′-piperidine](13)

To a mixture of azido(methylsulfonyl)methane (75 mg, 0.47 mmol), (2′S,4R)-2-ethyl-2′-methyl-1′-prop-2-ynyl-spiro[6,7-dihydrothieno[3,2-c]pyran-4,4′-piperidine](50 mg, 0.16 mmol) in MeOH (3 mL) and H₂O (2 mL) was added CuSO₄ (0.6 mg, 0.004 mmol), TBTA (4 mg, 0.008 mmol) and sodium ascorbate (28 mg, 0.16 mmol). The resulting mixture was heated at 50° C. for 10 min. After cooling down to rt, the mixture was concentrated. The crude was purified using silica gel chromatography (0-10% MeOH in DCM) to afford the title compound (59 mg, 85%). ¹H NMR (400 MHz, Chloroform-d) δ 7.81 (s, 1H), 6.45 (d, J=1.1 Hz, 1H), 5.61-5.47 (m, 2H), 4.08 (d, J=14.7 Hz, 1H), 3.92-3.76 (m, 3H), 2.89 (d, J=0.9 Hz, 3H), 2.79-2.51 (m, 7H), 1.94-1.76 (m, 3H), 1.68 (dd, J=14.0, 11.3 Hz, 1H), 1.25 (t, J=7.5 Hz, 3H), 1.21 (d, J=6.2 Hz, 3H). LCMS m/z 425.52[M+H]⁺.

### Compound 14

### (2′S)-2-ethyl-1′-[[1-(ethylsulfonylmethyl)triazol-4-yl]methyl]-2′-methyl-spiro[6,7-dihydrothieno[3,2-c]pyran-4,4′-piperidine](14)

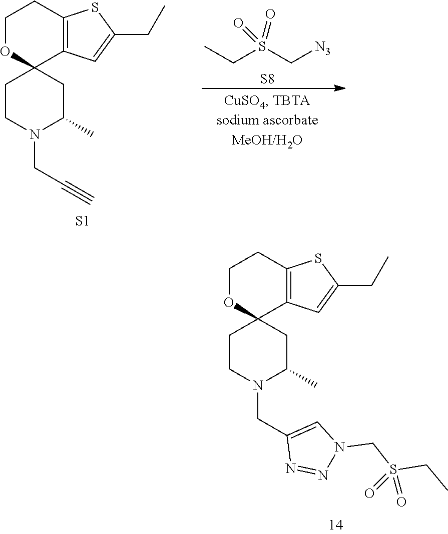

### Preparation of (2′S)-2-ethyl-1′-[[1-(ethylsulfonylmethyl)triazol-4-yl]methyl]-2′-methyl-spiro[6,7-dihydrothieno[3,2-c]pyran-4,4′-piperidine](14)

14 was prepared from (2′S,4R)-2-ethyl-2′-methyl-1′-prop-2-ynyl-spiro[6,7-dihydrothieno[3,2-c]pyran-4,4′-piperidine] and 1-(azidomethylsulfonyl)ethane using the same method as described for 13. The crude was purified using silica gel chromatography (0-100% EtOAc in heptane) to afford 14 as a clear oil (46 mg, 67%). ¹H NMR (400 MHz, Chloroform-d) δ 7.81 (s, 1H), 6.50-6.41 (m, 1H), 5.63-5.44 (m, 2H), 4.06 (d, J=14.7 Hz, 1H), 3.94-3.74 (m, 3H), 2.96 (q, J=7.5 Hz, 2H), 2.78-2.52 (m, 7H), 1.93-1.77 (m, 3H), 1.68 (dd, J=14.0, 11.3 Hz, 1H), 1.37 (t, J=7.5 Hz, 3H), 1.25 (t, J=7.5 Hz, 3H), 1.21 (d, J=6.2 Hz, 3H). LCMS m/z 439.56 [M+H]⁺.

### Compound 15

### tert-butyl (3R)-3-[4-[[(2′S,4R)-2-ethyl-2′-methyl-spiro[6,7-dihydrothieno[3,2-c]pyran-4,4′-piperidine]-1′-yl]methyl]triazol-1-yl]pyrrolidine-1-carboxylate (15)

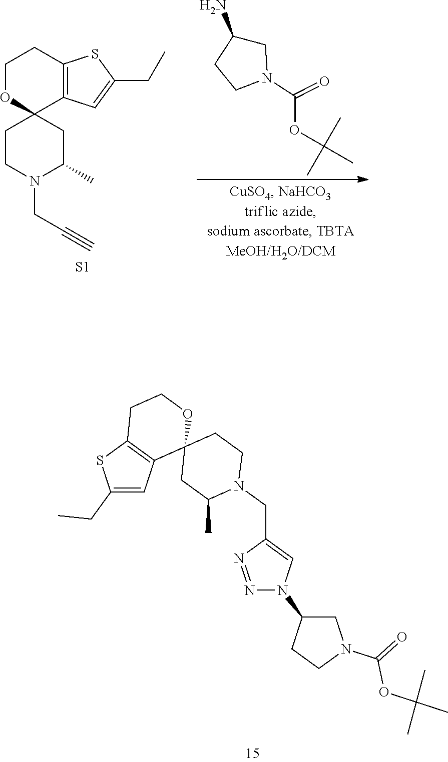

### Preparation of tert-butyl (3R)-3-[4-[[(2′S,4R)-2-ethyl-2′-methyl-spiro[6,7-dihydrothieno[3,2-c]pyran-4,4′-piperidine]-1′-yl]methyl]triazol-1-yl]pyrrolidine-1-carboxylate (15)

To a solution of tert-butyl (R)-3-aminopyrrolidine-1-carboxylate (9.1 μL, 0.054 mmol) in MeOH (600 μL) was added a solution of CuSO₄ (0.15 mg, 0.026 mmol) in H₂O (75 μL) and a solution of NaHCO₃ in H₂O (75 μL). A solution of triflic azide (0.15 mL, 0.45 mmol) in DCM was then added. After stirring the mixture at rt for 1 hour, a solution of (2′S,4R)-2-ethyl-2′-methyl-1′-prop-2-ynyl-spiro[6,7-dihydrothieno[3,2-c]pyran-4,4′-piperidine](10 mg, 0.036 mmol) in MeOH (100 μL) was added followed by a solution of sodium ascorbate (7 mg, 0.040 mmol) in H₂O (50 μL) and a solution of TBTA (2 mg, 0.004 mmol) in MeOH (100 μL). The resulting mixture was heated at 50° C. overnight. After cooling down to room temperature, the volatile was removed and the crude was purified by reversed phase chromatography (C18 column; Gradient: MeCN in H₂O with 0.1% TFA) to afford 15 (5.3 mg, 30%). ¹H NMR (300 MHz, Methanol-d4) δ 8.01 (s, 1H), 6.49 (d, J=1.1 Hz, 1H), 5.26 (tt, J=6.1, 4.2 Hz, 1H), 4.04 (d, J=14.5 Hz, 1H), 3.96-3.70 (m, 5H), 3.57 (d, J=7.9 Hz, 2H), 2.83-2.36 (m, 9H), 2.01-1.57 (m, 4H), 1.47 (s, 8H), 1.38-1.10 (m, 7H). LCMS m/z 502.38 [M+H]⁺.

### Compound 16

### 5-[4-[[(2′S,4R)-2-ethyl-2′-methyl-spiro[6,7-dihydrothieno[3,2-c]pyran-4,4′-piperidine]-1′-yl]methyl]triazol-1-yl]pyridin-3-ol (16)

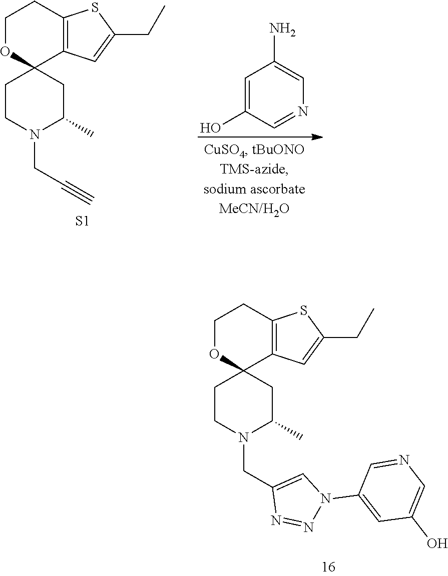

### Preparation of 5-[4-[[(2′S,4R)-2-ethyl-2′-methyl-spiro[6,7-dihydrothieno[3,2-c]pyran-4,4′-piperidine]-1′-yl]methyl]triazol-1-yl]pyridin-3-ol (16)

To a solution of 5-aminopyridin-3-ol (18 mg, 0.16 mmol) in MeCN (2 mL) was added dropwise tBuONO (0.28 mL, 0.24 mmol) and TMS azide (0.025 mL, 0.19 mmol). After stirring at rt for 2 h, (2′S,4R)-2-ethyl-2′-methyl-1′-prop-2-ynyl-spiro[6,7-dihydrothieno[3,2-c]pyran-4,4′-piperidine] (35 mg, 0.11 mmol) was added followed by an aqueous solution of CuSO₄ (1.2 mg, 0.008 mmol) and sodium ascorbate (30 mg, 0.17 mmol) in H₂O (0.2 mL). The resulting mixture was stirred at 65° C. overnight. After cooling down to rt, the crude was purified by reversed phase chromatography (C18 column; Gradient: MeCN in H₂O with 0.1% TFA) to give the desired product as a TFA salt. The product was dissolved in DCM (2 mL) and treated with a saturated aqueous solution of NaHCO₃ (2 mL). The organic layer was isolated and concentrated in vacuo to afford the title compound as a free base (15.4 mg, 33%). ¹H NMR (400 MHz, Chloroform-d) δ 8.44 (s, 1H), 8.36 (dd, J=10.0, 2.2 Hz, 2H), 8.10 (s, 1H), 6.40 (d, J=1.1 Hz, 1H), 4.22 (d, J=14.5 Hz, 1H), 3.97-3.71 (m, 3H), 2.94-2.53 (m, 8H), 1.98-1.62 (m, 4H), 1.27 (d, J=6.2 Hz, 3H), 1.19 (t, J=7.5 Hz, 3H). LCMS m/z 426.17[M+H]⁺.

### Compounds 17-197

Compounds 17-197 (see Table 3) were prepared from S1 using the appropriate amines employing general method B or C. All amines were obtained from commercial sources, unless noted otherwise. Any modifications to methods are noted in Table 2 and accompanying footnotes.

TABLE 3
Structure and physicochemical data for Compounds 17-197

| | | | Meth- | ¹H NMR; LCMS |
|---|---|---|---|---|
| Compd | Structure | Amine | od | m/z [M + H]⁺ |
| 17 | 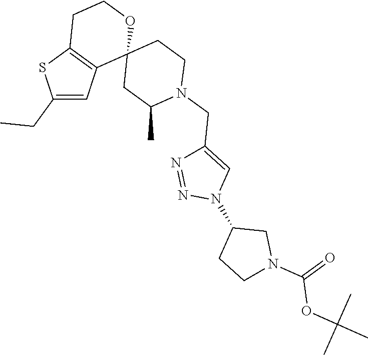 [SMILES: CCc1cc2c(s1)CCO[C@@]21CCN(Cc2cn([C@H]3CCN(C(=O)OC(C)(C)C)C3)nn2)[C@@H](C)C1] | 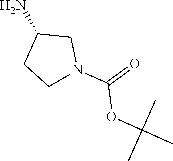 | Com- pound 15 | LCMS m/z [M + H]⁺ 502.38 |
| 18 | 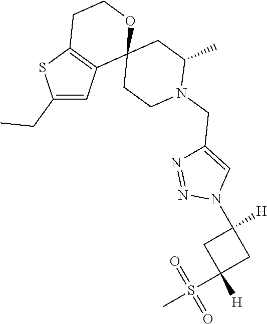 [SMILES: CCc1cc2c(s1)CCO[C@@]21CCN(Cc2cn([C@H]3C[C@H](S(C)(=O)=O)C3)nn2)[C@@H](C)C1] | 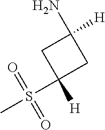 | Com- pound 15 | LCMS m/z [M + H]⁺ 465.19 |
| 19 | 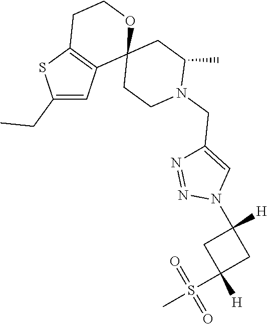 [SMILES: CCc1cc2c(s1)CCO[C@@]21CCN(Cc2cn([C@H]3C[C@@H](S(C)(=O)=O)C3)nn2)[C@@H](C)C1] | 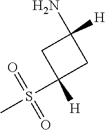 | Com- pound 15 | LCMS m/z [M + H]⁺ 465.2 |
| 20 | 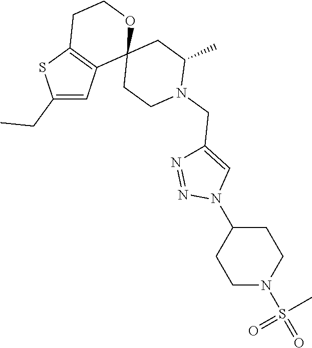 [SMILES: CCc1cc2c(s1)CCO[C@@]21CCN(Cc2cn(C3CCN(S(C)(=O)=O)CC3)nn2)[C@@H](C)C1] | 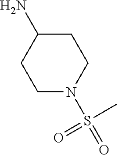 | Com- pound 15 | LCMS m/z [M + H]⁺ 494.05 |
| 21 | 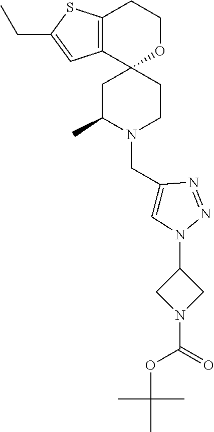 [SMILES: CCc1cc2c(s1)CCO[C@@]21CCN(Cc2cn(C3CN(C(=O)OC(C)(C)C)C3)nn2)[C@@H](C)C1] | 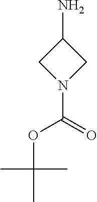 | Com- pound 15 | LCMS m/z [M + H]⁺ 488.07 |
| 22 | 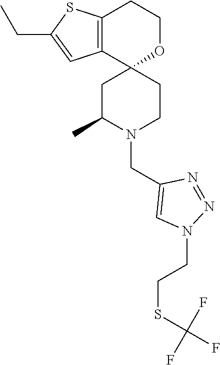 [SMILES: CCc1cc2c(s1)CCO[C@@]21CCN(Cc2cn(CCSC(F)(F)F)nn2)[C@@H](C)C1] | 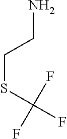 | Com- pound 15 | LCMS m/z [M + H]⁺ 461.19 |
| 23 | 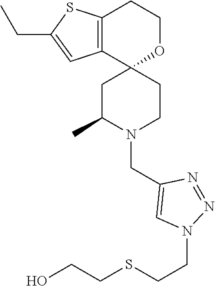 [SMILES: CCc1cc2c(s1)CCO[C@@]21CCN(Cc2cn(CCSCCO)nn2)[C@@H](C)C1] | 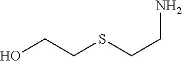 | Com- pound 15 | LCMS m/z [M + H]⁺ 437.21 |
| 24 | 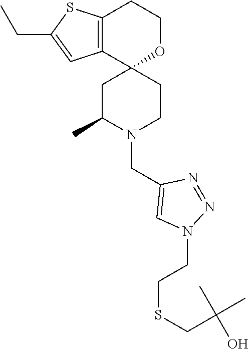 [SMILES: CCc1cc2c(s1)CCO[C@@]21CCN(Cc2cn(CCSCC(C)(C)O)nn2)[C@@H](C)C1] | 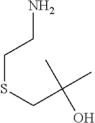 | Com- pound 15 | LCMS m/z [M + H]⁺ 465.26 |
| 25 | 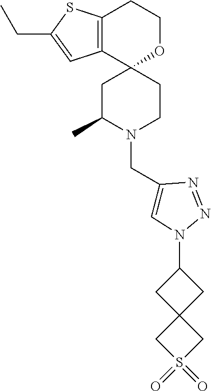 [SMILES: CCc1cc2c(s1)CCO[C@@]21CCN(Cc2cn(C3CC4(C3)CS(=O)(=O)C4)nn2)[C@@H](C)C1] | 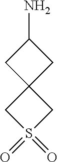 | Com- pound 15 | LCMS m/z [M + H]⁺ 477.35 |
| 26 | 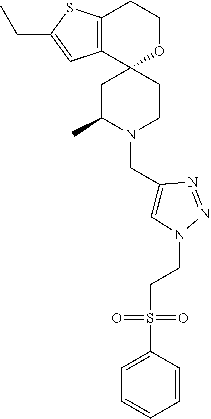 [SMILES: CCc1cc2c(s1)CCO[C@@]21CCN(Cc2cn(CCS(=O)(=O)c3ccccc3)nn2)[C@@H](C)C1] | 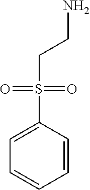 [SMILES: NCCS(=O)(=O)c1ccccc1] | Com- pound 15 | LCMS m/z [M + H]⁺ 501.22 |
| 27 | 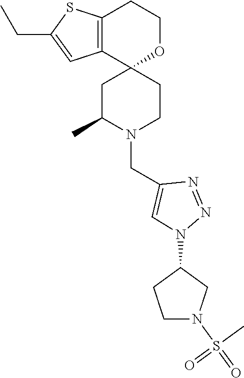 [SMILES: CCc1cc2c(s1)CCO[C@@]21CCN(Cc2cn([C@H]3CCN(S(C)(=O)=O)C3)nn2)[C@@H](C)C1] | 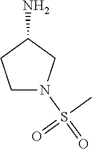 | Com- pound 15 | LCMS m/z [M + H]⁺ 480.23 |
| 28 | 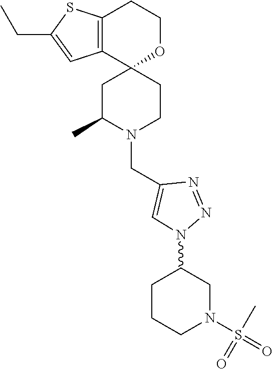 [SMILES: CCc1cc2c(s1)CCO[C@@]21CCN(Cc2cn(C3CCCN(S(C)(=O)=O)C3)nn2)[C@@H](C)C1] | 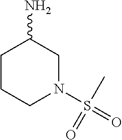 | Com- pound 15 | LCMS m/z [M + H]⁺ 494.28 |
| 29 | 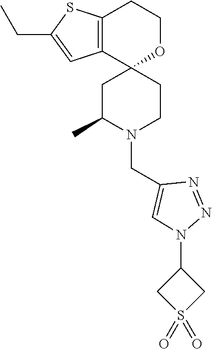 [SMILES: CCc1cc2c(s1)CCO[C@@]21CCN(Cc2cn(C3CS(=O)(=O)C3)nn2)[C@@H](C)C1] | 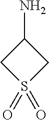 | Com- pound 15 | LCMS m/z [M + H]⁺ 437.2 |
| 30 | 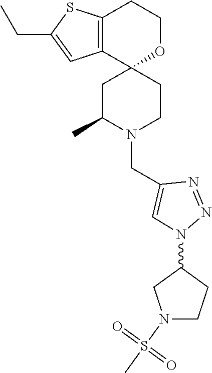 [SMILES: CCc1cc2c(s1)CCO[C@@]21CCN(Cc2cn(C3CCN(S(C)(=O)=O)C3)nn2)[C@@H](C)C1] | 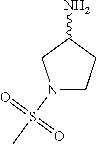 | Com- pound 15 | LCMS m/z [M + H]⁺ 480.19 |
| 31 | 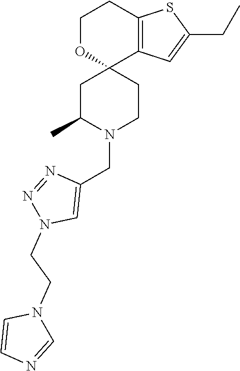 [SMILES: CCc1cc2c(s1)CCO[C@@]21CCN(Cc2cn(CCn3ccnc3)nn2)[C@@H](C)C1] | 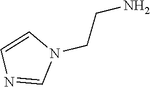 | Com- pound 15 | LCMS m/z [M + H]⁺ 427.01 |
| 32 | 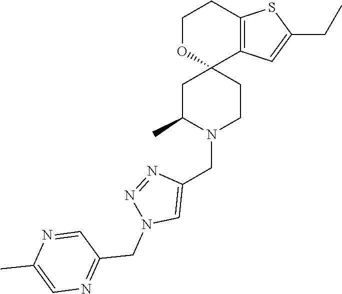 [SMILES: CCc1cc2c(s1)CCO[C@@]21CCN(Cc2cn(Cc3cnc(C)cn3)nn2)[C@@H](C)C1] | 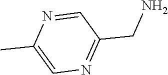 | Com- pound 15 | 1H NMR (400 MHz, DMSO-d6) δ 10.06 (s, 1H), 8.61 (s, 1H), 8.52 (s, 1H), 8.47 (s, 1H), 6.46 (s, 1H), 5.85 (s, 2H), 4.64 (d, J = 14.2 Hz, 1H), 4.53 (d, J = 14.5 Hz, 1H), 3.82 (q, J = 6.2 Hz, 3H), 3.32 (d, J = 11.9 Hz, 2H), 3.15 (s, 2H), 2.68 (s, 2H), 2.54 (s, 3H), 2.04 (t, J = 10.1 Hz, 4H), 1.44 (d, J = 6.4 Hz, 3H), 1.23-1.16 (m, 3H). |
| | | | | LCMS m/z [M + H]⁺ |
| | | | | 439.2 |
| 33 | 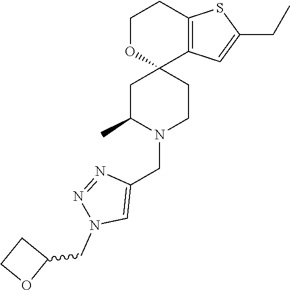 [SMILES: CCc1cc2c(s1)CCO[C@@]21CCN(Cc2cn(CC3CCO3)nn2)[C@@H](C)C1] | 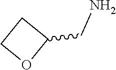 | Com- pound 15 | 1H NMR (400 MHz, DMSO-d6) δ 10.01 (s, 1H), 8.29 (s, 1H), 6.47 (d, J = 5.7 Hz, 1H), 4.76- 4.30 (m, 4H), 4.00 (s, 1H), 3.82 (s, 3H), 3.33 (s, 2H), 3.15 (s, 2H), 2.67 (s, 3H), 2.02 (d, J = 18.5 Hz, 4H), 1.75- 1.50 (m, 2H), 1.45 (d, J = 6.3 Hz, 3H), 1.25 (d, J = 6.5 Hz, 3H). Updated 1.75-1.50 peak LCMS m/z [M + H]⁺ 403.23 |
| 34 | 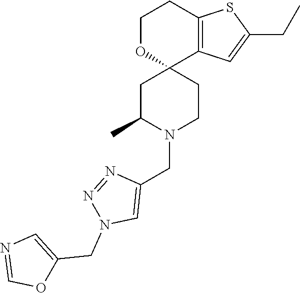 [SMILES: CCc1cc2c(s1)CCO[C@@]21CCN(Cc2cn(Cc3cnco3)nn2)[C@@H](C)C1] | 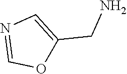 | Com- pound 15 | 1H NMR (400 MHz, DMSO-d6) δ 10.08 (s, 1H), 8.46- 8.34 (m, 1H), 8.38 (s, 1H), 7.35 (d, J = 3.5 Hz, 1H), 6.46 (s, 1H), 5.88 (d, J = 3.4 Hz, 2H), 4.63 (d, J = 13.9 Hz, 1H), 4.51 (d, J = 14.9 Hz, 1H), 3.82 (s, 2H), 3.63 (s, 1H), 3.32 (s, 1H), 3.15 (s, 2H), 2.68 (s, 2H), 2.03 (s, 4H), 1.44 (d, J = 7.0 Hz, 3H), 1.20 (q, J = 7.8, 6.9 Hz, |
| | | | | 3H). LCMS m/z |
| | | | | [M + H]⁺ 414.21 |
| 35 | 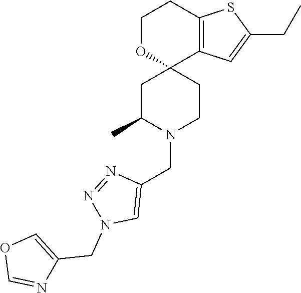 [SMILES: CCc1cc2c(s1)CCO[C@@]21CCN(Cc2cn(Cc3cocn3)nn2)[C@@H](C)C1] | 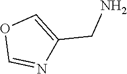 | Com- pound 15 | 1H NMR (400 MHz, DMSO-d6) δ 10.06 (s, 1H), 8.44 (d, J = 3.7 Hz, 1H), 8.38 (d, J = 3.5 Hz, 1H), 8.26 (d, J = 3.6 Hz, 1H), 6.46 (s, 1H), 5.64 (d, J = 3.5 Hz, 2H), 4.63 (d, J = 14.2 Hz, 1H), 4.51 (d, J = 14.8 Hz, 1H), 3.82 (s, 2H), 3.62 (s, 1H), 3.32 (s, 2H), 3.15 (s, 2H), 2.68 (s, 2H), 2.02 (d, J = 13.2 Hz, 4H), 1.44 (d, J = 6.1 Hz, 3H), |
| | | | | 1.23-1.12 (m, 3H). |
| | | | | LCMS m/z [M + H]⁺ |
| | | | | 414.21 |
| 36 | 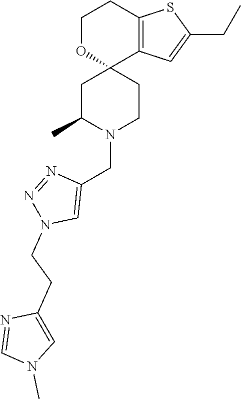 [SMILES: CCc1cc2c(s1)CCO[C@@]21CCN(Cc2cn(CCc3cn(C)cn3)nn2)[C@@H](C)C1] | 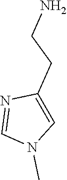 | Com- pound 15 | 1H NMR (400 MHz, DMSO-d6) δ 8.84 (s, 1H), 8.33 (d, J = 3.3 Hz, 1H), 7.30 (d, J = 12.3 Hz, 1H), 6.49 (s, 1H), 4.75 (s, 2H), 4.58 (s, 1H), 4.51 (d, J = 13.8 Hz, 1H), 3.83 (s, 2H), 3.77 (d, J = 3.3 Hz, 3H), 3.63 (s, 1H), 3.27 (s, 4H), 3.13 (s, 2H), 2.69 (s, 2H), 2.05 (s, 4H), 1.43 (s, 3H), 1.21 (d, J = 10.1 Hz, 3H). LCMS m/z [M + H]⁺ 441.22 |
| 37 | 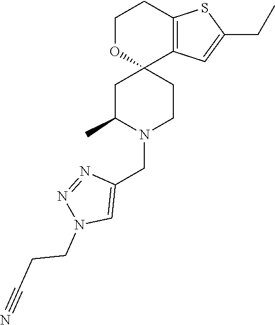 [SMILES: CCc1cc2c(s1)CCO[C@@]21CCN(Cc2cn(CCC#N)nn2)[C@@H](C)C1] | 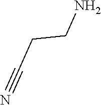 | Com- pound 15 | 1H NMR (400 MHz, DMSO-d6) δ 10.03 (s, 1H), 8.45 (d, J = 3.1 Hz, 1H), 6.47 (s, 1H), 4.81- 4.70 (m, 2H), 4.67 (d, J = 14.0 Hz, 1H), 4.56 (d, J = 14.6 Hz, 1H), 3.81 (s, 2H), 3.34-3.03 (m, 7H), 2.68 (s, 2H), 2.03 (s, 4H), 1.45 (d, J = 6.2 Hz, 3H), 1.21 (d, J = 9.4 Hz, 3H). LCMS m/z [M + H]⁺ 386.2 |
| 38 | 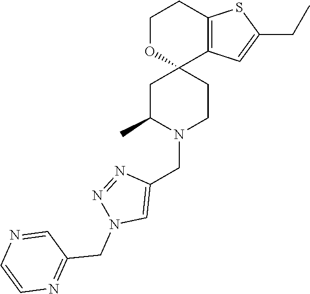 [SMILES: CCc1cc2c(s1)CCO[C@@]21CCN(Cc2cn(Cc3cnccn3)nn2)[C@@H](C)C1] | 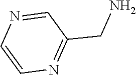 | Com- pound 15 | 1H NMR (400 MHz, DMSO-d6) δ 10.08 (s, 1H), 8.73 (s, 1H), 8.69-8.59 (m, 2H), 8.50 (s, 1H), 6.47 (s, 1H), 5.92 (s, 2H), 4.65 (d, J = 14.3 Hz, 1H), 4.55 (d, J = 14.3 Hz, 1H), 3.81 (dd, J = 12.6, 6.5 Hz, 2H), 3.63 (s, 1H), 3.33 (d, J = 12.6 Hz, 2H), 3.24- 3.07 (m, 2H), 2.68 (s, 2H), 2.03 (q, J = 17.4, 14.4 Hz, 4H), 1.45 (d, J = 6.4 Hz, |
| | | | | 3H), 1.20 (t, J = 7.6 |
| | | | | Hz, 3H). LCMS |
| | | | | m/z [M + H]⁺ 425.06 |
| 39 | 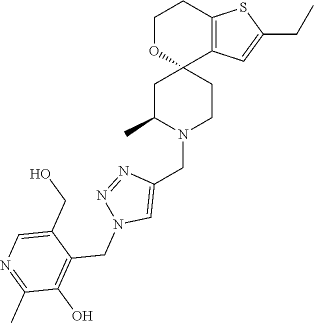 [SMILES: CCc1cc2c(s1)CCO[C@@]21CCN(Cc2cn(Cc3c(CO)cnc(C)c3O)nn2)[C@@H](C)C1] | 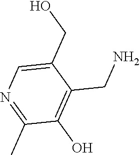 | Com- pound 15 | 1H NMR (400 MHz, DMSO-d6) δ 10.09 (s, 1H), 8.97 (s, 2H), 8.28 (s, 1H), 8.14 (s, 1H), 6.46 (s, 1H), 5.77 (s, 2H), 4.67 (s, 2H), 4.60 (d, J = 14.3 Hz, 1H), 4.47 (d, J = 14.3 Hz, 1H), 3.84-3.80 (m, 4H), 3.62 (d, J = 3.9 Hz, 2H), 3.30 (s, 2H), 3.14 (dd, J = 7.3, 4.1 Hz, 2H), 2.67 (s, 2H), 1.99 (dd, J = 21.8, 10.8 Hz, 4H), 1.42 (d, J = 6.3 Hz, 3H), 1.19 (t, J = 7.4 Hz, 3H). |
| | | | | LCMS m/z [M + H]⁺ |
| | | | | 484.35 |
| 40 | 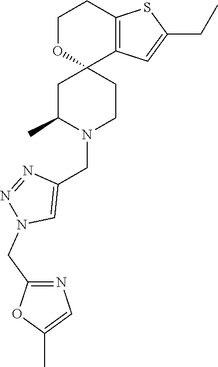 [SMILES: CCc1cc2c(s1)CCO[C@@]21CCN(Cc2cn(Cc3ncc(C)o3)nn2)[C@@H](C)C1] | 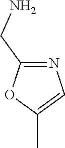 | Com- pound 15 | 1H NMR (400 MHz, DMSO-d6) δ 10.03 (s, 1H), 8.48 (s, 1H), 6.88 (s, 1H), 6.46 (s, 1H), 5.88 (s, 2H), 4.66 (d, J = 14.3 Hz, 1H), 4.54 (d, J = 14.3 Hz, 1H), 3.82 (td, J = 12.1, 5.9 Hz, 2H), 3.72- 3.49 (m, 1H), 3.30 (s, 1H), 3.22-3.04 (m, 2H), 2.68 (d, J = 5.8 Hz, 2H), 2.28 (s, 3H), 2.03 (d, J = 10.6 Hz, 4H), 1.45 (d, J = 6.4 Hz, 3H), 1.20 (t, J = 7.6 Hz, 4H). LCMS m/z [M + H]⁺ 428.22 |
| 41 | 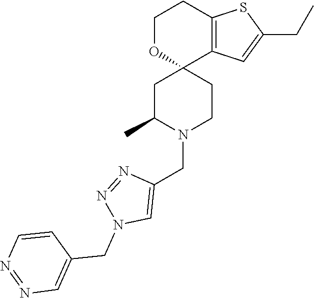 [SMILES: CCc1cc2c(s1)CCO[C@@]21CCN(Cc2cn(Cc3ccnnc3)nn2)[C@@H](C)C1] | 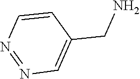 | Com- pound 15 | 1H NMR (400 MHz, DMSO-d6) δ 10.10 (s, 1H), 9.26 (d, J = 5.3 Hz, 1H), 9.16 (d, J = 2.1 Hz, 1H), 8.54 (s, 1H), 7.58-7.44 (m, 1H), 6.47 (s, 1H), 5.86 (s, 2H), 4.66 (d, J = 14.2 Hz, 1H), 4.56 (d, J = 14.3 Hz, 1H), 3.83 (tt, J = 11.6, 5.8 Hz, 3H), 3.34 (d, J = 12.2 Hz, 2H), 3.25- 3.03 (m, 2H), 2.68 (s, 2H), 2.14-1.82 (m, 4H), 1.45 (d, |
| | | | | J = 6.3 Hz, 3H), 1.19 |
| | | | | (d, J = 7.7 Hz, 3H). |
| | | | | LCMS m/z [M + H]⁺ |
| | | | | 425.22 |
| 42 | 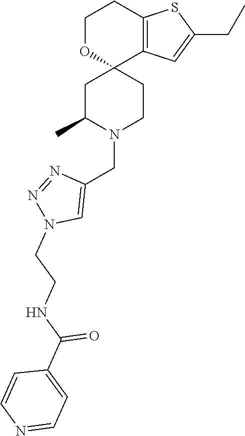 [SMILES: CCc1cc2c(s1)CCO[C@@]21CCN(Cc2cn(CCNC(=O)c3ccncc3)nn2)[C@@H](C)C1] | 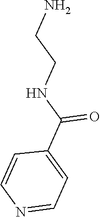 [SMILES: NCCNC(=O)c1ccncc1] | Com- pound 15 | 1H NMR (400 MHz, DMSO-d6) δ 9.98 (s, 1H), 8.97 (s, 1H), 8.72 (d, J = 5.2 Hz, 2H), 8.36 (s, 1H), 7.67 (d, J = 5.4 Hz, 2H), 6.45 (s, 1H), 4.75-4.39 (m, 4H), 3.77 (d, J = 5.7 Hz, 5H), 3.37- 3.19 (m, 2H), 3.18- 3.03 (m, 2H), 2.66 (s, 2H), 1.95 (dd, J = 39.0, 11.3 Hz, 4H), 1.40 (d, J = 6.4 Hz, 3H), 1.20 (t, J = 7.5 Hz, 3H). LCMS m/z [M + H]⁺ 481.19 |
| 43 | 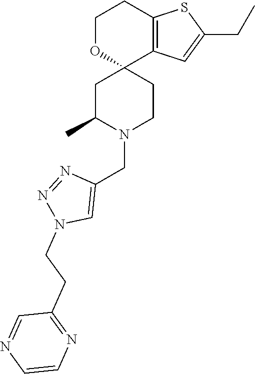 [SMILES: CCc1cc2c(s1)CCO[C@@]21CCN(Cc2cn(CCc3cnccn3)nn2)[C@@H](C)C1] | 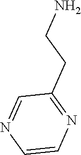 | Com- pound 15 | 1H NMR (400 MHz, DMSO-d6) δ 10.02 (s, 1H), 8.55 (t, J = 2.0 Hz, 1H), 8.51 (d, J = 2.0 Hz, 2H), 8.31 (s, 1H), 6.46 (s, 1H), 4.88 (t, J = 6.8 Hz, 2H), 4.57 (d, J = 14.1 Hz, 1H), 4.48 (d, J = 13.8 Hz, 1H), 3.83 (h, J = 6.2 Hz, 2H), 3.43 (t, J = 6.8 Hz, 2H), 3.29- 3.02 (m, 4H), 2.68 (s, 2H), 2.21-1.83 (m, 4H), 1.41 (d, J = 6.4 Hz, 3H), 1.23-1.12 (m, 3H). LCMS m/z [M + H]⁺ 439.07 |
| 44 | 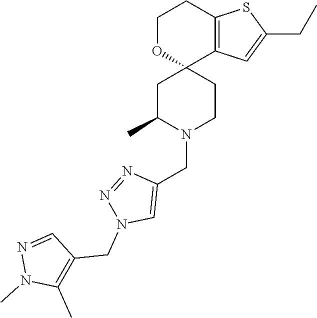 [SMILES: CCc1cc2c(s1)CCO[C@@]21CCN(Cc2cn(Cc3cnn(C)c3C)nn2)[C@@H](C)C1] | 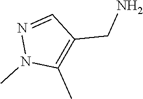 | Com- pound 15 | 1H NMR (400 MHz, DMSO-d6) δ 10.00 (s, 1H), 8.28 (d, J = 3.4 Hz, 1H), 7.46-7.36 (m, 1H), 6.46 (s, 1H), 5.48 (d, J = 3.5 Hz, 2H), 4.59 (d, J = 14.2 Hz, 1H), 4.47 (d, J = 14.7 Hz, 1H), 3.82 (s, 2H), 3.71 (d, J = 3.6 Hz, 3H), 3.62 (s, 1H), 3.30 (d, J = 11.9 Hz, 2H), 3.14 (s, 2H), 2.68 (s, 2H), 2.26 (d, J = 3.5 Hz, 3H), 2.02 (d, J = 10.2 Hz, 4H), 1.43 (d, J = 6.3 Hz, 3H), 1.20 |
| | | | | (q, J = 7.3 Hz, 3H). |
| | | | | LCMS m/z [M + H]⁺ |
| | | | | 441.22 |
| 45 | 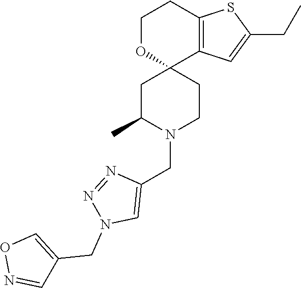 [SMILES: CCc1cc2c(s1)CCO[C@@]21CCN(Cc2cn(Cc3cnoc3)nn2)[C@@H](C)C1] | 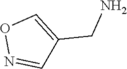 | Com- pound 15 | 1H NMR (400 MHz, DMSO-d6) δ 10.05 (s, 1H), 9.10 (s, 1H), 8.72 (s, 1H), 8.42 (s, 1H), 6.46 (s, 1H), 5.64 (s, 2H), 4.62 (d, J = 14.2 Hz, 1H), 4.50 (d, J = 14.4 Hz, 1H), 3.98-3.71 (m, 2H), 3.62 (d, J = 5.4 Hz, 1H), 3.29 (s, 2H), 3.14 (dd, J = 7.4, 4.1 Hz, 2H), 2.67 (t, J = 5.5 Hz, 2H), 2.02 (d, J = 10.1 Hz, 4H), 1.43 (d, J = 6.4 Hz, 3H), |
| | | | | 1.19 (t, J = 7.5 Hz, |
| | | | | 3H). LCMS m/z |
| | | | | [M + H]⁺ 414.18 |
| 46 | 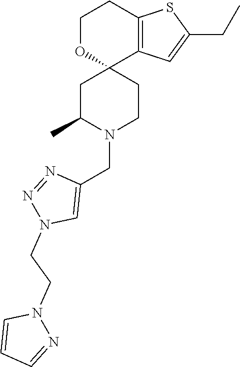 [SMILES: CCc1cc2c(s1)CCO[C@@]21CCN(Cc2cn(CCn3cccn3)nn2)[C@@H](C)C1] | 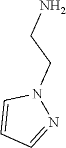 | Com- pound 15 | 1H NMR (400 MHz, DMSO-d6) δ 10.05 (s, 1H), 7.96 (s, 1H), 7.49 (d, J = 2.2 Hz, 1H), 7.43 (d, J = 1.9 Hz, 1H), 6.47 (s, 1H), 6.17 (t, J = 2.1 Hz, 1H), 4.88 (t, J = 5.7 Hz, 2H), 4.64 (t, J = 5.9 Hz, 2H), 4.61- 4.45 (m, 2H), 3.95- 3.74 (m, 2H), 3.62 (s, 1H), 3.13 (dd, J = 41.5, 11.9 Hz, 4H), 2.69 (d, J = 5.6 Hz, 2H), 2.03 (p, J = 14.8, 14.3 Hz, 4H), 1.40 (d, J = 6.4 Hz, 3H), 1.23-1.15 (m, 3H). LCMS m/z [M + H]⁺ 427.24 |
| 47 | 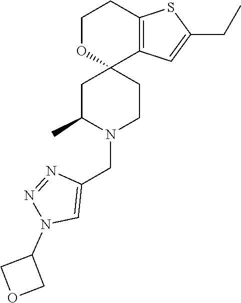 [SMILES: CCc1cc2c(s1)CCO[C@@]21CCN(Cc2cn(C3COC3)nn2)[C@@H](C)C1] | 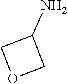 | Com- pound 15 | 1H NMR (400 MHz, DMSO-d6) δ 9.98 (s, 1H), 8.35 (s, 1H), 6.46 (s, 1H), 5.05 (t, J = 7.3 Hz, 1H), 4.92 (q, J = 6.0 Hz, 1H), 4.71- 4.42 (m, 3H), 3.82 (d, J = 6.0 Hz, 3H), 3.62 (s, 1H), 3.33- 3.02 (m, 4H), 2.68 (s, 2H), 2.04 (d, J = 7.8 Hz, 4H), 1.45 (dd, J = 6.5, 2.5 Hz, 3H), 1.20 (t, J = 7.6 Hz, 3H). LCMS m/z [M + H]⁺ 389.19 |
| 48 | 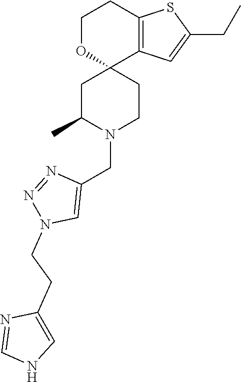 [SMILES: CCc1cc2c(s1)CCO[C@@]21CCN(Cc2cn(CCc3c[nH]cn3)nn2)[C@@H](C)C1] | 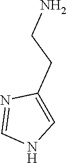 | Com- pound 15 | 1H NMR (400 MHz, DMSO-d6) δ 8.94 (s, 2H), 8.31 (s, 1H), 7.31 (s, 1H), 6.48 (s, 1H), 4.78 (t, J = 6.8 Hz, 2H), 4.59 (d, J = 14.6 Hz, 1H), 4.49 (d, J = 14.4 Hz, 1H), 3.82 (q, J = 6.3 Hz, 2H), 3.62 (s, 1H), 3.29 (t, J = 6.8 Hz, 4H), 3.18- 3.05 (m, 2H), 2.70 (d, J = 11.0 Hz, 2H), 2.01 (q, J = 19.1, 14.3 Hz, 4H), 1.42 (d, J = 6.4 Hz, 3H), 1.20 (t, J = 7.5 Hz, 3H). LCMS m/z [M + H]⁺ 427.34 |
| 49 | 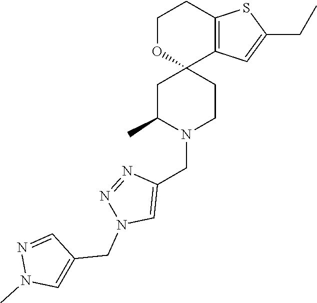 [SMILES: CCc1cc2c(s1)CCO[C@@]21CCN(Cc2cn(Cc3cnn(C)c3)nn2)[C@@H](C)C1] | 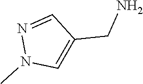 | Com- pound 15 | 1H NMR (400 MHz, DMSO-d6) δ 10.04 (s, 1H), 8.32 (s, 1H), 7.81 (s, 1H), 7.48 (s, 1H), 6.46 (s, 1H), 5.52 (s, 2H), 4.59 (d, J = 14.2 Hz, 1H), 4.48 (d, J = 14.6 Hz, 1H), 3.81 (s, 5H), 3.63 (s, 1H), 3.30 (d, J = 11.9 Hz, 2H), 3.21-3.04 (m, 2H), 2.67 (s, 2H), 2.02 (d, J = 9.8 Hz, 4H), 1.43 (d, J = 6.4 Hz, 3H), 1.19 (t, J = 7.4 Hz, 3H). LCMS m/z [M + H]⁺ |
| | | | | 427.21 |
| 50 | 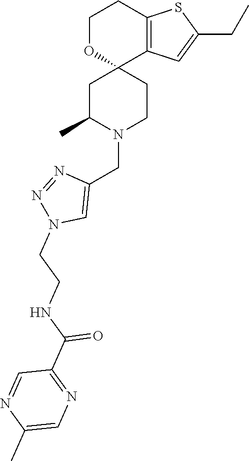 [SMILES: CCc1cc2c(s1)CCO[C@@]21CCN(Cc2cn(CCNC(=O)c3cnc(C)cn3)nn2)[C@@H](C)C1] | 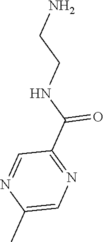 | Com- pound 15 | 1H NMR (400 MHz, DMSO-d6) δ 9.93 (s, 1H), 9.07 (t, J = 6.2 Hz, 1H), 8.96 (s, 1H), 8.62 (s, 1H), 8.32 (s, 1H), 6.45 (s, 1H), 4.65 (t, J = 5.8 Hz, 2H), 4.58 (d, J = 14.3 Hz, 1H), 4.47 (d, J = 14.2 Hz, 1H), 3.78 (d, J = 6.5 Hz, 4H), 3.62 (s, 1H), 3.21 (s, 2H), 3.13 (d, J = 14.4 Hz, 2H), 2.67 (s, 2H), 2.57 (s, 3H), 1.96 (dd, J = 29.3, 11.3 Hz, 4H), 1.39 (d, J = 6.3 Hz, 3H), 1.22-1.17 (m, 3H). LCMS m/z [M + H]⁺ 496.17 |
| 51 | 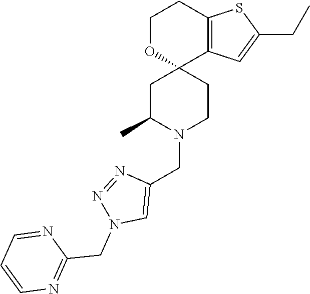 [SMILES: CCc1cc2c(s1)CCO[C@@]21CCN(Cc2cn(Cc3ncccn3)nn2)[C@@H](C)C1] | 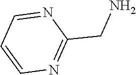 | Com- pound 15 | 1H NMR (400 MHz, DMSO-d6) δ 10.09 (s, 1H), 8.81 (s, 1H), 8.80 (s, 1H), 8.49 (s, 1H), 7.50 (t, J = 4.9 Hz, 1H), 6.47 (s, 1H), 5.96 (s, 2H), 4.68 (d, J = 14.2 Hz, 1H), 4.59 (d, J = 12.3 Hz, 1H), 3.83 (q, J = 6.0 Hz, 2H), 3.63 (d, J = 6.6 Hz, 1H), 3.34 (d, J = 11.8 Hz, 2H), 3.19 (d, J = 12.3 Hz, 2H), 2.67 (d, J = 5.6 Hz, 2H), 2.06 |
| | | | | (t, J = 11.3 Hz, |
| | | | | 4H), 1.46 (d, J = |
| | | | | 6.4 Hz, 3H), 1.20 |
| | | | | (t, J = 7.5 Hz, 3H). |
| | | | | LCMS m/z [M + H]⁺ |
| | | | | 425.22 |
| 52 | 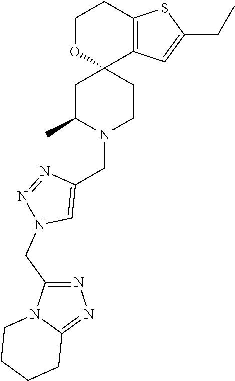 [SMILES: CCc1cc2c(s1)CCO[C@@]21CCN(Cc2cn(Cc3nnc4n3CCCC4)nn2)[C@@H](C)C1] | 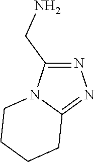 [SMILES: NCc1nnc2n1CCCC2] | Com- pound 15 | 1H NMR (400 MHz, DMSO-d6) δ 10.13 (s, 1H), 8.46 (s, 1H), 6.46 (s, 1H), 5.94 (s, 2H), 4.64 (d, J = 14.3 Hz, 1H), 4.53 (d, J = 14.2 Hz, 1H), 3.95 (d, J = 6.2 Hz, 2H), 3.81 (p, J = 5.6 Hz, 3H), 3.32 (d, J = 11.9 Hz, 2H), 3.00 (s, 2H), 2.86 (t, J = 6.4 Hz, 2H), 2.67 (s, 2H), 2.06 (q, J = 16.0, 14.8 Hz, 4H), 1.91 (d, J = 5.9 Hz, 2H), 1.81 (d, J = 6.5 Hz, 2H), 1.44 (d, J = 6.3 Hz, 3H), 1.19 (t, J = 7.5 Hz, 3H). LCMS m/z [M + H]⁺ 468.25 |
| 53 | 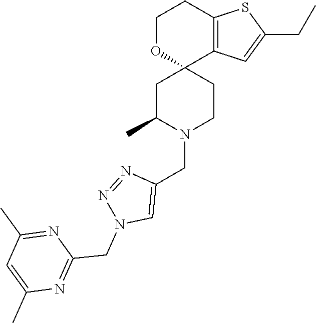 [SMILES: CCc1cc2c(s1)CCO[C@@]21CCN(Cc2cn(Cc3nc(C)cc(C)n3)nn2)[C@@H](C)C1] | 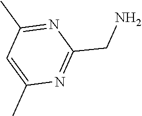 | Com- pound 15 | 1H NMR (400 MHz, DMSO-d6) δ 10.08 (s, 1H), 8.45 (s, 1H), 7.25 (d, J = 14.4 Hz, 1H), 6.47 (s, 1H), 5.84 (s, 2H), 4.68 (d, J = 14.3 Hz, 1H), 4.59 (d, J = 14.4 Hz, 1H), 3.81 (q, J = 5.9 Hz, 3H), 3.32 (d, J = 11.7 Hz, 2H), 3.24-3.04 (m, 2H), 2.67 (s, 2H), 2.37 (s, 6H), 2.06 (t, J = 12.0 Hz, 4H), 1.46 (d, J = 6.3 Hz, 3H), 1.19 (d, J = 7.4 Hz, 3H). LCMS m/z [M + H]⁺ |
| | | | | 453.2 |
| 54 | 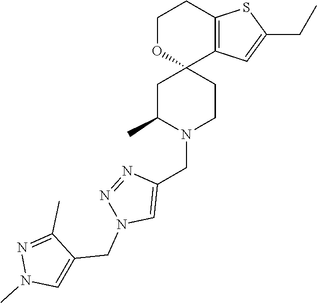 [SMILES: CCc1cc2c(s1)CCO[C@@]21CCN(Cc2cn(Cc3cn(C)nc3C)nn2)[C@@H](C)C1] | 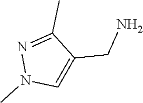 | Com- pound 15 | 1H NMR (400 MHz, DMSO-d6) δ 10.04 (s, 1H), 8.25 (s, 1H), 7.72 (s, 1H), 6.46 (s, 1H), 5.47 (s, 2H), 4.58 (d, J = 14.3 Hz, 1H), 4.48 (d, J = 12.3 Hz, 1H), 3.81 (h, J = 5.8 Hz, 3H), 3.73 (s, 3H), 3.30 (d, J = 11.9 Hz, 2H), 3.13 (s, 2H), 2.66 (d, J = 5.7 Hz, 2H), 2.09 (s, 3H), 2.01 (q, J = 16.9, 13.8 Hz, 4H), 1.43 (d, J = 6.3 Hz, 3H), 1.19 (t, J = 7.4 Hz, |
| | | | | 3H). LCMS m/z |
| | | | | [M + H]⁺ 441.25 |
| 55 | 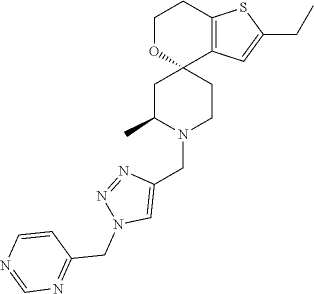 [SMILES: CCc1cc2c(s1)CCO[C@@]21CCN(Cc2cn(Cc3ccncn3)nn2)[C@@H](C)C1] | 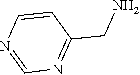 | Com- pound 15 | 1H NMR (400 MHz, DMSO-d6) δ 10.09 (s, 1H), 9.14 (d, J = 1.4 Hz, 1H), 8.85 (d, J = 5.2 Hz, 1H), 8.50 (s, 1H), 7.41 (d, J = 5.2 Hz, 1H), 6.47 (s, 1H), 5.91 (s, 2H), 4.68 (d, J = 14.3 Hz, 1H), 4.58 (d, J = 14.4 Hz, 1H), 3.83 (q, J = 6.1 Hz, 2H), 3.62 (s, 1H), 3.34 (d, J = 12.0 Hz, 2H), 3.26-3.07 (m, 2H), 2.67 (d, J = 5.5 Hz, 2H), 2.05 |
| | | | | (t, J = 10.5 Hz, |
| | | | | 4H), 1.46 (d, J = |
| | | | | 6.4 Hz, 3H), 1.19 |
| | | | | (d, J = 7.3 Hz, 3H). |
| | | | | LCMS m/z [M + H]⁺ |
| | | | | 425.22 |
| 56 | 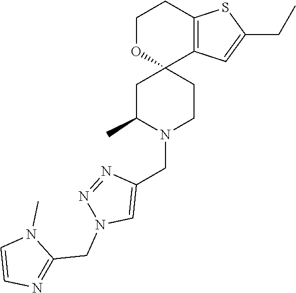 [SMILES: CCc1cc2c(s1)CCO[C@@]21CCN(Cc2cn(Cc3nccn3C)nn2)[C@@H](C)C1] | 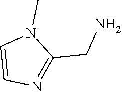 | Com- pound 15 | 1H NMR (400 MHz, DMSO-d6) δ 10.21 (s, 1H), 8.97 (s, 1H), 8.47 (s, 1H), 7.53 (s, 1H), 7.37 (s, 1H), 6.47 (s, 1H), 6.01 (s, 2H), 4.64 (d, J = 14.4 Hz, 1H), 4.54 (d, J = 14.4 Hz, 1H), 3.80 (s, 4H), 3.65-3.60 (m, 1H), 3.32 (d, J = 11.5 Hz, 2H), 3.15 (d, J = 7.5 Hz, 2H), 2.68 (s, 2H), 2.01 (dd, J = 24.3, 11.1 Hz, 4H), 1.44 (d, J = |
| | | | | 6.3 Hz, 3H), 1.19 |
| | | | | (q, J = 7.6 Hz, 3H). |
| | | | | LCMS m/z [M + H]⁺ |
| | | | | 427.21 |
| 57 | 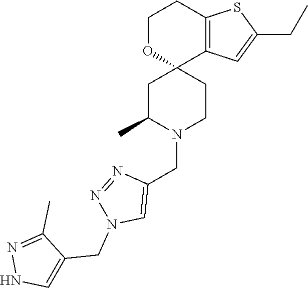 [SMILES: CCc1cc2c(s1)CCO[C@@]21CCN(Cc2cn(Cc3c[nH]nc3C)nn2)[C@@H](C)C1] | 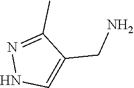 | Com- pound 15 | 1H NMR (400 MHz, DMSO-d6) δ 10.03 (s, 1H), 8.26 (s, 1H), 7.60 (s, 1H), 6.46 (s, 1H), 5.49 (s, 2H), 4.59 (d, J = 14.3 Hz, 1H), 4.48 (d, J = 14.3 Hz, 1H), 3.83- 3.79 (m, 2H), 3.65- 3.60 (m, 1H), 3.28 (s, 2H), 3.21-3.07 (m, 2H), 2.67 (s, 2H), 2.18 (s, 3H), 2.01 (q, J = 17.1, 14.2 Hz, 4H), 1.43 (d, J = 6.3 Hz, 3H), 1.19 (t, J = 7.5 Hz, |
| | | | | 3H). LCMS m/z |
| | | | | [M + H]⁺ 427.24 |
| 58 | 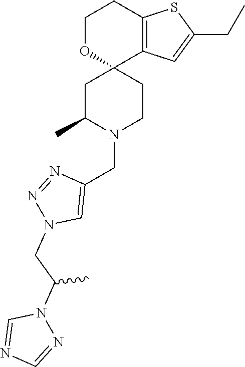 [SMILES: CCc1cc2c(s1)CCO[C@@]21CCN(Cc2cn(CC(C)n3cncn3)nn2)[C@@H](C)C1] | 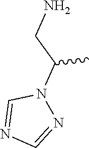 | Com- pound 15 | 1H NMR (400 MHz, DMSO-d6) δ 10.06 (s, 1H), 8.32 (d, J = 12.2 Hz, 1H), 8.03 (d, J = 11.6 Hz, 1H), 7.96 (d, J = 3.8 Hz, 1H), 6.46 (s, 1H), 5.09 (s, 1H), 4.91 (dt, J = 13.8, 4.5 Hz, 1H), 4.82 (td, J = 8.9, 4.5 Hz, 1H), 4.54 (d, J = 18.5 Hz, 2H), 3.86 (q, J = 9.1, 6.8 Hz, 3H), 3.12 (d, J = 98.3 Hz, 4H), 2.68 (s, 2H), 2.13-1.90 (m, 4H), 1.55 (dd, J = 6.9, 2.4 Hz, 3H), 1.40 (dd, J = 11.8, 6.3 Hz, 3H), 1.20 (t, J = 7.6 Hz, 3H). |
| | | | | LCMS m/z [M + H]⁺ |
| | | | | 442.19 |
| 59 | 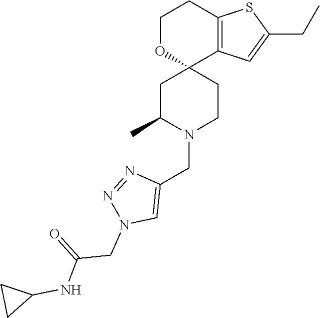 [SMILES: CCc1cc2c(s1)CCO[C@@]21CCN(Cc2cn(CC(=O)NC3CC3)nn2)[C@@H](C)C1] | 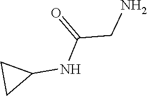 | Com- pound 15 | 1H NMR (400 MHz, DMSO-d6) δ 8.97 (s, 1H), 8.54 (d, J = 4.1 Hz, 1H), 8.33 (s, 1H), 6.47 (s, 1H), 5.13 (s, 2H), 4.65 (d, J = 14.3 Hz, 1H), 4.55 (d, J = 14.5 Hz, 1H), 3.82 (q, J = 6.2 Hz, 2H), 3.22- 3.06 (m, 2H), 2.66 (d, J = 7.2 Hz, 3H), 2.05 (t, J = 10.5 Hz, 4H), 1.45 (d, J = 6.3 Hz, 3H), 1.32 (d, J = 40.1 Hz, 2H), 1.19 (d, J = 7.4 Hz, 3H), 0.66 (td, J = 6.9, 4.7 Hz, |
| | | | | 2H), 0.53-0.40 (m, |
| | | | | 2H). LCMS m/z |
| | | | | [M + H]⁺ 430.24 |
| 60 | 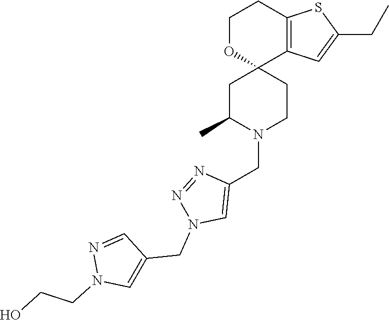 [SMILES: CCc1cc2c(s1)CCO[C@@]21CCN(Cc2cn(Cc3cnn(CCO)c3)nn2)[C@@H](C)C1] | 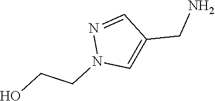 | Com- pound 15 | 1H NMR (400 MHz, DMSO-d6) δ 10.06 (s, 1H), 8.34 (s, 1H), 7.83 (s, 1H), 7.50 (s, 1H), 6.46 (s, 1H), 5.53 (s, 2H), 4.60 (d, J = 14.2 Hz, 1H), 4.48 (d, J = 14.9 Hz, 1H), 4.11 (t, J = 5.5 Hz, 2H), 3.81 (d, J = 7.2 Hz, 2H), 3.70 (t, J = 5.6 Hz, 2H), 3.63 (s, 2H), 3.30 (d, J = 11.8 Hz, 2H), 3.22-3.02 (m, 2H), 2.66 (d, J = 5.8 Hz, 2H), 2.01 (d, J = 14.7 Hz, 4H), 1.43 (d, J = |
| | | | | 6.4 Hz, 3H), 1.19 |
| | | | | (t, J = 7.5 Hz, 3H). |
| | | | | LCMS m/z [M + H]⁺ |
| | | | | 457.21 |
| 61 | 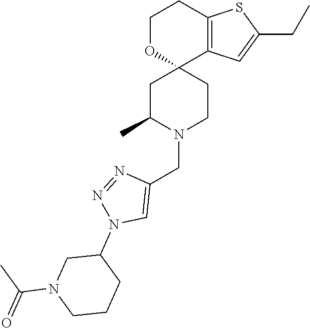 [SMILES: CCc1cc2c(s1)CCO[C@@]21CCN(Cc2cn(C3CCCN(C(C)=O)C3)nn2)[C@@H](C)C1] | 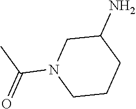 | Com- pound 15 | 1H NMR (400 MHz, DMSO-d6) δ 10.03 (s, 1H), 8.97 (s, 1H), 8.45 (d, J = 18.5 Hz, 1H), 6.46 (s, 1H), 4.79-4.36 (m, 4H), 4.04 (d, J = 16.7 Hz, 1H), 3.83 (t, J = 6.7 Hz, 2H), 3.76-3.57 (m, 2H), 3.33-3.07 (m, 5H), 2.68 (s, 2H), 2.18 (dd, J = 41.3, 10.7 Hz, 4H), 1.99 (d, J = 2.6 Hz, 3H), 1.79 (s, 1H), 1.70- 1.49 (m, 1H), 1.44 (d, J = 6.3 Hz, 3H), 1.20 (t, J = 7.6 Hz, 3H). LCMS m/z [M + H]⁺ 458.25 |
| 62 | 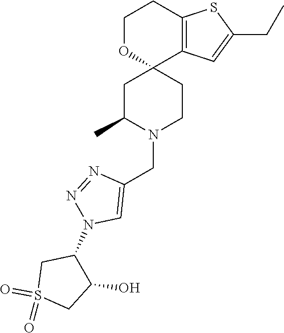 [SMILES: CCc1cc2c(s1)CCO[C@@]21CCN(Cc2cn([C@H]3CS(=O)(=O)C[C@H]3O)nn2)[C@@H](C)C1] | 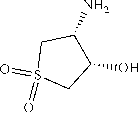 | Com- pound 15 | 1H NMR (400 MHz, DMSO-d6) δ 10.06 (s, 1H), 8.52 (s, 1H), 6.47 (s, 1H), 6.25 (d, J = 4.5 Hz, 1H), 5.72- 5.49 (m, 1H), 4.78 (s, 1H), 4.63 (d, J = 13.1 Hz, 1H), 4.55 (s, 1H), 4.05-3.86 (m, 2H), 3.82 (q, J = 6.3 Hz, 2H), 3.63 (dd, J = 13.7, 4.6 Hz, 1H), 3.41 (d, J = 13.2 Hz, 3H), 3.31 (s, 1H), 3.20 (s, 1H), 2.67 (d, J = 5.6 Hz, 2H), 2.17- 1.92 (m, 4H), 1.44 (d, J = 6.3 Hz, 3H), 1.20 (t, J = 7.5 Hz, |
| | | | | 3H). LCMS LCMS |
| | | | | m/z [M + H]⁺ 467.15 |
| 63 | 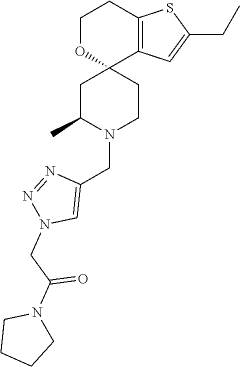 [SMILES: CCc1cc2c(s1)CCO[C@@]21CCN(Cc2cn(CC(=O)N3CCCC3)nn2)[C@@H](C)C1] | 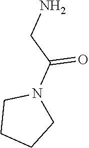 | Com- pound 15 | 1H NMR (400 MHz, DMSO-d6) δ 10.06 (s, 1H), 8.28 (s, 1H), 6.47 (s, 1H), 5.45 (s, 2H), 4.66 (d, J = 14.1 Hz, 1H), 4.56 (d, J = 13.8 Hz, 1H), 3.82 (q, J = 6.1 Hz, 2H), 3.56 (s, 4H), 3.33 (t, J = 6.9 Hz, 4H), 3.19 (s, 1H), 2.68 (d, J = 6.1 Hz, 2H), 2.04 (d, J = 9.6 Hz, 4H), 1.94 (p, J = 7.0 Hz, 2H), 1.81 (p, J = 6.8 Hz, 2H), 1.46 (d, J = 6.4 Hz, 3H), 1.20 (t, J = 7.5 Hz, 3H). LCMS LCMS m/z [M + H]⁺ 444.21 |
| 64 | 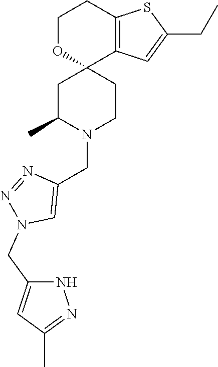 [SMILES: CCc1cc2c(s1)CCO[C@@]21CCN(Cc2cn(Cc3cc(C)n[nH]3)nn2)[C@@H](C)C1] | 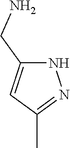 | Com- pound 15 | 1H NMR (400 MHz, DMSO-d6) δ 10.03 (s, 1H), 8.32 (s, 1H), 6.46 (s, 1H), 5.99 (s, 1H), 5.56 (s, 2H), 4.61 (d, J = 14.2 Hz, 1H), 4.49 (d, J = 15.4 Hz, 1H), 3.81 (q, J = 6.0 Hz, 3H), 3.31 (d, J = 12.0 Hz, 2H), 3.21- 3.06 (m, 2H), 2.67 (s, 2H), 2.20 (s, 3H), 2.03 (t, J = 9.5 Hz, 4H), 1.44 (d, J = 6.3 Hz, 3H), 1.25 (q, J = 7.3, 6.8 Hz, 3H). LCMS LCMS m/z [M + H]⁺ 427.24 |
| 65 | 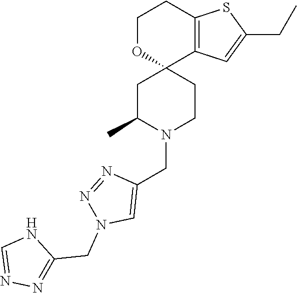 [SMILES: CCc1cc2c(s1)CCO[C@@]21CCN(Cc2cn(Cc3nnc[nH]3)nn2)[C@@H](C)C1] | 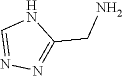 | Com- pound 15 | 1H NMR (400 MHz, DMSO-d6) δ 14.19 (s, 1H), 10.03 (s, 1H), 8.57 (s, 1H), 8.41 (s, 1H), 6.46 (s, 1H), 5.78 (s, 2H), 4.64 (d, J = 14.1 Hz, 1H), 4.52 (d, J = 14.7 Hz, 1H), 3.83 (tt, J = 12.1, 5.9 Hz, 2H), 3.62 (d, J = 5.5 Hz, 2H), 3.32 (d, J = 12.2 Hz, 2H), 3.24-3.04 (m, 2H), 2.69 (d, J = 15.0 Hz, 2H), 2.02 (d, J = 11.2 Hz, |
| | | | | 3H), 1.44 (d, J = |
| | | | | 6.3 Hz, 3H), 1.26 |
| | | | | (t, J = 6.2 Hz, 3H). |
| | | | | LCMS LCMS m/z |
| | | | | [M + H]⁺ 414.21 |
| 66 | 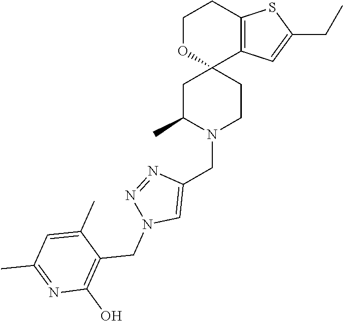 [SMILES: CCc1cc2c(s1)CCO[C@@]21CCN(Cc2cn(Cc3c(C)cc(C)nc3O)nn2)[C@@H](C)C1] | 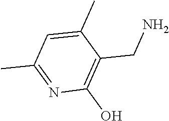 | Com- pound 15 | 1H NMR (400 MHz, DMSO-d6) δ 11.75 (s, 1H), 10.00 (s, 1H), 8.25 (s, 1H), 6.46 (s, 1H), 5.96 (s, 1H), 5.42 (s, 2H), 4.60 (d, J = 14.2 Hz, 1H), 4.45 (d, J = 14.9 Hz, 1H), 3.82 (dq, J = 12.5, 6.0 Hz, 2H), 3.70- 3.55 (m, 1H), 3.36- 3.06 (m, 4H), 2.67 (s, 2H), 2.25 (s, 3H), 2.14 (s, 3H), 2.01 (d, J = 12.0 Hz, 4H), 1.42 (d, J = 6.3 Hz, 3H), 1.19 (t, J = 7.4 Hz, 3H). |
| | | | | LCMS m/z [M + H]+ |
| | | | | 468.19 |
| 67 | 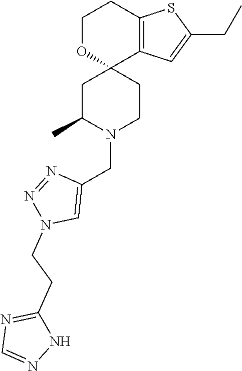 [SMILES: CCc1cc2c(s1)CCO[C@@]21CCN(Cc2cn(CCc3ncn[nH]3)nn2)[C@@H](C)C1] | 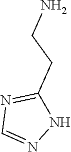 | Com- pound 15 | 1H NMR (400 MHz, DMSO-d6) δ 10.00 (s, 1H), 8.28 (s, 2H), 6.46 (s, 1H), 4.81 (t, J = 6.9 Hz, 2H), 4.59 (d, J = 14.2 Hz, 1H), 4.48 (d, J = 14.3 Hz, 1H), 3.68- 3.56 (m, 2H), 3.36- 3.17 (m, 5H), 3.10 (s, 2H), 2.68 (s, 2H), 2.16-1.94 (m, 4H), 1.42 (d, J = 6.3 Hz, 3H), 1.26 (t, J = 6.2 Hz, 3H). LCMS m/z [M + H]+ 428.22 |
| 68 | 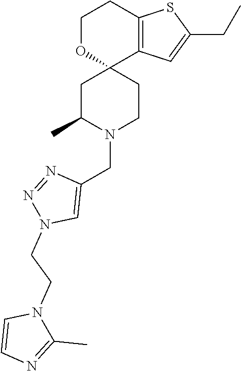 [SMILES: CCc1cc2c(s1)CCO[C@@]21CCN(Cc2cn(CCn3ccnc3C)nn2)[C@@H](C)C1] | 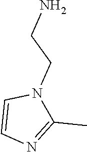 | Com- pound 15 | 1H NMR (400 MHz, DMSO-d6) δ 8.98 (s, 1H), 8.34 (s, 1H), 7.52 (d, J = 2.0 Hz, 1H), 7.39 (d, J = 2.1 Hz, 1H), 6.49 (s, 1H), 4.94 (t, J = 5.7 Hz, 2H), 4.67 (d, J = 5.8 Hz, 2H), 4.57 (s, 2H), 3.89-3.73 (m, 2H), 3.62 (s, 1H), 3.25 (d, J = 13.2 Hz, 2H), 3.16-3.04 (m, 2H), 2.68 (s, 2H), 2.35 (s, 3H), 2.02 (dd, J = 24.4, 11.1 Hz, 4H), 1.43 (d, J = 6.3 Hz, 3H), 1.20 (t, J = 7.7 Hz, 3H). LCMS m/z [M + H]+ 441.22 |
| 69 | 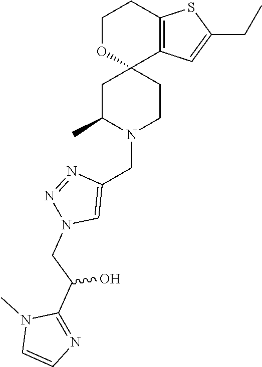 [SMILES: CCc1cc2c(s1)CCO[C@@]21CCN(Cc2cn(CC(O)c3nccn3C)nn2)[C@@H](C)C1] | 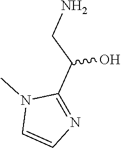 | Com- pound 15 | 1H NMR (400 MHz, DMSO-d6) δ 8.97 (s, 1H), 8.36 (d, J = 4.5 Hz, 1H), 7.63 (s, 1H), 7.49 (s, 1H), 6.48 (s, 1H), 5.54 (s, 1H), 4.92 (d, J = 5.3 Hz, 2H), 4.68-4.50 (m, 2H), 3.82 (s, 5H), 3.62 (s, 2H), 3.28 (s, 2H), 3.14 (dd, J = 7.5, 4.2 Hz, 2H), 2.68 (s, 2H), 2.07 (d, J = 27.6 Hz, 4H), 1.44 (d, J = 6.3 Hz, 3H), 1.25 (q, J = 7.1, 6.7 Hz, 3H). LCMS m/z [M + H]+ 457.21 |
| 70 | 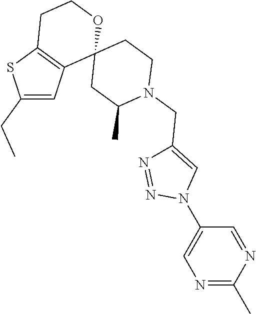 [SMILES: CCc1cc2c(s1)CCO[C@@]21CCN(Cc2cn(-c3cnc(C)nc3)nn2)[C@@H](C)C1] | 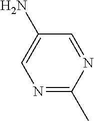 | Com- pound 15 | 1H NMR (400 MHz, Chloroform- d) δ 9.09 (s, 2H), 7.98 (s, 1H), 6.48 (d, J = 1.1 Hz, 1H), 4.17 (d, J = 14.8 Hz, 1H), 4.01- 3.78 (m, 3H), 2.86 (s, 3H), 2.84-2.60 (m, 7H), 1.99-1.84 (m, 3H), 1.73 (dd, J = 13.9, 11.4 Hz, 1H), 1.33-1.23 (m, 6H). LCMS m/z [M + H]+ 425.18 |
| 71 | 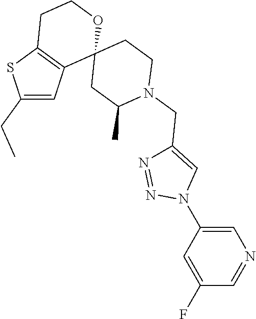 [SMILES: CCc1cc2c(s1)CCO[C@@]21CCN(Cc2cn(-c3cncc(F)c3)nn2)[C@@H](C)C1] | 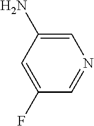 | Com- pound 16 | 1H NMR (400 MHz, Chloroform- d) δ 8.78 (dt, J = 2.1, 0.7 Hz, 1H), 8.51 (dd, J = 2.6, 0.5 Hz, 1H), 7.98- 7.89 (m, 2H), 6.40 (d, J = 1.1 Hz, 1H), 4.12-4.04 (m, 1H), 3.88 (d, J = 14.7 Hz, 1H), 3.85- 3.72 (m, 2H), 2.75- 2.52 (m, 7H), 1.90- 1.73 (m, 3H), 1.65 (dd, J = 13.9, 11.4 Hz, 1H), 1.24- 1.16 (m, 6H). LCMS m/z [M + H]+ 428.1 |
| 72 | 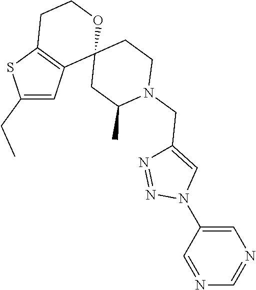 [SMILES: CCc1cc2c(s1)CCO[C@@]21CCN(Cc2cn(-c3cncnc3)nn2)[C@@H](C)C1] | 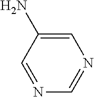 | Com- pound 16 | 1H NMR (400 MHz, Chloroform- d) δ 9.24 (s, 1H), 9.15 (s, 2H), 7.94 (s, 1H), 6.40 (d, J = 1.1 Hz, 1H), 4.10 (d, J = 14.8 Hz, 1H), 3.89 (d, J = 14.7 Hz, 1H), 3.87- 3.73 (m, 2H), 2.77- 2.53 (m, 7H), 1.90- 1.71 (m, 3H), 1.68 (s, 1H), 1.24-1.15 (m, 6H). LCMS m/z [M + H]+ 411.13 |
| 73 | 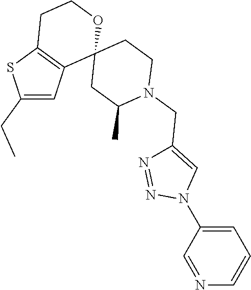 [SMILES: CCc1cc2c(s1)CCO[C@@]21CCN(Cc2cn(-c3cccnc3)nn2)[C@@H](C)C1] | 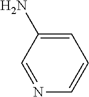 | Com- pound 16 | 1H NMR (400 MHz, Chloroform- d) δ 8.95 (dd, J = 2.7, 0.7 Hz, 1H), 8.63 (dd, J = 4.8, 1.5 Hz, 1H), 8.10 (ddd, J = 8.3, 2.6, 1.5 Hz, 1H), 7.92 (s, 1H), 7.43 (ddd, J = 8.3, 4.8, 0.8 Hz, 1H), 6.40 (t, J = 1.1 Hz, 1H), 4.08 (d, J = 14.7 Hz, 1H), 3.88 (d, J = 14.7 Hz, 1H), 3.85- 3.72 (m, 2H), 2.78- 2.52 (m, 7H), 1.90- 1.74 (m, 3H), 1.65 |
| | | | | (dd, J = 14.0, 11.3 |
| | | | | Hz, 1H), 1.24- |
| | | | | 1.15 (m, 6H). |
| | | | | LCMS m/z [M + H]+ |
| | | | | 410.19 |
| 74 | 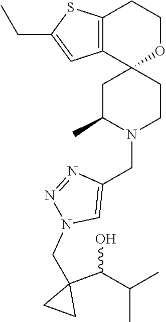 [SMILES: CCc1cc2c(s1)CCO[C@@]21CCN(Cc2cn(CC3(C(O)C(C)C)CC3)nn2)[C@@H](C)C1] | 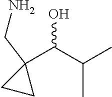 | Com- pound 15 | LCMS m/z [M + H]+ 459.3 |
| 75 | 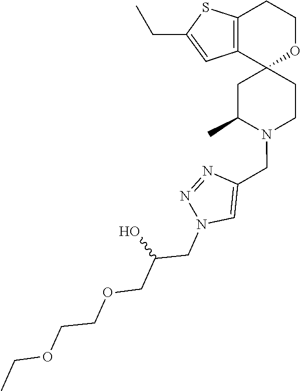 | 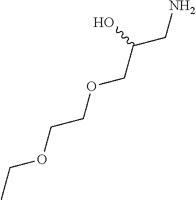 | Com- pound 15 | LCMS m/z [M + H]+ 479.3 |
| 76 | 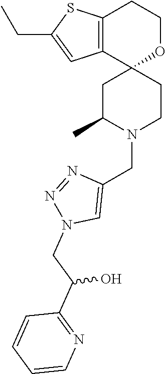 | 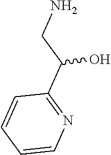 | Com- pound 15 | LCMS m/z [M + H]+ 454.21 |
| 77 | 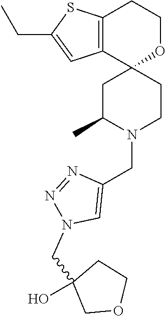 | 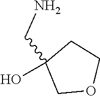 | Com- pound 15 | LCMS m/z [M + H]+ 433.24 |
| 78 | 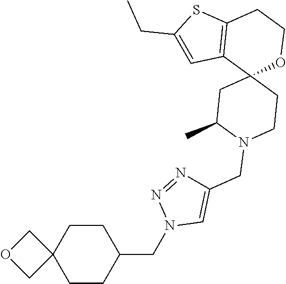 | 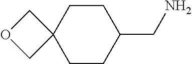 [SMILES: NCC1CCC2(CC1)COC2] | Com- pound 15 | LCMS m/z [M + H]+ 471.28 |
| 79 | 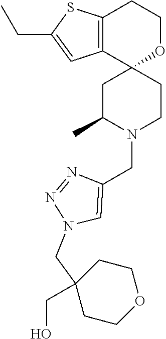 | 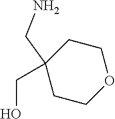 | Com- pound 15 | LCMS m/z [M + H]+ 461.28 |
| 80 | 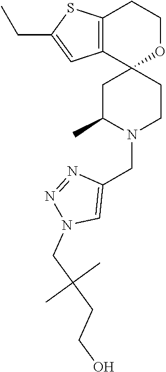 | 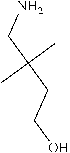 | Com- pound 15 | LCMS m/z [M + H]+ 433.3 |
| 81 | 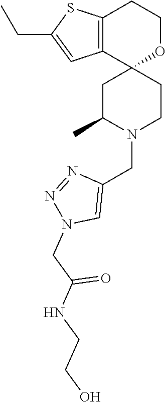 | 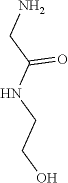 | Com- pound 15 | LCMS m/z [M + H]+ 434.21 |
| 82 | 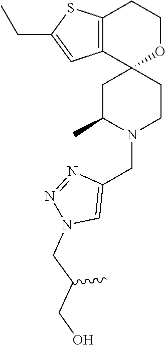 | 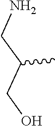 | Com- pound 15 | LCMS m/z [M + H]+ 405.22 |
| 83 | 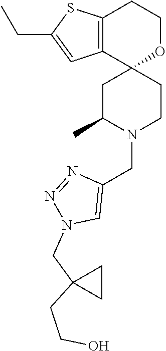 | 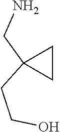 | Com- pound 15 | LCMS m/z [M + H]+ 431.28 |
| 84 | 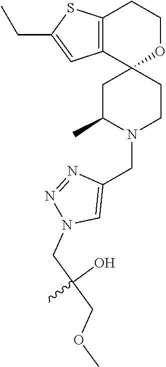 | 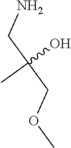 | Com- pound 15 | LCMS m/z [M + H]+ 435.29 |
| 85 | 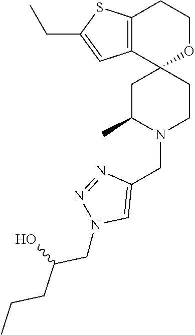 [SMILES: CCCC(O)Cn1cc(CN2CC[C@]3(C[C@@H]2C)OCCc2sc(CC)cc23)nn1] | 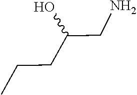 | Com- pound 15 | LCMS m/z [M + H]+ 419.26 |
| 86 | 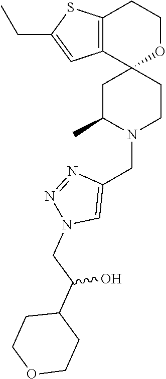 | 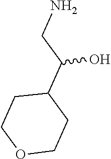 | Com- pound 15 | LCMS m/z [M + H]+ 461.28 |
| 87 | 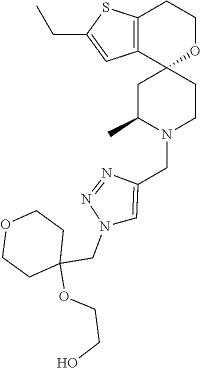 | 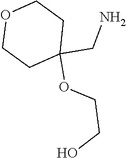 [SMILES: NCC1(OCCO)CCOCC1] | Com- pound 15 | LCMS m/z [M + H]+ 491.28 |
| 88 | 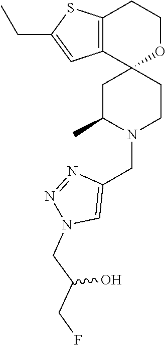 | 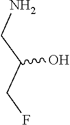 | Com- pound 15 | LCMS m/z [M + H]+ 409.23 |
| 89 | 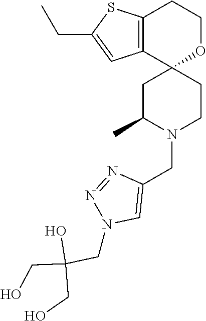 [SMILES: CCc1cc2c(s1)CCO[C@@]21CCN(Cc2cn(CC(O)(CO)CO)nn2)[C@@H](C)C1] | 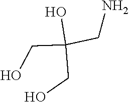 | Com- pound 15 | LCMS m/z [M + H]+ 437.24 |
| 90 | 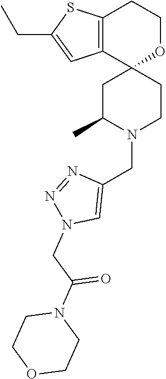 | 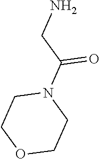 | Com- pound 15 | LCMS m/z [M + H]+ 460.24 |
| 91 | 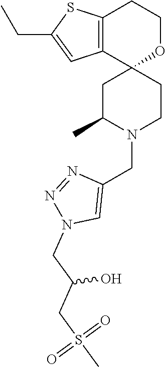 | 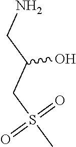 | Com- pound 15 | LCMS m/z [M + H]+ 469.2 |
| 92 | 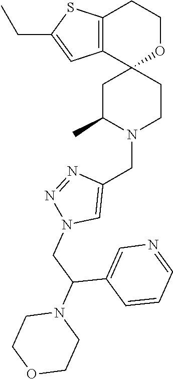 | 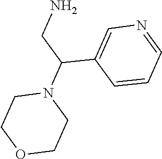 [SMILES: NCC(c1cccnc1)N1CCOCC1] | Com- pound 15 | LCMS m/z [M + H]+ 523.3 |
| 93 | 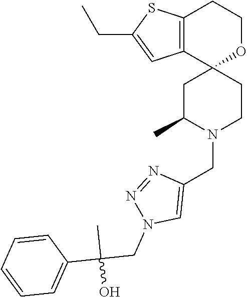 | 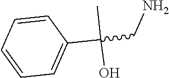 | Com- pound 15 | LCMS m/z [M + H]+ 467.28 |
| 94 | 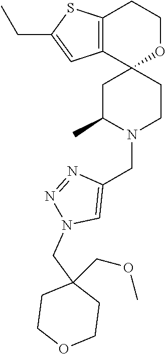 | 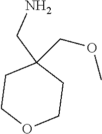 | Com- pound 15 | LCMS m/z [M + H]+ 475.32 |
| 95 | 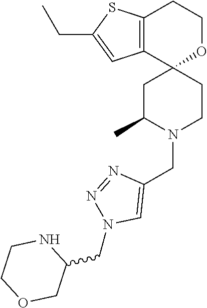 [SMILES: CCc1cc2c(s1)CCO[C@@]21CCN(Cc2cn(CC3COCCN3)nn2)[C@@H](C)C1] | 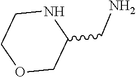 | Com- pound 15 | LCMS m/z [M + H]+ 432.23 |
| 96 | 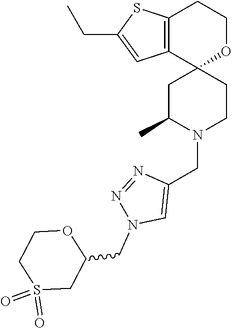 [SMILES: CCc1cc2c(s1)CCO[C@@]21CCN(Cc2cn(CC3CS(=O)(=O)CCO3)nn2)[C@@H](C)C1] | 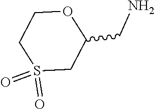 | Com- pound 15 | LCMS m/z [M + H]+ 481.19 |
| 97 | 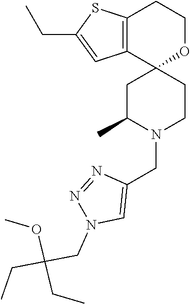 [SMILES: CCc1cc2c(s1)CCO[C@@]21CCN(Cc2cn(CC(CC)(CC)OC)nn2)[C@@H](C)C1] | 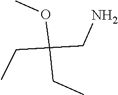 | Com- pound 15 | LCMS m/z [M + H]+ 447.28 |
| 98 | 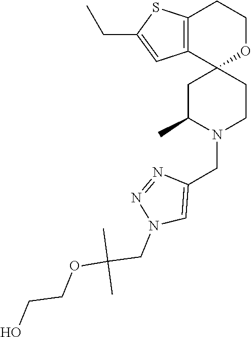 | 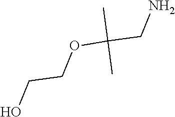 | Com- pound 15 | 1H NMR (400 MHz, Chloroform- d) δ 7.86 (s, 1H), 6.47 (t, J = 1.1 Hz, 1H), 4.38 (s, 2H), 4.12 (d, J = 14.4 Hz, 1H), 3.94- 3.78 (m, 3H), 3.78- 3.71 (m, 2H), 3.53 (ddt, J = 4.2, 3.3, 2.1 Hz, 2H), 2.80- 2.60 (m, 8H), 1.94 (td, J = 13.3, 12.5, 4.7 Hz, 1H), 1.88- 1.71 (m, 3H), 1.25 (td, J = 6.8, 6.1, 4.8 Hz, 6H), 1.20 (s, 3H), 1.17 (s, 3H). LCMS m/z [M + H]+ 449.23 |
| 99 | 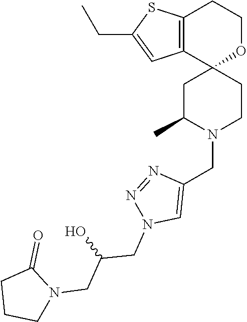 | 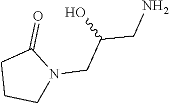 [SMILES: NCC(O)CN1CCCC1=O] | Com- pound 15 | LCMS m/z [M + H]+ 474.28 |
| 100 | 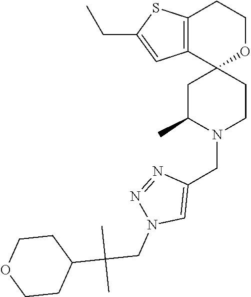 | 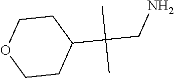 | Com- pound 15 | LCMS m/z [M + H]+ 473.21 |
| 101 | 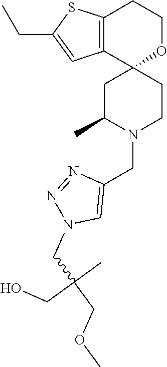 | 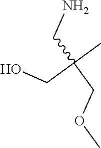 | Com- pound 15 | 1H NMR (400 MHz, Chloroform- d) δ 7.58 (d, J = 2.7 Hz, 1H), 6.47 (d, J = 1.1 Hz, 1H), 4.53- 4.39 (m, 2H), 4.07 (dd, J = 14.5, 2.4 Hz, 1H), 3.94- 3.79 (m, 3H), 3.52 (d, J = 11.6 Hz, 1H), 3.49 (s, 1H), 3.39 (s, 3H), 3.35- 3.28 (m, 2H), 3.22 (d, J = 9.4 Hz, 1H), 2.80-2.57 (m, 6H), 1.98-1.78 (m, 3H), 1.72 (ddd, J = 13.9, 11.4, 2.5 Hz, 1H), 1.33-1.21 (m, 6H), 0.88 (d, J = 0.6 Hz, 3H). LCMS m/z [M + H]+ 449.64 |
| 102 | 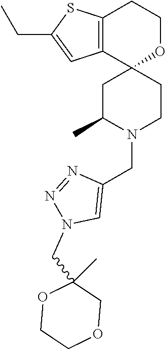 | 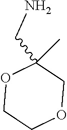 | Com- pound 15 | 1H NMR (400 MHz, Chloroform- d) δ 7.58 (d, J = 2.7 Hz, 1H), 6.47 (d, J = 1.1 Hz, 1H), 4.53- 4.39 (m, 2H), 4.07 (dd, J = 14.5, 2.4 Hz, 1H), 3.94- 3.79 (m, 3H), 3.52 (d, J = 11.6 Hz, 1H), 3.49 (s, 1H), 3.39 (s, 3H), 3.35- 3.28 (m, 2H), 3.22 (d, J = 9.4 Hz, 1H), 2.80-2.57 (m, 8H), 1.98-1.78 (m, 3H), 1.72 (ddd, J = 13.9, 11.4, 2.5 Hz, 1H), 1.33-1.21 (m, 6H), 0.88 (d, J = 0.6 Hz, 3H). LCMS m/z [M + H]+ 447.61 |
| 103 | 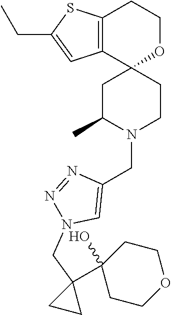 | 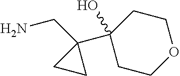 [SMILES: NCC1(C2(O)CCOCC2)CC1] | Com- pound 15 | LCMS m/z [M + H]+ 487.24 |
| 104 | 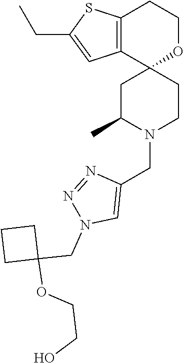 | 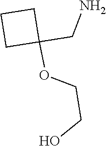 | Com- pound 15 | LCMS m/z [M + H]+ 461.28 |
| 105 | 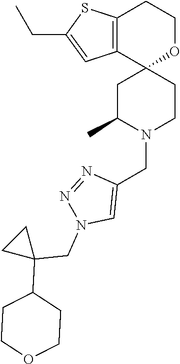 | 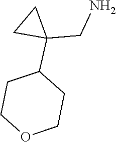 [SMILES: NCC1(C2CCOCC2)CC1] | Com- pound 15 | LCMS m/z [M + H]+ 471.28 |
| 106 | 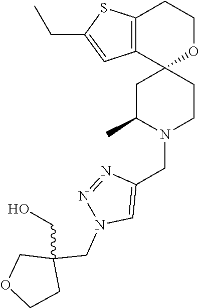 | 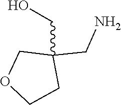 | Com- pound 15 | LCMS m/z [M + H]+ 447.24 |
| 107 | 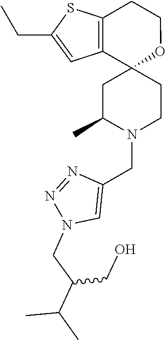 | 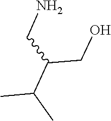 | Com- pound 15 | LCMS m/z [M + H]+ 433.3 |
| 108 | 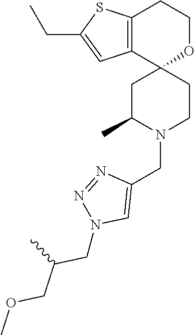 [SMILES: CCc1cc2c(s1)CCO[C@@]21CCN(Cc2cn(CC(C)COC)nn2)[C@@H](C)C1] | 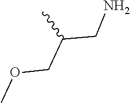 | Com- pound 15 | LCMS m/z [M + H]+ 419.29 |
| 109 | 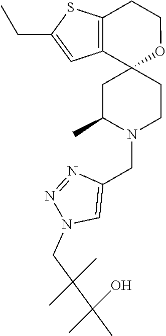 | 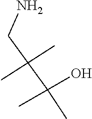 | Com- pound 15 | LCMS m/z [M + H]+ 447.28 |
| 110 | 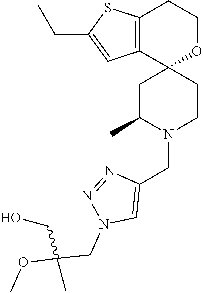 | 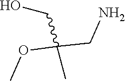 | Com- pound 15 | LCMS m/z [M + H]+ 435.29 |
| 111 | 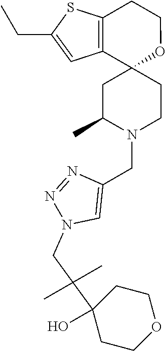 | 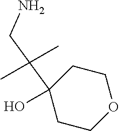 | Com- pound 15 | LCMS m/z [M + H]+ 489.3 |
| 112 | 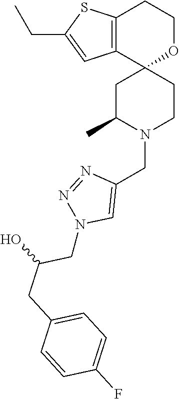 [SMILES: CCc1cc2c(s1)CCO[C@@]21CCN(Cc2cn(CC(O)Cc3ccc(F)cc3)nn2)[C@@H](C)C1] | 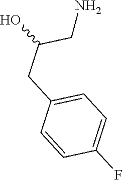 [SMILES: NCC(O)Cc1ccc(F)cc1] | Com- pound 15 | LCMS m/z [M + H]+ 485.26 |
| 113 | 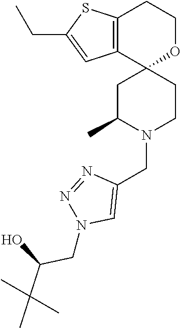 [SMILES: CCc1cc2c(s1)CCO[C@@]21CCN(Cc2cn(C[C@H](O)C(C)(C)C)nn2)[C@@H](C)C1] | 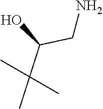 | Com- pound 15 | LCMS m/z [M + H]+ 433.3 |
| 114 | 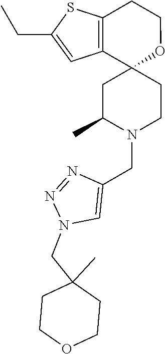 | 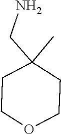 | Com- pound 15 | LCMS m/z [M + H]+ 445.29 |
| 115 | 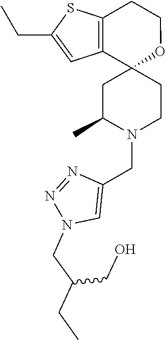 | 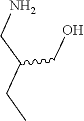 | Com- pound 15 | LCMS m/z [M + H]+ 419.26 |
| 116 | 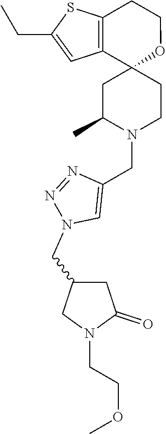 | 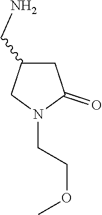 | Com- pound 15 | LCMS m/z [M + H]+ 488.29 |
| 117 | 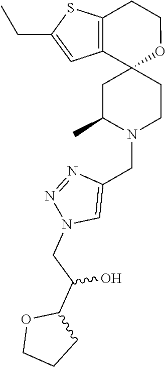 | 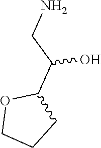 | Com- pound 15 | LCMS m/z [M + H]+ 447.28 |
| 118 | 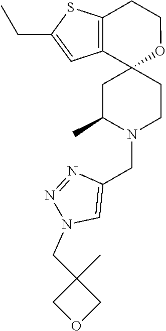 | 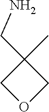 | Com- pound 15 | LCMS m/z [M + H]+ 417.24 |
| 119 | 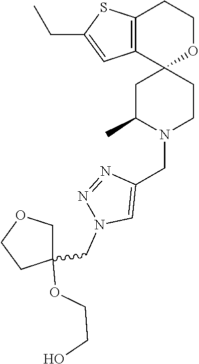 | 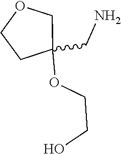 [SMILES: NCC1(OCCO)CCOC1] | Com- pound 15 | LCMS m/z [M + H]+ 477.28 |
| 120 | 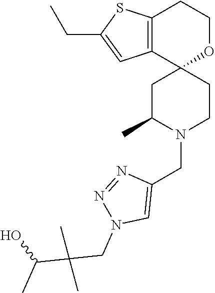 | 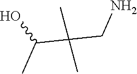 | Com- pound 15 | LCMS m/z [M + H]+ 433.27 |
| 121 | 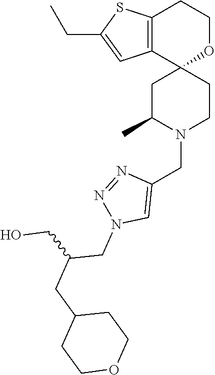 | 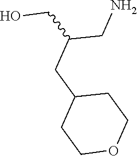 [SMILES: NCC(CO)CC1CCOCC1] | Com- pound 15 | LCMS m/z [M + H]+ 489.3 |
| 122 | 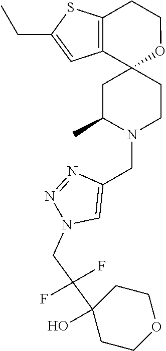 | 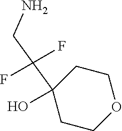 [SMILES: NCC(F)(F)C1(O)CCOCC1] | Com- pound 15 | LCMS m/z [M + H]+ 497.21 |
| 123 | 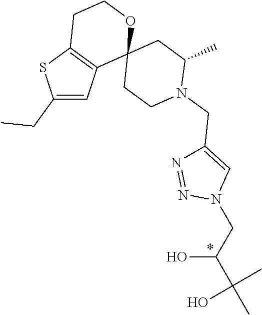 | 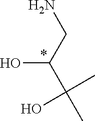 | Com- pound 15 | 1H NMR (400 MHz, Chloroform- d) δ 7.65 (s, 1H), 6.48 (d, J = 1.1 Hz, 1H), 4.64 (dd, J = 13.9, 2.3 Hz, 1H), 4.31 (dd, J = 13.9, 9.4 Hz, 1H), 4.08 (d, J = 14.5 Hz, 1H), 3.95-3.78 (m, 4H), 3.49 (d, J = 17.0 Hz, 1H), 2.86- 2.57 (m, 7H), 2.20 (s, 1H), 1.97-1.81 (m, 3H), 1.75 (d, J = 12.1 Hz, 1H), 1.41-1.19 (m, 12H). [2] LCMS m/z [M + H]+ 435.55 |
| 124 | 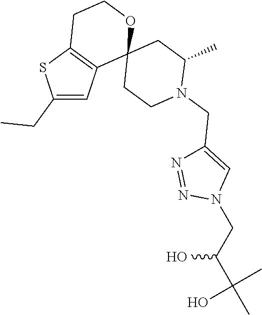 | 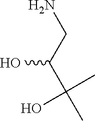 | Com- pound 15 | 1H NMR (400 MHz, Chloroform- d) δ 7.64 (s, 1H), 6.48 (s, 1H), 4.64 (dt, J = 13.9, 2.5 Hz, 1H), 4.32 (ddd, J = 13.9, 9.3, 2.2 Hz, 1H), 4.08 (d, J = 15.2 Hz, 1H), 3.98-3.73 (m, 4H), 3.34 (s, 1H), 2.87- 2.53 (m, 6H), 2.00- 1.80 (m, 3H), 1.80- 1.49 (m, 5H), 1.41- 1.32 (m, 5H), 1.28 (dd, J = 9.0, 6.0 Hz, 5H). [3] LCMS m/z [M + H]+ 435.6 |
| 125 | 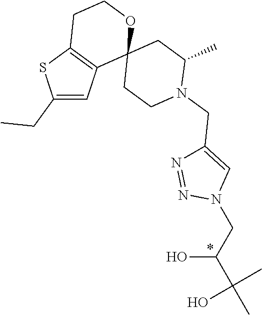 | 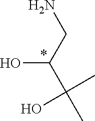 | Com- pound 15 | 1H NMR (400 MHz, Chloroform- d) δ 7.64 (s, 1H), 6.48 (d, J = 1.1 Hz, 1H), 4.65 (dd, J = 13.9, 2.3 Hz, 1H), 4.31 (dd, J = 13.9, 9.4 Hz, 1H), 4.06 (d, J = 14.5 Hz, 1H), 3.91-3.81 (m, 3H), 3.48 (d, J = 11.3 Hz, 1H), 2.88- 2.49 (m, 7H), 2.21 (d, J = 8.0 Hz, 1H), 1.98-1.81 (m, 3H), 1.80-1.72 (m, 2H), 1.41-1.12 (m, 12H). [2] LCMS m/z [M + H]+ 435.6 |
| 126 | 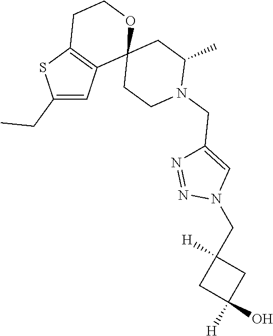 [SMILES: CCc1cc2c(s1)CCO[C@@]21CCN(Cc2cn(C[C@H]3C[C@@H](O)C3)nn2)[C@@H](C)C1] | 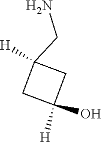 | Com- pound 15 | 1H NMR (400 MHz, DMSO-d6) δ 7.93 (s, 1H), 6.58 (s, 1H), 5.05 (d, J = 6.3 Hz, 1H), 4.34 (d, J = 6.5 Hz, 2H), 3.92 (q, J = 6.9 Hz, 1H), 3.84 (d, J = 14.4 Hz, 1H), 3.80- 3.66 (m, 3H), 2.70 (q, J = 7.5 Hz, 2H), 2.62 (d, J = 6.1 Hz, 2H), 2.57 (s, 1H), 2.42 (t, J = 11.3 Hz, 2H), 2.18 (tt, J = 15.1, 7.4 Hz, 3H), 1.76 (dt, J = 12.5, 6.7 Hz, 1H), 1.68 (d, J = 12.9 Hz, 2H), 1.63-1.48 (m, 3H), 1.19 (t, J = 7.5 |
| | | | | Hz, 3H), 1.12 (d, |
| | | | | J = 6.1 Hz, 3H). |
| | | | | LCMS m/z [M + H]+ |
| | | | | 417.6 |
| 127 | 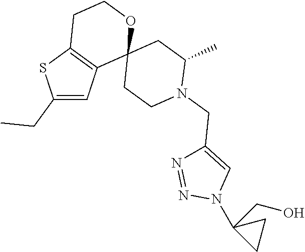 [SMILES: CCc1cc2c(s1)CCO[C@@]21CCN(Cc2cn(C3(CO)CC3)nn2)[C@@H](C)C1] | 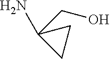 | Com- pound 15 | 1H NMR (400 MHz, DMSO-d6) δ 7.98 (s, 1H), 6.59 (s, 1H), 5.12 (t, J = 5.7 Hz, 1H), 3.87 (d, J = 14.3 Hz, 1H), 3.77 (h, J = 6.0 Hz, 2H), 3.68 (d, J = 5.6 Hz, 2H), 3.59 (d, J = 14.3 Hz, 1H), 2.70 (q, J = 7.6 Hz, 2H), 2.61 (d, J = 5.6 Hz, 3H), 2.41 (dd, J = 22.7, 11.0 Hz, 2H), 1.77 (td, J = 12.8, 12.1, |
| | | | | 4.2 Hz, 1H), 1.69 |
| | | | | (d, J = 9.1 Hz, 2H), |
| | | | | 1.55 (t, J = 12.5 Hz, |
| | | | | 1H), 1.23 (t, J = 3.5 |
| | | | | Hz, 2H), 1.19 (dd, |
| | | | | J = 8.1, 6.9 Hz, 3H), |
| | | | | 1.12 (d, J = 5.4 Hz, |
| | | | | 5H). LCMS m/z |
| | | | | [M + H]+ 403.56 |
| 128 | 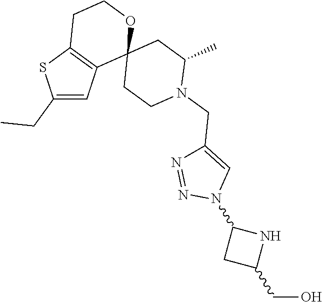 [SMILES: CCc1cc2c(s1)CCO[C@@]21CCN(Cc2cn(C3CC(CO)N3)nn2)[C@@H](C)C1] | 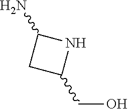 | Com- pound 15 | 1H NMR (400 MHz, DMSO-d6) δ 9.80 (s, 1H), 8.97 (s, 1H), 8.46 (d, J = 20.5 Hz, 1H), 6.45 (s, 1H), 5.34-5.01 (m, 1H), 4.70 (s, 1H), 4.63 (d, J = 14.2 Hz, 1H), 4.47 (d, J = 14.3 Hz, 1H), 3.93-3.68 (m, 2H), 3.59-3.42 (m, 3H), 3.20 (d, J = 16.0 Hz, 1H), 2.83- 2.64 (m, 4H), 2.39 (d, J = 9.3 Hz, 1H), 2.30 (d, J = 6.4 Hz, 2H), 2.00 (t, J = 14.0 Hz, 4H), 1.44 |
| | | | | (d, J = 6.3 Hz, 3H), |
| | | | | 1.36 (d, J = 11.6 |
| | | | | Hz, 1H), 1.21 (q, |
| | | | | J = 7.5 Hz, 4H). |
| | | | | LCMS m/z [M + H]+ |
| | | | | 417.27 |
| 129 | 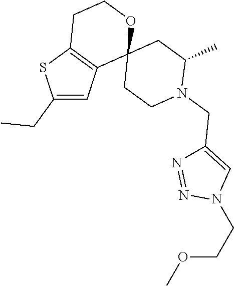 [SMILES: CCc1cc2c(s1)CCO[C@@]21CCN(Cc2cn(CCOC)nn2)[C@@H](C)C1] | 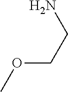 | Com- pound 15 | 1H NMR (400 MHz, DMSO-d6) δ 7.96 (s, 1H), 6.58 (s, 1H), 4.52 (t, J = 5.3 Hz, 2H), 3.88 (s, 1H), 3.75 (dt, J = 14.7, 5.4 Hz, 5H), 3.23 (d, J = 1.4 Hz, 3H), 2.70 (q, J = 7.6 Hz, 2H), 2.61 (d, J = 6.0 Hz, 3H), 2.47 (d, J = 18.3 Hz, 2H), 1.78 (s, 1H), 1.73-1.62 (m, 2H), 1.56 (t, J = 12.4 Hz, 1H), 1.22- 1.16 (m, 3H), 1.14 (d, J = 6.2 Hz, 3H). |
| | | | | LCMS m/z [M + H]+ |
| | | | | 391.54 |
| 130 | 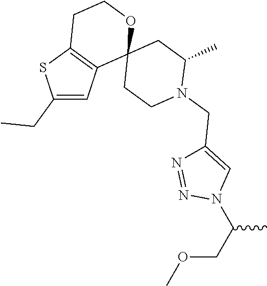 [SMILES: CCc1cc2c(s1)CCO[C@@]21CCN(Cc2cn(C(C)COC)nn2)[C@@H](C)C1] | 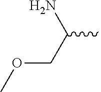 | Com- pound 15 | 1H NMR (400 MHz, DMSO-d6) δ 9.98 (s, 1H), 8.40 (d, J = 2.3 Hz, 1H), 6.46 (s, 1H), 4.97 (q, J = 6.6 Hz, 1H), 4.63 (d, J = 14.3 Hz, 1H), 4.50 (d, J = 15.7 Hz, 1H), 3.82 (h, J = 6.0 Hz, 2H), 3.74-3.63 (m, 2H), 3.32 (d, J = 11.9 Hz, 2H), 3.22 (s, 3H), 3.15 (s, 1H), 2.74 (q, J = 7.5 Hz, 2H), 2.68 (d, J = 5.9 Hz, 2H), 2.04 (dd, J = 12.6, |
| | | | | 7.8 Hz, 4H), 1.50 |
| | | | | (d, J = 6.9 Hz, 3H), |
| | | | | 1.45 (d, J = 6.3 Hz, |
| | | | | 3H), 1.20 (t, J = 7.5 |
| | | | | Hz, 3H). LCMS |
| | | | | m/z [M + H]+ |
| | | | | 405.25 |
| 131 | 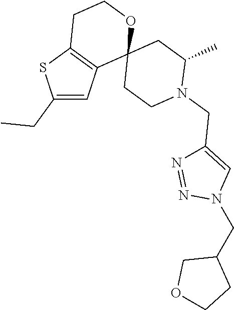 [SMILES: CCc1cc2c(s1)CCO[C@@]21CCN(Cc2cn(CC3CCOC3)nn2)[C@@H](C)C1] | 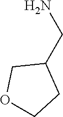 | Com- pound 15 | 1H NMR (400 MHz, DMSO-d6) δ 9.83 (s, 1H), 8.42 (s, 1H), 6.45 (s, 1H), 4.64 (d, J = 14.3 Hz, 1H), 4.52 (d, J = 14.4 Hz, 1H), 4.46 (d, J = 7.5 Hz, 2H), 3.87- 3.70 (m, 3H), 3.65 (p, J = 7.7 Hz, 2H), 3.49 (d, J = 6.7 Hz, 1H), 3.14 (s, 1H), 2.75 (p, J = 7.5 Hz, 4H), 2.67 (s, 2H), 2.10-1.87 (m, 5H), 1.62 (dd, J = 12.8, 6.5 Hz, 1H), 1.44 (d, J = 6.4 Hz, 3H), |
| | | | | 1.24 (s, 1H), 1.20 |
| | | | | (t, J = 7.5 Hz, 3H). |
| | | | | LCMS m/z [M + H]+ |
| | | | | 417.24 |
| 132 | 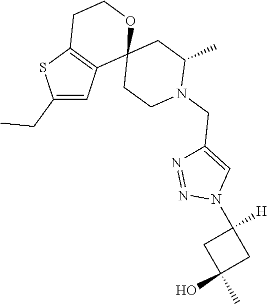 [SMILES: CCc1cc2c(s1)CCO[C@@]21CCN(Cc2cn([C@H]3C[C@@](C)(O)C3)nn2)[C@@H](C)C1] | 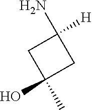 | Com- pound 15 | 1H NMR (400 MHz, DMSO-d6) δ 9.86-9.76 (m, 1H), 8.42 (s, 1H), 6.45 (s, 1H), 5.39 (s, 1H), 5.01-4.71 (m, 1H), 4.64 (d, J = 14.2 Hz, 1H), 4.48 (d, J = 14.2 Hz, 1H), 3.94-3.65 (m, 2H), 3.29-3.12 (m, 2H), 2.85-2.65 (m, 4H), 2.65-2.57 (m, 3H), 2.02 (d, J = 12.4 Hz, 4H), 1.44 (d, J = 6.3 Hz, 3H), 1.35 (s, 4H), 1.24 (s, 1H), 1.20 (t, J = 7.5 Hz, 3H). |
| | | | | LCMS m/z [M + H]+ |
| | | | | 417.27 |
| 133 | 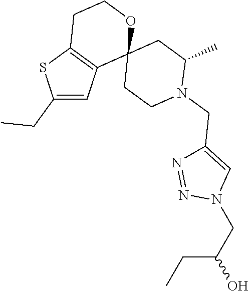 | 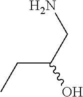 | Com- pound 15 | 1H NMR (400 MHz, DMSO-d6) δ 9.87 (s, 1H), 8.29 (d, J = 2.1 Hz, 1H), 6.46 (s, 1H), 5.11 (s, 1H), 4.63 (d, J = 14.3 Hz, 1H), 4.57- 4.39 (m, 2H), 4.31 (dd, J = 14.0, 7.3 Hz, 1H), 3.82 (td, J = 17.0, 14.4, 8.1 Hz, 3H), 3.29 (d, J = 1.8 Hz, 1H), 3.17 (d, J = 11.1 Hz, 1H), 2.88-2.58 (m, 4H), 2.02 (d, J = 11.4 Hz, 4H), 1.45 (d, J = 6.3 Hz, 3H), |
| | | | | 1.34 (tdd, J = 21.3, |
| | | | | 13.1, 7.4 Hz, 3H), |
| | | | | 1.21 (q, J = 7.9, 7.5 |
| | | | | Hz, 3H), 0.92 (t, |
| | | | | J = 7.4 Hz, 3H). |
| | | | | LCMS m/z [M + H]+ |
| | | | | 405.25 |
| 134 | 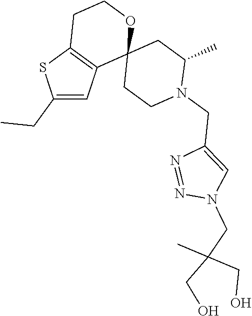 | 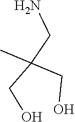 | Com- pound 15 | 1H NMR (400 MHz, DMSO-d6) δ 7.85 (s, 1H), 6.59 (s, 1H), 4.83-4.62 (m, 2H), 4.26 (s, 2H), 3.85 (d, J = 14.5 Hz, 1H), 3.80- 3.61 (m, 3H), 3.21 (t, J = 5.0 Hz, 4H), 2.70 (q, J = 7.5 Hz, 2H), 2.61 (s, 2H), 2.57 (s, 1H), 2.43 (t, J = 11.5 Hz, 2H), 1.77 (t, J = 12.5 Hz, 1H), 1.68 (d, J = 13.6 Hz, 2H), 1.54 (t, J = 12.3 Hz, 1H), 1.19 (td, J = 7.4, 1.8 Hz, 3H), 1.12 (d, J = |
| | | | | 6.1 Hz, 3H), 0.66 |
| | | | | (d, J = 1.8 Hz, 3H). |
| | | | | LCMS m/z [M + H]+ |
| | | | | 435.55 |
| 135 | 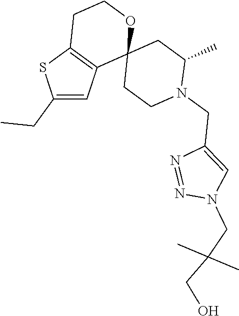 | 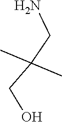 | Com- pound 15 | 1H NMR (400 MHz, DMSO-d6) δ 10.00 (s, 1H), 8.29 (s, 1H), 6.46 (s, 1H), 4.91 (d, J = 37.5 Hz, 1H), 4.75- 4.48 (m, 2H), 4.30 (s, 2H), 3.80 (h, J = 6.1 Hz, 2H), 3.29 (s, 3H), 3.14 (s, 3H), 2.82-2.63 (m, 3H), 2.02 (q, J = 14.8, 12.5 Hz, 3H), 1.46 (t, J = 7.3 Hz, 3H), 1.37-1.15 (m, 3H), 0.81 (s, 6H). LCMS m/z [M + H]+ 419.29 |
| 136 | 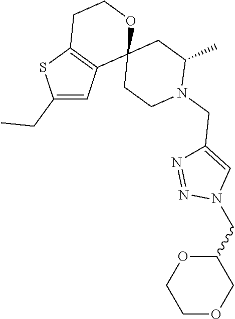 [SMILES: CCc1cc2c(s1)CCO[C@@]21CCN(Cc2cn(CC3COCCO3)nn2)[C@@H](C)C1] | 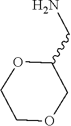 | Com- pound 15 | 1H NMR (400 MHz, DMSO-d6) δ 7.94 (s, 1H), 6.59 (s, 1H), 4.43 (d, J = 4.2 Hz, 2H), 3.96- 3.82 (m, 2H), 3.75 (d, J = 11.1 Hz, 5H), 3.64 (d, J = 11.5 Hz, 1H), 3.55 (t, J = 11.3 Hz, 1H), 3.44 (d, J = 11.3 Hz, 2H), 3.20 (t, J = 10.8 Hz, 1H), 2.76-2.65 (m, 2H), 2.62 (s, 2H), 2.43 (s, 2H), 1.76 (d, J = 12.9 Hz, 1H), 1.69 (d, J = 13.0 Hz, 2H), 1.55 (t, J = 12.4 Hz, |
| | | | | 1H), 1.19 (dt, J = |
| | | | | 9.9, 5.1 Hz, 3H), |
| | | | | 1.13 (d, J = 6.1 Hz, |
| | | | | 3H). LCMS m/z |
| | | | | [M + H]+ 433.53 |
| 137 | 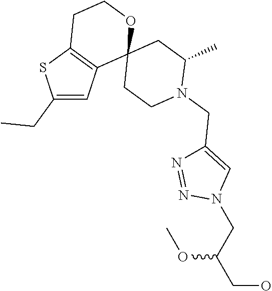 [SMILES: CCc1cc2c(s1)CCO[C@@]21CCN(Cc2cn(CC(CO)OC)nn2)[C@@H](C)C1] | 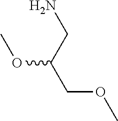 | Com- pound 15 | 1H NMR (400 MHz, DMSO-d6) δ 10.04 (s, 1H), 8.32 (d, J = 1.7 Hz, 1H), 6.47 (s, 1H), 4.69- 4.44 (m, 4H), 3.78 (dp, J = 12.7, 5.1, 4.3 Hz, 3H), 3.38- 3.36 (m, 1H), 3.35- 3.30 (m, 1H), 3.29 (s, 4H), 3.23 (d, J = 1.5 Hz, 3H), 3.15 (s, 1H), 2.79-2.63 (m, 4H), 2.04 (t, J = 11.2 Hz, 4H), 1.45 (d, J = 6.3 Hz, 3H), 1.25 (t, J = 5.5 Hz, 1H), 1.20 (t, J = 7.5 |
| | [symbol] | | | Hz, 3H). LCMS |
| | | | | m/z [M + H]+ |
| | | | | 435.29 |
| 138 | 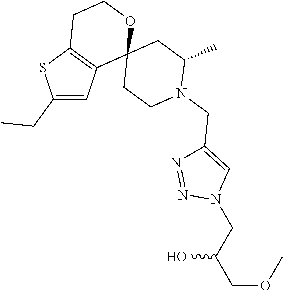 | 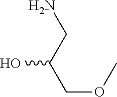 | Com- pound 15 | 1H NMR (400 MHz, DMSO-d6) δ 9.95 (s, 1H), 8.97 (s, 1H), 8.29 (d, J = 1.6 Hz, 1H), 6.46 (s, 1H), 5.40 (s, 1H), 4.63 (d, J = 14.2 Hz, 1H), 4.52 (dd, J = 13.8, 3.7 Hz, 2H), 4.37 (dd, J = 13.8, 7.7 Hz, 1H), 4.00 (s, 1H), 3.82 (h, J = 6.2, 5.8 Hz, 2H), 3.29 (s, 6H), 3.17 (s, 1H), 2.79-2.61 (m, 4H), 2.02 (d, J = 11.4 Hz, 4H), 1.45 (d, |
| | | | | J = 6.4 Hz, 3H), 1.20 |
| | | | | (t, J = 7.5 Hz, 3H). |
| | | | | LCMS m/z [M + H]+ |
| | | | | 421.25 |
| 139 | 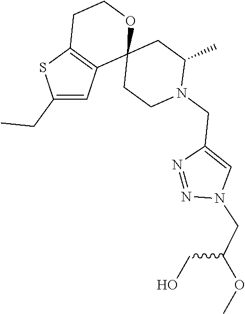 [SMILES: CCc1cc2c(s1)CCO[C@@]21CCN(Cc2cn(CC(CO)OC)nn2)[C@@H](C)C1] | 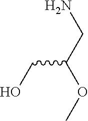 | Com- pound 15 | 1H NMR (400 MHz, DMSO-d6) δ 7.92 (s, 1H), 6.59 (s, 1H), 4.91 (t, J = 5.6 Hz, 1H), 4.54 (dd, J = 14.2, 3.8 Hz, 1H), 4.36 (dd, J = 14.1, 7.5 Hz, 1H), 3.87 (d, J = 14.7 Hz, 1H), 3.74 (h, J = 5.9 Hz, 3H), 3.57 (dd, J = 8.0, 4.2 Hz, 1H), 3.43 (td, J = 5.3, 1.8 Hz, 2H), 3.20 (s, 3H), 2.70 (q, J = 7.5 Hz, 2H), 2.66-2.58 (m, 2H), 2.57 (s, 1H), 2.44 (s, 2H), 1.78 (t, J = 11.3 Hz, |
| | | | | 1H), 1.68 (d, J = |
| | | | | 13.3 Hz, 2H), 1.55 |
| | | | | (t, J = 12.4 Hz, |
| | | | | 1H), 1.19 (t, J = 7.5 |
| | | | | Hz, 3H), 1.14 (d, |
| | | | | J = 6.1 Hz, 3H). |
| | | | | LCMS m/z [M + H]+ |
| | | | | 421.56 |
| 140 | 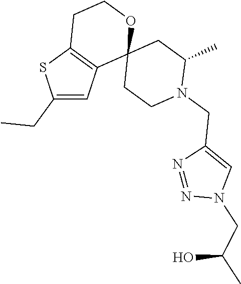 [SMILES: CCc1cc2c(s1)CCO[C@@]21CCN(Cc2cn(C[C@@H](C)O)nn2)[C@@H](C)C1] | 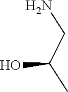 | Com- pound 15 | 1H NMR (400 MHz, DMSO-d6) δ 9.95 (s, 1H), 8.29 (s, 1H), 6.46 (s, 1H), 5.13 (s, 1H), 4.64 (d, J = 13.8 Hz, 1H), 4.52 (d, J = 12.2 Hz, 1H), 4.42 (dd, J = 13.5, 4.0 Hz, 1H), 4.36- 4.26 (m, 1H), 4.02 (d, J = 7.0 Hz, 1H), 3.96-3.73 (m, 2H), 3.30 (s, 1H), 3.17 (s, 2H), 2.80-2.61 (m, 3H), 2.28-1.83 (m, 4H), 1.52-1.34 (m, 4H), 1.30-1.16 |
| | | | | (m, 3H), 1.07 (d, |
| | | | | J = 6.2 Hz, 3H). |
| | | | | LCMS m/z [M + H]+ |
| | | | | 391.25 |
| 141 | 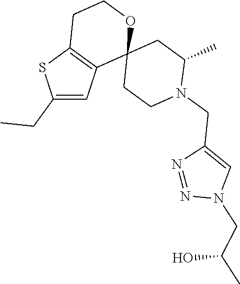 [SMILES: CCc1cc2c(s1)CCO[C@@]21CCN(Cc2cn(C[C@H](C)O)nn2)[C@@H](C)C1] | 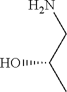 | Com- pound 15 | 1H NMR (400 MHz, DMSO-d6) δ 9.85 (s, 1H), 8.30 (s, 1H), 6.46 (d, J = 7.2 Hz, 1H), 5.13 (s, 1H), 4.64 (d, J = 13.8 Hz, 1H), 4.51 (d, J = 15.2 Hz, 1H), 4.46-4.34 (m, 2H), 4.30 (dd, J = 13.8, 7.1 Hz, 1H), 4.03 (s, 1H), 3.97- 3.73 (m, 2H), 3.24- 3.15 (m, 2H), 2.79- 2.61 (m, 3H), 2.36- 1.85 (m, 5H), 1.45 (d, J = 6.5 Hz, 3H), 1.20 (t, J = 7.5 Hz, |
| | | | | 3H), 1.08 (d, J = |
| | | | | 6.2 Hz, 3H). |
| | | | | LCMS m/z [M + H]+ |
| | | | | 391.25 |
| 142 | 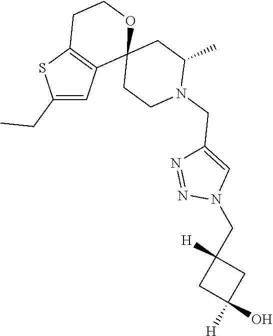 [SMILES: CCc1cc2c(s1)CCO[C@@]21CCN(Cc2cn(C[C@H]3C[C@H](O)C3)nn2)[C@@H](C)C1] | 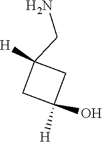 | Com- pound 15 | 1H NMR (400 MHz, DMSO-d6) δ 9.90 (s, 1H), 8.38 (s, 1H), 6.46 (d, J = 6.8 Hz, 1H), 4.62 (d, J = 13.9 Hz, 1H), 4.57-4.45 (m, 3H), 4.27-4.12 (m, 1H), 3.83 (dq, J = 19.1, 7.3, 6.4 Hz, 2H), 3.31 (d, J = 13.2 Hz, 3H), 3.15 (s, 1H), 2.81-2.57 (m, 5H), 2.03 (s, 5H), 1.98-1.87 (m, 3H), 1.44 (d, J = 6.4 Hz, 3H), 1.20 (td, J = 7.5, 3.7 Hz, 3H). LCMS m/z [M + H]+ 417.27 |
| 143 | 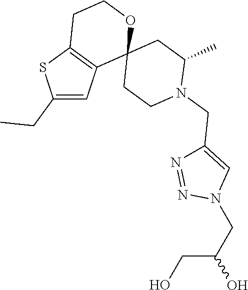 | 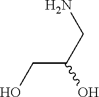 | Com- pound 15 | 1H NMR (400 MHz, DMSO-d6) δ 8.97 (s, 2H), 8.29 (s, 1H), 6.46 (s, 1H), 5.23 (s, 1H), 4.91 (s, 1H), 4.57 (td, J = 23.8, 23.0, 14.1 Hz, 5H), 4.40- 4.26 (m, 2H), 3.84 (s, 4H), 2.80-2.62 (m, 4H), 2.02 (d, J = 10.7 Hz, 4H), 1.45 (d, J = 6.4 Hz, 3H), 1.20 (t, J = 7.5 Hz, 3H). LCMS m/z [M + H]+ 407.24 |
| 144 | 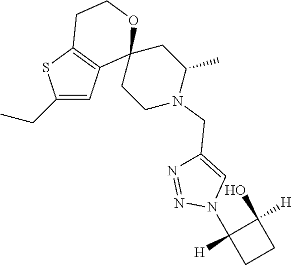 [SMILES: CCc1cc2c(s1)CCO[C@@]21CCN(Cc2cn([C@H]3CC[C@@H]3O)nn2)[C@@H](C)C1] | 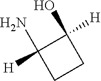 | Com- pound 15 | 1H NMR (400 MHz, DMSO-d6) δ 9.88 (s, 1H), 8.49 (s, 1H), 6.45 (s, 1H), 5.86 (s, 1H), 4.82 (q, J = 8.6 Hz, 1H), 4.63 (d, J = 14.0 Hz, 1H), 4.51 (s, 1H), 4.37 (d, J = 8.9 Hz, 1H), 3.94- 3.66 (m, 3H), 3.19 (d, J = 14.7 Hz, 1H), 2.80-2.64 (m, 4H), 2.21 (dp, J = 27.0, 9.0 Hz, 3H), 2.02 (d, J = 12.1 Hz, 3H), 1.92 (p, |
| | | | | J = 10.0, 9.5 Hz, |
| | | | | 2H), 1.68 (q, J = |
| | | | | 9.6 Hz, 1H), 1.45 |
| | | | | (d, J = 6.4 Hz, 3H), |
| | | | | 1.20 (t, J = 7.5 Hz, |
| | | | | 3H). LCMS m/z |
| | | | | [M + H]+ 403.23 |
| 145 | 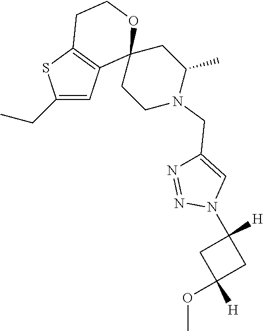 [SMILES: CCc1cc2c(s1)CCO[C@@]21CCN(Cc2cn([C@H]3C[C@@H](OC)C3)nn2)[C@@H](C)C1] | 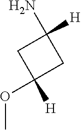 | Com- pound 15 | 1H NMR (400 MHz, DMSO-d6) δ 9.98 (s, 1H), 8.49 (s, 1H), 6.50 (d, J = 33.9 Hz, 1H), 4.87 (p, J = 8.3 Hz, 1H), 4.62 (d, J = 14.1 Hz, 1H), 4.48 (d, J = 15.4 Hz, 1H), 3.91-3.76 (m, 3H), 3.21 (s, 5H), 2.97- 2.88 (m, 2H), 2.86- 2.58 (m, 4H), 2.44- 2.34 (m, 2H), 2.18- 1.90 (m, 4H), 1.44 (d, J = 6.4 Hz, 3H), 1.37 (d, J = 6.4 Hz, 1H), 1.31-1.11 (m, 3H). LCMS m/z [M + H]+ 417.27 |
| 146 | 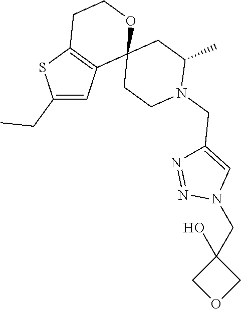 [SMILES: CCc1cc2c(s1)CCO[C@@]21CCN(Cc2cn(CC3(O)COC3)nn2)[C@@H](C)C1] | 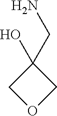 | Com- pound 15 | 1H NMR (400 MHz, DMSO-d6) δ 9.97 (s, 1H), 8.31 (d, J = 2.8 Hz, 1H), 6.42 (d, J = 31.7 Hz, 1H), 4.77 (s, 3H), 4.64 (d, J = 13.7 Hz, 1H), 4.60- 4.37 (m, 6H), 3.83 (dt, J = 13.6, 7.1 Hz, 2H), 3.27- 3.10 (m, 2H), 2.78- 2.59 (m, 4H), 2.26- 1.83 (m, 4H), 1.45 (t, J = 7.0 Hz, 3H), 1.25-1.14 (m, 3H). LCMS m/z [M + H]+ 419.23 |
| 147 | 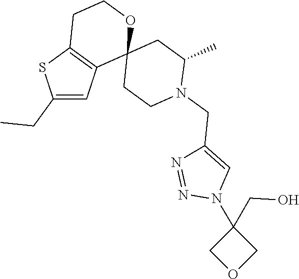 [SMILES: CCc1cc2c(s1)CCO[C@@]21CCN(Cc2cn(C3(CO)COC3)nn2)[C@@H](C)C1] | 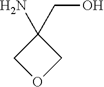 | Com- pound 15 | 1H NMR (400 MHz, DMSO-d6) δ 10.02 (s, 1H), 8.49 (d, J = 3.0 Hz, 1H), 6.47 (s, 1H), 5.80- 5.56 (m, 1H), 5.00 (dd, J = 6.9, 4.3 Hz, 2H), 4.83 (d, J = 7.0 Hz, 2H), 4.67 (d, J = 14.1 Hz, 1H), 4.51 (d, J = 13.5 Hz, 1H), 4.06 (s, 2H), 3.97-3.78 (m, 2H), 3.35-3.06 (m, 2H), 2.92-2.59 (m, 4H), 2.24-1.89 (m, 4H), 1.46 (t, J = 7.0 Hz, 3H), 1.31- |
| | | | | 1.15 (m, 3H). |
| | | | | LCMS m/z [M + H]+ |
| | | | | 419.23 |
| 148 | 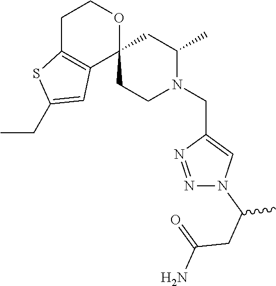 | 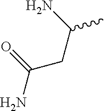 | Com- pound 15 | 1H NMR (400 MHz, DMSO-d6) δ 9.93 (s, 1H), 8.36 (d, J = 2.1 Hz, 1H), 7.46 (s, 1H), 6.92 (s, 1H), 6.46 (s, 1H), 5.10 (q, J = 7.1 Hz, 1H), 4.62 (d, J = 14.2 Hz, 1H), 4.48 (d, J = 15.1 Hz, 1H), 3.82 (dq, J = 11.9, 5.6 Hz, 2H), 2.75 (tdd, J = 22.0, 12.7, 6.0 Hz, 7H), 2.08 (dd, J = 49.0, 13.0 Hz, 4H), 1.53 (d, J = 6.8 Hz, 4H), 1.44 |
| | | | | (d, J = 6.5 Hz, 3H), |
| | | | | 1.38 (d, J = 7.4 Hz, |
| | | | | 1H), 1.21 (q, J = |
| | | | | 7.7 Hz, 3H). |
| | | | | LCMS m/z [M + H]+ |
| | | | | 418.28 |
| 149 | 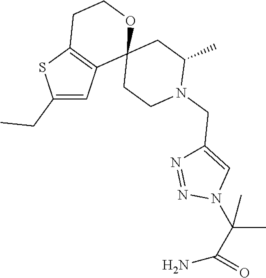 [SMILES: CCc1cc2c(s1)CCO[C@@]21CCN(Cc2cn(C(C)(C)C(N)=O)nn2)[C@@H](C)C1] | 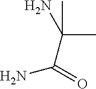 | Com- pound 15 | 1H NMR (400 MHz, DMSO-d6) δ 9.94 (s, 1H), 8.44 (d, J = 3.3 Hz, 1H), 7.45 (d, J = 6.6 Hz, 2H), 6.47 (d, J = 7.1 Hz, 1H), 4.71- 4.39 (m, 2H), 3.84 (dt, J = 14.1, 6.4 Hz, 2H), 3.21 (s, 2H), 2.87-2.65 (m, 4H), 2.27-1.90 (m, 4H), 1.85 (s, 6H), 1.46 (t, J = 6.7 Hz, 3H), 1.41-1.25 (m, 1H), 1.20 (td, J = 7.5, 3.0 Hz, 3H). LCMS m/z [M + H]+ |
| | | | | 418.25 |
| 150 | 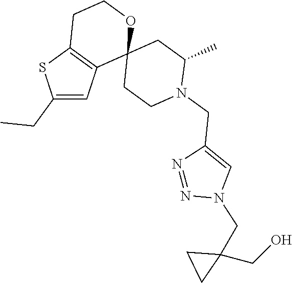 [SMILES: CCc1cc2c(s1)CCO[C@@]21CCN(Cc2cn(CC3(CO)CC3)nn2)[C@@H](C)C1] | 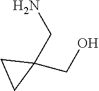 | Com- pound 15 | 1H NMR (400 MHz, DMSO-d6) δ 10.06 (d, J = 39.7 Hz, 1H), 8.36 (s, 1H), 6.46 (s, 1H), 4.98-4.25 (m, 6H), 3.81 (d, J = 6.5 Hz, 2H), 3.31 (d, J = 12.3 Hz, 3H), 3.13 (s, 3H), 2.79-2.61 (m, 2H), 2.06 (dq, J = 27.6, 17.4, 15.5 Hz, 4H), 1.49- 1.34 (m, 3H), 1.32- 1.09 (m, 3H), 0.66 (d, J = 4.4 Hz, 2H), 0.52 (s, 2H). LCMS m/z [M + H]+ |
| | | | | 417.24 |
| 151 | 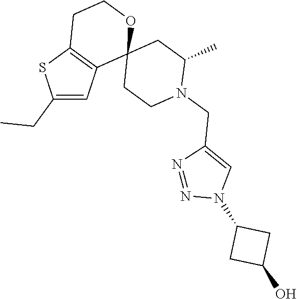 [SMILES: CCc1cc2c(s1)CCO[C@@]21CCN(Cc2cn([C@H]3C[C@H](O)C3)nn2)[C@@H](C)C1] | 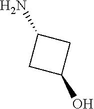 | Com- pound 15 | 1H NMR (400 MHz, DMSO-d6) δ 10.04 (d, J = 46.6 Hz, 1H), 8.46 (d, J = 2.7 Hz, 1H), 6.47 (d, J = 7.7 Hz, 1H), 5.42 (s, 1H), 5.25 (dq, J = 8.6, 4.8, 4.1 Hz, 1H), 4.62 (d, J = 14.1 Hz, 1H), 4.57-4.47 (m, 2H), 3.84 (dd, J = 15.5, 9.5 Hz, 2H), 3.18 (s, 2H), 2.88 (dd, J = 31.0, 6.4 Hz, 1H), 2.79- 2.64 (m, 5H), 2.15 (d, J = 14.4 Hz, 1H), 2.02 (d, J = |
| | | | | 17.3 Hz, 3H), 1.45 |
| | | | | (t, J = 6.8 Hz, 3H), |
| | | | | 1.40-1.23 (m, 2H), |
| | | | | 1.32-1.15 (m, 3H). |
| | | | | LCMS m/z [M + H]+ |
| | | | | 403.27 |
| 152 | 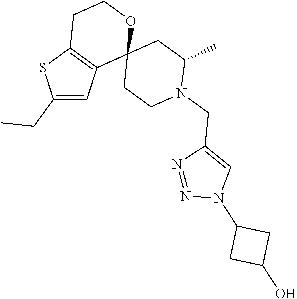 [SMILES: CCc1cc2c(s1)CCO[C@@]21CCN(Cc2cn(C3CC(O)C3)nn2)[C@@H](C)C1] | 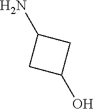 | Com- pound 15 | 1H NMR (400 MHz, DMSO-d6) δ 9.99 (s, 1H), 8.45 (s, 1H), 6.47 (d, J = 7.1 Hz, 1H), 5.44 (s, 1H), 4.73 (q, J = 8.4 Hz, 1H), 4.62 (d, J = 14.0 Hz, 1H), 4.44 (d, J = 49.1 Hz, 2H), 4.05 (p, J = 7.1 Hz, 1H), 3.83 (tt, J = 12.4, 5.7 Hz, 2H), 3.18 (s, 2H), 2.94-2.80 (m, 2H), 2.72 (dt, J = 21.9, 6.9 Hz, 4H), 2.42-2.32 (m, 2H), 2.02 (d, J = 15.9 Hz, 4H), 1.45 |
| | | | | (d, J = 6.5 Hz, 3H), |
| | | | | 1.20 (t, J = 7.5 Hz, |
| | | | | 3H). LCMS m/z |
| | | | | [M + H]+ 403.23 |
| 153 | 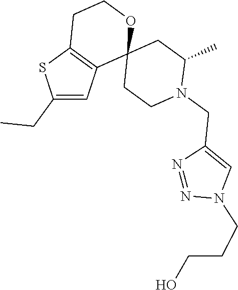 [SMILES: CCc1cc2c(s1)CCO[C@@]21CCN(Cc2cn(CCCO)nn2)[C@@H](C)C1] | 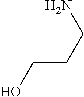 | Com- pound 15 | 1H NMR (400 MHz, DMSO-d6) δ 10.00 (s, 1H), 8.37 (d, J = 2.2 Hz, 1H), 6.46 (s, 1H), 4.62 (d, J = 13.9 Hz, 1H), 4.49 (t, J = 7.1 Hz, 4H), 3.82 (d, J = 6.7 Hz, 3H), 3.17 (s, 2H), 2.80-2.62 (m, 4H), 2.09-1.91 (m, 7H), 1.45 (t, J = 7.0 Hz, 4H), 1.20 (t, J = 7.5 Hz, 3H). LCMS m/z [M + H]+ 391.25 |
| 154 | 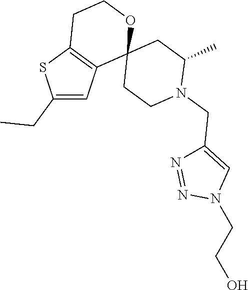 | 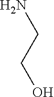 | Com- pound 15 | 1H NMR (400 MHz, DMSO-d6) δ 10.02 (d, J = 39.0 Hz, 1H), 8.33 (s, 1H), 6.46 (s, 1H), 5.11 (s, 1H), 4.63 (d, J = 14.0 Hz, 1H), 4.58-4.42 (m, 4H), 3.90-3.74 (m, 5H), 3.18 (d, J = 12.0 Hz, 1H), 2.78- 2.62 (m, 4H), 2.04 (s, 4H), 1.41 (d, J = 25.5 Hz, 3H), 1.27- 1.17 (m, 3H). LCMS m/z [M + H]+ 377.04 |
| 155 | 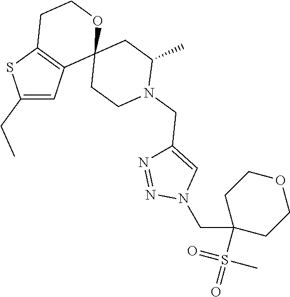 [SMILES: CCc1cc2c(s1)CCO[C@@]21CCN(Cc2cn(CC3(S(C)(=O)=O)CCOCC3)nn2)[C@@H](C)C1] | 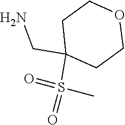 | Com- pound 15 | 1H NMR (300 MHz, Chloroform- d) δ 8.31 (s, 1H), 6.45 (d, J = 1.1 Hz, 1H), 5.08 (s, 2H), 4.77 (d, J = 14.2 Hz, 1H), 4.38 (d, J = 14.2 Hz, 1H), 4.17-4.08 (m, 2H), 3.93-3.79 (m, 4H), 3.55 (s, 1H), 3.29 (d, J = 13.4 Hz, 2H), 2.83 (s, 3H), 2.81-2.71 (m, 4H), 2.33-2.18 (m, 4H), 2.07-1.99 (m, 2H), 1.87-1.77 (m, 2H), 1.61 (s, 3H), 1.27 (t, J = 7.5 Hz, 3H). |
| | | | | LCMS m/z [M + H]+ |
| | | | | 509.295 |
| 156 | 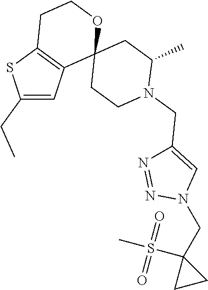 [SMILES: CCc1cc2c(s1)CCO[C@@]21CCN(Cc2cn(CC3(S(C)(=O)=O)CC3)nn2)[C@@H](C)C1] | 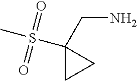 | Com- pound 15 | 1H NMR (300 MHz, Chloroform- d) δ 8.39 (s, 1H), 6.45 (d, J = 1.1 Hz, 1H), 4.83 (d, J = 15.0 Hz, 3H), 4.32 (d, J = 14.0 Hz, 1H), 3.93-3.81 (m, 2H), 3.60 (s, 1H), 3.30 (q, J = 13.3, 10.3 Hz, 2H), 2.79 (s, 3H), 2.76 (t, J = 7.4 Hz, 4H), 2.35- 2.19 (m, 2H), 2.09- 1.98 (m, 2H), 1.72- 1.66 (m, 2H), 1.60 (s, 3H), 1.41-1.36 (m, 2H), 1.27 (t, J = |
| | | | | 7.5 Hz, 3H). |
| | | | | LCMS m/z [M + H]+ |
| | | | | 465.32 |
| 157 | 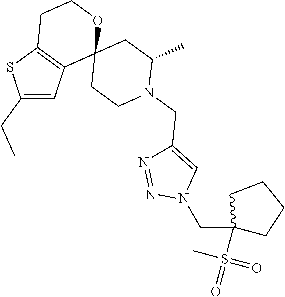 [SMILES: CCc1cc2c(s1)CCO[C@@]21CCN(Cc2cn(CC3(S(C)(=O)=O)CCCC3)nn2)[C@@H](C)C1] | 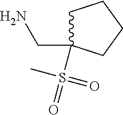 | Com- pound 15 | 1H NMR (300 MHz, Chloroform- d) δ 8.32 (s, 1H), 6.47 (d, J = 1.2 Hz, 1H), 4.78 (d, J = 14.3 Hz, 3H), 4.33 (d, J = 14.1 Hz, 2H), 3.93-3.80 (m, 2H), 3.55 (s, 1H), 3.27 (d, J = 15.4 Hz, 2H), 2.80 (s, 3H), 2.75 (dd, J = 10.0, 5.6 Hz, 4H), 2.40-2.24 (m, 4H), 2.05 (ddt, J = 18.9, 12.9, 6.4 Hz, 4H), 1.85 (d, J = 6.9 Hz, 2H), 1.74-1.61 (m, 2H), 1.61 (d, J = |
| | | | | 6.5 Hz, 3H), 1.27 |
| | | | | (t, J = 7.5 Hz, 3H). |
| | | | | LCMS m/z [M + H]+ |
| | | | | 493.21 |
| 158 | 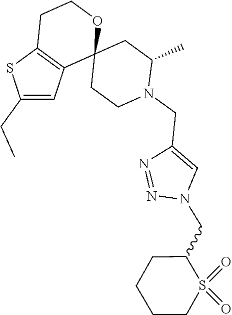 [SMILES: CCc1cc2c(s1)CCO[C@@]21CCN(Cc2cn(CC3CCCCS3(=O)=O)nn2)[C@@H](C)C1] | 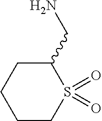 | Com- pound 15 | 1H NMR (300 MHz, Chloroform- d) δ 8.24 (d, J = 10.1 Hz, 1H), 6.46 (d, J = 1.2 Hz, 1H), 5.04 (ddd, J = 14.6, 8.6, 7.1 Hz, 1H), 4.64 (ddd, J = 14.4, 8.0, 6.1 Hz, 2H), 4.34 (dd, J = 14.1, 7.8 Hz, 1H), 3.86 (ddd, J = 15.4, 6.6, 4.5 Hz, 2H), 3.57 (d, J = 6.0 Hz, 2H), 3.32 (s, 2H), 3.16 (dd, J = 14.5, 4.2 Hz, 1H), 3.06- 2.93 (m, 1H), 2.77 (q, J = 7.1 Hz, 4H), 2.35-2.19 (m, 2H), |
| | | | | 2.15-1.98 (m, 5H), |
| | | | | 1.95-1.81 (m, 2H), |
| | | | | 1.60 (dd, J = 6.5, |
| | | | | 5.0 Hz, 3H), 1.52 |
| | | | | (d, J = 10.6 Hz, |
| | | | | 2H), 1.27 (t, J = 7.5 |
| | | | | Hz, 3H). LCMS |
| | | | | m/z [M + H]+ |
| | | | | 479.04 |
| 159 | 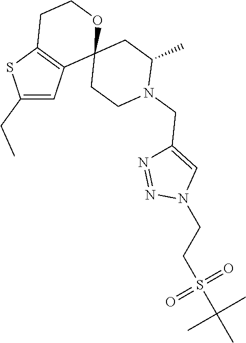 [SMILES: CCc1cc2c(s1)CCO[C@@]21CCN(Cc2cn(CCS(=O)(=O)C(C)(C)C)nn2)[C@@H](C)C1] | 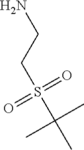 | Com- pound 15 | 1H NMR (300 MHz, Chloroform- d) δ 8.25 (s, 1H), 6.45 (d, J = 1.3 Hz, 1H), 5.01 (td, J = 6.8, 2.9 Hz, 2H), 4.77 (d, J = 14.1 Hz, 1H), 4.34 (d, J = 14.1 Hz, 1H), 3.95-3.78 (m, 2H), 3.60 (t, J = 6.8 Hz, 3H), 3.31 (d, J = 14.9 Hz, 2H), 2.85- 2.69 (m, 4H), 2.34- 2.19 (m, 2H), 2.03 (ddd, J = 12.6, 6.3, 3.2 Hz, 2H), 1.60 (d, J = 6.5 Hz, 3H), 1.44 (s, 9H), 1.27 (t, J = 7.5 Hz, 3H). LCMS m/z [M + H]+ 481.22 |
| 160 | 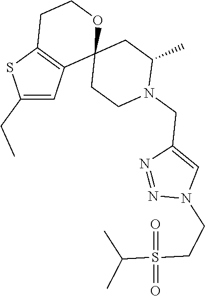 [SMILES: CCc1cc2c(s1)CCO[C@@]21CCN(Cc2cn(CCS(=O)(=O)C(C)C)nn2)[C@@H](C)C1] | 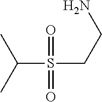 | Com- pound 15 | 1H NMR (300 MHz, Chloroform- d) δ 8.27 (s, 1H), 6.46 (d, J = 1.1 Hz, 1H), 4.99 (td, J = 6.7, 2.7 Hz, 2H), 4.78 (d, J = 14.1 Hz, 1H), 4.31 (d, J = 14.0 Hz, 1H), 3.96-3.80 (m, 2H), 3.65-3.54 (m, 3H), 3.29 (dd, J = 20.1, 7.8 Hz, 2H), 3.13 (p, J = 6.9 Hz, 1H), 2.84-2.71 (m, 4H), 2.35-2.19 (m, 2H), 2.09-1.97 (m, 2H), 1.60 (d, J = 6.5 Hz, |
| | | | | 3H), 1.41 (dd, J = |
| | | | | 6.9, 1.4 Hz, 6H), |
| | | | | 1.27 (t, J = 7.5 Hz, |
| | | | | 3H). LCMS m/z |
| | | | | [M + H]+ 467.18 |
| 161 | 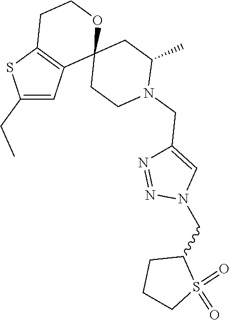 [SMILES: CCc1cc2c(s1)CCO[C@@]21CCN(Cc2cn(CC3CCCS3(=O)=O)nn2)[C@@H](C)C1] | 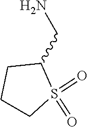 | Com- pound 15 | 1H NMR (300 MHz, Chloroform- d) δ 8.25 (s, 1H), 6.46 (d, J = 1.8 Hz, 1H), 4.89 (ddd, J = 14.6, 8.3, 2.4 Hz, 1H), 4.80-4.70 (m, 2H), 4.41-4.29 (m, 1H), 3.95-3.79 (m, 2H), 3.63 (p, J = 7.5 Hz, 2H), 3.37- 3.19 (m, 3H), 3.10 (tdd, J = 10.8, 7.7, 3.9 Hz, 1H), 2.77 (td, J = 7.7, 5.9 Hz, 4H), 2.51-2.40 (m, 1H), 2.35-2.15 (m, 4H), 2.08-1.92 (m, 3H), 1.60 (t, J = 6.3 Hz, 3H), 1.27 (t, |
| | | | | J = 7.5 Hz, 3H). |
| | | | | LCMS m/z [M + H]+ |
| | | | | 465.16 |
| 162 | 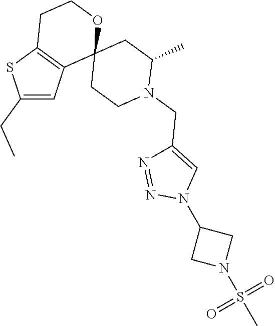 [SMILES: CCc1cc2c(s1)CCO[C@@]21CCN(Cc2cn(C3CN(S(C)(=O)=O)C3)nn2)[C@@H](C)C1] | 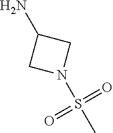 | Com- pound 15 | 1H NMR (400 MHz, Chloroform- d) δ 7.71 (s, 1H), 6.49 (d, J = 1.1 Hz, 1H), 5.40 (tt, J = 7.8, 6.1 Hz, 1H), 4.58-4.38 (m, 4H), 4.12-4.06 (m, 1H), 3.96-3.81 (m, 3H), 3.06 (s, 3H), 2.84- 2.55 (m, 7H), 1.97- 1.82 (m, 3H), 1.77- 1.68 (m, 1H), 1.29 (t, J = 7.5 Hz, 3H), 1.25 (d, J = 6.2 Hz, 3H). [2] LCMS m/z [M + H]+ 466.315 |
| 163 | 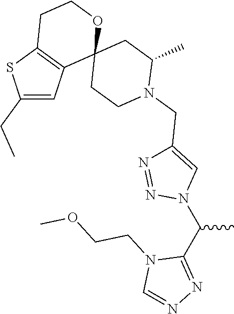 [SMILES: CCc1cc2c(s1)CCO[C@@]21CCN(Cc2cn(C(C)c3nncn3CCOC)nn2)[C@@H](C)C1] | 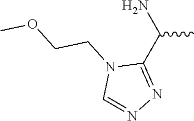 | Com- pound 15 | LCMS m/z [M + H]+ 486.27 |
| 164 | 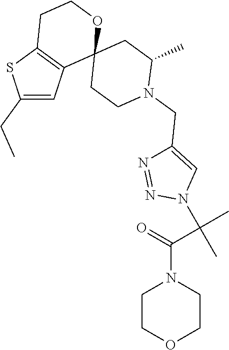 [SMILES: CCc1cc2c(s1)CCO[C@@]21CCN(Cc2cn(C(C)(C)C(=O)N3CCOCC3)nn2)[C@@H](C)C1] | 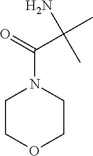 | Com- pound 15 | LCMS m/z [M + H]+ 488.29 |
| 165 | 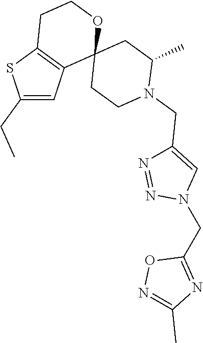 [SMILES: CCc1cc2c(s1)CCO[C@@]21CCN(Cc2cn(Cc3nc(C)no3)nn2)[C@@H](C)C1] | 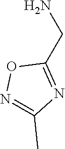 | Com- pound 15 | 1H NMR (400 MHz, DMSO-d6) δ 10.17 (s, 1H), 8.56 (s, 1H), 6.47 (s, 1H), 6.21 (s, 2H), 4.69 (d, J = 14.3 Hz, 1H), 4.59 (d, J = 14.5 Hz, 1H), 3.89-3.76 (m, 2H), 3.62 (d, J = 3.8 Hz, 1H), 3.32 (s, 2H), 3.23-3.06 (m, 2H), 2.68 (s, 2H), 2.34 (s, 3H), 2.06 (t, J = 11.1 Hz, 4H), 1.46 (d, J = 6.3 Hz, 3H), 1.20 (t, J = 7.6 Hz, 3H). [2] LCMS m/z [M + H]+ 429.215 |
| 166 | 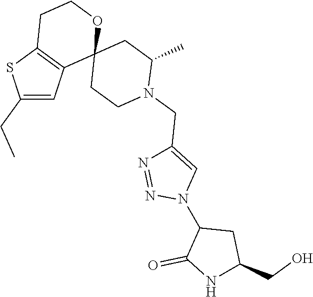 [SMILES: CCc1cc2c(s1)CCO[C@@]21CCN(Cc2cn(C3C[C@@H](CO)NC3=O)nn2)[C@@H](C)C1] | 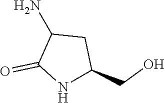 | Com- pound 15 | LCMS m/z [M + H]+ 446.2 |
| 167 | 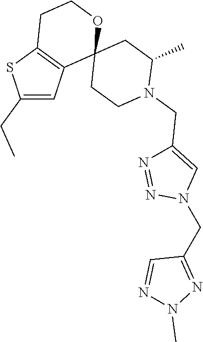 [SMILES: CCc1cc2c(s1)CCO[C@@]21CCN(Cc2cn(Cc3cnn(C)n3)nn2)[C@@H](C)C1] | 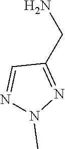 | Com- pound 15 | LCMS m/z [M + H]+ 428.38 |
| 168 | 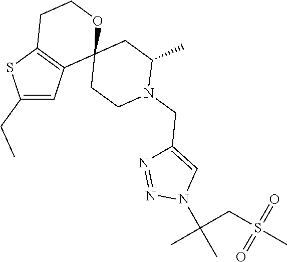 [SMILES: CCc1cc2c(s1)CCO[C@@]21CCN(Cc2cn(C(C)(C)CS(C)(=O)=O)nn2)[C@@H](C)C1] | 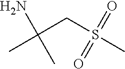 | Com- pound 15 | LCMS m/z [M + H]+ 467.21 |
| 169 | 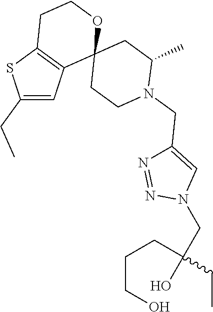 [SMILES: CCc1cc2c(s1)CCO[C@@]21CCN(Cc2cn(CC(O)(CC)CCCO)nn2)[C@@H](C)C1] | 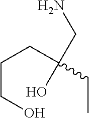 | Com- pound 15 | LCMS m/z [M + H]+ 463.27 |
| 170 | 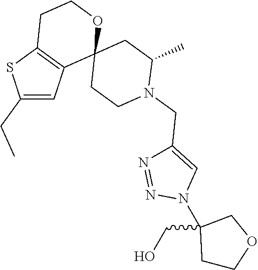 [SMILES: CCc1cc2c(s1)CCO[C@@]21CCN(Cc2cn(C3(CO)CCOC3)nn2)[C@@H](C)C1] | 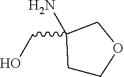 | Com- pound 15 | LCMS m/z [M + H]+ 433.24 |
| 171 | 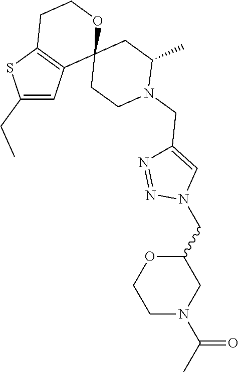 [SMILES: CCc1cc2c(s1)CCO[C@@]21CCN(Cc2cn(CC3CN(C(C)=O)CCO3)nn2)[C@@H](C)C1] | 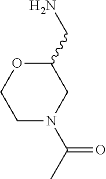 | Com- pound 15 | LCMS m/z [M + H]+ 474.25 |
| 172 | 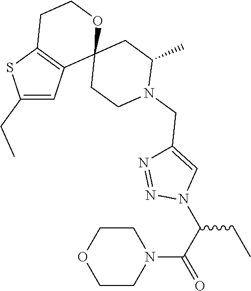 [SMILES: CCc1cc2c(s1)CCO[C@@]21CCN(Cc2cn(C(CC)C(=O)N3CCOCC3)nn2)[C@@H](C)C1] | 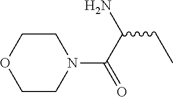 | Com- pound 15 | LCMS m/z [M + H]+ 488.25 |
| 173 | 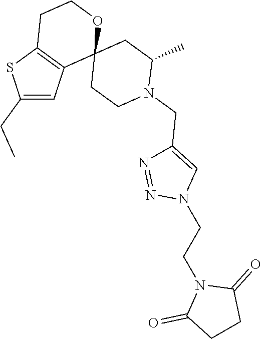 [SMILES: CCc1cc2c(s1)CCO[C@@]21CCN(Cc2cn(CCN3C(=O)CCC3=O)nn2)[C@@H](C)C1] | 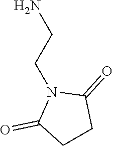 | Com- pound 15 | LCMS m/z [M + H]+ 458.22 |
| 174 | 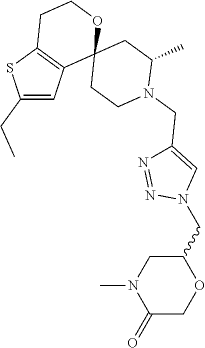 [SMILES: CCc1cc2c(s1)CCO[C@@]21CCN(Cc2cn(CC3CN(C)C(=O)CO3)nn2)[C@@H](C)C1] | 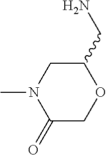 | Com- pound 15 | LCMS m/z [M + H]+ 460.21 |
| 175 | 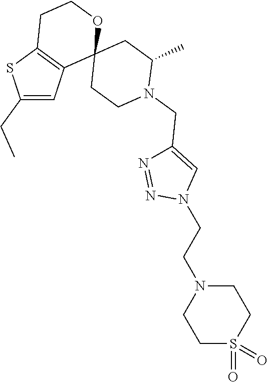 [SMILES: CCc1cc2c(s1)CCO[C@@]21CCN(Cc2cn(CCN3CCS(=O)(=O)CC3)nn2)[C@@H](C)C1] | 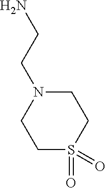 [SMILES: NCCN1CCS(=O)(=O)CC1] | Com- pound 15 | LCMS m/z [M + H]+ 494.05 |
| 176 | 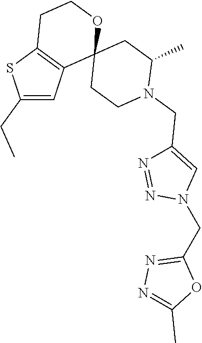 [SMILES: CCc1cc2c(s1)CCO[C@@]21CCN(Cc2cn(Cc3nnc(C)o3)nn2)[C@@H](C)C1] | 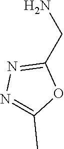 | Com- pound 15 | LCMS m/z [M + H]+ 429.2 |
| 177 | 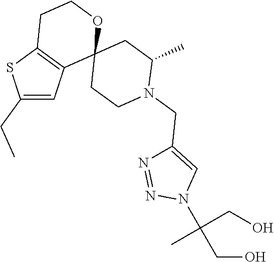 [SMILES: CCc1cc2c(s1)CCO[C@@]21CCN(Cc2cn(C(C)(CO)CO)nn2)[C@@H](C)C1] | 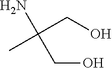 | Com- pound 15 | LCMS m/z [M + H]+ 421.21 |
| 178 | 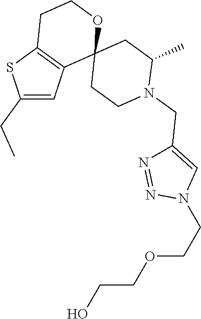 [SMILES: CCc1cc2c(s1)CCO[C@@]21CCN(Cc2cn(CCOCCO)nn2)[C@@H](C)C1] | 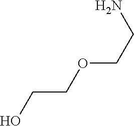 | Com- pound 15 | LCMS m/z [M + H]+ 421.08 |
| 179 | 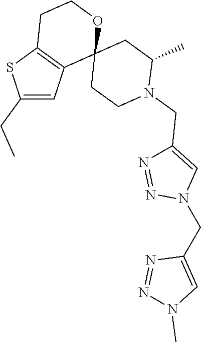 [SMILES: CCc1cc2c(s1)CCO[C@@]21CCN(Cc2cn(Cc3cn(C)nn3)nn2)[C@@H](C)C1] | 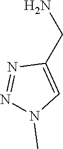 | Com- pound 15 | LCMS m/z [M + H]+ 428.25 |
| 180 | 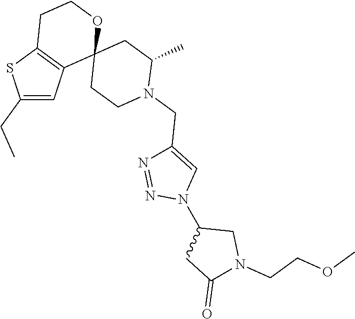 [SMILES: CCc1cc2c(s1)CCO[C@@]21CCN(Cc2cn(C3CC(=O)N(CCOC)C3)nn2)[C@@H](C)C1] | 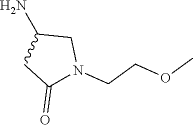 | Com- pound 15 | LCMS m/z [M + H]+ 474.28 |
| 181 | 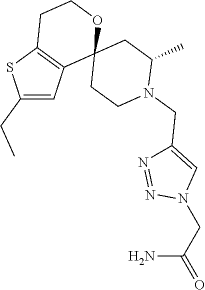 [SMILES: CCc1cc2c(s1)CCO[C@@]21CCN(Cc2cn(CC(N)=O)nn2)[C@@H](C)C1] | 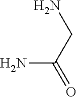 | Com- pound 15 | LCMS m/z [M + H]+ 390.04 |
| 182 | 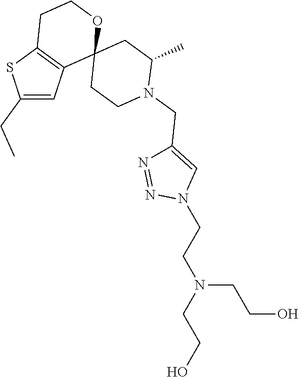 [SMILES: CCc1cc2c(s1)CCO[C@@]21CCN(Cc2cn(CCN(CCO)CCO)nn2)[C@@H](C)C1] | 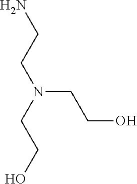 | Com- pound 15 | LCMS m/z [M + H]+ 464.25 |
| 183 | 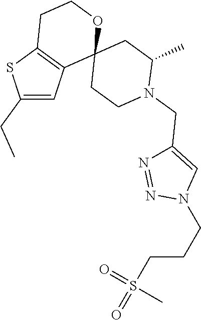 [SMILES: CCc1cc2c(s1)CCO[C@@]21CCN(Cc2cn(CCCS(C)(=O)=O)nn2)[C@@H](C)C1] | 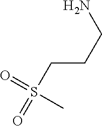 | Com- pound 15 | LCMS m/z [M + H]+ 453.2 |
| 184 | 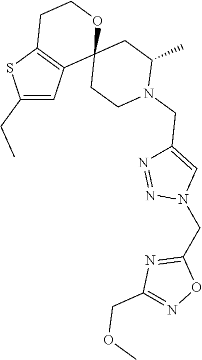 [SMILES: CCc1cc2c(s1)CCO[C@@]21CCN(Cc2cn(Cc3nc(COC)no3)nn2)[C@@H](C)C1] | 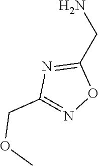 | Com- pound 15 | 1H NMR (400 MHz, DMSO-d6) δ 10.19 (s, 1H), 8.57 (s, 1H), 6.47 (s, 1H), 6.27 (s, 2H), 5.52 (s, 1H), 4.74- 4.55 (m, 3H), 4.54 (s, 3H), 3.95-3.75 (m, 3H), 3.73 (s, 1H), 3.61 (d, J = 5.9 Hz, 1H), 3.16 (s, 2H), 2.68 (s, 2H), 2.06 (t, J = 11.1 Hz, 4H), 1.45 (d, J = 6.5 Hz, 3H), 1.20 (t, J = 7.6 Hz, 3H). [2] LCMS m/z [M + H]+ 459.17 |
| 185 | 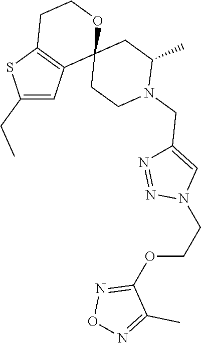 [SMILES: CCc1cc2c(s1)CCO[C@@]21CCN(Cc2cn(CCOc3nonc3C)nn2)[C@@H](C)C1] | 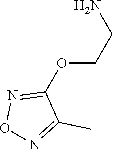 | Com- pound 15 | 1H NMR (400 MHz, DMSO-d6) δ 10.15 (s, 1H), 8.46 (s, 1H), 6.47 (s, 1H), 4.93 (t, J = 4.7 Hz, 2H), 4.77 (t, J = 4.8 Hz, 2H), 4.63 (d, J = 14.1 Hz, 1H), 4.54 (d, J = 14.7 Hz, 1H), 3.78 (h, J = 6.1 Hz, 2H), 3.62 (d, J = 3.9 Hz, 1H), 3.27 (d, J = 12.3 Hz, 2H), 3.22- 3.02 (m, 1H), 2.79- 2.62 (m, 3H), 2.18 (s, 3H), 2.13-1.93 (m, 3H), 1.44 (d, J = 6.4 Hz, 3H), 1.25 (q, J = 7.3, 6.7 Hz, 2H), 1.20 (t, J = 7.5 |
| | | | | Hz, 2H). [2] LCMS |
| | | | | m/z [M + H]+ |
| | | | | 459.165 |
| 186 | 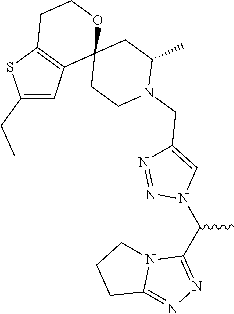 [SMILES: CCc1cc2c(s1)CCO[C@@]21CCN(Cc2cn(C(C)c3nnc4n3CCC4)nn2)[C@@H](C)C1] | 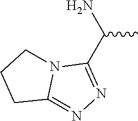 | Com- pound 15 | LCMS m/z [M + H]+ 468.25 |
| 187 | 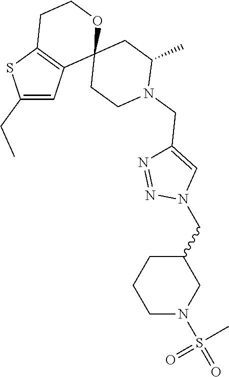 [SMILES: CCc1cc2c(s1)CCO[C@@]21CCN(Cc2cn(CC3CCCN(S(C)(=O)=O)C3)nn2)[C@@H](C)C1] | 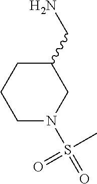 | Com- pound 15 | LCMS m/z [M + H]+ 508.22 |
| 188 | 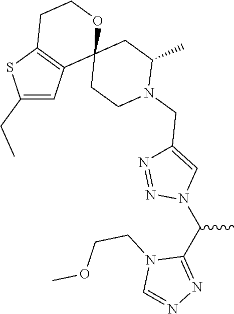 | 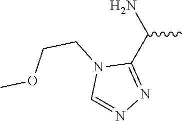 | Com- pound 15 | LCMS m/z [M + H]+ 500.31 |
| 189 | 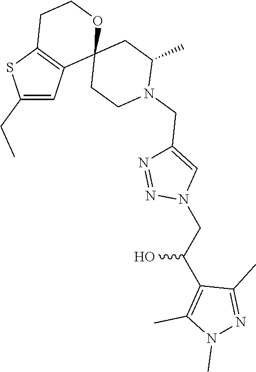 [SMILES: CCc1cc2c(s1)CCO[C@@]21CCN(Cc2cn(CC(O)c3c(C)nn(C)c3C)nn2)[C@@H](C)C1] | 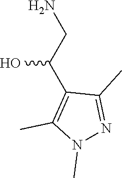 | Com- pound 15 | LCMS m/z [M + H]+ 485.29 |
| 190 | 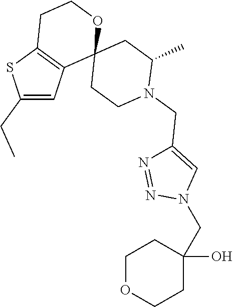 [SMILES: CCc1cc2c(s1)CCO[C@@]21CCN(Cc2cn(CC3(O)CCOCC3)nn2)[C@@H](C)C1] | 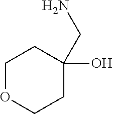 | Com- pound 15 | LCMS m/z [M + H]+ 447.24 |
| 191 | 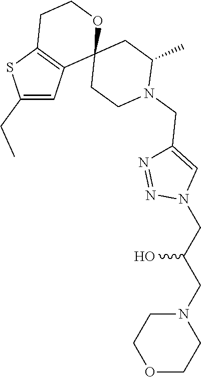 [SMILES: CCc1cc2c(s1)CCO[C@@]21CCN(Cc2cn(CC(O)CN3CCOCC3)nn2)[C@@H](C)C1] | 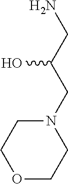 [SMILES: NCC(O)CN1CCOCC1] | Com- pound 15 | LCMS m/z [M + H]+ 476.3 |
| 192 | 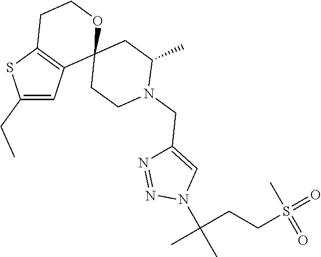 [SMILES: CCc1cc2c(s1)CCO[C@@]21CCN(Cc2cn(C(C)(C)CCS(C)(=O)=O)nn2)[C@@H](C)C1] | 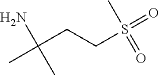 | Com- pound 15 | LCMS m/z [M + H]+ 481.22 |
| 193 | 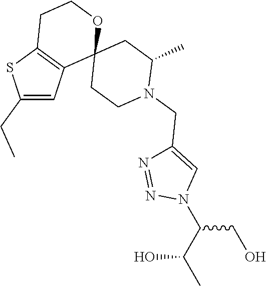 [SMILES: CCc1cc2c(s1)CCO[C@@]21CCN(Cc2cn(C(CO)[C@H](C)O)nn2)[C@@H](C)C1] | 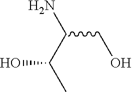 | Com- pound 15 | LCMS m/z [M + H]+ 421.25 |
| 194 | 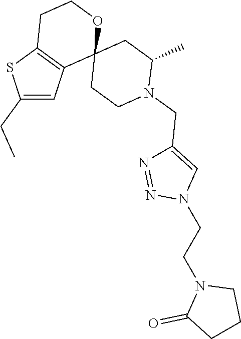 [SMILES: CCc1cc2c(s1)CCO[C@@]21CCN(Cc2cn(CCN3CCCC3=O)nn2)[C@@H](C)C1] | 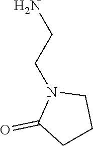 | Com- pound 15 | LCMS m/z [M + H]+ 444.25 |
| 195 | 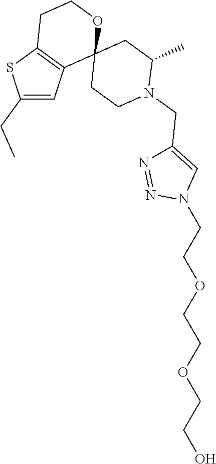 [SMILES: CCc1cc2c(s1)CCO[C@@]21CCN(Cc2cn(CCOCCOCCO)nn2)[C@@H](C)C1] | 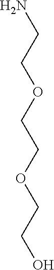 | Com- pound 15 | LCMS m/z [M + H]+ 465.26 |
| 196 | 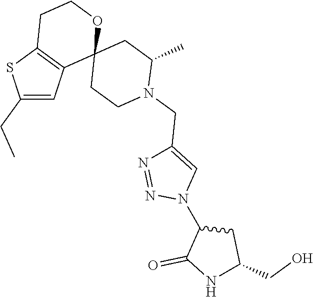 [SMILES: CCc1cc2c(s1)CCO[C@@]21CCN(Cc2cn(C3C[C@H](CO)NC3=O)nn2)[C@@H](C)C1] | 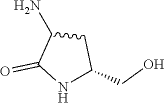 | Com- pound 15 | LCMS m/z [M + H]+ 446.2 |
| 197 | 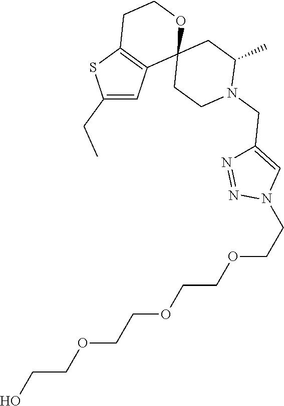 [SMILES: CCc1cc2c(s1)CCO[C@@]21CCN(Cc2cn(CCOCCOCCOCCO)nn2)[C@@H](C)C1] | 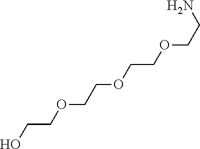 [SMILES: NCCOCCOCCOCCO] | Com- pound 15 | LCMS m/z [M + H]+ 509.27 |

[symbol]

indicates data missing or illegible when filed

### Compound 198

### (2′S,4R)-1′-[(2-chloropyrimidin-5-yl)methyl]-2-ethyl-2′-methyl-spiro[6,7-dihydrothieno[3,2-c]pyran-4,4′-piperidine](198)

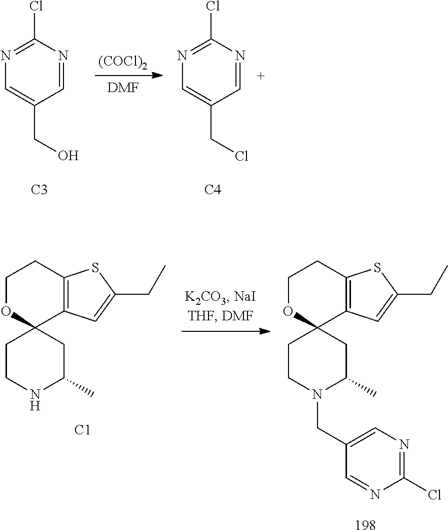

### Step 1. 2-chloro-5-(chloromethyl)pyrimidine (C4)

A bottom flask was charged with (2-chloropyrimidin-5-yl)methanol (1 g, 6.92 mmol) and thionyl chloride (12.5 mL, 171 mmol). To the suspension was added DMF (32.5 μL, 0.42 mmol) and the resulting mixture was heated at reflux for 5 h. Upon heating, the mixture dissolved to form a clear yellow homogenous solution. Upon complete conversion, the reaction was cooled to ambient temperature, and then concentrated in vacuo. The crude was azeotroped with dichloroethane (×2) and dried to constant mass under high vacuum to afford C4 (1.08 g, 92%). ¹H NMR (300 MHz, Chloroform-d) δ 8.67 (s, 2H), 4.57 (d, J=0.6 Hz, 2H). LCMS m/z 331.25 [M+H]⁺.

### Step 2. (2′S,4R)-1′-[(2-chloropyrimidin-5-yl)methyl]-2-ethyl-2′-methyl-spiro[6,7-dihydrothieno [3,2-c]pyran-4,4′-piperidine](198)

A solution of (2′S,4R)-2-ethyl-2′-methyl-spiro[6,7-dihydrothieno[3,2-c]pyran-4,4′-piperidine] (Triflate salt) (2 g, 4.99 mmol), 2-chloro-5-(chloromethyl)pyrimidine (3 g, 6.07 mmol), K₂CO₃ (2.2 g, 15.92 mmol) and NaI (750 mg, 5.00 mmol) in THF (18 mL) and DMF (2 mL) was heated at 40° C. overnight. The mixture was diluted with H₂O (30 mL) and extracted with EtOAc (50 mL×2). The combined organic extracts were washed with 2N Na₂S₂O₃ (20 mL) and brine (20 mL), dried over Na₂SO₄, filtered and concentrated in vacuo. The crude was purified by silica gel chromatography eluting with 0-33% EtOAc/Hexanes to afford the 198 (1.34 g, 70%). ¹H NMR (300 MHz, Chloroform-d) δ 8.62 (s, 2H), 6.48 (d, J=1.1 Hz, 1H), 4.12 (d, J=14.2 Hz, 1H), 4.01-3.67 (m, 2H), 3.19 (d, J=14.2 Hz, 1H), 2.89-2.61 (m, 5H), 2.57-2.30 (m, 2H), 2.00-1.53 (m, 4H), 1.29 (t, J=7.5 Hz, 3H), 1.17 (d, J=6.2 Hz, 3H). LCMS m/z 378.07 [M+H]⁺.

### Compound 199

### (2′S,4R)-1′-[(6-chloro-3-pyridyl)methyl]-2-ethyl-2′-methyl-spiro[6,7-dihydrothieno[3,2-c]pyran-4,4′-piperidine](199)

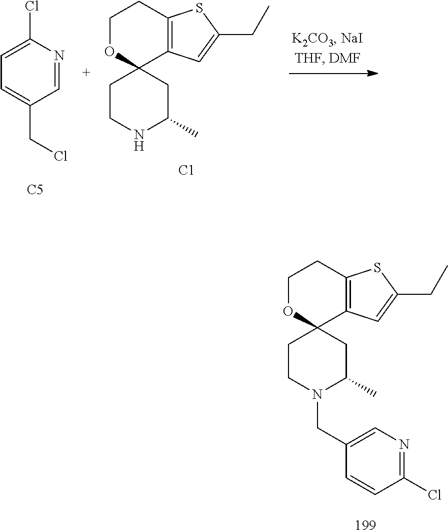

### Preparation of (2′S,4R)-1′-[(6-chloro-3-pyridyl)methyl]-2-ethyl-2′-methyl-spiro[6,7-dihydrothieno[3,2-c]pyran-4,4′-piperidine](199)

A mixture of (2′S,4R)-2-ethyl-2′-methyl-spiro[6,7-dihydrothieno[3,2-c]pyran-4,4′-piperidine] (Triflate salt) S1 (2 g, 4.99 mmol), 2-chloro-5-(chloromethyl)pyridine (900 mg, 5.56 mmol), K₂CO₃ (2.2 g, 15.92 mmol) and NaI (750 mg, 5.00 mmol) in THF (18 mL) and DMF (2 mL) was heated at 30° C. overnight. The mixture was diluted with H₂O (30 mL) and extracted with EtOAc (50 mL×2). The combined organic extracts were washed with brine (20 mL), dried over Na₂SO₄, filtered and concentrated in vacuo. The crude was purified by silica gel chromatography eluting with 0-33% EtOAc/Hexanes to afford the 199 (2.00 g, 92%). ¹H NMR (300 MHz, Chloroform-d) 8.30 (d, J=2.4 Hz, 1H), 7.65 (dd, J=8.2, 2.4 Hz, 1H), 7.23 (d, J=8.2 Hz, 1H), 6.45 (d, J=1.1 Hz, 1H), 4.06 (d, J=13.8 Hz, 1H), 3.84 (tdd, J=11.3, 8.8, 4.9 Hz, 2H), 3.11 (d, J=13.8 Hz, 1H), 2.87-2.55 (m, 5H), 2.57-2.24 (m, 2H), 1.96-1.58 (m, 4H), 1.23 (t, J=7.5 Hz, 3H), 1.12 (d, J=6.1 Hz, 3H). LCMS m/z 377.13 [M+H]⁺.

### Compound 200

### [(1R,4S)-4-[[5-[[(2′S,4R)-2-ethyl-2′-methyl-spiro[6,7-dihydrothieno[3,2-c]pyran-4,4′-piperidine]-1′-yl]methyl]pyrimidin-2-yl]amino]cyclopent-2-en-1-yl]methanol (200)

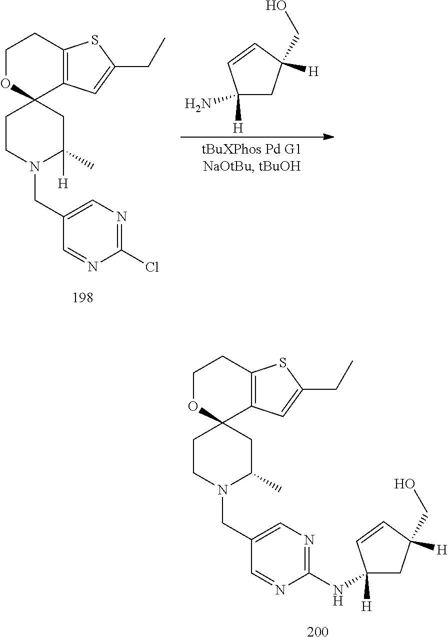

### Preparation of [(1R,4S)-4-[[5-[[(2′S,4R)-2-ethyl-2′-methyl-spiro[6,7-dihydrothieno[3,2-c]pyran-4,4′-piperidine]-1′-yl]methyl]pyrimidin-2-yl]amino]cyclopent-2-en-1-yl]methanol (200)

A mixture of (2′S,4R)-1′-[(2-chloropyrimidin-5-yl)methyl]-2-ethyl-2′-methyl-spiro[6,7-dihydrothieno [3,2-c]pyran-4,4′-piperidine] (14.5 mg, 0.037 mmol), ((1R,4S)-4-aminocyclopent-2-en-1-yl)methanol (5.89 mg, 0.052 mmol), tBuXPhos Pd G1 (2.41 mg, 0.0037 mmol) in tBuOH (560 μL) was degassed with N₂, and then a 2M solution of NaOtBu in tBuOH (48 μL) was added. The resulting mixture was heated at 85° C. for 3 h. After cooling down to ambient temperature, the reaction was quenched with MeOH. The solvent was removed in vacuo and the crude was purified by reversed phase chromatography (C18 column; Gradient: MeCN in H₂O with 0.1% TFA) to afford the 200 (2.2 mg, 8.3%). LCMS m/z 455.58 [M+H]⁺.

### Compound 201

### 2-[[5-[[(2′S,4R)-2-ethyl-2′-methyl-spiro[6,7-dihydrothieno[3,2-c]pyran-4,4′-piperidine]-1′-yl]methyl]pyrimidin-2-yl]amino]-2-methyl-propan-1-ol (201)

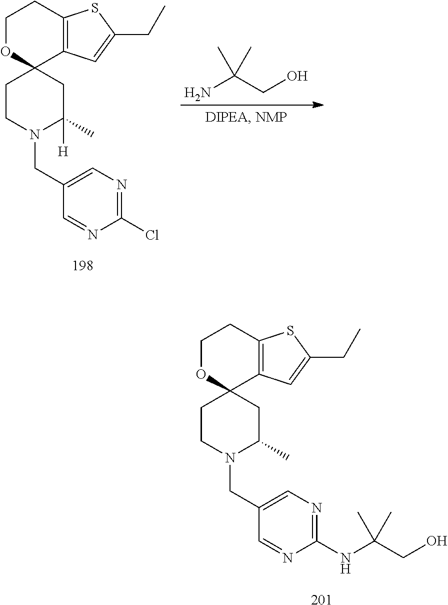

### Preparation of 2-[[5-[[(2′S,4R)-2-ethyl-2′-methyl-spiro[6,7-dihydrothieno[3,2-c]pyran-4,4′-piperidine]-1′-yl]methyl]pyrimidin-2-yl]amino]-2-methyl-propan-1-ol (201)

To a mixture of 2-amino-2-methyl-propan-1-ol (50 mg, 0.56 mmol) and (2′S,4R)-1′-[(2-chloropyrimidin-5-yl)methyl]-2-ethyl-2′-methyl-spiro[6,7-dihydrothieno[3,2-c]pyran-4,4′-piperidine]198 (30 mg, 0.069 mmol) in NMP (2 mL) was added DIPEA (51 mg, 0.39 mmol). The reaction was heated at 230° C. in a microwave for 6 h. After cooling down to rt, the reaction was diluted with DCM, washed with H₂O (×3). The organic layer was separated and concentrated in vacuo. The crude was purified by reversed phase chromatography (C18 column; Gradient: MeCN in H₂O with 0.1% TFA) to afford the 201 as a TFA salt (3.6 mg, 9%). ¹H NMR (300 MHz, Methanol-d4) δ 8.24 (s, 2H), 6.49 (d, J=1.0 Hz, 1H), 3.97 (d, J=13.6 Hz, 1H), 3.92-3.81 (m, 2H), 3.67 (s, 2H), 3.21 (d, J=13.6 Hz, 1H), 2.78-2.61 (m, 6H), 2.44 (td, J=10.9, 5.3 Hz, 1H), 1.87-1.66 (m, 4H), 1.38 (s, 6H), 1.27-1.20 (m, 6H). LCMS m/z 431.49 [M+H]⁺.

### Compound 202

### 5-[[(2′S)-2-ethyl-2′-methyl-spiro[6,7-dihydrothieno[3,2-c]pyran-4,4′-piperidine]-1′-yl]methyl]-N-methyl-pyridin-2-amine (202)

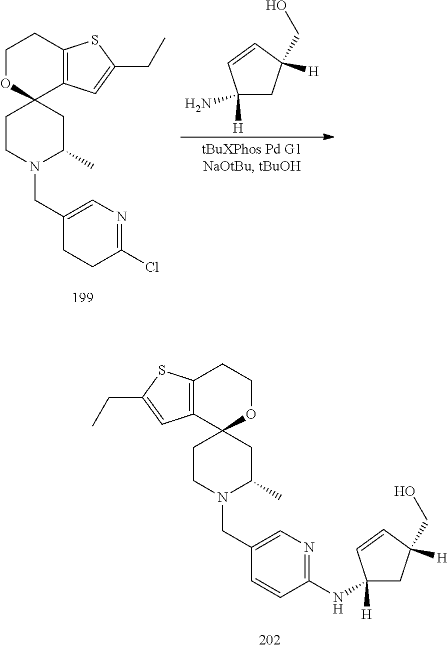

### Preparation of 5-[[(2′S)-2-ethyl-2′-methyl-spiro[6,7-dihydrothieno[3,2-c]pyran-4,4′-piperidine]-1′-yl]methyl]-N-methyl-pyridin-2-amine (202)

A mixture of (2′S)-1′-[(6-chloro-3-pyridyl)methyl]-2-ethyl-2′-methyl-spiro[6,7-dihydrothieno[3,2-c]pyran-4,4′-piperidine]199 (15 mg, 0.038 mmol), ((1R,4S)-4-aminocyclopent-2-en-1-yl)methanol (5.21 mg, 0.046 mmol), tBuXPhos Pd G1 (2.50 mg, 0.0038 mmol) in tBuOH (500 μL) was degassed with N₂, and then a 2M solution of NaOtBu in tBuOH (42 μL) was added. The resulting mixture was heated at 60° C. for 3 h. After cooling down to ambient temperature, the reaction was quenched with MeOH. The solvent was removed in vacuo and the crude was purified by reversed phase chromatography (C18 column; Gradient: MeCN in H₂O with 0.1% TFA) to afford 202 (11.9 mg, 49%). ¹H NMR (300 MHz, Chloroform-d) δ 8.29 (dd, J=18.9, 9.3 Hz, 1H), 7.99 (s, 1H), 7.03 (d, J=9.6 Hz, 1H), 6.49 (s, 1H), 6.12-6.02 (m, 1H), 5.95 (s, 1H), 5.91-5.81 (m, 1H), 4.84-4.62 (m, 2H), 4.45 (s, 1H), 4.02-3.49 (m, 6H), 3.26-2.88 (m, 3H), 2.77 (q, J=7.7 Hz, 4H), 2.58 (dt, J=14.1, 8.7 Hz, 1H), 2.36 (q, J=13.4, 12.3 Hz, 2H), 2.12-1.91 (m, 2H), 1.83 (dt, J=14.2, 3.2 Hz, 1H), 1.60 (dd, J=6.5, 3.6 Hz, 3H), 1.27 (t, J=7.5 Hz, 3H).

### Compounds 203-231

Compounds 203-231 (see Table 4) were prepared from 198 or 199 using the appropriate amines with general methods for 200-202. All amines were obtained from commercial sources, unless noted otherwise. Any modifications to methods are noted in Table 4 and accompanying footnotes.

TABLE 4
Structure and physicochemical data for compounds 203-231

| | | | | ¹H NMR; LCMS m/z |
|---|---|---|---|---|
| Compd | Structure | Amine | Method | [M + H]⁺ |
| 203 | 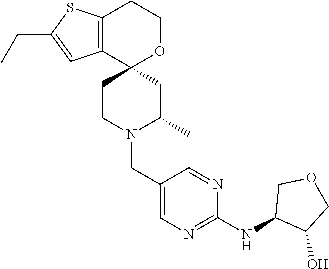 [SMILES: CCc1cc2c(s1)CCO[C@@]21CCN(Cc2cnc(N[C@H]3COC[C@@H]3O)nc2)[C@@H](C)C1] | 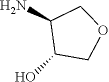 | Compound 200 | LCMS m/z [M + H]⁺ 445.35 |
| 204 | 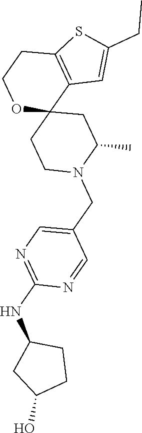 [SMILES: CCc1cc2c(s1)CCO[C@@]21CCN(Cc2cnc(N[C@H]3CC[C@H](O)C3)nc2)[C@@H](C)C1] | 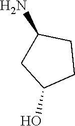 | Compound 200 | LCMS m/z [M + H]⁺ 443.33 |
| 205 | 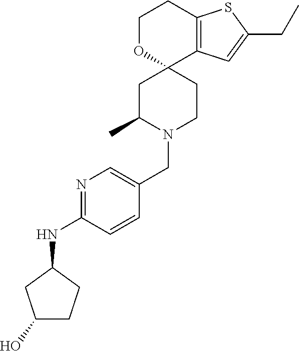 [SMILES: CCc1cc2c(s1)CCO[C@@]21CCN(Cc2ccc(N[C@H]3CC[C@H](O)C3)nc2)[C@@H](C)C1] | 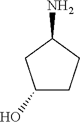 | Compound 202 | 1H NMR (300 MHz, Chloroform-d) δ 8.26 (d, J = 9.4 Hz, 1H), 7.88 (s, 1H), 6.96 (d, J = 9.5 Hz, 1H), 6.50 (s, 1H), 4.76 (d, J = 13.5 Hz, 1H), 4.54 (d, J = 5.7 Hz, 1H), 4.20 (s, 1H), 4.06-3.78 (m, 2H), 3.64-3.46 (m, 1H), 3.19 (d, J = 11.5 Hz, 1H), 2.98 (t, J = 12.4 Hz, 1H), 2.77 (q, J = 7.8 Hz, 5H), 2.40 (dd, J = 16.1, 9.5 Hz, 3H), 2.30-2.12 (m, 2H), 1.98 (dt, J = 19.6, 10.7 Hz, 3H), 1.83- 1.68 (m, 2H), 1.61 (d, J = 6.4 Hz, 4H), 1.27 (t, J = 7.5 Hz, 4H). LCMS m/z [M + H]⁺ 442.53 |
| 206 | 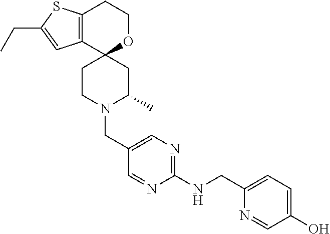 [SMILES: CCc1cc2c(s1)CCO[C@@]21CCN(Cc2cnc(NCc3ccc(O)cn3)nc2)[C@@H](C)C1] | 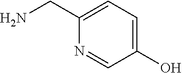 | Compound 200 | LCMS m/z [M + H]⁺ 466.1 |
| 207 | 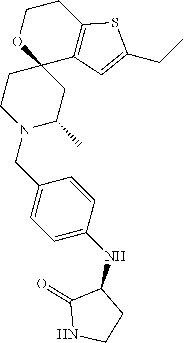 [SMILES: CCc1cc2c(s1)CCO[C@@]21CCN(Cc2ccc(N[C@H]3CCNC3=O)cc2)[C@@H](C)C1] | 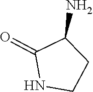 | Compound 201 | 1H NMR (300 MHz, Chloroform-d) δ 8.42 (s, 2H), 6.54 (s, 1H), 5.32 (d, J = 3.2 Hz, 1H), 4.54 (s, 2H), 3.86 (d, J = 16.6 Hz, 3H), 3.51 (d, J = 3.3 Hz, 5H), 3.44-3.13 (m, 4H), 3.13-2.60 (m, 6H), 2.60-2.19 (m, 2H), 2.03 (d, J = 18.1 Hz, 3H), 1.27 (dd, J = 8.7, 6.2 Hz, 3H). LCMS m/z [M + H]⁺ 442.2 |
| 208 | 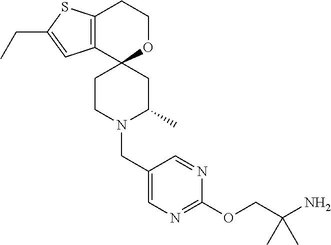 [SMILES: CCc1cc2c(s1)CCO[C@@]21CCN(Cc2cnc(OCC(C)(C)N)nc2)[C@@H](C)C1] | 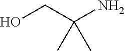 | Compound 200 | 1H NMR (300 MHz, Methanol-d4) δ 8.56 (s, 2H), 6.48 (d, J = 1.1 Hz, 1H), 4.21 (s, 2H), 4.10 (d, J = 13.8 Hz, 1H), 3.87 (td, J = 5.7, 4.3 Hz, 2H), 2.79- 2.56 (m, 7H), 2.45 (td, J = 11.4, 4.2 Hz, 1H), 1.87-1.67 (m, 4H), 1.27 (d, J = 4.1 Hz, 6H), 1.25-1.20 (m, 6H). LCMS m/z [M + H]⁺ 431.4 |
| 209 | 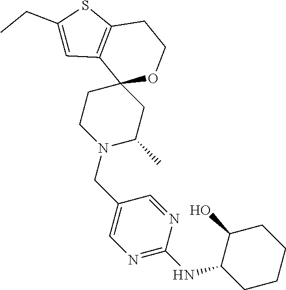 [SMILES: CCc1cc2c(s1)CCO[C@@]21CCN(Cc2cnc(N[C@H]3CCCC[C@@H]3O)nc2)[C@@H](C)C1] | 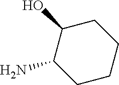 | Compound 200 | LCMS m/z [M + H]⁺ 457.24 |
| 210 | 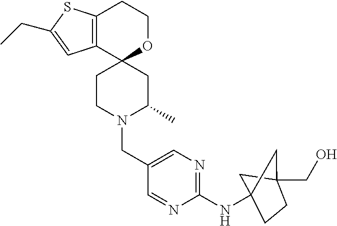 [SMILES: CCc1cc2c(s1)CCO[C@@]21CCN(Cc2cnc(NC34CCC(CO)(C3)C4)nc2)[C@@H](C)C1] | 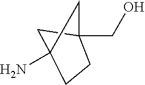 | Compound 200 | LCMS m/z [M + H]⁺ 469.26 |
| 211 | 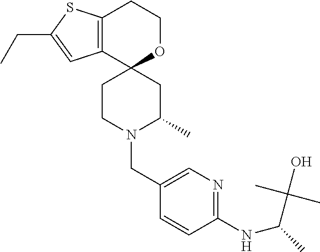 [SMILES: CCc1cc2c(s1)CCO[C@@]21CCN(Cc2ccc(N[C@@H](C)C(C)(C)O)nc2)[C@@H](C)C1] | 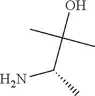 | Compound 202 | 1H NMR (300 MHz, Chloroform-d) δ 8.13 (s, 2H), 7.15 (d, J = 9.6 Hz, 1H), 6.42 (s, 1H), 4.74 (d, J = 13.4 Hz, 1H), 4.01-3.80 (m, 2H), 3.78-3.57 (m, 3H), 3.19 (dd, J = 35.8, 12.3 Hz, 2H), 2.92-2.67 (m, J = 6.1 Hz, 4H), 2.41-1.96 (m, 4H), 1.61 (s, 3H), 1.41-1.18 (m, 12H). LCMS m/z [M + H]⁺ 444.25 |
| 212 | 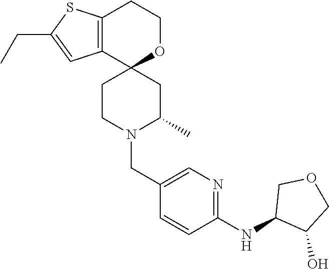 [SMILES: CCc1cc2c(s1)CCO[C@@]21CCN(Cc2ccc(N[C@H]3COC[C@@H]3O)nc2)[C@@H](C)C1] | 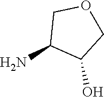 | Compound 202 | 1H NMR (300 MHz, Methanol-d4) δ 8.15 (s, 1H), 7.86 (d, J = 9.2 Hz, 1H), 6.98 (d, J = 9.2 Hz, 1H), 6.49 (s, 1H), 4.75 (d, J = 13.7 Hz, 1H), 4.35-3.60 (m, 10H), 2.76 (d, J = 7.1 Hz, 5H), 2.19- 1.99 (m, 4H), 1.56 (d, J = 6.4 Hz, 3H), 1.24 (t, J = 7.5 Hz, 4H). LCMS m/z [M + H]⁺ 444.21 |
| 213 | 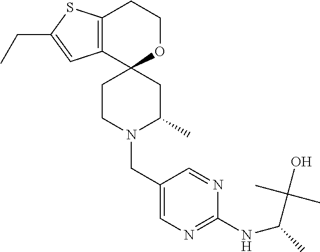 [SMILES: CCc1cc2c(s1)CCO[C@@]21CCN(Cc2cnc(N[C@@H](C)C(C)(C)O)nc2)[C@@H](C)C1] | 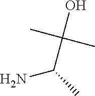 | Compound 200 | LCMS m/z [M + H]⁺ 445.29 |
| 214 | 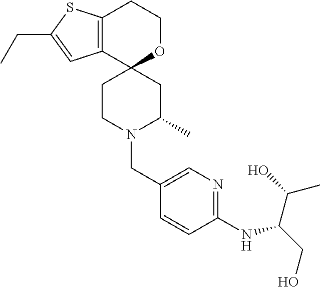 [SMILES: CCc1cc2c(s1)CCO[C@@]21CCN(Cc2ccc(N[C@@H](CO)[C@@H](C)O)nc2)[C@@H](C)C1] | 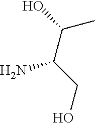 | Compound 202 | 1H NMR (300 MHz, Methanol-d4) δ 8.14- 7.91 (m, 2H), 7.20 (d, J = 9.3 Hz, 1H), 6.50 (s, 1H), 5.50 (s, 1H), 4.77 (d, J = 13.6 Hz, 1H), 4.11-3.56 (m, 7H), 2.76 (d, J = 7.1 Hz, 4H), 2.21-1.95 (m, 4H), 1.56 (d, J = 6.4 Hz, 3H), 1.35- 1.18 (m, 7H), 0.88 (s, 1H). LCMS m/z [M + H]⁺ 446.27 |
| 215 | 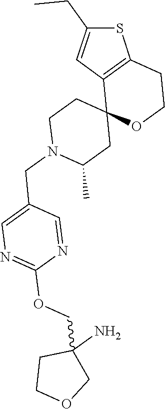 [SMILES: CCc1cc2c(s1)CCO[C@@]21CCN(Cc2cnc(OCC3(N)CCOC3)nc2)[C@@H](C)C1] | 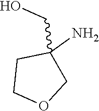 | Compound 200 | LCMS m/z [M + H]⁺ 459.58 |
| 216 | 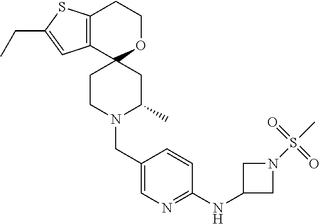 | 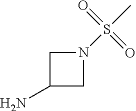 | Compound 202 | 1H NMR (300 MHz, Methanol-d4) δ 8.26- 8.15 (m, 2H), 7.18 (dd, J = 9.1, 0.9 Hz, 1H), 6.58 (d, J = 1.1 Hz, 1H), 4.83 (d, J = 6.6 Hz, 1H), 4.67 (tt, J = 7.2, 5.5 Hz, 1H), 4.39-4.30 (m, 2H), 4.07 (d, J = 13.6 Hz, 1H), 4.03-3.90 (m, 4H), 3.71 (ddd, J = 12.0, 6.4, 3.0 Hz, 1H), 3.27-3.16 (m, 2H), |
| | | | | 3.01 (s, 3H), 2.80- |
| | | | | 2.71 (m, 4H), 2.39- |
| | | | | 2.23 (m, 2H), 2.17- |
| | | | | 1.96 (m, 2H), 1.61 (d, |
| | | | | J = 6.4 Hz, 3H), 1.24 |
| | | | | (t, J = 7.5 Hz, 3H). |
| | | | | LCMS m/z [M + H]⁺ |
| | | | | 491.19 |
| 217 | 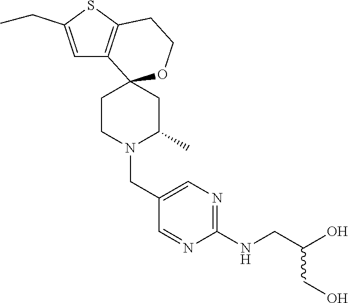 [SMILES: CCc1cc2c(s1)CCO[C@@]21CCN(Cc2cnc(NCC(O)CO)nc2)[C@@H](C)C1] | 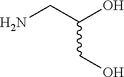 | Compound 200 | LCMS m/z [M + H]⁺ 433.24 |
| 218 | 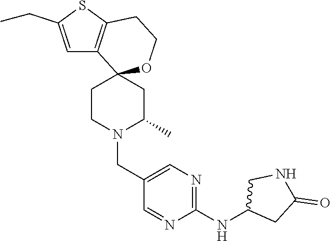 [SMILES: CCc1cc2c(s1)CCO[C@@]21CCN(Cc2cnc(NC3CNC(=O)C3)nc2)[C@@H](C)C1] | 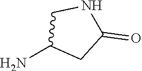 | Compound 200 | LCMS m/z [M + H]⁺ 442.32 |
| 219 | 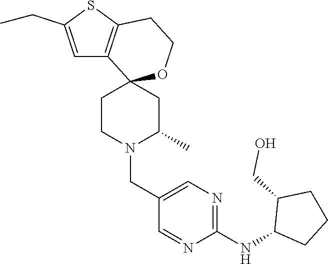 [SMILES: CCc1cc2c(s1)CCO[C@@]21CCN(Cc2cnc(N[C@H]3CCC[C@H]3CO)nc2)[C@@H](C)C1] | 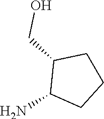 | Compound 200 | LCMS m/z [M + H]⁺ 457.24 |
| 220 | 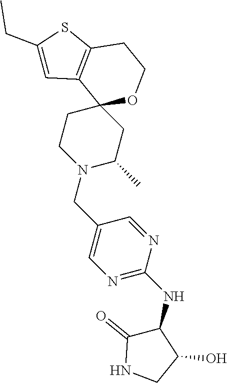 [SMILES: CCc1cc2c(s1)CCO[C@@]21CCN(Cc2cnc(N[C@@H]3C(=O)NC[C@H]3O)nc2)[C@@H](C)C1] | 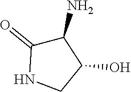 | Compound 200 | 1H NMR (400 MHz, Methanol-d4) δ 8.90 (d, J = 36.8 Hz, 2H), 6.59 (t, J = 1.0 Hz, 1H), 4.88 (d, J = 13.9 Hz, 1H), 4.81-4.52 (m, 2H), 4.23-3.61 (m, 6H), 3.33 (d, J = 2.6 Hz, 1H), 3.21 (dd, J = 9.9, 7.6 Hz, 1H), 2.86-2.71 (m, 4H), 2.38-1.97 (m, 4H), 1.62 (d, J = 6.4 Hz, 3H), 1.24 (t, J = 7.5 Hz, 3H). [3] LCMS m/z [M + H]⁺ 458.27 [3] |
| 221 | 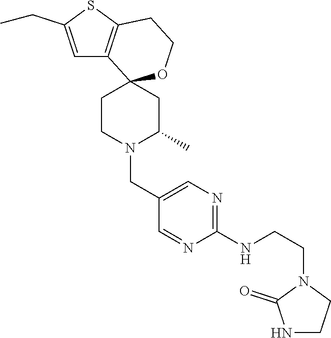 [SMILES: CCc1cc2c(s1)CCO[C@@]21CCN(Cc2cnc(NCCN3CCNC3=O)nc2)[C@@H](C)C1] | 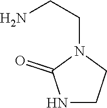 | Compound 200 | LCMS m/z [M + H]⁺ 471.09 |
| 222 | 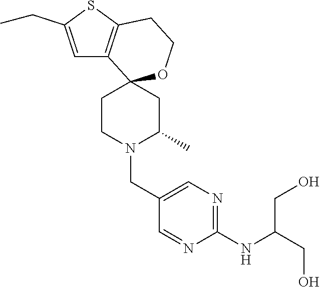 [SMILES: CCc1cc2c(s1)CCO[C@@]21CCN(Cc2cnc(NC(CO)CO)nc2)[C@@H](C)C1] | 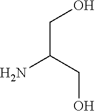 | Compound 200 | LCMS m/z [M + H]⁺ 433.24 |
| 223 | 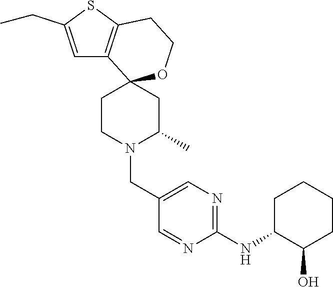 [SMILES: CCc1cc2c(s1)CCO[C@@]21CCN(Cc2cnc(N[C@@H]3CCCC[C@H]3O)nc2)[C@@H](C)C1] | 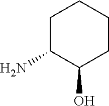 | Compound 200 | LCMS m/z [M + H]⁺ 457.28 |
| 224 | 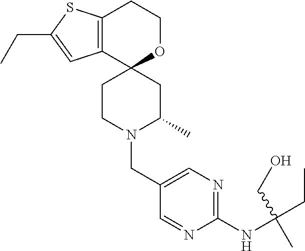 [SMILES: CCc1cc2c(s1)CCO[C@@]21CCN(Cc2cnc(NC(C)(CC)CO)nc2)[C@@H](C)C1] | 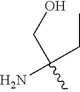 | Compound 201 | 1H NMR (300 MHz, Methanol-d4) δ 8.42 (s, 2H), 6.48 (s, 1H), 4.66 (d, J = 13.8 Hz, 1H), 4.11-3.52 (m, 6H), 2.76 (q, J = 7.3, 6.6 Hz, 5H), 2.22- 1.90 (m, 6H), 1.74 (dtd, J = 14.9, 7.7, 5.4 Hz, 1H), 1.56 (d, J = 6.4 Hz, 3H), 1.33 (s, 3H), 1.24 (t, J = 7.5 Hz, 3H), 1.08-0.75 (m, 3H). LCMS m/z [M + H]⁺ 445.26 |
| 225 | 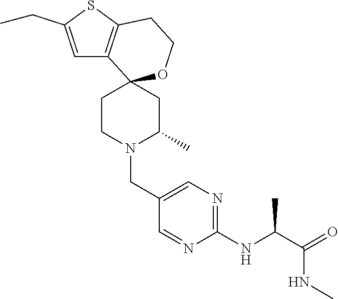 [SMILES: CCc1cc2c(s1)CCO[C@@]21CCN(Cc2cnc(N[C@@H](C)C(=O)NC)nc2)[C@@H](C)C1] | 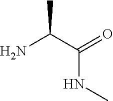 | Compound 201 | LCMS m/z [M + H]⁺ 444.21 |
| 226 | 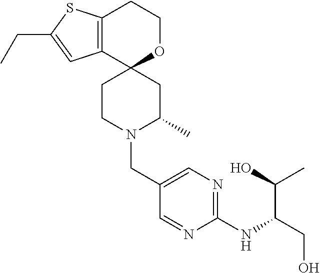 [SMILES: CCc1cc2c(s1)CCO[C@@]21CCN(Cc2cnc(N[C@@H](CO)[C@H](C)O)nc2)[C@@H](C)C1] | 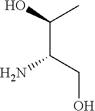 | Compound 201 | 1H NMR (300 MHz, Methanol-d4) δ 8.42 (d, J = 3.0 Hz, 2H), 6.47 (s, 1H), 4.67 (d, J = 13.9 Hz, 1H), 4.45- 3.49 (m, 7H), 2.78 (dt, J = 14.2, 6.5 Hz, 5H), 2.65 (s, 1H), 2.46- 1.88 (m, 4H), 1.56 (d, J = 6.4 Hz, 3H), 1.44- 0.98 (m, 6H). LCMS m/z [M + H]⁺ 447.24 |
| 227 | 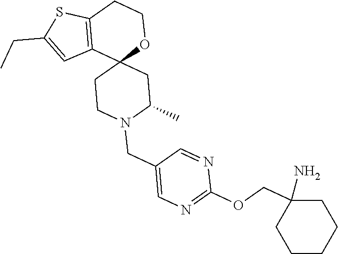 [SMILES: CCc1cc2c(s1)CCO[C@@]21CCN(Cc2cnc(OCC3(N)CCCCC3)nc2)[C@@H](C)C1] | 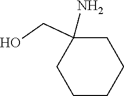 | Compound 200 | LCMS m/z [M + H]⁺ 471.09 |
| 228 | 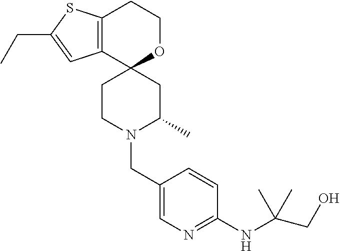 [SMILES: CCc1cc2c(s1)CCO[C@@]21CCN(Cc2ccc(NC(C)(C)CO)nc2)[C@@H](C)C1] | 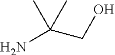 | Compound 202 | 1H NMR (300 MHz, Methanol-d4) δ 8.33 (d, J = 2.4 Hz, 1H), 7.93 (dd, J = 8.7, 2.5 Hz, 1H), 7.05 (d, J = 8.6 Hz, 1H), 6.48 (s, 1H), 4.80 (d, J = 13.6 Hz, 1H), 4.38 (s, 2H), 4.15 (d, J = 13.5 Hz, 1H), 3.92 (h, J = 5.9 Hz, 2H), 3.76-3.56 (m, 1H), 3.21 (td, J = 11.8, 7.6 Hz, 2H), 2.82- 2.68 (m, 4H), 2.21- 1.97 (m, 4H), 1.59 (d, J = 6.5 Hz, 3H), 1.46 (s, |
| | | | | 6H), 1.25 (q, J = 7.5, |
| | | | | 6.9 Hz, 3H). LCMS |
| | | | | m/z [M + H]⁺ 430.6 |
| 229 | 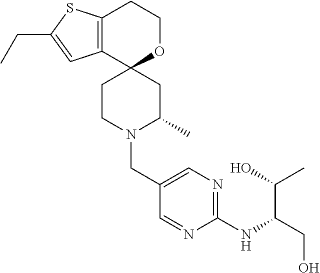 [SMILES: CCc1cc2c(s1)CCO[C@@]21CCN(Cc2cnc(N[C@@H](CO)[C@@H](C)O)nc2)[C@@H](C)C1] | 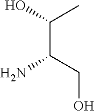 | Compound 201 | 1H NMR (300 MHz, Methanol-d4) δ 8.45 (s, 2H), 6.48 (s, 1H), 4.68 (d, J = 13.8 Hz, 1H), 4.18-3.86 (m, 5H), 3.86-3.42 (m, 4H), 2.91-2.59 (m, 5H), 2.34-1.91 (m, 5H), 1.56 (d, J = 6.4 Hz, 3H), 1.37-1.13 (m, 7H). LCMS m/z [M + H]⁺ 447.24 |
| 230 | 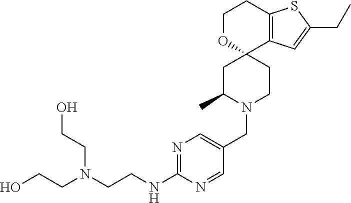 [SMILES: CCc1cc2c(s1)CCO[C@@]21CCN(Cc2cnc(NCCN(CCO)CCO)nc2)[C@@H](C)C1] | 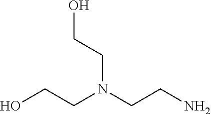 | Compound 200 | LCMS m/z [M + H]⁺ 490.31 |
| 231 | 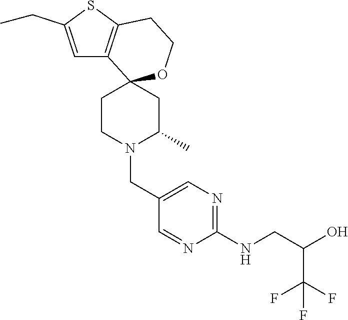 [SMILES: CCc1cc2c(s1)CCO[C@@]21CCN(Cc2cnc(NCC(O)C(F)(F)F)nc2)[C@@H](C)C1] | 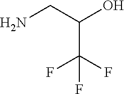 | Compound 200 | LCMS m/z [M + H]⁺ 471.19 |

### Compound 232

### 4-[(2′S,4R)-2-ethyl-2′-methyl-spiro[6,7-dihydrothieno[3,2-c]pyran-4,4′-piperidine]-1′-yl]tetrahydrofuran-3-ol (232) and 4-[(2′S,4R)-2-ethyl-2′-methyl-spiro[6,7-dihydrothieno[3,2-c]pyran-4,4′-piperidine]-1′-yl]tetrahydrofuran-3-ol (233)

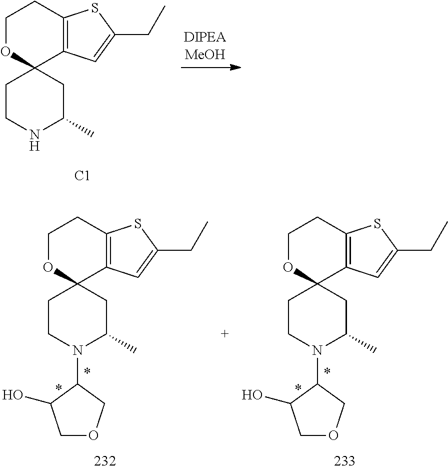

### Preparation of 4-[(2′S,4R)-2-ethyl-2′-methyl-spiro[6,7-dihydrothieno[3,2-c]pyran-4,4′-piperidine]-1′-yl]tetrahydrofuran-3-ol (232) and 4-[(2′S,4R)-2-ethyl-2′-methyl-spiro[6,7-dihydrothieno[3,2-c]pyran-4,4′-piperidine]-1′-yl]tetrahydrofuran-3-ol (233)

To a solution of (2′S,4R)-2-ethyl-2′-methyl-spiro[6,7-dihydrothieno[3,2-c]pyran-4,4′-piperidine] (Triflate salt) S1 (36 mg, 0.088 mmol) in MeOH (1 mL) was added DIPEA (100 μL, 0.57 mmol) and 3,6-dioxabicyclo[3.1.0]hexane (43 mg, 0.50 mmol). The mixture was heated in a microwave at 150° C. for 6 h. After cooling down to ambient temperature, the volatile was removed in vacuo and the crude was purified by reversed phase chromatography (C18 column; Gradient: MeCN in H₂O with 0.2% formic acid) to afford 232 (11.5 mg, 38%) and the corresponding trans diastereomer 233 (10 mg, 33%), Compound 232: ¹H NMR (300 MHz, Chloroform-d) δ 9.16 (s, 1H), 6.51 (d, J=1.1 Hz, 1H), 4.91 (t, J=6.6 Hz, 1H), 4.36 (dd, J=10.2, 7.2 Hz, 1H), 4.21 (d, J=11.9 Hz, 1H), 4.12 (d, J=5.9 Hz, 1H), 4.05-3.79 (m, 2H), 3.60 (dd, J=10.2, 5.9 Hz, 1H), 3.51-3.31 (m, 1H), 2.92-2.72 (m, 4H), 2.59 (s, 4H), 2.44-2.24 (m, 1H), 2.21-1.99 (m, 2H), 1.46 (d, J=6.6 Hz, 3H), 1.29 (t, J=7.5 Hz, 3H). LCMS m/z 338.21 [M+H]⁺. Compound 233: ¹H NMR (300 MHz, Chloroform-d) δ 8.56 (s, 1H), 6.54 (d, J=1.0 Hz, 1H), 4.73 (t, J=6.3 Hz, 1H), 4.35-4.01 (m, 3H), 3.99-3.79 (m, 2H), 3.60 (dd, J=9.8, 5.9 Hz, 1H), 3.49 (d, J=10.3 Hz, 1H), 3.18 (d, J=10.9 Hz, 1H), 2.98 (t, J=12.4 Hz, 1H), 2.86-2.71 (m, 5H), 2.40-2.20 (m, 1H), 2.13 (d, J=12.0 Hz, 1H), 1.98 (d, J=14.4 Hz, 2H), 1.43 (d, J=6.4 Hz, 3H), 1.28 (t, J=7.6 Hz, 3H). LCMS m/z 338.21 [M+H]⁺.

### Compounds 234-263

Compounds 234-263 (see Table 5) were prepared from C₁ using the appropriate epoxides employing the epoxide opening method described for 232 and 233. All epoxides were obtained from commercial sources, unless noted otherwise. Any modifications to methods are noted in Table 5 and accompanying footnotes.

TABLE 5
Structure and physicochemical data for compounds 234-263

| Compd | Structure | Epoxide | ¹H NMR; LCMS m/z [M + H]⁺ |
|---|---|---|---|
| 234 | 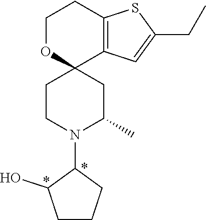 [SMILES: CCc1cc2c(s1)CCO[C@@]21CCN(C2CCCC2O)[C@@H](C)C1] | 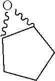 | LCMS m/z [M + H]⁺ 336.22 |
| 235 | 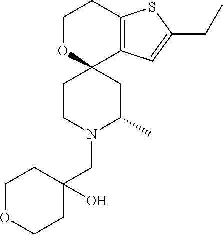 [SMILES: CCc1cc2c(s1)CCO[C@@]21CCN(CC2(O)CCOCC2)[C@@H](C)C1] | 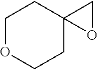 | 1H NMR (300 MHz, Chloroform- d) δ 8.45 (s, 1H), 6.55 (s, 1H), 4.01- 3.69 (m, 6H), 3.49-3.14 (m, 3H), 2.89-2.69 (m, 5H), 2.65 (s, 1H), 2.45 (s, 2H), 1.98 (q, J = 19.4, 17.1 Hz, 3H), 1.82-1.49 (m, 3H), 1.44 (d, J = 6.4 Hz, 3H), 1.29 (t, J = 7.5 Hz, 3H). LCMS m/z [M + H]⁺ 366.25 |
| 236 | 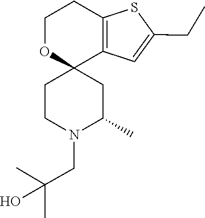 [SMILES: CCc1cc2c(s1)CCO[C@@]21CCN(CC(C)(C)O)[C@@H](C)C1] | 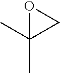 | 1H NMR (300 MHz, Chloroform- d) δ 8.67 (s, 1H), 6.55 (s, 1H), 4.01- 3.76 (m, 2H), 3.58 (s, 1H), 3.33- 3.01 (m, 3H), 2.77 (dd, J = 9.5, 6.0 Hz, 4H), 2.66-2.48 (m, 1H), 2.45- 2.12 (m, 2H), 2.04-1.78 (m, 2H), 1.48-1.10 (m, 12H). LCMS m/z [M + H]⁺ 324.26 |
| 237 | 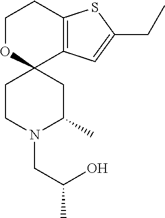 [SMILES: CCc1cc2c(s1)CCO[C@@]21CCN(C[C@@H](C)O)[C@@H](C)C1] | 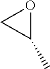 | 1H NMR (300 MHz, Chloroform- d) δ 8.57 (s, 1H), 6.53 (d, J = 1.0 Hz, 1H), 3.90 (tdd, J = 11.1, 8.6, 4.7 Hz, 3H), 3.51-3.36 (m, 1H), 3.33-3.13 (m, 2H), 2.92-2.71 (m, 5H), 2.67 (d, J = 15.6 Hz, 1H), 2.53-2.24 (m, 2H), 2.12-1.79 (m, 2H), 1.43 (d, J = 6.4 Hz, 3H), 1.33-1.16 (m, 6H). LCMS m/z [M + H]⁺ 310.22 |
| 238 | 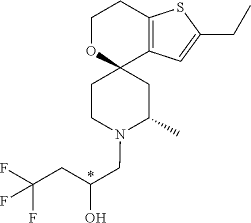 [SMILES: CCc1cc2c(s1)CCO[C@@]21CCN(CC(O)CC(F)(F)F)[C@@H](C)C1] | 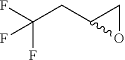 | LCMS m/z [M + H]⁺ 378.17 |
| 239 | 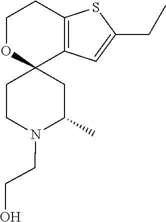 [SMILES: CCc1cc2c(s1)CCO[C@@]21CCN(CCO)[C@@H](C)C1] | 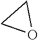 | 1H NMR (300 MHz, Chloroform- d) δ 8.25 (s, 1H), 6.58 (s, 1H), 4.06 (td, J = 4.4, 3.6, 1.5 Hz, 2H), 4.02- 3.84 (m, 3H), 3.69 (d, J = 3.6 Hz, 1H), 3.60 (s, 1H), 3.37 (s, 1H), 3.05 (d, J = 13.9 Hz, 1H), 2.92- 2.70 (m, 4H), 2.62-2.20 (m, 2H), 2.14-1.93 (m, 2H), 1.48 (d, J = 6.4 Hz, 3H), 1.28 (t, J = 7.5 Hz, 3H). LCMS m/z [M + H]⁺ 296.18 |
| 240 | 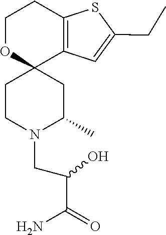 [SMILES: CCc1cc2c(s1)CCO[C@@]21CCN(CC(O)C(N)=O)[C@@H](C)C1] | 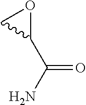 | 1H NMR (300 MHz, Chloroform- d) δ 6.52 (s, 1H), 5.53 (s, 1H), 4.70 (d, J = 9.7 Hz, 1H), 4.09-3.78 (m, 3H), 3.67 (d, J = 11.9 Hz, 1H), 3.50 (s, 1H), 3.30 (t, J = 12.9 Hz, 1H), 3.01-2.89 (m, 1H), 2.78 (t, J = 7.0 Hz, 4H), 2.36 (d, J = 13.2 Hz, 2H), 2.00 (d, J = 12.2 Hz, 2H), 1.48 (d, J = 6.4 Hz, 3H), 1.29 (t, J = 7.5 Hz, 3H). LCMS m/z [M + H]⁺ 339.21 |
| 241 | 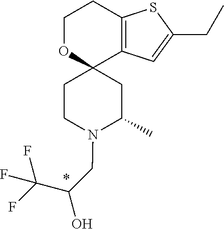 | 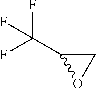 | LCMS m/z [M + H]⁺ 364.18 |
| 242 | 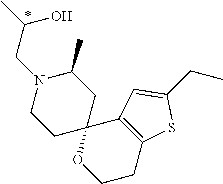 [SMILES: CCc1cc2c(s1)CCO[C@@]21CCN(CC(C)O)[C@@H](C)C1] | 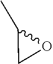 | 1H NMR (400 MHz, DMSO-d6): δ 6.59 (s, 1H), 4.07 (bs, 1H), 3.85- 3.71 (m, 3H), 2.74-2.57 (m, 8H), 2.07-1.52 (m, 5H), 1.23-1.17 (t, J = 7.6 Hz, 3H), 1.04-1.03 (d, J = 5.2 Hz, 3H), 0.98 (s, 3H). LCMS m/z [M + H]⁺ 310.2 |
| 243 | 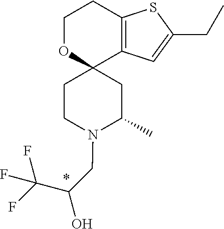 | 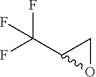 | LCMS m/z [M + H]⁺ 364.18 |
| 244 | 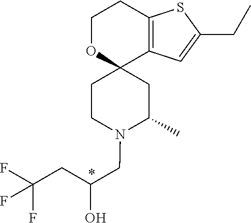 [SMILES: CCc1cc2c(s1)CCO[C@@]21CCN(CC(O)CC(F)(F)F)[C@@H](C)C1] | 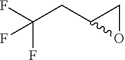 | LCMS m/z [M + H]⁺ 378.22 |
| 245 | 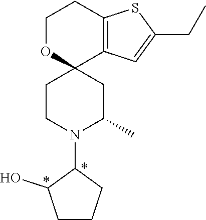 [SMILES: CCc1cc2c(s1)CCO[C@@]21CCN(C2CCCC2O)[C@@H](C)C1] | 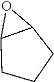 | LCMS m/z [M + H]⁺ 336.22 |
| 246 | 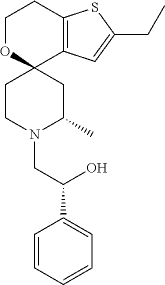 [SMILES: CCc1cc2c(s1)CCO[C@@]21CCN(C[C@H](O)c2ccccc2)[C@@H](C)C1] | 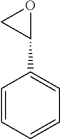 | LCMS m/z [M + H]⁺ 372.21 |
| 247 | 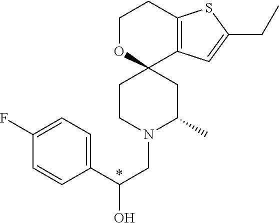 | 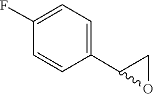 | 1H NMR (300 MHz, Chloroform- d) δ 8.56 (s, 1H), 6.54 (d, J = 1.0 Hz, 1H), 4.73 (t, J = 6.3 Hz, 1H), 4.35-4.01 (m, 3H), 3.99-3.79 (m, 2H), 3.60 (dd, J = 9.8, 5.9 Hz, 1H), 3.49 (d, J = 10.3 Hz, 1H), 3.18 (d, J = 10.9 Hz, 1H), 2.98 (t, J = 12.4 Hz, 1H), 2.86-2.71 (m, 5H), 2.40-2.20 (m, 1H), 2.13 (d, J = 12.0 Hz, 1H), 1.98 (d, J = 14.4 Hz, 2H), 1.43 (d, J = 6.4 Hz, 3H), 1.28 (t, J = 7.6 Hz, 3H). LCMS m/z [M + H]⁺ 338.21 |
| 248 | 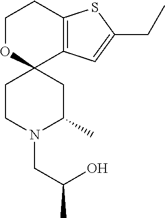 | 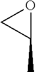 | LCMS m/z [M + H]⁺ 390.23 |
| 249 | 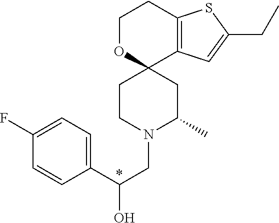 | 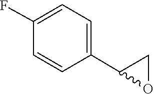 | LCMS m/z [M + H]⁺ 310.22 |
| 250 | 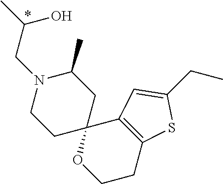 | 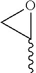 | LCMS m/z [M + H]⁺ 390.23 |
| 251 | 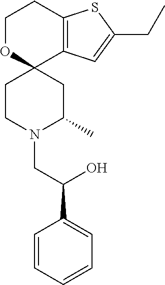 | 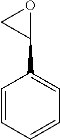 | 1H NMR (400 MHz, DMSO-d6): δ 6.59 (s, 1H), 4.33 (bs, 1H), 3.86- 3.72 (m, 3H), 2.73-2.64 (m, 6H), 2.51-2.49 (m, 2H), 2.10-1.51 (m, 5H), 1.21-1.17 (t, J = 7.6 Hz, 3H), 1.06-1.05 (d, J = 6.0 Hz, 3H), 0.98-0.97 (d, J = 5.2 Hz, 3H). LCMS m/z [M + H]⁺ 310.2 |
| 252 | 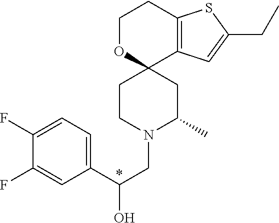 | 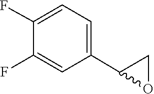 [SMILES: Fc1ccc(C2CO2)cc1F] | 1H NMR (300 MHz, Chloroform- d) δ 8.40 (s, 1H), 7.63-7.39 (m, 5H), 6.44 (s, 1H), 4.57 (dd, J = 7.9, 5.1 Hz, 1H), 4.44 (dd, J = 12.2, 7.9 Hz, 1H), 4.19 (dd, J = 12.1, 5.0 Hz, 1H), 4.01-3.72 (m, 2H), 3.50 (d, J = 14.6 Hz, 1H), 3.46-3.37 (m, 1H), 3.27 (d, J = 11.9 Hz, 1H), 2.84-2.66 (m, 5H), 2.35-2.15 (m, 1H), 1.94-1.72 (m, 2H), 1.49 (d, J = 6.4 Hz, 3H), 1.27 (t, J = 7.5 Hz, 3H). LCMS m/z [M + H]⁺ 372.25 |
| 253 | 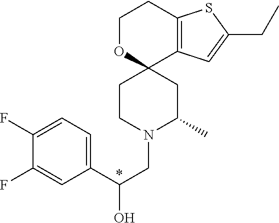 [SMILES: CCc1cc2c(s1)CCO[C@@]21CCN(CC(O)c2ccc(F)c(F)c2)[C@@H](C)C1] | 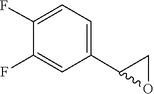 [SMILES: Fc1ccc(C2CO2)cc1F] | LCMS m/z [M + H]⁺ 408.2 |
| 254 | 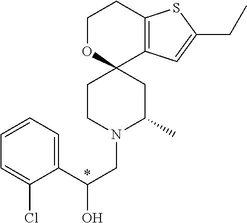 [SMILES: CCc1cc2c(s1)CCO[C@@]21CCN(CC(O)c2ccccc2Cl)[C@@H](C)C1] | 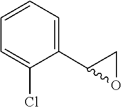 | LCMS m/z [M + H]⁺ 408.2 |
| 255 | 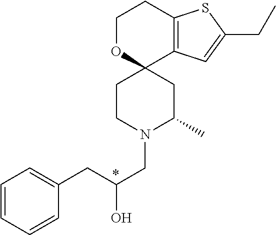 | 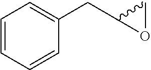 | LCMS m/z [M + H]⁺ 406.17 |
| 256 | 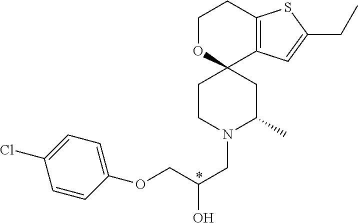 | 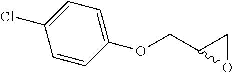 [SMILES: Clc1ccc(OCC2CO2)cc1] | LCMS m/z [M + H]⁺ 386.21 |
| 257 | 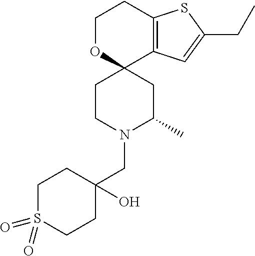 [SMILES: CCc1cc2c(s1)CCO[C@@]21CCN(CC2(O)CCS(=O)(=O)CC2)[C@@H](C)C1] | 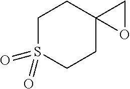 | LCMS m/z [M + H]⁺ 436.15 |
| 258 | 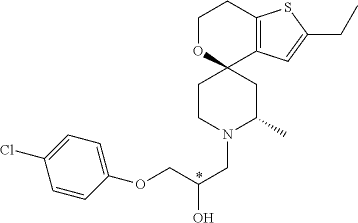 | 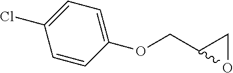 [SMILES: Clc1ccc(OCC2CO2)cc1] | 1H NMR (300 MHz, Chloroform- d) δ 6.49 (s, 1H), 3.91 (hept, J = 5.8 Hz, 2H), 3.63-3.34 (m, 2H), 3.04 (d, J = 37.0 Hz, 5H), 2.90 (d, J = 14.9 Hz, 1H), 2.78 (t, J = 6.1 Hz, 4H), 2.48 (d, J = 14.0 Hz, 1H), 2.16 (q, J = 15.5, 14.8 Hz, 6H), 1.87 (d, J = 17.8 Hz, 3H), 1.30 (t, J = 7.5 Hz, 3H), 1.25-1.10 (m, 3H). LCMS m/z [M + H]⁺ 414.16 |
| 259 | 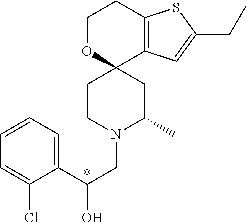 | 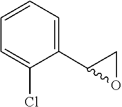 | LCMS m/z [M + H]⁺ 436.15 |
| 260 | 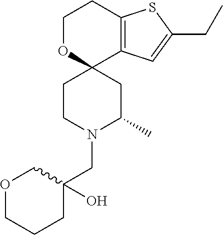 | 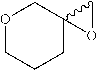 | LCMS m/z [M + H]⁺ 406.17 |
| 261 | 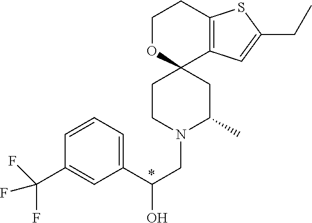 | 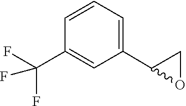 [SMILES: FC(F)(F)c1cccc(C2CO2)c1] | 1H NMR (300 MHz, Chloroform- d) δ 8.39 (s, 1H), 6.58 (d, J = 8.1 Hz, 1H), 4.08-3.74 (m, 6H), 3.66- 3.17 (m, 5H), 2.98 (d, J = 13.6 Hz, 1H), 2.79 (q, J = 7.6, 6.5 Hz, 4H), 2.46 (s, 2H), 2.15-1.72 (m, 4H), 1.69-1.52 (m, 1H), 1.46 (d, J = 6.4 Hz, 3H), 1.29 (td, J = 7.5, 1.0 Hz, 3H). LCMS m/z [M + H]⁺ 366.25 |
| 262 | 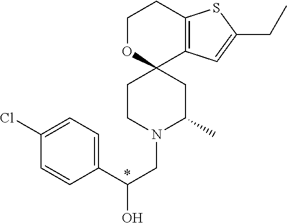 [SMILES: CCc1cc2c(s1)CCO[C@@]21CCN(CC(O)c2ccc(Cl)cc2)[C@@H](C)C1] | 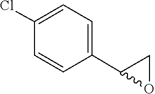 | LCMS m/z [M + H]⁺ 440.17 |
| 263 | 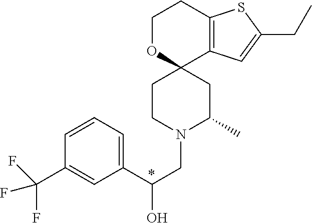 | 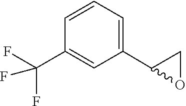 [SMILES: FC(F)(F)c1cccc(C2CO2)c1] | LCMS m/z [M + H]⁺ 406.17 |

### Preparation S9

### (2′S,4S)-2-ethyl-2′-methyl-spiro[6,7-dihydrothieno[3,2-c]pyran-4,4′-piperidine](S9)

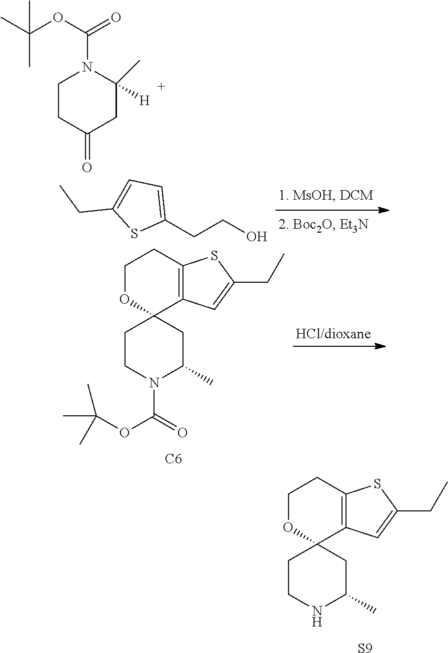

### Step 1: tert-butyl(2′S,4S)-2-ethyl-2′-methyl-spiro[6,7-dihydrothieno[3,2-c]pyran-4,4′-piperidine]-1′-carboxylate (C6)

A solution of 2-(5-ethyl-2-thienyl)ethanol (103 mg, 0.622 mmol), tert-butyl (2S)-2-methyl-4-oxo-piperidine-1-carboxylate (144 mg, 0.675 mmol) in DCM (1 mL) was cooled to −78° C. MsOH (0.1 mL, 1.54 mmol) was added dropwise and the resulting solution was stirred for 2 h. Triflic acid (0.1 mL, 1.13 mmol) was added and the reaction was stirred at −78° C. for 1 h. Water (15 mL) and DCM (15 mL) were added and the pH was adjusted to ˜10 using 2 N NaOH. The aqueous layer was extracted with DCM (3×15 mL) and filtered to give a yellow gel. The crude product was dissolved in DCM (1 mL) and Et₃N (0.2 mL, 1.43 mmol) was added followed by boc anhydride (0.2 mL, 0.870 mmol). The reaction mixture was stirred at rt for 3 h. The solution was diluted with DCM and washed with water (10 mL). The aqueous layer was extracted with DCM (2×10 mL), dried over Na₂SO₄ and filtered. Purification by silica gel chromatography (Gradient: 0-60% EtOAc in heptane) gave tert-butyl(2′S,4S)-2-ethyl-2′-methyl-spiro[6,7-dihydrothieno[3,2-c]pyran-4,4′-piperidine]-1′-carboxylate C6 (117 mg, 50%). 1H NMR (300 MHz, Methanol-d4) δ 6.46 (s, 1H), 4.43-4.29 (m, 1H), 3.97-3.83 (m, 3H), 2.84-2.61 (m, 4H), 1.94-1.76 (m, 4H), 1.48 (s, 9H), 1.33 (d, J=7.1 Hz, 3H), 1.24 (t, J=7.5 Hz, 3H). (1H under MeOH peak).

### Step 2: (2′S,4S)-2-ethyl-2′-methyl-spiro[6,7-dihydrothieno[3,2-c]pyran-4,4′-piperidine]S9

To a stirred solution of tert-butyl(2′S,4S)-2-ethyl-2′-methyl-spiro[6,7-dihydrothieno[3,2-c]pyran-4,4′-piperidine]-1′-carboxylate C6 (105 mg, 0.299 mmol) in DCM (1 mL) was added 4 M HCl in dioxane (0.16 mL of 4 M, 0.660 mmol) and the reaction was stirred at rt for 4 h. Concentrated and purified by Prep-HPLC (Mobile phase A: −0.1% FA (aq), Mobile phase B: -Acetonitrile) to yield (2′S,4S)-2-ethyl-2′-methyl-spiro[6,7-dihydrothieno[3,2-c]pyran-4,4′-piperidine]S9 (65 mg, 72%). ¹H NMR (300 MHz, Methanol-d4) δ 6.56 (d, J=1.6 Hz, 1H), 3.94 (t, J=5.4 Hz, 2H), 3.81-3.70 (m, 1H), 3.50 (td, J=12.4, 3.6 Hz, 1H), 3.19 (dt, J=13.0, 4.1 Hz, 1H), 2.82-2.70 (m, 4H), 2.15 (td, J=14.7, 14.2, 4.8 Hz, 2H), 2.02 (d, J=15.2 Hz, 2H), 1.55 (d, J=7.1 Hz, 3H), 1.25 (t, J=7.5 Hz, 3H).

### Preparation S10

### 2-(4-formylpyrazol-1-yl)-N-methyl-ethanesulfonamide (S10)

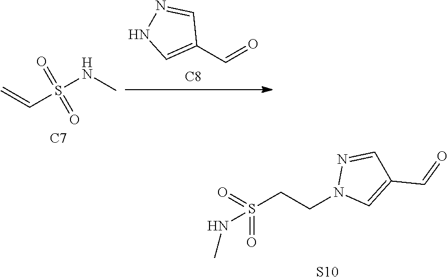

### Step 1: 2-(4-formylpyrazol-1-yl)-N-methyl-ethanesulfonamide (S10)

A solution of 1H-pyrazole-4-carbaldehyde C8 (250 mg, 2.60 mmol), N-methylethenesulfonamide C7 (350 mg, 2.88 mmol) and potassium carbonate (700 mg, 5.06 mmol) in 2-methyltetrahydrofuran (10 mL) was stirred at 60° C. After stirring for 4 h, the mixture was cooled to room temperature and the reaction was quenched with water (10 mL). The organic layer was diluted with EtOAc (40 mL). The layers were separated, and the aqueous layer was extracted with EtOAc (2×30 mL) followed by DCM (2×20 mL), dried over Na₂SO₄, filtered and concentrated. Purification by silica gel chromatography (Gradient: 0-10% Methanol in DCM) gave 2-(4-formylpyrazol-1-yl)-N-methyl-ethanesulfonamide S10 (565 mg, 54%). ¹H NMR (400 MHz, DMSO-d6) δ δ 9.80 (s, 1H), 8.53 (d, J=0.7 Hz, 1H), 8.03 (d, J=0.7 Hz, 1H), 7.15 (d, J=4.3 Hz, 1H), 4.53 (dd, J=7.4, 6.5 Hz, 2H), 3.60 (dd, J=7.4, 6.5 Hz, 2H), 2.56 (d, J=3.5 Hz, 3H). LCMS m/z 218.42 [M+H]⁺.

### Preparation S11

### 1-(2-methylsulfonylethyl)pyrazole-4-carbaldehyde (S11)

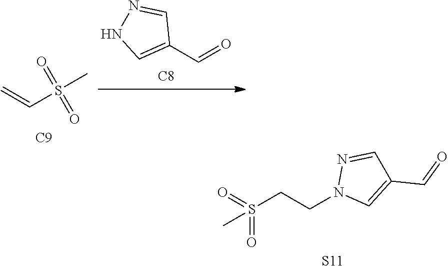

### Step 1: 1-(2-methylsulfonylethyl)pyrazole-4-carbaldehyde (S11)

A solution of 1H-pyrazole-4-carbaldehyde C8 (1 g, 10.4 mmol), 1-methylsulfonylethylene C9 (1.2 g, 11.4 mmol) and Potassium Carbonate (2.4 g, 18.1 mmol) in 2-methyltetrahydrofuran (20 mL) was stirred at 60° C. After stirring overnight, the mixture was cooled to room temperature and the reaction was quenched with water (10 mL). The organic layer was diluted with EtOAc (40 mL). The layers were separated, and the aqueous layer was extracted with EtOAc (2×30 mL) followed by DCM (2×20 mL), dried over Na₂SO₄, filtered and concentrated. Purification by silica gel chromatography (Gradient: 0-10% Methanol in DCM) gave 1-(2-methylsulfonylethyl)pyrazole-4-carbaldehyde S11 (1.3 g, 58%). 1H NMR (400 MHz, Chloroform-d) δ 9.90 (s, 1H), 8.10 (d, J=0.7 Hz, 1H), 8.06 (d, J=0.6 Hz, 1H), 4.75-4.63 (m, 2H), 3.70 (tdd, J=6.0, 1.4, 0.7 Hz, 2H), 2.66 (t, J=0.7 Hz, 3H). LCMS m/z 203.34 [M+H]⁺.

### Preparation S12

### 1-[3-[tert-butyl(dimethyl)silyl]oxy-2-[[tert-butyl(dimethyl)silyl]oxymethyl]-2-methyl-propyl]pyrazole-4-carbaldehyde (S12)

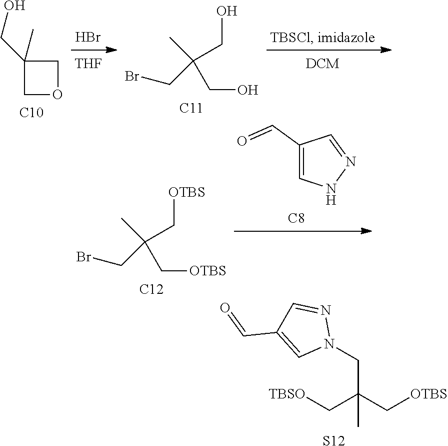

### Step 1: 2-(bromomethyl)-2-methyl-propane-1,3-diol (C11)

To a solution of (3-methyloxetan-3-yl)methanol C10 (10 mL, 100.3 mmol) in THF (70 mL) at 0° C. was added hydrogen bromide (14 mL of 48% w/w, 123.7 mmol) and stirred for 24 h. The mixture was concentrated and diluted in dichloromethane/methanol. Excess hydrogen bromide was quenched with saturated aqueous sodium bicarbonate. The layers were separated and the organic layer was dried over Na₂SO₄, filtered, rinsed with methanol and concentrated to give 2-(bromomethyl)-2-methyl-propane-1,3-diol C11 (13.6 g, 74%). ¹H NMR (400 MHz, Methanol-d4) δ 3.47 (d, J=1.1 Hz, 6H), 0.96 (s, 3H).

### Step 2: [2-(bromomethyl)-3-[tert-butyl(dimethyl)silyl]oxy-2-methyl-propoxy]-tert-butyl-dimethyl-silane (C12)

To a solution of 2-(bromomethyl)-2-methyl-propane-1,3-diol C11 (10 g, 54.1 mmol) in DCM (200 mL) was added imidazole (7.7 g, 113.1 mmol) followed by TBSCl (17 g, 112.8 mmol). After 5 min the solid was filtered and washed with DCM. The filtrate was diluted with heptane (25 mL) and the solid was filtered and washed with heptane (10 mL). The filtrate was concentrated to give [2-(bromomethyl)-3-[tert-butyl(dimethyl)silyl]oxy-2-methyl-propoxy]-tert-butyl-dimethyl-silane C12 (22.2 g, 99%). ¹H NMR (400 MHz, Chloroform-d) δ 3.44 (s, 4H), 3.40 (s, 2H), 0.94 (s, 3H), 0.89 (s, 18H), 0.04 (d, J=1.2 Hz, 12H).

### Step 3: 1-[3-[tert-butyl(dimethyl)silyl]oxy-2-[[tert-butyl(dimethyl)silyl]oxymethyl]-2-methyl-propyl]pyrazole-4-carbaldehyde (S12)

To a solution of 1H-pyrazole-4-carbaldehyde C8 (2 g, 20.8 mmol) in acetonitrile (20 mL) was added potassium carbonate (4 g, 28.9 mmol) and [2-(bromomethyl)-3-[tert-butyl(dimethyl)silyl]oxy-2-methyl-propoxy]-tert-butyl-dimethyl-silane C12 (9.5 g, 23.1 mmol) and stirred at 110° C. for 35 min. The mixture was cooled to rt, filtered, rinsed with acetonitrile and concentrated. Purification by silica gel chromatography (Gradient: 30-60% EtOAc in heptane) to give 1-[3-[tert-butyl(dimethyl)silyl]oxy-2-[[tert-butyl(dimethyl)silyl]oxymethyl]-2-methyl-propyl]pyrazole-4-carbaldehyde S12 (2.39 g, 23%). ¹H NMR (400 MHz, Chloroform-d) δ 9.85 (s, 1H), 7.98-7.91 (m, 2H), 4.12 (s, 2H), 3.43-3.29 (m, 4H), 0.91 (s, 18H), 0.84 (s, 3H), 0.05 (d, J=0.6 Hz, 12H). LCMS m/z 427.31 [M+H]⁺.

### Compound 264

### 2-[4-[[(2′S,4R)-2-ethyl-2′-methyl-spiro[6,7-dihydrothieno[3,2-c]pyran-4,4′-piperidine]-1′-yl]methyl]pyrazol-1-yl]-N-methyl-ethanesulfonamide

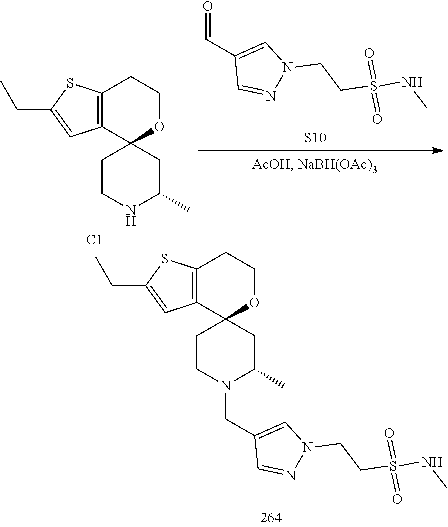

### Step 1: 2-[4-[[(2′S,4R)-2-ethyl-2′-methyl-spiro[6,7-dihydrothieno[3,2-c]pyran-4,4′-piperidine]-1′-yl]methyl]pyrazol-1-yl]-N-methyl-ethanesulfonamide (264)

A mixture of 2-(4-formylpyrazol-1-yl)-N-methyl-ethanesulfonamide S10 (100 mg, 0.460 mmol), (2′S)-2-ethyl-2′-methyl-spiro[6,7-dihydrothieno[3,2-c]pyran-4,4′-piperidine] C1 (100 mg, 0.338 mg), sodium acetoxyborohydride (250 mg, 1.18 mmol) and acetic acid (0.025 mL, 0.439 mmol) in 1,2-dichloroethane (5 mL) was stirred at 50° C. overnight. The mixture was diluted with saturated sodium bicarbonate and dichloromethane (10 mL) and the layers were separated. The organic layer was dried over Na₂SO₄, filtered and concentrated. Purification by silica gel chromatography (Gradient: 0-10% Methanol in DCM) gave 2-[4-[[(2′S,4R)-2-ethyl-2′-methyl-spiro[6,7-dihydrothieno[3,2-c]pyran-4,4′-piperidine]-1′-yl]methyl]pyrazol-1-yl]-N-methyl-ethanesulfonamide 264 (71 mg, 45%). ¹H NMR (400 MHz, Chloroform-d) δ 7.60-7.44 (m, 2H), 6.54-6.41 (m, 1H), 4.57 (t, J=6.0 Hz, 3H), 3.99-3.75 (m, 3H), 3.75-3.42 (m, 5H), 2.94-2.24 (m, 12H), 2.09-1.65 (m, 4H), 1.27 (t, J=7.5 Hz, 3H), 1.22 (d, J=6.2 Hz, 3H). LCMS m/zm/z 453.46 [M+H]⁺.

### Compounds 265-277

Compounds 265-277 (Table 6) were prepared from intermediate piperidines selected from C1, S5, S6, or S9, and appropriate aldehyde using the appropriate reagents as described in the method for compound 264. Aldehydes were prepared by methods described above or obtained from commercial sources. Any modifications to methods are noted in Table 6 and accompanying footnotes.

TABLE 6
Structure and physicochemical data for compounds 265-277

| | | | | ¹H NMR; LCMS |
|---|---|---|---|---|
| Cmpd | Structure | Piperidine | Aldehyde | m/zm/z [M + H]⁺ |
| 265 | 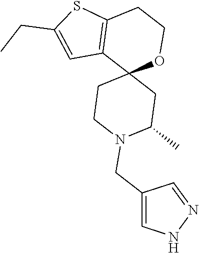 [SMILES: CCc1cc2c(s1)CCO[C@@]21CCN(Cc2cn[nH]c2)[C@@H](C)C1] | 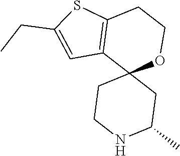 | 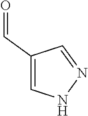 | LCMS m/zm/z 332.07 [M + H]⁺. |
| 266 | 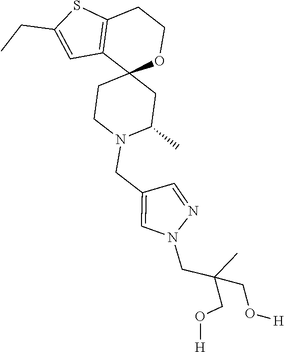 [SMILES: CCc1cc2c(s1)CCO[C@@]21CCN(Cc2cnn(CC(C)(CO)CO)c2)[C@@H](C)C1] | 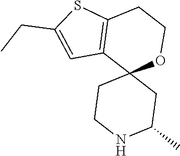 | 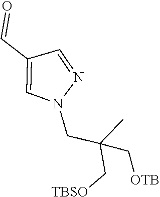 | ¹H NMR (400 MHz, Methanol- d4) δ 8.00 (d, J = 0.7 Hz, 1H), 7.76 (d, J = 0.7 Hz, 1H), 6.86-6.54 (m, 1H), 4.53 (d, J = 14.1 Hz, 1H), 4.29 (d, J = 14.1 Hz, 1H), 4.22 (s, 2H), 3.98-3.80 (m, 2H), 3.55- 3.33 (m, 5H), 3.29- 3.21 (m, 1H), 2.84-2.63 (m, 4H), 2.25-2.04 (m, 4H), 2.03 (s, 1H), 1.55 (d, J = 6.5 Hz, 3H), 1.25 (t, J = 7.5 Hz, 3H), 0.85 (s, 3H). LCMS m/z 433.87 [M + H]⁺. |
| 267 | 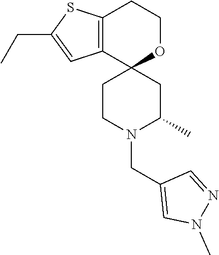 [SMILES: CCc1cc2c(s1)CCO[C@@]21CCN(Cc2cnn(C)c2)[C@@H](C)C1] | 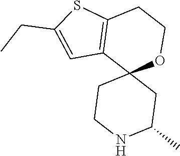 | 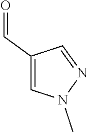 | ¹H NMR (300 MHz, Chloroform-d) δ 7.33 (d, J = 0.8 Hz, 1H), 7.21 (s, 1H), 6.40 (d, J = 1.1 Hz, 1H), 3.80- 3.69 (m, 3H), 3.49 (d, J = 14.2 Hz, 1H), 2.74-2.56 (m, 5H), 2.53- 2.35 (m, 2H), 1.89- 1.69 (m, 3H), 1.61 (dd, J = 13.8, 11.3 Hz, 1H), 1.18 (t, J = 7.5 Hz, 3H), 1.11 (d, J = 6.2 Hz, 3H). LCMS m/z 346.45 [M + H]⁺. |
| 268 | 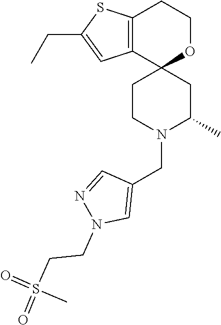 [SMILES: CCc1cc2c(s1)CCO[C@@]21CCN(Cc2cnn(CCS(C)(=O)=O)c2)[C@@H](C)C1] | 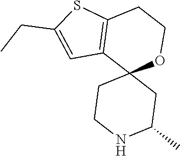 | 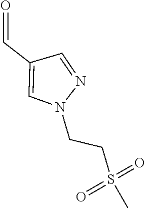 | ¹H NMR (400 MHz, Chloroform-d) δ 7.69-7.50 (m, 2H), 6.49 (q, J = 1.3 Hz, 1H), 4.68- 4.55 (m, 2H), 4.35 (s, 1H), 3.99- 3.75 (m, 3H), 3.75- 3.62 (m, 3H), 2.84-2.58 (m, 6H), 2.48 (d, J = 0.8 Hz, 3H), 2.04- 1.75 (m, 4H), 1.32- 1.18 (m, 6H). LCMS m/z 438.37 [M + H]⁺. |
| 269^{1,2} | 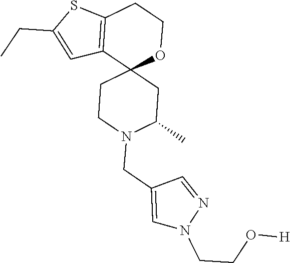 [SMILES: CCc1cc2c(s1)CCO[C@@]21CCN(Cc2cnn(CCO)c2)[C@@H](C)C1] | 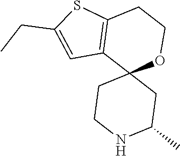 | 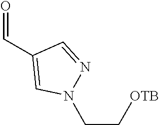 | ¹H NMR (400 MHz, Methanol- d4) δ 8.02 (d, J = 0.7 Hz, 1H), 7.80 (d, J = 0.7 Hz, 1H), 6.57-6.51 (m, 1H), 4.57 (d, J = 14.1 Hz, 1H), 4.30 (dd, J = 5.7, 4.8 Hz, 2H), 4.24 (d, J = 14.1 Hz, 1H), 3.94-3.86 (m, 4H), 3.54 (p, J = 7.0 Hz, 1H), 3.38-3.31 (m, 1H), 3.29-3.21 (m, 1H), 2.80- 2.70 (m, 4H), 2.22- 2.01 (m, 4H), 1.55 (d, J = 6.4 Hz, 3H), 1.25 (t, J = 7.5 Hz, 3H). LCMS m/z 375.74 [M + H]⁺. |
| 270¹ | 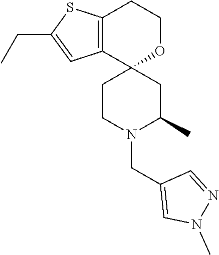 [SMILES: CCc1cc2c(s1)CCO[C@]21CCN(Cc2cnn(C)c2)[C@H](C)C1] | 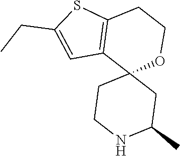 | 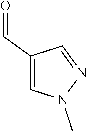 | LCMS m/z 346.47 [M + H]⁺. |
| 271¹ | 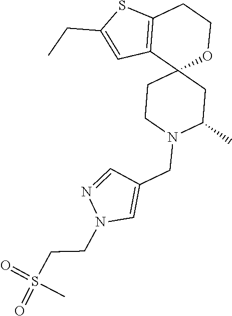 [SMILES: CCc1cc2c(s1)CCO[C@]21CCN(Cc2cnn(CCS(C)(=O)=O)c2)[C@@H](C)C1] | 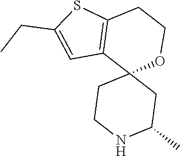 [SMILES: CCc1cc2c(s1)CCO[C@]21CCN[C@@H](C)C1] | 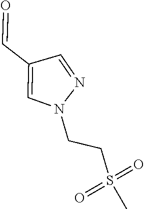 | ¹H NMR (300 MHz, Chloroform-d) δ 7.54 (d, J = 0.7 Hz, 1H), 7.53- 7.44 (m, 1H), 6.48 (d, J = 1.1 Hz, 1H), 4.71-4.50 (m, 2H), 4.01- 3.80 (m, 2H), 3.72- 3.58 (m, 2H), 3.54 (s, 2H), 3.05 (td, J = 5.7, 3.3 Hz, 1H), 2.89- 2.61 (m, 5H), 2.55- 2.40 (m, 4H), 2.03-1.87 (m, 2H), 1.87-1.72 (m, 2H), 1.26 (t, J = 7.5 Hz, 3H), 1.17 (d, J = 6.8 Hz, 3H). |
| 272¹ | 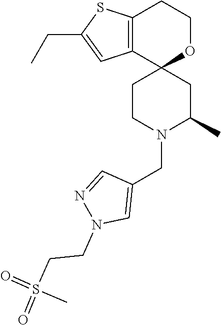 [SMILES: CCc1cc2c(s1)CCO[C@@]21CCN(Cc2cnn(CCS(C)(=O)=O)c2)[C@H](C)C1] | 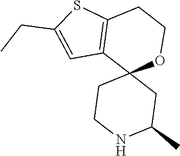 | 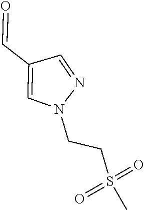 | ¹H NMR (300 MHz, Chloroform-d) δ 7.55 (s, 1H), 7.48 (s, 1H), 6.49 (s, 1H), 4.58 (dd, J = 6.9, 5.3 Hz, 2H), 3.98-3.81 (m, 2H), 3.64 (t, J = 6.1 Hz, 2H), 3.56 (s, 2H), 3.06 (s, 1H), 2.89-2.62 (m, 5H), 2.57- 2.39 (m, 1H), 2.47 (s, 3H), 1.96 (td, J = 14.0, 4.9 Hz, 2H), 1.89-1.71 (m, 2H), 1.26 (t, J = 7.5 Hz, 3H), 1.18 (d, J = 6.8 Hz, 3H). LCMS m/z 438.23 [M + H]⁺. |
| 273 | 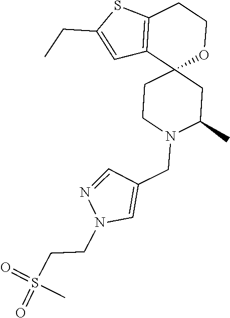 [SMILES: CCc1cc2c(s1)CCO[C@]21CCN(Cc2cnn(CCS(C)(=O)=O)c2)[C@H](C)C1] | 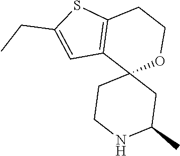 | 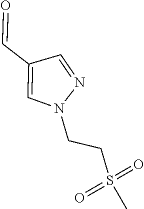 | LCMS m/z 438.37 [M + H]⁺. |
| 274¹ | 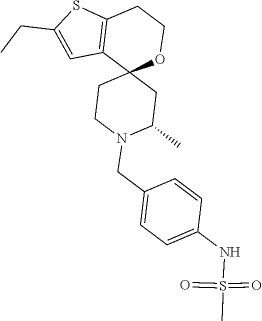 [SMILES: CCc1cc2c(s1)CCO[C@@]21CCN(Cc2ccc(NS(C)(=O)=O)cc2)[C@@H](C)C1] | 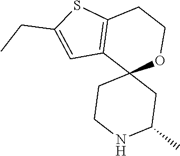 | 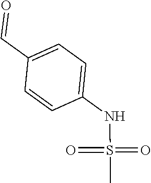 | LCMS m/z 435.42 [M+H]+ |
| 275¹ | 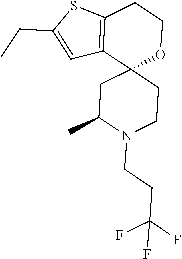 [SMILES: CCc1cc2c(s1)CCO[C@@]21CCN(CCC(F)(F)F)[C@@H](C)C1] | 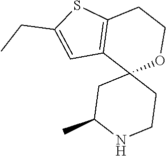 | 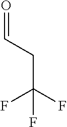 | ¹H NMR (400 MHz, DMSO-d6): δ 6.49 (s, 1H), 3.91-3.87 (m, 2H), 3.09-3.02 (m, 1H), 2.78-2.67 (m, 8H), 2.55-2.51 (m, 2H), 1.91-1.63 (m, 4H), 1.26-1.22 (t, J = 11.6 Hz, 3H), 1.10-1.09 (d, J = 6.0 Hz, 3H). LCMS m/z 348.53 [M + H]⁺. |
| 276¹ | 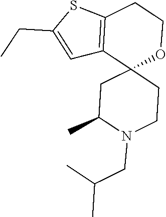 [SMILES: CCc1cc2c(s1)CCO[C@@]21CCN(CC(C)C)[C@@H](C)C1] | 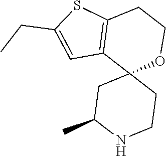 | 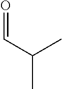 | ¹H NMR (400 MHz, DMSO-d6): δ 6.60 (s, 1H), 3.86-3.79 (m, 2H), 3.32 (m, 1H), 2.77-2.37 (m,7H), 1.94-1.58 (m, 6H), 1.21-1.17 (t, J = 7.6 Hz, 3H), 1.00- 0.99 (d, J = 6.4 Hz, 3H), 0.89-0.85 (t, J = 6.4 Hz, 6H). LCMS m/z 308.56 [M + H]⁺. |
| 277¹ | 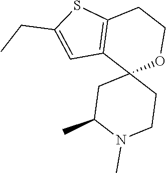 [SMILES: CCc1cc2c(s1)CCO[C@@]21CCN(C)[C@@H](C)C1] | 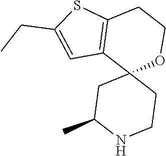 | 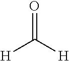 | ¹H NMR (400 MHz, DMSO-d6): δ 8.31 (s, 1H), 6.59 (s, 1H), 3.86- 3.85 (m, 2H), 3.04-2.88 (m,3H), 2.76-2.67 (m, 4H), 2.57 (s, 3H), 2.02- 1.74 (m, 4H), 1.23-1.14 (m, 6H). LCMS m/z 266.1 [M + H]⁺. |

Footnotes:
1) Reductive amination was run with 2 eq of polymer supported cyanoborohydride instead of sodium acetoxyborohydride. After completion of reductive amination product was stirred with aqeous HCI and MeOH for five minutes to deprotect the TBS group. White solid precipitated, was collected and dried to afford final product.

### Compound 278

### (2′S,4R)-2-ethyl-1′-(2-methoxyethyl)-2′-methyl-spiro[6,7-dihydrothieno[3,2-c]pyran-4,4′-piperidine]

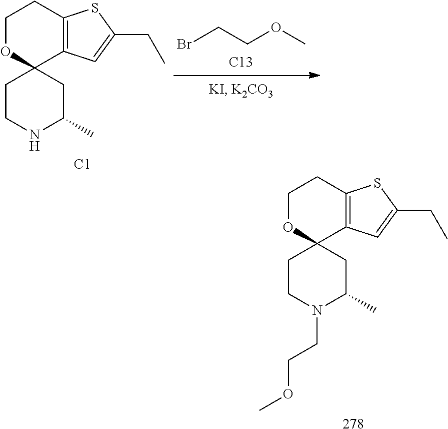

### Step 1: (2′S,4R)-2-ethyl-1′-(2-methoxyethyl)-2′-methyl-spiro[6,7-dihydrothieno[3,2-c]pyran-4,4′-piperidine](278)

To a stirred solution of (2′S,4R)-2-ethyl-2′-methyl-spiro[6,7-dihydrothieno[3,2-c]pyran-4,4′-piperidine] C1 (100 mg, 0.236 mmol) and 1-bromo-2-methoxy-ethane C13 (99 mg, 0.712 mmol) in MeCN (8 mL) in DMF (1 mL) was added potassium carbonate (180 mg, 13.0 mmol) followed by KI (8 mg, 0.048 mmol) and stirred at 60° C. for 48 h. The mixture was diluted with MeCN (20 mL) and filtered. The filtrate was concentrated and purified by Prep-HPLC (conditions: mobile phase A: −0.01 M Ammonium bicarbonate (Aq), mobile phase B: -acetonitrile) and pure fractions collected to give (2′S,4R)-2-ethyl-1′-(2-methoxyethyl)-2′-methyl-spiro[6,7-dihydrothieno[3,2-c]pyran-4,4′-piperidine]278 (28 mg, 38%). ¹H NMR (400 MHz, DMSO-d6): δ 6.60 (s, 1H), 3.83-3.79 (q, J=5.2 Hz, 2H), 3.42-3.39 (t, J=6.0 Hz, 2H), 3.23 (s, 3H), 2.89-2.86 (m, 1H), 2.73-2.63 (m, 5H), 2.54-2.32 (m, 3H), 1.70-1.49 (m, 4H), 1.20-1.17 (t, J=7.6 Hz, 3H), 0.98-0.97 (d, J=6.0 Hz, 3H). LCMS m/z 310.2 [M+H]⁺.

### Compound 279

### (2′S,4R)-2-ethyl-1′-(isoxazol-3-ylmethyl)-2′-methyl-spiro[6,7-dihydrothieno[3,2-c]pyran-4,4′-piperidine]

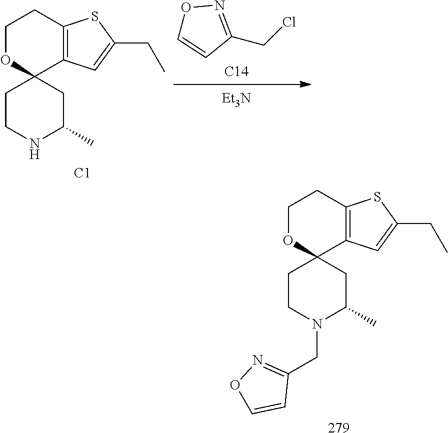

### Step 1: (2′S,4R)-2-ethyl-1′-(isoxazol-3-ylmethyl)-2′-methyl-spiro[6,7-dihydrothieno[3,2-c]pyran-4,4′-piperidine](279)

To a solution of (2′S,4R)-2-ethyl-2′-methyl-spiro[6,7-dihydrothieno[3,2-c]pyran-4,4′-piperidine] C1 (100 mg, 0.397 mmol) in DMF (2 mL) was added triethylamine (0.16 mL, 1.19 mmol) followed by 3-(chloromethyl)isoxazole C14 (56 mg, 0.477 mmol). The mixture was stirred at room temperature overnight. Diluted with EtOAc (20 mL), washed with water (2×10 mL) and brine (2×5 mL). The organic layer was dried over anhydrous Na₂SO₄, filtered and concentrated. Purification by Prep-HPLC gave (2′S,4R)-2-ethyl-1′-(isoxazol-3-ylmethyl)-2′-methyl-spiro[6,7-dihydrothieno[3,2-c]pyran-4,4′-piperidine]279 (9 mg, 7%). ¹H NMR (400 MHz, DMSO-d6) 8.85 (s, 1H), 6.60 (s, 1H), 6.52 (s, 1H), 3.91 (d, J=14.55 Hz, 2H), 3.80-3.76 (m, 2H), 3.63 (d, J=14.36 Hz, 1H), 2.70 (q, J=7.44 Hz, 14.92 Hz 2H), 2.62 (bs, 2H), 2.49-2.43 (m, 2H), 1.77-1.69 (m, 3H), 1.58 (t, J=12.16 Hz 1H), 1.19 (t, J=7.44 Hz, 3H), 1.10 (d, J=6.08 Hz, 3H). LCMS m/z 333.0 [M+H]⁺.

### Compounds 280-298

Compounds 280-298 were prepared from intermediate C1 and corresponding commercial alkyl halides using methods as described for compounds 278 and 279. Any modifications to methods are noted in Table 7 and accompanying footnotes.

TABLE 7
Structure and physicochemical data for compounds 280-298

| | | | Alkyl | ¹H NMR; |
|---|---|---|---|---|
| Cmpd | Structure | Method | halide | LCMS m/z [M + H]⁺ |
| 280 | 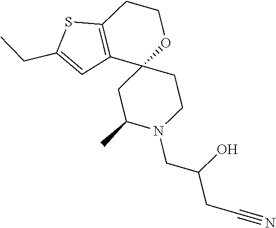 [SMILES: CCc1cc2c(s1)CCO[C@@]21CCN(CC(O)CC#N)[C@@H](C)C1] | 2781 | 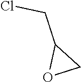 | ¹H NMR (400 MHz, DMSO-d6) δ 6.58 (s, 1H), 5.12 (br s, 1H), 3.85-3.78 (m, 3H), 2.73-2.54 (m, 10H), 2.16-2.12 (m, 1H), 1.82-1.79 (m, 1H), 1.66 (br d, J = 13.2 Hz, 2H), 1.51 (t, J = 12.4 Hz, 1H), 1.19 (t, J = 7.2 Hz, 3H), 0.97 (d, J = 6 Hz, 3H). LCMS m/z 335.2 [M + H]⁺. |
| 281 | 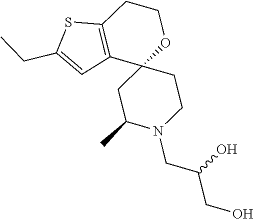 [SMILES: CCc1cc2c(s1)CCO[C@@]21CCN(CC(O)CO)[C@@H](C)C1] | 278 | 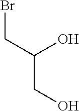 | ¹H NMR (400 MHz, DMSO-d6): δ 6.59 (s, 1H), 4.55 (s, 1H), 4.22 (s, 1H), 3.84-3.79 (m, 2H), 3.57 (m, 1H), 3.31 (m, 2H), 2.73- 2.51 (m, 8H), 2.24-2.20 (m, 1H), 1.78-1.48 (m, 4H), 1.21-1.17 (t, J = 7.6 Hz, 3H), 0.98-0.97 (t, J = 6.0 Hz, 3H). LCMS m/z 326.17 [M + H]⁺. |
| 282 | 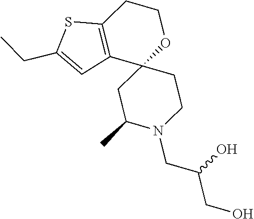 [SMILES: CCc1cc2c(s1)CCO[C@@]21CCN(CC(O)CO)[C@@H](C)C1] | 278 | 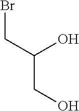 | ¹H NMR (400 MHz, DMSO-d6): δ 6.59 (s, 1H), 4.40-4.62 (m, 2H), 3.84- 3.80 (m, 2H), 3.57 (m, 1H), 3.41-3.27 (m, 2H), 2.79- 2.64 (m, 6H), 2.50-2.45 (m, 2H), 2.07 (m, 1H), 1.81- 1.49 (m, 4H), 1.21-1.17 (t, J = 7.6 Hz, 3H), 0.98-0.97 (t, J = 6.4 Hz, 3H). LCMS m/z 326.17 [M + H]⁺. |
| 283 | 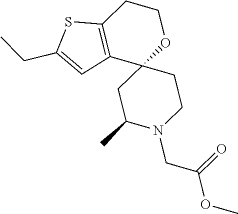 [SMILES: CCc1cc2c(s1)CCO[C@@]21CCN(CC(=O)OC)[C@@H](C)C1] | 278 | 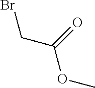 | ¹H NMR (400 MHz, DMSO-d6): δ 6.61 (s, 1H), 3.84-3.80 (q, J = 5.6 Hz, 2H), 3.61 (s, 3H), 3.45-3.41 (m, 1H), 3.32-3.27 (m, 1H), 2.80-2.60 (m, 7H), 1.80- 1.47 (m, 4H), 1.21-1.17 (t, J = 7.2 Hz, 3H), 0.96-0.94 (d, J = 6.4 Hz, 3H). LCMS m/z 324.49 [M + H]⁺. |
| 284 | 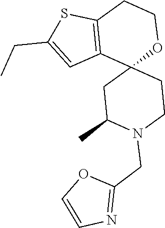 [SMILES: CCc1cc2c(s1)CCO[C@@]21CCN(Cc2ncco2)[C@@H](C)C1] | 278 | 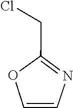 | ¹H NMR (400 MHz, DMSO-d6) δ 8.072 (d, J = 0.8, 1H), 7.17 (d, J = 0.8, 1H), 6.608 (s, 1H), 3.89- 3.74 (m, 4H), 2.73-2.67 (m, 2H), 2.63-2.53 (m, 5H), 1.80-1.75 (m, 1H), 1.685 (d, J =13.6, 2H), 1.54 (t, J = 11.2 Hz, 1H), 1.18 (t, J = 7.6 Hz, 3H), 1.095 (d, J = 6.4 Hz, 3H). LCMS m/z 333.2 [M + H]⁺. |
| 285 | 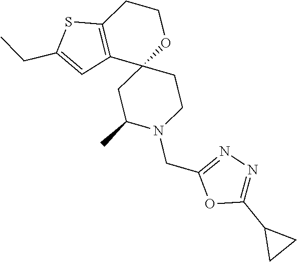 [SMILES: CCc1cc2c(s1)CCO[C@@]21CCN(Cc2nnc(C3CC3)o2)[C@@H](C)C1] | 278 | 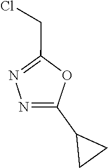 | ¹H NMR (400 MHz, DMSO-d6) δ 6.61 (s, 1H), 3.915 (q, J = 15.6, Hz, 2H), 3.81-3.77 (m, 2H), 2.70 (q, J = 7.6, Hz, 2H), 2.64-2.59 (m, 5H), 2.24-2.22 (m, 1H), 1.85-1.75 (m, 1H), 1.70 (d, J = 14, 2H) , 1.55 (t, J = 11.2 Hz, 1H), 1.18 (t, J = 7.6 Hz, 3H), 1.15-1.11 (m, 2H), 1.07 (d, J = 6 Hz, 3H), 0.99- 0.96 (m, 2H). LCMS m/z 374.2 [M + H]⁺. |
| 286 | 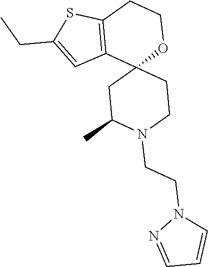 [SMILES: CCc1cc2c(s1)CCO[C@@]21CCN(CCn2cccn2)[C@@H](C)C1] | 278 | 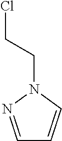 | ¹H NMR (400 MHz, DMSO-d6) δ 7.742 (d, J = 2, 1H), 7.415 (d, J = 1.2, 1H), 6.59 (s, 1H), 6.20 (t, J = 2 Hz, 1H), 4.18 (t, J = 7.2 Hz, 2H) 3.83-3.80 (m, 2H), 3.12-3.08 (m, 1H), 2.73- 2.56 (m, 8H), 1.78-1.65 (m, 3H), 1.47 (t, J = 11.6 Hz, 1H), 1.19 (t, J = 7.2 Hz, 3H), 0.928 (d, J = 6 Hz, 3H). LCMS m/z 346.1 [M + H]⁺. |
| 287 | 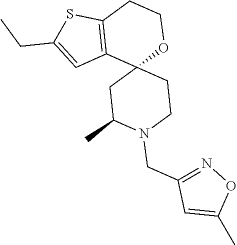 [SMILES: CCc1cc2c(s1)CCO[C@@]21CCN(Cc2cc(C)on2)[C@@H](C)C1] | 278 | 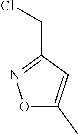 | ¹H NMR (400 MHz, DMSO-d6) δ 6.59 (s, 1H), 6.16 (s, 1H), 3.84-3.75 (m, 3H), 3.50 (d, J = 14.4 Hz, 1H), 2.70 (q, J = 7.44 Hz, J = 14.92 Hz, 2H), 2.62 (bs,2H), ~2.50 (2H, under DMSO) 2.38 (s, 3H), 1.76- 1.69 (m, 3H), 1.54 (t, J = 11.72 Hz 1H), 1.18 (t, J = 7.52 Hz, 3H), 1.08 (d, J = 6.1 Hz, 3H). LCMS m/z 347.0 [M + H]⁺. |
| 288 | 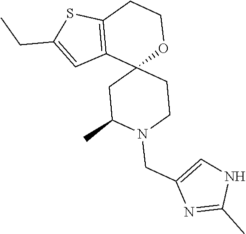 [SMILES: CCc1cc2c(s1)CCO[C@@]21CCN(Cc2c[nH]c(C)n2)[C@@H](C)C1] | 278 | 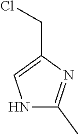 | ¹H NMR (400 MHz, DMSO-d6) δ 11.43 (bs, 1H), 6.66 (bs, 1H), 6.56 (s, 1H), 3.81-3.68 (m, 3H), 3.37-3.31 (m, 1H), 2.70 (q, J = 7.52 Hz, 15 Hz, 2H), 2.61-2.56 (m, 3H), 2.50- 2.32 (m, 2H), 2.22 (s, 3H), 1.74-1.66 (m,3H), 1.53 (t, J = 11.68 Hz, 1H), 1.18 (t, J = 7.48 Hz, 3H), 1.11 (d, J = 6.08 Hz, 3H). LCMS m/z 346.0 [M + H]⁺. |
| 289 | 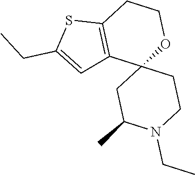 [SMILES: CCc1cc2c(s1)CCO[C@@]21CCN(CC)[C@@H](C)C1] | 278 | 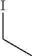 | ¹H NMR (400 MHz, DMSO-d6): δ 8.25 (s, 1H), 6.59 (s, 1H), 3.86-3.49 (m, 2H), 2.84-2.81 (t, J = 6.4 Hz, 1H), 2.73-2.60 (m, 6H), 2.50-2.52 (m, 1H), 1.84- 1.58 (m, 4H), 1.21-1.17 (t, J = 7.2 Hz, 3 H), 1.01-0.96 (m, 6H). LCMS m/z 280.1 [M + H]⁺. |
| 290 | 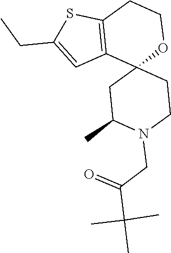 [SMILES: CCc1cc2c(s1)CCO[C@@]21CCN(CC(=O)C(C)(C)C)[C@@H](C)C1] | 278 | 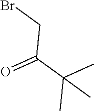 | ¹H NMR (400 MHz, DMSO-d6): δ 6.61 (s, 1H), 3.84-3.69 (m, 3H), 3.38- 3.33 (m, 1H), 2.82-2.58 (m, 7H), 1.77-1.33 (m, 4H), 1.21-1.17 (t, J = 7.2 Hz, 3H), 1.09 (s, 9H), 0.93-0.92 (d, J = 6.4 Hz, 3H). LCMS m/z 351.1 [M + H]⁺. |
| 291 | 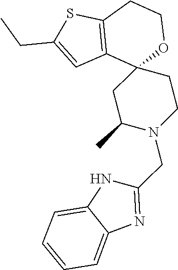 [SMILES: CCc1cc2c(s1)CCO[C@@]21CCN(Cc2nc3ccccc3[nH]2)[C@@H](C)C1] | 278 | 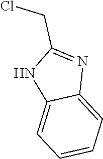 [SMILES: ClCc1nc2ccccc2[nH]1] | ¹H NMR (400 MHz, DMSO-d6) δ 12.14 (br s, 1H), 7.50 (br s, 2H), 7.14- 7.12 (m, 2H), 6.56 (s, 1H), 4.06(d, J = 14.4 Hz, 1H), 3.81-3.76 (m, 2H), 3.65 (d, J = 14.8 Hz, 1H), 2.74-2.51 (m, 7H), 1.84-1.80 (m, 1H), 1.75-1.58 (m, 3H), 1.19 (t, J = 7.6 Hz, 3H), 1.14 (d, J = 6.4 Hz, 3H). LCMS m/z 382.2 [M + H]⁺. |
| 292 | 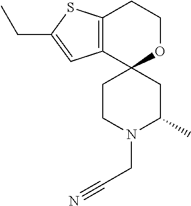 [SMILES: CCc1cc2c(s1)CCO[C@@]21CCN(CC#N)[C@@H](C)C1] | 278 | 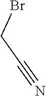 | ¹H NMR (400 MHz, DMSO-d6): δ 6.60 (s, 1H), 3.95-3.83 (m, 3H), 3.71- 3.67 (d, J = 16 Hz, 1H), 2.73-2.57 (m, 7H), 1.85- 1.52 (m, 4H), 1.21-1.17 (t, J = 7.2 Hz, 3H), 0.99-0.98 (d, J = 6.4 Hz, 3H). LCMS m/z 291.26 [M + H]⁺. |
| 293 | 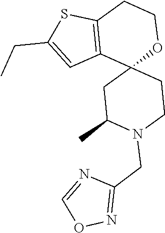 [SMILES: CCc1cc2c(s1)CCO[C@@]21CCN(Cc2ncon2)[C@@H](C)C1] | 278 | 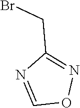 | ¹H NMR (400 MHz, DMSO-d6) δ 9.56 (s, 1H), 6.60 (s, 1H), 3.93-3.92 (m, 2H), 3.79-3.75 (m, 2H), 2.73-2.67 (m, 2H), 2.62- 2.60 (m, 5H), 1.81-1.75 (m, 1H), 1.69 (d, J = 13.2 Hz, 2H), 1.58-1.52 (m, 1H), 1.20 (t, J = 10.96 Hz, 3H), 1.10 (d, J = 6.12 Hz, 3H). LCMS m/z 334.0 [M + H]⁺. |
| 294 | 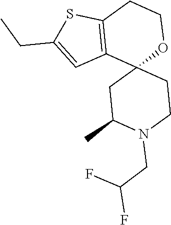 [SMILES: CCc1cc2c(s1)CCO[C@@]21CCN(CC(F)F)[C@@H](C)C1] | 2782 | 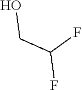 | ¹H NMR (400 MHz, DMSO-d6): δ 6.62 (s, 1H), 6.22-5.92 (tt, J = 16.0, 4.4 Hz, 1H), 3.86-3.79 (m, 2H), 3.08-3.03 (m, 1H), 2.76- 2.54 (m, 8H), 1.80-1.48 (m, 4H), 1.21-1.17 (t, J = 7.2 Hz, 3H), 0.98-0.97 (d, J = 6.4 Hz, 3H). LCMS m/z 316.24 [M + H]⁺. |
| 295 | 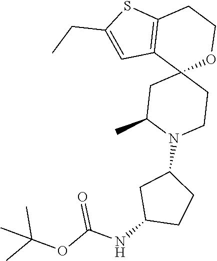 [SMILES: CCc1cc2c(s1)CCO[C@@]21CCN([C@@H]2CC[C@H](NC(=O)OC(C)(C)C)C2)[C@@H](C)C1] | 264 | 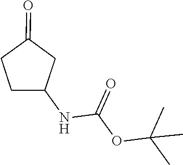 | LCMS m/z 435.53 [M + H]⁺. |
| 296 | 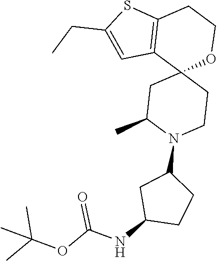 | 264 | 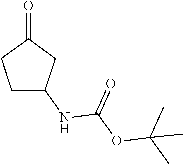 | LCMS m/z 435.17 [M + H]⁺. |
| 297 | 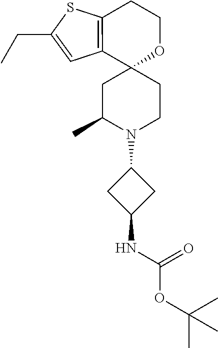 [SMILES: CCc1cc2c(s1)CCO[C@@]21CCN([C@H]2C[C@H](NC(=O)OC(C)(C)C)C2)[C@@H](C)C1] | 264 | 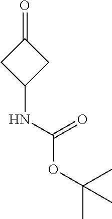 | LCMS m/z 421.36 [M + H]⁺. |
| 298 | 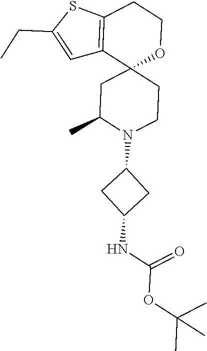 [SMILES: CCc1cc2c(s1)CCO[C@@]21CCN([C@H]2C[C@@H](NC(=O)OC(C)(C)C)C2)[C@@H](C)C1] | 264 | 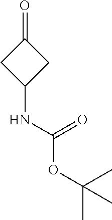 | LCMS m/z 421.36 [M + H]⁺. |

Footnotes:
1) Epoxide opening was run using NaCN in DMF
2) alcohol was stirred with trifluoromethanesulfonic anhydride at 80° C. then added to reaction mixture
3) Compounds 295-298 stereoisomers were separated by chiral SFC purification

### Preparation of Compound 299

### 3-[[(2′S,4R)-2-ethyl-2′-methyl-spiro[6,7-dihydrothieno[3,2-c]pyran-4,4′-piperidine]-1′-yl]methyl]cyclobutanamine (299)

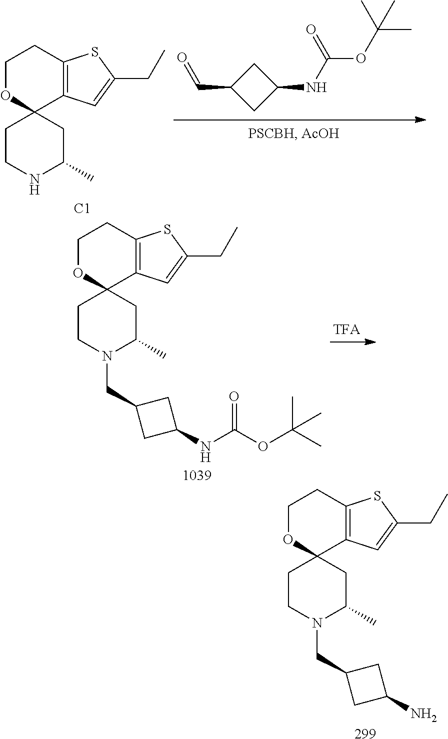

### Step 1. Synthesis of tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (1039)

A microwave tube was charged with (2′S,4R)-2-ethyl-2′-methyl-spiro[6,7-dihydrothieno[3,2-c]pyran-4,4′-piperidine]trifluoromethanesulfonate (C1) (2.468 g, 6.163 mmol), tert-butyl N-(3-formylcyclobutyl)carbamate (6.54 g, 32.82 mmol), polymer-supported cyanoborohydride (11.4 g of 2 mmol/g, 22.80 mmol), and acetic acid (3 mL, 52.75 mmol) in DCM (45 mL). The reaction was heated to 110° C. for 30 minutes under microwave irradiation. After cooling to room temperature, the reaction was filtered, and the filtrate was diluted with DCM (150 mL). The organic phase was washed with 1 N NaOH (2×100 mL), dried over Na₂SO₄, filtered, and evaporated in vacuo. Purification by silica gel chromatography (120 g column, 0-5% MeOH in DCM) afforded the product tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (1042) (2.170 g, 79%). ¹H NMR (300 MHz, Chloroform-d) δ 6.49 (s, 1H), 4.84-4.45 (bs, 1H), 4.17-3.76 (m, 3H), 2.90-2.72 (m, 5H), 2.69-2.33 (m, 6H), 2.28-2.15 (m, 1H), 2.00-1.76 (m, 3H), 1.76-1.62 (m, 1H), 1.60-1.45 (m, 2H), 1.45 (s, 9H), 1.28 (t, J=7.5 Hz, 3H), 1.08 (d, J=6.2 Hz, 3H). ss LCMS m/z 435.4 [M+H]⁺.

### Step 3. Synthesis of 3-[[(2′S,4R)-2-ethyl-2′-methyl-spiro[6,7-dihydrothieno[3,2-c]pyran-4,4′-piperidine]-1′-yl]methyl]cyclobutanamine (299)

tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (1042) (2.04 g, 4.600 mmol) was dissolved in a 4 M solution of HCl in dioxane (10 mL, 40.00 mmol), and the reaction was allowed to stir at room temperature for 2 hours. The reaction was concentrated in vacuo to give crude 3-[[(2′S,4R)-2-ethyl-2′-methyl-spiro[6,7-dihydrothieno[3,2-c]pyran-4,4′-piperidine]-1′-yl]methyl]cyclobutanamine (Hydrochloride salt) (299) (1.7 g, 96%). LCMS m/z 334.68 [M+H]⁺.

### Compound 300

### N-[3-[[(2′S,4R)-2-ethyl-2′-methyl-spiro[6,7-dihydrothieno[3,2-c]pyran-4,4′-piperidine]-1′-yl]methyl]cyclobutyl]-3,3-difluoro-cyclobutanecarboxamide (300)

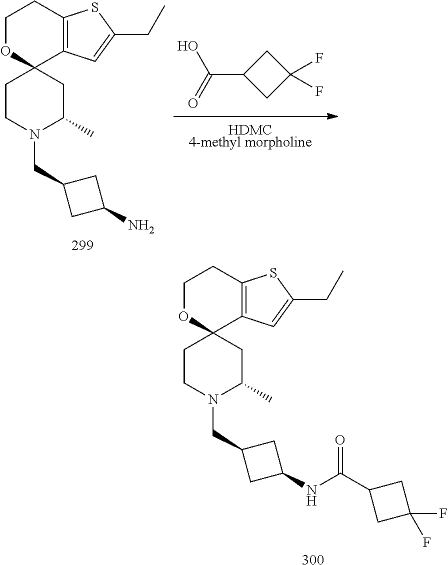

### Preparation of N-[3-[[(2′S,4R)-2-ethyl-2′-methyl-spiro[6,7-dihydrothieno[3,2-c]pyran-4,4′-piperidine]-1′-yl]methyl]cyclobutyl]-3,3-difluoro-cyclobutanecarboxamide (300)

3-[[(2′S,4R)-2-ethyl-2′-methyl-spiro[6,7-dihydrothieno[3,2-c]pyran-4,4′-piperidine]-1′-yl]methyl]cyclobutanamine (Hydrochloride salt) 299 (20 mg, 0.05979 mmol) and 3,3-difluorocyclobutanecarboxylic acid (8.1 mg, 0.05979 mmol) were dissolved in DMF to which HDMC (32.7 mg, 0.07175 mmol) and 4-methylmorpholine (20 μL, 0.1794 mmol) were added. The reaction was stirred at room temperature for 1 hour. Purification by reversed-phase HPLC. Method: C18 Waters Sunfire column (30×150 mm, 5 micron). Gradient: MeCN in H₂O with 0.1% trifluoroacetic acid. afforded the product N-[3-[[(2′S,4R)-2-ethyl-2′-methyl-spiro[6,7-dihydrothieno[3,2-c]pyran-4,4′-piperidine]-1′-yl]methyl]cyclobutyl]-3,3-difluoro-cyclobutanecarboxamide (Trifluoroacetic acid salt) (300) (17 mg, 50%). LCMS m/z 453.24 [M+H]⁺.

### Compound 301

### N-[3-[[(2′S,4R)-2-ethyl-2′-methyl-spiro[6,7-dihydrothieno[3,2-c]pyran-4,4′-piperidine]-1′-yl]methyl]cyclobutyl]-2-(2,2,2-trifluoroethoxy)acetamide (301)

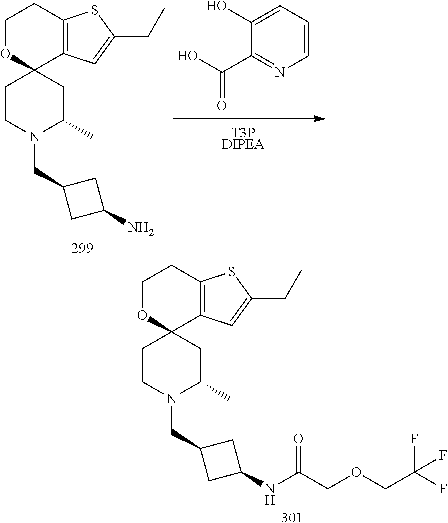

### Preparation of N-[3-[[(2′S,4R)-2-ethyl-2′-methyl-spiro[6,7-dihydrothieno[3,2-c]pyran-4,4′-piperidine]-1′-yl]methyl]cyclobutyl]-2-(2,2,2-trifluoroethoxy)acetamide (301)

3-[[(2′S,4R)-2-ethyl-2′-methyl-spiro[6,7-dihydrothieno[3,2-c]pyran-4,4′-piperidine]-1′-yl]methyl]cyclobutanamine (Hydrochloride salt) 299 (20 mg, 0.05979 mmol) and 2-(2,2,2-trifluoroethoxy)acetic acid (9.5 mg, 0.05979 mmol) were dissolved in DMF (1 mL), to which T3P (38.5 mg, 0.1210 mmol) and) and DIPEA (21 μL, 0.1206 mmol) were added. The reaction was allowed to stir at room temperature overnight. Purification by reversed-phase HPLC. Method: C18 Waters Sunfire column (30×150 mm, 5 micron). Gradient: MeCN in H₂O with 0.1% trifluoroacetic acid afforded N-[3-[[(2′S,4R)-2-ethyl-2′-methyl-spiro[6,7-dihydrothieno[3,2-c]pyran-4,4′-piperidine]-1′-yl]methyl]cyclobutyl]-2-(2,2,2-trifluoroethoxy)acetamide (Trifluoroacetic acid salt) (6.8 mg, 24%) (301). LCMS m/z 475.19 [M+H]⁺.

### Compound 302

### methyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (302)

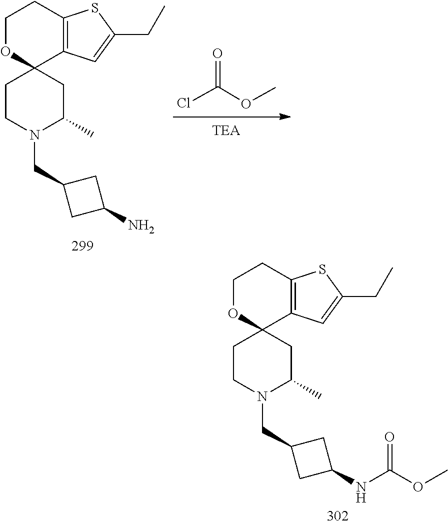

### Preparation of methyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (302)

3-[[(2′S,4R)-2-ethyl-2′-methyl-spiro[6,7-dihydrothieno[3,2-c]pyran-4,4′-piperidine]-1′-yl]methyl]cyclobutanamine (Hydrochloride salt) 299 (18 mg, 0.03962 mmol) was dissolved in DCM (1 mL), to which methyl carbonochloridate (5 μL, 0.06471 mmol) and triethylamine (15 μL, 0.1076 mmol) were added. The reaction was stirred at room temperature for 2 hours, after which time the solvent was evaporated in vacuo. Purification by reversed-phase HPLC. Method: C18 Waters Sunfire column (30×150 mm, 5 micron). Gradient: MeCN in H₂O with 0.1% trifluoroacetic acid afforded the product methyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (Trifluoroacetic acid salt) (302) (9.7 mg, 45%). LCMS m/z 393.23 [M+H]⁺.

### Compounds 303-384

Compounds 303-384 (see Table 8) were prepared from intermediate 299 using the appropriate reagent and using the amide formation methods as described for compounds 300-302. Coupling partners were obtained from commercial sources. Any modifications to methods are noted in Table 8 and accompanying footnotes.

TABLE 8
Method of preparation, structure and physicochemical data for compounds 303-384

| | | Amide | | |
|---|---|---|---|---|
| | | Coupling | | ¹H NMR; LCMS |
| Cmpd | Structure | Partner | Method | m/z [M + H]⁺ |
| 303 | 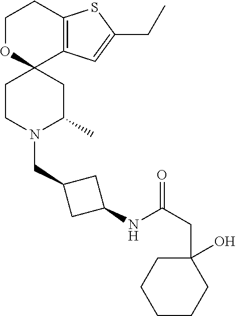 [SMILES: CCc1cc2c(s1)CCO[C@@]21CCN(C[C@H]2C[C@@H](NC(=O)CC3(O)CCCCC3)C2)[C@@H](C)C1] | 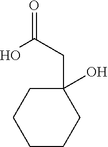 [SMILES: O=C(O)CC1(O)CCCCC1] | Compound 300 | LCMS m/z 475.32 [M + H]⁺. |
| 304 | 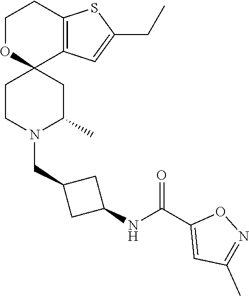 [SMILES: CCc1cc2c(s1)CCO[C@@]21CCN(C[C@H]2C[C@@H](NC(=O)c3cc(C)no3)C2)[C@@H](C)C1] | 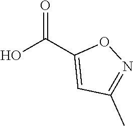 | Compound 300 | LCMS m/z 444.18 [M + H]⁺. |
| 305 | 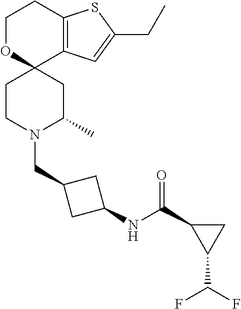 [SMILES: CCc1cc2c(s1)CCO[C@@]21CCN(C[C@H]2C[C@@H](NC(=O)[C@H]3C[C@@H]3C(F)F)C2)[C@@H](C)C1] | 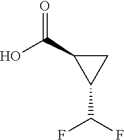 | Compound 300 | LCMS m/z 453.20 [M + H]⁺. |
| 306 | 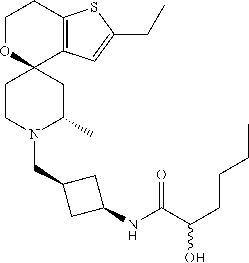 [SMILES: CCCCC(O)C(=O)N[C@H]1C[C@@H](CN2CC[C@]3(C[C@@H]2C)OCCc2sc(CC)cc23)C1] | 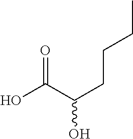 | Compound 300 | LCMS m/z 449.30 [M + H]⁺. |
| 307 | 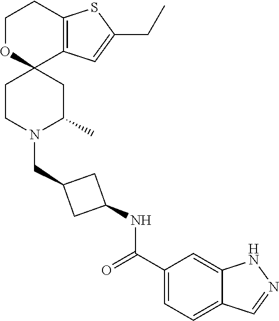 [SMILES: CCc1cc2c(s1)CCO[C@@]21CCN(C[C@H]2C[C@@H](NC(=O)c3ccc4cn[nH]c4c3)C2)[C@@H](C)C1] | 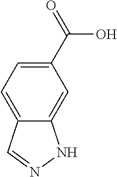 [SMILES: O=C(O)c1ccc2cn[nH]c2c1] | Compound 300 | LCMS m/z 479.23 [M + H]⁺. |
| 308 | 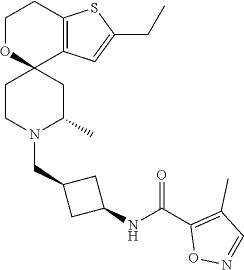 [SMILES: CCc1cc2c(s1)CCO[C@@]21CCN(C[C@H]2C[C@@H](NC(=O)c3oncc3C)C2)[C@@H](C)C1] | 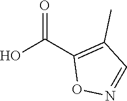 | Compound 300 | LCMS m/z 444.21 [M + H]⁺. |
| 309 | 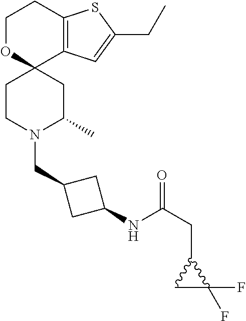 | 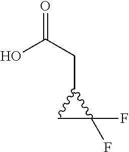 | Compound 300 | LCMS m/z 453.20 [M + H]⁺. |
| 310 | 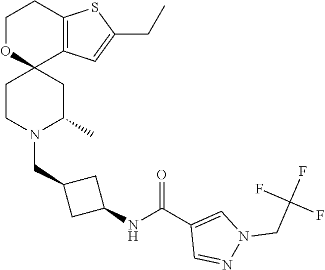 [SMILES: CCc1cc2c(s1)CCO[C@@]21CCN(C[C@H]2C[C@@H](NC(=O)c3cnn(CC(F)(F)F)c3)C2)[C@@H](C)C1] | 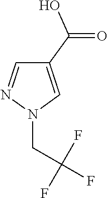 [SMILES: O=C(O)c1cnn(CC(F)(F)F)c1] | Compound 300 | LCMS m/z 455.19 [M + H]⁺. |
| 311 | 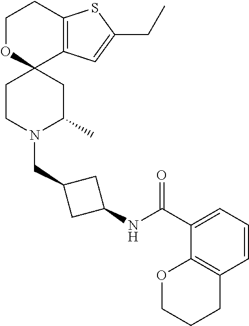 [SMILES: CCc1cc2c(s1)CCO[C@@]21CCN(C[C@H]2C[C@@H](NC(=O)c3cccc4c3OCCC4)C2)[C@@H](C)C1] | 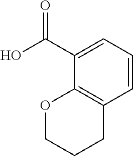 [SMILES: O=C(O)c1cccc2c1OCCC2] | Compound 300 | LCMS m/z 495.10 [M + H]⁺. |
| 312 | 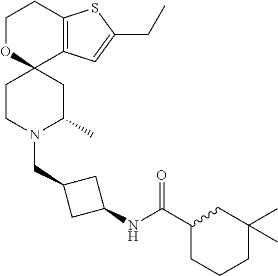 [SMILES: CCc1cc2c(s1)CCO[C@@]21CCN(C[C@H]2C[C@@H](NC(=O)C3CCCC(C)(C)C3)C2)[C@@H](C)C1] | 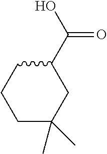 | Compound 300 | LCMS m/z 473.30 [M + H]⁺. |
| 313 | 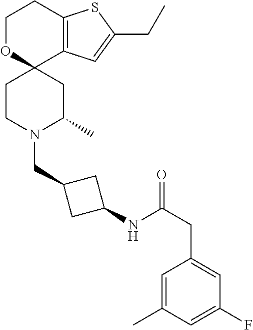 [SMILES: CCc1cc2c(s1)CCO[C@@]21CCN(C[C@H]2C[C@@H](NC(=O)Cc3cc(C)cc(F)c3)C2)[C@@H](C)C1] | 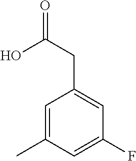 | Compound 300 | LCMS m/z 485.23 [M + H]⁺. |
| 314 | 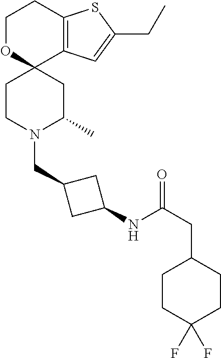 [SMILES: CCc1cc2c(s1)CCO[C@@]21CCN(C[C@H]2C[C@@H](NC(=O)CC3CCC(F)(F)CC3)C2)[C@@H](C)C1] | 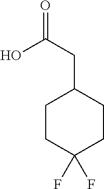 [SMILES: O=C(O)CC1CCC(F)(F)CC1] | Compound 300 | LCMS m/z 495.29 [M + H]⁺. |
| 315 | 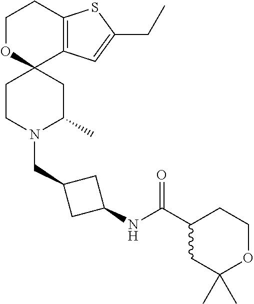 [SMILES: CCc1cc2c(s1)CCO[C@@]21CCN(C[C@H]2C[C@@H](NC(=O)C3CCOC(C)(C)C3)C2)[C@@H](C)C1] | 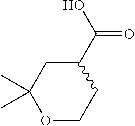 | Compound 300 | LCMS m/z 475.29 [M + H]⁺. |
| 316 | 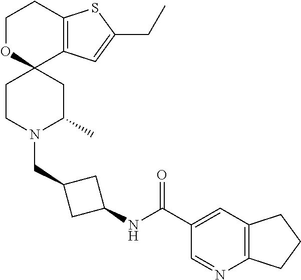 [SMILES: CCc1cc2c(s1)CCO[C@@]21CCN(C[C@H]2C[C@@H](NC(=O)c3cnc4c(c3)CCC4)C2)[C@@H](C)C1] | 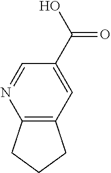 [SMILES: O=C(O)c1cnc2c(c1)CCC2] | Compound 300 | LCMS m/z 480.24 [M + H]⁺. |
| 317 | 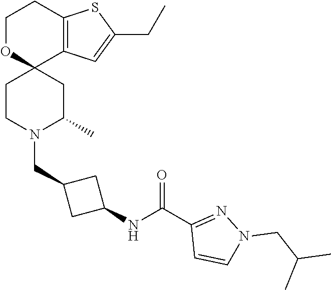 [SMILES: CCc1cc2c(s1)CCO[C@@]21CCN(C[C@H]2C[C@@H](NC(=O)c3ccn(CC(C)C)n3)C2)[C@@H](C)C1] | 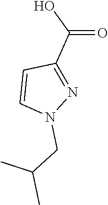 | Compound 300 | LCMS m/z 485.29 [M + H]⁺. |
| 318 | 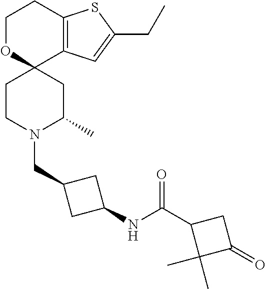 [SMILES: CCc1cc2c(s1)CCO[C@@]21CCN(C[C@H]2C[C@@H](NC(=O)C3CC(=O)C3(C)C)C2)[C@@H](C)C1] | 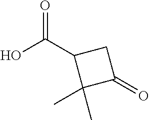 | Compound 300 | LCMS m/z 459.26 [M + H]⁺. |
| 319 | 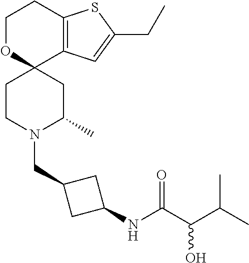 [SMILES: CCc1cc2c(s1)CCO[C@@]21CCN(C[C@H]2C[C@@H](NC(=O)C(O)C(C)C)C2)[C@@H](C)C1] | 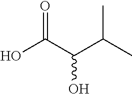 | Compound 300 | LCMS m/z 435.26 [M + H]⁺. |
| 320 | 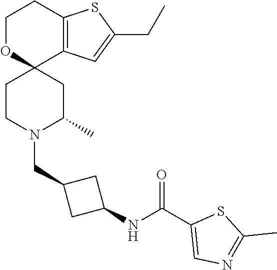 [SMILES: CCc1cc2c(s1)CCO[C@@]21CCN(C[C@H]2C[C@@H](NC(=O)c3cnc(C)s3)C2)[C@@H](C)C1] | 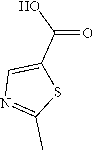 | Compound 300 | LCMS m/z 460.18 [M + H]⁺. |
| 321 | 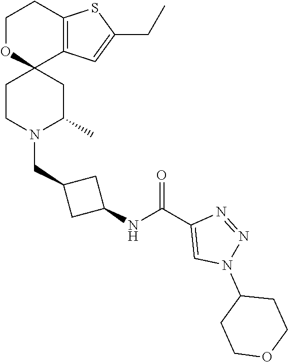 [SMILES: CCc1cc2c(s1)CCO[C@@]21CCN(C[C@H]2C[C@@H](NC(=O)c3cn(C4CCOCC4)nn3)C2)[C@@H](C)C1] | 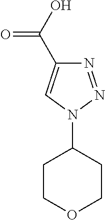 [SMILES: O=C(O)c1cn(C2CCOCC2)nn1] | Compound 300 | LCMS m/z 514.25 [M + H]⁺. |
| 322 | 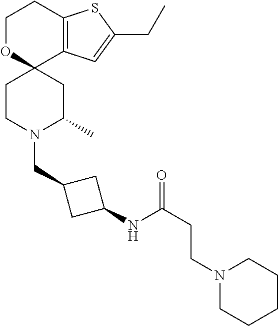 [SMILES: CCc1cc2c(s1)CCO[C@@]21CCN(C[C@H]2C[C@@H](NC(=O)CCN3CCCCC3)C2)[C@@H](C)C1] | 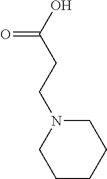 [SMILES: O=C(O)CCN1CCCCC1] | Compound 300 | LCMS m/z 474.28 [M + H]⁺. |
| 323 | 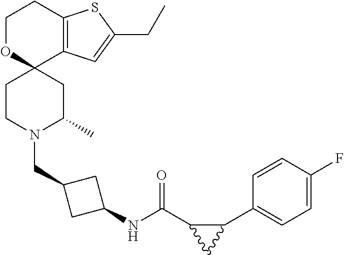 | 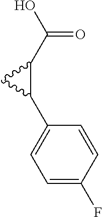 | Compound 300 | LCMS m/z 497.25 [M + H]⁺. |
| 324 | 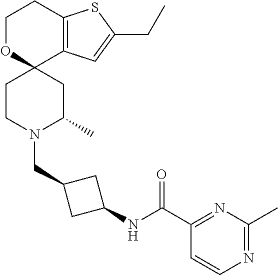 [SMILES: CCc1cc2c(s1)CCO[C@@]21CCN(C[C@H]2C[C@@H](NC(=O)c3ccnc(C)n3)C2)[C@@H](C)C1] | 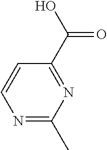 | Compound 300 | LCMS m/z 455.22 [M + H]⁺. |
| 325 | 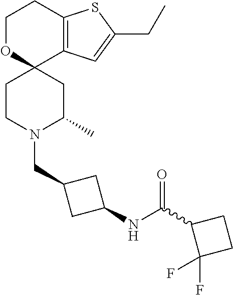 [SMILES: CCc1cc2c(s1)CCO[C@@]21CCN(C[C@H]2C[C@@H](NC(=O)C3CCC3(F)F)C2)[C@@H](C)C1] | 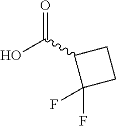 | Compound 300 | LCMS m/z 453.20 [M + H]⁺. |
| 326 | 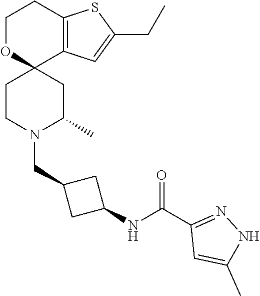 [SMILES: CCc1cc2c(s1)CCO[C@@]21CCN(C[C@H]2C[C@@H](NC(=O)c3cc(C)[nH]n3)C2)[C@@H](C)C1] | 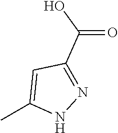 | Compound 300 | LCMS m/z 443.20 [M + H]⁺. |
| 327 | 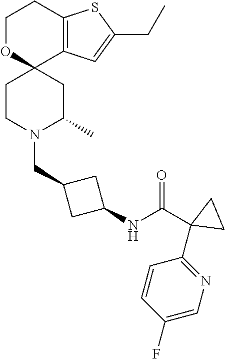 [SMILES: CCc1cc2c(s1)CCO[C@@]21CCN(C[C@H]2C[C@@H](NC(=O)C3(c4ccc(F)cn4)CC3)C2)[C@@H](C)C1] | 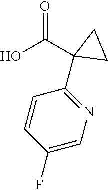 [SMILES: O=C(O)C1(c2ccc(F)cn2)CC1] | Compound 300 | LCMS m/z 498.22 [M + H]⁺. |
| 328 | 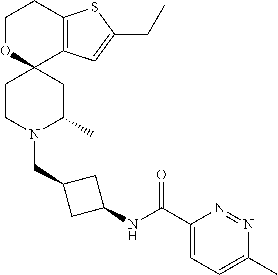 [SMILES: CCc1cc2c(s1)CCO[C@@]21CCN(C[C@H]2C[C@@H](NC(=O)c3ccc(C)nn3)C2)[C@@H](C)C1] | 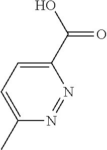 | Compound 300 | LCMS m/z 455.22 [M + H]⁺. |
| 329 | 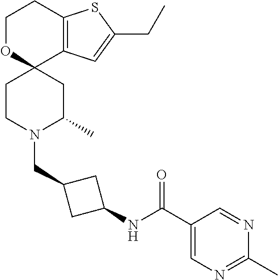 [SMILES: CCc1cc2c(s1)CCO[C@@]21CCN(C[C@H]2C[C@@H](NC(=O)c3cnc(C)nc3)C2)[C@@H](C)C1] | 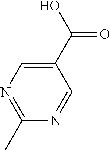 | Compound 300 | LCMS m/z 455.22 [M + H]⁺. |
| 330 | 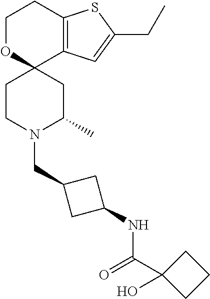 [SMILES: CCc1cc2c(s1)CCO[C@@]21CCN(C[C@H]2C[C@@H](NC(=O)C3(O)CCC3)C2)[C@@H](C)C1] | 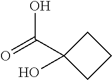 | Compound 300 | LCMS m/z 433.33 [M + H]⁺. |
| 331 | 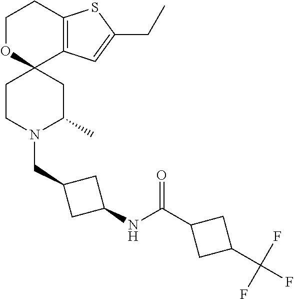 [SMILES: CCc1cc2c(s1)CCO[C@@]21CCN(C[C@H]2C[C@@H](NC(=O)C3CC(C(F)(F)F)C3)C2)[C@@H](C)C1] | 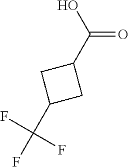 [SMILES: O=C(O)C1CC(C(F)(F)F)C1] | Compound 300 | LCMS m/z 485.39 [M + H]⁺. |
| 332 | 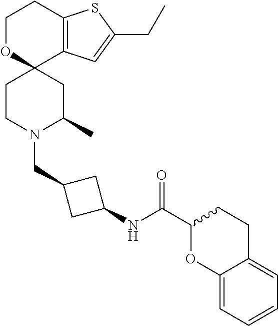 [SMILES: CCc1cc2c(s1)CCO[C@@]21CCN(C[C@H]2C[C@@H](NC(=O)C3CCc4ccccc4O3)C2)[C@H](C)C1] | 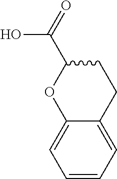 [SMILES: O=C(O)C1CCc2ccccc2O1] | Compound 300 | LCMS m/z 495.23 [M + H]⁺. |
| 333 | 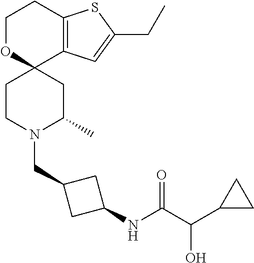 [SMILES: CCc1cc2c(s1)CCO[C@@]21CCN(C[C@H]2C[C@@H](NC(=O)C(O)C3CC3)C2)[C@@H](C)C1] | 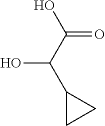 | Compound 300 | LCMS m/z 433.27 [M + H]⁺. |
| 334 | 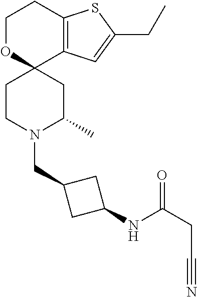 [SMILES: CCc1cc2c(s1)CCO[C@@]21CCN(C[C@H]2C[C@@H](NC(=O)CC#N)C2)[C@@H](C)C1] | 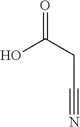 | Compound 300 | LCMS m/z 402.26 [M + H]⁺. |
| 335 | 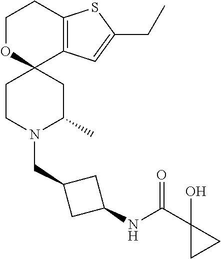 [SMILES: CCc1cc2c(s1)CCO[C@@]21CCN(C[C@H]2C[C@@H](NC(=O)C3(O)CC3)C2)[C@@H](C)C1] | 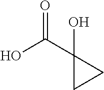 | Compound 300 | LCMS m/z 419.26 [M + H]⁺. |
| 336 | 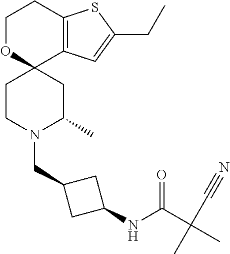 [SMILES: CCc1cc2c(s1)CCO[C@@]21CCN(C[C@H]2C[C@@H](NC(=O)C(C)(C)C#N)C2)[C@@H](C)C1] | 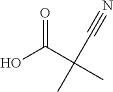 | Compound 300 | LCMS m/z 430.14 [M + H]⁺. |
| 337 | 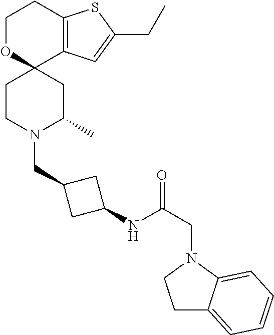 [SMILES: CCc1cc2c(s1)CCO[C@@]21CCN(C[C@H]2C[C@@H](NC(=O)CN3CCc4ccccc43)C2)[C@@H](C)C1] | 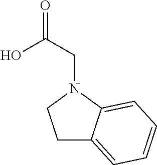 [SMILES: O=C(O)CN1CCc2ccccc21] | Compound 300 | LCMS m/z 494.31 [M + H]⁺. |
| 338 | 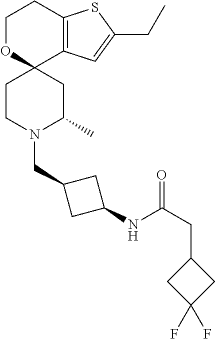 [SMILES: CCc1cc2c(s1)CCO[C@@]21CCN(C[C@H]2C[C@@H](NC(=O)CC3CC(F)(F)C3)C2)[C@@H](C)C1] | 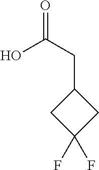 | Compound 300 | LCMS m/z 467.24 [M + H]⁺. |
| 339 | 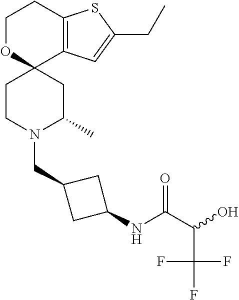 [SMILES: CCc1cc2c(s1)CCO[C@@]21CCN(C[C@H]2C[C@@H](NC(=O)C(O)C(F)(F)F)C2)[C@@H](C)C1] | 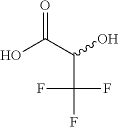 | Compound 300 | LCMS m/z 461.19 [M + H]⁺. |
| 340 | 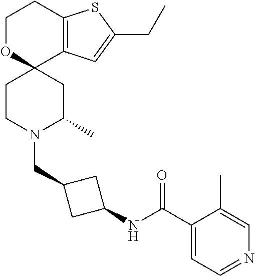 [SMILES: CCc1cc2c(s1)CCO[C@@]21CCN(C[C@H]2C[C@@H](NC(=O)c3ccncc3C)C2)[C@@H](C)C1] | 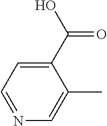 | Compound 300 | LCMS m/z 454.05 [M + H]⁺. |
| 341 | 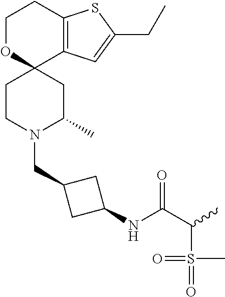 [SMILES: CCc1cc2c(s1)CCO[C@@]21CCN(C[C@H]2C[C@@H](NC(=O)C(C)S(C)(=O)=O)C2)[C@@H](C)C1] | 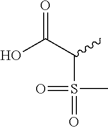 | Compound 300 | LCMS m/z 469.20 [M + H]⁺. |
| 342 | 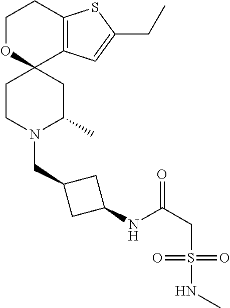 [SMILES: CCc1cc2c(s1)CCO[C@@]21CCN(C[C@H]2C[C@@H](NC(=O)CS(=O)(=O)NC)C2)[C@@H](C)C1] | 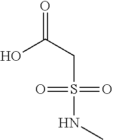 | Compound 300 | LCMS m/z 470.21 [M + H]⁺. |
| 343 | 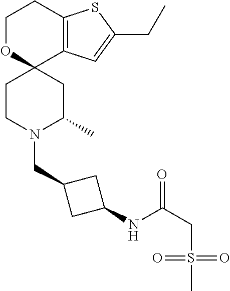 [SMILES: CCc1cc2c(s1)CCO[C@@]21CCN(C[C@H]2C[C@@H](NC(=O)CS(C)(=O)=O)C2)[C@@H](C)C1] | 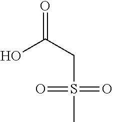 | Compound 300 | LCMS m/z 455.16 [M + H]⁺. |
| 344 | 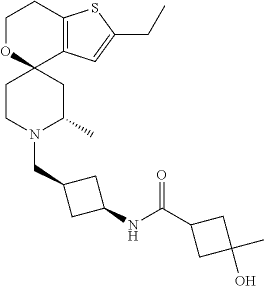 [SMILES: CCc1cc2c(s1)CCO[C@@]21CCN(C[C@H]2C[C@@H](NC(=O)C3CC(C)(O)C3)C2)[C@@H](C)C1] | 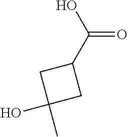 | Compound 300 | LCMS m/z 447.24 [M + H]⁺. |
| 345 | 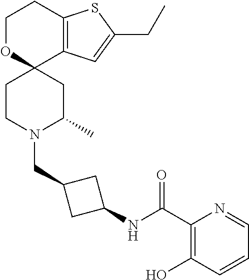 [SMILES: CCc1cc2c(s1)CCO[C@@]21CCN(C[C@H]2C[C@@H](NC(=O)c3ncccc3O)C2)[C@@H](C)C1] | 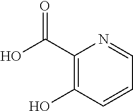 | Compound 300 | LCMS m/z 456.23 [M + H]⁺. |
| 346 | 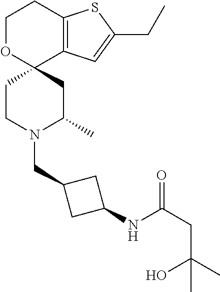 [SMILES: CCc1cc2c(s1)CCO[C@@]21CCN(C[C@H]2C[C@@H](NC(=O)CC(C)(C)O)C2)[C@@H](C)C1] | 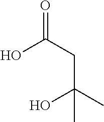 | Compound 300 | LCMS m/z 435.29 [M + H]⁺. |
| 347 | 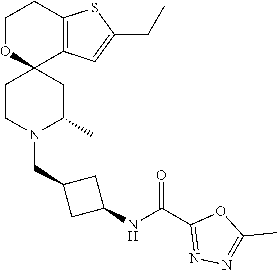 [SMILES: CCc1cc2c(s1)CCO[C@@]21CCN(C[C@H]2C[C@@H](NC(=O)c3nnc(C)o3)C2)[C@@H](C)C1] | 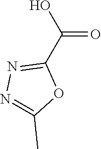 | Compound 300 | LCMS m/z 445.35 [M + H]⁺. |
| 348 | 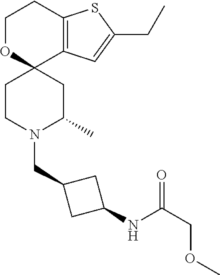 [SMILES: CCc1cc2c(s1)CCO[C@@]21CCN(C[C@H]2C[C@@H](NC(=O)COC)C2)[C@@H](C)C1] | 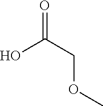 | Compound 300 | LCMS m/z 407.21 [M + H]⁺. |
| 349 | 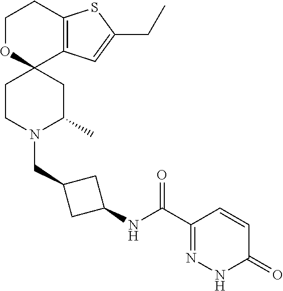 [SMILES: CCc1cc2c(s1)CCO[C@@]21CCN(C[C@H]2C[C@@H](NC(=O)c3ccc(=O)[nH]n3)C2)[C@@H](C)C1] | 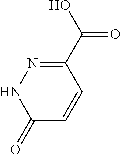 | Compound 300 | LCMS m/z 457.21 [M + H]⁺. |
| 350 | 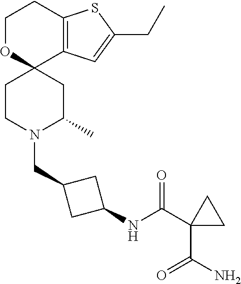 [SMILES: CCc1cc2c(s1)CCO[C@@]21CCN(C[C@H]2C[C@@H](NC(=O)C3(C(N)=O)CC3)C2)[C@@H](C)C1] | 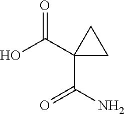 | Compound 300 | LCMS m/z 446.23 [M + H]⁺. |
| 351 | 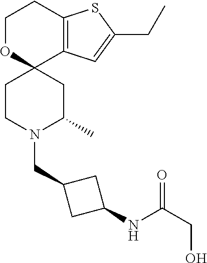 [SMILES: CCc1cc2c(s1)CCO[C@@]21CCN(C[C@H]2C[C@@H](NC(=O)CO)C2)[C@@H](C)C1] | 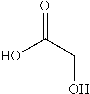 | Compound 300 | LCMS m/z 393.20 [M + H]⁺. |
| 352 | 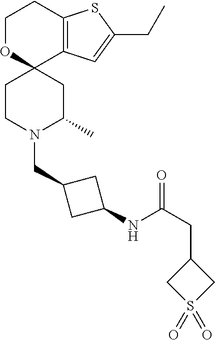 [SMILES: CCc1cc2c(s1)CCO[C@@]21CCN(C[C@H]2C[C@@H](NC(=O)CC3CS(=O)(=O)C3)C2)[C@@H](C)C1] | 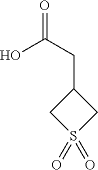 | Compound 300 | LCMS m/z 481.19 [M + H]⁺. |
| 353 | 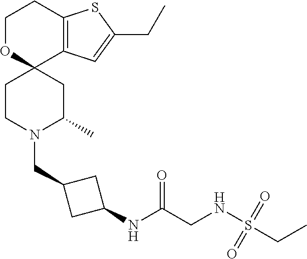 [SMILES: CCc1cc2c(s1)CCO[C@@]21CCN(C[C@H]2C[C@@H](NC(=O)CNS(=O)(=O)CC)C2)[C@@H](C)C1] | 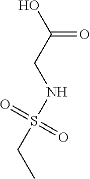 | Compound 300 | LCMS m/z 484.05 [M + H]⁺. |
| 354 | 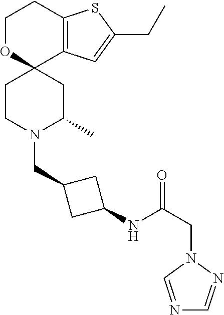 [SMILES: CCc1cc2c(s1)CCO[C@@]21CCN(C[C@H]2C[C@@H](NC(=O)Cn3cncn3)C2)[C@@H](C)C1] | 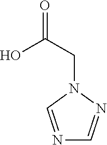 | Compound 300 | LCMS m/z 444.21 [M + H]⁺. |
| 355 | 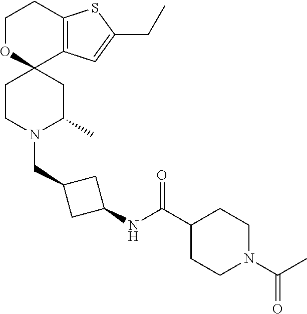 [SMILES: CCc1cc2c(s1)CCO[C@@]21CCN(C[C@H]2C[C@@H](NC(=O)C3CCN(C(C)=O)CC3)C2)[C@@H](C)C1] | 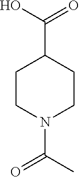 | Compound 300 | LCMS m/z 488.29 [M + H]⁺. |
| 356 | 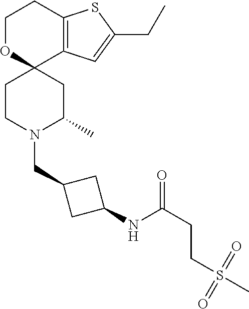 [SMILES: CCc1cc2c(s1)CCO[C@@]21CCN(C[C@H]2C[C@@H](NC(=O)CCS(C)(=O)=O)C2)[C@@H](C)C1] | 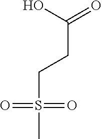 | Compound 300 | LCMS m/z 469.20 [M + H]⁺. |
| 357 | 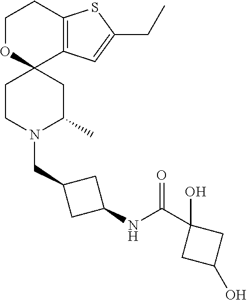 [SMILES: CCc1cc2c(s1)CCO[C@@]21CCN(C[C@H]2C[C@@H](NC(=O)C3(O)CC(O)C3)C2)[C@@H](C)C1] | 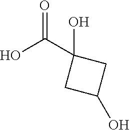 | Compound 300 | LCMS m/z 449.26 [M + H]⁺. |
| 358 | 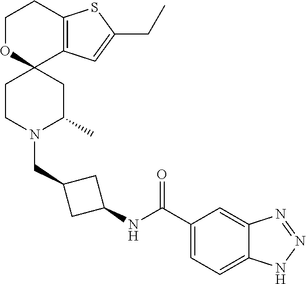 [SMILES: CCc1cc2c(s1)CCO[C@@]21CCN(C[C@H]2C[C@@H](NC(=O)c3ccc4[nH]nnc4c3)C2)[C@@H](C)C1] | 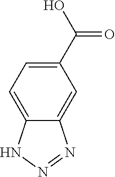 [SMILES: O=C(O)c1ccc2[nH]nnc2c1] | Compound 300 | LCMS m/z 480.21 [M + H]⁺. |
| 359 | 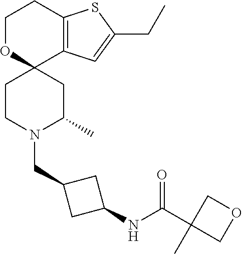 [SMILES: CCc1cc2c(s1)CCO[C@@]21CCN(C[C@H]2C[C@@H](NC(=O)C3(C)COC3)C2)[C@@H](C)C1] | 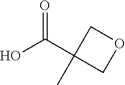 | Compound 300 | LCMS m/z 433.24 [M + H]⁺. |
| 360 | 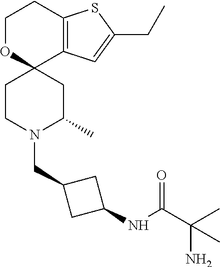 [SMILES: CCc1cc2c(s1)CCO[C@@]21CCN(C[C@H]2C[C@@H](NC(=O)C(C)(C)N)C2)[C@@H](C)C1] | 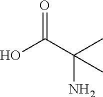 | Compound 300 | LCMS m/z 420.40 [M + H]⁺; |
| 361 | 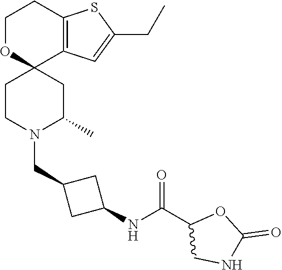 [SMILES: CCc1cc2c(s1)CCO[C@@]21CCN(C[C@H]2C[C@@H](NC(=O)C3CNC(=O)O3)C2)[C@@H](C)C1] | 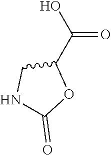 | Compound 300 | LCMS m/z 448.02 [M + H]⁺. |
| 362 | 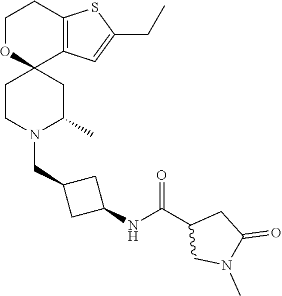 [SMILES: CCc1cc2c(s1)CCO[C@@]21CCN(C[C@H]2C[C@@H](NC(=O)C3CC(=O)N(C)C3)C2)[C@@H](C)C1] | 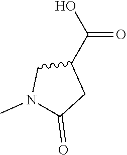 | Compound 300 | LCMS m/z 460.24 [M + H]⁺. |
| 363 | 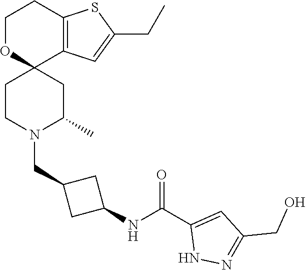 [SMILES: CCc1cc2c(s1)CCO[C@@]21CCN(C[C@H]2C[C@@H](NC(=O)c3cc(CO)n[nH]3)C2)[C@@H](C)C1] | 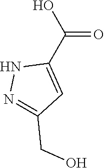 | Compound 300 | LCMS m/z 459.07 [M + H]⁺. |
| 364 | 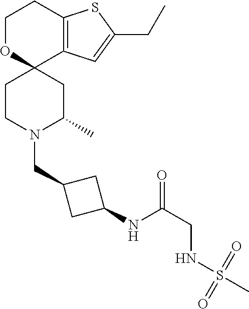 [SMILES: CCc1cc2c(s1)CCO[C@@]21CCN(C[C@H]2C[C@@H](NC(=O)CNS(C)(=O)=O)C2)[C@@H](C)C1] | 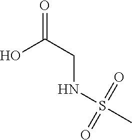 | Compound 300 | LCMS m/z 470.18 [M + H]⁺. |
| 365 | 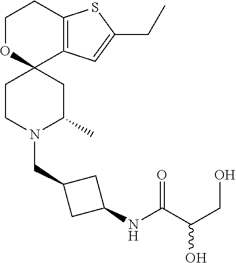 [SMILES: CCc1cc2c(s1)CCO[C@@]21CCN(C[C@H]2C[C@@H](NC(=O)C(O)CO)C2)[C@@H](C)C1] | 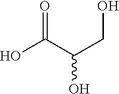 | Compound 300 | LCMS m/z 423.20 [M + H]⁺. |
| 366 | 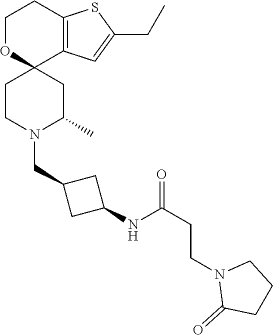 [SMILES: CCc1cc2c(s1)CCO[C@@]21CCN(C[C@H]2C[C@@H](NC(=O)CCN3CCCC3=O)C2)[C@@H](C)C1] | 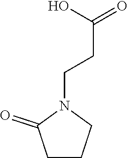 [SMILES: O=C(O)CCN1CCCC1=O] | Compound 300 | LCMS m/z 474.28 [M + H]⁺. |
| 367 | 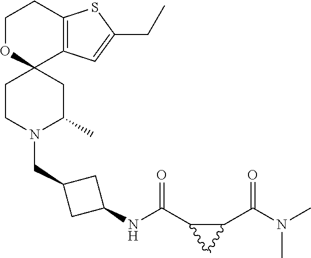 | 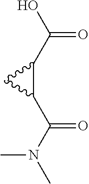 | Compound 300 | LCMS m/z 474.28 [M + H]⁺. |
| 368 | 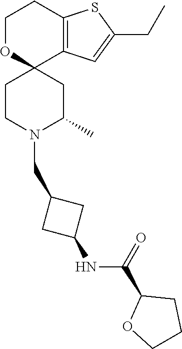 [SMILES: CCc1cc2c(s1)CCO[C@@]21CCN(C[C@H]2C[C@@H](NC(=O)[C@H]3CCCO3)C2)[C@@H](C)C1] | 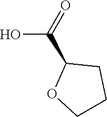 | Compound 301 | LCMS m/z 433.24 [M + H]⁺. |
| 369 | 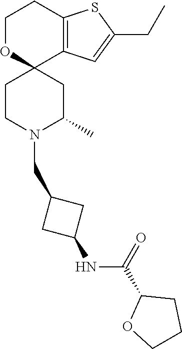 [SMILES: CCc1cc2c(s1)CCO[C@@]21CCN(C[C@H]2C[C@@H](NC(=O)[C@@H]3CCCO3)C2)[C@@H](C)C1] | 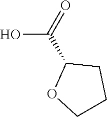 | Compound 301 | LCMS m/z 433.07 [M + H]⁺. |
| 370 | 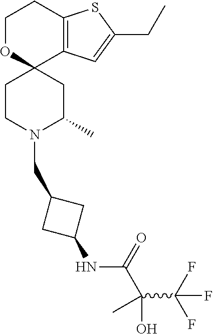 [SMILES: CCc1cc2c(s1)CCO[C@@]21CCN(C[C@H]2C[C@@H](NC(=O)C(C)(O)C(F)(F)F)C2)[C@@H](C)C1] | 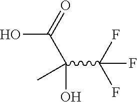 | Compound 301 | LCMS m/z 475.60 [M + H]⁺. |
| 371 | 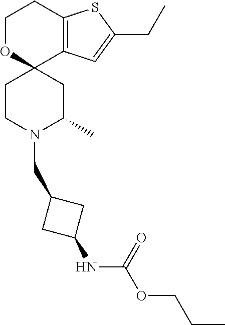 [SMILES: CCCOC(=O)N[C@H]1C[C@@H](CN2CC[C@]3(C[C@@H]2C)OCCc2sc(CC)cc23)C1] | 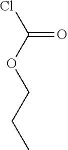 | Compound 302 | LCMS m/z 421.25 [M + H]⁺. |
| 372 | 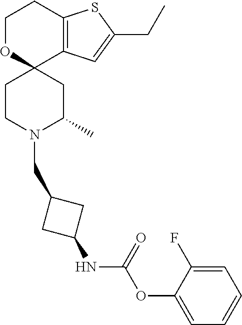 [SMILES: CCc1cc2c(s1)CCO[C@@]21CCN(C[C@H]2C[C@@H](NC(=O)Oc3ccccc3F)C2)[C@@H](C)C1] | 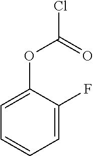 [SMILES: O=C(Cl)Oc1ccccc1F] | Compound 302 | LCMS m/z 473.17 [M + H]⁺. |
| 373 | 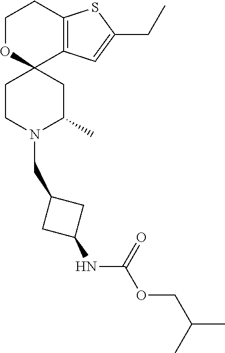 [SMILES: CCc1cc2c(s1)CCO[C@@]21CCN(C[C@H]2C[C@@H](NC(=O)OCC(C)C)C2)[C@@H](C)C1] | 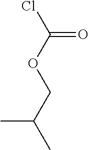 | Compound 302 | LCMS m/z 435.29 [M + H]⁺. |
| 374 | 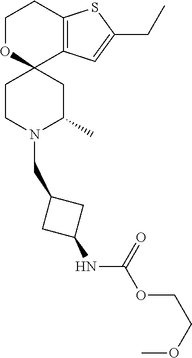 [SMILES: CCc1cc2c(s1)CCO[C@@]21CCN(C[C@H]2C[C@@H](NC(=O)OCCOC)C2)[C@@H](C)C1] | 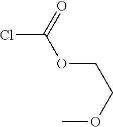 | Compound 302 | LCMS m/z 437.27 [M + H]⁺. |
| 375 | 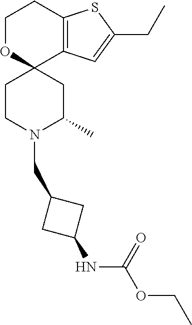 [SMILES: CCOC(=O)N[C@H]1C[C@@H](CN2CC[C@]3(C[C@@H]2C)OCCc2sc(CC)cc23)C1] | 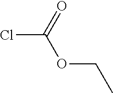 | Compound 302 | LCMS m/z 407.24 [M + H]⁺. |
| 376 | 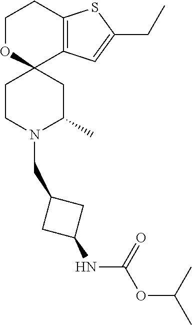 [SMILES: CCc1cc2c(s1)CCO[C@@]21CCN(C[C@H]2C[C@@H](NC(=O)OC(C)C)C2)[C@@H](C)C1] | 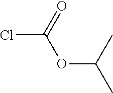 | Compound 302 | LCMS m/z 421.25 [M + H]⁺. |
| 377 | 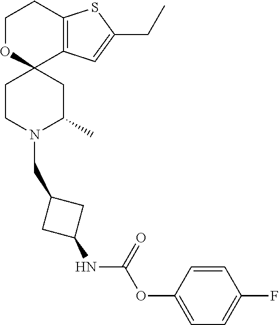 [SMILES: CCc1cc2c(s1)CCO[C@@]21CCN(C[C@H]2C[C@@H](NC(=O)Oc3ccc(F)cc3)C2)[C@@H](C)C1] | 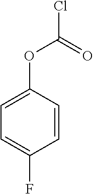 [SMILES: O=C(Cl)Oc1ccc(F)cc1] | Compound 302 | LCMS m/z 473.21 [M + H]⁺. |
| 378 | 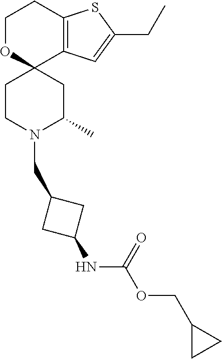 [SMILES: CCc1cc2c(s1)CCO[C@@]21CCN(C[C@H]2C[C@@H](NC(=O)OCC3CC3)C2)[C@@H](C)C1] | 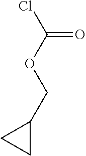 | Compound 302 | LCMS m/z 433.24 [M + H]⁺. |
| 379 | 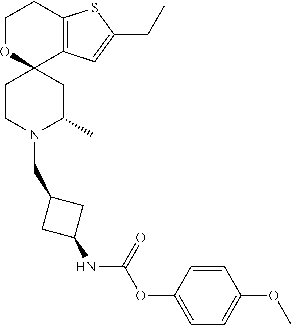 [SMILES: CCc1cc2c(s1)CCO[C@@]21CCN(C[C@H]2C[C@@H](NC(=O)Oc3ccc(OC)cc3)C2)[C@@H](C)C1] | 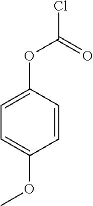 | Compound 302 | LCMS m/z 485.19 [M + H]⁺. |
| 380 | 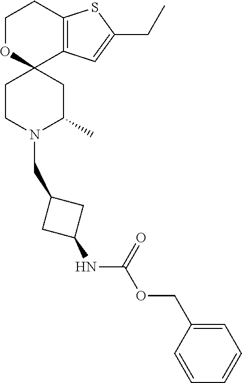 [SMILES: CCc1cc2c(s1)CCO[C@@]21CCN(C[C@H]2C[C@@H](NC(=O)OCc3ccccc3)C2)[C@@H](C)C1] | 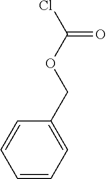 [SMILES: O=C(Cl)OCc1ccccc1] | Compound 302 | LCMS m/z 469.23 [M + H]⁺. |
| 381 | 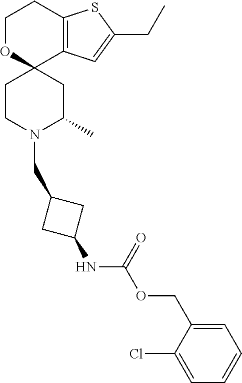 [SMILES: CCc1cc2c(s1)CCO[C@@]21CCN(C[C@H]2C[C@@H](NC(=O)OCc3ccccc3Cl)C2)[C@@H](C)C1] | 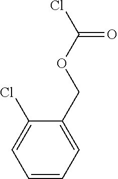 [SMILES: O=C(Cl)OCc1ccccc1Cl] | Compound 302 | LCMS m/z 503.08 [M + H]⁺. |
| 382 | 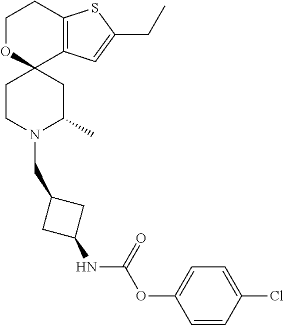 [SMILES: CCc1cc2c(s1)CCO[C@@]21CCN(C[C@H]2C[C@@H](NC(=O)Oc3ccc(Cl)cc3)C2)[C@@H](C)C1] | 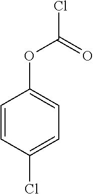 [SMILES: O=C(Cl)Oc1ccc(Cl)cc1] | Compound 302 | LCMS m/z 489.17 [M + H]⁺. |
| 383 | 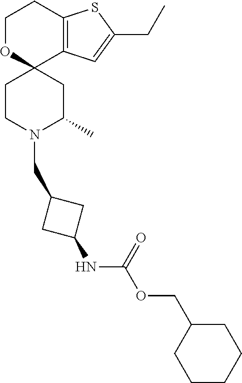 [SMILES: CCc1cc2c(s1)CCO[C@@]21CCN(C[C@H]2C[C@@H](NC(=O)OCC3CCCCC3)C2)[C@@H](C)C1] | 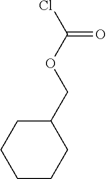 [SMILES: O=C(Cl)OCC1CCCCC1] | Compound 302 | LCMS m/z 475.29 [M + H]⁺. |
| 384 | 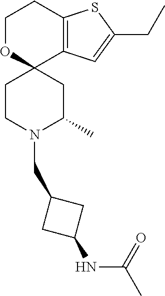 [SMILES: CCc1cc2c(s1)CCO[C@@]21CCN(C[C@H]2C[C@@H](NC(C)=O)C2)[C@@H](C)C1] | 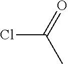 | Compound 302¹ | LCMS m/z 377.07 [M + H]⁺. |

¹DIPEA was used as a base

### Compound 385

### 6-[2-[2-[2-(6-chlorohexoxy)ethoxy]ethoxy]ethoxy]-N-[3-[[(2′S,4R)-2-ethyl-2′-methyl-spiro[6,7-dihydrothieno[3,2-c]pyran-4,4′-piperidine]-1′-yl]methyl]cyclobutyl]hexanamide (385)

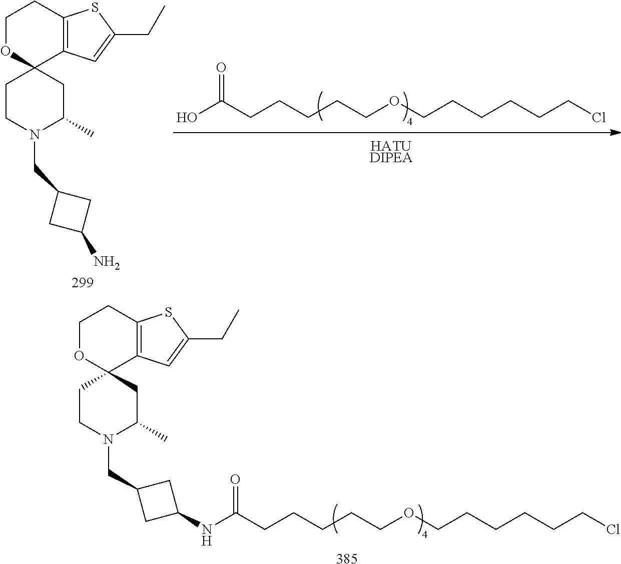

### Preparation of 6-[2-[2-[2-(6-chlorohexoxy)ethoxy]ethoxy]ethoxy]-N-[3-[[(2′S,4R)-2-ethyl-2′-methyl-spiro[6,7-dihydrothieno[3,2-c]pyran-4,4′-piperidine]-1′-yl]methyl]cyclobutyl]hexanamide (385)

3-[[(2′S,4R)-2-ethyl-2′-methyl-spiro[6,7-dihydrothieno[3,2-c]pyran-4,4′-piperidine]-1′-yl]methyl]cyclobutanamine (Trifluoroacetic acid salt) (299) (30 mg, 0.05333 mmol) was dissolved in DMF (1 mL), to which 6-[2-[2-[2-(6-chlorohexoxy)ethoxy]ethoxy]ethoxy]hexanoic acid (25 mg, 0.06529 mmol), DIPEA (50 μL, 0.2871 mmol), and HATU (24 mg, 0.06312 mmol) were added, and the reaction mixture was stirred at room temperature for 1 hour. Purification by reversed-phase chromatography (Column: C18. Gradient: 0-100% MeCN in water with 0.1% TFA) afforded the product 6-[2-[2-[2-(6-chlorohexoxy)ethoxy]ethoxy]ethoxy]-N-[3-[[(2′S,4R)-2-ethyl-2′-methyl-spiro[6,7-dihydrothieno[3,2-c]pyran-4,4′-piperidine]-1′-yl]methyl]cyclobutyl]hexanamide (Trifluoroacetic acid salt) (385) (24 mg, 55%). LCMS m/z 699.24 [M+H]⁺.

### Compound (386)

### 2-[2-[2-[[3-[[(2′S,4R)-2-ethyl-2′-methyl-spiro[6,7-dihydrothieno[3,2-c]pyran-4,4′-piperidine]-1′-yl]methyl]cyclobutyl]carbamoyloxy]ethoxy]ethoxy]ethyl N-[2-[2-(6-chlorohexoxy)ethoxy]ethyl]carbamate (386)

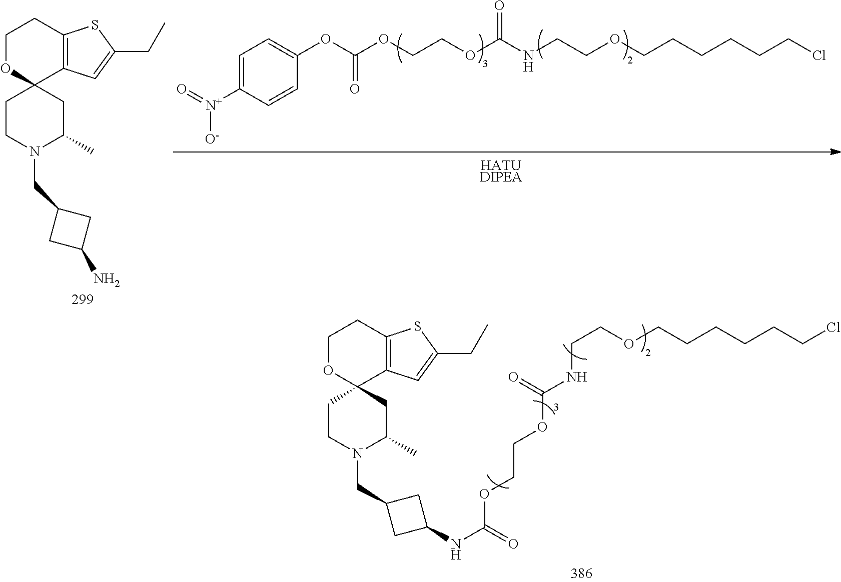

### Preparation of 2-[2-[2-[[3-[[(2′S,4R)-2-ethyl-2′-methyl-spiro[6,7-dihydrothieno[3,2-c]pyran-4,4′-piperidine]-1′-yl]methyl]cyclobutyl]carbamoyloxy]ethoxy]ethoxy]ethyl N-[2-[2-(6-chlorohexoxy)ethoxy]ethyl]carbamate (386)

3-[[(2′S,4R)-2-ethyl-2′-methyl-spiro[6,7-dihydrothieno[3,2-c]pyran-4,4′-piperidine]-1′-yl]methyl]cyclobutanamine (Trifluoroacetic acid salt) (299) (49 mg, 0.08710 mmol) was dissolved in DMF (1 mL), to which 2-[2-[2-[2-[2-(6-chlorohexoxy)ethoxy]ethylcarbamoyloxy]ethoxy]ethoxy]ethyl (4-nitrophenyl) carbonate (59 mg, 0.1044 mmol) and DIPEA (75 μL, 0.4306 mmol) were added, and the reaction was stirred at room temperature for 12 hours. Purification by reversed-phase chromatography (Column: C18. Gradient: 0-100% MeCN in water with 0.1% formic acid) afforded the product 2-[2-[2-[[3-[[(2′S,4R)-2-ethyl-2′-methyl-spiro[6,7-dihydrothieno[3,2-c]pyran-4,4′-piperidine]-1′-yl]methyl]cyclobutyl]carbamoyloxy]ethoxy]ethoxy]ethyl N-[2-[2-(6-chlorohexoxy)ethoxy]ethyl]carbamate (Formic acid salt) (386) (25 mg, 35%). LCMS m/z 760.27 [M+H]⁺.

### Compound 387

### N-[3-[[(2′S,4R)-2-ethyl-2′-methyl-spiro[6,7-dihydrothieno[3,2-c]pyran-4,4′-piperidine]-1′-yl]methyl]cyclobutyl]-2,2,2-trifluoro-acetamide (387)

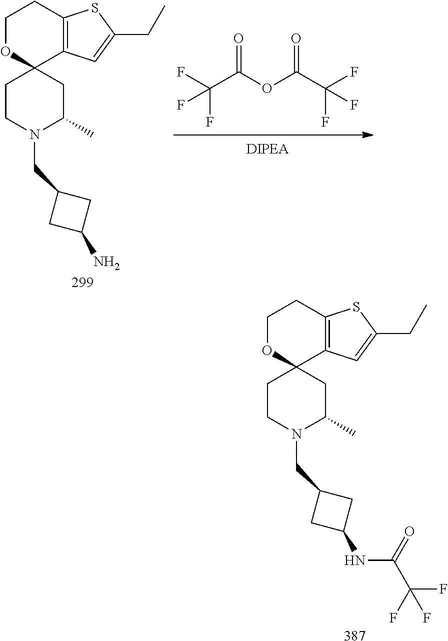

### Preparation of N-[3-[[(2′S,4R)-2-ethyl-2′-methyl-spiro[6,7-dihydrothieno[3,2-c]pyran-4,4′-piperidine]-1′-yl]methyl]cyclobutyl]-2,2,2-trifluoro-acetamide (387)

3-[[(2′S,4R)-2-ethyl-2′-methyl-spiro[6,7-dihydrothieno[3,2-c]pyran-4,4′-piperidine]-1′-yl]methyl]cyclobutanamine (Hydrochloride salt) (299) (20 mg, 0.05391 mmol) was dissolved in DCM (1 mL), to which (2,2,2-trifluoroacetyl) 2,2,2-trifluoroacetate (9 μL, 0.06475 mmol) and DIPEA (19 μL, 0.1091 mmol) were added. The reaction was stirred at room temperature for 2 hours, after which time the solvent was evaporated. Purification by reversed-phase chromatography (Column: C18. Gradient: 0-100% MeCN in water with 0.1% TFA) afforded the product N-[3-[[(2′S,4R)-2-ethyl-2′-methyl-spiro[6,7-dihydrothieno[3,2-c]pyran-4,4′-piperidine]-1′-yl]methyl]cyclobutyl]-2,2,2-trifluoro-acetamide (387) (8.5 mg, 32%). LCMS m/z 431.15 [M+H]⁺.

### Compound 388

### N-[3-[[(2′S,4R)-2-ethyl-2′-methyl-spiro[6,7-dihydrothieno[3,2-c]pyran-4,4′-piperidine]-1′-yl]methyl]cyclobutyl]-2-hydroxy-ethanesulfonamide (388)

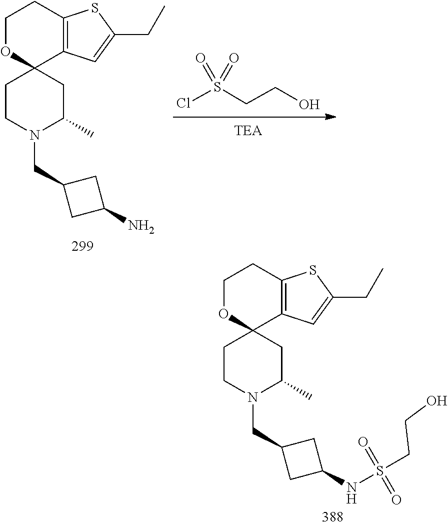 [SMILES: CCc1cc2c(s1)CCO[C@@]21CCN(C[C@H]2C[C@@H](N)C2)[C@@H](C)C1 ; O=S(=O)(Cl)CCO ; CCc1cc2c(s1)CCO[C@@]21CCN(C[C@H]2C[C@@H](NS(=O)(=O)CCO)C2)[C@@H](C)C1]

### Preparation of N-[3-[[(2′S,4R)-2-ethyl-2′-methyl-spiro[6,7-dihydrothieno[3,2-c]pyran-4,4′-piperidine]-1′-yl]methyl]cyclobutyl]-2-hydroxy-ethanesulfonamide (388)

3-[[(2′S,4R)-2-ethyl-2′-methyl-spiro[6,7-dihydrothieno[3,2-c]pyran-4,4′-piperidine]-1′-yl]methyl]cyclobutanamine and 2-hydroxyethanesulfonyl chloride were dissolved in DCM (1 mL), to which TEA (20 μL, 0.1435 mmol) was added. The reaction was stirred at room temperature for 2 hours, after which time the solvent was evaporated, and the crude material was redissolved in minimal MeOH. Purification by reversed-phase HPLC. Method: C18 Waters Sunfire column (30×150 mm, 5 micron). Gradient: MeCN in H₂O with 0.1% trifluoroacetic acid afforded the product N-[3-[[(2′S,4R)-2-ethyl-2′-methyl-spiro[6,7-dihydrothieno[3,2-c]pyran-4,4′-piperidine]-1′-yl]methyl]cyclobutyl]-2-hydroxy-ethanesulfonamide (Trifluoroacetic acid salt) (388) (5.6 mg, 20%). LCMS m/z 443.14 [M+H]⁺.

### Compounds 389-417

Compounds 389-417 (see Table 9) were prepared in a single step from intermediate 299 using coupling method for 388. Sulfonyl chlorides were obtained from commercial sources.

TABLE 9
Structure and physicochemical data for compounds 389-417

| | | | ¹H NMR; LCMS |
|---|---|---|---|
| Cmpd | Structure | Amine Reagent | m/z [M + H]⁺ |
| 389 | 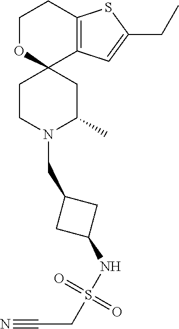 [SMILES: CCc1cc2c(s1)CCO[C@@]21CCN(C[C@H]2C[C@@H](NS(=O)(=O)CC#N)C2)[C@@H](C)C1] | 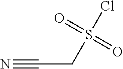 | LCMS m/z 438.15 [M + H]⁺. |
| 390 | 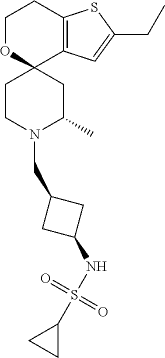 [SMILES: CCc1cc2c(s1)CCO[C@@]21CCN(C[C@H]2C[C@@H](NS(=O)(=O)C3CC3)C2)[C@@H](C)C1] | 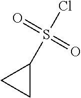 | LCMS m/z 439.16 [M + H]⁺. |
| 391 | 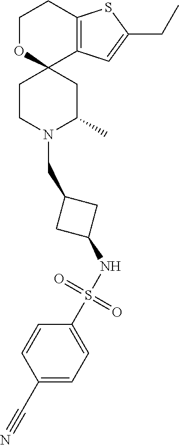 [SMILES: CCc1cc2c(s1)CCO[C@@]21CCN(C[C@H]2C[C@@H](NS(=O)(=O)c3ccc(C#N)cc3)C2)[C@@H](C)C1] | 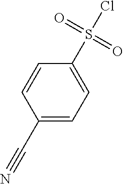 [SMILES: N#Cc1ccc(S(=O)(=O)Cl)cc1] | LCMS m/z 500.01 [M + H]⁺. |
| 392 | 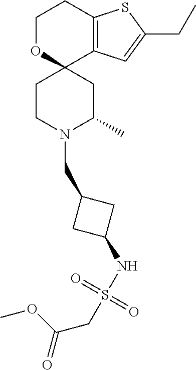 [SMILES: CCc1cc2c(s1)CCO[C@@]21CCN(C[C@H]2C[C@@H](NS(=O)(=O)CC(=O)OC)C2)[C@@H](C)C1] | 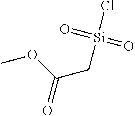 | LCMS m/z 471.15 [M + H]⁺. |
| 393 | 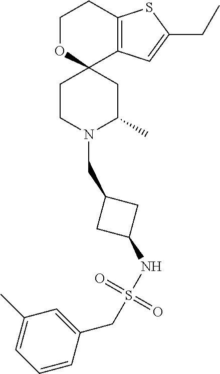 [SMILES: CCc1cc2c(s1)CCO[C@@]21CCN(C[C@H]2C[C@@H](NS(=O)(=O)Cc3cccc(C)c3)C2)[C@@H](C)C1] | 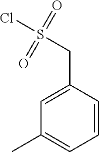 | LCMS m/z 503.37 [M + H]⁺. |
| 394 | 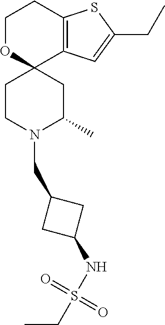 [SMILES: CCc1cc2c(s1)CCO[C@@]21CCN(C[C@H]2C[C@@H](NS(=O)(=O)CC)C2)[C@@H](C)C1] | 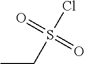 | LCMS m/z 427.21 [M + H]⁺. |
| 395 | 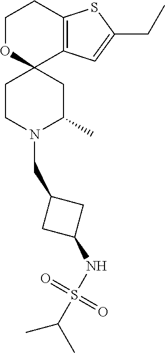 [SMILES: CCc1cc2c(s1)CCO[C@@]21CCN(C[C@H]2C[C@@H](NS(=O)(=O)C(C)C)C2)[C@@H](C)C1] | 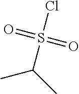 | LCMS m/z 441.28 [M + H]⁺. |
| 396 | 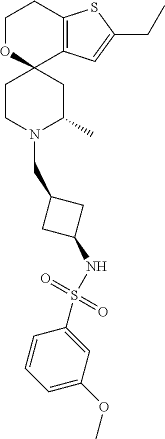 [SMILES: CCc1cc2c(s1)CCO[C@@]21CCN(C[C@H]2C[C@@H](NS(=O)(=O)c3cccc(OC)c3)C2)[C@@H](C)C1] | 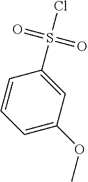 | LCMS m/z 505.19 [M + H]⁺. |
| 397 | 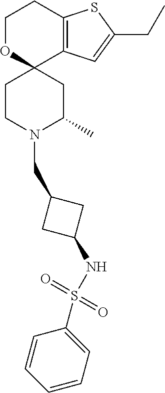 [SMILES: CCc1cc2c(s1)CCO[C@@]21CCN(C[C@H]2C[C@@H](NS(=O)(=O)c3ccccc3)C2)[C@@H](C)C1] | 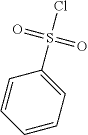 | LCMS m/z 475.19 [M + H]⁺. |
| 398 | 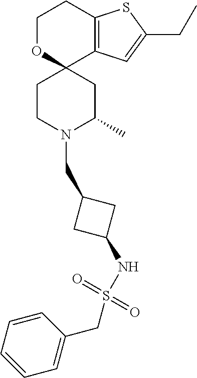 [SMILES: CCc1cc2c(s1)CCO[C@@]21CCN(C[C@H]2C[C@@H](NS(=O)(=O)Cc3ccccc3)C2)[C@@H](C)C1] | 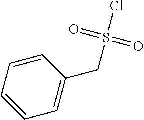 [SMILES: O=S(=O)(Cl)Cc1ccccc1] | LCMS m/z 489.00 [M + H]⁺. |
| 399 | 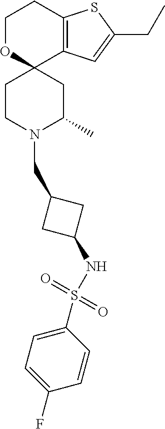 [SMILES: CCc1cc2c(s1)CCO[C@@]21CCN(C[C@H]2C[C@@H](NS(=O)(=O)c3ccc(F)cc3)C2)[C@@H](C)C1] | 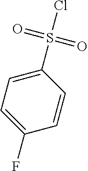 [SMILES: O=S(=O)(Cl)c1ccc(F)cc1] | LCMS m/z 493.17 [M + H]⁺. |
| 400 | 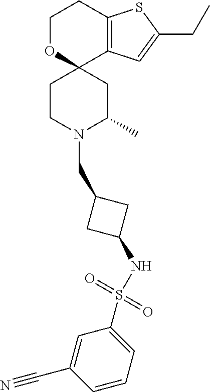 [SMILES: CCc1cc2c(s1)CCO[C@@]21CCN(C[C@H]2C[C@@H](NS(=O)(=O)c3cccc(C#N)c3)C2)[C@@H](C)C1] | 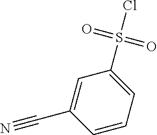 [SMILES: N#Cc1cccc(S(=O)(=O)Cl)c1] | LCMS m/z 500.18 [M + H]⁺. |
| 401 | 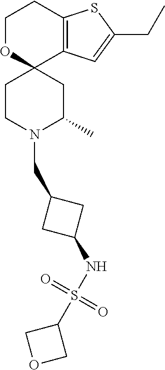 [SMILES: CCc1cc2c(s1)CCO[C@@]21CCN(C[C@H]2C[C@@H](NS(=O)(=O)C3COC3)C2)[C@@H](C)C1] | 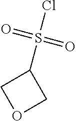 | LCMS m/z 455.19 [M + H]⁺. |
| 402 | 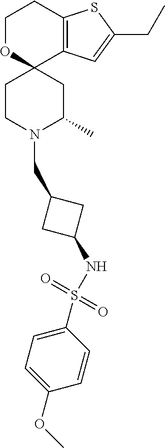 [SMILES: CCc1cc2c(s1)CCO[C@@]21CCN(C[C@H]2C[C@@H](NS(=O)(=O)c3ccc(OC)cc3)C2)[C@@H](C)C1] | 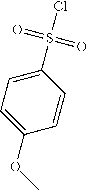 | LCMS m/z 505.03 [M + H]⁺. |
| 403 | 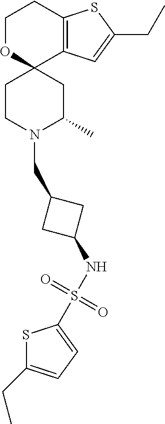 [SMILES: CCc1ccc(S(=O)(=O)N[C@H]2C[C@@H](CN3CC[C@]4(C[C@@H]3C)OCCc3sc(CC)cc34)C2)s1] | 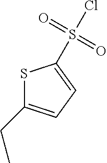 | LCMS m/z 509.13 [M + H]⁺. |
| 404 | 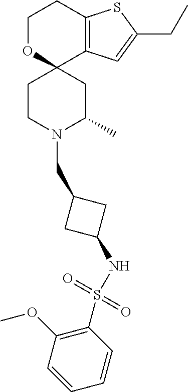 [SMILES: CCc1cc2c(s1)CCO[C@@]21CCN(C[C@H]2C[C@@H](NS(=O)(=O)c3ccccc3OC)C2)[C@@H](C)C1] | 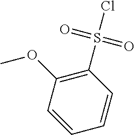 | LCMS m/z 505.39 [M + H]⁺. |
| 405 | 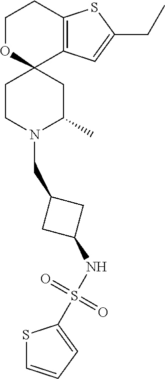 [SMILES: CCc1cc2c(s1)CCO[C@@]21CCN(C[C@H]2C[C@@H](NS(=O)(=O)c3cccs3)C2)[C@@H](C)C1] | 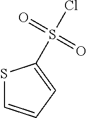 | LCMS m/z 481.15 [M + H]⁺. |
| 406 | 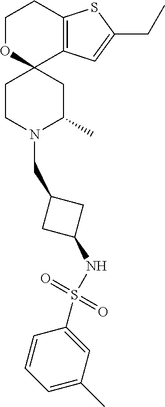 [SMILES: CCc1cc2c(s1)CCO[C@@]21CCN(C[C@H]2C[C@@H](NS(=O)(=O)c3cccc(C)c3)C2)[C@@H](C)C1] | 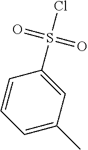 | LCMS m/z 489.17 [M + H]⁺. |
| 407 | 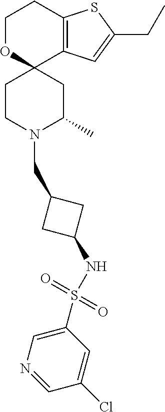 [SMILES: CCc1cc2c(s1)CCO[C@@]21CCN(C[C@H]2C[C@@H](NS(=O)(=O)c3cncc(Cl)c3)C2)[C@@H](C)C1] | 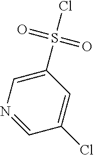 [SMILES: O=S(=O)(Cl)c1cncc(Cl)c1] | LCMS m/z 509.98 [M + H]⁺. |
| 408 | 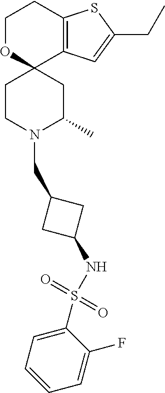 [SMILES: CCc1cc2c(s1)CCO[C@@]21CCN(C[C@H]2C[C@@H](NS(=O)(=O)c3ccccc3F)C2)[C@@H](C)C1] | 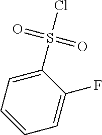 [SMILES: O=S(=O)(Cl)c1ccccc1F] | LCMS m/z 493.14 [M + H]⁺. |
| 409 | 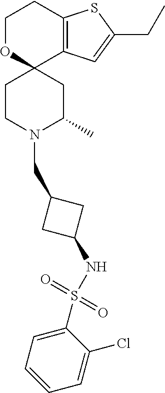 [SMILES: CCc1cc2c(s1)CCO[C@@]21CCN(C[C@H]2C[C@@H](NS(=O)(=O)c3ccccc3Cl)C2)[C@@H](C)C1] | 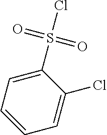 [SMILES: O=S(=O)(Cl)c1ccccc1Cl] | LCMS m/z 509.13 [M + H]⁺. |
| 410 | 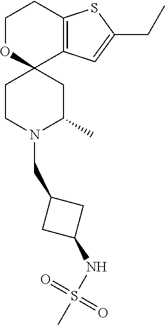 [SMILES: CCc1cc2c(s1)CCO[C@@]21CCN(C[C@H]2C[C@@H](NS(C)(=O)=O)C2)[C@@H](C)C1] | 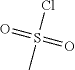 | LCMS m/z 413.20 [M + H]⁺. |
| 411 | 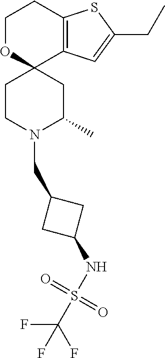 [SMILES: CCc1cc2c(s1)CCO[C@@]21CCN(C[C@H]2C[C@@H](NS(=O)(=O)C(F)(F)F)C2)[C@@H](C)C1] | 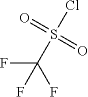 | LCMS m/z 467.21 [M + H]⁺. |
| 412 | 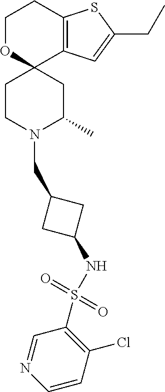 [SMILES: CCc1cc2c(s1)CCO[C@@]21CCN(C[C@H]2C[C@@H](NS(=O)(=O)c3cnccc3Cl)C2)[C@@H](C)C1] | 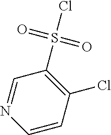 [SMILES: O=S(=O)(Cl)c1cnccc1Cl] | LCMS m/z 510.11 [M + H]⁺. |
| 413 | 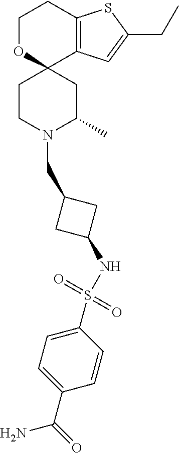 [SMILES: CCc1cc2c(s1)CCO[C@@]21CCN(C[C@H]2C[C@@H](NS(=O)(=O)c3ccc(C(N)=O)cc3)C2)[C@@H](C)C1] | 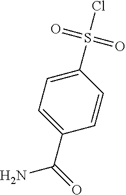 [SMILES: NC(=O)c1ccc(S(=O)(=O)Cl)cc1] | LCMS m/z 518.29 [M + H]⁺. |
| 414 | 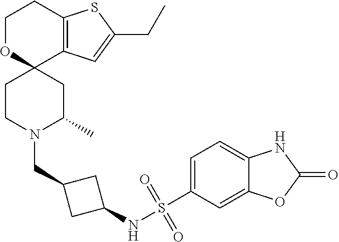 | 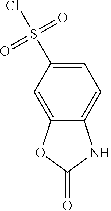 [SMILES: O=c1[nH]c2ccc(S(=O)(=O)Cl)cc2o1] | LCMS m/z 429.07 [M + H]⁺. |
| 415 | 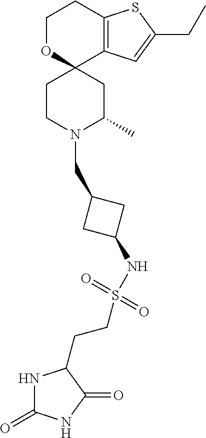 [SMILES: CCc1cc2c(s1)CCO[C@@]21CCN(C[C@H]2C[C@@H](NS(=O)(=O)CCC3NC(=O)NC3=O)C2)[C@@H](C)C1] | 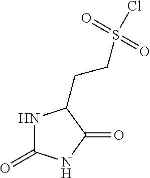 [SMILES: O=C1NC(=O)C(CCS(=O)(=O)Cl)N1] | LCMS m/z 525.39 [M + H]⁺. |
| 416 | 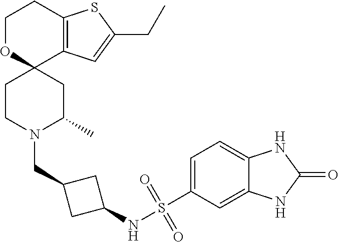 [SMILES: CCc1cc2c(s1)CCO[C@@]21CCN(C[C@H]2C[C@@H](NS(=O)(=O)c3ccc4[nH]c(=O)[nH]c4c3)C2)[C@@H](C)C1] | 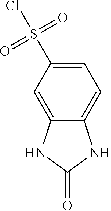 [SMILES: O=c1[nH]c2ccc(S(=O)(=O)Cl)cc2[nH]1] | LCMS m/z 531.06 [M + H]⁺. |
| 417 | 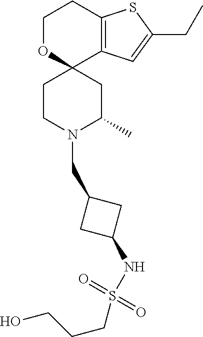 [SMILES: CCc1cc2c(s1)CCO[C@@]21CCN(C[C@H]2C[C@@H](NS(=O)(=O)CCCO)C2)[C@@H](C)C1] | 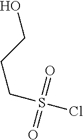 | LCMS m/z 457.28 [M + H]⁺. |

### Preparation S13

### tert-butyl N-(3-formylcyclobutyl)carbamate (S13)

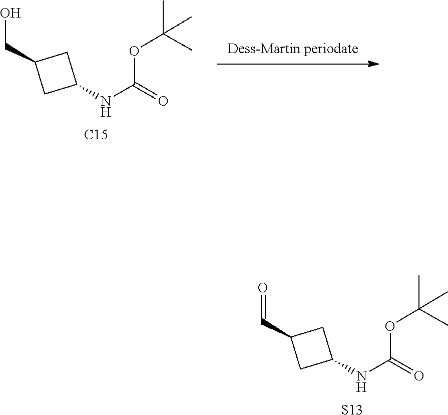

### Preparation of tert-butyl N-(3-formylcyclobutyl)carbamate (S13)

tert-butyl N-[3-(hydroxymethyl)cyclobutyl]carbamate (263 mg, 1.269 mmol) was dissolved in DCM (4 mL), to which Dess Martin periodate (1.211 g, 2.855 mmol) was added. The reaction was stirred at room temperature for 3 hours, after which time it was filtered through a SiO₂ plug with DCM eluent, and evaporated in vacuo. Purification by silica gel chromatography (4 g column, 0-40% EtOAc in Heptanes) afforded the product tert-butyl N-(3-formylcyclobutyl)carbamate (S13) (117 mg, 46%). ¹H NMR (300 MHz, Chloroform-d) δ 9.84 (d, J=1.9 Hz, 1H), 4.75 (s, 1H), 4.25-4.00 (m, 1H), 3.12-2.94 (m, 1H), 2.75-2.60 (m, 2H), 2.28-2.07 (m, 2H), 1.45 (s, 9H). LCMS m/z 200.14 [M+H]⁺.

### Preparation of Compound 419

### 3-[[(2′S,4R)-2-ethyl-2′-methyl-spiro[6,7-dihydrothieno[3,2-c]pyran-4,4′-piperidine]-1′-yl]methyl]cyclobutanamine (419)

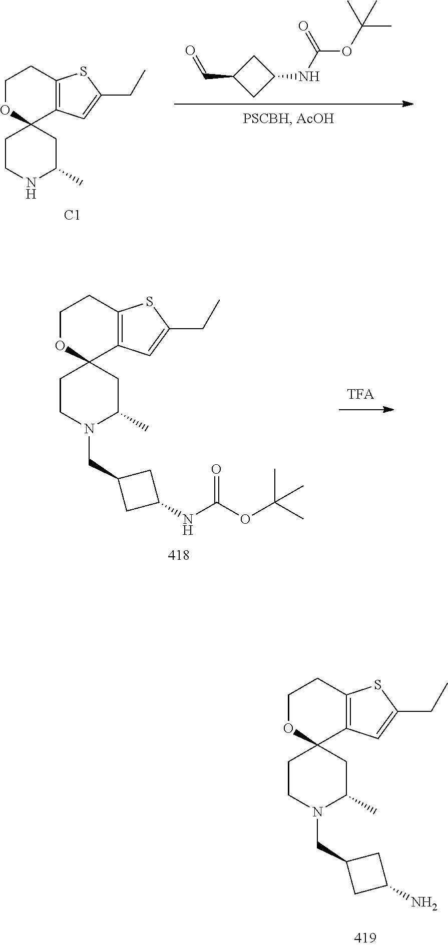

### Step 1. Synthesis of tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (418)

A microwave tube was charged with (2′S,4R)-2-ethyl-2′-methyl-spiro[6,7-dihydrothieno[3,2-c]pyran-4,4′-piperidine]trifluoromethanesulfonate (C1) (116 mg, 0.2887 mmol), tert-butyl N-(3-formylcyclobutyl)carbamate (S13) (110 mg, 0.5571 mmol), polymer-supported cyanoborohydride (455 mg of 2 mmol/g, 0.9104 mmol), and acetic acid (100 μL, 1.758 mmol) in DCM (2 mL). The reaction was heated to 110° C. for 30 minutes under microwave irradiation. After cooling to room temperature, the reaction was filtered, and the filtrate was diluted with DCM (10 mL). The organic phase was washed with 1 M NaOH (10 mL), dried over Na₂SO₄, filtered, and evaporated in vacuo. Purification by silica gel chromatography (4 g column, 0-10% MeOH in DCM) afforded the product tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (418) (19 mg, 15%). ¹H NMR (300 MHz, Chloroform-d) δ 6.49 (s, 1H), 4.91-4.55 (bs, 1H), 4.31-4.03 (m, 1H), 4.01-3.80 (m, 2H), 3.07-2.88 (m, 1H), 2.86-1.59 (m, 17H), 1.46 (s, 9H), 1.28 (t, J=7.5 Hz, 3H), 1.10 (d, J=6.2 Hz, 3H). LCMS m/z 435.40 [M+H]⁺.

### Step 2. Synthesis of 3-[[(2′S,4R)-2-ethyl-2′-methyl-spiro[6,7-dihydrothieno[3,2-c]pyran-4,4′-piperidine]-1′-yl]methyl]cyclobutanamine (Trifluoroacetic Acid (2)) (419)

tert-butyl N-[3-[[(2′S,4R)-2-ethyl-2′-methyl-spiro[6,7-dihydrothieno[3,2-c]pyran-4,4′-piperidine]-1′-yl]methyl]cyclobutyl]carbamate (418) (16 mg, 0.03706 mmol) was dissolved in DCM (300 μL), to which TFA (100 μL, 1.298 mmol) was added. The reaction was stirred at room temperature for 3 hours, and the reaction mixture was then filtered. Purification by reversed-phase HPLC. Method: C18 Waters Sunfire column (30×150 mm, 5 micron). Gradient: MeCN in H₂O with 0.1% trifluoroacetic acid afforded the product 3-[[(2′S,4R)-2-ethyl-2′-methyl-spiro[6,7-dihydrothieno[3,2-c]pyran-4,4′-piperidine]-1′-yl]methyl]cyclobutanamine (Trifluoroacetic Acid (2)) (419) (13 mg, 62%). LCMS m/z 335.21 [M+H]⁺.

### Preparation of Compound 421

### (2′S,4R)-1′-(azetidin-3-ylmethyl)-2-ethyl-2′-methyl-spiro[6,7-dihydrothieno[3,2-c]pyran-4,4′-piperidine](421)

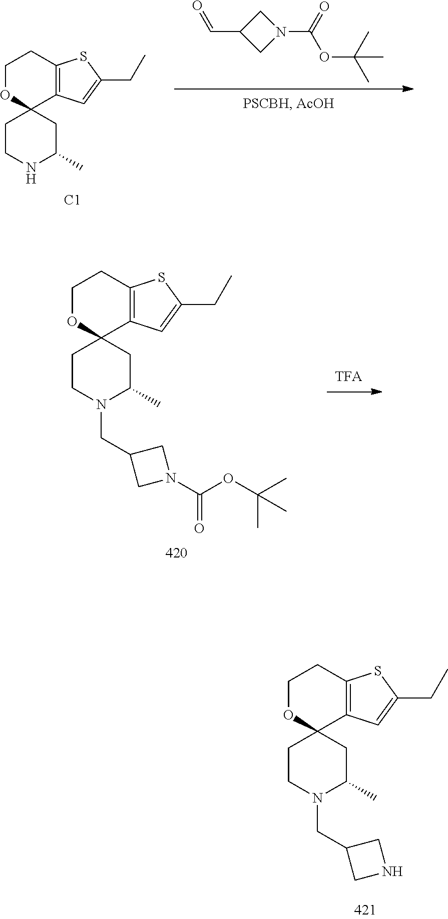

### Step 1. Synthesis of tert-butyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (420)

A microwave tube was charged with (2′S,4R)-2-ethyl-2′-methyl-spiro[6,7-dihydrothieno[3,2-c]pyran-4,4′-piperidine]trifluoromethanesulfonate (C1) (2.5 g, 6.243 mmol), tert-butyl 3-formylazetidine-1-carboxylate (4.65 g, 25.11 mmol), polymer-supported cyanoborohydride (12.5 g of 2 mmol/g, 25.00 mmol), and acetic acid (2.5 mL, 43.96 mmol) in DCM (50 mL). The reaction was heated to 110° C. for 30 minutes under microwave irradiation. After cooling to room temperature, the reaction was filtered, and the filtrate was concentrated in vacuo. Purification by silica gel chromatography (120 g column, 0-5% MeOH in DCM) afforded the product tert-butyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (420) (1.838 g, 67%). ¹H NMR (300 MHz, Chloroform-d) δ 6.48 (s, 1H), 4.05 (td, J=8.4, 4.3 Hz, 2H), 3.98-3.82 (m, 2H), 3.66 (dd, J=8.6, 5.7 Hz, 1H), 3.59 (dd, J=8.5, 5.7 Hz, 1H), 3.07 (dd, J=12.9, 7.8 Hz, 1H), 2.86-2.70 (m, 5H), 2.70-2.53 (m, 2H), 2.53-2.40 (m, 2H), 1.91-1.77 (m, 3H), 1.70-1.58 (m, 1H), 1.46 (s, 9H), 1.28 (t, J=7.5 Hz, 3H), 1.09 (d, J=6.2 Hz, 3H). LCMS m/z 421.5 [M+H]⁺.

### Step 2. Synthesis of (2′S,4R)-1′-(azetidin-3-ylmethyl)-2-ethyl-2′-methyl-spiro[6,7-dihydrothieno[3,2-c]pyran-4,4′-piperidine](421)

TFA (3.0 mL, 38.94 mmol) was added to a solution of tert-butyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (420) (1.8 g, 4.205 mmol) in DCM (12 mL), and the reaction was allowed to stir at room temperature for 3 hours. The resulting solution was concentrated in vacuo to give crude (2′S,4R)-1′-(azetidin-3-ylmethyl)-2-ethyl-2′-methyl-spiro[6,7-dihydrothieno[3,2-c]pyran-4,4′-piperidine](Trifluoroacetic acid (2) salt) (421) (3.657 g, 95%). LCMS m/z 321.22 [M+H]⁺.

### Compound 422

### (4-amino-1,2,5-oxadiazol-3-yl)-[3-[[(2′S,4R)-2-ethyl-2′-methyl-spiro[6,7-dihydrothieno[3,2-c]pyran-4,4′-piperidine]-1′-yl]methyl]azetidin-1-yl]methanone (422)

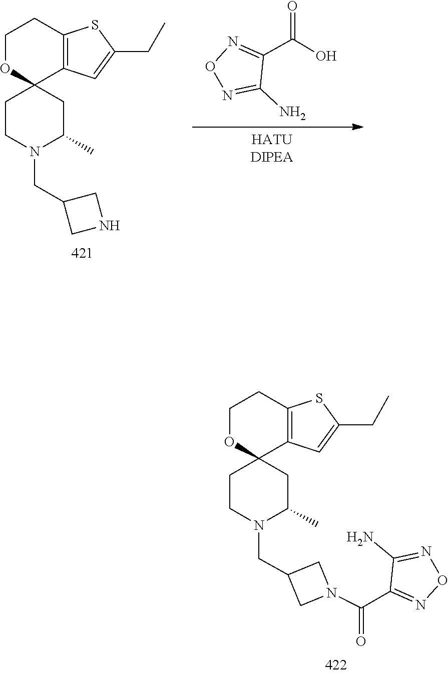 [SMILES: CCc1cc2c(s1)CCO[C@@]21CCN(CC2CNC2)[C@@H](C)C1 ; Nc1nonc1C(=O)O ; CCc1cc2c(s1)CCO[C@@]21CCN(CC2CN(C(=O)c3nonc3N)C2)[C@@H](C)C1]

### Preparation of (4-amino-1,2,5-oxadiazol-3-yl)-[3-[[(2′S,4R)-2-ethyl-2′-methyl-spiro[6,7-dihydrothieno[3,2-c]pyran-4,4′-piperidine]-1′-yl]methyl]azetidin-1-yl]methanone (422)

(2′S,4R)-1′-(azetidin-3-ylmethyl)-2-ethyl-2′-methyl-spiro[6,7-dihydrothieno[3,2-c]pyran-4,4′-piperidine](Trifluoroacetic acid (2) salt) 421 (34 mg, 0.03719 mmol) was dissolved in DMF (300 μL) and DIPEA (50 μL, 0.285 mmol), to which 4-amino-1,2,5-oxadiazole-3-carboxylic acid (16 mg, 0.1228 mmol) and HATU (40 mg, 0.1052 mmol) were added. The reaction was stirred at room temperature for 16 hours, after which time the solution was diluted with MeOH (500 μL) and filtered. Purification by reversed-phase HPLC. Method: C18 Waters Sunfire column (30×150 mm, 5 micron). Gradient: MeCN in H₂O with 5 mM HCl afforded the product (4-amino-1,2,5-oxadiazol-3-yl)-[3-[[(2′S,4R)-2-ethyl-2′-methyl-spiro[6,7-dihydrothieno[3,2-c]pyran-4,4′-piperidine]-1′-yl]methyl]azetidin-1-yl]methanone (Hydrochloride salt) (422) (7.4 mg, 49%). LCMS m/z 432.19 [M+H]⁺.

### Compound 423

### [3-[[(2′S,4R)-2-ethyl-2′-methyl-spiro[6,7-dihydrothieno[3,2-c]pyran-4,4′-piperidine]-1′-yl]methyl]azetidin-1-yl]-(3-hydroxy-2-pyridyl)methanone (423)

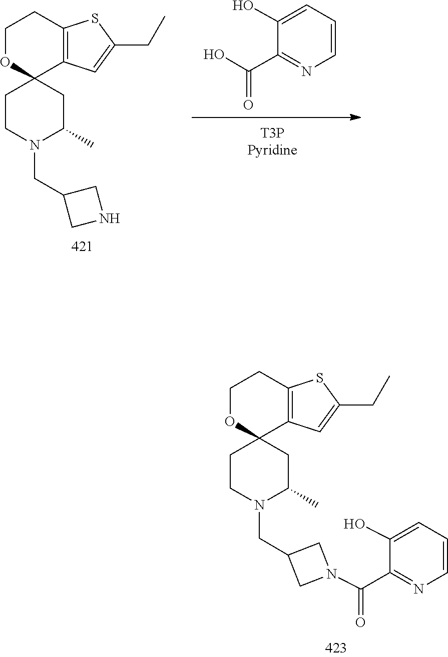 [SMILES: CCc1cc2c(s1)CCO[C@@]21CCN(CC2CNC2)[C@@H](C)C1 ; O=C(O)c1ncccc1O ; CCc1cc2c(s1)CCO[C@@]21CCN(CC2CN(C(=O)c3ncccc3O)C2)[C@@H](C)C1]

### Preparation of [3-[[(2′S,4R)-2-ethyl-2′-methyl-spiro[6,7-dihydrothieno[3,2-c]pyran-4,4′-piperidine]-1′-yl]methyl]azetidin-1-yl]-(3-hydroxy-2-pyridyl)methanone (423)

(2′S,4R)-1′-(azetidin-3-ylmethyl)-2-ethyl-2′-methyl-spiro[6,7-dihydrothieno[3,2-c]pyran-4,4′-piperidine]421 (20 mg, 0.0624 mmol) was dissolved in DMF (2 mL) and pyridine (50 μL, 0.6182 mmol), to which T3P (40 mg, 0.1257 mmol) and 3-hydroxypyridine-2-carboxylic acid (25 mg, 0.1797 mmol) were added. The reaction was allowed to stir at room temperature overnight. Purification by reversed-phase HPLC. Method: C18 Waters Sunfire column (30×150 mm, 5 micron). Gradient: MeCN in H₂O with 0.1% trifluoroacetic acid afforded [3-[[(2′S,4R)-2-ethyl-2′-methyl-spiro[6,7-dihydrothieno[3,2-c]pyran-4,4′-piperidine]-1′-yl]methyl]azetidin-1-yl]-(3-hydroxy-2-pyridyl)methanone (2.8 mg, 10%) (423). LCMS m/z 442.16 [M+H]⁺.

### Compound 424

### methyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (424)

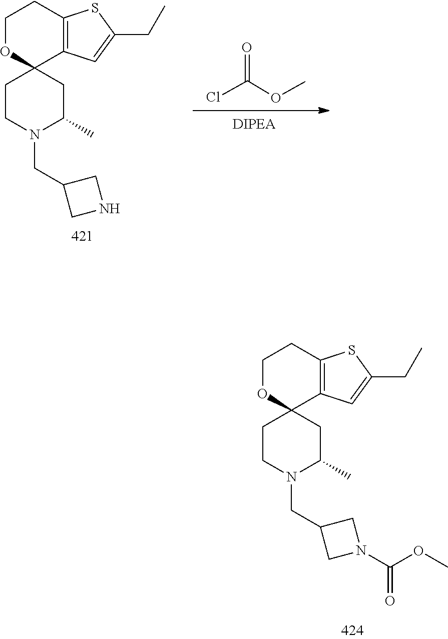

### Preparation of methyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (424)

(2′S,4R)-1′-(azetidin-3-ylmethyl)-2-ethyl-2′-methyl-spiro[6,7-dihydrothieno[3,2-c]pyran-4,4′-piperidine](Trifluoroacetic acid (2) salt) 421 (34 mg, 0.03719 mmol) was dissolved in DMF (300 μL) and DIPEA (50 μL, 0.285 mmol), to which methyl carbonochloridate (9.5 μL, 0.1228 mmol) was added. The reaction was stirred at room temperature for 10 minutes, after which time it was filtered. Purification by reversed-phase HPLC. Method: C18 Waters Sunfire column (30×150 mm, 5 micron). Gradient: MeCN in H2O with 5 mM HCl afforded the product methyl 3-[[(2′S,4R)-2-ethyl-2′-methyl-spiro[6,7-dihydrothieno[3,2-c]pyran-4,4′-piperidine]-1′-yl]methyl]azetidine-1-carboxylate (Hydrochloride salt) (424) (13.4 mg, 86%). LCMS m/z 379.22 [M+H]⁺.

### Compounds 425-490

Compounds 425-490 (see Table 10) were prepared from intermediate 421 using the appropriate reagent and using the amide formation methods as described for compounds 422-424. Coupling partners were obtained from commercial sources.

TABLE 10
Method of preparation, structure and physicochemical data for compounds 425-490

| | | Amide | | |
|---|---|---|---|---|
| | | Coupling | | ¹H NMR; LCMS |
| Cmpd | Structure | Partner | Method | m/z [M + H]⁺ |
| 425 | 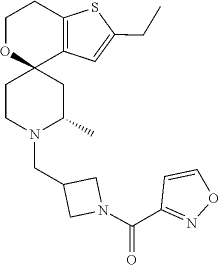 [SMILES: CCc1cc2c(s1)CCO[C@@]21CCN(CC2CN(C(=O)c3ccon3)C2)[C@@H](C)C1] | 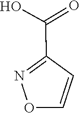 | 422 | LCMS m/z 416.2 [M + H]⁺. |
| 426 | 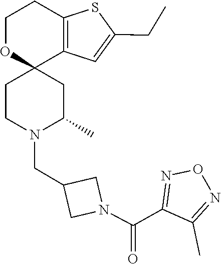 [SMILES: CCc1cc2c(s1)CCO[C@@]21CCN(CC2CN(C(=O)c3nonc3C)C2)[C@@H](C)C1] | 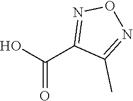 | 422 | LCMS m/z 431.22 [M + H]⁺. |
| 427 | 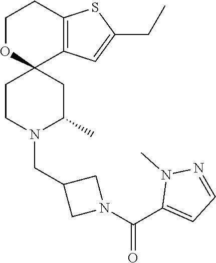 [SMILES: CCc1cc2c(s1)CCO[C@@]21CCN(CC2CN(C(=O)c3ccnn3C)C2)[C@@H](C)C1] | 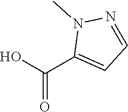 | 422 | LCMS m/z 429.23 [M + H]⁺. |
| 428 | 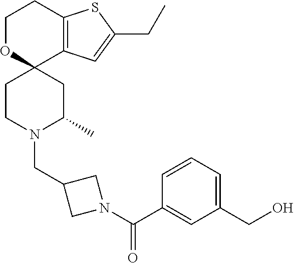 [SMILES: CCc1cc2c(s1)CCO[C@@]21CCN(CC2CN(C(=O)c3cccc(CO)c3)C2)[C@@H](C)C1] | 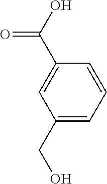 [SMILES: O=C(O)c1cccc(CO)c1] | 422 | LCMS m/z 455.22 [M + H]⁺. |
| 429 | 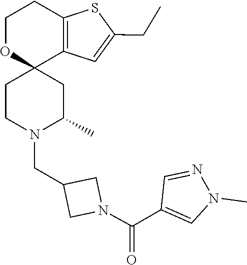 [SMILES: CCc1cc2c(s1)CCO[C@@]21CCN(CC2CN(C(=O)c3cnn(C)c3)C2)[C@@H](C)C1] | 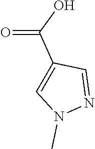 | 422 | LCMS m/z 429.23 [M + H]⁺. |
| 430 | 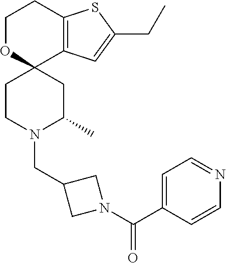 [SMILES: CCc1cc2c(s1)CCO[C@@]21CCN(CC2CN(C(=O)c3ccncc3)C2)[C@@H](C)C1] | 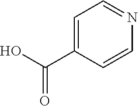 | 422 | LCMS m/z 426.23 [M + H]⁺. |
| 431 | 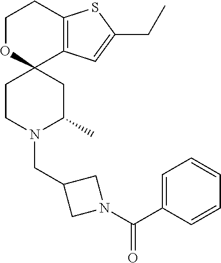 [SMILES: CCc1cc2c(s1)CCO[C@@]21CCN(CC2CN(C(=O)c3ccccc3)C2)[C@@H](C)C1] | 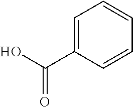 | 422 | LCMS m/z 425.35 [M + H]⁺. |
| 432 | 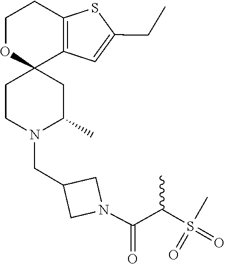 [SMILES: CCc1cc2c(s1)CCO[C@@]21CCN(CC2CN(C(=O)C(C)S(C)(=O)=O)C2)[C@@H](C)C1] | 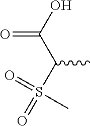 | 422 | LCMS m/z 455.19 [M + H]⁺. |
| 433 | 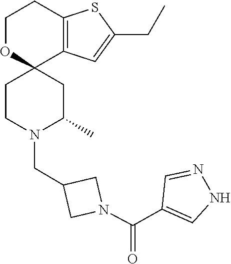 [SMILES: CCc1cc2c(s1)CCO[C@@]21CCN(CC2CN(C(=O)c3cn[nH]c3)C2)[C@@H](C)C1] | 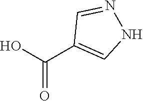 | 422 | LCMS m/z 415.22 [M + H]⁺. |
| 434 | 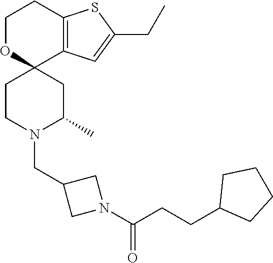 [SMILES: CCc1cc2c(s1)CCO[C@@]21CCN(CC2CN(C(=O)CCC3CCCC3)C2)[C@@H](C)C1] | 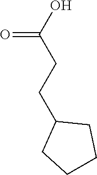 | 422 | LCMS m/z 445.13 [M + H]⁺. |
| 435 | 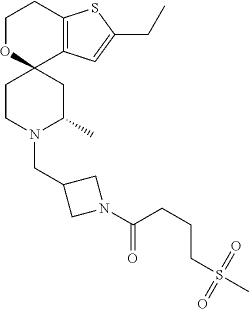 [SMILES: CCc1cc2c(s1)CCO[C@@]21CCN(CC2CN(C(=O)CCCS(C)(=O)=O)C2)[C@@H](C)C1] | 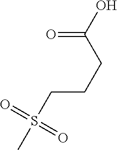 | 422 | LCMS m/z 469.39 [M + H]⁺. |
| 436 | 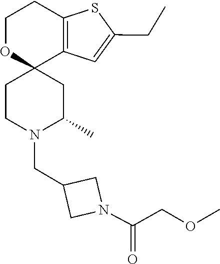 [SMILES: CCc1cc2c(s1)CCO[C@@]21CCN(CC2CN(C(=O)COC)C2)[C@@H](C)C1] | 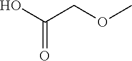 | 422 | LCMS m/z 393.23 [M + H]⁺. |
| 437 | 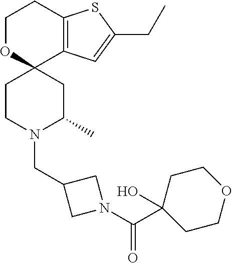 [SMILES: CCc1cc2c(s1)CCO[C@@]21CCN(CC2CN(C(=O)C3(O)CCOCC3)C2)[C@@H](C)C1] | 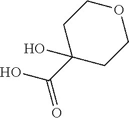 | 422 | LCMS m/z 449.23 [M + H]⁺. |
| 438 | 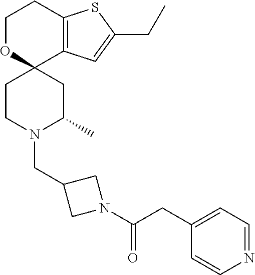 [SMILES: CCc1cc2c(s1)CCO[C@@]21CCN(CC2CN(C(=O)Cc3ccncc3)C2)[C@@H](C)C1] | 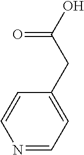 | 422 | LCMS m/z 440.21 [M + H]⁺. |
| 439 | 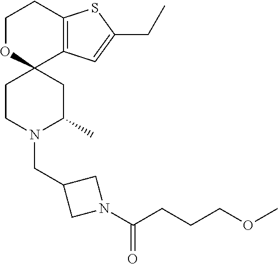 [SMILES: CCc1cc2c(s1)CCO[C@@]21CCN(CC2CN(C(=O)CCCOC)C2)[C@@H](C)C1] | 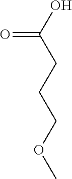 | 422 | LCMS m/z 421.28 [M + H]⁺. |
| 440 | 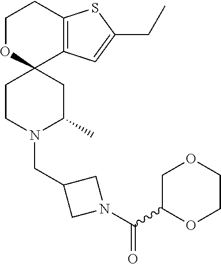 [SMILES: CCc1cc2c(s1)CCO[C@@]21CCN(CC2CN(C(=O)C3COCCO3)C2)[C@@H](C)C1] | 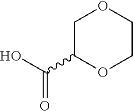 | 422 | LCMS m/z 435.26 [M + H]⁺. |
| 441 | 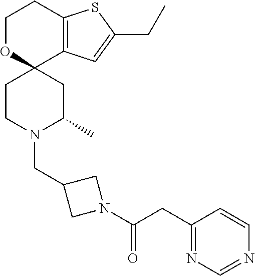 [SMILES: CCc1cc2c(s1)CCO[C@@]21CCN(CC2CN(C(=O)Cc3ccncn3)C2)[C@@H](C)C1] | 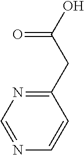 | 422 | LCMS m/z 441.25 [M + H]⁺. |
| 442 | 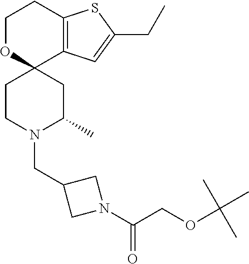 [SMILES: CCc1cc2c(s1)CCO[C@@]21CCN(CC2CN(C(=O)COC(C)(C)C)C2)[C@@H](C)C1] | 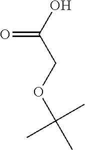 | 422 | LCMS m/z 435.42 [M + H]⁺. |
| 443 | 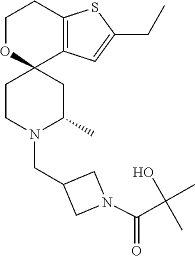 [SMILES: CCc1cc2c(s1)CCO[C@@]21CCN(CC2CN(C(=O)C(C)(C)O)C2)[C@@H](C)C1] | 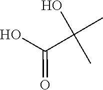 | 422 | LCMS m/z 407.27 [M + H]⁺. |
| 444 | 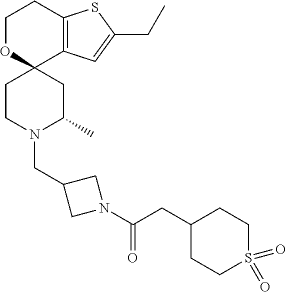 [SMILES: CCc1cc2c(s1)CCO[C@@]21CCN(CC2CN(C(=O)CC3CCS(=O)(=O)CC3)C2)[C@@H](C)C1] | 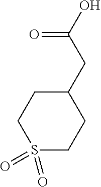 [SMILES: O=C(O)CC1CCS(=O)(=O)CC1] | 422 | LCMS m/z 495.23 [M + H]⁺. |
| 445 | 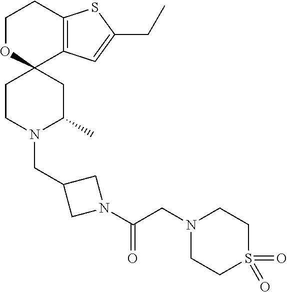 [SMILES: CCc1cc2c(s1)CCO[C@@]21CCN(CC2CN(C(=O)CN3CCS(=O)(=O)CC3)C2)[C@@H](C)C1] | 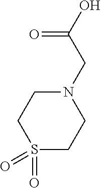 [SMILES: O=C(O)CN1CCS(=O)(=O)CC1] | 422 | LCMS m/z 496.33 [M + H]⁺. |
| 446 | 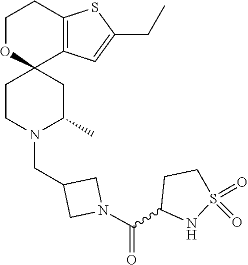 [SMILES: CCc1cc2c(s1)CCO[C@@]21CCN(CC2CN(C(=O)C3CCS(=O)(=O)N3)C2)[C@@H](C)C1] | 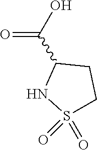 | 422 | LCMS m/z 468.19 [M + H]⁺. |
| 447 | 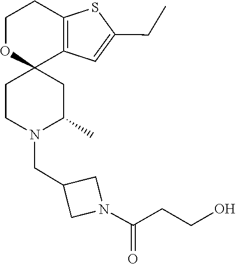 [SMILES: CCc1cc2c(s1)CCO[C@@]21CCN(CC2CN(C(=O)CCO)C2)[C@@H](C)C1] | 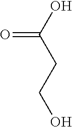 | 422 | LCMS m/z 393.23 [M + H]⁺. |
| 448 | 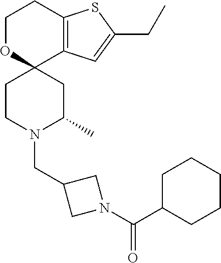 [SMILES: CCc1cc2c(s1)CCO[C@@]21CCN(CC2CN(C(=O)C3CCCCC3)C2)[C@@H](C)C1] | 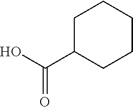 | 422 | LCMS m/z 431.25 [M + H]⁺. |
| 449 | 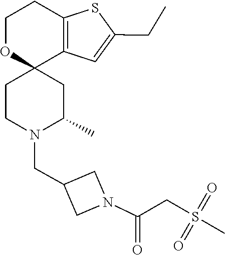 [SMILES: CCc1cc2c(s1)CCO[C@@]21CCN(CC2CN(C(=O)CS(C)(=O)=O)C2)[C@@H](C)C1] | 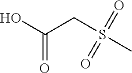 | 422 | LCMS m/z 441.18 [M + H]⁺. |
| 450 | 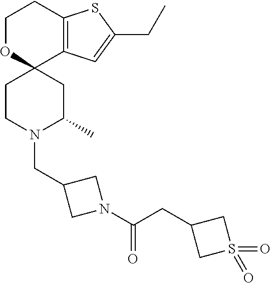 [SMILES: CCc1cc2c(s1)CCO[C@@]21CCN(CC2CN(C(=O)CC3CS(=O)(=O)C3)C2)[C@@H](C)C1] | 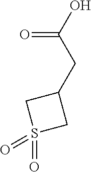 | 422 | LCMS m/z 467.18 [M + H]⁺. |
| 451 | 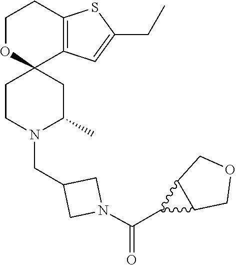 | 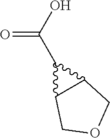 | 422 | LCMS m/z 431.22 [M + H]⁺. |
| 452 | 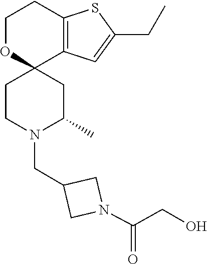 [SMILES: CCc1cc2c(s1)CCO[C@@]21CCN(CC2CN(C(=O)CO)C2)[C@@H](C)C1] | 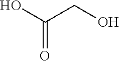 | 422 | LCMS m/z 379.22 [M + H]⁺. |
| 453 | 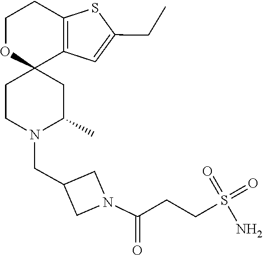 [SMILES: CCc1cc2c(s1)CCO[C@@]21CCN(CC2CN(C(=O)CCS(N)(=O)=O)C2)[C@@H](C)C1] | 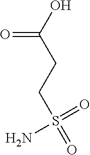 | 422 | LCMS m/z 456.20 [M + H]⁺. |
| 454 | 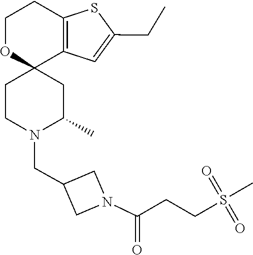 [SMILES: CCc1cc2c(s1)CCO[C@@]21CCN(CC2CN(C(=O)CCS(C)(=O)=O)C2)[C@@H](C)C1] | 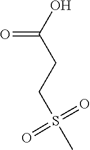 | 422 | LCMS m/z 455.22 [M + H]⁺. |
| 455 | 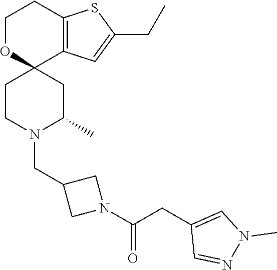 [SMILES: CCc1cc2c(s1)CCO[C@@]21CCN(CC2CN(C(=O)Cc3cnn(C)c3)C2)[C@@H](C)C1] | 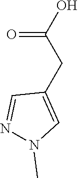 | 422 | LCMS m/z 443.37 [M + H]⁺. |
| 456 | 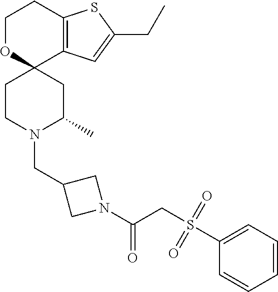 [SMILES: CCc1cc2c(s1)CCO[C@@]21CCN(CC2CN(C(=O)CS(=O)(=O)c3ccccc3)C2)[C@@H](C)C1] | 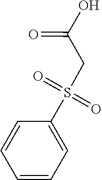 [SMILES: O=C(O)CS(=O)(=O)c1ccccc1] | 422 | LCMS m/z 503.08 [M + H]⁺. |
| 457 | 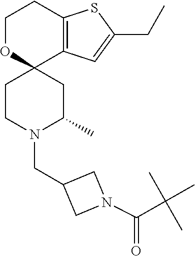 [SMILES: CCc1cc2c(s1)CCO[C@@]21CCN(CC2CN(C(=O)C(C)(C)C)C2)[C@@H](C)C1] | 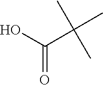 | 422 | LCMS m/z 405.25 [M + H]⁺. |
| 458 | 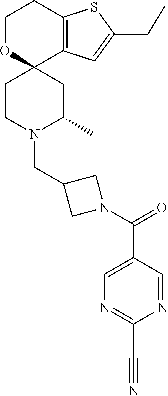 [SMILES: CCc1cc2c(s1)CCO[C@@]21CCN(CC2CN(C(=O)c3cnc(C#N)nc3)C2)[C@@H](C)C1] | 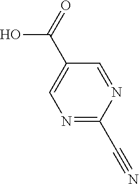 [SMILES: N#Cc1ncc(C(=O)O)cn1] | 423 | LCMS m/z 452.19 [M + H]⁺. |
| 459 | 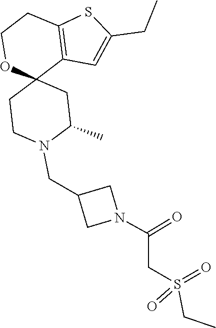 [SMILES: CCc1cc2c(s1)CCO[C@@]21CCN(CC2CN(C(=O)CS(=O)(=O)CC)C2)[C@@H](C)C1] | 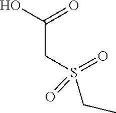 | 423 | LCMS m/z 455.00 [M + H]⁺. |
| 460 | 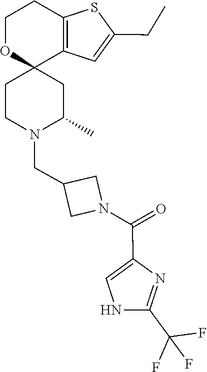 [SMILES: CCc1cc2c(s1)CCO[C@@]21CCN(CC2CN(C(=O)c3c[nH]c(C(F)(F)F)n3)C2)[C@@H](C)C1] | 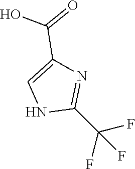 [SMILES: O=C(O)c1c[nH]c(C(F)(F)F)n1] | 423 | LCMS m/z 483.21 [M + H]⁺. |
| 461 | 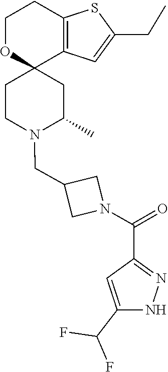 [SMILES: CCc1cc2c(s1)CCO[C@@]21CCN(CC2CN(C(=O)c3cc(C(F)F)[nH]n3)C2)[C@@H](C)C1] | 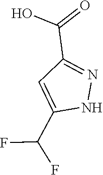 [SMILES: O=C(O)c1cc(C(F)F)[nH]n1] | 423 | LCMS m/z 465.35 [M + H]⁺. |
| 462 | 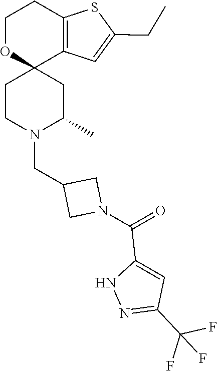 [SMILES: CCc1cc2c(s1)CCO[C@@]21CCN(CC2CN(C(=O)c3cc(C(F)(F)F)n[nH]3)C2)[C@@H](C)C1] | 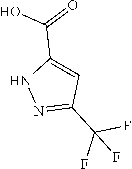 [SMILES: O=C(O)c1cc(C(F)(F)F)n[nH]1] | 423 | LCMS m/z 483.04 [M + H]⁺. |
| 463 | 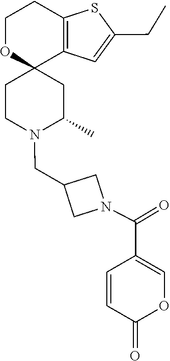 [SMILES: CCc1cc2c(s1)CCO[C@@]21CCN(CC2CN(C(=O)c3ccc(=O)oc3)C2)[C@@H](C)C1] | 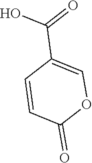 | 423 | LCMS m/z 443.30 [M + H]⁺. |
| 464 | 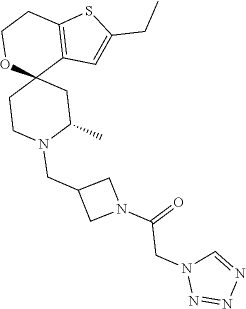 [SMILES: CCc1cc2c(s1)CCO[C@@]21CCN(CC2CN(C(=O)Cn3cnnn3)C2)[C@@H](C)C1] | 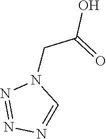 | 423 | LCMS m/z 431.31 [M + H]⁺. |
| 465 | 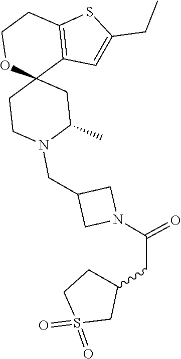 [SMILES: CCc1cc2c(s1)CCO[C@@]21CCN(CC2CN(C(=O)CC3CCS(=O)(=O)C3)C2)[C@@H](C)C1] | 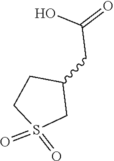 [SMILES: O=C(O)CC1CCS(=O)(=O)C1] | 423 | LCMS m/z 481.25 [M + H]⁺. |
| 466 | 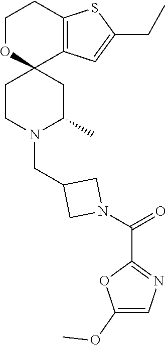 [SMILES: CCc1cc2c(s1)CCO[C@@]21CCN(CC2CN(C(=O)c3ncc(OC)o3)C2)[C@@H](C)C1] | 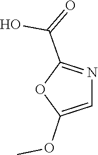 | 423 | LCMS m/z 446.17 [M + H]⁺. |
| 467 | 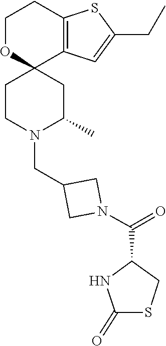 [SMILES: CCc1cc2c(s1)CCO[C@@]21CCN(CC2CN(C(=O)[C@@H]3CSC(=O)N3)C2)[C@@H](C)C1] | 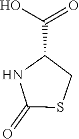 | 423 | LCMS m/z 450.17 [M + H]⁺. |
| 468 | 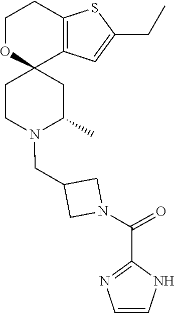 [SMILES: CCc1cc2c(s1)CCO[C@@]21CCN(CC2CN(C(=O)c3ncc[nH]3)C2)[C@@H](C)C1] | 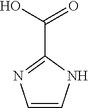 | 423 | LCMS m/z 415.33 [M + H]⁺. |
| 469 | 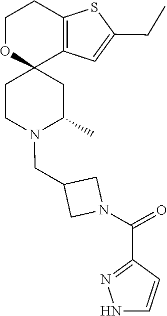 [SMILES: CCc1cc2c(s1)CCO[C@@]21CCN(CC2CN(C(=O)c3cc[nH]n3)C2)[C@@H](C)C1] | 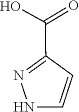 | 423 | LCMS m/z 415.06 [M + H]⁺. |
| 470 | 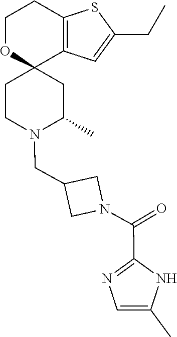 [SMILES: CCc1cc2c(s1)CCO[C@@]21CCN(CC2CN(C(=O)c3ncc(C)[nH]3)C2)[C@@H](C)C1] | 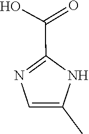 | 423 | LCMS m/z 429.28 [M + H]⁺. |
| 471 | 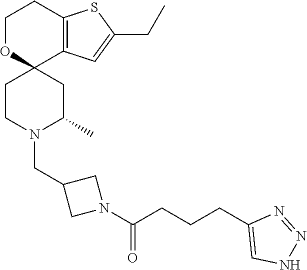 [SMILES: CCc1cc2c(s1)CCO[C@@]21CCN(CC2CN(C(=O)CCCc3c[nH]nn3)C2)[C@@H](C)C1] | 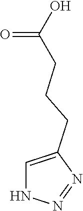 [SMILES: O=C(O)CCCc1c[nH]nn1] | 423 | LCMS m/z 458.42 [M + H]⁺. |
| 472 | 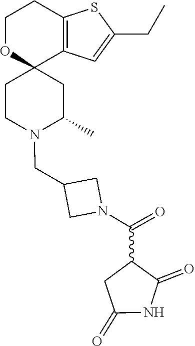 [SMILES: CCc1cc2c(s1)CCO[C@@]21CCN(CC2CN(C(=O)C3CC(=O)NC3=O)C2)[C@@H](C)C1] | 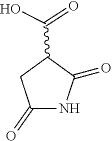 | 423 | LCMS m/z 446.30 [M + H]⁺. |
| 473 | 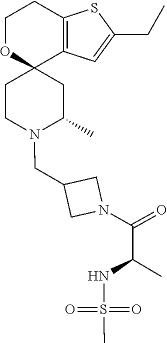 [SMILES: CCc1cc2c(s1)CCO[C@@]21CCN(CC2CN(C(=O)[C@@H](C)NS(C)(=O)=O)C2)[C@@H](C)C1] | 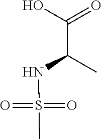 | 423 | LCMS m/z 470.24 [M + H]⁺. |
| 474 | 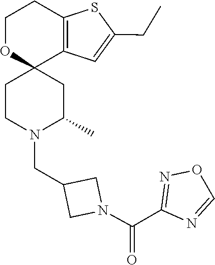 [SMILES: CCc1cc2c(s1)CCO[C@@]21CCN(CC2CN(C(=O)c3ncon3)C2)[C@@H](C)C1] | 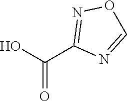 | 423 | LCMS m/z 417.05 [M + H]⁺. |
| 475 | 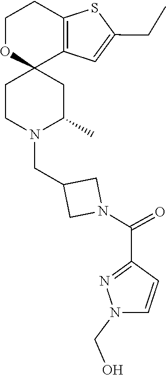 [SMILES: CCc1cc2c(s1)CCO[C@@]21CCN(CC2CN(C(=O)c3ccn(CO)n3)C2)[C@@H](C)C1] | 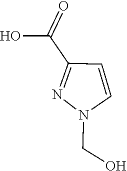 | 423 | LCMS m/z 445.39 [M + H]⁺. |
| 476 | 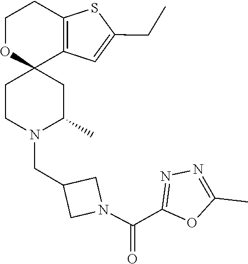 [SMILES: CCc1cc2c(s1)CCO[C@@]21CCN(CC2CN(C(=O)c3nnc(C)o3)C2)[C@@H](C)C1] | 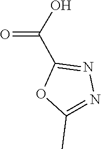 | 423 | LCMS m/z 431.18 [M + H]⁺. |
| 477 | 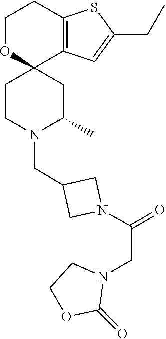 [SMILES: CCc1cc2c(s1)CCO[C@@]21CCN(CC2CN(C(=O)CN3CCOC3=O)C2)[C@@H](C)C1] | 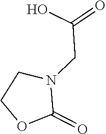 | 423 | LCMS m/z 448.22 [M + H]⁺. |
| 478 | 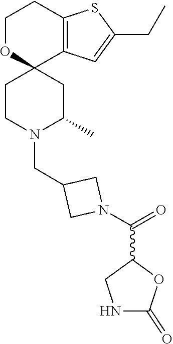 | 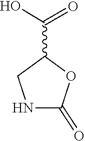 | 423 | LCMS m/z 450.17 [M + H]⁺. |
| 479 | 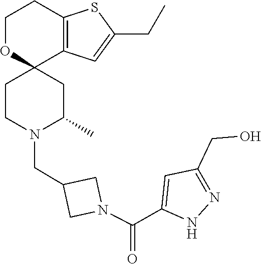 [SMILES: CCc1cc2c(s1)CCO[C@@]21CCN(CC2CN(C(=O)c3cc(CO)n[nH]3)C2)[C@@H](C)C1] | 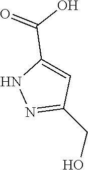 | 423 | LCMS m/z 445.19 [M + H]⁺. |
| 480 | 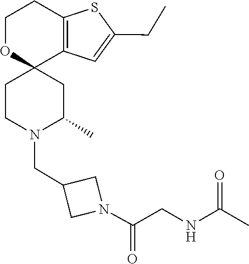 [SMILES: CCc1cc2c(s1)CCO[C@@]21CCN(CC2CN(C(=O)CNC(C)=O)C2)[C@@H](C)C1] | 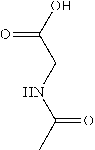 | 423 | ¹H NMR (300 MHz, Methanol- d₄) δ 6.68-6.35 (m, 1H), 4.48 (q, J = 8.3 Hz, 1H), 4.24 (q, J = 8.7, 8.3 Hz, 1H), 4.12 (dd, J = 9.0, 5.7 Hz, 1H), 4.08- 3.79 (m, 6H), 3.75-3.49 (m, 2H), 3.35 (s, 7H), 2.02 (d, J = 8.9 Hz, 9H), 1.43 (d, J = 6.5 Hz, 3H), 1.25 (t, J = 7.5 |
| | | | | Hz, 4H). LCMS |
| | | | | m/z 420.33 |
| | | | | [M + H]⁺. |
| 481 | 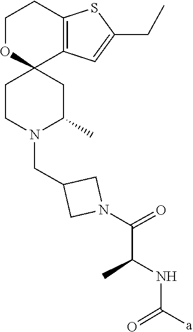 | 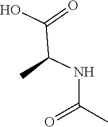 | 423 | LCMS m/z 434.25 [M + H]⁺. |
| 482 | 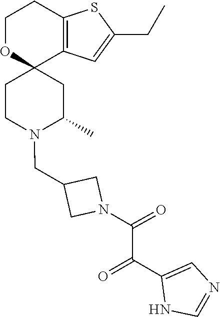 [SMILES: CCc1cc2c(s1)CCO[C@@]21CCN(CC2CN(C(=O)C(=O)c3cnc[nH]3)C2)[C@@H](C)C1] | 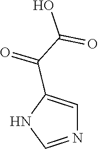 | 423 | LCMS m/z 443.28 [M + H]⁺. |
| 483 | 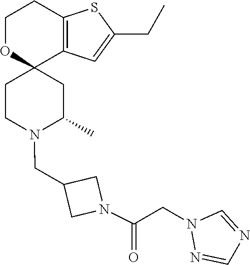 [SMILES: CCc1cc2c(s1)CCO[C@@]21CCN(CC2CN(C(=O)Cn3cncn3)C2)[C@@H](C)C1] | 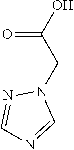 | 423 | LCMS m/z 430.04 [M + H]⁺. |
| 484 | 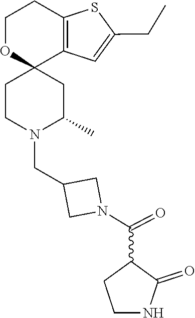 [SMILES: CCc1cc2c(s1)CCO[C@@]21CCN(CC2CN(C(=O)C3CCNC3=O)C2)[C@@H](C)C1] | 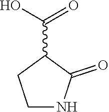 | 423 | LCMS m/z 432.34 [M + H]⁺. |
| 485 | 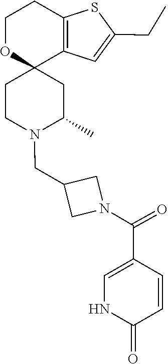 [SMILES: CCc1cc2c(s1)CCO[C@@]21CCN(CC2CN(C(=O)c3ccc(=O)[nH]c3)C2)[C@@H](C)C1] | 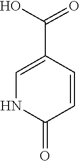 | 423 | LCMS m/z 442.16 [M + H]⁺. |
| 486 | 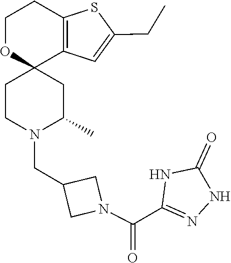 [SMILES: CCc1cc2c(s1)CCO[C@@]21CCN(CC2CN(C(=O)c3n[nH]c(=O)[nH]3)C2)[C@@H](C)C1] | 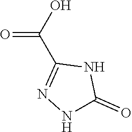 | 423 | LCMS m/z 432.19 [M + H]⁺. |
| 487 | 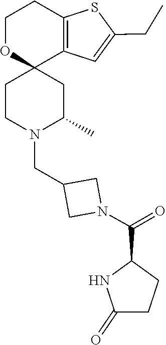 [SMILES: CCc1cc2c(s1)CCO[C@@]21CCN(CC2CN(C(=O)[C@H]3CCC(=O)N3)C2)[C@@H](C)C1] | 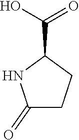 | 423 | LCMS m/z 432.25 [M + H]⁺. |
| 488 | 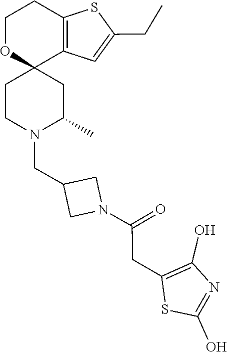 [SMILES: CCc1cc2c(s1)CCO[C@@]21CCN(CC2CN(C(=O)Cc3sc(O)nc3O)C2)[C@@H](C)C1] | 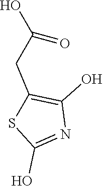 [SMILES: O=C(O)Cc1sc(O)nc1O] | 423 | LCMS m/z 483.87 [M + H]⁺. |
| 489 | 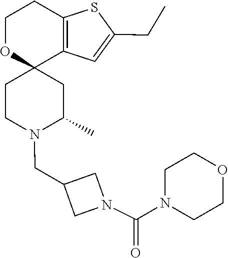 [SMILES: CCc1cc2c(s1)CCO[C@@]21CCN(CC2CN(C(=O)N3CCOCC3)C2)[C@@H](C)C1] | 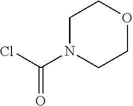 | 424 | LCMS m/z 434.25 [M + H]⁺. |
| 490 | 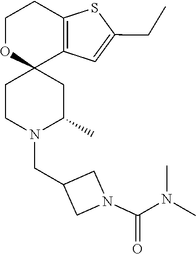 [SMILES: CCc1cc2c(s1)CCO[C@@]21CCN(CC2CN(C(=O)N(C)C)C2)[C@@H](C)C1] | 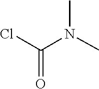 | 424 | LCMS m/z 392.26 [M + H]⁺. |

### Compound 491

### (2′S,4R)-2-ethyl-1′-[(1-ethylsulfonylazetidin-3-yl)methyl]-2′-methyl-spiro[6,7-dihydrothieno[3,2-c]pyran-4,4′-piperidine](491)

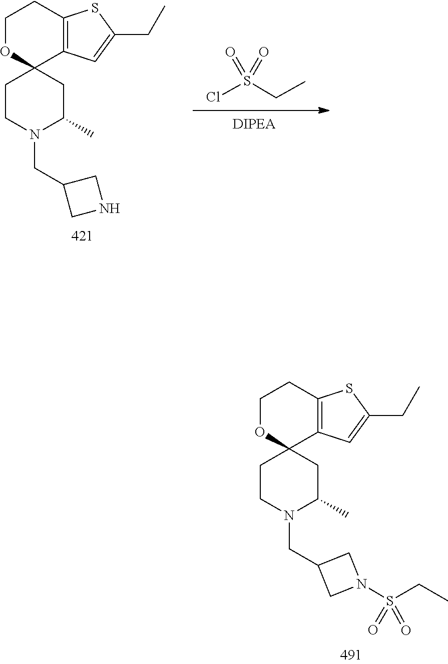

### Preparation of (2′S,4R)-2-ethyl-1′-[(1-ethylsulfonylazetidin-3-yl)methyl]-2′-methyl-spiro[6,7-dihydrothieno[3,2-c]pyran-4,4′-piperidine](491)

(2′S,4R)-1′-(azetidin-3-ylmethyl)-2-ethyl-2′-methyl-spiro[6,7-dihydrothieno[3,2-c]pyran-4,4′-piperidine](Trifluoroacetic acid (2) salt) 421 (50 mg, 0.05502 mmol) was dissolved in DMF (800 μL) and DIPEA (100 μL, 0.5740 mmol), to which ethanesulfonyl chloride (14 μL, 0.1500 mmol) was added. The reaction was stirred at room temperature for 3 hours, after which time it was diluted with MeOH (800 μL) and filtered. Purification by reversed-phase HPLC. Method: C18 Waters Sunfire column (30×150 mm, 5 micron). Gradient: MeCN in H2O with 5 mM HCl afforded the product (2′S,4R)-2-ethyl-1′-[(1-ethylsulfonylazetidin-3-yl)methyl]-2′-methyl-spiro[6,7-dihydrothieno[3,2-c]pyran-4,4′-piperidine](Hydrochloride salt) (491) (14.5 mg, 63%). LCMS m/z 413.20 [M+H]⁺.

### Compounds 492-496

Compounds 492-496 (see Table 11) were prepared in a single step from intermediate 421 using coupling method for 491. Sulfonyl chlorides were obtained from commercial sources.

TABLE 11
Structure and physicochemical data for compounds 492-496

| | | | ¹H NMR; LCMS m/z |
|---|---|---|---|
| Cmpd | Structure | Amine Reagent | [M + H]⁺ |
| 492 | 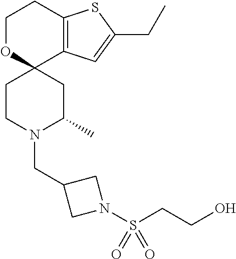 [SMILES: CCc1cc2c(s1)CCO[C@@]21CCN(CC2CN(S(=O)(=O)CCO)C2)[C@@H](C)C1] | 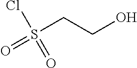 | LCMS m/z 429.07 [M + H]⁺. |
| 493 | 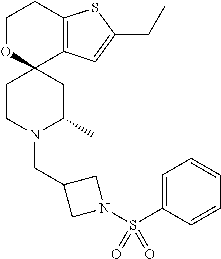 [SMILES: CCc1cc2c(s1)CCO[C@@]21CCN(CC2CN(S(=O)(=O)c3ccccc3)C2)[C@@H](C)C1] | 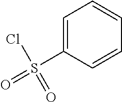 | LCMS m/z 461.15 [M + H]⁺. |
| 494 | 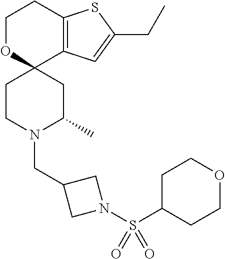 [SMILES: CCc1cc2c(s1)CCO[C@@]21CCN(CC2CN(S(=O)(=O)C3CCOCC3)C2)[C@@H](C)C1] | 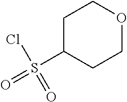 | LCMS m/z 469.26 [M + H]⁺. |
| 495 | 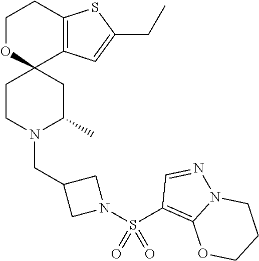 [SMILES: CCc1cc2c(s1)CCO[C@@]21CCN(CC2CN(S(=O)(=O)c3cnn4c3OCCC4)C2)[C@@H](C)C1] | 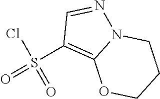 [SMILES: O=S(=O)(Cl)c1cnn2c1OCCC2] | LCMS m/z 507.18 [M + H]⁺. |
| 496 | 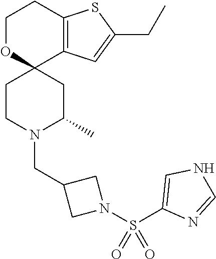 [SMILES: CCc1cc2c(s1)CCO[C@@]21CCN(CC2CN(S(=O)(=O)c3c[nH]cn3)C2)[C@@H](C)C1] | 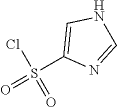 | LCMS m/z 451.25 [M + H]⁺. |

### Compound 497

### (2′S,4R)-2-ethyl-2′-methyl-1′-[[1-(2-methylsulfonylethyl)triazol-4-yl]methyl]spiro[6,7-dihydrothieno[3,2-c]pyran-4,4′-piperidine]1,1-dioxide

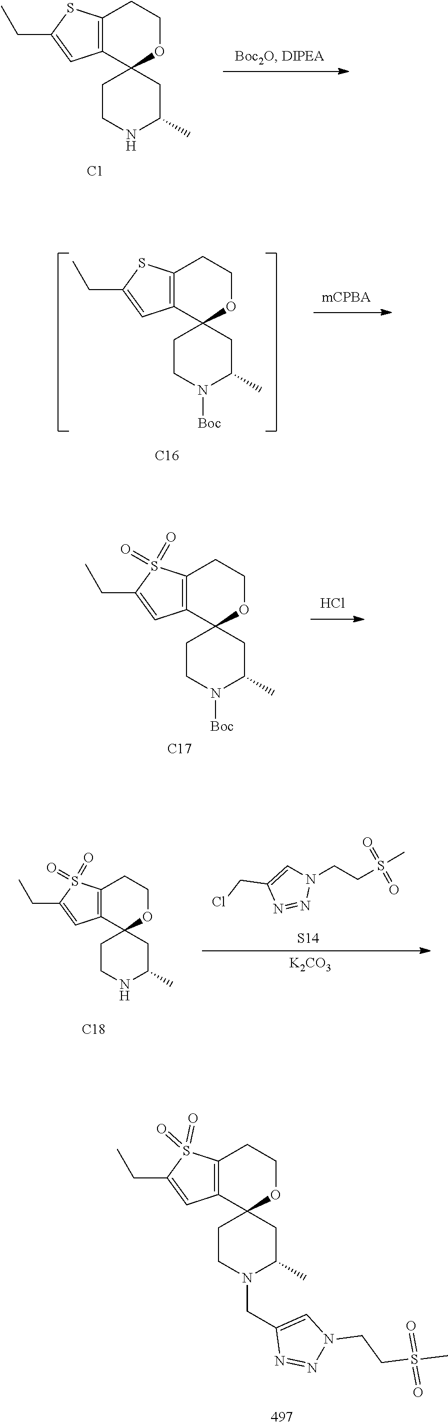

### Step 1. Synthesis of tert-butyl (2′S,4R)-2-ethyl-2′-methyl-spiro[6,7-dihydrothieno[3,2-c]pyran-4,4′-piperidine]-1′-carboxylate (C16)

To a mixture of (2′S,4R)-2-ethyl-2′-methyl-spiro[6,7-dihydrothieno[3,2-c]pyran-4,4′-piperidine](200 mg, 0.7956 mmol) C1 in DCM (6 mL) was added Boc₂O (211 μL, 0.9185 mmol) and DIPEA (160 μL, 0.9186 mmol). After 1 h, the reaction mixture was diluted with sat. aq. ammonium chloride. The layers were separated, and the organic layer was passed over a phase separator and used in the next step without further purification.

### Step 2. Synthesis of tert-butyl (2′S,4R)-2-ethyl-2′-methyl-1,1-dioxo-spiro[6,7-dihydrothieno[3,2-c]pyran-4,4′-piperidine]-1′-carboxylate (C17)

To a mixture of tert-butyl (2′S,4R)-2-ethyl-2′-methyl-spiro[6,7-dihydrothieno[3,2-c]pyran-4,4′-piperidine]-1′-carboxylate C16 from the previous step in DCM (6 mL) was added mCPBA (200 mg, 0.8692 mmol) and the reaction was stirred at rt. The mixture was then divided in two halves and one half of the mixture was stirred with mCPBA (300 mg, 1.74 mmol) at reflux for 3 h. At this time, the mixture was cooled to rt, diluted with sat. sodium bicarbonate (20 mL) and additional DCM (20 mL). The layers were separated, and the aqueous layer was washed with DCM (10 mL), and then the combined organic layers were washed with additional sat. sodium bicarbonate (10 mL) followed by brine (10 mL). The organic layer was dried with sodium sulfate, filtered, and concentrated. Purification by silica gel chromatography (Gradient: 0-100% EtOAc in heptane) afforded tert-butyl (2′S,4R)-2-ethyl-2′-methyl-1,1-dioxo-spiro[6,7-dihydrothieno[3,2-c]pyran-4,4′-piperidine]-1′-carboxylate C17 (46 mg, 26%). ¹H NMR (400 MHz, Chloroform-d₃) δ 6.14 (t, J=2.1 Hz, 1H), 4.01 (tt, J=11.8, 6.5 Hz, 1H), 3.95-3.67 (m, 3H), 3.24 (ddd, J=14.0, 9.3, 5.4 Hz, 1H), 2.63-2.40 (m, 4H), 1.95-1.74 (m, 2H), 1.70-1.56 (m, 2H), 1.46 (d, J=14.0 Hz, 9H), 1.31-1.23 (m, 6H). LCMS m/z 383.85 [M+H]⁺.

### Step 3. Synthesis of (2′S,4R)-2-ethyl-2′-methyl-spiro[6,7-dihydrothieno[3,2-c]pyran-4,4′-piperidine]1,1-dioxide (C18)

To tert-butyl (2′S,4R)-2-ethyl-2′-methyl-1,1-dioxo-spiro[6,7-dihydrothieno[3,2-c]pyran-4,4′-piperidine]-1′-carboxylate C17 (46 mg, 0.12 mmol) was added HCl (500 μL of 4 M, 2.000 mmol) in dioxane. The mixture was stirred at rt. After 1 h, the mixture was concentrated in vacuo to provide (2′S,4R)-2-ethyl-2′-methyl-spiro[6,7-dihydrothieno[3,2-c]pyran-4,4′-piperidine]1,1-dioxide C18 (Hydrochloride salt) (33 mg, 25%). LCMS m/z 284.03 [M+H]⁺. It was used in the next step without further purification.

### Step 4. Synthesis of (2′S,4R)-2-ethyl-2′-methyl-1′-[[1-(2-methylsulfonylethyl)triazol-4-yl]methyl]spiro[6,7-dihydrothieno[3,2-c]pyran-4,4′-piperidine]1,1-dioxide (497)

To a mixture of (2′S,4R)-2-ethyl-2′-methyl-spiro[6,7-dihydrothieno[3,2-c]pyran-4,4′-piperidine]1,1-dioxide C (Hydrochloride salt) (33 mg, 0.1032 mmol) in acetonitrile (3 mL) was added potassium carbonate (30 mg, 0.2171 mmol) and 4-(chloromethyl)-1-(2-methylsulfonylethyl)triazole (30 mg, 0.1155 mmol). After stirring for 60 h, the reaction mixture was blown dry, diluted with water/DMSO and the mixture was purified by reverse phase HPLC (C18 column, gradient: 10-100% MeCN in Water, with TFA as the modifier). The product-containing fractions were pooled, concentrated, and diluted with 6 N NaOH/DCM. The layers were separated, and the aqueous layer was extracted with additional DCM. The organic layer was passed over a phase separator and concentrated to yield (2′S,4R)-2-ethyl-2′-methyl-1′-[[1-(2-methylsulfonylethyl)triazol-4-yl]methyl]spiro[6,7-dihydrothieno[3,2-c]pyran-4,4′-piperidine]1,1-dioxide 497 (12 mg, 23%). ¹H NMR (400 MHz, Chloroform-d₃) δ 7.64 (s, 1H), 6.24 (t, J=2.2 Hz, 1H), 4.92-4.83 (m, 2H), 4.04 (d, J=14.7 Hz, 1H), 3.88 (d, J=14.7 Hz, 1H), 3.84-3.78 (m, 1H), 3.78-3.70 (m, 3H), 2.73 (s, 3H), 2.63-2.46 (m, 6H), 1.83-1.62 (m, 4H), 1.61-1.48 (m, 1H), 1.30-1.23 (m, 6H). LCMS m/z 471.09 [M+H]⁺.

### Compound 498

### 2-ethyl-2′,2′-dimethyl-1′-[(1-methylpyrazol-4-yl)methyl]spiro[6,7-dihydrothieno[3,2-c]pyran-4,4′-piperidine]

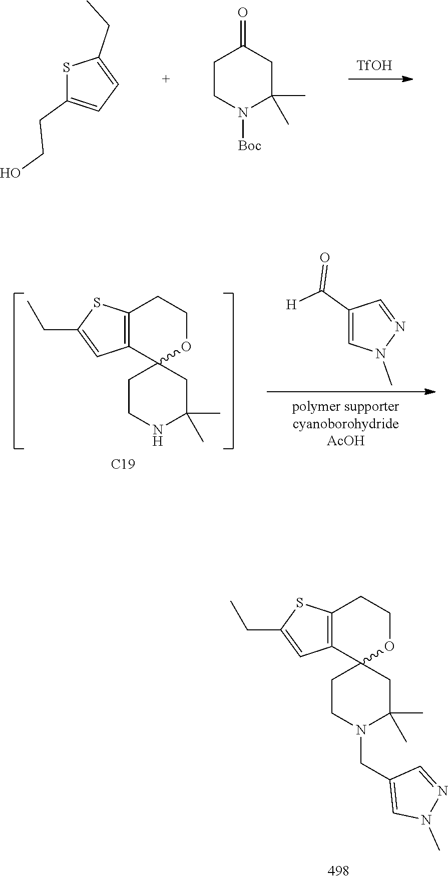

### Step 1. Synthesis of 2-ethyl-2′,2′-dimethyl-spiro[6,7-dihydrothieno[3,2-c]pyran-4,4′-piperidine](C19)

To a vial was added tert-butyl 2,2-dimethyl-4-oxo-piperidine-1-carboxylate (90.9 mg, 0.4 mmol), 2-(5-ethyl-2-thienyl)ethanol (75 mg, 0.48 mmol) and dioxane (2 mL). Then trifluoromethanesulfonic acid (100 μL, 1.13 mmol) was added at 0° C., and stirred at rt for 4 h. The reaction mixture was concentrated in vacuo to provide 2-ethyl-2′,2′-dimethyl-spiro[6,7-dihydrothieno[3,2-c]pyran-4,4′-piperidine] C19, which was used in the next step without purification.

### Step 2. Synthesis of 2-ethyl-2′,2′-dimethyl-1′-[(1-methylpyrazol-4-yl)methyl]spiro[6,7-dihydrothieno[3,2-c]pyran-4,4′-piperidine](498)

To the crude material from the previous step in DCM were added AcOH (114 μL, 2.0 mmol), 1-methylpyrazole-4-carbaldehyde (88 mg, 0.8 mmol), and cyanoborohydride, polymer supported (500 mg, 1 mmol). The reaction mixture was heated in a microwave reactor at 110° C. for 20 min. The reaction mixture was then filtered, concentrated in vacuo. Purification by reversed-phase HPLC. Method: C18 Waters Sunfire column (30×150 mm, 5 micron). Gradient: MeCN in H₂O with 0.1% trifluoroacetic acid. Product was isolated as 2-ethyl-2′,2′-dimethyl-1′-[(1-methylpyrazol-4-yl)methyl]spiro[6,7-dihydrothieno[3,2-c]pyran-4,4′-piperidine](trifluoroacetic acid salt) (20.5 mg, 11%). LCMS m/z 360.23 [M+H]⁺.

### Preparation S15

### 2-(4,5-dimethyl-2-thienyl)ethanol (S15)

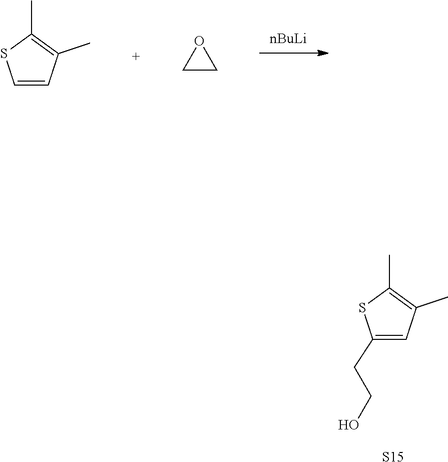

### Preparation of 2-(4,5-dimethyl-2-thienyl)ethanol (S15)

To a solution of 2,3-dimethylthiophene (2 g, 17.826 mmol) in Et₂O (50 mL) at 0° C. was added n-BuLi (8.5564 mL of 2.5 M, 21.391 mmol) over 15 min. The mixture was stirred at rt for 30 minutes. After cooling to 0° C., a solution of oxirane (7.1303 mL of 3 M, 21.391 mmol) was added. The reaction was stirred at 0° C. for 3 h then quenched with water and extracted with EtOAc. The organic layer was dried over anhydrous sodium sulfate, filtered, and concentrated in vacuo. Purification by silica gel chromatography (Gradient: 20-25% EtOAc in hexanes) afforded 2-(4,5-dimethyl-2-thienyl)ethanol S15 (2 g, 67%). ¹H NMR (400 MHz, DMSO-d₆) δ 6.51 (s, 1H), 4.71 (s, 1H), 3.54 (s, 2H), 2.77 (t, J=6.4 Hz, 2H), 2.21 (s, 3H), 2.00 (s, 3H). LCMS m/z 157.1 [M+H]⁺.

### Intermediates S16-S17

Intermediates S16-S17 (see Table 12) were prepared in a single step the corresponding thiophenes using the method described for S15. Any modifications to methods are noted in Table 11 and accompanying footnotes.

TABLE 12
Structure and physicochemical data for compounds S16-S17

| Inter- | | Thiophene | ¹H NMR; LCMS m/z |
|---|---|---|---|
| mediate | Product | Reagent | [M + H]⁺ |
| S16 | 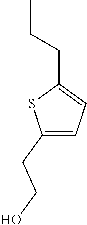 | 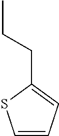 | ¹H NMR (400 MHz, DMSO-d₆) δ 6.65 (d, J = 3.24 Hz, 1H), 6.59 (d, J = 3.24 Hz, 1H), 3.84-3.79 (m, 2H), 2.9 (t, J = 8 Hz, 2H), 2.72 (t, J = 8 Hz, 1H), 1.71-1.63 (m, 3H), 0.49 (t, J = 7.08 Hz, 3H). |
| S17 | 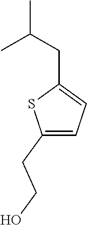 | 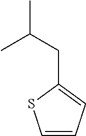 | ¹H NMR (400MHz, DMSO-d₆) δ 6.64 (d, J = 2.92 Hz, 1H), 6.58 (d, J = 2.96 Hz, 1H), 4.74 (s, 1H), 3.57 (t, J = 6.44 Hz, 2H), 2.84 (t, J = 6.8 Hz, 2H), 2.56 (d, J = 6.96 Hz, 2H), 1.82- 1.72 (m, 1H), 0.88 (d, J = 6.56 Hz, 6H). LCMS m/z 185.1 [M + H]⁺. |

### Preparation S18

### (2′S,4R)-2,2′-dimethylspiro[6,7-dihydrothieno[3,2-c]pyran-4,4′-piperidine](S18)

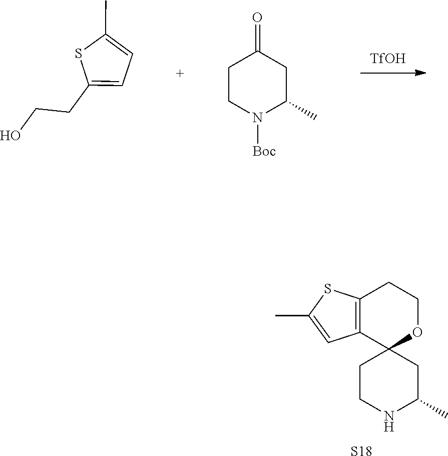

### Preparation of (2′S,4R)-2,2′-dimethylspiro[6,7-dihydrothieno[3,2-c]pyran-4,4′-piperidine](S18)

To a flask was prepared a solution of 2-(5-methyl-2-thienyl)ethanol (12.8 g, 85.50 mmol) and tert-butyl (2S)-2-methyl-4-oxo-piperidine-1-carboxylate (20 g, 93.78 mmol, 1.1 eq) in dioxane (158 mL). The flask was cooled with an ice-methanol bath allowed to equilibrate for 10 min, reaching an internal temperature of ˜0° C. Trifluoromethanesulfonic acid (17 mL, 192.1 mmol, 2.2 eq) was added dropwise via addition funnel over 30 min. After the addition, the mixture was stirred for another 30 min with the ice bath. Then the ice bath was removed, and the reaction was allowed to warm to rt and stirred overnight. The mixture was diluted with a 1:1 mixture of water and saturated sodium bicarbonate aqueous solution (500 mL) and EtOAc (400 mL). The layers were separated the aqueous layer was re-extracted with EtOAc (200 mL). The combined organic layers were dried over sodium sulfate, filtered, and concentrated in vacuo. The solids were then suspended in Et₂O (˜250 mL) at rt and then heated to reflux. The mixture was dissolved while refluxing and then cooled. Heptane was added, followed by more Et₂O. Part of the solvent was removed. Solids were filtered and washed with 1:1 Et₂O-heptane (3×75 mL) and further dried under high vacuum. Solids were suspended in Et₂O (250 mL) and was stirred and heated to reflux for 30 min. At this time, the mixture was cooled to 0° C. and stirred for 10 min, filtered, and then rinsed with ice-cold Et₂O to yield a colorless solid, which was dried under air for 5 m and then at high vacuum overnight. (2′S,4R)-2,2′-dimethylspiro[6,7-dihydrothieno[3,2-c]pyran-4,4′-piperidine](trifluoromethanesulfonate) S18 (19.3 g, 570%) ¹H NMR (300 MHz, Chloroform-d) δ 7.78 (bs, 1H), 7.48 (bs, 1H), 6.52 (d, J=1.3 Hz, 1H), 3.89 (hept, J=5.8 Hz, 2H), 3.75-3.54 (m, 1H), 3.40 (dq, 8=10.9, 4.7, 3.8 Hz, 2H), 2.77 (td, J=5.2, 2.8 Hz, 2H), 2.43 (d, J=1.1 Hz, 3H), 2.21 (ddd, J=15.0, 11.5, 6.4 Hz, 1H), 2.09-1.92 (m, 3H), 1.42 (d, J=6.6 Hz, 3H). LCMS m/z 238.14 [M+H]⁺.

### Key Intermediates S19-S21

Intermediates S19-S21 (see Table 13) were prepared in a single step from tert-butyl (S)-2-methyl-4-oxopiperidine-1-carboxylate and the appropriate thiophene ethanol reagent S15-S17 using Pictet-Spengler reaction as in the preparation of intermediate S18. Any modifications to methods are noted in Table 13 and accompanying footnotes.

TABLE 13
Structure and physicochemical data for compounds S19-S21

| | | Thiophene | |
|---|---|---|---|
| Intermediate | Product | Ethanol Reagent | ¹H NMR; LCMS m/z [M + H]⁺ |
| S19¹ | 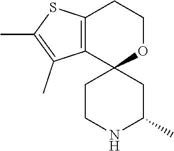 | 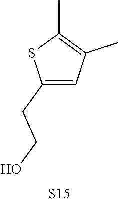 | ¹H NMR (400 MHz, DMSO-d₆) δ 8.36 (bs, 2H), 3.88-3.78 (m, 2H), 3.43-3.39 (m, 1H), 3.23-3.09 (m, 2H), 2.68 (t, J = 5.12 Hz, 2H), 2.23-2.19 (m, 3H), 2.17-2.12 (m, 4H), 1.98 (t, J = 14.36 Hz, 1H), 1.84 (t, J = 18.84 Hz, 2H), 1.20 (d, J = 6.48 Hz, 3H); LCMS m/z 251.9 [M + H]⁺. |
| S20² | 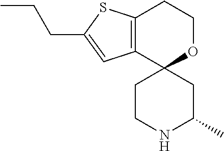 [SMILES: CCCc1cc2c(s1)CCO[C@@]21CCN[C@@H](C)C1] | 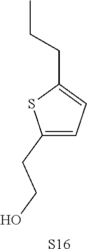 | ¹H NMR (400 MHz, DMSO-d₆) δ 8.57 (bs, 1H), 8.19 (d, J = 8 Hz, 1H), 6.45 (s, 1H), 3.84 (d, J = 4.12 Hz, 2H), 3.35 (bs, 1H), 3.22 (d, J = 12 Hz, 1H), 3.09 (bs, 1H), 2.69 (t, J = 6.4 Hz, 4H), 1.98-1.91 (m, 3H), 1.81-1.74 (m, 1H), 1.61- 1.55 (m, 2H), 1.19 (d, J = 4 Hz, 3H), 0.922 (t, J = 7.2 Hz, 3H); LCMS m/z 266.3 [M + H]⁺ |
| S21³ | 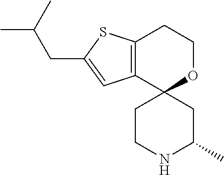 [SMILES: CC(C)Cc1cc2c(s1)CCO[C@@]21CCN[C@@H](C)C1] | 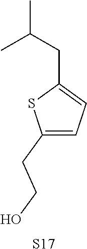 | ¹H NMR (400 MHz, DMSO-d₆) δ 8.50 (bs, 1H), 6.42 (s, 1H), 3.87 (d, J = 4.28 Hz, 2H), 3.32 (bs, 1H), 3.23-3.20 (m, 1H), 3.10 (bs, 1H), 2.70 (bs, 2H), 2.59 (d, J = 6.88 Hz, 2H), 1.98-1.87 (m, 3H), 1.80- 1.73 (m, 2H), 1.20 (d, J = 6.4 Hz, 3H), 0.90 (d, J = 6.52 Hz, 6H); LCMS m/z 279.8 [M + H]⁺. |

Footnotes:
¹Tert-butyl (2S)-2-methyl-4-oxo-piperidine-1-carboxylate (0.9 eq) and trifluoromethanesulfonic acid (2.8 eq) were used.
²Tert-butyl (2S)-2-methyl-4-oxo-piperidine-1-carboxylate (0.8 eq) and trifluoromethanesulfonic acid (3.0 eq) were used.
³Tert-butyl (2S)-2-methyl-4-oxo-piperidine-1-carboxylate (0.9 eq) and trifluoromethanesulfonic acid (2.8 eq) were used.

### Preparation S21

### tert-butyl (2′S)-2′-methylspiro[6,7-dihydrothieno[3,2-c]pyran-4,4′-piperidine]-1′-carboxylate (S21)

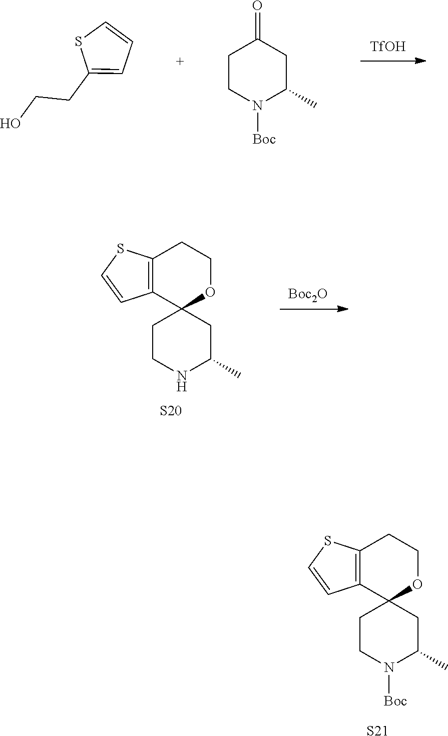

### Step 1: (2′S)-2′-methylspiro[6,7-dihydrothieno[3,2-c]pyran-4,4′-piperidine](S20)

A solution of 2-(2-thienyl)ethanol (1.028 g, 7.618 mmol) and tert-butyl (2S)-2-methyl-4-oxo-piperidine-1-carboxylate (1.8 g, 8.440 mmol) in dioxane (15 mL) was cooled an ice bath. To the solution was added trifluoromethanesulfonic acid (2.5 mL, 28.25 mmol) over 5 minutes. The resulting solution was slowly warmed to rt and stirred overnight, after which time the reaction was basified to pH 8 with 1N NaOH. The mixture was partitioned with EtOAc, and the combined organics were dried over anhydrous sodium sulfate, filtered and concentrated in vacuo to provide an amber colored crude oil. Purification by HPLC: 10-90% ACN in Water (TFA modifier) C18 column followed by lyophilization to afford (2′S)-2′-methylspiro[6,7-dihydrothieno[3,2-c]pyran-4,4′-piperidine](trifluoromethanesulfonate) (1.40 g). ¹H NMR (300 MHz, Chloroform-d) δ 7.13 (d, J=5.2 Hz, 1H), 6.87 (d, J=5.3 Hz, 1H), 3.97-3.86 (m, 2H), 3.62 (d, J=15.1 Hz, 1H), 3.39 (d, J=8.4 Hz, 2H), 2.84 (td, J=5.3, 2.0 Hz, 2H), 2.31-2.18 (m, 1H), 2.07-1.97 (m, 3H), 1.41 (d, J=6.6 Hz, 3H). LCMS m/z 224.18 [M+H]⁺.

### Step 2: tert-butyl (2′S)-2′-methylspiro[6,7-dihydrothieno[3,2-c]pyran-4,4′-piperidine]-1′-carboxylate (S21)

The material obtained was dissolved in DCM (20 mL) and treated with Boc₂O (1.9 mL, 8.270 mmol) followed by DIPEA (2.7 mL, 15.50 mmol). The resulting solution was stirred at rt overnight. The reaction was partitioned between 1N NaOH and DCM. The organics were collected through a phase separator tube, concentrated in vacuo, followed by purification by silica gel chromatography (Gradient: 0-50% EtOAc in heptane) to afford tert-butyl (2′S)-2′-methylspiro[6,7-dihydrothieno[3,2-c]pyran-4,4′-piperidine]-1′-carboxylate S21 (786.4 mg, 32%). ¹H NMR (300 MHz, Chloroform-d) δ 7.10 (dd, J=5.2, 0.7 Hz, 1H), 6.73 (d, J=5.2 Hz, 1H), 4.06-3.95 (m, 1H), 3.93-3.88 (m, 2H), 3.81-3.72 (m, 1H), 3.37 (ddd, J=14.0, 8.8, 5.4 Hz, 1H), 2.82 (q, J=5.2 Hz, 2H), 2.11-1.93 (m, 2H), 1.86-1.73 (m, 2H), 1.50 (s, 9H), 1.28 (d, J=6.6 Hz, 3H).

### Preparation S22

### (2′R,4R)-2,2′-dimethylspiro[6,7-dihydrothieno[3,2-c]pyran-4,4′-piperidine](S22)

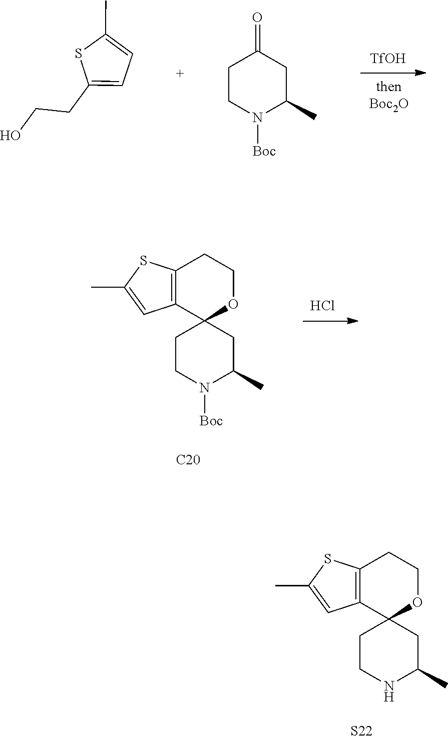

### Step 1. Synthesis of tert-butyl (2′R,4R)-2,2′-dimethylspiro[6,7-dihydrothieno[3,2-c]pyran-4,4′-piperidine]-1′-carboxylate (C20)

A solution of 2-(5-methyl-2-thienyl)ethanol (107 mg, 0.7524 mmol) and tert-butyl (2R)-2-methyl-4-oxo-piperidine-1-carboxylate (170 mg, 0.7971 mmol) in DCM (1.4 mL) was cooled to −78° C. To the reaction mixture, trifluoromethanesulfonic acid (130 μL, 1.469 mmol) was added and the reaction was stirred at −78° C. for 1.5 h. Reaction was quenched into a biphasic mixture of sat. NaHCO₃ (15 mL) and DCM (15 mL) and the aqueous layer pH was adjusted to >10 with 2N NaOH. The aqueous layer was then extracted with DCM (3×15 mL) and filtered through an phase separation cartridge and concentrated. The crude material was dissolved in DCM (1.4 mL) and triethyl amine (250 μL, 1.794 mmol) and di-tert-butyl dicarbonate (250 μL, 1.088 mmol) were added to the reaction in that order. Reaction was stirred at rt for 18 h. The solution was diluted with DCM and washed with water (10 mL). The aqueous layer was extracted with DCM (2×10 mL), dried over sodium sulfate, filtered through a phase separation filter, and concentrated in vacuo. The crude mixture was concentrated and purified by silica gel chromatography (Gradient: 0-30% EtOAc in heptane) to yield tert-butyl (2′R,4R)-2,2′-dimethylspiro[6,7-dihydrothieno[3,2-c]pyran-4,4′-piperidine]-1′-carboxylate C20 (138.3 mg, 54%). ¹H NMR (300 MHz, Methanol-d₄) δ 6.42 (d, J=1.2 Hz, 1H), 4.35 (d, J=7.2 Hz, 1H), 3.99-3.82 (m, 3H), 2.83-2.60 (m, 2H), 2.37 (s, 3H), 1.89 (d, J=4.3 Hz, 2H), 1.85-1.75 (m, 2H), 1.48 (s, 9H), 1.33 (d, J=7.1 Hz, 3H).

### Step 2. Synthesis of (2′R,4R)-2,2′-dimethylspiro[6,7-dihydrothieno[3,2-c]pyran-4,4′-piperidine](S22)

To a stirred solution of tert-butyl (2′R,4R)-2,2′-dimethylspiro[6,7-dihydrothieno[3,2-c]pyran-4,4′-piperidine]-1′-carboxylate C20 (135 mg, 0.4000 mmol) in DCM (1.3 mL) was added hydrogen chloride in dioxanes (500 μL of 4 M, 2.000 mmol) and the reaction was stirred for 2.5 h at rt. The reaction was concentrated under a stream of nitrogen. Purification by reversed-phase HPLC. Method: C18 Waters Sunfire column (30×150 mm, 5 micron). Gradient: MeCN in H₂O with 0.1% HCl. (2′R,4R)-2,2′-dimethylspiro[6,7-dihydrothieno[3,2-c]pyran-4,4′-piperidine](Hydrochloride salt) S22 (84.9 mg, 71%). ¹H NMR (300 MHz, Methanol-d₄) δ 6.52 (s, 1H), 3.94 (t, J=5.4 Hz, 2H), 3.83-3.67 (m, 1H), 3.50 (td, J=12.5, 3.9 Hz, 1H), 3.19 (dt, J=13.0, 4.1 Hz, 1H), 2.83-2.63 (m, 2H), 2.41 (d, J=1.1 Hz, 3H), 2.24-1.96 (m, 4H), 1.55 (d, J=7.1 Hz, 3H). LCMS m/z 238.0 [M+H]⁺.

### Preparation S23

### (2′S,4S)-2,2′-dimethylspiro[6,7-dihydrothieno[3,2-c]pyran-4,4′-piperidine](S23)

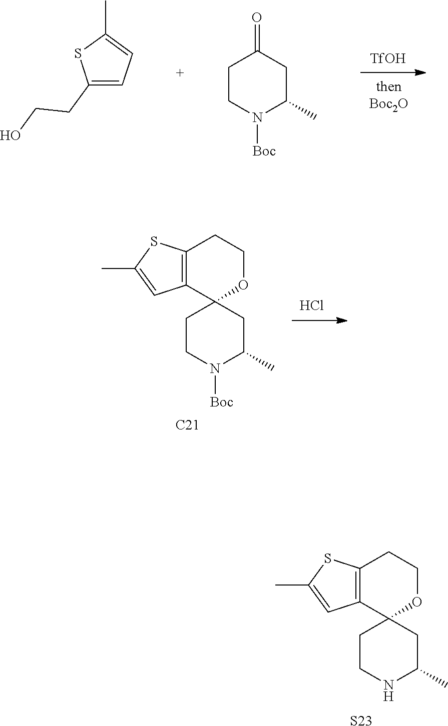

(2′S,4S)-2,2′-dimethylspiro[6,7-dihydrothieno[3,2-c]pyran-4,4′-piperidine]S23 was prepared using the same procedures as in the preparation of S22. ¹H NMR (300 MHz, Methanol-d₄) δ 6.51 (s, 1H), 3.94 (t, J=5.4 Hz, 2H), 3.81-3.69 (m, 1H), 3.50 (td, J=12.2, 3.6 Hz, 1H), 3.19 (dt, J=13.0, 4.1 Hz, 1H), 2.74 (td, J=5.5, 1.7 Hz, 2H), 2.41 (s, 3H), 2.22-1.96 (m, 4H), 1.55 (d, J=7.1 Hz, 3H). LCMS m/z 238.0 [M+H]⁺.

### Preparation S24

### (2′S)-2-chloro-2′-methyl-spiro[6,7-dihydrothieno[3,2-c]pyran-4,4′-piperidine](S24)

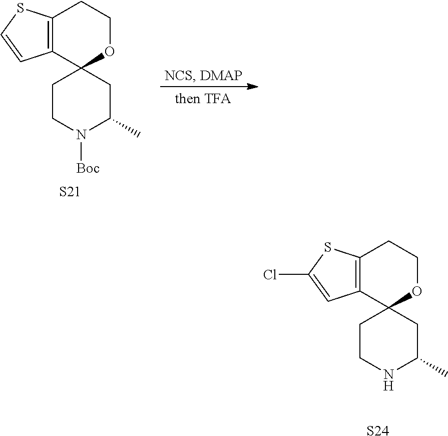

A solution of tert-butyl (2′S)-2′-methylspiro[6,7-dihydrothieno[3,2-c]pyran-4,4′-piperidine]-1′-carboxylate S21 (286 mg, 0.8842 mmol) in ACN (4.5 mL) was treated with NCS (112 mg, 0.8387 mmol) and DMAP (1.1 mg, 0.009004 mmol). The resulting solution was stirred overnight then purified by silica gel chromatography (Gradient: 10-100% EtOAc in heptane) to afford tert-butyl (2′S)-2-chloro-2′-methyl-spiro[6,7-dihydrothieno[3,2-c]pyran-4,4′-piperidine]-1′-carboxylate (210 mg, 66%). LCMS m/z 358.22 [M+H]+.

The product was dissolved in DCM (4 mL) and treated with TFA (210 μL, 2.726 mmol). The resulting solution was stirred at rt for 3 h, followed by concentration in vacuo and coevaporated with MeOH to afford (2′S)-2-chloro-2′-methyl-spiro[6,7-dihydrothieno[3,2-c]pyran-4,4′-piperidine]S24 (Trifluoroacetate salt) (180 mg, 46%). LCMS m/z 258.21 [M+H]⁺.

### Compounds 499 [ENANT-1] and 500 [ENANT-2]

### 4-[(2′S,4R)-2,2′-dimethylspiro[6,7-dihydrothieno[3,2-c]pyran-4,4′-piperidine]-1′-yl]tetrahydrofuran-3-ol

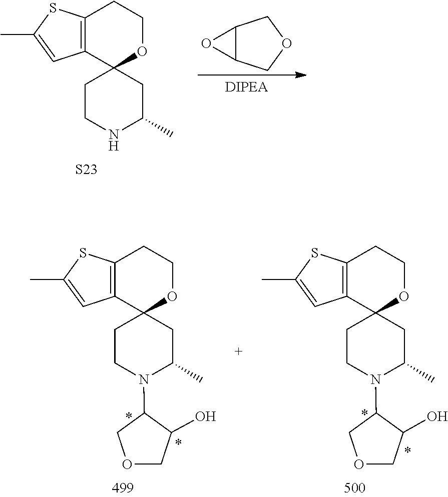

### Preparation of 4-[(2′S,4R)-2,2′-dimethylspiro[6,7-dihydrothieno[3,2-c]pyran-4,4′-piperidine]-1′-yl]tetrahydrofuran-3-ol (499 [ENANT-1] and 500 [ENANT-2])

(2′S,4R)-2,2′-dimethylspiro[6,7-dihydrothieno[3,2-c]pyran-4,4′-piperidine](trifluoromethanesulfonate) S23 (54 mg, 0.1397 mmol), DIPEA (210 μL, 1.206 mmol, 8.6 eq) and 3,6-dioxabicyclo[3.1.0]hexane (43 μL, 0.60 mmol, 4.3 eq) were mixed with n-BuOH (1 ml). And the resulting mixture was heated at 210° C. for 1 h. The crude reaction mixture was evaporated. Purification by reversed-phase HPLC. Method: C18 Waters Sunfire column (30×150 mm, 5 micron). Gradient: MeCN in H₂O with 0.1% formic acid. Two products were isolated:

4-[(2′S,4R)-2,2′-dimethylspiro[6,7-dihydrothieno[3,2-c]pyran-4,4′-piperidine]-1′-yl]tetrahydrofuran-3-ol (499[ENANT-1]) (formic acid) (16 mg, 62%). ¹H NMR (300 MHz, Methanol-d₄) δ 8.44 (s, 1H), 6.50 (d, J=1.3 Hz, 1H), 4.69 (t, J=6.1 Hz, 1H), 4.20 (dd, J=9.6, 6.6 Hz, 1H), 4.15-4.02 (m, 3H), 4.02-3.87 (m, 2H), 3.61 (dt, J=10.8, 4.1 Hz, 1H), 3.49 (dd, J=9.6, 5.5 Hz, 1H), 3.30-3.21 (m, 1H), 3.09 (td, J=12.0, 4.2 Hz, 1H), 2.73 (td, J=5.3, 2.1 Hz, 2H), 2.40 (d, J=1.0 Hz, 3H), 2.19-1.89 (m, 4H), 1.40 (d, J=6.4 Hz, 3H). LCMS m/z 324.26 [M+H]⁺; and

4-[(2′S,4R)-2,2′-dimethylspiro[6,7-dihydrothieno[3,2-c]pyran-4,4′-piperidine]-1′-yl]tetra-hydrofuran-3-ol (500 [ENANT-2]) (formic acid) (12 mg, 46%). ¹H NMR (300 MHz, Methanol-d₄) δ 8.38 (s, 1H), 6.53 (q, J=1.1 Hz, 1H), 4.70 (t, J=5.3 Hz, 1H), 4.30-4.07 (m, 2H), 4.07-3.88 (m, 4H), 3.70-3.48 (m, 2H), 3.28-3.20 (m, 1H), 3.15 (dd, J=12.2, 2.7 Hz, 1H), 2.73 (t, J=5.4 Hz, 2H), 2.40 (d, J=1.0 Hz, 3H), 2.24-1.90 (m, 4H), 1.36 (d, J=6.5 Hz, 3H). LCMS m/z 324.26 [M+H]⁺.

### Compounds 501-507

Compounds 501-507 (see Table 14) were prepared in a single step from intermediate S23 or S24 as in the preparation of compound 499 and 500. Epoxides were obtained from commercial sources. Any modifications to methods are noted in Table 14 and accompanying footnotes.

TABLE 14
Structure and physicochemical data for compounds 501-507

| | | Epoxide | Starting | |
|---|---|---|---|---|
| Cmpd | Structure | Reagent | Material | ¹H NMR; LCMS m/z [M + H]⁺ |
| 501 | 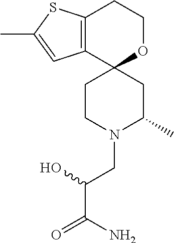 | 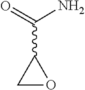 | S23¹ | ¹H NMR (300 MHz, Chloroform-d) δ 7.28 (s, 1H), 6.44 (t, J = 1.3 Hz, 1H), 5.71 (s, 1H), 4.22-4.15 (m, 1H), 3.91 (qdd, J = 8.8, 4.9, 2.0 Hz, 2H), 3.36-2.58 (m, 6H), 2.43 (s, 3H), 1.97-1.77 (m, 3H), 1.66 (dd, J = 14.1, 11.2 Hz, 1H), 1.11 (dd, J = 11.3, 6.3 Hz, 3H); LCMS m/z 326.18 [M + H]⁺. |
| 502 | 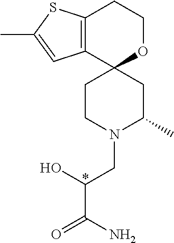 | 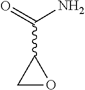 | S23² | ¹H NMR (300 MHz, Methanol-d₄) δ 6.47 (q, J = 1.1 Hz, 1H), 4.21 (dd, J = 7.6, 3.9 Hz, 1H), 3.89 (td, J = 5.5, 1.7 Hz, 2H), 3.33 (d, J = 8.4 Hz, 1H), 3.14 (dd, J = 14.0, 3.9 Hz, 1H), 2.94-2.81 (m, 2H), 2.74- 2.60 (m, 3H), 2.38 (d, J = 1.1 Hz, 3H), 2.01-1.86 (m, 1H), 1.85-1.71 (m, 2H), 1.62 (dd, J = 14.1, 11.4 Hz, 1H), 1.13 (d, J = 6.3 Hz, 3H); LCMS m/z 325.18 [M + H]⁺. |
| 503 | 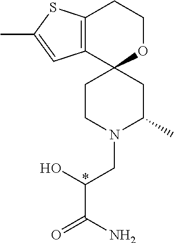 | 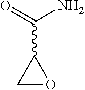 | S23² | ¹H NMR (300 MHz, Methanol-d₄) δ 6.48 (q, J = 1.1 Hz, 1H), 4.25 (dd, J = 9.2, 3.0 Hz, 1H), 3.90 (td, J = 5.5, 2.2 Hz, 2H), 3.33 (d, J = 8.4 Hz, 2H, 1H under MeOH), 3.07- 2.74 (m, 3H), 2.70 (dt, J = 6.9, 3.4 Hz, 2H), 2.39 (d, J = 1.1 Hz, 3H), 2.04-1.59 (m, 4H), 1.13 (d, J = 6.3 Hz, 3H). LCMS m/z 325.18 [M + H]⁺. |
| 504 | 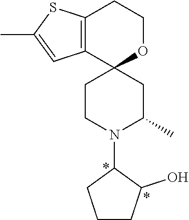 [SMILES: Cc1cc2c(s1)CCO[C@@]21CCN(C2CCCC2O)[C@@H](C)C1] | 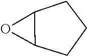 | S23³ | 1H NMR (300 MHz, Methanol-d₄) δ 8.53 (s, 1H), 6.49 (s, 1H), 4.48 (q, J = 7.1 Hz, 1H), 4.09-3.75 (m, 3H), 3.39 (t, J = 4.5 Hz, 2H), 2.74 (t, J = 5.5 Hz, 2H), 3.31 (2H under MeOH) 2.40 (d, J = 1.0 Hz, 3H), 2.23-1.97 (m, 6H), 1.96-1.57 (m, 2H), 1.46 (d, J = 6.4 Hz, 3H). LCMS m/z 322.27 [M + H]⁺. |
| 505 | 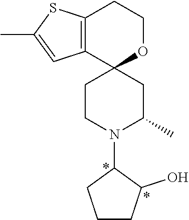 [SMILES: Cc1cc2c(s1)CCO[C@@]21CCN(C2CCCC2O)[C@@H](C)C1] | 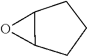 | S23³ | 1H NMR (300 MHz, Methanol-d₄) δ 8.53 (s, 1H), 6.52 (t, J = 1.2 Hz, 1H), 4.40 (q, J = 6.9 Hz, 1H), 4.04-3.85 (m, 2H), 3.75 (q, J = 8.2 Hz, 1H), 3.52 (h, J = 6.6 Hz, 1H), 3.31 (4H under MeOH) 2.74 (t, J = 5.4 Hz, 2H), 2.41 (d, J = 1.1 Hz, 3H), 2.26-1.57 (m, 7H), 1.40 (d, J = 6.5 Hz, 3H). LCMS m/z 322.27 [M + H]⁺. |
| 506 | 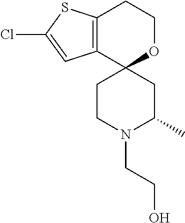 [SMILES: C[C@H]1C[C@@]2(CCN1CCO)OCCc1sc(Cl)cc12] | 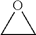 | S24⁴ | 1H NMR (300 MHz, Chloroform-d) δ 6.60 (s, 1H), 4.01-3.83 (m, 2H), 3.70 (ddd, J = 10.7, 9.2, 4.0 Hz, 1H), 3.56 (ddd, J = 10.7, 5.1, 3.6 Hz, 1H), 3.12 (ddd, J = 13.9, 9.2, 5.1 Hz, 1H), 2.86 (dt, J = 11.7, 3.5 Hz, 1H), 2.80-2.63 (m, 3H), 2.60-2.48 (m, 1H), 2.27 (dt, J = 13.0, 3.8 Hz, 1H), 1.92- 1.76 (m, 3H), 1.60 (t, J = 12.7 Hz, 1H), 1.09 (d, J = 6.2 Hz, 3H); LCMS m/z 302.12 [M + H]⁺. |
| 507 | 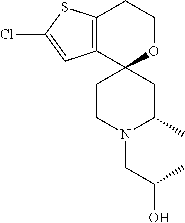 [SMILES: C[C@H](O)CN1CC[C@]2(C[C@@H]1C)OCCc1sc(Cl)cc12] | 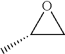 | S24^{d} | 1H NMR (300 MHz, Chloroform-d) δ 6.72 (s, 1H), 4.20 (ddt, J = 9.0, 6.3, 4.1 Hz, 1H), 3.90 (tdd, J = 11.4, 8.8, 4.9 Hz, 2H), 3.56 (d, J = 11.4 Hz, 1H), 3.24 (ddt, J = 33.7, 25.3, 10.6 Hz, 3H), 2.79-2.59 (m, 3H), 2.39-2.09 (m, 2H), 1.97 (ddt, J = 15.2, 12.0, 3.0 Hz, 2H), 1.38 (d, J = 6.4 Hz, 3H), 1.27 (d, J = 6.2 Hz, 3H); LCMS m/z 316.11 [M + H]⁺. |

Foornotes:
¹Oxirane-2-carboxamide (1.6 eq) and DIPEA (5.2 eq) were used. IPA was used instead of n-BuOH. The reaction was heated at 150° C. for 2 h. Purification was completed using silica gel chromatography (Gradient: 0 to 20% MeOH in DCM).
²The mixture was separated into constituent enantiomers by chiral SFC separation. Column: Daicel Chiralpak ® OJ-H, 10 × 250 mm; Mobile phase: 30% MeOH (containing 5 mM ammonia), 70% carbon dioxide. Flow: 15 mL/min.
³6-Oxabicyclo[3.1.0]hexane (4.2 eq) and DIPEA (8.5 eq) were used. The reaction was heated at 200° C. for 4 h.
⁴The corresponding oxirane (3.1 eq) and DIPEA (5.0 eq) were used. MeOH was used instead of n-BuOH. The reaction was heated at 90° C. for 2 h. Purification was completed using silic gel chromatography (Gradient: 0 to 15% MeOH in DCM).

### Compound 508

### (2S)-3-[(2′S,4R)-2-chloro-2′-methyl-spiro[6,7-dihydrothieno[3,2-c]pyran-4,4′-piperidine]-1′-yl]-2-hydroxy-propanamide (508)

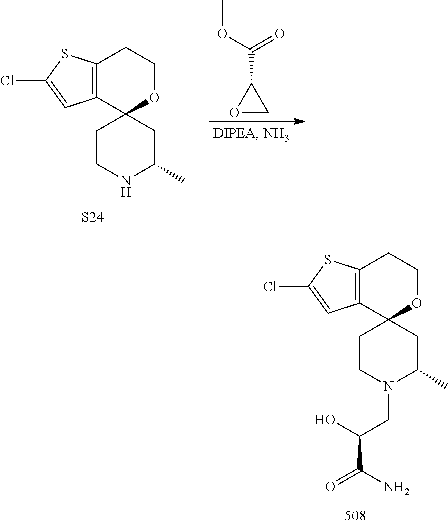

### Preparation of (2S)-3-[(2′S,4R)-2-chloro-2′-methyl-spiro[6,7-dihydrothieno[3,2-c]pyran-4,4′-piperidine]-1′-yl]-2-hydroxy-propanamide (508)

(2′S,4R)-2-chloro-2′-methyl-spiro[6,7-dihydrothieno[3,2-c]pyran-4,4′-piperidine]S24 (78 mg, 0.3026 mmol) and methyl (2S)-oxirane-2-carboxylate (260 μL, 2.970 mmol) were added to a microwave vial and added NH₃ (400 μL of 7 M, 2.800 mmol) in MeOH (1.5 mL). DIPEA (260 μL, 1.493 mmol) was added and reaction was heated at 120° C. for 3 h. An additional 200 μL (2S)-oxirane-2-carboxylate and NH₃ (400 μL of 7 M, 2.800 mmol) were added and the reaction was stirred at 120° C. for 7 h. The reaction mixture was concentrated in vacuo. Purification by reversed-phase HPLC. Method: C18 Waters Sunfire column (30×150 mm, 5 micron). Gradient: MeCN in H₂O with 0.1% trifluoroacetic acid. Further purification was performed using silica gel chromatography (Gradient: 1-16% MeOH in DCM) to provide (2S)-3-[(2′S,4R)-2-chloro-2′-methyl-spiro[6,7-dihydrothieno[3,2-c]pyran-4,4′-piperidine]-1′-yl]-2-hydroxy-propanamide (8.5 mg, 7%). ¹H NMR (300 MHz, Chloroform-d) δ 11.37 (s, 1H), 8.30 (s, 1H), 7.20 (s, 1H), 6.68 (s, 1H), 5.58 (s, 1H), 4.69 (d, J=9.8 Hz, 1H), 4.07-3.84 (m, 2H), 3.65 (s, 1H), 3.49 (d, J=11.8 Hz, 1H), 3.29 (s, OH), 2.92 (t, J=11.9 Hz, 1H), 2.76 (q, J=5.5 Hz, 2H), 2.37 (dd, J=32.2, 15.7 Hz, 2H), 2.00 (dd, J=14.9, 8.1 Hz, 2H), 1.49 (d, J=6.4 Hz, 3H). LCMS m/z 345.21 [M+H]⁺.

### Compound 509

### (2R)-3-[(2′S,4R)-2-chloro-2′-methyl-spiro[6,7-dihydrothieno[3,2-c]pyran-4,4′-piperidine]-1′-yl]-2-hydroxy-propanamide

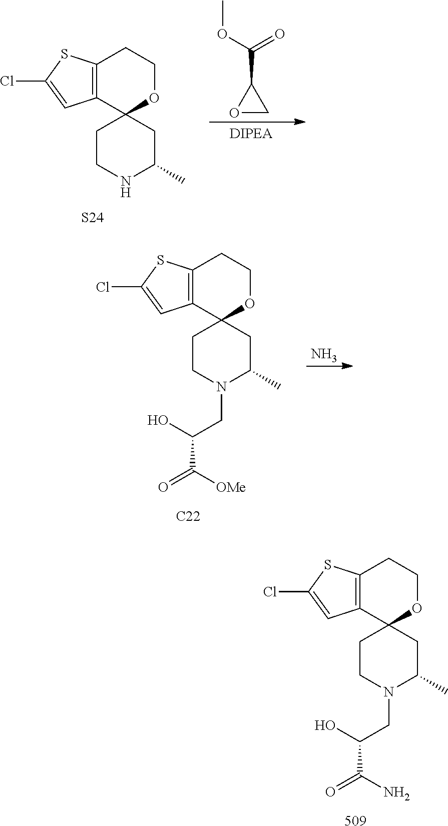

### Step 1. Synthesis of methyl (2R)-3-[(2′S,4R)-2-chloro-2′-methyl-spiro[6,7-dihydrothieno[3,2-c]pyran-4,4′-piperidine]-1′-yl]-2-hydroxy-propanoate (C22)

Methyl (2R)-3-[(2′S,4R)-2-chloro-2′-methyl-spiro[6,7-dihydrothieno[3,2-c]pyran-4,4′-piperidine]-1′-yl]-2-hydroxy-propanoate C22 was prepared following epoxide opening method as describe for 508 starting with S24 and (2R)-oxirane-2-carboxylate.

### Step 2. Synthesis of (2R)-3-[(2′S,4R)-2-chloro-2′-methyl-spiro[6,7-dihydrothieno[3,2-c]pyran-4,4′-piperidine]-1′-yl]-2-hydroxy-propanamide (509)

Methyl (2R)-3-[(2′S,4R)-2-chloro-2′-methyl-spiro[6,7-dihydrothieno[3,2-c]pyran-4,4′-piperidine]-1′-yl]-2-hydroxy-propanoate (66 mg, 0.1834 mmol) was added to a microwave vial and added NH₃ (1.3 mL of 7 M, 9.100 mmol) in MeOH (100 μL). The reaction mixture was stirred at 50° C. overnight. The reaction mixture was concentrated in vacuo and purified via silica gel chromatography (Gradient: 0-12% MeOH in DCM) to provide (2R)-3-[(2′S,4R)-2-chloro-2′-methyl-spiro[6,7-dihydrothieno[3,2-c]pyran-4,4′-piperidine]-1′-yl]-2-hydroxy-propanamide 509 (21.1 mg, 31%). ¹H NMR (300 MHz, Chloroform-d) δ 7.01 (d, J=3.9 Hz, 1H), 6.59 (s, 1H), 5.92 (s, 1H), 4.19 (dd, J=9.9, 4.9 Hz, 1H), 3.89 (h, J=6.1 Hz, 2H), 3.15-2.86 (m, 3H), 2.80-2.59 (m, 3H), 1.84 (dt, J=14.9, 4.7 Hz, 3H), 1.65 (dd, J=14.2, 11.4 Hz, 1H), 1.44 (dd, J=17.0, 7.0 Hz, 1H), 1.10 (d, J=6.2 Hz, 3H).

### Compound 510

### (2′S)-2,2′-dimethyl-1′-[[1-(2-methylsulfonylethyl)triazol-4-yl]methyl]spiro[6,7-dihydrothieno[3,2-c]pyran-4,4′-piperidine]

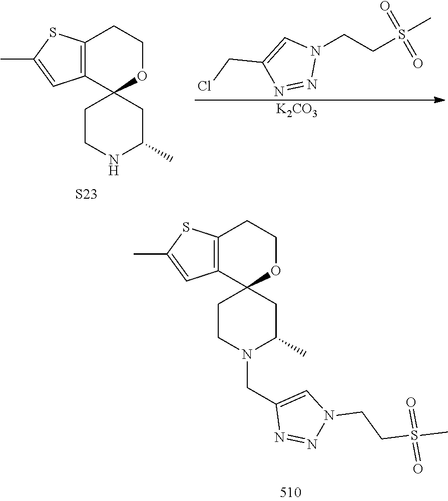

### Preparation of (2′S)-2,2′-dimethyl-1′-[[1-(2-methylsulfonylethyl)triazol-4-yl]methyl]spiro[6,7-dihydrothieno[3,2-c]pyran-4,4′-piperidine](510)

In a reaction vial was added (2′S)-2,2′-dimethylspiro[6,7-dihydrothieno[3,2-c]pyran-4,4′-piperidine](trifluoromethanesulfonate) S23 (260 mg, 0.6148 mmol), 4-(chloromethyl)-1-(2-methylsulfonylethyl)triazole (151 mg, 0.6751 mmol), K₂CO₃ (255 mg, 1.845 mmol), and DMF (1.5 mL). The reaction mixture was heated to 60° C. and stirred for 6 h, then allowed to cool to rt and stirred overnight. The reaction was diluted with DCM, washed with NaHCO₃, and extracted with DCM. The combined organic layers were concentrated in vacuo, then purified by silica gel chromatography (Gradient: 0-10% MeOH in DCM) to provide (2′S)-2,2′-dimethyl-1′-[[1-(2-methylsulfonylethyl)triazol-4-yl]methyl]spiro[6,7-dihydrothieno[3,2-c]pyran-4,4′-piperidine]510 (157.4 mg, 57%). ¹H NMR (300 MHz, Chloroform-d) δ 7.81 (s, 1H), 6.45 (d, J=1.3 Hz, 1H), 4.87 (t, J=6.4 Hz, 2H), 4.17 (d, J=14.7 Hz, 1H), 3.96 (d, J=14.5 Hz, 1H), 3.89-3.78 (m, 2H), 3.73 (t, J=6.4 Hz, 2H), 2.71 (d, J=11.4 Hz, 8H), 2.39 (d, J=1.0 Hz, 3H), 2.00 (s, 1H), 1.85 (d, J=10.0 Hz, 3H), 1.31 (d, J=6.3 Hz, 3H). LCMS m/z 425.32 [M+H]⁺.

### Compounds 511-514

Compounds 511-514 (see Table 15) were prepared in a single step from the appropriate intermediate piperidine and alkyl halide using the alkylation method as for compound 510. Alkyl halides were obtained from commercial sources or described previously. Any modifications to methods are noted in Table 15 and accompanying footnotes.

TABLE 15
Structure and physicochemical data for compounds 511-514

| | | | Starting | |
|---|---|---|---|---|
| Compd | Structure | Alkyl halide material | [M + H]⁺ | ¹H NMR; LCMS m/z |
| 511 | 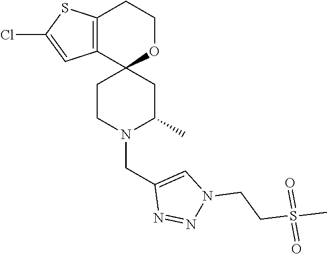 [SMILES: C[C@H]1C[C@@]2(CCN1Cc1cn(CCS(C)(=O)=O)nn1)OCCc1sc(Cl)cc12] | 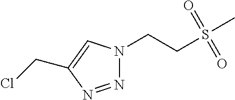 | S24¹ | 1H NMR (300 MHz, Methanol-d₄) δ 8.11 (s, 1H), 6.73 (s, 1H), 4.94 (dd, J = 7.0, 6.1 Hz, 2H), 4.18-4.01 (m, 2H), 3.87- 3.79 (m, 4H), 2.91 (d, J = 0.7 Hz, 3H), 2.80 (dd, J = 22.9, 9.4 Hz, 3H), 2.67 (td, J = 5.4, 1.7 Hz, 2H), 1.91-1.83 (m, 3H), 1.70 (dd, J = 14.3, 11.5 Hz, 1H), 1.31 (d, J = 6.3 Hz, 3H); LCMS m/z 445.21 [M + H]⁺. |
| 512 | 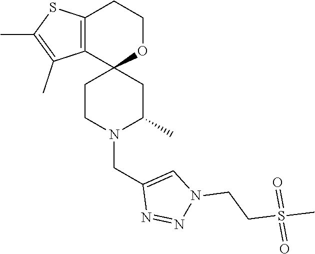 | 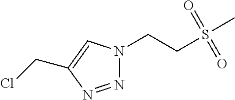 | S19² | LCMS m/z 439.13 [M + H]⁺. |
| 513 | 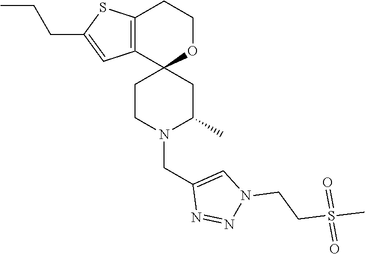 [SMILES: CCCc1cc2c(s1)CCO[C@@]21CCN(Cc2cn(CCS(C)(=O)=O)nn2)[C@@H](C)C1] | 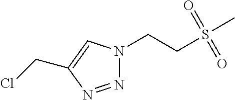 | S20² | LCMS m/z 453.17 [M + H ]⁺. |
| 514 | 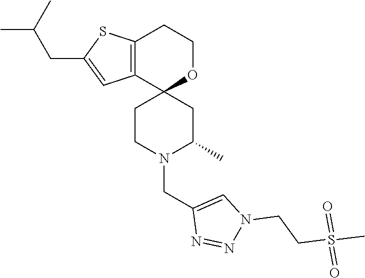 [SMILES: CC(C)Cc1cc2c(s1)CCO[C@@]21CCN(Cc2cn(CCS(C)(=O)=O)nn2)[C@@H](C)C1] | 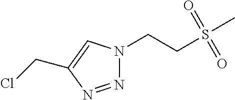 | S21² | LCMS m/z 467.18 [M + H]⁺. |

Foornotes:
¹Purification by silica gel chromatography (Gradient: 10-100% EtOAc in heptane).
²Purification by reversed-phase HPLC. Method: C18 Waters Sunfire column (30 × 150 mm, 5 micron). Gradient: MeCN in H₂O with 0.1% trifluoroacetic acid.

### Compound 515

### 2-[[5-[[(2′S,4R)-2-chloro-2′-methyl-spiro[6,7-dihydrothieno[3,2-c]pyran-4,4′-piperidine]-1′-yl]methyl]pyrimidin-2-yl]amino]-2-methyl-propan-1-ol

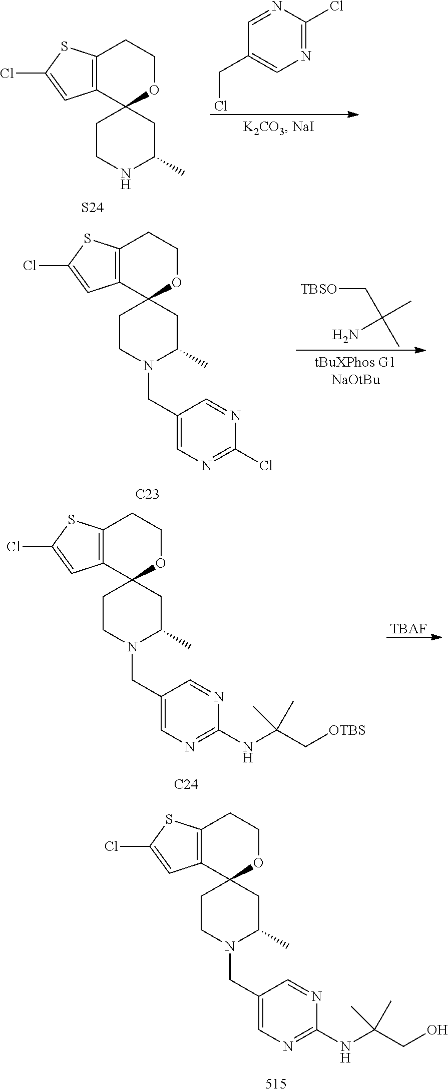

### Step 1. Synthesis of (2′S,4R)-2-chloro-1′-[(2-chloropyrimidin-5-yl)methyl]-2′-methyl-spiro[6,7-dihydrothieno[3,2-c]pyran-4,4′-piperidine](C23)

A microwave vial was charged with (2′S,4R)-2-chloro-2′-methyl-spiro[6,7-dihydrothieno[3,2-c]pyran-4,4′-piperidine]S24 (trifluoromethanesulfonate) (250 mg, 0.6011 mmol), 2-chloro-5-(chloromethyl)pyrimidine (118 mg, 0.7239 mmol), K₂CO₃ (965 mg, 1.930 mmol), and NaI (91 mg, 0.6071 mmol), followed by THF (2.2 mL) and DMF (250 μL). The resulting mixture was heated at 40° C. overnight, then partitioned between EtOAc and water. The organic layer was washed with brine, dried over anhydrous sodium sulfate, filtered, and concentrated in vacuo. Purification by silica gel chromatography (Gradient: 10-100% EtOAc in heptane) provided (2′S,4R)-2-chloro-1′-[(2-chloropyrimidin-5-yl)methyl]-2′-methyl-spiro[6,7-dihydrothieno[3,2-c]pyran-4,4′-piperidine] C23 (143.7 mg, 59%). LCMS m/z 384.01 [M+H]⁺.

### Step 2. Synthesis of N-[2-[tert-butyl(dimethyl)silyl]oxy-1,1-dimethyl-ethyl]-5-[[(2′S,4R)-2-chloro-2′-methyl-spiro[6,7-dihydrothieno[3,2-c]pyran-4,4′-piperidine]-1′-yl]methyl]pyrimidin-2-amine (C24)

A mixture of (2′S,4R)-2-chloro-1′-[(2-chloropyrimidin-5-yl)methyl]-2′-methyl-spiro[6,7-dihydrothieno[3,2-c]pyran-4,4′-piperidine] C23 (143.7 mg, 0.3519 mmol), 1-[tert-butyl(dimethyl)silyl]oxy-2-methyl-propan-2-amine (108 mg, 0.5310 mmol), tBuXPhos Pd G1 (15 mg, 0.02303 mmol), in t-BuOH (3.5 mL) was degassed under nitrogen for 10 min, followed by the addition of NaOtBu (388 μL of 2 M, 0.7760 mmol). The resulting mixture was sealed and heated at 60° C. for 45 min. Partitioned between EtOAc and water. The combined organics were dried over anhydrous sodium sulfate, filtered, and concentrated in vacuo to afford N-[2-[tert-butyl(dimethyl)silyl]oxy-1,1-dimethyl-ethyl]-5-[[(2′S,4R)-2-chloro-2′-methyl-spiro[6,7-dihydrothieno[3,2-c]pyran-4,4′-piperidine]-1′-yl]methyl]pyrimidin-2-amine. LCMS m/z 551.28 [M+H]⁺.

### Step 3. Synthesis of 2-[[5-[[(2′S,4R)-2-chloro-2′-methyl-spiro[6,7-dihydrothieno[3,2-c]pyran-4,4′-piperidine]-1′-yl]methyl]pyrimidin-2-yl]amino]-2-methyl-propan-1-ol (515)

The crude product from step 2 was dissolved in THF (3 mL) and treated with TBAF (1.8 mL of 1 M in THF, 1.800 mmol). The reaction was stirred at rt for 2 h, then partitioned between EtOAc and water. The combined organics were dried over anhydrous sodium sulfate, filtered, and concentrated in vacuo. Purification by HPLC: 10-90% ACN in Water (HCl modifier) to afford 2-[[5-[[(2′S,4R)-2-chloro-2′-methyl-spiro[6,7-dihydrothieno[3,2-c]pyran-4,4′-piperidine]-1′-yl]methyl]pyrimidin-2-yl]amino]-2-methyl-propan-1-ol (hydrochloride salt) (6.3 mg, 4%). ¹H NMR (300 MHz, Methanol-d₄) δ 8.43 (s, 2H), 6.81 (s, 1H), 4.56 (s, 1H), 3.96 (h, J=6.1 Hz, 3H), 3.69 (s, 2H), 3.56 (s, 1H), 3.18 (s, 2H), 2.74 (td, J=5.3, 1.9 Hz, 2H), 2.09 (dd, J=23.2, 8.1 Hz, 4H), 1.56 (s, 3H), 1.39 (s, 6H). LCMS m/z 437.11 [M+H]⁺.

### Compound 516

### (2′S,4S)-2,2′-dimethyl-1′-[[1-(2-methylsulfonylethyl)pyrazol-4-yl]methyl]spiro[6,7-dihydrothieno[3,2-c]pyran-4,4′-piperidine](516)

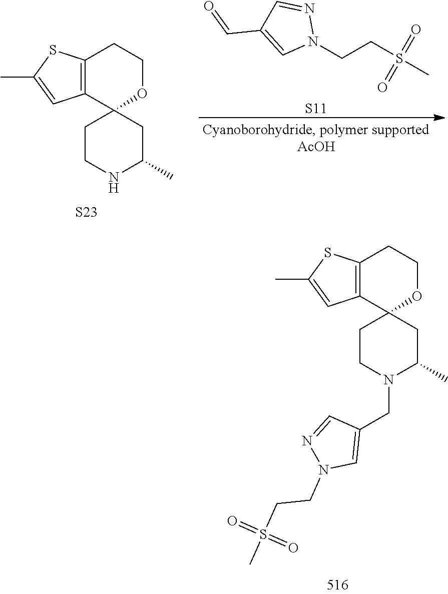

### Preparation of (2′S,4S)-2,2′-dimethyl-1′-[[1-(2-methylsulfonylethyl)pyrazol-4-yl]methyl]spiro[6,7-dihydrothieno[3,2-c]pyran-4,4′-piperidine](516)

(2′S,4S)-2,2′-dimethylspiro[6,7-dihydrothieno[3,2-c]pyran-4,4′-piperidine]S23 (27.6 mg, 0.1163 mmol) and 1-(2-methylsulfonylethyl)pyrazole-4-carbaldehyde (41 mg, 0.2027 mmol) were dissolved in DCM (750 μL) and acetic acid (40 μL, 0.7034 mmol) was added to the solution followed by cyanoborohydride, polymer supported (186 mg of 2 mmol/g, 0.3720 mmol). The solution was heated to 90° C. in a microwave reactor for 95 min. An additional portion of cyanoborohydride, polymer supported (67 mg, 0.5930 mmol) was added and the reaction was stirred overnight at rt. The suspension was stirred in 1.5 mL of MeOH for 10 min before filtering off the resin. The solvent was removed, and the residue was dissolved into water (2 mL) and DCM (2 mL). The pH of the aqueous layer was adjusted with 2M NaOH to a pH >10. The phases were separated through a phase separator and the aqueous layer was extracted with DCM (2×10 mL) and the combined organics were concentrated in vacuo. The crude residue was purified by silica gel chromatography (Gradient: 0-20% MeOH in DCM) to yield (2′S,4S)-2,2′-dimethyl-1′-[[1-(2-methylsulfonylethyl)pyrazol-4-yl]methyl]spiro[6,7-dihydrothieno[3,2-c]pyran-4,4′-piperidine](32.1 mg, 62%). ¹H NMR (300 MHz, Chloroform-d) δ 7.54 (s, 1H), 7.49 (s, 1H), 6.46 (d, J=1.3 Hz, 1H), 4.58 (t, J=6.8 Hz, 2H), 4.00-3.80 (m, 2H), 3.64 (t, J=6.1 Hz, 2H), 3.55 (s, 2H), 3.06 (q, J=5.9, 5.3 Hz, 1H), 2.91-2.58 (m, 3H), 2.56-2.43 (m, 1H), 2.47 (s, 3H), 2.40 (s, 3H), 2.03-1.76 (m, 4H), 1.17 (d, J=6.8 Hz, 3H).

### Compounds 517-518

Compounds 517-518 (see Table 16) were prepared in a single step from intermediate S22 and S24 using reductive amination step as for compound 516. Aldehydes were described previously. Any modifications to methods are noted in Table 16 and accompanying footnotes.

TABLE 16
Structure and physicochemical data for compounds 517-518

| | | Aldehyde | Starting | ¹H NMR; LCMS m/z |
|---|---|---|---|---|
| Compd | Product | Reagent | Material | [M + H]⁺ |
| 517 | 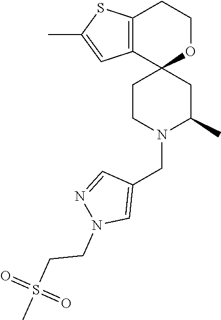 [SMILES: Cc1cc2c(s1)CCO[C@@]21CCN(Cc2cnn(CCS(C)(=O)=O)c2)[C@H](C)C1] | 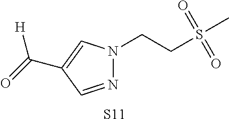 | 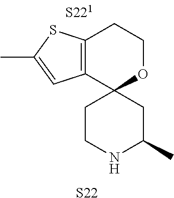 | ¹H NMR (300 MHz, Methanol-d₄) δ 7.74 (s, H), 7.58 (d, J = 0.7 Hz, 1H), 6.45 (d, J = 1.3 Hz, 1H), 4.62 (t, J = 6.5 Hz, 2H), 3.89 (dd, J = 5.9, 5.0 Hz, 2H), 3.68 (t, J = 6.4 Hz, 2H), 3.64 (d, J = 1.3 Hz, 2H), 3.08 (dq, J = 10.4, 6.5 Hz, 1H), 2.97- 2.79 (m, 1H), 2.76 (s, 3H), 2.74-2.54 (m, 3H), 2.36 (d, J = 1.0 Hz, 3H), 2.06-1.69 (m, 4H), 1.24 (d, J = 6.8 Hz, 3H). |
| 518 | 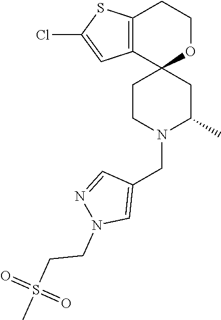 [SMILES: C[C@H]1C[C@@]2(CCN1Cc1cnn(CCS(C)(=O)=O)c1)OCCc1sc(Cl)cc12] | 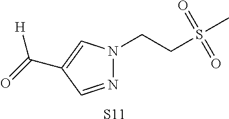 | S24² | ¹H NMR (300 MHz, Methanol-d₄) δ 8.02 (s, 1H), 7.74 (d, J = 0.7 Hz, 1H), 6.76 (d, J = 1.1 Hz, 1H), 5.49 (s, 1H), 4.76- 4.66 (m, 2H), 4.52 (d, J = 14.1 Hz, 1H), 4.28 (d, J = 14.1 Hz, 1H), 3.97-3.84 (m, 2H), 3.72 (t, J = 6.2 Hz, 2H), 3.53-3.46 (m, 1H), 2.91-2.82 (m, 3H), 2.79-2.69 (m, 2H), 2.25- 1.91 (m, 4H), 1.54 (d, J = 6.5 Hz, 3H), 1.17 (t, J = 7.0 Hz, 1H); LCMS m/z 443.99 [M + H]⁺. |

Foornotes:
¹Different reagent amounts were used: 1-(2-methylsulfonylethyl)pyrazole-4-carbaldehyde (2.2 eq), acetic acid (7.0 eq), cyanoborohydride, polymer supported (4.7 eq).
²Different reagent amounts were used: 1-(2-methylsulfonylethyl)pyrazole-4-carbaldehyde (1.5 eq), acetic acid (5.0 eq), cyanoborohydride, polymer supported (2.8 eq). The reaction was performed at 110° C. for 1 h.

### Compound 519

### (2′S,4R)-2,2′,3-trimethyl-1′-[(1-methylpyrazol-4-yl)methyl]spiro[6,7-dihydrothieno[3,2-c]pyran-4,4′-piperidine](519)

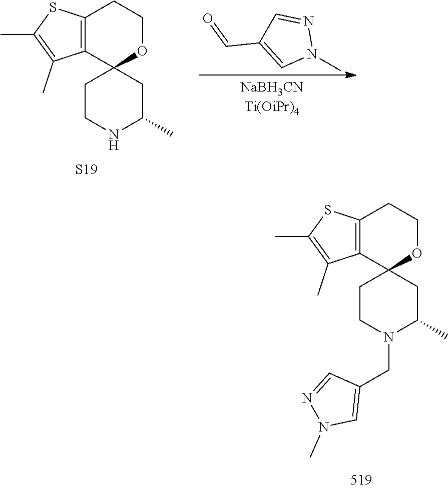

### Preparation of (2′S,4R)-2,2′,3-trimethyl-1′-[(1-methylpyrazol-4-yl)methyl]spiro[6,7-dihydrothieno[3,2-c]pyran-4,4′-piperidine](519)

To a stirred solution of (2′S,4R)-2,2′,3-trimethylspiro[6,7-dihydrothieno[3,2-c]pyran-4,4′-piperidine]S19 (150 mg, 0.5967 mmol) in MeOH (2 mL) was added 1-methylpyrazole-4-carbaldehyde (131.41 mg, 1.1934 mmol) and titanium isopropoxide (508.77 mg, 0.5283 mL, 1.7901 mmol) and stirred for 2 h at 50° C. Sodium cyanoborohydride (112.49 mg, 1.7901 mmol) was added to the reaction mixture and stirred for 24 h at 50° C. The reaction mixture was concentrated in vacuo and washed with water (4 ml) and extracted with EtOAc (3×5 ml). Organic layer were dried over sodium sulfate and concentrated in vacuo and purified by reverse phase HPLC chromatography. Method: YMC Triart Actus C18 (250×20 mm, 5 micron). Gradient: MeCN in H₂O with 20 mM ammonium bicarbonate. Product was isolated as (2′S,4R)-2,2′,3-trimethyl-1′-[(1-methylpyrazol-4-yl)methyl]spiro[6,7-dihydrothieno[3,2-c]pyran-4,4′-piperidine](56.3 mg, 27%). 1H NMR (400 MHz, DMSO-d₆) δ 7.57 (s, 1H), 7.29 (s, 1H), 3.80 (bs, 3H), 3.77-3.65 (m, 3H), 3.45 (d, J=14.52 Hz, 1H), 2.60 (bs, 2H), 2.43-2.32 (m, 3H), 2.21 (s, 3H), 2.09 (s, 3H), 2.02-1.97 (m, 1H), 1.75 (t, J=11.6 Hz, 1H), 1.59 (d, J=13.8 Hz, 2H), 1.09 (d, J=5.8 Hz, 3H). LCMS m/z 346.3 [M+H]⁺.

### Compounds 520-521

Compounds 520-521 (see Table 17) were prepared in a single step from the appropriate piperidine chosen from intermediates S20 or S21 using reductive animation step as for compound 519. Aldehydes were obtained from commercial sources. Any modifications to methods are noted in Table 17 and accompanying footnotes.

TABLE 17
Structure and physicochemical data for compounds 520-521

| | | Aldehyde | |
|---|---|---|---|
| Compound | Product | Reagent | ¹H NMR; LCMS m/z [M + H]⁺ |
| 520 | 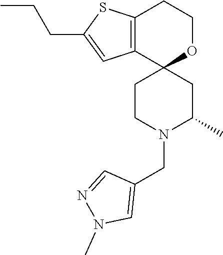 [SMILES: CCCc1cc2c(s1)CCO[C@@]21CCN(Cc2cnn(C)c2)[C@@H](C)C1] | 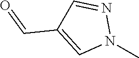 | ¹H NMR (400 MHz, DMSO-d₆) δ 7.55 (s, 1H), 7.27 (s, 1H), 6.57 (s, 1H), 3.79-3.65 (m, 6H), 3.39-3.31 (m, 1H), 3.30 (s, 3H), 2.66-2.61 (m, 4H), 2.40-2.32 (m, 2H), 1.73- 1.65 (m, 2H), 1.59-1.52 (m, 2H), 1.08 (d, J = 6.12 Hz, 3H), 0.90 (t, J = 7.28 Hz, 3H); LCMS m/z 360.3 [M + H]⁺. |
| 521 | 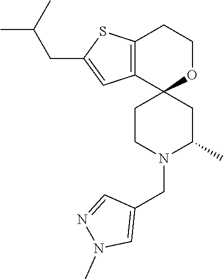 [SMILES: CC(C)Cc1cc2c(s1)CCO[C@@]21CCN(Cc2cnn(C)c2)[C@@H](C)C1] | 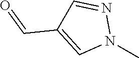 | ¹H NMR (400 MHz, DMSO-d6) δ: 7.55 (s, 1H), 7.28 (s, 1H), 6.54 (s, 1H), 3.79-3.74 (m, 5H), 3.67 (d, J = 14.0 Hz, 1H), 3.41 (d, J = 14.0 Hz, 1H), 2.61 (bs, 2H), 2.54 (d, J = 6.92 Hz, 2H), 2.40-2.35 (m, 2H), 1.77-1.66 (m, 4H), 1.55-1.52 (m, 2H), 1.08 (d, J = 5.88 Hz, 3H), 0.88 (d, J = 6.56 Hz, 6H); LCMS m/z 374.3 [M + H]⁺. |

### Preparation S25

### tert-butyl (2′S,4R)-2-bromo-2′-methyl-spiro[6,7-dihydrothieno[3,2-c]pyran-4,4′-piperidine]-1′-carboxylate (S25)

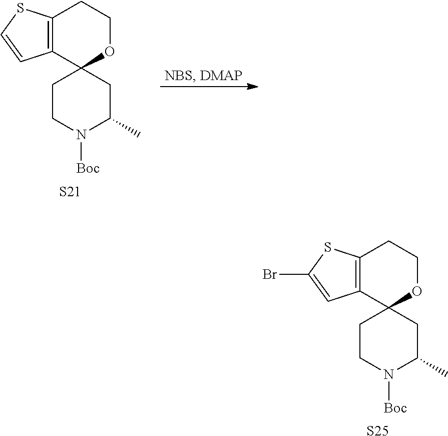

### Preparation of tert-butyl (2′S,4R)-2-bromo-2′-methyl-spiro[6,7-dihydrothieno[3,2-c]pyran-4,4′-piperidine]-1′-carboxylate (S25)

To a stirring solution of tert-butyl (2′S,4R)-2′-methylspiro[6,7-dihydrothieno[3,2-c]pyran-4,4′-piperidine]-1′-carboxylate S21 (220 mg, 564.54 mol) in ACN (4 mL) was added NBS (100 mg, 561.85 mol) and DMAP (0.6 mg, 4.9113 mol) at rt. Then reaction mixture was heated to 65° C. and stirred for overnight. The reaction mixture was diluted with 1 N NaOH (25 mL), extracted with EtOAc (2×50 ml). The organic layer was washed with saturated aqueous sodium thiosulfate solution (50 ml), then washed with brine solution (50 ml), dried over Na₂SO₄. The organic layer was concentrated in vacuo to provide the crude material. The crude compound was purified by silica gel chromatography (Gradient: 10-15% EtOAc in petroleum ether) to yield tert-butyl (2′S,4R)-2-bromo-2′-methyl-spiro[6,7-dihydrothieno[3,2-c]pyran-4,4′-piperidine]-1′-carboxylate S25 (140 mg, 59%). ¹H NMR (400 MHz, Chloroform-d) δ 6.55 (s, 1H), 5.30 (s, 1H), 3.98-3.88 (m, 1H), 3.87-3.85 (m, 1H), 3.74-3.71 (m, 1H), 3.32-3.28 (m, 1H), 2.71-2.66 (m, 2H), 1.96-1.91 (m, 2H), 1.76-1.64 (m, 2H), 1.48 (s, 9H), 1.24 (d, J=6.4 Hz, 3H). LCMS m/z 404.24 [M+H]⁺.

### Compound 522

### (2′S,4R)-2-bromo-2′-methyl-1′-[[1-(2-methylsulfonylethyl)triazol-4-yl]methyl]spiro[6,7-dihydrothieno[3,2-c]pyran-4,4′-piperidine](522)

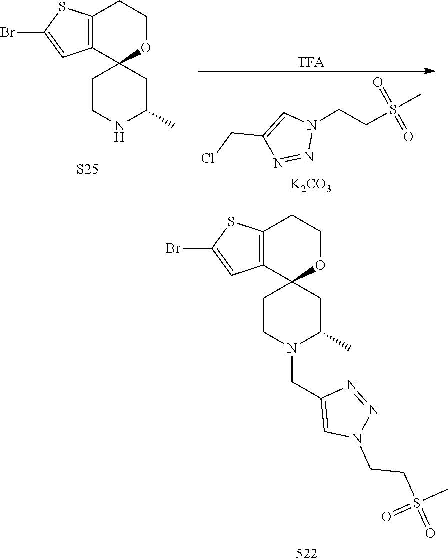

### Preparation of (2′S,4R)-2-bromo-2′-methyl-1′-[[1-(2-methylsulfonylethyl)triazol-4-yl]methyl]spiro[6,7-dihydrothieno[3,2-c]pyran-4,4′-piperidine](522)

To a mixture of tert-butyl (2′S,4R)-2-bromo-2′-methyl-spiro[6,7-dihydrothieno[3,2-c]pyran-4,4′-piperidine]-1′-carboxylate S25 (50 mg, 0.1156 mmol) in DCM (1 mL) was added trifluoroacetic acid (200 μL, 2.596 mmol). After stirring 5 min, the mixture was dried, and redissolved in acetonitrile (2 mL) and to it was added 4-(chloromethyl)-1-(2-methylsulfonylethyl)triazole (Hydrochloride salt) (45 mg, 0.1730 mmol) and potassium carbonate (50 mg, 0.3618 mmol). The mixture was stirred at 70° C. for 3 h. The mixture was cooled to rt, diluted with water (3 mL) and EtOAc (5 mL), the layers were mixed and the aqueous layer was removed. The organic layer was washed with sat. brine, dried with magnesium sulfate, filtered and concentrated. The crude material was purified by silica gel chromatography (Gradient: 0-10% MeOH in DCM) to yield (2′S,4R)-2-bromo-2′-methyl-1′-[[1-(2-methylsulfonylethyl)triazol-4-yl]methyl]spiro[6,7-dihydrothieno[3,2-c]pyran-4,4′-piperidine]522 (33 mg, 58%)¹H NMR (400 MHz, Chloroform-d) δ 7.55 (s, 1H), 6.65 (s, 1H), 4.83-4.75 (m, 2H), 4.00-3.71 (m, 5H), 3.66 (t, J=6.3 Hz, 2H), 2.68-2.55 (m, 6H), 2.48 (s, 2H), 1.80-1.71 (m, 3H), 1.58 (d, J=12.7 Hz, 1H), 1.16 (d, J=6.1 Hz, 3H). LCMS m/z 488.97 [M+H]⁺.

### Compound 523

### (2′S,4R)-2-(2,2-difluoroethyl)-2′-methyl-1′-[[1-(2-methylsulfonylethyl)pyrazol-4-yl]methyl]spiro[6,7-dihydrothieno[3,2-c]pyran-4,4′-piperidine](523)

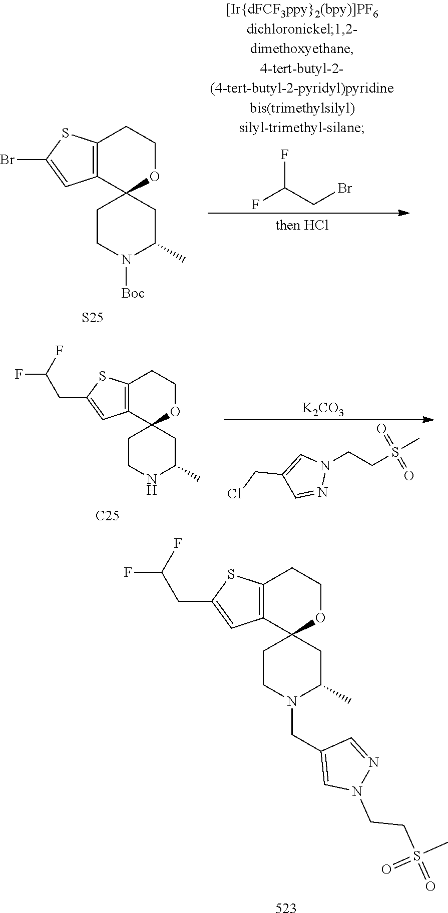

### Step 1. Synthesis of (2′S,4R)-2-(2,2-difluoroethyl)-2′-methyl-spiro[6,7-dihydrothieno[3,2-c]pyran-4,4′-piperidine](C25)

[Ir{dFCFppy}₂(bpy)]PF₆ (4 mg, 0.003962 mmol), 4-tert-butyl-2-(4-tert-butyl-2-pyridyl)pyridine (10 mg, 0.03726 mmol) and dichloronickel; 1,2-dimethoxyethane (10 mg, 0.04551 mmol) were added to a 1 dram vial, which was evacuated and refilled with nitrogen three times. bis(trimethylsilyl)silyl-trimethyl-silane (190 μL, 0.6159 mmol), 2,6-dimethylpyridine (90 μL, 0.7769 mmol), 2-bromo-1,1-difluoro-ethane (50 μL) and tert-butyl (2′S,4R)-2-bromo-2′-methyl-spiro[6,7-dihydrothieno[3,2-c]pyran-4,4′-piperidine]-1′-carboxylate (150 mg, 0.3728 mmol) were dissolved in 1,2-dimethoxyethane (2 mL) under nitrogen. Reaction was irradiated in a Merck Photoreactor at 100% LED power, 4700 RPM fan for 2 h. Reaction was diluted with 3 mL EtOAc and 1 mL water. Reaction was extracted and the organic layer was dried before evaporating off the volatiles. The crude residue was dissolved in HCl (1000 μL of 4 M, 4.000 mmol) and stirred for 1 h before removing the volatiles. Purification by reversed-phase HPLC. Method: C18 Waters Sunfire column (30×150 mm, 5 micron). Gradient: MeCN in H₂O with 0.1% trifluoroacetic acid. yielded (2′S,4R)-2-(2,2-difluoroethyl)-2′-methyl-spiro[6,7-dihydrothieno[3,2-c]pyran-4,4′-piperidine] C25 (Trifluoroacetate salt) (33.2 mg, 22%). 1H NMR (400 MHz, DMSO-d6) δ 8.91 (s, 2H), 6.66 (s, 1H), 6.40-6.02 (m, 1H), 3.89 (hept, J=6.0, 5.5 Hz, 2H), 3.70-3.37 (m, 3H), 3.12 (dd, J=54.9, 12.4 Hz, 2H), 2.74 (t, J=5.4 Hz, 2H), 2.10-1.83 (m, 4H), 1.24 (d, J=6.4 Hz, 3H). LCMS m/z 288.48 [M+H]⁺.

### Step 2. Synthesis of (2′S,4R)-2-(2,2-difluoroethyl)-2′-methyl-1′-[[1-(2-methylsulfonylethyl)pyrazol-4-yl]methyl]spiro[6,7-dihydrothieno[3,2-c]pyran-4,4′-piperidine](523)

(2′S,4R)-2-(2,2-difluoroethyl)-2′-methyl-spiro[6,7-dihydrothieno[3,2-c]pyran-4,4′-piperidine] C25 (30 mg) was dissolved in ACN (1 mL) and 4-(chloromethyl)-1-(2-methylsulfonylethyl)pyrazole (Hydrochloride salt) (17 mg, 0.06560 mmol) and K₂CO₃ (100 mg, 0.7236 mmol) were added. Heated to 65° C. overnight under nitrogen. The reaction was then diluted with water and DCM before separating the layers and extracting the water layer with DCM (×2) on a phase separator. The organic layer was concentrated in vacuo and purified with silica gel chromatography (Gradient: 0-20% MeOH in DCM) to yield (2′S,4R)-2-(2,2-difluoroethyl)-2′-methyl-1′-[[1-(2-methylsulfonylethyl)pyrazol-4-yl]methyl]spiro[6,7-dihydrothieno[3,2-c]pyran-4,4′-piperidine](6.1 mg, 19%). ¹H NMR (400 MHz, DMSO-d₆) δ 7.71 (s, 1H), 7.39 (s, 1H), 6.77 (s, 1H), 6.19 (tt, J=56.2, 4.2 Hz, 1H), 4.51 (t, J=6.8 Hz, 2H), 3.82-3.65 (m, 5H), 3.51-3.26 (m, 4H), 2.77 (s, 3H), 2.65 (s, 2H), 2.44-2.25 (m, 2H), 1.76-1.46 (m, 4H), 1.09 (d, J=6.1 Hz, 3H). LCMS m/z 474.02 [M+H]⁺.

### Compound 524

### (2′S,4R)-2-(difluoromethyl)-2′-methyl-1′-[[1-(2-methylsulfonylethyl)triazol-4-yl]methyl]spiro[6,7-dihydrothieno[3,2-c]pyran-4,4′-piperidine]

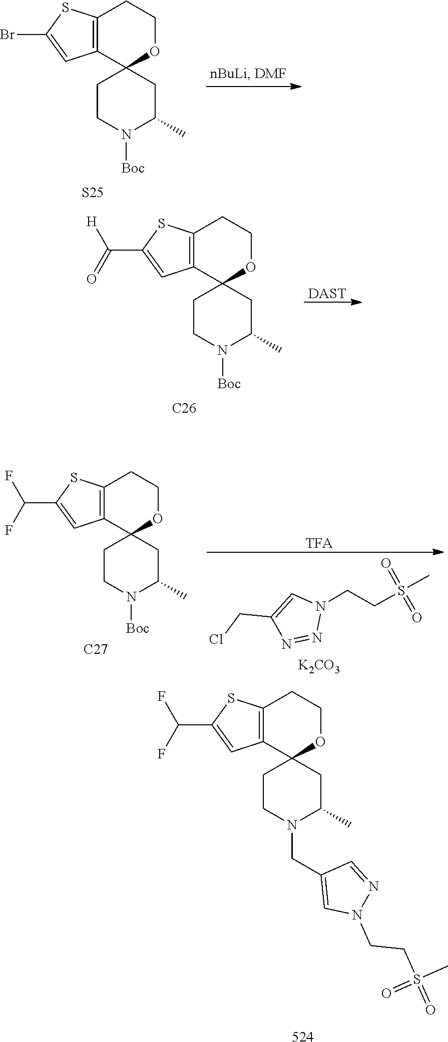

### Step 1. Synthesis of tert-butyl (2′S,4R)-2-formyl-2′-methyl-spiro[6,7-dihydrothieno[3,2-c]pyran-4,4′-piperidine]-1′-carboxylate (C26)

A solution of tert-butyl (2′S,4R)-2-bromo-2′-methyl-spiro[6,7-dihydrothieno[3,2-c]pyran-4,4′-piperidine]-1′-carboxylate S25 (50 mg, 0.1243 mmol) in THF (1 mL) was cooled to −78° C. At this time, butyllithium (50 μL of 2.5 M, 0.1250 mmol) in hexane was added, dropwise. After stirring at this temperature for 60 min, DMF (10 μL, 0.1291 mmol) was added. After another 50 min, the mixture was warmed slowly to rt. The reaction was quenched with sat. ammonium chloride, diluted with 1 mL of water and 5 mL EtOAc. The layers were mixed and separated, and the organic layer was washed with sat. brine. The organic layer was dried with sodium sulfate, filtered, and concentrated. The crude material was purified by silica gel chromatography (Gradient: 0-50% EtOAc in heptane) to provide tert-butyl (2′S,4R)-2-formyl-2′-methyl-spiro[6,7-dihydrothieno[3,2-c]pyran-4,4′-piperidine]-1′-carboxylate C26 (29 mg, 65%). ¹H NMR (400 MHz, Chloroform-d) δ 9.82 (s, 1H), 7.42 (s, 1H), 4.05-3.85 (m, 3H), 3.76 (ddd, J=13.8, 6.1, 4.8 Hz, 1H), 3.37 (ddd, J=14.0, 8.9, 5.4 Hz, 1H), 2.96-2.80 (m, 2H), 2.10 (dddd, J=14.7, 8.2, 6.0, 1.8 Hz, 1H), 1.99 (ddd, J=14.2, 5.2, 1.9 Hz, 1H), 1.83-1.73 (m, 2H), 1.49 (s, 9H), 1.27 (d, J=6.6 Hz, 3H). LCMS m/z 351.93 [M+H]⁺.

### Step 2. Synthesis of tert-butyl (2′S,4R)-2-(difluoromethyl)-2′-methyl-spiro[6,7-dihydrothieno[3,2-c]pyran-4,4′-piperidine]-1′-carboxylate (C27)

A mixture of tert-butyl (2′S,4R)-2-formyl-2′-methyl-spiro[6,7-dihydrothieno[3,2-c]pyran-4,4′-piperidine]-1′-carboxylate C26 (29 mg, 0.08040 mmol) in DCM (500 μL) was stirred at rt. N-ethyl-N-(trifluoro-lambda4-sulfanyl)ethanamine (30 μL, 0.2271 mmol) and the mixture was stirred at reflux. After stirring 20 h, the mixture was cooled to rt, diluted with water, and the layers were separated. The organic layer was dried with sodium sulfate, filtered, concentrated, and purified by silica gel chromatography (Gradient: 0-50% EtOAc in heptane) to provide tert-butyl (2′S,4R)-2-(difluoromethyl)-2′-methyl-spiro[6,7-dihydrothieno[3,2-c]pyran-4,4′-piperidine]-1′-carboxylate C27 (18 mg, 59%). ¹H NMR (400 MHz, Chloroform-d) δ 6.90 (d, J=2.1 Hz, 1H), 6.75 (t, J=56.2 Hz, 1H), 4.03-3.83 (m, 3H), 3.74 (ddd, J=13.9, 6.0, 4.8 Hz, 1H), 3.33 (ddd, J=14.1, 8.9, 5.4 Hz, 1H), 2.88-2.72 (m, 2H), 2.05 (dddd, J=14.8, 9.0, 6.1, 1.8 Hz, 1H), 1.95 (ddd, J=14.2, 5.2, 1.8 Hz, 1H), 1.80-1.67 (m, 2H), 1.48 (s, 9H), 1.25 (d, J=6.5 Hz, 3H). LCMS m/z 373.99 [M+H]⁺.

### Step 3. Synthesis of (2′S,4R)-2-(difluoromethyl)-2′-methyl-1′-[[1-(2-methylsulfonylethyl)triazol-4-yl]methyl]spiro[6,7-dihydrothieno[3,2-c]pyran-4,4′-piperidine](524)

To a mixture of tert-butyl (2′S,4R)-2-(difluoromethyl)-2′-methyl-spiro[6,7-dihydrothieno[3,2-c]pyran-4,4′-piperidine]-1′-carboxylate C27 (18 mg, 0.04775 mmol) in DCM (400 μL) was added trifluoroacetic acid (40 μL, 0.5192 mmol) (11:30). After stirring 60 min, the mixture was dried, and redissolved in acetonitrile (1,000 μL) and to it was added 4-(chloromethyl)-1-(2-methylsulfonylethyl)triazole (Hydrochloride salt) (15 mg, 0.05766 mmol) and potassium carbonate (20 mg, 0.1447 mmol), and the mixture was stirred at 60° C. overnight. The mixture was diluted with EtOAc (5 mL) and water (2 mL), and the organic layer was mixed and separated. The organic layer was then washed with brine, dried with magnesium sulfate, filtered and concentrated. The crude material was purified by silica gel chromatography (Gradient: 0-10% MeOH in DCM) to provide (2′S,4R)-2-(difluoromethyl)-2′-methyl-1′-[[1-(2-methylsulfonylethyl)triazol-4-yl]methyl]spiro[6,7-dihydrothieno[3,2-c]pyran-4,4′-piperidine]524 (10 mg, 45%). ¹H NMR (400 MHz, Chloroform-d) δ 7.57 (s, 1H), 6.90 (t, J=2.1 Hz, 1H), 6.68 (t, J=56.1 Hz, 1H), 4.86-4.74 (m, 2H), 3.97 (d, J=14.7 Hz, 1H), 3.91-3.71 (m, 3H), 3.71-3.62 (m, 2H), 2.82-2.64 (m, 3H), 2.62 (d, J=0.7 Hz, 3H), 2.52 (dt, J=11.5, 5.9 Hz, 2H), 1.86-1.72 (m, 3H), 1.61 (t, J=12.6 Hz, 1H), 1.17 (d, J=6.2 Hz, 3H). LCMS m/z 461.1 [M+H]⁺.

### Compound 525

### (2′S,4R)-2′-methyl-1′-[[1-(2-methylsulfonylethyl)triazol-4-yl]methyl]-2-(trifluoromethyl)spiro[6,7-dihydrothieno[3,2-c]pyran-4,4′-piperidine]

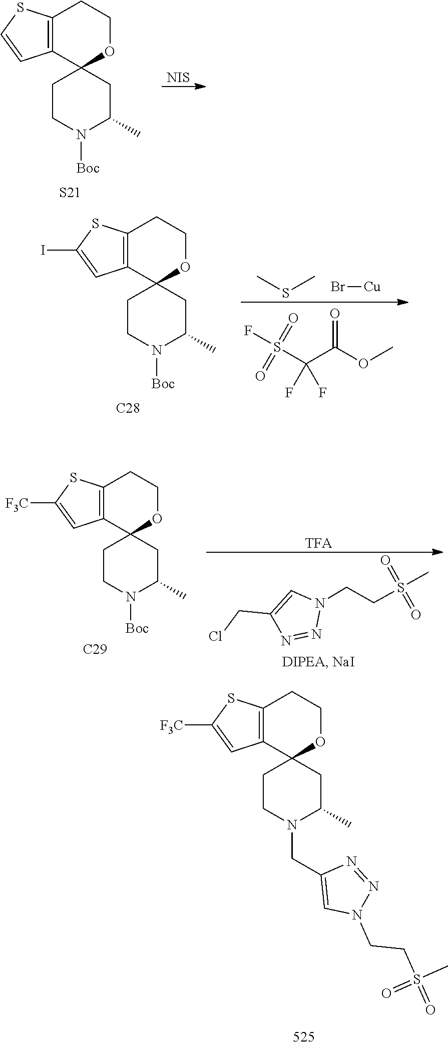

### Step 1. Synthesis of tert-butyl (2′S)-2-iodo-2′-methyl-spiro[6,7-dihydrothieno[3,2-c]pyran-4,4′-piperidine]-1′-carboxylate (C28)

Tert-butyl (2′S)-2′-methylspiro[6,7-dihydrothieno[3,2-c]pyran-4,4′-piperidine]-1′-carboxylate S21 (200 mg, 0.6183 mmol) was dissolved in CH₃CN (4 mL) and NIS (168 mg, 0.7467 mmol) added. The reaction mixture was stirred at rt overnight and then diluted with 1N NaOH/EtOAc and the organic layer was dried and concentrated, which was purified by silica gel chromatography (Gradient: 0 to 40% EtOAc/heptane) to provide tert-butyl (2′S)-2-iodo-2′-methyl-spiro[6,7-dihydrothieno[3,2-c]pyran-4,4′-piperidine]-1′-carboxylate C28 (222 mg, 80%). LCMS m/z 449.94 [M+H]⁺.

### Step 2. Synthesis of tert-butyl (2′S)-2′-methyl-2-(trifluoromethyl)spiro[6,7-dihydrothieno[3,2-c]pyran-4,4′-piperidine]-1′-carboxylate (C29)

Tert-butyl (2′S)-2-iodo-2′-methyl-spiro[6,7-dihydrothieno[3,2-c]pyran-4,4′-piperidine]-1′-carboxylate C28 (215 mg, 0.4785 mmol) was dissolved in DMF (4 mL) and bromocopper; methylsulfanylmethane (30 mg, 0.1459 mmol) and methyl 2,2-difluoro-2-fluorosulfonyl-acetate (150 μL, 1.178 mmol) were added. The reaction mixture was heated in the microwave to 100° C. for 40 min. Additional methyl 2,2-difluoro-2-fluorosulfonyl-acetate (75 μL, 0.5891 mmol) was added and the reaction mixture again was heated in the microwave to 100° C. for 40 min. The reaction mixture was diluted with EtOAc/1N NaOH and filtered through Celite®. The organic layer was dried and concentrated to an oil, which was purified by silica gel chromatography (Gradient: 0 to 25% EtOAc in heptane) to give tert-butyl (2′S)-2′-methyl-2-(trifluoromethyl)spiro[6,7-dihydrothieno[3,2-c]pyran-4,4′-piperidine]-1′-carboxylate C28 (87 mg, 46%). 1H NMR (300 MHz, Chloroform-d) δ 7.07 (s, 1H), 4.07-3.96 (m, 1H), 3.96-3.86 (m, 2H), 3.84-3.70 (m, 1H), 3.41-3.28 (m, 1H), 2.88-2.78 (m, 2H), 2.15-1.91 (m, 2H), 1.83-1.67 (m, 2H), 1.48 (s, 9H), 1.28 (d, J=6.5 Hz, 3H).

### Step 3. Synthesis of (2′S,4R)-2′-methyl-1′-[[1-(2-methylsulfonylethyl)triazol-4-yl]methyl]-2-(trifluoromethyl)spiro[6,7-dihydrothieno[3,2-c]pyran-4,4′-piperidine](525)

Tert-butyl (2′S)-2′-methyl-2-(trifluoromethyl)spiro[6,7-dihydrothieno[3,2-c]pyran-4,4′-piperidine]-1′-carboxylate C29 (42 mg, 0.1073 mmol) was dissolved in DCM (1 mL) and TFA (500 μL, 6.490 mmol) was added. After 20 min, the solvent was removed and the residue was redissolved in DCE (1 mL) and 4-(chloromethyl)-1-(2-methylsulfonylethyl)triazole (40 mg, 0.1788 mmol), NaI (3 mg, 0.02001 mmol) and DIPEA (60 μL, 0.3445 mmol) were added. The reaction mixture was heated to 60° C. for 20 h. The reaction mixture was concentrated, dissolved in 1 mL MeOH and repurified using reverse phase C18 column (Gradient: CH₃CN/H2O, TFA modifier). The pure fractions were diluted with EtOAc/1N NaOH and the organic layer was dried and concentrated to give (2′S,4R)-2′-methyl-1′-[[1-(2-methylsulfonylethyl)triazol-4-yl]methyl]-2-(trifluoromethyl)spiro[6,7-dihydrothieno[3,2-c]pyran-4,4′-piperidine]525 (27 mg, 52%) 1H NMR (300 MHz, Chloroform-d) δ 7.65 (s, 1H), 7.14 (s, 1H), 4.88 (t, J=6.3 Hz, 2H), 4.12-3.82 (m, 4H), 3.75 (t, J=6.3 Hz, 2H), 2.91-2.75 (m, 3H), 2.71 (s, 3H), 2.66-2.51 (m, 2H), 1.96-1.68 (m, 4H), 1.25 (d, J=6.2 Hz, 3H). LCMS m/z 479.11 [M+H]⁺.

### Compound 526 and 527

### (2′S,4R)-3-bromo-2-ethyl-2′-methyl-1′-[[1-(2-methylsulfonylethyl)triazol-4-yl]methyl]spiro[6,7-dihydrothieno[3,2-c]pyran-4,4′-piperidine](526) and (2′S,4R)-3-deuterio-2-ethyl-2′-methyl-1′-[[1-(2-methylsulfonylethyl)triazol-4-yl]methyl]spiro[6,7-dihydrothieno[3,2-c]pyran-4,4′-piperidine](527)

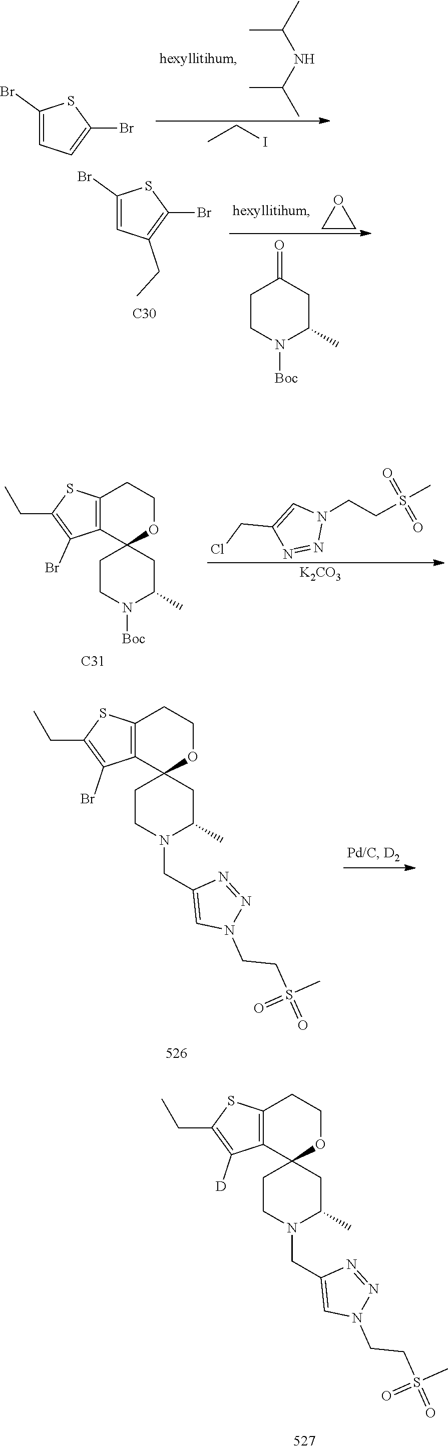

### Step 1. Synthesis of 3,5-dibromo-2-ethyl-thiophene (C30)

To a solution of N-isopropylpropan-2-amine (1.2 mL, 8.562 mmol) in THF (50 mL) at 0° C. under nitrogen was added hexyllithium (4.2 mL of 2.3 M, 9.660 mmol) over 5 min and the reaction was stirred for an additional 30 min. The solution was cooled to −78° C. before adding in 2,5-dibromothiophene (900 μL, 7.987 mmol). Then iodoethane (1.3 mL, 16.25 mmol) was added and stirred overnight with slowly warming to rt. The reaction was quenched with saturated ammonium chloride solution then extracted with DCM (3×100 mL) and washed the combined organics with saturated bicarbonate solution and saturated brine solution. The combined organic layer was evaporated in vacuo to yield 3,5-dibromo-2-ethyl-thiophene (1.1 g, 52%). ¹H NMR (300 MHz, Chloroform-d) δ 6.79 (s, 1H), 2.68 (q, J=7.5 Hz, 2H), 1.18 (t, J=7.5 Hz, 3H).

### Step 2. Synthesis of (2′S)-3-bromo-2-ethyl-2′-methyl-spiro[6,7-dihydrothieno[3,2-c]pyran-4,4′-piperidine](C31)

Dissolved 3,5-dibromo-2-ethyl-thiophene C30 (500 mg, 1.852 mmol) in THF (10 mL) at −78° C. Added hexyllithium (900 μL of 2.3 M, 2.070 mmol) and stirred for 30 min before adding oxirane (1000 μL of 2.5 M, 2.500 mmol). The reaction mixture was allowed to warm to rt and stir for 2 days. The reaction was quenched with saturated ammonium chloride solution and extracted with DCM (×3). The combined organic layer was concentrated in vacuo and purified by silica gel chromatography (Gradient: 0 to 60% EtOAc in heptane) to yield 2-(4-bromo-5-ethyl-2-thienyl)ethanol (200 mg, 46%). ¹H NMR (300 MHz, Chloroform-d) δ 6.62 (s, 1H), 3.78 (m, 2H), 2.91 (t, J=6.1 Hz, 2H), 2.73-2.61 (m, 2H), 1.18 (td, J=7.5, 1.4 Hz, 3H).

The material from step 2 was dissolved in dioxane (5 mL) and added tert-butyl (2S)-2-methyl-4-oxo-piperidine-1-carboxylate (400 mg, 1.876 mmol) under nitrogen. Added trifluoromethanesulfonic acid (400 μL, 4.520 mmol) and stirred overnight. The reaction mixture was quenched with saturated bicarbonate solution, diluted with DCM and extracted on a phase separator (×3). The solvent was evaporated under positive nitrogen pressure to yield (2′S)-3-bromo-2-ethyl-2′-methyl-spiro[6,7-dihydrothieno[3,2-c]pyran-4,4′-piperidine](Trifluoromethanesulfonic Acid (1)) C31 (510 mg, 37%). LCMS m/z 330.05 [M+H]⁺.

### Step 3. Synthesis of (2′S,4R)-3-bromo-2-ethyl-2′-methyl-1′-[[1-(2-methylsulfonylethyl)triazol-4-yl]methyl]spiro[6,7-dihydrothieno[3,2-c]pyran-4,4′-piperidine](526)

To a solution of (2′S)-3-bromo-2-ethyl-2′-methyl-spiro[6,7-dihydrothieno[3,2-c]pyran-4,4′-piperidine](Trifluoromethanesulfonic Acid (1)) C31 (120 mg, 0.1593 mmol) in DMF (1 mL) was added with K₂CO₃ (88 mg, 0.6367 mmol) and 4-(chloromethyl)-1-(2-methylsulfonylethyl)triazole (Hydrochloride salt) (42 mg, 0.1615 mmol). Reaction was stirred at 60° C. overnight. The reaction mixture was quenched with water and extracted with DCM (×3). The combined organic layer was concentrated in vacuo and then purified by silica gel chromatography (Gradient: 0-20% MeOH in DCM) to yield (2′S,4R)-3-bromo-2-ethyl-2′-methyl-1′-[[1-(2-methylsulfonylethyl)triazol-4-yl]methyl]spiro[6,7-dihydrothieno[3,2-c]pyran-4,4′-piperidine]526 (60 mg, 71%). LCMS m/z 517.1 [M+H]⁺.

### Step 4. Synthesis of (2′S,4R)-3-deuterio-2-ethyl-2′-methyl-1′-[[1-(2-methylsulfonylethyl)triazol-4-yl]methyl]spiro[6,7-dihydrothieno[3,2-c]pyran-4,4′-piperidine](527)

(2′S,4R)-3-bromo-2-ethyl-2′-methyl-1′-[[1-(2-methylsulfonylethyl)triazol-4-yl]methyl]spiro[6,7-dihydrothieno[3,2-c]pyran-4,4′-piperidine]526 (10 mg) in MeOH (2 mL) and added Pd/C (10 mg, 0.09397 mmol) under nitrogen. The reaction was subjected under deuterium gas overnight. Then the flask was purged with nitrogen and filtered off Pd/C to yield (2′S,4R)-3-deuterio-2-ethyl-2′-methyl-1′-[[1-(2-methylsulfonylethyl)triazol-4-yl]methyl]spiro[6,7-dihydrothieno[3,2-c]pyran-4,4′-piperidine]527 (6.0 mg, 73%). LCMS m/z 440.12 [M+H]⁺.

### Preparation S26

### 1-(5-chloro-2-thienyl)propan-2-ol (S26)

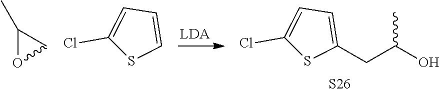

### Step 1: 1-(5-chloro-2-thienyl)propan-2-ol (S26)

To a stirred solution of 2-chlorothiophene (1.5 g, 12.6 mmol) in THF (20 mL) was added LDA (9.45 mL of 2 M solution in THF, 18.9 mmol) at −78° C. and the reaction mixture was stirred for 1 h. 2-methyloxirane (731 mg, 12.6 mmol) was added, and stirring was continued at −78° C. for 2 hours. The reaction was quenched with saturated aqueous NH₄Cl (50 mL) and extracted with EtOAc (2×50 mL). The combined organic layers were washed with brine (40 mL), dried over Na₂SO₄, filtered and concentrated. Purification by silica gel chromatography (Gradient: 5% EtOAc in heptane) gave 1-(5-chloro-2-thienyl)propan-2-ol S26 (1.3 g, 52%). ¹H NMR (300 MHz, DMSO-d6) δ 6.90 (d, J=3.6 Hz, 1H), 6.70 (d, J=3.9 Hz, 1H), 4.82 (d, J=4.8 Hz, 1H), 3.77 (m, 1H), 2.84-2.68 (m, 2H), 1.06 (d, J=5.7 Hz, 3H). LCMS m/z 272.19 [M+H]⁺.

### Intermediates S27-S29

Intermediates S27-S29 (Table 18) were prepared from 2-chlorothiophene and the appropriate epoxide as described in the method for compound S26.

TABLE 18
Structure and LCMS data of thiophene cores S27-S29

| Compound | Thiophene | Epoxide | ¹H NMR; LCMS m/z [M + H]⁺ |
|---|---|---|---|
| S27 | 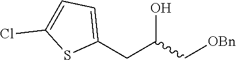 [SMILES: OC(COCc1ccccc1)Cc1ccc(Cl)s1] | 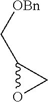 [SMILES: c1ccc(COCC2CO2)cc1] | ¹H NMR (300 MHz, DMSO-d6) δ 7.35-7.28 (m, 5H), 6.90 (d, J = 3.6 Hz, 1H), 6.71 (d, J = 4.2 Hz, 1H), 5.12 (d, J = 5.1 Hz, 1H), 4.52 (s, 1H), 4.48 (s, 1H), 3.80- 3.74 (m, 1H), 3.39-3.27 (m, 2H), 2.97 (dd, J = 14.7 HZ, 1H), 2.77- |
| | | | 2.69 (m, 1H). LCMS m/z 288.1 |
| S28 | 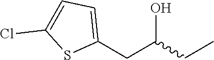 | 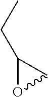 | ¹H NMR (400 MHz, CDCl₃) δ 6.895 (d, J = 4 Hz, 1H), 6.63- 6.62 (m, 1H), 3.72-3.69 (m, 1H), 2.96-2.91 (m, 1H), 2.81-2.75 (m, 1H), 1.66-1.51 (m, 2H), 0.98 (t, J = 4.8 Hz, 3H). LCMS m/z 288.24 [M + H]⁺. |
| S29 | 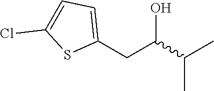 | 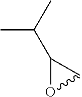 | ¹H NMR (300 MHz, DMSO-d6) δ 6.89 (d, J = 3.6 Hz, 1H), 6.725 (d, J = 3.6 Hz, 1H), 4.80 (d, J = 6 Hz, 1H), 3.34-3.30 (m, 1H), 2.87 (dd, J = 3.3, 15, 1H), 2.67 (dd, J = 8.4, 15, 1H), 1.59-1.45 (m, 1H), 0.86 (d, J = 6.6 Hz, |
| | | | 6H). LCMS m/z 300.15 [M + H]⁺. |

### Preparation S30

### (2′S)-2-chloro-2′,6-dimethyl-spiro[6,7-dihydrothieno[3,2-c]pyran-4,4′-piperidine](S30)

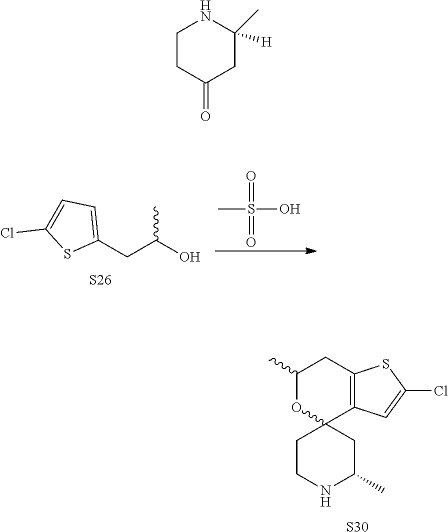

### Step 1: Synthesis of (2′S)-2-chloro-2′,6-dimethyl-spiro[6,7-dihydrothieno[3,2-c]pyran-4,4′-piperidine](S30)

To a stirred solution of 1-(5-chloro-2-thienyl)propan-2-ol S30 (1.3 g, 0.0066 mol), (2S)-2-methylpiperidin-4-one (Trifluoroacetic Acid (1)) (1.5 g, 0.0059 mol) in Toluene (15 mL) was added Methane sulfonic acid (701.58 mg, 0.4737 mL, 0.0073 mol) at room temperature. The reaction mixture was stirred at 120° C. for 4 h. The pH was adjusted to 8-9 using a saturated aqueous solution of Na₂CO₃ and extracted with ethyl acetate (2×50 mL). The combined organic layer was washed with brine (50 mL) dried over anhydrous Na₂SO₄, filtered, and concentrated. Purification by reversed-phase HPLC. Method Xbridge C18 column (19×150 mm, 5 micron). Gradient: MeCN in H2O with 10 mM Ammonium bicarbonate yielded the product. The collected fractions were lyophilize to afford (2′S)-2-chloro-2′,6-dimethyl-spiro[6,7-dihydrothieno[3,2-c]pyran-4,4′-piperidine]S30 (370 mg, 20%) ¹H NMR (300 MHz, DMSO-d6) δ 6.92 (s, 1H), 3.87 (d, J=9.6 Hz, 1H), 2.95-2.78 (m, 2H), 2.71-2.62 (m, 2H), 2.42-2.33 (m, 1H), 1.91-1.79 (m, 2H), 1.46-1.36 (m, 2H), 1.23 (d, J=6.3 Hz, 3H), 1.15-1.07 (m, 1H), 0.94-0.91 (m, 3H). LCMS m/z 272.11 [M+1]⁺.

### Compounds S31-S33

Compounds S31-S33 (Table 19) were prepared from (2S)-2-methylpiperidin-4-one and the appropriate thiophene reagent as described in the method for compound S30.

TABLE 19
Structure and LCMS data of spiropiperidine cores S31-S33

| Compd | Structure | Thiophene | LCMS m/z [M + H]⁺ |
|---|---|---|---|
| S31¹ | 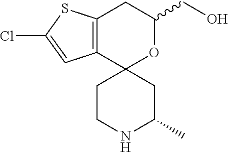 [SMILES: C[C@H]1CC2(CCN1)OC(CO)Cc1sc(Cl)cc12] | 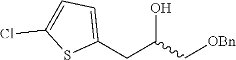 [SMILES: OC(COCc1ccccc1)Cc1ccc(Cl)s1] | LCMS m/z 288.1 [M + H]⁺. |
| S32 | 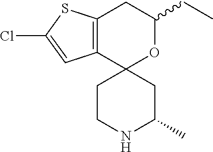 [SMILES: CCC1Cc2sc(Cl)cc2C2(CCN[C@@H](C)C2)O1] | 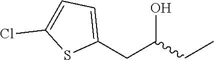 | LCMS m/z 288.24 [M + H]⁺. |
| S33 | 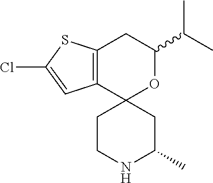 [SMILES: CC(C)C1Cc2sc(Cl)cc2C2(CCN[C@@H](C)C2)O1] | 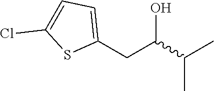 | LCMS m/z 300.15 [M + H]⁺. |

Footnotes:
¹)Trifluoro methane sulfonic acid (1 eq.) was used as an acid.

### Compound 528

### (2′S)-2-chloro-2′,6-dimethyl-1′-[(1-methylpyrazol-4-yl)methyl]spiro[6,7-dihydrothieno[3,2-c]pyran-4,4′-piperidine](528 [DIAST-1] and 529 [DIAST-2])

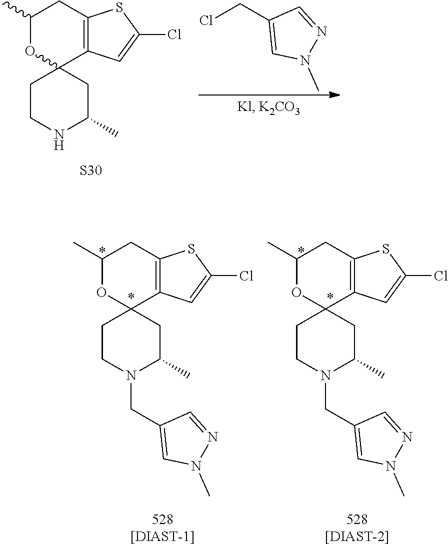

### Preparation of (2′S)-2-chloro-2′,6-dimethyl-1′-[(1-methylpyrazol-4-yl)methyl]spiro[6,7-dihydrothieno[3,2-c]pyran-4,4′-piperidine](528 and 529)

To a stirred solution of (2′S)-2-chloro-2′,6-dimethyl-spiro[6,7-dihydrothieno[3,2-c]pyran-4,4′-piperidine]S30 (300 mg, 0.993 mmol) and 4-(chloromethyl)-1-methyl-pyrazole (115 mg, 0.792 mmol) in MeCN (6 mL) was added potassium carbonate (691 mg, 5.0 mmol) followed by KI (33 mg, 0.198 mmol) and stirred at room temperature for 16 h. Upon completion, the solvent was evaporated. Purification by reversed-phase HPLC. Method: XSelect Phenyl hexyl column (19×250 mm, 5 micron). Gradient: MeCN in H2O with 0.1% formic acid. followed by SFC purification gave two diastereomers:

(2′S)-2-chloro-2′,6-dimethyl-1′-[(1-methylpyrazol-4-yl)methyl]spiro[6,7-dihydrothieno[3,2-c]pyran-4,4′-piperidine]528 [DIAST-1](39.7 mg, 11%) ¹H NMR (400 MHz, DMSO-d6) δ 7.54 (s, 1H), 7.28 (s, 1H), 6.98 (s, 1H), 3.82-3.79 (m, 4H), 3.73 (d, J=14 Hz, 1H), 3.25 (d, J=13.6 Hz, 1H), 2.63 (dd, J=3.2 Hz and 16 Hz, 1H), 2.55-2.45 (m, 2H), 2.39-2.28 (m, 2H), 1.87 (dd, J=2.4 Hz and 14 Hz, 1H), 1.75-1.69 (m, 1H), 1.57-1.56 (m, 1H), 1.46-1.42 (m, 1H), 1.18 (d, J=6 Hz, 3H), 1.07 (d, J=6 Hz, 3H). LCMS m/z 366.1 [M+1]⁺.; and

(2′S)-2-chloro-2′,6-dimethyl-1′-[(1-methylpyrazol-4-yl)methyl]spiro[6,7-dihydrothieno[3,2-c]pyran-4,4′-piperidine]529 [DIAST-2](10.3 mg, 3%) ¹H NMR (400 MHz, DMSO-d6) δ 7.54 (s, 1H), 7.28 (s, 1H), 6.98 (s, 1H), 3.90-3.85 (m, 1H), 3.79-3.72 (m, 4H), 3.32-3.23 (m, 1H), 2.64 (dd, J=2.8 Hz and 16 Hz, 1H), 2.56-2.49 (m, 1H), 2.41-2.32 (m, 3H), 1.97-1.90 (m, 2H), 1.44-1.34 (m, 2H), 1.18 (d, J=6 Hz, 3H), 1.09 (d, J=6 Hz, 3H). LCMS m/z 366.1 [M+1]⁺.

### Compounds 530-533

Compounds 530-533 (Table 20) were prepared from the appropriately chosen piperidine and 4-(chloromethyl)-1-methyl-pyrazole using the method for compound 528 and 529.

TABLE 20
Structure and physicochemical data for compounds 530-533

| Compd | Structure | Piperidine | ¹H NMR; LCMS m/z [M + H]⁺ |
|---|---|---|---|
| 530 | 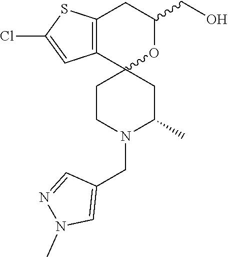 | S31¹ | ¹H NMR (400 MHz, DMSO-d6) δ 7.54 (s, 1H), 7.27 (s, 1H), 6.98 (s, 1H), 4.7 (br s, 1H), 3.79-3.73 (m, 5H), 3.56-3.48 (m, 1H), 3.43-3.32 (m, 1H), 3.23 (d, J = 14 Hz, 1H), 2.66-2.60 (m, 2H), 2.46-2.32 (m, 3H), 1.73-1.41 (m, 4H), 1.09-1.06 (m, 3H). LCMS m/z 382.2 [M + H]⁺. |
| 531 | 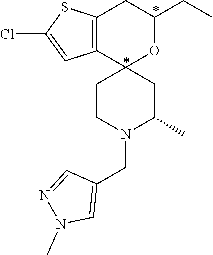 | S32 | ¹H NMR (400 MHz, DMSO-d6) δ 7.59 (brs, 1H), 7.31 (brs, 1H), 6.95 (s, 1H), 3.79-3.76 (m, 4H), 3.68-3.58 (m, 1H), 3.42-3.32 (m, 1H), 2.64 (d, J = 16, 3H), 2.38-2.31 (m, 2H), 1.99 (brs, 2H), 1.52-1.44 (m, 4H), 1.19-1.15 (m, 3H), 0.83 (t, J = 4.8 Hz, 3H). LCMS m/z 380.0 [M + H]⁺. |
| 532 | 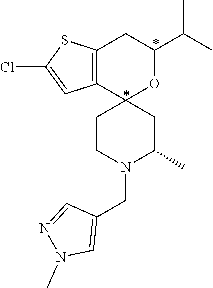 | S33 | ¹H NMR (400 MHz, DMSO-d6) δ 7.53 (s, 1H), 7.27 (s, 1H), 6.97 (s, 1H), 3.77 (s, 3H), 3.69 (d, J = 14 Hz, 1H), 3.38-3.31- (m, 2H), 2.70-2.65 (m, 1H), 2.51-2.49 (m, 1H), 2.46-2.34 (m, 3H), 1.98-1.89 (m, 2H), 1.62-1.61 (m, 1H), 1.43-1.32 (m, 2H), 1.08 (d, J = 6.4 Hz, 3H), 0.87-0.85 (m, 6H). LCMS m/z 394.2 [M + H]⁺. |
| 533 | 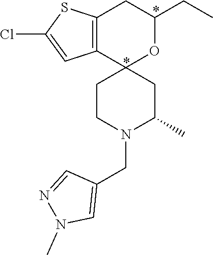 | S32 | ¹H NMR (400 MHz, DMSO-d6) δ 7.53 (s, 1H), 7.27 (s, 1H), 6.97 (s, 1H), 3.78 (s, 3H), 2.64 (d, J = 14, 1H), 3.61-3.52 (m, 1H), 3.38-3.31 (m, 1H), 2.64-2.60 (m, 1H), 2.51-2.49 (m, 2H), 2.38-2.31 (m, 2H), 1.88 (br d, 1H), 1.756 (t, J = 13.2 Hz, 1H), 1.61-1.42 (m, 4H), 1.07 (d, J = 6, 3H), 0.86 (t, J = 7.8 Hz, 3H). LCMS m/z 380.0 [M + H]⁺. |

### Preparation S34

### (2′S)-2′,7-dimethyl-2-(trifluoromethyl)spiro[6,7-dihydrothieno[3,2-c]pyran-4,4′-piperidine](S34)

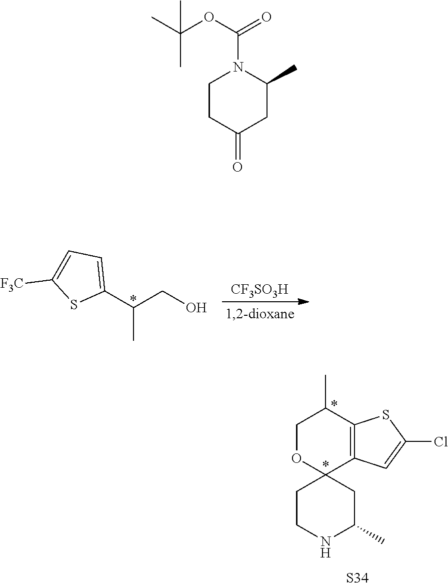

### Step 1: Synthesis of (2′S)-2′,7-dimethyl-2-(trifluoromethyl)spiro[6,7-dihydrothieno[3,2-c]pyran-4,4′-piperidine](S34)

2-[5-(trifluoromnethyl)-2-thienyl]propan-1-ol (250 mg, 1.18 mmol) and tert-butyl (2S)-2-methyl-4-oxo-piperidine-1-carboxylate (362 mg, 1.69 mmol) were dissolved in 1,2-dioxane (3 mL). Trifluoromethanesulfonic acid (375 μL, 4.24 mmol) was added and stirred at rt overnight. The reaction was quenched with NaHCO₃ and solvent was evaporated. DCM and water were added and the organic layer was collected through phase separator. The solvent was evaporated to give (2′S)-2′,7-dimethyl-2-(trifluoromnethyl)spiro[6,7-dihydrothieno[3,2-c]pyran-4,4′-piperidine](S34) (600 mg, 146%). LCMS m/z 306.24 [M+H]⁺.

### Compounds S35-S41

Compounds S35-S41 (Table 21) were prepared from tert-butyl (2S)-2-methyl-4-oxo-piperidine-1-carboxylate and the appropriate thiophene using the method for compound S34.

TABLE 22
Structure and physicochemical data for compounds S35-S41

| Cmpd | Structure | Thiophene | LCMS m/z [M + H]⁺ |
|---|---|---|---|
| S35¹ | 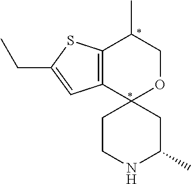 [SMILES: CCc1cc2c(s1)C(C)COC21CCN[C@@H](C)C1] | 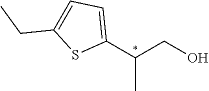 | LCMS m/z 266.03 [M + H]⁺. |
| S36 | 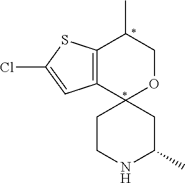 [SMILES: CC1COC2(CCN[C@@H](C)C2)c2cc(Cl)sc21] | 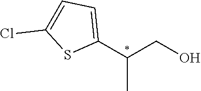 | LCMS m/z 272.2 [M + H]⁺. |
| S37¹ | 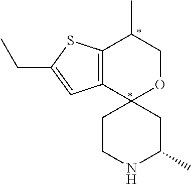 [SMILES: CCc1cc2c(s1)C(C)COC21CCN[C@@H](C)C1] | 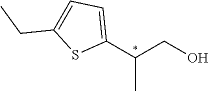 | LCMS m/z 266.03 [M + H]⁺. |
| S38¹ | 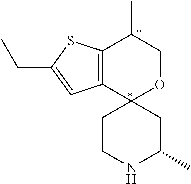 [SMILES: CCc1cc2c(s1)C(C)COC21CCN[C@@H](C)C1] | 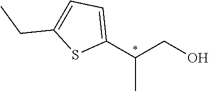 | LCMS m/z 266.21 [M + H]⁺. |
| S39¹ | 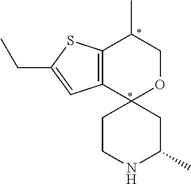 [SMILES: CCc1cc2c(s1)C(C)COC21CCN[C@@H](C)C1] | 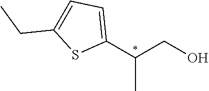 | LCMS m/z 266.21 [M + H]⁺. |
| S40 | 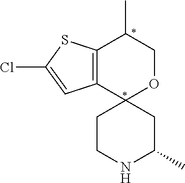 [SMILES: CC1COC2(CCN[C@@H](C)C2)c2cc(Cl)sc21] | 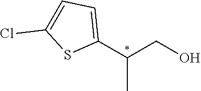 | LCMS m/z 272.2 [M + H]⁺. |
| S41 | 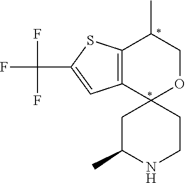 [SMILES: CC1COC2(CCN[C@@H](C)C2)c2cc(C(F)(F)F)sc21] | 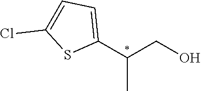 | LCMS m/z 306.24 [M + H]⁺. |
| S42 | 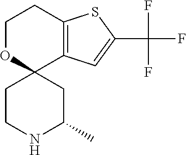 [SMILES: C[C@H]1C[C@@]2(CCN1)OCCc1sc(C(F)(F)F)cc12] | 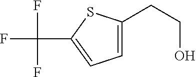 [SMILES: OCCc1ccc(C(F)(F)F)s1] | LCMS m/z 292.38 [M + H]⁺. |

Footnotes:
¹)(2′S)-2-ethyl-2′,7-dimethyl-spiro[6,7-dihydrothieno[3,2-c]pyran-4,4′-piperidine]was separated into constituent diastereomers (cis and trans) by chiral SFC separation. Column: Phenomenex Lux ® Cellulose-2, 20 × 250 mm. Mobile phase: 40% MeOH (containing 5 mM Ammonia), 60% CO2

### Compound 534

### (2S,4R)-2,7′-dimethyl-1-((1-(2-(methylsulfonyl)ethyl)-1H-1,2,3-triazol-4-yl)methyl)-2′-(trifluoromethyl)-6′,7′-dihydrospiro[piperidine-4,4′-thieno[3,2-c]pyran]

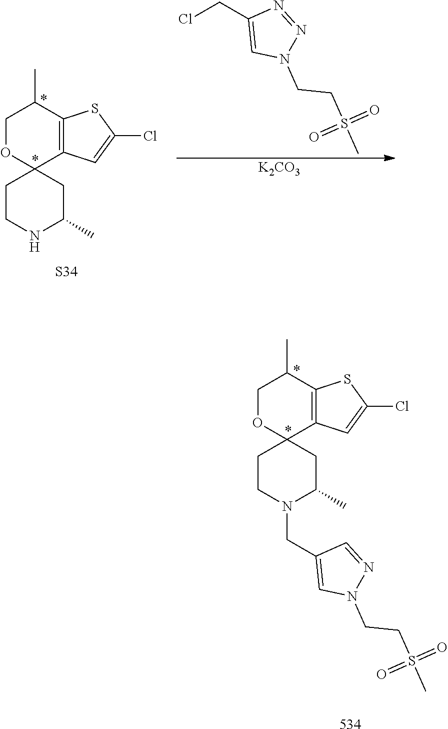 [SMILES: CC1COC2(CCN[C@@H](C)C2)c2cc(Cl)sc21 ; CS(=O)(=O)CCn1cc(CCl)nn1 ; "K2CO3" ; "S34" ; CC1COC2(CCN(Cc3cnn(CCS(C)(=O)=O)c3)[C@@H](C)C2)c2cc(Cl)sc21 ; "534"]

### Step 1: (2S,4R)-2,7′-dimethyl-1-((1-(2-(methylsulfonyl)ethyl)-1H-1,2,3-triazol-4-yl)methyl)-2′-(trifluoromethyl)-6′,7′-dihydrospiro[piperidine-4,4′-thieno[3,2-c]pyran] (534)

To a stirred solution of (2S,4R)-2,7′-dimethyl-2′-(trifluoromethyl)-6′,7′-dihydrospiro[piperidine-4,4′-thieno[3,2-c]pyran]S34 (71 mg, 0.111 mmol) and 4-(chloromethyl)-1-(2-methylsulfonylethyl)triazole (25 mg, 0.111 mmol) in DMF was added potassium carbonate (15 mg, 0.111 mmol) and reaction was stirred at 60° C. overnight. Water and dichloromethane were added and the organic layer was collected through phase separator. Purification by silica gel chromatography (Gradient: 0-20% Methanol in DCM) gave (2S,4R)-2,7′-dimethyl-1-((1-(2-(methylsulfonyl)ethyl)-1H-1,2,3-triazol-4-yl)methyl)-2′-(trifluoromethyl)-6′,7′-dihydrospiro[piperidine-4,4′-thieno[3,2-c]pyran]534 (10 mg, 18%). LCMS m/z 493.56 [M+H]⁺.

### Compounds 535-541

Compounds 535-541 (Table 23) were prepared from intermediate piperidines and 4-(chloromethyl)-1-(2-(methylsulfonyl)ethyl)-1H-1,2,3-triazole using the method for compound 534

TABLE 23
Structure and physicochemical data for compounds 535-541

| | | | ¹H NMR; |
|---|---|---|---|
| Cmpd | Structure | Piperidine | LCMS m/z [M + H]⁺ |
| 535 | 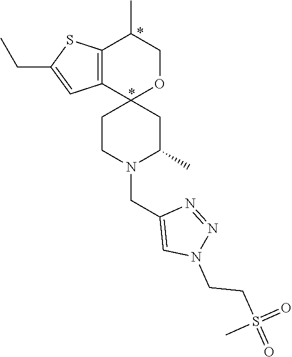 [SMILES: CCc1cc2c(s1)C(C)COC21CCN(Cc2cn(CCS(C)(=O)=O)nn2)[C@@H](C)C1] | 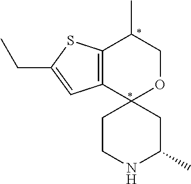 | ¹H NMR (400 MHz, DMSO-d6) δ 8.08 (s, 1H), 6.57 (s, 1H), 4.80 (m, 2H), 3.91-3.66 (m, 5H), 3.37 (m, 1H), 2.92 (s, 3H), 2.77 (m, 3H), 2.43 (m, 2H), 1.87-1.69 (m, 3H), 1.63-1.42 (m, 2H), 1.20-1.08 (m, 9H). LCMS m/z 453.2 [M + H]⁺. |
| 536 | 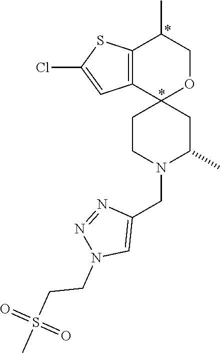 [SMILES: CC1COC2(CCN(Cc3cn(CCS(C)(=O)=O)nn3)[C@@H](C)C2)c2cc(Cl)sc21] | 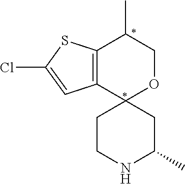 | LCMS m/z 459.17 [M + H]⁺. |
| 537 | 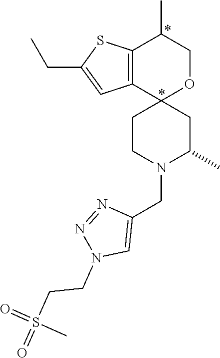 [SMILES: CCc1cc2c(s1)C(C)COC21CCN(Cc2cn(CCS(C)(=O)=O)nn2)[C@@H](C)C1] | 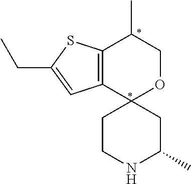 | ¹H NMR (400 MHz, DMSO-d6) δ 8.08 (s, 1H), 6.57 (s, 1H), 4.80 (t, J = 6.9 Hz, 2H), 3.94- 3.66 (m, 5H), 3.50-3.21 (m, 3H), 2.92 (s, 3H), 2.71 (m, 3H), 2.41 (m, 1H), 1.79-1.55 (m, 4H), 1.23-1.05 (m, 9H). LCMS m/z 453.45 [M + H]⁺. |
| 538 | 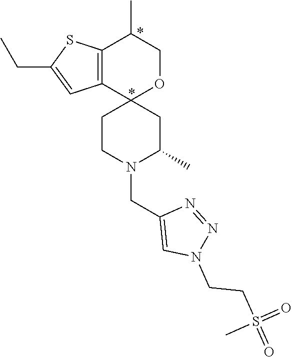 | 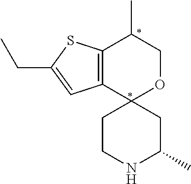 | ¹H NMR (400 MHz, DMSO-d6) δ 8.08 (s, 1H), 6.55 (s, 1H), 4.79 (t, J = 7.0 Hz, 2H), 3.91- 3.77 (m, 3H), 3.76-3.62 (m, 2H), 3.43 (dd, J = 11.4, 6.0 Hz, 1H), 2.98 (d, J = 14.3 Hz, 5H), 2.91-2.64 (m, 4H), 1.96- 1.61 (m, 4H), 1.22- 1.08 (m, 9H). LCMS m/z 493.17 [M + H]⁺. |
| 539 | 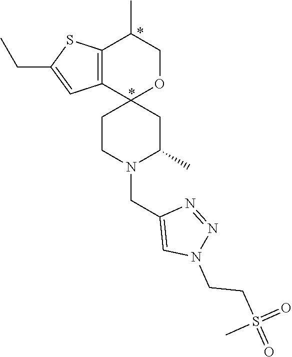 [SMILES: CCc1cc2c(s1)C(C)COC21CCN(Cc2cn(CCS(C)(=O)=O)nn2)[C@@H](C)C1] | 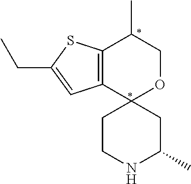 | LCMS m/z 453.45 [M + H]⁺. |
| 540 | 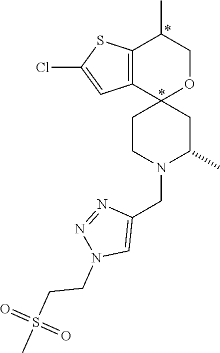 [SMILES: CC1COC2(CCN(Cc3cn(CCS(C)(=O)=O)nn3)[C@@H](C)C2)c2cc(Cl)sc21] | 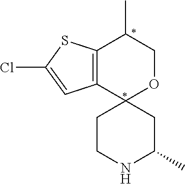 | LCMS m/z 459.12 [M + H]⁺. |
| 541 | 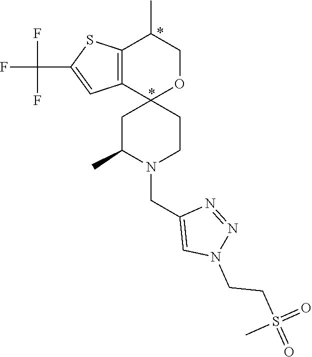 [SMILES: CC1COC2(CCN(Cc3cn(CCS(C)(=O)=O)nn3)[C@@H](C)C2)c2cc(C(F)(F)F)sc21] | 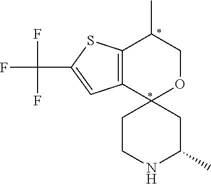 | LCMS m/z 493.15 [M + H]⁺ |

### Compound 542

### (2′S,4R)-2′-methyl-1′-[(1-methylpyrazol-4-yl)methyl]-2-(trifluoromethyl)spiro[6,7-dihydrothieno[3,2-c]pyran-4,4′-piperidine]-7-ol

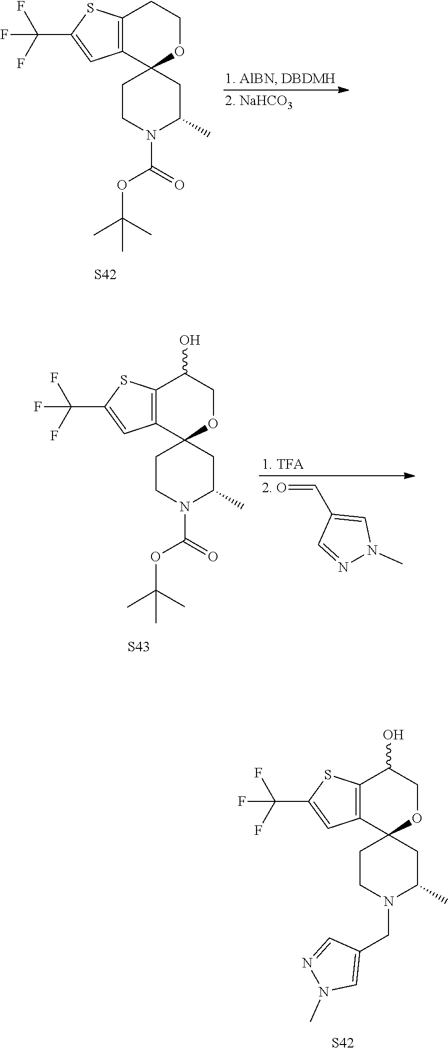

### Step 1: Synthesis of tert-butyl (2′S,4R)-7-hydroxy-2′-methyl-2-(trifluoromethyl)spiro[6,7-dihydrothieno[3,2-c]pyran-4,4′-piperidine]-1′-carboxylate (S43)

Tert-butyl (2′S,4R)-2′-methyl-2-(trifluoromethyl)spiro[6,7-dihydrothieno[3,2-c]pyran-4,4′-piperidine]-1′-carboxylate S42 (670 mg, 1.71 mmol), 1,3-dibromo-5,5-dimethyl-imidazolidine-2,4-dione (390 mg, 1.36 mmol) and 2-(1-cyano-1-methyl-ethyl)azo-2-methyl-propanenitrile (20 mg, 0.121 mmol) were combined in dichloromethane (10 mL) and the mixture was stirred at 35° C. for 4 h. Sodium thiosulfate was added and the reaction mixture was diluted with water and ethyl acetate. The organic layer was concentrated and purification by silica gel chromatography (Gradient: 0-30% EtOAc in heptane) gave bromide intermediate tert-butyl (2′S,4R)-7-bromo-2′-methyl-2-(trifluoromethyl)spiro[6,7-dihydrothieno[3,2-c]pyran-4,4′-piperidine]-1′-carboxylate. Intermediate was dissolved in THF and treated with saturated NaHCO₃ (2 mL) and the mixture was heated to 60° C. overnight. The mixture was diluted with water and EtOAc and the organic layer dried and concentrated. Purification by silica gel chromatography (Gradient: 0-50% EtOAc in heptane) tert-butyl (2′S,4R)-7-hydroxy-2′-methyl-2-(trifluoromethyl)spiro[6,7-dihydrothieno[3,2-c]pyran-4,4′-piperidine]-1′-carboxylate S43 (48 mg, 7%) ¹H NMR (300 MHz, Chloroform-d) δ 7.09 (s, 1H), 4.74-4.61 (m, 1H), 4.05-3.69 (m, 4H), 3.44-3.24 (m, 1H), 2.39 (t, J=9.0 Hz, 1H), 2.26-1.73 (m, 3H), 1.50 (s, 9H), 1.28 (d, J=6.6 Hz, 3H).

### Step 2: (2′S,4R)-2′-methyl-1′-[(1-methylpyrazol-4-yl)methyl]-2-(trifluoromethyl)spiro[6,7-dihydrothieno[3,2-c]pyran-4,4′-piperidine]-7-ol (542)

Tert-butyl (2′S,4R)-7-hydroxy-2′-methyl-2-(trifluoromethyl)spiro[6,7-dihydrothieno[3,2-c]pyran-4,4′-piperidine]-1′-carboxylate (48 mg, 0.117 mmol) was dissolved in dichloromethane (1 mL) and TFA (0.5 mL, 6.490 mmol) was added. The reaction mixture was stirred for 30 min then concentrated to an oil. The oil was dissolved in dichloromethane (1 mL) and 1-methylpyrazole-4-carbaldehyde (20 mg, 0.181 mmol), AcOH (35 μL, 0.615 mmol), and (trimethylammonio)methyl (cyanoborohydride) (180 mg of 2 mmol/g, 0.3600 mmol) resin were added. The reaction mixture was heated to 110° C. in microwave for 60 mins, The reaction was filtered and the filtrate concentrated. Purification by silica chromatography (Gradient: 0-20% Methanol in DCM) gave (2′S,4R)-2′-methyl-1′-[(1-methylpyrazol-4-yl)methyl]-2-(trifluoromethyl)spiro[6,7-dihydrothieno[3,2-c]pyran-4,4′-piperidine]-7-ol 542 (23 mg, 48%) ¹H NMR (300 MHz, Chloroform-d) δ 7.40 (s, 1H), 7.30 (s, 1H), 7.15 (s, 1H), 4.76-4.58 (m, 1H), 4.06-3.76 (m, 6H), 3.62-3.48 (m, 1H), 3.05-2.36 (m, 4H), 2.09-1.69 (m, 3H), 1.67-1.43 (m, 1H), 1.23-1.14 (m, 3H). LCMS m/z 402.04 [M+H]⁺.

### Preparation of S44

### (2′S, 7R)-2-(2,2-difluoroethyl)-2′-methyl-spiro[4,5-dihydrothieno[2,3-c]pyran-7,4′-piperidine](S44)

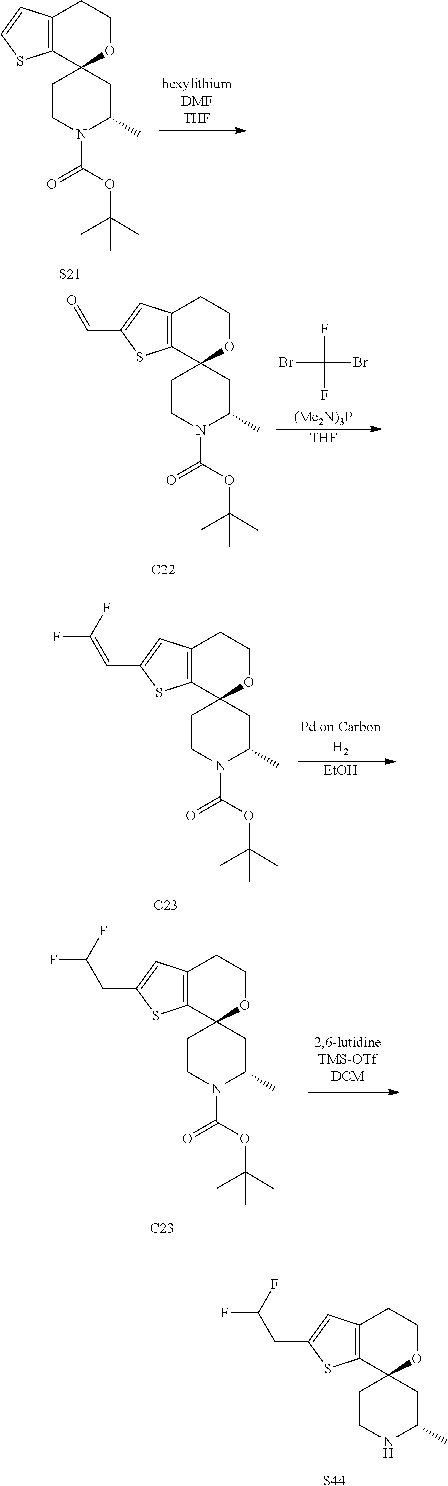

### Step 1. Synthesis of tert-butyl (2′S, 7R)-2-formyl-2′-methyl-spiro[4,5-dihydrothieno[2,3-c]pyran-7,4′-piperidine]-1′-carboxylate (C22)

To a solution of tert-butyl (2′S,7R)-2′-methylspiro[4,5-dihydrothieno[2,3-c]pyran-7,4′-piperidine]-1′-carboxylate S21 (81.4 g, 251.7 mmol) in THF (740 mL) cooled to −78° C. under nitrogen was added a solution of hexyllithium in hexane (120 mL of 2.6 M, 312.0 mmol) via an addition funnel over the course of 20 min. After stirring for 70 min following completion of addition of the hexyllithium, DMF (100 mL, 1.291 mol) was added over the course of 5 min. The solution was stirred at −78° C. for 30 min, then the reaction warmed to 0° C. and stirred for 45 min. The reaction was then quenched via addition of saturated aqueous ammonium chloride (600 mL). Mixture was partitioned between EtOAc (1 L) and water (500 mL). Organic layer was separated, washed with a saturated aqueous ammonium chloride solution, water, and brine (600 mL each). The organic layer was dried over magnesium sulfate, filtered, and concentrated to yield tert-butyl (2′S,7R)-2-formyl-2′-methyl-spiro[4,5-dihydrothieno[2,3-c]pyran-7,4′-piperidine]-1′-carboxylate C22 (88 g, 99%) as a viscous amber oil which was used without further purification. ¹H NMR (300 MHz, Chloroform-d) δ 9.83 (s, 1H), 7.42 (s, 1H), 4.01 (ddd, J=11.5, 6.7, 5.3 Hz, 1H), 3.94-3.82 (m, 2H), 3.75 (ddd, J=14.0, 6.1, 4.7 Hz, 1H), 3.35 (ddd, J=14.1, 8.8, 5.4 Hz, 1H), 2.71 (td, J=5.5, 2.8 Hz, 2H), 2.28-2.06 (m, 2H), 1.90-1.65 (m, 2H), 1.47 (s, 9H), 1.26 (d, J=6.6 Hz, 3H).

### Step 2. Synthesis of tert-butyl (2′S, 7R)-2-(2,2-difluorovinyl)-2′-methyl-spiro[4,5-dihydrothieno[2,3-c]pyran-7,4′-piperidine]-1′-carboxylate (C23)

To a solution of dibromo(difluoro)methane (33 mL, 361.3 mmol) in THF (600 mL) in a dry ice-acetone cooling bath was added N-[bis(dimethylamino)phosphanyl]-N-methyl-methanamine (140 mL, 770.3 mmol) over the course of 45 min via addition funnel. The dry-ice acetone bath was then replaced with an ice-water bath and stirred for 45 min. To the reaction was added a solution of tert-butyl (2′S,7R)-2-formyl-2′-methyl-spiro[4,5-dihydrothieno[2,3-c]pyran-7,4′-piperidine]-1′-carboxylate C22 (88 g, 250.4 mmol) in THF (500 mL) over the course of 30 min at which point the ice-water bath was removed. After 2 hours, the reaction was cooled in an ice-water bath then quenched via addition of water (300 mL) added over 5 min. An aqueous solution of 10% sodium bisulfite (300 mL) was added. The mixture was stirred for 30 min, then the organic layer was isolated. The organic layer was washed with 1 M aqueous HCl (1 L). The organic layer was diluted with MTBE (500 mL), washed with saturated aqueous sodium bicarbonate (1 L) followed by 50% saturated aqueous brine (1 L), dried over magnesium sulfate, filtered, and concentrated. The crude residue was purified by silica gel chromatography (Gradient: 0-50% EtOAc in heptanes to afford tert-butyl (2′S,7R)-2-(2,2-difluorovinyl)-2′-methyl-spiro[4,5-dihydrothieno[2,3-c]pyran-7,4′-piperidine]-1′-carboxylate C23 (54 g, 56%) as a viscous clear pale yellow oil. ¹H NMR (300 MHz, Chloroform-d) δ 6.62 (s, 1H), 5.44 (dd, J=25.8, 2.0 Hz, 1H), 4.00 (dp, J=12.2, 6.4 Hz, 1H), 3.86 (t, J=5.5 Hz, 2H), 3.74 (ddd, J=13.9, 6.0, 4.7 Hz, 1H), 3.32 (ddd, J=14.1, 8.9, 5.4 Hz, 1H), 2.71-2.51 (m, 2H), 2.26-2.03 (m, 2H), 1.91-1.65 (m, 2H), 1.48 (s, 9H), 1.25 (d, J=6.5 Hz, 3H). 19F NMR (282 MHz, Chloroform-d) 6-80.93 (d, J=28.1 Hz), −87.47 (d, J=27.9 Hz).

### Step 3. Synthesis of tert-butyl (2′S, 7R)-2-(2,2-difluoroethyl)-2′-methyl-spiro[4,5-dihydrothieno[2,3-c]pyran-7,4′-piperidine]-1′-carboxylate (C24)

To Pd on carbon (8.2 g of 5% w/w, 3.853 mmol) in a 2 L Parr bottle under nitrogen was added a solution of tert-butyl (2′S,7R)-2-(2,2-difluorovinyl)-2′-methyl-spiro[4,5-dihydrothieno[2,3-c]pyran-7,4′-piperidine]-1′-carboxylate C23 (54 g, 140.1 mmol) in EtOH (700 mL). The Parr shaker was charged with 50 psi of H₂. After 24 hours, the reaction mixture was filtered through a Celite pad and the filtrate was treated with fresh catalyst Pd on carbon (10 g of 5% w/w, 4.698 mmol) under nitrogen. The Parr shaker was charged with 50 psi H₂ and the reaction continued for an additional 16 hours. The reaction was then filtered through a Celite pad washing with EtOH (200 mL). The filtrate was concentrated to a yellow oil which was purified by silica gel chromatography (Gradient: 0-50% EtOAc in heptanes), to afford tert-butyl (2′S,7R)-2-(2,2-difluoroethyl)-2′-methyl-spiro[4,5-dihydrothieno[2,3-c]pyran-7,4′-piperidine]-1′-carboxylate (45.5 g, 84%) as a clear colorless viscous oil. ¹H NMR (300 MHz, Chloroform-d) δ 6.58 (s, 1H), 5.90 (tt, J=56.5, 4.4 Hz, 1H), 3.99 (ddd, J=11.5, 6.7, 5.3 Hz, 1H), 3.86 (t, J=5.5 Hz, 2H), 3.79-3.66 (m, 1H), 3.40-3.16 (m, 3H), 2.72-2.51 (m, 2H), 2.24-2.03 (m, 2H), 1.90-1.65 (m, 2H), 1.48 (s, 9H), 1.25 (d, J=6.5 Hz, 3H). 19F NMR (282 MHz, Chloroform-d) δ−115.12.

### Step 4. Synthesis of (2′S, 7R)-2-(2,2-difluoroethyl)-2′-methyl-spiro[4,5-dihydrothieno[2,3-c]pyran-7,4′-piperidine](S44)

To a solution of tert-butyl (2′S,7R)-2-(2,2-difluoroethyl)-2′-methyl-spiro[4,5-dihydrothieno[2,3-c]pyran-7,4′-piperidine]-1′-carboxylate (32 g, 82.58 mmol) C23 in dichloromethane (300 mL) at 0° C. (ice-water bath) was added 2,6-lutidine (18 mL, 155.4 mmol) followed by trimethylsilyl trifluoromethanesulfonate (15.4 mL, 85.23 mmol) via addition funnel over the course of 12 min. After stirring for 50 min, the reaction was quenched with 1 M aqueous sodium hydroxide (150 mL) and stirred for 10 min. The layers were then separated. The organic layer was washed with 1 M aqueous sodium hydroxide (2×200 mL), dried over magnesium sulfate, filtered, and concentrated to afford (2′S,7R)-2-(2,2-difluoroethyl)-2′-methyl-spiro[4,5-dihydrothieno[2,3-c]pyran-7,4′-piperidine]S44 (23.8 g, 100%) as a viscous pale amber oil. ¹H NMR (300 MHz, Chloroform-d) δ 6.60 (s, 1H), 5.90 (tt, J=56.5, 4.5 Hz, 1H), 3.91 (td, J=5.6, 2.9 Hz, 2H), 3.26 (td, J=16.7, 4.5 Hz, 2H), 3.17-2.99 (m, 2H), 2.92 (ddd, J=12.1, 4.8, 2.0 Hz, 1H), 2.63 (td, J=5.5, 2.9 Hz, 2H), 2.01 (dt, J=13.9, 2.2 Hz, 2H), 1.70 (td, J=13.3, 4.8 Hz, 1H), 1.39 (dd, J=13.6, 11.3 Hz, 2H), 1.06 (d, J=6.4 Hz, 3H). 19F NMR (282 MHz, Chloroform-d) δ −115.07. LCMS m/z 288.1 [M+1]⁺.

### Preparation of S45

### (2′S)-2-(2,2-difluoroethyl)-2′-methyl-1′-prop-2-ynyl-spiro[4,5-dihydrothieno[2,3-c]pyran-7,4′-piperidine](S45)

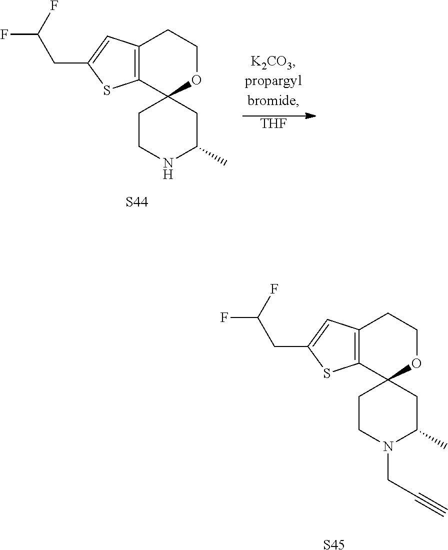 [SMILES: C[C@H]1C[C@@]2(CCN1)OCCc1cc(CC(F)F)sc12 ; "S44" ; C#CCN1CC[C@]2(C[C@@H]1C)OCCc1cc(CC(F)F)sc12 ; "S45" ; "THF"]

### Preparation of (2′S)-2-(2,2-difluoroethyl)-2′-methyl-1′-prop-2-ynyl-spiro[4,5-dihydrothieno[2,3-c]pyran-7,4′-piperidine](S45)

A mixture of (2′S,7R)-2-(2,2-difluoroethyl)-2′-methyl-spiro[4,5-dihydrothieno[2,3-c]pyran-7,4′-piperidine]S44 (700 mg, 2.436 mmol) and potassium carbonate (440 mg, 3.184 mmol) in tetrahydrofuran (14 mL) was heated to 50° C. and stirred. At this time, propargyl bromide (353 μL, 3.961 mmol) in toluene was added and the mixture was stirred at 50° C. overnight. The reaction was quenched with saturated aqueous sodium bicarbonate and DCM. The layers were separated through a phase separator. The organics were concentrated in vacuo. Purification by silica gel chromatography (Gradient: 0-10% MeOH-DCM), yielded (2′S)-2-(2,2-difluoroethyl)-2′-methyl-1′-prop-2-ynyl-spiro[4,5-dihydrothieno[2,3-c]pyran-7,4′-piperidine](S45)(537 mg, 67%) 1H NMR (400 MHz, Methanol-d4) δ 6.64 (s, 1H), 5.96 (tt, J=56.6, 4.3 Hz, 1H), 3.91 (td, J=5.6, 2.2 Hz, 2H), 3.64 (dd, J=17.4, 2.4 Hz, 1H), 3.41 (dd, J=17.3, 2.4 Hz, 1H), 3.25 (ddd, J=17.2, 4.3, 0.9 Hz, 2H), 3.04-2.82 (m, 2H), 2.77-2.54 (m, 4H), 2.13-1.83 (m, 3H), 1.61 (dd, J=13.9, 11.6 Hz, 1H), 1.07 (d, J=6.4 Hz, 3H). LCMS m/z 326.04 [M+1]⁺.

### Preparation of 543

### 1-[4-[[(2′S, 7R)-2-(2,2-difluoroethyl)-2′-methyl-spiro[4,5-dihydrothieno[2,3-c]pyran-7,4′-piperidine]-1′-yl]methyl]triazol-1-yl]propan-2-ol (543)

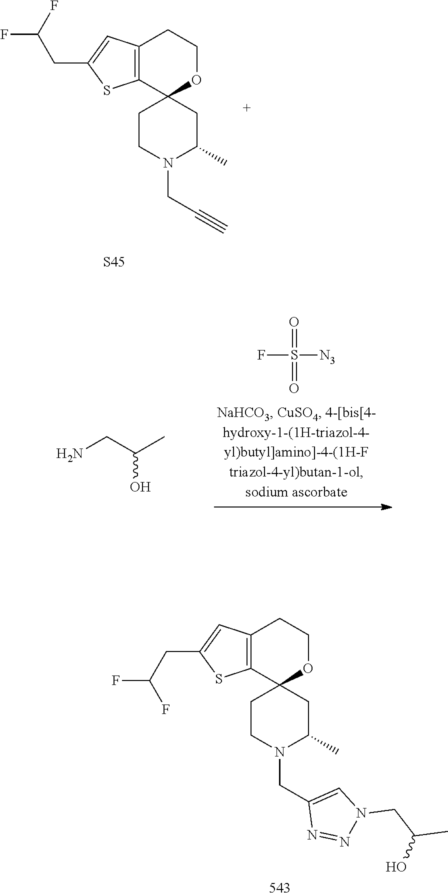

### Preparation of 1-[4-[[(2′S, 7R)-2-(2,2-difluoroethyl)-2′-methyl-spiro[4,5-dihydrothieno[2,3-c]pyran-7,4′-piperidine]-1′-yl]methyl]triazol-1-yl]propan-2-ol (543)

To containing solution of 1-aminopropan-2-ol (20.8 mg, 150 μmol) dissolved in DMSO (0.3 mL) was added aqueous hydrogen carbonate (Sodium salt) (0.25 mL of a 1M solution, 0.25 mmol). To this mixture was added 0.550 mL of fluorosulfonyl azide solution (˜0.45M in MTBE, ˜250 μmol, prepared according the protocol in Nature, 574, 2019, 86-89) and the reaction was stirred at room temperature for 20 minutes. A solution of the of (2′S,7R)-2-(2,2-difluoroethyl)-2′-methyl-1′-prop-2-ynyl-spiro[4,5-dihydrothieno[2,3-c]pyran-7,4′-piperidine]S45 (30 mg, 0.092 mmol) dissolved in DMSO (1.0 mL) was added to the reaction. To this mixture was added a solution of aqueous CuSO₄ (0.085 mL of a 0.1 M solution, 0.0085 mmol), a solution of 4-[bis[4-hydroxy-1-(1H-triazol-4-yl)butyl]amino]-4-(1H-triazol-4-yl)butan-1-ol in DMSO (0.085 mL of a 0.1 M solution, 0.0085 mmol), and an aqueous solution of sodium ascorbate (0.085 mL of a 0.2 M solution, 0.017 mmol). The resulting reaction mixture was heated at 50° C. open to the air overnight. An additional portion of CuSO₄ (0.1 mL of a 0.1 M aq. solution, 0.01 mmol) and sodium ascorbate (0.1 mL of a 0.2 M aq. solution, 0.02 mmol) were added, and the reaction was heated at 50° C. overnight. The reaction was cooled to room temperature, diluted with 2 mL water and 1 mL DCM, and stirred for several minutes. The mixture was passed through a parallel filtration plate and washed with 1 mL DCM. The organic layer was taken and evaporated. The resulting residue was dissolved in 1 mL DMSO and purification by reversed-phase HPLC. Method: C18 Waters Sunfire column (30×150 mm, 5 micron). Gradient: MeCN in H₂O with 10 mM Ammonium Hydroxide to yield 1-[4-[[(2′S,7R)-2-(2,2-difluoroethyl)-2′-methyl-spiro[4,5-dihydrothieno[2,3-c]pyran-7,4′-piperidine]-1′-yl]methyl]triazol-1-yl]propan-2-ol (543) ¹H NMR (300 MHz, Methanol-d4) δ 7.94 (s, 1H), 6.62 (s, 1H), 5.95 (tt, J=56.6, 4.3 Hz, 1H), 4.52-3.67 (m, 7H), 3.24 (dd, J=17.1, 4.3 Hz, 2H), 2.84-2.37 (m, 5H), 2.12-1.78 (i, 3H), 1.75-1.48 (m, 1H), 1.34-0.95 (m, 6H). LCMS m/z 427.16 [M+1]⁺.

### Preparation of Compounds 544-548

Compounds 544-548 (see Table 23) were prepared from intermediate S45 using the appropriate reagents, using the azide transfer and click chemistry methods as described for compound 543. Amines were purchased from commercial sources.

TABLE 23
Method of preparation, structure and physicochemical data for compounds 544-548

| Cmpd | Structure | Amine | ¹H NMR; LCMS m/z [M + H]⁺ |
|---|---|---|---|
| 544 | 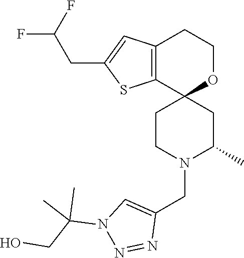 [SMILES: C[C@H]1C[C@@]2(CCN1Cc1cn(C(C)(C)CO)nn1)OCCc1cc(CC(F)F)sc12] | 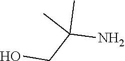 | ¹H NMR (300 MHz, Methanol- d4) δ 8.01 (s, 1H), 6.63 (s, 1H), 5.95 (tt, J = 56.6, 4.3 Hz, 1H), 4.15-3.62 (m, 6H), 3.27-3.14 (m, 1H), 2.82-2.42 (m, 5H), 2.10-1.73 (m, 3H), 1.65 (s, 7H), 1.25 (d, J = 6.2 Hz, 3H). LCMS m/z 441.2 [M + H]⁺ |
| 545 | 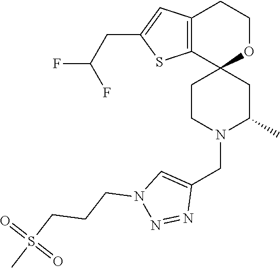 [SMILES: C[C@H]1C[C@@]2(CCN1Cc1cn(CCCS(C)(=O)=O)nn1)OCCc1cc(CC(F)F)sc12] | 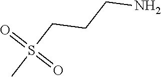 | ¹H NMR (400 MHz, DMSO-d6) δ 8.06 (s, 1H), 6.67 (s, 1H), 6.19 (tt, J = 56.4, 4.3 Hz, 1H), 4.48 (t, J = 7.0 Hz, 2H), 3.86 (d, J = 14.6 Hz, 1H), 3.77 (q, J = 5.4 Hz, 1H), 3.71 (d, J = 14.6 Hz, 1H), 3.42-3.25 (m, 3H), 3.10 (dd, J = 9.3, 6.4 Hz, 2H), 3.00 (s, 3H), 2.59 (d, J = 11.7 Hz, 1H), 2.55- 2.34 (m, 4H), 2.31-2.20 (m, 2H), 1.90 (d, J = 13.3 Hz, 2H), 1.68 (dt, J = 13.9, 7.0 Hz, 1H), 1.44 (t, J = 12.4 Hz, 1H), 1.13 (d, J = 6.1 Hz, 3H). LCMS m/z 489.12 [M + H]⁺ |
| 546 | 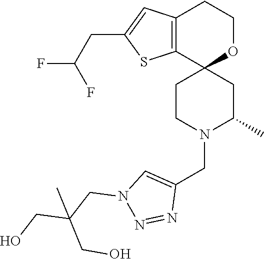 [SMILES: C[C@H]1C[C@@]2(CCN1Cc1cn(CC(C)(CO)CO)nn1)OCCc1cc(CC(F)F)sc12] | 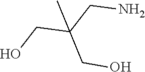 | ¹H NMR (400 MHz, DMSO-d6) δ 7.86 (s, 1H), 6.67 (s, 1H), 6.41- 6.01 (m, 1H), 4.76-4.72 (m, 2H), 4.26 (s, 2H), 3.90-3.61 (m, 4H), 3.32 (s, 2H), 3.25-3.12 (m, 4H), 2.61-2.31 (m, 5H), 1.89 (d, J = 13.0 Hz, 2H), 1.67 (dt, J = 13.3, 6.6 Hz, 1H), 1.49-1.38 (m, 1H), 1.12 (d, J = 6.1 Hz, 3H), 0.66 (s, 3H). LCMS m/z 471.19 [M + H]⁺ |
| 547 | 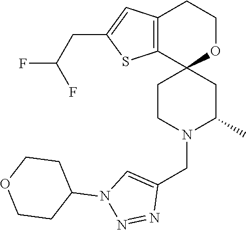 [SMILES: C[C@H]1C[C@@]2(CCN1Cc1cn(C3CCOCC3)nn1)OCCc1cc(CC(F)F)sc12] | 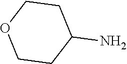 | ¹H NMR (400 MHz, DMSO-d6) δ 8.10 (s, 1H), 6.68 (s, 1H), 6.19 (tt, J = 56.3, 4.2 Hz, 1H), 4.72 (tt, J = 10.1, 4.9 Hz, 1H), 3.96 (d, J = 11.4 Hz, 2H), 3.86 (d, J = 14.5 Hz, 1H), 3.78 (hept, J = 5.6 Hz, 2H), 3.65 (d, J = 14.4 Hz, 1H), 3.49 (td, J = 11.4, 3.0 Hz, 2H), 3.40-3.25 (m, 2H), 2.59 (d, J = 11.9 Hz, 1H), 2.55-2.37 (m, 4H), 2.08-1.96 (m, 4H), 1.90 (d, J = 13.2 Hz, 2H), 1.67 (td, J = 13.1, 4.5 Hz, 1H), 1.44 (t, J = 12.4 Hz, 1H), 1.13 (d, J = 6.1 Hz, 3H). LCMS m/z 453.21 [M + H]⁺ |
| 548 | 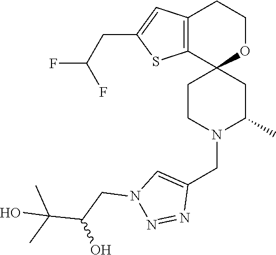 [SMILES: C[C@H]1C[C@@]2(CCN1Cc1cn(CC(O)C(C)(C)O)nn1)OCCc1cc(CC(F)F)sc12] | 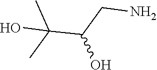 | ¹H NMR (400 MHz, DMSO-d6) δ 7.90 (d, J = 3.5 Hz, 1H), 6.68 (s, 1H), 6.19 (tt, J = 56.4, 4.2 Hz, 1H), 5.13 (d, J = 6.0 Hz, 1H), 4.68-4.56 (m, 2H), 4.12 (dd, J = 13.5, 10.2 Hz, 1H), 3.85 (d, J = 15.2 Hz, 1H), 3.81-3.72 (m, 2H), 3.67 (dd, J = 14.4, 4.4 Hz, 1H), 3.55 (d, J = 7.6 Hz, 1H), 3.42-3.22 (m, 2H), 2.60 (d, J = 11.4 Hz, 1H), 2.54-2.30 (m, 4H), 1.90 (d, J = 13.2 Hz, 2H), 1.71-1.61 (m, 1H), 1.44 (t, J = 12.4 Hz, 1H), 1.16-1.06 (m, 9H). LCMS m/z 471.19 [M + H]⁺ |

### Preparation of S46

### 3-[[(2′S, 7R)-2-(2,2-difluoroethyl)-2′-methyl-spiro[4,5-dihydrothieno[2,3-c]pyran-7,4′-piperidine]-1′-yl]methyl]cyclobutanamine (S46)

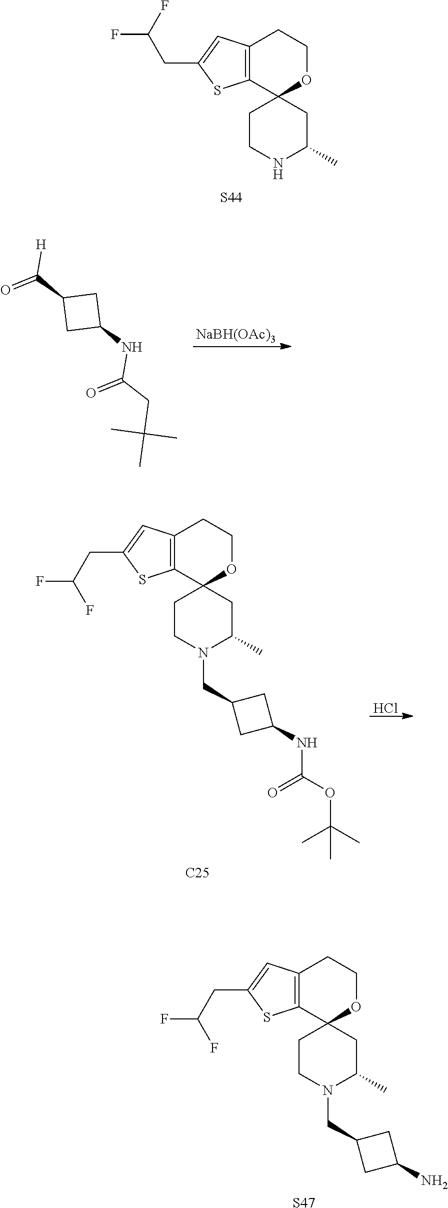

### Step 1. Synthesis of tert-butyl N-[3-[[(2′5,7R)-2-(2,2-difluoroethyl)-2′-methyl-spiro[4,5-dihydrothieno[2,3-c]pyran-7,4′-piperidine]-1′-yl]methyl]cyclobutyl]carbamate (C25)

To a solution of tert-butyl N-(3-formylcyclobutyl)carbamate (177 mg, 0.8883 mmol) and (2′S,7R)-2-(2,2-difluoroethyl)-2′-methyl-spiro[4,5-dihydrothieno[2,3-c]pyran-7,4′-piperidine]S44 (170 mg, 0.5916 mmol) in DCM (5 mL), was added triacetoxyboranuide (Sodium salt) (376 mg, 1.774 mmol). The reaction mixture was stirred at room temperature for 2 hours. The reaction was quenched with NaHCO₃ (sat.), and the mixture was extracted with DCM (3×5 mL). The combined organics were washed with H₂O and brine respectfully and then dried over Na₂SO₄. The solvent was removed in vacuo to afford tert-butyl N-[3-[[(2′S,7R)-2-(2,2-difluoroethyl)-2′-methyl-spiro[4,5-dihydrothieno[2,3-c]pyran-7,4′-piperidine]-1′-yl]methyl]cyclobutyl]carbamate C25 (270 mg, 87%) LCMS m/z 471.68 [M+H]⁺.

### Step 2. Synthesis of 3-[[(2′S,7R)-2-(2,2-difluoroethyl)-2′-methyl-spiro[4,5-dihydrothieno[2,3-c]pyran-7,4′-piperidine]-1′-yl]methyl]cyclobutanamine (S47)

tert-butyl N-[3-[[(2′S,7R)-2-(2,2-difluoroethyl)-2′-methyl-spiro[4,5-dihydrothieno[2,3-c]pyran-7,4′-piperidine]-1′-yl]methyl]cyclobutyl]carbamate C25 was treated with HCl (3 mL of 4 M, 12.00 mmol) in dioxane. The reaction mixture was stirred at room temperature for 1 hour. The solvent was removed to afford 3-[[(2′S,7R)-2-(2,2-difluoroethyl)-2′-methyl-spiro[4,5-dihydrothieno[2,3-c]pyran-7,4′-piperidine]-1′-yl]methyl]cyclobutanamine (189 mg, 83%) LCMS m/z 371.18 [M+H]⁺.

### Compound 549

### 1-cyano-N-((1R,3s)-3-(((2S,4R)-2′-(2,2-difluoroethyl)-2-methyl-4′,5′-dihydrospiro[piperidine-4,7′-thieno[2,3-c]pyran]-1-yl)methyl)cyclobutyl)methanesulfonamide (549)

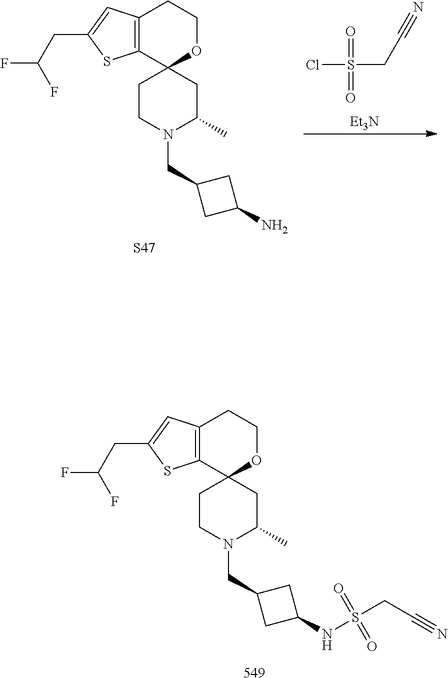 [SMILES: "Et3N" ; C[C@H]1C[C@@]2(CCN1C[C@H]1C[C@@H](N)C1)OCCc1cc(CC(F)F)sc12 ; N#CCS(=O)(=O)Cl ; "S47" ; C[C@H]1C[C@@]2(CCN1C[C@H]1C[C@@H](NS(=O)(=O)CC#N)C1)OCCc1cc(CC(F)F)sc12 ; "549"]

### Synthesis of 1-cyano-N-((1R,3s)-3-(((2S,4R)-2′-(2,2-difluoroethyl)-2-methyl-4′,5′-dihydrospiro[piperidine-4,7′-thieno[2,3-c]pyran]-1-yl)methyl)cyclobutyl)methanesulfonamide (549)

3-[[(2′S,7R)-2-(2,2-difluoroethyl)-2′-methyl-spiro[4,5-dihydrothieno[2,3-c]pyran-7,4′-piperidine]-1′-yl]methyl]cyclobutanamine S47 (20 mg) and cyanomethanesulfonyl chloride (7.2 mg, 0.052 mmol) were mixed in DCM (1 mL), to which, Et₃N (22 uL) was added. The mixture was stirred at room temperature for 2 hours. The solvent was removed. The resulting residue was re-dissolved in MeOH, and purified by reversed-phase HPLC. Method: C18 Waters Sunfire column (30×150 mm, 5 micron). Gradient: MeCN in H2O with 0.1% trifluoroacetic acid to afford 3-[[(2′S,7R)-2-(2,2-difluoroethyl)-2′-methyl-spiro[4,5-dihydrothieno[2,3-c]pyran-7,4′-piperidine]-1′-yl]methyl]-N-oxo-cyclobutanamine (4.7 mg) LCMS m/z 474.02 [M+H]⁺.

### Preparation of Compounds 550-558

Compounds 550-558 (see Table 24) were prepared from intermediate S47 using the appropriate using the appropriate acyl chloride or sulfonyl chloride reagents with the method as described for compound 549. Acyl chlorides or sulfonyl chlorides were purchased from commercial sources.

TABLE 24
Method of preparation, structure and physicochemical data for compounds 550-558

| | | Acyl Chloride or | ¹H NMR; |
|---|---|---|---|
| | | Sulfonyl | LCMS m/z |
| Cmpd | Structure | Chloride | [M + H]⁺ |
| 550 | 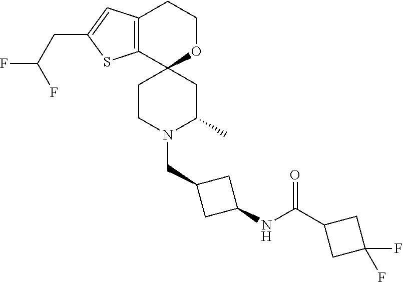 [SMILES: C[C@H]1C[C@@]2(CCN1C[C@H]1C[C@@H](NC(=O)C3CC(F)(F)C3)C1)OCCc1cc(CC(F)F)sc12] | 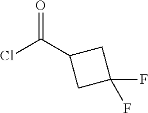 | LCMS m/z 489.13 [M + H]⁺ |
| 551 | 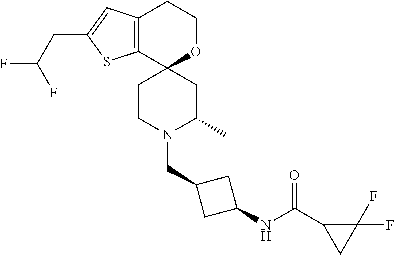 [SMILES: C[C@H]1C[C@@]2(CCN1C[C@H]1C[C@@H](NC(=O)C3CC3(F)F)C1)OCCc1cc(CC(F)F)sc12] | 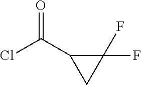 | LCMS m/z 475.16 [M + H]⁺ |
| 552 | 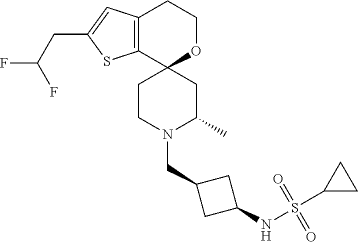 [SMILES: C[C@H]1C[C@@]2(CCN1C[C@H]1C[C@@H](NS(=O)(=O)C3CC3)C1)OCCc1cc(CC(F)F)sc12] | 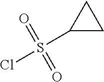 | LCMS m/z 475.13 [M + H]⁺ |
| 553 | 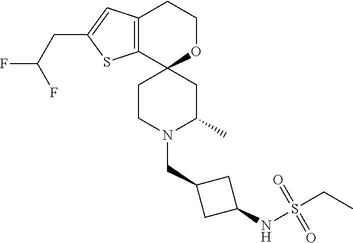 [SMILES: CCS(=O)(=O)N[C@H]1C[C@@H](CN2CC[C@]3(C[C@@H]2C)OCCc2cc(CC(F)F)sc23)C1] | 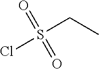 | LCMS m/z 463.11 [M + H]⁺ |
| 554 | 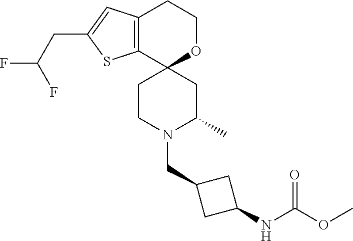 [SMILES: COC(=O)N[C@H]1C[C@@H](CN2CC[C@]3(C[C@@H]2C)OCCc2cc(CC(F)F)sc23)C1] | 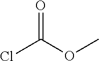 | LCMS m/z 429.16 [M + H]⁺ |
| 555 | 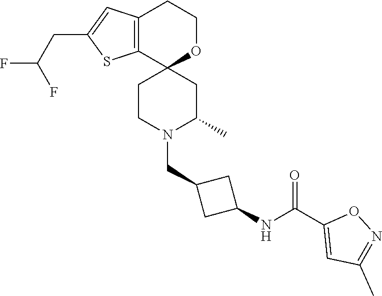 [SMILES: Cc1cc(C(=O)N[C@H]2C[C@@H](CN3CC[C@]4(C[C@@H]3C)OCCc3cc(CC(F)F)sc34)C2)on1] | 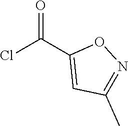 | LCMS m/z 480.14 [M + H]⁺ |
| 556 | 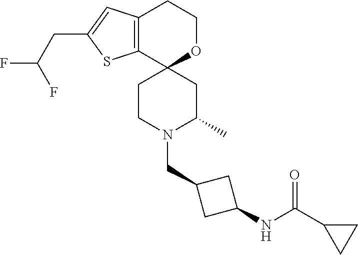 [SMILES: C[C@H]1C[C@@]2(CCN1C[C@H]1C[C@@H](NC(=O)C3CC3)C1)OCCc1cc(CC(F)F)sc12] | 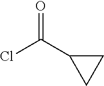 | LCMS m/z 439.13 [M + H]⁺ |
| 557 | 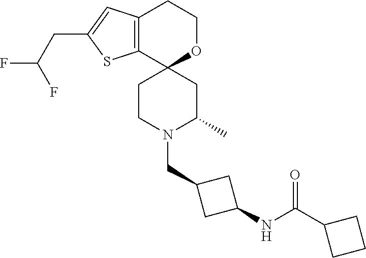 [SMILES: C[C@H]1C[C@@]2(CCN1C[C@H]1C[C@@H](NC(=O)C3CCC3)C1)OCCc1cc(CC(F)F)sc12] | 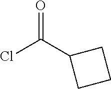 | LCMS m/z 453.2 [M + H]⁺ |
| 558 | 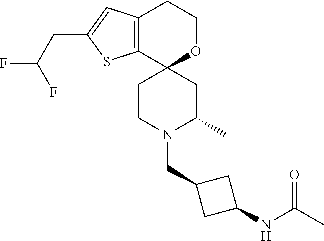 [SMILES: CC(=O)N[C@H]1C[C@@H](CN2CC[C@]3(C[C@@H]2C)OCCc2cc(CC(F)F)sc23)C1] | 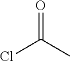 | LCMS m/z 413.17 [M + H]⁺ |

### Compound 559

### (2′S, 7R)-2-(2,2-difluoroethyl)-2′-methyl-1′-[[1-(2-methylsulfonylethyl)pyrazol-4-yl]methyl]spiro[4,5-dihydrothieno[2,3-c]pyran-7,4′-piperidine]

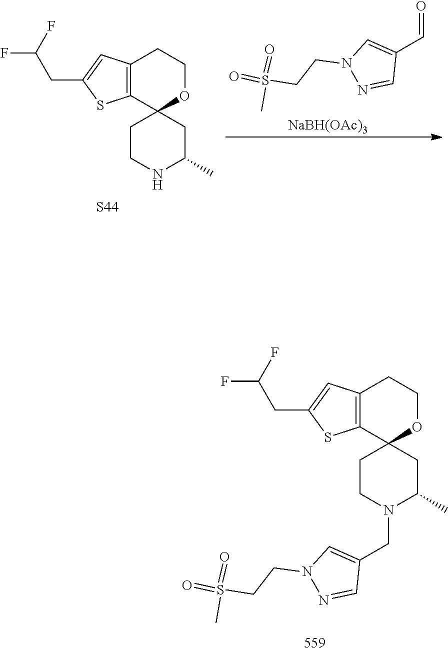 [SMILES: C[C@H]1C[C@@]2(CCN1)OCCc1cc(CC(F)F)sc12 ; CS(=O)(=O)CCn1cc(C=O)cn1 ; "S44" ; C[C@H]1C[C@@]2(CCN1Cc1cnn(CCS(C)(=O)=O)c1)OCCc1cc(CC(F)F)sc12 ; "NaBH(OAc)3"]

### Preparation of (2′S, 7R)-2-(2,2-difluoroethyl)-2′-methyl-1′-[[1-(2-methylsulfonylethyl)pyrazol-4-yl]methyl]spiro[4,5-dihydrothieno[2,3-c]pyran-7,4′-piperidine](559)

(2′S,7R)-2-(2,2-difluoroethyl)-2′-methyl-spiro[4,5-dihydrothieno[2,3-c]pyran-7,4′-piperidine]S44 (295 mg, 0.8864 mmol) and 1-(2-methylsulfonylethyl)pyrazole-4-carbaldehyde (323 mg, 1.597 mmol) were dissolved in THF (4 mL). To this solution was added triacetoxyboranuide (Sodium salt) (470 mg, 2.218 mmol) and the reaction was heated to 60° C. and stirred for 4 hours. The reaction was then cooled to room temperature at which point the reaction was quenched with brine (20 mL) and extracted with EtOAc (20 mL). The organics were dried, filtered, and the solvent was removed under reduced pressure. The resulting residue was purified via reverse phase column chromatography (Isco 150 g gold C-18 gradient 5-95% ACN/water 0.2% FA modifier) to yield (2′S,7R)-2-(2,2-difluoroethyl)-2′-methyl-1′-[[1-(2-methylsulfonylethyl) pyrazol-4-yl]methyl]spiro[4,5-dihydrothieno[2,3-c]pyran-7,4′-piperidine]559 (142.9 mg, 31%) ¹H NMR (400 MHz, Methanol-d4) δ 8.42 (s, 1H), 7.98 (d, J=0.8 Hz, 1H), 7.71 (d, J=0.7 Hz, 1H), 6.69 (d, J=2.6 Hz, 1H), 5.97 (tt, J=56.6, 4.2 Hz, 1H), 4.75-4.65 (m, 2H), 4.46 (d, J=14.1 Hz, 1H), 4.23 (d, J=14.1 Hz, 1H), 3.89 (dt, J=6.2, 5.3 Hz, 2H), 3.76-3.66 (m, 3H), 2.85 (d, J=0.7 Hz, 3H), 2.63 (td, J=5.4, 1.5 Hz, 2H), 2.36-2.13 (m, 3H), 2.13-1.89 (m, 2H), 1.50 (d, J=6.5 Hz, 3H). LCMS m/z 474.38 [M+H]⁺.

### Compound 560

### 2-[[4-[[(2′S, 7R)-2-(2,2-difluoroethyl)-2′-methyl-spiro[4,5-dihydrothieno[2,3-c]pyran-7,4′-piperidine]-1′-yl]methyl]pyrazol-1-yl]methyl]-2-methyl-propane-1,3-diol

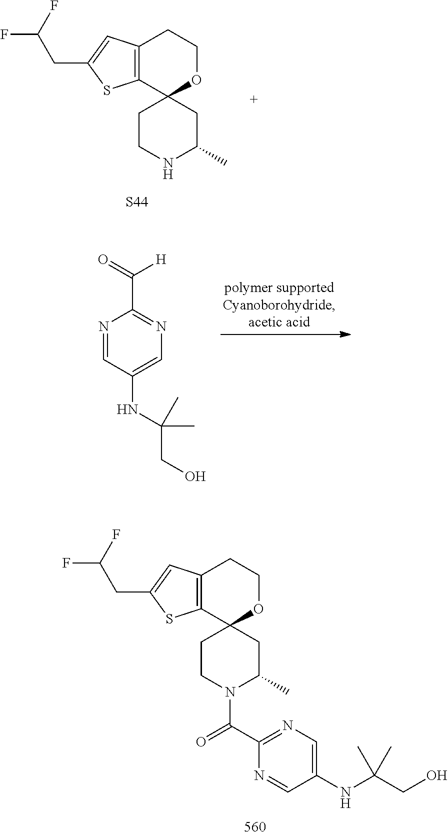 [SMILES: C[C@H]1C[C@@]2(CCN1)OCCc1cc(CC(F)F)sc12 ; "S44" ; CC(C)(CO)Nc1cnc(C=O)nc1 ; C[C@H]1C[C@@]2(CCN1C(=O)c1ncc(NC(C)(C)CO)cn1)OCCc1cc(CC(F)F)sc12 ; "560"]

### Preparation of 2-[[4-[[(2′S, 7R)-2-(2,2-difluoroethyl)-2′-methyl-spiro[4,5-dihydrothieno[2,3-c]pyran-7,4′-piperidine]-1′-yl]methyl]pyrazol-1-ylmethyl]-2-methyl-propane-1, 3-diol (560)

(2′S,7R)-2-(2,2-difluoroethyl)-2′-methyl-spiro[4,5-dihydrothieno[2,3-c]pyran-7,4′-piperidine]S44 (55 mg, 0.19 mmol) was put in a microwave vial 5-((1-hydroxy-2-methylpropan-2-yl)amino)pyrimidine-2-carbaldehyde (37 mg, 0.19 mmol), acetic acid (52 uL, 0.92 mmol), dichloromethane (1.6 mL) were added, and polymer supported cyanoborohydride (295 mg, 0.59 mmol) were added. The solution was capped and heated to 95° C. in a microwave reactor for 120 minutes. MeOH was added and the solution was stirred for ten minutes to wash the beads. The solutions were filtered, and the solvent removed in vacuo. Purification by reversed-phase HPLC. C18 Waters Sunfire column (30×150 mm, 5 micron). Gradient: MeCN in H₂O with 10 mM Ammonium Hydroxide yielded product 2-[[4-[[(2′S,7R)-2-(2,2-difluoroethyl)-2′-methyl-spiro[4,5-dihydrothieno[2,3-c]pyran-7,4′-piperidine]-1′-yl]methyl]pyrazol-1-yl]methyl]-2-methyl-propane-1,3-diol ¹H NMR (300 MHz, Chloroform-d) δ 8.23 (s, 2H), 6.60 (d, J=9.5 Hz, 2H), 5.92 (tt, J=56.5, 4.4 Hz, 1H), 5.24 (s, 1H), 4.05-3.59 (m, 6H), 3.28 (td, J=16.7, 4.5 Hz, 2H), 3.06 (d, J=13.6 Hz, 1H), 2.64 (hept, J=4.4 Hz, 4H), 2.39 (td, J=12.2, 11.7, 2.6 Hz, 1H), 2.01 (ddt, J=14.2, 5.3, 2.8 Hz, 2H), 1.91-1.68 (m, 2H), 1.38 (s, 7H), 1.21 (d, J=6.1 Hz, 4H). LCMS m/z 467.18[M+H]⁺.

### Compound 561

### 2-[[4-[[(2′S, 7R)-2-(2,2-difluoroethyl)-2′-methyl-spiro[4,5-dihydrothieno[2,3-c]pyran-7,4′-piperidine]-1′-yl]methyl]pyrazol-1-yl]methyl]-2-methyl-propane-1,3-diol

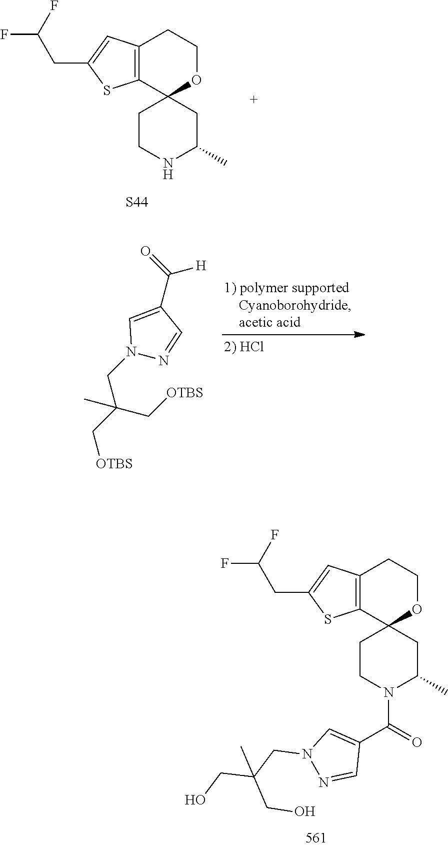 [SMILES: C[C@H]1C[C@@]2(CCN1)OCCc1cc(CC(F)F)sc12 ; "S44" ; CC(CO[Si](C)(C)C(C)(C)C)(CO[Si](C)(C)C(C)(C)C)Cn1cc(C=O)cn1 ; C[C@H]1C[C@@]2(CCN1C(=O)c1cnn(CC(C)(CO)CO)c1)OCCc1cc(CC(F)F)sc12]

### Preparation of 2-[[4-[[(2′S, 7R)-2-(2,2-difluoroethyl)-2′-methyl-spiro[4,5-dihydrothieno[2,3-c]pyran-7,4′-piperidine]-1′-yl]methyl]pyrazol-1-yl]methyl]-2-methyl-propane-1,3-diol (561)

To (2′S,7R)-2-(2,2-difluoroethyl)-2′-methyl-spiro[4,5-dihydrothieno[2,3-c]pyran-7,4′-piperidine]S44 (55 mg, 0.19 mmol) in a microwave via was added 1 1-[3-[tert-butyl(dimethyl)silyl]oxy-2-[[tert-butyl(dimethyl)silyl]oxymethyl]-2-methyl-propyl]pyrazole-4-carbaldehyde (82 mg, 0.19 mmol), acetic acid (52 uL, 0.92 mmol), dichloromethane (1.6 mL), and polymer supported cyanoborohydride (295 mg, 0.59 mmol). The solution was capped and heated to 95° C. in a microwave reactor for 120 minutes. MeOH was added and the solution was stirred for ten minutes to wash the beads. Reaction was stirred with HCl 4M (0.5 mL) in Dioxane for 10 minutes The solutions were filtered and the solvent removed in vacuo. Purification by reversed-phase HPLC. Method: C18 Waters Sunfire column (30×150 mm, 5 micron) (Basic method) yielded product (2′S,7R)-2-(2,2-difluoroethyl)-1′-ethyl-2′-methyl-spiro[4,5-dihydrothieno[2,3-c]pyran-7,4′-piperidine]1H NMR (300 MHz, Chloroform-d) δ 7.46 (s, 1H), 7.37 (s, 1H), 6.61 (s, 1H), 5.92 (tt, J=56.4, 4.5 Hz, 1H), 4.26 (s, 2H), 4.00-3.74 (m, 3H), 3.74-3.06 (m, 9H), 2.86-2.41 (m, 5H), 2.16-1.64 (m, 5H), 1.21 (d, J=6.2 Hz, 3H), 0.84 (s, 3H). ESI-MS m/z 470.24.

### Compounds 562-593

Compounds 562-593 were prepared from the reductive amination steps and in some cases deprotection steps using procedures as described for compounds 559-561 from intermediate S44 and the relevant aldehydes or ketones. Aldehydes or ketones were obtained from commercial sources or synthesized as described above. Any modifications to methods are noted in Table 25 and accompanying footnotes.

TABLE 25
Preparation method, structure and physicochemical data for compounds 562-593.

| | | Aldehyde or | | ¹H NMR; LCMS m/z |
|---|---|---|---|---|
| Cmpd | Structure | Ketone | Method | [M + H]⁺ |
| 562 | 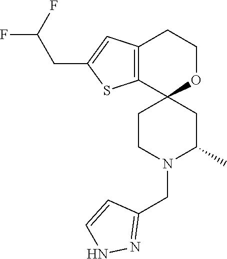 [SMILES: C[C@H]1C[C@@]2(CCN1Cc1cc[nH]n1)OCCc1cc(CC(F)F)sc12] | 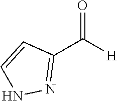 | 560⁴ | ¹H NMR (300 MHz, Methanol-d4) δ 7.59 (s, 2H), 6.62 (s, 1H), 5.95 (tt, J = 56.6, 4.3 Hz, 1H), 4.03-3.71 (m, 3H), 3.65 (d, J = 14.2 Hz, 1H), 3.24 (dd, J = 17.1, 4.3 Hz, 2H), 2.80-2.38 (m, 5H), 2.11-1.56 (m, 4H), 1.23 (d, J = 6.3 Hz, 3H). LCMS m/z 368.19. |
| 563 | 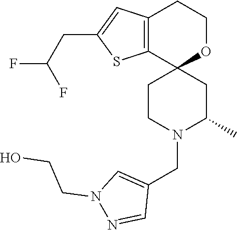 [SMILES: C[C@H]1C[C@@]2(CCN1Cc1cnn(CCO)c1)OCCc1cc(CC(F)F)sc12] | 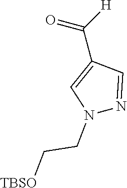 [SMILES: CC(C)(C)[Si](C)(C)OCCn1cc(C=O)cn1] | 561 | ¹H NMR (300 MHz, Methanol-d4) δ 7.55 (dd, J = 49.3, 2.8 Hz, 2H), 6.62 (d, J = 2.8 Hz, 1H), 5.95 (ddt, J = 60.8, 56.7, 4.0 Hz, 1H), 4.21 (dt, J = 8.3, 3.7 Hz, 2H), 3.86 (td, J = 9.9, 8.8, 4.1 Hz, 5H), 3.56 (dd, J = 14.1, 2.9 Hz, 1H), 3.23 (dd, J = 17.1, 4.0 Hz, 2H), 2.85- 2.35 (m, 5H), 2.12- 1.52 (m, 4H), 1.21 (d, J = 5.8 Hz, 3H). LCMS m/z 412.22 |
| 564 | 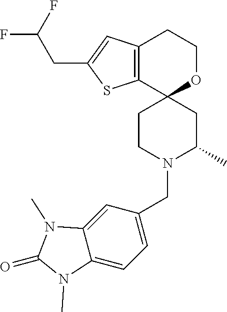 [SMILES: C[C@H]1C[C@@]2(CCN1Cc1ccc3c(c1)n(C)c(=O)n3C)OCCc1cc(CC(F)F)sc12] | 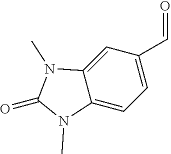 | 560⁴ | ¹H NMR (300 MHz, Chloroform-d) δ 7.19- 6.97 (m, 2H), 6.91 (d, J = 7.9 Hz, 1H), 6.61 (s, 1H), 5.91 (tt, J = 56.5, 4.5 Hz, 1H), 4.25 (d, J = 12.9 Hz, 1H), 3.90 (tdd, J = 11.6, 8.9, 5.1 Hz, 2H), 3.44 (d, J = 8.2 Hz, 6H), 3.38-3.02 (m, 3H), 2.65 (ddt, J = 18.1, 15.9, 8.0 Hz, 4H), 2.35 (td, J = 12.2, 2.7 Hz, 1H), 2.14- 1.68 (m, 4H), 1.25 (d, J = 6.2 Hz, 3H). LCMS m/z 462.18 |
| 565 | 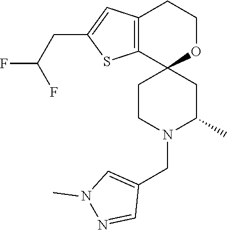 [SMILES: C[C@H]1C[C@@]2(CCN1Cc1cnn(C)c1)OCCc1cc(CC(F)F)sc12] | 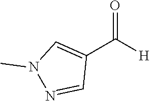 | 559 ^{1,4} | LCMS m/z 382.19 |
| 566 | 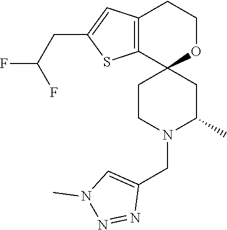 [SMILES: C[C@H]1C[C@@]2(CCN1Cc1cn(C)nn1)OCCc1cc(CC(F)F)sc12] | 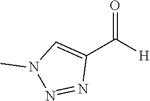 | 559 ^{1,4} | LCMS m/z 383.18 |
| 567 | 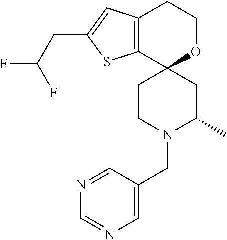 [SMILES: C[C@H]1C[C@@]2(CCN1Cc1cncnc1)OCCc1cc(CC(F)F)sc12] | 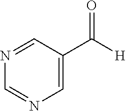 | 559 ^{2,4} | ¹H NMR (300 MHz, Chloroform-d) δ 9.13 (s, 1H), 8.73 (s, 2H), 6.61 (s, 1H), 5.91 (tt, J = 56.5, 4.5 Hz, 1H), 4.21- 4.09 (m, 1H), 4.00-3.81 (m, 2H), 3.40-3.15 (m, 3H), 2.85-2.38 (m, 5H), 2.01 (ddt, J = 16.3, 13.9, 2.9 Hz, 2H), 1.89-1.64 (m, 2H), 1.19 (d, J = 6.2 Hz, 3H). LCMS m/z 380.02 |
| 568 | 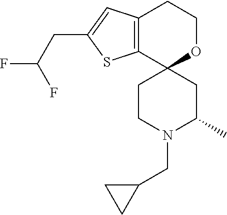 [SMILES: C[C@H]1C[C@@]2(CCN1CC1CC1)OCCc1cc(CC(F)F)sc12] | 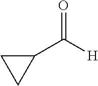 | 559 ^{1,4} | LCMS m/z 342.19 |
| 569 | 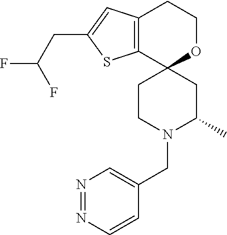 [SMILES: C[C@H]1C[C@@]2(CCN1Cc1ccnnc1)OCCc1cc(CC(F)F)sc12] | 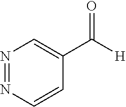 | 559 ^{2,4} | ¹H NMR (300 MHz, Chloroform-d) δ 9.23 (dd, J = 2.3, 1.2 Hz, 1H), 9.12 (dd, J = 5.2, 1.2 Hz, 1H), 7.53 (dd, J = 5.3, 2.2 Hz, 1H), 6.61 (s, 1H), 5.92 (tt, J = 56.5, 4.4 Hz, 1H), 4.20-4.07 (m, 1H), 4.01-3.78 (m, 2H), 3.40-3.15 (m, 3H), 2.87-2.58 (m, 3H), 2.56- 2.42 (m, 2H), 2.08- 1.93 (m, 2H), 1.91-1.80 (m, 1H), 1.79-1.64 (m, 1H), 1.13 (d, J = 6.2 Hz, 3H). LCMS m/z 380.02 |
| 570 | 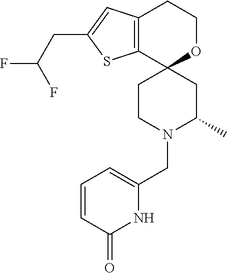 [SMILES: C[C@H]1C[C@@]2(CCN1Cc1cccc(=O)[nH]1)OCCc1cc(CC(F)F)sc12] | 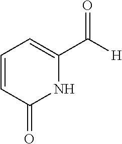 | 559 ² | 1H NMR (300 MHz, Chloroform-d) 8 9.85 (s, 1H), 7.34 (dd, J = 9.2, 6.7 Hz, 1H), 6.62 (s, 1H), 6.44 (d, J = 9.1 Hz, 1H), 6.19-5.70 (m, 2H), 4.04-3.81 (m, 3H), 3.30 (tdd, J = 16.7, 4.5, 0.9 Hz, 2H), 3.14 (d, J = 15.6 Hz, 1H), 2.79 (dtd, J = 12.4, 6.2, 2.5 Hz, 1H), 2.73-2.52 (m, 4H), 2.08-1.82 (m, 3H), 1.67 (dd, J = 14.0, 11.3 Hz, 1H), 1.07 (d, J = 6.2 Hz, 3H). LCMS m/z 395.06 |
| 571 | 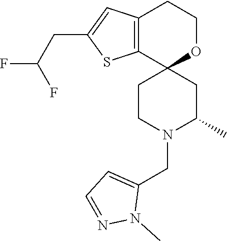 [SMILES: C[C@H]1C[C@@]2(CCN1Cc1ccnn1C)OCCc1cc(CC(F)F)sc12] | 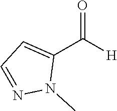 | 559 ^{1,4} | LCMS m/z 382 |
| 572 | 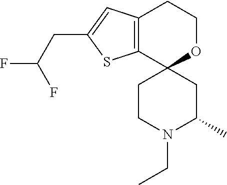 [SMILES: CCN1CC[C@]2(C[C@@H]1C)OCCc1cc(CC(F)F)sc12] | 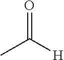 | 559 ^{1,4} | LCMS m/z 316.45 |
| 573 | 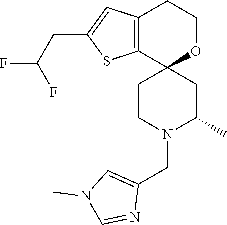 [SMILES: C[C@H]1C[C@@]2(CCN1Cc1cn(C)cn1)OCCc1cc(CC(F)F)sc12] | 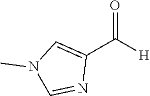 | 559 ^{1,4} | LCMS m/z 382.19 |
| 574 | 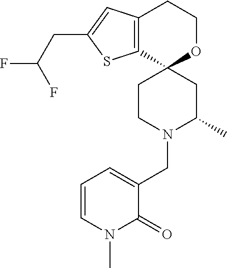 [SMILES: C[C@H]1C[C@@]2(CCN1Cc1cccn(C)c1=O)OCCc1cc(CC(F)F)sc12] | 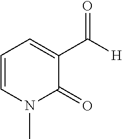 | 559 ^{2,4} | 1H NMR (300 MHz, Chloroform-d) δ 7.57 (d, J = 6.8 Hz, 1H), 7.23 (dd, J = 6.7, 2.1 Hz, 1H), 6.61 (s, 1H), 6.21 (t, J = 6.8 Hz, 1H), 5.92 (tt, J = 56.5, 4.5 Hz, 1H), 3.98- 3.88 (m, 2H), 3.58 (s, 3H), 3.39-3.17 (m, 3H), 2.89-2.49 (m, 5H), 2.12- 1.83 (m, 3H), 1.73 (t, J = 12.4 Hz, 2H), 1.15 (d, J = 6.1 Hz, 3H). LCMS m/z 409.02 |
| 575 | 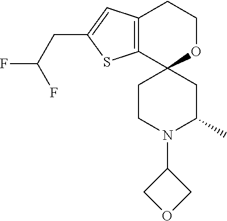 [SMILES: C[C@H]1C[C@@]2(CCN1C1COC1)OCCc1cc(CC(F)F)sc12] | 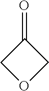 | 559 ^{1,4} | 1H NMR (300 MHz, Acetonitrile-d3) δ 6.68 (s, 1H), 6.35-5.78 (m, 1H), 4.69-4.42 (m, 4H), 3.87 (td, J = 5.6, 1.6 Hz, 2H), 3.67 (p, J = 7.2 Hz, 1H), 3.33 (tdd, J = 17.7, 4.2, 0.9 Hz, 2H), 2.68- 2.54 (m, 2H), 2.44 (dddd, J = 24.8, 12.9, 5.4, 2.6 Hz, 2H), 2.29- 2.11 (m, 2H), 1.94-1.70 (m, 2H), 1.57 (dd, J = 13.7, 11.4 Hz, 1H), 0.85 (d, J = 6.4 Hz, 3H). LCMS m/z 344.59 |
| 576 | 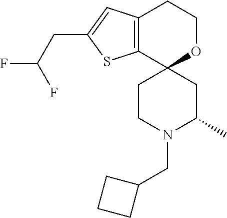 [SMILES: C[C@H]1C[C@@]2(CCN1CC1CCC1)OCCc1cc(CC(F)F)sc12] | 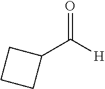 | 559 ^{1,4} | LCMS m/z 356.18 |
| 577 | 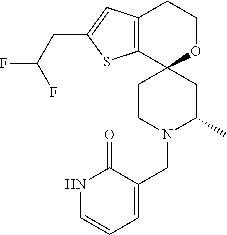 [SMILES: C[C@H]1C[C@@]2(CCN1Cc1ccc[nH]c1=O)OCCc1cc(CC(F)F)sc12] | 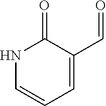 | 559 ^{1,4} | 1H NMR (300 MHz, Acetonitrile-d3) δ 7.48 (dddd, J = 6.5, 5.4, 2.5, 1.6 Hz, 1H), 7.16 (td, J = 6.5, 6.0, 2.1 Hz, 1H), 6.54 (s, 1H), 6.15 (t, J = 6.7 Hz, 1H), 5.90 (tt, J = 56.5, 4.2 Hz, 1H), 3.83- 3.33 (m, 3H), 3.28-3.11 (m, 2H), 3.07 (d, J = 15.5 Hz, 1H), 2.72-2.29 (m, 4H), 1.91-1.84 (m, 1H), 1.79 (d, J = 3.0 Hz, 1H), 1.69 (ddd, J = 13.6, 12.4, 4.5 Hz, 1H), 1.52 (dd, J = 13.7, 11.3 Hz, 1H), 1.12-1.01 (m, 1H), 1.01-0.89 (m, 3H). LCMS m/z 395.06 |
| 578 | 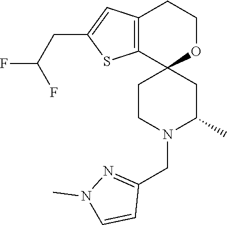 [SMILES: C[C@H]1C[C@@]2(CCN1Cc1ccn(C)n1)OCCc1cc(CC(F)F)sc12] | 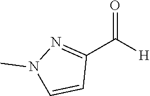 | 559 ^{1,4} | LCMS m/z 382.19 |
| 579 | 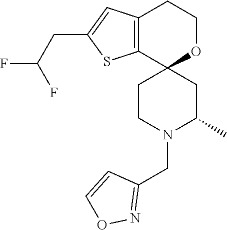 [SMILES: C[C@H]1C[C@@]2(CCN1Cc1ccon1)OCCc1cc(CC(F)F)sc12] | 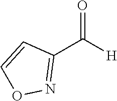 | As per 559 ^{1,4} | LCMS m/z 369.14 |
| 580 | 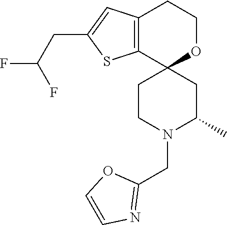 [SMILES: C[C@H]1C[C@@]2(CCN1Cc1ncco1)OCCc1cc(CC(F)F)sc12] | 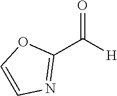 | As per 559 ^{1,4} | LCMS m/z 369.14 |
| 581 | 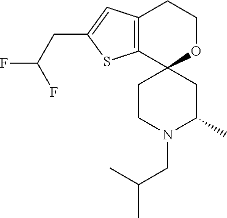 [SMILES: CC(C)CN1CC[C@]2(C[C@@H]1C)OCCc1cc(CC(F)F)sc12] | 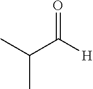 | 559 ^{1,4} | LCMS m/z 344.17 |
| 582 | 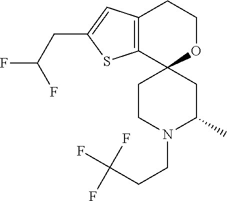 [SMILES: C[C@H]1C[C@@]2(CCN1CCC(F)(F)F)OCCc1cc(CC(F)F)sc12] | 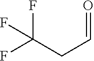 | 559 ^{1,4} | LCMS m/z 384.14 |
| 583 | 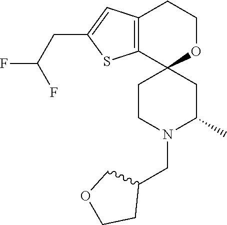 [SMILES: C[C@H]1C[C@@]2(CCN1CC1CCOC1)OCCc1cc(CC(F)F)sc12] | 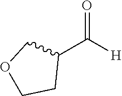 | 559 ^{1,4} | LCMS m/z 372.21 |
| 584 | 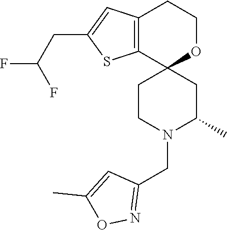 [SMILES: Cc1cc(CN2CC[C@]3(C[C@@H]2C)OCCc2cc(CC(F)F)sc23)no1] | 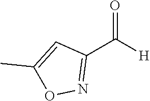 | 559 ^{1,4} | LCMS m/z 383.18 |
| 585 | 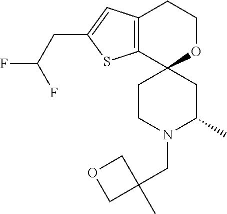 [SMILES: C[C@H]1C[C@@]2(CCN1CC1(C)COC1)OCCc1cc(CC(F)F)sc12] | 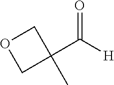 | 559 ^{1,4} | LCMS m/z 372.21 |
| 586 | 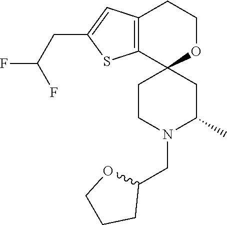 [SMILES: C[C@H]1C[C@@]2(CCN1CC1CCCO1)OCCc1cc(CC(F)F)sc12] | 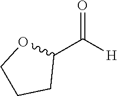 | 559 ^{1,4} | LCMS m/z 372.17 |
| 587 | 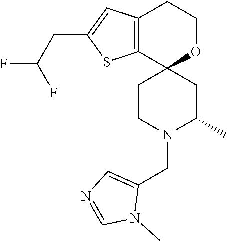 [SMILES: C[C@H]1C[C@@]2(CCN1Cc1cncn1C)OCCc1cc(CC(F)F)sc12] | 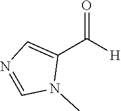 | 559 ^{1,4} | LCMS m/z 382.19 |
| 588 | 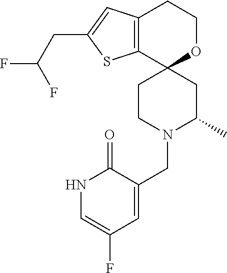 [SMILES: C[C@H]1C[C@@]2(CCN1Cc1cc(F)c[nH]c1=O)OCCc1cc(CC(F)F)sc12] | 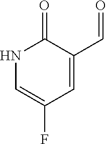 | 559 ^{1,4} | 1H NMR (300 MHz, Chloroform-d) δ 7.68 (d, J = 3.1 Hz, 1H), 7.37 (dd, J = 7.9, 3.1 Hz, 1H), 6.63 (s, 1H), 5.93 (tt, J = 56.4, 4.4 Hz, 1H), 4.30- 4.09 (m, 1H), 4.06-3.81 (m, 2H), 3.42-3.18 (m, 3H), 2.87 (ddt, J = 12.5, 6.2, 3.0 Hz, 1H), 2.80- 2.55 (m, 4H), 2.21-1.86 (m, 3H), 1.75 (dd, J = 14.1, 11.4 Hz, 1H), 1.19 (d, J = 6.2 Hz, 3H). LCMS m/z 412.75 |
| 589 | 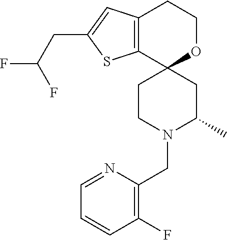 [SMILES: C[C@H]1C[C@@]2(CCN1Cc1ncccc1F)OCCc1cc(CC(F)F)sc12] | 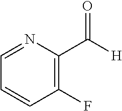 | 559 ^{1,4} | 1H NMR (300 MHz, Chloroform-d) δ 8.46 (dt, J = 4.6, 1.5 Hz, 1H), 7.39 (ddd, J = 9.6, 8.3, 1.4 Hz, 1H), 7.22 (dd, J = 8.4, 4.3 Hz, 1H), 6.60 (s, 1H), 5.91 (tt, J = 56.5, 4.5 Hz, 1H), 4.24 (dd, J = 13.1, 2.7 Hz, 1H), 4.06-3.79 (m, 2H), 3.62 (dd, J = 13.1, 2.5 Hz, 1H), 3.38-3.17 (m, 2H), 2.86-2.49 (m, 5H), 2.07-1.72 (m, 4H), 1.30 (d, J = 6.2 Hz, 3H). LCMS m/z 396.99 |
| 590 | 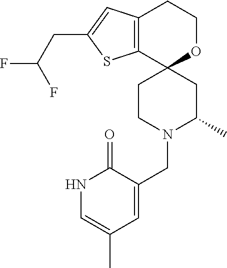 [SMILES: Cc1c[nH]c(=O)c(CN2CC[C@]3(C[C@@H]2C)OCCc2cc(CC(F)F)sc23)c1] | 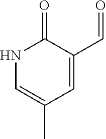 | 559 ^{1,4} | 1H NMR (300 MHz, Chloroform-d) δ 7.43 (d, J = 2.4 Hz, 1H), 7.08 (s, 1H), 6.54 (s, 1H), 5.84 (tt, J = 56.5, 4.5 Hz, 1H), 3.94-3.76 (m, 3H), 3.32- 3.09 (m, 3H), 2.79- 2.42 (m, 5H), 2.07 (d, J = 1.0 Hz, 3H), 2.01- 1.57 (m, 5H), 1.07 (d, J = 6.2 Hz, 3H). LCMS m/z 409.15 |
| 591 | 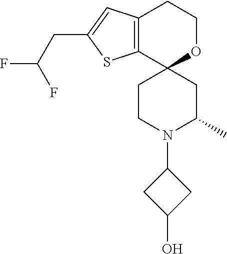 [SMILES: C[C@H]1C[C@@]2(CCN1C1CC(O)C1)OCCc1cc(CC(F)F)sc12] | 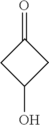 | 559 ^{1,4} | 1H NMR (300 MHz, Chloroform-d) δ 6.61 (s, 1H), 5.92 (tt, J = 56.5, 4.5 Hz, 1H), 4.07-3.82 (m, 3H), 3.27 (tdd, J = 16.6, 4.4, 0.9 Hz, 2H), 2.87-2.74 (m, 1H), 2.70- 2.61 (m, 3H), 2.61- 2.48 (m, 3H), 2.36-2.29 (m, 1H), 2.15-1.99 (m, 2H), 1.98-1.84 (m, 4H), 1.70 (dd, J = 14.0, 11.5 Hz, 1H), 1.08 (d, J = 6.4 Hz, 3H). LCMS m/z 358.59 |
| 592 | 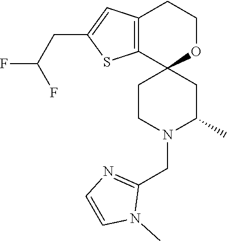 [SMILES: C[C@H]1C[C@@]2(CCN1Cc1nccn1C)OCCc1cc(CC(F)F)sc12] | 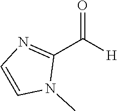 | 559 ^{1,4} | LCMS m/z 382.19 |
| 593 | 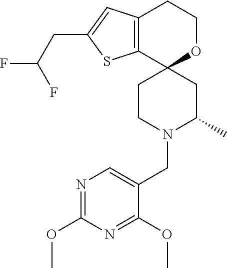 [SMILES: COc1ncc(CN2CC[C@]3(C[C@@H]2C)OCCc2cc(CC(F)F)sc23)c(OC)n1] | 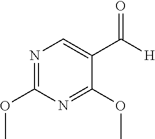 | 559 ^{1,4} | 1H NMR (300 MHz, Chloroform-d) δ 9.78 (s, 1H), 7.35 (dd, J = 9.2, 6.7 Hz, 1H), 6.63 (s, 1H), 6.45 (d, J = 9.2 Hz, 1H), 6.21-5.67 (m, 2H), 4.02-3.82 (m, 2H), 3.43- 3.21 (m, 2H), 3.15 (d, J = 15.6 Hz, 1H), 2.81 (ddd, J = 11.4, 6.2, 2.4 Hz, 1H), 2.74-2.48 (m, 4H), 2.12-1.83 (m, 3H), 1.68 (dd, J = 14.0, 11.3 Hz, 1H), 1.38-1.16 (m, 1H), 1.07 (d, J = 6.2 Hz, 3H). LCMS m/z 440.03 |

Footnotes:
^{1.}DCM was used in place of THF as the solvent and the reaction was run at room temperature for 2 hours.
^{2.}DCM was used in place of THE as the solvent and Et₃N (1 equiv.) was used in addition to the standard reagents. The reaction was stirred at 50 C. for 30 min. prior to the addition of the sodium triacetoxytborhydride which was added to the reaction after cooling to room temperature and stirred overnight before the reaction was quenched. Additionally, the compound was purified via silica gel (Redi-Sep cartridge, 4 g), eluting with 0-10% DCM in MeOH (with 7N NH₃) to afford product.
^{3.}DCM was used in place of THE as the solvent and DIPEA (1 equiv.) was used in addition to the standard reagents. The reaction was stirred at reflux for 30 min. prior to the addition of the sodium triacetoxytborhydride which was added to the reaction after cooling to room temperature and stirred overnight before the reaction was quenched. Additionally, the compound was purified via silica gel (Redi-Sep cartridge, 4 g), eluting with 0-10% DCM in MeOH (with 7N NH₃) to afford product.
^{4.}TBS deprotection with HCl was not used.

### Compound 594

### 2-[(2′S, 7R)-2-(2,2-difluoroethyl)-2′-methyl-spiro[4,5-dihydrothieno[2,3-c]pyran-7,4′-piperidine]-1′-yl]-1-phenyl-ethanol (594)

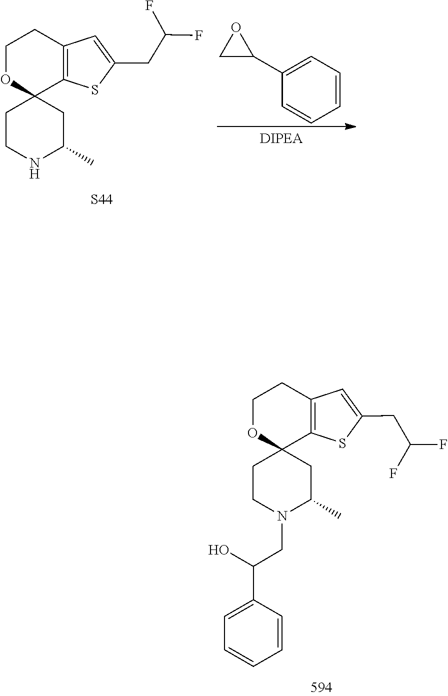

### Preparation of 2-[(2′S, 7R)-2-(2,2-difluoroethyl)-2′-methyl-spiro[4,5-dihydrothieno[2,3-c]pyran-7,4′-piperidine]-1′-yl]-1-phenyl-ethanol (594)

To a solution of (2′S,7R)-2-(2,2-difluoroethyl)-2′-methyl-spiro[4,5-dihydrothieno[2,3-c]pyran-7,4′-piperidine](Hydrochloride salt) (9.8 mg, 0.02875 mmol) in methanol (1 ml) was added Hunig's base (40 μL, 0.2296 mmol) and 2-phenyloxirane (10 μL, 0.08748 mmol) in a microwave vial. Reaction was run in the microwave for 15 minutes at 100° C. Solvent was evaporated. Purification by reversed-phase HPLC. Method: C18 Waters Sunfire column (30×150 mm, 5 micron). Gradient: MeCN in H2O with 0.2% formic acid afforded 2-[(2′S,7R)-2-(2,2-difluoroethyl)-2′-methyl-spiro[4,5-dihydrothieno[2,3-c]pyran-7,4′-piperidine]-1′-yl]-1-phenyl-ethanol 594 (6.5 mg, 55%) LCMS m/z 408.16 [M+1]⁺.

### Compounds 595-609

Compounds 595-609 and intermediates S48 and S49 were prepared from an epoxide opening step as described for compound 594 from intermediate S44 and the relevant epoxides. Epoxides were obtained from commercial sources. Any modifications to methods are noted in Table 26 and accompanying footnotes.

TABLE 26
Method of preparation, structure and physicochemical data for compounds 595-
609 and intermediates S48 and S49

| | | | | ¹H NMR; LCMS m/z |
|---|---|---|---|---|
| Cmpd | Structure | Epoxide | Method | [M + H]⁺ |
| 595 | 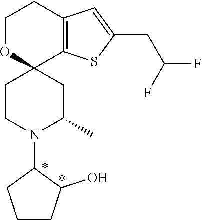 [SMILES: C[C@H]1C[C@@]2(CCN1C1CCCC1O)OCCc1cc(CC(F)F)sc12] | 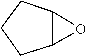 | Compound 594^{1,2,3,5,6} | ¹H NMR (300 MHZ, Chloroform-d) δ 8.52 (s, 1H), 6.64 (s, 1H), 5.92 (tt, J = 56.4, 4.4 Hz, 1H), 4.55 (q, J = 6.5 Hz, 1H), 4.02- 3.71 (m, 3H), 3.53- 3.07 (m, 4H), 2.79- 2.61 (m, 2H), 2.48 (tt, J = 13.2, 2.8 Hz, 2H), 2.31-1.63 (m, 6H), 1.45 (d, J = 6.5 Hz, 3H). LCMS m/z 372.21 [M + 1]⁺ |
| 596 | 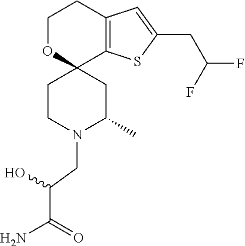 [SMILES: C[C@H]1C[C@@]2(CCN1CC(O)C(N)=O)OCCc1cc(CC(F)F)sc12] | 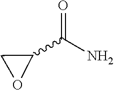 | Compound 594 | LCMS m/z 375.15 [M + 1]⁺ |
| 597 | 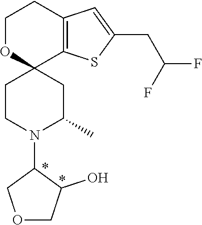 [SMILES: C[C@H]1C[C@@]2(CCN1C1COCC1O)OCCc1cc(CC(F)F)sc12] | 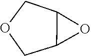 | Compound 594^{1,2,4,6} | ¹H NMR (300 MHZ, Chloroform-d) δ 6.62 (s, 1H), 5.92 (tt, J = 56.5, 4.5 Hz, 1H), 4.67 (ddd, J = 5.4, 3.4, 1.7 Hz, 1H), 4.24-4.00 (m, 2H), 4.00-3.78 (m, 4H), 3.68 (dd, J = 10.0, 3.4 Hz, 1H), 3.27 (tdd, J = 16.7, 4.5, 0.9 Hz, 2H), 2.98 (dtd, J = 12.4, 6.2, 2.4 Hz, 1H), 2.87 (ddd, J = 11.6, 4.4, 2.6 Hz, 1H), 2.71- |
| | | | | 2.41 (m, 3H), 2.06 |
| | | | | (ddt, J = 14.2, 5.7, 2.9 |
| | | | | Hz, 2H), 1.95-1.77 |
| | | | | (m, 2H), 1.69 (dd, J = |
| | | | | 13.9, 11.3 Hz, 1H), |
| | | | | 1.23 (d, J = 6.3 Hz, |
| | | | | 3H). LCMS m/z 374.2 |
| | | | | [M + 1]⁺ |
| 598 | 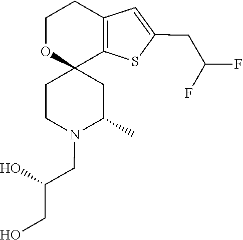 [SMILES: C[C@H]1C[C@@]2(CCN1C[C@@H](O)CO)OCCc1cc(CC(F)F)sc12] | 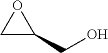 | Compound 594 | LCMS m/z 362.19 [M + 1]⁺ |
| 599 | 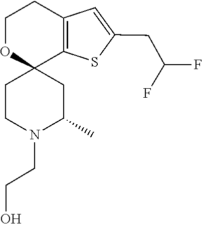 | 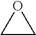 | Compound 594 | LCMS m/z 332.16 [M + 1]⁺ |
| 600 | 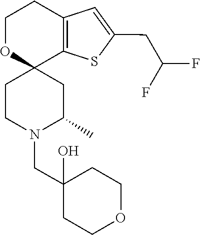 [SMILES: C[C@H]1C[C@@]2(CCN1CC1(O)CCOCC1)OCCc1cc(CC(F)F)sc12] | 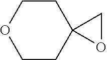 | Compound 594 | LCMS m/z 402.19 [M + 1]⁺ |
| 601 | 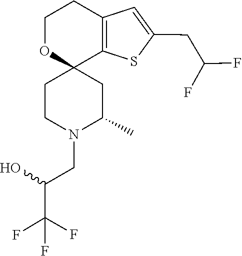 [SMILES: C[C@H]1C[C@@]2(CCN1CC(O)C(F)(F)F)OCCc1cc(CC(F)F)sc12] | 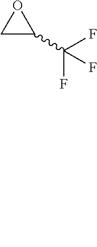 | Compound 594 | LCMS m/z 400.12 [M + 1]⁺ |
| 602 | 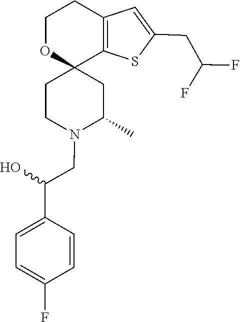 [SMILES: C[C@H]1C[C@@]2(CCN1CC(O)c1ccc(F)cc1)OCCc1cc(CC(F)F)sc12] | 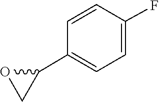 | Compound 594 | LCMS m/z 426.17 [M + 1]⁺ |
| 603 | 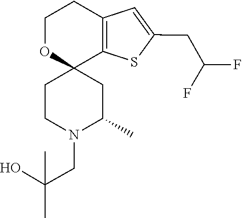 [SMILES: C[C@H]1C[C@@]2(CCN1CC(C)(C)O)OCCc1cc(CC(F)F)sc12] | 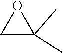 | Compound 594 | LCMS m/z 360.2 [M + 1]⁺ |
| 604 | 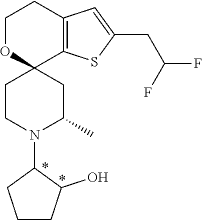 [SMILES: C[C@H]1C[C@@]2(CCN1C1CCCC1O)OCCc1cc(CC(F)F)sc12] | 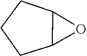 | Compound 594^{1,2,3,6} | ¹H NMR (300 MHZ, Chloroform-d) δ 6.63 (s, 1H), 5.92 (tt, J = 56.4, 4.4 Hz, 1H), 4.56 (d, J = 6.7 Hz, 1H), 4.04-3.84 (m, 2H), 3.71 (d, J = 7.3 Hz, 1H), 3.48 (s, 1H), 3.28 (tdd, J = 16.5, 4.5, 0.9 Hz, 2H), 3.12 (s, 2H), 2.67 (td, J = 5.4, 1.7 Hz, 2H), 2.13 (d, J = 12.1 Hz, 4H), 2.04- |
| | | | | 1.59 (m, 4H), 1.40 |
| | | | | (d, J = 6.2 Hz, |
| | | | | 3H). LCMS m/z |
| | | | | 372.21 [M + 1]⁺ |
| 605 | 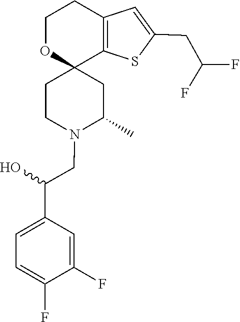 [SMILES: C[C@H]1C[C@@]2(CCN1CC(O)c1ccc(F)c(F)c1)OCCc1cc(CC(F)F)sc12] | 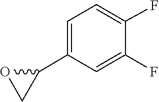 [SMILES: Fc1ccc(C2CO2)cc1F] | Compound 594 | LCMS m/z 444.14 [M + 1]⁺ |
| 606 | 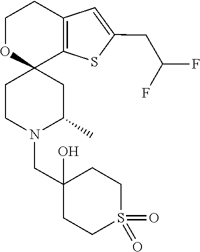 [SMILES: C[C@H]1C[C@@]2(CCN1CC1(O)CCS(=O)(=O)CC1)OCCc1cc(CC(F)F)sc12] | 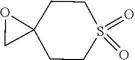 | Compound 594 | LCMS m/z 450.15 [M + 1]⁺ |
| 607 | 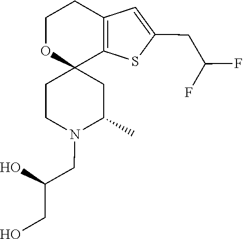 [SMILES: C[C@H]1C[C@@]2(CCN1C[C@H](O)CO)OCCc1cc(CC(F)F)sc12] | 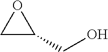 | Compound 594 | LCMS m/z 362.19 [M + 1]⁺ |
| 608 | 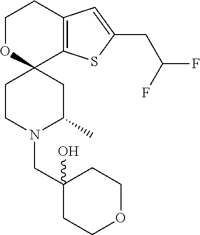 [SMILES: C[C@H]1C[C@@]2(CCN1CC1(O)CCOCC1)OCCc1cc(CC(F)F)sc12] | 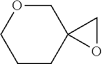 | Compound 594 | LCMS m/z 402.19 [M + 1]⁺ |
| 609 | 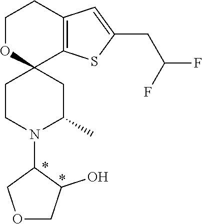 [SMILES: C[C@H]1C[C@@]2(CCN1C1COCC1O)OCCc1cc(CC(F)F)sc12] | 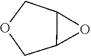 | Compound 594^{1,2,4,6} | 1H NMR (300 MHZ, Chloroform-d) δ 6.62 (s, 1H), 5.92 (tt, J = 56.5, 4.5 Hz, 1H), 4.50 (ddd, J = 6.4, 5.1, 2.9 Hz, 1H), 4.11 (ddd, J = 11.3, 9.6, 5.3 Hz, 2H), 4.01-3.83 (m, 2H), 3.78 (dd, J = 9.6, 6.8 Hz, 1H), 3.74-3.55 (m, 2H), 3.40-3.19 (m, 2H), 2.93-2.76 (m, 2H), 2.72-2.50 |
| | | | | (m, 3H), 2.20-1.59 |
| | | | | (m, 4H), 1.15 (d, J = |
| | | | | 6.3 Hz, 3H). LCMS |
| | | | | m/z 374.2 [M + 1]⁺ |
| S48 | 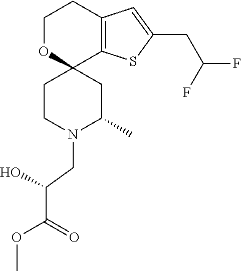 [SMILES: COC(=O)[C@H](O)CN1CC[C@]2(C[C@@H]1C)OCCc1cc(CC(F)F)sc12] | 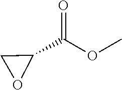 | Compound 594^{7,8} | LCMS m/z 390.13 [M + 1]⁺ |
| S49 | 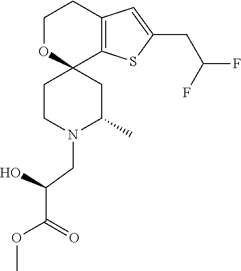 [SMILES: COC(=O)[C@@H](O)CN1CC[C@]2(C[C@@H]1C)OCCc1cc(CC(F)F)sc12] | 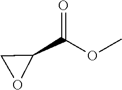 | Compound 594^{7,8} | LCMS m/z 390.13 [M + 1]⁺ |

Footnotes:
¹IPA (1 mL) was used as the solvent,
²Additional 3 eq. epoxide was added after heating at 150º C. for two hours.
³Reaction was heated for 150º C. for eight hours in the microwave
⁴Product was repurified by silica gel chromatography (Gradient: 0-20% MeOH in DCM)
⁵Product was submitted as a formic acid salt
⁶Two diastereomers were separated during purification
⁷Reaction was heated for 90º C. for 1.5 hours in the microwave
⁸After reaction had gone to completion solvent was removed in vacuo, rinsed with MeOH and DCM and the crude reaction was telescoped to the next reaction.

### Compound 610

### (2R)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-propanamide (610)

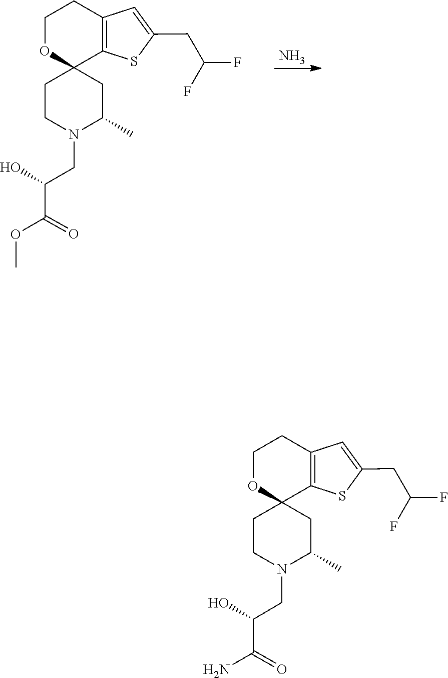

### Preparation of (2R)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-propanamide (610)

Methyl (2R)-3-[(2′S,7R)-2-(2,2-difluoroethyl)-2′-methyl-spiro[4,5-dihydrothieno[2,3-c]pyran-7,4′-piperidine]-1′-yl]-2-hydroxy-propanoate S48 (96 mg, 0.2465 mmol) was added to a microwave flask. A solution of NH₃ (1.5 mL of 7 M, 10.50 mmol) in MeOH (100 μL) was added and the reaction was stirred at 50° C. overnight. Solvent was removed in vacuo. Purification by Silica Gel Chromotography (Gradient: 0-10% MeOH in DCM) provided the desired product 610. ¹H NMR (300 MHz, Chloroform-d) δ 6.94 (d, J=4.2 Hz, 1H), 6.58 (s, 1H), 6.13-5.61 (m, 2H), 3.93-3.76 (m, 2H), 3.24 (td, J=16.8, 4.4 Hz, 2H), 2.91 (dd, J=12.9, 9.9 Hz, 1H), 2.80 (ddq, J=11.5, 7.0, 2.5 Hz, 2H), 2.62 (tdd, J=13.1, 10.3, 3.7 Hz, 4H), 1.99-1.86 (m, 2H), 1.86-1.69 (m, 1H), 1.56 (dd, J=14.0, 11.4 Hz, 1H), 1.03 (d, J=6.2 Hz, 3H). LCMS m/z 375.14 [M+1]⁺.

### Compound 611

### (2S)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-propanamide (611)

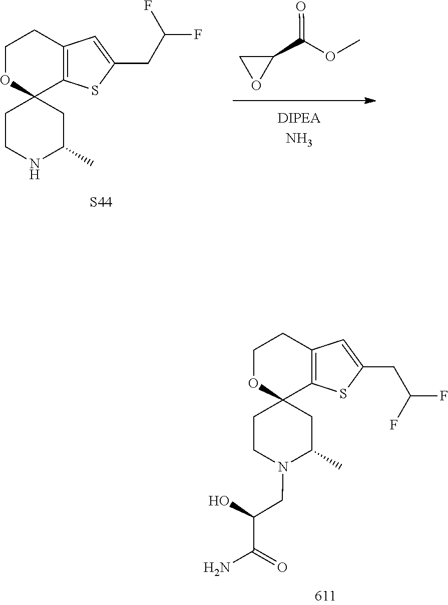

### Preparation of (2S)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-propanamide (611)

(2′S,7R)-2-(2,2-difluoroethyl)-2′-methyl-spiro[4,5-dihydrothieno[2,3-c]pyran-7,4′-piperidine]S44 (108 mg, 0.3758 mmol) and methyl (2S)-oxirane-2-carboxylate (700 μL, 7.995 mmol) were added to a microwave vial and brought up in NH₃ (4 mL of 7 M, 28.00 mmol) in MeOH. DIPEA (980 μL, 5.626 mmol) was added and reaction stirred at 120° C. for 6 hours. Solvent was removed in vacuo and product was rinsed with MeOH and DCM (2×). Purification by reversed-phase HPLC. Method: C18 Waters Sunfire column (30×150 mm, 5 micron) followed by Silica gel chromatography (Gradient: 0-20% MeOH in dichloromethane) yielded the product (2S)-3-[(2′S,7R)-2-(2,2-difluoroethyl)-2′-methyl-spiro[4,5-dihydrothieno[2,3-c]pyran-7,4′-piperidine]-1′-yl]-2-hydroxy-propanamide 611 (17.8 mg, 12%). ¹H NMR (300 MHz, Chloroform-d) δ 7.29 (s, 1H), 6.61 (s, 1H), 6.11-5.66 (m, 2H), 4.38 (s, 1H), 3.87 (hept, J=5.9 Hz, 2H), 3.61 (s, 1H), 3.26 (td, J=16.7, 4.4 Hz, 4H), 3.11 (s, 1H), 2.77 (dd, J=13.5, 8.2 Hz, 1H), 2.63 (q, J=5.2 Hz, 2H), 1.29 (d, J=6.4 Hz, 3H). LCMS m/z 375.14 [M+1]⁺.

### Compound 612

### (2S)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-N-methyl-propanamide (612)

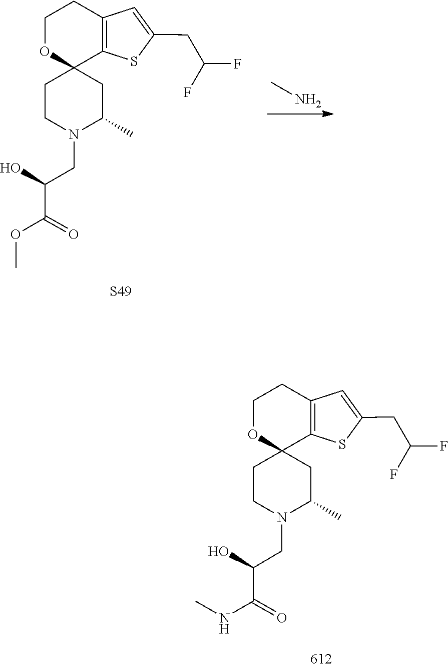

### Preparation of (2S)-3-[(2′S, 7R)-2-(2,2-difluoroethyl)-2′-methyl-spiro[4,5-dihydrothieno[2,3-c]pyran-7,4′-piperidine]-1′-yl]-2-hydroxy-N-methyl-propanamide (612)

Methyl (S)-3-((2S,4R)-2′-(2,2-difluoroethyl)-2-methyl-4′,5′-dihydrospiro[piperidine-4,7′-thieno[2,3-c]pyran]-1-yl)-2-hydroxypropanoate 49 (95 mg, 0.2439 mmol) was brought up in dioxane (1.9 mL) and added to a microwave flask. Methyl amine (250 μL of 40% w/v, 3.220 mmol) and water (45 μL, 2.498 mmol) were added and the reaction was heated to 80° C. and stirred overnight. Solvent was removed in vacuo. Purification by reversed-phase HPLC. Method: C18 Waters Sunfire column (30×150 mm, 5 micron). Gradient: MeCN in H2O with 0.1% trifluoroacetic acid yielded (2S)-3-[(2′S,7R)-2-(2,2-difluoroethyl)-2′-methyl-spiro[4,5-dihydrothieno[2,3-c]pyran-7,4′-piperidine]-1′-yl]-2-hydroxy-N-methyl-propanamide (Trifluoroacetic Acid (2)) 612 (60.1 mg, 39%). ¹H NMR (300 MHz, Chloroform-d) δ 7.41 (d, J=5.8 Hz, 1H), 6.64 (d, J=4.2 Hz, 1H), 5.91 (tt, J=56.3, 4.4 Hz, 1H), 4.81-4.46 (m, 1H), 4.02 (s, OH), 3.89 (dp, J=10.6, 5.9 Hz, 2H), 3.68 (d, J=12.1 Hz, 1H), 3.53 (s, 1H), 3.27 (td, J=16.7, 4.4 Hz, 3H), 3.13-2.92 (m, 1H), 2.86 (d, J=4.9 Hz, 2H), 2.71-2.60 (m, 2H), 2.46-2.26 (m, 1H), 2.26-2.07 (m, 2H), 1.48 (d, J=6.4 Hz, 2H). LCMS m/z 389.28 [M+1]⁺.

### Compound 613

### 1-((2S,4R)-2′-(2,2-difluoroethyl)-2-methyl-4′,5′-dihydrospiro[piperidine-4,7′-thieno[2,3-c]pyran]-1-yl)propan-2-ol (613)

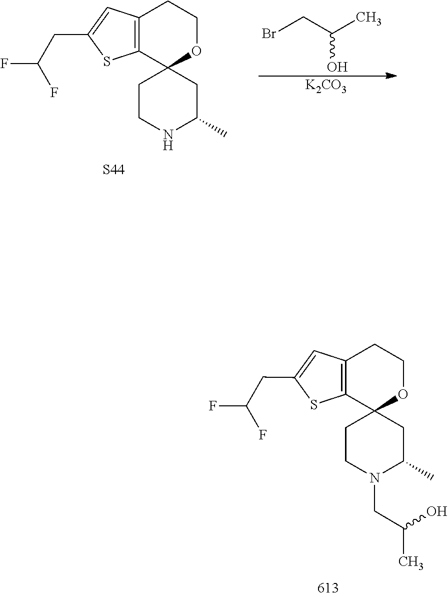

### Preparation of 1-((2S,4R)-2′-(2,2-difluoroethyl)-2-methyl-4′,5′-dihydrospiro[piperidine-4,7′-thieno[2,3-c]pyran]-1-yl)propan-2-ol (613)

To a solution of 1-bromopropan-2-ol (16.51 mg, 0.1188 mmol) and (2′S,7R)-2-(2,2-difluoroethyl)-2′-methyl-spiro[4,5-dihydrothieno[2,3-c]pyran-7,4′-piperidine](Hydrochloride salt) (27 mg, 0.07921 mmol) in DMF (2 mL) was added K₂CO₃ (32.84 mg, 0.2376 mmol). The reaction was stirred at 110° C. for 72 hours, then cooled to room temperature and filtered. The crude reaction mixture was purified by reverse phase HPLC (Method: C18 Waters Sunfire column (30×150 mm, 5 micron). Gradient: MeCN in H₂O with 0.1% trifluoroacetic acid) to afford 1-((2S,4R)-2′-(2,2-difluoroethyl)-2-methyl-4′,5′-dihydrospiro[piperidine-4,7′-thieno[2,3-c]pyran]-1-yl)propan-2-ol (613) (8.8 mg, 31.5%). 1H NMR (300 MHz, Acetonitrile-d3) δ 6.68 (d, J=1.1 Hz, 1H), 6.04 (tt, J=56.5, 4.2 Hz, 1H), 3.89 (tt, J=5.7, 1.8 Hz, 2H), 3.84-3.63 (m, 1H), 3.33 (tdt, J=17.7, 4.2, 0.8 Hz, 3H), 2.91-2.36 (m, 6H), 2.14-2.03 (m, 1H), 2.03-1.98 (m, 1H), 1.93-1.77 (m, 2H), 1.56 (ddd, J=20.6, 13.7, 11.3 Hz, 1H), 1.13-0.97 (m, 6H). LCMS m/z 345.44 [M+H]⁺.

### Compounds 614-624

Compounds 614-624 (Table 27) were prepared from intermediate S44 and the appropriate amine reagent using the method for compound 613 Alkyl halides were obtained from commercial sources. Any modifications to methods are noted in Table 27 and accompanying footnotes.

TABLE 27
Structure and physicochemical data for compounds 614-624

| | | | ¹H NMR; LCMS |
|---|---|---|---|
| Compd | Structure | Alkyl halide | m/z [M + H]⁺ |
| 614 | 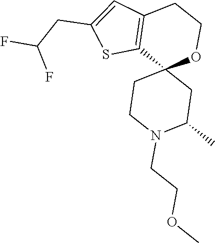 [SMILES: COCCN1CC[C@]2(C[C@@H]1C)OCCc1cc(CC(F)F)sc12] | 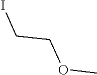 | LCMS m/z 346.2 [M + H]⁺. |
| 615 | 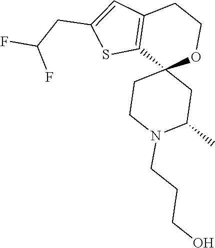 [SMILES: C[C@H]1C[C@@]2(CCN1CCCO)OCCc1cc(CC(F)F)sc12] | 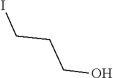 | LCMS m/z 346.2 [M + H]⁺. |
| 616 | 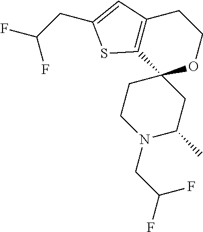 [SMILES: C[C@H]1C[C@@]2(CCN1CC(F)F)OCCc1cc(CC(F)F)sc12] | 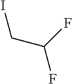 | LCMS m/z 352.17 [M + H]⁺. |
| 617 | 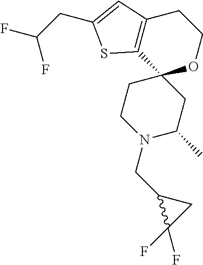 [SMILES: C[C@H]1C[C@@]2(CCN1CC1CC1(F)F)OCCc1cc(CC(F)F)sc12] | 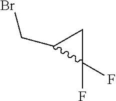 | LCMS m/z 378.15 [M + H]⁺.¹ |
| 618 | 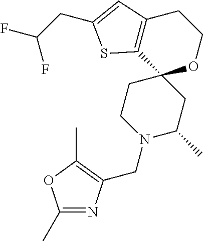 [SMILES: Cc1nc(CN2CC[C@]3(C[C@@H]2C)OCCc2cc(CC(F)F)sc23)c(C)o1] | 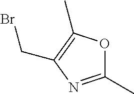 | LCMS m/z 397.17 [M + H]⁺.¹ |
| 619 | 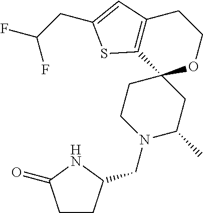 | 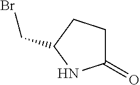 | LCMS m/z 385.02 [M + H]⁺.¹ |
| 620 | 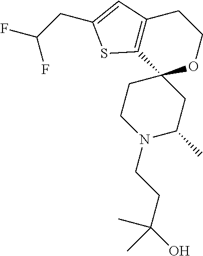 [SMILES: C[C@H]1C[C@@]2(CCN1CCC(C)(C)O)OCCc1cc(CC(F)F)sc12] | 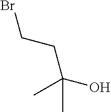 | LCMS m/z 374.18 [M + H]⁺.¹ |
| 621 | 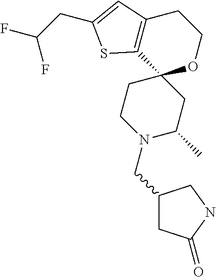 [SMILES: C[C@H]1C[C@@]2(CCN1CC1CNC(=O)C1)OCCc1cc(CC(F)F)sc12] | 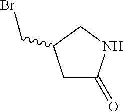 | LCMS m/z 385.15 [M + H]⁺.¹ |
| 622 | 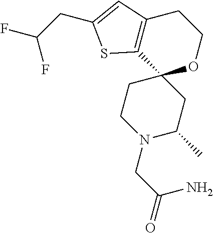 [SMILES: C[C@H]1C[C@@]2(CCN1CC(N)=O)OCCc1cc(CC(F)F)sc12] | 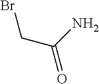 | LCMS m/z 345.12 [M + H]⁺.¹ |
| 623 | 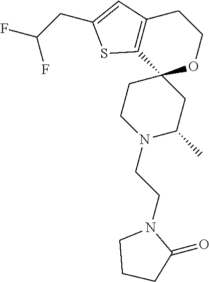 [SMILES: C[C@H]1C[C@@]2(CCN1CCN1CCCC1=O)OCCc1cc(CC(F)F)sc12] | 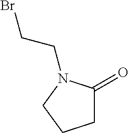 | LCMS m/z 398.66 [M + H]⁺.¹ |
| 624 | 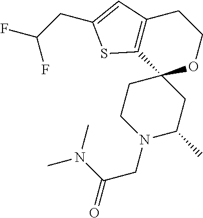 [SMILES: C[C@H]1C[C@@]2(CCN1CC(=O)N(C)C)OCCc1cc(CC(F)F)sc12] | 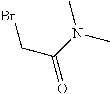 | LCMS m/z 373.17 [M + H]⁺.¹ |

Footnotes:
¹Reaction was stirred at 90° C. for 18 hours.

### Compound 625

### (2′S, 7R)-2-chloro-2′-methyl-1′-prop-2-ynyl-spiro[4,5-dihydrothieno[2,3-c]pyran-7,4′-piperidine]

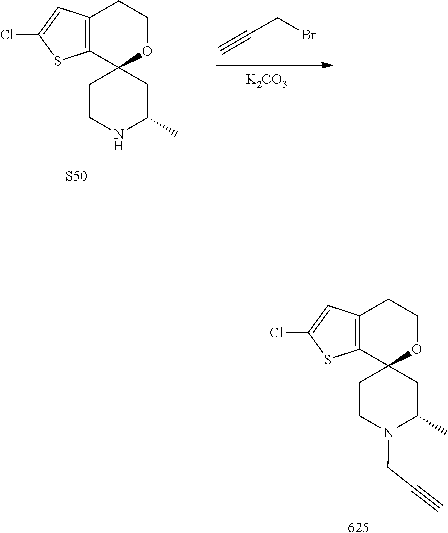

### Preparation of (2′S, 7R)-2-chloro-2′-methyl-1′-prop-2-ynyl-spiro[4,5-dihydrothieno[2,3-c]pyran-7,4′-piperidine](625)

A mixture of (2′S,7R)-2-chloro-2′-methyl-spiro[4,5-dihydrothieno[2,3-c]pyran-7,4′-piperidine](Trifluoroacetate salt) S50 (500 mg, 1.345 mmol) and propargyl bromide (267 mg, 1.796 mmol), and potassium carbonate (250 mg, 1.809 mmol) in acetonitrile (10 mL) was heated to 70° C. for 2 minutes. The mixture was cooled to room temperature, filtered, and concentrated. The crude material was diluted in dichloromethane (10 mL) and 2 M NaOH. The layers were mixed, passed over a phase separator, and concentrated. Purification by silica gel chromatography (1-10% MeOH in dichloromethane) afforded (2′S,7R)-2-chloro-2′-methyl-1′-prop-2-ynyl-spiro[4,5-dihydrothieno[2,3-c]pyran-7,4′-piperidine]625 (228 mg, 57%). ¹H NMR (400 MHz, Chloroform-d) δ 6.54 (s, 1H), 3.96-3.82 (m, 2H), 3.61 (dd, J=17.4, 2.4 Hz, 1H), 3.38 (dd, J=17.4, 2.4 Hz, 1H), 2.87 (ddd, J=12.8, 11.5, 2.7 Hz, 1H), 2.78 (dqd, J=12.5, 6.3, 2.6 Hz, 1H), 2.63 (ddd, J=11.4, 4.7, 2.4 Hz, 1H), 2.60-2.49 (m, 2H), 2.22 (t, J=2.4 Hz, 1H), 2.10-2.01 (m, 1H), 1.98 (dt, J=13.8, 2.8 Hz, 1H), 1.84 (ddd, J=13.9, 12.8, 4.6 Hz, 1H), 1.55 (dd, J=13.8, 11.5 Hz, 1H), 1.05 (d, J=6.3 Hz, 3H). LCMS m/z 296.36 [M+H]⁺.

### Compound 626

### 1-(4-(((2R,4S)-2′-chloro-2-methyl-4′,5′-dihydrospiro[piperidine-4,7′-thieno[2,3-c]pyran]-1-yl)methyl)-1H-1,2,3-triazol-1-yl)-3-methylbutane-2,3-diol

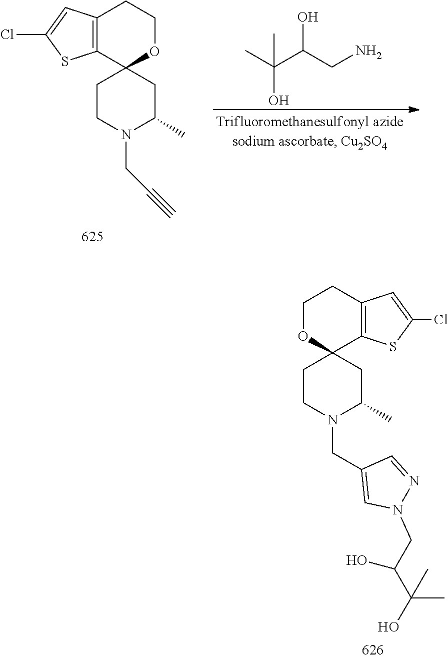

### Preparation of 1-(4-(((2R,4S)-2′-chloro-2-methyl-4′,5′-dihydrospiro[piperidine-4,7′-thieno[2,3-c]pyran]-1-yl)methyl)-1H-1,2,3-triazol-1-yl)-3-methylbutane-2,3-diol (626)

To a mixture of 1-amino-3-methyl-butane-2,3-diol (40 mg, 0.3357 mmol) in MeOH (3 mL) was added CuSO₄ (0.25 mg, 0.001566 mmol) in water (0.125 mL) followed by sodium bicarbonate (45 mg) in water (0.375 mL) and a solution of triflic azide (1.25 mL of 0.452 M, 0.5650 mmol) in dichloromethane and the solution was stirred for 1 hour. To the reaction was added (2′S,7R)-2-chloro-2′-methyl-1′-prop-2-ynyl-spiro[4,5-dihydrothieno[2,3-c]pyran-7,4′-piperidine]625 (50 mg, 0.1671 mmol) in methanol (0.5 mL), sodium ascorbate (35 mg, 0.1987 mmol) in water (0.25 mL) and 1-(1-benzyltriazol-4-yl)-N,N-bis[(1-benzyltriazol-4-yl)methyl]methanamine (2 mg, 0.003769 mmol) in methanol (0.2 mL), and the mixture was heated to 60° C. and stirred for 17 hours. The reactions were concentrated and purification by reverse phase HPLC (Method: C18 Waters Sunfire column (30×150 mm, 5 micron). Gradient: MeCN in H₂O with 0.1% trifluoroacetic acid) to afford 1-(4-(((2R,4S)-2′-chloro-2-methyl-4′,5′-dihydrospiro[piperidine-4,7′-thieno[2,3-c]pyran]-1-yl)methyl)-1H-1,2,3-triazol-1-yl)-3-methylbutane-2,3-diol (626) (51 mg, 69%). ¹H NMR (400 MHz, Chloroform-d) δ 7.80 (s, 1H), 6.59 (s, 1H), 4.73-4.60 (m, 1H), 4.34 (td, J=13.8, 13.3, 9.4 Hz, 1H), 3.95-3.78 (m, 4H), 2.75 (d, J=78.1 Hz, 4H), 2.65-2.54 (m, 2H), 2.22 (d, J=10.7 Hz, 1H), 2.04 (d, J=7.0 Hz, 3H), 1.79 (s, 2H), 1.47-1.27 (m, 9H). LCMS m/z 441.11 [M+H]⁺.

### Compounds 627-629

Compounds 627-629 (Table 28) were prepared from intermediate S50 and the appropriate amine reagent using the method for compound 626. Amines were obtained from commercial sources. Any modifications to methods are noted in Table 28 and accompanying footnotes.

TABLE 28
Structure and physicochemical data for compounds 627-629

| Compd | Structure | Amine | ¹H NMR; LCMS m/z [M + H]⁺ |
|---|---|---|---|
| 627 | 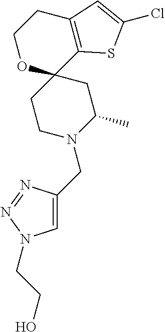 [SMILES: C[C@H]1C[C@@]2(CCN1Cc1cn(CCO)nn1)OCCc1cc(Cl)sc12] | 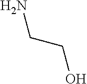 | ¹H NMR (400 MHz, Chloroform- d) δ 7.77 (s, 1H), 6.59 (s, 1H), 4.56-4.48 (m, 2H), 4.20 (d, J = 14.1 Hz, 1H), 4.14-4.07 (m, 2H), 3.95-3.81 (m, 3H), 2.80 (d, J = 60.8 Hz, 4H), 2.67-2.53 (m, 2H), 2.05 (d, J = 12.9 Hz, 2H), 1.96 (s, 1H), 1.34 (d, J = 6.0 Hz, 3H). LCMS m/z 382.99 [M + H]⁺. |
| 628 | 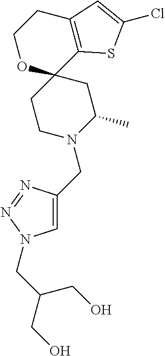 [SMILES: C[C@H]1C[C@@]2(CCN1Cc1cn(CC(CO)CO)nn1)OCCc1cc(Cl)sc12] | 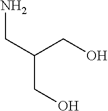 | ¹H NMR (400 MHz, Chloroform- d) δ 7.69 (s, 1H), 6.58 (s, 1H), 4.64 (d, J = 7.0 Hz, 2H), 4.14 (d, J = 14.6 Hz, 1H), 3.93-3.81 (m, 3H), 3.81-3.61 (m, 4H), 2.80 (d, J = 11.7 Hz, 1H), 2.67 (d, J = 9.7 Hz, 2H), 2.63-2.50 (m, 2H), 2.32 (tp, J = 7.0, 4.8 Hz, 1H), 2.04 (ddt, J = 11.2, 5.8, 2.8 Hz, 2H), 1.92 (d, J = 14.7 Hz, 1H), 1.74 (s, 1H), 1.30 (d, J = 6.2 Hz, 3H). LCMS m/z 427.07 [M + H]⁺. |
| 629 | 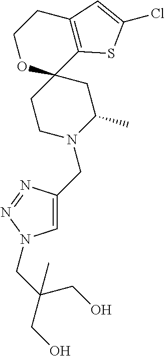 [SMILES: C[C@H]1C[C@@]2(CCN1Cc1cn(CC(C)(CO)CO)nn1)OCCc1cc(Cl)sc12] | 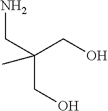 | ¹H NMR (400 MHZ, Chloroform- d) δ 7.76-7.56 (m, 1H), 6.58 (d, J = 1.4 Hz, 1H), 4.63-4.44 (m, 2H), 4.12 (d, J = 14.7 Hz, 1H), 3.87 (td, J = 14.1, 12.2, 8.4 Hz, 3H), 3.61 (dd, J = 11.3, 7.7 Hz, 2H), 3.43 (t, J = 10.0 Hz, 2H), 3.07 (s, 1H), 2.84-2.49 (m, 5H), 2.03 (t, J = 11.1 Hz, 2H), 1.91 (d, J = 13.9 Hz, 3H), 1.29 (d, J = 6.2 Hz, 3H), 0.86 (d, J = 1.6 Hz, 3H). LCMS m/z 441.07 [M + H]⁺. |

### Compound 630 #MZ48

### (2R,4S)-2′-chloro-2-methyl-1-((1-methyl-1H-pyrazol-4-yl)methyl)-4′,5′-dihydrospiro[piperidine-4,7′-thieno[2,3-c]pyran]

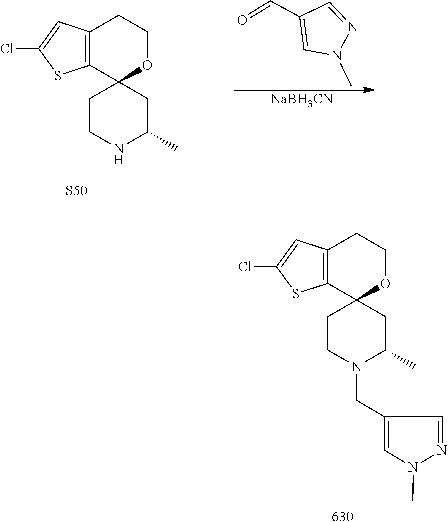

### Step 1: Synthesis of (2R,4S)-2′-chloro-2-methyl-1-((1-methyl-1H-pyrazol-4-yl)methyl)-4′,5′-dihydrospiro[piperidine-4,7′-thieno[2,3-c]pyran] (630)

1-methylpyrazole-4-carbaldehyde (12 mg, 0.109 mmol) and acetic acid (15 L, 0.266 mmol) were added to a solution of (2′S,7R)-2-chloro-2′-methyl-spiro[4,5-dihydrothieno[2,3-c]pyran-7,4′-piperidine]S50 (14 mg, 0.0290 mmol) in DMF (2 mL). NaBH₃CN (81 mg, 0.162 mmol) was added and the reaction mixture was stirred at 110° C. for 30 min. Purification by Prep-HPLC (Mobile phase A: −0.1% TFA (aq), Mobile phase B: -Acetonitrile) gave (2R,4S)-2′-chloro-2-methyl-1-((1-methyl-1H-pyrazol-4-yl)methyl)-4′,5′-dihydrospiro[piperidine-4,7′-thieno[2,3-c]pyran]630 (13 mg, 67%). ¹H NMR (300 MHz, Chloroform-d) δ 11.80 (s, 1H), 7.67 (s, 1H), 7.52 (s, 1H), 6.58 (s, 1H), 4.51 (d, J=14.2 Hz, 1H), 3.95 (s, 4H), 3.89-3.75 (m, 2H), 3.31 (d, J=13.0 Hz, 2H), 3.02 (d, J=9.6 Hz, 1H), 2.63-2.48 (m, 2H), 2.39-2.23 (m, 2H), 2.23-2.06 (m, 2H), 1.56 (d, J=6.5 Hz, 3H). LCMS m/z 352.12 [M+H]⁺.

### Compounds 631-693

Compounds 631-693 (Table 29) were prepared from intermediate S50 and the appropriate aldehyde reagent using the method for compound 630. Aldehydes were prepared by methods described above or obtained from commercial sources. Any modifications to methods are noted in Table 29 and accompanying footnotes.

TABLE 29
Structure and physicochemical data for compounds 631-693

| Compd | Structure | Aldehyde | ¹H NMR; LCMS m/z [M + H]⁺ |
|---|---|---|---|
| 631 | 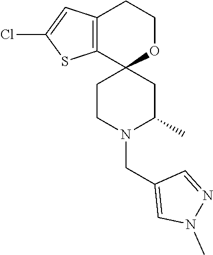 [SMILES: C[C@H]1C[C@@]2(CCN1Cc1cnn(C)c1)OCCc1cc(Cl)sc12] | 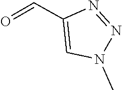 | |
| 632¹ | 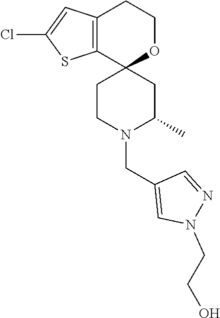 [SMILES: C[C@H]1C[C@@]2(CCN1Cc1cnn(CCO)c1)OCCc1cc(Cl)sc12] | 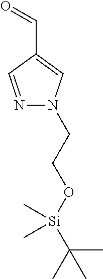 | ¹H NMR (400 MHz, Methanol- d4) δ 8.03 (d, J = 0.7 Hz, 1H), 7.81 (d, J = 0.8 Hz, 1H), 6.73 (s, 1H), 4.59 (d, J = 14.1 Hz, 1H), 4.31 (dd, J = 5.7, 4.8 Hz, 2H), 4.23 (d, J = 14.1 Hz, 1H), 3.98- 3.85 (m, 4H), 3.56 (ddd, J = 12.2, 6.4, 3.0 Hz, 1H), 3.41- 3.34 (m, 1H), 3.26 (dd, J = 12.8, 3.1 Hz, 1H), 2.61 (td, J = 5.3, 1.4 Hz, 2H), 2.34 (dt, J = 14.9, 3.0 Hz, 1H), 2.27 (dd, J = 15.0, 2.9 Hz, 1H), 2.12-1.99 (m, 3H), 1.56 (d, J = 6.4 Hz, 3H). LCMS m/z 382.0 [M + H]⁺. |
| 633 | 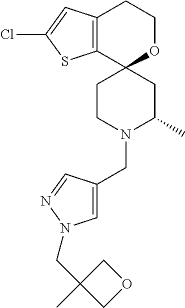 [SMILES: C[C@H]1C[C@@]2(CCN1Cc1cnn(CC3(C)COC3)c1)OCCc1cc(Cl)sc12] | 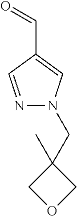 | ¹H NMR (400 MHZ, Chloroform-d) δ 7.41 (d, J = 0.7 Hz, 1H), 7.29 (s, 1H), 6.54 (s, 1H), 4.69 (dd, J = 6.2, 1.9 Hz, 2H), 4.38 (d, J = 6.1 Hz, 2H), 4.29 (s, 2H), 3.88-3.77 (m, 3H), 3.50 (d, J = 14.1 Hz, 1H), 2.67 (ddd, J = 11.6, 4.6, 2.5 Hz, 1H), 2.61-2.49 (m, 3H), 2.45 (td, J = 12.4, 11.7, 2.7 Hz, 1H), 1.98 (ddt, J = 15.2, 13.1, 2.9 Hz, 2H), 1.79 (td, J = 13.3, 4.5 Hz, 1H), 1.62 (dd, J = 13.9, 11.4 Hz, 1H), 1.22 (s, 3H), 1.18 (d, J = 6.2 Hz, 3H). LCMS m/z 422.13 [M + H]⁺. |
| 634 | 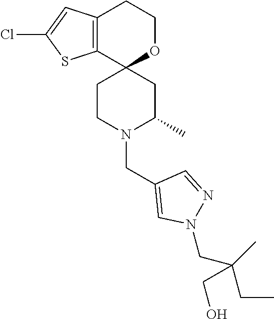 [SMILES: CCC(C)(CO)Cn1cc(CN2CC[C@]3(C[C@@H]2C)OCCc2cc(Cl)sc23)cn1] | 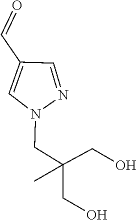 | ¹H NMR (400 MHZ, Chloroform-d) δ 7.45 (dd, J = 12.4, 0.7 Hz, 2H), 6.58 (d, J = 17.0 Hz, 1H), 4.32-4.19 (m, 2H), 3.93 (td, J = 11.5, 10.7, 5.4 Hz, 1H), 3.88-3.78 (m, 2H), 3.60-3.45 (m, 4H), 3.40-3.29 (m, 2H), 2.71 (d, J = 28.2 Hz, 2H), 2.63-2.47 (m, 3H), 2.10- 1.68 (m, 5H), 1.26 (d, J = 6.2 Hz, 3H), 0.84 (d, J = 10.1 Hz, 3H). LCMS m/z 440.12 [M + H]⁺. |
| 635 | 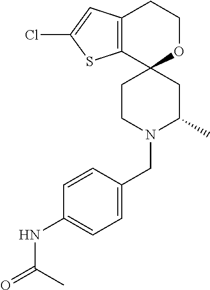 [SMILES: CC(=O)Nc1ccc(CN2CC[C@]3(C[C@@H]2C)OCCc2cc(Cl)sc23)cc1] | 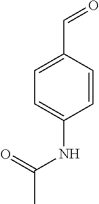 | ¹H NMR (400 MHZ, DMSO-d6) δ 10.14 (s, 1H), 9.56 (s, 1H), 7.67 (d, J = 8.2 Hz, 2H), 7.44 (d, J = 8.2 Hz, 2H), 6.92 (s, 1H), 4.64 (d, J = 12.9 Hz, 1H), 4.10 (dd, J = 13.0, 7.4 Hz, 1H), 3.88 (ddt, J = 19.1, 12.0, 5.7 Hz, 2H), 3.04 (dd, J = 28.7, 10.3 Hz, 2H), 2.57 (s, 2H), 2.29 (d, J = 14.7 Hz, 1H), 2.16 (d, J = 14.9 Hz, 1H), 2.06 (s, 3H), 2.00-1.85 (m, 2H), 1.48 (d, J = 6.5 Hz, 3H). LCMS m/z 405.35 [M + H]⁺. |
| 636 | 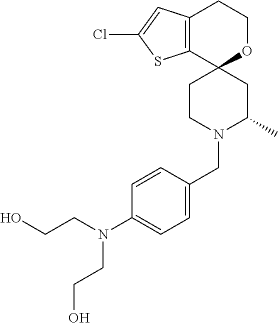 [SMILES: C[C@H]1C[C@@]2(CCN1Cc1ccc(N(CCO)CCO)cc1)OCCc1cc(Cl)sc12] | 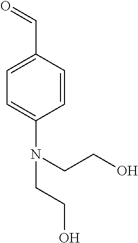 [SMILES: O=Cc1ccc(N(CCO)CCO)cc1] | ¹H NMR (400 MHZ, DMSO-d6) δ 9.29 (s, 1H), 7.29-7.21 (m, 2H), 6.92 (s, 1H), 6.73 (d, J = 8.5 Hz, 2H), 4.78 (s, 1H), 4.53 (d, J = 12.8 Hz, 1H), 3.98 (dd, J = 13.1, 7.3 Hz, 1H), 3.87 (dq, J = 18.2, 6.2 Hz, 2H), 3.54 (t, J = 6.2 Hz, 4H), 3.46-3.43 (m, 6H), 3.02 (d, J = 15.8 Hz, 2H), 2.57 (s, 2H), 2.28 (d, J = 14.9 Hz, 1H), 2.17 (d, J = 14.8 Hz, 1H), 1.89 (dt, J = 22.5, 11.6 Hz, 2H), 1.46 (d, J = 6.3 Hz, 3H). LCMS m/z 451.36 [M + H]⁺. |
| 637 | 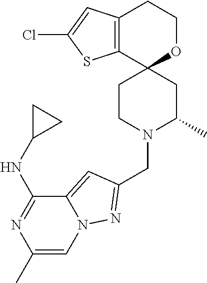 [SMILES: Cc1cn2nc(CN3CC[C@]4(C[C@@H]3C)OCCc3cc(Cl)sc34)cc2c(NC2CC2)n1] | 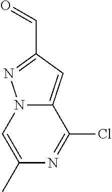 | ¹H NMR (400 MHZ, Methanol- d4) δ 7.65-7.61 (m, 1H), 6.82 (d, J = 0.9 Hz, 1H), 6.65 (s, 1H), 4.10 (d, J = 14.2 Hz, 1H), 3.93 (d, J = 14.2 Hz, 1H), 3.89-3.76 (m, 2H), 2.91 (tt, J = 7.2, 3.8 Hz, 1H), 2.86-2.68 (m, 3H), 2.54 (td, J = 5.5, 2.6 Hz, 2H), 2.33 (d, J = 1.0 Hz, 3H), 2.09-1.98 (m, 2H), 1.89-1.77 (m, 1H), 1.64 (dd, J = 14.1, 11.6 Hz, 1H), 1.30 (d, J = 6.3 Hz, 3H), 0.90-0.79 (m, 2H), 0.68-0.59 (m, 2H). LCMS m/z 458.18 [M + H]⁺. |
| 638 | 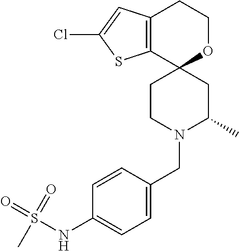 [SMILES: C[C@H]1C[C@@]2(CCN1Cc1ccc(NS(C)(=O)=O)cc1)OCCc1cc(Cl)sc12] | 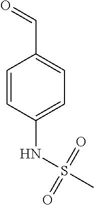 | ¹H NMR (400 MHZ, DMSO-d6) δ 10.05 (s, 1H), 7.48 (d, J = 8.1 Hz, 2H), 7.27 (d, J = 8.0 Hz, 2H), 6.92 (s, 1H), 4.67 (d, J = 12.8 Hz, 1H), 4.10 (dd, J = 13.0, 7.6 Hz, 1H), 3.87 (dp, J = 17.4, 5.9 Hz, 2H), 3.51 (s, 1H), 3.11 (s, 1H), 3.06 (s, 3H), 3.03-2.97 (m, 1H), 2.58 (s, 2H), 2.30 (d, J = 15.1 Hz, 1H), 2.16 (d, J = 15.3 Hz, 1H), 1.93 (dd, J = 23.3, 10.9 Hz, 2H), 1.48 (d, J = 6.3 Hz, 3H). LCMS m/z 441.29 [M + H]⁺. |
| 639 | 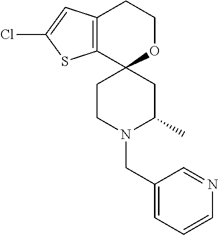 [SMILES: C[C@H]1C[C@@]2(CCN1Cc1cccnc1)OCCc1cc(Cl)sc12] | 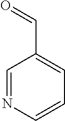 | ¹H NMR (400 MHz, DMSO-d6) δ 9.89 (s, 1H), 8.73 (d, J = 2.3 Hz, 1H), 8.68 (dd, J = 4.9, 1.6 Hz, 1H), 7.99 (d, J = 7.9 Hz, 1H), 7.55 (dd, J = 7.9, 4.8 Hz, 1H), 6.93 (s, 1H), 4.80 (d, J = 13.2 Hz, 1H), 4.22 (d, J = 13.1 Hz, 1H), 3.88 (dp, J = 22.6, 6.0, 5.6 Hz, 2H), 3.62 (dd, J = 6.0, 2.2 Hz, 1H), 3.16 (s, 1H), 3.01 (d, J = 8.8 Hz, 1H), 2.66-2.56 (m, 2H), 2.40-2.26 (m, 1H), 2.16 (d, J = 15.6 Hz, 1H), 2.02- 1.82 (m, 2H), 1.50 (d, J = 6.3 Hz, |
| | | | 3H). LCMS m/z 349.31 [M + H]⁺. |
| 640 | 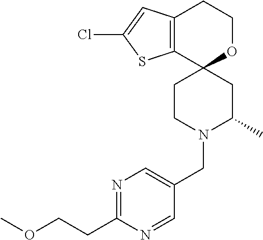 | 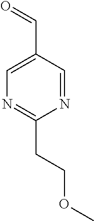 | ¹H NMR (400 MHZ, DMSO-d6) δ 8.87 (s, 2H), 6.93 (s, 1H), 4.79 (d, J = 13.3 Hz, 1H), 4.19 (s, 1H), 3.89 (dd, J = 11.6, 6.0 Hz, 3H), 3.83 (t, J = 6.5 Hz, 2H), 3.68-3.60 (m, 1H), 3.23 (s, 3H), 3.15 (t, J = 6.5 Hz, 2H), 2.59 (s, 2H), 2.32 (d, J = 13.2 Hz, 1H), 2.25-1.99 (m, 2H), 1.91 (d, J = 16.5 Hz, 2H), 1.48 (d, J = 6.1 Hz, 3H). LCMS m/z 408.33 [M + H]⁺. |
| 641 | 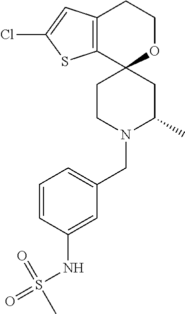 [SMILES: C[C@H]1C[C@@]2(CCN1Cc1cccc(NS(C)(=O)=O)c1)OCCc1cc(Cl)sc12] | 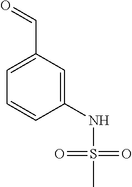 | LCMS m/z 441.33 [M + H]⁺. |
| 642 | 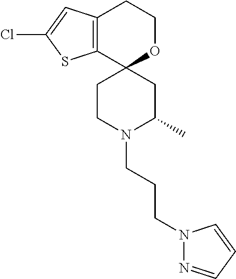 [SMILES: C[C@H]1C[C@@]2(CCN1CCCn1cccn1)OCCc1cc(Cl)sc12] | 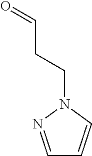 | LCMS m/z 366.3 [M + H]⁺. |
| 643 | 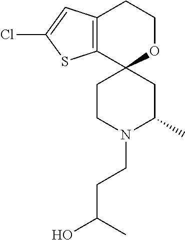 [SMILES: CC(O)CCN1CC[C@]2(C[C@@H]1C)OCCc1cc(Cl)sc12] | 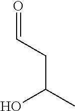 | ¹H NMR (400 MHZ, DMSO-d6) δ 9.37 (s, 1H), 8.32 (d, J = 12.7 Hz, 1H), 6.94 (s, 1H), 4.82 (s, 1H), 3.91 (d, J = 5.1 Hz, 2H), 3.69 (s, 1H), 3.16 (s, 2H), 2.59 (t, J = 5.5 Hz, 2H), 2.24 (t, J = 18.2 Hz, 2H), 2.07-1.83 (m, 2H), 1.71 (s, 1H), 1.28 (t, J = 5.7 Hz, 3H), 1.22 (t, J = 7.3 Hz, 2H), 1.18-1.13 (m, 1H), 1.14-1.08 (m, 2H). LCMS m/z 330.31 [M + H]⁺. |
| 644 | 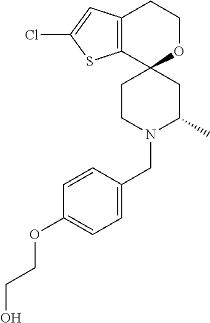 [SMILES: C[C@H]1C[C@@]2(CCN1Cc1ccc(OCCO)cc1)OCCc1cc(Cl)sc12] | 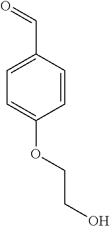 [SMILES: O=Cc1ccc(OCCO)cc1] | ¹H NMR (400 MHZ, DMSO-d6) δ 9.47 (s, 1H), 7.43 (d, J = 8.3 Hz, 2H), 7.04 (d, J = 8.3 Hz, 2H), 6.92 (s, 1H), 4.90 (s, 1H), 4.65 (d, J = 12.9 Hz, 1H), 4.09 (dd, J = 13.0, 7.5 Hz, 1H), 4.02 (t, J = 5.0 Hz, 2H), 3.87 (dp, J = 17.2, 5.9, 5.4 Hz, 2H), 3.72 (t, J = 4.8 Hz, 2H), 3.47 (s, 1H), 3.04 (dd, J = 30.4, 8.7 Hz, 2H), 2.58 (s, 2H), 2.29 (d, J = 15.1 Hz, 1H), 2.16 (d, J = 14.8 Hz, 1H), 1.91 (dd, J = 23.6, 11.4 Hz, 2H), 1.48 (d, J = 6.3 Hz, 3H). LCMS m/z 408.33 [M + H]⁺. |
| 645 | 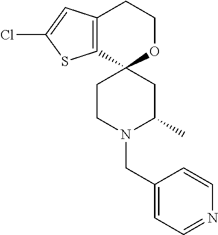 [SMILES: C[C@H]1C[C@@]2(CCN1Cc1ccncc1)OCCc1cc(Cl)sc12] | 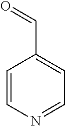 | ¹H NMR (400 MHZ, DMSO-d6) δ 10.09 (s, 1H), 8.71 (d, J = 5.0 Hz, 2H), 7.58 (d, J = 4.9 Hz, 2H), 6.92 (s, 1H), 4.78 (d, J = 13.4 Hz, 1H), 4.18 (d, J = 12.9 Hz, 1H), 3.88 (dp, J = 22.5, 5.8, 5.3 Hz, 2H), 3.14 (dd, J = 7.5, 4.1 Hz, 1H), 3.05-2.92 (m, 1H), 2.60-2.56 (m, 2H), 2.31 (d, J = 15.2 Hz, 1H), 2.14 (d, J = 15.0 Hz, 1H), 1.95 (q, J = 16.0, 15.4 Hz, 2H), 1.46 (d, J = 6.2 Hz, 3H). LCMS m/z 348.9 [M + H]⁺. |
| 646 | 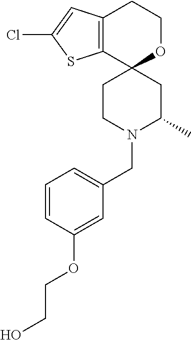 [SMILES: C[C@H]1C[C@@]2(CCN1Cc1cccc(OCCO)c1)OCCc1cc(Cl)sc12] | 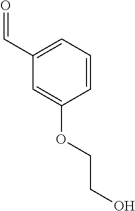 [SMILES: O=Cc1cccc(OCCO)c1] | ¹H NMR (400 MHz, DMSO-d6) δ 9.59 (s, 1H), 7.39 (t, J = 7.9 Hz, 1H), 7.17-6.98 (m, 3H), 6.92 (s, 1H), 4.91 (s, 1H), 4.71 (d, J = 12.8 Hz, 1H), 4.10 (dd, J = 13.0, 8.0 Hz, 1H), 4.02 (t, J = 5.0 Hz, 2H), 3.88 (dp, J = 22.7, 5.9, 5.4 Hz, 2H), 3.77-3.65 (m, 2H), 3.53 (s, 1H), 3.12 (q, J = 11.6, 11.2 Hz, 1H), 3.00-2.86 (m, 1H), 2.58 (s, 2H), 2.37-2.23 (m, 1H), 2.17 (d, J = 15.0 Hz, 1H), 1.93 (dt, J = 25.9, 12.7 Hz, 2H), 1.48 (d, J = 6.3 Hz, 3H). LCMS m/z 408.33 [M + H]⁺. |
| 647 | 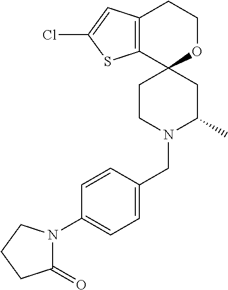 [SMILES: C[C@H]1C[C@@]2(CCN1Cc1ccc(N3CCCC3=O)cc1)OCCc1cc(Cl)sc12] | 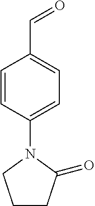 [SMILES: O=Cc1ccc(N2CCCC2=O)cc1] | ¹H NMR (400 MHZ, DMSO-d6) δ 9.58 (s, 1H), 7.78 (d, J = 8.3 Hz, 2H), 7.52 (d, J = 8.4 Hz, 2H), 6.92 (s, 1H), 4.70 (d, J = 12.9 Hz, 1H), 4.14 (dd, J = 13.0, 7.4 Hz, 1H), 3.87 (dt, J = 14.9, 6.5 Hz, 4H), 3.09 (t, J = 11.7 Hz, 1H), 3.00 (s, 1H), 2.93 (dd, J = 15.9, 9.7 Hz, 1H), 2.58 (s, 2H), 2.52 (s, 1H), 2.30 (d, J = 15.2 Hz, 1H), 2.16 (d, J = 15.4 Hz, 1H), 2.07 (p, J = 7.6 Hz, 2H), 1.91 (dt, J = 24.7, 13.8 Hz, 2H), 1.48 (d, J = 6.2 Hz, 3H). LCMS m/z 431.35 [M + H]⁺. |
| 648 | 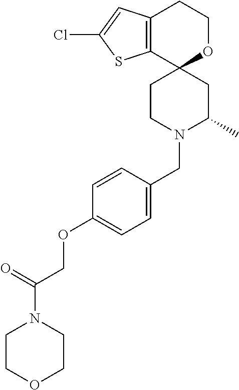 [SMILES: C[C@H]1C[C@@]2(CCN1Cc1ccc(OCC(=O)N3CCOCC3)cc1)OCCc1cc(Cl)sc12] | 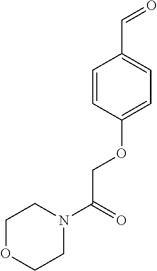 [SMILES: O=Cc1ccc(OCC(=O)N2CCOCC2)cc1] | ¹H NMR (400 MHZ, DMSO-d6) δ 9.56 (s, 1H), 7.43 (d, J = 8.3 Hz, 2H), 7.02 (s, 2H), 6.92 (s, 1H), 4.89 (s, 2H), 4.80-4.56 (m, 1H), 4.09 (dd, J = 13.0, 7.5 Hz, 1H), 3.88 (dq, J = 18.3, 6.2 Hz, 2H), 3.61 (s, 3H), 3.57 (s, 4H), 3.18-2.95 (m, 3H), 2.58 (s, 2H), 2.39-2.23 (m, 1H), 2.17 (d, J = 14.7 Hz, 1H), 1.92 (q, J = 16.3, 15.3 Hz, 2H), 1.48 (d, J = 6.3 Hz, 3H). LCMS m/z 491.41 [M + H]⁺. |
| 649 | 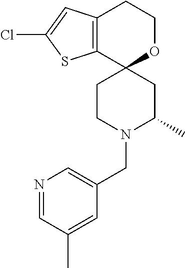 [SMILES: Cc1cncc(CN2CC[C@]3(C[C@@H]2C)OCCc2cc(Cl)sc23)c1] | 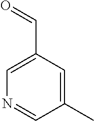 | ¹H NMR (400 MHz, DMSO-d6) δ 8.54 (s, 2H), 7.83 (s, 1H), 6.93 (s, 1H), 4.77 (d, J = 13.0 Hz, 1H), 4.16 (d, J = 13.1 Hz, 1H), 3.94-3.80 (m, 2H), 3.55 (s, 1H), 3.14 (s, 1H), 3.01 (d, J = 9.6 Hz, 1H), 2.58 (s, 2H), 2.36 (s, 3H), 2.15 (d, J = 15.1 Hz, 1H), 1.92 (dt, J = 23.3, 13.2 Hz, 2H), 1.49 (d, J = 6.3 Hz, 3H). LCMS m/z 363.31 [M + H]⁺. |
| 650 | 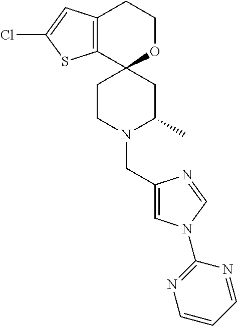 [SMILES: C[C@H]1C[C@@]2(CCN1Cc1cn(-c3ncccn3)cn1)OCCc1cc(Cl)sc12] | 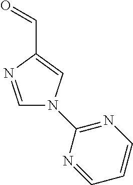 [SMILES: O=Cc1cn(-c2ncccn2)cn1] | ¹H NMR (400 MHZ, DMSO-d6) δ 9.88 (s, 1H), 8.93 (d, J = 4.9 Hz, 2H), 8.76 (d, J = 1.3 Hz, 1H), 8.26 (s, 1H), 7.57 (t, J = 4.9 Hz, 1H), 6.92 (s, 1H), 4.51 (d, J = 14.0 Hz, 1H), 4.40 (d, J = 14.1 Hz, 1H), 3.84 (p, J = 6.0 Hz, 2H), 3.39 (d, J = 13.5 Hz, 1H), 3.27 (s, 1H), 2.56 (s, 2H), 2.24 (dd, J = 25.6, 14.7 Hz, 2H), 1.97 (q, J = 14.5, 13.8 Hz, 2H), 1.48 (d, J = 6.3 Hz, 3H). LCMS m/z 416.28 [M + H]⁺. |
| 651 | 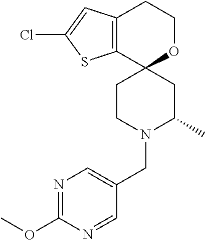 | 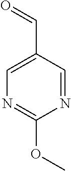 | ¹H NMR (400 MHZ, DMSO-d6) δ 8.74 (s, 2H), 6.93 (d, J = 2.0 Hz, 1H), 4.74 (d, J = 13.3 Hz, 1H), 4.15 (d, J = 11.5 Hz, 1H), 3.96 (s, 3H), 3.90 (dt, J = 11.4, 5.7 Hz, 3H), 3.13 (s, 2H), 2.59 (s, 2H), 2.32 (d, J = 14.1 Hz, 1H), 2.16 (d, J = 14.9 Hz, 1H), 1.99-1.81 (m, 2H), 1.48 (d, J = 6.3 Hz, 3H). LCMS m/z 380.33 [M + H]⁺. |
| 652 | 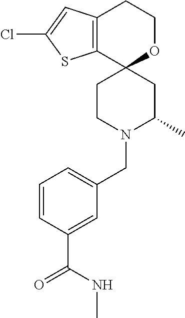 [SMILES: CNC(=O)c1cccc(CN2CC[C@]3(C[C@@H]2C)OCCc2cc(Cl)sc23)c1] | 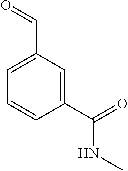 | LCMS m/z 405.35 [M + H]⁺. |
| 653 | 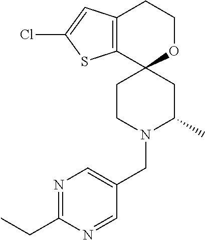 [SMILES: CCc1ncc(CN2CC[C@]3(C[C@@H]2C)OCCc2cc(Cl)sc23)cn1] | 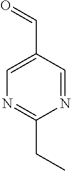 | ¹H NMR (400 MHZ, DMSO-d6) δ 8.87 (s, 2H), 6.93 (s, 1H), 4.79 (d, J = 13.1 Hz, 1H), 4.19 (s, 1H), 3.99-3.83 (m, 3H), 3.13 (d, J = 33.9 Hz, 2H), 2.93 (t, J = 7.6 Hz, 2H), 2.59 (s, 2H), 2.32 (d, J = 14.8 Hz, 1H), 2.15 (d, J = 15.1 Hz, 1H), 1.91 (q, J = 15.7, 15.3 Hz, 2H), 1.49 (d, J = 6.3 Hz, 3H), 1.30 (t, J = 7.6 Hz, 3H). LCMS m/z 378.35 [M + H]⁺. |
| 654 | 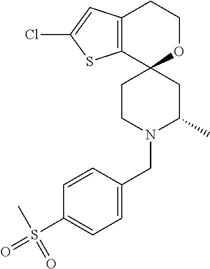 [SMILES: C[C@H]1C[C@@]2(CCN1Cc1ccc(S(C)(=O)=O)cc1)OCCc1cc(Cl)sc12] | 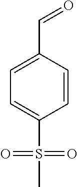 | ¹H NMR (400 MHz, DMSO-d6) δ 9.85 (s, 1H), 8.05 (d, J = 8.0 Hz, 2H), 7.81 (d, J = 8.0 Hz, 2H), 6.93 (s, 1H), 4.88 (d, J = 12.9 Hz, 1H), 4.28 (s, 1H), 3.89 (ddt, J = 18.8, 13.0, 6.6 Hz, 2H), 3.58 (s, 1H), 3.27 (s, 3H), 3.03- 2.77 (m, 2H), 2.58 (s, 2H), 2.39- 2.23 (m, 1H), 2.15 (d, J = 14.8 Hz, 1H), 1.93 (dt, J = 28.2, 13.9 Hz, 2H), 1.49 (d, J = 6.2 Hz, 3H). LCMS m/z 426.26 [M + H]⁺. |
| 655 | 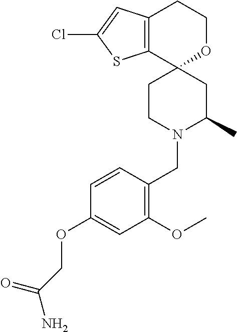 [SMILES: COc1cc(OCC(N)=O)ccc1CN1CC[C@@]2(C[C@H]1C)OCCc1cc(Cl)sc12] | 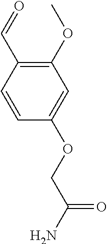 | ¹H NMR (400 MHZ, DMSO-d6) δ 9.62 (s, 1H), 7.42 (s, 1H), 7.36 (s, 1H), 7.18 (d, J = 1.9 Hz, 1H), 7.06-7.00 (m, 1H), 6.96 (d, J = 8.2 Hz, 1H), 6.92 (s, 1H), 4.75- 4.61 (m, 1H), 4.47 (s, 2H), 4.04 (dd, J = 13.0, 8.0 Hz, 1H), 3.90 (dq, J = 13.4, 6.2 Hz, 2H), 3.83 (s, 3H), 3.10 (q, J = 11.3 Hz, 1H), 2.95 (dd, J = 13.9, 8.1 Hz, 1H), 2.58 (s, 2H), 2.30 (d, J = 15.1 Hz, 1H), 2.16 (d, J = 14.8 Hz, 1H), 1.93 (dt, J = 26.4, 12.6 Hz, 2H), 1.48 (d, J = 6.3 Hz, 3H). LCMS m/z 451.31 [M + H]⁺. |
| 656 | 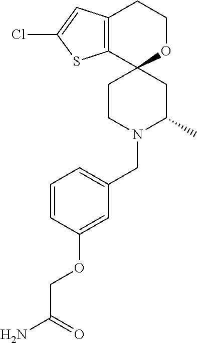 [SMILES: C[C@H]1C[C@@]2(CCN1Cc1cccc(OCC(N)=O)c1)OCCc1cc(Cl)sc12] | 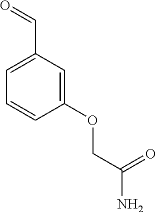 [SMILES: NC(=O)COc1cccc(C=O)c1] | ¹H NMR (400 MHZ, DMSO-d6) δ 9.58 (s, 1H), 7.56 (s, 1H), 7.47- 7.40 (m, 2H), 7.12 (d, J = 11.4 Hz, 2H), 7.06 (s, 1H), 7.02 (d, J = 12.4 Hz, 1H), 6.93 (s, 1H), 4.70 (d, J = 13.9 Hz, 1H), 4.48 (s, 2H), 4.11 (dd, J = 12.9, 7.8 Hz, 1H), 3.92-3.83 (m, 2H), 2.99-2.87 (m, 1H), 2.58 (s, 2H), 2.37-2.21 (m, 1H), 2.16 (d, J = 15.0 Hz, 1H), 1.92 (dt, J = 24.7, 12.8 Hz, 2H), 1.48 (d, J = 6.4 Hz, 3H). LCMS m/z 421.33 [M + H]⁺. |
| 657 | 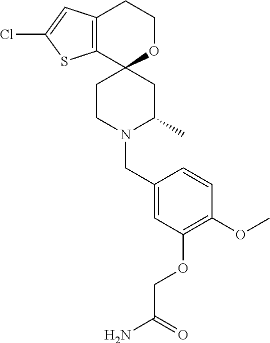 [SMILES: COc1ccc(CN2CC[C@]3(C[C@@H]2C)OCCc2cc(Cl)sc23)cc1OCC(N)=O] | 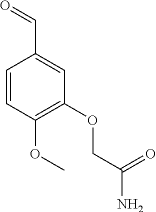 | ¹H NMR (400 MHZ, DMSO-d6) δ 7.46 (s, 1H), 7.29 (d, J = 18.9 Hz, 1H), 7.10 (s, 3H), 6.93 (s, 1H), 4.71-4.60 (m, 1H), 4.47 (s, 2H), 4.05 (dd, J = 12.8, 7.5 Hz, 1H), 3.89 (dq, J = 12.6, 6.2 Hz, 2H), 3.82 (s, 3H), 3.53-3.47 (m, 1H), 3.06 (t, J = 11.6 Hz, 1H), 2.93 (dd, J = 13.1, 7.0 Hz, 1H), 2.58 (s, 2H), 2.30 (d, J = 15.3 Hz, 1H), 2.15 (d, J = 15.0 Hz, 1H), 1.98-1.78 (m, 2H), 1.47 (d, J = 6.3 Hz, 3H). LCMS m/z 451.31 [M + H]⁺. |
| 658 | 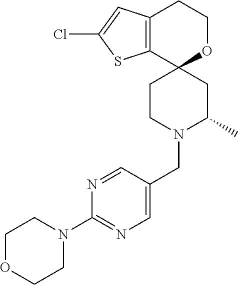 [SMILES: C[C@H]1C[C@@]2(CCN1Cc1cnc(N3CCOCC3)nc1)OCCc1cc(Cl)sc12] | 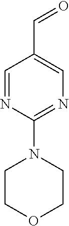 [SMILES: O=Cc1cnc(N2CCOCC2)nc1] | ¹H NMR (400 MHZ, DMSO-d6) δ 9.67 (s, 1H), 8.49 (s, 2H), 6.93 (s, 1H), 4.60 (d, J = 13.5 Hz, 1H), 4.04 (dd, J = 13.5, 7.0 Hz, 1H), 3.88 (dq, J = 11.9, 5.8 Hz, 3H), 3.74 (d, J = 4.9 Hz, 3H), 3.66 (t, J = 4.6 Hz, 4H), 3.10 (d, J = 20.3 Hz, 2H), 2.58 (t, J = 5.6 Hz, 2H), 2.31 (d, J = 15.2 Hz, 1H), 2.17 (d, J = 14.9 Hz, 1H), 1.90 (t, J = 13.2 Hz, 2H), 1.46 (d, J = 6.3 Hz, 3H). LCMS m/z 435.37 [M + H]⁺. |
| 659 | 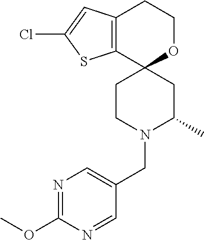 | 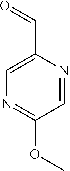 | ¹H NMR (400 MHZ, DMSO-d6) δ 8.46 (d, J = 1.4 Hz, 1H), 8.41 (s, 1H), 6.92 (s, 1H), 4.71 (d, J = 13.7 Hz, 1H), 4.39 (s, 1H), 3.96 (s, 3H), 3.86 (tq, J = 11.6, 5.8 Hz, 2H), 3.58 (s, 1H), 3.18 (s, 2H), 2.57 (t, J = 5.6 Hz, 2H), 2.27 (d, J = 14.8 Hz, 1H), 2.14 (d, J = 14.8 Hz, 1H), 2.06-1.86 (m, 2H), 1.45 (d, J = 6.3 Hz, 3H). LCMS m/z 380.29 [M + H]⁺. |
| 660 | 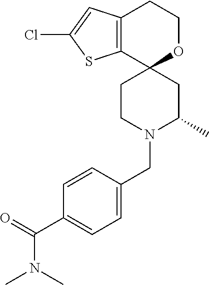 | 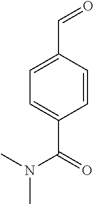 | ¹H NMR (400 MHZ, DMSO-d6) δ 7.59 (d, J = 7.9 Hz, 2H), 7.51 (d, J = 7.9 Hz, 2H), 6.92 (s, 1H), 4.78 (d, J = 12.9 Hz, 1H), 4.19 (dd, J = 13.0, 7.7 Hz, 1H), 3.88 (dp, J = 17.3, 6.0, 5.5 Hz, 3H), 3.56 (s, 1H), 3.00 (s, 4H), 2.90 (s, 3H), 2.58 (s, 2H), 2.46-2.24 (m, 1H), 2.16 (d, J = 15.4 Hz, 1H), 2.03-1.80 (m, 2H), 1.49 (d, J = 6.3 Hz, 3H). LCMS m/z 419.3 [M + H]⁺. |
| 661 | 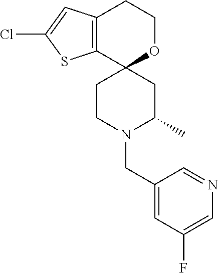 [SMILES: C[C@H]1C[C@@]2(CCN1Cc1cncc(F)c1)OCCc1cc(Cl)sc12] | 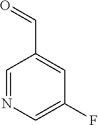 | 1H NMR (400 MHZ, DMSO-d6) δ 8.71 (s, 1H), 8.61 (s, 1H), 7.95 (d, J = 9.6 Hz, 1H), 6.93 (s, 1H), 4.85 (d, J = 13.1 Hz, 1H), 4.24 (s, 1H), 3.91 (ddt, J = 18.2, 11.9, 6.2 Hz, 3H), 3.19 (s, 1H), 3.02 (d, J = 17.4 Hz, 1H), 2.58 (d, J = 5.5 Hz, 2H), 2.37-2.24 (m, 1H), 2.15 (d, J = 15.4 Hz, 1H), 1.92 (q, J = 15.3, 14.0 Hz, 2H), 1.48 (d, J = 6.3 Hz, 3H). LCMS m/z 367.33 [M + H]⁺. |
| 662 | 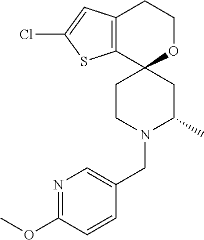 | 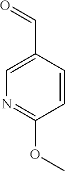 | ¹H NMR (400 MHZ, DMSO-d6) δ 8.31 (d, J = 2.3 Hz, 1H), 7.85 (dd, J = 8.5, 2.5 Hz, 1H), 6.94 (d, J = 11.8 Hz, 2H), 4.70 (d, J = 13.1 Hz, 1H), 4.13 (d, J = 12.8 Hz, 1H), 3.88 (s, 6H), 3.07 (d, J = 25.8 Hz, 2H), 2.57 (d, J = 5.9 Hz, 2H), 2.31 (d, J = 15.0 Hz, 1H), 2.16 (d, J = 15.2 Hz, 1H), 1.91 (q, J = 15.8, 14.9 Hz, 2H), 1.48 (d, J = 6.3 Hz, 3H). LCMS m/z 379.34 [M + H]⁺. |
| 663 | 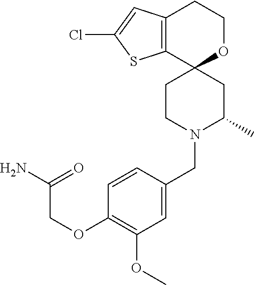 [SMILES: COc1cc(CN2CC[C@]3(C[C@@H]2C)OCCc2cc(Cl)sc23)ccc1OCC(N)=O] | 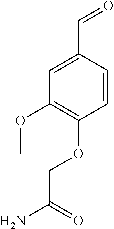 | ¹H NMR (400 MHZ, DMSO-d6) δ 7.42 (s, 1H), 7.36 (s, 1H), 7.18 (s, 1H), 7.06-7.00 (m, 1H), 6.96 (d, J = 8.3 Hz, 1H), 6.92 (s, 1H), 4.74-4.63 (m, 1H), 4.47 (s, 2H), 4.03 (dd, J = 12.9, 7.9 Hz, 1H), 4.00-3.84 (m, 3H), 3.83 (s, 3H), 2.96 (dd, J = 22.1, 8.7 Hz, 2H), 2.58 (s, 2H), 2.39-2.24 (m, 1H), 2.16 (d, J = 14.9 Hz, 1H), 1.93 (dt, J = 26.6, 13.1 Hz, 2H), 1.48 (d, J = 6.3 Hz, 3H). LCMS m/z 451.31 [M + H]⁺ |
| 664 | 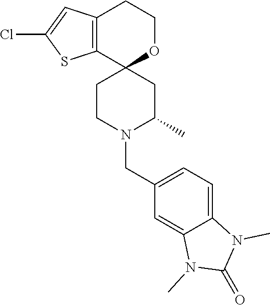 [SMILES: C[C@H]1C[C@@]2(CCN1Cc1ccc3c(c1)n(C)c(=O)n3C)OCCc1cc(Cl)sc12] | 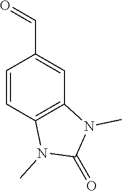 | LCMS m/z 432.35 [M + H]⁺. |
| 665 | 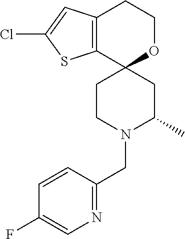 [SMILES: C[C@H]1C[C@@]2(CCN1Cc1ccc(F)cn1)OCCc1cc(Cl)sc12] | 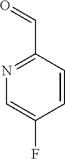 | ¹H NMR (400 MHZ, DMSO-d6) δ 8.74 (d, J = 3.0 Hz, 1H), 7.91 (t, J = 8.6 Hz, 1H), 7.72-7.60 (m, 1H), 6.92 (s, 1H), 4.78 (d, J = 14.0 Hz, 1H), 4.41 (s, 1H), 3.87 (dq, J = 11.9, 5.8 Hz, 2H), 3.60 (s, 1H), 3.18 (d, J = 29.0 Hz, 2H), 2.64-2.56 (m, 2H), 2.26 (d, J = 14.6 Hz, 1H), 2.07 (dt, J = 43.7, 15.7 Hz, 3H), 1.42 (d, J = 6.3 Hz, 3H). LCMS m/z 367.29 [M + H]⁺. |
| 666 | 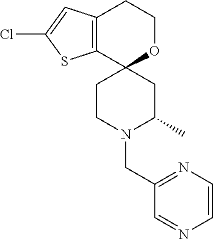 [SMILES: C[C@H]1C[C@@]2(CCN1Cc1cnccn1)OCCc1cc(Cl)sc12] | 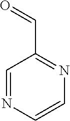 | ¹H NMR (400 MHZ, DMSO-d6) δ 8.83 (s, 1H), 8.79 (d, J = 11.4 Hz, 2H), 6.92 (s, 1H), 4.85 (s, 1H), 4.50 (s, 1H), 3.88 (dq, J = 12.0, 5.8 Hz, 2H), 3.64 (s, 1H), 3.23 (s, 2H), 2.58 (s, 2H), 2.28 (d, J = 15.1 Hz, 1H), 2.21-1.92 (m, 3H), 1.42 (d, J = 6.3 Hz, 3H). LCMS m/z 350.31 [M + H]⁺. |
| 667 | 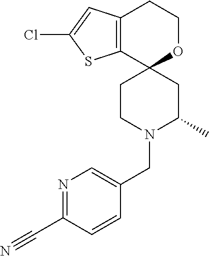 [SMILES: C[C@H]1C[C@@]2(CCN1Cc1ccc(C#N)nc1)OCCc1cc(Cl)sc12] | 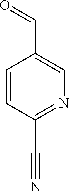 | ¹H NMR (400 MHZ, DMSO-d6) δ 8.89 (s, 1H), 8.21 (s, 2H), 6.96 (d, J = 28.7 Hz, 1H), 4.90 (d, J = 8.2 Hz, 1H), 4.29 (s, 1H), 3.89 (d, J = 11.8 Hz, 2H), 3.59 (s, 1H), 3.20 (s, 1H), 2.98-2.87 (m, 1H), 2.58 (s, 2H), 2.35 (d, J = 17.1 Hz, 1H), 2.12 (s, 1H), 1.88 (s, 2H), 1.48 (s, 3H). LCMS m/z 374.33 [M + H]⁺. |
| 668 | 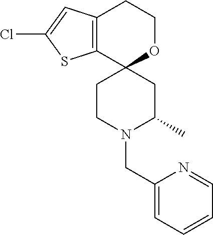 [SMILES: C[C@H]1C[C@@]2(CCN1Cc1ccccn1)OCCc1cc(Cl)sc12] | 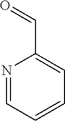 | ¹H NMR (400 MHZ, DMSO-d6) δ 8.72 (d, J = 4.8 Hz, 1H), 7.95 (td, J = 7.8, 1.8 Hz, 1H), 7.58 (d, J = 7.7 Hz, 1H), 7.50 (dd, J = 7.6, 4.9 Hz, 1H), 6.92 (s, 1H), 4.79 (d, J = 14.2 Hz, 1H), 4.41 (d, J = 14.3 Hz, 1H), 3.88 (ddq, J = 17.4, 11.6, 5.4 Hz, 2H), 3.64 (s, 1H), 3.25 (s, 1H), 3.19-3.09 (m, 1H), 2.60-2.56 (m, 2H), 2.25 (dt, J = 14.7, 3.0 Hz, 1H), 2.20-1.96 (m, 3H), 1.40 (d, J = 6.4 Hz, 3H). LCMS m/z 349.31 [M + H]⁺. |
| 669 | 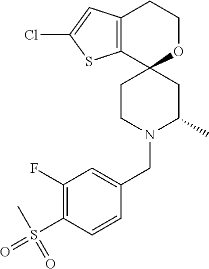 [SMILES: C[C@H]1C[C@@]2(CCN1Cc1ccc(S(C)(=O)=O)c(F)c1)OCCc1cc(Cl)sc12] | 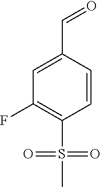 | ¹H NMR (400 MHZ, DMSO-d6) δ 7.98 (s, 1H), 7.76 (dd, J = 16.3, 8.8 Hz, 1H), 7.62 (s, 1H), 6.92 (s, 1H), 4.88 (s, 1H), 4.25 (s, 1H), 3.88 (dd, J = 15.0, 9.3 Hz, 2H), 3.37 (s, 3H), 3.32 (s, 1H), 3.00-2.85 (m, 2H), 2.59 (s, 2H), 2.35 (d, J = 16.5 Hz, 1H), 2.13 (s, 1H), 1.94 (s, 2H), 1.48 (s, 3H). LCMS m/z 444.27 [M + H]⁺. |
| 670 | 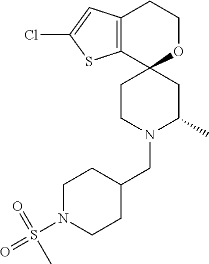 [SMILES: C[C@H]1C[C@@]2(CCN1CC1CCN(S(C)(=O)=O)CC1)OCCc1cc(Cl)sc12] | 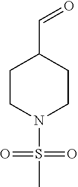 | ¹H NMR (400 MHZ, DMSO-d6) δ 6.93 (d, J = 4.4 Hz, 1H), 3.91 (q, J = 5.4 Hz, 2H), 3.69-3.57 (m, 2H), 3.56-3.49 (m, 1H), 3.29 (d, J = 11.4 Hz, 1H), 3.15 (dd, J = 12.1, 8.4 Hz, 1H), 2.98 (d, J = 13.9 Hz, 1H), 2.87 (s, 3H), 2.69 (d, J = 14.4 Hz, 2H), 2.60 (t, J = 5.4 Hz, 2H), 2.22 (dd, J = 25.4, 14.7 Hz, 2H), 2.15- 1.87 (m, 4H), 1.74 (d, J = 13.1 Hz, 1H), 1.32 (d, J = 6.2 Hz, 4H), 1.27 (s, 1H). LCMS m/z 433.3 [M + H]⁺. |
| 671 | 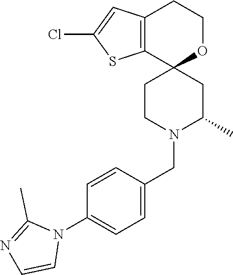 [SMILES: Cc1nccn1-c1ccc(CN2CC[C@]3(C[C@@H]2C)OCCc2cc(Cl)sc23)cc1] | 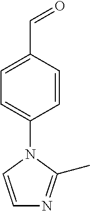 | LCMS m/z 428.33 [M + H]⁺. |
| 672 | 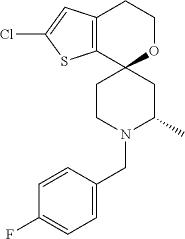 [SMILES: C[C@H]1C[C@@]2(CCN1Cc1ccc(F)cc1)OCCc1cc(Cl)sc12] | 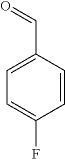 | ¹H NMR (400 MHZ, DMSO-d6) δ 7.59-7.50 (m, 2H), 7.34 (t, J = 8.6 Hz, 2H), 6.92 (s, 1H), 4.73 (d, J = 12.9 Hz, 1H), 4.32-3.98 (m, 1H), 3.88 (dtd, J = 17.4, 11.5, 5.4 Hz, 2H), 3.52 (s, 1H), 3.12 (d, J = 12.2 Hz, 1H), 3.03- 2.87 (m, 1H), 2.59 (d, J = 6.0 Hz, 2H), 2.31 (d, J = 15.2 Hz, 1H), 2.16 (d, J = 15.5 Hz, 1H), 1.91 (dt, J = 25.7, 13.7 Hz, 2H), 1.48 (d, J = 6.2 Hz, 3H). LCMS m/z 366.29 [M + H]⁺. |
| 673 | 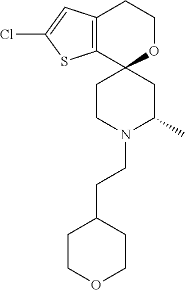 [SMILES: C[C@H]1C[C@@]2(CCN1CCC1CCOCC1)OCCc1cc(Cl)sc12] | 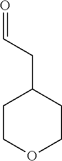 | ¹H NMR (400 MHZ, DMSO-d6) δ 6.94 (s, 1H), 3.90 (q, J = 5.4 Hz, 2H), 3.86-3.82 (m, 2H), 3.44 (d, J = 12.4 Hz, 1H), 3.27 (dd, J = 13.3, 10.5 Hz, 4H), 3.19 (d, J = 11.3 Hz, 1H), 3.15-3.03 (m, 1H), 2.59 (t, J = 5.5 Hz, 2H), 2.30-2.18 (m, 2H), 2.00 (t, J = 12.3 Hz, 1H), 1.94-1.81 (m, 1H), 1.58 (t, J = 13.9 Hz, 5H), 1.29 (d, J = 6.3 Hz, 3H), 1.26 - 1.19 (m, 2H). LCMS m/z 370.35 [M + H]⁺. |
| 674 | 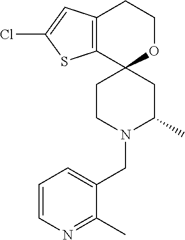 [SMILES: Cc1ncccc1CN1CC[C@]2(C[C@@H]1C)OCCc1cc(Cl)sc12] | 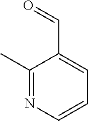 | ¹H NMR (400 MHZ, DMSO-d6) δ 8.58 (d, J = 5.0 Hz, 1H), 7.99 (s, 1H), 7.44 (s, 1H), 6.93 (s, 1H), 4.83 (d, J = 13.3 Hz, 1H), 4.24 (s, 1H), 3.93 (dd, J = 14.0, 5.9 Hz, 2H), 3.74 (s, 1H), 3.01 (d, J = 9.5 Hz, 1H), 2.93 (q, J = 6.6 Hz, 1H), 2.65 (s, 3H), 2.61- 2.57 (m, 2H), 2.38-2.27 (m, 1H), 2.13 (d, J = 14.9 Hz, 1H), 1.95 (dt, J = 29.4, 13.1 Hz, 2H), 1.51 (d, J = 6.2 Hz, 3H). LCMS m/z 363.31 [M + H]⁺. |
| 675 | 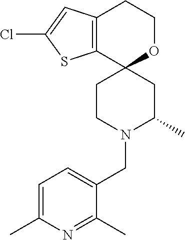 | 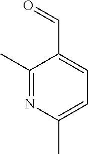 | ¹H NMR (400 MHZ, DMSO-d6) δ 7.98 (s, 1H), 7.39 (s, 1H), 6.93 (s, 1H), 4.79 (s, 1H), 4.23 (s, 1H), 3.92 (dq, J = 17.9, 6.0 Hz, 5H), 3.73 (s, 1H), 3.23 (s, 1H), 3.00 (s, 1H), 2.64 (s, 3H), 2.60 (s, 2H), 2.30 (s, 1H), 2.12 (d, J = 15.0 Hz, 1H), 2.07-1.80 (m, 2H), 1.50 (s, 3H). LCMS m/z 377.35 [M + H]⁺. |
| 676 | 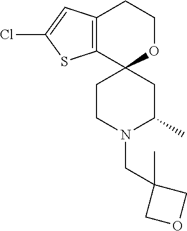 [SMILES: C[C@H]1C[C@@]2(CCN1CC1(C)COC1)OCCc1cc(Cl)sc12] | 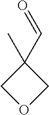 | ¹H NMR (400 MHZ, DMSO-d6) δ 6.93 (d, J = 1.8 Hz, 1H), 4.66 (d, J = 6.0 Hz, 1H), 4.39 (d, J = 6.2 Hz, 1H), 4.27 (d, J = 5.9 Hz, 1H), 4.19 (d, J = 6.2 Hz, 1H), 3.91 (d, J = 7.0 Hz, 3H), 3.22 (d, J = 10.8 Hz, 1H), 2.88 (d, J = 13.7 Hz, 1H), 2.59 (s, 2H), 2.11 (ddd, J = 58.3, 31.1, 14.8 Hz, 5H), 1.49 (s, 3H), 1.31 (d, J = 6.4 Hz, 3H). LCMS m/z 342.32 [M + H]⁺. |
| 677 | 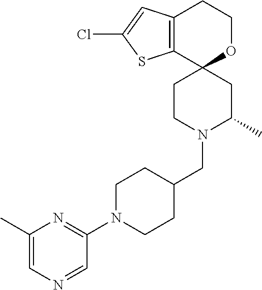 | 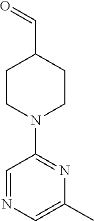 | LCMS m/z 447.38 [M + H]⁺. |
| 678 | 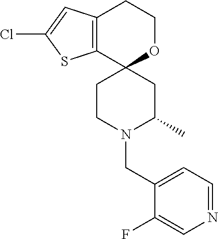 [SMILES: C[C@H]1C[C@@]2(CCN1Cc1ccncc1F)OCCc1cc(Cl)sc12] | 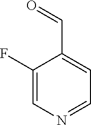 | ¹H NMR (400 MHz, DMSO-d6) δ 8.74 (s, 1H), 8.56 (s, 1H), 7.68 (s, 1H), 6.92 (s, 1H), 3.90 (d, J = 5.7 Hz, 2H), 3.11 (s, 2H), 2.98- 2.84 (m, 1H), 2.57 (d, J = 5.7 Hz, 2H), 2.13 (s, 2H), 1.92 (s, 2H), 1.44 (s, 2H), 1.16 (t, J = 7.2 Hz, 3H). LCMS m/z 367.29 [M + H]⁺. |
| 679 | 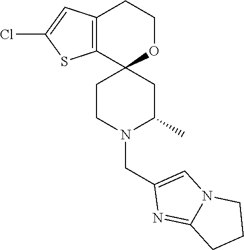 [SMILES: C[C@H]1C[C@@]2(CCN1Cc1cn3c(n1)CCC3)OCCc1cc(Cl)sc12] | 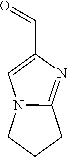 | ¹H NMR (400 MHZ, DMSO-d6) δ 7.46 (s, 1H), 6.92 (s, 1H), 4.45 (d, J = 14.2 Hz, 1H), 4.22 (d, J = 14.2 Hz, 1H), 4.05 (d, J = 7.1 Hz, 2H), 3.87 (dd, J = 11.8, 6.0 Hz, 2H), 3.42 (s, 1H), 3.28 (s, 1H), 3.16 (t, J = 12.8 Hz, 1H), 2.86 (t, J = 7.7 Hz, 2H), 2.63- 2.55 (m, 4H), 2.22 (dd, J = 27.8, 15.0 Hz, 2H), 2.02-1.86 (m, 2H), 1.42 (d, J = 6.3 Hz, 3H). LCMS m/z 378.35 [M + H]⁺. |
| 680 | 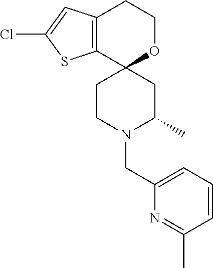 [SMILES: Cc1cccc(CN2CC[C@]3(C[C@@H]2C)OCCc2cc(Cl)sc23)n1] | 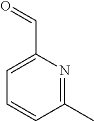 | ¹H NMR (400 MHZ, DMSO-d6) δ 10.05-9.45 (m, 1H), 7.82 (t, J = 7.7 Hz, 1H), 7.37 (dd, J = 12.4, 7.7 Hz, 2H), 6.93 (s, 1H), 4.71 (d, J = 13.8 Hz, 1H), 4.34 (d, J = 13.8 Hz, 1H), 4.00-3.84 (m, 2H), 3.26 (t, J = 12.7 Hz, 1H), 3.12 (d, J = 12.4 Hz, 1H), 2.58 (s, 2H), 2.56 (s, 3H), 2.25 (d, J = 14.7 Hz, 1H), 2.20-1.94 (m, 3H), 1.41 (d, J = 6.3 Hz, 3H). LCMS m/z 363.36 [M + H]⁺. |
| 681 | 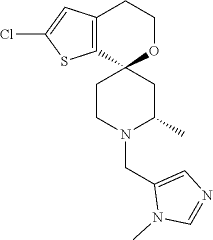 [SMILES: C[C@H]1C[C@@]2(CCN1Cc1cncn1C)OCCc1cc(Cl)sc12] | 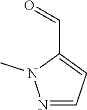 | ¹H NMR (400 MHZ, DMSO-d6) δ 7.50 (d, J = 1.9 Hz, 1H), 6.91 (s, 1H), 6.63 (d, J = 1.9 Hz, 1H), 4.75 (d, J = 14.4 Hz, 1H), 4.32 (dd, J = 14.6, 7.9 Hz, 1H), 3.96 (s, 3H), 3.88 (dp, J = 17.3, 5.8 Hz, 2H), 3.60 (s, 1H), 3.09 (d, J = 23.4 Hz, 2H), 2.58 (s, 2H), 2.36-2.09 (m, 4H), 1.51 (d, J = 6.3 Hz, 3H). LCMS m/z 352.34 [M + H]⁺. |
| 682 | 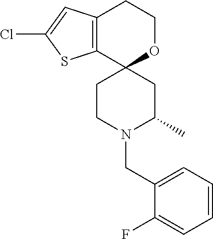 [SMILES: C[C@H]1C[C@@]2(CCN1Cc1ccccc1F)OCCc1cc(Cl)sc12] | 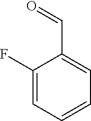 | ¹H NMR (400 MHZ, DMSO-d6) δ 9.51 (s, 1H), 7.66-7.58 (m, 2H), 7.39-7.28 (m, 2H), 6.93 (s, 1H), 4.77 (d, J = 13.4 Hz, 1H), 4.24 (s, 1H), 3.90 (dtd, J = 17.6, 11.6, 5.5 Hz, 2H), 3.63 (s, 1H), 3.19 (s, 1H), 3.07 (s, 1H), 2.59 (d, J = 5.9 Hz, 2H), 2.30 (d, J = 16.1 Hz, 1H), 2.16 (d, J = 14.9 Hz, 1H), 1.94 (d, J = 15.4 Hz, 2H), 1.48 (d, J = 6.2 Hz, 3H). LCMS m/z 366.34 [M + H]⁺. |
| 683 | 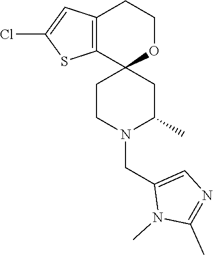 [SMILES: Cc1ncc(CN2CC[C@]3(C[C@@H]2C)OCCc2cc(Cl)sc23)n1C] | 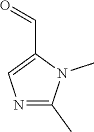 | ¹H NMR (400 MHZ, DMSO-d6) δ 7.70 (s, 1H), 6.92 (s, 1H), 3.90 (d, J = 7.8 Hz, 4H), 3.74 (s, 4H), 2.98-2.81 (m, 1H), 2.58 (s, 6H), 2.01 (d, J = 70.4 Hz, 4H), 1.41 (s, 3H). LCMS m/z 366.34 [M + H]⁺. |
| 684 | 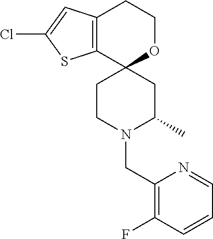 [SMILES: C[C@H]1C[C@@]2(CCN1Cc1ncccc1F)OCCc1cc(Cl)sc12] | 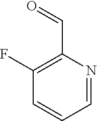 | LCMS m/z 367.33 [M + H]⁺. |
| 685 | 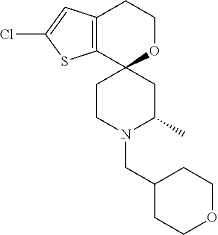 [SMILES: C[C@H]1C[C@@]2(CCN1CC1CCOCC1)OCCc1cc(Cl)sc12] | 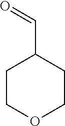 | ¹H NMR (400 MHZ, DMSO-d6) δ 6.93 (d, J = 4.4 Hz, 1H), 3.96- 3.81 (m, 4H), 3.55 (s, 1H), 3.32 (ddd, J = 12.1, 9.7, 5.3 Hz, 3H), 3.24 (d, J = 10.7 Hz, 1H), 3.16 (q, J = 11.5 Hz, 1H), 2.97-2.88 (m, 1H), 2.59 (t, J = 5.4 Hz, 2H), 2.22 (dd, J = 27.0, 14.8 Hz, 2H), 2.02 (dt, J = 36.1, 13.1 Hz, 3H), 1.75 (d, J = 12.9 Hz, 1H), 1.54 (d, J = 13.1 Hz, 1H), 1.31 (d, J = 6.4 Hz, 3H), 1.29-1.19 (m, 2H). LCMS m/z 356.36 [M + H]⁺. |
| 686 | 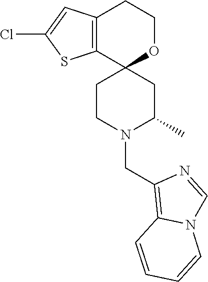 [SMILES: C[C@H]1C[C@@]2(CCN1Cc1ncn3ccccc13)OCCc1cc(Cl)sc12] | 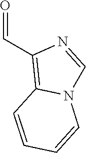 [SMILES: O=Cc1ncn2ccccc12] | LCMS m/z 388.33 [M + H]⁺. |
| 687 | 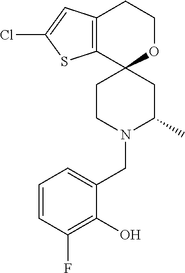 [SMILES: C[C@H]1C[C@@]2(CCN1Cc1cccc(F)c1O)OCCc1cc(Cl)sc12] | 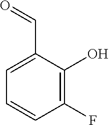 | ¹H NMR (400 MHZ, DMSO-d6) δ 10.63 (s, 1H), 9.14 (s, 1H), 7.31 (t, J = 9.6 Hz, 1H), 7.24 (d, J = 7.7 Hz, 1H), 6.92 (d, J = 5.4 Hz, 2H), 4.68 (d, J = 13.1 Hz, 1H), 4.10 (d, J = 13.1 Hz, 1H), 3.89 (dp, J = 17.2, 5.9, 5.4 Hz, 2H), 3.58 (s, 1H), 3.12 (d, J = 33.2 Hz, 2H), 2.58 (d, J = 5.9 Hz, 2H), 2.26 (d, J = 14.8 Hz, 1H), 2.13 (d, J = 14.7 Hz, 1H), 1.99 (t, J = 14.2 Hz, 2H), 1.47 (d, J = 6.2 Hz, 3H). LCMS m/z 382.28 [M + H]⁺. |
| 688 | 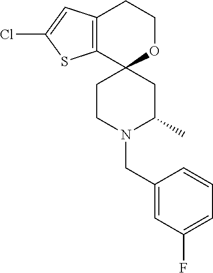 [SMILES: C[C@H]1C[C@@]2(CCN1Cc1cccc(F)c1)OCCc1cc(Cl)sc12] | 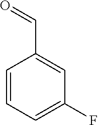 | ¹H NMR (400 MHZ, DMSO-d6) δ 7.59-7.50 (m, 1H), 7.37 (td, J = 20.0, 9.6 Hz, 3H), 6.92 (s, 1H), 4.77 (d, J = 13.0 Hz, 1H), 4.16 (s, 1H), 3.88 (dp, J = 17.3, 5.9 Hz, 2H), 3.54 (s, 1H), 3.14 (s, 1H), 3.00 (s, 1H), 2.59 (d, J = 5.9 Hz, 2H), 2.31 (d, J = 15.3 Hz, 1H), 2.15 (d, J = 15.4 Hz, 1H), 1.94 (dd, J = 24.6, 12.9 Hz, 2H), 1.48 (s, 3H). LCMS m/z 366.34 [M + H]⁺. |
| 689 | 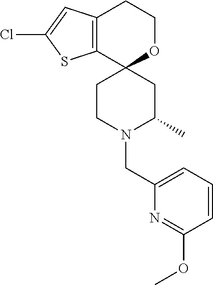 [SMILES: COc1cccc(CN2CC[C@]3(C[C@@H]2C)OCCc2cc(Cl)sc23)n1] | 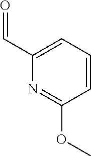 | ¹H NMR (400 MHZ, DMSO-d6) δ 7.83 (t, J = 7.8 Hz, 1H), 7.20 (d, J = 7.2 Hz, 1H), 6.96-6.82 (m, 2H), 4.59 (d, J = 13.9 Hz, 1H), 4.43 (s, 1H), 3.91 (s, 3H), 3.87 (q, J = 5.9 Hz, 2H), 3.67 (s, 1H), 2.58 (d, J = 5.8 Hz, 2H), 2.24 (dd, J = 28.5, 14.8 Hz, 2H), 2.00 (d, J = 15.4 Hz, 2H), 1.44 (d, J = 6.3 Hz, 3H). LCMS m/z 379.34 [M + H]⁺. |
| 690 | 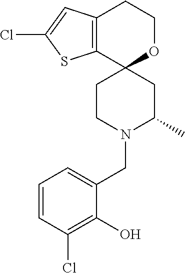 [SMILES: C[C@H]1C[C@@]2(CCN1Cc1cccc(Cl)c1O)OCCc1cc(Cl)sc12] | 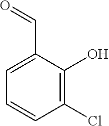 | ¹H NMR (400 MHZ, DMSO-d6) δ 10.29 (s, 1H), 7.49 (d, J = 8.0 Hz, 1H), 7.38 (d, J = 7.6 Hz, 1H), 6.96 (t, J = 7.8 Hz, 1H), 6.92 (s, 1H), 4.69 (s, 1H), 4.09 (s, 1H), 3.89 (dp, J = 17.3, 5.9 Hz, 2H), 3.42 (d, J = 41.8 Hz, 2H), 3.15 (s, 1H), 3.00 (s, 1H), 2.58 (d, J = 6.5 Hz, 2H), 2.25 (d, J = 14.8 Hz, 1H), 2.12 (d, J = 14.8 Hz, 1H), 1.98 (t, J = 13.4 Hz, 2H), 1.53-1.37 (m, 3H). LCMS m/z 398.26 [M + H]⁺. |
| 691 | 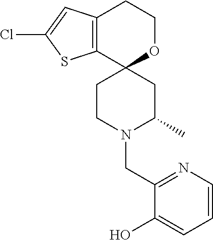 [SMILES: C[C@H]1C[C@@]2(CCN1Cc1ncccc1O)OCCc1cc(Cl)sc12] | 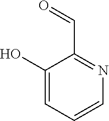 | ¹H NMR (400 MHZ, DMSO-d6) δ 10.85 (s, 1H), 8.16 (dd, J = 3.9, 2.1 Hz, 1H), 7.39-7.31 (m, 2H), 6.92 (s, 1H), 4.64 (d, J = 14.5 Hz, 1H), 4.36 (d, J = 14.4 Hz, 1H), 3.89 (qt, J = 11.6, 5.5 Hz, 2H), 3.71 (s, 1H), 3.27 (d, J = 8.1 Hz, 2H), 2.58 (t, J = 5.6 Hz, 2H), 2.28-2.00 (m, 4H), 1.38 (d, J = 6.3 Hz, 3H). LCMS m/z 365.34 [M + H]⁺ |
| 692 | 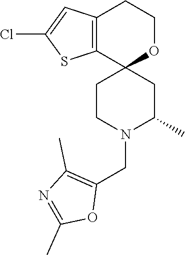 [SMILES: Cc1nc(C)c(CN2CC[C@]3(C[C@@H]2C)OCCc2cc(Cl)sc23)o1] | 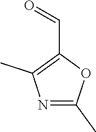 | ¹H NMR (400 MHZ, DMSO-d6) δ 9.96 (s, 1H), 6.93 (s, 1H), 4.65 (d, J = 15.0 Hz, 1H), 4.47 (d, J = 15.3 Hz, 1H), 3.87 (dt, J = 12.4, 5.8 Hz, 2H), 3.24 (d, J = 12.2 Hz, 1H), 3.11 (s, 1H), 2.61-2.56 (m, 2H), 2.40 (s, 3H), 2.25 (dd, J = 29.2, 14.9 Hz, 2H), 2.14 (s, 3H), 1.93 (q, J = 13.7, 13.1 Hz, 2H), 1.42 (d, J = 6.3 Hz, 3H). LCMS m/z 367.33 [M + H]⁺. |
| 693 | 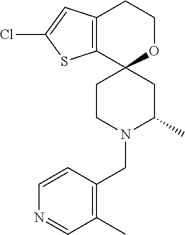 [SMILES: Cc1cnccc1CN1CC[C@]2(C[C@@H]1C)OCCc1cc(Cl)sc12] | 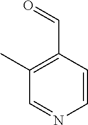 | ¹H NMR (400 MHZ, DMSO-d6) δ 8.56 (d, J = 11.6 Hz, 2H), 7.56 (s, 1H), 6.93 (s, 1H), 4.79 (s, 1H), 4.25 (s, 1H), 3.94-3.89 (m, 3H), 3.25 (s, 1H), 2.93 (q, J = 6.8 Hz, 1H), 2.60 (s, 2H), 2.42 (s, 3H), 2.38-2.20 (m, 1H), 2.04 (d, J = 50.1 Hz, 3H), 1.48 (s, 3H). LCMS m/z 363.31 [M + H]⁺. |

Footnotes
¹Second Step: Deprotection of TBS group using HCl in methanol
2. Second step: SnAr using cyclopropanamine (2.5 eq), potassium carbonate (4 eq) in DMF (0.5 mL) at 90° C. overnight. Reaction was diluted in DCM and water and aqueous layer was collected through a phase separator. Purification by reversed-phase HPLC. Method: C18 Waters Sunfire column (30 x 150 mm, 5 micron). Gradient: MeCN in H2O with 5 mM HCl followed by Purification by reversed-phase HPLC. Method: C18 Waters Sunfire column (30 x 150 mm, 5 micron). Gradient: MeCN in H2O with 10 mM Ammonium Hydroxide yielded the product.

### Preparation of S50

### (2′S, 7R)-2-chloro-2′-methyl-spiro[4,5-dihydrothieno[2,3-c]pyran-7,4′-piperidine](S50)

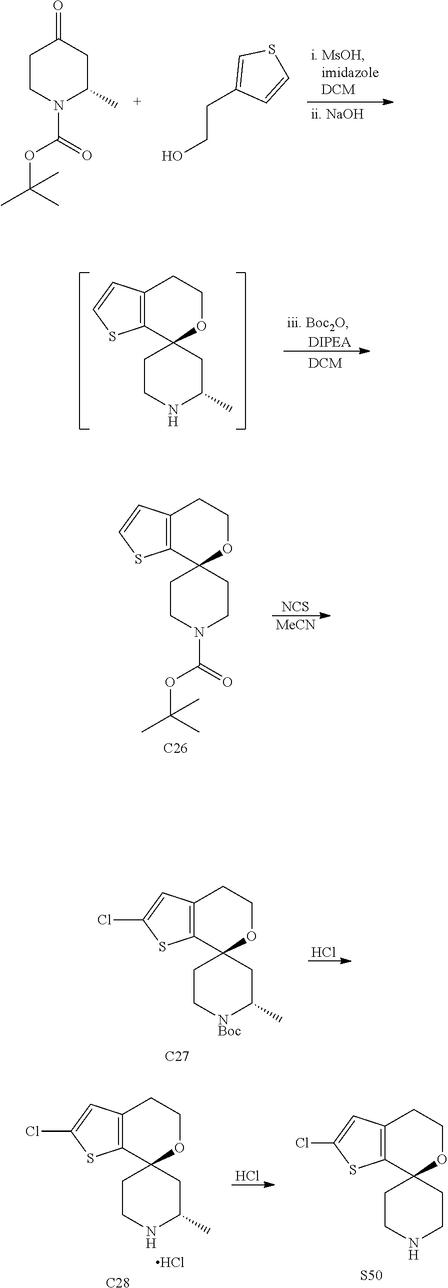

### Step 1(i): Synthesis of tert-butyl (2S)-2-methyl-4-oxo-piperidine-1-carboxylate

To a mixture of tert-butyl (2S)-2-methyl-4-oxo-piperidine-1-carboxylate (25 g, 117.2 mmol) in DCM (500 mL) was added MsOH (24 mL, 369.8 mmol) and the mixture was stirred at room temperature for 40 min. The mixture was vacuum-nitrogen purged five times to remove dissolved isobutylene.

### Step 1(ii): Synthesis of (2′S, 7R)-2′-methylspiro[4,5-dihydrothieno[2,3-c]pyran-7,4′-piperidine]

To the mixture was then added 2-(3-thienyl)ethanol (16.5 g, 128.7 mmol) and the reaction stirred overnight. The mixture was concentrated to about 50 mL and rediluted with water (300 mL) and diethyl ether (600 mL). The organic layer was removed. The aqueous layer was adjusted to pH 14 with aqueous NaOH (64 mL of 6 M, 384.0 mmol), and then DCM (600 mL) was added to extract the free base. The organic layer was washed with pH 14 water (2×200 mL), pH 14 brine (200 mL), dried with MgSO₄, filtered, and concentrated to yield (2′S,7R)-2′-methylspiro[4,5-dihydrothieno[2,3-c]pyran-7,4′-piperidine](24900 mg, 95%) LCMS m/z 224.54 [M+H]⁺.

### Step 1(iii): Synthesis of tert-butyl (2′S, 7R)-2′-methylspiro[4,5-dihydrothieno[2,3-c]pyran-7,4′-piperidine]-1′-carboxylate (C26)

To a mixture of the product from the first two steps in DCM (500 mL) was added DIPEA (20 mL, 114.8 mmol) followed by Boc₂O (27 mL, 117.5 mmol). The reaction stirred for 45 min. Imidazole (1 g, 14.69 mmol) was added to quench excess Boc₂O. Then 2 N HCl (100 mL) was added and the mixture was stirred for 20 min. The layers were separated, and the organic layer was washed with 1 N HCl (100 mL). The organic layer was passed over a phase separator and concentrated to yield tert-butyl (2′S,7R)-2′-methylspiro[4,5-dihydrothieno[2,3-c]pyran-7,4′-piperidine]-1′-carboxylate (37300 mg, 91%) as a yellow oil. LCMS m/z 323.11 [M+H]⁺.

### Step 2: Synthesis of tert-butyl (2′S, 7R)-2-chloro-2′-methyl-spiro[4,5-dihydrothieno[2,3-c]pyran-7,4′-piperidine]-1′-carboxylate (C27)

Tert-butyl (2′S,7R)-2′-methylspiro[4,5-dihydrothieno[2,3-c]pyran-7,4′-piperidine]-1′-carboxylate C26 (16 g, 49.47 mmol) was dissolved in acetonitrile (150 mL) and NCS (13.2 g, 98.85 mmol) was added. The reaction was as heated at 65° C. and stirred overnight. The reaction was diluted with 1N NaOH/EtOAc washed with sodium thiosulfate solution, brine and dried over Na₂SO₄ and concentrated to an oil. Purification by silica gel chromatography (Gradient: 0-30% EtOAc in heptane) yielded the product. tert-butyl (2′S,7R)-2-chloro-2′-methyl-spiro[4,5-dihydrothieno[2,3-c]pyran-7,4′-piperidine]-1′-carboxylate (15.4 g, 83%). LCMS m/z 358.14 [M+H]⁺.

### Step 3: Synthesis of (2′S, 7R)-2-chloro-2′-methyl-spiro[4,5-dihydrothieno[2,3-c]pyran-7,4′-piperidine](Hydrochloride salt) (C28)

A mixture of tert-butyl (2′S,7R)-2-chloro-2′-methyl-spiro[4,5-dihydrothieno[2,3-c]pyran-7,4′-piperidine]-1′-carboxylate (9500 mg, 26.37 mmol) in a solution of HCl (25 mL of 4 M, 100.0 mmol) in dioxane was stirred at room temperature overnight. After stirring overnight, the mixture had congealed into a solid mass. To the mixture was added diethyl ether (200 mL total), the mixture was filtered and rinsed with additional ether and dried to yield (2′S,7R)-2-chloro-2′-methyl-spiro[4,5-dihydrothieno[2,3-c]pyran-7,4′-piperidine](Hydrochloride salt) (C28) (7.4757 g, 94%). ¹H NMR (400 MHz, DMSO-d6) δ 9.25-8.93 (m, 2H), 6.92 (s, 1H), 3.96-3.81 (m, 2H), 3.38-3.26 (m, 1H), 3.23-3.12 (m, 1H), 3.04 (q, J=12.2 Hz, 1H), 2.58 (t, J=5.5 Hz, 2H), 2.13 (t, J=15.0 Hz, 2H), 2.01 (td, J=14.1, 13.7, 4.6 Hz, 1H), 1.84 (dd, J=14.3, 12.1 Hz, 1H), 1.25 (d, J=6.5 Hz, 3H). LCMS m/z 258.09 [M+H]⁺.

### Step 4: Synthesis of (2′S, 7R)-2-chloro-2′-methyl-spiro[4,5-dihydrothieno[2,3-c]pyran-7,4′-piperidine](S50)

(2′S,7R)-2-chloro-2′-methyl-spiro[4,5-dihydrothieno[2,3-c]pyran-7,4′-piperidine] (2.70 g) was dissolved in water and to the mixture was added sat. sodium bicarbonate, followed by NaOH to adjust to pH >10. At this time, the product had oiled out and was dissolved in DCM (10 mL) and the aqueous layer was extracted with additional DCM (10 mL). The combined organic layers were dried over Na₂SO₄, filtered, and concentrated to yield (2′S,7R)-2-chloro-2′-methyl-spiro[4,5-dihydrothieno[2,3-c]pyran-7,4′-piperidine]S50 (2280 mg, 93%). ¹H NMR (400 MHz, Chloroform-d) δ 6.57 (s, 1H), 3.98-3.81 (m, 2H), 3.18-3.00 (m, 2H), 2.94 (ddd, J=12.2, 4.8, 2.1 Hz, 1H), 2.67-2.51 (m, 2H), 2.07-1.94 (m, 2H), 1.66 (d, J=4.8 Hz, 1H), 1.38 (dd, J=13.7, 11.4 Hz, 1H), 1.09 (d, J=6.4 Hz, 3H). LCMS m/z 258.09 [M+H]⁺.

### Preparation of S51

### Preparation of (2′S, 7S)-2-chloro-2′-methyl-spiro[4,5-dihydrothieno[2,3-c]pyran-7,4′-piperidine](S51)

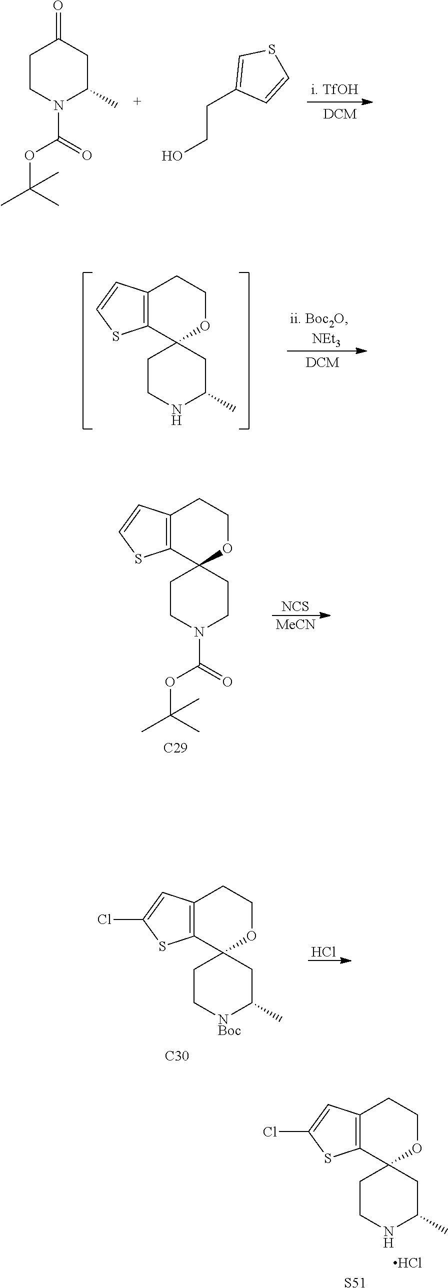

### Step 1(i): Synthesis of (2′S)-2′-methylspiro[4,5-dihydrothieno[2,3-c]pyran-7,4′-piperidine]

A solution of 2-(3-thienyl)ethanol (1.275 g, 9.946 mmol) and tert-butyl (2S)-2-methyl-4-oxo-piperidine-1-carboxylate (2.217 g, 10.40 mmol) in DCM (18 mL) was cooled to −78° C. To the reaction trifluoromethanesulfonic acid (1.5 mL, 16.95 mmol) was added and the reaction was stirred at −78° C. for 1.5 hours. The reaction was quenched into a biphasic mixture of sat. NaHCO₃ (75 mL) and DCM (50 mL) and the aqueous layer pH was adjusted to >10 with 2N NaOH. The aqueous layer was then extracted with DCM (3×75 mL) and filtered through an SPE cartridge and concentrated to give crude (2′S)-2′-methylspiro[4,5-dihydrothieno[2,3-c]pyran-7,4′-piperidine].

### Step 1(ii): Synthesis of tert-butyl (2′S, 7S)-2′-methylspiro[4,5-dihydrothieno[2,3-c]pyran-7,4′-piperidine]-1′-carboxylate (C29)

Dissolved the crude from previous step in DCM (20 mL) and added triethylamine (3.1 mL, 22.24 mmol) followed by tert-butoxycarbonyl tert-butyl carbonate (3.1 mL, 13.49 mmol). The reaction was stirred at room temperature for 18 hours. The solution was diluted with DCM and washed with water (50 mL). The aqueous layer was extracted with DCM (2×50 mL), dried over Na₂SO₄, filtered through an SPE filter, and concentrated under vacuum. Purification by silica gel chromatography (0 to 30% EtOAc in heptane) yielded tert-butyl (2′S,7S)-2′-methylspiro[4,5-dihydrothieno[2,3-c]pyran-7,4′-piperidine]-1′-carboxylate C29 (2.95 g, 92%). ¹H NMR (300 MHz, Methanol-d₄) δ 7.21 (d, J=5.1 Hz, 1H), 6.77 (d, J=5.1 Hz, 1H), 4.37 (p, J=7.0 Hz, 1H), 4.03-3.84 (m, 3H), 3.39-3.21 (m, 1H), 2.68 (td, J=5.5, 2.6 Hz, 2H), 2.16-1.65 (m, 4H), 1.48 (s, 9H), 1.34 (d, J=7.2 Hz, 3H).

### Step 2: Synthesis of tert-butyl (2′S, 7S)-2-chloro-2′-methyl-spiro[4,5-dihydrothieno[2,3-c]pyran-7,4′-piperidine]-1′-carboxylate (C30)

To a solution of tert-butyl (2′S,7S)-2′-methylspiro[4,5-dihydrothieno[2,3-c]pyran-7,4′-piperidine]-1′-carboxylate C29 (239 mg, 0.7389 mmol) in MeCN (3 mL) at room temperature was added NCS (138 mg, 1.033 mmol) followed by DMAP (5 mg, 0.04093 mmol). The reaction was heated to 55° C. for 24 hours. The reaction was cooled to rt and stirred overnight, then reaction was quenched with 15% aq sodium bisulfite (10 mL). Stirred for 15 min, then partitioned between brine (20 mL) and DCM (20 mL). Aqueous layer was separated and extracted with DCM (2×20 mL), the pooled organic layers were dried over Na₂SO₄, filtered through a phase separator, and concentrated. Purification by silica gel chromatography (0 to 30% EtOAc in heptane) afforded a crude mixture with some starting material remaining.

The crude material was resubjected to the reaction conditions by dissolving in MeCN (2.5 mL) and N-chlorosuccinimide (50 mg, 0.3744 mmol) and DMAP (5 mg, 0.04093 mmol) were added. The reaction was heated to 60° C. for 6 hours, then cooled to room temperature and quenched with 15% aq sodium bisulfite (10 mL). Stirred for 15 min, then partitioned between brine and DCM (20 mL each). Aqueous layer was separated and extracted with DCM (2×20 mL), the pooled organic layers were dried over Na₂SO₄, filtered through a phase separator, and concentrated. Purification by silica gel chromatography (0 to 30% EtOAc in heptane) to afford tert-butyl (2′S,7S)-2-chloro-2′-methyl-spiro[4,5-dihydrothieno[2,3-c]pyran-7,4′-piperidine]-1′-carboxylate C30 (170 mg, 63%). ¹H NMR (300 MHz, Methanol-d₄) δ 6.66 (s, 1H), 4.35 (q, J=6.9 Hz, 1H), 4.03-3.85 (m, 3H), 3.27 (s, 1H), 2.69-2.50 (m, 2H), 2.14-1.94 (m, 2H), 1.80 (dd, J=14.5, 6.6 Hz, 1H), 1.68 (ddd, J=13.7, 12.9, 4.7 Hz, 1H), 1.47 (s, 9H), 1.32 (d, J=7.1 Hz, 3H).

### Step 3: Synthesis of (2′S, 7S)-2-chloro-2′-methyl-spiro[4,5-dihydrothieno[2,3-c]pyran-7,4′-piperidine](S51)

To a stirred solution of tert-butyl (2′S,7S)-2-chloro-2′-methyl-spiro[4,5-dihydrothieno[2,3-c]pyran-7,4′-piperidine]-1′-carboxylate C30 (170 mg, 0.4750 mmol) in dioxane (2 mL) was added HCl (600 μL of 4 M, 2.400 mmol) in dioxane and the reaction was stirred for 20 hours at room temperature. The reaction was concentrated. Purification by reversed-phase HPLC. Method: C18 Waters Sunfire column (30×150 mm, 5 micron). Gradient: MeCN in H₂O with 5 mM HCl and lyopholization afforded the product (2′S,7S)-2-chloro-2′-methyl-spiro[4,5-dihydrothieno[2,3-c]pyran-7,4′-piperidine]S51 (Hydrochloride salt) (108.2 mg, 75%). ¹H NMR (300 MHz, Methanol-d₄) δ 6.77 (s, 1H), 3.98 (t, J=5.8 Hz, 2H), 3.72 (h, J=6.3 Hz, 1H), 3.53 (ddd, J=13.8, 9.7, 4.0 Hz, 1H), 3.29-3.16 (m, 1H), 2.70-2.60 (m, 2H), 2.25-2.05 (m, 4H), 1.52 (d, J=7.0 Hz, 3H). LCMS m/z 258.0 [M+H]⁺.

### Preparation of C31

### 1-methylprop-2-ynyl 4-methylbenzenesulfonate (C31)

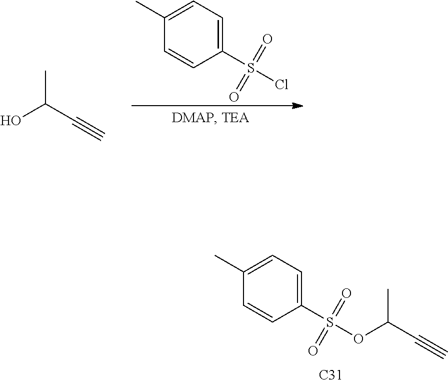

### Synthesis of 1-methylprop-2-ynyl 4-methylbenzenesulfonate (C31)

To a stirred solution of but-3-yn-2-ol (1.5 g, 0.0214 mol) in DCM (15 mL) at 0° C., DMAP (1 g, 0.0082 mol), TEA (726.00 mg, 1 mL, 0.0072 mol) and 4-methylbenzenesulfonyl chloride (0.45 g, 0.0024 mol) were added. The reaction mixture was stirred for 30 min at 0° C. and 1 hour at room temperature. Then the reaction mixture was filtered. The filtrate was washed with saturated CuSO₄ solution (200 mL), saturated NaHCO₃ solution (200 mL) and brine (200 mL). The organic layer was dried over Na₂SO₄ and concentrated under reduced pressure. Purification by silica gel chromatography (gradient: 10-30% EtOAc in pet ether) afforded 1-methylprop-2-ynyl 4-methylbenzenesulfonate C31 (1.2 g, 25%). ¹H NMR (400 MHz, Chloroform-d) δ ppm 7.83-7.80 (m, 2H), 7.34-7.26 (m, 2H), 5.19-5.13 (m, 1H), 2.44-2.41 (m, 4H), 1.58-1.56 (m, 3H).

### Compound 694

### (2′S, 7R)-2-chloro-2‘-methyl-’-(1-methylprop-2-ynyl)spiro[4,5-dihydrothieno[2,3-c]pyran-7,4′-piperidine](694)

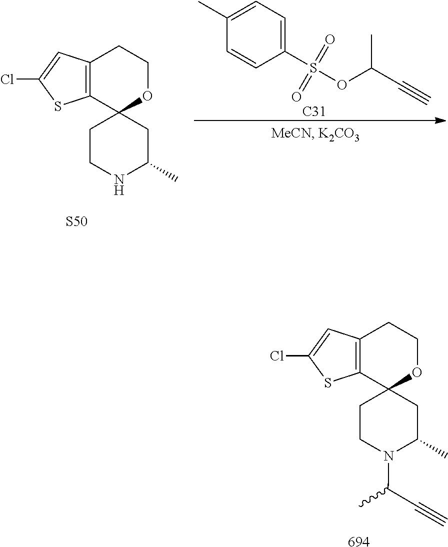

### Preparation of (2′S, 7R)-2-chloro-2′-methyl-1′-(1-methylprop-2-ynyl)spiro[4,5-dihydrothieno[2,3-c]pyran-7,4′-piperidine](694)

To a stirred solution of (2′S,7R)-2-chloro-2′-methyl-spiro[4,5-dihydrothieno[2,3-c]pyran-7,4′-piperidine](Trifluoroacetic Acid (1)) (S50) (500 mg, 0.0012 mol) in MeCN (10 mL), K₂CO₃ (415 mg, 0.0030 mol) and 1-methylprop-2-ynyl 4-methylbenzenesulfonate (C₃₁) (403 mg, 0.0018 mol) were added at room temperature. The reaction mixture was stirred for 16 h at 80° C. The reaction mixture was cooled to room temperature and concentrated under reduced pressure. The reaction mixture was cooled to room temperature and quenched with water (50 ml) and extracted with EtOAc (2×100 mL). The combined organic layers were dried over Na₂SO₄, filtered and concentrated under reduced pressure. Purification by silica gel chromatography (0-30% EtOAc in hexanes). followed by purification by reversed-phase chromatography (Column: -xselectPhenyl hexyle (250*19) mm, 5 u. Gradient: 0-20% MeCN in water with 0.1% formic acid) afforded (2′S,7R)-2-chloro-2′-methyl-1′-(1-methylprop-2-ynyl)spiro[4,5-dihydrothieno[2,3-c]pyran-7,4′-piperidine]694 (28 mg, 8%). ¹H NMR (400 MHz, DMSO-d6) δ ppm 6.84 (s, 1H), 3.99-3.96 (m, 1H), 3.86-3.82 (m, 2H), 3.11-3.11 (m, 1H), 2.93-2.90 (m, 1H), 2.69-2.67 (m, 1H), 2.58-2.49 (m, 2H), 2.01-1.90 (m, 2H), 1.64-1.62 (m, 1H), 1.44-1.38 (m, 1H), 1.11 (d, J=6.8 Hz, 3H), 0.98 (d, J=6.4 Hz, 3H). LCMS m/z 310.0 [M+H]⁺.

### Compound 695

### (2′S,7S)-2-chloro-2′-methyl-1′-[[1-(2-methylsulfonylethyl)triazol-4-yl]methyl]spiro[4,5-dihydrothieno[2,3-c]pyran-7,4′-piperidine](695)

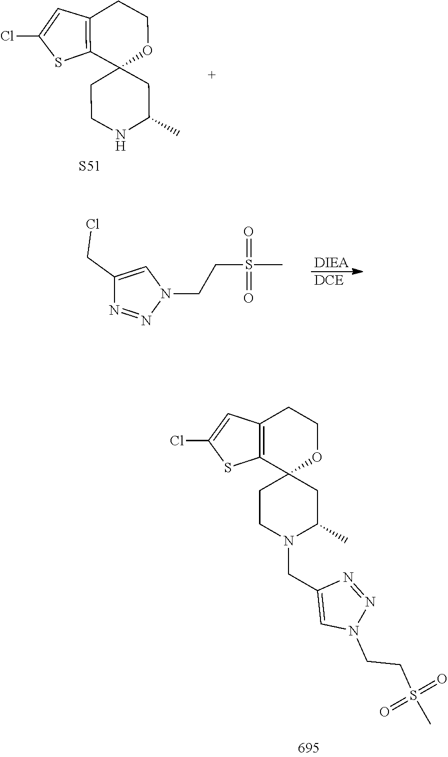

### Preparation of (2′S, 7S)-2-chloro-2′-methyl-1′-[[1-(2-methylsulfonylethyl)triazol-4-yl]methyl]spiro[4,5-dihydrothieno[2,3-c]pyran-7,4′-piperidine](695)

(2′S,7S)-2-chloro-2′-methyl-spiro[4,5-dihydrothieno[2,3-c]pyran-7,4′-piperidine](35 mg, 0.1358 mmol) (S51) was dissolved in DCE (1 mL) and DIEA (50 μL, 0.2871 mmol) and 4-(chloromethyl)-1-(2-methylsulfonylethyl)triazole (34 mg, 0.1520 mmol) were added. The reaction mixture was heated in a sealed tube at 60° C. overnight. Additional 4-(chloromethyl)-1-(2-methylsulfonylethyl)triazole (10 mg, 0.04471 mmol) was added and the reaction was heated overnight at 60° C. The reaction mixture was concentrated. Purification by reversed-phase HPLC. Method: C18 Waters Sunfire column (30×150 mm, 5 micron). Gradient: MeCN in H₂O with 0.1% trifluoroacetic acid afford (2′S,7S)-2-chloro-2′-methyl-1′-[[1-(2-methylsulfonylethyl)triazol-4-yl]methyl]spiro[4,5-dihydrothieno[2,3-c]pyran-7,4′-piperidine](695). (Trifluoroacetate salt) (32 mg, 41%). ¹H NMR (300 MHz, Chloroform-d) δ 8.26 (s, 1H), 6.60 (s, 1H), 4.94 (t, J=6.8 Hz, 2H), 4.41 (d, J=19.1 Hz, 2H), 3.93 (t, J=5.1 Hz, 2H), 3.81-3.65 (m, 3H), 3.56-3.37 (m, 2H), 2.95 (s, 3H), 2.69-2.58 (m, 2H), 2.38-2.09 (m, 4H), 1.70 (d, J=7.1 Hz, 3H). LCMS m/z 445.31 [M+H]⁺.

### Compound 696

### (2′S, 7R)-2-chloro-2′-methyl-1′-[[1-(2-methylsulfonylethyl)triazol-4-yl]methyl]spiro[4,5-dihydrothieno[2,3-c]pyran-7,4-piperidine](696)

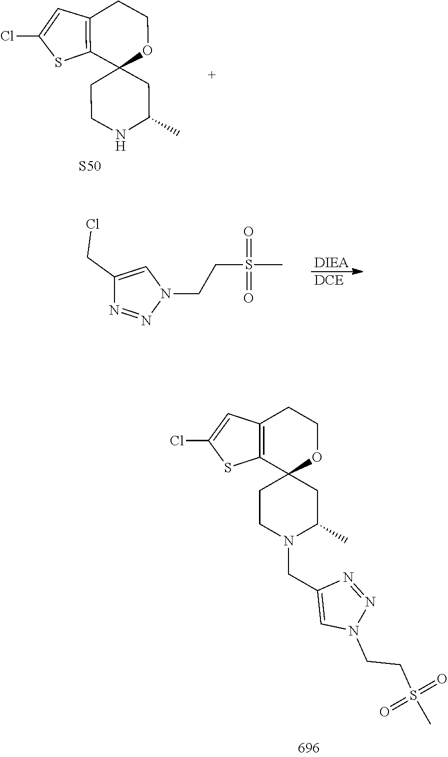

### Synthesis of 2′S)-2-chloro-2′-methyl-1′-[[1-(2-methylsulfonylethyl)triazol-4-yl]methyl]spiro[4,5-dihydrothieno[2,3-c]pyran-7,4′-piperidine](696)

(2′S,7R)-2-chloro-2′-methyl-spiro[4,5-dihydrothieno[2,3-c]pyran-7,4′-piperidine](48 mg, 0.1862 mmol) was dissolved in DCE (1 mL) and 4-(chloromethyl)-1-(2-methylsulfonylethyl)triazole (45 mg, 0.2012 mmol) and DIEA (65 μL, 0.3732 mmol) were added. The reaction mixture was heated to 60° C. overnight in a sealed tube. Additional 4-(chloromethyl)-1-(2-methylsulfonylethyl)triazole (15 mg, 0.06706 mmol) added and heating continued for 24 hrs. The reaction mixture was concentrated. Purification by reversed-phase HPLC. Method: C18 Waters Sunfire column (30×150 mm, 5 micron). Gradient: MeCN in H₂O with 0.1% trifluoroacetic acid afforded (2′S)-2-chloro-2‘-methyl-’-[[1-(2-methylsulfonylethyl)triazol-4-yl]methyl]spiro[4,5-dihydrothieno[2,3-c]pyran-7,4′-piperidine](Trifluoroacetate salt) (696) (46 mg, 43%). ¹H NMR (300 MHz, Chloroform-d) δ 8.28 (s, 1H), 6.61 (s, 1H), 4.97 (td, J=6.6, 4.0 Hz, 2H), 4.78 (d, J=14.0 Hz, 1H), 4.26 (d, J=14.0 Hz, 1H), 3.97-3.65 (m, 4H), 3.65-3.55 (m, 1H), 3.40-3.18 (m, 2H), 2.95 (s, 3H), 2.69-2.56 (m, 2H), 2.28-2.09 (m, 4H), 1.62 (d, J=6.5 Hz, 3H). LCMS m/z 445.21 [M+H]⁺.

### Compound 697

### (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine](697)

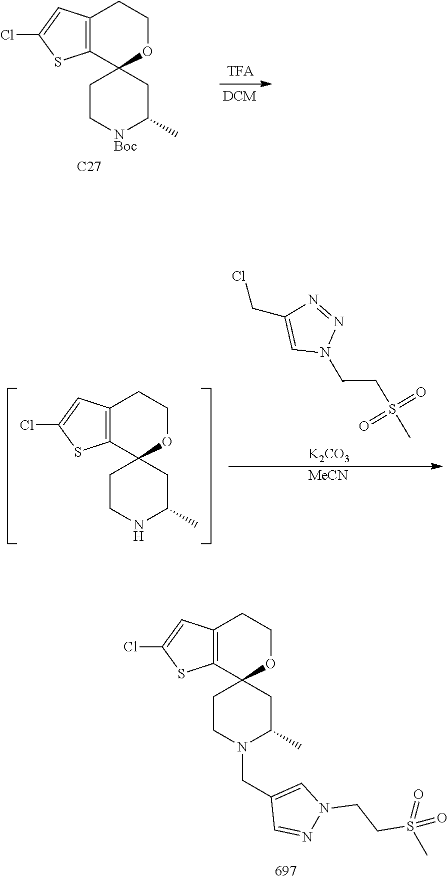

### Preparation of (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine](697)

i. To a solution of tert-butyl (2′S,7R)-2-chloro-2′-methyl-spiro[4,5-dihydrothieno[2,3-c]pyran-7,4′-piperidine]-1′-carboxylate (65 mg, 0.1780 mmol) in DCM (1 mL) was added TFA (200 μL, 2.596 mmol), and the mixture was refluxed for 5 minutes. At this time, UPLC indicated complete conversion and the mixture was concentrated to dryness, diluted in DCM and concentrated again.

ii. The oil from the first step was diluted with acetonitrile (1.5 mL) and K₂CO₃ (75 mg, 0.5427 mmol) was added followed by 4-(chloromethyl)-1-(2-methylsulfonylethyl)pyrazole (Hydrochloride salt) (90 mg, 0.3473 mmol). The mixture was stirred at 70° C. The mixture was stirred overnight. The mixture was cooled to room temperature and diluted with water (10 mL) and EtOAc (15 mL). The phases were separated, and the organic layer was washed with water (2×10 mL) and brine (10 mL), and the organic layer was dried with MgSO₄, filtered, and concentrated. Purification by silica gel chromatography (0-20% MeOH in DCM). afforded (2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine](697) (41.6 mg, 51%). ¹H NMR (400 MHz, Chloroform-d) δ 7.51 (d, J=0.7 Hz, 1H), 7.47 (s, 1H), 6.57 (s, 1H), 4.64-4.56 (m, 2H), 3.89-3.75 (m, 3H), 3.71-3.59 (m, 3H), 2.69-2.43 (m, 8H), 2.00 (tt, J=14.2, 2.9 Hz, 2H), 1.82 (td, J=13.3, 4.6 Hz, 1H), 1.62 (dd, J=13.8, 11.4 Hz, 1H), 1.20 (d, J=6.2 Hz, 3H). LCMS m/z 444.04 [M+H]⁺.

### Compound 698

### (2′S, 7R)-2-chloro-2′-methyl-1′-(1H-pyrazol-4-ylmethyl)spiro[4,5-dihydrothieno[2,3-c]pyran-7,4′-piperidine] (698)

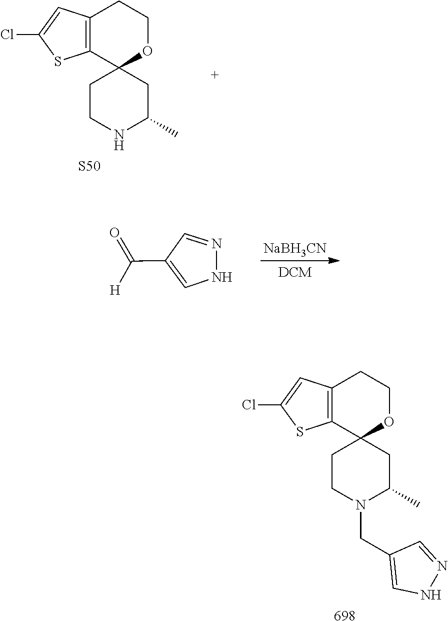 [SMILES: C[C@H]1C[C@@]2(CCN1)OCCc1cc(Cl)sc12 ; "S50" ; O=Cc1cn[nH]c1 ; "NaBH3CN" ; C[C@H]1C[C@@]2(CCN1Cc1cn[nH]c1)OCCc1cc(Cl)sc12 ; "698"]

### Preparation of (2′S, 7R)-2-chloro-2′-methyl-1′-(1H-pyrazol-4-ylmethyl)spiro[4,5-dihydrothieno[2,3-c]pyran-7,4′-piperidine] (698)

A mixture of 1H-pyrazole-4-carbaldehyde (150 mg, 1.561 mmol), (2′S,7R)-2-chloro-2′-methyl-spiro[4,5-dihydrothieno[2,3-c]pyran-7,4′-piperidine]S50 (Trifluoromethanesulfonate) (350 mg, 0.8603 mmol), cyanoboranuide (Sodium salt) (1000 mg of 2 mmol/g, 2.000 mmol), in DCM (10 mL) was stirred at 110° C. The mixture was cooled to room temperature, filtered and concentrated under reduced pressure. Purification by silica gel chromatography (0-20% MeOH in DCM) to afford the product. (2′S,7R)-2-chloro-2′-methyl-1′-(1H-pyrazol-4-ylmethyl)spiro[4,5-dihydrothieno[2,3-c]pyran-7,4′-piperidine](72 mg, 25%). ¹H NMR (400 MHz, Chloroform-d) δ 7.56 (s, 2H), 6.50 (s, 1H), 4.05 (s, 1H), 3.88-3.71 (m, 3H), 3.70-3.57 (m, 1H), 2.82 (s, 2H), 2.66-2.47 (m, 4H), 2.01-1.91 (m, 3H), 1.28 (d, J=6.3 Hz, 3H). LCMS m/z 337.88 [M+H]⁺.

### Compound 699

### (2S,7R)-2′-chloro-1-((1-(2-methoxyethyl)-1H-pyrazol-4-yl)methyl)-2-methyl-4′,5′-dihydrospiro[piperidine-4,7′-thieno[2,3-c]pyran] (699)

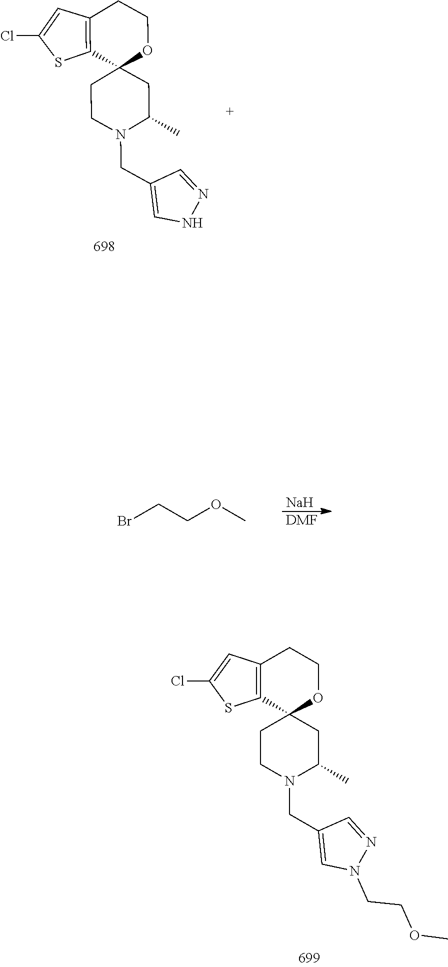

### Preparation of (2S,7R)-2′-chloro-1-((1-(2-methoxyethyl)-1H-pyrazol-4-yl)methyl)-2-methyl-4′,5′-dihydrospiro[piperidine-4,7′-thieno[2,3-c]pyran] (699)

To a mixture of (2′S,7R)-2-chloro-2′-methyl-1′-(1H-pyrazol-4-ylmethyl)spiro[4,5-dihydrothieno[2,3-c]pyran-7,4′-piperidine]698 (20 mg) in DMF (1 mL) was added NaH (3 mg, 60% w/w) followed by 1-bromo-2-methoxy-ethane (16 mL). The reaction stirred at room temperature for 80 min and was then heated to 60° C. and stirred for 5 hours. Purification by reversed-phase HPLC. Method: C18 Waters Sunfire column (30×150 mm, 5 micron). Gradient: MeCN in H₂O with 5 mM HCl afforded the product (HCl salt) EA #6 (18.8 mg, 73.3%). ¹H NMR (400 MHz, DMSO-d6) δ 10.94 (s, 1H), 7.95 (s, 1H), 7.65 (s, 1H), 6.91 (s, 1H), 4.35 (d, J=14.3 Hz, 1H), 4.28 (t, J=5.3 Hz, 2H), 4.18 (dd, J=14.0, 5.3 Hz, 1H), 3.82 (hept, J=6.0 Hz, 2H), 3.68 (t, J=5.2 Hz, 2H), 3.21 (s, 5H), 3.16 (d, J=14.3 Hz, 1H), 2.98 (d, J=15.3 Hz, 1H), 2.56 (s, 1H), 2.18 (q, J=14.1, 13.1 Hz, 4H), 1.45 (d, J=6.3 Hz, 3H). LCMS m/z 396.14 [M+H]⁺.

### Compounds 700-707

Compounds 700-707 (see Table 30) were prepared in a single step from an alkylation of 698 and the relevant alkyl halide as described for compound 699. Alkyl halides were obtained from commercial sources. Any modifications to methods are noted in Table 30 and accompanying footnotes.

TABLE 30
Preparation method, structure and physiochemical data for compounds 700-707

| | | | | ¹H NMR; |
|---|---|---|---|---|
| Cmpd | Structure | Alkyl halide | Method | LCMS m/z [M + H]⁺ |
| 700 | 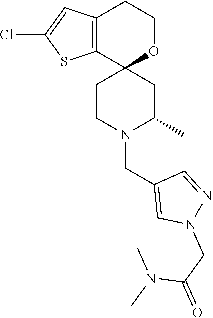 [SMILES: C[C@H]1C[C@@]2(CCN1Cc1cnn(CC(=O)N(C)C)c1)OCCc1cc(Cl)sc12] | 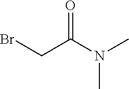 | 699² | LCMS m/z 423.36 [M + H]⁺. |
| 701 | 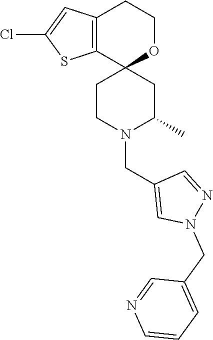 [SMILES: C[C@H]1C[C@@]2(CCN1Cc1cnn(Cc3cccnc3)c1)OCCc1cc(Cl)sc12] | 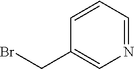 | 699 | ¹H NMR (400 MHz, DMSO-d6) δ 11.32 (s, 1H), 8.69 (d, J = 7.9 Hz, 2H), 8.18 (s, 1H), 8.00 (s, 1H), 7.72 (d, J = 7.6 Hz, 2H), 6.91 (s, 1H), 5.54 (s, 2H), 4.37 (d, J = 13.9 Hz, 1H), 4.17 (dd, J = 14.1, 5.5 Hz, 2H), 3.84 (q, J = 6.1 Hz, 3H), 3.26 (s, 1H), 2.99 (d, J = 6.4 Hz, 1H), 2.32-2.05 (m, 4H), 1.47 (d, J = 6.5 Hz, 3H). LCMS m/z 429.18 [M + H]⁺. |
| 702 | 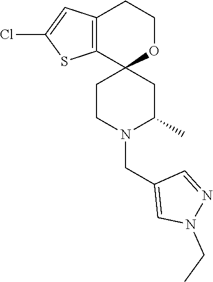 [SMILES: CCn1cc(CN2CC[C@]3(C[C@@H]2C)OCCc2cc(Cl)sc23)cn1] | 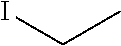 | 699¹ | ¹H NMR (400 MHz, DMSO-d6) δ 11.08 (s, 1H), 7.97 (s, 1H), 7.63 (s, 1H), 6.91 (s, 1H), 4.34 (d, J = 13.9 Hz, 1H), 4.16 (q, J = 7.2 Hz, 3H), 3.92-3.74 (m, 2H), 3.31-3.15 (m, 2H), 3.08 (d, J = 19.2 Hz, 1H), 3.00-2.90 (m, 1H), 2.17 (dd, J = 20.6, 9.2 Hz, 4H), 1.46 (d, J = 6.4 Hz, 3H), 1.36 (d, J = 7.3 Hz, 3H). LCMS m/z 366.34 [M + H]⁺. |
| 703 | 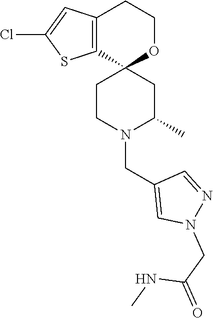 [SMILES: CNC(=O)Cn1cc(CN2CC[C@]3(C[C@@H]2C)OCCc2cc(Cl)sc23)cn1] | 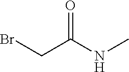 | 699 | ¹H NMR (400 MHz, DMSO-d6) δ 10.89 (s, 1H), 8.14 (d, J = 5.0 Hz, 1H), 7.95 (s, 1H), 7.65 (s, 1H), 6.91 (s, 1H), 4.81 (s, 2H), 4.38 (d, J = 13.9 Hz, 1H), 4.18 (d, J = 14.2 Hz, 1H), 3.85 (tt, J = 12.2, 5.9 Hz, 2H), 3.32-3.11 (m, 3H), 3.00 (s, 1H), 2.62 (d, J = 4.6 Hz, 3H), 2.19 (q, J = 14.3 Hz, 4H), 1.46 (d, J = 6.3 Hz, 3H). LCMS m/z 409.11 [M + H]⁺. |
| 704 | 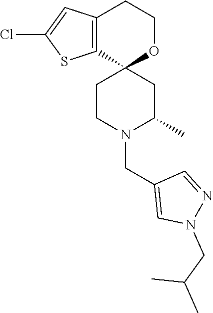 [SMILES: CC(C)Cn1cc(CN2CC[C@]3(C[C@@H]2C)OCCc2cc(Cl)sc23)cn1] | 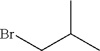 | 699¹ | ¹H NMR (400 MHz, DMSO-d6) δ 10.15 (s, 1H), 7.95 (s, 1H), 7.63 (s, 1H), 6.92 (s, 1H), 4.38 (d, J = 13.7 Hz, 1H), 4.22 (dd, J = 13.8, 5.3 Hz, 1H), 3.95 (d, J = 7.2 Hz, 2H), 3.82 (h, J = 6.3 Hz, 2H), 3.38-3.12 (m, 2H), 3.01 (d, J = 11.8 Hz, 1H), 2.55 (t, J = 5.3 Hz, 2H), 2.21 (dd, J = 25.4, 14.3 Hz, 1H), 2.15-2.03 (m, 1H), 1.97 (dd, J = 14.6, 12.0 Hz, 1H), 1.43 (d, J = 6.4 Hz, 2H), 0.83 (d, J = 6.7 Hz, 3H), −0.14 (s, 6H). LCMS m/z 394.33 [M + H]⁺. |
| 705 | 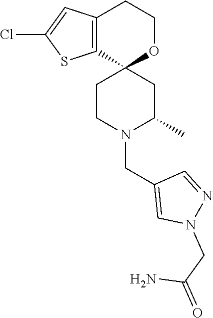 [SMILES: C[C@H]1C[C@@]2(CCN1Cc1cnn(CC(N)=O)c1)OCCc1cc(Cl)sc12] | 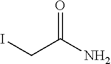 | 699 | LCMS m/z 394.92 [M + H]⁺. |
| 706 | 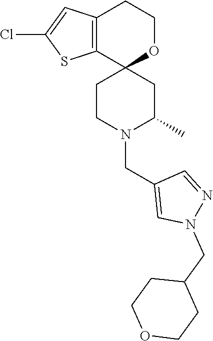 [SMILES: C[C@H]1C[C@@]2(CCN1Cc1cnn(CC3CCOCC3)c1)OCCc1cc(Cl)sc12] | 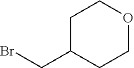 | 699¹ | ¹H NMR (400 MHz, DMSO-d6) δ 11.19 (s, 1H), 7.96 (s, 1H), 7.65 (s, 1H), 6.90 (s, 1H), 4.32 (d, J = 14.0 Hz, 1H), 4.18 (dd, J = 14.1, 5.1 Hz, 1H), 4.04 (d, J = 7.2 Hz, 2H), 3.81 (q, J = 8.7, 6.2 Hz, 4H), 3.28- 3.03 (m, 5H), 3.01-2.86 (m, 1H), 2.17 (q, J = 14.4 Hz, 4H), 2.05 (d, J = 6.8 Hz, 1H), 1.46 (d, J = 6.3 Hz, 3H), 1.33-1.17 (m, 5H). LCMS m/z 436.37 [M + H]⁺. |
| 707 | 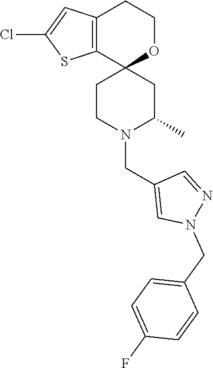 [SMILES: C[C@H]1C[C@@]2(CCN1Cc1cnn(Cc3ccc(F)cc3)c1)OCCc1cc(Cl)sc12] | 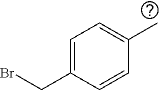 | 699¹ | ¹H NMR (400 MHz, DMSO-d6) δ 10.96 (s, 1H), 8.08 (s, 1H), 7.68 (s, 1H), 7.30 (dd, J = 8.5, 5.6 Hz, 2H), 7.19 (t, J = 8.8 Hz, 2H), 6.91 (s, 1H), 5.36 (s, 2H), 4.35 (d, J = 14.0 Hz, 1H), 4.27-4.10 (m, 1H), 3.84 (tt, J = 12.1, 6.7 Hz, 2H), 3.29-3.13 (m, 2H), 3.00 (s, 1H), 2.18 (q, J = 15.0 Hz, 4H), 1.45 (d, J = 6.3 Hz, 3H). LCMS m/z 446.3 [M + H]⁺. |

Footnotes:
¹The reaction stirred at room temperature for 10 min.
2. Purified with formic acid modifier. Isolated the product as formic acid salt

[symbol]

[symbol]

indicates data missing or illegible when filed

### Compound 708

### (2S)-3-[(2′S, 7R)-2-chloro-2′-methyl-spiro[4,5-dihydrothieno[2,3-c]pyran-7,4′-piperidine]-1′-yl]-2-hydroxy-propanamide

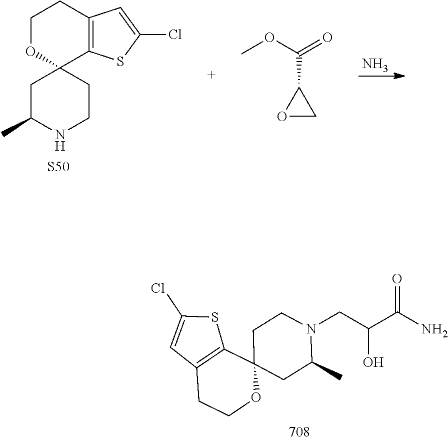

### Preparation of (2S)-3-[(2′S, 7R)-2-chloro-2′-methyl-spiro[4,5-dihydrothieno[2,3-c]pyran-7,4′-piperidine]-1′-yl]-2-hydroxy-propanamide (708)

(2′S,7R)-2-chloro-2′-methyl-spiro[4,5-dihydrothieno[2,3-c]pyran-7,4′-piperidine]S50 (99.1 mg, 0.3844 mmol) and methyl (2S)-oxirane-2-carboxylate (100 μL, 1.142 mmol) were added to a microwave vial and brought up in NH₃ (3.5 mL of 7 M, 24.50 mmol) and MeOH (99 μL). DIPEA (340 μL, 1.952 mmol) was added and reaction stirred at 120° C. for 4 h. An additional 70 μL oxirane and 800 μL NH₃/MeOH were added and the reaction was heated for 2 h at 120° C. An additional 50 μL oxirane and 500 μL NH₃/MeOH were added and the reaction was heated for an additional 2 h at 120° C. Reaction mixture was concentrated down via rotovap, then diluted in water/DCM and the organics were extracted (3×DCM, 3×EtOAc) and concentrated down via rotovap. Material was purified via normal phase chromatography (12 g HP silica gel, 0-100% 90:10 MeOH/DCM in DCM) to afford desired product 708 (2S)-3-[(2′S,7R)-2-chloro-2′-methyl-spiro[4,5-dihydrothieno[2,3-c]pyran-7,4′-piperidine]-1′-yl]-2-hydroxy-propanamide (10.2 mg, 7%). ¹H NMR (300 MHz, Chloroform-d) δ 7.34 (s, 1H), 6.58 (s, 1H), 5.66 (s, 1H), 4.03 (dd, J=7.8, 5.8 Hz, 1H), 3.95-3.76 (m, 2H), 3.18 (dd, J=13.3, 7.8 Hz, 1H), 2.89 (dtd, J=11.5, 6.3, 2.3 Hz, 1H), 2.82-2.72 (m, 2H), 2.62-2.49 (m, 3H), 2.02 (d, J=2.3 Hz, 1H), 1.97 (q, J=2.8 Hz, 1H), 1.77 (ddd, J=13.8, 10.5, 6.7 Hz, 1H), 1.52 (dd, J=14.0, 11.4 Hz, 1H), 1.10 (d, J=6.3 Hz, 3H). LCMS m/z 345.08 [M+1]⁺.

### Compound 709

### (2R)-3-[(2′S, 7R)-2-chloro-2′-methyl-spiro[4,5-dihydrothieno[2,3-c]pyran-7,4′-piperidine]-1′-yl]-2-hydroxy-propanamide

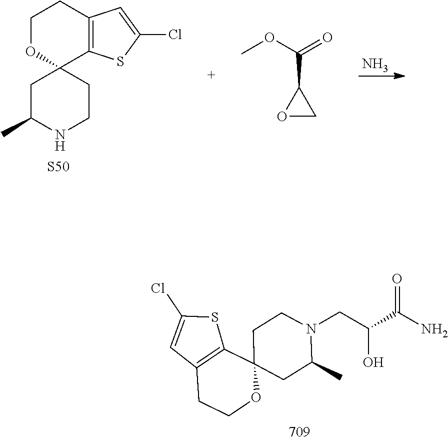

### Preparation of (2R)-3-[(2′S, 7R)-2-chloro-2′-methyl-spiro[4,5-dihydrothieno[2,3-c]pyran-7,4′-piperidine]-1′-yl]-2-hydroxy-propanamide (709)

(2′S,7R)-2-chloro-2′-methyl-spiro[4,5-dihydrothieno[2,3-c]pyran-7,4′-piperidine]S50 (107.5 mg, 0.4170 mmol) and methyl (2R)-oxirane-2-carboxylate (360 μL, 4.112 mmol) were added to a microwave vial and brought up in NH₃ (4.7 mL of 7 M, 32.90 mmol) in MeOH (4.7 mL). DIPEA (750 μL, 4.306 mmol) was added and reaction stirred at 120° C. for 5 h. An additional 200 μL oxirane and 500 μL NH₃ were added and the reaction was stirred an additional 4 h at 120° C. Material was concentrated down via rotovap and rinsed with MeOH/DCM (2×). Remaining solid was insoluble in DCM to load onto column, so citric acid was added to acidify and the organics were extracted with EtOAc (3×). Organic layer was dried down via rotovap and material was purified via normal phase chromatography (40 g silica gel 0-20% MeOH/DCM) afforded product (2R)-3-[(2′S,7R)-2-chloro-2′-methyl-spiro[4,5-dihydrothieno[2,3-c]pyran-7,4′-piperidine]-1′-yl]-2-hydroxy-propanamide (36.9 mg, 22%). ¹H NMR (300 MHz, Chloroform-d) δ 6.93 (d, J=4.4 Hz, 1H), 6.56 (s, 1H), 5.88 (s, 1H), 4.07 (d, J=7.1 Hz, 3H), 3.95-3.77 (m, 2H), 2.92 (dd, J=12.9, 9.9 Hz, 1H), 2.85-2.75 (m, 2H), 2.70-2.42 (m, 4H), 1.98 (p, J=2.7 Hz, 1H), 1.73 (td, J=13.4, 4.6 Hz, 1H), 1.51 (dd, J=14.0, 11.3 Hz, 1H), 1.04 (d, J=6.2 Hz, 3H). LCMS m/z 345.03 [M+1]⁺.

### Compound 710

### (2S)-3-[(2′S, 7R)-2-chloro-2′-methyl-spiro[4,5-dihydrothieno[2,3-c]pyran-7,4′-piperidine]-1′-yl]-2-hydroxy-N-methyl-propanamide

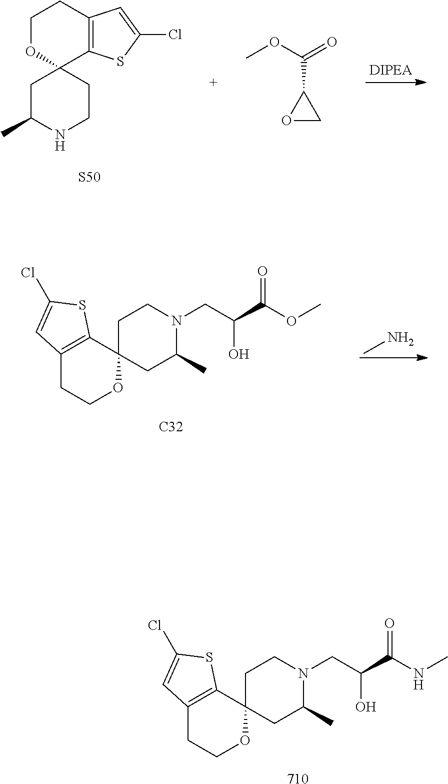

### Step 1: Synthesis of methyl (2S)-3-[(2′S, 7R)-2-chloro-2′-methyl-spiro[4,5-dihydrothieno[2,3-c]pyran-7,4′-piperidine]-1′-yl]-2-hydroxy-propanoate (C32)

(2′S,7R)-2-chloro-2′-methyl-spiro[4,5-dihydrothieno[2,3-c]pyran-7,4′-piperidine]S50 (176 mg, 0.6828 mmol) was brought up in MeOH (3.5 mL) and added to a microwave vial. methyl (2S)-oxirane-2-carboxylate (250 μL, 2.855 mmol) and DIPEA (600 μL, 3.445 mmol) were added, and the reaction was heated at 90° C. for 1 h. Reaction mixture was concentrated via rotovap, and purified via normal phase chromatography (24 g silica gel 0-80% EtOAC/Heptanes) to afford methyl (2S)-3-[(2′S,7R)-2-chloro-2′-methyl-spiro[4,5-dihydrothieno[2,3-c]pyran-7,4′-piperidine]-1′-yl]-2-hydroxy-propanoate C32 (147.5 mg, 57%). LCMS m/z 360.02 [M+1]⁺.

### Step 2: Synthesis of (2S)-3-[(2′S, 7R)-2-chloro-2′-methyl-spiro[4,5-dihydrothieno[2,3-c]pyran-7,4′-piperidine]-1′-yl]-2-hydroxy-N-methyl-propanamidemethyl (710)

(2S)-3-[(2′S,7R)-2-chloro-2′-methyl-spiro[4,5-dihydrothieno[2,3-c]pyran-7,4′-piperidine]-1′-yl]-2-hydroxy-propanoate C32 (35.7 mg, 0.09920 mmol) was brought up in dioxane (700 μL) in a microwave flask. Methyl amine (100 μL of 40% w/v, 1.288 mmol) and water (17 μL, 0.9436 mmol) were added and the reaction was heated to 80° C. for 3 h. Purification by reversed-phase HPLC. Method: C18 Waters Sunfire column (30×150 mm, 5 micron). afforded the product (2S)-3-[(2′S,7R)-2-chloro-2′-methyl-spiro[4,5-dihydrothieno[2,3-c]pyran-7,4′-piperidine]-1′-yl]-2-hydroxy-N-methyl-propanamide 710 (22.4 mg, 62%). 1H NMR (300 MHz, Chloroform-d) δ 10.65 (s, 1H), 7.46 (s, 1H), 6.61 (s, 1H), 4.70 (dd, J=9.7, 3.1 Hz, 1H), 4.04 (d, J=13.7 Hz, 1H), 3.89 (p, J=5.8 Hz, 3H), 3.66 (d, J=12.0 Hz, 1H), 3.53 (s, 1H), 3.32 (q, J=11.6, 10.9 Hz, 1H), 2.87 (d, J=4.9 Hz, 3H), 2.63 (q, J=5.2 Hz, 3H), 2.32 (q, J=13.7, 12.7 Hz, 1H), 2.17 (d, J=15.6 Hz, 2H), 1.49 (dd, J=6.5, 2.2 Hz, 4H). LCMS m/z 359.42 [M+1]⁺.

### Compound 711

### (2S)-3-[(2′S, 7R)-2-chloro-2′-methyl-spiro[4,5-dihydrothieno[2,3-c]pyran-7,4′-piperidine]-1′-yl]-2-hydroxy-N,N-dimethyl-propanamide

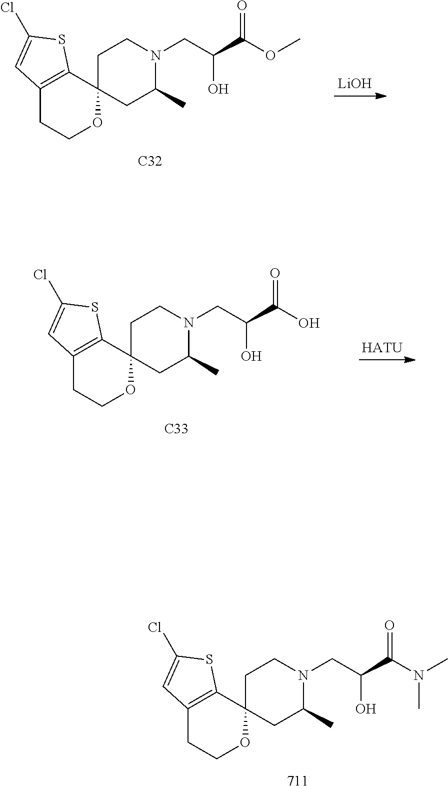

### Step 1: Synthesis of (2S)-3-[(2′S, 7R)-2-chloro-2′-methyl-spiro[4,5-dihydrothieno[2,3-c]pyran-7,4′-piperidine]-1′-yl]-2-hydroxy-propanoic acid (C33)

A solution of methyl (2S)-3-[(2′S,7R)-2-chloro-2′-methyl-spiro[4,5-dihydrothieno[2,3-c]pyran-7,4′-piperidine]-1′-yl]-2-hydroxy-propanoate C32 (107 mg, 0.2973 mmol) and LiOH (31.6 mg, 1.320 mmol) in MeOH (1.1 mL) and THF (1 mL), and stirred at rt for 2 h. Reaction mixture was concentrated down via rotovap and crude material was used without purification. (2S)-3-[(2′S,7R)-2-chloro-2′-methyl-spiro[4,5-dihydrothieno[2,3-c]pyran-7,4′-piperidine]-1′-yl]-2-hydroxy-propanoic acid (Lithium salt) C33 (147 mg, 98%) LCMS m/z 344 [M+1]⁺.

### Step 2: Synthesis of (2S)-3-[(2′S, 7R)-2-chloro-2′-methyl-spiro[4,5-dihydrothieno[2,3-c]pyran-7,4′-piperidine]-1′-yl]-2-hydroxy-N,N-dimethyl-propanamide (711)

(2S)-3-[(2′S,7R)-2-chloro-2′-methyl-spiro[4,5-dihydrothieno[2,3-c]pyran-7,4′-piperidine]-1′-yl]-2-hydroxy-propanoic acid C33 (Lithium salt) (147 mg, 0.2917 mmol), N-methylmethanamine (500 μL of 2 M, 1.000 mmol), and HATU (379.5 mg, 0.9981 mmol) were brought up in DMF (2 mL). DIPEA (200 μL, 1.148 mmol) was added and the reaction was allowed to stir 2 h. An additional 500 μL DMF was added to rinse down sides of flask. An additional 332 mg HATU in 500 μL DMF and 300 μL dimethylamine were added and reaction was stirred overnight. Reaction mixture was concentrated via rotovap. An excess of water was added, and organics were extracted with DCM and concentrated via rotovap. Purification by silica gel chromatography (0-20% MeOH/DCM). followed by silica gel chromatography (0-15% MeOH/DCM) afforded (2S)-3-[(2′S,7R)-2-chloro-2′-methyl-spiro[4,5-dihydrothieno[2,3-c]pyran-7,4′-piperidine]-1′-yl]-2-hydroxy-N,N-dimethyl-propanamide 711 (7.6 mg, 6%). ¹H NMR (300 MHz, Chloroform-d) δ 6.60 (s, 1H), 4.69 (dd, J=7.4, 4.0 Hz, 1H), 4.14 (q, J=7.1 Hz, 1H), 4.03-3.79 (m, 2H), 3.33-2.94 (m, 11H), 2.80-2.50 (m, 3H), 2.24-2.08 (m, 1H), 2.05-1.88 (m, 2H), 1.71 (dd, J=14.1, 11.6 Hz, 1H), 1.33-1.07 (m, 4H). LCMS m/z 373.12 [M+1]⁺.

### Preparation S53

### [(2′S, 7R)-2-chloro-2′-methyl-spiro[4,5-dihydrothieno[2,3-c]pyran-7,4′-piperidine]-3-yl]methanol (S53)

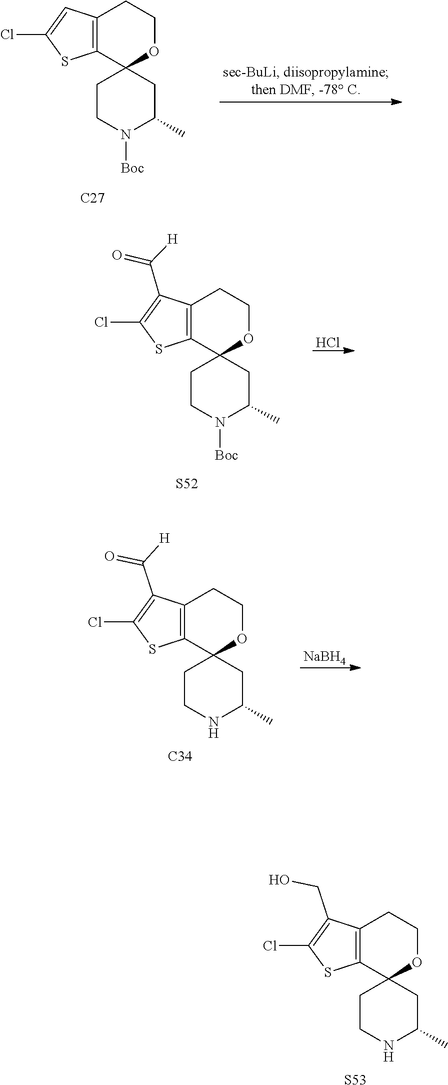

### Step 1. Synthesis of tert-butyl (2′S, 7R)-2-chloro-3-formyl-2′-methyl-spiro[4,5-dihydrothieno[2,3-c]pyran-7,4′-piperidine]-1′-carboxylate (S52) —Preparation of LDA Solution

A solution of diisopropylamine (3 mL, 21.41 mmol) in Et₂O (3 mL) in an oven dried flask was purged with nitrogen and cooled to −20° C. At this time, sec-butyllithium (15 mL of 1.333 M, 19.99 mmol) was added dropwise. The reaction mixture was stirred at this temperature for 30 min.

A solution of tert-butyl (2′S,7R)-2-chloro-2′-methyl-spiro[4,5-dihydrothieno[2,3-c]pyran-7,4′-piperidine]-1′-carboxylate C27 (6.3 g, 17.49 mmol) in THF (100 mL) in an oven dried flask was purged with nitrogen and cooled to −78° C. At this time, the mixture of LDA from the previous step was transferred over the course of 2 min. The mixture was stirred at this temperature for 5 min. At this time, DMF (5 mL, 64.57 mmol) was added. After 5 min, to this mixture was added sat. aqueous ammonium chloride (25 mL), and the mixture was warmed to room temperature. The mixture was then diluted with Et₂O (200 mL), followed by water (200 mL). The organic layer was washed with additional water (2×200 mL) and brine (200 mL). The organic layer was dried with sodium sulfate, filtered, and concentrated in vacuo. The mixture was purified by silica gel chromatography (Gradient: 0-25% EtOAc in heptane) to yield tert-butyl (2′S,7R)-2-chloro-3-formyl-2′-methyl-spiro[4,5-dihydrothieno[2,3-c]pyran-7,4′-piperidine]-1′-carboxylate (5.0 g, 73%). ¹H NMR (400 MHz, Chloroform-d) δ 10.09 (s, 1H), 4.01 (tt, J=11.6, 6.5 Hz, 1H), 3.95-3.80 (m, 2H), 3.74 (dt, J=14.0, 5.5 Hz, 1H), 3.36 (ddd, J=13.9, 8.6, 5.3 Hz, 1H), 2.99-2.84 (m, 2H), 2.21 (dddd, J=14.6, 8.3, 5.9, 1.9 Hz, 1H), 2.12 (ddd, J=14.1, 5.1, 2.0 Hz, 1H), 1.76 (dt, J=14.5, 5.2 Hz, 1H), 1.67 (dd, J=14.2, 10.9 Hz, 1H), 1.50 (s, 9H), 1.29 (d, J=6.5 Hz, 3H). LCMS m/z 385.96 [M+H]⁺.

### Step 2. Synthesis of (2′S, 7R)-2-chloro-2′-methyl-spiro[4,5-dihydrothieno[2,3-c]pyran-7,4′-piperidine]-3-carbaldehyde (C34)

A mixture of tert-butyl (2′S,7R)-2-chloro-3-formyl-2′-methyl-spiro[4,5-dihydrothieno[2,3-c]pyran-7,4′-piperidine]-1′-carboxylate S52 (3.0 g, 7.649 mmol) in a dioxane solution of HCl (7.5 mL of 4 M, 30.00 mmol) was stirred at room temperature. After 5 min, Et₂O (15 mL) was added and the suspension was stirred at room temperature. After 30 min, the mixture was filtered, rinsed with Et₂O and concentrated to yield (2′S,7R)-2-chloro-2′-methyl-spiro[4,5-dihydrothieno[2,3-c]pyran-7,4′-piperidine]-3-carbaldehyde (Hydrochloride salt) (2.369 g, 96%). LCMS m/z 286.05 [M+H]⁺.

### Step 3. Synthesis of [(2′S, 7R)-2-chloro-2′-methyl-spiro[4,5-dihydrothieno[2,3-c]pyran-7,4′-piperidine]-3-yl]methanol (S53)

To (2′S,7R)-2-chloro-2′-methyl-spiro[4,5-dihydrothieno[2,3-c]pyran-7,4′-piperidine]-3-carbaldehyde (Hydrochloride salt) C34 (2.369 g, 7.35 mmol) dissolved in water (40 mL) was added NaBH₄ (100 mg, 2.643 mmol). Aqueous HCl (7.5 mL of 1 M, 7.500 mmol) was then added till pH=1. After stirring for 5 min, the mixture pH was then adjusted with NaOH (2.5 mL of 6 M, 15.00 mmol) to about pH=12. The mixture was extracted with DCM (2×25 mL), dried with sodium sulfate, filtered and concentrated to provide [(2′S,7R)-2-chloro-2′-methyl-spiro[4,5-dihydrothieno[2,3-c]pyran-7,4′-piperidine]-3-yl]methanol (1.88 g, 82%) as a white amorphous solid. ¹H NMR (400 MHz, DMSO-d₆) δ 5.03 (t, J=5.4 Hz, 1H), 4.31 (d, J=5.2 Hz, 2H), 3.86 (td, J=5.7, 3.0 Hz, 2H), 2.95-2.78 (m, 2H), 2.78-2.68 (m, 1H), 2.59 (t, J=5.5 Hz, 2H), 1.93-1.80 (m, 2H), 1.49 (td, J=13.0, 4.8 Hz, 1H), 1.18 (dd, J=13.4, 11.2 Hz, 1H), 0.94 (d, J=6.4 Hz, 3H). LCMS m/z 288.08 [M+H]⁺.

### Compound 712

### 2-chloro-6-[[(2′S, 7R)-2-chloro-3-(hydroxymethyl)-2′-methyl-spiro[4,5-dihydrothieno[2,3-c]pyran-7,4′-piperidine]-1′-yl]methyl]phenol (712)

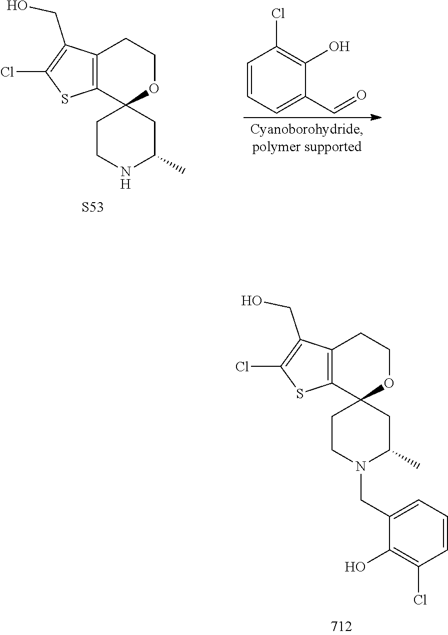 [SMILES: C[C@H]1C[C@@]2(CCN1)OCCc1c2sc(Cl)c1CO ; O=Cc1cccc(Cl)c1O ; "S53" ; C[C@H]1C[C@@]2(CCN1Cc1cccc(Cl)c1O)OCCc1c2sc(Cl)c1CO]

Standard Method #A: Reductive Amination with Polymer Supported Cyanoborohydride

To a 1-dram vial was added cyanoborohydride, polymer supported (75 mg of 2 mmol/g, 0.15 mmol), followed by a solution 3-chloro-2-hydroxy-benzaldehyde (15.7 mg, 0.1 mmol) in DMF (400 uL) and a solution of [(2′S,7R)-2-chloro-2′-methyl-spiro[4,5-dihydrothieno[2,3-c]pyran-7,4′-piperidine]-3-yl]methanol S53 (16 mg, 0.056 mmol) in DMF (400 uL). The vial was sealed and heated at 85° C. overnight. Then the vial was cooled to room temperature. The resulting suspension was filtered through a polypropylene filter plate. 0.1 wt % TFA (400 uL) in water was added to the reaction vial, which was stirred for several min to rinse the beads. DMSO (200 uL) was added to the resulting filtrate. Purification by reversed-phase HPLC. Method: C18 Waters Sunfire column (30×150 mm, 5 micron). Gradient: MeCN in H₂O with 0.1% trifluoroacetic acid. 2-chloro-6-[[(2′S,7R)-2-chloro-3-(hydroxymethyl)-2′-methyl-spiro[4,5-dihydrothieno[2,3-c]pyran-7,4′-piperidine]-1′-yl]methyl]phenol (trifluoroacetic acid salt) (2.8 mg, 9.2%). LCMS m/z 428.29 [M+H]⁺.

### Compound 713

### (2′S, 7R)-2-chloro-1′-[(2-fluorophenyl)methyl]-2′-methyl-spiro[4,5-dihydrothieno[2,3-c]pyran-7,4′-piperidine](713)

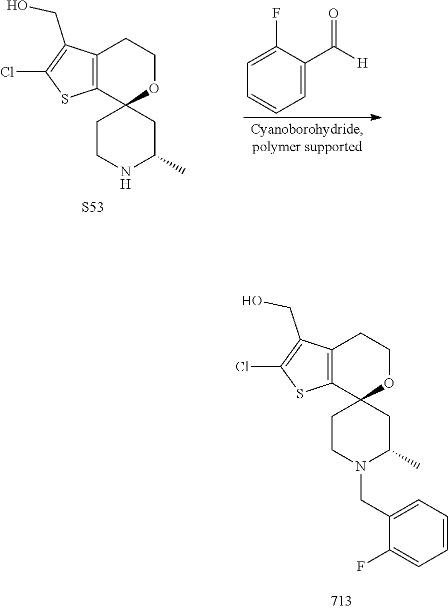 [SMILES: C[C@H]1C[C@@]2(CCN1)OCCc1c2sc(Cl)c1CO ; O=Cc1ccccc1F ; "S53" ; C[C@H]1C[C@@]2(CCN1Cc1ccccc1F)OCCc1c2sc(Cl)c1CO]

Standard Method #B: Reductive Amination with Polymer Supported Cyanoborohydride and Acetic Acid

A stock solution of [(2′S,7R)-2-chloro-2′-methyl-spiro[4,5-dihydrothieno[2,3-c]pyran-7,4′-piperidine]-3-yl]methanol S53 (20 mg, 0.068 mmol) and AcOH (2 μL) in DMF (0.7 mL) was added to a mixture of 2-fluorobenzaldehyde (25 μL, 0.237 mmol) in DMF (0.7 mL). To this mixture was added cyanoborohydride, polymer supported (75 mg of 2 mmol/g, 0.15 mmol), and the mixture was heated to 85° C. After stirring overnight, the mixtures were cooled to room temperature, filtered and the polymer was rinsed with additional DMF. Purification by reversed-phase HPLC. Method: C18 Waters Sunfire column (30×150 mm, 5 micron). Gradient: MeCN in H₂O with 0.1% trifluoroacetic acid. (2′S,7R)-2-chloro-1′-[(2-fluorophenyl)methyl]-2′-methyl-spiro[4,5-dihydrothieno[2,3-c]pyran-7,4′-piperidine](trifluoroacetic acid salt) (19.3 mg, 56%). ¹H NMR (400 MHz, Chloroform-d) δ 7.74-7.65 (m, 1H), 7.48 (q, J=6.3 Hz, 1H), 7.31 (s, 1H), 7.21-7.14 (m, 1H), 4.63 (d, J=13.4 Hz, 1H), 4.57 (s, 2H), 4.13 (d, J=13.2 Hz, 1H), 3.91 (dq, J=21.8, 5.9 Hz, 2H), 3.52 (s, 1H), 3.22 (d, J=12.1 Hz, 1H), 3.05 (t, J=12.6 Hz, 1H), 2.71 (q, J=5.5, 5.1 Hz, 2H), 2.53-2.37 (m, 2H), 2.22-2.03 (m, 2H), 1.68 (d, J=6.5 Hz, 3H). LCMS m/z 396.32 [M+H]⁺.

### Compounds 714-758

Compounds 714-758 (see Table 31) were prepared in a single step from intermediate S53 using method as for preparation of 712 or preparation of 713. Aldehydes were commercially available or described previously. Any modifications to methods are noted in Table 31 and accompanying footnotes.

TABLE 31
Structure and physicochemical data for compounds 714-758

| | | Aldehyde | | ¹H NMR; LCMS m/z |
|---|---|---|---|---|
| Cmpd | Structure | Reagent | Method | [M + H]⁺ |
| 714 | 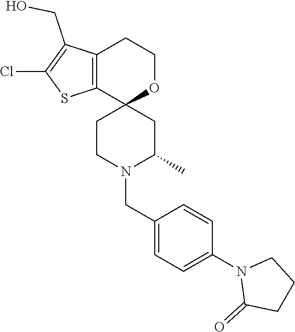 [SMILES: C[C@H]1C[C@@]2(CCN1Cc1ccc(N3CCCC3=O)cc1)OCCc1c2sc(Cl)c1CO] | 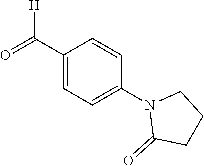 [SMILES: O=Cc1ccc(N2CCCC2=O)cc1] | 712 | LCMS m/z 461.38 [M + H]⁺ |
| 715 | 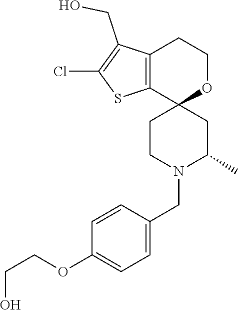 [SMILES: C[C@H]1C[C@@]2(CCN1Cc1ccc(OCCO)cc1)OCCc1c2sc(Cl)c1CO] | 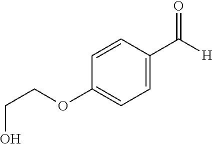 [SMILES: O=Cc1ccc(OCCO)cc1] | 712 | LCMS m/z 438.31 [M + H]⁺ |
| 716 | 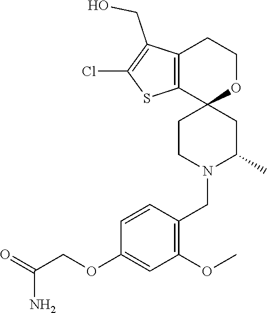 [SMILES: COc1cc(OCC(N)=O)ccc1CN1CC[C@]2(C[C@@H]1C)OCCc1c2sc(Cl)c1CO] | 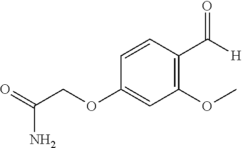 | 712 | LCMS m/z 481.34 [M + H]⁺ |
| 717 | 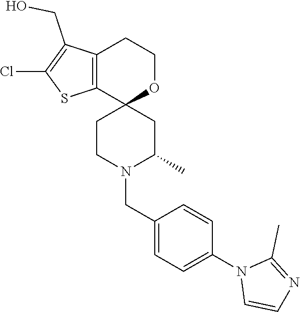 [SMILES: Cc1nccn1-c1ccc(CN2CC[C@]3(C[C@@H]2C)OCCc2c3sc(Cl)c2CO)cc1] | 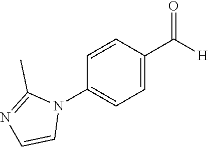 | 712 | LCMS m/z 458.36 [M + H]⁺ |
| 718 | 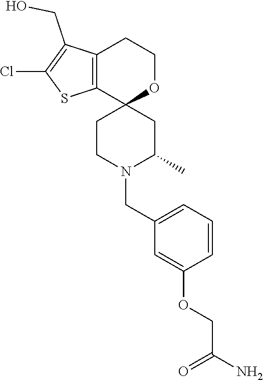 [SMILES: C[C@H]1C[C@@]2(CCN1Cc1cccc(OCC(N)=O)c1)OCCc1c2sc(Cl)c1CO] | 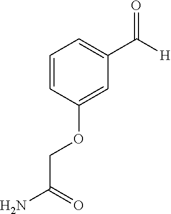 [SMILES: NC(=O)COc1cccc(C=O)c1] | 712 | LCMS m/z 451.31 [M + H]⁺ |
| 719 | 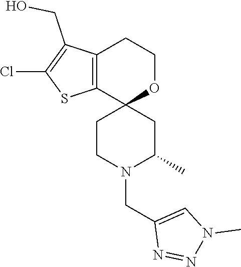 [SMILES: C[C@H]1C[C@@]2(CCN1Cc1cn(C)nn1)OCCc1c2sc(Cl)c1CO] | 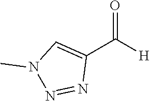 | 712 | ¹H NMR (400 MHZ, DMSO-d₆) δ 10.03 (s, 1H), 8.31 (s, 1H), 5.09 (s, 1H), 4.62 (d, J = 14.2 Hz, 1H), 4.51 (d, J = 14.4 Hz, 1H), 4.32 (s, 2H), 4.11 (s, 3H), 3.87 (q, J = 6.0 Hz, 2H), 3.33 (d, J = 12.6 Hz, 2H), 3.17 (d, J = 11.9 Hz, 1H), 2.61 (t, J = 5.5 Hz, 2H), 2.38- 2.08 (m, 2H), 2.05- 1.82 (m, 2H), 1.45 (d, J = 6.3 Hz, 3H); LCMS m/z 383.36 [M + H]⁺ |
| 720 | 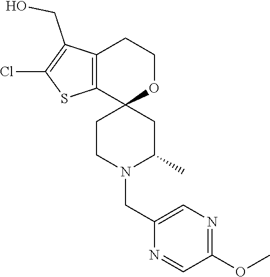 [SMILES: COc1cnc(CN2CC[C@]3(C[C@@H]2C)OCCc2c3sc(Cl)c2CO)cn1] | 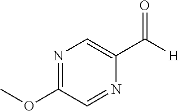 | 712 | LCMS m/z 410.32 [M + H]⁺ |
| 721 | 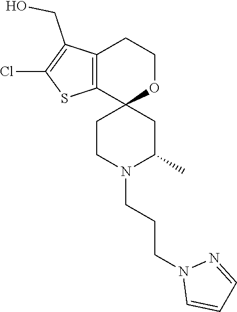 [SMILES: C[C@H]1C[C@@]2(CCN1CCCn1cccn1)OCCc1c2sc(Cl)c1CO] | 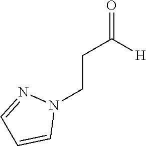 | 712 | ¹H NMR (400 MHZ, DMSO-d₆) δ 9.54 (s, 1H), 7.78 (d, J = 2.3 Hz, 1H), 7.49 (d, J = 1.8 Hz, 1H), 6.28 (t, J = 2.1 Hz, 1H), 5.03 (d, J = 6.1 Hz, 1H), 4.33 (s, 2H), 4.23 (t, J = 6.7 Hz, 2H), 3.92 (h, J = 6.4 Hz, 2H), 3.41 (s, 2H), 3.34-3.13 (m, 2H), 3.03 (t, J = 14.0 Hz, 1H), 2.64 (t, J = 5.5 Hz, 2H), 2.31- 2.07 (m, 4H), 1.99 (t, J = 12.1 Hz, 1H), 1.91- 1.82 (m, 1H), 1.20 (d, J = 6.3 Hz, 3H); LCMS m/z 396.36 [M + H]⁺ |
| 722 | 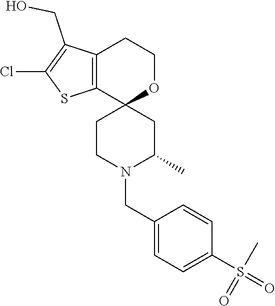 [SMILES: C[C@H]1C[C@@]2(CCN1Cc1ccc(S(C)(=O)=O)cc1)OCCc1c2sc(Cl)c1CO] | 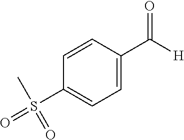 | 712 | LCMS m/z 456.28 [M + H]⁺ |
| 723 | 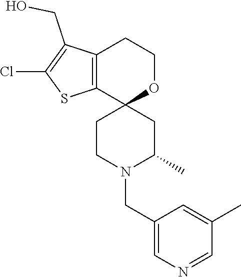 [SMILES: Cc1cncc(CN2CC[C@]3(C[C@@H]2C)OCCc2c3sc(Cl)c2CO)c1] | 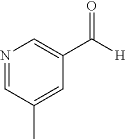 | 712 | LCMS m/z 393.34 [M + H]⁺ |
| 724 | 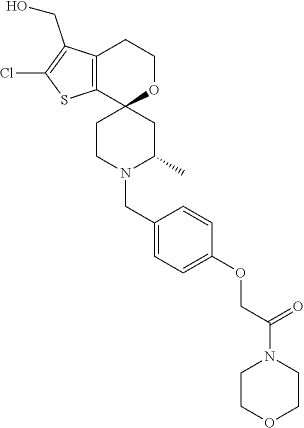 [SMILES: C[C@H]1C[C@@]2(CCN1Cc1ccc(OCC(=O)N3CCOCC3)cc1)OCCc1c2sc(Cl)c1CO] | 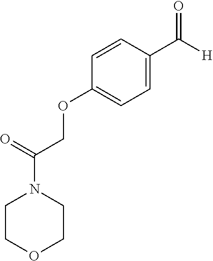 [SMILES: O=Cc1ccc(OCC(=O)N2CCOCC2)cc1] | 712 | LCMS m/z 521.43 [M + H]⁺ |
| 725 | 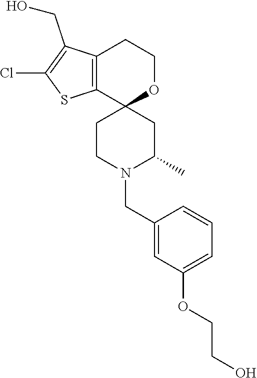 [SMILES: C[C@H]1C[C@@]2(CCN1Cc1cccc(OCCO)c1)OCCc1c2sc(Cl)c1CO] | 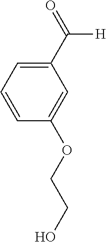 [SMILES: O=Cc1cccc(OCCO)c1] | 712 | LCMS m/z 438.31 [M + H]⁺ |
| 726 | 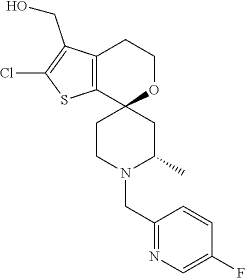 [SMILES: C[C@H]1C[C@@]2(CCN1Cc1ccc(F)cn1)OCCc1c2sc(Cl)c1CO] | 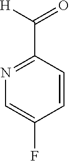 | 712 | LCMS m/z 397.31 [M + H]⁺ |
| 727 | 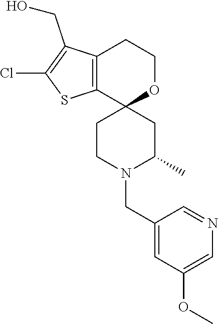 [SMILES: COc1cncc(CN2CC[C@]3(C[C@@H]2C)OCCc2c3sc(Cl)c2CO)c1] | 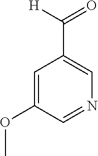 | 712 | LCMS m/z 409.32 [M + H]⁺ |
| 728 | 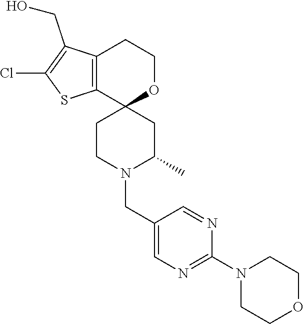 [SMILES: C[C@H]1C[C@@]2(CCN1Cc1cnc(N3CCOCC3)nc1)OCCc1c2sc(Cl)c1CO] | 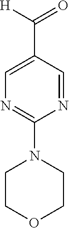 [SMILES: O=Cc1cnc(N2CCOCC2)nc1] | 712 | LCMS m/z 465.4 [M + H]⁺ |
| 729 | 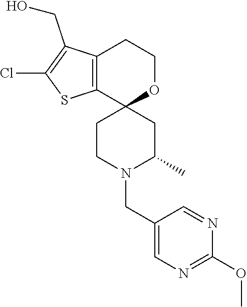 [SMILES: COc1ncc(CN2CC[C@]3(C[C@@H]2C)OCCc2c3sc(Cl)c2CO)cn1] | 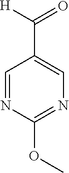 | 712 | LCMS m/z 410.32 [M + H]⁺ |
| 730 | 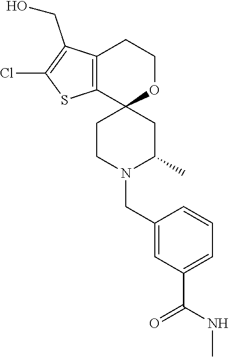 [SMILES: CNC(=O)c1cccc(CN2CC[C@]3(C[C@@H]2C)OCCc2c3sc(Cl)c2CO)c1] | 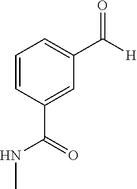 | 712 | ¹H NMR (400 MHZ, DMSO-d₆) δ 9.62 (s, 1H), 8.56 (d, J = 4.8 Hz, 1H), 8.00 (s, 1H), 7.91 (d, J = 7.7 Hz, 1H), 7.67 (d, J = 7.6 Hz, 1H), 7.58 (t, J = 7.7 Hz, 1H), 4.81 (d, J = 12.8 Hz, 1H), 4.32 (s, 2H), 4.20 (dd, J = 13.2, 8.1 Hz, 1H), 3.91 (dp, J = 23.3, 6.0 Hz, 2H), 3.57 (s, 1H), 3.04- 2.84 (m, 2H), 2.81 (d, J = 4.4 Hz, 3H), 2.63 (s, 3H), 2.29 (d, J = 15.0 Hz, 1H), 2.15 (d, J = 15.2 Hz, 1H), 1.90 (dt, J = 32.3, 12.3 Hz, 2H), 1.50 (d, J = 6.2 Hz, 3H); LCMS m/z 435.37 [M + H]⁺ |
| 731 | 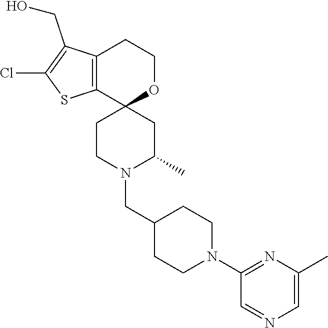 [SMILES: Cc1cncc(N2CCC(CN3CC[C@]4(C[C@@H]3C)OCCc3c4sc(Cl)c3CO)CC2)n1] | 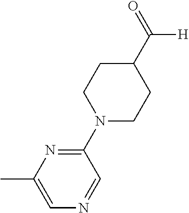 | 712 | ¹H NMR (400 MHZ, DMSO-d₆) δ 8.85 (s, 1H), 8.14 (s, 1H), 7.70 (s, 1H), 4.39 (s, 2H), 4.34 (s, 2H), 3.94 (s, 3H), 3.57 (d, J = 12.3 Hz, 1H), 3.46 (s, 1H), 3.24 (d, J = 55.0 Hz, 2H), 2.90 (dt, J = 25.8, 12.9 Hz, 4H), 2.68- 2.62 (m, 2H), 2.29 (s, 3H), 2.26-1.84 (m, 6H), 1.70 (d, J = 12.8 Hz, 1H), 1.30 (d, J = 6.4 Hz, 3H), 1.24 (d, J = 7.4 Hz, 1H); LCMS m/z 477.45 [M + H]⁺ |
| 732 | 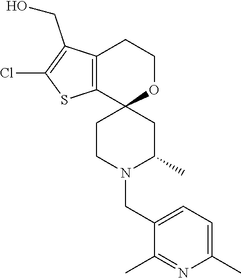 [SMILES: Cc1ccc(CN2CC[C@]3(C[C@@H]2C)OCCc2c3sc(Cl)c2CO)c(C)n1] | 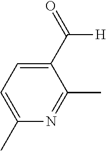 | 712 | LCMS m/z 407.38 [M + H]⁺ |
| 733 | 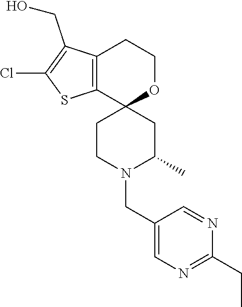 [SMILES: CCc1ncc(CN2CC[C@]3(C[C@@H]2C)OCCc2c3sc(Cl)c2CO)cn1] | 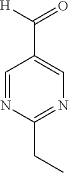 | 712 | LCMS m/z 408.37 [M + H]⁺ |
| 734 | 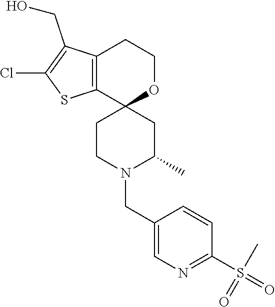 [SMILES: C[C@H]1C[C@@]2(CCN1Cc1ccc(S(C)(=O)=O)nc1)OCCc1c2sc(Cl)c1CO] | 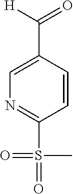 | 712 | LCMS m/z 457.28 [M + H]⁺ |
| 735 | 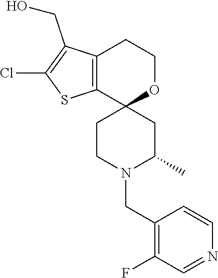 [SMILES: C[C@H]1C[C@@]2(CCN1Cc1ccncc1F)OCCc1c2sc(Cl)c1CO] | 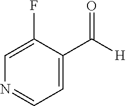 | 712 | LCMS m/z 397.31 [M + H]⁺ |
| 736 | 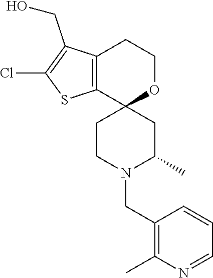 [SMILES: Cc1ncccc1CN1CC[C@]2(C[C@@H]1C)OCCc1c2sc(Cl)c1CO] | 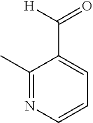 | 712 | LCMS m/z 393.34 [M + H]⁺ |
| 737 | 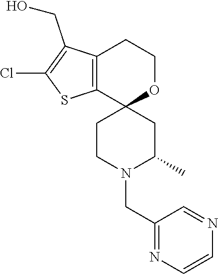 [SMILES: C[C@H]1C[C@@]2(CCN1Cc1cnccn1)OCCc1c2sc(Cl)c1CO] | 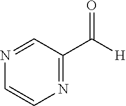 | 712 | LCMS m/z 380.33 [M + H]⁺ |
| 738 | 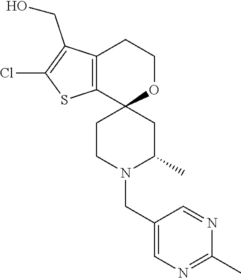 [SMILES: Cc1ncc(CN2CC[C@]3(C[C@@H]2C)OCCc2c3sc(Cl)c2CO)cn1] | 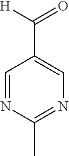 | 712 | LCMS m/z 394.33 [M + H]⁺ |
| 739 | 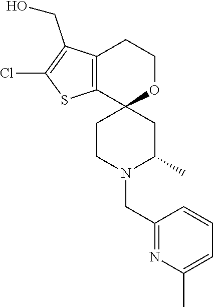 [SMILES: Cc1cccc(CN2CC[C@]3(C[C@@H]2C)OCCc2c3sc(Cl)c2CO)n1] | 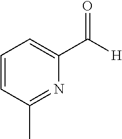 | 712 | LCMS m/z 393.34 [M + H]⁺ |
| 740 | 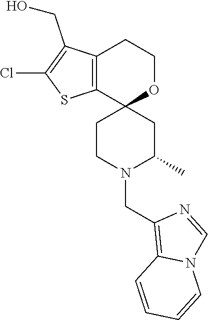 [SMILES: C[C@H]1C[C@@]2(CCN1Cc1ncn3ccccc13)OCCc1c2sc(Cl)c1CO] | 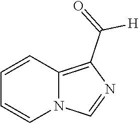 [SMILES: O=Cc1ncn2ccccc12] | 712 | ¹H NMR (400 MHZ, DMSO-d₆) δ 9.75 (s, 1H), 8.42 (d, J = 7.1 Hz, 1H), 7.85 (d, J = 9.3 Hz, 1H), 6.99 (d, J = 8.9 Hz, 1H), 6.79 (t, J = 6.7 Hz, 1H), 4.79 (d, J = 14.1 Hz, 1H), 4.63 (d, J = 14.1 Hz, 1H), 3.92 (d, J = 4.9 Hz, 1H), 3.85 (d, J = 5.3 Hz, 1H), 3.51 (s, 1H), 3.37 (s, 1H), 3.26 (d, J = 21.4 Hz, 1H), 3.12 (s, 1H), 2.64 (d, J = 6.7 Hz, 2H), 2.59 (s, 2H), 2.25 (d, J = 15.2 Hz, 1H), 2.16 (d, J = 15.0 Hz, 2H), 1.93 (d, J = |
| | | | | 14.8 Hz, 2H), 1.55 |
| | | | | (d, J = 6.3 Hz, 3H); |
| | | | | LCMS m/z 418.35 |
| | | | | [M + H]⁺ |
| 741 | 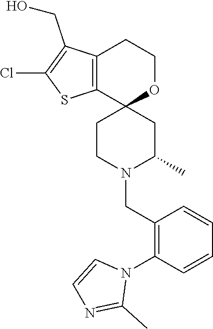 [SMILES: Cc1nccn1-c1ccccc1CN1CC[C@]2(C[C@@H]1C)OCCc1c2sc(Cl)c1CO] | 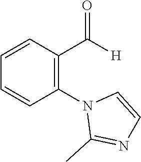 | 712 | LCMS m/z 458.4 [M + H]⁺ |
| 742 | 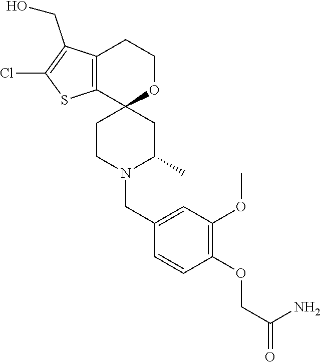 [SMILES: COc1cc(CN2CC[C@]3(C[C@@H]2C)OCCc2c3sc(Cl)c2CO)ccc1OCC(N)=O] | 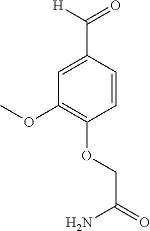 | 712 | LCMS m/z 481.34 [M + H]⁺ |
| 743 | 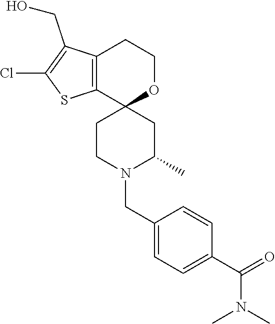 [SMILES: C[C@H]1C[C@@]2(CCN1Cc1ccc(C(=O)N(C)C)cc1)OCCc1c2sc(Cl)c1CO] | 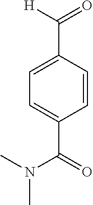 | 712 | LCMS m/z 449.37 [M + H]⁺ |
| 744 | 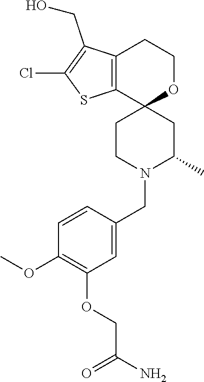 | 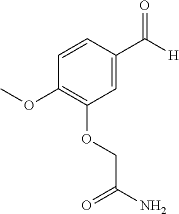 | 712 | LCMS m/z 481.34 [M + H]⁺ |
| 745 | 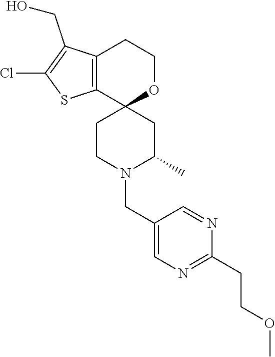 [SMILES: COCCc1ncc(CN2CC[C@]3(C[C@@H]2C)OCCc2c3sc(Cl)c2CO)cn1] | 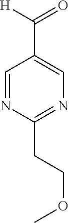 | 712 | LCMS m/z 438.35 [M + H]⁺ |
| 746 | 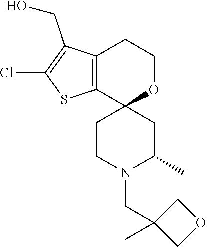 [SMILES: C[C@H]1C[C@@]2(CCN1CC1(C)COC1)OCCc1c2sc(Cl)c1CO] | 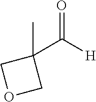 | 712 | LCMS m/z 372.34 [M + H]⁺ |
| 747 | 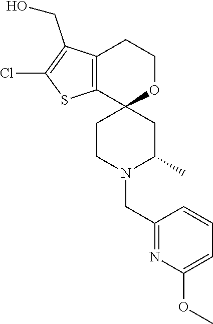 [SMILES: COc1cccc(CN2CC[C@]3(C[C@@H]2C)OCCc2c3sc(Cl)c2CO)n1] | 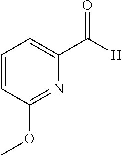 | 712 | LCMS m/z 409.32 [M + H]⁺ |
| 748 | 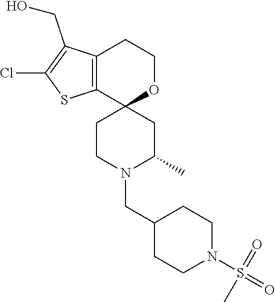 [SMILES: C[C@H]1C[C@@]2(CCN1CC1CCN(S(C)(=O)=O)CC1)OCCc1c2sc(Cl)c1CO] | 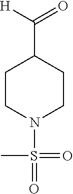 | 712 | LCMS m/z 463.37 [M + H]⁺ |
| 749 | 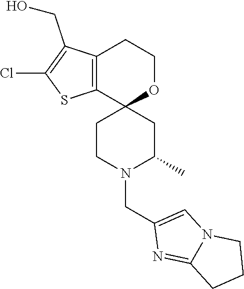 [SMILES: C[C@H]1C[C@@]2(CCN1Cc1cn3c(n1)CCC3)OCCc1c2sc(Cl)c1CO] | 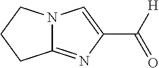 | 712 | LCMS m/z 408.37 [M + H]⁺ |
| 750 | 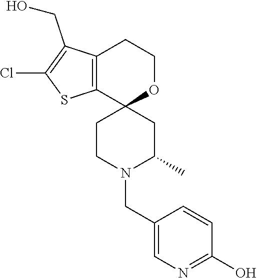 [SMILES: C[C@H]1C[C@@]2(CCN1Cc1ccc(O)nc1)OCCc1c2sc(Cl)c1CO] | 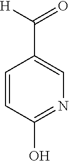 | 712 | LCMS m/z 395.32 [M + H]⁺ |
| 751 | 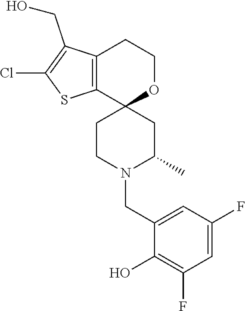 [SMILES: C[C@H]1C[C@@]2(CCN1Cc1cc(F)cc(F)c1O)OCCc1c2sc(Cl)c1CO] | 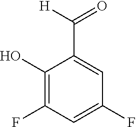 [SMILES: O=Cc1cc(F)cc(F)c1O] | 712 | LCMS m/z 430.32 [M + H]⁺ |
| 752 | 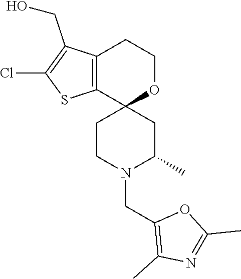 [SMILES: Cc1nc(C)c(CN2CC[C@]3(C[C@@H]2C)OCCc2c3sc(Cl)c2CO)o1] | 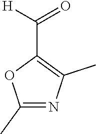 | 712 | LCMS m/z 397.31 [M + H]⁺ |
| 753 | 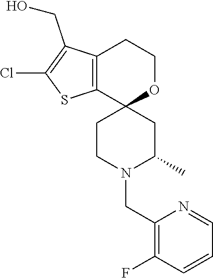 [SMILES: C[C@H]1C[C@@]2(CCN1Cc1ncccc1F)OCCc1c2sc(Cl)c1CO] | 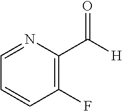 | 712 | ¹H NMR (400 MHZ, DMSO-d₆) δ 9.93 (s, 1H), 8.58 (d, J = 4.7 Hz, 1H), 7.93 (t, J = 9.2 Hz, 1H), 7.64 (dt, J = 8.7, 4.5 Hz, 1H), 4.84 (d, J = 14.8 Hz, 1H), 4.55 (d, J = 15.0 Hz, 1H), 4.33 (s, 2H), 3.98-3.86 (m, 2H), 3.76 (s, 2H), 3.27 (s, 2H), 2.65 (d, J = 5.8 Hz, 2H), 2.26 (d, J = 14.7 Hz, 1H), 2.14 (d, J = 13.6 Hz, 3H), 1.41 (d, J = 6.3 Hz, 3H); LCMS m/z 397.36 [M + H]⁺ |
| 754 | 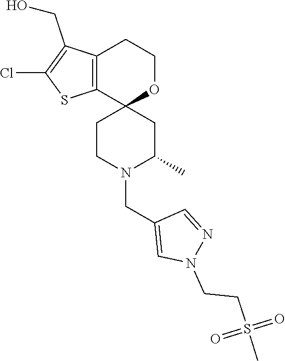 [SMILES: C[C@H]1C[C@@]2(CCN1Cc1cnn(CCS(C)(=O)=O)c1)OCCc1c2sc(Cl)c1CO] | 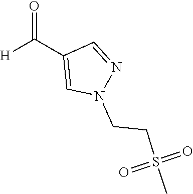 | 7121 | ¹H NMR (400 MHZ, Chloroform-d) δ 7.60- 7.48 (m, 1H), 7.46 (s, 1H), 4.59 (t, J = 6.0 Hz, 2H), 4.54 (s, 2H), 3.96-3.70 (m, 3H), 3.70-3.50 (m, 3H), 2.65 (ddt, J = 9.1, 5.6, 3.3 Hz, 3H), 2.51 (ddd, J = 19.3, 10.7, 4.3 Hz, 2H), 2.46 (s, 3H), 1.98 (tt, J = 14.2, 2.9 Hz, 2H), 1.80 (td, J = 13.3, 4.5 Hz, 1H), 1.60 (dd, J = 13.9, 11.4 Hz, 1H), 1.19 (d, J = 6.2 Hz, 3H); LCMS m/z 474.16 [M + H]⁺ |
| 755 | 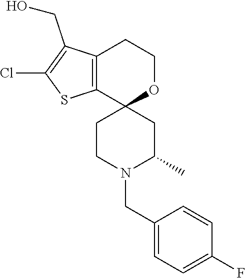 [SMILES: C[C@H]1C[C@@]2(CCN1Cc1ccc(F)cc1)OCCc1c2sc(Cl)c1CO] | 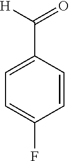 | 713 | ¹H NMR (400 MHZ, DMSO-d₆) δ 9.65 (s, 1H), 7.58 (s, 2H), 7.34 (t, J = 8.6 Hz, 2H), 5.10 (s, 1H), 4.73 (d, J = 12.9 Hz, 1H), 4.32 (s, 2H), 4.16 (s, 1H), 4.01-3.78 (m, 2H), 3.52 (s, 1H), 3.12 (s, 1H), 2.99 (d, J = 7.3 Hz, 1H), 2.68-2.57 (m, 2H), 2.29 (d, J = 15.0 Hz, 1H), 2.15 (d, J = 15.3 Hz, 1H), 1.92 (dd, J = 25.7, 13.1 Hz, 2H), 1.58-1.39 (m, 3H); LCMS m/z 395.9 [M + H]⁺ |
| 756 | 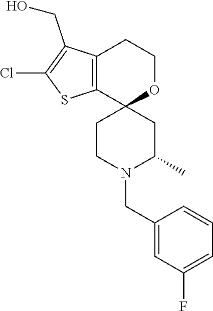 [SMILES: C[C@H]1C[C@@]2(CCN1Cc1cccc(F)c1)OCCc1c2sc(Cl)c1CO] | 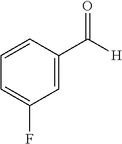 | 713 | ¹H NMR (400 MHZ, DMSO-d₆) δ 9.72 (s, 1H), 7.55 (d, J = 7.5 Hz, 1H), 7.47-7.31 (m, 3H), 5.10 (s, 1H), 4.77 (d, J = 12.7 Hz, 1H), 4.32 (s, 2H), 4.16 (s, 1H), 3.97-3.86 (m, 2H), 3.55 (s, 1H), 3.15 (s, 1H), 3.00 (s, 1H), 2.63 (d, J = 5.7 Hz, 2H), 2.29 (d, J = 15.0 Hz, 1H), 2.15 (d, J = 15.2 Hz, 1H), 1.94 (dd, J = 25.0, 13.4 Hz, 2H), 1.48 (s, 3H); LCMS m/z 395.87 [M + H]⁺ |
| 757 | 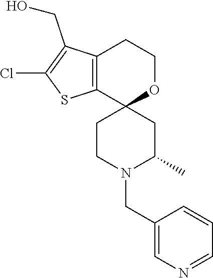 [SMILES: C[C@H]1C[C@@]2(CCN1Cc1cccnc1)OCCc1c2sc(Cl)c1CO] | 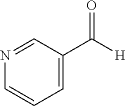 | 713 | ¹H NMR (400 MHZ, DMSO-d₆) δ 9.93 (s, 1H), 8.73 (d, J = 2.3 Hz, 1H), 8.68 (dd, J = 4.9, 1.6 Hz, 1H), 7.99 (d, J = 7.9 Hz, 1H), 7.54 (dd, J = 7.9, 4.9 Hz, 1H), 4.80 (d, J = 13.1 Hz, 1H), 4.32 (s, 2H), 4.22 (d, J = 13.1 Hz, 1H), 3.97-3.86 (m, 2H), 3.21-3.10 (m, 2H), 3.00 (t, J = 6.0 Hz, 1H), 2.64 (t, J = 5.6 Hz, 2H), 2.30 (d, J = 15.0 Hz, 1H), 2.15 (d, J = 14.9 Hz, 1H), 1.92 (dt, J = 22.9, 13.5 Hz, 2H), 1.50 (d, J = 6.4 Hz, 3H); LCMS m/z 378.9 [M + H]⁺ |
| 758 | 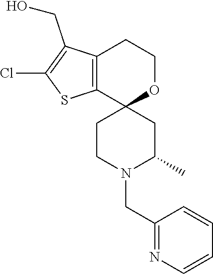 [SMILES: C[C@H]1C[C@@]2(CCN1Cc1ccccn1)OCCc1c2sc(Cl)c1CO] | 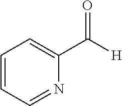 | 713 | ¹H NMR (400 MHZ, DMSO-d₆) δ 10.14- 9.65 (m, 1H), 8.72 (d, J = 4.8 Hz, 1H), 7.95 (td, J = 7.7, 1.8 Hz, 1H), 7.58 (d, J = 7.7 Hz, 1H), 7.50 (dd, J = 7.6, 4.9 Hz, 1H), 5.10 (s, 1H), 4.79 (d, J = 14.2 Hz, 1H), 4.42 (d, J = 14.2 Hz, 1H), 4.33 (s, 2H), 3.91 (dtd, J = 17.4, 11.5, 5.3 Hz, 2H), 3.64 (s, 1H), 3.12 (d, J = 12.4 Hz, 1H), 2.63 (d, J = 5.5 Hz, 2H), 2.23 (d, J = 14.7 Hz, 1H), 2.06 (dt, J = 27.5, 1H NMR; LCMS m/z 12.6 Hz, 3H), 1.40 (d, J = 6.3 Hz, 3H); LCMS m/z 378.9 [M + H]⁺ |

Footnotes:
¹NaBH₃CN was used instead of polymer supported cyanoborohydride. The reaction was heated at 95° C. in DCM. Purification was completed using silica gel chromatography
(Gradient: 0 to 20% MeOH in DCM).

### Compounds 759

### [(2′S, 7R)-2-chloro-1′-[(I-isobutylpyrazol-4-yl)methyl]-2′-methyl-spiro[4,5-dihydrothieno[2,3-c]pyran-7,4′-piperidine]-3-yl]methanol (759)

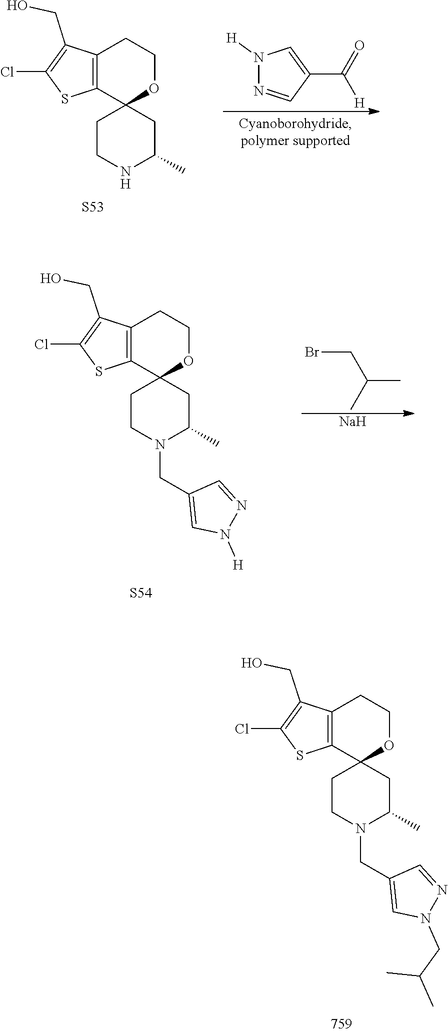

### Step 1. Synthesis of [(2′S,7R)-2-chloro-2′-methyl-1′-(1H-pyrazol-4-ylmethyl)spiro[4,5-dihydrothieno[2,3-c]pyran-7,4′-piperidine]-3-yl]methanol (S54)

[(2′S,7R)-2-chloro-2′-methyl-1′-(1H-pyrazol-4-ylmethyl)spiro[4,5-dihydrothieno[2,3-c]pyran-7,4′-piperidine]-3-yl]methanol S54 was prepared according to standard method A, with the modifications as following: cyanoborohydride, polymer supported (2.0 eq) was used and DCM was used as the solvent. Purification was performed by silica gel chromatography (Gradient: 0-20% MeOH in DCM) to yield the title compound. ¹H NMR (400 MHz, DMSO-d₆) δ 12.68 (s, 1H), 7.49 (d, J=73.3 Hz, 2H), 5.02 (t, J=5.4 Hz, 1H), 4.31 (t, J=5.7 Hz, 2H), 3.81 (q, J=5.6 Hz, 2H), 3.72 (s, 1H), 3.50 (s, 1H), 3.17 (d, J=5.2 Hz, 1H), 2.56 (t, J=5.5 Hz, 3H), 2.38 (s, 1H), 1.92 (d, J=6.4 Hz, 2H), 1.63 (s, 1H), 1.42 (s, 1H), 1.12 (s, 3H). LCMS m/z 368.05 [M+H]⁺.

### Step 2. Synthesis of [(2′S, 7R)-2-chloro-1′-[(1-isobutylpyrazol-4-yl)methyl]-2′-methyl-spiro[4,5-dihydrothieno[2,3-c]pyran-7,4′-piperidine]-3-yl]methanol (759) —Standard Method #C: Pyrazole N-Alkylation

To a mixture of [(2′S,7R)-2-chloro-2′-methyl-1′-(1H-pyrazol-4-ylmethyl)spiro[4,5-dihydrothieno[2,3-c]pyran-7,4′-piperidine]-3-yl]methanol S54 (20 mg, 0.05320 mmol) in DMF (1000 μL) was added NaH (3.5 mg of 60% dispersion in mineral oil, 0.08751 mmol), followed by 1-bromo-2-methyl-propane (20 μL, 0.1839 mmol). After stirring overnight, the mixture was purified with a C18 column (Gradient: 10-100% MeCN in water, 0.1% TFA modifier). The product-containing fractions were pooled and concentrated, rediluted in sat. aq. sodium bicarbonate/DCM to remove TFA. The organic layer was passed over a phase separator and concentrated to yield [(2′S,7R)-2-chloro-1′-[(1-isobutylpyrazol-4-yl)methyl]-2′-methyl-spiro[4,5-dihydrothieno[2,3-c]pyran-7,4′-piperidine]-3-yl]methanol (11 mg, 48%). ¹H NMR (400 MHz, Chloroform-d) δ 7.39 (d, J=0.8 Hz, 1H), 7.31-7.26 (m, 1H), 4.52 (s, 2H), 3.86 (d, J=7.3 Hz, 5H), 3.53 (d, J=14.2 Hz, 1H), 2.66 (dtd, J=10.4, 5.3, 3.3 Hz, 3H), 2.54 (ddt, J=11.2, 6.1, 2.9 Hz, 1H), 2.47 (td, J=12.0, 11.5, 2.5 Hz, 1H), 2.18 (hept, J=6.8 Hz, 1H), 1.95 (d, J=13.7 Hz, 3H), 1.78 (td, J=13.4, 4.4 Hz, 1H), 1.60 (dd, J=13.8, 11.4 Hz, 1H), 1.18 (d, J=6.2 Hz, 3H), 0.89 (dd, J=6.7, 0.9 Hz, 6H). LCMS m/z 424.23 [M+H]⁺.

### Compounds 760-761

Compounds 760-761 (see Table 32) were prepared in a single step from intermediate S54 using method as in the preparation of compound 759. Alkyl halides were obtained from commercial sources. Any modifications to methods are noted in Table 32 and accompanying footnotes.

TABLE 32
Structure and physicochemical data for compounds 760-761

| | | Alkyl Halide | | |
|---|---|---|---|---|
| Cmpd | Structure | Reagent | Method | ¹H NMR; LCMS m/z [M + H]⁺ |
| 760 | 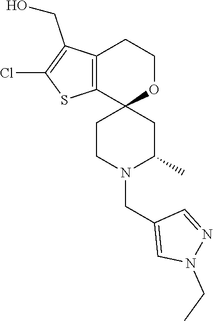 [SMILES: CCn1cc(CN2CC[C@]3(C[C@@H]2C)OCCc2c3sc(Cl)c2CO)cn1] | 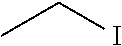 | 759 | ¹H NMR (400 MHz, DMSO-d₆) δ 10.97 (s, 1H), 7.97 (s, 1H), 7.63 (s, 1H), 4.32 (s, 2H), 4.16 (q, J = 6.9 Hz, 3H), 3.86 (q, J = 6.6, 6.2 Hz, 2H), 3.27-3.04 (m, 3H), 2.98 (d, J = 13.7 Hz, 1H), 2.61 (t, J = 5.4 Hz, 2H), 2.27- 2.05 (m, 4H), 1.46 (d, J = 6.3 Hz, 3H), 1.37 (t, J = 7.2 Hz, 4H); LCMS m/z 396.36 [M + H]⁺. |
| 761 | 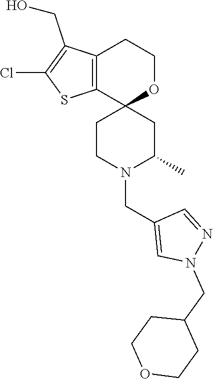 [SMILES: C[C@H]1C[C@@]2(CCN1Cc1cnn(CC3CCOCC3)c1)OCCc1c2sc(Cl)c1CO] | 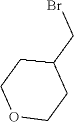 | 759 | ¹H NMR (400 MHz, DMSO-d₆) δ 11.00 (s, 1H), 7.96 (s, 1H), 7.65 (s, 1H), 4.32 (s, 2H), 4.19 (dd, J = 14.1, 5.2 Hz, 1H), 4.04 (d, J = 7.1 Hz, 2H), 3.91-3.73 (m, 4H), 3.29-3.11 (m, 5H), 2.61 (d, J = 5.3 Hz, 2H), 2.28-1.94 (m, 5H), 1.45 (d, J = 6.3 Hz, 3H), 1.36 (d, J = 12.9 Hz, 2H), 1.27-1.15 (m, 3H); LCMS m/z 466.39 [M + H]⁺. |

### Compound 762

### (2′S, 7R)-2-chloro-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](762)

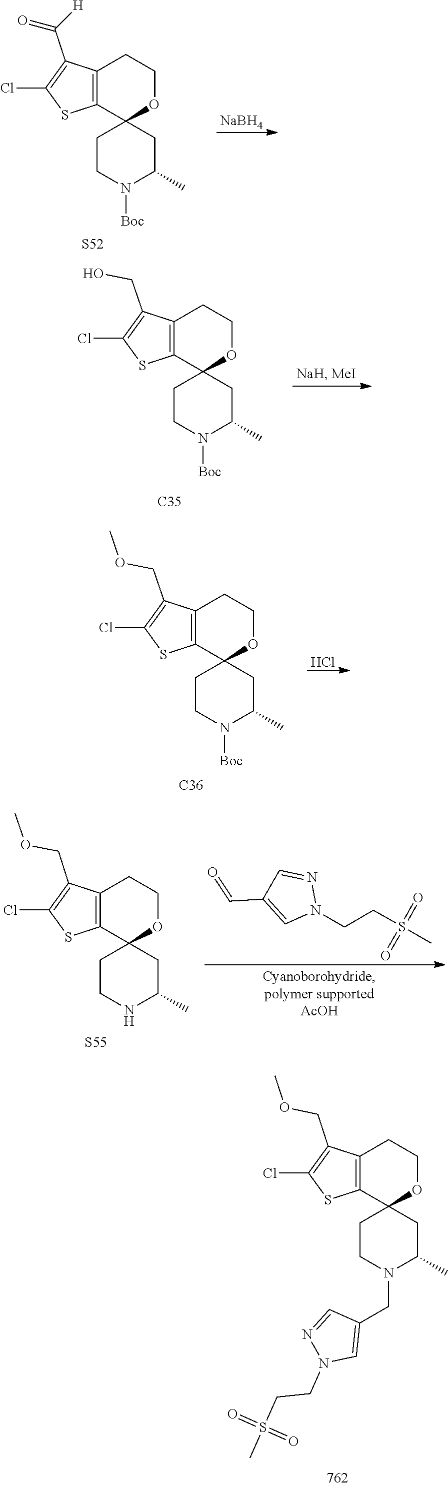

### Step 1. Synthesis of tert-butyl (2′S, 7R)-2-chloro-3-(hydroxymethyl)-2′-methyl-spiro[4,5-dihydrothieno[2,3-c]pyran-7,4′-piperidine]-1′-carboxylate (C35)

To a mixture of tert-butyl (2′S,7R)-2-chloro-3-formyl-2′-methyl-spiro[4,5-dihydrothieno[2,3-c]pyran-7,4′-piperidine]-1′-carboxylate S52 (400 mg, 1.023 mmol) in tetrahydrofuran (8 mL) was added NaBH₄ (13 μL, 0.3247 mmol) followed by MeOH (160 μL) and the reaction was heated to 50° C. After 10 min, the mixture was quenched with sat. aq. sodium bicarbonate and EtOAc. The organic layer was separated, rinsed with water and brine. The organic layer was dried with sodium sulfate, filtered and concentrated to provide tert-butyl (2′S,7R)-2-chloro-3-(hydroxymethyl)-2′-methyl-spiro[4,5-dihydrothieno[2,3-c]pyran-7,4′-piperidine]-1′-carboxylate. LCMS m/z 387.9 [M+H]⁺. The material was used in the next step without further purification.

### Step 2. Synthesis of tert-butyl (2′S, 7R)-2-chloro-3-(methoxymethyl)-2′-methyl-spiro[4,5-dihydrothieno[2,3-c]pyran-7,4′-piperidine]-1′-carboxylate (C36)

Tert-butyl (2′S,7R)-2-chloro-3-(hydroxymethyl)-2′-methyl-spiro[4,5-dihydrothieno[2,3-c]pyran-7,4′-piperidine]-1′-carboxylate C35 (400 mg, 1.031 mmol) was dissolved in THF (11 mL) and MeI (192 μL, 3.084 mmol) and NaH (74 mg, 3.084 mmol) were added. The mixture was stirred at room temperature for 48. Then saturated NH₄Cl aqueous solution was added to quench the reaction and the solution was extracted with DCM, dried with sodium sulfate and filtered to give crude product. Purification by silica gel chromatography (Gradient: 0-20% EtOAc in heptane) yielded tert-butyl (2′S,7R)-2-chloro-3-(methoxymethyl)-2′-methyl-spiro[4,5-dihydrothieno[2,3-c]pyran-7,4′-piperidine]-1′-carboxylate (340 mg, 82%). LCMS m/z 402.04 [M+H]⁺.

### Step 3. Synthesis of (2′S, 7R)-2-chloro-3-(methoxymethyl)-2′-methyl-spiro[4,5-dihydrothieno[2,3-c]pyran-7,4′-piperidine](S55)

Tert-butyl (2′S,7R)-2-chloro-3-(methoxymethyl)-2′-methyl-spiro[4,5-dihydrothieno[2,3-c]pyran-7,4′-piperidine]-1′-carboxylate C36 (113 mg, 0.2811 mmol) was dissolved in HCl (703 μL of 4 M, 2.812 mmol). After 20 min, the solvent was removed to provide (2′S,7R)-2-chloro-3-(methoxymethyl)-2′-methyl-spiro[4,5-dihydrothieno[2,3-c]pyran-7,4′-piperidine]. LCMS m/z 302.22 [M+H]⁺. The material was used in the next step without further purification.

### Step 4. Synthesis of (2′S, 7R)-2-chloro-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](762)

(2′S,7R)-2-chloro-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine]S55 (70 mg, 0.17 mmol) and 1-(2-methylsulfonylethyl)pyrazole-4-carbaldehyde (57 mg, 0.2819 mmol) were dissolved in DCM (3 mL). Acetic acid (50.7 mg, 0.8443 mmol) was added to the solution. The solution was transferred to a microwave tube and cyanoborohydride, polymer supported (253 mg of 2 mmol/g, 0.5060 mmol) was added. The tube was capped and heated to 110° C. in a microwave reactor for 45 min. The borohydride resin was filtered off and the solvent was removed in vacuo. Purification by reversed-phase HPLC. Method: C18 Waters Sunfire column (30×150 mm, 5 micron). Gradient: MeCN in H₂O with 0.1% trifluoroacetic acid afforded (2′S,7R)-2-chloro-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](Trifluoroacetate salt) (38.2 mg, 37%). ¹H NMR (300 MHz, Chloroform-d) δ 7.84 (s, 1H), 7.64 (s, 1H), 4.81-4.51 (m, 3H), 4.34 (s, 2H), 4.10-3.60 (m, 5H), 3.35 (s, 3H), 2.99 (s, 4H), 2.86-2.61 (m, 5H), 2.45-2.05 (m, 4H), 1.58 (d, J=6.5 Hz, 3H). LCMS m/z 488.21 [M+H]⁺.

### Compound 763

### (2′S, 7R)-2-(difluoromethyl)-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](763)

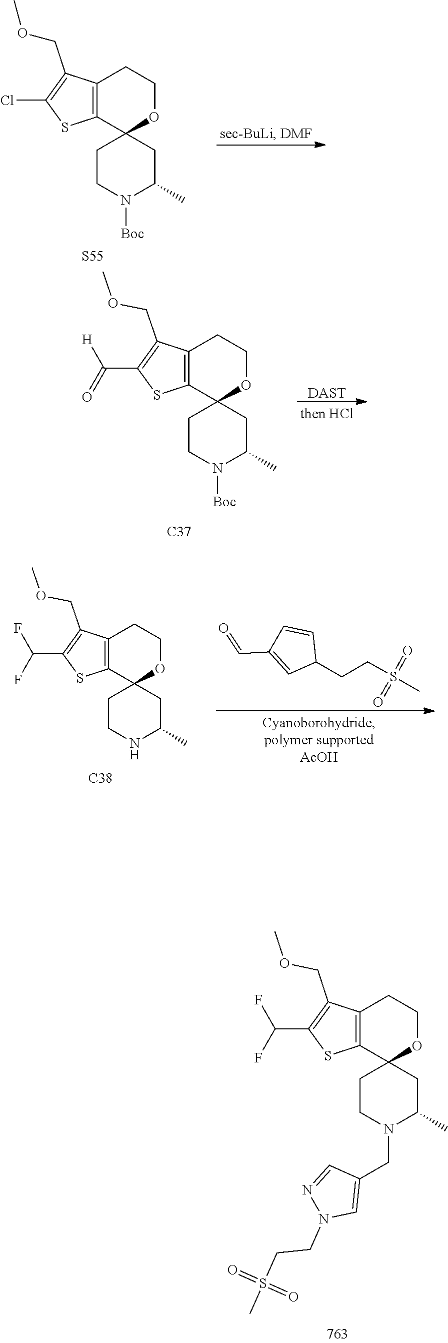

### Step 1. Synthesis of tert-butyl (2′S, 7R)-2-formyl-3-(methoxymethyl)-2′-methyl-spiro[4,5-dihydrothieno[2,3-c]pyran-7,4′-piperidine]-1′-carboxylate (C37)

A mixture of tert-butyl (2′S,7R)-2-chloro-3-(methoxymethyl)-2′-methyl-spiro[4,5-dihydrothieno[2,3-c]pyran-7,4′-piperidine]-1′-carboxylate S55 (226 mg, 0.5615 mmol) in THF (5 mL) in an oven dried vial was cooled to −70° C. At this time, sec-butyllithium (570 μL of 1.25 M, 0.7125 mmol) in cyclohexane was added dropwise, and stirred at this temperature. After 20 min, N,N-dimethylformamide (222 μL, 2.867 mmol) was added. The mixture was allowed to warm to room temperature. The reaction mixture was stirred at this temperature overnight. To this mixture was added sat. aq. ammonium chloride (6 mL), and the mixture was warmed to room temperature. At this time, the mixture was diluted with Et₂O, followed by water. The organic layer was washed with brine. The organic layer was dried with MgSO₄, filtered, and concentrated in vacuo. The mixture was purified by silica gel chromatography (Gradient: 0-25% EtOAc in heptane) to give tert-butyl (2′S,7R)-2-formyl-3-(methoxymethyl)-2′-methyl-spiro[4,5-dihydrothieno[2,3-c]pyran-7,4′-piperidine]-1′-carboxylate (75 mg, 34%). ¹H NMR (300 MHz, Methanol-d₄) δ 10.13 (s, 1H), 4.68 (s, 2H), 4.11-3.73 (m, 4H), 3.41 (s, 3H), 2.65 (t, J=5.6 Hz, 2H), 2.43-2.01 (m, 2H), 1.99-1.60 (m, 2H), 1.48 (s, 10H), 1.22 (d, J=6.5 Hz, 3H). LCMS m/z 396.05 [M+H]⁺.

### Step 2. Synthesis of (2′S,7R)-2-(difluoromethyl)-3-(methoxymethyl)-2′-methyl-spiro[4,5-dihydrothieno[2,3-c]pyran-7,4′-piperidine](C38)

To a solution of tert-butyl (2′S,7R)-2-formyl-3-(methoxymethyl)-2′-methyl-spiro[4,5-dihydrothieno[2,3-c]pyran-7,4′-piperidine]-1′-carboxylate C37 (70 mg, 0.1763 mmol) in DCM (700 μL) was added DAST (116 μL, 0.8780 mmol) and the reaction was heated to 40° C. After 4 h, additional DAST (116 μL, 0.8780 mmol) was added. After stirring overnight, the solution was diluted with DCM and washed with saturated NaHCO₃ solution, extracted with DCM (2×) and washed with brine. The solvent was removed to give the crude product. The crude material was redissolved in dioxane (2 mL) and HCl (1.3 mL of 4 M, 5.200 mmol) was added. After 30 min, the solvent was removed and purified by reversed-phase HPLC. Method: C18 Waters Sunfire column (30×150 mm, 5 micron). Gradient: MeCN in H₂O with 0.1% trifluoroacetic acid. Product was isolated as (2′S,7R)-2-(difluoromethyl)-3-(methoxymethyl)-2′-methyl-spiro[4,5-dihydrothieno[2,3-c]pyran-7,4′-piperidine](Trifluoroacetate salt) (43 mg, 55%). ¹H NMR (300 MHz, Chloroform-d) δ 9.48 (s, 1H), 8.80 (s, 1H), 7.04 (t, J=55.5 Hz, 1H), 4.43 (d, J=1.3 Hz, 2H), 4.34-3.88 (m, 4H), 3.65 (s, 1H), 3.41 (s, 5H), 2.67 (t, J=5.4 Hz, 2H), 2.28-1.93 (m, 4H), 1.38 (d, J=6.5 Hz, 3H). LCMS m/z 318.25 [M+H]⁺.

### Step 3. Synthesis of (2′S, 7R)-2-(difluoromethyl)-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](763)

(2′S,7R)-2-(difluoromethyl)-3-(methoxymethyl)-2′-methyl-spiro[4,5-dihydrothieno[2,3-c]pyran-7,4′-piperidine](Trifluoroacetate salt) C38 (11.1 mg, 0.02496 mmol) and 1-(2-methylsulfonylethyl)pyrazole-4-carbaldehyde (6.5 mg, 0.03214 mmol) were dissolved in DCM (2 mL). Acetic acid (4.5 mg, 0.07494 mmol) was added to the solution. The solution was transferred to a microwave tube (5 mL) and cyanoborohydride, polymer supported (62 mg of 2 mmol/g, 0.1240 mmol) was added. The solution was capped and heated to 110° C. in a microwave reactor for 45 min. The solution was filtered, the solvent was removed. Purification by reversed-phase HPLC. Method: C18 Waters Sunfire column (30×150 mm, 5 micron). Gradient: MeCN in H₂O with 0.1% trifluoroacetic acid afforded (2′S,7R)-2-(difluoromethyl)-3-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](Trifluoroacetate salt) (5.4 mg, 26%). LCMS m/z 504.09 [M+H]⁺.

### Compound 764

### [(2′S, 7R)-2-(difluoromethyl)-2′-methyl-1′-[[1-(2-methylsulfonylethyl)pyrazol-4-yl]methyl]spiro[4,5-dihydrothieno[2,3-c]pyran-7,4′-piperidine]-3-yl]methanol (764)

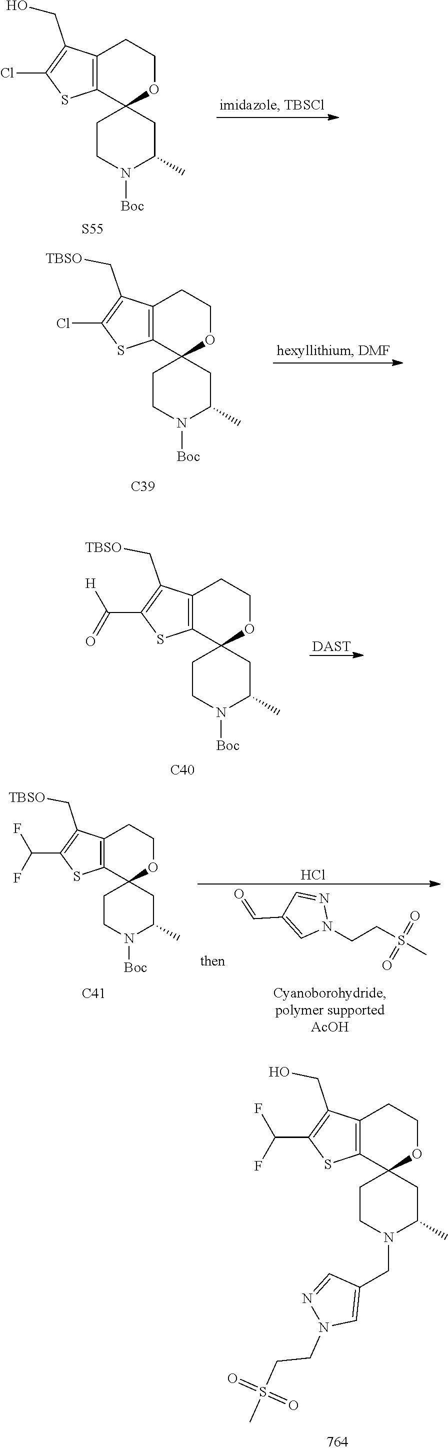

### Step 1. Synthesis of tert-butyl (2′S,7R)-3-[[tert-butyl(dimethyl)silyl]oxymethyl]-2-chloro-2′-methyl-spiro[4,5-dihydrothieno[2,3-c]pyran-7,4′-piperidine]-1′-carboxylate (C39)

To a solution of tert-butyl (2′S,7R)-2-chloro-3-(hydroxymethyl)-2′-methyl-spiro[4,5-dihydrothieno[2,3-c]pyran-7,4′-piperidine]-1′-carboxylate S55 (396 mg, 1.021 mmol) in DCM (8 mL), was added imidazole (400 mg, 5.876 mmol), and TBSCl (649 mg, 4.306 mmol). The mixture was stirred for 40 min and then it was quenched with 1 N HCl, diluted with DCM and separated by a phase separator. The mixture was purified by silica gel chromatography (Gradient: 0-10% EtOAc in heptane) to provide tert-butyl (2′S,7R)-3-[[tert-butyl(dimethyl)silyl]oxymethyl]-2-chloro-2′-methyl-spiro[4,5-dihydrothieno[2,3-c]pyran-7,4′-piperidine]-1′-carboxylate (385 mg, 65%). LCMS m/z 502.31 [M+H]⁺.

### Step 2. Synthesis of tert-butyl (2′S,7R)-3-[[tert-butyl(dimethyl)silyl]oxymethyl]-2-formyl-2′-methyl-spiro[4,5-dihydrothieno[2,3-c]pyran-7,4′-piperidine]-1′-carboxylate (C40)

A mixture of tert-butyl (2′S,7R)-3-[[tert-butyl(dimethyl)silyl]oxymethyl]-2-chloro-2′-methyl-spiro[4,5-dihydrothieno[2,3-c]pyran-7,4′-piperidine]-1′-carboxylate C39 (1070 mg, 2.070 mmol) in THF (25 mL) in a flame dried flask was cooled to −78° C. At this time, hexyllithium (1.8 mL of 2.3 M, 4.140 mmol) in hexane was added, dropwise, and stirred at this temperature for 10 min. At this time, N,N-dimethylformamide (500 μL, 6.457 mmol) was added, and the mixture was stirred for another 10 min. The mixture was quenched with ˜7 M aq. ammonium chloride (3 mL). The mixture was allowed to warm to room temperature. The reaction was diluted with Et₂O (100 mL) and water (100 mL). The organic layer was separated and washed with water (2×100 mL) and brine (100 mL), dried with sodium sulfate, and concentrated. The mixture was purified by silica gel chromatography (Gradient: 0-30% EtOAc in heptane) to provide tert-butyl (2′S,7R)-3-[[tert-butyl(dimethyl)silyl]oxymethyl]-2-formyl-2′-methyl-spiro[4,5-dihydrothieno[2,3-c]pyran-7,4′-piperidine]-1′-carboxylate (771 mg, 75%). ¹H NMR (400 MHz, Chloroform-d) δ 10.22 (s, 1H), 4.93-4.81 (m, 2H), 4.01 (tt, J=11.7, 6.4 Hz, 1H), 3.96-3.85 (m, 2H), 3.76 (ddd, J=13.9, 6.1, 4.6 Hz, 1H), 3.34 (ddd, J=14.1, 9.0, 5.3 Hz, 1H), 2.69-2.55 (m, 2H), 2.26-2.16 (m, 1H), 2.11 (ddd, J=14.2, 5.2, 1.9 Hz, 1H), 1.83 (ddd, J=14.5, 5.3, 4.6 Hz, 1H), 1.75 (dd, J=14.3, 11.0 Hz, 1H), 1.48 (s, 9H), 1.26 (d, J=6.6 Hz, 3H), 0.90 (s, 9H), 0.10 (d, J=0.8 Hz, 6H). LCMS m/z 496.21 [M+H]⁺.

### Step 3. Synthesis of tert-butyl (2′S,7R)-3-[[tert-butyl(dimethyl)silyl]oxymethyl]-2-(difluoromethyl)-2′-methyl-spiro[4,5-dihydrothieno[2,3-c]pyran-7,4′-piperidine]-1′-carboxylate (C41)

To a mixture of tert-butyl (2′S,7R)-3-[[tert-butyl(dimethyl)silyl]oxymethyl]-2-formyl-2′-methyl-spiro[4,5-dihydrothieno[2,3-c]pyran-7,4′-piperidine]-1′-carboxylate C40 (100 mg, 0.1990 mmol) in DCM (500 μL) was added DAST (75 μL, 0.5677 mmol) and the mixture was heated to 40° C. After 1 h, additional DAST (75 μL, 0.5677 mmol) was added and the reaction was stirred overnight. The mixture was slowly added to a stirring saturated solution of aqueous sodium bicarbonate at 0° C. Upon quenching, additional DCM (20 mL) was added and the organic layer was separated and concentrated. The crude material was purified by silica gel chromatography (Gradient: 0-20% EtOAc in heptane) to provide tert-butyl (2′S,7R)-3-[[tert-butyl(dimethyl)silyl]oxymethyl]-2-(difluoromethyl)-2′-methyl-spiro[4,5-dihydrothieno[2,3-c]pyran-7,4′-piperidine]-1′-carboxylate. The material was used in the next step without further purification.

### Step 4. Synthesis of [(2′S, 7R)-2-(difluoromethyl)-2′-methyl-1′-[[1-(2-methylsulfonylethyl)pyrazol-4-yl]methyl]spiro[4,5-dihydrothieno[2,3-c]pyran-7,4′-piperidine]-3-yl]methanol (764)

To the product from the previous step (C41) was added HCl (550 μL of 4 M in dioxane, 2.200 mmol). After 25 min, the mixture was diluted with Et₂O and then concentrated. DCM was added, it was concentrated once again, and then diluted with DCM (1 mL). Methanol (0.1 mL) was added, followed by 1-(2-methylsulfonylethyl)pyrazole-4-carbaldehyde (75 mg, 0.3193 mmol) and cyanoborohydride, polymer supported (150 mg of 2 mmol/g, 0.3000 mmol). The mixture was heated to 80° C. After 4 h and 35 min, the mixture was cooled to room temperature, filtered and the polymer was rinsed with additional methanol. The mixture was concentrated, purified by silica gel chromatography (Gradient: 0-10% MeOH in DCM) to yield [(2′S,7R)-2-(difluoromethyl)-2′-methyl-1′-[[1-(2-methylsulfonylethyl)pyrazol-4-yl]methyl]spiro[4,5-dihydrothieno[2,3-c]pyran-7,4′-piperidine]-3-yl]methanol (17.4 mg, 18%). ¹H NMR (400 MHz, Chloroform-d) δ 7.43 (d, J=0.7 Hz, 1H), 7.39 (s, 1H), 6.99 (t, J=55.8 Hz, 1H), 4.57 (t, J=1.3 Hz, 2H), 4.52 (dd, J=6.6, 5.5 Hz, 2H), 3.87-3.71 (m, 3H), 3.60-3.50 (m, 3H), 2.63-2.53 (m, 3H), 2.53-2.40 (m, 2H), 2.38 (d, J=1.6 Hz, 3H), 1.98-1.88 (m, 2H), 1.85-1.77 (m, 2H), 1.61 (dd, J=13.9, 11.4 Hz, 1H), 1.12 (d, J=6.2 Hz, 3H). LCMS m/z 490.09 [M+H]⁺.

### Compound 765

### [(2′S, 7R)-2,2′-dimethyl-1′-[[1-(2-methylsulfonylethyl)pyrazol-4-yl]methyl]spiro[4,5-dihydrothieno[2,3-c]pyran-7,4′-piperidine]-3-yl]methanol (765)

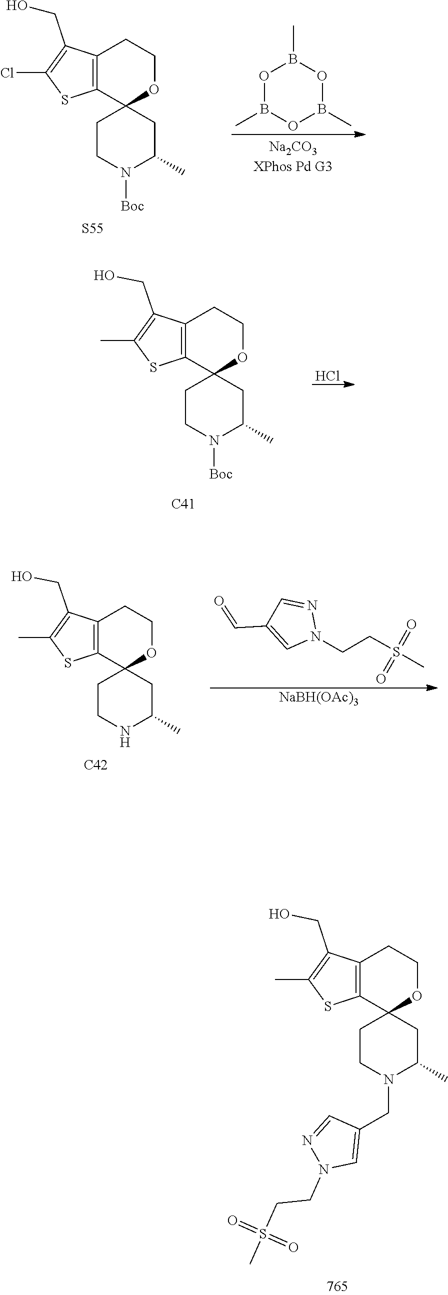

### Step 1. Synthesis of tert-butyl (2′S, 7R)-3-(hydroxymethyl)-2,2′-dimethyl-spiro[4,5-dihydrothieno[2,3-c]pyran-7,4′-piperidine]-1′-carboxylate (C41)

To a 1-dram vial was added tert-butyl (2′S,7R)-2-chloro-3-(hydroxymethyl)-2′-methyl-spiro[4,5-dihydrothieno[2,3-c]pyran-7,4′-piperidine]-1′-carboxylate S55 (97 mg, 0.2501 mmol), sodium carbonate (138 mg, 1.302 mmol), XPhos Pd G3 (21 mg, 0.02481 mmol) and the vial was purged with N₂ for 15 min. Then dioxane (700 μL), water (700 μL, degassed) and 2,4,6-trimethyl-1,3,5,2,4,6-trioxatriborinane (110 μL, 0.7869 mmol) were added. The reaction mixture was stirred at 80° C. After 3 h, the reaction was quenched with saturated NaHCO₃ solution and extracted with EtOAc (×4). The combined organic layer was dried over Na₂SO₄, filtered, and concentrated. The crude material was purified by silica gel chromatography (Gradient: 0 to 60% EtOAc in heptane) to yield tert-butyl (2′S,7R)-3-(hydroxymethyl)-2,2′-dimethyl-spiro[4,5-dihydrothieno[2,3-c]pyran-7,4′-piperidine]-1′-carboxylate (74.7 mg, 81%).

### Step 2. Synthesis of [(2′S, 7R)-2,2′-dimethylspiro[4,5-dihydrothieno[2,3-c]pyran-7,4′-piperidine]-3-yl]methanol (C42)

To tert-butyl (2′S,7R)-3-(hydroxymethyl)-2,2′-dimethyl-spiro[4,5-dihydrothieno[2,3-c]pyran-7,4′-piperidine]-1′-carboxylate C41 (74.7 mg, 0.203 mmol) in DCM (1 mL) was added HCl (650 μL of 4 M, 2.600 mmol), and the reaction was left at room temperature. After 3 h, the reaction was quenched with saturated NaHCO₃ solution and extracted with DCM (×6). The combined organic layer was dried over Na₂SO₄, filtered, and concentrated to provide crude material [(2′S,7R)-2,2′-dimethylspiro[4,5-dihydrothieno[2,3-c]pyran-7,4′-piperidine]-3-yl]methanol (43.4 mg, 65%).

### Step 3. Synthesis of [(2′S, 7R)-2,2′-dimethyl-1′-[[1-(2-methylsulfonylethyl)pyrazol-4-yl]methyl]spiro[4,5-dihydrothieno[2,3-c]pyran-7,4′-piperidine]-3-yl]methanol (765)

To the crude material from step 2 (C42) and 1-(2-methylsulfonylethyl)pyrazole-4-carbaldehyde (76 mg, 0.3758 mmol) in DCM (970 μL), triacetoxy(sodio)boron (126 mg, 0.5973 mmol) was added. After 16 h, the reaction was quenched with saturated NaHCO₃ solution and extracted with EtOAc (×4). The combined organic layer was dried over Na₂SO₄, filtered, and concentrated. The crude material was purified by silica gel chromatography (Gradient: 0 to 20% MeOH in DCM) to yield [(2′S,7R)-2,2′-dimethyl-1′-[[1-(2-methylsulfonylethyl)pyrazol-4-yl]methyl]spiro[4,5-dihydrothieno[2,3-c]pyran-7,4′-piperidine]-3-yl]methanol (30.8 mg, 25% over 3 steps). ¹H NMR (300 MHz, Chloroform-d) δ 7.50 (s, 1H), 7.45 (s, 1H), 4.66-4.53 (m, 2H), 4.49 (s, 2H), 3.83 (dd, J=13.5, 8.5 Hz, 3H), 3.73-3.56 (m, 3H), 2.71-2.47 (m, 4H), 2.45 (s, 3H), 2.42 (s, 3H), 2.09-1.76 (m, 3H), 1.66 (t, J=12.8 Hz, 3H), 1.19 (d, J=6.2 Hz, 3H). LCMS m/z 454.17 [M+H]⁺.

### Preparation S56

### (2′S,4S,7R)-2-chloro-2′-methyl-spiro[4,5-dihydrothieno[2,3-c]pyran-7,4′-piperidine]-4-ol (S56)

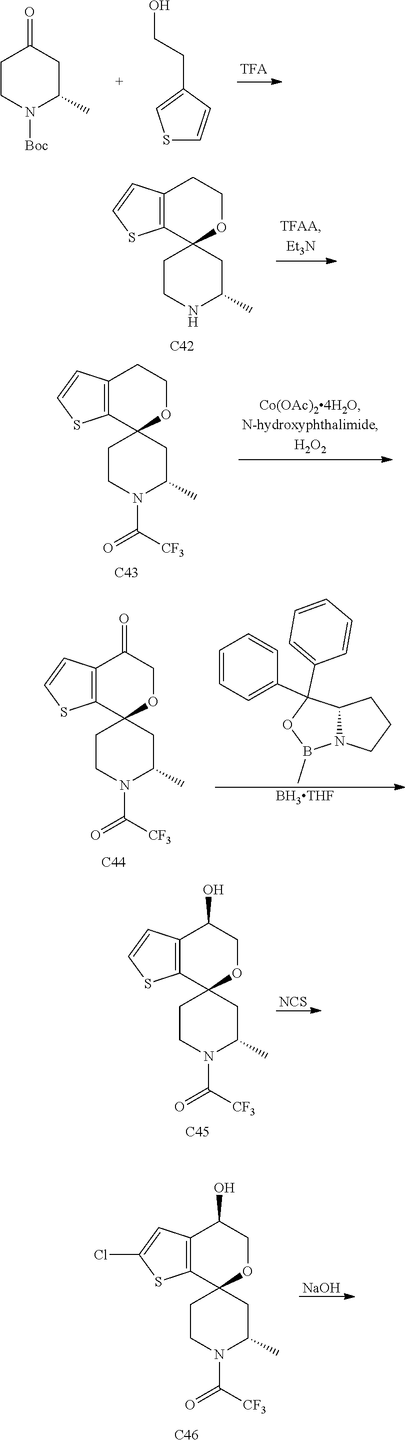

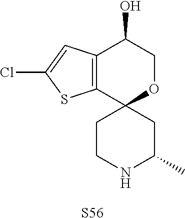 [SMILES: C[C@H]1C[C@@]2(CCN1)OC[C@H](O)c1cc(Cl)sc12 ; "S56"]

### Step 1. Synthesis of (2′S, 7R)-2′-methylspiro[4,5-dihydrothieno[2,3-c]pyran-7,4′-piperidine](C42)

A 3-neck round bottom flask equipped with a nitrogen inlet, addition funnel, mechanical stirrer, and internal temperature probe was charged with a solution of tert-butyl (2S)-2-methyl-4-oxo-piperidine-1-carboxylate (144.1 g, 675.7 mmol) in DCM (1.30 L). The solution was cooled to 5° C. and TFA (1.10 L) was added dropwise at such a rate as to maintain the internal temperature below 10° C. The resulting solution was stirred until complete starting material consumption was observed by TLC and 1H NMR. The vessel was then evacuated with the house vacuum for 5 min and then sparged with a nitrogen (balloon). The mixture was cooled to −62° C. which caused the solution to become a thick slurry. 2-(3-thienyl)ethanol (91.2 g, 711.4 mmol) was added dropwise ensuring the internal temperature did not surpass −55° C. The mixture was allowed to warm to ambient temperature as the cooling bath expired overnight.

After overnight stirring, the mixture was concentrated and taken up in DCM (1 L) and concentrated in vacuo. The residue was taken up in DCM (500 mL) and 2 M aqueous sodium hydroxide was added with stirring until the aqueous layer reached pH 7-8. The layers were separated, and the aqueous phase was extracted with DCM (2×100 mL). The combined organic extracts were washed with brine, dried over sodium sulfate, filtered, and concentrated to provide (2′S,7R)-2′-methylspiro[4,5-dihydrothieno[2,3-c]pyran-7,4′-piperidine](150.9 g, 100%). LCMS m/z 224.04 [M+H]⁺. The material was carried to the next step without further purification.

### Step 2. Synthesis of 2,2,2-trifluoro-1-[(2′S, 7R)-2′-methylspiro[4,5-dihydrothieno[2,3-c]pyran-7,4′-piperidine]-1′-yl]ethanone (C43)

A 3-neck round bottom flask equipped with a nitrogen inlet, addition funnel, mechanical stirrer, and internal temperature probe was charged with (2′S,7R)-2′-methylspiro[4,5-dihydrothieno[2,3-c]pyran-7,4′-piperidine] C42 (150.9 g) in DCM (1.3 L) and cooled to 5° C. Et₃N (0.300 L, 2.152 mol) and TFAA (0.100 L, 719.4 mmol) were sequentially added dropwise to the solution. After 20 min, the cooling bath was removed. Upon complete amine consumption, the reaction was quenched with 10% aqueous citric acid (400 mL) and the layers were separated. The aqueous portion was extracted with DCM (100 mL). The combined organic layers were twice washed with saturated aqueous sodium bicarbonate (300 mL), dried over sodium sulfate, filtered, and concentrated. The crude residue was twice taken up in heptane (1 L) and concentrated to afford a yellow oil. The oil was subsequently taken up in 5% isopropyl acetate in heptane and heated until the solution was homogeneous. Once homogeneity was achieved, the stirring was turned off to allow the insoluble materials to settle, the solution was cannulated into a side-arm Erlenmeyer flask, and allowed to cool to room temperature. At this point, seeds were introduced (from a test crop from the same lot) and the solution was gently stirred overnight under ambient conditions. The solids were collected were purified by silica gel chromatography (Gradient: 0-15% tert-butyl methyl ether in heptane) to afford the title compound as an oil that solidifies to a white solid upon standing. 2,2,2-trifluoro-1-[(2′S,7R)-2′-methylspiro[4,5-dihydrothieno[2,3-c]pyran-7,4′-piperidine]-1′-yl]ethanone (142.3 g, 63%) ¹H NMR (300 MHz, Chloroform-d) δ 7.16 (d, J=5.0 Hz, 1H), 6.75 (d, J=5.0 Hz, 1H), 4.50-4.27 (m, 1H), 3.85 (t, J=5.5 Hz, 3H), 3.44 (s, 1H), 2.66 (t, J=5.5 Hz, 2H), 2.31 (ddd, J=14.3, 6.3, 1.9 Hz, 2H), 1.99 (ddd, J=14.8, 6.0, 2.4 Hz, 1H), 1.83 (dd, J=14.4, 10.8 Hz, 1H), 1.30 (d, J=6.5 Hz, 3H). ¹⁹F NMR (282 MHz, Chloroform-d) δ−69.75. LCMS m/z 320.1 [M+H]⁺.

### Step 3. Synthesis of (2S,4R)-2-methyl-1-(2,2,2-trifluoroacetyl)spiro[piperidine-4,7′-thieno[2,3-c]pyran]-4′-one (C44)

To a mixture under air of 2,2,2-trifluoro-1-[(2′S,7R)-2′-methylspiro[4,5-dihydrothieno[2,3-c]pyran-7,4′-piperidine]-1′-yl]ethanone C43 (5000 mg, 14.87 mmol) in acetonitrile (300 mL) was added N-hydroxyphthalimide (3.3 g, 20.23 mmol) and cobalt(II) diacetate tetrahydrate (200 mg, 0.8029 mmol), and then aqueous hydrogen peroxide (3.2 mL of 30% w/w, 31.33 mmol). The yellow mixture was stirred at 50° C. After 30 h, the mixture was cooled to room temperature and concentrated to about 2 vol. The solid was diluted with MTBE (100 mL), which precipitated a tan solid that was filtered and rinsed with additional MTBE. The green filtrate was washed with sat. aq. sodium bicarbonate (100 mL). The layers were split and the organic layer was washed with 3 additional washes of sodium bicarbonate solution, until the organic layer showed little to no NHPI. The organic layer was washed a final time with brine (100 mL), dried with sodium sulfate, filtered, and concentrated to dryness to yield 4.9 g of crude material. The crude material was purified using silica gel chromatography (Gradient: 0-30% EtOAc in heptane) to yield (2S,4R)-2-methyl-1-(2,2,2-trifluoroacetyl)spiro[piperidine-4,7′-thieno[2,3-c]pyran]-4′-one (2.78 g, 53%). ¹H NMR (400 MHz, DMSO-d₆) δ 7.61 (d, J=5.2 Hz, 1H), 7.31 (d, J=5.2 Hz, 1H), 4.39 (d, J=17.2 Hz, 1H), 4.32-4.08 (m, 2H), 3.74 (s, 2H), 2.56 (d, J=14.7 Hz, 1H), 2.42-2.26 (m, 1H), 2.10 (d, J=15.1 Hz, 2H), 1.20 (s, 3H). LCMS m/z 334.01 [M+H]⁺.

### Step 4. Synthesis of 2,2,2-trifluoro-1-[(2′S,4S,7R)-4-hydroxy-2′-methyl-spiro[4,5-dihydrothieno[2,3-c]pyran-7,4′-piperidine]-1′-yl]ethenone (C45)

To a mixture of a toluene solution of (3aS)-1-methyl-3,3-diphenyl-3a,4,5,6-tetrahydropyrrolo[1,2-c][1,3,2]oxazaborole (1250 μL of 1 M, 1.250 mmol) in MTBE (40 mL) at 0° C. was added borane tetrahydrofuran (12 mL of 1 M, 12.00 mmol). After 10 min, a solution of (2S,4R)-2-methyl-1-(2,2,2-trifluoroacetyl)spiro[piperidine-4,7′-thieno[2,3-c]pyran]-4′-one C44 (2800 mg, 8.182 mmol) in MTBE (20 mL) was added via an addition funnel. After a total 50 min, additional borane was added (3 mL of 1 M, 3.00 mmol). After 10 min, the mixture was quenched with 1N aq. HCl (50 mL). The organic layer was washed with brine, dried with sodium sulfate and filtered. The mixture was concentrated and purified by silica gel chromatography (Gradient: 0-60% EtOAc in heptane) to provide 2,2,2-trifluoro-1-[(2′S,4S,7R)-4-hydroxy-2′-methyl-spiro[4,5-dihydrothieno[2,3-c]pyran-7,4′-piperidine]-1′-yl]ethenone (2.31 g, 83%). ¹H NMR (400 MHz, Chloroform-d) δ 7.23 (d, J=5.1 Hz, 1H), 7.00 (d, J=5.1 Hz, 1H), 4.55 (dt, J=9.2, 2.8 Hz, 1H), 4.48 (d, J=18.4 Hz, 1H), 3.96-3.79 (m, 3H), 3.49 (d, J=5.5 Hz, 1H), 2.39 (s, 1H), 2.29 (dd, J=14.5, 6.4 Hz, 1H), 1.99 (d, J=9.3 Hz, 1H), 1.97-1.88 (m, 1H), 1.88-1.77 (m, 1H), 1.32 (d, J=6.5 Hz, 3H). LCMS m/z 335.99 [M+H]⁺.

### Step 5. Synthesis of 1-[(2′S,4S,7R)-2-chloro-4-hydroxy-2′-methyl-spiro[4,5-dihydrothieno[2,3-c]pyran-7,4′-piperidine]-1′-yl]-2,2,2-trifluoro-ethanone (C46)

To a mixture of 2,2,2-trifluoro-1-[(2′S,4S,7R)-4-hydroxy-2′-methyl-spiro[4,5-dihydrothieno[2,3-c]pyran-7,4′-piperidine]-1′-yl]ethenone C45 (2.31 g, 6.89 mmol) in MeCN (30 mL) was added NCS (1200 mg, 8.987 mmol) and the mixture was heated to 70° C. under nitrogen. After 3 h, the reaction was cooled to rt, quenched with 1 M sodium bisulfite, and then diluted with EtOAc (50 mL) and water (30 mL). The organic layer was washed with brine (2×50 mL), dried with sodium sulfate and filtered. The organic layer was concentrated and purified by silica gel chromatography (Gradient: 0-50% EtOAc in heptane) to provide yield 1-[(2′S,4S,7R)-2-chloro-4-hydroxy-2′-methyl-spiro[4,5-dihydrothieno[2,3-c]pyran-7,4′-piperidine]-1′-yl]-2,2,2-trifluoro-ethanone (1.44 g, 47%). LCMS m/z 378.67 [M+H]⁺.

### Step 6. Synthesis of (2′S,4S,7R)-2-chloro-2′-methyl-spiro[4,5-dihydrothieno[2,3-c]pyran-7,4′-piperidine]-4-ol (S56)

To a mixture of 1-[(2′S,4S,7R)-2-chloro-4-hydroxy-2′-methyl-spiro[4,5-dihydrothieno[2,3-c]pyran-7,4′-piperidine]-1′-yl]-2,2,2-trifluoro-ethanone C45 (2700 mg, 7.302 mmol) in MeOH (26 mL) was added aqueous NaOH (10 mL of 2 M, 20.00 mmol). After 7 min, the mixture was concentrated to a minimal volume and quenched with HCl (20 mL of 1 M, 20.00 mmol), to a pH of about 10. At this time, the mixture was diluted with pH 10.5 buffer (50 mL) and DCM (100 mL). The layers were mixed and separated, and the aqueous layer was assayed and found to still have the product by UPLC. The mixture was pH adjusted with sodium hydroxide solution to pH 12, and then extracted with DCM (100 mL). The organic layer was combined with the first organic layer, dried with magnesium sulfate, filtered rinsed and passed over a phase separator to yield (2′S,4S,7R)-2-chloro-2′-methyl-spiro[4,5-dihydrothieno[2,3-c]pyran-7,4′-piperidine]-4-ol (1.93 g, 95%). ¹H NMR (400 MHz, DMSO-d₆) δ 6.94 (s, 1H), 5.35 (d, J=6.4 Hz, 1H), 4.35 (ddd, J=6.4, 5.2, 4.2 Hz, 1H), 3.86 (dd, J=11.8, 4.2 Hz, 1H), 3.58 (dd, J=11.8, 5.2 Hz, 1H), 2.85 (ddd, J=11.0, 6.3, 2.3 Hz, 1H), 2.78 (dd, J=12.3, 2.5 Hz, 1H), 2.73-2.63 (m, 1H), 1.97 (dt, J=13.3, 2.5 Hz, 1H), 1.92-1.76 (m, 2H), 1.45 (ddd, J=13.4, 12.3, 4.9 Hz, 1H), 1.15 (dd, J=13.3, 11.2 Hz, 1H), 0.94 (d, J=6.4 Hz, 3H). LCMS m/z 274.07 [M+H]⁺.

### Preparation S57

### (2′S, 7R)-2-chloro-2′-methyl-spiro[4,5-dihydrothieno[2,3-c]pyran-7,4′-piperidine]-4-ol (S57)

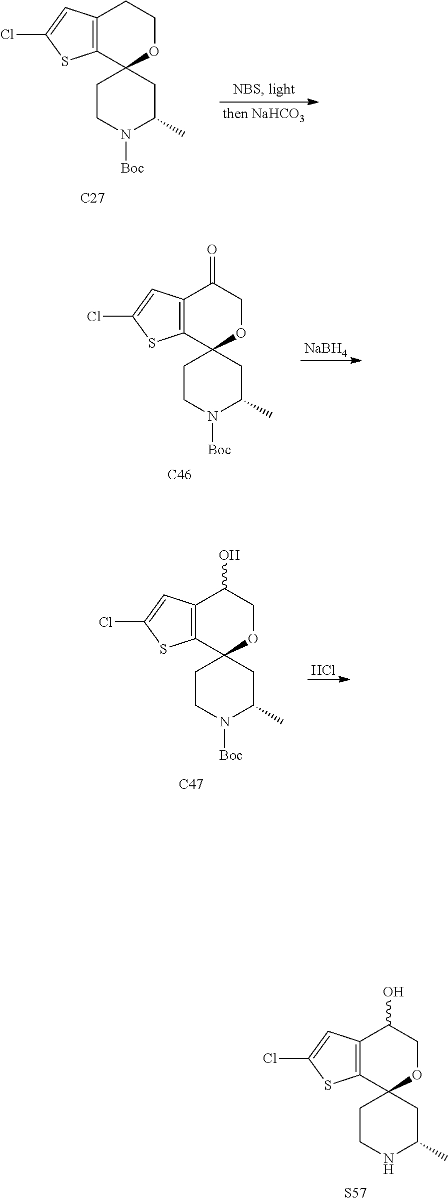

### Step 1. Synthesis of tert-butyl (2S,4R)-2′-chloro-2-methyl-4′-oxo-spiro[piperidine-4,7′-thieno[2,3-c]pyran]-1-carboxylate (C46)

Tert-butyl (2′S,7R)-2-chloro-2′-methyl-spiro[4,5-dihydrothieno[2,3-c]pyran-7,4′-piperidine]-1′-carboxylate C27 (3 g, 6.433 mmol) and NBS (1.67 g, 9.383 mmol) were dissolved in chlorobenzene (40 mL), and irradiated under a 100 W light bulb. After 30 h, the solution was quenched with aqueous sodium bisulfate solution, extracted with DCM, and filtered through a silica gel plug. The solvent was removed in vacuo. The resulting solid was dissolved in DMSO (30 mL) and NaHCO₃ (786 mg, 9.356 mmol) was added to the solution. The resulting mixture was heated to 50° C. and stirred overnight. Then the solution was quenched with H₂O and the solution was extracted with DCM (×3) and washed with brine. The product was purified by silica gel chromatography (Gradient: 0-20% EtOAc in hexanes) to provide tert-butyl (2S,4R)-2′-chloro-2-methyl-4′-oxo-spiro[piperidine-4,7′-thieno[2,3-c]pyran]-1-carboxylate (300 mg, 11%). LCMS m/z 371.83 [M+H]⁺.

### Step 2. Synthesis of tert-butyl (2S,4R)-2-chloro-4-hydroxy-2′-methyl-spiro[4,5-dihydrothieno[2,3-c]pyran-7,4′-piperidine]-1′-carboxylate (C47)

Dissolved tert-butyl (2S,4R)-2′-chloro-2-methyl-4′-oxo-spiro[piperidine-4,7′-thieno[2,3-c]pyran]-1-carboxylate C46 (2 g, 5.378 mmol) in DCM (80 mL) and MeOH (20 mL) under N₂, followed by addition of NaBH₄ (1.3 mL, 32.47 mmol). The reaction was stirred at room temperature for 1 h. It was then washed with water and extracted with DCM. The combined organic layer was washed with HCl, washed with brine and the solvent was removed in vacuo. The crude material was then purified by silica gel chromatography (Gradient: 0-40% EtOAc in hexanes) to provide tert-butyl (2S,4R)-2-chloro-4-hydroxy-2′-methyl-spiro[4,5-dihydrothieno[2,3-c]pyran-7,4′-piperidine]-1′-carboxylate (350 mg, 16%). ¹H NMR (300 MHz, Chloroform-d) δ 6.80 (d, J=5.4 Hz, 1H), 4.48-4.34 (m, 1H), 4.10-3.64 (m, 4H), 3.29 (ddt, J=14.1, 8.9, 5.4 Hz, 1H), 2.49-2.02 (m, 3H), 1.89-1.58 (m, 2H), 1.45 (s, 9H), 1.24 (dd, J=6.6, 3.6 Hz, 3H). LCMS m/z 374.26 [M+H]⁺.

### Step 3. Synthesis of (2′S, 7R)-2-chloro-2′-methyl-spiro[4,5-dihydrothieno[2,3-c]pyran-7,4′-piperidine]-4-ol (S57)

Tert-butyl (2S,4R)-2-chloro-4-hydroxy-2′-methyl-spiro[5,6-dihydro-4H-benzothiophene-7,4′-piperidine]-1′-carboxylate C47 (240 mg, 0.6453 mmol) was dissolved in dioxane (3.6 mL) and HCl (3.2 mL of 4 M, 12.80 mmol) was added. The solution was stirred overnight. The solvent was then removed in vacuo to provide (2′S,7R)-2-chloro-2′-methyl-spiro[4,5-dihydrothieno[2,3-c]pyran-7,4′-piperidine]-4-ol (176 mg, 100%). LCMS m/z 274.03 [M+H]⁺.

### Preparation S58

### (2S,4R)-2′-chloro-2-methyl-spiro[piperidine-4,7′-thieno[2,3-c]pyran]-4′-one (S58)

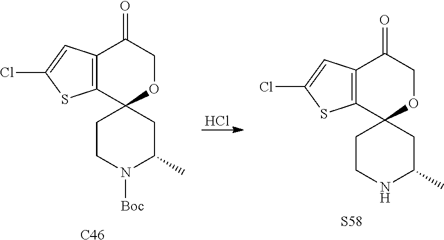 [SMILES: C[C@H]1C[C@@]2(CCN1C(=O)OC(C)(C)C)OCC(=O)c1cc(Cl)sc12 ; Cl ; C[C@H]1C[C@@]2(CCN1)OCC(=O)c1cc(Cl)sc12 ; "S58" ; "C46"]

Tert-butyl (2S,4R)-2′-chloro-2-methyl-4′-oxo-spiro[piperidine-4,7′-thieno[2,3-c]pyran]-1-carboxylate C46 (20 mg, 0.05378 mmol) was dissolved in dioxane (520 μL) with HCl (200 μL of 4 M, 0.8000 mmol). The solution was stirred at room temperature overnight. The solvent was removed in vacuo to give (2S,4R)-2′-chloro-2-methyl-spiro[piperidine-4,7′-thieno[2,3-c]pyran]-4′-one (Hydrochloride salt) (14.64 mg, 31%). LCMS m/z 272.38 [M+H]⁺.

### Compound 766

### (2′S,4R,7R)-2-chloro-1′-[(3-chloro-2-hydroxy-phenyl)methyl]-2′-methyl-spiro[4,5-dihydrothieno[2,3-c]pyran-7,4′-piperidine]-4-ol (766)

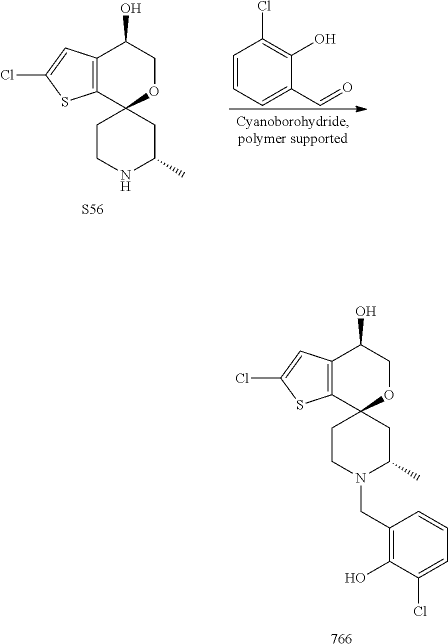 [SMILES: C[C@H]1C[C@@]2(CCN1)OC[C@H](O)c1cc(Cl)sc12 ; O=Cc1cccc(Cl)c1O ; "S56" ; C[C@H]1C[C@@]2(CCN1Cc1cccc(Cl)c1O)OC[C@H](O)c1cc(Cl)sc12 ; "766"]

Standard Method #A: Reductive Amination with Polymer Supported Cyanoborohydride

To a 1-dram vial containing of 3-chloro-2-hydroxy-benzaldehyde solution (400 μL, 0.25 M in DMF, 0.1 mmol) was charged with cyanoborohydride, polymer-supported (75 mg, 0.15 mmol, 2 mmol/g loading), followed by the addition of (2′S,4S,7R)-2-chloro-2′-methyl-spiro[4,5-dihydrothieno[2,3-c]pyran-7,4′-piperidine]-4-ol S56 (400 μL, 0.14 M in DMF, 0.056 mmol; with a catalytic amount of TFA). The vial was sealed and heated at 85° C. overnight. Then the vial was cooled to room temperature. The resulting suspension was filtered through a 25 micron polypropylene filter plate. TFA (400 μL, 0.1 wt % in water) was added to the reaction vial, which was stirred for several min to rinse the beads. The additional aqueous wash was passed through the filter plate. DMSO (200 μL) was added to the resulting filtrate. The resulting crude solution was filtered through a 0.45 micron syringe filter. Purification by reversed-phase HPLC. Method: C18 Waters Sunfire column (30×150 mm, 5 micron). Gradient: MeCN in H₂O with 0.1% trifluoroacetic acid. Product was isolated as (2′S,4R,7R)-2-chloro-1′-[(3-chloro-2-hydroxy-phenyl)methyl]-2′-methyl-spiro[4,5-dihydrothieno[2,3-c]pyran-7,4′-piperidine]-4-ol (trifluoroacetic acid salt) (4.5 mg, 15%). LCMS m/z 414.29 [M+H]⁺.

### Compounds 767-769

### (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (767, 768, 769)

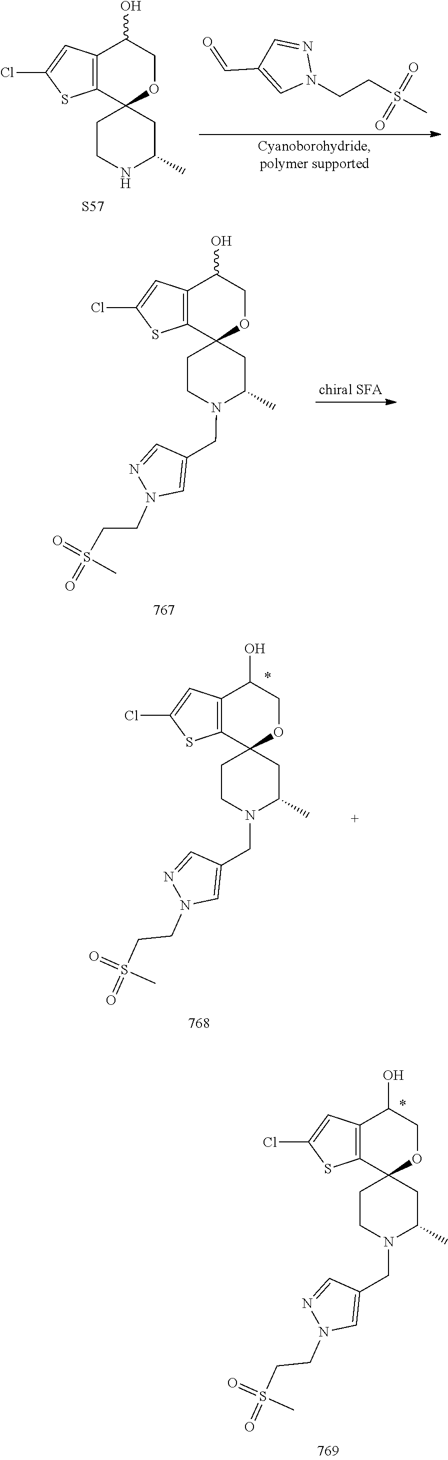

### Step 1. Synthesis of (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (767) —Standard Method #B: Reductive Amination with Polymer Supported Cyanoborohydride and Acetic Acid in Microwave Reactor

(2′S,7R)-2-chloro-2′-methyl-spiro[4,5-dihydrothieno[2,3-c]pyran-7,4′-piperidine]-4-ol S57 (109 mg, 0.3981 mmol) and 1-(2-methylsulfonylethyl)pyrazole-4-carbaldehyde (161 mg, 0.7961 mmol) were dissolved in DCM (2.94 mL), and acetic acid (120 mg, 1.998 mmol) was added to the solution. The solution was transferred to a microwave tube (5 mL) and cyanoborohydride, polymer-supported (594 mg of 2 mmol/g, 1.188 mmol) was added. The reaction mixture was capped and heated to 110° C. in a microwave reactor for 140 min. The solution was filtered and purified by silica gel chromatography (Gradient: 0-60% EtOAc in hexanes) to give (2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (90.9 mg, 49%). ¹H NMR (300 MHz, Methanol-d₄) δ 7.74 (s, 1H), 7.55 (s, 1H), 6.85 (d, J=0.7 Hz, 1H), 4.63 (t, J=6.4 Hz, 2H), 4.41 (dt, J=6.9, 3.8 Hz, 1H), 4.07-3.60 (m, 6H), 2.87-2.48 (m, 7H), 2.10 (t, J=9.6 Hz, 2H), 1.96-1.42 (m, 2H), 1.26 (dd, J=6.3, 4.1 Hz, 3H). LCMS m/z 460.11 [M+H]⁺.

### Step 2. Synthesis of (2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (768 and 769)

(2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol 767 was separated into constituent enantiomers by chiral SFC separation. Column: Daicel Chiralpak® IC, 10×250 mm; Mobile Phase: 40% MeOH (containing 5 mM ammonia), 60% carbon dioxide. Flow: 15 mL/min. Two products were obtained:

(2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (24.2 mg, 26%). ¹H NMR (400 MHz, Methanol-d₄) δ 8.00 (s, 1H), 7.73 (d, J=0.7 Hz, 1H), 6.92 (s, 1H), 4.70 (dd, J=6.9, 5.4 Hz, 2H), 4.54 (d, J=14.1 Hz, 1H), 4.45 (t, J=3.5 Hz, 1H), 4.28 (d, J=14.1 Hz, 1H), 3.94 (dd, J=12.3, 3.4 Hz, 1H), 3.81-3.69 (m, 3H), 3.57 (dd, J=10.4, 6.4 Hz, 1H), 3.40-3.21 (m, 2H), 2.87 (s, 3H), 2.37 (ddd, J=21.0, 15.0, 3.0 Hz, 2H), 2.03-1.82 (m, 2H), 1.54 (d, J=6.5 Hz, 3H). LCMS m/z 460.06 [M+H]⁺; and

(2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (23.6 mg, 25%). ¹H NMR (400 MHz, Methanol-d₄) δ 7.74 (s, 1H), 7.54 (d, J=0.8 Hz, 1H), 6.85 (s, 1H), 4.63 (t, J=6.4 Hz, 2H), 4.42 (t, J=4.0 Hz, 1H), 3.99-3.56 (m, 6H), 2.88-2.50 (m, 6H), 2.20-1.73 (m, 3H), 1.53 (dd, J=14.3, 11.6 Hz, 1H), 1.24 (d, J=6.3 Hz, 3H). LCMS m/z 460.11 [M+H]⁺.

### Compounds 770-812

Compounds 770-812 (see Table 33) were prepared in a single step from intermediate S56 and S57 using methods described for compounds 766-769. Aldehydes were obtained from commercial sources or described previously. Any modifications to methods are noted in Table 33 and accompanying footnotes.

TABLE 33
Structure and physicochemical data for compounds 770-812

| | | | Starting | ¹H NMR; |
|---|---|---|---|---|
| | | | Material, | LCMS |
| | | Aldehyde | Standard | m/z |
| Cmpd | Structure | Reagent | Method | [M + H]⁺ |
| 770 | 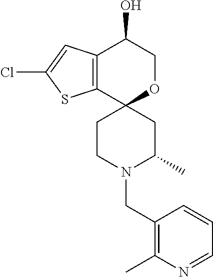 [SMILES: Cc1ncccc1CN1CC[C@]2(C[C@@H]1C)OC[C@H](O)c1cc(Cl)sc12] | 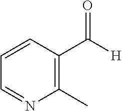 | S56, Compound 766 | LCMS m/z 379.34 [M + H]⁺. |
| 771 | 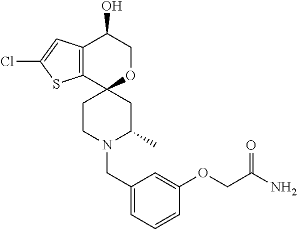 [SMILES: C[C@H]1C[C@@]2(CCN1Cc1cccc(OCC(N)=O)c1)OC[C@H](O)c1cc(Cl)sc12] | 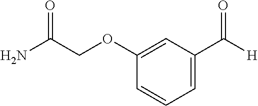 [SMILES: NC(=O)COc1cccc(C=O)c1] | S56, Compound 766 | LCMS m/z 437.36 [M + H]⁺. |
| 772 | 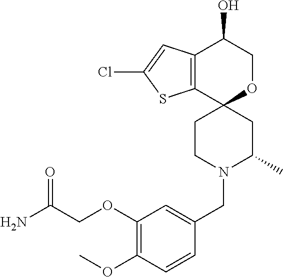 [SMILES: COc1ccc(CN2CC[C@]3(C[C@@H]2C)OC[C@H](O)c2cc(Cl)sc23)cc1OCC(N)=O] | 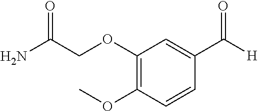 | S56, Compound 766 | LCMS m/z 467.39 [M + H]⁺. |
| 773 | 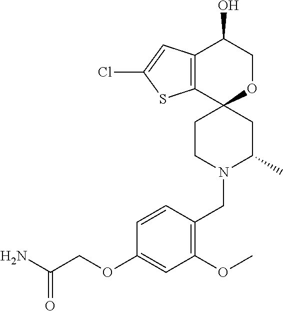 [SMILES: COc1cc(OCC(N)=O)ccc1CN1CC[C@]2(C[C@@H]1C)OC[C@H](O)c1cc(Cl)sc12] | 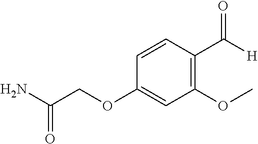 | S56, Compound 766 | LCMS m/z 467.39 [M + H]⁺. |
| 774 | 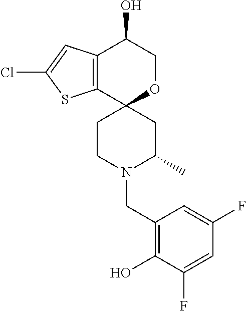 [SMILES: C[C@H]1C[C@@]2(CCN1Cc1cc(F)cc(F)c1O)OC[C@H](O)c1cc(Cl)sc12] | 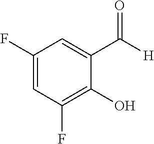 [SMILES: O=Cc1cc(F)cc(F)c1O] | S56, Compound 766 | LCMS m/z 416.28 [M + H]⁺. |
| 775 | 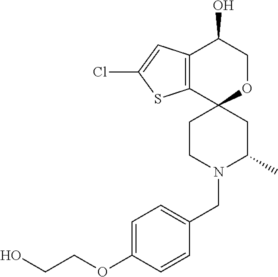 [SMILES: C[C@H]1C[C@@]2(CCN1Cc1ccc(OCCO)cc1)OC[C@H](O)c1cc(Cl)sc12] | 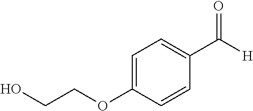 [SMILES: O=Cc1ccc(OCCO)cc1] | S56, Compound 766 | LCMS m/z 424.36 [M + H]⁺. |
| 776 | 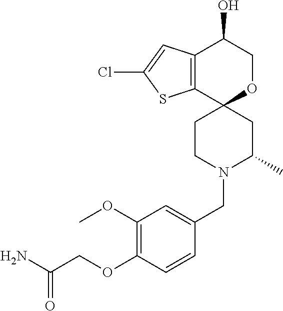 [SMILES: COc1cc(CN2CC[C@]3(C[C@@H]2C)OC[C@H](O)c2cc(Cl)sc23)ccc1OCC(N)=O] | 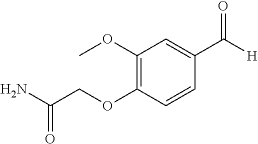 | S56, Compound 766 | LCMS m/z 467.39 [M + H]⁺. |
| 777 | 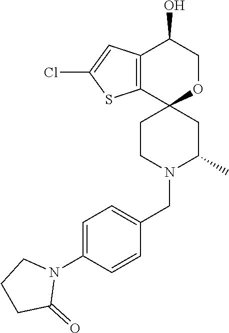 [SMILES: C[C@H]1C[C@@]2(CCN1Cc1ccc(N3CCCC3=O)cc1)OC[C@H](O)c1cc(Cl)sc12] | 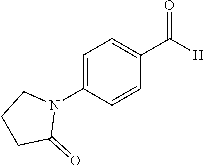 [SMILES: O=Cc1ccc(N2CCCC2=O)cc1] | S56, Compound 766 | LCMS m/z 447.38 [M + H]⁺. |
| 778 | 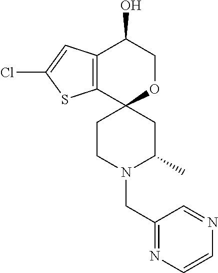 [SMILES: C[C@H]1C[C@@]2(CCN1Cc1cnccn1)OC[C@H](O)c1cc(Cl)sc12] | 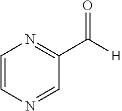 | S56, Compound 766 | LCMS m/z 366.29 [M + H]⁺. |
| 779 | 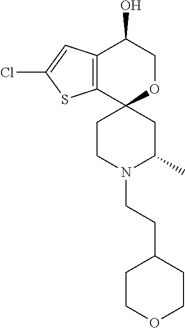 [SMILES: C[C@H]1C[C@@]2(CCN1CCC1CCOCC1)OC[C@H](O)c1cc(Cl)sc12] | 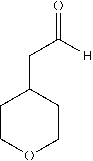 | S56, Compound 766 | LCMS m/z 386.38 [M + H]⁺. |
| 780 | 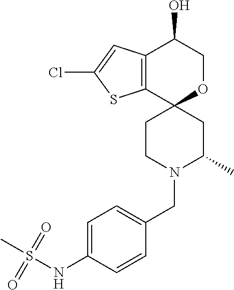 | 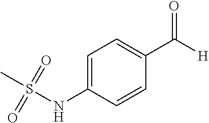 | S56, Compound 766 | LCMS m/z 457.36 [M + H]⁺. |
| 781 | 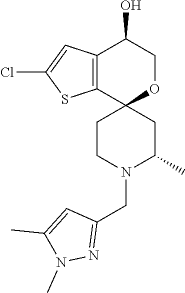 | 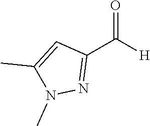 | S56, Compound 766 | LCMS m/z 382.36 [M + H]⁺. |
| 782 | 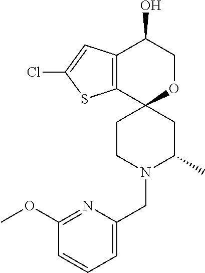 | 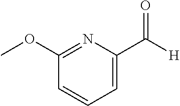 | S56, Compound 766 | LCMS m/z 395.32 [M + H]⁺. |
| 783 | 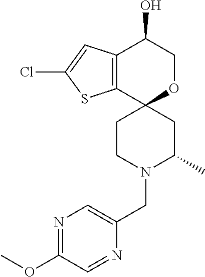 | 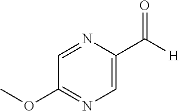 | S56, Compound 766 | LCMS m/z 396.32 [M + H]⁺. |
| 784 | 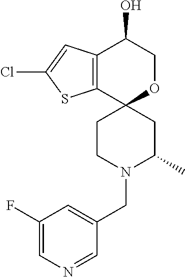 [SMILES: C[C@H]1C[C@@]2(CCN1Cc1cncc(F)c1)OC[C@H](O)c1cc(Cl)sc12] | 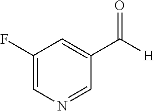 | S56, Compound 766 | LCMS m/z 383.31 [M + H]⁺. |
| 785 | 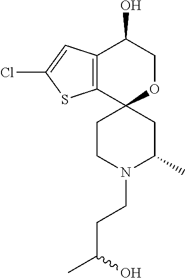 [SMILES: CC(O)CCN1CC[C@]2(C[C@@H]1C)OC[C@H](O)c1cc(Cl)sc12] | 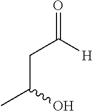 | S56, Compound 766 | LCMS m/z 346.33 [M + H]⁺. |
| 786 | 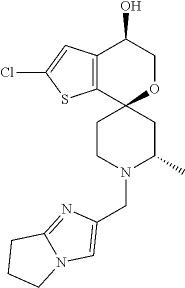 [SMILES: C[C@H]1C[C@@]2(CCN1Cc1cn3c(n1)CCC3)OC[C@H](O)c1cc(Cl)sc12] | 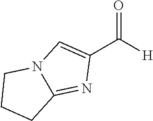 | S56, Compound 766 | LCMS m/z 394.33 [M + H]⁺. |
| 787 | 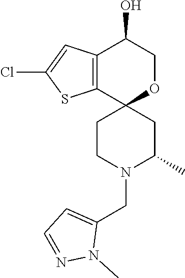 [SMILES: C[C@H]1C[C@@]2(CCN1Cc1ccnn1C)OC[C@H](O)c1cc(Cl)sc12] | 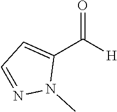 | S56, Compound 766 | LCMS m/z 368.32 [M + H]⁺. |
| 788 | 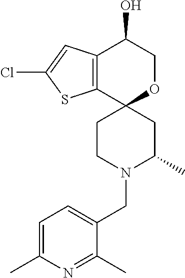 | 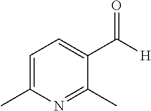 | S56, Compound 766 | LCMS m/z 393.34 [M + H]⁺. |
| 789 | 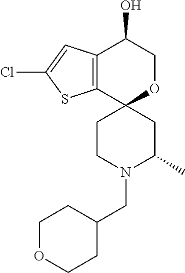 [SMILES: C[C@H]1C[C@@]2(CCN1CC1CCOCC1)OC[C@H](O)c1cc(Cl)sc12] | 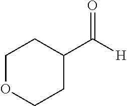 | S56, Compound 766 | LCMS m/z 372.34 [M + H]⁺. |
| 790 | 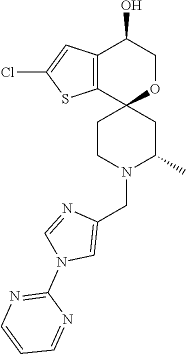 [SMILES: C[C@H]1C[C@@]2(CCN1Cc1cn(-c3ncccn3)cn1)OC[C@H](O)c1cc(Cl)sc12] | 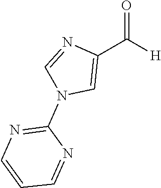 [SMILES: O=Cc1cn(-c2ncccn2)cn1] | S56, Compound 766 | LCMS m/z 432.35 [M + H]⁺. |
| 791 | 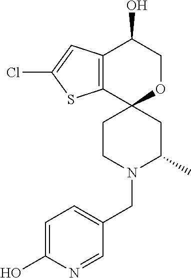 [SMILES: C[C@H]1C[C@@]2(CCN1Cc1ccc(O)nc1)OC[C@H](O)c1cc(Cl)sc12] | 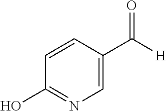 | S56, Compound 766 | LCMS m/z 381.33 [M + H]⁺. |
| 792 | 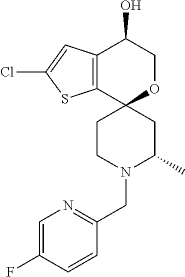 [SMILES: C[C@H]1C[C@@]2(CCN1Cc1ccc(F)cn1)OC[C@H](O)c1cc(Cl)sc12] | 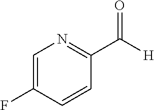 | S56, Compound 766 | LCMS m/z 383.31 [M + H]⁺. |
| 793 | 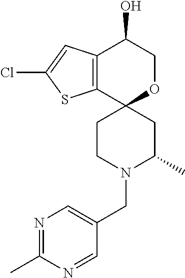 | 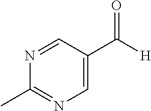 | S56, Compound 766 | LCMS m/z 380.33 [M + H]⁺. |
| 794 | 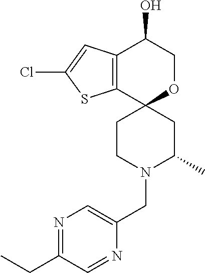 [SMILES: CCc1cnc(CN2CC[C@]3(C[C@@H]2C)OC[C@H](O)c2cc(Cl)sc23)cn1] | 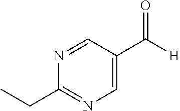 | S56, Compound 766 | LCMS m/z 394.33 [M + H]⁺. |
| 795 | 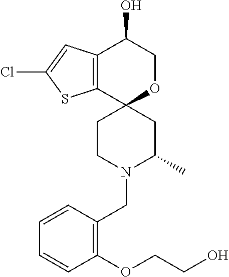 [SMILES: C[C@H]1C[C@@]2(CCN1Cc1ccccc1OCCO)OC[C@H](O)c1cc(Cl)sc12] | 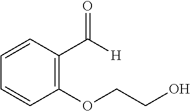 [SMILES: O=Cc1ccccc1OCCO] | S56, Compound 766 | LCMS m/z 424.36 [M + H]⁺. |
| 796 | 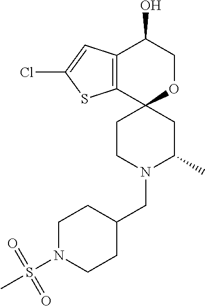 | 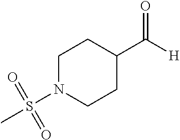 | S56, Compound 766 | LCMS m/z 449.37 [M + H]⁺. |
| 797 | 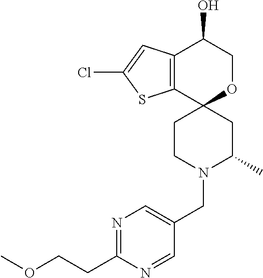 | 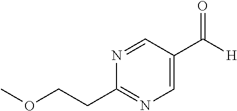 | S56, Compound 766 | LCMS m/z 424.36 [M + H]⁺. |
| 798 | 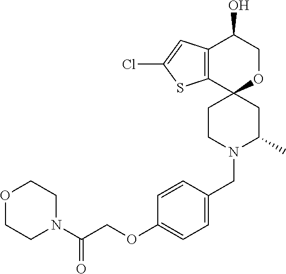 [SMILES: C[C@H]1C[C@@]2(CCN1Cc1ccc(OCC(=O)N3CCOCC3)cc1)OC[C@H](O)c1cc(Cl)sc12] | 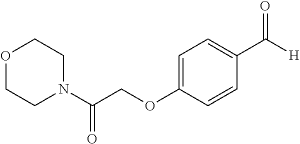 [SMILES: O=Cc1ccc(OCC(=O)N2CCOCC2)cc1] | S56, Compound 766 | LCMS m/z 507.43 [M + H]⁺. |
| 799 | 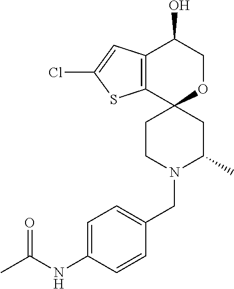 [SMILES: CC(=O)Nc1ccc(CN2CC[C@]3(C[C@@H]2C)OC[C@H](O)c2cc(Cl)sc23)cc1] | 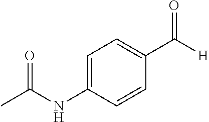 | S56, Compound 766 | LCMS m/z 421.33 [M + H]⁺. |
| 800 | 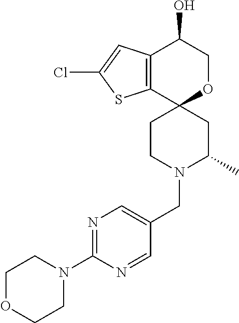 [SMILES: C[C@H]1C[C@@]2(CCN1Cc1cnc(N3CCOCC3)nc1)OC[C@H](O)c1cc(Cl)sc12] | 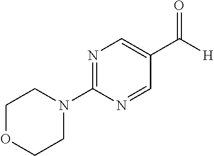 [SMILES: O=Cc1cnc(N2CCOCC2)nc1] | S56, Compound 766 | LCMS m/z 451.36 [M + H]⁺. |
| 801 | 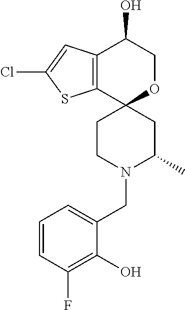 [SMILES: C[C@H]1C[C@@]2(CCN1Cc1cccc(F)c1O)OC[C@H](O)c1cc(Cl)sc12] | 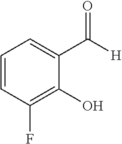 | S56, Compound 766 | LCMS m/z 398.31 [M + H]⁺. |
| 802 | 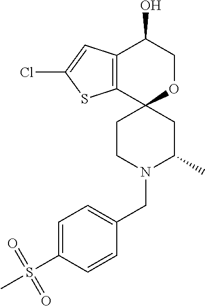 | 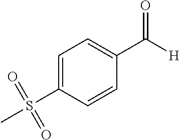 | S56, Compound 766 | LCMS m/z 442.28 [M + H]⁺. |
| 803 | 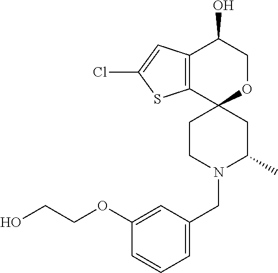 [SMILES: C[C@H]1C[C@@]2(CCN1Cc1cccc(OCCO)c1)OC[C@H](O)c1cc(Cl)sc12] | 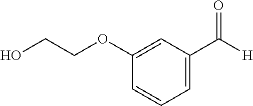 [SMILES: O=Cc1cccc(OCCO)c1] | S56, Compound 766 | LCMS m/z 424.31 [M + H]⁺. |
| 804 | 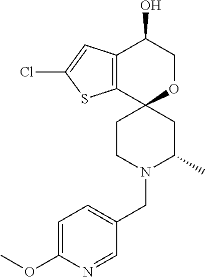 | 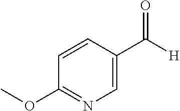 | S56, Compound 766 | LCMS m/z 395.37 [M + H]⁺. |
| 805 | 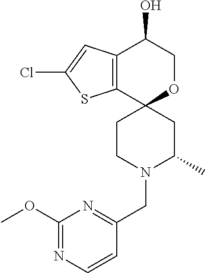 | 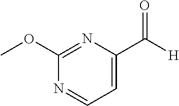 | S56, Compound 766 | LCMS m/z 396.32 [M + H]⁺. |
| 806 | 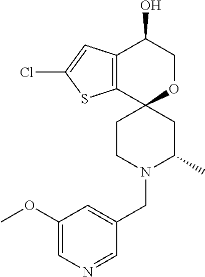 | 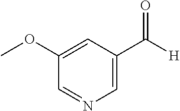 | S56, Compound 766 | LCMS m/z 395.32 [M + H]⁺. |
| 807 | 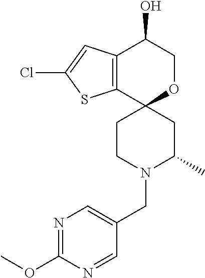 | 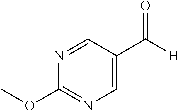 | S56, Compound 766 | LCMS m/z 396.32 [M + H]⁺. |
| 808 | 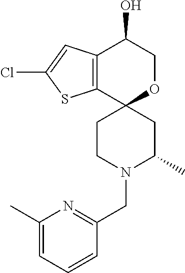 | 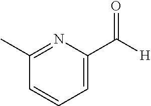 | S56, Compound 766 | LCMS m/z 379.3 [M + H]⁺. |
| 809 | 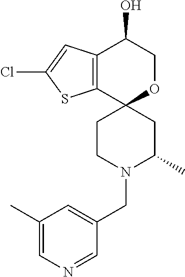 | 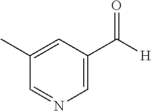 | S56, Compound 766 | LCMS m/z 379.34 [M + H]⁺. |
| 810 | 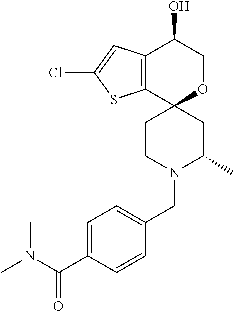 | 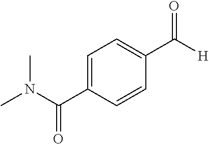 | S56, Compound 766 | LCMS m/z 435.37 [M + H]⁺. |
| 811 | 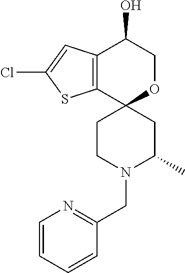 [SMILES: C[C@H]1C[C@@]2(CCN1Cc1ccccn1)OC[C@H](O)c1cc(Cl)sc12] | 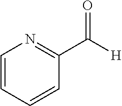 | S56, Compound 766 | LCMS m/z 365.34 [M + H]⁺. |
| 812 | 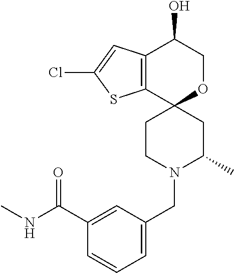 | 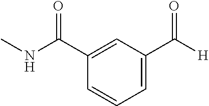 | S56, Compound 766 | ¹H NMR (400 MHz, DMSO-d₆) δ 9.62 (d, J = 10.6 Hz, 1H), 8.55 (d, J = 4.8 Hz, 1H), 7.99 (s, 1H), 7.91 (d, J = 7.8 Hz, 1H), 7.66 (d, J = 7.7 Hz, 1H), 7.57 (t, J = 7.7 Hz, 1H), 7.01 (d, J = 3.5 Hz, 1H), 5.49 (s, 1H), 4.81 (d, J = 12.8 Hz, 1H), 4.38 (d, J = 4.4 Hz, 1H), 4.19 (dd, J = 12.8, 8.3 Hz, 1H), 3.89 (dd, J = |
| | | | | 12.0, 3.9 Hz, 1H), |
| | | | | 3.67 (dd, J = 12.0, |
| | | | | 4.7 Hz, 1H), 3.59 |
| | | | | (s, 1H), 3.18-3.09 |
| | | | | (m, 1H), 2.98- |
| | | | | 2.88 (m, 1H), 2.80 |
| | | | | (d, J = 4.4 Hz, 3H), |
| | | | | 2.44-2.07 (m, |
| | | | | 2H), 1.88 (dt, J = |
| | | | | 50.4, 13.6 Hz, 2H), |
| | | | | 1.51 (d, J = 6.4 Hz, |
| | | | | 3H); LCMS m/z |
| | | | | 421.33 [M + H]⁺. |
| 813 | 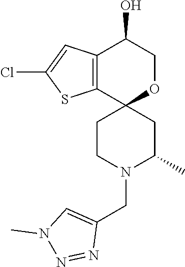 | 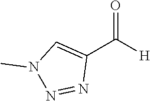 | S56, Compound 766 | ¹H NMR (400 MHz, DMSO-d₆) δ 10.02 (s, 1H), 8.30 (s, 1H), 7.01 (s, 1H), 5.43 (s, 1H), 4.62 (d, J = 14.3 Hz, 1H), 4.50 (d, J = 13.9 Hz, 1H), 4.37 (s, 1H), 4.11 (s, 3H), 3.85 (dd, J = 11.8, 4.0 Hz, 1H), 3.61 (dd, J = 12.0, 4.8 Hz, 1H), 3.38-3.26 (m, 2H), 3.23-3.08 (m, 1H), 2.26 (dd, J = 55.6, 14.7 Hz, |
| | | | | 2H), 1.92 (t, J = |
| | | | | 13.4 Hz, 2H), 1.46 |
| | | | | (d, J = 6.4 Hz, |
| | | | | 3H); LCMS m/z |
| | | | | 369.36 [M + H]⁺. |
| 814 | 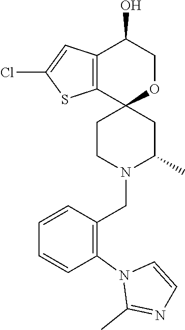 [SMILES: Cc1nccn1-c1ccccc1CN1CC[C@]2(C[C@@H]1C)OC[C@H](O)c1cc(Cl)sc12] | 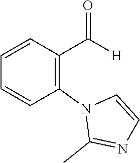 | S56, Compound 766 | LCMS m/z 444.36 [M + H]⁺. |
| 815 | 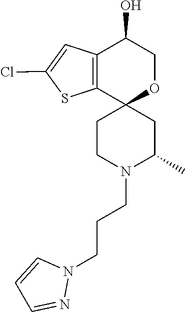 [SMILES: C[C@H]1C[C@@]2(CCN1CCCn1cccn1)OC[C@H](O)c1cc(Cl)sc12] | 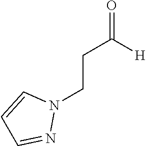 | S56, Compound 766 | ¹H NMR (400 MHz, DMSO-d₆) δ 9.51 (s, 1H), 7.78 (d, J = 2.2 Hz, 1H), 7.49 (d, J = 1.8 Hz, 1H), 7.02 (s, 1H), 6.28 (t, J = 2.0 Hz, 1H), 5.48 (s, 1H), 4.43-4.36 (m, 1H), 4.23 (t, J = 6.7 Hz, 2H), 3.91 (dd, J = 12.0, 4.0 Hz, 1H), 3.66 (dd, J = 12.0, 4.9 Hz, 1H), 3.37-3.21 (m, 2H), 3.16 (q, J = 11.2 Hz, 1H), 3.09-2.98 (m, 1H), 2.39-2.01 |
| | | | | (m, 4H), 2.03- |
| | | | | 1.75 (m, 2H), 1.21 |
| | | | | (d, J = 6.3 Hz, |
| | | | | 3H); LCMS m/z |
| | | | | 382.36 [M + H]⁺. |
| 816 | 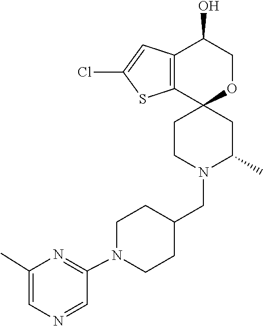 | 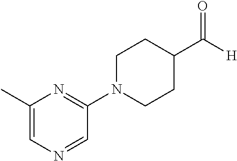 | S56, Compound 766 | ¹H NMR (400 MHz, DMSO-d₆) δ 8.83 (s, 1H), 8.14 (s, 1H), 7.70 (s, 1H), 7.03 (s, 1H), 4.47-4.32 (m, 4H), 3.93 (dd, J = 12.0, 4.0 Hz, 1H), 3.68 (dd, J = 11.8, 4.9 Hz, 1H), 3.56 (d, J = 12.3 Hz, 1H), 3.51-3.43 (m, 1H), 3.21 (dt, J = 55.8, 11.3 Hz, 2H), 3.03-2.80 (m, 4H), 2.30 (d, J = 22.5 Hz, 1H), 2.29 (s, 3H), 2.23- 1.93 (m, 3H), 1.94- 1.64 (m, 2H), |
| | | | | 1.31 (d, J = 6.3 Hz, |
| | | | | 3H), 1.29-1.12 |
| | | | | (m, 2H); LCMS |
| | | | | m/z 463.41 |
| | | | | [M + H]⁺. |
| 817 | 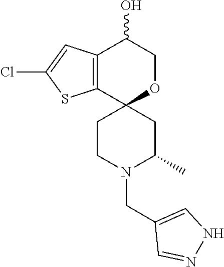 | 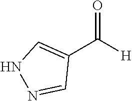 | S57, Compound 767¹ | ¹H NMR (400 MHz, Chloroform- d) δ 7.51 (s, 2H), 6.80 (d, J = 2.0 Hz, 1H), 4.39 (dt, J = 12.2, 2.8 Hz, 1H), 3.87 (ddt, J = 26.6, 14.4, 11.0 Hz, 3H), 3.58 (dd, J = 14.2, 5.7 Hz, 1H), 2.78- 2.38 (m, 3H), 2.12- 1.99 (m, 1H), 1.97-1.46 (m, 3H), 1.21 (t, J = 3.3 Hz, 3H); LCMS m/z 353.95 [M + H]⁺. |
| 818 | 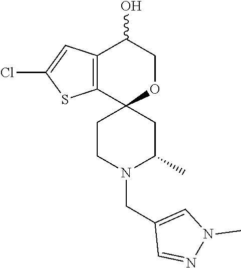 | 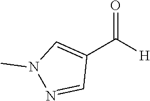 | S57, Compound 767² | ¹H NMR (300 MHz, Methanol- d₄) δ 7.88 (s, 1H), 7.66 (d, J = 0.8 Hz, 1H), 6.91 (s, 1H), 4.60-4.39 (m, 2H), 4.26 (d, J = 14.1 Hz, 1H), 4.05- 3.89 (m, 4H), 3.76 (ddd, J = 12.2, 9.3, 3.9 Hz, 1H), 3.69-3.41 (m, 1H), 3.44- 3.32 (m, 1H), 2.49- 2.26 (m, 2H), 2.16-1.77 (m, 2H), 1.54 (dd, J = |
| | | | | 6.5, 3.6 Hz, 3H) |
| | | | | some overlap with |
| | | | | solvent peak; |
| | | | | LCMS m/z 368.15 |
| | | | | [M + H]⁺. |
| 819 | 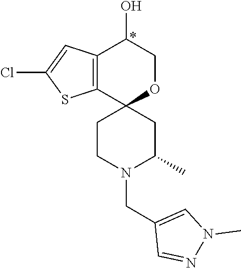 | 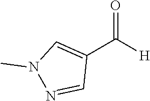 | S57, Compound 768-769²³ | ¹H NMR (300 MHz, Methanol- d₄) δ 7.57 (s, 1H), 7.42 (d, J = 0.8 Hz, 1H), 6.84 (s, 1H), 4.39 (t, J = 3.7 Hz, 1H), 3.99-3.47 (m, 7H), 2.76- 2.42 (m, 3H), 2.15- 1.98 (m, 2H), 1.81-1.51 (m, 2H), 1.23 (d, J = 6.3 Hz, 3H). |
| 820 | 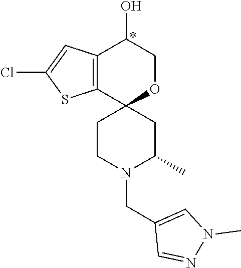 | 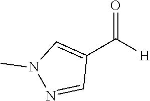 | S57, Compound 768-769³ | ¹H NMR (300 MHz, Methanol- d₄) δ 7.57 (s, 1H), 7.43 (d, J = 0.8 Hz, 1H), 6.85 (s, 1H), 4.41 (t, J = 4.1 Hz, 1H), 4.00-3.55 (m, 7H), 2.88- 2.38 (m, 3H), 2.15- 1.97 (m, 2H), 1.82 (td, J = 13.3, 4.6 Hz, 1H), 1.51 (dd, J = 14.1, 11.5 Hz, 1H), 1.21 (d, J = 6.2 Hz, 3H). |

Footnotes:
¹Different reagent amounts were used: 1H-pyrazole-4-carbaldehyde (1.1 eq), acetic acid (2.6 eq). Purification was performed with reverse phase HPLC with C18 column (Gradient: 0-60% MeCN in water, with TFA modifier).
²Different reagent amounts were used: 1-methylpyrazole-4-carbaldehyde (1.5 eq). Purification by reversed-phase HPLC. Method: C18 Waters Sunfire column (30 × 150 mm, 5 micron). Gradient: MeCN in H₂O with 0.1% trifluoroacetic acid.
³The racemic mixture 818 was separated into constituent enantiomers by chiral SFC separation. Column: Daicel Chiralpak ® IC, 10 × 250 mm; Mobile Phase: 40% MeOH (containing 5 mM ammonia), 60% carbon dioxide. Flow: 15 mL/min.

### Compound 821

### (2′S,4S,7R)-2-chloro-1′-ethyl-2′-methyl-spiro[4,5-dihydrothieno[2,3-c]pyran-7,4′-piperidine]-4-ol (821)

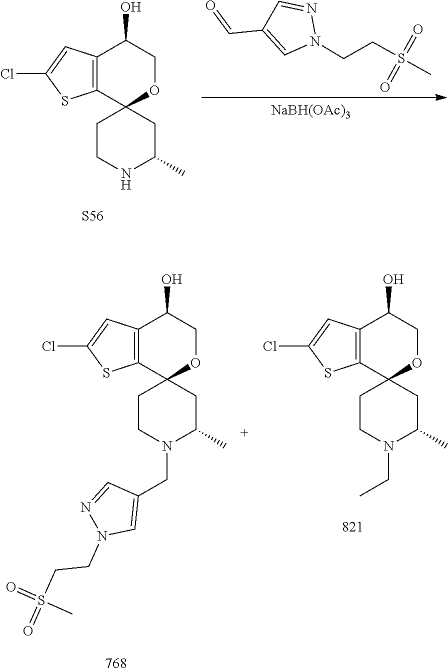

To a suspension of (2′S,4S,7R)-2-chloro-2′-methyl-spiro[4,5-dihydrothieno[2,3-c]pyran-7,4′-piperidine]-4-ol S56 (48 g, 175.3 mmol) and 1-(2-methylsulfonylethyl)pyrazole-4-carbaldehyde (36.5 g, 180.5 mmol) in THF (750 mL) at rt was added sodium triacetoxyborohydride (73 g, 344.4 mmol). The reaction mixture was heated to 55° C. After 2 h, additional 1-(2-methylsulfonylethyl)pyrazole-4-carbaldehyde (1.0 g, 4.945 mmol) was added. After 1 h, the reaction was cooled to room temperature using an ice-water bath, and then slowly quenched with saturated aqueous sodium bicarbonate (1.3 L). The bisphasic mixture was treated with potassium carbonate (29 g), then ice-water bath was removed. The mixture was treated with EtOAc (300 mL) and stirred at room temperature overnight. After 16 h, layers were separated. The organic layer was washed with brine (800 mL). The aqueous layer was extracted with EtOAc (1 L), and the extract was washed with brine (500 mL), then combined with original organic layer. The combined organic layer was dried with MgSO₄, filtered and concentrated. Purification by silica gel chromatography (Gradient: 0-100% EtOAc in heptane, with 5% Et₂NH modifier) to afford desired product mixed impurities. It was further purified by SFC. Column: PYR, 4.6×150 mm; Mobile Phase: 20% MeOH (containing 0.2% Et₂NH), 80% carbon dioxide. Flow: 5 mL/min. Two products were isolated: major product is (2′S,4S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol (76 g, 94%) as yellow foam. And the minor peak was identified as (2′S,4S,7R)-2-chloro-1′-ethyl-2′-methyl-spiro[4,5-dihydrothieno[2,3-c]pyran-7,4′-piperidine]-4-ol (2.9 g, 5%) as a brown oil. ¹H NMR (400 MHz, Chloroform-d) δ 6.80 (s, 1H), 4.40 (t, J=2.8 Hz, 1H), 4.00-3.83 (m, 2H), 2.89 (dq, J=13.2, 7.3 Hz, 1H), 2.81-2.63 (m, 2H), 2.58-2.47 (m, 2H), 2.11 (dq, J=14.3, 2.8 Hz, 1H), 1.94 (dt, J=13.7, 2.9 Hz, 1H), 1.75 (ddd, J=14.4, 13.0, 4.6 Hz, 1H), 1.67 (dd, J=13.8, 11.4 Hz, 1H), 1.09 (d, J=6.3 Hz, 3H), 1.04 (t, J=7.2 Hz, 3H). LCMS m/z 301.98 [M+H]⁺.

### Compound 822

### (2S,4R)-2′-chloro-2-methyl-1-[[1-(2-methylsulfonylethyl)triazol-4-yl]methyl]spiro[piperidine-4,7′-thieno[2,3-c]pyran]-4′-one (822)

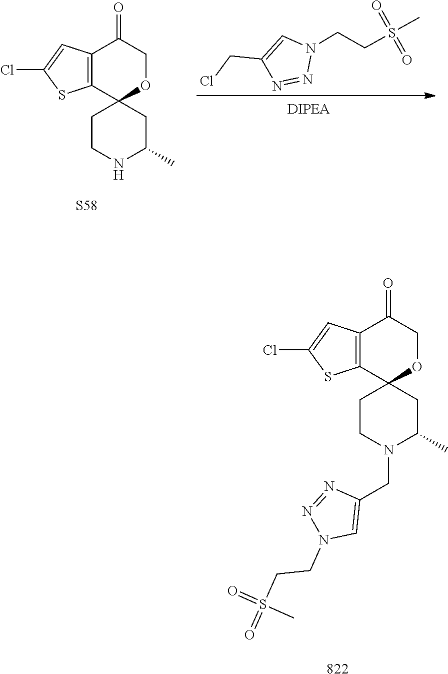 [SMILES: C[C@H]1C[C@@]2(CCN1)OCC(=O)c1cc(Cl)sc12 ; CS(=O)(=O)CCn1cc(CCl)nn1 ; "S58" ; C[C@H]1C[C@@]2(CCN1Cc1cn(CCS(C)(=O)=O)nn1)OCC(=O)c1cc(Cl)sc12 ; "822"]

### Standard Method #C: SN2 Alkylation

4-(Chloromethyl)-1-(2-methylsulfonylethyl)triazole (9 mg, 0.04024 mmol) and (2S,4R)-2′-chloro-2-methyl-spiro[piperidine-4,7′-thieno[2,3-c]pyran]-4′-one S58 (11 mg, 0.04048 mmol) were dissolved in DCE (300 μL) and DIPEA (15.7 mg, 0.1215 mmol) was added in a microwave vial. The vial was capped and the reaction mixture was heated to 65° C. and stirred for 24 h. The solution was quenched with MeOH and purified by reversed-phase HPLC. Method: C18 Waters Sunfire column (30×150 mm, 5 micron). Gradient: MeCN in H2O with 0.1% trifluoroacetic acid. The product was isolated as (2S,4R)-2′-chloro-2-methyl-1-[[1-(2-methylsulfonylethyl)triazol-4-yl]methyl]spiro[piperidine-4,7′-thieno[2,3-c]pyran]-4′-one (trifluoroacetate salt) (4.3 mg, 16%). ¹H NMR (300 MHz, Methanol-M₄) δ 8.35 (s, 1H), 7.26 (s, 1H), 5.10-4.94 (m, 2H), 4.85-4.56 (m, 2H), 4.49-4.31 (m, 2H), 4.08-3.76 (m, 2H), 3.65-3.45 (m, 1H), 3.42 (dd, J=13.9, 11.2 Hz, 2H), 3.01 (s, 3H), 2.68-2.45 (m, 2H), 2.26-1.92 (m, 2H), 1.61 (d, J=6.5 Hz, 3H).

### Compounds 823-824

Compounds 823-824 (see Table 34) were prepared in a single step from intermediate S58 using standard method #C. Alkyl halides were obtained from commercial sources or described previously. Any modifications to methods are noted in Table 34 and accompanying footnotes.

TABLE 34
Structure and physicochemical data for compounds 823-824

| | | Aldehyde | Starting | ¹H NMR; LCMS |
|---|---|---|---|---|
| Cmpd | Product | Reagent | Material | m/z [M + H]⁺ |
| 823 | 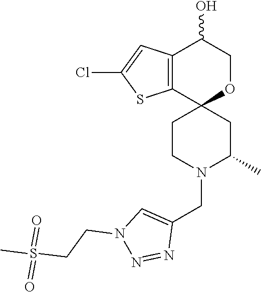 | 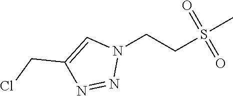 | S58 | ¹H NMR (300 MHz, Methanol-d₄) δ 8.32 (s, 1H), 6.91 (s, 1H), 5.11- 4.94 (m, 2H), 4.72-4.33 (m, 3H), 4.03-3.66 (m, 4H), 3.38 (s, 1H), 2.99 (d, J = 0.6 Hz, 3H), 2.43-1.80 (m, 4H), 1.54 (d, J = 6.4 Hz, 3H). some peaks overlapped with solvent peaks; LCMS m/z 461.38 [M + H]⁺. |
| 824 | 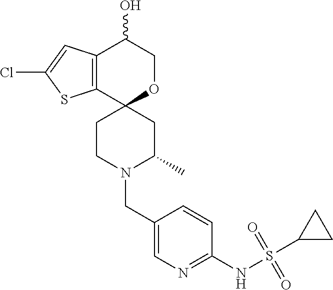 | 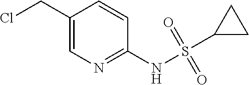 [SMILES: O=S(=O)(Nc1ccc(CCl)cn1)C1CC1] | S58¹ | ¹H NMR (300 MHz, Methanol-d₄) δ 8.09 (d, J = 2.3 Hz, 1H), 7.74 (dd, J = 8.6, 2.4 Hz, 1H), 7.16 (dt, J = 8.6, 0.9 Hz, 1H), 6.85 (d, J = 0.9 Hz, 1H), 4.42 (dt, J = 9.3, 3.9 Hz, 1H), 4.08 (d, J = 13.5 Hz, 1H), 3.95 (ddd, J = 12.5, 9.0, 3.7 Hz, 1H), 3.84-3.65 (m, 1H), 3.22 (d, J = 13.5 Hz, 1H), 2.91 (td, J = 8.1, 4.1 Hz, 1H), 2.86-2.55 (m, 2H), 2.55-2.34 (m, 1H), 2.20-1.96 (m, 2H), 1.87- 1.49 (m, 2H), 1.27-0.95 (m, 8H); LCMS m/z 484.01 [M + H]⁺. |

Footnotes:
¹(2′S,7R)-2-chloro-2′-methyl-spiro[4,5-dihydrothieno[2,3-c]pyran-7,4′-piperidine]-4-ol (1.5 eq) and N-[5-(chloromethyl)-2-pyridyl]cyclopropanesulfonamide (1.0 eq) was used. The reaction was completed in 2 h at 60° C. Purification was performed using silica gel chromatography (Gradient: 1-20% MeOH in DCM).

### Compound 825

### (2′S, 7R)-2-chloro-1′-[[2-[(4-hydroxycyclohexyl)amino]pyrimidin-5-yl]methyl]-2′-methyl-spiro[4,5-dihydrothieno[2,3-c]pyran-7,4′-piperidine]-4-ol (825)

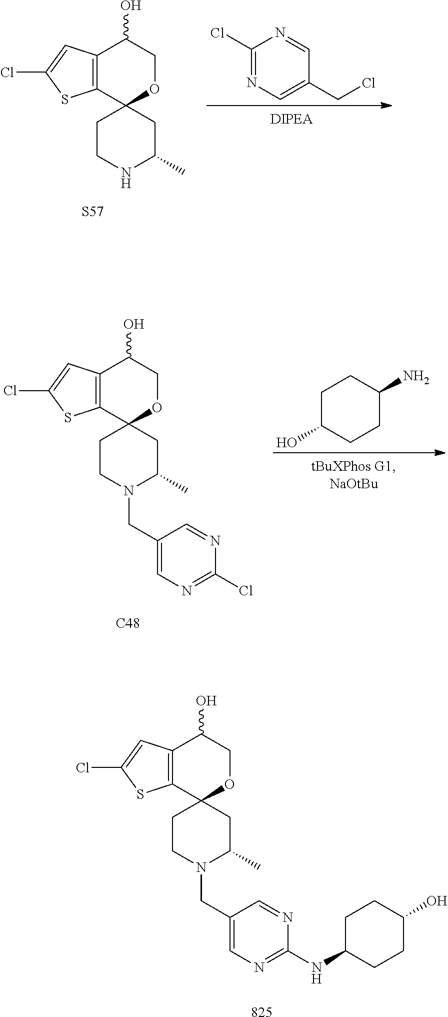

### Step 1. Synthesis of (2′S,7R)-2-chloro-1′-[(2-chloropyrimidin-5-yl)methyl]-2′-methyl-spiro[4,5-dihydrothieno[2,3-c]pyran-7,4′-piperidine]-4-ol (C48)

(2′S,7R)-2-chloro-2′-methyl-spiro[4,5-dihydrothieno[2,3-c]pyran-7,4′-piperidine]-4-ol S57 (73 mg, 0.2666 mmol) was dissolved in DCE (2 mL) and 2-chloro-5-(chloromethyl)pyrimidine (43 mg, 0.2638 mmol) and DIPEA (103 mg, 0.7969 mmol) were added. When the reaction was completed, the reaction mixture was diluted with DCM and water and separated in a phase separator. The crude product was then purified by silica gel chromatography (Gradient: 0-20% MeOH in DCM) to give (2′S,7R)-2-chloro-1′-[(2-chloropyrimidin-5-yl)methyl]-2′-methyl-spiro[4,5-dihydrothieno[2,3-c]pyran-7,4′-piperidine]-4-ol (76 mg, 64%). LCMS m/z 400.01 [M+H]⁺.

### Step 2. Synthesis of (2′S, 7R)-2-chloro-1′-[[2-[(4-hydroxycyclohexyl)amino]pyrimidin-5-yl]methyl]-2′-methyl-spiro[4,5-dihydrothieno[2,3-c]pyran-7,4′-piperidine]-4-ol (825)

To a flask with (2′S,7R)-2-chloro-1′-[(2-chloropyrimidin-5-yl)methyl]-2′-methyl-spiro[4,5-dihydrothieno[2,3-c]pyran-7,4′-piperidine]-4-ol C48 (76 mg, 0.1633 mmol), 4-aminocyclohexanol (22.57 mg, 0.1960 mmol), tBuXPhos G1 (10.6 mg, 0.01628 mmol) and tBuOH (2.27 mL). The reaction was degassed with N₂ before adding NaOtBu (34.5 mg, 0.3590 mmol). The flask was sealed and heated for 40 min at 60° C. and then quenched with MeOH. The solvent was removed and the product was redissolved in DCM and stirred with resin overnight to remove excess palladium. The resin was filtered off and the product was purified by silica gel chromatography (Gradient: 0-20% MeOH in DCM). Further purification was completed by reversed-phase HPLC. Method: C18 Waters Sunfire column (30×150 mm, 5 micron). Gradient: MeCN in H₂O with 0.1% trifluoroacetic acid. The product was isolated as (2′S,7R)-2-chloro-1′-[[2-[(4-hydroxycyclohexyl)amino]pyrimidin-5-yl]methyl]-2′-methyl-spiro[4,5-dihydrothieno[2,3-c]pyran-7,4′-piperidine]-4-ol (3.1 mg, 4%). LCMS m/z 481.19 [M+H]⁺.

### Compound 826

### 2-chloro-1′-[[4-(cyclopropylamino)-6-methyl-pyrazolo[1,5-a]pyrazin-2-yl]methyl]-2′-methyl-spiro[4,5-dihydrothieno[2,3-c]pyran-7,4′-piperidine]-4-ol (826)

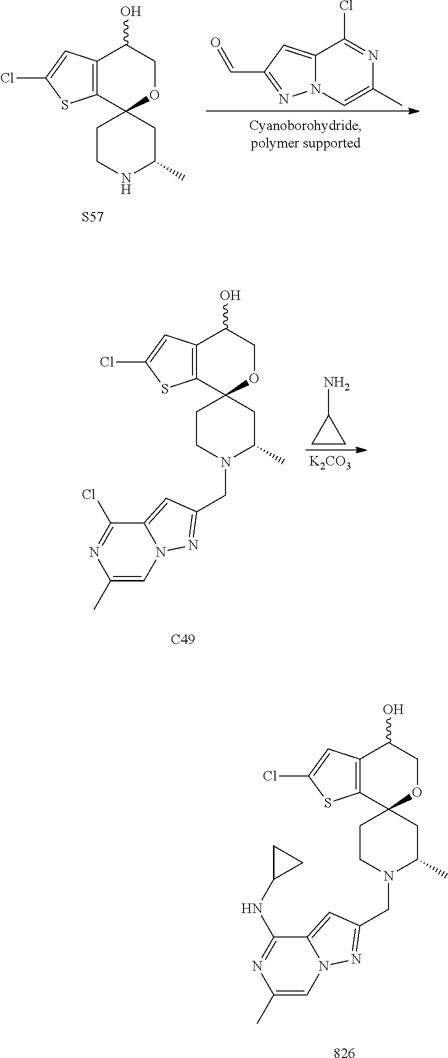

### Step 1. Synthesis of (2′S, 7R)-2-chloro-1′-[(4-chloro-6-methyl-pyrazolo[1,5-a]pyrazin-2-yl)methyl]-2′-methyl-spiro[4,5-dihydrothieno[2,3-c]pyran-7,4′-piperidine]-4-ol (C49)

(2′S,7R)-2-chloro-2′-methyl-spiro[4,5-dihydrothieno[2,3-c]pyran-7,4′-piperidine]-4-ol S57 (39.5 mg, 0.1356 mmol) and 4-chloro-6-methyl-pyrazolo[1,5-a]pyrazine-2-carbaldehyde (27.8 mg, 0.1421 mmol) were dissolved in DCM (1 mL) and acetic acid (60 μL, 1.055 mmol) was added to the solution followed by cyanoborohydride, polymer supported (280 mg of 2 mmol/g, 0.5600 mmol). The solution was capped and heated to 70° C. for 75 min. An additional portion of 4-chloro-6-methyl-pyrazolo[1,5-a]pyrazine-2-carbaldehyde (15 mg, 0.07668 mmol) was added and the solution was recapped stirred overnight. The suspension was stirred in MeOH (1.5 mL) for 10 min before filtering off the resin (twice). The solvent was removed and the residue was dissolved into water (2 mL) and DCM (2 mL). The pH of the aqueous layer was adjusted with 2M NaOH to pH >10. The phases were separated through a phase separator and the aqueous layer was extracted with DCM (2×10 mL) and the combined organics were concentrated under vacuum. The crude residue was purified by silica gel chromatography (Gradient: 0-20% MeOH in DCM) to yield (2′S,7R)-2-chloro-1′-[(4-chloro-6-methyl-pyrazolo[1,5-a]pyrazin-2-yl)methyl]-2′-methyl-spiro[4,5-dihydrothieno[2,3-c]pyran-7,4′-piperidine]-4-ol (31 mg, 48%). ¹H NMR (400 MHz, Chloroform-d) δ 8.10 (q, J=1.0 Hz, 1H), 6.80 (d, J=2.1 Hz, 1H), 6.75 (s, 1H), 4.45-4.34 (m, 1H), 4.19 (dd, J=14.2, 3.0 Hz, 1H), 3.99-3.71 (m, 3H), 2.84-2.56 (m, 2H), 2.50 (d, J=1.0 Hz, 3H), 2.12-1.91 (m, 4H), 1.81-1.67 (m, 1H), 1.54 (s, 1H), 1.26 (t, J=6.4 Hz, 3H). LCMS m/z 453.04 [M+H]⁺.

### Step 2. Synthesis of 2-chloro-1′-[[4-(cyclopropylamino)-6-methyl-pyrazolo[1,5-a]pyrazin-2-yl]methyl]-2′-methyl-spiro[4,5-dihydrothieno[2,3-c]pyran-7,4′-piperidine]-4-ol (826)

The compound 2-chloro-1′-[(4-chloro-6-methyl-pyrazolo[1,5-a]pyrazin-2-yl)methyl]-2′-methyl-spiro[4,5-dihydrothieno[2,3-c]pyran-7,4′-piperidine]-4-ol C49 (11.1 mg, 0.02448 mmol) was dissolved in DMF (0.8 mL) and cyclopropanamine (20 μL, 0.2886 mmol). To this was added K₂CO₃ (13.56 mg, 0.09811 mmol) and the reaction was heated to 45° C. for 90 min, then heated to 80° C. and stirred overnight. The reaction was then diluted in water and DCM, separated the layers, extracted the aqueous with DCM (2×), and passed the pooled organics through a phase separator. The reaction was concentrated and purified by reversed-phase HPLC. Method: C18 Waters Sunfire column (30×150 mm, 5 micron). Gradient: MeCN in H₂O with 10 mM ammonium hydroxide. The product was isolated as 2-chloro-1′-[[4-(cyclopropylamino)-6-methyl-pyrazolo[1,5-a]pyrazin-2-yl]methyl]-2′-methyl-spiro[4,5-dihydrothieno[2,3-c]pyran-7,4′-piperidine]-4-ol (2.8 mg, 23%). ¹H NMR (400 MHz, Methanol-d₄) δ 7.65 (q, J=1.1 Hz, 1H), 6.86 (d, J=0.9 Hz, 2H), 4.40 (dt, J=8.9, 3.9 Hz, 1H), 4.22 (d, J=14.2 Hz, 1H), 4.03 (d, J=14.3 Hz, 1H), 3.91 (ddd, J=12.2, 9.6, 3.6 Hz, 1H), 3.75-3.63 (m, 1H), 2.91 (tt, J=7.1, 3.7 Hz, 2H), 2.85-2.75 (m, 1H), 2.33 (d, J=1.0 Hz, 3H), 2.21-2.07 (m, 2H), 1.99-1.54 (m, 3H), 1.36 (t, J=5.7 Hz, 3H), 0.86 (td, J=7.0, 4.9 Hz, 2H), 0.68-0.59 (m, 2H). LCMS m/z 474.12 [M+H]⁺.

### Preparation S59

### (2′S,4S,7R)-2-chloro-4-(difluoromethyl)-2′-methyl-spiro[5H-thieno[2,3-c]pyran-7,4′-piperidine]-4-ol (S59)

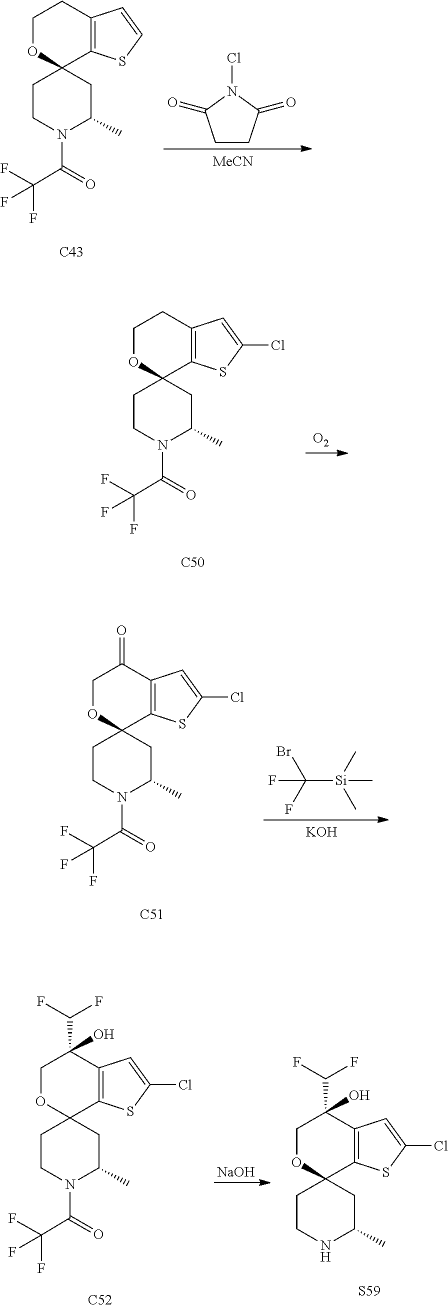

### Step 1. 1-[(2′S, 7R)-2-chloro-2′-methyl-spiro[4,5-dihydrothieno[2,3-c]pyran-7,4′-piperidine]-1′-yl]-2,2,2-trifluoro-ethanoneone (C50)

To a solution of 2,2,2-trifluoro-1-[(2′S,7R)-2′-methylspiro[4,5-dihydrothieno[2,3-c]pyran-7,4′-piperidine]-1′-yl]ethanone C43 (440 g, 1.378 mol)) in MeCN (3.6 L), stirred for 15 minutes at room temperature under a nitrogen atmosphere was added DMAP (9.2 g, 75.31 mmol), followed by 1-chloropyrrolidine-2,5-dione (262 g, 1.962 mol). The resulting reaction mixture was warmed to 70° C., and stirred for 14 hours. The reaction mixture was cooled to room temperature and quenched with 10% aqueous sodium bisulfite (˜1 L) and stirred for 20 minutes. The mixture was partitioned between water (˜1 L) and ethyl acetate (˜2 L). The aqueous layer was extracted with ethyl acetate (˜1 L). The combined organic phase was washed successively with 1:1 water:brine, 1:1:1 water:brine: 1 M aq HCl, and brine (˜1 L each), dried with MgSO₄, filtered, and concentrated. Silica gel purification (Column: Combiflash Torrent, 2×1600 g. Gradient; 0-40% EtOAc in heptane), afforded 1-[(2′S,7R)-2-chloro-2′-methyl-spiro[4,5-dihydrothieno[2,3-c]pyran-7,4′-piperidine]-1′-yl]-2,2,2-trifluoro-ethanone C50 (418 g, 75%) ¹H NMR (300 MHz, Chloroform-d) δ 6.57 (s, 1H), 4.36 (s, 1H), 3.96-3.68 (m, 3H), 3.39 (s, 1H), 2.56 (t, J=5.5 Hz, 2H), 2.30 (ddd, J=14.2, 6.3, 1.8 Hz, 2H), 1.99-1.79 (m, 1H), 1.73 (dd, J=14.4, 10.6 Hz, 1H), 1.29 (d, J=6.5 Hz, 3H). 19F NMR (282 MHz, Chloroform-d) δ−69.80. LCMS m/z 354.12 [M+1]⁺.

### Step 2. (2S,4R)-2′-chloro-2-methyl-1-(2,2,2-trifluoroacetyl)spiro[piperidine-4,7′-thieno[2,3-c]pyran]-4′-one (C51)

To a mixture of 1-[(2′S,7R)-2-chloro-2′-methyl-spiro[4,5-dihydrothieno[2,3-c]pyran-7,4′-piperidine]-1′-yl]-2,2,2-trifluoro-ethanone C50 (6 g, 16.96 mmol) in acetonitrile (60 mL) was added N-hydroxyphthalimide (1.7 g, 10.42 mmol) and cobaltous; diacetate; tetrahydrate (270 mg, 1.084 mmol), and then the mixture was vacuum purged with an oxygen balloon three times. The reaction mixture was heated to 45° C. and stirred for 6 hours. The reaction was cooled to room temperature. The mixture was purged with nitrogen three times and then diluted with MTBE (100 mL) and sat. aq. bicarbonate (100 mL). The layers were separated and the organic layer was washed with water (2×50 mL) and brine (50 mL). The organic layer was dried with sodium sulfate, filtered and concentrated. Purification by silica gel chromatography (0-30% EtOAc:heptane) yielded the product (2S,4R)-2′-chloro-2-methyl-1-(2,2,2-trifluoroacetyl)spiro[piperidine-4,7′-thieno[2,3-c]pyran]-4′-one (2700 mg, 42%) ¹H NMR (400 MHz, Chloroform-d) δ 7.19 (s, 1H), 4.38 (d, J=6.1 Hz, 1H), 4.35-4.20 (m, 2H), 3.95 (s, 1H), 3.46 (s, 1H), 2.60-2.43 (m, 2H), 2.00-1.90 (m, 1H), 1.85 (dd, J=14.5, 10.6 Hz, 1H), 1.34 (d, J=6.5 Hz, 3H). LCMS m/z 367.96 [M+1]⁺.

### Step 3. 1-[(2′S,4S,7R)-2-chloro-4-(difluoromethyl)-4-hydroxy-2′-methyl-spiro[5H-thieno[2,3-c]pyran-7,4′-piperidine]-1′-yl]-2,2,2-trifluoro-ethanone (C52)

To a solution of (2S,4R)-2′-chloro-2-methyl-1-(2,2,2-trifluoroacetyl)spiro[piperidine-4,7′-thieno[2,3-c]pyran]-4′-one C51 (11.3 g, 30.73 mmol) in MeCN (115 mL) at room temperature was added DMPU (7.5 mL, 62.26 mmol) followed by PPh₃ (9.7 g, 36.98 mmol) followed by [bromo(difluoro)methyl]-trimethyl-silane (10.8 g, 53.18 mmol). Resulting solution was heated at 55° C. for 1 hour. Reaction was cooled to room temperature and KOH (140 mL of 1 M, 140.0 mmol) was added. Partial deprotection was observed. The reaction was stirred for 30 min before adding saturated aqueous NaHCO₃ (300 mL) until achieving a pH of 8-9. Reaction was extracted with EtOAc (500 mL, 200 mL). The combined organic extracts were dried with MgSO₄, filtered and concentrated. Crude was dissolved in DCM (200 mL), and treated with Et₃N (13 mL, 93.27 mmol) followed by TFAA (4.5 mL, 32.37 mmol). The reaction was stirred for 45 minutes. The reaction was quenched with saturated aqueous NaHCO₃ (200 mL). Layers were separated, aqueous was re-extracted with DCM (100 mL) and combined organics were dried with MgSO₄, filtered and concentrated. Purification by silica gel chromatography (0-30% EtOAc in Heptane) yielded the product and a minor amount of the other diastereomer. Further purification by silica gel chromatography (Gradient: 0-60% EtOAc:heptane) yielded the product 1-[(2′S,4S,7R)-2-chloro-4-(difluoromethyl)-4-hydroxy-2′-methyl-spiro[5H-thieno[2,3-c]pyran-7,4′-piperidine]-1′-yl]-2,2,2-trifluoro-ethanone (5.4 g, 42%) as an off-white solid. ¹H NMR (400 MHz, Chloroform-d) δ 6.91 (d, J=1.3 Hz, 1H), 5.87 (dd, J=55.6, 54.8 Hz, 1H), 4.34 (s, 1H), 3.99 (d, J=12.2 Hz, 1H), 3.88 (s, 1H), 3.69 (ddd, J=12.2, 3.8, 1.2 Hz, 1H), 3.42 (s, 1H), 2.60 (d, J=1.1 Hz, 1H), 2.49-2.38 (m, 1H), 2.30 (q, J=11.7, 8.4 Hz, 1H), 2.02-1.91 (m, 1H), 1.73 (dd, J=14.7, 10.8 Hz, 1H), 1.30 (d, J=6.5 Hz, 3H). 19F NMR (376 MHz, Chloroform-d) δ−69.80, −130.52 (d, J=280.4 Hz), −135.69 (d, J=280.1 Hz).

### Step 4. (2′S,4S,7R)-2-chloro-4-(difluoromethyl)-2′-methyl-spiro[5H-thieno[2,3-c]pyran-7,4′-piperidine]-4-ol (S59)

To a solution of 1-[(2′S,4S,7R)-2-chloro-4-(difluoromethyl)-4-hydroxy-2′-methyl-spiro[5H-thieno[2,3-c]pyran-7,4′-piperidine]-1′-yl]-2,2,2-trifluoro-ethanone C52 (3.23 g, 7.694 mmol) in MeOH (50 mL) was added aq NaOH (12 mL of 2 M, 24.00 mmol). Reaction was stirred for 2 hours. The solvent was removed in vacuo and the residue was partitioned between 2-MeTHF and saturated aqueous sodium bicarbonate (200 mL each). Organic layer was separated and washed with saturated aqueous sodium bicarbonate followed by brine (150 mL each). The combined organic extracts were dried with MgSO₄, filtered and concentrated. Residue was dissolved in EtOAc (100 mL refluxing) to give a slightly cloudy solution. Heptane (35 mL) was added, and the resulting solution/suspension was heated to reflux for 30 sec. The solution was allowed to stand at room temperature. After 18 h, crystals were isolated via filtration, washed with heptane (20 mL), and dried to give (2′S,4S,7R)-2-chloro-4-(difluoromethyl)-2′-methyl-spiro[5H-thieno[2,3-c]pyran-7,4′-piperidine]-4-ol (1.9 g, 76%) as pale cream-colored crystals. ¹H NMR (300 MHz, DMSO-d6) δ 7.05 (s, 1H), 6.22 (s, 1H), 6.09 (t, J=55.1 Hz, 1H), 3.93 (d, J=12.2 Hz, 1H), 3.66 (dd, J=12.1, 2.8 Hz, 1H), 3.34 (s, 1H), 3.03-2.80 (m, 3H), 2.19-2.07 (m, 1H), 1.87 (dd, J=13.8, 2.5 Hz, 1H), 1.67 (dt, J=13.5, 8.2 Hz, 1H), 1.27 (dd, J=13.8, 11.4 Hz, 1H), 1.02 (d, J=6.3 Hz, 3H). 19F NMR (282 MHz, DMSO-d6) δ−128.66 (d, J=277.3 Hz), −134.33 (d, J=277.4 Hz). LCMS m/z 324.06 [M+1]⁺.

### Preparation S60

### (2′S, 7R)-2-chloro-4-(difluoromethyl)-2′-methyl-spiro[5H-thieno[2,3-c]pyran-7,4′-piperidine]-4-ol (S60)

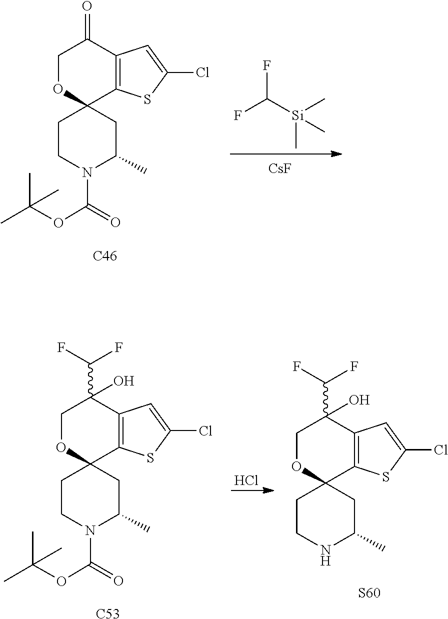

### Step 1. tert-butyl (2′S, 7R)-2-chloro-4-(difluoromethyl)-4-hydroxy-2′-methyl-spiro[5H-thieno[2,3-c]pyran-7,4′-piperidine]-1′-carboxylate (C53)

To a solution of tert-butyl (2S,4R)-2′-chloro-2-methyl-4′-oxo-spiro[piperidine-4,7′-thieno[2,3-c]pyran]-1-carboxylate C46 (1.05 g, 2.823 mmol) in 1,2-dimethoxyethane (30 mL) was added, cesium fluoride (430 mg, 2.831 mmol) and 1,4,7,10,13,16-hexaoxacyclooctadecane (750 mg, 2.838 mmol) under nitrogen. To the mixture was added difluoromethyl(trimethyl)silane (2.3 mL, 16.85 mmol) and the resulting mixture was stirred at room temperature overnight. TBAF (2.5 mL of 1 M, 2.500 mmol) was added to the reaction and the solution was stirred at room temperature for an additional 30 minutes. Reaction was quenched by partitioning between water and dichloromethane. The organics were collected through a phase separator and concentrated in vacuo. Purification by silica gel chromatography (Gradient: 0-30% EtOAc in Heptanes) afforded tert-butyl (2′S,7R)-2-chloro-4-(difluoromethyl)-4-hydroxy-2′-methyl-spiro[5H-thieno[2,3-c]pyran-7,4′-piperidine]-1′-carboxylate (440 mg, 34%) ¹H NMR (300 MHz, Chloroform-d) δ 6.89 (d, J=1.4 Hz, 1H), 5.88 (t, J=55.3 Hz, 1H), 4.04-3.91 (m, 2H), 3.80-3.67 (m, 2H), 3.40-3.23 (m, 1H), 2.46 (d, J=1.0 Hz, 1H), 2.33-2.01 (m, 2H), 1.85-1.60 (m, 2H), 1.47 (s, 9H), 1.26 (dd, J=6.6, 5.2 Hz, 3H). LCMS m/z 424.29 [M+1]⁺.

### Step 2. (2′S, 7R)-2-chloro-4-(difluoromethyl)-2′-methyl-spiro[5H-thieno[2,3-c]pyran-7,4′-piperidine]-4-ol (S60)

A solution of tert-butyl (2′S,7R)-2-chloro-4-(difluoromethyl)-4-hydroxy-2′-methyl-spiro[5H-thieno[2,3-c]pyran-7,4′-piperidine]-1′-carboxylate C53 (440 mg, 0.9582 mmol) in 1,4-dioxane (5 mL) was treated with HCl (5 mL of 4 M, 20.00 mmol) and stirred at room temperature overnight. The solution was concentrated in vacuo to afford (2′S,7R)-2-chloro-4-(difluoromethyl)-2′-methyl-spiro[5H-thieno[2,3-c]pyran-7,4′-piperidine]-4-ol (Hydrochloride salt) (340 mg, 86%) LCMS m/z 323.97 [M+1]⁺.

### Preparation S61

### (2′S,4S,7R)-2-chloro-4-(difluoromethyl)-4-methoxy-2′-methyl-spiro[5H-thieno[2,3-c]pyran-7,4′-piperidine](S61)

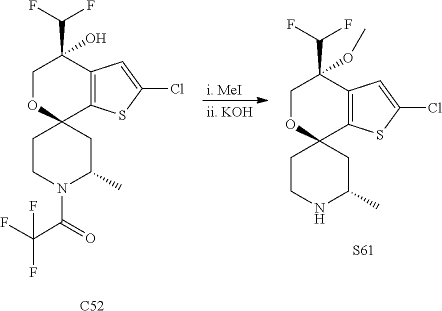

### Preparation of (2′S,4S,7R)-2-chloro-4-(difluoromethyl)-4-methoxy-2′-methyl-spiro[5H-thieno[2,3-c]pyran-7,4′-piperidine](S61)

To a solution of 1-[(2′S,4S,7R)-2-chloro-4-(difluoromethyl)-4-hydroxy-2′-methyl-spiro[5H-thieno[2,3-c]pyran-7,4′-piperidine]-1′-yl]-2,2,2-trifluoro-ethanone C52 (50 mg, 0.1191 mmol) and dicesium carbonate (40 mg, 0.1228 mmol) in DMF (1 mL) under nitrogen was added MeI (10 μL, 0.1606 mmol). Reaction was stirred overnight. Reaction was quenched with water and extracted with DCM. Solvent was removed in vacuo. Aqueous potassium hydroxide (500 μL of 2 M, 1.000 mmol) (100 μL, 5.551 mmol) was added and reaction was stirred overnight. Reaction was diluted with DCM and extracted (3×) through a phase separator. Solvent was removed in vacuo. Purification by reversed-phase HPLC. Method: C18 Waters Sunfire column (30×150 mm, 5 micron). Gradient: MeCN in H₂O with 5 mM HCl. (2′S,4S,7R)-2-chloro-4-(difluoromethyl)-4-methoxy-2′-methyl-spiro[5H-thieno[2,3-c]pyran-7,4′-piperidine] (Hydrochloride salt) (14 mg, 31%) LCMS m/z 338.21 [M+1]⁺. ¹H NMR (300 MHz, DMSO-d6) δ 8.95 (s, 1H), 8.69 (s, 1H), 7.17 (s, 1H), 6.32 (t, J=54.4 Hz, 1H), 4.09 (s, 2H), 3.33 (s, 2H), 3.18 (s, 3H), 3.04 (s, 1H), 2.22 (m, 2H), 1.92 (m, 2H), 1.25 (d, J=6.4 Hz, 3H).

### Compound 827

### (2′S,4S,7R)-2-chloro-4-(difluoromethyl)-2′-methyl-1′-[[1-(2-methylsulfonylethyl)pyrazol-4-yl]methyl]spiro[5H-thieno[2,3-c]pyran-7,4′-piperidine]-4-ol (827)

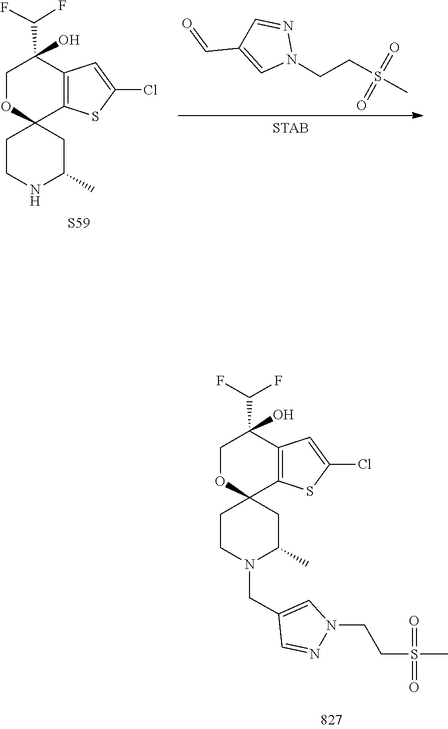 [SMILES: C[C@H]1C[C@@]2(CCN1)OC[C@](O)(C(F)F)c1cc(Cl)sc12 ; CS(=O)(=O)CCn1cc(C=O)cn1 ; "STAB" ; "S59" ; C[C@H]1C[C@@]2(CCN1Cc1cnn(CCS(C)(=O)=O)c1)OC[C@](O)(C(F)F)c1cc(Cl)sc12]

### (2′S,4S,7R)-2-chloro-4-(difluoromethyl)-2′-methyl-1′-[[1-(2-methylsulfonylethyl)pyrazol-4-yl]methyl]spiro[5H-thieno[2,3-c]pyran-7,4′-piperidine]-4-ol (827)

To a solution of 1-(2-methylsulfonylethyl)pyrazole-4-carbaldehyde (1.23 g, 6.082 mmol) and (2′S,4S,7R)-2-chloro-4-(difluoromethyl)-2′-methyl-spiro[5H-thieno[2,3-c]pyran-7,4′-piperidine]-4-ol S59 (1.79 g, 5.528 mmol) in THF (26 mL) at room temperature was added triacetoxyboranuide (Sodium salt) (2.50 g, 11.80 mmol). Reaction was heated to 50° C. and stirred for 3 hours. Reaction was cooled in an ice-water bath and carefully quenched with saturated aqueous sodium bicarbonate (100 mL). Resulting mixture was stirred at room temperature for 1 hour, and then EtOAc and water (50 mL each) were added. Reaction was stirred for a further 1 hour. Bisphasic mixture was extracted with EtOAc (100 mL). Organic layer was washed with brine (100 mL) and combined organics were dried with MgSO₄, filtered and concentrated. Purification by silica gel chromatography (0-15% [2 M NH₃ in MeOH]/DCM) was followed by a second purification by silica gel chromatography (0-15% [2 M NH₃ in MeOH]/DCM). Product (1.65 g, 3.24 mmol) was suspended in water (20 mL) and treated with 1 M aq HCl (3.5 mL). Mixture was spun on rotovap (no vacuum), bath at 60° C. for 10 min to give a clear solution. Solution was filtered through a syringe filter (polyether sulfone membrane), Combined solution and wash was frozen and lyophilized to give (2′S,4S,7R)-2-chloro-4-(difluoromethyl)-2′-methyl-1′-[[1-(2-methylsulfonylethyl)pyrazol-4-yl]methyl]spiro[5H-thieno[2,3-c]pyran-7,4′-piperidine]-4-ol 827 (Hydrochloride salt) (1.77 g, 58%) as a white solid. ¹H NMR (400 MHz, Chloroform-d) δ 7.46 (d, J=0.7 Hz, 1H), 7.44 (d, J=0.7 Hz, 1H), 6.88 (d, J=1.1 Hz, 1H), 5.84 (t, J=55.3 Hz, 1H), 4.58 (t, J=6.0 Hz, 2H), 3.95 (d, J=12.1 Hz, 1H), 3.79 (d, J=14.4 Hz, 1H), 3.74-3.67 (m, 1H), 3.67-3.60 (m, 2H), 3.57 (d, J=14.4 Hz, 1H), 3.26 (s, 1H), 2.65 (ddd, J=11.4, 4.4, 2.5 Hz, 1H), 2.54-2.36 (m, 2H), 2.45 (s, 3H), 2.10-2.00 (m, 1H), 1.98-1.76 (m, 2H), 1.56 (dd, J=14.1, 11.4 Hz, 1H), 1.18 (d, J=6.2 Hz, 3H). 19F NMR (376 MHz, Chloroform-d) δ−129.99 (d, J=279.9 Hz), −135.47 (d, J=279.9 Hz). LCMS m/z 510.02 [M+1]⁺.

### Compound 828

### (2′S,4S,7R)-2-chloro-4-(difluoromethyl)-2′-methyl-1′-(1H-pyrazol-4-ylmethyl)spiro[5H-thieno[2,3-c]pyran-7,4′-piperidine]-4-ol (828)

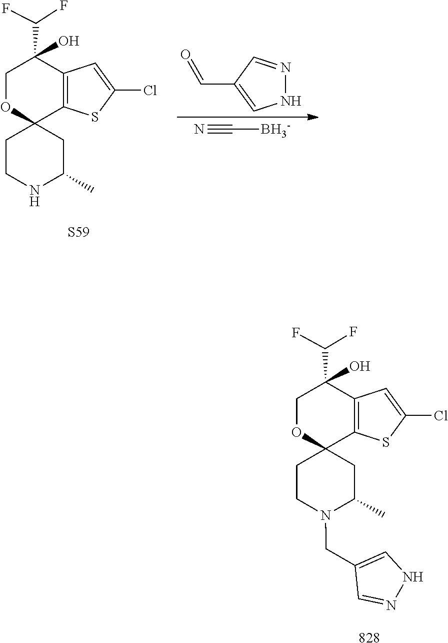

### (2′S,4S,7R)-2-chloro-4-(difluoromethyl)-2′-methyl-1′-(1H-pyrazol-4-ylmethyl)spiro[5H-thieno[2,3-c]pyran-7,4′-piperidine]-4-ol (828)

To a solution of (2′S,4S,7R)-2-chloro-4-(difluoromethyl)-2′-methyl-spiro[5H-thieno[2,3-c]pyran-7,4′-piperidine]-4-ol S59 (150 mg, 0.4633 mmol) in dichloromethane (4 mL) was added 1H-pyrazole-4-carbaldehyde (45 mg, 0.4683 mmol), acetic acid (134 mg, 2.231 mmol) and polymer supported cyanoborohydride (231 mg of 2 mmol/g, 0.4620 mmol) in a microwave vial. The solution was capped and heated to 95° C. in a microwave reactor for 120 minutes. MeOH was added and the solution was stirred for ten minutes to remove product from the polymer support beads. The solution was filtered and the solvent was removed in vacuo. Purification by reversed-phase HPLC. Method: C18 Waters Sunfire column (30×150 mm, 5 micron) Gradient: MeCN in H₂O 10 mM Ammonium Hydroxide afforded (2′S,4S,7R)-2-chloro-4-(difluoromethyl)-2′-methyl-1′-(1H-pyrazol-4-ylmethyl)spiro[5H-thieno[2,3-c]pyran-7,4′-piperidine]-4-ol (131 mg, 54%) ¹H NMR (300 MHz, Methanol-d4) δ 7.63 (s, 2H), 6.96 (s, 1H), 5.89 (t, J=55.2 Hz, 1H), 4.00 (dd, J=15.0, 13.2 Hz, 2H), 3.72 (dd, J=19.6, 13.1 Hz, 2H), 2.99-2.54 (m, 3H), 2.24 (d, J=14.3 Hz, 1H), 2.13-1.80 (m, 3H), 1.62 (dd, J=14.4, 11.6 Hz, 1H), 1.30 (d, J=6.3 Hz, 3H). LCMS m/z 404.03 [M+1]⁺.

### Compound 829

### 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]pyrazol-1-yl]-3-methyl-butane-2,3-diol (829)

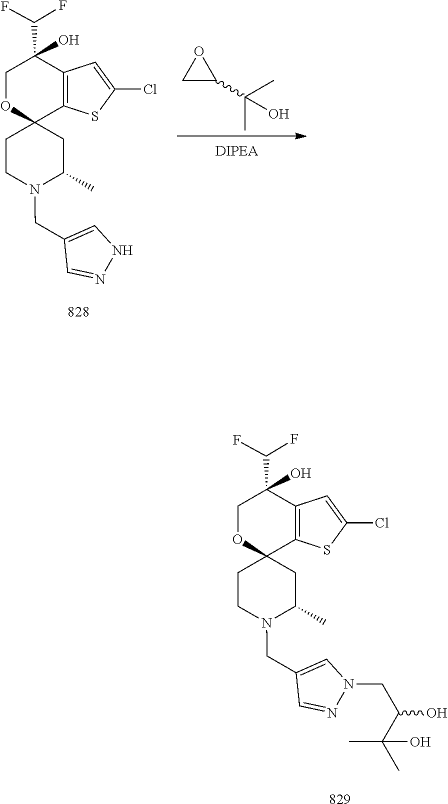

### 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]pyrazol-1-yl]-3-methyl-butane-2,3-diol (829)

To a solution of (2′S,4R,7R)-2-chloro-4-(difluoromethyl)-2′-methyl-1′-(1H-pyrazol-4-ylmethyl)spiro[5H-thieno[2,3-c]pyran-7,4′-piperidine]-4-ol 828 (85 mg, 0.2105 mmol) and 2-(oxiran-2-yl)propan-2-ol (34 mg, 0.3329 mmol) in MeOH (1.45 mL) was added DIPEA (183 μL, 1.051 mmol) in a microwave vial. The reaction was stirred at 90° C. for 1 hour. Reaction was transferred to the microwave and run for seven hours at 120° C. Solvent was removed in vacuo. Purification by reversed-phase HPLC. Method: C18 Waters Sunfire column (30×150 mm, 5 micron). Gradient: MeCN in H₂O with 0.1% trifluoroacetic acid yielded 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]pyrazol-1-yl]-3-methyl-butane-2,3-diol (Trifluoroacetate salt) (16 mg, 12%) ¹H NMR (300 MHz, Methanol-d4) δ 7.68 (s, 1H), 7.50 (s, 1H), 6.96 (s, 1H), 5.89 (t, J=55.3 Hz, 1H), 4.44 (dd, J=13.9, 2.3 Hz, 1H), 4.18-3.90 (m, 3H), 3.71 (td, J=10.9, 9.9, 3.0 Hz, 3H), 2.97-2.58 (m, 3H), 2.24 (dt, J=14.3, 2.9 Hz, 1H), 1.94 (dtd, J=26.7, 13.9, 3.6 Hz, 2H), 1.62 (dd, J=14.3, 11.6 Hz, 1H), 1.29 (d, J=6.3 Hz, 3H), 1.23 (d, J=8.3 Hz, 6H). LCMS m/z 506.14 [M+1]⁺.

### Compounds 830-835

Compounds 830-835 were prepared from reductive amination step as describes for compounds 829 with the relevant aldehydes and piperidines. Aldehydes and piperidines were prepared by methods described above or obtained from commercial sources. Any modifications to methods are noted in Table 35 and accompanying footnotes.

TABLE 35
Method of preparation, structure and physicochemical data for compounds 830-835.

| | | Aldehyde and | | |
|---|---|---|---|---|
| Compd | Product | piperidine reagents | Method | ¹H NMR |
| 830 | 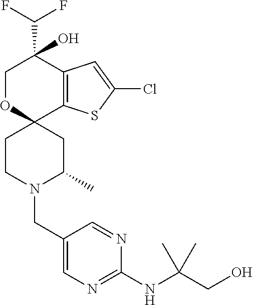 [SMILES: C[C@H]1C[C@@]2(CCN1Cc1cnc(NC(C)(C)CO)nc1)OC[C@](O)(C(F)F)c1cc(Cl)sc12] | S59; 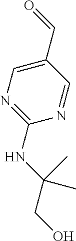 | 829 | ¹H NMR (300 MHz, Methanol-d4) δ 8.23 (s, 2H), 6.95 (s, 1H), 5.90 (t, J = 55.2 Hz, 1H), 3.98 (t, J = 12.7 Hz, 2H), 3.84- 3.59 (m, 3H), 3.22-2.98 (m, 1H), 2.72-2.53 (m, 2H), 2.41 (td, J = 12.2, 2.8 Hz, 1H), 2.20 (dt, J = 14.3, 2.8 Hz, 1H), 2.03- 1.67 (m, 2H), 1.56 (dd, J = 14.2, 11.4 Hz, 1H), 1.37 (s, 6H), 1.24 (d, J = 6.2 Hz, 3H). LCMS m/z 503.05 [M + 1]⁺. |
| 831 | 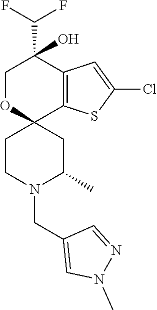 [SMILES: C[C@H]1C[C@@]2(CCN1Cc1cnn(C)c1)OC[C@](O)(C(F)F)c1cc(Cl)sc12] | S59; 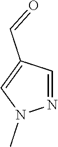 | 829^{1,2} | ¹H NMR (300 MHz, Methanol-d4) δ 7.87 (s, 1H), 7.66 (s, 1H), 7.03 (s, 1H), 5.94 (t, J = 55.2 Hz, 1H), 4.55 (d, J = 14.0 Hz, 1H), 4.27 (d, J = 14.1 Hz, 1H), 4.05 (d, J = 12.2 Hz, 1H), 3.94 (s, 4H), 3.74 (d, J = 12.3 Hz, 1H), 3.47 (s, 2H), 2.54 (d, J = 14.7 Hz, 1H), 2.26 (d, J = 15.5 Hz, 1H), 2.13-1.71 (m, 2H), 1.54 (d, J = 6.5 Hz, 3H). LCMS m/z 418.14 [M + 1]⁺. |
| 832 | 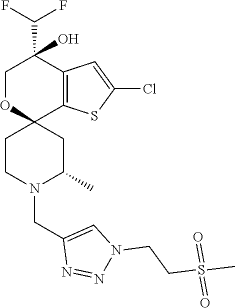 [SMILES: C[C@H]1C[C@@]2(CCN1Cc1cn(CCS(C)(=O)=O)nn1)OC[C@](O)(C(F)F)c1cc(Cl)sc12] | S59; 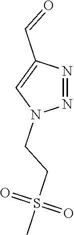 | 829^{1,2} | LCMS m/z 511.11 [M + 1]⁺. |
| 833 | 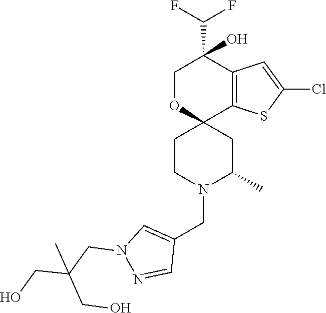 [SMILES: C[C@H]1C[C@@]2(CCN1Cc1cnn(CC(C)(CO)CO)c1)OC[C@](O)(C(F)F)c1cc(Cl)sc12] | S59; 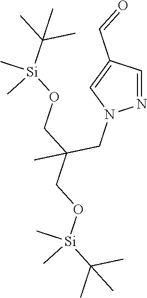 | 829³ | ¹H NMR (400 MHz, Methanol-d4) δ 7.62 (d, J = 0.7 Hz, 1H), 7.45 (d, J = 0.8 Hz, 1H), 6.94 (d, J = 0.9 Hz, 1H), 5.89 (t, J = 55.2 Hz, 1H), 4.13 (s, 2H), 3.89 (dd, J = 41.8, 13.2 Hz, 2H), 3.66 (dd, J = 13.2, 6.8 Hz, 2H), 3.43- 3.32 (m, 4H), 2.80-2.43 (m, 3H), 2.18 (dt, J = 14.2, 2.9 Hz, 1H), 2.03-1.69 (m, 2H), 1.54 (dd, J = 14.2, 11.5 Hz, 1H), 1.24 (d, J = 6.3 Hz, 3H), 0.82 (s, 3H). LCMS m/z 506.14 [M + 1]⁺. |
| 834 | 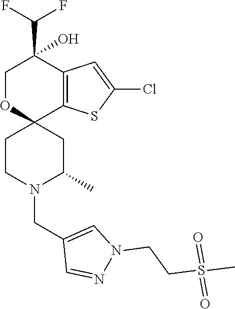 [SMILES: C[C@H]1C[C@@]2(CCN1Cc1cnn(CCS(C)(=O)=O)c1)OC[C@@](O)(C(F)F)c1cc(Cl)sc12] | S60; 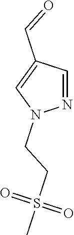 | 829^{1,4,5} | ¹H NMR (300 MHz, Methanol-d4) δ 7.75 (s, 1H), 7.55 (d, J = 0.7 Hz, 1H), 6.95 (d, J = 1.0 Hz, 1H), 5.90 (t, J = 55.2 Hz, 1H), 4.64 (dd, J = 6.9, 5.9 Hz, 2H), 3.94 (dd, J = 13.2, 8.2 Hz, 2H), 3.79- 3.60 (m, 4H), 2.85-2.57 (m, 6H), 2.22 (dd, J = 14.4, 3.1 Hz, 1H), 2.06- 1.96 (m, 1H), 1.84-1.63 (m, 2H), 1.26 (d, J = 6.3 Hz, 3H). LCMS m/z 510.11 [M + 1]⁺. |
| 835 | 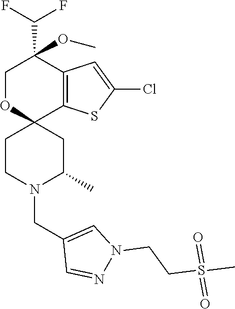 [SMILES: CO[C@]1(C(F)F)CO[C@@]2(CCN(Cc3cnn(CCS(C)(=O)=O)c3)[C@@H](C)C2)c2sc(Cl)cc21] | S61; 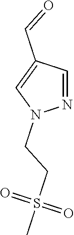 | 829^{1,6,7} | ¹H NMR (300 MHz, Chloroform-d) δ 7.51 (s, 1H), 7.46 (s, 1H), 6.83 (s, 1H), 5.79 (t, J = 55.1 Hz, 1H), 4.61 (t, J = 6.0 Hz, 2H), 4.09-3.95 (m, 2H), 3.84 (d, J = 14.4 Hz, 1H), 3.66 (t, J = 6.1 Hz, 2H), 3.57 (d, J = 14.4 Hz, 1H), 3.27 (s, 3H), 2.72-2.64 (m, 1H), 2.64-2.51 (m, 1H), 2.48 (d, J = 1.0 Hz, 3H), 2.47-2.33 (m, 1H), 2.13-1.91 (m, 2H), 1.83- 1.60 (m, 2H), 1.23 (d, J = 6.2 Hz, 3H). LCMS m/z 523.99 [M + 1]⁺. |

Footnotes:
¹Reaction was heated in the microwave at 110° C. for 45 minutes.
²Purification by reversed-phase HPLC. Method: C18 Waters Sunfire column (30 × 150 mm, 5 micron). Gradient: MeCN in H₂O with 0.1% trifluoroacetic acid yielded the product.
³After the beads were washed with MeOH and filtered 4M HCl in dioxane (10 eq) was added. Reaction was stirred for ten minutes to remove TBS protecting groups.
⁴After completion of the reaction the solution was filtered and the solvent was removed in vacuo. Purification by silica gel chromatography (Gradient: 0-100% EtOAc in Heptanes) yielded the product.
⁵SFC purification gave the compound with R stereo-chemistry at the benzylic position.
⁶After completion of the reaction the solution was filtered and the solvent was removed in vacuo. Purification by silica gel chromatography (Gradient: 0-20% MeOH in DCM) yielded the product.
⁷Acetic Acid was omitted was the reaction since the piperidine starting material was an HCl salt.

### Preparation S62

### (2′S,4R,7R)-2-chloro-4-(difluoromethyl)-2′-methyl-1′-prop-2-ynyl-spiro[5H-thieno[2,3-c]pyran-7,4′-piperidine]-4-ol (S62)

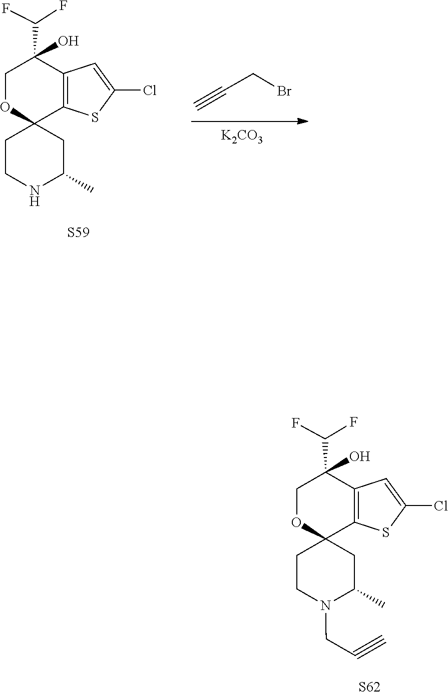

### Preparation of (2′S,4R,7R)-2-chloro-4-(difluoromethyl)-2′-methyl-1′-prop-2-ynyl-spiro[5H-thieno[2,3-c]pyran-7,4′-piperidine]-4-ol (S62)

A solution of (2′S,4R,7R)-2-chloro-4-(difluoromethyl)-2′-methyl-spiro[5H-thieno[2,3-c]pyran-7,4′-piperidine]-4-ol S59 (320 mg, 0.9883 mmol) and potassium carbonate (178 mg, 1.288 mmol) in tetrahydrofuran (6.4 mL) was heated to 50° C. and stirred. At this time propargyl bromide (191 mg, 1.284 mmol) in toluene was added and the mixture was stirred. After stirring overnight, the reaction was quenched with aqueous sodium bicarbonate and DCM and the organic layer was collected through a phase separator. The solvent was removed in vacuo. Purification by silica gel chromatography (Gradient: 0-20% MeOH:DCM). yielded (2′S,4R,7R)-2-chloro-4-(difluoromethyl)-2′-methyl-1′-prop-2-ynyl-spiro[5H-thieno[2,3-c]pyran-7,4′-piperidine]-4-ol (223 mg, 61%) ¹H NMR (300 MHz, Chloroform-d) δ 6.91 (d, J=1.3 Hz, 1H), 5.91 (t, J=55.3 Hz, 1H), 4.07 (d, J=12.2 Hz, 1H), 3.90-3.75 (m, 1H), 3.67 (dd, J=17.4, 2.4 Hz, 1H), 3.42 (dd, J=17.5, 2.4 Hz, 1H), 3.02-2.62 (m, 3H), 2.48 (s, 1H), 2.26 (t, J=2.4 Hz, 1H), 2.13 (dt, J=14.1, 2.8 Hz, 1H), 2.03-1.85 (m, 2H), 1.11 (d, J=6.3 Hz, 3H). LCMS m/z 362.0 [M+1]⁺.

### Compound 836

### 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]triazol-1-yl]-3-methyl-butane-2,3-diol (836)

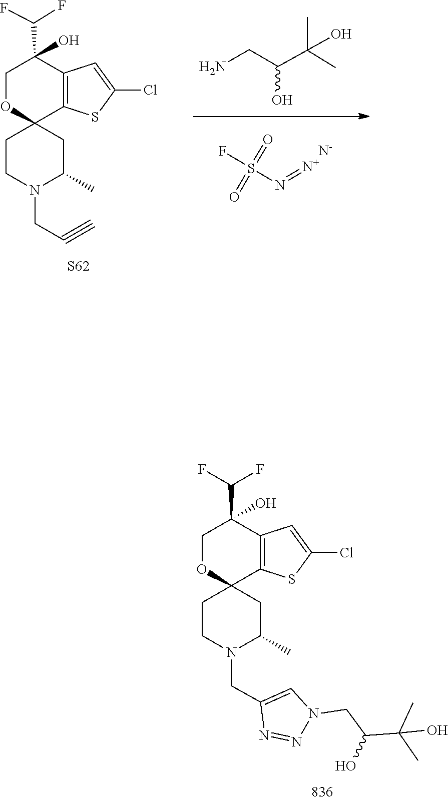

### Preparation of 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]triazol-1-yl]-3-methyl-butane-2,3-diol (836)

To a solution of 1-amino-3-methyl-butane-2,3-diol (approximately 29.64 mg, 0.2487 mmol) in DMSO (300.0 μL) was added aqueous hydrogen carbonate (Sodium salt) (approximately 250.0 μL of 1 M, 0.2500 mmol), and 550 uL fluorosulfonyl azide solution (˜0.45M in MTBE, ˜250 μmol). The resulting solution was stirred at room temperature for 20 minutes. A solution of (2′S,4R,7R)-2-chloro-4-(difluoromethyl)-2′-methyl-1′-prop-2-ynyl-spiro[5H-thieno[2,3-c]pyran-7,4′-piperidine]-4-ol S62 (30 mg, 0.08291 mmol) (30 mg) dissolved in DMSO (999.9 μL) was added followed by aqueous CuSO₄ (approximately 84.98 μL of 0.1 M, 0.008498 mmol), a solution of 4-[bis[4-hydroxy-1-(1H-triazol-4-yl)butyl]amino]-4-(1H-triazol-4-yl)butan-1-ol (approximately 84.98 μL of 0.1 M, 0.008498 mmol) in DMSO and aqueous sodium ascorbate (2R)-2-[(1S)-1,2-dihydroxyethyl]-4-hydroxy-5-oxo-2H-furan-3-olate (Sodium salt) (approximately 85.00 μL of 0.2 M, 0.01700 mmol). The reaction was heated at 50° C. overnight. Additional 100 uL 0.1M CuSO₄ and 100 uL 0.2M sodium ascorbate were added, and heating was continued overnight. The reaction was cooled to room temperature, diluted with water (2 mL) and DCM (1 mL), and stirred for several minutes. The mixture was passed through a parallel filtration plate and washed with DCM (1 mL). The organic layer was taken and evaporated. Purification by reversed-phase HPLC. Method: C18 Waters Sunfire column (30×150 mm, 5 micron) Gradient: MeCN in H₂O 10 mM Ammonium Hydroxide afforded 1-[4-[[(2′S,4R,7R)-2-chloro-4-(difluoromethyl)-4-hydroxy-2′-methyl-spiro[5H-thieno[2,3-c]pyran-7,4′-piperidine]-1′-yl]methyl]triazol-1-yl]-3-methyl-butane-2,3-diol (2.3, 5.5%) ¹H NMR (300 MHz, Methanol-d4) δ 8.91 (s, 1H), 7.91 (s, 1H), 6.85 (t, J=55.2 Hz, 1H), 5.72 (d, J 14.0 Hz, 1H), 5.24 (t, J=11.9 Hz, 1H), 5.08-4.41 (m, 5H), 3.65 (dd, J=35.8, 11.8 Hz, 3H), 3.33-2.70 (m, 3H), 2.50 (t, J=12.9 Hz, 1H), 2.35-1.97 (m, 9H). LCMS m/z 507.13 [M+1]⁺.

### Compounds 837-841

Compounds 837-841 (see Table 36) were prepared from click chemistry step as describes for compound 836 with the relevant amines. Amines were prepared by methods described above or obtained from commercial sources. Any modifications to methods are noted in Table 36 and accompanying footnotes.

TABLE 36
Method of preparation, structure and physicochemical data for compounds 837-841.

| Cmpd | Structure | Amine Reagent | Method | ¹H NMR |
|---|---|---|---|---|
| 837 | 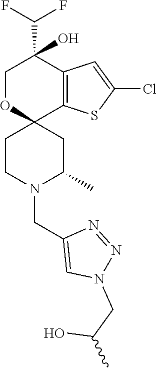 [SMILES: CC(O)Cn1cc(CN2CC[C@]3(C[C@@H]2C)OC[C@](O)(C(F)F)c2cc(Cl)sc23)nn1] | 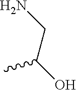 | Compound 836 | ¹H NMR (400 MHz, DMSO-d₆) δ 7.91 (d, J = 1.4 Hz, 1H), 7.02 (s, 1H), 6.19 (s, 1H), 5.98 (d, J = 55.1 Hz, 1H), 5.05 (d, J = 5.0 Hz, 1H), 4.34-4.16 (m, 2H), 4.02 (dd, J = 14.0, 8.3 Hz, 1H), 3.85 (d, J = 12.1 Hz, 1H), 3.84 (d, J = 14.5 Hz, 1H), 3.72 (d, J = 14.7 Hz, 1H),3.60 (d, J = 12.1 Hz, 1H), 2.62 (d, J = 11.5 Hz, 1H), 2.41 (q, J = 11.0, 10.1 Hz, 2H), 2.09 (d, J = 13.7 Hz, 1H), 1.86 (d, J = 13.4 Hz, 1H), 1.72 (td, J = 12.9, 4.4 Hz, 1H), 1.47-1.30 (m, 1H), 1.15 (d, J = 6.1 Hz, 3H), 1.04 (dd, J = 6.3, 4.0 Hz, 3H). LCMS m/z 463.11 [M + 1]⁺. |
| 838 | 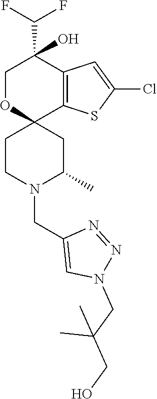 [SMILES: C[C@H]1C[C@@]2(CCN1Cc1cn(CC(C)(C)CO)nn1)OC[C@](O)(C(F)F)c1cc(Cl)sc12] | 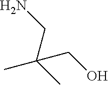 | Compound 836 | ¹H NMR (400 MHz, DMSO-d₆) δ 7.98 (s, 1H), 7.02 (s, 1H), 6.20 (s, 1H), 5.98 (t, J = 55.1 Hz, 1H), 5.14 (t, J = 5.5 Hz, 1H), 3.87 (d, J = 12.2Hz, 1H), 3.86 (d, J = 14.4 Hz, 1H), 3.66-3.59 (m, 4H), 2.63 (d, J = 11.5 Hz, 1H), 2.46- 2.33 (m, 2H), 2.10 (d, J = 14.0 Hz, 1H), 1.86 (d, J = 13.4 Hz, 1H), 1.72 (td, J = 13.0, 4.6 Hz, 1H), 1.54 (s, 6H), 1.36 (dd, J = 13.8, 11.2 Hz, 1H), 1.16 (d, J = |
| 837 | 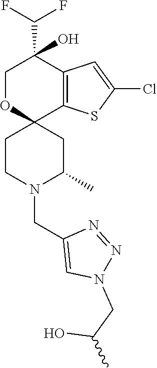 [SMILES: CC(O)Cn1cc(CN2CC[C@]3(C[C@@H]2C)OC[C@](O)(C(F)F)c2cc(Cl)sc23)nn1] | 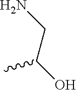 | Compound 836 | ¹H NMR (400 MHz, DMSO-d₆) δ 7.91 (d, J = 1.4 Hz, 1H), 7.02 (s, 1H), 6.19 (s, 1H), 5.98 (d, J = 55.1 Hz, 1H), 5.05 (d, J = 5.0 Hz, 1H), 4.34-4.16 (m, 2H), 4.02 (dd, J = 14.0, 8.3 Hz, 1H), 3.85 (d, J = 12.1 Hz, 1H), 3.84 (d, J = 14.5 Hz, 1H), 3.72 (d, J = 14.7 Hz, 1H),3.60 (d, J = 12.1 Hz, 1H), 2.62 (d, J = 11.5 Hz, 1H), 2.41 (q, J = 11.0, 10.1 Hz, 2H), 2.09 (d, J = 13.7 Hz, 1H), 1.86 (d, J = 13.4 Hz, 1H), 1.72 (td, J = 12.9, 4.4 Hz, 1H), 1.47-1.30 (m, 1H), 1.15 (d, J = 6.1 Hz, 3H), 1.04 (dd, J = 6.3, 4.0 Hz, 3H). LCMS m/z 463.11 [M + 1]⁺. |
| | | | | 6.2 Hz, 3H). LCMS m/z |
| | | | | 477.11 [M + 1]⁺. |
| 839 | 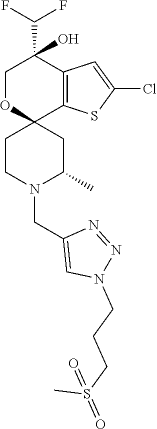 [SMILES: C[C@H]1C[C@@]2(CCN1Cc1cn(CCCS(C)(=O)=O)nn1)OC[C@](O)(C(F)F)c1cc(Cl)sc12] | 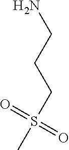 | Compound 836 | ¹H NMR (400 MHz, DMSO-d₆) δ 8.06 (s, 1H), 7.02 (s, 1H), 6.21 (s, 1H), 6.04 (t, J = 55.1 Hz, 1H), 4.48 (t, J = 7.0 Hz, 2H), 3.86 (d, J = 12.1 Hz, 1H), 3.85 (d, J = 14.6 Hz, 1H), 3.73 (d, J = 14.6 Hz, 1H), 3.60 (d, J = 12.3 Hz, 1H), 3.15-3.06 (m, 2H), 2.99 (s, 3H), 2.62 (d, J = 11.5 Hz, 1H), 2.47-2.19 (m, 4H), 2.10 (d, J = 13.8 Hz, 1H), 1.86 (d, J = 13.3 Hz, 1H), 1.73 (td, J = 13.0, 4.6 Hz, 1H), 1.35 (dd, J = 13.8, 11.3 Hz, 1H), 1.15 (d, J = 6.2 Hz, 3H). LCMS m/z 525.1 [M + 1]⁺. |
| 837 | 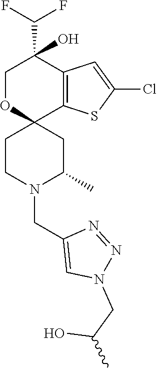 [SMILES: CC(O)Cn1cc(CN2CC[C@]3(C[C@@H]2C)OC[C@](O)(C(F)F)c2cc(Cl)sc23)nn1] | 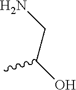 | Compound 836 | ¹H NMR (400 MHz, DMSO-d₆) δ 7.91 (d, J = 1.4 Hz, 1H), 7.02 (s, 1H), 6.19 (s, 1H), 5.98 (d, J = 55.1 Hz, 1H), 5.05 (d, J = 5.0 Hz, 1H), 4.34-4.16 (m, 2H), 4.02 (dd, J = 14.0, 8.3 Hz, 1H), 3.85 (d, J = 12.1 Hz, 1H), 3.84 (d, J = 14.5 Hz, 1H), 3.72 (d, J = 14.7 Hz, 1H), 3.60 (d, J = 12.1 Hz, 1H), 2.62 (d, J = 11.5 Hz, 1H), 2.41 (q, J = 11.0, 10.1 Hz, 2H), 2.09 (d, J = 13.7 Hz, 1H), 1.86 (d, J = 13.4 Hz, 1H), 1.72 (td, J = 12.9, 4.4 Hz, 1H), 1.47-1.30 (m, 1H), 1.15 (d, J = 6.1 Hz, 3H), 1.04 (dd, J = 6.3, 4.0 Hz, 3H). LCMS m/z 463.11 [M + 1]⁺. |
| 840 | 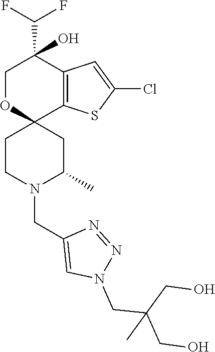 [SMILES: C[C@H]1C[C@@]2(CCN1Cc1cn(CC(C)(CO)CO)nn1)OC[C@](O)(C(F)F)c1cc(Cl)sc12] | 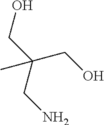 | Compound 836 | ¹H NMR (400 MHz, DMSO-d₆) δ 7.86 (s, 1H), 7.02 (s, 1H), 6.19 (s, 1H), 6.05 (t, J = 55.1 Hz, 1H), 4.74 (s, 2H), 4.26 (s, 2H), 3.88-3.79 (m, 2H), 3.76 (d, J = 14.6 Hz, 1H), 3.59 (d, J = 12.2 Hz, 1H), 3.21 (s, 4H), 2.59 (d, J = 12.0 Hz, 1H), 2.41 (dd, J = 23.7, 11.4 Hz, 2H), 2.08 (d, J = 13.8 Hz, 1H), 1.86 (d, J = 13.4 Hz, 1H), 1.71 (td, J = 13.0, 4.4 Hz, 1H), 1.36 (dd, J = 13.8, 11.3 Hz, 1H), 1.15 (d, J = 6.1 Hz, 3H), 0.65 (s, 3H). LCMS m/z 507.17 [M + 1]⁺. |
| 837 | 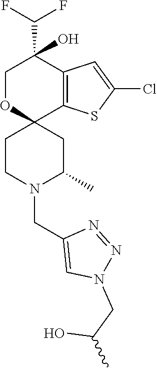 [SMILES: CC(O)Cn1cc(CN2CC[C@]3(C[C@@H]2C)OC[C@](O)(C(F)F)c2cc(Cl)sc23)nn1] | 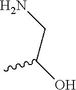 | Compound 836 | ¹H NMR (400 MHz, DMSO-d₆) δ 7.91 (d, J = 1.4 Hz, 1H), 7.02 (s, 1H), 6.19 (s, 1H), 5.98 (d, J = 55.1 Hz, 1H), 5.05 (d, J = 5.0 Hz, 1H), 4.34-4.16 (m, 2H), 4.02 (dd, J = 14.0, 8.3 Hz, 1H), 3.85 (d, J = 12.1 Hz, 1H), 3.84 (d, J = 14.5 Hz, 1H), 3.72 (d, J = 14.7 Hz, 1H), 3.60 (d, J = 12.1 Hz, 1H), 2.62 (d, J = 11.5 Hz, 1H), 2.41 (q, J = 11.0, 10.1 Hz, 2H), 2.09 (d, J = 13.7 Hz, 1H), 1.86 (d, J = 13.4 Hz, 1H), 1.72 (td, J = 12.9, 4.4 Hz, 1H), 1.47-1.30 (m, 1H), 1.15 (d, J = 6.1 Hz, 3H), 1.04 (dd, J = 6.3, 4.0 Hz, 3H). LCMS m/z 463.11 [M + 1]⁺. |
| 841 | 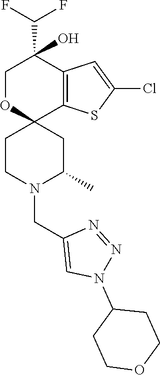 [SMILES: C[C@H]1C[C@@]2(CCN1Cc1cn(C3CCOCC3)nn1)OC[C@](O)(C(F)F)c1cc(Cl)sc12] | 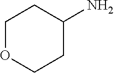 | Compound 836 | ¹H NMR (400 MHz, DMSO-d₆) δ 8.10 (s, 1H), 7.02 (s, 1H), 6.20 (s, 1H), 6.05 (t, J = 55.3 Hz, 1H), 4.72 (tt, J = 10.1, 4.9 Hz, 1H), 3.96 (d, J = 11.5 Hz, 2H), 3.86 (d, J = 12.1 Hz, 1H), 3.85 (d, J = 14.6 Hz, 1H), 3.69 (d, J = 14.6 Hz, 1H), 3.61 (d, J = 12.2 Hz, 1H), 3.49 (td, J = 11.5, 2.9 Hz, 2H), 2.62 (d, J = 11.7 Hz, 1H), 2.40 (q, J = 9.7, 7.3 Hz, 2H), 2.14-1.90 (m, 5H), 1.86 (d, J = 13.3 Hz, 1H), 1.72 (td, J = 12.9, 4.5 Hz, 1H), 1.36 (dd, J = 13.9, 11.3 Hz, 1H), 1.15 (d, J = 6.1 Hz, 3H). LCMS m/z 489.12 [M + 1]⁺. |

### Compound (842) [DIAST-1] and (843) [DIAST-2]

### (2′S, 7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](842) [DIAST-1] and (843) [DIAST-2]

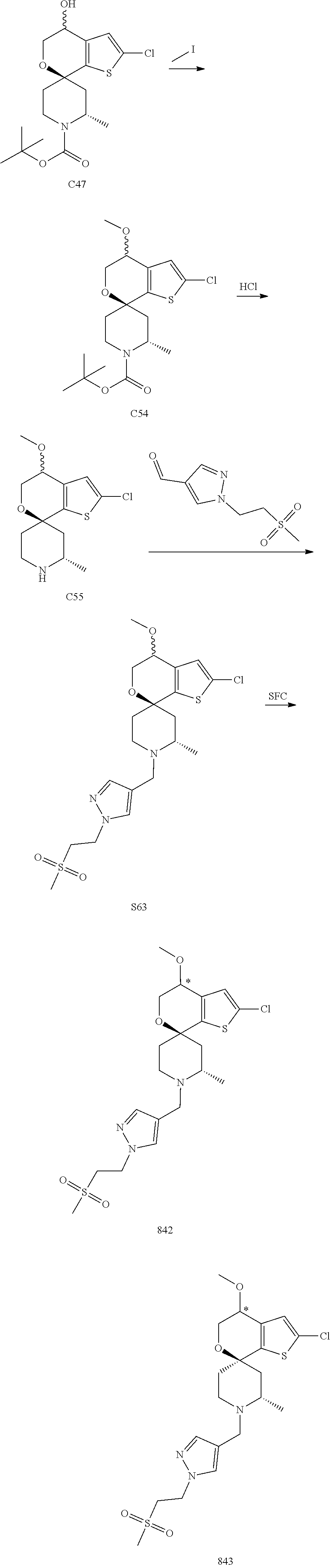

### Step 1. tert-butyl (2′S, 7R)-2-chloro-4-methoxy-2′-methyl-spiro[4,5-dihydrothieno[2,3-c]pyran-7,4′-piperidine]-1′-carboxylate (C54)

To a stirred tetrahydrofuran (2.5 mL) solution of tert-butyl (2′S,7R)-2-chloro-4-hydroxy-2′-methyl-spiro[4,5-dihydrothieno[2,3-c]pyran-7,4′-piperidine]-1′-carboxylate C47 (100 mg, 0.2675 mmol) was added NaH (43 mg of 60% w/w, 1.075 mmol) and iodomethane (151 mg, 1.064 mmol). The mixture was stirred at room temperature and left overnight. In the morning a saturated aqueous NH₄Cl solution was added to quench the reaction and the solution was extracted with DCM. The organics were washed with brine, dried with sodium sulfate and the solvent was removed through rotary evaporation to give tert-butyl (2′S,7R)-2-chloro-4-methoxy-2′-methyl-spiro[4,5-dihydrothieno[2,3-c]pyran-7,4′-piperidine]-1′-carboxylate C54. LCMS m/z 387.99 [M+1]⁺. The crude reaction mixture was telescoped directly to the next step.

### Step 2. (2′S, 7R)-2-chloro-4-methoxy-2′-methyl-spiro[4,5-dihydrothieno[2,3-c]pyran-7,4′-piperidine](C55)

To a solution of C₅₄ in Dioxane (500 μL) was added HCl (1.34 mL of 4 M, 5.360 mmol) in dioxane. The reaction stirred at room temperature for three hours. The solvent was removed in vacuo. Purification by reversed-phase HPLC. Method: C18 Waters Sunfire column (30×150 mm, 5 micron). Gradient: MeCN in H₂O with 0.1% trifluoroacetic acid afforded (2′S,7R)-2-chloro-4-methoxy-2′-methyl-spiro[4,5-dihydrothieno[2,3-c]pyran-7,4′-piperidine](Trifluoroacetate salt) (79.9 mg, 74%) LCMS m/z 288.1 [M+1]⁺.

### Step 3. (2′S, 7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](S63)

To a solution of (2′S,7R)-2-chloro-4-methoxy-2′-methyl-spiro[4,5-dihydrothieno[2,3-c]pyran-7,4′-piperidine](22 mg, 0.07644 mmol) and 1-(2-methylsulfonylethyl)pyrazole-4-carbaldehyde (23 mg, 0.1137 mmol) in dichloromethane (2 mL) in a 5 mL microwave vial was added acetic acid (22 μL, 0.3869 mmol) and polymer supported cyanoborohydride (114 mg of 2 mmol/g, 0.2280 mmol). The tube was capped and heated to 110° C. in a microwave reactor for an hour and a half Purification by reversed-phase HPLC. Method: C18 Waters Sunfire column (30×150 mm, 5 micron). Gradient: MeCN in H₂O with 0.1% trifluoroacetic acid afforded (2′S,7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](Trifluoroacetate salt) (18.9 mg, 41%) ¹H NMR (300 MHz, Methanol-d₄) δ 8.03-7.93 (m, 1H), 7.72 (s, 1H), 6.95 (s, 1H), 4.75-4.63 (m, 2H), 4.61-4.49 (m, 1H), 4.29 (d, J=14.1 Hz, 1H), 4.15 (dt, J=4.5, 2.6 Hz, 1H), 4.09-3.81 (m, 2H), 3.72 (t, J=6.2 Hz, 2H), 3.41 (d, J=0.6 Hz, 3H), 3.31 (m, 2H) 2.87 (dd, J=1.4, 0.8 Hz, 3H), 2.54-1.77 (m, 3H), 1.53 (dd, J=6.5, 2.2 Hz, 3H). LCMS m/z 474.11 [M+1]⁺.

### Step 3. (2′S, 7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](842) [DIAST-1] and (843) [DIAST-2]

Racemic mixture S63 (43 mg, 0.09071 mmol) was separated into constituent diastereomers by chiral SFC separation. (2′S,7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](Trifluoroacetate salt) (842) [DIAST-1](6 mg, 22%) ¹H NMR (400 MHz, Methanol-d₄) δ 7.75 (d, J=0.7 Hz, 1H), 7.55 (d, J=0.8 Hz, 1H), 6.88 (s, 1H), 4.63 (t, J=6.4 Hz, 2H), 4.09 (t, J=2.6 Hz, 1H), 4.02-3.56 (m, 6H), 3.39 (s, 3H), 2.90-2.49 (m, 6H), 2.17 (dt, J=14.3, 2.8 Hz, 1H), 2.00 (dt, J=13.9, 2.9 Hz, 1H), 1.79-1.59 (m, 2H), 1.26 (d, J=6.3 Hz, 3H).

(2′S,7R)-2-chloro-4-methoxy-2′-methyl-1′-[[1-(2-methylsulfonylethyl)pyrazol-4-yl]methyl]spiro[4,5-dihydrothieno[2,3-c]pyran-7,4′-piperidine](Trifluoroacetate salt) (843) [DIAST-2](12 mg, 45%)¹H NMR (400 MHz, Methanol-d₄) δ 7.72 (d, J=0.8 Hz, 1H), 7.53 (d, J=0.8 Hz, 1H), 6.87 (s, 1H), 4.62 (t, J=6.4 Hz, 2H), 4.11 (t, J=2.8 Hz, 1H), 4.00-3.65 (m, 6H), 3.39 (s, 3H), 2.87-2.47 (m, 6H), 2.13 (dt, J=14.3, 2.9 Hz, 1H), 2.09-1.81 (m, 2H), 1.45 (dd, J=14.3, 11.6 Hz, 1H), 1.22 (d, J=6.3 Hz, 3H).

### Compounds 844-846

Compounds 844-846 were prepared from the alkylation, deprotection and reductive amination steps as describes for intermediate S63 from intermediate C47 and the relevant alkyl halides. Alkyl halides were obtained from commercial sources. Any modifications to methods are noted in Table 37 and accompanying footnotes.

TABLE 37
Method of preparation, structure and physicochemical data for compounds 844-846.

| Cmpd | Structure | Alkyl Haldies | Method | ¹H NMR |
|---|---|---|---|---|
| 844 | 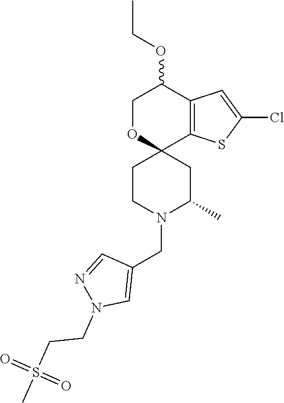 | 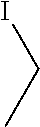 | Method S63^{1,2} | ¹H NMR (300 MHz, Methanol-d₄) δ 8.20- 7.96 (m, 1H), 7.72 (d, J = 0.7 Hz, 1H), 6.93 (s, 1H), 4.82-4.64 (m, 2H), 4.53 (d, J = 14.1 Hz, 1H), 4.37-4.18 (m, 2H), 4.15-3.84 (m, 2H), 3.82-3.55 (m, 4H), 3.51-3.35 (m, 1H), 3.22 (td, J = 13.0, 2.9 Hz, 1H), 2.87 (d, J = 1.5 Hz, 3H), 2.52-1.75 (m, 5H), 1.53 (dd, J = 6.5, 3.2 Hz, 3H), 1.20 (td, J = 7.0, 0.7 Hz, 3H). LCMS m/z 488.11 [M + 1]⁺ |
| 845 | 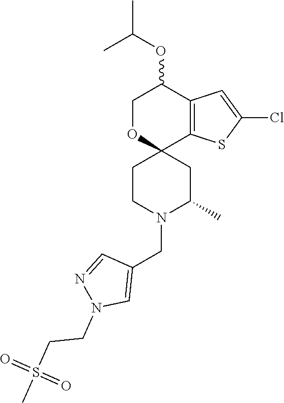 | 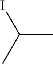 | Method S63^{1,2,3} | ¹H NMR (300 MHz, Methanol-d₄) δ 7.99 (s, 1H), 7.72 (s, 1H), 6.89 (s, 1H), 4.76- 4.59 (m, 2H), 4.53 (d, J = 14.1 Hz, 1H), 4.43- 4.18 (m, 2H), 4.08- 3.77 (m, 3H), 3.72 (t, J = 6.2 Hz, 2H), 3.42 (d, J = 37.3 Hz, 2H), 2.87 (d, J = 1.2 Hz, 3H), 2.65 (s, 1H), 2.53- 2.20 (m, 2H), 2.13- 1.70 (m, 2H), 1.53 (dd, J = 6.5, 3.2 Hz, 3H), 1.30-1.08 (m, 6H). LCMS m/z 502.1 [M + 1]+ |
| 846 | 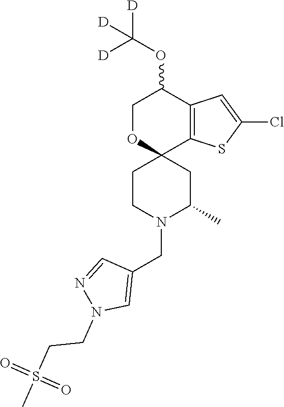 | 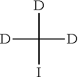 | Method S63^{1,2} | 1H NMR (300 MHz, Methanol-d₄) δ 8.01 (d, J = 1.4 Hz, 1H), 7.74 (s, 1H), 6.97 (s, 1H), 4.78-4.67 (m, 2H), 4.55 (d, J = 14.1 Hz, 1H), 4.31 (d, J = 14.1 Hz, 1H), 4.17 (dt, J = 4.5, 2.6 Hz, 1H), 4.13-3.88 (m, 2H), 3.74 (t, J = 6.2 Hz, 2H), 3.31 (m, 2H) 2.89 (dd, J = 1.4, 0.7 Hz, 3H), 2.60-1.75 (m, 4H), 1.55 (dd, J = 6.5, 2.2 Hz, 3H). LCMS m/z 477.11 [M + 1]⁺ |

Footnotes
¹Purification by silica gel chromatography (Gradient: 0-40% EtOAc in heptane) yielded the product (Step 1)
²Deprotected product was not purified before telescoping to the reductive amination step (Step 2).
³Reaction was heated to 60° C. for three days.

### Preparation of Compound 847

### [(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]acetate (847)

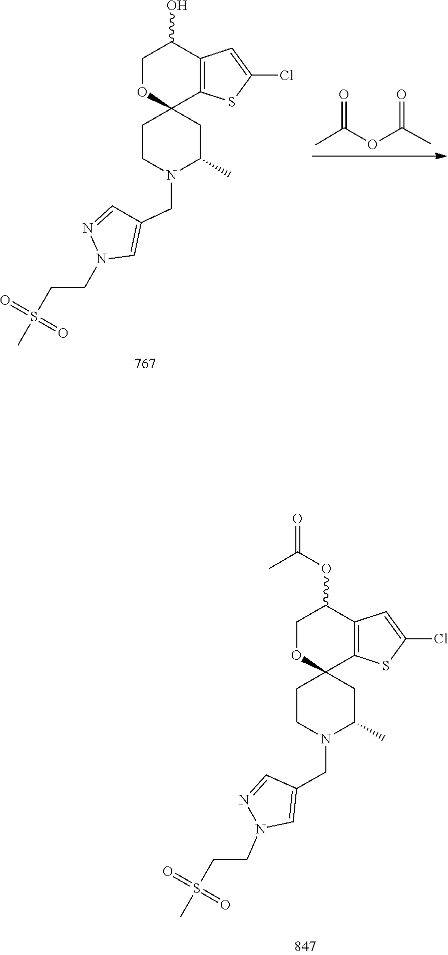

### [(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]acetate (847)

To a solution of (2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-ol 767 (45 mg, 0.09782 mmol) in Pyridine (1.4 mL) was added Acetic Anhydride (1.4 mL, 14.84 mmol) and the reaction was stirred at rt. The solvents were removed in vacuo. Purification by silica gel chromatography (Gradient: 0-20% MeOH in DCM) yielded the product [(2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]acetate (29.1 mg, 59%) ¹H NMR (300 MHz, Methanol-d₄) δ 7.87-7.68 (m, 1H), 7.55 (t, J=0.9 Hz, 1H), 6.84 (d, J=1.2 Hz, 1H), 5.52-5.47 (m, 1H), 4.63 (t, J=6.4 Hz, 2H), 4.14-3.56 (m, 6H), 2.93-2.41 (m, 6H), 2.36-2.10 (m, 1H), 2.06-1.38 (m, 6H), 1.26 (dd, J=6.3, 4.0 Hz, 3H). LCMS m/z 502.11 [M+1]⁺.

### Preparation 848

### (2′S, 7R)-2-chloro-2′,4-dimethyl-1′-[(1-methylpyrazol-4-yl)methyl]spiro[5H-thieno[2,3-c]pyran-7,4′-piperidine]-4-ol (848)

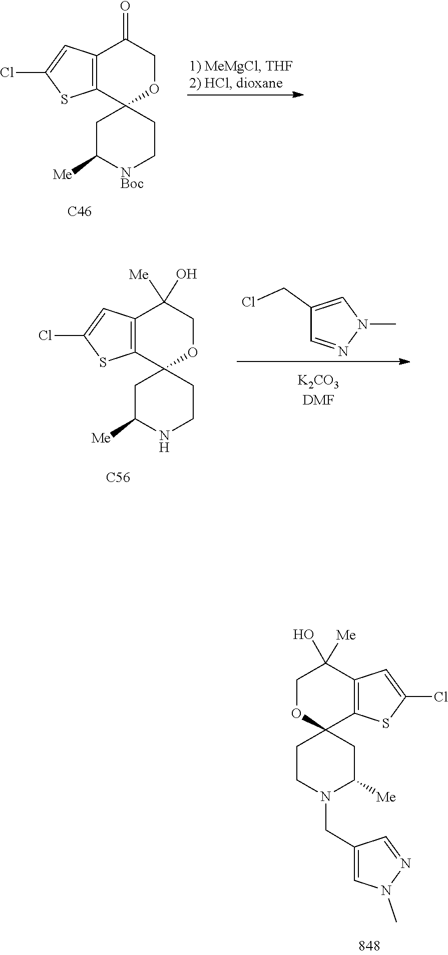

### Step 1. Synthesis of (2′S, 7R)-2-chloro-2′,4-dimethyl-spiro[5H-thieno[2,3-c]pyran-7,4′-piperidine]-4-ol (C56)

To a solution of tert-butyl (2S,4R)-2′-chloro-2-methyl-4′-oxo-spiro[piperidine-4,7′-thieno[2,3-c]pyran]-1-carboxylate C46 (107 mg, 0.288 mmol) in THF (2.7 mL) was added 3.0 M MeMgCl in THF (191 μL, 0.573 mmol) at 0° C. After 20 min, the reaction was quenched with sat. aq. ammonium chloride and extracted with EtOAc. The organic layer was washed with brine, dried over anhydrous sodium sulfate, filtered and concentrated in vacuo. Purification by silica gel chromatography (0 to 60% EtOAc in hexanes) yielded a material that was dissolved in dioxane (2.7 mL) and 4 M HCl in dioxane (720 μL, 2.88 mmol). After 4 h, the crude mixture was concentrated and taken forward to the next step without any further purification (28 mg, 20% yield). LCMS m/z 288.03 [M+H]⁺.

### Step 2. Synthesis of (2′S, 7R)-2-chloro-2′,4-dimethyl-1′-[(1-methylpyrazol-4-yl)methyl]spiro[5H-thieno[2,3-c]pyran-7,4′-piperidine]-4-ol (848)

To a solution of (2′S,7R)-2-chloro-2′,4-dimethyl-spiro[5H-thieno[2,3-c]pyran-7,4′-piperidine]-4-ol C₅₆ (28 mg, 0.097 mmol) in DMF (420 μL) was added 4-(chloromethyl)-1-methyl-pyrazole (15 mg, 0.11 mmol) and K₂CO₃ (40 mg, 0.29 mmol). The reaction was stirred at 60° C. for 3 h and then quenched with water and DCM. The organic layer was separated and concentrated in vacuo. Purification by silica gel chromatography (0 to 20% MeOH in DCM) yielded (2′S,7R)-2-chloro-2′,4-dimethyl-1′-[(1-methylpyrazol-4-yl)methyl]spiro[5H-thieno[2,3-c]pyran-7,4′-piperidine]-4-ol (8.7 mg, 46% yield) as a 60:40 ratio of diastereomers. ¹H NMR (300 MHz, Methanol-d₄) δ 7.57 (s, 1H), 7.43 (d, J=0.8 Hz, 1H), 6.90 (d, J=1.5 Hz, 1H), 3.91-3.77 (m, 4H), 3.67-3.53 (m, 3H), 2.79-2.45 (m, 3H), 2.19-1.94 (m, 2H), 1.87-1.67 (m, 1H), 1.55 (ddd, J=13.3, 11.5, 1.6 Hz, 1H), 1.38 (d, J=3.8 Hz, 3H), 1.22 (dd, J=6.3, 2.1 Hz, 3H). LCMS m/z 382.09 [M+H]⁺.

### Preparation 849 and 850

### (2′S,7R)-2-chloro-2′-methyl-1′-[(1-methylpyrazol-4-yl)methyl]-4-phenyl-spiro[5H-thieno[2,3-c]pyran-7,4′-piperidine]-4-ol (Trifluoroacetate salt) (849 and 850)

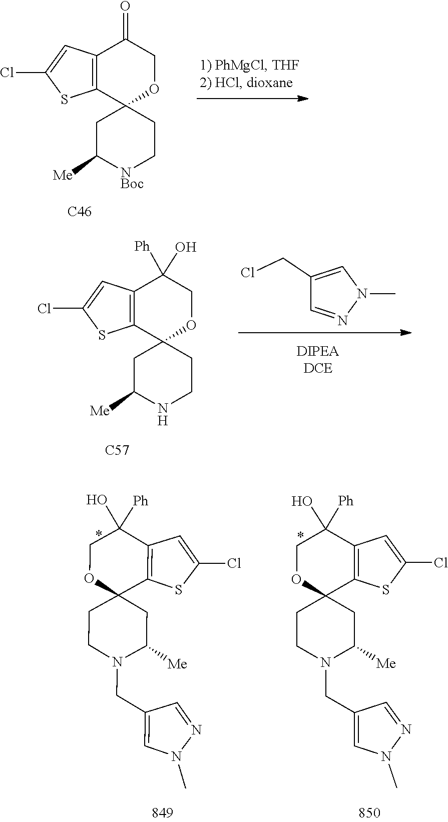

### Step 1. Synthesis of (2′S, 7R)-2-chloro-2′-methyl-4-phenyl-spiro[5H-thieno[2,3-c]pyran-7,4′-piperidine]-4-ol (C57)

To a solution of tert-butyl (2S,4R)-2′-chloro-2-methyl-4′-oxo-spiro[piperidine-4,7′-thieno[2,3-c]pyran]-1-carboxylate C46 (158 mg, 0.425 mmol) in THF (4 mL) was added 2.0 M PhMgCl in THF (420 μL, 0.84 mmol) at 0° C. After 2 h, the reaction was quenched with sat. aq. ammonium chloride and extracted with EtOAc. The organic layer was washed with brine, dried over anhydrous sodium sulfate, filtered and concentrated in vacuo. Purification by silica gel chromatography (0 to 60% EtOAc in hexanes) yielded two pure diastereomeric product fractions. Each fraction was dissolved in dioxane (2 mL) and 4 M HCl in dioxane (106 μL, 0.42 mmol). After stirring overnight, the reaction was quenched with sat. aq. sodium bicarbonate and extracted with DCM. The crude mixture was concentrated and taken forward to the next step without any further purification (C57, 25.3 mg, 74% yield). LCMS m/z 349.99 [M+H]⁺.

### Step 2. Synthesis of (2′S, 7R)-2-chloro-2′-methyl-1′-[(1-methylpyrazol-4-yl)methyl]-4-phenyl-spiro[5H-thieno[2,3-c]pyran-7,4′-piperidine]-4-ol (Trifluoroacetate salt) (849 and 850)

To a solution of (2′S,7R)-2-chloro-2′-methyl-4-phenyl-spiro[5H-thieno[2,3-c]pyran-7,4′-piperidine]-4-ol C57 (20 mg, 0.044 mmol) in DCE (540 μL) was added 4-(chloromethyl)-1-methyl-pyrazole (6.9 mg, 0.053 mmol) and DIPEA (17 mg, 0.13 mmol). The reaction was stirred at 60° C. for 3 h and then quenched with water and DCM. The organic layer was separated and concentrated in vacuo. Purification by silica gel chromatography (0 to 20% MeOH in DCM) followed by purification by reversed-phase HPLC (Method: C18 Waters Sunfire column (30×150 mm, 5 micron). Gradient: MeCN in H₂O with 0.1% trifluoroacetic acid) yielded the products:

Compound 849 [DIAST 1]: (3 mg, 12% yield). ¹H NMR (300 MHz, DMSO-d₆) δ 9.73 (s, 1H), 7.91 (d, J=3.7 Hz, 1H), 7.59 (d, J=3.5 Hz, 1H), 7.45-7.09 (m, 5H), 6.71 (d, J=7.0 Hz, 1H), 4.42 (d, J=13.8 Hz, 1H), 4.30-4.08 (m, 1H), 3.39-2.78 (m, 3H), 2.48-2.15 (m, 2H), 1.99 (tt, J=26.9, 13.1 Hz, 2H), 1.44 (dd, J=10.5, 6.3 Hz, 3H). LCMS m/z 444.12 [M+H]⁺. [Some proton peaks behind solvent].

Compound 850 [DIAST 2]: (7 mg, 28% yield). ¹H NMR (300 MHz, DMSO-d₆) δ 7.91 (d, J=3.6 Hz, 1H), 7.59 (d, J=3.5 Hz, 1H), 7.47-7.09 (m, 5H), 6.71 (d, J=7.2 Hz, 1H), 4.43 (d, J=13.9 Hz, 1H), 4.19 (dd, J=14.0, 5.1 Hz, 1H), 3.96-3.80 (m, 6H), 3.30 (t, J=13.5 Hz, 2H), 3.02 (d, J=16.3 Hz, 1H), 2.44-2.20 (m, 1H), 1.96 (td, J=29.4, 26.8, 16.1 Hz, 2H), 1.44 (dd, J=10.5, 6.3 Hz, 3H). LCMS m/z 444.28 [M+H]⁺.

### Preparation 851 and 852

### (2′S, 7R)-2-chloro-2′-methyl-1′-[[1-(2-methylsulfonylethyl)pyrazol-4-yl]methyl]-4-(trifluoromethyl)spiro[5H-thieno[2,3-c]pyran-7,4′-piperidine]-4-ol (851 and 852)

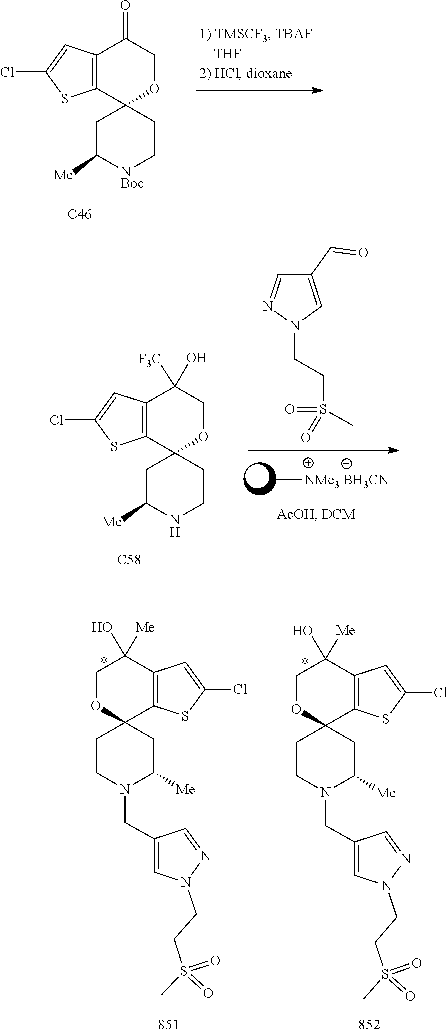

### Step 1. Synthesis of (2′S, 7R)-2-chloro-2′-methyl-4-(trifluoromethyl)spiro[5H-thieno[2,3-c]pyran-7,4′-piperidine]-4-ol (C58)

To a solution of tert-butyl (2S,4R)-2′-chloro-2-methyl-4′-oxo-spiro[piperidine-4,7′-thieno[2,3-c]pyran]-1-carboxylate 46 (128 mg, 0.344 mmol), TMSCF₃ (62 μL, 0.42 mmol) and THF (3.5 mL) at 0° C. was added 1 M TBAF in THF (35 μL, 0.035 mmol). After stirring for 30 min at room temperature, an additional portion of 1 M TBAF in THF (172 μL, 0.172 mmol) was added. The reaction was quenched with water and DCM. The organic layer was separated and concentrated in vacuo. Purification by silica gel chromatography (0 to 30% EtOAc in hexanes) yielded two pure diastereomeric product fractions. Each fraction was dissolved in dioxane (1 mL) and 4 M HCl in dioxane (1 mL, 4 mmol). After 1 h, the crude mixture was concentrated and taken forward to the next step without any further purification (C58: 63.5 mg, 54% yield). LCMS m/z 341.93 [M+H]⁺.

### Step 2. Synthesis of (2′S, 7R)-2-chloro-2′-methyl-1′-[[1-(2-methylsulfonylethyl)pyrazol-4-yl]methyl]-4-(trifluoromethyl)spiro[5H-thieno[2,3-c]pyran-7,4′-piperidine]-4-ol (851 and 852)

To a microwave tube containing a solution of (2′S)-2-chloro-2′-methyl-4-(trifluoromethyl)spiro[5H-thieno[2,3-c]pyran-7,4′-piperidine]-4-ol C₅₈ (63.0 mg, 0.175 mmol), 1-(2-methylsulfonylethyl)pyrazole-4-carbaldehyde (53.5 mg, 0.26 mmol), AcOH (52.4 mg, 0.87 mmol) and DCM (3.3 mL) was added cyanoborohydride on Amberlite resin, ˜2 mmol/g (262 mg). The tube was sealed and heated at 110° C. in a microwave reactor for 45 min. The reaction was filtered and the recovered resin was stirred vigorously in DCM to collect any additional product. The combined organic layers were concentrated in vacuo. Purification by silica gel chromatography (0 to 20% MeOH in DCM) yielded the products:

Compound 851 [DIAST 1]: (2′S,7R)-2-chloro-2′-methyl-1′-[[1-(2-methylsulfonylethyl)pyrazol-4-yl]methyl]-4-(trifluoromethyl)spiro[5H-thieno[2,3-c]pyran-7,4′-piperidine]-4-ol (59.1 mg, 62% yield). ¹H NMR (300 MHz, CDCl₃) δ 7.42 (d, J=10.0 Hz, 2H), 6.93 (d, J=1.1 Hz, 1H), 4.54 (td, J=5.9, 3.2 Hz, 2H), 4.09 (d, J=12.4 Hz, 1H), 3.90-3.41 (m, 5H), 2.73-2.34 (m, 6H), 2.08-1.89 (m, 2H), 1.80 (td, J=12.9, 4.2 Hz, 1H), 1.59 (dd, J=14.2, 11.4 Hz, 1H), 1.18 (d, J=6.2 Hz, 3H); and

Compound 852 [DIAST 2]: (2′S,7R)-2-chloro-2′-methyl-1′-[[1-(2-methylsulfonylethyl)pyrazol-4-yl]methyl]-4-(trifluoromethyl)spiro[5H-thieno[2,3-c]pyran-7,4′-piperidine]-4-ol (23 mg, 63% yield). ¹H NMR (300 MHz, Methanol-d₄) δ 7.82-7.68 (m, 1H), 7.53 (d, J=0.8 Hz, 1H), 6.96 (d, J=1.0 Hz, 1H), 4.63 (t, J=6.4 Hz, 2H), 4.05 (d, J=12.5 Hz, 1H), 3.92-3.57 (m, 5H), 2.90-2.45 (m, 6H), 2.17 (dt, J=14.2, 2.8 Hz, 1H), 2.02-1.76 (m, 2H), 1.54 (dd, J=14.2, 11.5 Hz, 1H), 1.23 (d, J=6.3 Hz, 3H). LCMS m/z 527.99 [M+H]⁺.

### Preparation 853, 854, and 855

### N-[(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]acetamide (853, 854, 855)

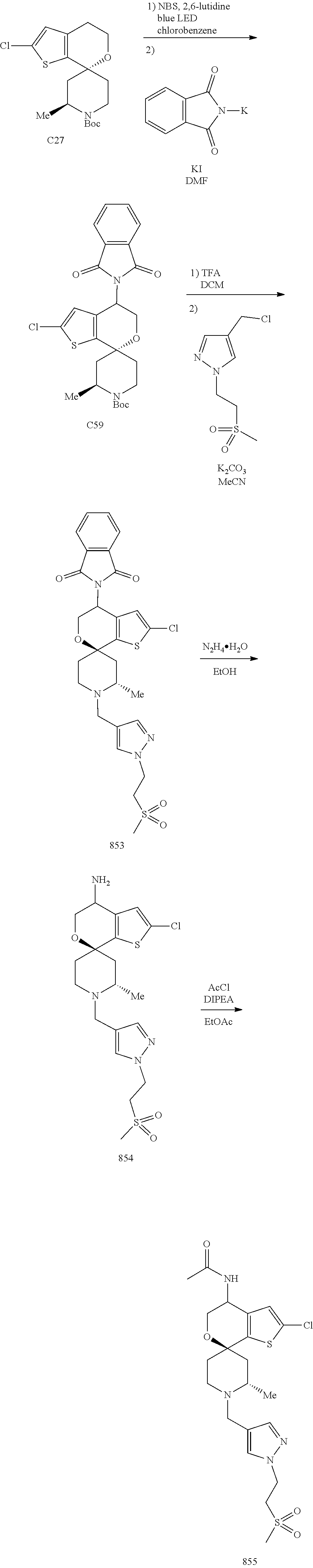

### Step 1. Synthesis of tert-butyl (2′S, 7R)-2-chloro-4-(1,3-dioxoisoindolin-2-yl)-2′-methyl-spiro[4,5-dihydrothieno[2,3-c]pyran-7,4′-piperidine]-1′-carboxylate (C59)

To a mixture of tert-butyl (2′S,7R)-2-chloro-2′-methyl-spiro[4,5-dihydrothieno[2,3-c]pyran-7,4′-piperidine]-1′-carboxylate C27 (1.12 g, 3.07 mmol) and 2,6-lutidine (0.6 mL, 5.2 mmol) in chlorobenzene (20 mL) was added NBS (875 mg, 4.9 mmol). The reaction was irradiated under a blue LED for 5 min. The reaction was filtered and rinsed with EtOAc. The filtrate was diluted with EtOAc and washed with water and brine. The organic layer was dried over anhydrous magnesium sulfate, filtered and concentrated in vacuo. To the crude material was added phthalimide potassium salt (600 mg, 3.2 mmol), KI (50 mg, 0.3 mmol), and DMF (10 mL). The reaction was stirred at 100° C. for 15 min. The reaction was cooled to room temperature and diluted with water and diethyl ether. The aqueous layer was extracted with diethyl ether. The combined organic layers were washed with water and brine and concentrated in vacuo. Purification by silica gel chromatography (0 to 50% EtOAc in heptane) yielded the product as a clear oil (362 mg, 17% yield). ¹H NMR (400 MHz, CDCl₃) δ 7.83-7.73 (m, 2H), 7.72-7.63 (m, 2H), 6.39 (d, J=9.1 Hz, 1H), 5.34-5.23 (m, 1H), 4.19 (ddd, J=28.2, 11.5, 7.9 Hz, 1H), 3.99-3.80 (m, 2H), 3.76-3.57 (m, 1H), 3.28 (tdd, J=14.4, 8.8, 5.4 Hz, 1H), 2.58-2.37 (m, 1H), 2.19-1.99 (m, 1H), 1.86-1.74 (m, 1H), 1.65 (ddd, J=24.2, 14.4, 10.9 Hz, 1H), 1.40 (d, J=0.7 Hz, 9H), 1.26-1.21 (m, 3H). LCMS m/z 501.88 [M+H]⁺.

### Step 2. Synthesis of 2-[(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]isoindoline-1,3-dione (853)

To a solution of tert-butyl (2′S,7R)-2-chloro-4-(1,3-dioxoisoindolin-2-yl)-2′-methyl-spiro[4,5-dihydrothieno[2,3-c]pyran-7,4′-piperidine]-1′-carboxylate C59 (132 mg, 0.23 mmol) in DCM (2 mL) was added TFA (175 μL, 2.3 mmol). The solution was stirred at room temperature for 20 min. The solution was concentrated in vacuo, redissolved in DCM, and concentrated again. The crude oil was dissolved in MeCN (2 mL) and treated with K₂CO₃ (100 mg, 0.72 mmol) followed by 4-(chloromethyl)-1-(2-methylsulfonylethyl)pyrazole (hydrochloride salt) 853 (100 mg, 0.39 mmol). The reaction was stirred at 70° C. for 2 h. The mixture was cooled to room temperature and diluted with water and EtOAc. The aqueous layer was removed and the organic layer was washed with water and brine. The organic layer was dried over anhydrous sodium sulfate, filtered and concentrated in vacuo. Purification by silica gel chromatography (0 to 50% EtOAc in heptane) yielded the product as a clear oil (118 mg, 71% yield). ¹H NMR (400 MHz, CDCl₃) δ 7.95-7.82 (m, 2H), 7.82-7.70 (m, 2H), 7.55 (s, 2H), 6.49 (d, J=5.7 Hz, 1H), 5.37 (dt, J=13.3, 6.9 Hz, 1H), 4.62 (s, 2H), 4.26 (dt, J=12.2, 6.7 Hz, 1H), 4.03-3.78 (m, 2H), 3.66 (d, J=6.0 Hz, 3H), 2.60 (d, J=91.0 Hz, 6H), 2.05 (d, J=17.3 Hz, 3H), 1.32 (d, J=37.8 Hz, 4H). LCMS m/z 588.98 [M+H]⁺.

### Step 3. Synthesis of (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-amine (854)

To a mixture of 2-[(2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]isoindoline-1,3-dione 853 (100 mg, 0.16 mmol) in EtOH (5 mL) was added hydrazine monohydrate (76 μL, 1.6 mmol). The reaction was stirred at 80° C. After 1 h, the reaction was cooled to 0° C., filtered, and rinsed with ice-cold EtOH. The filtrate was concentrated in vacuo. Purification by reversed-phase chromatography (Column: C18. Gradient: 10-100% MeCN in water with 0.1% trifluoroacetic acid) afforded the product as a TFA salt. The purified material was treated with sat. aq. sodium bicarbonate and DCM. The aqueous layer was extracted with DCM and the combined organic layers were concentrated in vacuo to deliver the product as a clear oil (40 mg, 55% yield). ¹H NMR (400 MHz, CDCl₃) δ 7.43 (d, J=0.7 Hz, 1H), 7.38 (s, 1H), 6.69 (s, 1H), 4.52 (dd, J=6.7, 5.2 Hz, 2H), 3.82 (dd, J=11.7, 3.6 Hz, 1H), 3.74 (d, J=14.4 Hz, 1H), 3.63-3.44 (m, 5H), 2.62-2.32 (m, 6H), 1.97-1.87 (m, 2H), 1.68 (td, J=13.2, 4.5 Hz, 1H), 1.56 (dd, J=13.6, 11.3 Hz, 1H), 1.13 (d, J=6.2 Hz, 3H). LCMS m/z 459.03 [M+H]⁺.

### Step 4. Synthesis of N-[(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]acetamide (855)

To a mixture of (2′S,7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-amine 854 (32 mg, 0.070 mmol) and DIPEA (25 μL, 0.14 mmol) in EtOAc (1 mL) was added acetyl chloride (8 μL, 0.11 mmol). The reaction was stirred at room temperature for 1 h and then quenched by addition of sat. aq. ammonium chloride. The aqueous layer was removed and the organic layer was dried over anhydrous sodium sulfate, filtered and concentrated in vacuo. Purification by silica gel chromatography (0 to 20% MeOH in DCM) yielded the product as a white foam (11 mg, 30% yield). ¹H NMR (400 MHz, CDCl₃) δ 7.43 (d, J=0.8 Hz, 1H), 7.39 (s, 1H), 6.67 (d, J=1.5 Hz, 1H), 5.77 (t, J=7.9 Hz, 1H), 4.82-4.70 (m, 1H), 4.52 (t, J=6.0 Hz, 2H), 3.91-3.65 (m, 3H), 3.57 (td, J=6.0, 2.1 Hz, 2H), 3.50 (d, J=14.4 Hz, 1H), 2.68-2.43 (m, 2H), 2.43-2.26 (m, 4H), 2.10-1.39 (m, 7H), 1.14 (t, J=6.2 Hz, 3H). LCMS m/z 501.03 [M+H]⁺.

### Preparation 856

### (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-carbonitrile (Trifluoroacetate salt) (856)

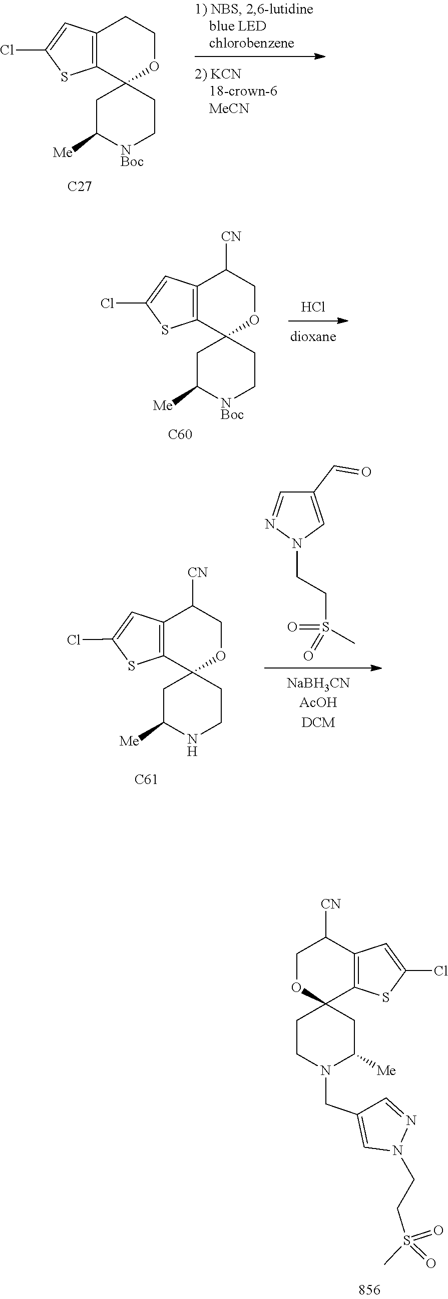

### Step 1. Synthesis of tert-butyl (2′S)-2-chloro-4-cyano-2′-methyl-spiro[4,5-dihydrothieno[2,3-c]pyran-7,4′-piperidine]-1′-carboxylate (C60)

To a mixture of tert-butyl (2′S)-2-chloro-2′-methyl-spiro[4,5-dihydrothieno[2,3-c]pyran-7,4′-piperidine]-1′-carboxylate C27 (1 g, 2.8 mmol) and 2,6-lutidine (536 mg, 5.0 mmol) in chlorobenzene (28 mL) was added NBS (795 mg, 4.5 mmol). The reaction was irradiated under a blue LED for 30 min. The reaction was quenched with sodium bisulfite and extracted with DCM. The combined organic layers were washed with brine and concentrated in vacuo. The brominated crude material was combined with KCN (127 mg, 2.0 mmol) and 18-crown-6 (516 mg, 2.0 mmol) in MeCN (10 mL) and was stirred at 60° C. overnight. The reaction was cooled to room temperature and diluted with EtOAc. The reaction was washed with water and sat. aq. sodium bicarbonate. The organic layer was dried over anhydrous sodium sulfate, filtered and concentrated in vacuo. Purification by silica gel chromatography (0 to 40% EtOAc in hexanes) yielded the product (245 mg, 23% yield). LCMS m/z 382.72 [M+H]⁺.

### Step 2. Synthesis of (2′S)-2-chloro-2′-methyl-spiro[4,5-dihydrothieno[2,3-c]pyran-7,4′-piperidine]-4-carbonitrile (Hydrochloride salt) (C61)

A solution of tert-butyl (2′S)-2-chloro-4-cyano-2′-methyl-spiro[4,5-dihydrothieno[2,3-c]pyran-7,4′-piperidine]-1′-carboxylate C60 (202 mg, 0.53 mmol) and 4 M HCl in dioxane (0.5 mL, 2.0 mmol) was stirred at room temperature for 4 h. The reaction was concentrated in vacuo, redissolved in MeOH, and then concentrated again. Purification by reversed-phase chromatography (Column: C18. Gradient: 10-90% MeCN in water with hydrochloric acid) afforded the product (31.9 mg, 18% yield). LCMS m/z 283.06 [M+H]⁺.

### Step 3. Synthesis of (2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-carbonitrile (Trifluoroacetate salt) (856)

To a solution of 1-(2-methylsulfonylethyl)pyrazole-4-carbaldehyde C61 (7.9 mg, 0.028 mmol) and 1-(2-methylsulfonylethyl)pyrazole-4-carbaldehyde (8.5 mg, 0.042 mmol) in DCM (2 mL) was added AcOH (8 μL, 0.14 mmol) followed by solid-supported NaBH₃CN (0.084 mmol). The reaction was heated to 110° C. in a microwave reactor. After 1 h, the reaction was cooled, filtered and concentrated in vacuo. Purification by reversed-phase HPLC (Method: C18 Waters Sunfire column (30×150 mm, 5 micron). Gradient: MeCN in H₂O with 0.1% trifluoroacetic acid) yielded the product (5.1 mg, 31% yield). LCMS m/z 468.94 [M+H]⁺.

### Preparation 857

### N-[(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]-N-methyl-acetamide (857)

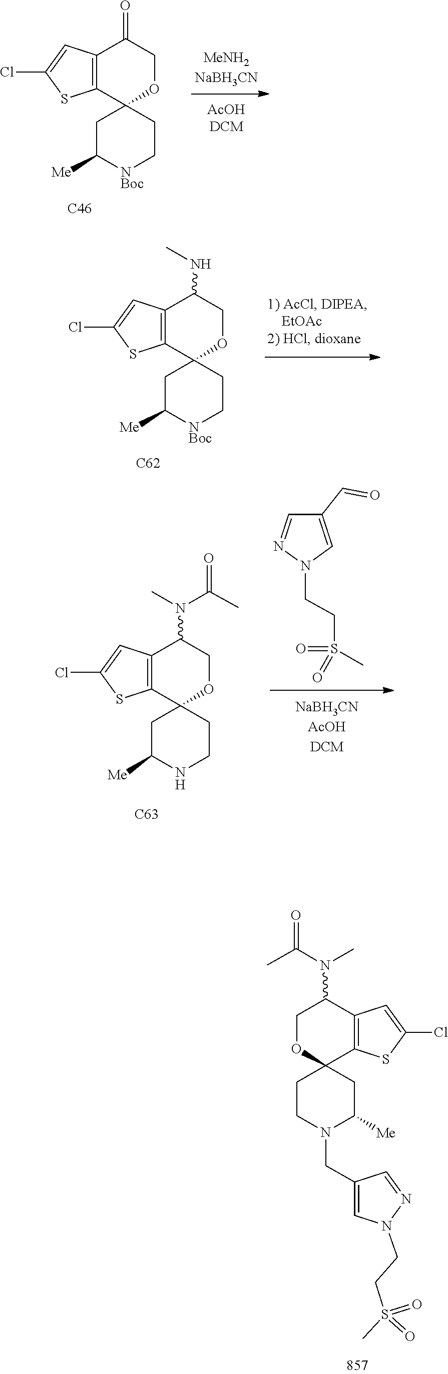

### Step 1. Synthesis of tert-butyl (2′S, 7R)-2-chloro-2′-methyl-4-(methylamino)spiro[4,5-dihydrothieno[2,3-c]pyran-7,4′-piperidine]-1′-carboxylate (C62)

A mixture of tert-butyl (2S,4R)-2′-chloro-2-methyl-4′-oxo-spiro[piperidine-4,7′-thieno[2,3-c]pyran]-1-carboxylate C46 (200 mg, 0.54 mmol), 2 M MeNH₂ (1 mL, 2 mmol), and solid-supported NaBH₃CN (1 mmol) in DCM (3 mL) was stirred at 90° C. in a microwave reactor. After 2 h, AcOH (40 μL, 0.70 mmol) was added. After 3 h, the reaction was cooled, filtered and concentrated in vacuo. Purification by reversed-phase chromatography (Column: C18. Gradient: 10-100% MeCN in water with 0.1% trifluoroacetic acid) afforded the product. The isolated product was stirred in sat. aq. sodium bicarbonate and DCM. The organic layer was separated and concentrated in vacuo to give the product as a pale yellow oil (112 mg, 52% yield). ¹H NMR (400 MHz, CDCl₃) δ 6.67 (d, J=7.8 Hz, 1H), 4.01-3.75 (m, 3H), 3.70-3.59 (m, 1H), 3.33-3.19 (m, 2H), 2.42 (d, J=1.4 Hz, 3H), 2.27-1.92 (m, 3H), 1.82-1.54 (m, 2H), 1.41 (s, 9H), 1.19 (d, J=6.6 Hz, 3H). LCMS m/z 386.96 [M+H]⁺.

### Step 2. Synthesis of N-[(2′S, 7R)-2-chloro-2′-methyl-spiro[4,5-dihydrothieno[2,3-c]pyran-7,4′-piperidine]-4-yl]-N-methyl-acetamide (Hydrochloride salt) (C63)

To a mixture of tert-butyl (2′S,7R)-2-chloro-2′-methyl-4-(methylamino)spiro[4,5-dihydrothieno[2,3-c]pyran-7,4′-piperidine]-1′-carboxylate C62 (105 mg, 0.26 mmol) and DIPEA (0.1 mL, 0.57 mmol) in EtOAc (2 mL) was added acetyl chloride (20 μL, 0.28 mmol). The reaction was stirred for 1 h and then concentrated in vacuo. To the crude material was added DCM and 1 M HCl. The aqueous layer was removed and the organic layer was washed with 1 M HCl. The organic layer was separated and concentrated in vacuo. Purification by silica gel chromatography (0 to 100% EtOAc in heptane) yielded the acetylated product as a clear oil (95 mg, 85% yield). ¹H NMR (400 MHz, CDCl₃) δ 6.64 (s, 1H), 5.53 (ddd, J=10.8, 4.1, 1.9 Hz, 1H), 4.10-3.98 (m, 2H), 3.96-3.68 (m, 3H), 3.34 (ddt, J=17.1, 8.6, 4.5 Hz, 1H), 2.87 (d, J=3.7 Hz, 3H), 2.24 (h, J=8.1 Hz, 2H), 2.16 (s, 3H), 2.05-1.91 (m, 1H), 1.78 (dd, J=13.9, 10.9 Hz, 1H), 1.58 (dd, J=14.6, 11.0 Hz, 1H), 1.50 (s, 9H), 1.30-1.25 (m, 3H). LCMS m/z 429.05 [M+H]⁺.

The acetylated product was stirred in 4 M HCl in dioxane (250 μL, 1 mmol) overnight. The reaction was concentrated in vacuo and the deprotected product was crystallized from ether as a white solid (76 mg, 97% yield). ¹H NMR (400 MHz, Methanol-d₄) δ 6.77 (s, 1H), 5.51 (q, J=3.6 Hz, 1H), 4.13 (ddd, J=12.7, 7.0, 4.5 Hz, 1H), 3.96 (ddd, J=12.7, 4.4, 3.0 Hz, 1H), 3.66 (s, 3H), 3.65-3.44 (m, 2H), 3.42-3.35 (m, 1H), 2.89 (d, J=3.1 Hz, 3H), 2.47-2.39 (m, 1H), 2.31-2.26 (m, 1H), 2.15 (s, 3H), 2.11-1.99 (m, 1H), 1.93-1.65 (m, 2H). LCMS m/z 328.92 [M+H]⁺.

### Step 3. Synthesis of N-[(2′S, 7R)-2-chloro-2′-methyl-1′-[[1-(2-methylsulfonylethyl)pyrazol-4-yl]methyl]spiro[4,5-dihydrothieno[2,3-c]pyran-7,4′-piperidine]-4-yl]-N-methyl-acetamide (857)

A mixture of N-[(2′S,7R)-2-chloro-2′-methyl-spiro[4,5-dihydrothieno[2,3-c]pyran-7,4′-piperidine]-4-yl]-N-methyl-acetamide C63 (20 mg, 0.054 mmol), 1-(2-methylsulfonylethyl)pyrazole-4-carbaldehyde (30 mg, 0.15 mmol), solid-supported NaBH₃CN (0.15 mmol) and AcOH (4 μL, 0.07 mmol) in DCM (1 mL) was stirred at 95° C. in a microwave reactor. After 3 h, the reaction was cooled and filtered. The resin was rinsed with methanol and the combined organic layers were concentrated in vacuo. Purification by silica gel chromatography (0 to 12% MeOH in DCM) yielded the product as a clear oil (10 mg, 35% yield). ¹H NMR (400 MHz, CDCl₃) δ 7.51 (s, 1H), 7.47 (s, 1H), 6.60 (dd, J=13.6, 2.3 Hz, 1H), 5.51 (ddd, J=11.2, 4.2, 2.0 Hz, 1H), 4.67-4.56 (m, 2H), 4.00 (ddd, J=12.7, 6.8, 4.2 Hz, 1H), 3.92-3.77 (m, 2H), 3.73-3.62 (m, 2H), 3.58 (d, J=14.4 Hz, 1H), 2.91-2.63 (m, 4H), 2.48 (d, J=3.4 Hz, 5H), 2.18-2.12 (m, 3H), 2.07-1.81 (m, 2H), 1.84-1.44 (m, 2H), 1.22 (t, J=6.3 Hz, 3H). LCMS m/z 514.94 [M+H]⁺.

### Compounds 858 and 859

### (2′S, 7R)-2-chloro-2′,3-dimethyl-1′-[[1-(2-methylsulfonylethyl)pyrazol-4-yl]methyl]spiro[4,5-dihydrothieno[2,3-c]pyran-7,4′-piperidine]-4-ol (858) and (2′S, 7R)-2-chloro-2′,3-dimethyl-1′-[[1-(2-methylsulfonylethyl)pyrazol-4-yl]methyl]spiro[4,5-dihydrothieno[2,3-c]pyran-7,4′-piperidine]-4-ol (859)

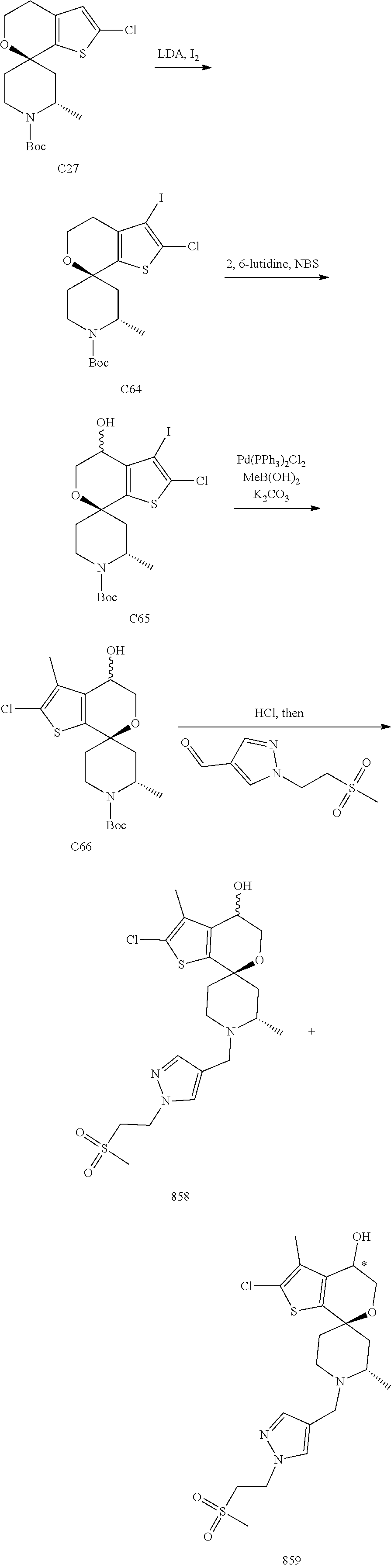

### Step 1. Synthesis of tert-butyl (2S,4R)-2′-chloro-3′-iodo-2-methyl-4′,5′-dihydrospiro[piperidine-4,7′-thieno[2,3-c]pyran]-1-carboxylate (C64)

To a solution of diisopropylamine (220 μL, 1.6 mmol) in THF (3 mL) under a nitrogen atmosphere was added n-BuLi (660 μL of 2.2 M, 1.5 mmol) dropwise at 0° C. The mixture was stirred for 30 min and transferred to a solution of tert-butyl (2′S)-2-chloro-2′-methyl-spiro[4,5-dihydrothieno[2,3-c]pyran-7,4′-piperidine]-1′-carboxylate C27 (500 mg, 1.397 mmol) in THF (3 mL) at −78° C. Then, iodine (500 mg, 2.0 mmol) in THF (0.5 mL) was added, and the mixture was stirred for 5 min and quenched with aqueous ammonium chloride solution. The mixture was warmed to room temperature and diluted with ethyl acetate. The organic layer was separated and concentrated in vacuo. The residue was purified by silica gel chromatography (Gradient: 0-15% EtOAc in heptane) to afford the product. tert-Butyl (2′S)-2-chloro-3-iodo-2′-methyl-spiro[4,5-dihydrothieno[2,3-c]pyran-7,4′-piperidine]-1′-carboxylate (380 mg, 56%). ¹H NMR (300 MHz, Chloroform-d) δ 4.00 (ddd, J=11.4, 6.6, 5.1 Hz, 1H), 3.91 (td, J=5.4, 1.1 Hz, 2H), 3.73 (ddd, J=14.0, 6.0, 4.9 Hz, 1H), 3.32 (ddd, J=14.0, 8.7, 5.4 Hz, 1H), 2.49 (td, J=5.5, 3.3 Hz, 2H), 2.24-2.01 (m, 2H), 1.82-1.71 (m, 1H), 1.66 (dd, J=14.1, 10.9 Hz, 1H), 1.49 (s, 9H), 1.30-1.23 (m, 3H). LCMS m/z 484.07 [M+H]⁺.

### Step 2. Synthesis of tert-butyl (2S,4R)-2′-chloro-4′-hydroxy-3′-iodo-2-methyl-4′,5′-dihydrospiro[piperidine-4,7′-thieno[2,3-c]pyran]-1-carboxylate (C65)

To a solution of tert-butyl (2′S)-2-chloro-3-iodo-2′-methyl-spiro[4,5-dihydrothieno[2,3-c]pyran-7,4′-piperidine]-1′-carboxylate C64 (380 mg, 0.79 mmol) in chlorobenzene (10 mL) was added 2,6-lutidine (220 μL, 1.9 mmol). The mixture was sparged with nitrogen, and NBS (220 mg, 1.2 mmol) was added. The mixture was irradiated under blue LED for 40 min. Next, the mixture was filtered, and the filtrate was concentrated in vacuo. The residue was dissolved in THF (5 mL), and saturated sodium bicarbonate solution was added. The mixture was heated to 70° C. for 60 h and diluted subsequently with ethyl acetate. The aqueous layer was extracted three times and the combined organic layer was dried over sodium sulfate, filtered, and concentrated in vacuo. The residue purified by silica gel chromatography (Gradient: 0-35% EtOAc in heptane) to afford the product. tert-Butyl (2′S)-2-chloro-4-hydroxy-3-iodo-2′-methyl-spiro[4,5-dihydrothieno[2,3-c]pyran-7,4′-piperidine]-1′-carboxylate (90 mg, 22%). ¹H NMR (300 MHz, Chloroform-d) δ 4.32 (s, 1H), 4.20-3.86 (m, 3H), 3.76 (tdd, J=11.0, 8.7, 5.1 Hz, 1H), 3.45-3.21 (m, 1H), 2.32-2.11 (m, 2H), 2.10-1.86 (m, 1H), 1.79-1.52 (m, 2H), 1.49 (s, 9H), 1.29-1.24 (m, 3H). LCMS m/z 499.88 [M+H]⁺.

### Step 3. Synthesis of tert-butyl (2S,4R)-2′-chloro-4′-hydroxy-2,3′-dimethyl-4′,5′-dihydrospiro[piperidine-4,7′-thieno[2,3-c]pyran]-1-carboxylate (C66)

To a solution of tert-butyl (2′S,7R)-2-chloro-4-hydroxy-3-iodo-2′-methyl-spiro[4,5-dihydrothieno[2,3-c]pyran-7,4′-piperidine]-1′-carboxylate C65 (171 mg, 0.342 mmol) in DME (4 mL) was added water (855 μL), CH₃B(OH)₂ (123 mg, 2.06 mmol), K₂CO₃ (95 mg, 0.69 mmol), and bis(triphenylphosphine)palladium(II) dichloride (24 mg, 0.034 mmol) at room temperature. The mixture was heated at 60° C. for 36 h. The mixture was partitioned between water and DCM, and the organic layer was separated and concentrated in vacuo. Purification by silica gel chromatography (Gradient: 0-60% EtOAc in heptane) yielded the product. tert-Butyl (2′S,7R)-2-chloro-4-hydroxy-2′,3-dimethyl-spiro[4,5-dihydrothieno[2,3-c]pyran-7,4′-piperidine]-1′-carboxylate (90 mg, 61%). LCMS m/z 384.75 [M+H]⁺.

### Step 4. Synthesis of (2S,4R)-2′-chloro-2,3′-dimethyl-1-((1-(2-(methylsulfonyl)ethyl)-1H-pyrazol-4-yl)methyl)-4′,5′-dihydrospiro[piperidine-4,7′-thieno[2,3-c]pyran]-4′-ol (858) and (2′S, 7R)-2-chloro-2′,3-dimethyl-1′-[[1-(2-methylsulfonylethyl)pyrazol-4-yl]methyl]spiro[4,5-dihydrothieno[2,3-c]pyran-7,4′-piperidine]-4-ol (859)

To a solution of tert-butyl (2′S,7R)-2-chloro-4-hydroxy-2′,3-dimethyl-spiro[4,5-dihydrothieno[2,3-c]pyran-7,4′-piperidine]-1′-carboxylate C66 (90 mg, 0.23 mmol) in 1,4-dioxane (855 μL) was added HCl (855 μL of 4 M, 3.420 mmol) in 1,4-dioxane. The mixture was stirred for 30 min and concentrated in vacuo. To the residue was added 1-(2-methylsulfonylethyl)pyrazole-4-carbaldehyde (84 mg, 0.42 mmol), acetic acid (60 mg, 1 mmol), dichloromethane (2 mL), and polymer-supported cyanoborohydride (310 mg of 2 mmol/g, 0.6200 mmol). The mixture was heated to 95° C. for 120 min. Next, MeOH was added, and the mixture was stirred for 10 min, filtered, and concentrated in vacuo. Purification by reversed-phase HPLC (Method: C18 Waters Sunfire column (30×150 mm, 5 micron). Gradient: MeCN in H₂O with 0.1% trifluoroacetic acid) yielded the products. (2′S,7R)-2-Chloro-2′,3-dimethyl-1′-[[1-(2-methylsulfonylethyl)pyrazol-4-yl]methyl]spiro[4,5-dihydrothieno[2,3-c]pyran-7,4′-piperidine]-4-ol (Trifluoroacetate salt) (31 mg, 25%) as a mixture of isomers.

¹H NMR (400 MHz, Methanol-d₄) δ 8.00 (dd, J=1.7, 0.8 Hz, 1H), 7.73 (d, J=0.8 Hz, 1H), 4.76-4.64 (m, 2H), 4.53 (dd, J=14.1, 1.8 Hz, 1H), 4.38 (dt, J=4.1, 2.1 Hz, 1H), 4.28 (d, J=14.1 Hz, 1H), 4.07-3.83 (m, 2H), 3.72 (t, J=6.2 Hz, 2H), 3.65-3.33 (m, 2H), 3.22 (td, J=13.0, 2.9 Hz, 1H), 2.89-2.82 (m, 3H), 2.57-1.72 (m, 7H), 1.53 (dd, J=6.5, 5.1 Hz, 3H). LCMS m/z 474.3 [M+H]⁺; and

(2′S,7R)-2-chloro-2′,3-dimethyl-1′-[[1-(2-methylsulfonylethyl)pyrazol-4-yl]methyl]spiro[4,5-dihydrothieno[2,3-c]pyran-7,4′-piperidine]-4-ol (Trifluoroacetate salt) (6 mg, 10%) as a single enantiomer. ¹H NMR (400 MHz, Methanol-d₄) δ 8.00 (d, J=0.8 Hz, 1H), 7.72 (d, J=0.9 Hz, 1H), 4.76-4.61 (m, 2H), 4.53 (d, J=14.1 Hz, 1H), 4.39 (t, J=2.1 Hz, 1H), 4.29 (d, J=14.1 Hz, 1H), 4.03-3.83 (m, 2H), 3.72 (t, J=6.2 Hz, 2H), 3.53-3.32 (m, 3H), 2.87 (d, J=0.6 Hz, 3H), 2.50 (dt, J=15.2, 3.0 Hz, 1H), 2.35-1.92 (m, 5H), 1.77 (dd, J=15.3, 12.1 Hz, 1H), 1.52 (d, J=6.5 Hz, 3H).

### Compounds 860 and 861

### (2′S, 7R)-2,3-dichloro-2′-methyl-1′-[[1-(2-methylsulfonylethyl)pyrazol-4-yl]methyl]spiro[4,5-dihydrothieno[2,3-c]pyran-7,4′-piperidine]-4-ol (860) and (2S,4R)-2′,3′-dichloro-2-methyl-1-((1-(2-(methylsulfonyl)ethyl)-1H-pyrazol-4-yl)methyl)-4′l3-spiro[piperidine-4,7′-thieno[3,2-d]pyran]-4′(5′H)-ol (861)

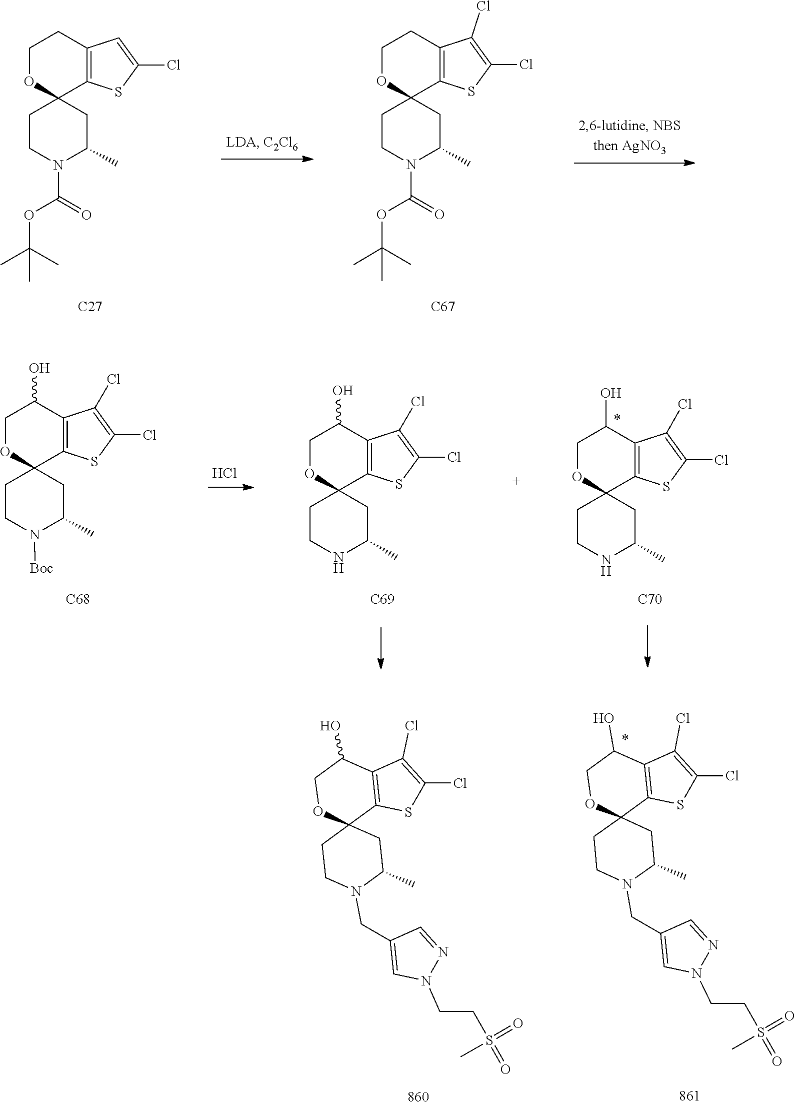

### Step 1. Synthesis of tert-butyl (2S,4R)-2′,3′-dichloro-2-methyl-4′,5′-dihydrospiro[piperidine-4,7′-thieno[2,3-c]pyran]-1-carboxylate (C67)

To a solution of diisopropylamine (176 μL, 1.26 mmol) in THF (2.4 mL) under a nitrogen atmosphere at 0° C. was added sec-BuLi (840 μL of 1.3 M, 1.1 mmol) dropwise. The mixture was stirred for 30 min and transferred to a solution of tert-butyl (2′S,7R)-2-chloro-2′-methyl-spiro[4,5-dihydrothieno[2,3-c]pyran-7,4′-piperidine]-1′-carboxylate C27 (400 mg, 1.1 mmol) in THF (2.4 mL) at −78° C. The mixture was stirred for 2 min, and hexachloroethane (370 mg, 1.6 mmol) in THF (0.5 mL) was added. The mixture was stirred for 5 min and quenched with aqueous ammonium chloride solution. The mixture was warmed to room temperature and diluted with DCM. The organic layer was separated and concentrated in vacuo. The residue was purified by silica gel chromatography (Gradient: 0-20% EtOAc in heptane) to afford the product. tert-Butyl (2′S,7R)-2,3-dichloro-2′-methyl-spiro[4,5-dihydrothieno[2,3-c]pyran-7,4′-piperidine]-1′-carboxylate (165 mg, 34%). LCMS m/z 392.4 [M+H]⁺.

### Step 2. Synthesis of tert-butyl (2S,4R)-2′,3′-dichloro-4′-hydroxy-2-methyl-4′,5′-dihydrospiro[piperidine-4,7′-thieno[2,3-c]pyran]-1-carboxylate (C68)

To a solution of tert-butyl (2′S)-2,3-dichloro-2′-methyl-spiro[4,5-dihydrothieno[2,3-c]pyran-7,4′-piperidine]-1′-carboxylate C67 (480 mg, 1.0 mmol) in chlorobenzene (12.6 mL) was added 2,6-lutidine (342 μL, 2.95 mmol). The mixture was sparged with nitrogen for 15 min, and NBS (291 mg, 1.64 mmol) was added. The mixture was irradiated with blue LED for 40 min and quenched with sodium bisulfate. The mixture was extracted with DCM, and the organic phase was washed with brine and concentrated in vacuo. The residue was dissolved in acetone (6.24 mL) and water (6.24 mL), and silver nitrate (226 mg, 1.33 mmol) was added. After 1 h, the mixture was concentrated in vacuo and partitioned between DCM and water. The organic layer was separated and concentrated in vacuo. Purification by silica gel chromatography (Gradient: 0-60% EtOAc in heptane) yielded the product. tert-Butyl (2S,4R)-2′,3′-dichloro-4′-hydroxy-2-methyl-4′,5′-dihydrospiro[piperidine-4,7′-thieno[2,3-c]pyran]-1-carboxylate (90 mg, 21%) LCMS m/z 407.75 [M+H]⁺.

### Step 3. Synthesis of (2S,4R)-2′,3′-dichloro-2-methyl-4′,5′-dihydrospiro[piperidine-4,7′-thieno[2,3-c]pyran]-4′-ol (C69)

To a solution of tert-butyl (2′S)-2,3-dichloro-4-hydroxy-2′-methyl-spiro[4,5-dihydrothieno[2,3-c]pyran-7,4′-piperidine]-1′-carboxylate C68 (90 mg, 21%) in 1,4-dioxane (2.4 mL) was added HCl (917 μL of 4 M, 3.668 mmol). The mixture was stirred for 5 h, concentrated in vacuo, and purified by reversed-phase chromatography (Column: C18. Gradient: 0-100% MeCN in water with 0.1% TFA) to afford the product which was subsequently partitioned between DCM and 1 M NaOH solution. The organic layer was collected and concentrated in vacuo to give the product. (2′S,7S)-2,3-Dichloro-2′-methyl-spiro[4,5-dihydrothieno[2,3-c]pyran-7,4′-piperidine]-4-ol (35.1 mg, 50%) as a mixture of isomers. LCMS m/z 329.74 [M+H]⁺; (2′S,7R)-2,3-dichloro-2′-methyl-spiro[4,5-dihydrothieno[2,3-c]pyran-7,4′-piperidine]-4-ol (8 mg, 25%) as a single stereoisomer. ¹H NMR (400 MHz, Methanol-d₄) δ 4.40 (dd, J=2.3, 1.6 Hz, 1H), 4.00 (dd, J=12.6, 2.3 Hz, 1H), 3.92 (dd, J=12.6, 1.7 Hz, 1H), 3.13 (td, J=12.7, 2.9 Hz, 1H), 3.08-2.87 (m, 2H), 2.21 (dt, J=14.2, 2.7 Hz, 1H), 1.99 (dq, J=13.7, 2.6 Hz, 1H), 1.78 (td, J=13.1, 4.8 Hz, 1H), 1.20 (dd, J=14.2, 11.5 Hz, 1H), 1.09 (d, J=6.5 Hz, 3H). LCMS m/z 307.94 [M+H]⁺.

### Step 4a. Synthesis of (2′S, 7R)-2,3-dichloro-2′-methyl-1′-[[1-(2-methylsulfonylethyl)pyrazol-4-yl]methyl]spiro[4,5-dihydrothieno[2,3-c]pyran-7,4′-piperidine]-4-ol (860)

To a solution of (2S,4R)-2′,3′-dichloro-2-methyl-4′,5′-dihydrospiro[piperidine-4,7′-thieno[2,3-c]pyran]-4′-ol C69 (8 mg, 0.03 mmol), 1-(2-methylsulfonylethyl)pyrazole-4-carbaldehyde (10.5 mg, 0.0519 mmol), acetic acid (7.5 mg, 0.12 mmol) in dichloromethane (2 mL) was added polymer-supported cyanoborohydride (40 mg of 2 mmol/g, 0.080 mmol). The mixture was heated to 95° C. in a microwave reactor for 120 min. Then, MeOH was added, and the mixture was stirred for 10 min, filtered, and concentrated in vacuo. Purification by reversed-phase HPLC (Method: C18 Waters Sunfire column (30×150 mm, 5 micron). Gradient: MeCN in H₂O with 0.1% trifluoroacetic acid) afforded the product which was partitioned between DCM and pH 10 buffer. The organic layer was separated and concentrated in vacuo to afford the product. (2′S,7R)-2,3-dichloro-2′-methyl-1′-[[1-(2-methylsulfonylethyl)pyrazol-4-yl]methyl]spiro[4,5-dihydrothieno[2,3-c]pyran-7,4′-piperidine]-4-ol (8.3 mg, 65%). ¹H NMR (400 MHz, Methanol-d₄) δ 8.00 (d, J=2.5 Hz, 1H), 7.73 (s, 1H), 4.70 (dd, J=7.0, 5.2 Hz, 2H), 4.61-4.46 (m, 1H), 4.42 (dt, J=3.3, 1.8 Hz, 1H), 4.29 (d, J=14.1 Hz, 1H), 4.06-3.87 (m, 2H), 3.72 (t, J=6.2 Hz, 2H), 3.54-3.33 (m, 2H), 2.87 (dd, J=5.1, 2.4 Hz, 3H), 2.64-1.71 (m, 4H), 1.54 (t, J=6.3 Hz, 3H). LCMS m/z 494.1 [M+H]⁺.

### Step 4b. Synthesis of (2S,4R)-2′,3′-dichloro-2-methyl-1-((1-(2-(methylsulfonyl)ethyl)-1H-pyrazol-4-yl)methyl)-4′l3-spiro[piperidine-4,7′-thieno[3,2-d]pyran]-4′(5′H)-ol (861)

To a solution of (2′S,7R)-2,3-dichloro-2′-methyl-spiro[4,5-dihydrothieno[2,3-c]pyran-7,4′-piperidine]-4-ol 6C69 (11 mg, 0.036 mmol), 1-(2-methylsulfonylethyl)pyrazole-4-carbaldehyde (14 mg, 0.069 mmol), and acetic acid (10 mg, 0.2 mmol) in dichloromethane (2.75 mL) was added polymer-supported cyanoborohydride (55 mg of 2 mmol/g, 0.11 mmol). The mixture was heated to 95° C. in a microwave reactor for 120 min. Then, MeOH was added, and the mixture was stirred for 10 min, filtered, and concentrated in vacuo. Purification by reversed-phase HPLC (Method: C18 Waters Sunfire column (30×150 mm, 5 micron). Gradient: MeCN in H₂O with 0.1% formic acid) yielded product. (2′S,7R)-2,3-dichloro-2′-methyl-1′-[[1-(2-methylsulfonylethyl)pyrazol-4-yl]methyl]spiro[4,5-dihydrothieno[2,3-c]pyran-7,4′-piperidine]-4-ol (Formic acid salt) (7.8 mg, 40%). ¹H NMR (400 MHz, Methanol-d₄) δ 7.98 (d, J=0.7 Hz, 1H), 7.72 (d, J=0.7 Hz, 1H), 4.73-4.63 (m, 2H), 4.53-4.20 (m, 3H), 3.93 (d, J=1.8 Hz, 2H), 3.78-3.66 (m, 2H), 3.57 (ddd, J=12.0, 6.4, 3.0 Hz, 1H), 3.19 (td, J=13.0, 2.9 Hz, 1H), 2.85 (s, 3H), 2.41 (dt, J=15.2, 2.9 Hz, 1H), 2.28 (dt, J=14.5, 3.1 Hz, 1H), 2.06 (dd, J=14.6, 12.0 Hz, 1H), 1.88 (ddd, J=15.1, 13.2, 4.4 Hz, 1H), 1.53 (d, J=6.5 Hz, 3H). LCMS m/z 494.39 [M+H]⁺.

### Compound 862

### (2S,4R)-2′-chloro-4′,4′-difluoro-2-methyl-1-((1-(2-(methylsulfonyl)ethyl)-1H-1,2,3-triazol-4-yl)methyl)-4′,5′-dihydrospiro[piperidine-4,7′-thieno[2,3-c]pyran](JMA5)

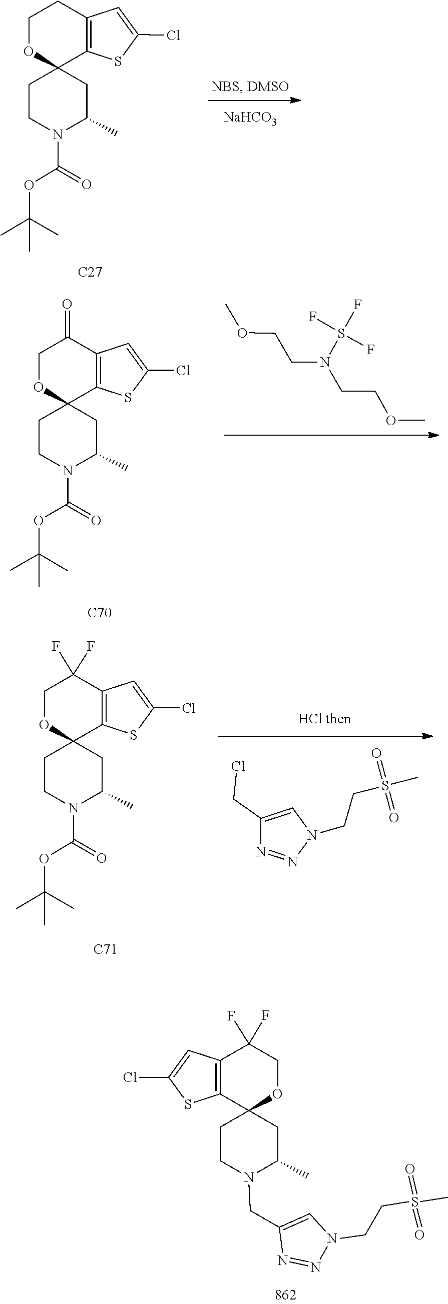

### Step 1. Synthesis of tert-butyl (2S,4R)-2′-chloro-2-methyl-4′-oxo-4′,5′-dihydrospiro[piperidine-4,7′-thieno[2,3-c]pyran]-1-carboxylate (C70)

A solution of tert-butyl (2′S,7R)-2-chloro-2′-methyl-spiro[4,5-dihydrothieno[2,3-c]pyran-7,4′-piperidine]-1′-carboxylate C27 (900 mg, 1.8 mmol) and NBS (400 mg, 2.2 mmol) in chlorobenzene (12 mL) was irradiated under a 100 W light bulb overnight. Additional NBS (83 mg, 0.47 mmol) was added, and the mixture was stirred for additional 5 h. Next, sodium bisulfate was added, and the mixture was extracted with DCM, filtered through a pad of silica, and concentrated in vacuo. The residue was dissolved in DMSO (36 mL), and sodium bicarbonate (236 mg, 2.81 mmol) was added. The mixture was heated to 50° C. overnight. Then, water was added and the mixture was extracted with DCM (×3), washed with brine, and concentrated in vacuo. The residue was purified by silica gel chromatography (Gradient: 0-20% EtOAc in heptane) to afford the product. tert-Butyl (2S,4R)-2′-chloro-2-methyl-4′-oxo-spiro[piperidine-4,7′-thieno[2,3-c]pyran]-1-carboxylate (80 mg, 8%). LCMS m/z 372.1 [M+H]⁺.

### Step 2. Synthesis of tert-butyl (2S,4R)-2′-chloro-4′,4′-difluoro-2-methyl-4′,5′-dihydrospiro[piperidine-4,7′-thieno[2,3-c]pyran]-1-carboxylate (C71)

To a solution of tert-butyl (2S,4R)-2′-chloro-2-methyl-4′-oxo-spiro[piperidine-4,7′-thieno[2,3-c]pyran]-1-carboxylate C70 (80 mg, 0.2 mmol) in toluene (2 mL) at 65° C. was added Deoxo-fluor (119 mg, 0.538 mmol) dropwise. The mixture was allowed to stir overnight then additional Deoxo-fluor (238 mg, 1.08 mmol) was added. The mixture was heated to 76° C. and stirred for 24 h. Then, the mixture was diluted with DCM and washed with NaHCO₃ and brine. The organic layer was concentrated in vacuo, and the residue was purified by silica gel chromatography (Gradient: 0-25% EtOAc in heptane) to afford the product. tert-Butyl (2′S,7R)-2-chloro-4,4-difluoro-2′-methyl-spiro[5H-thieno[2,3-c]pyran-7,4′-piperidine]-1′-carboxylate (8 mg, 9%). LCMS m/z 391.63 [M+H]⁺.

### Step 3. Synthesis of (2S,4R)-2′-chloro-4′,4′-difluoro-2-methyl-1-((1-(2-(methylsulfonyl)ethyl)-1H-1,2,3-triazol-4-yl)methyl)-4′,5′-dihydrospiro[piperidine-4,7′-thieno[2,3-c]pyran] (862)

To a solution of tert-butyl (2S,4R)-2′-chloro-4′,4′-difluoro-2-methyl-4′,5′-dihydrospiro[piperidine-4,7′-thieno[2,3-c]pyran]-1-carboxylate C71 (8 mg, 0.02 mmol) in 1,4-dioxane (0.3 mL) was added HCl (0.051 mL, 4 M in 1,4-dioxane). The mixture was stirred overnight and concentrated in vacuo. To the residue was added 4-(chloromethyl)-1-(2-(methylsulfonyl)ethyl)-1H-1,2,3-triazole (9 mg, 0.04 mmol), DCE (0.2 mL), and DIPEA (7.8 mg, 0.060 mmol). The mixture was heated to 60° C. overnight. Then, the mixture was partitioned between DCM and 1 M NaOH, and the organic layer was separated and concentrated in vacuo. Purification by reversed-phase chromatography (Column: C18. Gradient: 0-100% MeCN in water with 0.1% TFA) afforded the product. (2S,4R)-2′-chloro-4′,4′-difluoro-2-methyl-1-((1-(2-(methylsulfonyl)ethyl)-1H-1,2,3-triazol-4-yl)methyl)-4′,5′-dihydrospiro[piperidine-4,7′-thieno[2,3-c]pyran] (2.4 mg, 20%). ¹H NMR (300 MHz, Methanol-d₄) δ 8.33 (s, 1H), 7.14 (s, 1H), 5.06-4.95 (m, 2H), 4.77-4.54 (m, 2H), 4.10 (t, J=10.4 Hz, 2H), 3.90-3.78 (m, 2H), 3.66-3.34 (m, 3H), 3.01 (s, 3H), 2.56-2.35 (m, 2H), 2.17-1.90 (m, 2H), 1.58 (d, J=6.5 Hz, 3H). LCMS m/z 480.99 [M+H]⁺.

### Compound 863

### (2S,4R)-2′-chloro-4′-fluoro-2-methyl-1-((1-(2-(methylsulfonyl)ethyl)-1H-1,2,3-triazol-4-yl)methyl)-4′,5′-dihydrospiro[piperidine-4,7′-thieno[2,3-c]pyran] (863)

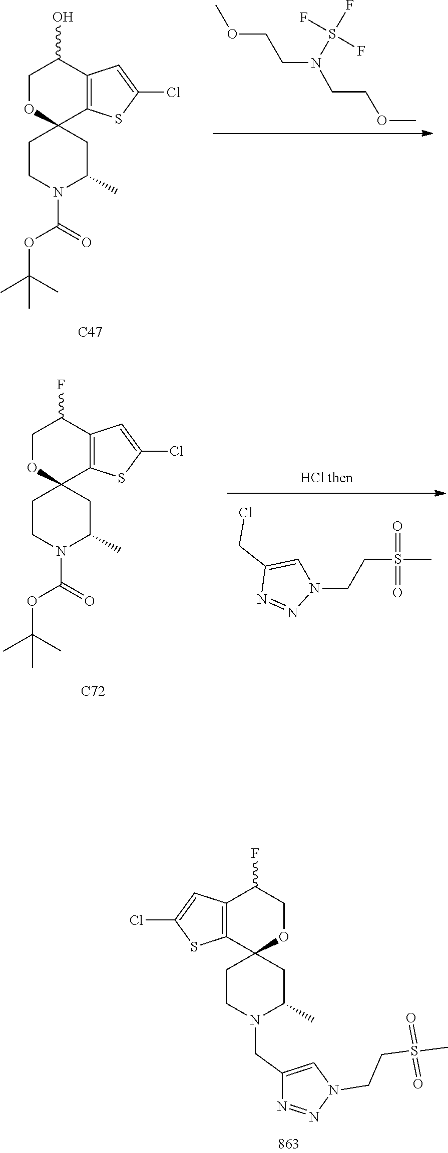

### Step 1. Synthesis of tert-butyl (2S,4R)-2′-chloro-4′-fluoro-2-methyl-4′,5′-dihydrospiro[piperidine-4,7′-thieno[2,3-c]pyran]-1-carboxylate (C72)

To a solution of tert-butyl (2′S,7R)-2-chloro-4-hydroxy-2′-methyl-spiro[4,5-dihydrothieno[2,3-c]pyran-7,4′-piperidine]-1′-carboxylate C47 (82 mg, 0.22 mmol) in DCM (7.2 mL) at −78° C. was added 2-methoxy-N-(2-methoxyethyl)-N-(trifluoro-Î»4-sulfanyl)ethanamine (126 mg, 0.57 mmol). The mixture was stirred for 1 h and quenched with saturated ammonium chloride solution. The organic layer was separated and concentrated in vacuo to afford the product. tert-Butyl (2′S,7R)-2-chloro-4-fluoro-2′-methyl-spiro[4,5-dihydrothieno[2,3-c]pyran-7,4′-piperidine]-1′-carboxylate (80 mg, 72%). LCMS m/z 376.29 [M+H]⁺.

### Step 2. Synthesis of (2S,4R)-2′-chloro-4′-fluoro-2-methyl-1-((1-(2-(methylsulfonyl)ethyl)-1H-1, 2, 3-triazol-4-yl)methyl)-4′,5′-dihydrospiro[piperidine-4,7′-thieno[2,3-c]pyran] (863)

To a solution of rac-tert-butyl (2′R)-2-chloro-4-fluoro-2′-methyl-5,6-dihydro-4H-spiro[benzo[b]thiophene-7,4′-piperidine]-1′-carboxylate C72 (90 mg, 0.24 mmol) in 1,4-dioxane (3.6 mL) was added HCl (0.6 mL, 4 M in 1,4-dioxane). The mixture was stirred overnight and concentrated in vacuo. To the residue was added 4-(chloromethyl)-1-(2-methylsulfonylethyl)triazole (62 mg, 0.028 mmol), DCE (2.6 mL), and DIPEA (92 mg, 0.71 mmol). The mixture was heated to 60° C. overnight. Then, the mixture was partitioned between DCM and 1 M NaOH, and the organic layer was separated and concentrated in vacuo. Purification by reversed-phase chromatography (Column: C18. Gradient: 0-100% MeCN in water with 0.1% TFA) afforded the product. (2S,4R)-2′-chloro-4′-fluoro-2-methyl-1-((1-(2-(methylsulfonyl)ethyl)-1H-1,2,3-triazol-4-yl)methyl)-4′,5′-dihydrospiro[piperidine-4,7′-thieno[2,3-c]pyran] (46 mg, 30%). ¹H NMR (300 MHz, Methanol-d₄) δ 8.34 (d, J=2.7 Hz, 1H), 7.00 (d, J=0.7 Hz, 1H), 5.23 (ddt, J=51.1, 3.4, 2.0 Hz, 1H), 5.11-4.94 (m, 2H), 4.63 (q, J=14.4 Hz, 2H), 4.19-3.91 (m, 2H), 3.83 (t, J=6.3 Hz, 2H), 3.66-3.37 (m, 2H), 3.00 (d, J=0.6 Hz, 3H), 2.65-1.78 (m, 5H), 1.57 (dd, J=6.5, 5.4 Hz, 3H). LCMS m/z 463.11 [M+H]⁺.

### Preparation S64 and S65

### tert-butyl (2′S, 7R)-2′-methyl-2-(trifluoromethyl)spiro[4,5-dihydrothieno[2,3-c]pyran-7,4′-piperidine]-1′-carboxylate (S64) and (1r,3r)-3-[5,7-difluoro-2-(4-fluorophenyl)-1H-indol-3-yl]cyclobutanamine (S65)

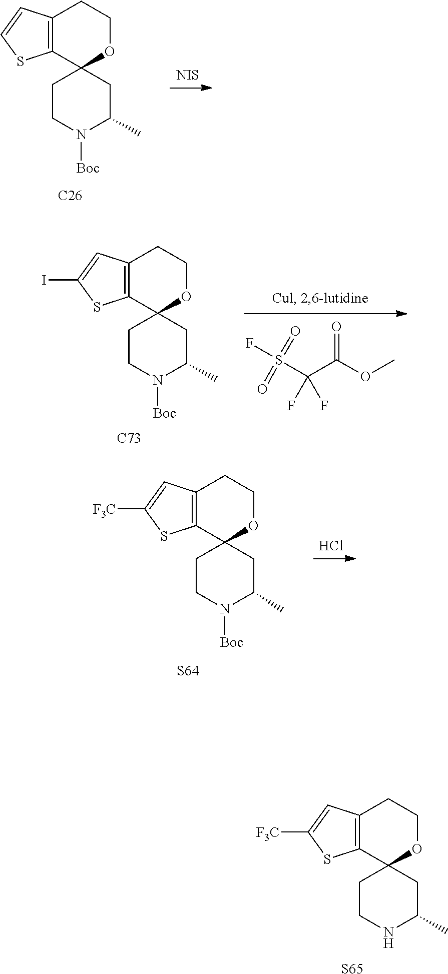

### Step 1. Synthesis of tert-butyl (2′S, 7R)-2-iodo-2′-methyl-spiro[4,5-dihydrothieno[2,3-c]pyran-7,4′-piperidine]-1′-carboxylate (C73)

In a 3-neck RBF equipped with a mechanical stirrer and a temperature probe, and a nitrogen inlet/outlet to a solution of tert-butyl (2′S,7R)-2′-methylspiro[4,5-dihydrothieno[2,3-c]pyran-7,4′-piperidine]-1′-carboxylate C26 (227 g, 701.8 mmol) in DCM (2.5 L) and AcOH (680 mL) was added NIS (206 g, 869.8 mmol) over 10 minutes. The resulting reaction mixture was stirred at rt for 30 h. The reaction mixture was basified with aqueous 2 N NaOH solution (˜8 L) until pH=11. The organic phase was separated, washed with 10% aqueous sodium bisulfite (1 L), water (600 mL), brine (2 L) and dried over MgSO₄, filtered and concentrated under reduced pressure. The crude residue was purified by Combiflash Torrent, 3 kg isco column linear gradient (0% to 40% ethyl acetate in heptane), fractions which contained desired product were collected, concentrated under reduced pressure to afford tert-butyl (2′S,7R)-2-iodo-2′-methyl-spiro[4,5-dihydrothieno[2,3-c]pyran-7,4′-piperidine]-1′-carboxylate (295 g, 85%) as a clear, light yellow viscous oil. 1H NMR (300 MHz, Chloroform-d) δ 6.89 (s, 1H), 3.98 (dt, J=11.5, 5.8 Hz, 1H), 3.83 (t, J=5.5 Hz, 2H), 3.71 (dt, J=14.0, 5.5 Hz, 1H), 3.32 (ddd, J=14.0, 8.7, 5.4 Hz, 1H), 2.71-2.53 (m, 2H), 2.20 (d, J=7.1 Hz, 2H), 1.86-1.61 (m, 2H), 1.48 (s, 9H), 1.25 (d, J=6.6 Hz, 3H).

### Step 2. Synthesis of tert-butyl (2′S, 7R)-2′-methyl-2-(trifluoromethyl)spiro[4,5-dihydrothieno[2,3-c]pyran-7,4′-piperidine]-1′-carboxylate (S64)

In a 4-neck RBF equipped with a mechanical stirrer, a temperature probe, a heating jacket, N₂ sweep to 6 N aq NaOH scrubber, to a solution/suspension of tert-butyl (2′S,7R)-2-iodo-2′-methyl-spiro[4,5-dihydrothieno[2,3-c]pyran-7,4′-piperidine]-1′-carboxylate C73 (295 g, 649.9 mmol), 2,6-lutidine (18 mL, 155.4 mmol) and CuI (127 g, 666.8 mmol) in DMF (4 L) preheated to 85° C., was added methyl 2,2-difluoro-2-fluorosulfonyl-acetate (165 mL, 1.296 mol) dropwise over the course of 30 minutes. After 2 h, HPLC-analysis revealed ˜50% conversion, added another 1.3 equiv of methyl 2,2-difluoro-2-fluorosulfonyl-acetate (110 mL, 864.0 mmol) over 20 minutes at 85° C. And the reaction mixture was stirred at this temperature for 2 h, at which time HPLC-analysis revealed ˜80% conversion. Added another 0.8 equiv of methyl 2,2-difluoro-2-fluorosulfonyl-acetate (67 mL, 526.3 mmol) over 20 minutes at 85° C. And the reaction mixture was stirred at this temperature for 12 h, at which time HPLC-analysis revealed ˜98% conversion to the desired product. The reaction mixture was cooled to ambient temperature and treated with MTBE (4 L), observed red color precipitate which was filtered through Celite®-bed, and the solid was washed with MTBE (3×1 L). Combined filtrates were washed with 1:1 water:ammonium hydroxide (ice cold, 4 L), organic phase was separated. The organic phase was washed again with 1:1 water:ammonium hydroxide (ice cold, 2 L), brine (1 L), 1 M aqueous HCl (1 L), brine (1 L), dried over MgSO₄, filtered and concentrated. The crude residue was purified by Combiflash Torrent, 1.5 kg isco column linear gradient (0% to 50% ethyl acetate in heptane), fractions which contained desired product were collected, concentrated under reduced pressure to afford tert-butyl (2′S,7R)-2′-methyl-2-(trifluoromethyl)spiro[4,5-dihydrothieno[2,3-c]pyran-7,4′-piperidine]-1′-carboxylate (230 g, 90%) as a clear, light yellow viscous oil. ¹H NMR (300 MHz, Chloroform-d) δ 7.09 (d, J=1.4 Hz, 1H), 4.01 (ddd, J=11.5, 6.8, 5.2 Hz, 1H), 3.94-3.80 (m, 2H), 3.80-3.64 (m, 1H), 3.35 (ddd, J=14.0, 8.7, 5.4 Hz, 1H), 2.66 (td, J=5.5, 3.3 Hz, 2H), 2.30-2.06 (m, 2H), 1.88-1.61 (m, 2H), 1.48 (s, 9H), 1.27 (d, J=6.5 Hz, 3H). ¹⁹F NMR (282 MHz, Chloroform-d) δ−55.35.

### Step 3. Synthesis of (2′S, 7R)-2′-methyl-2-(trifluoromethyl)spiro[4,5-dihydrothieno[2,3-c]pyran-7,4′-piperidine](S65)

Tert-butyl (2′S,7R)-2′-methyl-2-(trifluoromethyl)spiro[4,5-dihydrothieno[2,3-c]pyran-7,4′-piperidine]-1′-carboxylate S64 (466 mg, 1.190 mmol) in MeOH (5 ml) and HCl (3 mL of 4 M, 12.00 mmol) was stirred at 50° C. for 40 min. The reaction mixture was evaporated to provide (2′S,7R)-2′-methyl-2-(trifluoromethyl)spiro[4,5-dihydrothieno[2,3-c]pyran-7,4′-piperidine](hydrochloride salt) (310 mg, 78%). LCMS m/z 292.01 [M+1]⁺.

### Preparation 67

### [(2′S, 7R)-2′-methyl-2-(trifluoromethyl)spiro[4,5-dihydrothieno[2,3-c]pyran-7,4′-piperidine]-3-yl]methanol (S67)

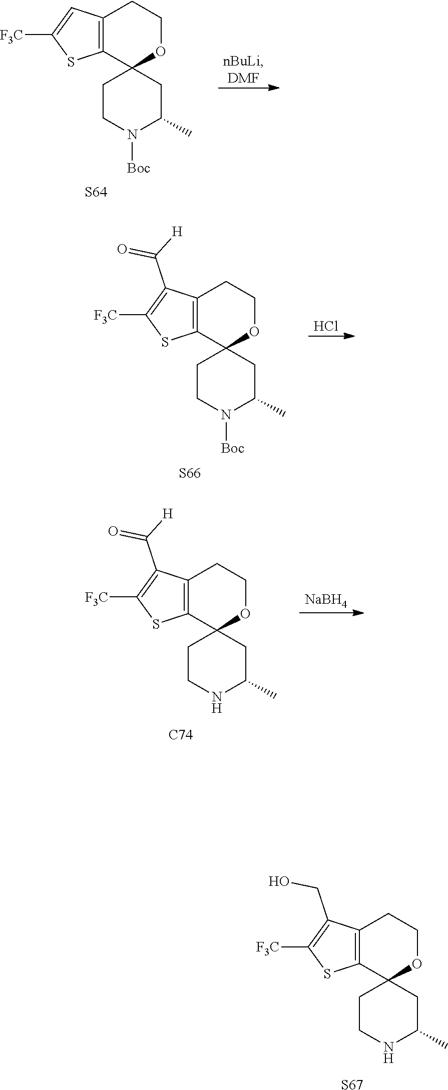

### Step 1. Synthesis of tert-butyl (2′S, 7R)-3-formyl-2′-methyl-2-(trifluoromethyl)spiro[4,5-dihydrothieno[2,3-c]pyran-7,4′-piperidine]-1′-carboxylate (S66)

To a solution of tert-butyl (2′S,7R)-2′-methyl-2-(trifluoromethyl)spiro[4,5-dihydrothieno[2,3-c]pyran-7,4′-piperidine]-1′-carboxylate S64 (230 g, 575.8 mmol) in THF (2.6 L) at −78° C. (dry ice/acetone bath) under N₂ was added n-BuLi (340 mL of 1.75 M, 595.0 mmol) over 30 minutes. After 5 min, an aliquot (0.3 mL) was quenched with CD₃OD (1 mL), solution was concentrated; 1H NMR shows virtually complete deuteration at the aromatic proton at 7.09 ppm. The reaction mixture was stirred at −78° C. for a total of 30 min following completion of addition, then DMF (270 mL, 3.487 mol) was added via addition funnel over the course of 6 min. Stirred at −78° C. then cooling bath was replaced with an ice-water bath. After 45 min in ice-water bath, reaction was quenched with water (500 mL). The reaction mixture was partitioned between MTBE (2 L) and water (1 L). The organic layer was isolated and washed successively with water (3×500 mL) then brine (1 L), dried using MgSO₄, filtered and concentrated. The residue was purified by silica gel chromatography (Gradient: 0-50% EtOAc in heptane), fractions which contained desired product were collected, concentrated under reduced pressure to afford tert-butyl (2′S,7R)-3-formyl-2′-methyl-2-(trifluoromethyl)spiro[4,5-dihydrothieno[2,3-c]pyran-7,4′-piperidine]-1′-carboxylate (215 g, 89%) as a clear, light yellow foam. ¹H NMR (300 MHz, Chloroform-d) δ 10.18 (d, J=1.4 Hz, 1H), 4.01 (ddd, J=11.5, 6.8, 5.2 Hz, 1H), 3.89 (td, J=5.6, 2.2 Hz, 2H), 3.75 (dt, J=14.0, 5.5 Hz, 1H), 3.38 (ddd, J=14.0, 8.6, 5.3 Hz, 1H), 3.04-2.87 (m, 2H), 2.24 (dddd, J=10.5, 8.3, 6.1, 3.0 Hz, 1H), 2.14 (ddd, J=14.1, 5.1, 1.9 Hz, 1H), 1.84-1.63 (m, 2H), 1.48 (s, 9H), 1.28 (d, J=6.6 Hz, 3H). ¹⁹F NMR (282 MHz, Chloroform-d) δ−50.49.

### Step 2. Synthesis of (2′S, 7R)-2′-methyl-2-(trifluoromethyl)spiro[4,5-dihydrothieno[2,3-c]pyran-7,4′-piperidine]-3-carbaldehyde (C74)

In a 3-neck RBF equipped with a mechanical stirrer, a temperature probe and an addition funnel, to a solution of tert-butyl (2′S,7R)-3-formyl-2′-methyl-2-(trifluoromethyl)spiro[4,5-dihydrothieno[2,3-c]pyran-7,4′-piperidine]-1′-carboxylate S66 (215 g, 512.6 mmol) in DCM (1.6 L) at 20° C. was added HCl (600 mL of 4 M, 2.400 mol) solution in dioxane dropwise over the course of 25 min. The reaction mixture was stirred for 3 h, and then cooled in ice-water bath to 0° C., then treated with aqueous 2 M NaOH (1.2 L, until pH=11.5) over the course of 20 min, slowly at first. The reaction mixture was stirred 5 min, and then sodium chloride (400 g) was added. Stirred for another 15 min, then layers were separated. Aqueous layer was re-extracted with DCM (2×200 mL). Combined organics were dried (MgSO₄) filtered and concentrated to afford (2′S,7R)-2′-methyl-2-(trifluoromethyl)spiro[4,5-dihydrothieno[2,3-c]pyran-7,4′-piperidine]-3-carbaldehyde (179 g, 100%) as a thick amber oil. ¹H NMR (300 MHz, Chloroform-d) δ 10.19 (t, J=1.3 Hz, 1H), 3.93 (td, J=5.7, 3.8 Hz, 2H), 3.21-3.03 (m, 2H), 3.03-2.87 (m, 3H), 2.06 (dd, J=13.9, 2.2 Hz, 2H), 1.79-1.63 (m, 1H), 1.39 (dd, J=13.4, 11.3 Hz, 1H), 1.08 (d, J=6.4 Hz, 3H). ¹⁹F NMR (282 MHz, Chloroform-d) δ −50.41. LCMS m/z 319.99 [M+1]⁺. This material was taken into the next step without further purification.

### Step 3. Synthesis of [(2′S, 7R)-2′-methyl-2-(trifluoromethyl)spiro[4,5-dihydrothieno[2,3-c]pyran-7,4′-piperidine]-3-yl]methanol (S67)

In a 3-neck RBF equipped with a mechanical stirrer, a temperature probe and an ice-water cooling bath, to a solution of (2′S,7R)-2′-methyl-2-(trifluoromethyl)spiro[4,5-dihydrothieno[2,3-c]pyran-7,4′-piperidine]-3-carbaldehyde C74 (179 g, 510.1 mmol) in a mixture of MeOH (1.5 L) and water (25 mL) at 0° C. (ice-water bath) was added NaBH₄ (13.5 g, 356.8 mmol) (granular, 10-40 mesh) in three portions, and gas evolution was observed. The resulting reaction mixture was stirred from 0° C. to rt over 2 h. The reaction mixture was concentrated in vacuo to remove most of methanol. The residue was dissolved in 1 M aqueous HCl (1.15 L), stirred for 15 min, then treated with DCM (1.0 L). The biphasic solution was stirred and slowly basified to ˜pH 10 with 2 M aqueous NaOH (600 mL). After stirring for 10 min, the layers were separated. Aqueous layer was extracted with DCM (2×400 mL). The combined organics were dried with MgSO₄, filtered and concentrated. The crude residue was treated with heptane (1.5 L), spun using rotavap (no vacuum) at 95° C. for 45 min, and not everything went into solution. The reaction mixture then stood at room temperature for 30 min, and cooled to 0° C. (on rotovap, no vacuum, ice-water bath) for 15 min. The resulting suspension was filtered, washed with heptane (2×200 mL). Dried under suction for 40 min and dried in vacuum oven at 50° C. for 1 h to afford [(2′S,7R)-2′-methyl-2-(trifluoromethyl)spiro[4,5-dihydrothieno[2,3-c]pyran-7,4′-piperidine]-3-yl]methanol (146 g, 89%) as a white crystalline solid. ¹H NMR (300 MHz, DMSO-d₆) δ 5.22 (t, J=5.3 Hz, 1H), 4.52-4.37 (m, 2H), 3.89 (td, J=5.7, 2.3 Hz, 2H), 2.96-2.79 (m, 2H), 2.76 (dd, J=4.9, 2.0 Hz, 1H), 2.66 (t, J=5.5 Hz, 2H), 1.91 (ddt, J=13.8, 8.0, 2.4 Hz, 3H), 1.54 (td, J=12.8, 4.8 Hz, 1H), 1.23 (dd, J=13.4, 11.2 Hz, 1H), 0.94 (d, J=6.3 Hz, 3H). ¹⁹F NMR (282 MHz, DMSO-d₆) δ−51.03. LCMS m/z 322.03 [M+1]⁺.

### Preparation S68 and S69

### 1-[(2′S, 7R)-2′-methyl-2-(trifluoromethyl)spiro[4,5-dihydrothieno[2,3-c]pyran-7,4′-piperidine]-3-yl]ethanol (S68 and S69)

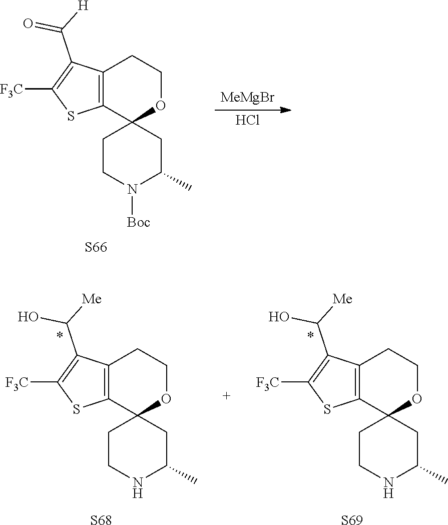

To a mixture of tert-butyl (2′S,7R)-3-formyl-2′-methyl-2-(trifluoromethyl)spiro[4,5-dihydrothieno[2,3-c]pyran-7,4′-piperidine]-1′-carboxylate S66 (500 mg, 1.192 mmol) in THF (10 mL) was added a THF solution of bromo(methyl)magnesium (500 μL of 3.4 M, 1.700 mmol). After 20 min, the mixture was diluted with sat. aq. ammonium chloride (1 mL) and ethyl ether (3 mL), and the organic layer was removed, concentrated, and minimally diluted in heptane for purification using silica gel chromatography (Gradient: 0-60% EtOAc in heptane), yielding 1-[(2′S,7R)-2′-methyl-2-(trifluoromethyl)spiro[4,5-dihydrothieno[2,3-c]pyran-7,4′-piperidine]-3-yl]ethanol (hydrochloride salt) (400 mg, 90%). The product was diluted with dioxane HCl (5 mL of 4 M, 20.00 mmol) and the mixture was stirred at rt. After 50 min, the mixture was concentrated in vacuo and the mixture was separated into constituent enantiomers by chiral SFC separation. Column: Daicel Chiralpak® AD-H, 20×250 mm; Mobile Phase: 20% isopropyl alcohol (containing 5 mM ammonia), 80% carbon dioxide. Flow: 75 mL/min. The two enantiomers were obtained:

1-[(2′S,7R)-2′-methyl-2-(trifluoromethyl)spiro[4,5-dihydrothieno[2,3-c]pyran-7,4′-piperidine]-3-yl]ethanol (152 mg, 75%). ¹H NMR (400 MHz, Chloroform-d) δ 5.33-5.23 (m, 1H), 4.02-3.83 (m, 2H), 3.46 (d, J=11.9 Hz, 1H), 3.32-3.18 (m, 2H), 3.05 (dt, J=16.3, 4.8 Hz, 1H), 2.82 (ddd, J=16.4, 7.1, 4.9 Hz, 1H), 2.23-2.09 (m, 3H), 1.87 (t, J=12.9 Hz, 1H), 1.53 (d, J=6.7 Hz, 3H), 1.38 (d, J=6.4 Hz, 3H). LCMS m/z 336.03 [M+1]⁺; and

1-[(2′S,7R)-2′-methyl-2-(trifluoromethyl)spiro[4,5-dihydrothieno[2,3-c]pyran-7,4′-piperidine]-3-yl]ethanol (152 mg, 75%) ¹H NMR (400 MHz, Chloroform-d) δ 5.27 (q, J=6.6 Hz, 1H), 3.95 (t, J=5.5 Hz, 2H), 3.15 (td, J=12.5, 2.6 Hz, 2H), 3.01 (tt, J=10.2, 5.1 Hz, 2H), 2.80 (dt, J=16.2, 5.5 Hz, 1H), 2.08 (ddt, J=14.1, 5.2, 2.6 Hz, 2H), 1.83-1.74 (m, 1H), 1.53 (d, J=6.7 Hz, 3H), 1.47-1.38 (m, 1H), 1.28 (s, 1H), 1.12 (d, J=6.4 Hz, 3H). LCMS m/z 336.03 [M+1]⁺.

### Preparation S70

### (2′S, 7R)-2′-methyl-2-(trifluoromethyl)spiro[4,5-dihydrothieno[2,3-c]pyran-7,4′-piperidine]-4-ol (S70)

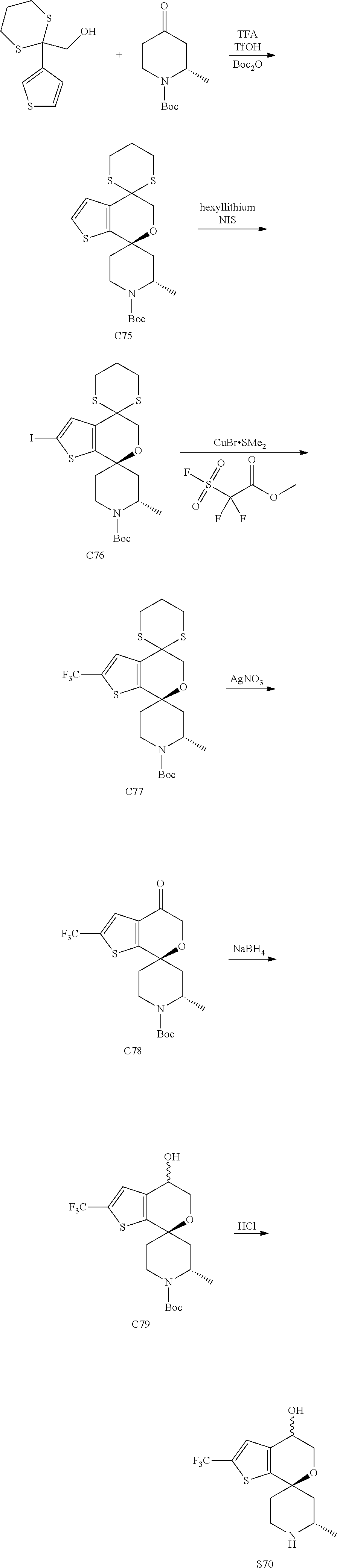

### Step 1. Synthesis of tert-butyl (6″S, 7′R)-6″-methyl-5′H-dispiro[1,3-dithiane-2,4′-thieno[2,3-c]pyran-7′,4″-piperidine]-1″-carboxylate (C75)

Tert-butyl (2S)-2-methyl-4-oxo-piperidine-1-carboxylate (2.5 g, 11.72 mmol) was dissolved in DCM (5 mL) and TFA (5 mL, 64.90 mmol) for 20 min at room temperature. The solvent was removed and the residue dissolved in dioxane (40 mL) and added to [2-(3-thienyl)-1,3-dithian-2-yl]methanol (3.0 g, 12.91 mmol). This reaction mixture was cooled to 0° C. and triflic acid (2.1 mL, 23.73 mmol) was added. The reaction mixture was warmed to room temperature. After 40 min, it was poured into EtOAc/2N NaOH (200 mL each) and the organic layer was dried and concentrated in vacuo. The crude material was then dissolved in DCM (40 mL) and Boc₂O (2.8 g, 12.83 mmol), DIPEA (5 mL, 28.71 mmol) were added. This reaction mixture was stirred at room temperature for 3 h and then washed with H₂O, dried and concentrated in vacuo, which was purified by silica gel chromatography (Gradient: 0 to 30% EtOAc in heptane) to provide tert-butyl (6″S,7′R)-6″-methyl-5′H-dispiro[1,3-dithiane-2,4′-thieno[2,3-c]pyran-7′,4″-piperidine]-1″-carboxylate (1.6 g, 32%). LCMS m/z 428.06 [M+1]⁺.

### Step 2. Synthesis of tert-butyl (6″S, 7′R)-2′-iodo-6″-methyl-5′H-dispiro[1,3-dithiane-2,4′-thieno[2,3-c]pyran-7′,4″-piperidine]-1″-carboxylate (C76)

(6″S,7′R)-6″-methyl-5′H-dispiro[1,3-dithiane-2,4′-thieno[2,3-c]pyran-7′,4″-piperidine]-1″-carboxylate C75 (800 mg, 1.871 mmol) was dissolved in THF (15 mL) and cooled to −78° C. To this solution, hexyllithium (1.6 mL of 2 M, 3.200 mmol) was added and the reaction mixture was stirred at −78° C. for 15 min, at which point NIS (420 mg, 1.867 mmol) in THF (5 mL) was added dropwise. The reaction mixture was stirred for 30 min at −78° C. then quenched with H₂O and diluted with sodium thiosulfate solution (80 ml) and EtOAc (80 ml). The organic layer was dried and concentrated in vacuo, which was purified by silica gel chromatography (Gradient: 0 to 20% EtOAc in heptane) to give the product mixed with about 40 mol % of starting material (by 1H NMR). tert-butyl (6″S,7′R)-2′-iodo-6″-methyl-5′H-dispiro[1,3-dithiane-2,4′-thieno[2,3-c]pyran-7′,4″-piperidine]-1″-carboxylate (700 mg, 41%, corrected yield). LCMS m/z 553.92 [M+1]⁺. This material was carried forward without further purification.

### Step 3. Synthesis of tert-butyl (6″S, 7′R)-6″-methyl-2′-(trifluoromethyl)-5′H-dispiro[1,3-dithiane-2,4′-thieno[2,3-c]pyran-7′,4″-piperidine]-1″-carboxylate (C77)

Tert-butyl (6″S,7′R)-2′-iodo-6″-methyl-5′H-dispiro[1,3-dithiane-2,4′-thieno[2,3-c]pyran-7′,4″-piperidine]-1″-carboxylate C76 (700 mg, 0.7588 mmol) was dissolved in DMF (5 mL) in a microwave vial with copper bromide methylsulfanylmethane (100 mg, 0.4864 mmol) and methyl 2,2-difluoro-2-fluorosulfonyl-acetate (300 μL, 2.356 mmol) added. The tube was sealed and heated to 100° C. for 50 min. The reaction mixture was diluted with EtOAc/H2O (50 mL each) and filtered through Celite®. The organic layer was dried and concentrated in vacuo, which was purified by silica gel chromatography (Gradient: 0 to 20% EtOAc in heptane) to give the product as an off-white foam: tert-butyl (6″S,7′R)-6″-methyl-2′-(trifluoromethyl)-5′H-dispiro[1,3-dithiane-2,4′-thieno[2,3-c]pyran-7′,4″-piperidine]-1″-carboxylate (180 mg, 48%). LCMS m/z 496.12 [M+1]⁺.

### Step 4. Synthesis of tert-butyl (2S,4R)-2-methyl-4′-oxo-2′-(trifluoromethyl)spiro[piperidine-4,7′-thieno[2,3-c]pyran]-1-carboxylate (C78)

Tert-butyl (6″S,7′R)-6″-methyl-2′-(trifluoromethyl)-5′H-dispiro[1,3-dithiane-2,4′-thieno[2,3-c]pyran-7′,4″-piperidine]-1″-carboxylate C77 (180 mg, 0.3305 mmol) was dissolved in EtOH (3 mL), and silver nitrate (170 mg, 1.001 mmol) dissolved in H₂O (1.5 mL) was added. The reaction mixture was heated on a sealed tube to 50° C. for 4 h. The reaction mixture was diluted with 1N NaOH/EtOAc (20 mL each) and filtered through Celite®. The organic layer was dried and concentrated to an oil, which was purified by silica gel chromatography (Gradient: 0 to 50% EtOAc in heptane) to provide the product as an oil: tert-butyl (2S,4R)-2-methyl-4′-oxo-2′-(trifluoromethyl)spiro[piperidine-4,7′-thieno[2,3-c]pyran]-1-carboxylate (70 mg, 52%). LCMS m/z 405.91 [M+1]⁺.

### Step 5. Synthesis of tert-butyl (2′S, 7R)-4-hydroxy-2′-methyl-2-(trifluoromethyl)spiro[4,5-dihydrothieno[2,3-c]pyran-7,4′-piperidine]-1′-carboxylate (C79)

Tert-butyl (2S,4R)-2-methyl-4′-oxo-2′-(trifluoromethyl)spiro[piperidine-4,7′-thieno[2,3-c]pyran]-1-carboxylate C78 (85 mg, 0.2097 mmol) was dissolved in MeOH (2 mL) and NaBH₄ (10 mg, 0.2643 mmol) was added. After 3 h, the reaction mixture was diluted with EtOAc/H₂O (20 mL each) and the organic layer was dried and concentrated in vacuo to give the product: tert-butyl (2′S,7R)-4-hydroxy-2′-methyl-2-(trifluoromethyl)spiro[4,5-dihydrothieno[2,3-c]pyran-7,4′-piperidine]-1′-carboxylate (82 mg, 96%). ¹H NMR (300 MHz, Chloroform-d) δ 7.42-7.32 (m, 1H), 4.60-4.48 (m, 1H), 4.19-3.70 (m, 4H), 3.46-3.28 (m, 1H), 2.38-2.09 (m, 3H), 2.00-1.65 (m, 2H), 1.50 (s, 9H), 1.34-1.24 (m, 3H). LCMS m/z 408.16 [M+1]⁺.

### Step 6. Synthesis of (2′S, 7R)-2′-methyl-2-(trifluoromethyl)spiro[4,5-dihydrothieno[2,3-c]pyran-7,4′-piperidine]-4-ol (S70)

Tert-butyl (2′S,7R)-4-hydroxy-2′-methyl-2-(trifluoromethyl)spiro[4,5-dihydrothieno[2,3-c]pyran-7,4′-piperidine]-1′-carboxylate C79 (82 mg, 0.2013 mmol) was dissolved in HCl (1 mL of 4 M, 4.0 mmol) in dioxane. After 30 min, the solvent was removed and the residue triturated with Et₂O to give a white solid (2′S,7R)-2′-methyl-2-(trifluoromethyl)spiro[4,5-dihydrothieno[2,3-c]pyran-7,4′-piperidine]-4-ol (Hydrochloride salt) (68 mg, 92%). ¹H NMR (300 MHz, DMSO-d₆) δ 8.94 (s, 2H), 7.62 (s, 1H), 5.60 (d, J=6.2 Hz, 1H), 4.52 (q, J=5.4 Hz, 1H), 4.05-3.89 (m, 1H), 3.76-3.60 (m, 1H), 3.26-3.03 (m, 2H), 2.36-1.75 (m, 4H), 1.33-1.17 (m, 3H). LCMS m/z 308.57 [M+1]⁺.

### Preparation S71

### (2′S,4S,7R)-2′-methyl-2-(trifluoromethyl)spiro[4,5-dihydrothieno[2,3-c]pyran-7,4′-piperidine]-4-ol (S71)

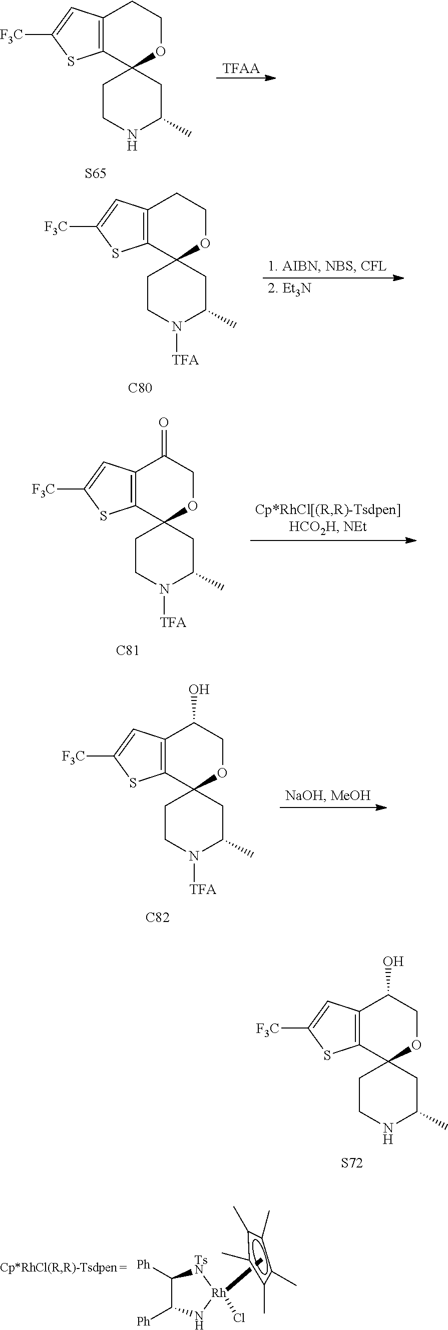

### Step 1. Synthesis of 2,2,2-trifluoro-1-[(2′S, 7R)-2′-methyl-2-(trifluoromethyl)spiro[4,5-dihydrothieno[2,3-c]pyran-7,4′-piperidine]-1′-yl]ethanone (C80)

To a mixture of (2′S,7R)-2′-methyl-2-(trifluoromethyl)spiro[4,5-dihydrothieno[2,3-c]pyran-7,4′-piperidine](2.74 g, 9.4 mmol) and DIPEA (2.0 mL, 11.5 mmol) in DCM (25 mL) cooled to 0° C. was added (2,2,2-trifluoroacetyl) 2,2,2-trifluoroacetate (1.45 mL, 10.4 mmol) dropwise. After 1 h, the mixture was quenched with 1 N HCl, and the mixture was warmed to RT. The layers were mixed and separated. The aqueous layer was extracted with DCM (×2). The organic layer was washed with brine, dried with sodium sulfate, filtered and concentrated. The crude material was purified with silica gel chromatography (Gradient: 0 to 30% EtOAc in heptane) to provide 2,2,2-trifluoro-1-[(2′S,7R)-2′-methyl-2-(trifluoromethyl)spiro[4,5-dihydrothieno[2,3-c]pyran-7,4′-piperidine]-1′-yl]ethanone (3.59 g, 95%). 1H NMR (400 MHz, CDCl3) δ 7.11 (t, J=1.2 Hz, 1H), 4.39 (s, 1H), 3.87 (t, J=5.5 Hz, 3H), 3.44 (s, 1H), 2.67 (t, J=5.5 Hz, 2H), 2.34 (ddd, J=14.4, 6.3, 1.8 Hz, 2H), 2.07-1.89 (m, 1H), 1.80 (dd, J=14.3, 10.7 Hz, 1H), 1.31 (d, J=6.5 Hz, 3H). LCMS m/z 388.25 [M+1]⁺.

### Step 2. Synthesis of (2S,4R)-2-methyl-1-(2,2,2-trifluoroacetyl)-2′-(trifluoromethyl)spiro[piperidine-4,7′-thieno[2,3-c]pyran]-4′-one (C81)

In a 250 mL flask with added 2,2,2-trifluoro-1-[(2′S,7R)-2′-methyl-2-(trifluoromethyl)spiro[4,5-dihydrothieno[2,3-c]pyran-7,4′-piperidine]-1′-yl]ethanone (3.40 g, 8.5 mmol) in MeCN (56 mL). The reaction mixture was degassed with a stream of N2 for 30 min. Then to the flask was added 1-bromopyrrolidine-2,5-dione (1.97 g, 11.1 mmol) and 2-[(E)-(1-cyano-1-methyl-ethyl)azo]-2-methyl-propanenitrile (35 mg, 0.21 mmol). Then the reaction vial was subjected under CFL (100 W) irradiation. After 5 h, the reaction mixture was quenched with aqueous 10 wt % sodium bisulfite (200 mL), stirred for 5 min, extracted with DCM (×3). The combined organic layer was washed with saturated NaHCO₃ solution and brine. The aqueous layer was back extracted with DCM (×1). Dried over MgSO4, concentrated in vacuo. Dried under vacuum to provide crude material 1-[(2′S,7R)-4-bromo-2′-methyl-2-(trifluoromethyl)spiro[4,5-dihydrothieno[2,3-c]pyran-7,4′-piperidine]-1′-yl]-2,2,2-trifluoro-ethanone. After drying overnight, the material was dissolved in DMSO (48 mL), followed by the addition of Et3N (8.3 mL, 60 mmol) in a 250 mL flask equipped with a condenser. The reaction mixture was heated to 75° C. for 3 h. The reaction mixture was cooled to RT, partitioned between MTBE, sat. aqueous NaHCO₃ solution. The organic phase was separated, aqueous layer was extracted with MTBE (×2). Combined organic phase was washed with sat. aqueous NaHCO₃ solution, dried over MgSO₄, filtered, and concentrated under reduced pressure to afford the crude material. The crude material was purified with silica gel chromatography (Gradient: 0 to 30% EtOAc in heptane) to provide (2S,4R)-2-methyl-1-(2,2,2-trifluoroacetyl)-2′-(trifluoromethyl)spiro[piperidine-4,7′-thieno[2,3-c]pyran]-4′-one (2.10 g, 58%). 1H NMR (300 MHz, CDCl3) δ 7.72 (q, J=1.2 Hz, 1H), 4.53-4.18 (m, 3H), 4.01 (s, 1H), 3.49 (s, 1H), 2.68-2.36 (m, 2H), 2.19-1.83 (m, 2H), 1.36 (d, J=6.5 Hz, 3H). LCMS m/z 402.15 [M+1]⁺.

### Step 3. Synthesis of 2,2,2-trifluoro-1-[(2′S,4S,7R)-4-hydroxy-2′-methyl-2-(trifluoromethyl)spiro[4,5-dihydrothieno[2,3-c]pyran-7,4′-piperidine]-1′-yl]ethanone (C82)

Flask A: to a 100 mL flask was added 1,2,3,4,5-pentamethylcyclopentane rhodium(II) tetrachloride (13.1 mg, 0.021 mmol) and N-[(1R,2R)-2-amino-1,2-diphenyl-ethyl]-4-methyl-benzenesulfonamide (15.8 mg, 0.043 mmol), purged with N2 for 10 min, followed by MeCN (15 mL). This catalyst mixture was stirred for 1 hr at RT. TEA (1.40 mL, 10.04 mmol) and formic acid (950 μL, 25.18 mmol) (a 5:2 commercial solution from Oakwood, 2.1 mL) were added in one portion and the reaction immediately turned a bright red and some effervescing was observed. The RBF was immediately placed into an ice bath and cooled to 0° C.

Flask B: (2S,4R)-2-methyl-1-(2,2,2-trifluoroacetyl)-2′-(trifluoromethyl)spiro[piperidine-4,7′-thieno[2,3-c]pyran]-4′-one (1.77 g, 4.16 mmol) was dissolved in MeCN (15 mL) was cooled in ice bath.

The contents of Flask B were then added to Flask A at 0° C. and the reaction was allowed to stir at 0° C. After 4 h, the reaction mixture was quenched with aqueous sat. NaHCO₃ solution, allowed to RT. The reaction mixture was extracted with MTBE (×2). The combined organic phase was washed with 1N HCl, brine, dried over sodium sulfate, filtered, and concentrated under reduced pressure. The crude material contained some water, and it was redissolved in DCM, dried over sodium sulfate, filtered and concentrated under reduced pressure. The mixture was concentrated and directly loaded onto a silica gel column for purification (0-40% EtOAc:heptane) to provide: 2,2,2-trifluoro-1-[(2′S,4S,7R)-4-hydroxy-2′-methyl-2-(trifluoromethyl)spiro[4,5-dihydrothieno[2,3-c]pyran-7,4′-piperidine]-1′-yl]ethanone (1.702 g, 100%). 1H NMR (300 MHz, CDCl3) δ 7.36 (q, J=1.2 Hz, 1H), 4.55 (dt, J=9.1, 3.1 Hz, 1H), 4.52-4.34 (m, 1H), 4.04-3.63 (m, 3H), 3.44 (s, 1H), 2.62-2.21 (m, 2H), 2.20-2.01 (m, 1H), 1.97-1.67 (m, 2H), 1.33 (d, J=6.5 Hz, 3H). LCMS m/z 404.22 [M+1]⁺.

### Step 4. Synthesis of (2′S,4S,7R)-2′-methyl-2-(trifluoromethyl)spiro[4,5-dihydrothieno[2,3-c]pyran-7,4′-piperidine]-4-ol (S72)

To 2,2,2-trifluoro-1-[(2′S,4S,7R)-4-hydroxy-2′-methyl-2-(trifluoromethyl)spiro[4,5-dihydrothieno[2,3-c]pyran-7,4′-piperidine]-1′-yl]ethanone (1.69 g, 4.17 mmol) in MeOH (15 mL) was added sodium hydroxide (12.5 mL of 1 M, 12.5 mmol). The reaction mixture was stirred at RT. After 3 h, the reaction mixture was partitioned between water and MTBE, and then organic phase was separated. Aqueous phase was extracted with MTBE (×2). Combined organic phase was washed with brine, dried over MgSO₄, filtered through medium fritted funnel, and concentrated under reduced pressure. The crude material was purified with silica gel chromatography (Gradient: 0 to 20% MeOH in DCM (VERY STREAKY!)) to provide (2′S,4S,7R)-2′-methyl-2-(trifluoromethyl)spiro[4,5-dihydrothieno[2,3-c]pyran-7,4′-piperidine]-4-ol (1.16 g, 86%). 1H NMR (300 MHz, CDCl3) δ 7.38 (q, J=1.2 Hz, 1H), 4.55 (t, J=2.9 Hz, 1H), 4.14-3.85 (m, 2H), 3.19 (dtd, J=18.5, 7.3, 6.8, 2.5 Hz, 1H), 3.07 (dd, J=12.4, 2.6 Hz, 1H), 2.96 (ddd, J=12.1, 4.8, 2.2 Hz, 1H), 2.08 (ddt, J=22.6, 13.4, 2.6 Hz, 2H), 1.80-1.40 (m, 2H), 1.11 (d, J=6.4 Hz, 3H). LCMS m/z 308.14 [M+1]⁺.

### Compound 864 and 865

### 2-[(2′S, 7R)-2′-methyl-2-(trifluoromethyl)spiro[4,5-dihydrothieno[2,3-c]pyran-7,4′-piperidine]-1′-yl]cyclopentanol (864 and 865)

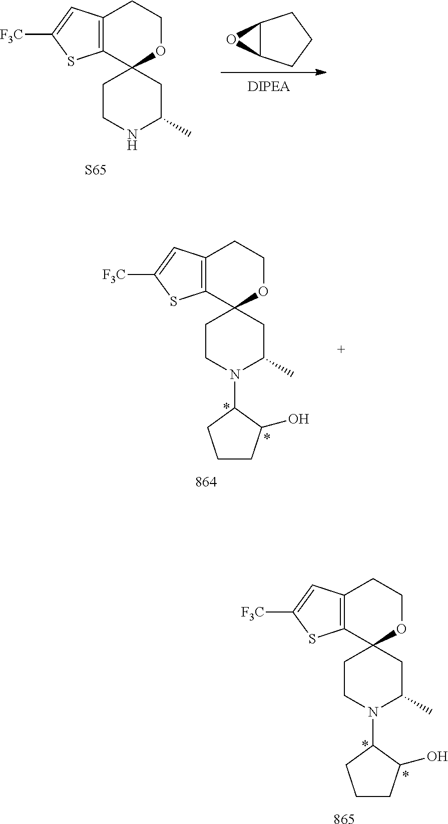

### Standard Method #A: Expoxide Opening

(2′S,7R)-2′-methyl-2-(trifluoromethyl)spiro[4,5-dihydrothieno[2,3-c]pyran-7,4′-piperidine](hydrochloride salt) S65 (60 mg, 0.1815 mmol), DIPEA (270 μL, 1.550 mmol) and 6-oxabicyclo[3.1.0]hexane (67 μL) in n-BuOH (1 ml) was heated at 210° C. for 4 h. The crude reaction mixture was evaporated. Purification by reversed-phase HPLC. Method: C18 Waters Sunfire column (30×150 mm, 5 micron). Gradient: MeCN in H₂O with 0.1% formic acid. Two products were isolated:

Compound 864: 2-[(2′S,7R)-2′-methyl-2-(trifluoromethyl)spiro[4,5-dihydrothieno[2,3-c]pyran-7,4′-piperidine]-1′-yl]cyclopentanol (Formic Acid (1)) 864 (6 mg, 15%). LCMS m/z 376.27 [M+1]⁺; and

Compound 865: 2-[(2′S,7R)-2′-methyl-2-(trifluoromethyl)spiro[4,5-dihydrothieno[2,3-c]pyran-7,4′-piperidine]-1′-yl]cyclopentanol (Formic Acid (1)) 865 (4.7 mg, 11%) LCMS m/z 376.27 [M+1]⁺.

### Compound 866

### 4-[(2′S, 7R)-2′-methyl-2-(trifluoromethyl)spiro[4,5-dihydrothieno[2,3-c]pyran-7,4′-piperidine]-1′-yl]tetrahydrofuran-3-ol (866)

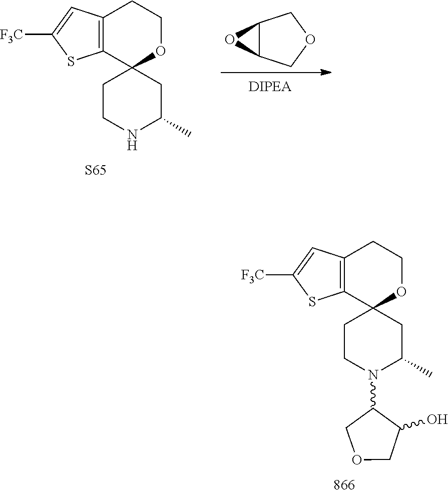

4-[(2′S,7R)-2′-methyl-2-(trifluoromethyl)spiro[4,5-dihydrothieno[2,3-c]pyran-7,4′-piperidine]-1′-yl]tetrahydrofuran-3-ol (Formic Acid (1)) S65 was prepared using standard method #A from (2′S,7R)-2′-methyl-2-(trifluoromethyl)spiro[4,5-dihydrothieno[2,3-c]pyran-7,4′-piperidine](Hydrochloride salt) and 3,6-dioxabicyclo[3.1.0]hexane. ¹H NMR (300 MHz, Methanol-d₄) 8.43 (s, 1H), 7.29 (t, J=1.2 Hz, 1H), 4.60 (td, J=5.3, 4.4, 1.6 Hz, 1H), 4.22-4.09 (m, 2H), 4.04-3.92 (m, 2H), 3.53 (dd, J=9.9, 4.4 Hz, 1H), 3.31 (dq, J=3.3, 1.7 Hz, 2H), 3.13 (td, J=12.5, 2.8 Hz, 1H), 2.99 (ddd, J=12.3, 4.6, 2.4 Hz, 1H), 2.72 (t, J=5.5 Hz, 2H), 2.38-2.19 (m, 2H), 2.17-1.81 (m, 2H), 1.30 (d, J=6.5 Hz, 3H). LCMS m/z 378.26 [M+1]⁺.

### Compound 867

### (2S)-2-hydroxy-3-[(2′S, 7R)-2′-methyl-2-(trifluoromethyl)spiro[4,5-dihydrothieno[2,3-c]pyran-7,4′-piperidine]-1′-yl]propenamide (867)

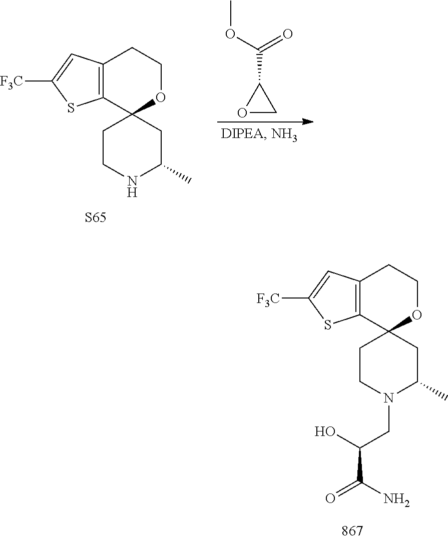

(2′S,7R)-2′-methyl-2-(trifluoromethyl)spiro[4,5-dihydrothieno[2,3-c]pyran-7,4′-piperidine]S65 (88 mg, 0.2599 mmol) and methyl (2S)-oxirane-2-carboxylate (500 μL, 5.711 mmol) were added to a microwave vial and brought up in NH₃ (2.5 mL of 7 M, 17.50 mmol) in MeOH (2.5 mL). DIPEA (700 μL, 4.019 mmol) was added and reaction stirred at 120° C. for 3 h. The solution was transferred to a different microwave vial. An additional methyl (2S)-oxirane-2-carboxylate (300 μL) and NH₃/MeOH (500 μL) were added and reaction was stirred at 120° C. for 2 h. The reaction was quenched with water and citric acid until pH reached 6. Organics were extracted with EtOAc (4×) and concentrated down in vacuo. Purification by silica gel chromatography (Gradient: 0-20% MeOH in DCM) afforded the product mixed with impurities. Further purification by reversed-phase HPLC. Method: C18 Waters Sunfire column (30×150 mm, 5 micron). Gradient: MeCN in H2O with 0.1% trifluoroacetic acid. Product was isolated as (2S)-2-hydroxy-3-[(2′S,7R)-2′-methyl-2-(trifluoromethyl)spiro[4,5-dihydrothieno[2,3-c]pyran-7,4′-piperidine]-1′-yl]propanamide (Trifluoroacetate salt) (39.7 mg, 31%). ¹H NMR indicated a mixture of epimers (85:15). ¹H NMR (300 MHz, Chloroform-d) δ 7.32 (s, 1H), 7.14 (s, 1H), 6.29 (s, 1H), 4.73 (dd, J=10.1, 2.7 Hz, 1H), 4.07-3.82 (m, 3H), 3.72 (d, J=12.0 Hz, 1H), 3.53 (s, 1H), 3.33 (d, J=9.6 Hz, 1H), 2.95 (t, J=11.9 Hz, 1H), 2.73 (q, J=5.6, 5.1 Hz, 2H), 2.52-2.26 (m, 2H), 2.18 (t, J=13.1 Hz, 2H), 1.51 (d, J=6.4 Hz, 3H). LCMS m/z 379.21 [M+1]⁺.

### Compound 868

### (2S)-2-hydroxy-3-[(2′S, 7R)-2′-methyl-2-(trifluoromethyl)spiro[4,5-dihydrothieno[2,3-c]pyran-7,4′-piperidine]-1′-yl]propanoic acid (868)

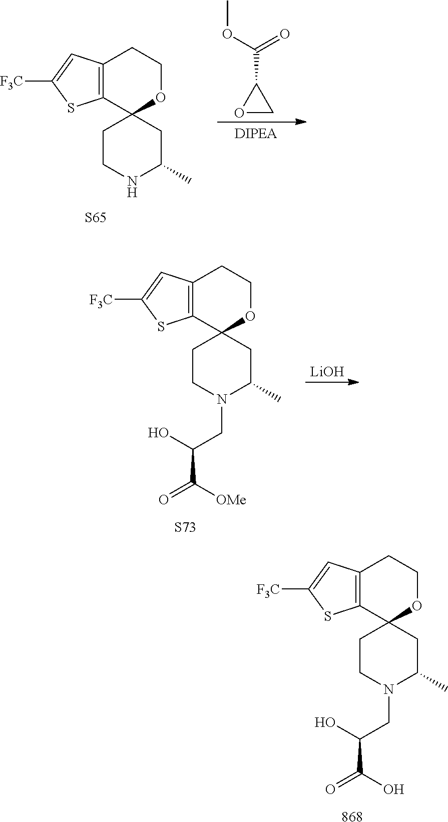

### Step 1. Synthesis of methyl (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoate (S73)

(2′S,7R)-2′-methyl-2-(trifluoromethyl)spiro[4,5-dihydrothieno[2,3-c]pyran-7,4′-piperidine](132.3 mg, 0.4541 mmol) S65 and methyl (2S)-oxirane-2-carboxylate (120 μL, 1.371 mmol) were added to a microwave flask and brought up in MeOH (2.6 mL). DIPEA (400 μL, 2.296 mmol) was added and the reaction was stirred at 90° C. for 3 h. The reaction mixture was concentrated down in vacuo and rinsed with MeOH/DCM (3×) to provide product methyl (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoate (244 mg, 100%). LCMS m/z 394.13 [M+1]⁺.

### Step 2. Synthesis of (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoic acid (868)

Methyl (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoate S73 (244 mg, 0.6202 mmol) and LiOH (42.9 mg, 1.791 mmol) were added to a round bottom flask. Then the reaction mixture was dissolved in MeOH (3 mL), THF (3 mL) and the reaction mixture was allowed to stir at room temperature for 4 h. The reaction mixture was concentrated in vacuo. Purification by reversed-phase HPLC. Method: C18 Waters Sunfire column (30×150 mm, 5 micron). Gradient: MeCN in H₂O with 0.1% trifluoroacetic acid. Product was isolated as (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoic acid (84.3 mg, 36%). ¹H NMR (300 MHz, Chloroform-d) δ 10.98 (s, 1H), 7.16-7.14 (m, 1H), 4.80 (d, J=9.3 Hz, 1H), 3.95 (td, J=11.0, 5.4 Hz, 3H), 3.73 (d, J=12.0 Hz, 1H), 3.60 (s, 1H), 3.39 (t, J=12.5 Hz, 1H), 3.09 (dd, J=13.9, 9.5 Hz, 1H), 2.73 (q, J=5.1 Hz, 2H), 2.45-2.26 (m, 1H), 2.23 (s, 1H), 2.18 (s, 1H), 1.50 (d, J=6.4 Hz, 3H). Material is a 90:10 mixture of epimers based on ¹H NMR. LCMS m/z 380.25 [M+1]⁺.

### Compound 869

### (2S)-2-hydroxy-N-methyl-3-[(2′S, 7R)-2′-methyl-2-(trifluoromethyl)spiro[4,5-dihydrothieno[2,3-c]pyran-7,4′-piperidine]-1′-yl]propanamide (869)

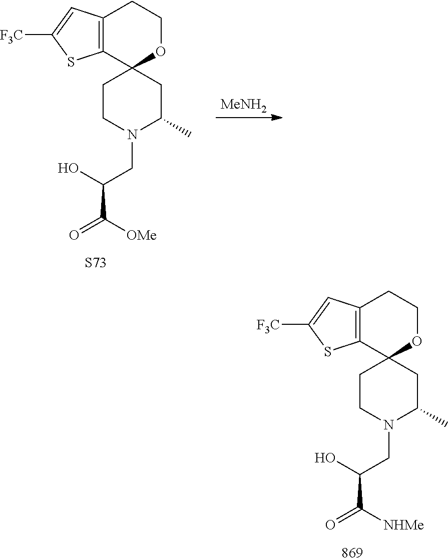

Methyl (2S)-2-hydroxy-3-[(2′S,7R)-2′-methyl-2-(trifluoromethyl)spiro[4,5-dihydrothieno[2,3-c]pyran-7,4′-piperidine]-1′-yl]propanoate S73 (70 mg, 0.1779 mmol) was dissolved in dioxane (1.4 mL) in a microwave flask. Methyl amine (180 μL of 40% w/v, 2.318 mmol) and water (30 μL, 1.665 mmol) were added and the reaction was heated to 80° C. for 3 h, then stirred at room temperature overnight. Reaction mixture was concentrated down via rotovap. Purification by reversed-phase HPLC. Method: C18 Waters Sunfire column (30×150 mm, 5 micron). Gradient: MeCN in H₂O with 0.1% trifluoroacetic acid. Product was isolated as (2S)-2-hydroxy-N-methyl-3-[(2′S,7R)-2′-methyl-2-(trifluoromethyl)spiro[4,5-dihydrothieno[2,3-c]pyran-7,4′-piperidine]-1′-yl]propanamide (Trifluoroacetate salt) (40.8 mg, 41%). ¹H NMR (300 MHz, Chloroform-d) δ 7.39 (s, 1H), 7.16 (s, 1H), 4.72 (dd, J=10.1, 2.9 Hz, 1H), 4.08 (d, J=13.7 Hz, 1H), 3.96 (p, J=6.0 Hz, 2H), 3.71 (d, J=12.2 Hz, 1H), 3.53 (s, 1H), 3.37 (t, J=11.8 Hz, 1H), 2.91 (dd, J=10.7, 5.1 Hz, 3H), 2.75 (d, J=3.2 Hz, 2H), 2.55-2.29 (m, 2H), 2.19 (t, J=14.2 Hz, 2H), 1.52 (d, J=6.5 Hz, 3H). Material is an 85:15 mixture of epimers based on ¹H NMR. LCMS m/z 393.21 [M+1]⁺.

### Compound 870

### (2′S, 7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine](870)

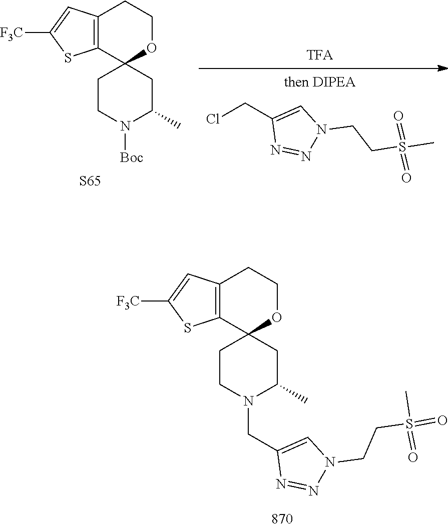

Tert-butyl (2′S,7R)-2′-methyl-2-(trifluoromethyl)spiro[4,5-dihydrothieno[2,3-c]pyran-7,4′-piperidine]-1′-carboxylate S65 (30 mg, 0.07664 mmol) was dissolved in DCM (2 mL) and TFA (1 mL, 12.98 mmol) was added. After 20 min, the solvent was removed and the residue dissolved in DCE (1 mL) and added to 4-(chloromethyl)-1-(2-methylsulfonylethyl)triazole (25 mg, 0.1118 mmol). DIPEA (30 μL, 0.1722 mmol) was then added and the reaction mixture heated to 60° C. in a sealed tube. After 24 h, additional 4-(chloromethyl)-1-(2-methylsulfonylethyl)triazole (15 mg, 0.06706 mmol) was added and heating continued for 24 h. The reaction mixture was purified by silica gel chromatography (Gradient: 0-10% MeOH in DCM) yielded the product. Further purification by reverse phase HPLC (C18, TFA modifier, 15.5 g). Pure fractions combined and diluted with 1N NaOH/EtOAc and the organic layer was dried and concentrated to give the product as a white solid. (2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine](14 mg, 36%) ¹H NMR (300 MHz, Chloroform-d) δ 7.68 (s, 1H), 7.10 (s, 1H), 4.87 (t, J=6.4 Hz, 2H), 4.13-4.02 (m, 1H), 3.90-3.82 (m, 2H), 3.79-3.67 (m, 2H), 2.80-2.53 (m, 8H), 2.12-1.97 (m, 3H), 1.97-1.82 (m, 1H), 1.71 (t, J=12.7 Hz, 1H), 1.28-1.20 (m, 3H). LCMS m/z 479.15 [M+1]⁺.

### Compound 871

### [(2′S, 7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-yl]methanol

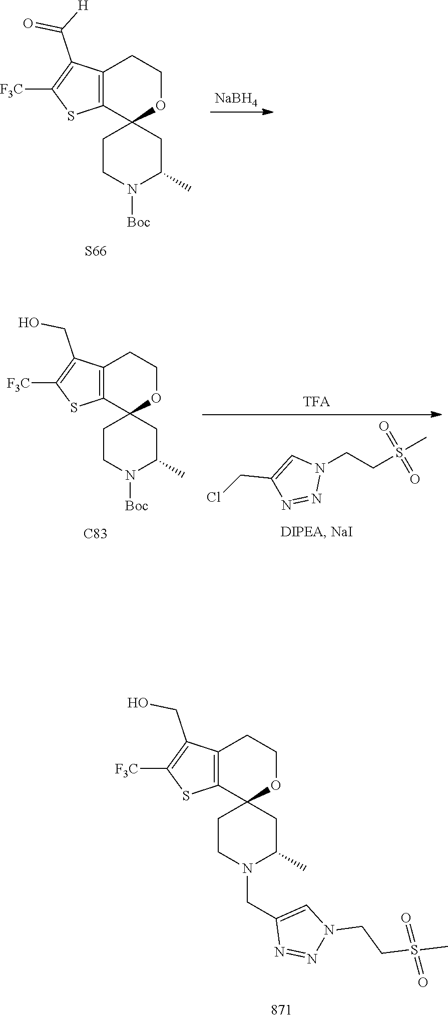

### Step 1. Synthesis of tert-butyl (2′S, 7R)-3-(hydroxymethyl)-2′-methyl-2-(trifluoromethyl)spiro[4,5-dihydrothieno[2,3-c]pyran-7,4′-piperidine]-1′-carboxylate (C83)

Tert-butyl (2′S,7R)-3-formyl-2′-methyl-2-(trifluoromethyl)spiro[4,5-dihydrothieno[2,3-c]pyran-7,4′-piperidine]-1′-carboxylate S66 (45 mg, 0.1073 mmol) was dissolved in MeOH (3 mL) and NaBH₄ (10 mg, 0.2643 mmol) was added. After 15 min, the reaction mixture was diluted with EtOAc and H₂O. The organic layer was dried and concentrated to an oil, which was purified by silica chromatography (Gradient: 0 to 60% EtOAc/heptane, 12 g silica) to give the product as a colorless oil. Tert-butyl (2′S,7R)-3-(hydroxymethyl)-2′-methyl-2-(trifluoromethyl)spiro[4,5-dihydrothieno[2,3-c]pyran-7,4′-piperidine]-1′-carboxylate (32 mg, 71%). ¹H NMR (300 MHz, Chloroform-d) δ 4.70-4.63 (m, 2H), 4.10-3.97 (m, 1H), 3.94 (td, J=5.7, 1.5 Hz, 2H), 3.83-3.69 (m, 1H), 3.43-3.28 (m, 1H), 2.77-2.67 (m, 2H), 2.31-2.08 (m, 2H), 1.88-1.61 (m, 3H), 1.51 (s, 9H), 1.28 (d, J=6.5 Hz, 3H). LCMS m/z 422.65 [M+1]⁺.

### Step 2. Synthesis of [(2′S, 7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-yl]methanol (871)

Tert-butyl (2′S,7R)-3-(hydroxymethyl)-2′-methyl-2-(trifluoromethyl)spiro[4,5-dihydrothieno[2,3-c]pyran-7,4′-piperidine]-1′-carboxylate C83 (32 mg, 0.07592 mmol) was dissolved in DCM (1 mL) and TFA (0.5 mL, 6.490 mmol). After 15 min, the solvent was removed and the residue was redissolved in DCE (0.5 mL) and 4-(chloromethyl)-1-(2-methylsulfonylethyl)triazole (hydrochloride salt) (25 mg, 0.1112 mmol), sodium iodide (3 mg, 0.02001 mmol) and DIPEA (80 μL, 0.4593 mmol) were added. The reaction mixture was heated to 60° C. in a sealed tube for 24 h. The reaction mixture was concentrated, dissolved in MeOH and load on a C18 column (15.5 g) and eluted with CH₃CN/H₂O with TFA modifier. Fractions containing product were combined, diluted with EtOAc/1N NaOH and the organic layer was dried, concentrated and further purified by silica chromatography (0 to 15% MeOH/DCM, 4 g silica) to give the product as a colorless oil. [(2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-yl]methanol (9.7 mg, 25%) ¹H NMR (300 MHz, Chloroform-d) δ 7.66 (s, 1H), 4.88 (t, J=6.3 Hz, 2H), 4.66 (d, J=1.3 Hz, 2H), 4.06 (d, J=14.7 Hz, 1H), 4.03-3.84 (m, 3H), 3.75 (t, J=6.3 Hz, 2H), 2.84-2.49 (m, 7H), 2.20-1.83 (m, 5H), 1.69 (dd, J=13.9, 11.3 Hz, 1H), 1.26 (d, J=6.2 Hz, 3H). LCMS m/z 509.13 [M+1]⁺.

### Compound 872

### [(2′S,7R)-2′-methyl-1′-[[1-(2-methylsulfonylethyl)pyrazol-4-yl]methyl]-2-(trifluoromethyl)spiro[4,5-dihydrothieno[2,3-c]pyran-7,4′-piperidine]-3-yl]methanol (872)

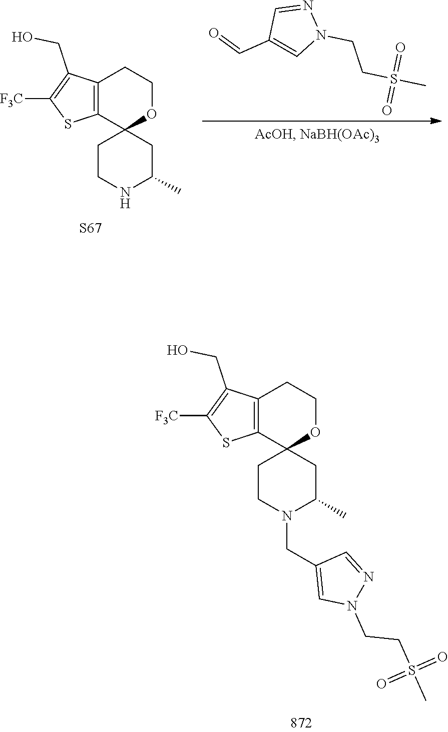

To a solution of [(2′S,7R)-2′-methyl-2-(trifluoromethyl)spiro[4,5-dihydrothieno[2,3-c]pyran-7,4′-piperidine]-3-yl]methanol S67 (20 g, 62.05 mmol) and 1-(2-methylsulfonylethyl)pyrazole-4-carbaldehyde (13.6 g, 67.25 mmol) in THF (350 mL) was added AcOH (4 mL, 70.34 mmol) followed by sodium triacetoxyborohydride (30 g, 141.5 mmol). The resulting suspension was warmed to 55° C. After 2 h, the reaction was cooled in ice water bath, and then quenched with 1 M aqueous HCl (˜350 mL) until pH=1. Stirred at room temperature for 25 min, then extracted with MTBE (˜400 mL), MTBE extract was discarded. The aqueous solution was treated with DCM (˜400 mL), with stirring the aqueous layer was basified to pH 11 via the addition of 2 M aqueous NaOH (˜270 mL). Layers were separated, and the aqueous layer was extracted with DCM (2×100 mL). Organic extracts were dried with MgSO₄, filtered and concentrated. The crude residue was treated with MTBE (570 mL), and the foam went into solution. As it solubilized, crystals were observed and immediately filtered. Collected crystals were washed with MTBE (2×60 mL), dried under suction for 20 min to afford desired product 29.2 g as a white crystalline solid. The material was treated with IPA (80 mL), heated to reflux for 30 min, then it was allowed to slowly cool to room temperature, stirred overnight. After 14 h, the slurry was filtered, collected solid was washed with IPA (2×20 mL). Collected solid was dried under suction for 2 h, then transferred to rotovap. The material was dried at 30 mbar, bath temp was slowly raised from 25° C. to 70° C. over the course of 1 h. The solid was dried further in vacuum oven at 90° C. for 14 h to afford [(2′S,7R)-2′-methyl-1′-[[1-(2-methylsulfonylethyl)pyrazol-4-yl]methyl]-2-(trifluoromethyl)spiro[4,5-dihydrothieno[2,3-c]pyran-7,4′-piperidine]-3-yl]methanol (27 g, 85%) as a white crystalline solid. ¹H NMR (400 MHz, DMSO-d₆) δ 7.72 (d, J=0.7 Hz, 1H), 7.40 (d, J=0.7 Hz, 1H), 5.23 (t, J=5.3 Hz, 1H), 4.51 (t, J=6.8 Hz, 2H), 4.43 (dd, J=5.4, 1.5 Hz, 2H), 3.83 (q, J=5.9 Hz, 2H), 3.75-3.61 (m, 3H), 3.48 (s, 1H), 2.77 (s, 3H), 2.63 (t, J=5.6 Hz, 2H), 2.60-2.53 (m, 1H), 2.47-2.29 (m, 2H), 2.00-1.89 (m, 2H), 1.74-1.63 (m, 1H), 1.47 (dd, J=13.5, 11.3 Hz, 1H), 1.10 (d, J=6.1 Hz, 3H). ¹⁹F NMR (376 MHz, DMSO-d₆) δ−51.03. LCMS m/z 508.24 [M+1]⁺.

### Compound 873

### [(2′S, 7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (873)

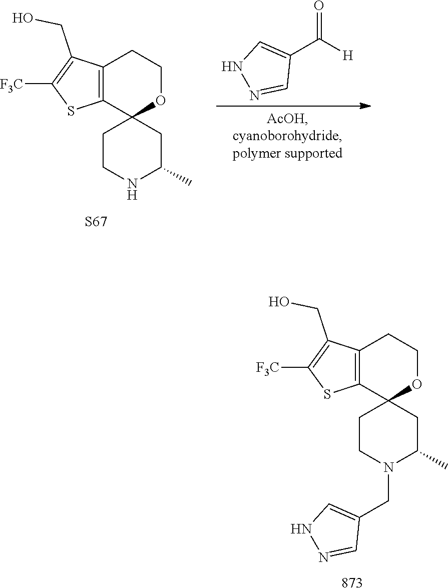

### Standard Method #B1: Reductive Amination

[(2′S,7R)-2′-methyl-2-(trifluoromethyl)spiro[4,5-dihydrothieno[2,3-c]pyran-7,4′-piperidine]-3-yl]methanol S67 (44 mg, 0.137 mmol) was added in a microwave vial and 1H-pyrazole-4-carbaldehyde (26.3 mg, 0.274 mmol), acetic acid (37.5 μL, 0.659 mmol) and DCM (1.9 mL) were added. Cyanoborohydride, polymer supported (211 mg, 0.422 mmol) was added. The solution was capped and heated to 95° C. in a microwave reactor for 120 minutes. MeOH was added and the solution was stirred for 10 minutes to wash the beads. The solutions were filtered, and the solvent removed in vacuo. Purification by reversed-phase HPLC. Method: C18 Waters Sunfire column (30×150 mm, 5 micron). Gradient: MeCN in H₂O with 10 mM ammonium hydroxide. Product was isolated as [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (9.3 mg, 17%). ¹H NMR (300 MHz, Methanol-d₄) δ 7.59 (s, 2H), 4.57 (s, 2H), 4.10-3.74 (m, 3H), 3.66 (d, J=14.2 Hz, 1H), 2.85-2.41 (m, 5H), 2.20-1.54 (m, 4H), 1.24 (d, J=6.3 Hz, 3H). LCMS m/z 402.19 [M+1]⁺.

### Alternative Route to Prepare Compound 873

### [(2′S, 7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (873)

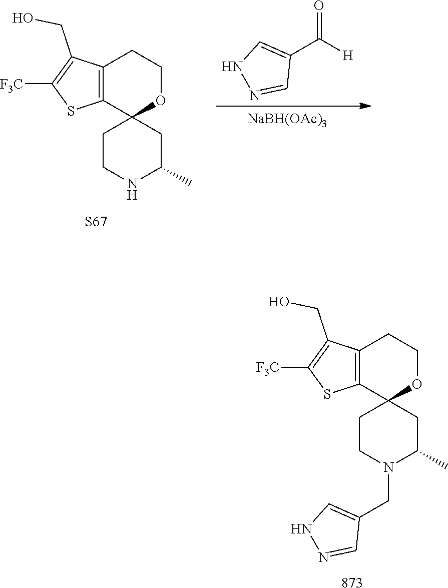

Standard Method #B2: Reductive Amination with Sodium Triacetoxyborohydride

To a solution of 1H-pyrazole-4-carbaldehyde (600 mg, 6.244 mmol) in THF (15 mL) was added [(2′S,7R)-2′-methyl-2-(trifluoromethyl)spiro[4,5-dihydrothieno[2,3-c]pyran-7,4′-piperidine]-3-yl]methanol S67 (1 g, 3.112 mmol), sodium triacetoxyborohydride (887 mg, 4.205 mmol) was added. The mixture was stirred for 45 min at 50° C. It was then cooled in ice-water bath and quenched with saturated aqueous sodium bicarbonate solution, then EtOAc was added. The aqueous layer was extracted with EtOAc, and the organic layer was washed with brine. The organic layer was separated and dried with Na₂SO₄, filtered, and the solvent was removed in vacuo. The crude material was purified by silica gel chromatography (Gradient: 0-20% MeOH in DCM) to yield [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (1.05 g, 82%). LCMS m/z 402.1 [M+1]⁺.

### Compound 874

### (2′S,4S,7R)-2′-methyl-1′-(1H-pyrazol-4-ylmethyl)-2-(trifluoromethyl)spiro[4,5-dihydrothieno[2,3-c]pyran-7,4′-piperidine]-4-ol (874)

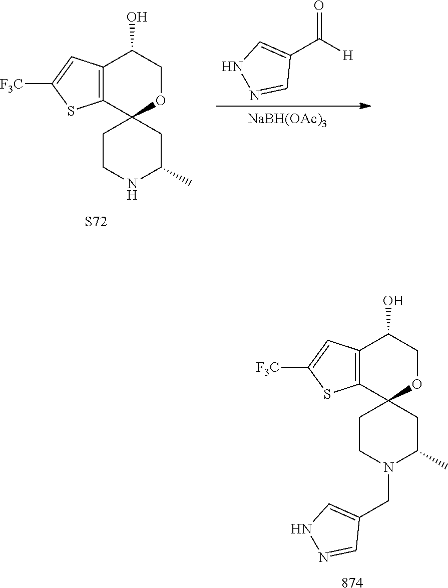

To a solution of (2′S,4S,7R)-2′-methyl-2-(trifluoromethyl)spiro[4,5-dihydrothieno[2,3-c]pyran-7,4′-piperidine]-4-ol S72 (1.0 g, 3.17 mmol) and 1H-pyrazole-4-carbaldehyde (333 mg, 3.47 mmol) in THF (14 mL) was added sodium triacetoxyboranuide (1.46 g, 6.89 mmol) and the mixture was heated to 55° C. After 3.5 h, the mixture was cooled to RT, diluted with ethyl acetate and quenched with sat. aq. sodium bicarbonate. The organic layer was separated, washed with brine, dried with magnesium sulfate, filtered, and concentrated. The mixture was minimally diluted in DCM and loaded onto a silica gel column for purification (0-20% MeOH:DCM) to provide desired product, containing about 35 mol % MeOH. The solid was heated under vacuum to 55° C. for 20 h, which lowered the MeOH content to 4.5 mol % (and a very small amount of DCM). Final product was isolated as (2′S,4S,7R)-2′-methyl-1′-(1H-pyrazol-4-ylmethyl)-2-(trifluoromethyl)spiro[4,5-dihydrothieno[2,3-c]pyran-7,4′-piperidine]-4-ol (1.12 g, 87%). 1H NMR (300 MHz, CDCl3) δ 7.53 (s, 2H), 7.34 (q, J=1.2 Hz, 1H), 4.50 (t, J=2.7 Hz, 1H), 4.08-3.79 (m, 3H), 3.59 (d, J=14.2 Hz, 1H), 2.83-2.59 (m, 2H), 2.45 (td, J=12.3, 2.6 Hz, 1H), 2.10 (dd, J=14.1, 3.0 Hz, 1H), 2.00 (dt, J=13.6, 2.9 Hz, 1H), 1.82-1.69 (m, 2H), 1.23 (d, J=6.2 Hz, 3H). LCMS m/z 388.2 [M+1]⁺.

### Compounds 875-886

Compounds 875-886 (see Table 38) were prepared in a single step from intermediate S67, S69, S70, and S72 using standard method #B1 or B2. Aldehydes were obtained from commercial sources or described previously. Any modifications to methods are noted in Table 38 and accompanying footnotes.

TABLE 38
Structure and physicochemical data for compounds 875-886

| | | Aldehyde | Starting | ¹H NMR; LCMS m/z |
|---|---|---|---|---|
| Cmpd | Structure | Reagent | MaterMial | [M + H]⁺ |
| 875 | 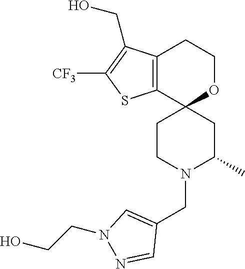 [SMILES: C[C@H]1C[C@@]2(CCN1Cc1cnn(CCO)c1)OCCc1c2sc(C(F)(F)F)c1CO] | 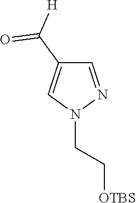 [SMILES: CC(C)(C)[Si](C)(C)OCCn1cc(C=O)cn1] | S67¹ | ¹H NMR (300 MHz, Chloroform-d) δ 7.44 (s, 1H), 7.36 (s, 1H), 4.73- 4.57 (m, 2H), 4.21 (dd, J = 5.6, 3.9 Hz, 2H), 4.06- 3.79 (m, 5H), 3.50 (d, J = 14.2 Hz, 1H), 3.24 (s, 1H), 2.71 (td, J = 5.5, 1.7 Hz, 3H), 2.65-2.40 (m, 2H), 2.02 (ddt, J = 11.5, 6.5, 2.9 Hz, 2H), 1.84 (td, J = 12.8, 4.5 Hz, 2H), 1.76- 1.59 (m, 1H), 1.19 (d, J = 6.1 Hz, 3H); LCMS m/z 446.07 [M + H]⁺ |
| 876 | 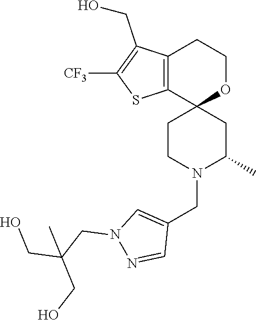 [SMILES: C[C@H]1C[C@@]2(CCN1Cc1cnn(CC(C)(CO)CO)c1)OCCc1c2sc(C(F)(F)F)c1CO] | 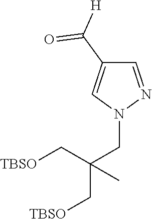 | S67¹ | ¹H NMR (300 MHz, Methanol-d₄) δ 7.63 (s, 1H), 7.46 (d, J = 3.9 Hz, 1H), 4.57 (s, 2H), 4.14 (d, J = 3.8 Hz, 2H), 3.86 (d, J = 24.2 Hz, 3H), 3.67 (d, J = 14.3 Hz, 1H), 3.38 (t, J = 4.2 Hz, 4H), 2.65 (d, J = 31.3 Hz, 5H), 2.21-1.53 (m, 4H), 1.23 (d, J = 5.8 Hz, 3H), 0.83 (q, J = 2.5 Hz, 3H); LCMS m/z 504.15 [M + H]⁺ |
| 877 | 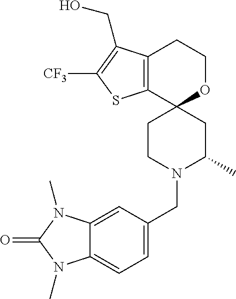 [SMILES: C[C@H]1C[C@@]2(CCN1Cc1ccc3c(c1)n(C)c(=O)n3C)OCCc1c2sc(C(F)(F)F)c1CO] | 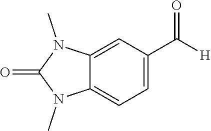 | S67² | ¹H NMR (300 MHz, Chloroform-d) δ 7.14- 6.97 (m, 2H), 6.92 (d, J = 7.9 Hz, 1H), 4.66 (d, J = 4.5 Hz, 2H), 4.26 (d, J = 12.9 Hz, 1H), 3.98 (qt, J = 11.7, 5.6 Hz, 2H), 3.45 (d, J = 7.9 Hz, 6H), 3.14 (d, J = 12.9 Hz, 1H), 2.85- 2.57 (m, 4H), 2.51-2.25 (m, 1H), 2.13-1.94 (m, 3H), 1.89-1.68 (m, 2H), 1.25 (d, J = 6.2 Hz, 3H); LCMS m/z 496.2 [M + H]⁺ |
| 878 | 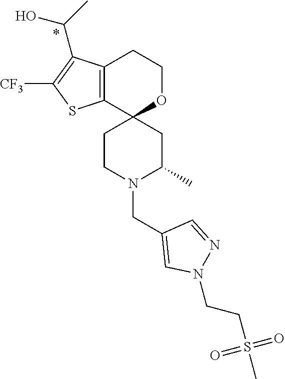 [SMILES: CC(O)c1c(C(F)(F)F)sc2c1CCO[C@@]21CCN(Cc2cnn(CCS(C)(=O)=O)c2)[C@@H](C)C1] | 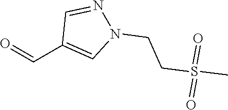 | S69³ | ¹H NMR (400 MHz, Chloroform-d) δ 7.52 (d, J = 0.8 Hz, 1H), 7.50 (s, 1H), 5.31-5.19 (m, 1H), 4.68-4.52 (m, 2H), 3.96- 3.76 (m, 3H), 3.72-3.60 (m, 3H), 3.00 (dt, J = 16.3, 4.7 Hz, 1H), 2.83-2.66 (m, 2H), 2.61 (s, 1H), 2.54 (d, J = 11.4 Hz, 1H), 2.48 (s, 3H), 2.08-2.01 (m, 2H), 1.90 (dd, J = 13.1, 4.3 Hz, 1H), 1.72 (d, J = 12.7 Hz, 1H), 1.51 (d, J = 6.7 Hz, 3H), 1.23 (d, J = 6.2 Hz, 3H); LCMS m/z 522.19 [M + H]⁺ |
| 879 | 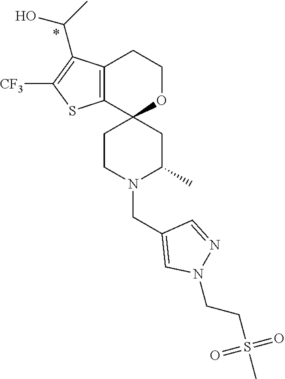 [SMILES: CC(O)c1c(C(F)(F)F)sc2c1CCO[C@@]21CCN(Cc2cnn(CCS(C)(=O)=O)c2)[C@@H](C)C1] | 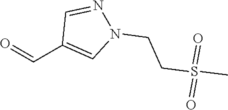 | S69³ | ¹H NMR (400 MHz, Chloroform-d) δ 7.53 (s, 1H), 7.51 (s, 1H), 5.26 (q, J = 6.7 Hz, 1H), 4.66- 4.57 (m, 2H), 3.94-3.81 (m, 3H), 3.74-3.58 (m, 3H), 2.98 (dt, J = 16.4, 5.6 Hz, 1H), 2.75 (dq, J = 16.3, 5.3 Hz, 2H), 2.55 (t, J = 12.7 Hz, 2H), 2.49 (s, 3H), 2.10-1.99 (m, 2H), 1.93 (t, J = 12.9 Hz, 1H), 1.52 (d, J = 6.7 Hz, 3H), 1.28 (t, J = 3.6 Hz, 1H), 1.24 (dd, J = 6.1, 2.0 Hz, 3H); LCMS m/z 522.14 M + H]⁺ |
| 880 | 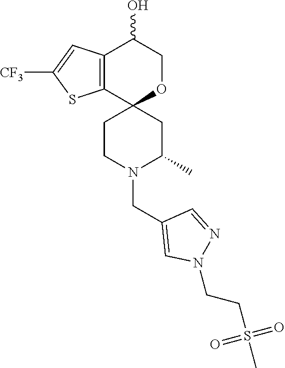 | 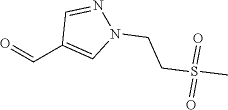 | S70⁴ | ¹H NMR (300 MHz, Chloroform-d) δ 7.52 (s, 1H), 7.47 (s, 1H), 7.36 (s, 1H), 4.62 (t, J = 6.0 Hz, 2H), 4.53 (d, J = 8.3 Hz, 1H), 4.02-3.78 (m, 3H), 3.73-3.50 (m, 3H), 2.82- 2.49 (m, 3H), 2.48 (s, 3H), 2.17-1.90 (m, 4H), 1.86- 1.70 (m, 1H), 1.26-1.17 (m, 3H); LCMS m/z 494.27 [M + H]⁺ |
| 881 | 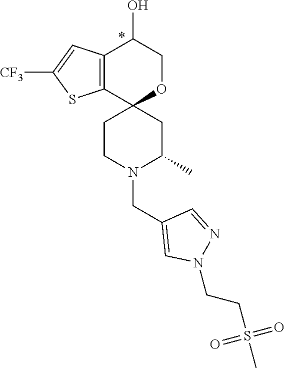 | 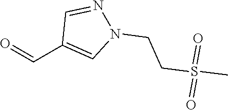 | S70^{4,5} | ¹H NMR (300 MHz, Chloroform-d) δ 7.43 (s, 1H), 7.39 (s, 1H), 7.27 (s, 1H), 4.60-4.48 (m, 2H), 4.43 (t, J = 2.7 Hz, 1H), 3.87-3.74 (m, 3H), 3.64- 3.46 (m, 3H), 2.67-2.46 (m, 2H), 2.40 (s, 4H), 2.13- 1.78 (m, 3H), 1.78-1.58 (m, 2H), 1.14 (d, J = 6.2 Hz, 3H); LCMS m/z 494.19 [M + H]⁺ |
| 882 | 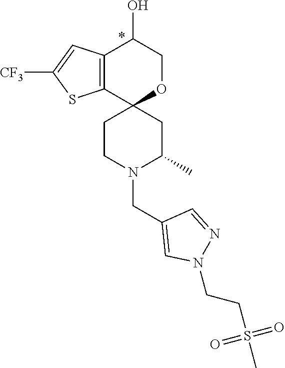 | 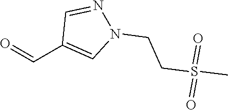 | S70^{4,5} | ¹H NMR (300 MHz, Chloroform-d) δ 7.51 (s, 1H), 7.47 (s, 1H), 7.37 (s, 1H), 4.61 (t, J = 6.0 Hz, 2H), 4.54 (t, J = 3.2 Hz, 1H), 4.02-3.74 (m, 3H), 3.74-3.52 (m, 3H), 2.81- 2.64 (m, 1H), 2.64-2.43 (m, 5H), 2.16-1.92 (m, 4H), 1.67-1.45 (m, 1H), 1.21 (d, J = 6.2 Hz, 3H); LCMS m/z 494.19 [M + H]⁺ |
| 883 | 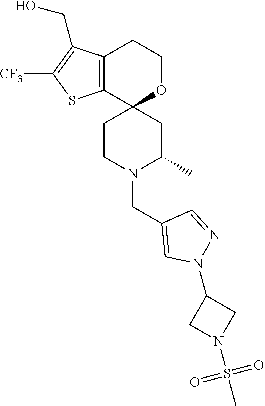 [SMILES: C[C@H]1C[C@@]2(CCN1Cc1cnn(C3CN(S(C)(=O)=O)C3)c1)OCCc1c2sc(C(F)(F)F)c1CO] | 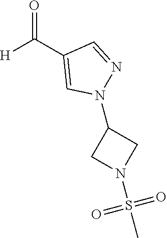 | S67⁶ | 1H NMR (300 MHz, MeOD) δ 7.93 (s, 1H), 7.73 (s, 1H), 5.28 (t, J = 7.1 Hz, 1H), 4.59 (s, 2H), 4.46-4.24 (m, 5H), 4.05 (d, J = 14.4 Hz, 1H), 3.96 (d, J = 3.0 Hz, 2H), 3.24- 3.00 (m, 3H), 3.07 (s, 3H), 2.75 (t, J = 5.5 Hz, 2H), 2.26 (t, J = 14.2 Hz, 2H), 2.07-1.77 (m, 2H), 1.44 (d, J = 6.4 Hz, 3H); LCMS m/z 535.34 [M + H]⁺ |
| 884 | 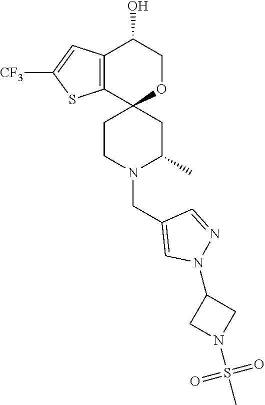 [SMILES: C[C@H]1C[C@@]2(CCN1Cc1cnn(C3CN(S(C)(=O)=O)C3)c1)OC[C@@H](O)c1cc(C(F)(F)F)sc12] | 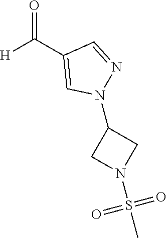 | S72⁶ | 1H NMR (300 MHz, MeOD) δ 7.94 (s, 1H), 7.74 (s, 1H), 7.48 (s, 1H), 5.36-5.21 (m, 1H), 4.56 (t, J = 3.7 Hz, 1H), 4.44- 4.32 (m, 6H), 4.15-4.03 (m, 1H), 3.97 (dd, J = 12.2, 3.4 Hz, 1H), 3.85- 3.75 (m, 1H), 3.45-3.35 (m, 1H), 3.25-3.09 (m, 1H), 3.07 (s, 3H), 2.32 (d, J = 14.7 Hz, 2H), 1.94 (d, J = 13.6 Hz, 2H), 1.46 (d, J = 6.3 Hz, 3H); LCMS m/z 521.34 [M + H]⁺ |
| 885 | 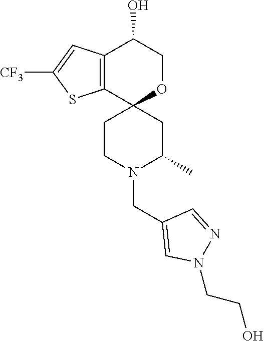 [SMILES: C[C@H]1C[C@@]2(CCN1Cc1cnn(CCO)c1)OC[C@@H](O)c1cc(C(F)(F)F)sc12] | 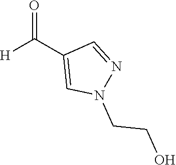 | S72⁶ | 1H NMR (300 MHz, MeOD) δ 8.40 (s, 1H), 7.88 (s, 1H), 7.66 (d, J = 0.8 Hz, 1H), 7.49 (q, J = 1.2 Hz, 1H), 4.56 (t, J = 3.6 Hz, 1H), 4.45 (d, J = 14.1 Hz, 1H), 4.26 (t, J = 5.2 Hz, 2H), 4.14 (d, J = 14.0 Hz, 1H), 4.06-3.76 (m, 4H), 3.59-3.39 (m, 1H), 3.29-3.02 (m, 3H), 2.46-2.22 (m, 2H), 2.10- 1.85 (m, 2H), 1.50 (d, J = 6.4 Hz, 3H); LCMS m/z 432.22 [M + H]⁺ |
| 886 | 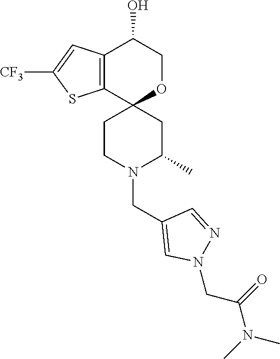 [SMILES: C[C@H]1C[C@@]2(CCN1Cc1cnn(CC(=O)N(C)C)c1)OC[C@@H](O)c1cc(C(F)(F)F)sc12] | 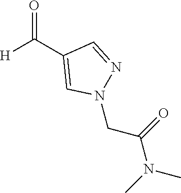 | S72⁶ | 1H NMR (300 MHz, MeOD) δ 8.38 (s, 1H), 7.83 (s, 1H), 7.64 (s, 1H), 7.49 (d, J = 1.3 Hz, 1H), 5.18 (s, 2H), 4.57 (t, J = 3.7 Hz, 1H), 4.46 (d, J = 14.0 Hz, 1H), 4.10 (d, J = 14.0 Hz, 1H), 3.99 (dd, J = 12.3, 3.4 Hz, 1H), 3.83 (dd, J = 12.3, 3.9 Hz, 1H), 3.13 (s, 5H), 2.98 (s, 3H), 2.37 (t, J = 15.6 Hz, 2H), 2.11-1.83 (m, 2H), 1.49 (d, J = 6.4 Hz, 3H); LCMS m/z 473.26 [M + H]⁺ |

Footnotes:
¹HCl was added to remove the TBS protecting groups at the end of reaction.
²0.1% trifluoroacetic acid modifier was used in the reversed-phase purification.
³The stoichiometry of the reagents was modified as below: 1.5 eq of the corresponding aldehyde, 0.24 eq of acetic acid and 2.0 eq of cyanoborohydride, polymer supported. The reaction was performed at 85° C. overnight.
⁴The stoichiometry of the reagents was modified as below: 2.0 eq of the corresponding aldehyde, and 2.2 eq of cyanoborohydride, polymer supported. The reaction was performed at 85° C. for 16 h. The product was isolated using silica gel chromatography (Gradient: 0 to 100% EtOAc in heptane, 1% Et₃N).
⁵The enantiomer was obtained from 880 by chiral SFC separation. Column: Daicel Chiralpak ® IB, 10 × 250 mm; Mobile Phase: 20% MeOH (containing 5 mM ammonia), 80% carbon dioxide. Flow: 15 mL/min.
⁶Following the standard procedure B2 with sodium triacetoxyborohydride as the reductant.

### Compound 887

### (2S)-1-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (887)

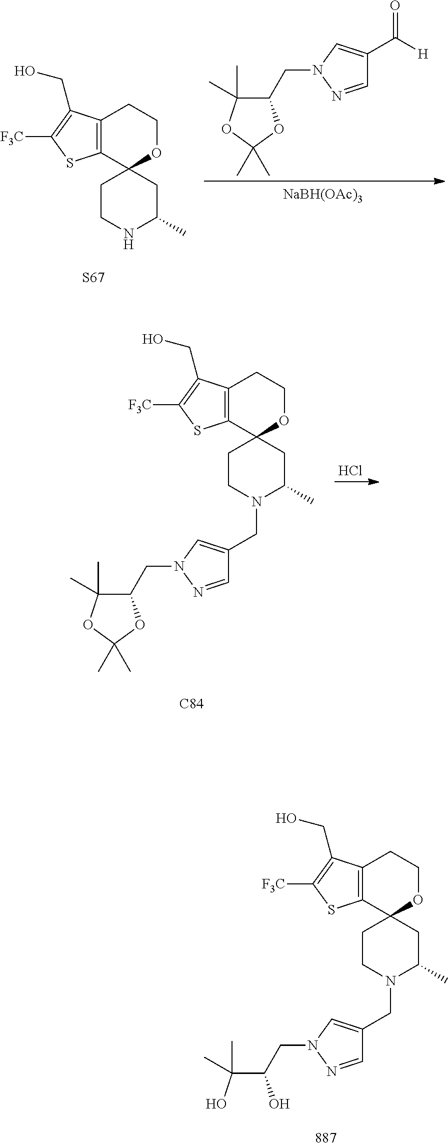

### Step 1: Synthesis of [(2′S, 7R)-2′-methyl-1′-[[1-[[(4S)-2,2,5,5-tetramethyl-1,3-dioxolan-4-yl]methyl]pyrazol-4-yl]methyl]-2-(trifluoromethyl)spiro[4,5-dihydrothieno[2,3-c]pyran-7,4′-piperidine]-3-yl]methanol (C84)

To a solution of [(2′S,7R)-2′-methyl-2-(trifluoromethyl)spiro[4,5-dihydrothieno[2,3-c]pyran-7,4′-piperidine]-3-yl]methanol (41 mg, 0.1199 mmol) and 1-[[(4S)-2,2,5,5-tetramethyl-1,3-dioxolan-4-yl]methyl]pyrazole-4-carbaldehyde (31 mg, 0.1301 mmol) in THF (500 μL) was added sodium triacetoxyborohyride (55 mg, 0.2595 mmol) and the mixture was heated to 50° C. for 2.5 h. The mixture was cooled to rt, diluted with ethyl acetate and quenched with sat. aq. sodium bicarbonate. The organic layer was separated, washed with brine, dried with magnesium sulfate, filtered, and concentrated. The mixture was purified with silica gel chromatography (0-100% EtOAc/EtOH 3:1 in heptane) to provide [(2′S,7R)-2′-methyl-1′-[[1-[[(4S)-2,2,5,5-tetramethyl-1,3-dioxolan-4-yl]methyl]pyrazol-4-yl]methyl]-2-(trifluoromethyl)spiro[4,5-dihydrothieno[2,3-c]pyran-7,4′-piperidine]-3-yl]methanol (59.6 mg, 78%). LCMS m/z 544.36 [M+1]⁺.

### Step 2: Synthesis of (2S)-1-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (887)

To [(2′S,7R)-2′-methyl-1′-[[1-[[(4S)-2,2,5,5-tetramethyl-1,3-dioxolan-4-yl]methyl]pyrazol-4-yl]methyl]-2-(trifluoromethyl)spiro[4,5-dihydrothieno[2,3-c]pyran-7,4′-piperidine]-3-yl]methanol (59.6 mg, 0.1020 mmol) in a 20 mL vial was added THF (500 μL), followed by hydrogen chloride (85 μL of 6 M, 0.5100 mmol). The reaction was stirred at RT for 20 h. The reaction mixture was concentrated and purification by reversed-phase HPLC. Method: Waters XSelect CSH C18 OBD Prep Column; 30×150 mm, 5 micron. Gradient: Acetonitrile in Water with 0.1% Trifluoroacetic Acid. The product was isolated as (2S)-1-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (trifluoroacetic acid salt) (51.6 mg, 68%). 1H NMR (300 MHz, CDCl3) δ 11.56 (s, 1H), 7.78 (s, 1H), 7.57 (s, 1H), 4.65 (s, 2H), 4.60 (d, J=13.9 Hz, 1H), 4.40 (dd, J=13.9, 2.4 Hz, 1H), 4.14 (dd, J=13.8, 9.4 Hz, 1H), 4.02-3.71 (m, 4H), 3.46 (d, J=12.0 Hz, 2H), 3.04 (s, 1H), 2.81-2.69 (m, 2H), 2.37-2.22 (m, 2H), 2.22-2.06 (m, 2H), 1.59 (d, J=6.4 Hz, 3H), 1.29 (d, J=7.4 Hz, 6H). LCMS m/z 504.41 [M+1]⁺.

### Compound 888

### (2R)-1-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (888)

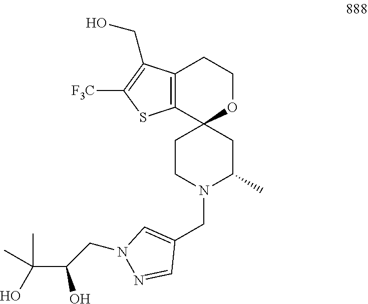 [SMILES: C[C@H]1C[C@@]2(CCN1Cc1cnn(C[C@@H](O)C(C)(C)O)c1)OCCc1c2sc(C(F)(F)F)c1CO]

The title compound was prepared in the same manner as compound 887 from [(2′S,7R)-2′-methyl-2-(trifluoromethyl)spiro[4,5-dihydrothieno[2,3-c]pyran-7,4′-piperidine]-3-yl]methanol and 1-[[(4R)-2,2,5,5-tetramethyl-1,3-dioxolan-4-yl]methyl]pyrazole-4-carbaldehyde. 1H NMR (300 MHz, MeOD) δ 7.97-7.86 (m, 1H), 7.69 (s, 1H), 4.66-4.53 (m, 3H), 4.49 (dd, J=13.8, 2.3 Hz, 1H), 4.27 (d, J=14.0 Hz, 1H), 4.17-3.89 (m, 3H), 3.72 (dd, J=9.9, 2.3 Hz, 1H), 3.66-3.49 (m, 1H), 3.38 (d, J=5.7 Hz, 2H), 2.77 (t, J=5.5 Hz, 2H), 2.51-2.26 (m, 2H), 2.16-1.89 (m, 2H), 1.55 (d, J=6.4 Hz, 3H), 1.25 (d, J=7.5 Hz, 6H). LCMS m/z 504.37 [M+1]⁺.

### Compound 889

### (2S)-1-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (889)

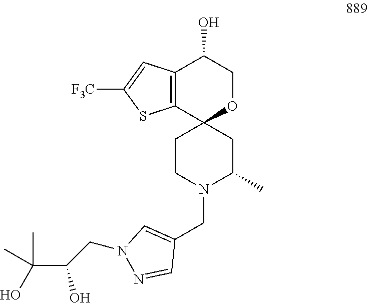 [SMILES: C[C@H]1C[C@@]2(CCN1Cc1cnn(C[C@H](O)C(C)(C)O)c1)OC[C@@H](O)c1cc(C(F)(F)F)sc12]

The title compound was prepared in the same manner as compound 887 from (2′S,4S,7R)-2′-methyl-2-(trifluoromethyl)spiro[4,5-dihydrothieno[2,3-c]pyran-7,4′-piperidine]-4-ol (S72) and 1-[[(4S)-2,2,5,5-tetramethyl-1,3-dioxolan-4-yl]methyl]pyrazole-4-carbaldehyde. 1H NMR (300 MHz, MeOD) δ 7.92 (s, 1H), 7.69 (s, 1H), 7.50 (d, J=1.3 Hz, 1H), 4.67-4.55 (m, 2H), 4.50 (dd, J=13.8, 2.3 Hz, 1H), 4.28 (d, J=14.1 Hz, 1H), 4.13-3.93 (m, 2H), 3.83 (dd, J=12.3, 3.8 Hz, 1H), 3.70 (dd, J=9.9, 2.2 Hz, 1H), 3.62 (s, 1H), 3.41 (d, J=11.0 Hz, 2H), 2.44 (t, J=16.6 Hz, 2H), 2.12-1.86 (m, 2H), 1.56 (d, J=6.4 Hz, 3H), 1.25 (d, J=7.6 Hz, 6H). LCMS m/z 490.37 [M+1]⁺.

### Compound 890

### (2R)-1-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]-3-methyl-butane-2,3-diol (890)

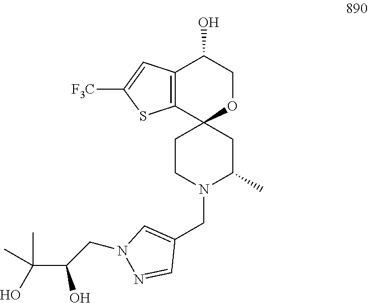 [SMILES: C[C@H]1C[C@@]2(CCN1Cc1cnn(C[C@@H](O)C(C)(C)O)c1)OC[C@@H](O)c1cc(C(F)(F)F)sc12]

The title compound was prepared in the same manner as compound 887 (2′S,4S,7R)-2′-methyl-2-(trifluoromethyl)spiro[4,5-dihydrothieno[2,3-c]pyran-7,4′-piperidine]-4-ol (S72) and 1-[[(4R)-2,2,5,5-tetramethyl-1,3-dioxolan-4-yl]methyl]pyrazole-4-carbaldehyde. 1H NMR (300 MHz, MeOD) δ 7.96-7.88 (m, 1H), 7.69 (s, 1H), 7.50 (d, J=1.2 Hz, 1H), 4.58 (dd, J=8.6, 5.0 Hz, 2H), 4.49 (dd, J=13.9, 2.3 Hz, 1H), 4.28 (d, J=14.0 Hz, 1H), 4.16-3.94 (m, 2H), 3.84 (dd, J=12.3, 3.8 Hz, 1H), 3.72 (dd, J=9.8, 2.3 Hz, 1H), 3.62 (s, 1H), 3.46-3.32 (m, 2H), 2.44 (t, J=16.2 Hz, 2H), 2.13-1.84 (m, 2H), 1.56 (d, J=6.5 Hz, 3H), 1.25 (d, J=7.6 Hz, 6H). LCMS m/z 490.37 [M+1]⁺.

### Preparation S74

### tert-butyl-dimethyl-[[(2′S, 7R)-2′-methyl-1′-(1H-pyrazol-4-ylmethyl)-2-(trifluoromethyl)spiro[4,5-dihydrothieno[2,3-c]pyran-7,4′-piperidine]-3-yl]methoxy]silane (S74)

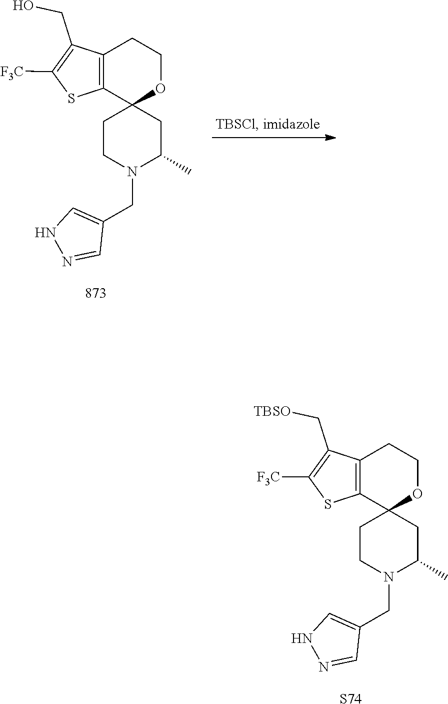 [SMILES: C[C@H]1C[C@@]2(CCN1Cc1cn[nH]c1)OCCc1c2sc(C(F)(F)F)c1CO ; C[C@H]1C[C@@]2(CCN1Cc1cn[nH]c1)OCCc1c2sc(C(F)(F)F)c1CO[Si](C)(C)C(C)(C)C ; "S74"]

To a flask with [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol 873 (1.05 g, 2.563 mmol) was added DCM (21 mL), imidazole (175 mg, 2.571 mmol) and TBSCl (1.15 mL, 6.608 mmol). The reaction mixture was stirred at room temperature for 3 h. The mixture was diluted 1 N HCl (30 mL) and DCM. The aqueous layer was separated, and the organic layer was washed with brine. The combined organic layer was concentrated. Purification by silica gel chromatography (Gradient: 0-20% MeOH in DCM) yielded the product. Tert-butyl-dimethyl-[[(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methoxy]silane (700 mg, 53%). ¹H NMR (300 MHz, Chloroform-d) δ 7.54 (s, 2H), 4.64 (d, J=1.3 Hz, 2H), 4.02-3.76 (m, 3H), 3.61 (d, J=14.2 Hz, 1H), 2.82-2.41 (m, 5H), 2.03 (ddt, J=11.0, 5.6, 2.8 Hz, 2H), 1.95-1.79 (m, 1H), 1.70 (t, J=12.6 Hz, 1H), 1.22 (d, J=6.2 Hz, 3H), 0.89 (s, 9H), 0.06 (s, 6H). LCMS m/z 516.14 [M+1]⁺.

### Compound 891

### 1-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butan-2-ol (891)

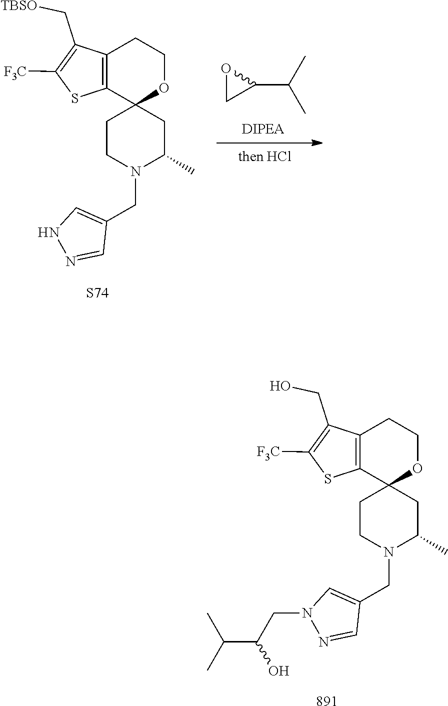

### Standard Method #C: Epoxide Opening

Tert-butyl-dimethyl-[[(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methoxy]silane S74 (50 mg, 0.0968 mmol) and 2-isopropyloxirane (16.7 mg, 0.194 mmol) were added to a microwave vial and dissolved in MeOH (850 μL). DIPEA (84 μL, 0.483 mmol) was added and the reaction was heated for 3.5 h at 120° C. HCl (4N in dioxane, 0.5 ml) was added to the reaction mixture and the reaction mixture was stirred for 30 min. The solvent was removed and purified by reversed-phase HPLC. Method: C18 Waters Sunfire column (30×150 mm, 5 micron) with 10 mM ammonium hydroxide. Product was isolated as 1-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-3-methyl-butan-2-ol (3.7 mg, 8%). ¹H NMR (300 MHz, Methanol-d₄) δ 7.63 (s, 1H), 7.46 (s, 1H), 4.57 (d, J=1.4 Hz, 2H), 4.23 (dd, J=14.0, 3.6 Hz, 1H), 4.08-3.79 (m, 4H), 3.76-3.54 (m, 2H), 2.84-2.48 (m, 5H), 2.05 (d, J=12.3 Hz, 2H), 1.86 (dt, J=14.0, 6.9 Hz, 1H), 1.71-1.51 (m, 2H), 1.23 (d, J=6.2 Hz, 3H), 0.99 (dd, J=6.8, 4.3 Hz, 6H). LCMS m/z 488.21 [M+1]⁺.

### Compounds 892-895

Compounds 892-895 (see Table 39) were prepared in a single step from intermediate S74 or 873 using standard method #C as in the preparation of compound 891. Epoxides were obtained from commercial sources. Any modifications to methods are noted in Table 39 and accompanying footnotes.

TABLE 38
Structure and physicochemical data for compounds 892-895

| | | Epoxide | Starting | ¹H NMR; LCMS m/z |
|---|---|---|---|---|
| Cmpd | Structure | Reagent | material | [M + H]⁺ |
| 892 | 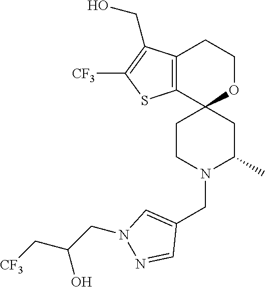 [SMILES: C[C@H]1C[C@@]2(CCN1Cc1cnn(CC(O)CC(F)(F)F)c1)OCCc1c2sc(C(F)(F)F)c1CO] | 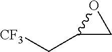 | S74 | ¹H NMR (300 MHz, Methanol-d₄) δ 7.70 (s, 1H), 7.53 (s, 1H), 4.62- 4.52 (m, 2H), 4.42-4.10 (m, 3H), 4.10-3.83 (m, 3H), 3.76-3.61 (m, 1H), 2.95-2.62 (m, 5H), 2.43- 2.05 (m, 4H), 1.89 (td, J = 13.3, 4.5 Hz, 1H), 1.71 (t, J = 12.8 Hz, 1H), 1.29 (d, J = 6.3 Hz, 3H); LCMS m/z 528.17 [M + H]⁺ |
| 892 | 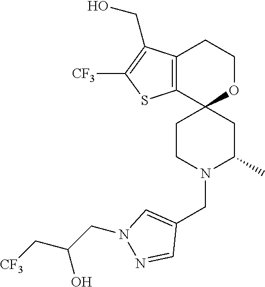 [SMILES: C[C@H]1C[C@@]2(CCN1Cc1cnn(CC(O)CC(F)(F)F)c1)OCCc1c2sc(C(F)(F)F)c1CO] | 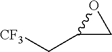 | S74 | ¹H NMR (300 MHz, Methanol-d₄) δ 7.70 (s, 1H), 7.53 (s, 1H), 4.62- 4.52 (m, 2H), 4.42-4.10 (m, 3H), 4.10-3.83 (m, 3H), 3.76-3.61 (m, 1H), 2.95-2.62 (m, 5H), 2.43- 2.05 (m, 4H), 1.89 (td, J = 13.3, 4.5 Hz, 1H), 1.71 (t, J = 12.8 Hz, 1H), 1.29 (d, J = 6.3 Hz, 3H); LCMS m/z 528.17 [M + H]⁺ |
| 893 | 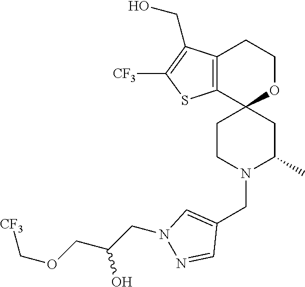 [SMILES: C[C@H]1C[C@@]2(CCN1Cc1cnn(CC(O)COCC(F)(F)F)c1)OCCc1c2sc(C(F)(F)F)c1CO] | 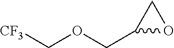 | S74 | ¹H NMR (300 MHz, Methanol-d₄) δ 7.62 (s, 1H), 7.48 (s, 1H), 4.57 (d, J = 1.2 Hz, 2H), 4.39- 3.45 (m, 11H), 2.91- 2.44 (m, 5H), 2.19-1.52 (m, 4H), 1.23 (d, J = 6.3 Hz, 3H); LCMS m/z 558.15 [M + H]⁺ |
| 894 | 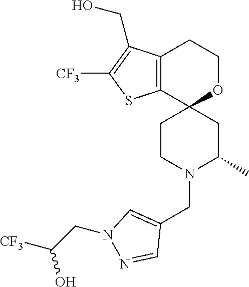 [SMILES: C[C@H]1C[C@@]2(CCN1Cc1cnn(CC(O)C(F)(F)F)c1)OCCc1c2sc(C(F)(F)F)c1CO] | 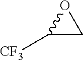 | S74 | ¹H NMR (300 MHz, Methanol-d₄) δ 7.70 (s, 1H), 7.53 (s, 1H), 4.58 (d, J = 1.4 Hz, 2H), 4.52- 4.14 (m, 3H), 4.10-3.80 (m, 3H), 3.67 (dd, J = 14.3, 2.5 Hz, 1H), 2.85- 2.35 (m, 5H), 2.21-1.63 (m, 4H), 1.26 (d, J = 6.3 Hz, 3H); LCMS m/z 514.17 [M + H]⁺ |
| 895 | 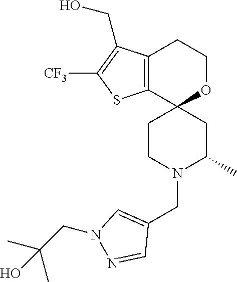 [SMILES: C[C@H]1C[C@@]2(CCN1Cc1cnn(CC(C)(C)O)c1)OCCc1c2sc(C(F)(F)F)c1CO] | 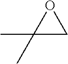 | 873¹ | ¹H NMR (300 MHz, Chloroform-d) δ 7.77 (s, 1H), 7.56 (s, 1H), 4.58 (s, 2H), 4.25-3.84 (m, 6H), 3.14-2.61 (m, 5H), 2.19 (d, J = 14.2 Hz, 2H), 1.96 (ddd, J = 17.3, 12.9, 6.3 Hz, 1H), 1.81 (t, J = 13.2 Hz, 1H), 1.37 (d, J = 6.3 Hz, 3H), 1.16 (s, 6H); LCMS m/z 474.2 [M + H]⁺ |

Footnotes:
¹2,2-dimethyloxirane (1 eq) was used. HCl deprotection was not performed, because the starting material did not have a silyl protecting group. The reaction was heated at 85° C. for 16 h.

### Compound 896

### 3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propanamide (896)

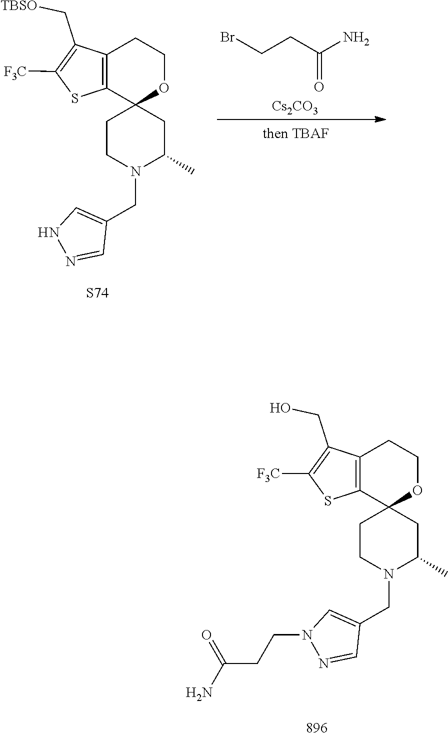 [SMILES: C[C@H]1C[C@@]2(CCN1Cc1cn[nH]c1)OCCc1c2sc(C(F)(F)F)c1CO[Si](C)(C)C(C)(C)C ; "Cs2CO3" ; NC(=O)CCBr ; "S74" ; C[C@H]1C[C@@]2(CCN1Cc1cnn(CCC(N)=O)c1)OCCc1c2sc(C(F)(F)F)c1CO ; "896"]

To tert-butyl-dimethyl-[[(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methoxy]silane S74 (30 mg, 0.05386 mmol) in a 1-dram vial was added 3-bromopropanamide (16 mg, 0.1053 mmol), followed by cesium carbonate (53 mg, 0.1627 mmol) and DMF (200 μL). The reaction mixture was heated to 80° C. After 2 h, the reaction mixture was diluted with 0.4 mL of DMF and treated with tetrabutylammonium (Fluoride Ion (1)) (110 μL of 1 M, 0.1100 mmol). After 90 min, the reaction was concentrated to remove excess THF and purified by reversed-phase HPLC. Method: C18 Waters Sunfire column (30×150 mm, 5 micron) with 10 mM ammonium hydroxide. Product was isolated as 3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propanamide (13.8 mg, 53%). ¹H NMR (300 MHz, Methanol-d₄) δ 7.60 (s, 1H), 7.46 (s, 1H), 4.58 (d, J=1.4 Hz, 2H), 4.41 (t, J=6.6 Hz, 2H), 3.92 (td, J=5.7, 2.4 Hz, 2H), 3.84 (d, J=14.3 Hz, 1H), 3.60 (d, J=14.2 Hz, 1H), 2.85-2.66 (m, 5H), 2.66-2.47 (m, 2H), 2.22-1.96 (m, 2H), 1.85 (td, J=13.2, 4.4 Hz, 1H), 1.65 (dd, J=14.1, 11.5 Hz, 1H), 1.23 (d, J=6.2 Hz, 3H). LCMS m/z 473.21 [M+1]⁺.

### Compounds 897-910

Compounds 897-910 (see Table 40) were prepared in a single step from intermediate S74 or 873 using method described in the preparation of 896. Alkyl halides were obtained from commercial sources. Any modifications to methods are noted in Table 40 and accompanying footnotes.

TABLE 40
Structure and physicochemical data for compounds 897-910

| | | Alkyl Halide | Starting | ¹H NMR; LCMS m/z |
|---|---|---|---|---|
| Cmpd | Structure | Reagent | Material | [M + H]⁺ |
| 897 | 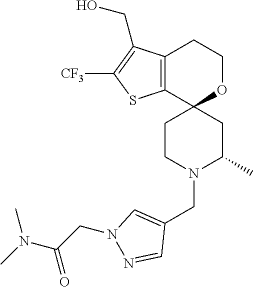 | 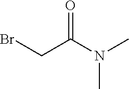 | S74¹ | ¹H NMR (300 MHz, Chloroform-d) δ 7.48 (d, J = 7.3 Hz, 2H), 4.97 (s, 2H), 4.64 (s, 2H), 3.90 (hept, J = 6.0 Hz, 3H), 3.57 (s, 1H), 3.08 (s, 3H), 2.99 (s, 3H), 2.86-2.35 (m, 5H), 2.02 (s, 2H), 1.88 (s, 1H), 1.22 (s, 4H), 0.83 (s, 1H); LCMS m/z 487.18 [M + H]⁺ |
| 898 | 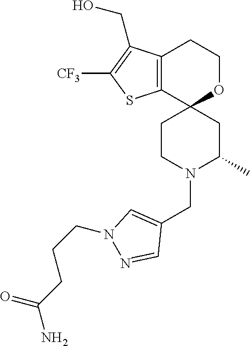 [SMILES: C[C@H]1C[C@@]2(CCN1Cc1cnn(CCCC(N)=O)c1)OCCc1c2sc(C(F)(F)F)c1CO] | 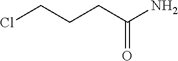 | S74² | ¹H NMR (300 MHz, Methanol-d₄) δ 7.63 (s, 1H), 7.47 (s, 1H), 4.58 (d, J = 1.3 Hz, 2H), 4.19 (t, J = 6.4 Hz, 2H), 4.09-3.77 (m, 3H), 3.64 (d, J = 14.2 Hz, 1H), 2.81-2.47 (m, 5H), 2.34-1.99 (m, 6H), 1.86 (td, J = 13.2, 4.5 Hz, 1H), 1.65 (dd, J = 14.0, 11.5 Hz, 1H), 1.24 (d, J = 6.3 Hz, 3H); LCMS m/z 487.16 [M + H]⁺ |
| 899 | 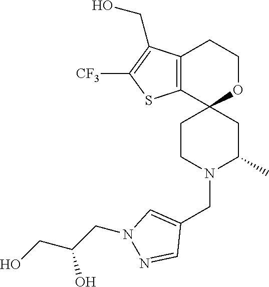 [SMILES: C[C@H]1C[C@@]2(CCN1Cc1cnn(C[C@H](O)CO)c1)OCCc1c2sc(C(F)(F)F)c1CO] | 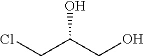 | S74³ | 1H NMR (300 MHz, MeOD) δ 7.97 (s, 1H), 7.75 (d, J = 3.9 Hz, 1H), 4.69-4.52 (m, 3H), 4.47- 4.12 (m, 3H), 3.98 (h, J = 7.0, 6.1 Hz, 3H), 3.58 (s, 1H), 3.51 (d, J = 5.3 Hz, 2H), 3.37 (d, J = 5.5 Hz, 2H), 2.77 (t, J = 5.5 Hz, 2H), 2.51-2.24 (m, 2H), 2.08 (q, J = 15.2, 14.3 Hz, 2H), 1.56 (d, J = 6.4 Hz, 3H); LCMS m/z 476.13 [M + H]⁺ |
| 900 | 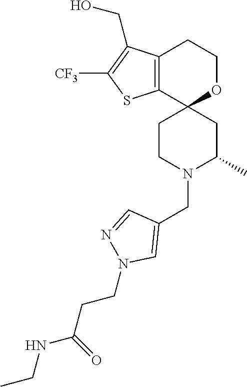 [SMILES: CCNC(=O)CCn1cc(CN2CC[C@]3(C[C@@H]2C)OCCc2c3sc(C(F)(F)F)c2CO)cn1] | 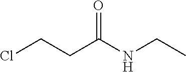 | S74³ | 1H NMR (400 MHz, DMSO) δ 10.87 (s, 1H), 7.76 (t, J = 6.3 Hz, 1H), 7.69 (s, 1H), 7.44 (s, 1H), 4.25 (s, 2H), 4.14 (q, J = 6.5 Hz, 3H), 3.96 (dd, J = 14.2, 5.3 Hz, 1H), 3.69 (ddt, J = 16.6, 12.0, 5.7 Hz, 2H), 3.02 (dd, J = 32.6, 12.9 Hz, 2H), 2.91- 2.72 (m, 3H), 2.56-2.44 (m, 2H), 2.41 (t, J = 6.7 Hz, 2H), 2.15-1.87 (m, 4H), 1.26 (d, J = 6.3 Hz, 3H), 0.76 (t, J = 7.2 Hz, 3H); LCMS m/z 501.22 [M + H]⁺ |
| 901 | 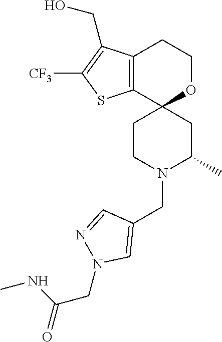 | 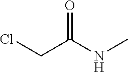 | S74³ | 1H NMR (300 MHz, MeOD) δ 7.96 (s, 1H), 7.72 (s, 1H), 4.90 (s, 2H), 4.62 (d, J = 12.7 Hz, 3H), 4.24 (d, J = 14.0 Hz, 1H), 3.99 (q, J = 5.4 Hz, 2H), 3.62 (s, 1H), 3.39 (d, J = 14.8 Hz, 2H), 2.78 (s, 5H), 2.48-2.26 (m, 2H), 2.13-1.94 (m, 2H), 1.56 (d, J = 6.5 Hz, 3H); LCMS m/z 473.12 M + H]⁺ |
| 902 | 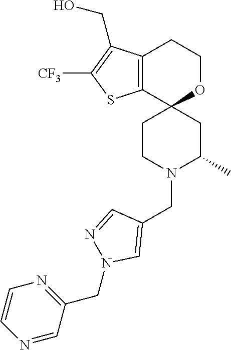 [SMILES: C[C@H]1C[C@@]2(CCN1Cc1cnn(Cc3cnccn3)c1)OCCc1c2sc(C(F)(F)F)c1CO] | 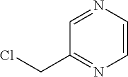 | S74³ | 1H NMR (300 MHz, MeOD) δ 8.61 (s, 1H), 8.58 (d, J = 2.6 Hz, 1H), 8.52 (s, 1H), 8.16 (s, 1H), 7.76 (s, 1H), 5.60 (s, 2H), 4.64-4.55 (m, 3H), 4.30 (d, J = 14.1 Hz, 1H), 3.99 (p, J = 6.0, 5.4 Hz, 2H), 3.57 (s, 1H), 3.38 (d, J = 4.9 Hz, 1H), 2.77 (t, J = 5.5 Hz, 2H), 2.51-2.25 (m, 2H), 2.24-1.95 (m, 2H), 1.70-1.60 (m, 1H), 1.57 (d, J = 6.4 Hz, 3H); LCMS m/z 494.09 [M + H]⁺ |
| 903 | 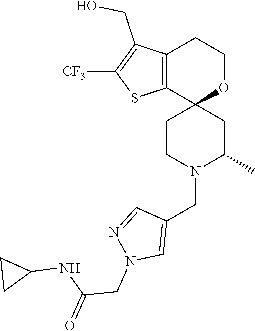 [SMILES: C[C@H]1C[C@@]2(CCN1Cc1cnn(CC(=O)NC3CC3)c1)OCCc1c2sc(C(F)(F)F)c1CO] | 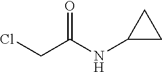 | S74³ | 1H NMR (400 MHz, DMSO) δ 10.60 (d, J = 18.6 Hz, 1H), 8.21 (t, J = 3.5 Hz, 1H), 7.76 (s, 1H), 7.46 (s, 1H), 4.58 (s, 2H), 4.27 (s, 2H), 4.21 (d, J = 13.9 Hz, 1H), 4.02 (dd, J = 14.2, 5.4 Hz, 1H), 3.72 (dq, J = 10.5, 6.0 Hz, 2H), 3.04 (d, J = 12.8 Hz, 2H), 2.84 (d, J = 10.3 Hz, 1H), 2.47 (dt, J = 15.0, 4.5 Hz, 3H), 2.05 (q, J = 14.1, 12.6 Hz, 4H), 1.28 (d, J = 6.3 Hz, 3H), 0.45 (t, J = 6.3 Hz, 2H), 0.31-0.19 (m, 2H); LCMS m/z 499.19 [M + H]⁺ |
| 904 | 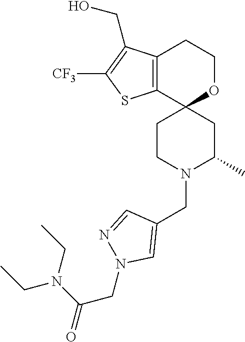 [SMILES: CCN(CC)C(=O)Cn1cc(CN2CC[C@]3(C[C@@H]2C)OCCc2c3sc(C(F)(F)F)c2CO)cn1] | 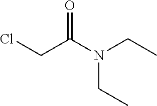 | S74³ | 1H NMR (300 MHz, MeOD) δ 7.93 (s, 1H), 7.71 (s, 1H), 5.20 (s, 2H), 4.71-4.53 (m, 3H), 4.24 (d, J = 14.0 Hz, 1H), 4.00 (q, J = 5.5 Hz, 2H), 3.74- 3.56 (m, 1H), 3.44 (dt, J = 19.9, 7.2 Hz, 6H), 2.77 (t, J = 5.5 Hz, 2H), 2.54- 2.30 (m, 2H), 2.17-1.93 (m, 2H), 1.56 (d, J = 6.3 Hz, 3H), 1.29 (t, J = 7.1 Hz, 3H), 1.14 (t, J = 7.1 Hz, 3H); LCMS m/z 515.11 [M + H]⁺ |
| 905 | 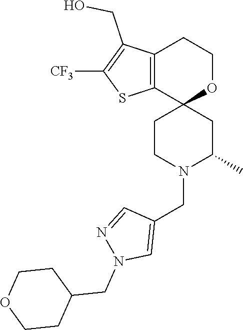 [SMILES: C[C@H]1C[C@@]2(CCN1Cc1cnn(CC3CCOCC3)c1)OCCc1c2sc(C(F)(F)F)c1CO] | 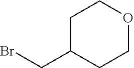 | S74³ | 1H NMR (400 MHz, DMSO) δ 10.66 (s, 1H), 7.78 (s, 1H), 7.47 (s, 1H), 4.26 (s, 2H), 4.17 (d, J = 13.9 Hz, 1H), 4.02 (dd, J = 14.2, 5.0 Hz, 1H), 3.85 (d, J = 7.1 Hz, 2H), 3.66 (td, J = 13.0, 11.2, 5.1 Hz, 4H), 3.06 (t, J = 11.1 Hz, 4H), 2.82 (q, J = 4.7, 4.2 Hz, 1H), 2.48 (t, J = 5.5 Hz, 2H), 2.04 (q, J = 14.0, 13.3 Hz, 4H), 1.86 (ddd, J = 11.4, 7.5, 4.2 Hz, 1H), 1.27 (d, J = 6.4 Hz, 3H), 1.21-1.12 (m, 2H), 1.05 (tt, J = 12.1, 6.0 Hz, 2H); LCMS m/z 500.23 [M + H]⁺ |
| 906 | 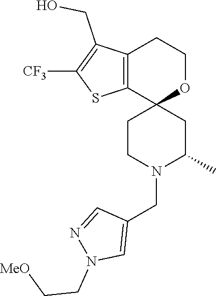 [SMILES: COCCn1cc(CN2CC[C@]3(C[C@@H]2C)OCCc2c3sc(C(F)(F)F)c2CO)cn1] | 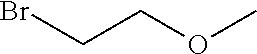 | S74³ | 1H NMR (300 MHz, MeOD) δ 7.96 (s, 1H), 7.72 (s, 1H), 4.64-4.51 (m, 3H), 4.36 (t, J = 5.0 Hz, 2H), 4.27 (d, J = 14.1 Hz, 1H), 4.09-3.87 (m, 2H), 3.75 (t, J = 5.1 Hz, 2H), 3.66-3.49 (m, 1H), 3.37 (d, J = 4.9 Hz, 2H), 3.30 (s, 3H), 2.77 (t, J = 5.5 Hz, 2H), 2.36 (dd, J = 19.0, 16.0 Hz, 2H), 2.06 (q, J = 13.4 Hz, 2H), 1.56 (d, J = 6.4 Hz, 3H); LCMS m/z 460.16 [M + H]⁺ |
| 907 | 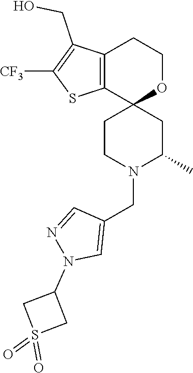 [SMILES: C[C@H]1C[C@@]2(CCN1Cc1cnn(C3CS(=O)(=O)C3)c1)OCCc1c2sc(C(F)(F)F)c1CO] | 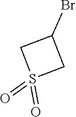 | S74³ | 1H NMR (400 MHz, DMSO) δ 10.81 (s, 1H), 8.08 (s, 1H), 7.74 (s, 1H), 5.34 (td, J = 9.2, 4.6 Hz, 1H), 4.83-4.65 (m, 2H), 4.57 (dd, J = 15.1, 5.3 Hz, 2H), 4.35 (d, J = 19.2 Hz, 3H), 4.10 (dd, J = 14.0, 5.7 Hz, 1H), 3.82 (dp, J = 17.1, 5.7 Hz, 2H), 3.14 (d, J = 12.5 Hz, 2H), 2.99- 2.92 (m, 1H), 2.60 (q, J = 7.0, 5.4 Hz, 2H), 2.25- 1.93 (m, 4H), 1.38 (d, J = 6.3 Hz, 3H); LCMS m/z 506.12 [M + H]⁺ |
| 908 | 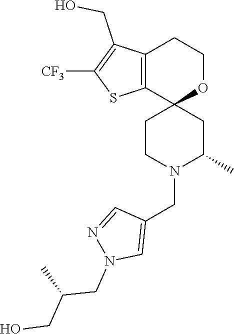 [SMILES: C[C@@H](CO)Cn1cc(CN2CC[C@]3(C[C@@H]2C)OCCc2c3sc(C(F)(F)F)c2CO)cn1] | 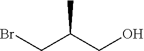 | S74³ | 1H NMR (400 MHz, DMSO) δ 10.50 (s, 1H), 7.95 (s, 1H), 7.65 (s, 1H), 4.46 (s, 2H), 4.38 (d, J = 13.9 Hz, 1H), 4.20 (ddd, J = 19.4, 13.5, 5.5 Hz, 2H), 3.98-3.82 (m, 3H), 3.25 (dd, J = 15.9, 8.7 Hz, 4H), 3.02 (s, 1H), 2.68 (t, J = 5.5 Hz, 2H), 2.31-2.01 (m, 6H), 1.46 (d, J = 6.3 Hz, 3H), 0.77 (d, J = 6.7 Hz, 3H); LCMS m/z 474.27 [M + H]⁺ |
| 909 | 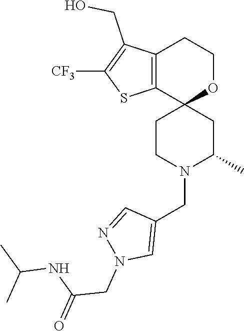 [SMILES: CC(C)NC(=O)Cn1cc(CN2CC[C@]3(C[C@@H]2C)OCCc2c3sc(C(F)(F)F)c2CO)cn1] | 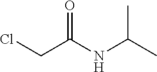 | S74³ | 1H NMR (400 MHz, DMSO) δ 9.76 (s, 1H), 7.28-7.06 (m, 1H), 6.96 (s, 1H), 6.65 (s, 1H), 3.79 (s, 2H), 3.41 (d, J = 13.8 Hz, 1H), 3.22 (dd, J = 14.0, 5.3 Hz, 1H), 2.98- 2.87 (m, 2H), 2.86-2.80 (m, 1H), 2.24 (d, J = 12.7 Hz, 3H), 2.13-2.01 (m, 2H), 1.69 (t, J = 5.5 Hz, 2H), 1.33-1.10 (m, 4H), 0.48 (d, J = 6.3 Hz, 3H), 0.09 (d, J = 6.5 Hz, 6H); LCMS m/z 501.22 [M + H]⁺ |
| 910 | 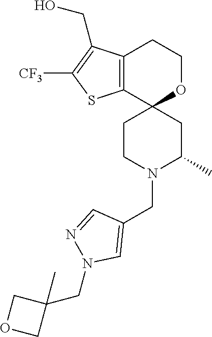 [SMILES: C[C@H]1C[C@@]2(CCN1Cc1cnn(CC3(C)COC3)c1)OCCc1c2sc(C(F)(F)F)c1CO] | 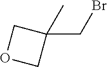 | S74³ | 1H NMR (400 MHz, DMSO) δ 12.02 (s, 1H), 8.65 (s, 2H), 4.64 (d, J = 13.9 Hz, 1H), 4.55 (d, J = 12.0 Hz, 2H), 4.46 (s, 2H), 4.31 (dd, J = 12.2, 2.3 Hz, 2H), 4.28-4.20 (m, 1H), 3.95 (dq, J = 11.3, 6.1 Hz, 2H), 3.22 (s, 3H), 3.11 (s, 2H), 2.70 (d, J = 5.7 Hz, 2H), 2.34 (dd, J = 14.4, 9.4 Hz, 3H), 2.20 (d, J = 14.6 Hz, 1H), 1.51 (d, J = 6.3 Hz, 3H), 1.27 (s, 3H); LCMS m/z 486.19 [M + H]⁺ |

Footnotes:
¹The reaction was performed at room temperature for 22 h.
²The reaction was heated at 80° C. for 72 h.
³Purification by reversed-phase HPLC. Method: Waters XSelect CSH C18 OBD Prep Column; 30 × 150 mm, 5 micron. Gradient: Acetonitrile in Water with 5 mM Hydrochloric Acid.

### Compound 911

### [(2′S, 7R)-1′-[[1-(1,1-dioxothiolan-3-yl)pyrazol-4-yl]methyl]-2′-methyl-2-(trifluoromethyl)spiro[4,5-dihydrothieno[2,3-c]pyran-7,4′-piperidine]-3-yl]methanol (911)

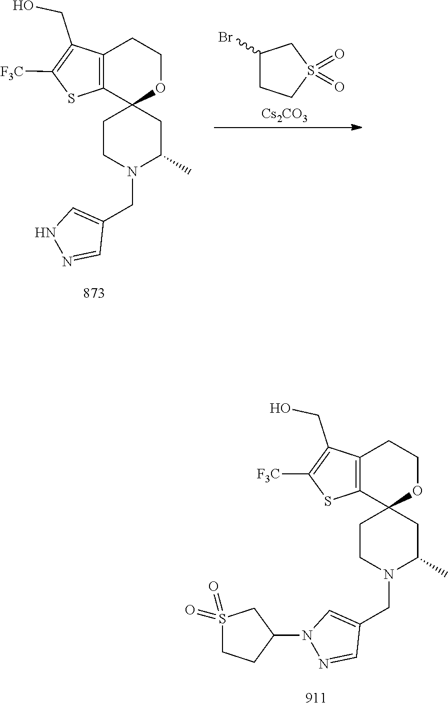

Standard Method E: N-Alkylation with Alkyl Halides

To a 2-dram vial with 3-bromotetrahydrothiophene 1,1-dioxide (22 mg, 0.11 mmol) was added cesium carbonate (64 mg, 0.20 mmol). [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (32 mg, 0.079 mmol) was added as a solution in DMF (0.4 mL). The vial was heated at 80° C. for 40 h. Purification by reversed-phase HPLC. Method: Waters XSelect CSH C18 OBD Prep Column; 30×150 mm, 5 micron. Gradient: Acetonitrile in Water with 0.2% Formic Acid. Product was isolated as [(2′S,7R)-1′-[[1-(1,1-dioxothiolan-3-yl)pyrazol-4-yl]methyl]-2′-methyl-2-(trifluoromethyl)spiro[4,5-dihydrothieno[2,3-c]pyran-7,4′-piperidine]-3-yl]methanol (17 mg, 41%). 1H NMR (400 MHz, MeOD) δ 7.85 (s, 1H), 7.61 (s, 1H), 5.26 (p, J=7.5 Hz, 1H), 4.58 (d, J=1.3 Hz, 2H), 4.09 (d, J=14.3 Hz, 1H), 3.94 (h, J=6.1 Hz, 2H), 3.82 (d, J=14.2 Hz, 1H), 3.67 (dd, J=13.8, 8.1 Hz, 1H), 3.60-3.53 (m, 1H), 3.53-3.40 (m, 1H), 3.25 (dt, J=13.6, 8.0 Hz, 1H), 2.94 (d, J=11.5 Hz, 2H), 2.88-2.46 (m, 5H), 2.15 (d, J=14.6 Hz, 2H), 1.92 (td, J=13.5, 4.4 Hz, 1H), 1.82-1.62 (m, 1H), 1.33 (d, J=6.3 Hz, 3H). LCMS m/z 520.44 [M+1]⁺.

### Compounds 912-928

Compounds 912-928 (see Table 41) were prepared in a single step from intermediate S74 or 873 using Standard Method E (Compound 911). Alkyl halides were obtained from commercial sources. Any modifications to methods are noted in Table 41 and accompanying footnotes.

TABLE 41
Structure and physicochemical data for compounds 912-928

| | | Alkyl Halide | Starting | ¹H NMR; LCMS m/z |
|---|---|---|---|---|
| Cmpd | Structure | Reagent | Material | [M + H]⁺ |
| 912 | 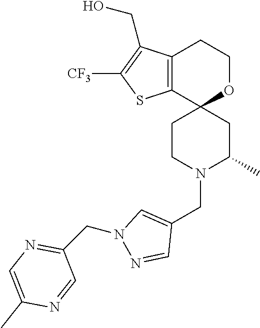 | 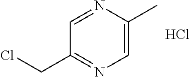 | S74¹ | LCMS m/z 508.43 [M + H]⁺ |
| 913 | 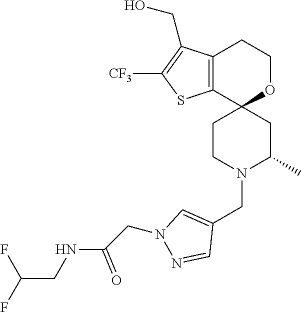 [SMILES: C[C@H]1C[C@@]2(CCN1Cc1cnn(CC(=O)NCC(F)F)c1)OCCc1c2sc(C(F)(F)F)c1CO] | 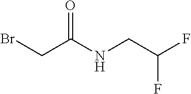 | S74 | 1H NMR (400 MHz, MeOD) δ 7.86 (s, 1H), 7.64 (s, 1H), 5.91 (tt, J = 55.8, 3.8 Hz, 1H), 4.95 (s, 2H), 4.59 (d, J = 1.5 Hz, 2H), 4.35 (d, J = 13.8 Hz, 1H), 4.12-3.81 (m, 3H), 3.61 (td, J = 15.3, 3.8 Hz, 2H), 3.29-2.94 (m, 3H), 2.75 (t, J = 5.5 Hz, 2H), 2.26 (t, J = 15.4 Hz, 2H), 2.10-1.70 (m, 2H), 1.43 (d, J = 6.3 Hz, 3H); LCMS m/z 523.42 [M + H]⁺ |
| 914 | 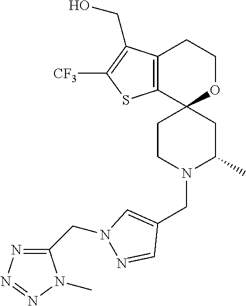 [SMILES: C[C@H]1C[C@@]2(CCN1Cc1cnn(Cc3nnnn3C)c1)OCCc1c2sc(C(F)(F)F)c1CO] | 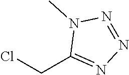 | S74 | 1H NMR (400 MHz, MeOD) δ 7.93 (s, 1H), 7.61 (s, 1H), 5.78 (s, 2H), 4.58 (s, 2H), 4.14 (s, 1H), 4.10 (s, 3H), 3.92 (dq, J = 11.9, 5.6 Hz, 3H), 2.94 (d, J = 46.3 Hz, 3H), 2.73 (t, J = 5.5 Hz, 2H), 2.16 (s, 2H), 1.92 (s, 1H), 1.76 (d, J = 13.2 Hz, 1H), 1.35 (d, J = 6.3 Hz, 3H); LCMS m/z 498.4 [M + H]⁺ |
| 915 | 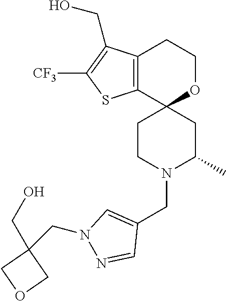 [SMILES: C[C@H]1C[C@@]2(CCN1Cc1cnn(CC3(CO)COC3)c1)OCCc1c2sc(C(F)(F)F)c1CO] | 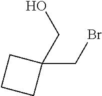 | S74 | 1H NMR (400 MHz, MeOD) δ 7.84 (s, 1H), 7.61 (s, 1H), 4.65 (d, J = 6.4 Hz, 2H), 4.61-4.54 (m, 2H), 4.50 (s, 2H), 4.43 (d, J = 6.4 Hz, 2H), 4.29 (d, J = 14.2 Hz, 1H), 4.07 (d, J = 14.1 Hz, 1H), 3.94 (hept, J = 5.9, 5.5 Hz, 2H), 3.60 (s, 2H), 3.29-2.97 (m, 3H), 2.75 (t, J = 5.5 Hz, 2H), 2.24 (t, J = 13.8 Hz, 2H), 1.99 (td, J = 13.7, 4.7 Hz, 1H), 1.87 (dd, J = 14.5, 11.9 Hz, 1H), 1.43 (d, J = 6.4 Hz, 3H); LCMS m/z 502.42 [M + H]⁺ |
| 916 | 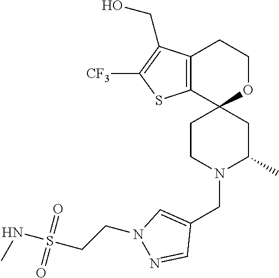 | 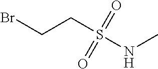 | S74 | 1H NMR (400 MHz, MeOD) δ 7.91 (s, 1H), 7.67 (s, 1H), 4.66-4.52 (m, 4H), 4.39 (d, J = 14.1 Hz, 1H), 4.14 (d, J = 14.0 Hz, 1H), 3.95 (hept, J = 5.9 Hz, 2H), 3.58 (t, J = 6.4 Hz, 2H), 3.39 (s, 1H), 3.27-3.04 (m, 2H), 2.75 (t, J = 5.5 Hz, 2H), 2.67 (s, 3H), 2.42-2.16 (m, 2H), 2.13-1.85 (m, 2H), 1.47 (d, J = 6.4 Hz, 3H); LCMS m/z 523.37 [M + H]⁺ |
| 917 | 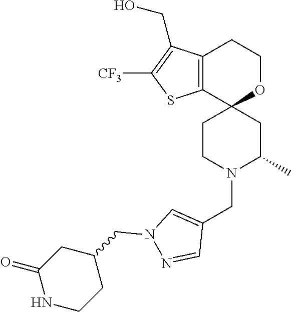 [SMILES: C[C@H]1C[C@@]2(CCN1Cc1cnn(CC3CCNC(=O)C3)c1)OCCc1c2sc(C(F)(F)F)c1CO] | 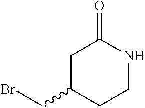 | S74 | 1H NMR (400 MHz, MeOD) δ 8.42 (s, 1H), 7.85 (s, 1H), 7.64 (s, 1H), 4.59 (s, 2H), 4.31 (d, J = 14.1 Hz, 1H), 4.23-4.12 (m, 2H), 4.09 (d, J = 14.4 Hz, 1H), 3.95 (q, J = 5.6 Hz, 2H), 3.36 (s, 2H), 3.24 (dd, J = 12.1, 4.4 Hz, 2H), 3.11 (d, J = 24.2 Hz, 2H), 2.75 (t, J = 5.5 Hz, 2H), 2.46 (s, 1H), 2.32-2.17 (m, 3H), 2.17- 1.94 (m, 2H), 1.94- 1.76 (m, 2H), 1.51 (td, J = 12.6, 12.0, 5.5 Hz, 1H), 1.44 (d, J = 6.3 Hz, 3H); LCMS m/z 513.39 [M + H]⁺ |
| 918 | 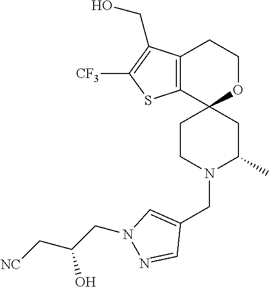 [SMILES: C[C@H]1C[C@@]2(CCN1Cc1cnn(C[C@H](O)CC#N)c1)OCCc1c2sc(C(F)(F)F)c1CO] | 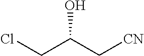 | S74 | 1H NMR (400 MHz, MeOD) δ 8.47 (s, 1H), 7.94 (s, 1H), 7.68 (s, 1H), 4.77-4.61 (m, 1H), 4.59 (s, 2H), 4.29 (d, J = 13.8 Hz, 1H), 4.11-3.74 (m, 5H), 3.25-2.83 (m, 6H), 2.74 (t, J = 5.5 Hz, 2H), 2.22 (t, J = 13.5 Hz, 2H), 1.96 (t, J = 11.9 Hz, 1H), 1.84 (t, J = 13.2 Hz, 1H), 1.41 (d, J = 6.4 Hz, 3H); LCMS m/z 485.31 [M + H]⁺ |
| 919 | 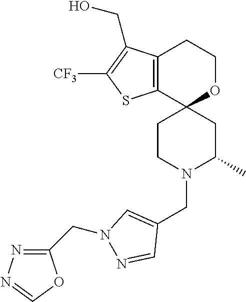 [SMILES: C[C@H]1C[C@@]2(CCN1Cc1cnn(Cc3nnco3)c1)OCCc1c2sc(C(F)(F)F)c1CO] | 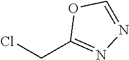 | S74 | 1H NMR (400 MHz, MeOD) δ 8.97 (s, 1H), 8.41 (s, 1H), 8.01 (s, 1H), 7.67 (s, 1H), 5.75 (s, 2H), 4.59 (s, 2H), 4.33 (d, J = 14.2 Hz, 1H), 4.03 (d, J = 14.3 Hz, 1H), 3.96 (q, J = 5.5 Hz, 2H), 3.24 (s, 1H), 3.18-2.99 (m, 2H), 2.75 (t, J = 5.5 Hz, 2H), 2.25 (t, J = 14.9 Hz, 2H), 2.04- 1.91 (m, 1H), 1.91- 1.74 (m, 1H), 1.43 (d, J = 6.3 Hz, 3H); LCMS m/z 484.32 [M + H]⁺ |
| 920 | 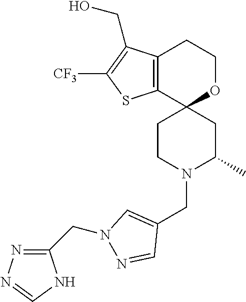 [SMILES: C[C@H]1C[C@@]2(CCN1Cc1cnn(Cc3nnc[nH]3)c1)OCCc1c2sc(C(F)(F)F)c1CO] | 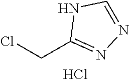 | S74¹ | 1H NMR (400 MHz, MeOD) δ 8.44 (s, 1H), 8.33 (s, 1H), 7.69 (s, 2H), 5.36 (s, 2H), 4.58 (s, 2H), 4.16-3.99 (m, 2H), 3.99- 3.84 (m, 2H), 3.04- 2.89 (m, 3H), 2.72 (t, J = 5.6 Hz, 2H), 2.11 (d, J = 14.1 Hz, 2H), 1.99-1.86 (m, 1H), 1.80-1.68 (m, 1H), 1.32 (d, J = 6.2 Hz, 3H); LCMS m/z 483.33 [M + H]⁺ |
| 921 | 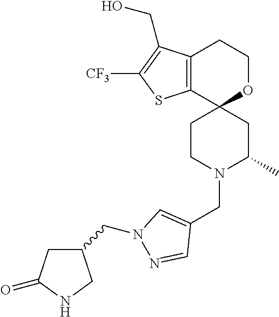 [SMILES: C[C@H]1C[C@@]2(CCN1Cc1cnn(CC3CNC(=O)C3)c1)OCCc1c2sc(C(F)(F)F)c1CO] | 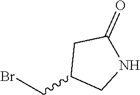 | S74 | 1H NMR (400 MHz, MeOD) δ 8.42 (s, 1H), 7.87 (s, 1H), 7.62 (s, 1H), 4.59 (s, 2H), 4.27 (d, J = 7.1 Hz, 3H), 4.06 (d, J = 14.2 Hz, 1H), 3.96 (q, J = 5.4 Hz, 2H), 3.47 (dd, J = 10.3, 7.8 Hz, 1H), 3.23 (dd, J = 10.3, 5.4 Hz, 2H), 3.05 (t, J = 8.2 Hz, 3H), 2.75 (t, J = 5.5 Hz, 2H), 2.42 (dd, J = 17.1, 8.8 Hz, 1H), 2.25 (t, J = 14.3 Hz, 2H), 2.16 (dd, J = 17.0, 6.4 Hz, 1H), 1.98 (t, J = 14.5 Hz, 1H), 1.86 (t, J = 13.2 Hz, 1H), 1.43 (d, J = 6.3 Hz, 3H); LCMS m/z 499.35 [M + H]⁺ |
| 922 | 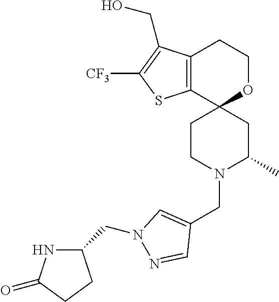 [SMILES: C[C@H]1C[C@@]2(CCN1Cc1cnn(C[C@@H]3CCC(=O)N3)c1)OCCc1c2sc(C(F)(F)F)c1CO] | 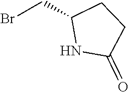 | S74 | 1H NMR (400 MHz, MeOD) δ 8.42 (s, 1H), 7.80 (s, 1H), 7.66 (s, 1H), 4.66-4.52 (m, 2H), 4.34 (d, J = 14.1 Hz, 1H), 4.29 (d, J = 4.6 Hz, 2H), 4.15- 4.05 (m, 2H), 3.96 (h, J = 6.2 Hz, 2H), 3.28-2.99 (m, 3H), 2.75 (t, J = 5.5 Hz, 2H), 2.45-2.08 (m, 4H), 2.08-1.95 (m, 2H), 1.94-1.68 (m, 2H), 1.44 (d, J = 6.4 Hz, 3H); LCMS m/z 499.35 [M + H]⁺ |
| 923 | 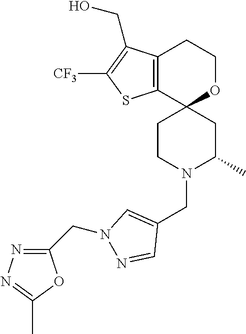 [SMILES: Cc1nnc(Cn2cc(CN3CC[C@]4(C[C@@H]3C)OCCc3c4sc(C(F)(F)F)c3CO)cn2)o1] | 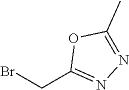 | S74 | LCMS m/z 498.32 [M + H]⁺ |
| 924 | 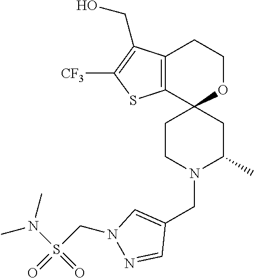 | 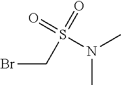 | S74 | LCMS m/z 523.33 [M + H]⁺ |
| 925 | 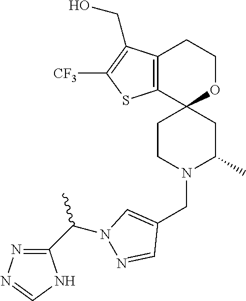 [SMILES: CC(c1nnc[nH]1)n1cc(CN2CC[C@]3(C[C@@H]2C)OCCc2c3sc(C(F)(F)F)c2CO)cn1] | 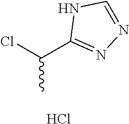 | S74^{1,2} | 1H NMR (400 MHz, MeOD) δ 8.47 (s, 1H), 8.03 (s, 1H), 7.69 (s, 1H), 5.81 (d, J = 6.9 Hz, 1H), 4.63 (s, 1H), 4.60 (s, 2H), 4.21 (d, J = 13.8 Hz, 1H), 3.99 (t, J = 5.9 Hz, 2H), 3.69-3.54 (m, 4H), 2.77 (t, J = 5.5 Hz, 2H), 2.46- 2.26 (m, 2H), 2.10-1.98 (m, 2H), 1.96 (d, J = 7.1 Hz, 3H), 1.55 (d, J = 6.4 Hz, 3H); LCMS m/z 497.37 [M + H]⁺ |
| 926 | 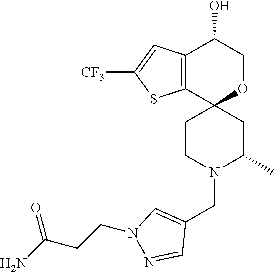 [SMILES: C[C@H]1C[C@@]2(CCN1Cc1cnn(CCC(N)=O)c1)OC[C@@H](O)c1cc(C(F)(F)F)sc12] | 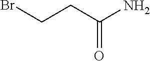 | S74 | 1H NMR (300 MHz, MeOD) δ 7.83 (s, 1H), 7.64 (s, 1H), 7.49 (d, J = 1.3 Hz, 1H), 4.56 (t, J = 3.6 Hz, 1H), 4.52-4.36 (m, 3H), 4.15 (d, J = 14.1 Hz, 1H), 3.98 (dd, J = 12.3, 3.4 Hz, 1H), 3.82 (dd, J = 12.3, 3.8 Hz, 1H), 3.41 (d, J = 21.1 Hz, 1H), 3.27-3.05 (m, 2H), 2.78 (t, J = 6.4 Hz, 2H), 2.37 (t, J = 14.2 Hz, 2H), 2.03-1.85 (m, 2H), 1.49 (d, J = 6.4 Hz, 3H); LCMS m/z 459.31 [M + H]⁺ |
| 927 | 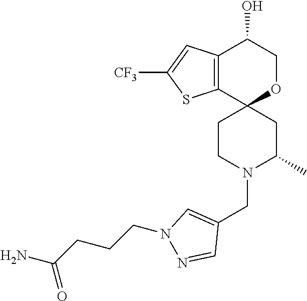 [SMILES: C[C@H]1C[C@@]2(CCN1Cc1cnn(CCCC(N)=O)c1)OC[C@@H](O)c1cc(C(F)(F)F)sc12] | 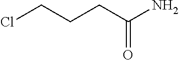 | S74³ | 1H NMR (300 MHz, MeOD) δ 7.82 (s, 1H), 7.60 (s, 1H), 7.47 (d, J = 1.4 Hz, 1H), 4.55 (t, J = 3.6 Hz, 1H), 4.35-4.15 (m, 3H), 4.04 (d, J = 14.2 Hz, 1H), 3.97 (dd, J = 12.3, 3.5 Hz, 1H), 3.79 (dd, J = 12.3, 3.9 Hz, 1H), 3.29-3.20 (m, 1H), 3.14 (d, J = 10.0 Hz, 1H), 3.00 (dd, J = 13.3, 10.7 Hz, 1H), 2.31 (t, J = 12.9 Hz, 2H), 2.17 (dq, J = 12.3, 6.5 Hz, 4H), 2.05- 1.83 (m, 2H), 1.44 (d, J = 6.4 Hz, 3H); LCMS m/z 473.21 [M + H]⁺ |
| 928 | 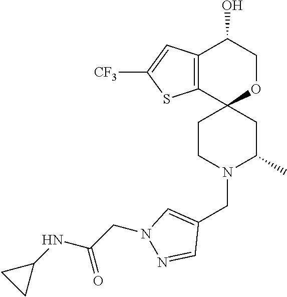 [SMILES: C[C@H]1C[C@@]2(CCN1Cc1cnn(CC(=O)NC3CC3)c1)OC[C@@H](O)c1cc(C(F)(F)F)sc12] | 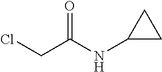 | S74⁴ | 1H NMR (300 MHz, MeOD) δ 7.87 (s, 1H), 7.65 (s, 1H), 7.49 (s, 1H), 4.84 (s, 2H), 4.57 (t, J = 3.6 Hz, 1H), 4.45 (d, J = 14.1 Hz, 1H), 4.10 (d, J = 14.2 Hz, 1H), 3.99 (dd, J = 12.3, 3.5 Hz, 1H), 3.83 (dd, J = 12.3, 3.9 Hz, 1H), 3.52-3.38 (m, 1H), 3.29-3.21 (m, 1H), 3.14 (d, J = 12.2 Hz, 1H), 2.70 (dt, J = 7.2, 3.5 Hz, 1H), 2.37 (t, J = 14.8 Hz, 2H), 1.97 (d, J = 13.8 Hz, 2H), 1.49 (d, J = 6.4 Hz, 3H), 0.74 (td, J = 7.2, 5.1 Hz, 2H), 0.59-0.45 (m, 2H); LCMS m/z 485.36 |
| | | | | [M + H]⁺ |

Footnotes:
¹3.5 eq of Cs2CO3 were used.
²Purification by reversed-phase HPLC. Method: Waters XSelect CSH C18 OBD Prep Column; 30 × 150 mm, 5 micron. Gradient: Acetonitrile in Water with 5 mM Hydrochloric Acid.
³After 26 h at 90° C., additional 4-chlorobutanamide (15.2 mg, 0.125 mmol) and cesium carbonate (60 mg, 0.18 mmol) were added and the reaction was heated at 90° C. for another 24 h.
⁴The reaction was performed at 90° C. for 4 h.

### Compound 929

### 3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propanamide (929)

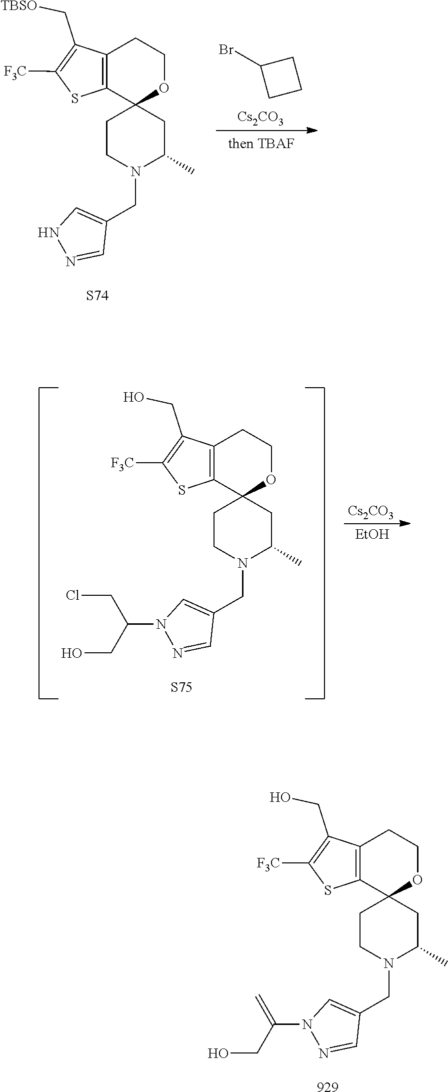

To tert-butyl-dimethyl-[[(2′S,7R)-2′-methyl-1′-(1H-pyrazol-4-ylmethyl)-2-(trifluoromethyl) spiro[4,5-dihydrothieno[2,3-c]pyran-7,4′-piperidine]-3-yl]methoxy]silane S74 (30 mg, 0.058 mmol) in a 1-dram vial was added 3-bromooxetane (16 mg, 0.12 mmol), followed by cesium carbonate (57 mg, 0.17 mmol) and DMF (200 μL). The reaction mixture was heated to 80° C. After 2 h, the reaction mixture was diluted with 0.4 mL of DMF and treated with tetrabutylammonium fluoride solution (110 μL of 1 M, 0.1100 mmol). After 90 min, the reaction was concentrated to remove excess THF and purified by reversed-phase HPLC. Method: Waters XSelect CSH C18 OBD Prep Column; 30×150 mm, 5 micron. Gradient: Acetonitrile in Water with 5 mM Hydrochloric Acid. A ring-opening product 3-chloro-2-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propan-1-ol S75 was isolated (36 mg, 0.11 mmol). The intermediate was treated with cesium carbonate (36 mg, 0.11 mmol). Then EtOH (400 μL) was added at RT. Heated to 70° C. for 5 h, and the reaction was quenched with saturated NaHCO3 solution and extracted with EtOAc (×4). The combined organic layer was dried over Na2SO4, filtered, and concentrated. The crude material was purified by silica gel chromatography (Column: 4 g column ×2, Gradient: 0-16% MeOH in DCM) afforded the unexpected product allylic alcohol rather than oxetane based on 1H NMR. The vinyl protons 1H-1H coupling can be observed on 400 MHz instrument, showing J=1.5 Hz. Product was isolated as 2-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]prop-2-en-1-ol (7.5 mg, 48%). 1H NMR (400 MHz, CDCl3) δ 7.77 (s, 1H), 7.54 (s, 1H), 5.21 (d, J=1.5 Hz, 1H), 4.94-4.86 (m, 1H), 4.70-4.60 (m, 2H), 4.56 (d, J=0.7 Hz, 2H), 4.02-3.84 (m, 3H), 3.53 (d, J=14.2 Hz, 1H), 2.86-2.59 (m, 4H), 2.53 (t, J=11.9 Hz, 1H), 2.04 (ddd, J=11.7, 9.8, 2.9 Hz, 2H), 1.90 (t, J=13.5 Hz, 1H), 1.83-1.70 (m, 1H), 1.24 (d, J=6.3 Hz, 3H). LCMS m/z 458.31 [M+H]⁺.

### Compound 930

### 4-[1-hydroxy-2-[4-[[(2′S, 7R)-3-(hydroxymethyl)-2′-methyl-2-(trifluoromethyl)spiro[4,5-dihydro-thieno[2,3-c]pyran-7,4′-piperidine]-1′-yl]methyl]pyrazol-1-yl]ethyl]tetrahydropyran-4-ol (930)

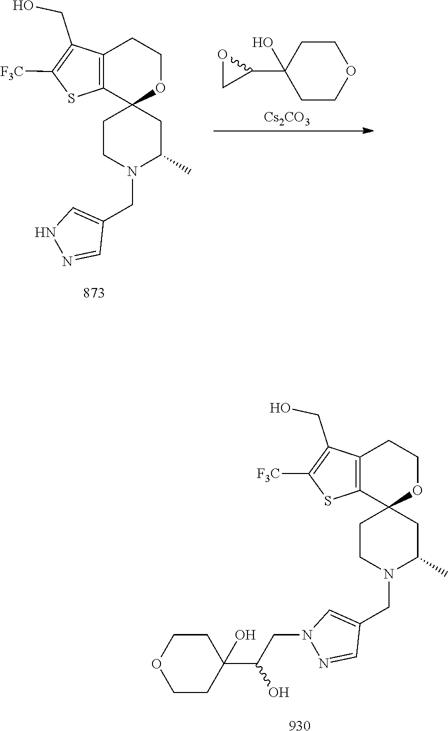

Standard Method F: N-Alkylation with Epoxides Using Cesium Carbonate as the Base

To 4-(oxiran-2-yl)tetrahydro-2H-pyran-4-ol (36 mg, 0.25 mmol) in a 1-dram vial was added cesium carbonate (171 mg, 0.53 mmol). Then [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (84 mg, 0.21 mmol) was added in 1 mL of DMF. The reaction vials were heated to 60° C. for 14 h and later heated to 90° C. for another 14 h. Purified by reversed-phase HPLC. Method: C18 Waters Sunfire column (30×150 mm, 5 micron) with 10 mM ammonium hydroxide. Product was isolated as 4-[1-hydroxy-2-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]ethyl]tetrahydropyran-4-ol (37 mg, 30%). 1H NMR (300 MHz, CDCl3) δ 7.48 (s, 1H), 7.40 (s, 1H), 4.66 (s, 2H), 4.25 (d, J=5.3 Hz, 1H), 4.08 (s, 1H), 3.87 (dtd, J=29.8, 15.6, 13.4, 8.9 Hz, 8H), 3.60-3.40 (m, 1H), 2.75 (q, J=7.5, 6.2 Hz, 3H), 2.52 (d, J=11.7 Hz, 2H), 2.01 (s, 1H), 1.77 (ddd, J=38.7, 26.9, 10.8 Hz, 7H), 1.22 (d, J=6.2 Hz, 3H). LCMS m/z 546.4 [M+1]⁺.

### Compounds 931-942

Compounds 931-942 (see Table 42) were prepared in a single step from intermediate 873 using Standard Method F. Epoxides were obtained from commercial sources. Any modifications to methods are noted in Table 42 and accompanying footnotes.

TABLE 42
Structure and physicochemical data for compounds 931-942

| | | Epoxide | Starting | ¹H NMR; LCMS m/z |
|---|---|---|---|---|
| Cmpd | Structure | Reagent | Material | [M + H]⁺ |
| 931 | 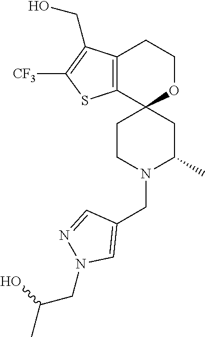 [SMILES: CC(O)Cn1cc(CN2CC[C@]3(C[C@@H]2C)OCCc2c3sc(C(F)(F)F)c2CO)cn1] | 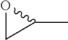 | S74¹ | 1H NMR (300 MHz, CDCl3) δ 7.46 (s, 1H), 7.39 (s, 1H), 4.64 (s, 2H), 4.29-4.07 (m, 2H), 3.93 (qd, J = 12.9, 12.1, 7.0 Hz, 4H), 3.56 (d, J = 14.8 Hz, 1H), 2.92-2.34 (m, 4H), 2.01 (d, J = 8.3 Hz, 2H), 1.91-1.81 (m, 1H), 1.81- 1.67 (m, 2H), 1.22 (d, J = 6.3 Hz, 6H); LCMS m/z 460.29 [M + H]⁺ |
| 932 | 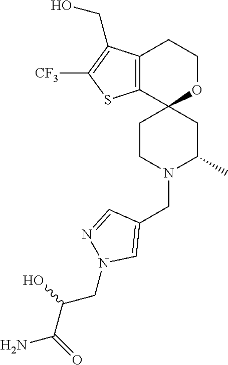 [SMILES: C[C@H]1C[C@@]2(CCN1Cc1cnn(CC(O)C(N)=O)c1)OCCc1c2sc(C(F)(F)F)c1CO] | 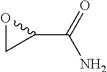 | S74¹ | 1H NMR (300 MHz, CDCl3) δ 7.46 (d, J = 3.2 Hz, 1H), 7.38 (s, 1H), 6.74 (s, 1H), 5.47 (s, 1H), 5.30 (s, 1H), 4.64 (s, 2H), 4.47 (s, 3H), 4.03-3.73 (m, 3H), 3.60 (t, J = 14.1 Hz, 1H), 2.71 (s, 3H), 2.51 (s, 2H), 1.87 (s, 1H), 1.67 (s, 1H), 1.20 (d, J = 6.2 Hz, 3H); LCMS m/z 489.27 [M + H]⁺ |
| 933 | 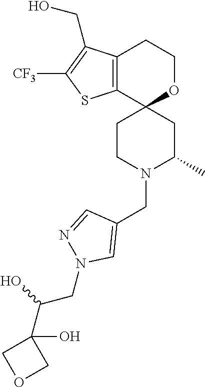 [SMILES: C[C@H]1C[C@@]2(CCN1Cc1cnn(CC(O)C3(O)COC3)c1)OCCc1c2sc(C(F)(F)F)c1CO] | 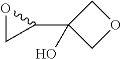 | S74 | 1H NMR (300 MHz, CDCl3) δ 7.45 (s, 1H), 7.38 (s, 1H), 4.74-4.56 (m, 3H), 4.53 (d, J = 6.9 Hz, 1H), 4.50-4.43 (m, 1H), 4.39 (dd, J = 7.2, 3.7 Hz, 1H), 4.35-4.15 (m, 3H), 4.05-3.71 (m, 5H), 3.55 (dd, J = 14.3, 3.4 Hz, 1H), 2.77-2.62 (m, 3H), 2.62-2.38 (m, 2H), 2.00 (d, J = 10.3 Hz, 2H), 1.84 (td, J = 13.3, 4.5 Hz, 1H), 1.19 (d, J = 6.2 Hz, 3H); LCMS m/z 518.36 [M + H]⁺ |
| 934 | 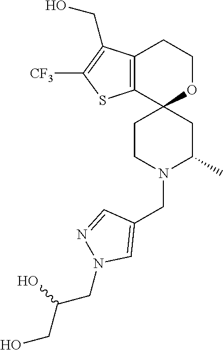 [SMILES: C[C@H]1C[C@@]2(CCN1Cc1cnn(CC(O)CO)c1)OCCc1c2sc(C(F)(F)F)c1CO] | 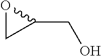 | S74 | LCMS m/z 476.33 [M + H]⁺ |
| 935 | 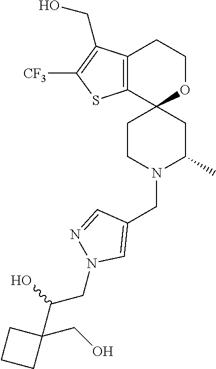 [SMILES: C[C@H]1C[C@@]2(CCN1Cc1cnn(CC(O)C3(CO)CCC3)c1)OCCc1c2sc(C(F)(F)F)c1CO] | 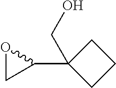 | S74 | 1H NMR (300 MHz, CDCl3) δ 7.44 (s, 1H), 7.40 (s, 1H), 4.64 (d, J = 5.4 Hz, 2H), 4.32 (s, 1H), 4.24-4.05 (m, 3H), 3.92 (d, J = 5.3 Hz, 2H), 3.82 (s, 2H), 3.62- 3.47 (m, 1H), 2.89 (s, 1H), 2.79-2.35 (m, 4H), 2.13-1.59 (m, 11H), 1.20 (d, J = 6.2 Hz, 3H); LCMS m/z 530.42 [M + H]⁺ |
| 936 | 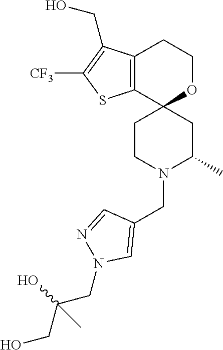 [SMILES: C[C@H]1C[C@@]2(CCN1Cc1cnn(CC(C)(O)CO)c1)OCCc1c2sc(C(F)(F)F)c1CO] | 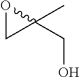 | S74 | 1H NMR (300 MHz, CDCl3) δ 7.47 (s, 1H), 7.38 (s, 1H), 4.64 (s, 2H), 4.24 (d, J = 14.0 Hz, 1H), 4.06 (d, J = 14.0 Hz, 1H), 3.96- 3.79 (m, 3H), 3.66 (s, 1H), 3.53 (dd, J = 14.3, 4.3 Hz, 1H), 3.43 (d, J = 12.0 Hz, 1H), 3.29 (d, J = 11.8 Hz, 1H), 2.89 (s, 1H), 2.70 (s, 3H), 2.61- 2.38 (m, 2H), 2.02 (t, J = 11.4 Hz, 2H), 1.84 (td, J = 13.3, 4.5 Hz, 1H), 1.68 (d, J = 12.7 Hz, 1H), 1.19 (d, J = 6.2 Hz, 3H), 1.14 (s, 3H); LCMS m/z 490.37 [M + H]⁺ |
| 937 | 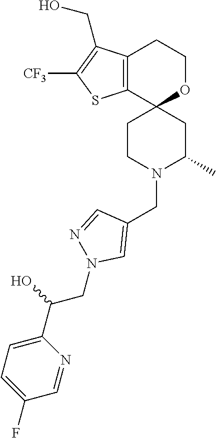 [SMILES: C[C@H]1C[C@@]2(CCN1Cc1cnn(CC(O)c3ccc(F)cn3)c1)OCCc1c2sc(C(F)(F)F)c1CO] | 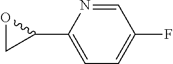 | S74 | 1H NMR (300 MHz, CDCl3) δ 8.39 (d, J = 2.6 Hz, 1H), 7.42 (s, 1H), 7.39 (s, 2H), 7.22 (d, J = 4.3 Hz, 1H), 5.14 (s, 1H), 4.94 (s, 1H), 4.64 (s, 2H), 4.51 (ddd, J = 13.8, 3.6, 2.2 Hz, 1H), 4.39 (ddd, J = 13.8, 6.5, 4.5 Hz, 1H), 3.91 (hept, J = 6.0 Hz, 2H), 3.76 (d, J = 2.3 Hz, 1H), 3.49 (dd, J = 14.3, 3.9 Hz, 1H), 2.72 (dt, J = 6.6, 3.3 Hz, 2H), 2.66- 2.27 (m, 3H), 2.00 (t, J = 10.3 Hz, 2H), 1.82 (td, J = 13.2, 4.4 Hz, 1H), 1.74-1.64 (m, 1H), 1.16 (dd, J = 6.2, 3.1 Hz, 3H); LCMS m/z 541.39 [M + H]⁺ |
| 938 | 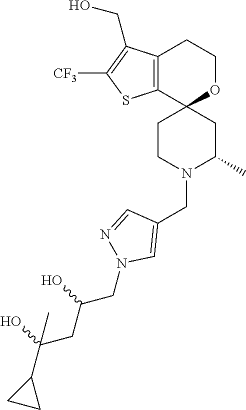 [SMILES: C[C@H]1C[C@@]2(CCN1Cc1cnn(CC(O)CC(C)(O)C3CC3)c1)OCCc1c2sc(C(F)(F)F)c1CO] | 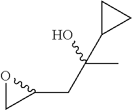 | S74 | LCMS m/z [M + H]⁺ |
| 939 | 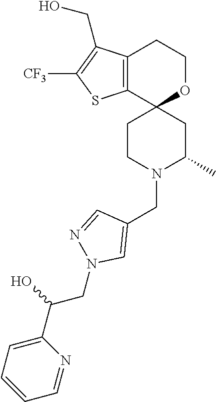 [SMILES: C[C@H]1C[C@@]2(CCN1Cc1cnn(CC(O)c3ccccn3)c1)OCCc1c2sc(C(F)(F)F)c1CO] | 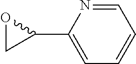 | S74 | 1H NMR (300 MHz, CDCl3) δ 8.54 (dt, J = 4.8, 1.4 Hz, 1H), 7.66 (td, J = 7.7, 1.7 Hz, 1H), 7.42 (s, 1H), 7.29 (d, J = 7.9 Hz, 1H), 7.25-7.12 (m, 2H), 5.14 (s, 1H), 5.01 (s, 1H), 4.64 (s, 2H), 4.51 (ddd, J = 13.8, 3.7, 1.9 Hz, 1H), 4.38 (ddd, J = 13.9, 6.8, 2.1 Hz, 1H), 3.90 (hept, J = 5.9 Hz, 2H), 3.79 (d, J = 14.2 Hz, 1H), 3.47 (dd, J = 14.2, 3.7 Hz, 1H), 2.72 (dt, J = 6.2, 2.9 Hz, 2H), 2.65-2.31 (m, 3H), 2.00 (ddd, J = 10.7, 5.5, 2.8 Hz, 2H), 1.91- 1.44 (m, 3H), 1.15 (dd, J = 6.2, 1.8 Hz, 3H); LCMS m/z 523.37 [M + H]⁺ |
| 940 | 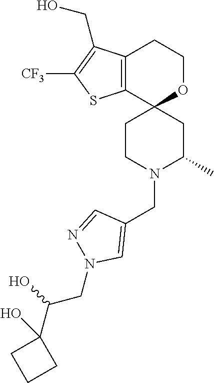 [SMILES: C[C@H]1C[C@@]2(CCN1Cc1cnn(CC(O)C3(O)CCC3)c1)OCCc1c2sc(C(F)(F)F)c1CO] | 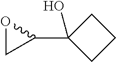 | S74 | 1H NMR (300 MHz, CDCl3) δ 7.75 (d, J = 4.9 Hz, 1H), 7.52 (d, J = 3.3 Hz, 1H), 4.64 (s, 2H), 4.48 (t, J = 13.6 Hz, 1H), 4.34 (ddd, J = 14.1, 7.7, 2.9 Hz, 1H), 4.21 (dt, J = 14.1, 9.1 Hz, 1H), 4.01 (td, J = 8.9, 8.5, 2.8 Hz, 1H), 3.95-3.83 (m, 2H), 3.77 (t, J = 14.7 Hz, 1H), 3.32 (d, J = 11.6 Hz, 2H), 2.74 (t, J = 5.5 Hz, 5H), 2.40-2.18 (m, 3H), 2.07 (d, J = 37.7 Hz, 5H), 1.86 (d, J = 5.9 Hz, 1H), 1.64 (t, J = 8.5 Hz, 1H), 1.53 (d, J = 6.3 Hz, 3H); LCMS m/z 516.42 [M + H]⁺ |
| 941 | 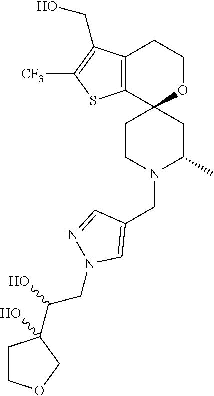 [SMILES: C[C@H]1C[C@@]2(CCN1Cc1cnn(CC(O)C3(O)CCOC3)c1)OCCc1c2sc(C(F)(F)F)c1CO] | 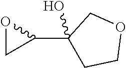 | S74 | 1H NMR (300 MHz, DMSO) δ 7.59 (s, 1H), 7.33 (s, 1H), 5.40-4.60 (m, 2H), 4.43 (s, 2H), 4.29 (ddd, J = 13.8, 6.3, 2.6 Hz, 1H), 3.95 (ddd, J = 13.3, 9.0, 3.5 Hz, 1H), 3.88-3.58 (m, 9H), 2.62 (q, J = 5.7 Hz, 4H), 2.36 (d, J = 11.7 Hz, 2H), 1.93 (t, J = 16.4 Hz, 3H), 1.79- 1.58 (m, 2H), 1.49 (t, J = 12.4 Hz, 1H), 1.11 (d, J = 6.1 Hz, 3H); LCMS m/z 532.4 [M + H]⁺ |
| 942 | 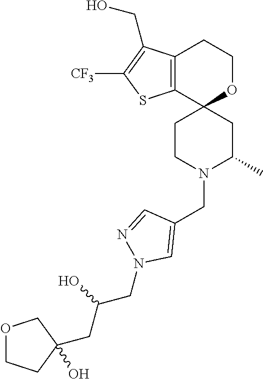 [SMILES: C[C@H]1C[C@@]2(CCN1Cc1cnn(CC(O)CC3(O)CCOC3)c1)OCCc1c2sc(C(F)(F)F)c1CO] | 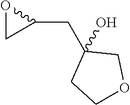 | S74 | 1H NMR (300 MHz, DMSO) δ 7.58 (d, J = 2.0 Hz, 1H), 7.34 (s, 1H), 4.43 (s, 2H), 4.24- 3.93 (m, 3H), 3.93- 3.62 (m, 6H), 3.61- 3.42 (m, 6H), 2.63 (t, J = 5.7 Hz, 3H), 2.39 (t, J = 11.9 Hz, 1H), 2.11- 1.91 (m, 2H), 1.91- 1.39 (m, 6H), 1.12 (d, J = 6.1 Hz, 3H); LCMS m/z 546.44 [M + H]⁺ |

Footnotes:
¹The reaction was performed at 80° C. for 18 h.

### Compound 943

### 4-[[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]methyl]tetrahydropyran-4-ol (943)

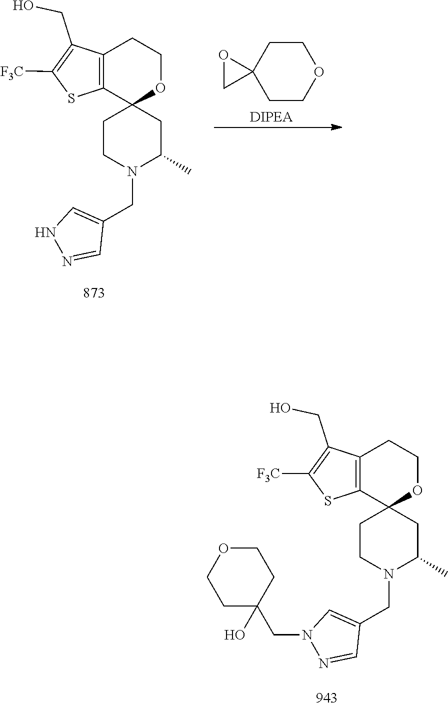

Standard Method G: N-Alkylation with Epoxides Using DIPEA as the Base

To a 1-dram vial was added [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (63 mg, 0.16 mmol), 1,6-dioxaspiro[2.5]octane (28 mg, 0.25 mmol), N-ethyl-N-isopropyl-propan-2-amine (135 μL, 0.78 mmol) and EtOH (1 mL). The reaction vial was heated to 100° C. for 1 h and later heated to 90° C. for another 105 h. Purification by reversed-phase HPLC. Method: Waters XSelect CSH C18 OBD Prep Column; 30×150 mm, 5 micron. Gradient: Acetonitrile in Water with 0.2% Formic Acid. Product was isolated as 4-[[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]methyl]tetrahydropyran-4-ol (50 mg, 52%). 1H NMR (300 MHz, MeOD) δ 8.30 (d, J=2.7 Hz, 2H), 7.91 (s, 1H), 7.67 (s, 1H), 4.72-4.56 (m, 2H), 4.52 (d, J=14.1 Hz, 1H), 4.25 (d, J=14.1 Hz, 1H), 4.19 (s, 2H), 4.05-3.86 (m, 2H), 3.86-3.67 (m, 4H), 3.53 (s, 1H), 3.42-3.33 (m, 1H), 3.30-3.15 (m, 1H), 2.77 (t, J=5.5 Hz, 2H), 2.33 (dd, J=18.9, 15.8 Hz, 2H), 2.16-1.87 (m, 2H), 1.71 (dt, =15.6, 7.8 Hz, 2H), 1.53 (d, J=6.4 Hz, 3H), 1.47-1.30 (m, 2H). LCMS m-z 516.42 [M+1]⁺.

### Compounds 944-980

Compounds 944-980 (see Table 43) were prepared in a single step from intermediate 873 using Standard Method G. Epoxides were obtained from commercial sources. Any modifications to methods are noted in Table 43 and accompanying footnotes.

TABLE 43
Structure and physicochemical data for compounds 944-980

| | | Epoxide | Starting | 1H NMR; LCMS m/z |
|---|---|---|---|---|
| Cmpd | Structure | Reagent | Material | [M + H]⁺ |
| 944 | 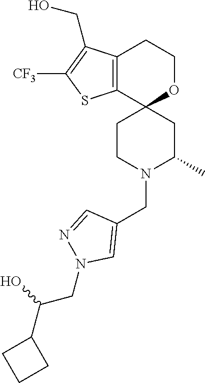 [SMILES: C[C@H]1C[C@@]2(CCN1Cc1cnn(CC(O)C3CCC3)c1)OCCc1c2sc(C(F)(F)F)c1CO] | 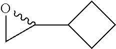 | 873 | 1H NMR (300 MHz, CDCl3) δ 7.45 (s, 1H), 7.39 (s, 1H), 4.64 (s, 2H), 4.12 (d, J = 10.9 Hz, 1H), 4.04-3.83 (m, 5H), 3.58 (dd, J = 14.2, 4.2 Hz, 1H), 2.75 (d, J = 22.7 Hz, 4H), 2.54 (d, J = 12.9 Hz, 1H), 2.30 (s, 1H), 2.09-1.77 (m, 10H), 1.26 (d, J = 6.1 Hz, 3H); LCMS m/z 500.35 [M + H]⁺ |
| 945 | 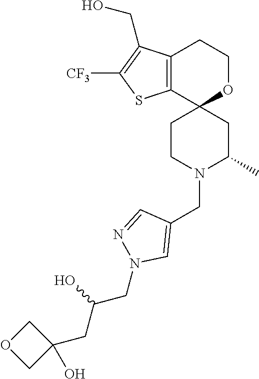 [SMILES: C[C@H]1C[C@@]2(CCN1Cc1cnn(CC(O)CC3(O)COC3)c1)OCCc1c2sc(C(F)(F)F)c1CO] | 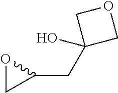 | 873 | 1H NMR (300 MHz, CDCl3) δ 8.38 (s, 1H), 7.67 (d, J = 3.2 Hz, 1H), 7.50 (s, 1H), 4.86-4.59 (m, 4H), 4.53 (dd, J = 6.5, 3.5 Hz, 1H), 4.49-4.39 (m, 2H), 4.39-4.19 (m, 2H), 4.09 (td, J = 13.0, 7.8 Hz, 1H), 3.91 (p, J = 6.3 Hz, 2H), 3.60 (dd, J = 13.8, 9.4 Hz, 1H), 3.25 (s, 2H), 2.84 (d, J = 12.3 Hz, 1H), 2.74 (t, J = 5.6 Hz, 2H), 2.13 (t, J = 17.3 Hz, 7H), 1.92 (q, J = 12.2 Hz, 1H), 1.49 (dd, J = 6.5, 2.5 Hz, 3H); LCMS m/z 532.45 [M + H]⁺ |
| 946 | 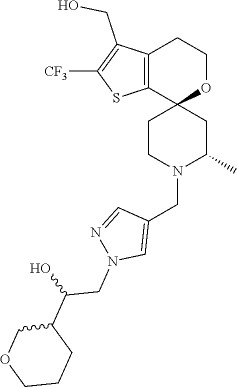 [SMILES: C[C@H]1C[C@@]2(CCN1Cc1cnn(CC(O)C3CCCOC3)c1)OCCc1c2sc(C(F)(F)F)c1CO] | 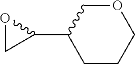 | 873 | 1H NMR (300 MHz, MeOD) δ 7.91 (s, 1H), 7.68 (s, 1H), 4.64-4.55 (m, 2H), 4.51 (d, J = 14.2 Hz, 1H), 4.40-4.21 (m, 2H), 4.14 (dd, J = 14.0, 8.0 Hz, 1H), 3.98 (q, J = 9.8, 5.5 Hz, 3H), 3.92-3.76 (m, 3H), 3.51 (dt, J = 7.4, 6.4 Hz, 2H), 3.45-3.32 (m, 4H), 2.76 (t, J = 5.5 Hz, 2H), 2.33 (dd, J = 19.3, 16.0 Hz, 2H), 2.20-1.82 (m, 3H), 1.79-1.56 (m, 3H), 1.53 (d, J = 6.4 Hz, 3H), 1.40 (dd, J = 10.3, 5.6 Hz, 1H); LCMS m/z 530.42 [M + H]⁺ |
| 947 | 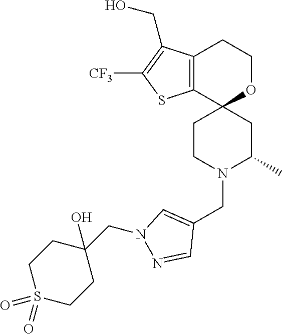 [SMILES: C[C@H]1C[C@@]2(CCN1Cc1cnn(CC3(O)CCS(=O)(=O)CC3)c1)OCCc1c2sc(C(F)(F)F)c1CO] | 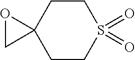 | 873 | 1H NMR (300 MHz, MeOD) δ 7.92 (s, 1H), 7.70 (s, 1H), 4.59 (d, J = 1.5 Hz, 2H), 4.52 (d, J = 14.1 Hz, 1H), 4.34-4.18 (m, 3H), 4.12-3.85 (m, 2H), 3.53 (q, J = 6.8 Hz, 1H), 3.36 (d, J = 3.6 Hz, 2H), 3.26 (d, J = 2.7 Hz, 1H), 3.04-2.83 (m, 2H), 2.76 (t, J = 5.5 Hz, 2H), 2.41-2.24 (m, 2H), 2.23-1.82 (m, 7H), 1.53 (d, J = 6.4 Hz, 3H); LCMS m/z 564.42 [M + H]⁺ |
| 948 | 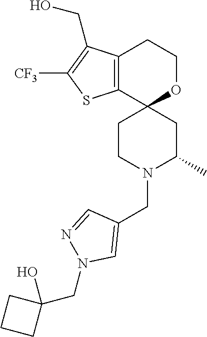 [SMILES: C[C@H]1C[C@@]2(CCN1Cc1cnn(CC3(O)CCC3)c1)OCCc1c2sc(C(F)(F)F)c1CO] | 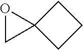 | 873 | 1H NMR (300 MHz, MeOD) δ 7.94 (s, 1H), 7.66 (s, 1H), 4.59 (d, J = 1.4 Hz, 2H), 4.52 (d, J = 14.1 Hz, 1H), 4.29 (s, 2H), 4.24 (d, J = 14.1 Hz, 1H), 3.97 (td, J = 5.7, 3.7 Hz, 2H), 3.54 (d, J = 10.9 Hz, 1H), 3.36 (dd, J = 10.2, 2.7 Hz, 1H), 3.29- 3.20 (m, 1H), 2.76 (t, J = 5.5 Hz, 2H), 2.49-2.22 (m, 2H), 2.22-2.09 (m, 2H), 2.09-1.94 (m, 4H), 1.89-1.68 (m, 1H), 1.68- 1.58 (m, 1H), 1.53 (d, J = 6.4 Hz, 3H); LCMS m/z 486.35 [M + H]⁺ |
| 949 | 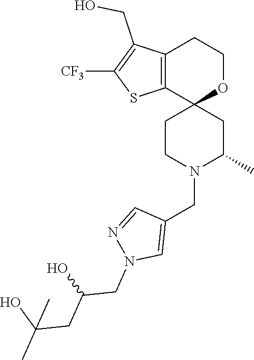 [SMILES: C[C@H]1C[C@@]2(CCN1Cc1cnn(CC(O)CC(C)(C)O)c1)OCCc1c2sc(C(F)(F)F)c1CO] | 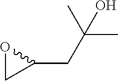 | 873 | 1H NMR (300 MHz, MeOD) δ 7.91 (s, 1H), 7.68 (s, 1H), 4.67-4.56 (m, 2H), 4.53 (d, J = 14.0 Hz, 1H), 4.38-4.17 (m, 3H), 4.09 (dd, J = 14.0, 7.7 Hz, 1H), 3.97 (t, J = 4.9 Hz, 2H), 3.55 (s, 1H), 3.43- 3.21 (m, 2H), 2.76 (t, J = 5.5 Hz, 2H), 2.34 (dd, J = 19.5, 16.1 Hz, 2H), 2.16- 1.91 (m, 2H), 1.64-1.58 (m, 2H), 1.53 (d, J = 6.4 Hz, 3H), 1.26 (d, J = 8.9 Hz, 6H); LCMS m/z 518.45 [M + H]⁺ |
| 950 | 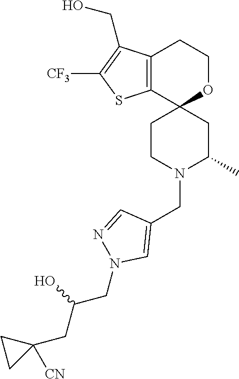 [SMILES: C[C@H]1C[C@@]2(CCN1Cc1cnn(CC(O)CC3(C#N)CC3)c1)OCCc1c2sc(C(F)(F)F)c1CO] | 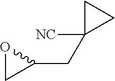 | 873 | 1H NMR (300 MHz, MeOD) δ 7.92 (s, 1H), 7.69 (s, 1H), 4.68-4.58 (m, 2H), 4.53 (d, J = 14.0 Hz, 1H), 4.41-4.08 (m, 4H), 3.98 (q, J = 5.5 Hz, 2H), 3.54 (s, 1H), 3.37 (d, J = 9.6 Hz, 1H), 2.76 (t, J = 5.5 Hz, 3H), 2.34 (t, J = 18.0 Hz, 3H), 2.17-1.84 (m, 2H), 1.72 (dd, J = 14.4, 3.2 Hz, 1H), 1.54 (dd, J = 6.9, 3.6 Hz, 4H), 1.44-1.10 (m, 3H), 1.10-0.98 (m, 1H), 0.98-0.86 (m, 1H); LCMS m/z 525.45 [M + H]⁺ |
| 951 | 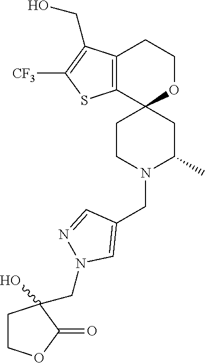 [SMILES: C[C@H]1C[C@@]2(CCN1Cc1cnn(CC3(O)CCOC3=O)c1)OCCc1c2sc(C(F)(F)F)c1CO] | 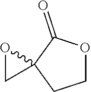 | 873 | 1H NMR (300 MHz, MeOD) δ 7.93 (d, J = 2.1 Hz, 1H), 7.69 (s, 1H), 4.59 (d, J = 1.3 Hz, 2H), 4.56- 4.44 (m, 3H), 4.42-4.16 (m, 3H), 4.08 (ddd, J = 14.4, 8.5, 6.3 Hz, 1H), 4.02- 3.87 (m, 2H), 3.52 (dt, J = 9.2, 3.5 Hz, 2H), 3.24 (d, J = 12.4 Hz, 1H), 2.76 (t, J = 5.5 Hz, 2H), 2.60-2.43 (m, 1H), 2.33 (t, J = 16.3 Hz, 2H), 2.20 (ddd, J = 13.9, 7.8, 6.3 Hz, 1H), 2.15- 1.89 (m, 2H), 1.52 (d, J = 6.4 Hz, 3H); LCMS m/z 516.38 [M + H]⁺ |
| 952 | 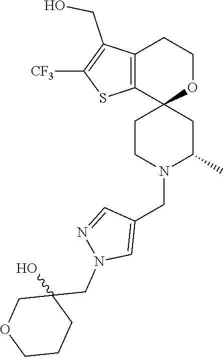 [SMILES: C[C@H]1C[C@@]2(CCN1Cc1cnn(CC3(O)CCCOC3)c1)OCCc1c2sc(C(F)(F)F)c1CO] | 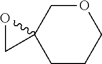 | 873 | 1H NMR (300 MHz, MeOD) δ 7.91 (s, 1H), 7.66 (s, 1H), 4.68-4.56 (m, 2H), 4.52 (d, J = 14.1 Hz, 1H), 4.37-4.14 (m, 3H), 3.97 (h, J = 6.2 Hz, 2H), 3.83-3.40 (m, 6H), 3.40- 3.32 (m, 1H), 3.24 (s, 1H), 2.77 (q, J = 6.9, 5.5 Hz, 2H), 2.33 (t, J = 16.9 Hz, 2H), 2.20-1.90 (m, 2H), 1.75 (d, J = 5.5 Hz, 3H), 1.66-1.55 (m, 2H), 1.53 (d, J = 6.4 Hz, 3H); LCMS m/z 516.42 [M + H]⁺ |
| 953 | 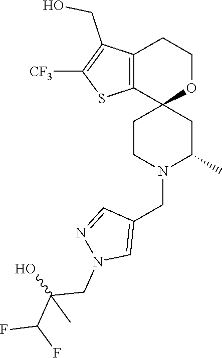 [SMILES: C[C@H]1C[C@@]2(CCN1Cc1cnn(CC(C)(O)C(F)F)c1)OCCc1c2sc(C(F)(F)F)c1CO] | 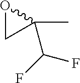 | 873 | 1H NMR (300 MHz, CDCl3) δ 7.64 (d, J = 6.3 Hz, 1H), 7.55 (d, J = 1.9 Hz, 1H), 5.74-5.19 (m, 1H), 4.67 (s, 2H), 4.48- 4.30 (m, 1H), 4.22 (t, J = 13.8 Hz, 2H), 4.08-3.82 (m, 2H), 3.73 (dd, J = 14.1, 6.3 Hz, 1H), 3.07 (s, 2H), 2.80-2.69 (m, 3H), 2.24- 2.08 (m, 6H), 1.44 (t, J = 6.3 Hz, 3H), 1.24 (d, J = 14.7 Hz, 3H); LCMS m/z 510.25 [M + H]⁺ |
| 954 | 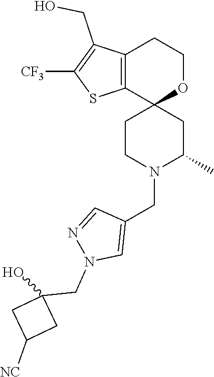 [SMILES: C[C@H]1C[C@@]2(CCN1Cc1cnn(CC3(O)CC(C#N)C3)c1)OCCc1c2sc(C(F)(F)F)c1CO] | 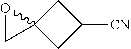 | 873 | 1H NMR (300 MHz, MeOD) δ 7.92 (s, 1H), 7.68 (d, J = 4.9 Hz, 1H), 4.59 (d, J = 1.5 Hz, 2H), 4.51 (dd, J = 14.1, 8.1 Hz, 1H), 4.31 (d, J = 22.2 Hz, 2H), 4.23 (d, J = 14.1 Hz, 1H), 3.98 (hept, J = 6.2, 5.6 Hz, 2H), 3.57-3.44 (m, 1H), 3.28- 3.19 (m, 1H), 2.87 (q, J = 8.7 Hz, 1H), 2.76 (t, J = 5.5 Hz, 2H), 2.73-2.54 (m, 3H), 2.54-2.42 (m, 1H), 2.42-2.21 (m, 5H), 2.17- 1.86 (m, 2H), 1.52 (dd, J = 6.4, 1.9 Hz, 3H); LCMS m/z 511.41 [M + H]⁺ |
| 955 | 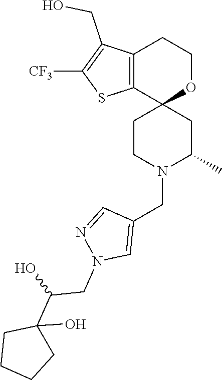 [SMILES: C[C@H]1C[C@@]2(CCN1Cc1cnn(CC(O)C3(O)CCCC3)c1)OCCc1c2sc(C(F)(F)F)c1CO] | 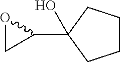 | 873 | 1H NMR (300 MHz, MeOD) δ 7.91 (s, 1H), 7.68 (s, 1H), 4.71-4.57 (m, 2H), 4.52 (d, J = 14.1 Hz, 1H), 4.49-4.40 (m, 1H), 4.24 (d, J = 14.1 Hz, 1H), 4.15 (dd, J = 13.8, 9.8 Hz, 1H), 3.98 (h, J = 6.2 Hz, 2H), 3.88-3.75 (m, 1H), 3.73-3.55 (m, 1H), 3.51- 3.42 (m, 1H), 3.34 (s, 1H), 2.76 (t, J = 5.5 Hz, 2H), 2.51-2.17 (m, 2H), 2.13- 1.91 (m, 2H), 1.91-1.57 (m, 11H), 1.53 (d, J = 6.4 Hz, 3H); LCMS m/z 530.46 [M + H]⁺ |
| 956 | 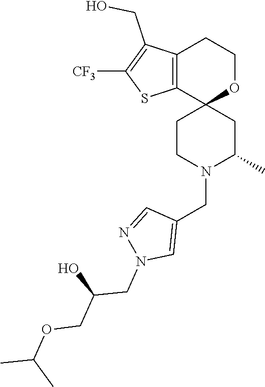 [SMILES: CC(C)OC[C@@H](O)Cn1cc(CN2CC[C@]3(C[C@@H]2C)OCCc2c3sc(C(F)(F)F)c2CO)cn1] | 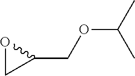 | 873 | 1H NMR (300 MHz, MeOD) δ 7.90 (s, 1H), 7.68 (s, 1H), 4.59 (d, J = 1.5 Hz, 2H), 4.52 (d, J = 14.1 Hz, 1H), 4.35 (dd, J = 13.8, 3.7 Hz, 1H), 4.29-4.11 (m, 2H), 4.07 (dt, J = 7.9, 4.4 Hz, 1H), 3.97 (td, J = 5.7, 3.6 Hz, 2H), 3.63 (h, J = 6.0 Hz, 1H), 3.55 (d, J = 8.3 Hz, 1H), 3.42 (d, J = 5.2 Hz, 2H), 3.38-3.33 (m, 1H), 3.29-3.14 (m, 1H), 2.76 (t, J = 5.5 Hz, 2H), 2.33 (dd, J = 19.7, 15.7 Hz, 2H), 2.13-1.90 (m, 2H), 1.53 (d, J = 6.4 Hz, 3H), 1.17 (dd, J = 6.1, 1.2 Hz, 6H); LCMS m/z 518.45 [M + H]⁺ |
| 957 | 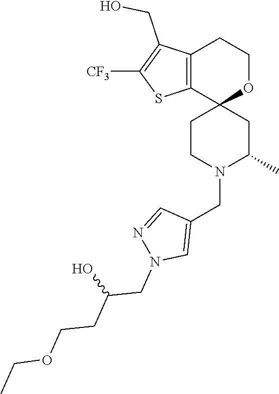 [SMILES: CCOCCC(O)Cn1cc(CN2CC[C@]3(C[C@@H]2C)OCCc2c3sc(C(F)(F)F)c2CO)cn1] | 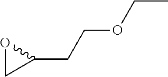 | 873 | 1H NMR (300 MHz, MeOD) δ 7.89 (s, 1H), 7.67 (s, 1H), 4.67-4.56 (m, 2H), 4.50 (d, J = 14.0 Hz, 1H), 4.35-4.06 (m, 4H), 3.97 (q, J = 5.5 Hz, 2H), 3.59 (t, J = 6.2 Hz, 3H), 3.54-3.41 (m, 4H), 2.76 (t, J = 5.4 Hz, 2H), 2.33 (t, J = 17.3 Hz, 2H), 2.15-1.87 (m, 2H), 1.86-1.69 (m, 1H), 1.61 (dd, J = 13.9, 8.0 Hz, 1H), 1.51 (d, J = 6.4 Hz, 3H), 1.18 (t, J = 7.0 Hz, 3H); LCMS m/z 518.49 [M + H]⁺ |
| 958 | 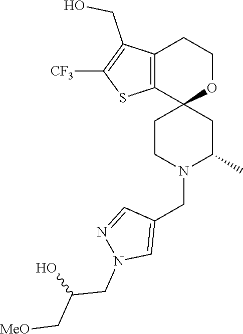 [SMILES: COCC(O)Cn1cc(CN2CC[C@]3(C[C@@H]2C)OCCc2c3sc(C(F)(F)F)c2CO)cn1] | 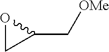 | 873 | 1H NMR (400 MHz, MeOD) δ 7.62 (s, 1H), 7.47 (s, 1H), 4.64-4.47 (m, 2H), 4.26 (dd, J = 13.6, 4.3 Hz, 1H), 4.18-3.99 (m, 2H), 3.91 (td, J = 5.7, 3.8 Hz, 2H), 3.85 (d, J = 14.1 Hz, 1H), 3.62 (dd, J = 14.2, 2.8 Hz, 1H), 3.36 (d, J = 1.2 Hz, 3H), 3.34-3.32 (m, 2H), 2.87-2.46 (m, 5H), 2.17-1.91 (m, 2H), 1.86 (td, J = 13.4, 4.5 Hz, 1H), 1.65 (dd, J = 14.1, 11.5 Hz, 1H), 1.23 (d, J = 6.2 Hz, 3H); LCMS m/z 490.41 [M + H]⁺ |
| 959 | 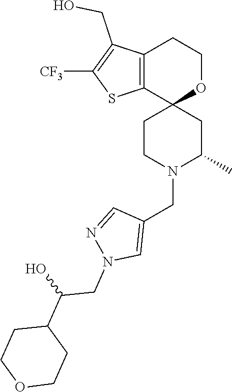 [SMILES: C[C@H]1C[C@@]2(CCN1Cc1cnn(CC(O)C3CCOCC3)c1)OCCc1c2sc(C(F)(F)F)c1CO] | 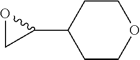 | 873 | 1H NMR (400 MHz, MeOD) δ 7.64 (d, J = 1.3 Hz, 1H), 7.47 (s, 1H), 4.57 (d, J = 1.2 Hz, 2H), 4.26 (dd, J = 14.0, 3.7 Hz, 1H), 4.08 (ddd, J = 14.0, 8.0, 2.5 Hz, 1H), 4.03-3.88 (m, 4H), 3.85 (dd, J = 14.3, 2.8 Hz, 1H), 3.71 (s, 1H), 3.62 (dd, J = 14.3, 5.4 Hz, 1H), 3.45-3.35 (m, 2H), 2.85- 2.52 (m, 5H), 2.16-1.96 (m, 2H), 1.86 (td, J = 13.4, 4.5 Hz, 1H), 1.73 (d, J = 12.6 Hz, 1H), 1.70-1.61 (m, 1H), 1.61-1.35 (m, 4H), 1.23 (d, J = 6.2 Hz, 3H); LCMS m/z 530.46 [M + H]⁺ |
| 960 | 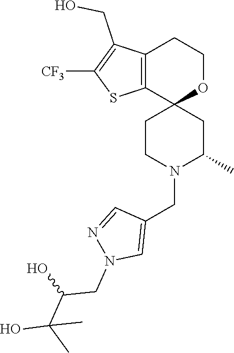 [SMILES: C[C@H]1C[C@@]2(CCN1Cc1cnn(CC(O)C(C)(C)O)c1)OCCc1c2sc(C(F)(F)F)c1CO] | 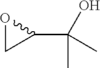 | 873 | 1H NMR (400 MHz, MeOD) δ 7.64 (s, 1H), 7.46 (s, 1H), 4.66-4.55 (m, 2H), 4.44 (dd, J = 14.0, 2.3 Hz, 1H), 4.02 (ddd, J = 14.0, 9.7, 1.6 Hz, 1H), 3.96- 3.89 (m, 2H), 3.86 (dd, J = 14.2, 4.3 Hz, 1H), 3.68 (ddd, J = 9.8, 2.3, 1.3 Hz, 1H), 3.60 (dd, J = 14.2, 8.9 Hz, 1H), 2.80-2.52 (m, 5H), 2.05 (ddt, J = 14.2, 5.8, 2.9 Hz, 2H), 1.85 (td, J = 13.5, 13.0, 3.9 Hz, 1H), 1.65 (dd, J = 14.1, 11.4 Hz, 1H), 1.28-1.17 (m, 9H); LCMS m/z 504.41 [M + H]⁺ |
| 961 | 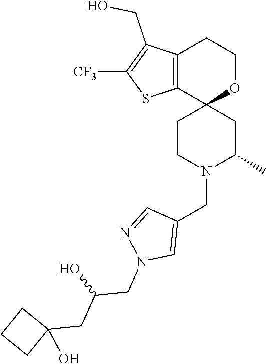 [SMILES: C[C@H]1C[C@@]2(CCN1Cc1cnn(CC(O)CC3(O)CCC3)c1)OCCc1c2sc(C(F)(F)F)c1CO] | 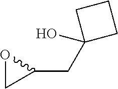 | 873 | 1H NMR (400 MHz, MeOD) δ 7.64 (s, 1H), 7.47 (d, J = 0.8 Hz, 1H), 4.57 (d, J = 1.2 Hz, 2H), 4.33-4.16 (m, 2H), 4.16-4.02 (m, 1H), 3.97-3.80 (m, 3H), 3.61 (dd, J = 14.2, 3.0 Hz, 1H), 2.80-2.48 (m, 5H), 2.05 (q, J = 7.7 Hz, 6H), 1.85 (td, J = 13.3, 4.5 Hz, 1H), 1.77-1.57 (m, 4H), 1.50 (dt, J = 11.1, 9.0 Hz, 1H), 1.23 (d, J = 6.2 Hz, 3H); LCMS m/z 530.46 [M + H]⁺ |
| 962 | 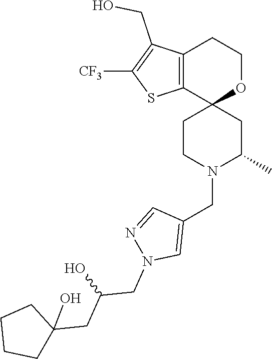 [SMILES: C[C@H]1C[C@@]2(CCN1Cc1cnn(CC(O)CC3(O)CCCC3)c1)OCCc1c2sc(C(F)(F)F)c1CO] | 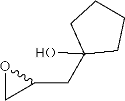 | 873 | 1H NMR (400 MHz, MeOD) δ 7.63 (s, 1H), 7.47 (d, J = 0.8 Hz, 1H), 4.57 (d, J = 1.2 Hz, 2H), 4.28 (tt, J = 8.2, 4.1 Hz, 1H), 4.18 (dd, J = 13.9, 4.3 Hz, 1H), 4.08 (ddd, J = 13.9, 7.2, 1.9 Hz, 1H), 3.99-3.74 (m, 3H), 3.60 (dd, J = 14.2, 3.2 Hz, 1H), 2.88-2.48 (m, 5H), 2.05 (ddd, J = 11.5, 6.6, 3.1 Hz, 2H), 1.96- 1.43 (m, 12H), 1.23 (d, J = 6.3 Hz, 3H); LCMS m/z 544.46 [M + H]⁺ |
| 963 | 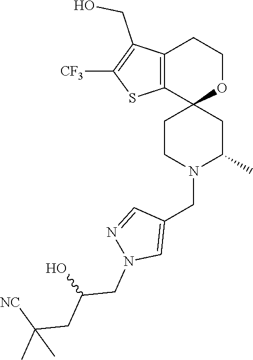 [SMILES: C[C@H]1C[C@@]2(CCN1Cc1cnn(CC(O)CC(C)(C)C#N)c1)OCCc1c2sc(C(F)(F)F)c1CO] | 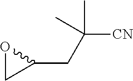 | 873 | LCMS m/z 527.44 [M + H]⁺ |
| 964 | 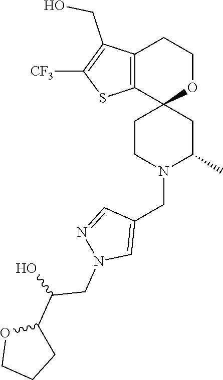 [SMILES: C[C@H]1C[C@@]2(CCN1Cc1cnn(CC(O)C3CCCO3)c1)OCCc1c2sc(C(F)(F)F)c1CO] | 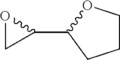 | 873 | 1H NMR (400 MHz, MeOD) δ 7.63 (s, 1H), 7.47 (d, J = 0.9 Hz, 1H), 4.57 (d, J = 1.3 Hz, 2H), 4.31-4.12 (m, 2H), 4.00-3.81 (m, 5H), 3.81-3.66 (m, 2H), 3.61 (dd, J = 14.2, 6.2 Hz, 1H), 2.82-2.47 (m, 5H), 2.05 (ddt, J = 14.4, 9.6, 2.8 Hz, 2H), 1.99-1.73 (m, 5H), 1.73-1.59 (m, 1H), 1.23 (d, J = 6.3 Hz, 3H); LCMS m/z 516.42 [M + H]⁺ |
| 965 | 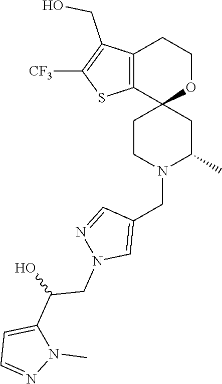 [SMILES: C[C@H]1C[C@@]2(CCN1Cc1cnn(CC(O)c3ccnn3C)c1)OCCc1c2sc(C(F)(F)F)c1CO] | 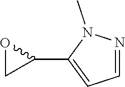 | 873 | 1H NMR (400 MHz, MeOD) δ 7.52 (d, J = 13.9 Hz, 1H), 7.48 (s, 1H), 7.39- 7.34 (m, 1H), 6.28 (t, J = 1.6 Hz, 1H), 5.34-5.01 (m, 1H), 4.58 (d, J = 1.3 Hz, 2H), 4.53-4.35 (m, 2H), 4.00-3.85 (m, 2H), 3.84-3.74 (m, 4H), 3.59 (dd, J = 14.3, 1.3 Hz, 1H), 2.79-2.33 (m, 5H), 2.04 (ddd, J = 13.9, 7.7, 2.6 Hz, 2H), 1.84 (td, J = 13.3, 4.5 Hz, 1H), 1.63 (ddd, J = 14.2, 11.5, 2.9 Hz, 1H), 1.20 (dd, J = 10.4, 6.2 Hz, 3H); LCMS m/z 526.44 M + H]⁺ |
| 966 | 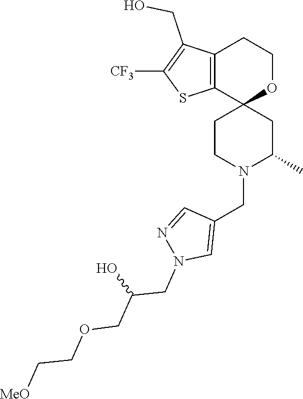 [SMILES: COCCOCC(O)Cn1cc(CN2CC[C@]3(C[C@@H]2C)OCCc2c3sc(C(F)(F)F)c2CO)cn1] | 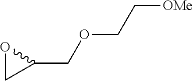 | 873 | LCMS m/z 534.43 [M + H]⁺ |
| 967 | 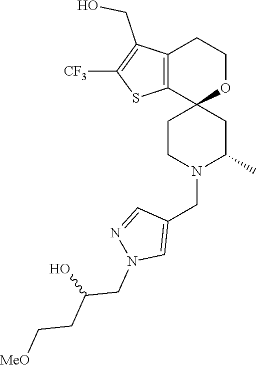 [SMILES: COCCC(O)Cn1cc(CN2CC[C@]3(C[C@@H]2C)OCCc2c3sc(C(F)(F)F)c2CO)cn1] | 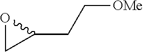 | 873 | 1H NMR (400 MHz, MeOD) δ 7.63 (s, 1H), 7.46 (s, 1H), 4.57 (d, J = 1.2 Hz, 2H), 4.21-4.12 (m, 1H), 4.08 (ddt, J = 10.0, 3.4, 1.9 Hz, 2H), 3.99-3.79 (m, 3H), 3.60 (dd, J = 14.2, 2.1 Hz, 1H), 3.57-3.43 (m, 2H), 3.32 (s, 3H), 2.83- 2.50 (m, 5H), 2.05 (ddt, J = 12.2, 5.8, 2.9 Hz, 2H), 1.85 (td, J = 13.3, 4.5 Hz, 1H), 1.77-1.50 (m, 3H), 1.23 (d, J = 6.4 Hz, 3H); LCMS m/z 504.41 [M + H]⁺ |
| 968 | 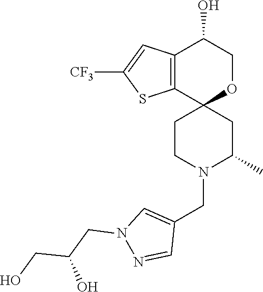 [SMILES: C[C@H]1C[C@@]2(CCN1Cc1cnn(C[C@H](O)CO)c1)OC[C@@H](O)c1cc(C(F)(F)F)sc12] | 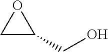 | 873 | 1H NMR (300 MHz, MeOD) δ 7.64 (s, 1H), 7.47 (s, 1H), 7.42 (s, 1H), 4.51 (t, J = 3.8 Hz, 1H), 4.28 (dd, J = 14.1, 4.4 Hz, 1H), 4.11 (dd, J = 14.0, 7.4 Hz, 1H), 4.00-3.80 (m, 3H), 3.75 (dd, J = 12.2, 4.1 Hz, 1H), 3.61 (d, J = 14.2 Hz, 1H), 3.48 (dd, J = 5.4, 1.6 Hz, 2H), 2.73 (s, 2H), 2.55 (s, 1H), 2.11 (s, 2H), 1.88- 1.60 (m, 2H), 1.25 (d, J = 6.2 Hz, 3H); LCMS m/z 462.33 [M + H]⁺ |
| 969 | 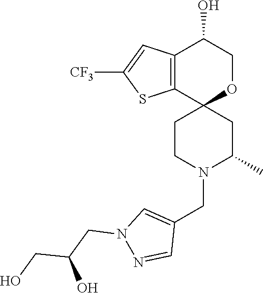 [SMILES: C[C@H]1C[C@@]2(CCN1Cc1cnn(C[C@@H](O)CO)c1)OC[C@@H](O)c1cc(C(F)(F)F)sc12] | 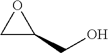 | 873 | 1H NMR (300 MHz, MeOD) δ 7.65 (s, 1H), 7.48 (s, 1H), 7.43 (s, 1H), 4.51 (t, J = 3.8 Hz, 1H), 4.29 (dd, J = 14.0, 4.3 Hz, 1H), 4.12 (dd, J = 13.9, 7.3 Hz, 1H), 4.01-3.82 (m, 3H), 3.75 (dd, J = 12.2, 4.1 Hz, 1H), 3.63 (d, J = 13.9 Hz, 1H), 3.48 (dd, J = 5.3, 1.8 Hz, 2H), 2.74 (s, 2H), 2.56 (s, 1H), 2.14 (d, J = 13.9 Hz, 2H), 1.72 (d, J = 12.3 Hz, 2H), 1.26 (d, J = 6.2 Hz, 3H); LCMS m/z 462.29 [M + H]⁺ |
| 970 | 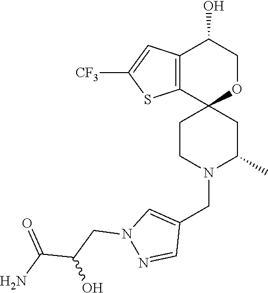 [SMILES: C[C@H]1C[C@@]2(CCN1Cc1cnn(CC(O)C(N)=O)c1)OC[C@@H](O)c1cc(C(F)(F)F)sc12] | 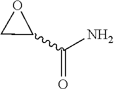 | 873 | 1H NMR (300 MHz, CDCl3) δ 7.45 (d, J = 2.1 Hz, 1H), 7.38 (s, 1H), 7.34 (d, J = 1.3 Hz, 1H), 6.79 (s, 1H), 5.57 (s, 1H), 5.30 (s, 1H), 4.63-4.36 (m, 4H), 4.00-3.75 (m, 3H), 3.64- 3.44 (m, 1H), 2.83-2.52 (m, 2H), 2.52-2.33 (m, 1H), 2.18-2.04 (m, 1H), 1.99 (dt, J = 13.8, 3.1 Hz, 1H), 1.77 (d, J = 11.8 Hz, 3H), 1.20 (dd, J = 6.2, 1.0 Hz, 3H); LCMS m/z 475.24 M + H]⁺ |
| 971 | 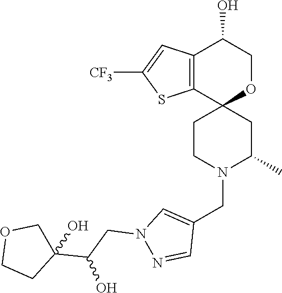 [SMILES: C[C@H]1C[C@@]2(CCN1Cc1cnn(CC(O)C3(O)CCOC3)c1)OC[C@@H](O)c1cc(C(F)(F)F)sc12] | 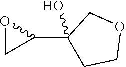 | 873¹ | 1H NMR (300 MHz, MeOD) δ 7.94 (s, 1H), 7.71 (s, 1H), 7.50 (s, 1H), 4.58 (dd, J = 8.2, 4.6 Hz, 2H), 4.42 (t, J = 13.2 Hz, 1H), 4.36-4.05 (m, 2H), 4.05- 3.50 (m, 8H), 3.47-3.24 (m, 2H), 2.44 (t, J = 16.2 Hz, 2H), 2.23-1.72 (m, 4H), 1.56 (d, J = 6.4 Hz, 3H); LCMS m/z 518.32 [M + H]⁺ |
| 972 | 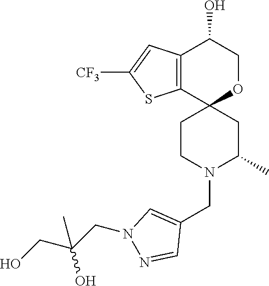 [SMILES: C[C@H]1C[C@@]2(CCN1Cc1cnn(CC(C)(O)CO)c1)OC[C@@H](O)c1cc(C(F)(F)F)sc12] | 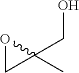 | 873¹ | 1H NMR (300 MHz, MeOD) δ 7.90 (s, 1H), 7.66 (s, 1H), 7.49 (s, 1H), 4.57 (t, J = 3.6 Hz, 1H), 4.51 (d, J = 14.1 Hz, 1H), 4.25 (d, J = 10.7 Hz, 3H), 3.98 (dd, J = 12.4, 3.5 Hz, 1H), 3.82 (dd, J = 12.4, 3.8 Hz, 1H), 3.62-3.44 (m, 1H), 3.34 (s, 2H), 3.30-3.10 (m, 2H), 2.40 (t, J = 14.9 Hz, 2H), 2.01 (t, J = 13.3 Hz, 2H), 1.53 (d, J = 6.4 Hz, 3H), 1.09 (s, 3H); LCMS m/z 476.11 [M + H]⁺ |
| 973 | 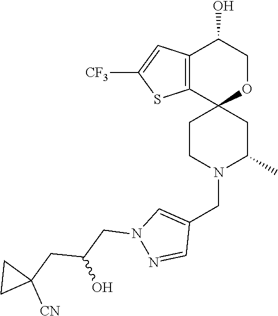 [SMILES: C[C@H]1C[C@@]2(CCN1Cc1cnn(CC(O)CC3(C#N)CC3)c1)OC[C@@H](O)c1cc(C(F)(F)F)sc12] | 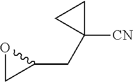 | 873¹ | 1H NMR (300 MHz, MeOD) δ 7.91 (s, 1H), 7.68 (s, 1H), 7.50 (s, 1H), 4.58 (d, J = 3.6 Hz, 1H), 4.52 (d, J = 14.1 Hz, 1H), 4.36- 4.06 (m, 4H), 3.99 (dd, J = 12.3, 3.4 Hz, 1H), 3.83 (dd, J = 12.2, 3.9 Hz, 1H), 3.68- 3.46 (m, 1H), 3.21 (d, J = 11.7 Hz, 2H), 2.40 (t, J = 15.8 Hz, 2H), 2.15-1.85 (m, 2H), 1.85-1.66 (m, 1H), 1.53 (d, J = 6.4 Hz, 4H), 1.38-1.11 (m, 2H), 1.11-0.88 (m, 2H); LCMS m/z 511.32 [M + H]⁺ |
| 974 | 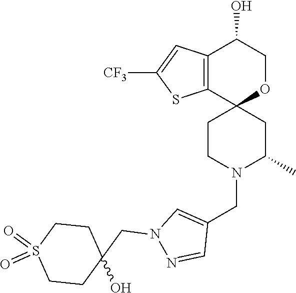 [SMILES: C[C@H]1C[C@@]2(CCN1Cc1cnn(CC3(O)CCS(=O)(=O)CC3)c1)OC[C@@H](O)c1cc(C(F)(F)F)sc12] | 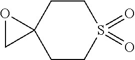 | 873¹ | 1H NMR (300 MHz, MeOD) δ 7.91 (s, 1H), 7.69 (s, 1H), 7.50 (d, J = 1.4 Hz, 1H), 4.57 (t, J = 3.6 Hz, 1H), 4.51 (d, J = 14.1 Hz, 1H), 4.32-4.17 (m, 3H), 3.98 (dd, J = 12.3, 3.4 Hz, 1H), 3.82 (dd, J = 12.3, 3.9 Hz, 1H), 3.64-3.45 (m, 1H), 3.41-3.14 (m, 4H), 2.92 (d, J = 13.8 Hz, 2H), 2.40 (t, J = 14.3 Hz, 2H), 2.15 (t, J = 13.3 Hz, 2H), 2.08-1.81 (m, 4H), 1.53 (d, J = 6.4 Hz, 3H); LCMS m/z 550.16 [M + H]⁺ |
| 975 | 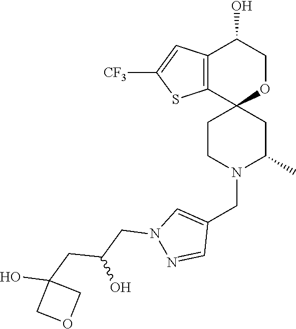 [SMILES: C[C@H]1C[C@@]2(CCN1Cc1cnn(CC(O)CC3(O)COC3)c1)OC[C@@H](O)c1cc(C(F)(F)F)sc12] | 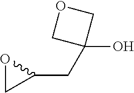 | 873¹ | 1H NMR (300 MHz, MeOD) δ 7.91 (s, 1H), 7.66 (s, 1H), 7.49 (s, 1H), 4.66- 4.45 (m, 3H), 4.38 (dd, J = 14.3, 3.3 Hz, 1H), 4.32- 4.16 (m, 2H), 3.99 (dd, J = 12.3, 3.4 Hz, 1H), 3.83 (dd, J = 10.8, 5.5 Hz, 2H), 3.67- 3.40 (m, 4H), 3.20 (d, J = 12.6 Hz, 2H), 2.40 (t, J = 15.1 Hz, 2H), 2.11-1.81 (m, 3H), 1.74 (ddd, J = 13.5, 9.9, 4.7 Hz, 1H), 1.53 (d, J = 6.4 Hz, 3H); LCMS m/z 518.32 [M + H]⁺ |
| 976 | 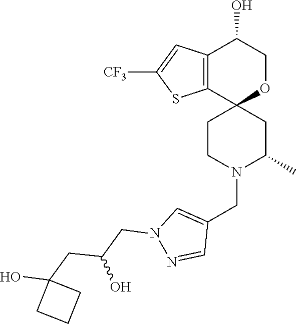 [SMILES: C[C@H]1C[C@@]2(CCN1Cc1cnn(CC(O)CC3(O)CCC3)c1)OC[C@@H](O)c1cc(C(F)(F)F)sc12] | 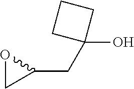 | 873¹ | 1H NMR (300 MHz, MeOD) δ 7.93 (s, 1H), 7.69 (s, 1H), 7.50 (s, 1H), 4.60 (d, J = 13.7 Hz, 2H), 4.36- 4.19 (m, 3H), 4.13 (dd, J = 12.8, 6.9 Hz, 1H), 3.99 (dd, J = 12.2, 3.3 Hz, 1H), 3.83 (dd, J = 12.3, 3.7 Hz, 1H), 3.64 (s, 1H), 3.41 (d, J = 12.1 Hz, 2H), 2.44 (t, J = 16.4 Hz, 2H), 2.09 (t, J = 8.8 Hz, 4H), 1.98 (d, J = 18.0 Hz, 2H), 1.86-1.63 (m, 3H), 1.56 (d, J = 6.4 Hz, 4H); LCMS m/z 516.33 [M + H]⁺ |
| 977 | 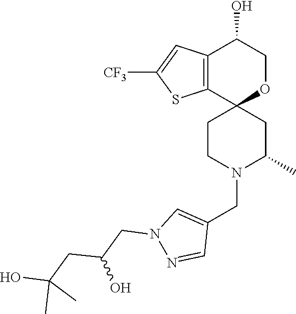 [SMILES: C[C@H]1C[C@@]2(CCN1Cc1cnn(CC(O)CC(C)(C)O)c1)OC[C@@H](O)c1cc(C(F)(F)F)sc12] | 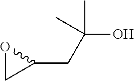 | 873¹ | 1H NMR (300 MHz, MeOD) δ 7.92 (s, 1H), 7.69 (s, 1H), 7.50 (s, 1H), 4.71- 4.52 (m, 2H), 4.40-4.20 (m, 3H), 4.10 (dd, J = 14.0, 7.8 Hz, 1H), 3.99 (dd, J = 12.3, 3.4 Hz, 1H), 3.83 (dd, J = 12.3, 3.8 Hz, 1H), 3.61 (d, J = 11.5 Hz, 1H), 3.42 (d, J = 12.3 Hz, 2H), 2.44 (t, J = 16.1 Hz, 2H), 2.11- 1.77 (m, 2H), 1.68-1.45 (m, 5H), 1.26 (d, J = 8.7 Hz, 6H); LCMS m/z 504.32 [M + H]⁺ |
| 978 | 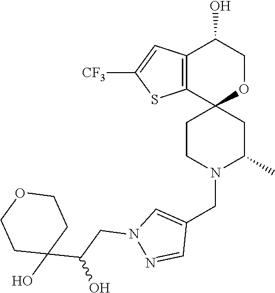 [SMILES: C[C@H]1C[C@@]2(CCN1Cc1cnn(CC(O)C3(O)CCOCC3)c1)OC[C@@H](O)c1cc(C(F)(F)F)sc12] | 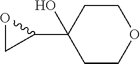 | 873¹ | 1H NMR (300 MHz, MeOD) δ 7.92 (s, 1H), 7.69 (s, 1H), 7.50 (d, J = 1.3 Hz, 1H), 4.66-4.41 (m, 3H), 4.28 (d, J = 14.1 Hz, 1H), 4.13 (dd, J = 13.9, 9.8 Hz, 1H), 3.99 (dd, J = 12.3, 3.4 Hz, 1H), 3.91-3.72 (m, 5H), 3.67 (dt, J = 9.8, 2.8 Hz, 2H), 3.38 (d, J = 12.4 Hz, 2H), 2.42 (t, J = 16.2 Hz, 2H), 2.11-1.67 (m, 4H), 1.55 (d, J = 6.3 Hz, 4H), 1.44 (d, J = 13.7 Hz, 1H); LCMS m/z 532.32 [M + H]⁺ |
| 979 | 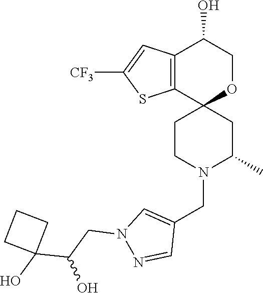 [SMILES: C[C@H]1C[C@@]2(CCN1Cc1cnn(CC(O)C3(O)CCC3)c1)OC[C@@H](O)c1cc(C(F)(F)F)sc12] | 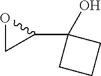 | 873¹ | 1H NMR (300 MHz, MeOD) δ 7.66 (s, 1H), 7.49 (s, 1H), 7.43 (s, 1H), 4.52 (t, J = 3.8 Hz, 1H), 4.31 (dd, J = 14.1, 3.2 Hz, 1H), 4.10 (dd, J = 14.1, 8.9 Hz, 1H), 4.02-3.81 (m, 3H), 3.80-3.61 (m, 2H), 2.76 (s, 2H), 2.59 (t, J = 12.0 Hz, 1H), 2.46-2.26 (m, 1H), 2.24-1.90 (m, 5H), 1.90-1.52 (m, 4H), 1.27 (d, J = 6.2 Hz, 3H); LCMS m/z 502.25 [M + H]⁺ |
| 980 | 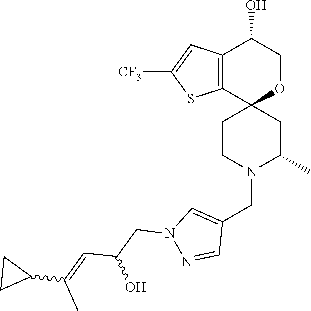 [SMILES: CC(=CC(O)Cn1cc(CN2CC[C@]3(C[C@@H]2C)OC[C@@H](O)c2cc(C(F)(F)F)sc23)cn1)C1CC1] | 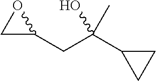 | 873^{1,2} | 1H NMR (300 MHz, MeOD) δ 8.91 (s, 1H), 8.62 (s, 1H), 7.50 (s, 1H), 4.84- 4.63 (m, 3H), 4.59 (t, J = 3.7 Hz, 2H), 4.41 (td, J = 13.9, 13.1, 7.3 Hz, 1H), 4.25 (d, J = 14.4 Hz, 1H), 4.01 (dd, J = 12.3, 3.4 Hz, 1H), 3.86 (dd, J = 12.3, 3.9 Hz, 1H), 3.65 (d, J = 25.8 Hz, 1H), 3.45-3.34 (m, 1H), 2.59-1.93 (m, 7H), 1.77 (d, J = 10.2 Hz, 1H), 1.63 (d, J = 3.0 Hz, 1H), 1.57 (d, J = 6.5 Hz, 3H), 1.42 (d, J = 3.2 Hz, 2H), 1.03-0.48 (m, 4H); LCMS m/z 512.19 [M + H]⁺ |

Footnotes:
¹The reaction was performed at 90° C. for 86 h.
²Dehydration product was isolated as the major product, which as a 1.2:1 mixture of olefin isomers.

### Compounds 981-984

Compounds 981-984 (see Table 44) were isolated as side products from the corresponding nitrile analogues, which resulted from formic acid-mediated cyclization during purification step.

TABLE 44
Structure and physicochemical data for compounds 981-984

| | | | ¹H NMR; LCMS m/z |
|---|---|---|---|
| Cmpd | Structure | Starting Material | [M + H]⁺ |
| 981 | 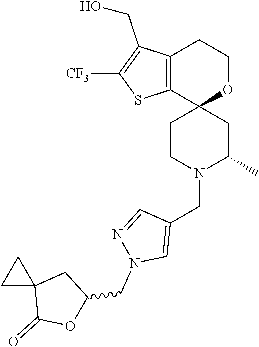 [SMILES: C[C@H]1C[C@@]2(CCN1Cc1cnn(CC3CC4(CC4)C(=O)O3)c1)OCCc1c2sc(C(F)(F)F)c1CO] | 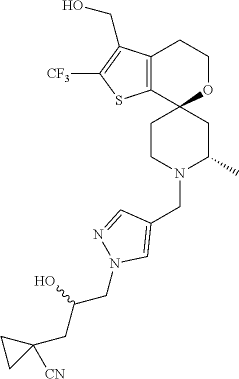 [SMILES: C[C@H]1C[C@@]2(CCN1Cc1cnn(CC(O)CC3(C#N)CC3)c1)OCCc1c2sc(C(F)(F)F)c1CO] | 1H NMR (400 MHz, MeOD) δ 7.91 (d, J = 3.1 Hz, 1H), 7.70 (s, 1H), 5.16-4.95 (m, 1H), 4.61- 4.54 (m, 3H), 4.46 (dd, J = 14.7, 5.8 Hz, 1H), 4.40 (d, J = 14.2 Hz, 1H), 4.22 (dd, J = 14.2, 2.6 Hz, 1H), 4.04-3.87 (m, 2H), 3.27-3.05 (m, 1H), 2.76 (t, J = 5.5 Hz, 2H), 2.66 (s, 3H), 2.51 (ddd, J = 13.3, 8.5, 2.0 Hz, 1H), 2.40-2.20 (m, 3H), 2.11- 1.88 (m, 2H), 1.49 (t, J = 6.8 Hz, 3H), 1.18-0.97 (m, 3H), 0.97-0.85 (m, 1H); LCMS m/z 526.27 [M + H]⁺ |
| 982 | 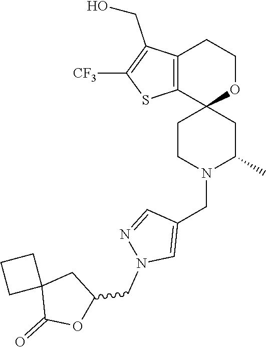 [SMILES: C[C@H]1C[C@@]2(CCN1Cc1cnn(CC3CC4(CCC4)C(=O)O3)c1)OCCc1c2sc(C(F)(F)F)c1CO] | 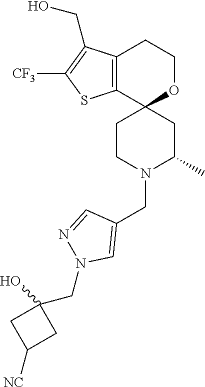 | LCMS m/z 540.31 [M + H]⁺ |
| 983 | 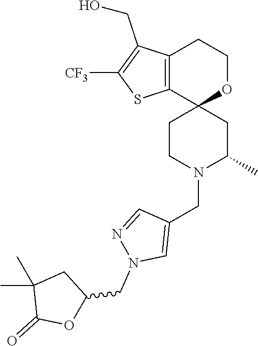 | 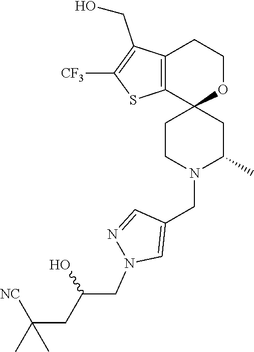 [SMILES: C[C@H]1C[C@@]2(CCN1Cc1cnn(CC(O)CC(C)(C)C#N)c1)OCCc1c2sc(C(F)(F)F)c1CO] | 1H NMR (300 MHz, CDCI3) δ 7.92 (s, 1H), 7.70 (s, 1H), 4.64-4.56 (m, 3H), 4.56-4.34 (m, 2H), 4.20 (d, J = 14.1 Hz, 1H), 3.97 (s, 2H), 3.24 (s, 3H), 2.76 (t, J = 5.5 Hz, 2H), 2.66 (s, 1H), 2.44- 2.10 (m, 3H), 2.11-1.79 (m, 3H), 1.49 (d, J = 6.4 Hz, 3H), 1.25 (s, 4H), 1.17 (d, J = 3.6 Hz, 3H); LCMS m/z 528.29 [M + H]⁺ |
| 984 | 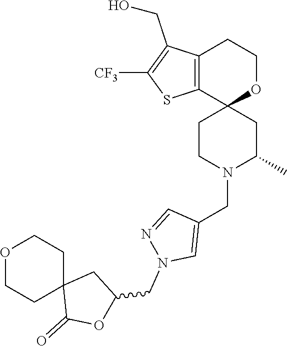 [SMILES: C[C@H]1C[C@@]2(CCN1Cc1cnn(CC3CC4(CCOCC4)C(=O)O3)c1)OCCc1c2sc(C(F)(F)F)c1CO] | 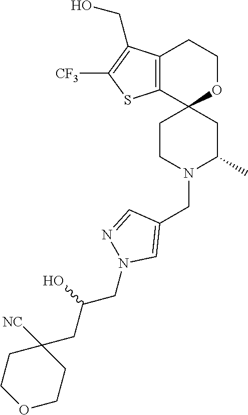 [SMILES: C[C@H]1C[C@@]2(CCN1Cc1cnn(CC(O)CC3(C#N)CCOCC3)c1)OCCc1c2sc(C(F)(F)F)c1CO] | 1H NMR (400 MHz, MeOD) δ 7.88 (s, 1H), 7.67 (s, 1H), 4.58 (d, J = 14.6 Hz, 3H), 4.41 (dd, J = 14.8, 6.5 Hz, 1H), 4.35 (d, J = 14.3 Hz, 1H), 4.13 (d, J = 14.1 Hz, 1H), 4.04- 3.81 (m, 5H), 3.71- 3.53 (m, 2H), 3.48 (d, J = 20.4 Hz, 1H), 3.19-3.05 (m, 1H), 2.75 (t, J = 5.4 Hz, 2H), 2.66 (s, 2H), 2.57 (dd, J = 13.4, 6.9 Hz, 1H), 2.27 (s, 2H), 2.08-1.76 (m, 4H), 1.64 (d, J = 13.5 Hz, 1H), 1.45 (dd, J = 6.5, 3.8 Hz, 3H), 1.34-1.22 (m, 1H); LCMS m/z 570.29 [M + H]⁺ |

### Compound 985

### (2R)-2-hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propanamide (985)

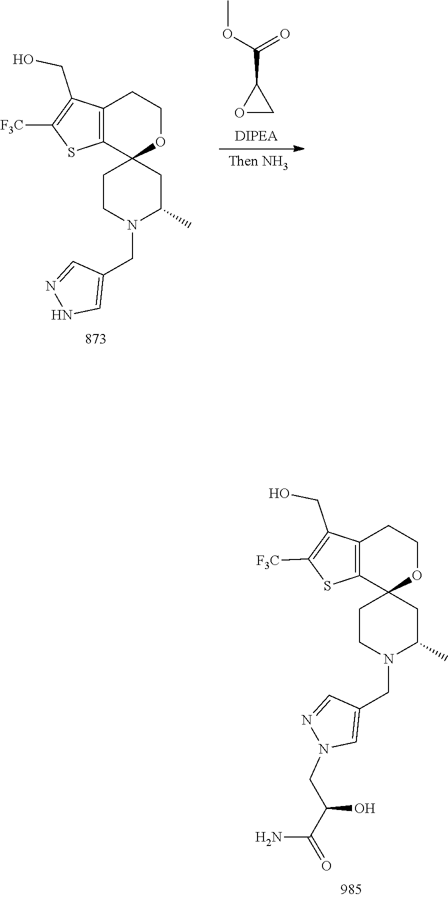

To a 2-dram vial was added [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (102 mg, 0.24 mmol), methyl (2R)-oxirane-2-carboxylate (34 μL, 0.39 mmol), EtOH (1.5 mL) and DIPEA (220 μL, 1.26 mmol). The reaction vial was heated to 90° C. for 60 h. Additional methyl (2R)-oxirane-2-carboxylate (1 eq, 21 uL) was added and the reaction vial was continued be to be heated at 90° C. for 24 h. The reaction mixture was then concentrated and dissolved in MeOH (0.3 mL). ⅓ of the solution (0.1 mL) was transferred to a 1-dram vial and treated with ammonia in MeOH (1.2 mL of 4 M, 4.8 mmol). The reaction mixture was stirred at RT for 20 h. The reaction mixture was concentrated down via rotovap. Purification by reversed-phase HPLC. Method: Waters XSelect CSH C18 OBD Prep Column; 30×150 mm, 5 micron. Gradient: Acetonitrile in Water with 0.2% Formic Acid. Product was isolated as (2R)-2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]propenamide (formic acid salt) (3.6 mg, 7%). 1H NMR (300 MHz, CD3CN) δ 8.27 (s, 3H), 7.89 (s, 1H), 7.66 (s, 1H), 4.60 (s, 2H), 4.52 (dd, J=12.7, 6.8 Hz, 2H), 4.46-4.31 (m, 2H), 4.22 (d, J=14.1 Hz, 1H), 3.98 (d, J=4.3 Hz, 2H), 3.53 (s, 1H), 2.77 (t, J=5.5 Hz, 2H), 2.34 (t, J=16.6 Hz, 2H), 1.98 (d, J=11.8 Hz, 2H), 1.51 (d, J=6.4 Hz, 3H). LCMS m/z 489.16 [M+1]⁺.

### Compound 986

### (2R)-2-hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N-methyl-propanamide (986)

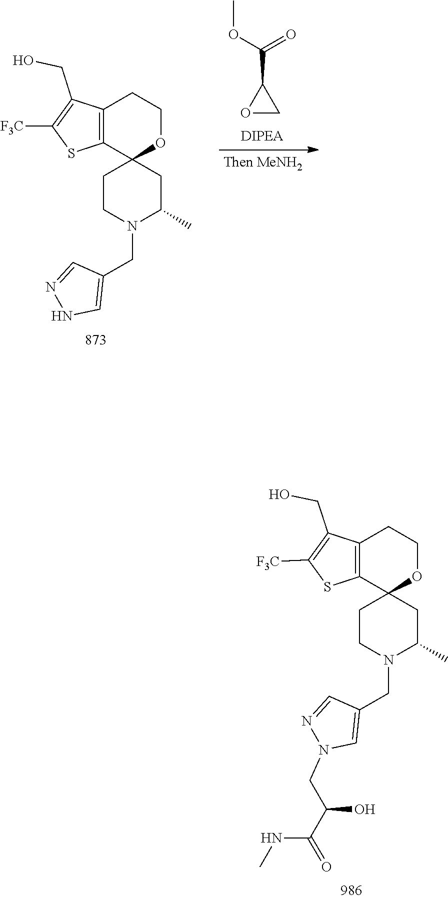

The title compound was prepared in a similar procedure as 985 with methyl amine in EtOH (600 μL of 8 M, 4.8 mmol) as the nucleophile in the second step. And the second step took 4 h. Purification by reversed-phase HPLC. Method: Waters XSelect CSH Prep Phenyl Hexyl Column; 30×150 mm, 5 micron. Gradient: Acetonitrile in Water with 0.2% Formic Acid. Subsequent purification by silica gel chromatography (Gradient: 0-12% MeOH in DCM) yielded and then SFC purification eventually provide desired product (2R)-2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N-methyl-propanamide (1.9 mg, 5%). 1H NMR (300 MHz, CDCl3) δ 7.44 (s, 1H), 7.36 (s, 1H), 6.88 (d, J=5.4 Hz, 1H), 4.64 (s, 2H), 4.59-4.27 (m, 3H), 4.04-3.75 (m, 3H), 3.53 (d, J=14.2 Hz, 1H), 2.79 (d, J=4.9 Hz, 3H), 2.69 (q, J=9.3, 7.2 Hz, 3H), 2.57 (s, 1H), 2.47 (t, J=11.8 Hz, 1H), 2.12-1.95 (m, 2H), 1.85 (td, J=13.1, 4.3 Hz, 1H), 1.76-1.66 (m, 1H), 1.19 (d, J=6.0 Hz, 3H). LCMS m/z 503.33 [M+1]⁺.

### Compound 987

### (2R)-2-hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N,N-dimethyl-propanamide (987)

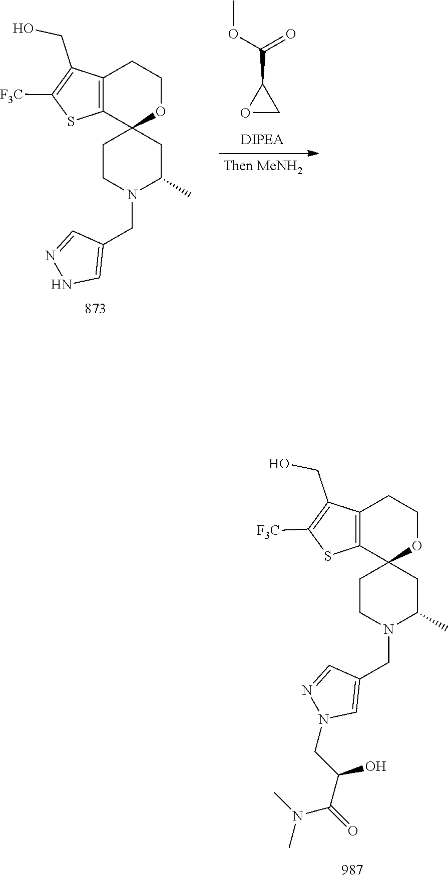

The title compound was prepared in a similar procedure as 985 with N-methyl methanamine in MeOH (2.4 mL of 2 M, 4.8 mmol) as the nucleophile in the second step. And the second step was performed at RT for 24 h. Purification by silica gel chromatography (Gradient: 0-12% MeOH in DCM) yielded a mixture of product and starting material. Subsequent purification by reversed-phase HPLC. Method: Waters XSelect CSH Prep Phenyl Hexyl Column; 30×150 mm, 5 micron. Gradient: Acetonitrile in Water with 0.2% Formic Acid. Product was isolated as (2R)-2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N,N-dimethyl-propanamide (formic acid salt) (7.7 mg, 14%). 1H NMR (300 MHz, MeOD) δ 7.85 (s, 1H), 7.64 (s, 1H), 4.85-4.81 (m, 1H), 4.59 (s, 2H), 4.52-4.27 (m, 3H), 4.11 (d, J=14.2 Hz, 1H), 4.04-3.91 (m, 2H), 3.44-3.32 (m, 1H), 3.26-3.01 (m, 5H), 2.94 (s, 3H), 2.75 (t, J=5.5 Hz, 2H), 2.29 (t, J=13.0 Hz, 2H), 2.12-1.82 (m, 2H), 1.46 (d, J=6.4 Hz, 3H). LCMS m/z 517.02 [M+1]⁺.

### Compound 988

### Ethyl 2-hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanoate (988)

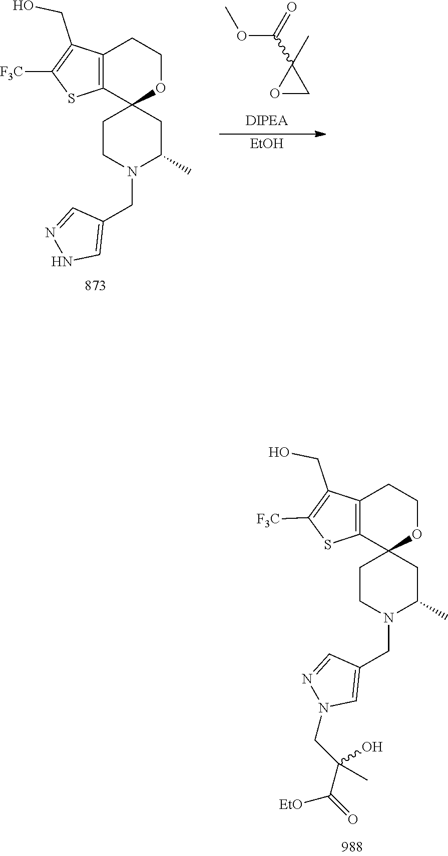

To a 2-dram vial was added [(2′S,7R)-2′-methyl-1′-(1H-pyrazol-4-ylmethyl)-2-(trifluoromethyl)spiro[4,5-dihydrothieno[2,3-c]pyran-7,4′-piperidine]-3-yl]methanol (124 mg, 0.30 mmol), methyl 2-methyloxirane-2-carboxylate (51 μL, 0.48 mmol), EtOH (1.5 mL) and DIPEA (270 μL, 1.55 mmol). The reaction vial was heated at 90° C. for 20 h. The reaction was concentrated and purified by silica gel chromatography (Gradient: 0-14% MeOH in dichloromethane) to provide ethyl 2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanoate (100 mg, 59%). 1H NMR (300 MHz, CDCl3) δ 7.41 (s, 1H), 7.35 (s, 1H), 4.64 (d, J=4.7 Hz, 2H), 4.47 (d, J=13.8 Hz, 1H), 4.29 (d, J=8.4 Hz, 1H), 4.24-4.11 (m, 3H), 4.08-3.66 (m, 3H), 3.51 (d, J=14.3 Hz, 1H), 2.80-2.37 (m, 5H), 1.99 (d, J=10.4 Hz, 2H), 1.83 (t, J=12.0 Hz, 1H), 1.65 (t, J=12.5 Hz, 1H), 1.47 (s, 3H), 1.32-1.22 (m, 3H), 1.18 (d, J=6.1 Hz, 3H). LCMS m/z 532.26 [M+1]⁺.

### Compound 989

### 2-Hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanamide (989)

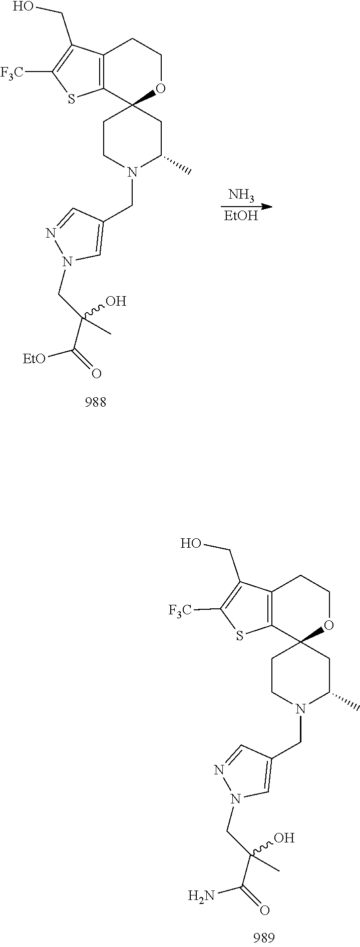

To a 2-dram vial was added ethyl 2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanoate (25 mg, 0.047 mmol), EtOH (0.1 mL) and ammonia in MeOH (750 μL of 4 M, 3.0 mmol). The reaction mixture was stirred at RT for 20 h and then heated to 60° C. for 60 h. The reaction was concentrated and purified by reversed-phase HPLC. Method: Waters XSelect CSH C18 OBD Prep Column; 30×150 mm, 5 micron. Gradient: Acetonitrile in Water with 0.2% Formic Acid. Product was isolated as 2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanamide (formic acid salt) (12 mg, 29%). 1H NMR (300 MHz, MeOD) δ 7.82 (d, J=2.2 Hz, 1H), 7.61 (s, 1H), 4.59 (s, 2H), 4.50 (d, J=14.1 Hz, 1H), 4.39 (d, J=14.0 Hz, 1H), 4.26 (d, J=14.0 Hz, 1H), 4.14 (d, J=14.1 Hz, 1H), 3.96 (q, J=5.2 Hz, 2H), 3.43-3.33 (m, 1H), 3.16 (d, J=14.2 Hz, 2H), 2.75 (t, J=5.5 Hz, 2H), 2.29 (t, J=13.4 Hz, 2H), 2.15-1.78 (m, 2H), 1.46 (d, J=6.4 Hz, 3H), 1.38 (s, 3H). LCMS m/z 503.29 [M+1]⁺.

### Compound 990

### 2-Hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N,2-dimethyl-propanamide (990)

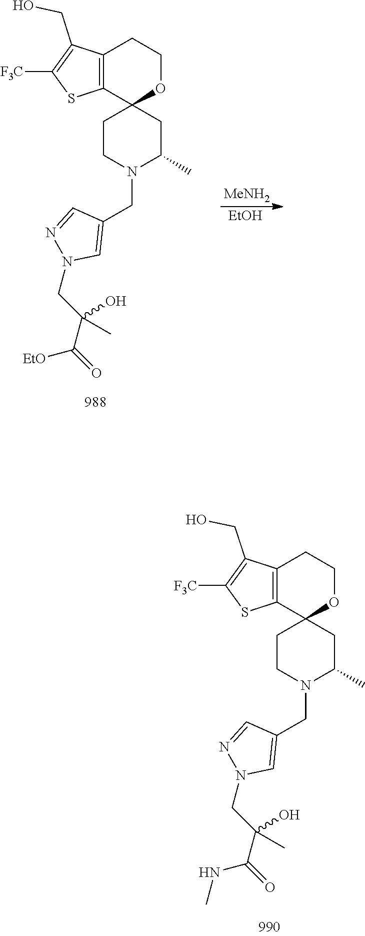 [SMILES: "MeNH2" ; "EtOH" ; CCOC(=O)C(C)(O)Cn1cc(CN2CC[C@]3(C[C@@H]2C)OCCc2c3sc(C(F)(F)F)c2CO)cn1 ; CNC(=O)C(C)(O)Cn1cc(CN2CC[C@]3(C[C@@H]2C)OCCc2c3sc(C(F)(F)F)c2CO)cn1]

The title compound was prepared in a similar procedure as 989 with methyl amine in EtOH (600 μL of 8 M, 4.8 mmol) as the nucleophile in the second step. And the second step took 36 h. Final product was isolated as 2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-N,2-dimethyl-propanamide (formic acid salt) (16.9 mg, 34%). 1H NMR (300 MHz, MeOD) δ 7.84 (d, J=2.8 Hz, 1H), 7.63 (s, 1H), 4.59 (s, 2H), 4.49 (d, J=14.0 Hz, 2H), 4.25 (dd, J=13.9, 4.7 Hz, 2H), 3.98 (t, J=5.0 Hz, 2H), 3.48 (s, 1H), 3.29-3.16 (m, 2H), 2.76 (t, J=5.5 Hz, 2H), 2.66 (s, 3H), 2.33 (t, J=16.8 Hz, 2H), 2.14-1.88 (m, 2H), 1.52 (d, J=6.4 Hz, 3H), 1.37 (s, 3H). LCMS m/z 517.33 [M+1]⁺.

### Compound 991

### N-cyclopropyl-2-hydroxy-3-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanamide (991)

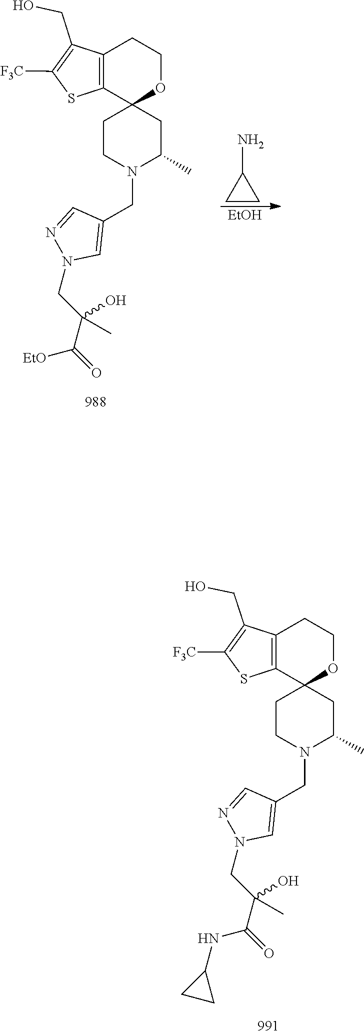

The title compound was prepared in a similar procedure as 989 with cyclopropanamine (210 μL, 3.03 mmol) as the nucleophile in the second step. Purification by silica gel chromatography (Gradient: 0-20% MeOH in dichloromethane) to provide N-cyclopropyl-2-hydroxy-3-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]pyrazol-1-yl]-2-methyl-propanamide (5.8 mg, 14%). 1H NMR (300 MHz, CDCl3) δ 7.45 (s, 1H), 7.37 (s, 1H), 6.87 (s, 1H), 5.62 (s, 1H), 4.64 (s, 2H), 4.54 (dd, J=13.7, 8.5 Hz, 1H), 4.11 (dd, J=13.5, 8.9 Hz, 1H), 3.92 (dq, J=15.4, 9.8, 7.6 Hz, 3H), 3.49 (s, 4H), 2.70 (d, J=5.9 Hz, 3H), 2.65-2.42 (m, 3H), 2.01 (d, J=12.5 Hz, 2H), 1.95-1.67 (m, 2H), 1.36 (t, J=4.2 Hz, 3H), 0.39 (d, J=11.2 Hz, 1H), 0.33-0.17 (m, 1H). LCMS m/z 543.2 [M+1]⁺.

### Compound 992

### (2R)-2-hydroxy-3-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]propanamide (992)

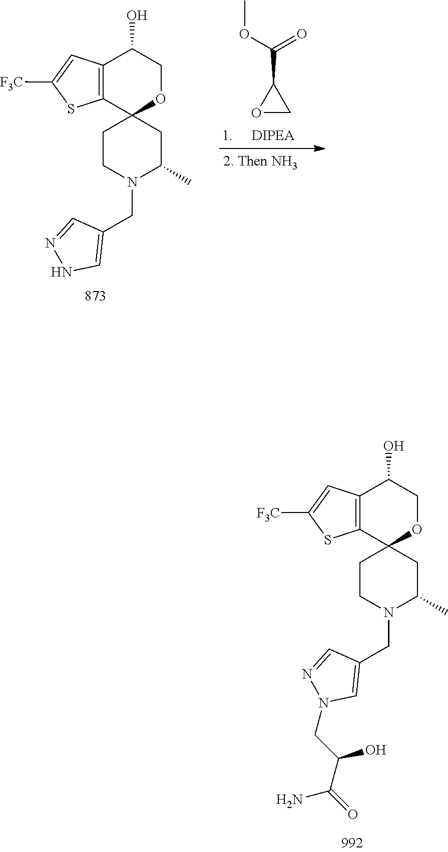

### Step 1: Synthesis of ethyl (2R)-2-hydroxy-3-[(2′S,4S,7R)-4-hydroxy-2′-methyl-2-(trifluoromethyl)spiro[4,5-dihydrothieno[2,3-c]pyran-7,4′-piperidine]-1′-yl]propanoate

To 2-dram vial was added (2′S,4S,7R)-2′-methyl-1′-(1H-pyrazol-4-ylmethyl)-2-(trifluoromethyl)spiro[4,5-dihydrothieno[2,3-c]pyran-7,4′-piperidine]-4-ol (93 mg, 0.23 mmol), methyl (2R)-oxirane-2-carboxylate (40 μL, 0.46 mmol), EtOH (1 mL) and DIPEA (210 μL, 1.21 mmol). The reaction was heated to 90° C. for 29 h, and additional 20 uL epoxide and 100 uL DIPEA were added. The reaction vial was heated for another 20 h. The reaction was concentrated and purified by silica gel chromatography (Gradient: 0-14% MeOH in dichloromethane) to provide ethyl (2R)-2-hydroxy-3-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]propanoate (62.7 mg, 54%). 1H NMR (300 MHz, CDCl3) δ 7.41 (s, 1H), 7.38 (s, 1H), 7.34 (d, J=1.4 Hz, 1H), 4.65-4.33 (m, 4H), 4.24 (q, J=7.1 Hz, 2H), 4.01-3.72 (m, 3H), 3.54 (d, J=14.3 Hz, 1H), 3.47 (s, 1H), 2.73-2.57 (m, 2H), 2.43 (td, J=12.3, 2.6 Hz, 1H), 2.13-2.01 (m, 1H), 1.95 (dt, J=13.7, 3.0 Hz, 1H), 1.86-1.65 (m, 2H), 1.29 (t, J=7.1 Hz, 3H), 1.18 (d, J=6.2 Hz, 3H). LCMS m/z 504.13 [M+1]⁺.

### Step 2: Synthesis of (2R)-2-hydroxy-3-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]propenamide (992)

Ethyl (2R)-2-hydroxy-3-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]propanoate (62.7 mg) from step 1 was dissolved in EtOH (0.4 mL). Then a EtOH (100 μL) (¼) was transferred to a 2-dram vial. Ammonia in MeOH (570 μL of 4 M, 2.280 mmol) was added. The reaction mixture was stirred at RT. After 14 h, the reaction mixture was concentrated and purification by reversed-phase HPLC. Method: Waters XSelect CSH Prep Phenyl Hexyl Column; 30×150 mm, 5 micron. Gradient: Acetonitrile in Water with 0.2% Formic Acid. Product was isolated as (2R)-2-hydroxy-3-[4-[[(2′S,4S,7R)-4-hydroxy-2′-methyl-2-(trifluoromethyl)spiro[4,5-dihydrothieno[2,3-c]pyran-7,4′-piperidine]-1′-yl]methyl]pyrazol-1-yl]propenamide (12 mg, 37%). 1H NMR (300 MHz, MeOD) δ7.85 (s, 1H), 7.63 (s, 1H), 7.48 (s, 1H), 4.63-4.44 (m, 2H), 4.44-4.28 (5, 3H), 4.13 (d, J=14.1 Hz, 1H), 3.98 (dd, (=12.3, 3.5 Hz, 1H), 3.82 (dd, J=12.3, 3.9 Hz, 1H), 3.41 (s, 1H), 3.26-3.03 (m, 2H), 2.35 (t, J=14.2 Hz, 2H), 2.06-1.83 (m, 2H), 1.48 (d, J=6.4 Hz, 3H). LCMS m/z 475.2 [M+1]⁺.

### Compounds 993-999

Compounds 993-999 (see Table 45) were prepared from intermediate 873 following procedures preparing compound 992. Epoxides and amines were obtained from commercial sources. Any modifications to methods are noted in Table 45 and accompanying footnotes.

TABLE 45
Structure and physicochemical data for compounds 993-999

| | | Epoxide | Amine | Starting | ¹H NMR; LCMS m/z |
|---|---|---|---|---|---|
| Cmpd | Structure | Reagent | Reagent | Material | [M + H]⁺ |
| 993 | 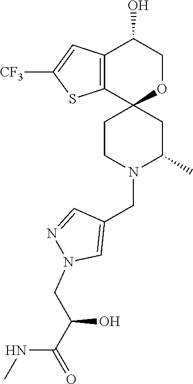 | 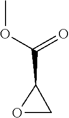 | MeNH₂ | 873 | 1H NMR (300 MHz, MeOD) δ 7.83 (s, 1H), 7.62 (s, 1H), 7.48 (s, 1H), 4.60-4.46 (m, 2H), 4.45-4.25 (m, 3H), 4.12 (d, J = 14.1 Hz, 1H), 3.98 (dd, J = 12.3, 3.5 Hz, 1H), 3.81 (dd, J = 12.3, 3.9 Hz, 1H), 3.40 (s, 1H), 3.28- 3.00 (m, 2H), 2.73 (s, 3H), 2.35 (t, J = 14.7 Hz, 2H), 1.96 (t, J = 13.2 Hz, 2H), 1.48 (d, J = 6.4 Hz, 3H); LCMS m/z 489.25 [M + H]⁺ |
| 994 | 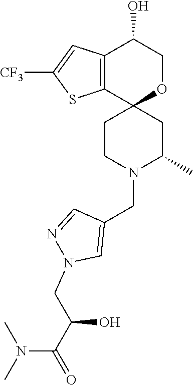 | 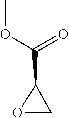 | Me₂NH | 873¹ | 1H NMR (300 MHz, MeOD) δ 7.65 (s, 1H), 7.52 (s, 1H), 7.44 (s, 1H), 4.84-4.80 (m, 1H), 14.52 (s, 1H), 4.39 (dd, J = 14.0, 5.0 Hz, 1H), 4.27 (dd, J = 14.1, 7.1 Hz, 1H), 3.95 (dd, J = 12.4, 3.6 Hz, 2H), 3.76 (dd, J = 12.1, 4.0 Hz, 2H), 3.03 (s, 3H), 2.93 (s, 3H), 2.73 (d, J = 44.6 Hz, 3H), 2.17 (d, J = 14.2 Hz, 2H), 1.76 (d, J = 12.5 Hz, 2H), 1.29 (d, J = 6.1 Hz, 3H); LCMS m/z 503.33 [M + H]⁺ |
| 995 | 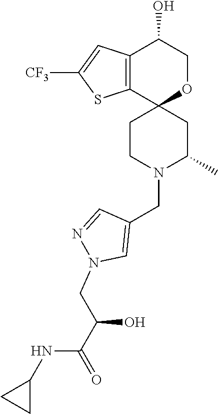 [SMILES: C[C@H]1C[C@@]2(CCN1Cc1cnn(C[C@@H](O)C(=O)NC3CC3)c1)OC[C@@H](O)c1cc(C(F)(F)F)sc12] | 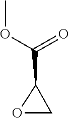 | 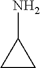 | 873² | 1H NMR (300 MHz, MeOD) δ 7.87 (s, 1H), 7.65 (s, 1H), 7.49 (s, 1H), 4.57 (t, J = 3.7 Hz, 1H), 4.54-4.41 (m, 2H), 4.35 (d, J = 10.6 Hz, 2H), 4.21 (d, J = 14.0 Hz, 1H), 3.99 (dd, J = 12.3, 3.4 Hz, 1H), 3.83 (dd, J = 12.3, 3.9 Hz, 1H), 3.54 (s, 1H), 3.38-3.31 (m, 1H), 3.22 (t, J = 12.3 Hz, 1H), 2.62 (dt, J = 7.3, 3.9 Hz, 1H), 2.40 (t, J = 15.7 Hz, 2H), 2.17-1.85 (m, 2H), 1.53 (d, J = 6.4 Hz, 3H), 0.72 (td, J = 7.0, 4.8 Hz, 2H), 0.49 (p, J = 4.4 Hz, 2H); LCMS m/z 515.3 [M + H]⁺ |
| 996 | 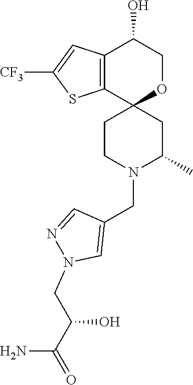 [SMILES: C[C@H]1C[C@@]2(CCN1Cc1cnn(C[C@H](O)C(N)=O)c1)OC[C@@H](O)c1cc(C(F)(F)F)sc12] | 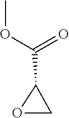 | NH₃ | 873³ | 1H NMR (300 MHz, MeOD) δ 7.90 (s, 1H), 7.67 (s, 1H), 7.49 (s, 1H), 4.63-4.55 (m, 1H), 4.55-4.47 (m, 2H), 4.38 (d, J = 11.5 Hz, 2H), 4.24 (d, J = 14.1 Hz, 1H), 3.99 (dd, J = 12.3, 3.4 Hz, 1H), 3.83 (dd, J = 12.3, 3.8 Hz, 1H), 3.56 (s, 1H), 3.35 (s, 1H), 3.29-3.15 (m, 1H), 2.40 (t, J = 14.9 Hz, 2H), 2.21-1.93 (m, 2H), 1.54 (d, J = 6.4 Hz, 3H); LCMS m/z 475.29 [M + H]⁺ |
| 997 | 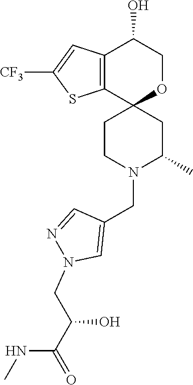 | 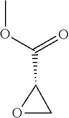 | MeNH₂ | 873³ | 1H NMR (300 MHz, MeOD) δ 7.88 (s, 1H), 7.66 (s, 1H), 7.49 (s, 1H), 4.64-4.44 (m, 3H), 4.44-4.29 (m, 2H), 4.24 (d, J = 14.1 Hz, 1H), 3.99 (dd, J = 12.3, 3.4 Hz, 1H), 3.83 (dd, J = 12.4, 3.8 Hz, 1H), 3.54 (s, 1H), 3.35 (s, 1H), 3.29-3.16 (m, 1H), 2.73 (s, 3H), 2.40 (t, J = 15.0 Hz, 2H), 2.18-1.89 (m, 2H), 1.54 (d, J = 6.4 Hz, 3H); LCMS m/z 488.12 [M + H]⁺ |
| 998 | 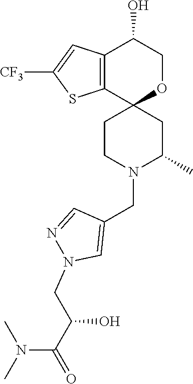 | 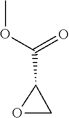 | Me₂NH | 873^{3,4} | 1H NMR (300 MHz, MeOD) δ 7.86 (s, 1H), 7.65 (s, 1H), 7.49 (s, 1H), 4.86-4.80 (m, 1H), 4.57 (t, J = 3.5 Hz, 1H), 4.45 (dd, J = 13.8, 5.5 Hz, 2H), 4.31 (dd, J = 14.1, 7.0 Hz, 1H), 4.15 (d, J = 14.0 Hz, 1H), 3.98 (dd, J = 12.4, 3.5 Hz, 1H), 3.82 (dd, J = 12.3, 3.9 Hz, 1H), 3.45 (s, 1H), 3.28- 3.12 (m, 2H), 3.11 (s, 3H), 2.94 (s, 3H), 2.37 (t, J = 15.4 Hz, 2H), 1.98 (t, J = 13.2 Hz, 2H), 1.50 (d, J = 6.4 Hz, 3H); LCMS m/z 503.33 [M + H]+ |
| 999 | 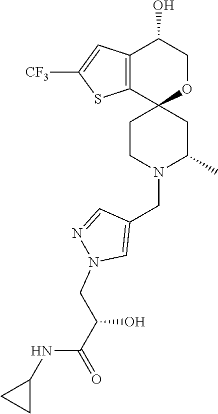 [SMILES: C[C@H]1C[C@@]2(CCN1Cc1cnn(C[C@H](O)C(=O)NC3CC3)c1)OC[C@@H](O)c1cc(C(F)(F)F)sc12] | 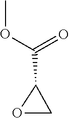 | 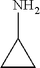 | 873^{3,4} | 1H NMR (300 MHz, MeOD) δ 7.78 (s, 1H), 7.59 (s, 1H), 7.48 (s, 1H), 4.84-4.79 (m, 1H), 4.55 (d, J = 3.7 Hz, 1H), 4.51-4.41 (m, 1H), 4.34 (d, J = 9.5 Hz, 3H), 4.10- 3.92 (m, 2H), 3.81 (dd, J = 12.3, 3.8 Hz, 1H), 3.07 (d, J = 30.8 Hz, 2H), 2.63 (dd, J = 7.5, 3.7 Hz, 1H), 2.32 (t, J = 13.7 Hz, 2H), 1.90 (t, J = 13.0 Hz, 2H), 1.44 (d, J = 6.4 Hz, 3H), 0.71 (d, J = 6.2 Hz, 2H), 0.49 (d, J = 3.9 Hz, 2H); LCMS m/z 515.38 [M + H]⁺ |

Footnotes:
1) Reaction time was 30 h. Purification by reversed-phase HPLC. Method: Waters XSelect CSH C18 OBD Prep Column; 30 × 150 mm, 5 micron. Gradient: Acetonitrile in Water with 10 mM Ammonium Hydroxide.
2) Reaction was performed at 60° C. for 26 h.
3) Purification by reversed-phase HPLC. Method: Waters XSelect CSH C18 OBD Prep Column; 30 × 150 mm, 5 micron. Gradient: Acetonitrile in Water with 0.2% Formic Acid.
4) Reaction was performed at 60° C. for 4 d.

### Preparation S76

### [(2′S, 7R)-2′-methyl-1′-prop-2-ynyl-2-(trifluoromethyl)spiro[4,5-dihydrothieno[2,3-c]pyran-7,4′-piperidine]-3-yl]methanol (S76)

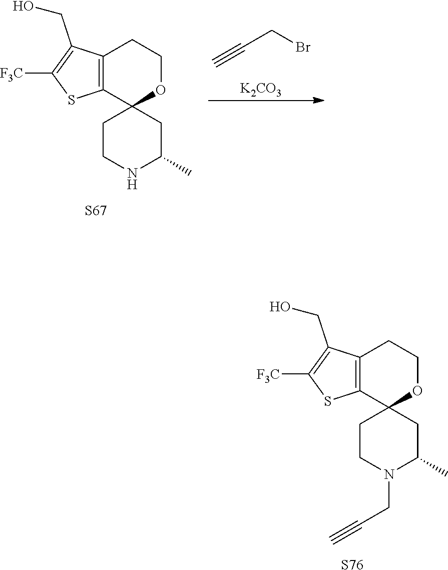

A mixture of [(2′S)-2′-methyl-2-(trifluoromethyl)spiro[4,5-dihydrothieno[2,3-c]pyran-7,4′-piperidine]-3-yl]methanol S67 (740 mg, 2.303 mmol) and potassium carbonate (330 mg, 2.388 mmol) in THF (15 mL) was heated to 50° C. Propargyl bromide (285 μL, 3.198 mmol) in toluene was added and the mixture was stirred at 50° C. After 24 h, the reaction was quenched with aqueous NaHCO₃ solution and DCM. The organic layer was separated through a phase separator. The solvent was removed in vacuo. The concentrated oil was rediluted in minimal DCM and loaded onto a SiO₂ column for purification (Gradient: 0-20% MeOH in DCM) to yield [(2′S,7R)-2′-methyl-1′-prop-2-ynyl-2-(trifluoromethyl)spiro[4,5-dihydrothieno[2,3-c]pyran-7,4′-piperidine]-3-yl]methanol (600 mg, 72%). ¹H NMR (300 MHz, Chloroform-d) δ 4.62-4.35 (m, 2H), 3.80 (hept, J=6.0 Hz, 2H), 3.49 (dd, J=17.4, 2.4 Hz, 1H), 3.24 (dd, J=17.4, 2.4 Hz, 1H), 2.87-2.43 (m, 5H), 2.17-1.65 (m, 4H), 1.47 (dd, J=13.7, 11.4 Hz, 2H), 0.92 (d, J=6.3 Hz, 3H).

### Compound 1000

### 2-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]triazol-1-yl]-2-methyl-propan-1-ol (1000)

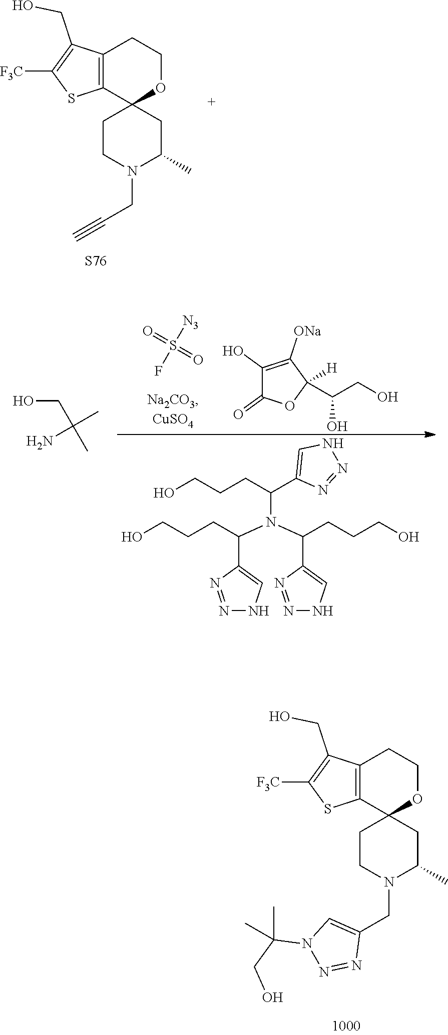

### Standard Method H: Click Chemistry to Prepare Azides

To a 1-dram vial containing 2-amino-2-methyl-propan-1-ol (22.3 mg, 0.25 mmol) was added Na₂CO₃ aqueous solution (250 μL, 1 M, 0.25 mmol). Fluorosulfonyl azide solution (550 uL, ˜0.45M in MTBE, ˜250 umol, prepared according the protocol in Nature, 574, 2019, 86-89.) was added to the vial. The resulting solution was stirred at room temperature for 20 minutes. [(2′S,7R)-2′-methyl-1′-prop-2-ynyl-2-(trifluoromethyl)spiro[4,5-dihydrothieno[2,3-c]pyran-7,4′-piperidine]-3-yl]methanol S76 (30 mg, 0.835 mmol) was dissolved in DMSO (1 mL) and added to the reaction vial. Aqueous CuSO₄ solution (85 uL, 0.1 M, 8.5 umol) was added to the vial. A solution of 4-[bis[4-hydroxy-1-(1H-triazol-4-yl)butyl]amino]-4-(1H-triazol-4-yl)butan-1-ol in DMSO (85 uL, 0.1 M, 8.5 umol) was added, followed by the addition of aqueous sodium ascorbate (2R)-2-[(1S)-1,2-dihydroxyethyl]-4-hydroxy-5-oxo-2H-furan-3-olate (85 uL, 0.2 M, 17 umol). The resulting reaction mixture was heated at 50° C. open to the air overnight. An additional CuSO₄ (100 uL, 0.1 M) and sodium ascorbate (100 uL, 0.2 M) were added, and heated was continued overnight. The vial was cooled to room temperature, diluted with water (2 mL) and DCM (1 mL), and stirred for several minutes. The mixture was passed through a filtration plate and washed with DCM (1 mL), and the organic layer was evaporated. The resulting residue was dissolved in DMSO (1 mL), and purified by reversed-phase HPLC. Method: C18 Waters Sunfire column (30×150 mm, 5 micron) with 10 mM ammonium hydroxide as the modifier. Product was isolated as 2-[4-[[(2′S,7R)-3-(hydroxymethyl)-2′-methyl-2-(trifluoromethyl)spiro[4,5-dihydrothieno[2,3-c]pyran-7,4′-piperidine]-1′-yl]methyl]triazol-1l-yl]-2-methyl-propan-1-ol (6.9 mg, 17%). ¹H NMR (400 MHz, DMSO-d₆) δ 7.98 (s, 1H), 5.21 (d, J 33.7 Hz, 2H), 4.43 (s, 2H), 3.92-3.79 (m, 3H), 3.64-3.57 (s, 3H), 2.67-2.60 (m, 3H), 2.50-2.34 (i, 2H), 1.96 (d, J=13.2 Hz, 2H), 1.71 (dt, J=13.6, 7.0 Hz, 1H), 1.54 (s, 6H), 1.49 (t, J=12.6 Hz, 1H), 1.15 (d, J=6.2 Hz, 3H). LCMS m/z 475.16 [M+1]⁺.

### Compounds 1001-1005

Compounds 1001-1005 (see Table 46) were prepared in a single step from intermediate S76 using Standard Method H. Amines were obtained from commercial sources. Any modifications to methods are noted in Table 46 and accompanying footnotes.

TABLE 46
Structure and physicochemical data for compounds 1001-1005

| | | Amine | |
|---|---|---|---|
| Cmpd | Product | Reagent | ¹H NMR; LCMS m/z [M + H]⁺ |
| 1001 | 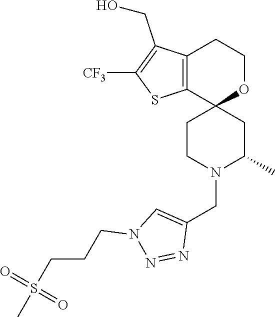 [SMILES: C[C@H]1C[C@@]2(CCN1Cc1cn(CCCS(C)(=O)=O)nn1)OCCc1c2sc(C(F)(F)F)c1CO] | 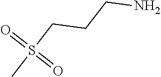 | ¹H NMR (400 MHz, DMSO-d₆) δ 8.07 (s, 1H), 5.24 (s, 1H), 4.48 (t, J = 7.0 Hz, 2H), 4.43 (d, J = 3.7 Hz, 2H), 3.91-3.69 (m, 4H), 3.15- 3.07 (m, 2H), 3.00 (d, J = 1.6 Hz, 3H), 2.63 (t, J = 5.7 Hz, 3H), 2.50- 2.39 (m, 2H), 2.26 (p, J = 6.9 Hz, 2H), 1.96 (d, J = 13.1 Hz, 2H), 1.72 (dt, J = 13.3, 6.8 Hz, 1H), 1.49 (dd, J = 24.3, 12.0 Hz, 1H), 1.14 (d, J = 6.1 Hz, 3H); LCMS m/z 523.16 [M + H]⁺ |
| 1002 | 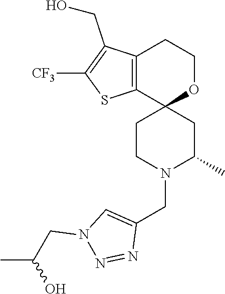 [SMILES: CC(O)Cn1cc(CN2CC[C@]3(C[C@@H]2C)OCCc2c3sc(C(F)(F)F)c2CO)nn1] | 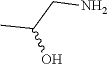 | ¹H NMR (300 MHz, Methanol-d₄) δ 7.94 (s, 1H), 4.67-3.62 (m, 9H), 2.91-2.36 (m, 5H), 2.23-1.52 (m, 4H), 1.42-0.87 (m, 6H); LCMS m/z 461.12 [M + H]⁺ |
| 1003 | 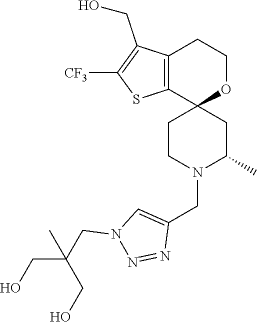 [SMILES: C[C@H]1C[C@@]2(CCN1Cc1cn(CC(C)(CO)CO)nn1)OCCc1c2sc(C(F)(F)F)c1CO] | 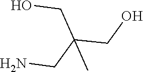 | ¹H NMR (400 MHz, DMSO-d₆) δ 7.87 (s, 1H), 5.24 (s, 1H), 4.75 (s, 2H), 4.43 (s, 2H), 4.26 (s, 2H), 3.91-3.68 (m, 4H), 3.28-3.15 (m, 4H), 2.63 (t, J = 5.6 Hz, 2H), 2.60-2.33 (m, 3H), 1.95 (t, J = 10.1 Hz, 2H), 1.70 (td, J = 13.0, 4.4 Hz, 1H), 1.47 (dd, J = 13.7, 11.2 Hz, 1H), 1.14 (d, J = 6.1 Hz, 3H), 0.66 (s, 3H); LCMS /z 505.19 [M + H]⁺ |
| 1004 | 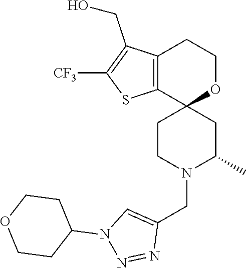 [SMILES: C[C@H]1C[C@@]2(CCN1Cc1cn(C3CCOCC3)nn1)OCCc1c2sc(C(F)(F)F)c1CO] | 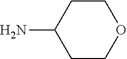 | ¹H NMR (300 MHz, Methanol-d₄) δ 8.03 (s, 1H), 4.77 (ddd, J = 15.8, 9.1, 7.0 Hz, 1H), 4.57 (d, J = 1.4 Hz, 2H), 4.16-3.76 (m, 6H), 3.76-3.48 (m, 2H), 2.88-2.52 (m, 5H), 2.27- 1.77 (m, 7H), 1.64 (dd, J = 13.9, 11.4 Hz, 1H), 1.26 (d, J = 6.2 Hz, 3H); LCMS m/z 487.17 [M + H]⁺ |
| 1005 | 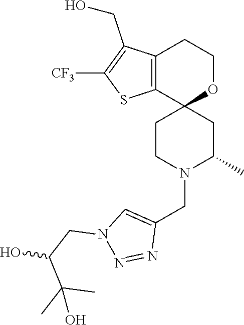 [SMILES: C[C@H]1C[C@@]2(CCN1Cc1cn(CC(O)C(C)(C)O)nn1)OCCc1c2sc(C(F)(F)F)c1CO] | 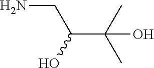 | ¹H NMR (400 MHz, DMSO-d₆) δ 7.90 (d, J = 3.3 Hz, 1H), 5.22 (s, 1H), 5.11 (d, J = 6.4 Hz, 1H), 4.70- 4.51 (m, 2H), 4.42 (s, 2H), 4.21- 4.03 (m, 1H), 3.84 (dt, J = 11.0, 6.4 Hz, 3H), 3.68 (dd, J = 14.5, 3.7 Hz, 1H), 3.53 (d, J = 7.3 Hz, 1H), 2.62 (t, J = 5.7 Hz, 3H), 2.44 (d, J = 11.9 Hz, 2H), 1.95 (d, J = 13.2 Hz, 2H), 1.79-1.62 (m, 1H), 1.47 (t, J = 12.4 Hz, 1H), 1.20-1.01 (m, 9H); LCMS m/z 505.19 [M + H]⁺ |

### Compound 1006

### Methyl (2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylate (1006)

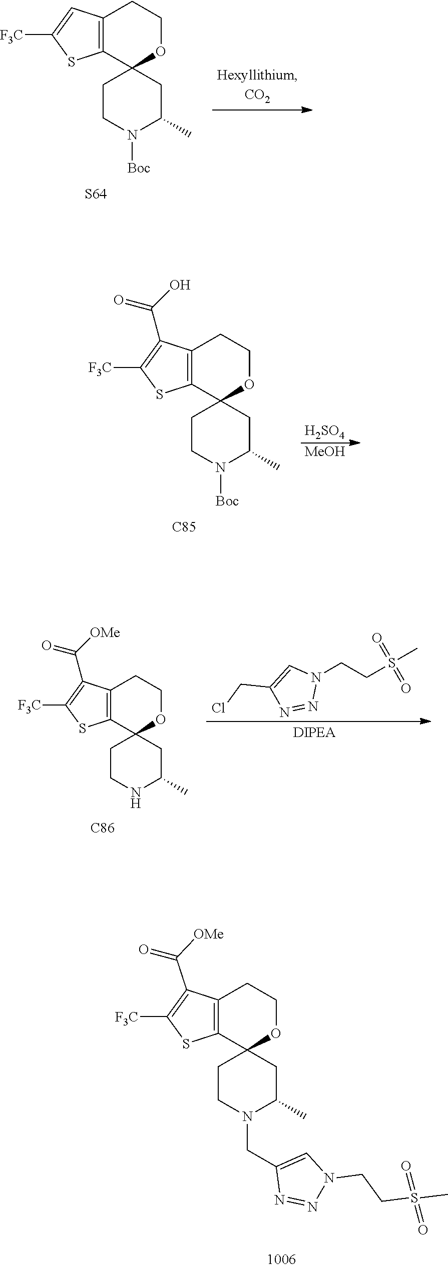

### Step 1. Synthesis of (2′S)-1′-tert-butoxycarbonyl-2′-methyl-2-(trifluoromethyl)spiro[4,5-dihydrothieno[2,3-c]pyran-7,4′-piperidine]-3-carboxylic acid (C85)

Tert-butyl (2′S)-2′-methyl-2-(trifluoromethyl)spiro[4,5-dihydrothieno[2,3-c]pyran-7,4′-piperidine]-1′-carboxylate (530 mg, 1.151 mmol) S64 was dissolved in THF (10 mL) and cooled to −78° C. and hexyllithium (800 μL of 2.3 M, 1.840 mmol) was added. The reaction mixture was stirred at −78° C. for 3 min and CO₂ was bubbled through. After 10 min. the reaction was warmed to rt. The reaction mixture was diluted with EtOAc and 1N NaOH, and the aqueous layer was acidified with 6N HCl and extracted with EtOAc. The combined organic layers were concentrated in vacuo and purified by silica gel chromatography (Gradient: 0 to 50% EtOAc in heptane with 1% AcOH) to give the product as a yellow oil. Further purification by C18 reverse phase HPLC (CH₃CN/H₂O, TFA modifier) provided pure fractions, which were diluted with 0.5N HCl/EtOAc and the organic layer was dried and concentrated to give the product as a white solid. (2′S)-1′-tert-butoxycarbonyl-2′-methyl-2-(trifluoromethyl)spiro[4,5-dihydrothieno[2,3-c]pyran-7,4′-piperidine]-3-carboxylic acid (226 mg, 45%). ¹⁹F NMR (282 MHz, DMSO-d₆) δ−52.52 (d, J=1.3 Hz), LCMS m/z 436.43 [M+1]⁺.

### Step 2. Synthesis of methyl (2′S)-2′-methyl-2-(trifluoromethyl)spiro[4,5-dihydrothieno[2,3-c]pyran-7,4′-piperidine]-3-carboxylate (C86)

(2′S)-1′-tert-butoxycarbonyl-2′-methyl-2-(trifluoromethyl)spiro[4,5-dihydrothieno[2,3-c]pyran-7,4′-piperidine]-3-carboxylic acid C85 (224 mg, 0.5144 mmol) was dissolved in MeOH (8 mL) and H₂SO₄ (200 μL, 3.752 mmol) was added. The reaction mixture was heated to 60° C. for 24 h and stirred at room temperature for 48 h. The reaction mixture was diluted with EtOAc/1N NaOH and the organic layer dried and concentrated in vacuo to provide methyl (2′S)-2′-methyl-2-(trifluoromethyl)spiro[4,5-dihydrothieno[2,3-c]pyran-7,4′-piperidine]-3-carboxylate (162 mg, 90%). ¹H NMR (300 MHz, Chloroform-d) δ 3.95 (td, J=5.7, 3.6 Hz, 2H), 3.91 (s, 3H), 3.18-3.04 (m, 2H), 2.95 (dd, J=12.4, 4.0 Hz, 1H), 2.88 (t, J=5.6 Hz, 2H), 2.06 (d, J=13.5 Hz, 2H), 1.72 (td, J=13.2, 4.7 Hz, 1H), 1.40 (dd, J=13.4, 11.3 Hz, 1H), 1.09 (d, J=6.4 Hz, 3H). LCMS m/z 349.65 [M+1]⁺.

### Step 3. Synthesis of methyl (2′S, 7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylate (1006)

Methyl (2′S)-2′-methyl-2-(trifluoromethyl)spiro[4,5-dihydrothieno[2,3-c]pyran-7,4′-piperidine]-3-carboxylate C86 (160 mg, 0.4580 mmol) was dissolved in 1,2-dichloroethane (2 mL), with DIPEA (160 μL, 0.9186 mmol) and 4-(chloromethyl)-1-(2-methylsulfonylethyl)triazole (120 mg, 0.5365 mmol). The tube was sealed, and the reaction mixture was heated to 60° C. for 72 h. The reaction mixture was purified by reverse phase HPLC (Column: C18, TFA modifier). The pure fractions were combined and diluted with 1N NaOH and EtOAc and the organic layer was dried and concentrated to give the product as a white solid. Methyl (2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylate (155 mg, 59%). ¹H NMR (300 MHz, Chloroform-d) δ 7.65 (s, 1H), 4.93-4.81 (m, 2H), 4.13-3.99 (m, 1H), 3.98-3.80 (m, 6H), 3.75 (t, J=6.3 Hz, 2H), 2.85 (t, J=5.5 Hz, 2H), 2.82-2.55 (m, 6H), 2.12-1.97 (m, 2H), 1.97-1.79 (m, 1H), 1.75-1.67 (m, 1H), 1.26 (d, J=6.2 Hz, 3H). LCMS m/z 537.54 [M+1]⁺.

### Preparation S77

### (2′S, 7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylic acid (S77)

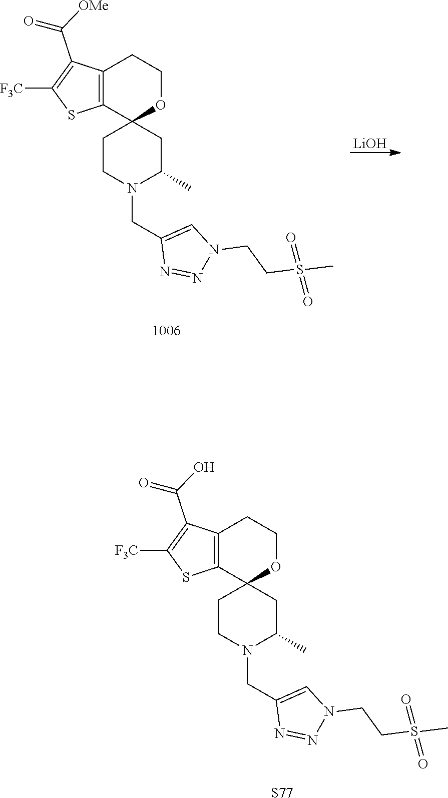 [SMILES: COC(=O)c1c(C(F)(F)F)sc2c1CCO[C@@]21CCN(Cc2cn(CCS(C)(=O)=O)nn2)[C@@H](C)C1 ; "LiOH" ; C[C@H]1C[C@@]2(CCN1Cc1cn(CCS(C)(=O)=O)nn1)OCCc1c2sc(C(F)(F)F)c1C(=O)O ; "S77"]

Methyl (2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylate 1006 (122 mg, 0.2137 mmol) was dissolved in THF (2 mL) and MeOH (2 mL), and LiOH (220 μL of 1 M, 0.2200 mmol) added. The reaction mixture was stirred for 24 h. More LiOH (80 μL of 1 M, 0.08000 mmol) was added and the reaction mixture stirred overnight. Additional LiOH (100 μL of 1 M, 0.1000 mmol) was added and the reaction mixture stirred for 6 h. The reaction mixture was then concentrated in vacuo to provide the crude product. (2′S,7R)-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine]-3-carboxylic acid (Lithium Ion (1)) (112 mg, 98%). LCMS m/z 523.18 [M+1]⁺.

### Compound 1007

### 2-[4-[[(2′S, 7R)-3-(hydroxymethyl)-2′-methyl-2-(trifluoromethyl)spiro[4,5-dihydrothieno[2,3-c]pyran-7,4′-piperidine]-1′-yl]methyl]triazol-1-yl]-2-methyl-propan-1-ol (1007)

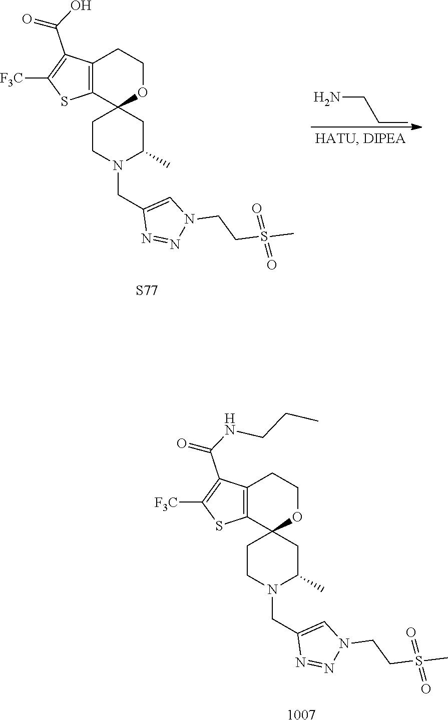

Standard Method I: Amide Coupling with HATU

(2′S,7R)-2′-methyl-1′-[[1-(2-methylsulfonylethyl)triazol-4-yl]methyl]-2-(trifluoromethyl) spiro[4,5-dihydrothieno[2,3-c]pyran-7,4′-piperidine]-3-carboxylic acid (Lithium Ion (1)) S77 (18 mg, 0.034 mmol) and propan-1-amine (13.9 μL, 0.169 mmol) were dissolved in DMF (1 mL) and HATU (15 mg, 0.0.039 mmol) was added, followed by DIPEA (5.8 μL, 0.034 mmol). The reaction mixture was stirred at rt for overnight. The reaction mixture was filtered. Purification by reversed-phase HPLC. Method: C18 Waters Sunfire column (30×150 mm, 5 micron). Gradient: MeCN in H₂O with 0.1% trifluoroacetic acid. The final product was isolated as (2′S,7R)-2′-methyl-1′-[[1-(2-methylsulfonylethyl)triazol-4-yl]methyl]-N-propyl-2-(trifluoromethyl)spiro[4,5-dihydrothieno[2,3-c]pyran-7,4′-piperidine]-3-carboxamide (trifluoroacetic acid salt) (8.8 mg, 47%). LCMS m/z 564.31 [M+1]⁺.

### Compounds 1008-1009

Compounds 1008-1009 (see Table 47) were prepared in a single step from intermediate S77 using Standard Method I (As in the preparation of compound 1007). Amines were obtained from commercial sources. Any modifications to methods are noted in Table 47 and accompanying footnotes.

TABLE 47
Structure and physicochemical data for compounds 1008-1009

| | | Amine | ¹H NMR; LCMS m/z |
|---|---|---|---|
| Cmpd | Structure | Reagent | [M + H]⁺ |
| 1008 | 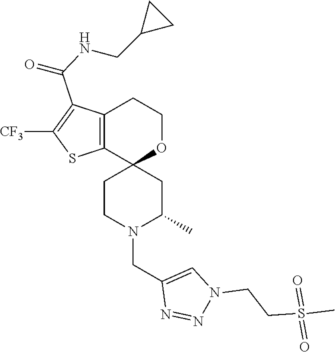 [SMILES: C[C@H]1C[C@@]2(CCN1Cc1cn(CCS(C)(=O)=O)nn1)OCCc1c2sc(C(F)(F)F)c1C(=O)NCC1CC1] | 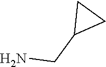 | LCMS m/z 576.37 [M + H]⁺ |
| 1009 | 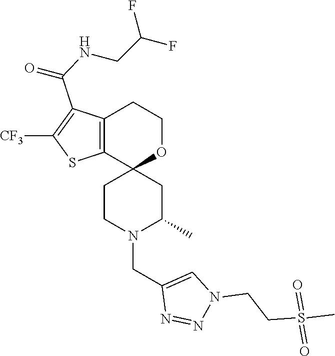 [SMILES: C[C@H]1C[C@@]2(CCN1Cc1cn(CCS(C)(=O)=O)nn1)OCCc1c2sc(C(F)(F)F)c1C(=O)NCC(F)F] | 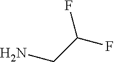 | LCMS m/z 586.3 [M + H]⁺ |

### Compound 1010

### (2′S, 7R)-3-chloro-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine](Trifluoroacetate salt) (1010)

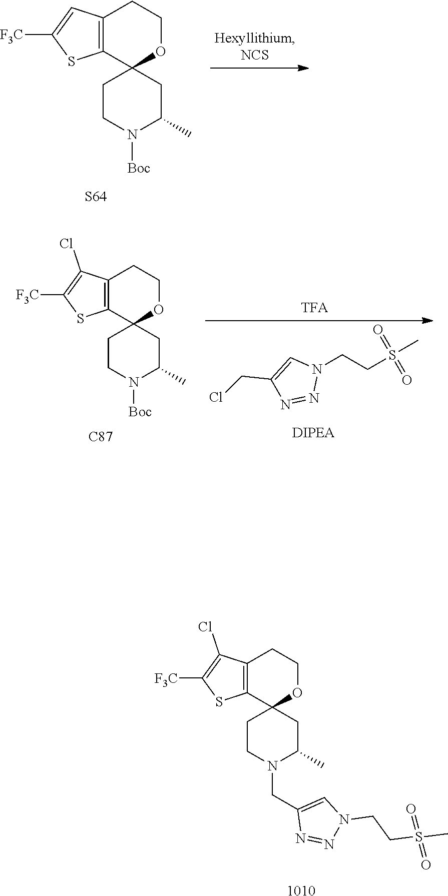

### Step 1. Synthesis of tert-butyl (2′S, 7R)-3-chloro-2′-methyl-2-(trifluoromethyl)spiro[4,5-dihydrothieno[2,3-c]pyran-7,4′-piperidine]-1′-carboxylate (C87)

Tert-butyl (2′S,7R)-2′-methyl-2-(trifluoromethyl)spiro[4,5-dihydrothieno[2,3-c]pyran-7,4′-piperidine]-1′-carboxylate S64 (150 mg, 0.3832 mmol) was dissolved in THF (5 mL) and cooled to −78° C. and hexyllithium (250 μL of 2 M, 0.5000 mmol) was added. The reaction mixture was stirred for 5 min and NCS (80 mg, 0.5991 mmol) dissolved in 1 mL THF was added. The reaction mixture was warmed to room temperature. The reaction mixture was diluted with 1N NaOH/EtOAc and the organic layer was dried and concentrated. The crude material was purified by reverse phase HPLC (Column: C18). The pure fractions were diluted with 1N NaOH and EtOAc, and the organic layer was dried and concentrated to give the product as an oil: tert-butyl (2′S,7R)-3-chloro-2′-methyl-2-(trifluoromethyl)spiro[4,5-dihydrothieno[2,3-c]pyran-7,4′-piperidine]-1′-carboxylate (17 mg, 10%). ¹H NMR (300 MHz, Chloroform-d) δ 3.96-3.84 (m, 1H), 3.84-3.76 (m, 2H), 3.69-3.55 (m, 1H), 3.28-3.14 (m, 1H), 2.46 (t, J=5.5 Hz, 2H), 2.13-1.91 (m, 2H), 1.74-1.50 (m, 2H), 1.36 (s, 9H), 1.14 (d, J=6.7 Hz, 3H).

### Step 2. Synthesis of (2′S, 7R)-3-chloro-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine](Trifluoroacetate salt) (1010)

Tert-butyl (2′S,7R)-3-chloro-2′-methyl-2-(trifluoromethyl)spiro[4,5-dihydrothieno[2,3-c]pyran-7,4′-piperidine]-1′-carboxylate C87 (17 mg, 0.03992 mmol) was dissolved in DCM (1 mL) and TFA (0.5 mL, 6.490 mmol) was added. After 20 min, the reaction mixture was concentrated to and redissolved in DCE (1 mL). To this solution, 4-(chloromethyl)-1-(2-methylsulfonylethyl)triazole (13 mg, 0.05812 mmol) was added followed by DIPEA (30 μL, 0.1722 mmol). The reaction mixture was heated overnight at 60° C. Additional 4-(chloromethyl)-1-(2-methylsulfonylethyl)triazole (8 mg, 0.03577 mmol) was added and the reaction mixture was heated to 60° C. overnight. The reaction mixture was purified with silica gel chromatography (Gradient: 0 to 10% MeOH in DCM). Further purification by reversed-phase HPLC. Method: C18 Waters Sunfire column (30×150 mm, 5 micron). Gradient: MeCN in H₂O with 0.1% trifluoroacetic acid. The product was isolated as a white solid: (2′S,7R)-3-chloro-2′-methyl-1′-[[1-(2-methylsulfonylethyl)triazol-4-yl]methyl]-2-(trifluoromethyl)spiro[4,5-dihydrothieno[2,3-c]pyran-7,4′-piperidine](Trifluoroacetate salt) (9 mg, 35%). ¹H NMR (300 MHz, Chloroform-d) δ 8.25 (s, 1H), 4.97 (q, J=6.5 Hz, 2H), 4.74 (d, J=14.0 Hz, 1H), 4.26 (d, J=13.9 Hz, 1H), 4.06-3.79 (m, 2H), 3.79-3.63 (m, 2H), 3.63-3.45 (m, 1H), 3.42-3.14 (m, 2H), 2.95 (s, 3H), 2.68-2.59 (m, 2H), 2.49-2.26 (m, 2H), 2.23-2.07 (m, 2H), 1.64 (d, J=6.4 Hz, 3H). LCMS m/z 513.14 [M+1]⁺.

### Compound 1011

### (2′S, 7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine](1011)

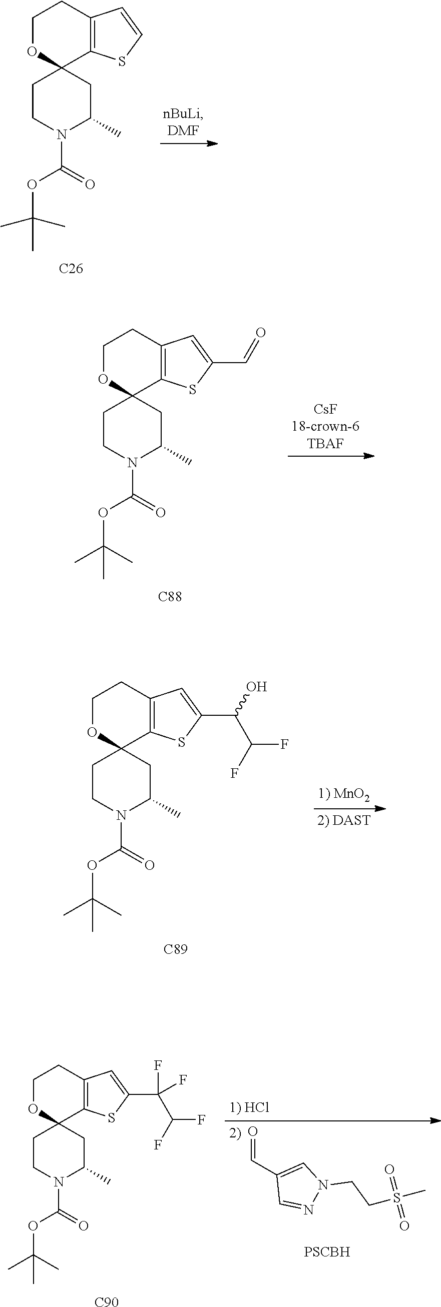

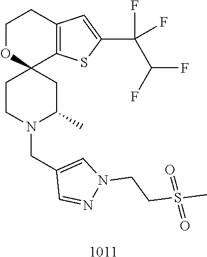 [SMILES: C[C@H]1C[C@@]2(CCN1Cc1cnn(CCS(C)(=O)=O)c1)OCCc1cc(C(F)(F)C(F)F)sc12 ; "1011"]

### Step 1. Synthesis of tert-butyl (2′S, 7R)-2-formyl-2′-methyl-spiro[4,5-dihydrothieno[2,3-c]pyran-7,4′-piperidine]-1′-carboxylate (C88)

To a solution of tert-butyl (2′S,7R)-2′-methylspiro[4,5-dihydrothieno[2,3-c]pyran-7,4′-piperidine]-1′-carboxylate C26 (81.4 g, 252 mmol) in THF (740 mL) at −78° C. under nitrogen atmosphere was added butyllithium (120 mL of 2.6 M, 310 mmol) over the course of 20 min. After stirring for 70 min, DMF (100 mL, 1.3 mol) was added over the course of 5 min. The mixture was stirred at −78° C. for 30 min and stirred for an additional 45 min at 0° C. The reaction was quenched by the addition of saturated aqueous ammonium chloride solution (600 mL). To the mixture was added EtOAc (1 L) and water (500 mL), and the organic layer was separated and washed with saturated aqueous ammonium chloride solution (600 mL), water (600 mL), and brine (600 mL). The organic layer was dried over magnesium sulfate, filtered, and concentrated in vacuo to give the product as amber oil (88 g, 99%). ¹H NMR (300 MHz, Chloroform-d) δ 9.83 (s, 1H), 7.42 (s, 1H), 4.01 (ddd, J=11.5, 6.7, 5.3 Hz, 1H), 3.94-3.82 (m, 2H), 3.75 (ddd, J=14.0, 6.1, 4.7 Hz, 1H), 3.35 (ddd, J=14.1, 8.8, 5.4 Hz, 1H), 2.71 (td, J=5.5, 2.8 Hz, 2H), 2.28-2.06 (m, 2H), 1.90-1.65 (m, 2H), 1.47 (s, 9H), 1.26 (d, J=6.6 Hz, 3H). LCMS m/z 252.08 [M−Boc]⁺.

### Step 2. Synthesis of tert-butyl (2′S, 7R)-2-(2,2-difluoro-1-hydroxy-ethyl)-2′-methyl-spiro[4,5-dihydrothieno[2,3-c]pyran-7,4′-piperidine]-1′-carboxylate (C89)

Under nitrogen atmosphere, cesium fluoride (295 mg, 1.94 mmol) and 1,4,7,10,13,16-hexaoxacyclooctadecane (515 mg, 1.95 mmol) were added to a solution of tert-butyl (2′S,7R)-2-formyl-2′-methyl-spiro[4,5-dihydrothieno[2,3-c]pyran-7,4′-piperidine]-1′-carboxylate C88 (680 mg, 1.9 mmol) in 1,2-dimethoxyethane (27 mL) followed by difluoromethyl(trimethyl)silane (972 mg, 7.83 mmol). The resulting mixture was stirred at room temperature for 3 h. Next, tetrabutylammonium fluoride (2 mL of 1 M, 2 mmol) was added, and the mixture was stirred for additional 30 min. The mixture was partitioned between water and dichloromethane, the organic layer was collected and concentrated in vacuo. Purification by silica gel chromatography (Gradient: 0-60% EtOAc in heptane) yielded the product (471 mg, 60%). ¹H NMR (300 MHz, Chloroform-d) δ 6.79 (d, J=2.7 Hz, 1H), 5.81 (td, J=55.9, 4.5 Hz, 1H), 5.01 (td, J=10.4, 4.4 Hz, 1H), 4.06-3.68 (m, 3H), 3.39-3.08 (m, 1H), 2.73-2.54 (m, 2H), 2.26-2.03 (m, 2H), 1.91-1.67 (m, 2H), 1.49 (d, J=1.3 Hz, 9H), 1.32-1.23 (m, 4H). LCMS m/z 403.7 [M+H]⁺.

### Step 3. Synthesis of tert-butyl (2′S, 7R)-2′-methyl-2-(1,1,2,2-tetrafluoroethyl)spiro[4,5-dihydrothieno[2,3-c]pyran-7,4′-piperidine]-1′-carboxylate (C90)

To a solution of tert-butyl (2′S,7R)-2-(2,2-difluoro-1-hydroxy-ethyl)-2′-methyl-spiro[4,5-dihydrothieno[2,3-c]pyran-7,4′-piperidine]-1′-carboxylate C89 (471 mg, 1.17 mmol) in DCM (5 mL) was added MnO₂ (985 mg, 11.3 mmol). The mixture was stirred overnight and filtered through a pad of Celite®, and the filtrate was concentrated in vacuo. The residue was dissolved in DCM (2 mL), and diethylaminosulfur trifluoride (770 μL, 5.8 mmol) was added. The mixture was stirred at 40° C. for 3 hours. Water was added, and the organic layer was separated and concentrated in vacuo. Purification by silica gel chromatography (Gradient: 0-30% EtOAc in heptane) afforded the product (221 mg, 45%). ¹H NMR (300 MHz, Methanol-d₄) δ 7.13 (t, J=1.4 Hz, 1H), 6.30 (tt, J=53.4, 3.1 Hz, 1H), 4.07 (dt, J=11.2, 6.2 Hz, 1H), 3.90 (t, J=5.5 Hz, 2H), 3.84-3.62 (m, 1H), 2.68 (t, J=5.5 Hz, 2H), 2.38-2.09 (m, 2H), 1.95-1.64 (m, 2H), 1.48 (s, 9H), 1.22 (d, J=6.5 Hz, 3H). Note: one of the proton resonances is obscured by signals corresponding to residual NMR solvent. LCMS m/z 424.32 [M+H]⁺.

### Step 4. Synthesis of (2′S, 7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine](1011)

To a solution of tert-butyl (2′S)-2′-methyl-2-(1,1,2,2-tetrafluoroethyl)spiro[4,5-dihydrothieno[2,3-c]pyran-7,4′-piperidine]-1′-carboxylate C90 (36 mg, 0.085 mmol) in 1,4-dioxane (200 μL) was added HCl (430 μL of 4 M in 1,4-dioxane, 1.7 mmol). The mixture was stirred at room temperature for 30 min and concentrated in vacuo. The residue was dissolved in DCM (2 mL), and 1-(2-methylsulfonylethyl)pyrazole-4-carbaldehyde (25 mg, 0.12 mmol) was added, followed by acetic acid (24 mg, 0.40 mmol) and polymer-supported cyanoborohydride (127 mg, 0.254 mmol). The mixture was heated at 95° C. in a microwave reactor for 45 min and filtered. Concentration of the filtrate in vacuo and purification by silica gel chromatography (Gradient: 0-20% MeOH in DCM) yielded the product. (11 mg, 23%). ¹H NMR (300 MHz, Methanol-d₄) δ 7.77 (s, 1H), 7.57 (s, 1H), 7.14 (d, J=1.4 Hz, 1H), 6.30 (tt, J=53.4, 3.0 Hz, 1H), 4.64 (t, J=6.4 Hz, 2H), 4.06-3.57 (m, 6H), 2.92-2.58 (m, 7H), 2.21-2.02 (m, 2H), 2.01-1.82 (m, 2H), 1.72 (dd, J=14.0, 11.6 Hz, 1H), 1.28 (d, J=6.3 Hz, 3H). LCMS m/z 510.12 [M+H]⁺.

### Compound 1012

### (2′S, 7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine]-4-ol (1012)

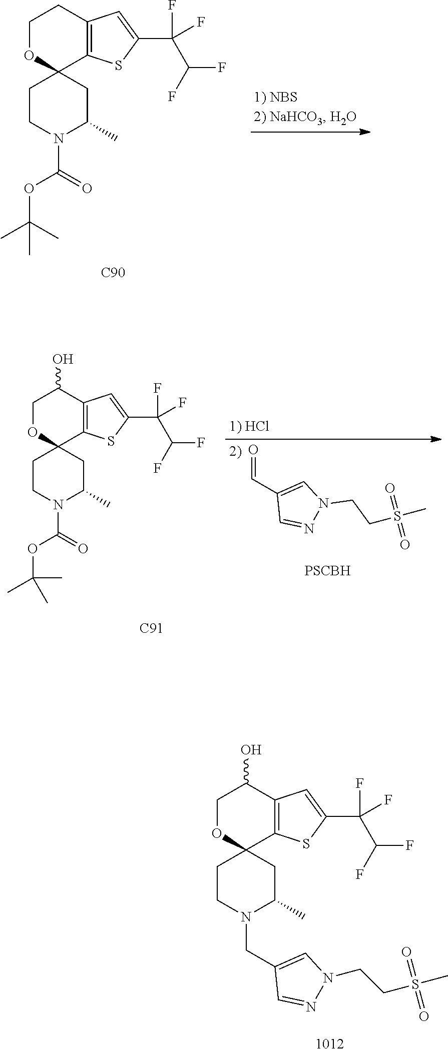

### Step 1. Synthesis of tert-butyl (2′S)-4-hydroxy-2′-methyl-2-(1,1,2,2-tetrafluoroethyl)spiro[4,5-dihydrothieno[2,3-c]pyran-7,4′-piperidine]-1′-carboxylate (C91)

To a solution of tert-butyl (2′S)-2′-methyl-2-(1,1,2,2-tetrafluoroethyl)spiro[4,5-dihydrothieno[2,3-c]pyran-7,4′-piperidine]-1′-carboxylate C90 (230 mg, 0.54 mmol) in chlorobenzene (6 mL) under argon atmosphere was N-bromosuccinimide (193 mg, 1.08 mmol) and 2,6-lutidine (140 μL, 1.2 mmol). The solution was irradiated with blue LED for 30 min. The reaction was quenched with sodium bisulfate, and the aqueous layer was extracted with DCM. The organic layer was washed with brine and concentrated in vacuo. The residue was dissolved in THF (6 mL) and sodium bicarbonate (680 mg, 8.1 mmol) in water (6 mL) was added. The mixture was heated at 60° C. for 3 days. The mixture was then diluted with water and extracted with DCM. Purification by silica gel chromatography (Gradient: 0-23% ethyl acetate in heptane) yielded the product (53 mg, 22%) as a white solid. LCMS m/z 440.3 [M+H]⁺.

### Step 2. Synthesis of tert-butyl (2′S)-4-hydroxy-2′-methyl-2-(1,1,2,2-tetrafluoroethyl)spiro[4,5-dihydrothieno[2,3-c]pyran-7,4′-piperidine]-1′-carboxylate (1012)

To a solution of tert-butyl (2′S)-4-hydroxy-2′-methyl-2-(1,1,2,2-tetrafluoroethyl)spiro[4,5-dihydrothieno[2,3-c]pyran-7,4′-piperidine]-1′-carboxylate C91 (53 mg, 0.12 mmol) in 1,4-dioxane (0.7 mL) was added HCl (2 mL of 4 M, 8 mmol) in 1,4-dioxane. The reaction was stirred for 30 minutes and concentrated in vacuo. The residue was dissolved in DCM (2 mL) and 1-(2-methylsulfonylethyl)pyrazole-4-carbaldehyde (33 mg, 0.16 mmol) was added followed by polymer-supported cyanoborohydride (170 mg of 2 mmol/g, 0.34 mmol). The mixture was heated to 95° C. in a microwave reactor for 45 min. The solution was filtered, and the filtrate was concentrated in vacuo. Purification by silica gel chromatography (Gradient: 0-20% MeOH in DCM) yielded the product (2′S,7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine]-4-ol (14 mg, 21%). ¹H NMR (300 MHz, methanol-d₄) δ 7.73 (s, 1H), 7.54 (s, 1H), 7.33 (d, J=1.5 Hz, 1H), 6.32 (tt, J=53.7, 3.0 Hz, 1H), 4.66-4.47 (m, 3H), 4.00-3.55 (m, 6H), 3.37-3.31 (m, 2H), 2.85-2.46 (m, 5H), 2.20-1.52 (m, 4H), 1.24 (dd, J=6.1, 4.5 Hz, 3H). LCMS m/z 526.1 [M+H]⁺.

### Compound 1013

### (2′S,7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine]-4-ol (1013)

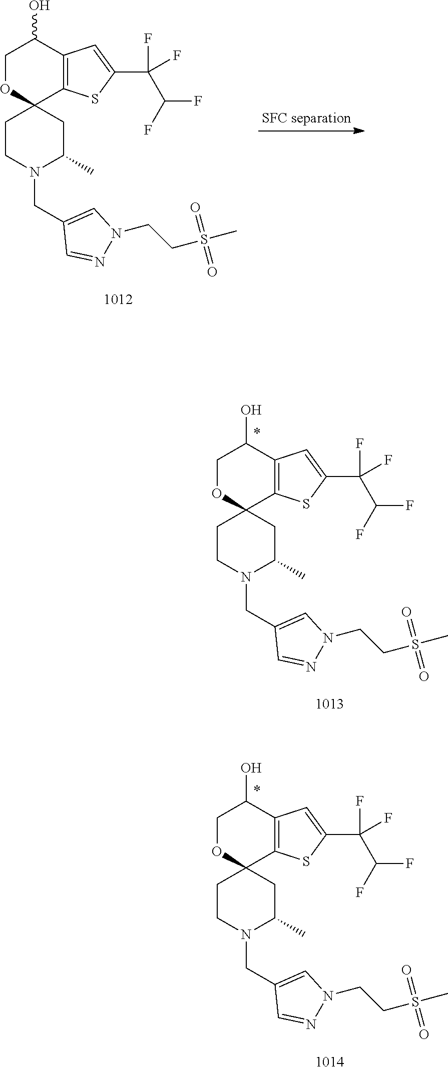

The isomeric mixture of tert-butyl (2′S)-4-hydroxy-2′-methyl-2-(1,1,2,2-tetrafluoroethyl)spiro[4,5-dihydrothieno[2,3-c]pyran-7,4′-piperidine]-1′-carboxylate 1012 (10 mg, 0.02 mmol) was separated by chiral SFC separation (2.7 mg, 27%). Column: Daicel Chiralpak® IB, 10×250 mm; Mobile Phase: 20% methanol (containing 5 mM Ammonia), 80% carbon dioxide. Flow: 15 mL/min. ¹H NMR (300 MHz, Methanol-d₄) δ 7.73 (d, J=0.8 Hz, 1H), 7.54 (d, J=0.7 Hz, 1H), 7.33 (t, J=1.4 Hz, 1H), 6.32 (tt, J=53.4, 3.0 Hz, 1H), 4.63 (dd, J=6.8, 5.9 Hz, 2H), 4.53 (t, J=4.1 Hz, 1H), 4.01-3.58 (m, 6H), 2.83-2.46 (m, 6H), 2.22-1.99 (m, 2H), 1.98-1.80 (m, 2H), 1.59 (dd, J=14.0, 11.5 Hz, 1H), 1.23 (d, J=6.3 Hz, 3H).

### Compound 1014

### (2′S, 7R)-2′-methyl-1′-[[1-(2-methylsulfonylethyl)pyrazol-4-yl]methyl]-2-(1,1,2,2-tetrafluoroethyl)spiro[4,5-dihydrothieno[2,3-c]pyran-7,4′-piperidine]-4-ol (1014)

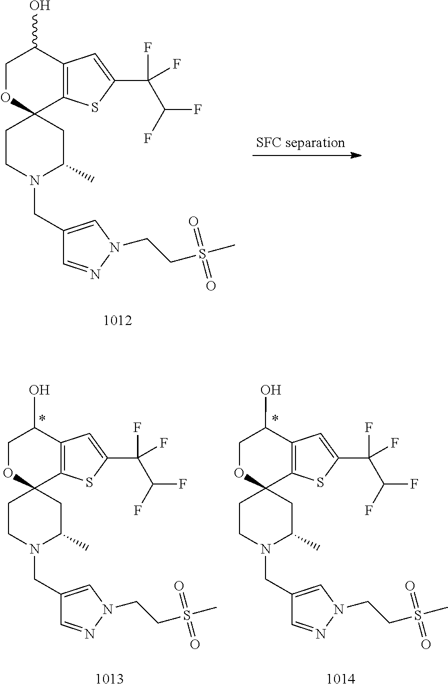

The isomeric mixture of tert-butyl (2′S)-4-hydroxy-2′-methyl-2-(1,1,2,2-tetrafluoroethyl)spiro[4,5-dihydrothieno[2,3-c]pyran-7,4′-piperidine]-1′-carboxylate JMA2 (10 mg, 0.02 mmol) was separated by chiral SFC separation (2 mg, 20%). Column: Daicel Chiralpak® IB, 10×250 mm; Mobile Phase: 20% methanol (containing 5 mM Ammonia), 80% carbon dioxide. Flow: 15 mL/min. ¹H NMR (300 MHz, Methanol-d₄) δ 7.72 (s, 1H), 7.53 (d, J=0.7 Hz, 1H), 7.33 (d, J=1.4 Hz, 1H), 6.32 (tt, J=53.4, 3.0 Hz, 1H), 4.63 (t, J=6.4 Hz, 2H), 4.51 (t, J=3.7 Hz, 1H), 4.00-3.59 (m, 6H), 2.80-2.48 (m, 5H), 2.19-1.60 (m, 4H), 1.25 (d, J=6.2 Hz, 3H).

### Compound 1015

### (2′S, 7R)-2-[deuterio(ethoxy)methyl]-2′-methyl-1′-[[1-(2-methylsulfonylethyl)pyrazol-4-yl]methyl]spiro[4,5-dihydrothieno[2,3-c]pyran-7,4′-piperidine](1015)

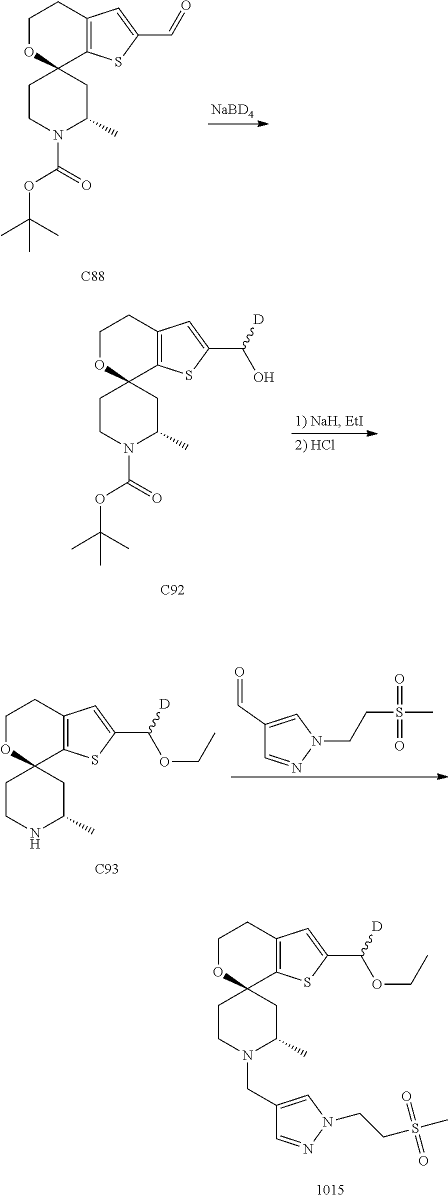

### Step 1. Synthesis of tert-butyl (2′S, 7R)-2-[deuterio(hydroxy)methyl]-2′-methyl-spiro[4,5-dihydrothieno[2,3-c]pyran-7,4′-piperidine]-1′-carboxylate (C92)

To a solution of tert-butyl (2′S,7R)-2-formyl-2′-methyl-spiro[4,5-dihydrothieno[2,3-c]pyran-7,4′-piperidine]-1′-carboxylate C88 (600 mg, 1.7 mmol) in ethanol (16 mL) was added sodium borodeuteride (65 mg, 1.7 mmol). The mixture was stirred overnight at room temperature. The mixture was diluted with water and DCM, and the aqueous layer was extracted with DCM. The organic layer was dried over sodium sulfate, filtered, and concentrated in vacuo to afford the product (605 mg, 100%) LCMS m/z 354.45 [M+H]⁺.

### Step 2. Synthesis of (2′S, 7R)-2-[deuterio(ethoxy)methyl]-2′-methyl-spiro[4,5-dihydrothieno[2,3-c]pyran-7,4′-piperidine] (C93)

To a solution of tert-butyl (2′S,7R)-2-[deuterio(hydroxy)methyl]-2′-methyl-spiro[4,5-dihydrothieno[2,3-c]pyran-7,4′-piperidine]-1′-carboxylate C92 (60 mg, 0.17 mmol) in THF (1.6 mL) was added iodoethane (41 μL, 0.51 mmol) and NaH (12 mg, 0.50 mmol). were added. The mixture was stirred at room temperature for 5 days. Then, saturated aqueous ammonium chloride solution was added, and the mixture was extracted with DCM. The organic layer was dried over sodium sulfate, filtered, and concentrated in vacuo. The residue was dissolved in 1,4-dioxane (667 μL) and HCl (655 μL of 4 M, 2.62 mmol) in 1,4-dioxane were added. After stirring for 30 min, the mixture was concentrated in vacuo. The residue was then purified by reversed phase chromatography (C18 column; Gradient: MeCN in H₂O with 0.1% trifluoroacetic acid) to afford the product (8.3 mg, 25%). ¹H NMR (300 MHz, Methanol-d₄) δ 6.74 (d, J=0.8 Hz, 1H), 4.57 (s, 1H), 3.95 (td, J=5.5, 2.0 Hz, 2H), 3.68-3.45 (m, 3H), 3.37 (dd, J=12.7, 9.8 Hz, 1H), 2.70-2.62 (m, 3H), 2.25 (ddt, J=14.9, 12.1, 2.8 Hz, 2H), 1.99 (ddd, J=15.1, 12.7, 5.6 Hz, 1H), 1.81 (dd, J=14.9, 12.3 Hz, 1H), 1.32 (dd, J=6.6, 2.0 Hz, 3H), 1.18 (td, J=7.1, 1.9 Hz, 3H). LCMS m/z 283.26 [M+H]⁺.

### Step 3. Synthesis of (2′S, 7R)-2-[deuterio(ethoxy)methyl]-2′-methyl-1′-[[1-(2-methylsulfonylethyl)pyrazol-4-yl]methyl]spiro[4,5-dihydrothieno[2,3-c]pyran-7,4′-piperidine] (1015)

To a solution of (2′S,7R)-2-[deuterio(ethoxy)methyl]-2′-methyl-spiro[4,5-dihydrothieno[2,3-c]pyran-7,4′-piperidine] C93 (85 mg, 0.27 mmol) and 1-(2-methylsulfonylethyl)pyrazole-4-carbaldehyde (78 mg, 0.39 mmol) in dichloromethane (3.5 mL) was added polymer-supported cyanoborohydride (400 mg of 2 mmol/g, 0.80 mmol). The mixture was heated to 95° C. in a microwave reactor for 45 minutes. The mixture was then filtered and stirred in MeOH for 30 min. Purification by silica gel chromatography (Gradient: 0-20% MeOH in DCM) yielded the product (45 mg, 20%). ¹H NMR (300 MHz, methanol-d₄) 7.74 (s, 1H), 7.55 (d, J=0.8 Hz, 1H), 6.68 (d, J=0.8 Hz, 1H), 4.75-4.42 (m, 3H), 3.93-3.76 (m, 3H), 3.76-3.57 (m, 4H), 3.51 (q, J=7.0 Hz, 2H), 3.33 (d, J=8.4 Hz, 6H), 2.79-2.52 (m, 9H), 1.97-1.58 (m, 2H). LCMS m/z 469.2 [M+1]⁺.

### Compound 1016

### [(2′S,7R)-2′-methyl-1′-[[1-(2-methylsulfonylethyl)pyrazol-4-yl]methyl]spiro[4,5-dihydrothieno[2,3-c]pyran-7,4′-piperidine]-2-yl]methanol (1016)

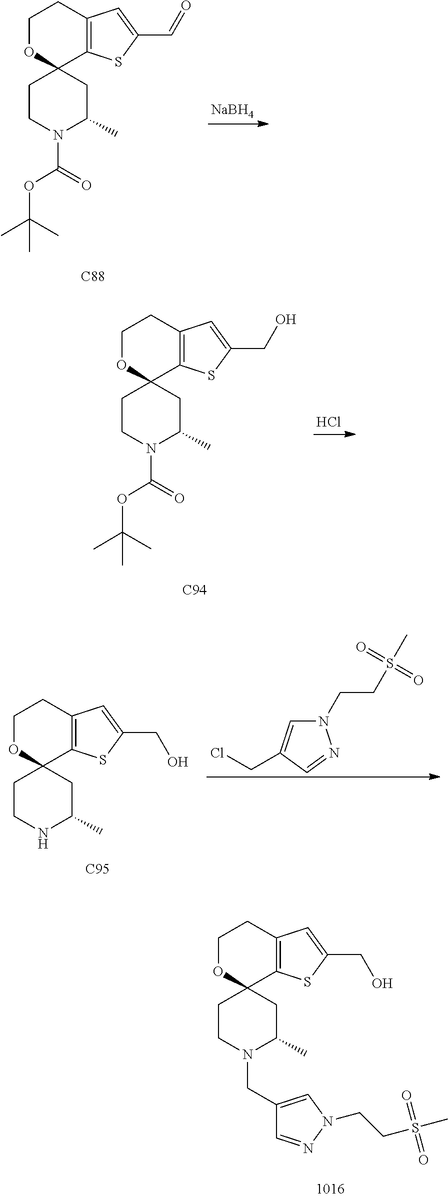

### Step 1. Synthesis of tert-butyl (2′S, 7R)-2-(hydroxymethyl)-2′-methyl-spiro[4,5-dihydrothieno[2,3-c]pyran-7,4′-piperidine]-1′-carboxylate (C94)

To a solution of tert-butyl (2′S,7R)-2-formyl-2′-methyl-spiro[4,5-dihydrothieno[2,3-c]pyran-7,4′-piperidine]-1′-carboxylate C88 (460 mg, 1.3 mmol) in MeOH (10 mL) at 0° C. was added NaBH₄ (60 mg, 1.6 mmol). After stirring for 1 h, the mixture was partitioned between saturated aqueous ammonium chloride solution and ethyl acetate. The organic layer was separated and concentrated in vacuo to give the product (434 mg, 91%) ¹H NMR (300 MHz, Chloroform-d) δ 6.67 (s, 1H), 4.81-4.73 (m, 2H), 4.07-3.95 (m, 1H), 3.88 (t, J=5.5 Hz, 2H), 3.81-3.69 (m, 1H), 3.41-3.28 (m, 1H), 2.63 (q, J=5.4 Hz, 2H), 2.27-2.04 (m, 2H), 1.91-1.68 (m, 3H), 1.51 (s, 9H), 1.28 (d, J=6.6 Hz, 3H). LCMS m/z 354.04 [M+H]⁺.

### Step 2. Synthesis of [(2′S, 7R)-2′-methylspiro[4,5-dihydrothieno[2,3-c]pyran-7,4′-piperidine]-2-yl]methanol (C95)

To a solution of tert-butyl (2′S,7R)-2-(hydroxymethyl)-2′-methyl-spiro[4,5-dihydrothieno[2,3-c]pyran-7,4′-piperidine]-1′-carboxylate C94 (28 mg, 0.077 mmol) in 1,4-dioxane was added HCl (1 mL of 4 M in 1,4-dioxane, 4 mmol). The mixture was allowed to stir at room temperature for 40 min and concentrated in vacuo. Trituration with diethyl ether and purification by reversed phase chromatography (C18 column; Gradient: MeCN in H₂O with 10 mM ammonium hydroxide) afforded the product (5 mg, 24%)¹H NMR (400 MHz, Chloroform-d) δ 6.69 (s, 1H), 4.77 (s, 2H), 4.00-3.86 (m, 2H), 3.19-3.05 (m, 2H), 3.00-2.86 (m, 1H), 2.74-2.58 (m, 2H), 2.08-1.96 (m, 3H), 1.85-1.73 (m, 1H), 1.54-1.42 (m, 1H), 1.09 (d, J=6.4 Hz, 3H). LCMS m/z 254.1 [M+H]⁺.

### Step 3. Synthesis of [(2′S, 7R)-2′-methyl-1′-[[1-(2-methylsulfonylethyl)pyrazol-4-yl]methyl]spiro[4,5-dihydrothieno[2,3-c]pyran-7,4′-piperidine]-2-yl]methanol (1016)

To a solution of [(2′S,7R)-2′-methylspiro[4,5-dihydrothieno[2,3-c]pyran-7,4′-piperidine]-2-yl]methanol C95 (35 mg, 0.14 mmol) in acetonitrile (1 mL) was added potassium carbonate (74 mg, 0.54 mmol) and 4-(chloromethyl)-1-(2-methylsulfonylethyl)pyrazole hydrogen chloride (36 mg, 0.14 mmol). The reaction mixture was heated at 70° C. for 12 h. The mixture was then partitioned between water and ethyl acetate, and the organic layer was separated and concentrated in vacuo. The residue was purified by silica gel chromatography (Gradient: 0-20% MeOH in DCM) to give the product as a white solid (13 mg, 19%). ¹H NMR (400 MHz, Chloroform-d) δ 7.54 (s, 2H), 6.68 (s, 1H), 4.75 (s, 2H), 4.66-4.57 (m, 2H), 3.94-3.79 (m, 3H), 3.72-3.60 (m, 3H), 2.79-2.52 (m, 6H), 2.50 (s, 3H), 2.05-1.93 (m, 3H), 1.90-1.75 (m, 1H), 1.26 (d, J=6.4 Hz, 3H). LCMS m/z 440.17 [M+H]⁺.

### Compound 1017

### (2′S, 7R)-2-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](1017)

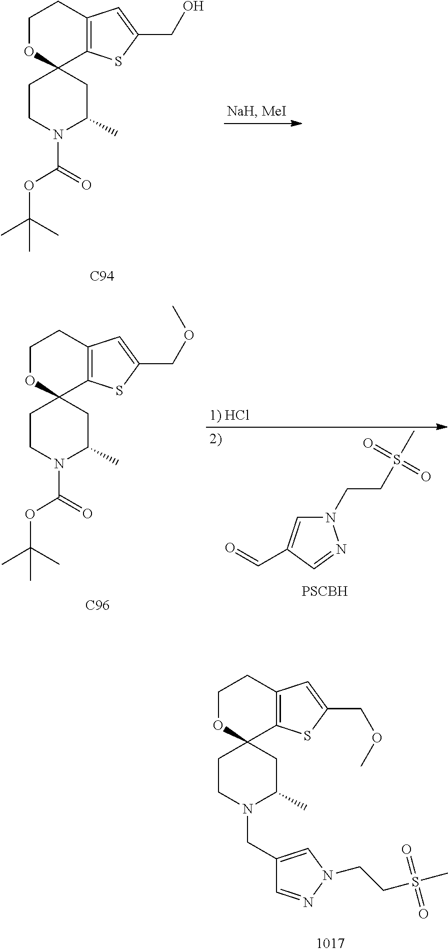

### Step 1. Synthesis of tert-butyl (2′S, 7R)-2-(methoxymethyl)-2′-methyl-spiro[4,5-dihydrothieno[2,3-c]pyran-7,4′-piperidine]-1′-carboxylate (C96)

To a solution of tert-butyl (2′S,7R)-2-(hydroxymethyl)-2′-methyl-spiro[4,5-dihydrothieno[2,3-c]pyran-7,4′-piperidine]-1′-carboxylate C94 (220 mg, 0.62 mmol) in THF (5 mL) was added NaH (26 mg of 60% w/w, 0.65 mmol). The mixture was stirred until effervescence stopped, and MeI (40 μL, 0.6 mmol) was added. The mixture was stirred at room temperature for 2 h. The mixture was quenched with water (2 mL) and extracted with diethyl ether (10 mL). The organic layer was washed with water (2×2 mL) and brine (2 mL), dried over sodium sulfate, filtered, and concentrated in vacuo. The mixture was purified by silica gel chromatography (0-30% EtOAc in heptane) to give the product (143 mg, 63%) as a colorless oil. ¹H NMR (400 MHz, Chloroform-d) δ 6.66 (s, 1H), 4.54 (d, J=0.8 Hz, 2H), 4.01 (dq, J=11.9, 6.5, 5.9 Hz, 1H), 3.91-3.88 (m, 1H), 3.75 (dt, J=13.9, 5.5 Hz, 1H), 3.41 (s, 3H), 3.36 (ddd, J=13.9, 8.7, 5.3 Hz, 1H), 2.72-2.54 (m, 2H), 2.24-2.06 (m, 2H), 1.87 (dt, J=14.5, 5.2 Hz, 1H), 1.77 (dd, J=14.3, 11.0 Hz, 1H), 1.51 (s, 9H), 1.28 (d, J=6.6 Hz, 4H). LCMS m/z 368.0 [M+H]⁺.

### Step 2. Synthesis of (2′S, 7R)-2-(methoxymethyl)-2′-methyl-1′-[[1-(2-methylsulfonylethyl)pyrazol-4-yl]methyl]spiro[4,5-dihydrothieno[2,3-c]pyran-7,4′-piperidine](1017)

To a mixture of tert-butyl (2′S,7R)-2-(methoxymethyl)-2′-methyl-spiro[4,5-dihydrothieno[2,3-c]pyran-7,4′-piperidine]-1′-carboxylate C96 (143 mg, 0.373 mmol) in diethyl ether (4 mL) was added HCl (500 μL of 4 M, 2.000 mmol) in 1,4-dioxane, and the mixture was heated to reflux for 4 h. The mixture was cooled to 0° C., and the precipitate was collected by filtration. The solid was rinsed with diethyl ether and dried in vacuo. The solid was dissolved in DCM (2 mL) and MeOH (0.2 mL), and 1-(2-methylsulfonylethyl)pyrazole-4-carbaldehyde (80 mg, 0.3 mmol) and polymer supported sodium cyanoborohydride (170 mg of 2 mmol/g, 0.34 mmol) was added. The mixture was stirred at 95° C. overnight. The mixture was then cooled to room temperature and filtered. The residue was rinsed with MeOH (2 mL), and the combined filtrate was concentrated in vacuo. Purification by silica gel chromatography (Gradient: 0-10% MeOH in DCM) afforded the product (30.7 mg, 45%) as a white solid. ¹H NMR (400 MHz, Chloroform-d) δ 7.49 (d, J=0.8 Hz, 1H), 7.44 (s, 1H), 6.63 (d, J=0.7 Hz, 1H), 4.63-4.53 (m, 2H), 4.49 (d, J=0.8 Hz, 2H), 3.88-3.76 (m, 3H), 3.66-3.56 (m, 3H), 3.36 (s, 3H), 2.67-2.46 (m, 5H), 2.43 (d, J=1.6 Hz, 3H), 2.01-1.81 (m, 3H), 1.68 (dd, J=13.8, 11.4 Hz, 1H), 1.18 (d, J=6.2 Hz, 3H). LCMS m/z 454.08 [M+H]⁺.

### Compound 1018

### [(2′S, 7R)-2′-methyl-1′-[(1-methylpyrazol-4-yl)methyl]spiro[4,5-dihydrothieno[2,3-c]pyran-7,4′-piperidine]-2-yl]methanol (1018)

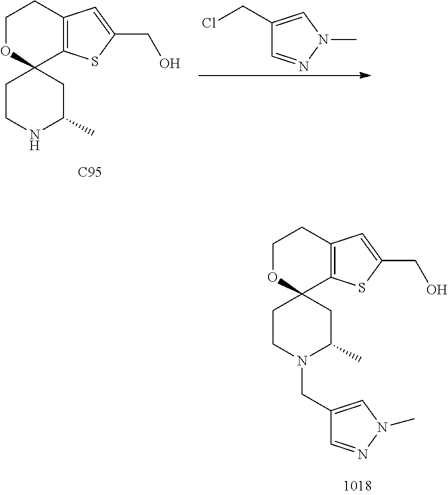

To a solution of [(2′S,7R)-2′-methylspiro[4,5-dihydrothieno[2,3-c]pyran-7,4′-piperidine]-2-yl]methanol C95 (35 mg, 0.14 mmol) in acetonitrile (1 mL) was added potassium carbonate (70 mg, 0.5 mmol) and 4-(chloromethyl)-1-methyl-pyrazole (19 mg, 0.15 mmol). The mixture was heated to 70° C. for 12 h. The mixture was partitioned between water and ethyl acetate, and the organic layer was separated and concentrated in vacuo. The residue was purified by silica gel chromatography (Gradient: 0-20% MeOH in DCM) to give the product (11 mg, 21%) as an oil. ¹H NMR (400 MHz, Chloroform-d) δ 7.43 (s, 1H), 7.37 (s, 1H), 6.66 (s, 1H), 4.75 (s, 2H), 3.96-3.78 (m, 6H), 3.59 (d, J=14.2 Hz, 1H), 2.75 (d, J=11.4 Hz, 1H), 2.72-2.51 (m, 4H), 2.07-1.92 (m, 3H), 1.83 (t, J=12.9 Hz, 1H), 1.25 (d, J=6.4 Hz, 3H). LCMS m/z 348.01 [M+H]⁺.

### Compound 1019

### [(2′S, 7R)-2′-methyl-1′-[[1-(2-methylsulfonylethyl)triazol-4-yl]methyl]spiro[4,5-dihydrothieno[2,3-c]pyran-7,4′-piperidine]-2-yl]methanol (1019)

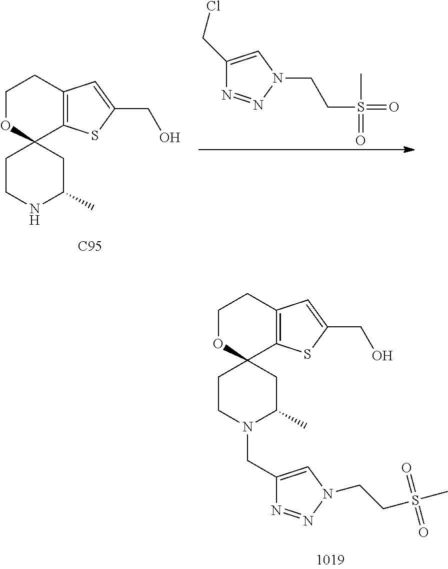 [SMILES: C[C@H]1C[C@@]2(CCN1)OCCc1cc(CO)sc12 ; CS(=O)(=O)CCn1cc(CCl)nn1 ; "C95" ; C[C@H]1C[C@@]2(CCN1Cc1cn(CCS(C)(=O)=O)nn1)OCCc1cc(CO)sc12]

To a solution of [(2′S,7R)-2′-methylspiro[4,5-dihydrothieno[2,3-c]pyran-7,4′-piperidine]-2-yl]methanol C95 (35 mg, 0.14 mmol) in acetonitrile (1 mL) was added potassium carbonate (73 mg, 0.53 mmol) and 4-(chloromethyl)-1-(2-methylsulfonylethyl)triazole hydrogen chloride (34 mg, 0.13 mmol). The mixture was heated to 70° C. for 4 h and then partitioned between water and ethyl acetate. The organic layer was separated and concentrated in vacuo. The residue was purified by silica gel chromatography (Gradient: 0-20% MeOH in DCM) to give the product (18 mg, 28%) as a white solid. ¹H NMR (400 MHz, Chloroform-d) δ 7.59 (s, 1H), 6.69 (s, 1H), 4.95 (t, J=6.6 Hz, 2H), 4.77 (s, 2H), 4.02 (d, J=14.5 Hz, 1H), 3.95-3.78 (m, 3H), 3.76-3.70 (m, 2H), 2.75 (s, 3H), 2.73-2.49 (m, 5H), 2.11-1.88 (m, 3H), 1.81-1.64 (m, 2H), 1.24 (d, J=6.2 Hz, 3H). LCMS m/z 441.07 [M+H]⁺.

### Preparation S78

### 4-(chloromethyl)-1-(2-methylsulfonylethyl)pyrazole (S78)

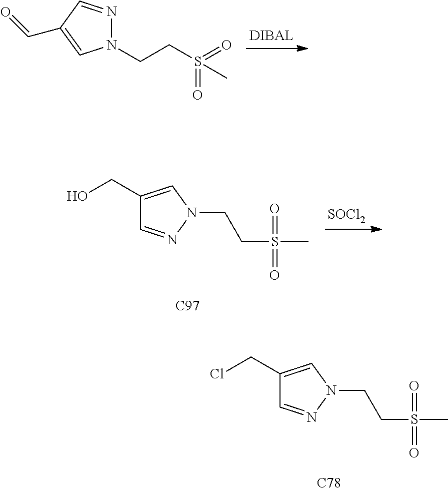

### Step 1. Synthesis of [1-(2-methylsulfonylethyl)pyrazol-4-yl]methanol (C97)

To a solution of 1-(2-methylsulfonylethyl)pyrazole-4-carbaldehyde (2 g, 9 mmol) in THF (20 mL) was added DIBAL-H in hexane (27.6 mL of 1 M, 0.0276 mol) at 0° C. The mixture was allowed to stir at room temperature 16 h. Then, the mixture was cooled to −78° C. and MeOH (25 mL) was added slowly. The mixture was concentrated in vacuo, and the residue was suspended in EtOAc (250 mL). Filtration through a pad of Celite® followed by concentration of the filtrate in vacuo afforded the product as colorless solid (1.6 g, 80%). ¹H NMR (300 MHz, DMSO-d₆) δ 7.68 (s, 1H), 7.40 (s, 1H), 4.84 (t, J=5.1 Hz, 1H), 4.48 (t, J=7.2 Hz, 2H), 4.33 (d, J=5.7 Hz, 2H), 3.65 (t, J=6.9 Hz, 2H), 2.82 (s, 3H). LCMS m/z 205.01 [M+H]⁺.

### Step 2. Synthesis of 4-(chloromethyl)-1-(2-methylsulfonylethyl)pyrazole (S78)

To a solution of [1-(2-methylsulfonylethyl)pyrazol-4-yl]methanol C97 (40 mg, 0.2 mmol) in DCM (2 mL) at 0° C. was added SOCl₂ (0.5 mL, 0.007 mol). The mixture was allowed to warm to room temperature and stirred for 2 h. The mixture was concentrated in vacuo, and the residue was triturated with diethyl ether to afford the product (42 mg, 99%) as a white solid. ¹H NMR (300 MHz, DMSO-d₆) δ 7.91 (s, 1H), 7.56 (s, 1H), 4.69 (s, 2H), 4.51 (t, J=6.9 Hz, 2H), 3.67 (t, J=7.2 Hz, 2H), 2.85 (s, 3H) as an off white solid.

### Preparation S79

### 4-(chloromethyl)-1-(2-methylsulfonylethyl)triazole (S79)

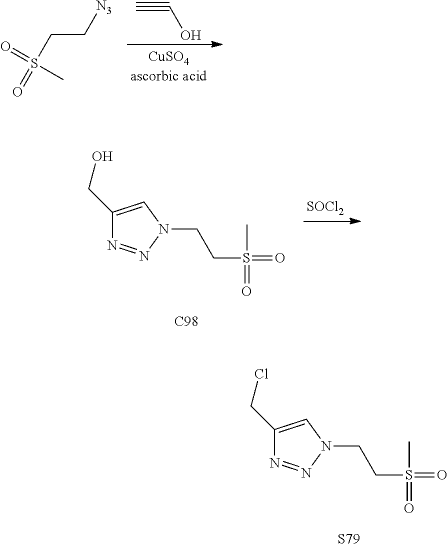

### Step 1. Synthesis of [1-(2-methylsulfonylethyl)triazol-4-yl]methanol (C98)

A mixture of prop-2-yn-1-ol (500 μL, 8.5 mmol), 1-azido-2-methylsulfonyl-ethane (1200 mg, 8.04 mmol), CuSO₄ (16 mg, 0.10 mmol), 1-(1-benzyltriazol-4-yl)-N,N-bis[(1-benzyltriazol-4-yl)methyl]methanamine (100 mg, 0.19 mmol), and ascorbic acid (1500 mg, 8.52 mmol) in MeOH (40 mL) and water (10 mL) was heated to 50° C. for 3 h. The mixture was then cooled to room temperature and concentrated in vacuo. The residue was purified by silica gel chromatography (Gradient: 0-10% MeOH in DCM) to give the product (1210 mg, 70%) as yellow solid. ¹H NMR (400 MHz, DMSO-d₆) δ 8.04 (s, 1H), 5.21 (d, J=6.8 Hz, 1H), 4.81 (t, J=7.0 Hz, 2H), 4.54 (d, J=4.5 Hz, 2H), 3.80 (t, J=6.9 Hz, 2H), 2.96 (s, 3H). LCMS m/z 206.4 [M+H]⁺.

### Step 2. Synthesis of 4-(chloromethyl)-1-(2-methylsulfonylethyl)triazole (S79)

To a solution of [1-(2-methylsulfonylethyl)triazol-4-yl]methanol C98 (2000 mg, 9.78 mmol) in dichloromethane (74 mL) and DMF (1 mL) was added thionyl chloride (1 mL, 14 mmol). The mixture was stirred for 30 min and concentrated in vacuo. The residue was triturated with diethyl ether (50 mL) and the suspension was filtered and rinsed with additional diethyl ether to afford the product (1600 mg, 73%) as a yellow solid. ¹H NMR (400 MHz, Chloroform-d) δ 7.80 (s, 1H), 4.93-4.82 (m, 2H), 4.73 (d, J=0.6 Hz, 2H), 3.74 (ddd, J=7.1, 5.5, 0.7 Hz, 2H), 2.77 (t, J=0.6 Hz, 3H). LCMS m/z 224.4 [M+H]⁺.

### Preparation 1020

### (2S,4R)-2′-(tert-butyl)-2-methyl-1-((1-methyl-1H-pyrazol-4-yl)methyl)-4′,5′-dihydrospiro[piperidine-4,7′-thieno[2,3-c]pyran] (1020)

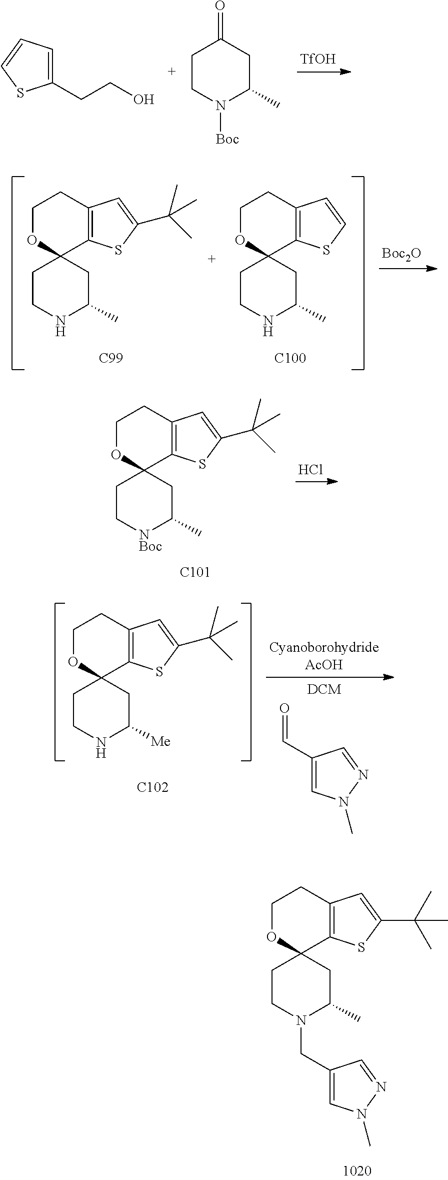

### Step 1. Synthesis of tert-butyl (2S,4R)-2′-(tert-butyl)-2-methyl-4′,5′-dihydrospiro[piperidine-4,7′-thieno[2,3-c]pyran]-1-carboxylate (C101)

To a solution of tert-butyl (S)-2-methyl-4-oxopiperidine-1-carboxylate (750 mg, 3.517 mmol) and 2-(3-thienyl)ethanol (676 mg, 5.273 mmol) in dioxane (11 mL) at 0° C. was added triflic acid (1.6 g, 10.66 mmol). The resulting solution was allowed to stir at room temperature for 3 h, after which time it was diluted with DCM and washed with 2M Na₂CO₃. The aqueous layer was extracted with DCM, and the combined organic layers were dried over sodium sulfate, then filtered and concentrated. The crude mixture containing C99 and C100 was dissolved in DCM (16 mL), and to this solution was added tert-butoxycarbonyl tert-butyl carbonate (460 mg, 2.108 mmol). The resulting solution was allowed to stir at room temperature for three hours after which time the crude product was purified by column chromatography to yield tert-butyl (2S,4R)-2′-(tert-butyl)-2-methyl-4′,5′-dihydrospiro[piperidine-4,7′-thieno[2,3-c]pyran]-1-carboxylate (82 mg, 23%). 1H NMR (300 MHz, Chloroform-d) δ 7.15 (d, J=5.1 Hz, 1H), 6.76 (d, J=5.1 Hz, 1H), 4.03 (ddd, J=11.4, 6.6, 5.1 Hz, 1H), 3.90 (t, J=5.5 Hz, 2H), 3.76 (ddd, J=13.9, 6.0, 4.9 Hz, 1H), 3.37 (ddd, J=14.0, 8.8, 5.4 Hz, 1H), 2.69 (td, J=5.5, 3.4 Hz, 2H), 2.25-2.08 (m, 2H), 1.92-1.77 (m, 2H), 1.50 (s, 9H), 1.32-1.25 (m, 5H). LCMS m/z 380.33 [M+H]⁺.

### Step 2. Synthesis of (2S,4R)-2′-(tert-butyl)-2-methyl-1-((1-methyl-1H-pyrazol-4-yl)methyl)-4′,5′-dihydrospiro[piperidine-4,7′-thieno[2,3-c]pyran] (1020)

To a solution of tert-butyl (2S,4R)-2′-(tert-butyl)-2-methyl-4′,5′-dihydrospiro[piperidine-4,7′-thieno[2,3-c]pyran]-1-carboxylate C101 (60 mg, 0.142 mmol) in dioxane (0.9 mL) was added 4 M HCl (395 μL, 1.580 mmol). The resulting solution was allowed to stir at room temperature overnight, then concentrated in vacuo. The crude material was dissolved in dichloromethane (2.5 mL), and to this solution were added 1-methylpyrazole-4-carbaldehyde (35 mg, 0.318 mmol), acetic acid (47 mg, 0.783 mmol), and polymer-supported cyanoborohydride (2 mmol/g) (236 mg, 0.472 mmol). The resulting solution was heated to 110° C. in a microwave reactor for 35 min, after which time the solution was cooled to room temperature. The solution was filtered and the solvent was removed in vacuo. The crude product was purified by reversed-phase HPLC: ACN in water (TFA modifier) C18 column, followed by lyophilization to afford (2S,4R)-2′-(tert-butyl)-2-methyl-1-((1-methyl-1H-pyrazol-4-yl)methyl)-4′,5′-dihydrospiro[piperidine-4,7′-thieno[2,3-c]pyran](Trifluoroacetate salt) (15.3 mg, 18%). ¹H NMR (300 MHz, Chloroform-d) δ 11.92 (s, 1H), 7.66 (s, 1H), 7.53 (d, J=0.8 Hz, 1H), 6.48 (s, 1H), 4.51 (d, J=14.2 Hz, 1H), 4.04 (dd, J=14.1, 4.0 Hz, 1H), 3.97 (s, 3H), 3.84 (q, J=5.8 Hz, 2H), 3.34 (d, J=12.1 Hz, 2H), 3.08 (t, J=11.4 Hz, 1H), 2.62 (q, J=5.1 Hz, 2H), 2.40-2.27 (m, 2H), 2.20-2.08 (m, 2H), 1.58 (d, J=6.5 Hz, 3H), 1.35 (s, 9H). LCMS m/z 374.2 [M+H]⁺.

### Preparation 1021

### (2S)-2′-chloro-2-methyl-1-((1-methyl-1H-pyrazol-4-yl)methyl)-5a′,6′,7′,8′,9′,9a′-hexahydrospiro[piperidine-4,4′-thieno[2,3-c]chromene](1021)

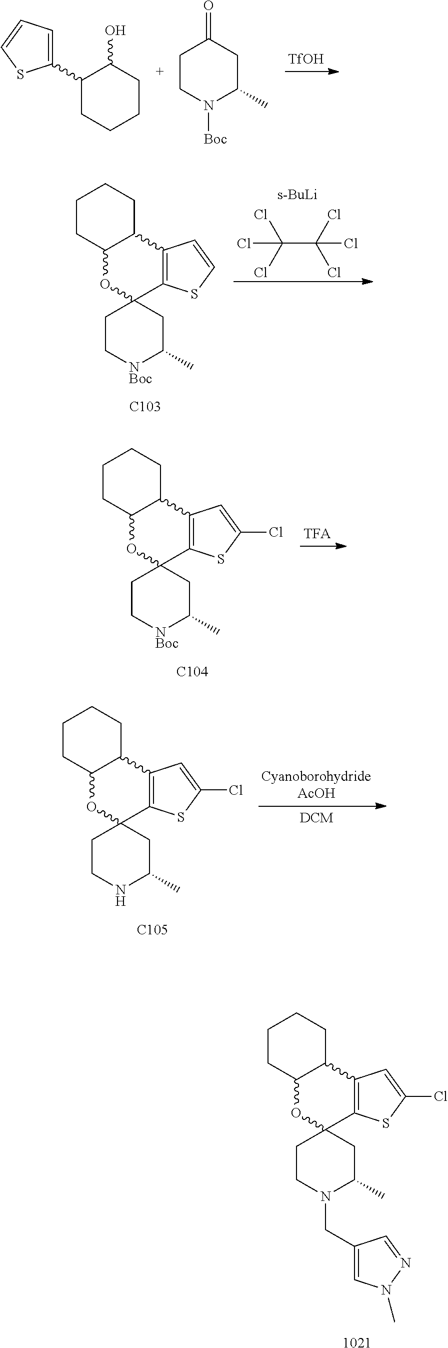

### Step 1. Synthesis of tert-butyl (2S)-2-methyl-5a′,6′,7′,8′,9′,9a′-hexahydrospiro[piperidine-4,4′-thieno[2,3-c]chromene]-1-carboxylate (C103)

To a solution of tert-butyl (S)-2-methyl-4-oxopiperidine-1-carboxylate (335 mg, 1.571 mmol) and 2-(3-thienyl)cyclohexanol (270 mg, 1.481 mmol) in DCM (1.8 mL) was added TFA (1.8 mL). The resulting solution was allowed to stir for 30 min, then concentrated in vacuo. The residue was dissolved in dioxane (4.5 mL) and cooled to 0° C. To this solution was added trifluoromethanesulfonic acid (680.00 mg, 0.4 mL, 4.531 mmol) at dropwise over 5 minutes, and the solution was allowed to warm to room temperature and stirred for 3 h. The reaction was quenched with aqueous NaOH solution (1N, 50 mL) then diluted with EtOAc (50 mL). The aqueous layer was separated, and further extracted with EtOAc (2×300 mL). The combined organic layers were dried over anhydrous sodium sulfate, then filtered and concentrated. The residue was dissolved in DCM (2.5 mL), and to this solution was added DIEA (408 mg, 0.55 mL, 3.158 mmol) and boc anhydride (360 mg, 1.650 mmol). The resulting solution was stirred at room temperature overnight, then diluted with DCM (25 mL) and sequentially washed with water and brine. The organic layer was dried over sodium sulfate then filtered and concentrated to afford tert-butyl (2S)-2-methyl-5a′,6′,7′,8′,9′,9a′-hexahydrospiro[piperidine-4,4′-thieno[2,3-c]chromene]-1-carboxylate. The crude product was used without further purification.

### Step 2. Synthesis of (2S)-2′-chloro-2-methyl-5a′,6′,7′,8′,9′,9a′-hexahydrospiro[piperidine-4,4′-thieno[2,3-c]chromene](C105)

To a solution of tert-butyl (2S)-2-methyl-5a′,6′,7′,8′,9′,9a′-hexahydrospiro[piperidine-4,4′-thieno[2,3-c]chromene]-1-carboxylate C103 (535 mg, 1.417 mmol) in THF (7.0 mL) at −78° C. was added sec-BuLi (2.0 mL of 1.4 M, 2.800 mmol) dropwise over 5 min. The resulting solution was stirred at that same temperature for 1 h, then a solution of 1,1,1,2,2,2-hexachloroethane (500 mg, 2.112 mmol) in THF (3.0 mL) was added dropwise. The reaction mixture was stirred at −78° C. for 1 h, then quenched with saturated ammonium chloride solution (5 mL) and diluted with ethyl acetate (50 mL) and water (50 mL). The organic layer was separated and dried over sodium sulfate, then filtered and concentrated to afford tert-butyl (2R)-2′-chloro-2-methyl-5a′,6′,7′,8′,9′,9a′-hexahydrospiro[piperidine-4,4′-thieno[2,3-c]chromene]-1-carboxylate, which was used without further purification. To a solution of tert-butyl (2R)-2′-chloro-2-methyl-5a′,6′,7′,8′,9′,9a′-hexahydrospiro[piperidine-4,4′-thieno[2,3-c]chromene]-1-carboxylate (50 mg, 0.073 mmol) in DCM (0.5 mL) was added TFA (29.6 mg, 0.02 mL, 0.260 mmol), and the resulting solution was stirred at room temperature for 2 h. The reaction mixture was concentrated, and the crude product was purified by reverse phase HPLC to afford (2S)-2′-chloro-2-methyl-5a′,6′,7′,8′,9′,9a′-hexahydrospiro[piperidine-4,4′-thieno[2,3-c]chromene](4.2 mg, 14%) as a white powder. LCMS m/z 312.3 [M+H]⁺.

### Step 3. Synthesis of (2S)-2′-chloro-2-methyl-1-((1-methyl-1H-pyrazol-4-yl)methyl)-5a′,6′,7′,8′,9′,9a′-hexahydrospiro[piperidine-4,4′-thieno[2,3-c]chromene](1021)

To a solution of (2S)-2′-(tert-butyl)-2-methyl-5a′,6′,7′,8′,9′,9a′-hexahydrospiro[piperidine-4,4′-thieno[2,3-c]chromene](C105) (62 mg, 0.199 mmol) and 1-methyl-1H-pyrazole-4-carbaldehyde (28 mg, 0.254 mmol) in dichloromethane (1.0 mL) was added acetic acid (53 mg, 0.879 mmol) and polymer-supported cyanoborohydride (2 mmol/g) (250 mg, 0.500 mmol). The resulting solution was heated to 110° C. in a microwave reactor for 20 minutes, after which time the solution was cooled to room temperature. The solution was filtered and the solvent was removed in vacuo. The crude product was purified by reversed-phase HPLC: ACN in water (TFA modifier) C18 column, followed by lyophilization to afford ((2S)-2′-chloro-2-methyl-1-((1-methyl-1H-pyrazol-4-yl)methyl)-5a′,6′,7′,8′,9′,9a′-hexahydrospiro[piperidine-4,4′-thieno[2,3-c]chromene](1021) (Trifluoroacetate salt) (8.9 mg, 11%) as a white powder. The product was isolated as a mixture of diastereomers with unknown absolute stereochemistry. ¹H NMR (500 MHz, DMSO-d6) δ 9.61 (s, 1H), 7.92 (s, 1H), 7.60 (s, 1H), 7.01 (d, J=1.8 Hz, 1H), 4.48-4.39 (m, 1H), 4.22-4.14 (m, 1H), 3.87 (s, 3H), 3.32-3.21 (m, 2H), 3.16-3.03 (m, 1H), 2.30-2.19 (m, 2H), 2.11-1.89 (m, 2H), 1.89-1.66 (m, 4H), 1.46-1.40 (m, 3H), 1.33 (q, J=11.4, 11.4, 11.2 Hz, 3H), 1.17-1.02 (m, 1H). Additional protons concealed beneath water peak. LCMS m/z 405.3 [M+H]⁺.

### Preparation of S80

### (1-(thiophen-3-yl)cyclopropyl)methanol (S80)

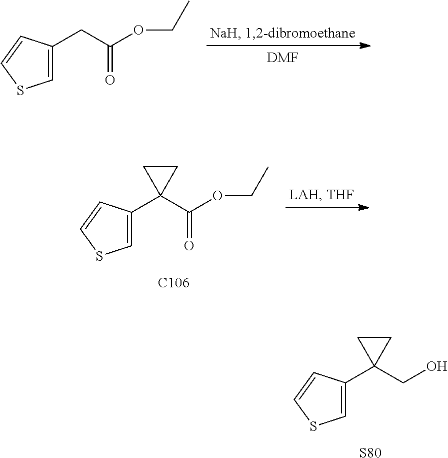

### Step 1. Synthesis of ethyl 1-(thiophen-3-yl)cyclopropane-1-carboxylate (C106)

Ethyl 2-(3-thienyl)acetate (2.84 g, 2.5 mL, 16.70 mmol) and 1,2-dibromoethane (6.97 g, 3.2 mL, 37.13 mmol) were dissolved in DMF (80 mL) then NaH (1.9 g, 60% w/w, 47.50 mmol) was added and the mixture stirred at 0° C. for 30 minutes then allowed to warm up to room temperature overnight. The reaction was then cooled to 0° C. and additional 1,2-dibromoethane (4.36 g, 2 mL, 23.21 mmol) followed by NaH (950 mg, 60% w/w, 23.752 mmol) were added. The mixture stirred at 0° C. for 30 min and was then warmed to room temperature. After stirring for 16 hours, the reaction was quenched by the addition of 1M HCl (100 mL) and diluted with water (400 mL). The aqueous layer was extracted three times with EtOAc (75 mL) and the organic layer was washed 5 times with brine (5×50 mL). The organic layer was dried over Na₂SO₄ and concentrated to provide ethyl 1-(3-thienyl)cyclopropanecarboxylate (2.0 g, 46%) as a colorless oil which was carried forward without further purification.

### Step 2. Synthesis of (1-(thiophen-3-yl)cyclopropyl)methanol (S80)

Ethyl 1-(3-thienyl)cyclopropanecarboxylate (3.27 g, 16.69 mmol) was dissolved in THF (65 mL) cooled to 0° C., then lithium aluminum hydride (9.15 mL of 2 M, 18.30 mmol) was added and the reaction was allowed to warm to stir at 0° C. for 40 minutes. Then, 50 mL of Et2O were added. 0.5 mL of DI water was added, followed by 0.5 mL of 15% NaOH, then 1.5 mL of DI water were added slowly (Fieser workup). The solids were filtered off and filtrate was dry loaded on to silica gel and purified by flash column chromatography using 0-30% ethyl acetate in hexanes to give [1-(3-thienyl)cyclopropyl]methanol (500 mg, 17%) as a colorless oil. ¹H NMR (250 MHz, CDCl3) δ 7.35-7.22 (m, 1H), 7.21-7.08 (m, 1H), 7.08-6.93 (m, 1H), 3.71 (s, 2H), 0.89 (d, J=8.4 Hz, 4H).

### Preparation 1022

### (2″S)-2′-chloro-2″-methyl-1″-((1-(2-(methylsulfonyl)ethyl)-1H-pyrazol-4-yl)methyl)-5′H-dispiro[cyclopropane-1,4′-thieno[2,3-c]pyran-7′,4″-piperidine](1022)

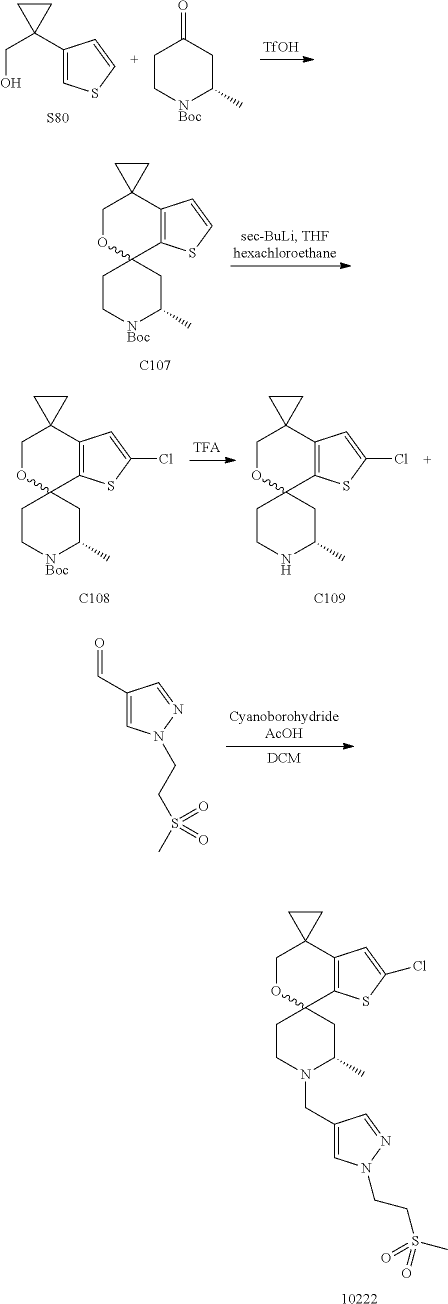

### Step 1. Synthesis of tert-butyl (2″S)-2″-methyl-5′H-dispiro[cyclopropane-1,4′-thieno[2,3-c]pyran-7′,4″-piperidine]-1″-carboxylate (C107)

[1-(3-thienyl)cyclopropyl]methanol S80 (150 mg, 0.9726 mmol) was dissolved in DCM (1 mL), then tert-butyl (2S)-2-methyl-4-oxo-piperidine-1-carboxylate (245 mg, 1.1488 mmol) and TFA (1 mL) were added and the reaction was allowed to stir for 20 min at room temperature. The volatiles were removed, and the crude residue was dissolved in dioxane (2 mL) and cooled to 0° C. TfOH (424.00 mg, 0.25 mL, 2.8252 mmol) was added and the reaction was allowed to stir at room temperature for 3 hours. The reaction was quenched by the addition of 1M NaOH (5 mL). The aqueous layer was extracted with EtOAc three times (3×5 mL). The organic layer was dried over Na₂SO₄ and concentrated. The crude residue was dissolved in DCM (6 mL) then Boc₂O (237 mg, 1.0859 mmol) and DIEA (259.70 mg, 0.35 mL, 2.0094 mmol) were added and the reaction allowed to stir overnight. The crude reaction mixture was directly loaded on to silica gel and purified by flash column chromatography using 0-30% EtOAc in hexanes to give tert-butyl (2″S)-2″-methyl-5′H-dispiro[cyclopropane-1,4′-thieno[2,3-c]pyran-7′,4″-piperidine]-1″-carboxylate (255 mg, 68%) as colorless crystals. ESI-MS m/z calc. 349.1712, found 350.2 (M+1)⁺.

### Step 2. Synthesis of tert-butyl 2′-chloro-5′H-dispiro[cyclopropane-1,4′-thieno[2,3-c]pyran-7′,4″-piperidine]-1″-carboxylate (C108)

tert-butyl (2″S)-2″-methyl-5′H-dispiro[cyclopropane-1,4′-thieno[2,3-c]pyran-7′,4″-piperidine]-1″-carboxylate (110 mg, 0.327 mmol) was dissolved in THF (1.5 mL) and cooled to −78° C. then sec-buli (0.5 mL of 1.4 M, 0.7000 mmol) was added dropwise. The reaction was stirred at that temperature for 1 hour, then 1,1,1,2,2,2-hexachloroethane (155 mg, 0.6547 mmol) in THF (0.6 mL) was added and the reaction was stirred for 30 min. The reaction was quenched by the addition of NH₄Cl (2 mL). The reaction was diluted with water and EtOAc (5 mL each) then the aqueous layer was extracted twice with EtOAc (2×5 mL). The organic layer was dried over Na₂SO₄ and concentrated. The crude material was purified by reverse phase HPLC purification to give C18H24ClNO3S (28.6 mg, 22%) as a colorless white solid. ESI-MS m/z calc. 369.1165, found 370.3 (M+1)⁺.

### Step 3. Synthesis of 2′-chloro-5′H-dispiro[cyclopropane-1,4′-thieno[2,3-c]pyran-7′,4″-piperidine](C109)

To a solution of tert-butyl 2′-chloro-5′H-dispiro[cyclopropane-1,4′-thieno[2,3-c]pyran-7′,4″-piperidine]-1″-carboxylate C108 (28.6 mg, 0.0773 mmol) in DCM (0.5 mL) was added with TFA (148.00 mg, 0.1 mL, 1.298 mmol) and stirred for 2 hours at room temperature. Volatiles were removed under reduced pressure to afford the crude product, 2′-chloro-5′H-dispiro[cyclopropane-1,4′-thieno[2,3-c]pyran-7′,4″-piperidine]29 mg of the crude product was used in the next step with out further purification. ESI-MS m/z calc. 269.0641, found 270.3 (M+1)⁺.

### Step 4. Synthesis of (2″R)-2′-chloro-2″-methyl-1″-((1-(2-(methylsulfonyl)ethyl)-1H-pyrazol-4-yl)methyl)-5′H-dispiro[cyclopropane-1,4′-thieno[2,3-c]pyran-7′,4″-piperidine](1022)

To a solution of (2″S)-2′-chloro-2″-methyl-5′H-dispiro[cyclopropane-1,4′-thieno[2,3-c]pyran-7′,4″-piperidine] C109 (81.6 mg, 0.213 mmol) in DCM (5 mL) was added TFA (1.48 g, 1 mL, 12.98 mmol) and the resulting solution was stirred at room temperature for 1 h. The reaction mixture was concentrated in vacuo for 1 h, then the residue was dissolved in DCM (3 mL). To the resulting solution were added acetic acid (84.48 mg, 0.08 mL, 1.407 mmol), 1-(2-methylsulfonylethyl)pyrazole-4-carbaldehyde (80 mg, 0.396 mmol), and polymer supported cyanoborohydride (182 mg, 2 mmol/g, 0.364 mmol). The resulting solution was heated to 110° C. in a microwave reactor for 30 min, after which time the solution was cooled to room temperature. The solution was filtered, and the solvent was removed in vacuo. The crude product was purified by reversed-phase HPLC: ACN in water (TFA modifier) C18 column, followed by lyophilization to afford (2″R)-2′-chloro-2″-methyl-1″-((1-(2-(methylsulfonyl)ethyl)-1H-pyrazol-4-yl)methyl)-5′H-dispiro[cyclopropane-1,4′-thieno[2,3-c]pyran-7′,4″-piperidine](1022) (25.3 mg, 17%) (trifluoroacetate salt) as a yellow gel. ¹H NMR (500 MHz, DMSO-d₆) δ 9.61 (s, 1H), 7.92 (s, 1H), 7.60 (s, 1H), 7.01 (d, J=1.8 Hz, 1H), 4.48-4.39 (m, 1H), 4.22-4.14 (m, 1H), 3.87 (s, 3H), 3.32-3.21 (m, 2H), 3.16-3.03 (m, 1H), 2.30-2.19 (m, 2H), 2.11-1.89 (m, 2H), 1.89-1.66 (m, 4H), 1.46-1.40 (m, 3H), 1.33 (q, J=11.4, 11.4, 11.2 Hz, 3H), 1.17-1.02 (m, 1H). Some protons under water peak. LCMS m/z 470.5 [M+H]⁺.

### Preparation 1023

### (2′S)-2′-methyl-1′-((1-(2-(methylsulfonyl)ethyl)-1H-pyrazol-4-yl)methyl)-3,4-dihydrospiro[benzo[4,5]thieno[2,3-c]pyran-1,4′-piperidine] (1023)

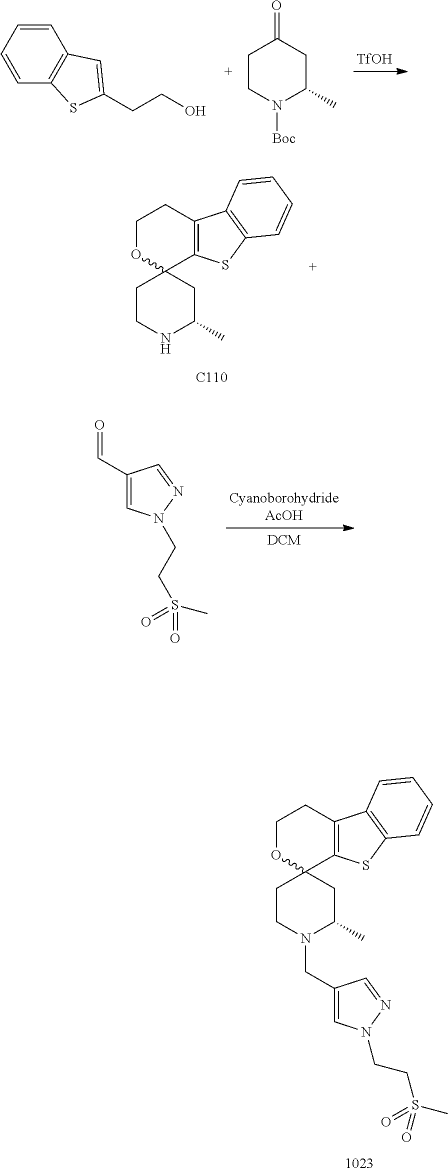

### Step 1. Synthesis of (1R,2′S)-2′-methyl-3,4-dihydrospiro[benzo[4,5]thieno[2,3-c]pyran-1,4′-piperidine] (C110)

A solution of tert-butyl (2S)-2-methyl-4-oxo-piperidine-1-carboxylate (298 mg, 1.40 mmol) and 2-(benzothiophen-3-yl)ethanol (300 mg, 1.683 mmol) in dioxane (4 mL) was cooled to 0° C. To the solution was added triflic acid (376 μL, 4.25 mmol) dropwise. The resulting solution was allowed to warm to room temperature and stirred for 3 h, after which time the reaction was diluted with DCM followed by 2M aqueous sodium carbonate solution. The organic layer was separated, and the aqueous layer was extracted with DCM. The combined organic layers were dried over anhydrous sodium sulfate, then filtered and concentrated. The crude product was purified by reversed-phase HPLC: 5-95% ACN in water (TFA modifier) C18 column, followed by lyophilization to afford (2′S,4R)-2-ethyl-2′-methyl-spiro[6,7-dihydrothieno[3,2-c]pyran-4,4′-piperidine] (C110) (TFA salt) as a white solid (412.5 mg, 76%). ¹H NMR (300 MHz, Methanol-d₄) δ 7.90-7.79 (m, 1H), 7.79-7.62 (m, 1H), 7.45-7.26 (m, 2H), 4.12 (qd, J=5.8, 2.2 Hz, 2H), 3.87-3.33 (m, 3H), 2.88 (td, J=5.5, 1.0 Hz, 2H), 2.39-1.87 (m, 4H), 1.56 (d, J=7.0 Hz, 1H), 1.35 (d, J=6.6 Hz, 2H). LCMS m/z 274.19 [M+H]⁺.

### Step 2. (2′S)-2′-methyl-1′-((1-(2-(methylsulfonyl)ethyl)-1H-pyrazol-4-yl)methyl)-3,4-dihydrospiro[benzo[4,5]thieno[2,3-c]pyran-1,4′-piperidine] (1023)

To a solution of (2′S)-2′-methylspiro[3,4-dihydrobenzothiopheno[2,3-c]pyran-1,4′-piperidine] (Trifluoroacetate salt) (C110) (88 mg, 0.227 mmol) and 1-(2-methylsulfonylethyl)pyrazole-4-carbaldehyde (68.5 mg, 0.339 mmol) in dichloromethane (2.4 mL) was added acetic acid (68 mg, 1.13 mmol) and polymer-supported cyanoborohydride (2 mmol/g) (340 mg, 0.680 mmol). The resulting solution was heated to 110° C. in a microwave reactor for 45 min, after which time the solution was cooled to room temperature. The solution was filtered and the solvent was removed in vacuo. The crude product was purified by reversed-phase HPLC: ACN in water (TFA modifier) C18 column, followed by lyophilization to afford (2′S)-2′-methyl-1′-[[1-(2-methylsulfonylethyl)pyrazol-4-yl]methyl]spiro[3,4-dihydrobenzothiopheno[2,3-c]pyran-1,4′-piperidine](Trifluoroacetate salt) (37 mg, 28%) The product was isolated as a mixture of diastereomers with unknown absolute stereochemistry. ¹H NMR (500 MHz, DMSO-d₆) δ 9.57 (s, 1H), 8.05 (d, J=40.9 Hz, 1H), 7.70 (d, J=27.5 Hz, 1H), 6.73 (d, J=19.0 Hz, 1H), 4.64-4.55 (m, 2H), 4.20 (d, J=5.4 Hz, 2H), 3.85 (d, J=12.0 Hz, 1H), 3.70 (t, J=6.8, 6.8 Hz, 2H), 3.65-3.54 (m, 1H), 3.41-3.29 (m, 1H), 3.21 (d, J=12.9 Hz, 1H), 2.92 (s, 3H), 2.39 (d, J=15.6 Hz, 1H), 2.31-2.19 (m, 1H), 2.11-1.99 (m, 2H), 1.52-1.39 (m, 3H), 1.02-0.92 (m, 1H), 0.92-0.82 (m, 2H). Additional protons under water peak. LCMS m/z 470.5 [M+H]⁺.

### Preparation S81

### (2′S, 7R)-2,2′-dimethylspiro[4,5-dihydrothieno[2,3-c]pyran-7,4′-piperidine] (S81)

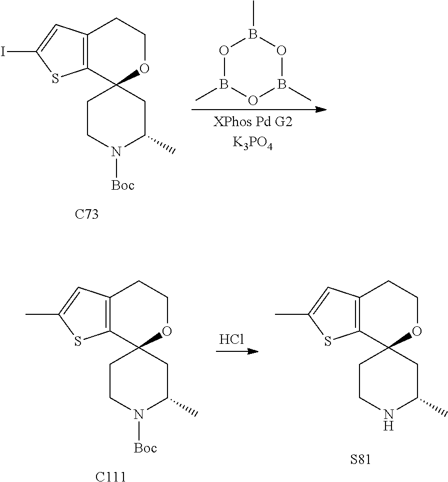

### Step 1. Synthesis of tert-butyl (2′S, 7R)-2,2′-dimethylspiro[4,5-dihydrothieno[2,3-c]pyran-7,4′-piperidine]-1′-carboxylate (C111)

To a 20 mL scintillation vial was added tert-butyl (2′S,7R)-2-iodo-2′-methyl-spiro[4,5-dihydrothieno[2,3-c]pyran-7,4′-piperidine]-1′-carboxylate C73 (1.19 g, 2.648 mmol), K₃PO₄ (1.78 g, 8.386 mmol), XPhos Pd G2 (113 mg, 0.1436 mmol), and the vial was purged with N₂ for 15 min. Then dioxane (8.8 mL) and 2,4,6-trimethyl-1,3,5,2,4,6-trioxatriborinane (1.1 mL, 7.869 mmol) were added. The reaction mixture was stirred at 80° C. After 4 h, the reaction was cooled to rt and washed with water and extracted with EtOAc (3×10 mL). The combined organic layer was dried over Na₂SO₄ and filtered through a plug of Celite®. The crude material was purified with a 150 g HP diol column and eluted with 0 to 10% EtOAc in heptane to provide tert-butyl (2′S,7R)-2,2′-dimethylspiro[4,5-dihydrothieno[2,3-c]pyran-7,4′-piperidine]-1′-carboxylate (421 mg, 44%). ¹H NMR (300 MHz, Chloroform-d₃) δ 6.41 (s, 1H), 4.00 (dt, J=11.6, 5.9 Hz, 1H), 3.87 (t, J=5.5 Hz, 2H), 3.73 (dt, J=13.7, 5.5 Hz, 1H), 3.35 (ddd, J=14.0, 8.7, 5.4 Hz, 1H), 2.59 (q, J=5.1, 4.5 Hz, 2H), 2.44 (d, J=1.1 Hz, 3H), 2.30-2.04 (m, 2H), 1.96-1.68 (m, 2H), 1.50 (s, 9H), 1.28 (d, J=6.6 Hz, 3H).

### Step 2. Synthesis of (2′S, 7R)-2,2′-dimethylspiro[4,5-dihydrothieno[2,3-c]pyran-7,4′-piperidine] (S81)

To a 50 mL flask was added tert-butyl (2′S,7R)-2,2′-dimethylspiro[4,5-dihydrothieno[2,3-c]pyran-7,4′-piperidine]-1′-carboxylate C111 (494 mg, 1.464 mmol) and dioxane (6 mL) at rt. Then HCl (1.8 mL of 4 M in dioxane, 7.200 mmol) was added. After 18 h, the reaction was quenched with saturated NaHCO₃ solution and extracted with EtOAc (×4). The combined organic layer was dried over Na₂SO₄, filtered, and concentrated. The crude material was purified by silica gel column chromatography (Gradient: 0 to 20% MeOH in DCM) to provide (2′S,7R)-2,2′-dimethylspiro[4,5-dihydrothieno[2,3-c]pyran-7,4′-piperidine](291 mg, 69%). LCMS m/z 238.11 [M+H]⁺.

### Compound 1024

### (2′S, 7R)-2,2′-dimethyl-1′-(1H-pyrazol-4-ylmethyl)spiro[4,5-dihydrothieno[2,3-c]pyran-7,4′-piperidine] (1024)

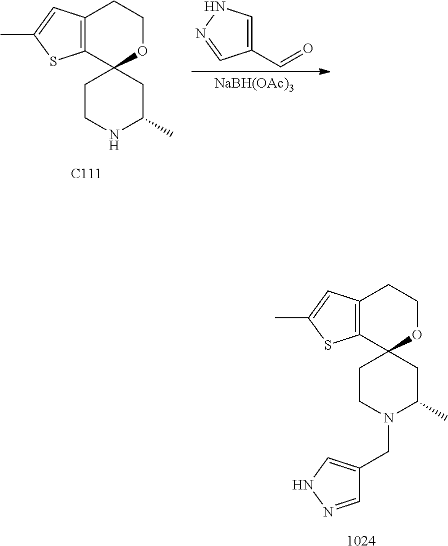

Standard Method: Reductive Amination with Sodium Triacetoxyborohydride

To a 1-dram vial was added (2′S,7R)-2,2′-dimethylspiro[4,5-dihydrothieno[2,3-c]pyran-7,4′-piperidine]C111 (30 mg, 0.1037 mmol), 1H-pyrazole-4-carbaldehyde (20 mg, 0.2081 mmol), followed by DCM (500 μL). Then sodium triacetoxyborohydride (66 mg, 0.3129 mmol) was added last. After 16 h at room temperature, the reaction mixture was quenched with saturated NaHCO₃ solution and extracted with EtOAc (×8). The combined organic layer was dried over MgSO₄, filtered, and concentrated in vacuo. Purification by reversed-phase HPLC. Method: C18 Waters Sunfire column (30×150 mm, 5 micron). Gradient: MeCN in H₂O with 0.2% formic acid. Product was isolated as (2′S,7R)-2,2′-dimethyl-1′-(1H-pyrazol-4-ylmethyl)spiro[4,5-dihydrothieno[2,3-c]pyran-7,4′-piperidine] (17.4 mg, 51%). LCMS m/z 318.21 [M+H]⁺. ¹H NMR (300 MHz, Chloroform-d) δ 7.54 (s, 2H), 6.39 (s, 1H), 4.01-3.72 (m, 3H), 3.61 (d, J=14.1 Hz, 1H), 2.88-2.46 (m, 6H), 2.41 (s, 3H), 1.88 (s, 3H), 1.71 (t, J=12.8 Hz, 1H), 1.22 (d, J 6.2 Hz, 3H).

### Compounds 1025-1026

Compounds 1025-1026 (see Table 48) were prepared in a single step from intermediate C111 using standard method as described in the preparation of compound 1024. Aldehydes were obtained from commercial sources or described previously. Any modifications to methods are noted in Table 48 and accompanying footnotes.

TABLE 48
Structure and physicochemical data for compounds 1025-1026

| | | Aldehyde | ¹H NMR; LCMS m/z |
|---|---|---|---|
| Cmpd | Structure | Reagent | [M + H]⁺ |
| 1025 | 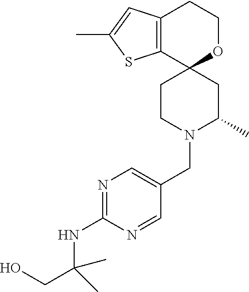 [SMILES: Cc1cc2c(s1)[C@]1(CCN(Cc3cnc(NC(C)(C)CO)nc3)[C@@H](C)C1)OCC2] | 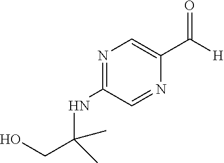 | ¹H NMR (300 MHz, Chloroform-d₃) δ 8.21 (s, 2H), 6.52 (s, 1H), 6.40 (d, J = 1.2 Hz, 1H), 5.21 (s, 1H), 3.99- 3.80 (m, 3H), 3.67 (s, 2H), 3.04 (d, J = 13.6 Hz, 1H), 2.59 (q, J = 5.9, 5.3 Hz, 4H), 2.42 (d, J = 1.1 Hz, 3H), 2.35 (d, J = 11.9 Hz, 1H), 1.98 (d, J = 14.9 Hz, 2H), 1.89-1.73 (m, 1H), 1.73-1.60 (m, 1H), 1.36 (s, 6H), 1.18 (d, J = 6.1 Hz, 3H); LCMS m/z 417.23 [M + H]⁺. |
| 1026 | 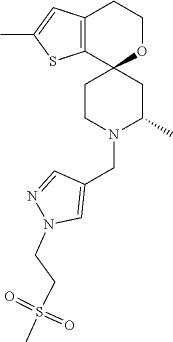 | 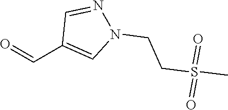 | ¹H NMR (300 MHz, Chloroform-d₃) δ 7.62 (s, 1H), 7.55 (s, 1H), 6.40 (s, 1H), 4.62 (t, J = 6.1 Hz, 2H), 4.11 (d, J = 14.4 Hz, 1H), 3.81 (dd, J = 10.0, 4.9 Hz, 3H), 3.64 (t, J = 6.2 Hz, 2H), 3.04-2.83 (m, 2H), 2.81-2.65 (m, 1H), 2.65- 2.56 (m, 2H), 2.54 (s, 3H), 2.41 (s, 3H), 2.14 (td, J = 13.6, 12.4, 4.6 Hz, 1H), 2.01 (d, J = 7.9 Hz, 3H), 1.36 (d, J = 6.4 Hz, 3H); LCMS m/z 424.11 [M + H]⁺. |

### Compound 1027

### 2-[4-[[(2′S, 7R)-2,2′-dimethylspiro[4,5-dihydrothieno[2,3-c]pyran-7,4′-piperidine]-1′-yl]methyl]pyrazol-1-yl]ethanol (1027)

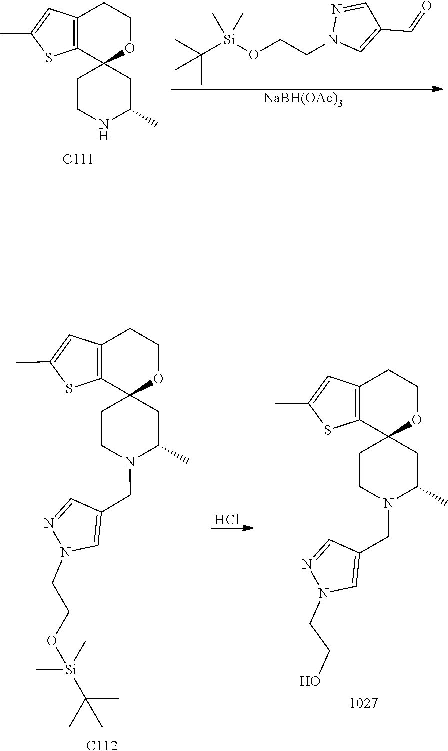

### Step 1. Synthesis of tert-butyl-[2-[4-[[(2′S, 7R)-2,2′-dimethylspiro[4,5-dihydrothieno[2,3-c]pyran-7,4′-piperidine]-1′-yl]methyl]pyrazol-1-yl]ethoxy]-dimethyl-silane (C112)

To a 1-dram vial was added (2′S,7R)-2,2′-dimethylspiro[4,5-dihydrothieno[2,3-c]pyran-7,4′-piperidine] C111 (30 mg, 0.1037 mmol), 1-[2-[tert-butyl(dimethyl)silyl]oxyethyl]pyrazole-4-carbaldehyde (66 mg, 0.2594 mmol) was added, followed by DCM (500 μL). Then sodium triacetoxyborohydride (77 mg, 0.3650 mmol) was added. After 19 hours, the reaction was quenched with saturated NaHCO₃ solution and extracted with EtOAc (×5). The combined organic layer was dried over Na₂SO₄, filtered, and concentrated in vacuo. The crude material was purified by silica gel chromatography (Gradient: 0 to 10% MeOH in DCM) to provide tert-butyl-[2-[4-[[(2′S,7R)-2,2′-dimethylspiro[4,5-dihydrothieno[2,3-c]pyran-7,4′-piperidine]-1′-yl]methyl]pyrazol-1-yl]ethoxy]-dimethyl-silane (64 mg, 114%). LCMS m/z 476.22 [M+H]⁺.

### Step 2. Synthesis of 2-[4-[[(2′S, 7R)-2,2′-dimethylspiro[4,5-dihydrothieno[2,3-c]pyran-7,4′-piperidine]-1′-yl]methyl]pyrazol-1-yl]ethanol (1027)

To the isolated product in step 1 C112 was added MeOH (600 μL), followed by HCl (44 μL of 11.7 M, 0.5148 mmol). After 30 min, the reaction mixture was concentrated and purified by reversed-phase HPLC. Method: C18 Waters Sunfire column (30×150 mm, 5 micron). Gradient: MeCN in H₂O with 0.1% trifluoroacetic acid. It was found that a portion of the product was converted to the corresponding trifluoroacetic ester. It was dissolved in DCM (1 mL), treated with 1N NaOH (1 mL). After 80 min, the mixture was separated, and the aqueous layer was extracted with DCM (×4). The combined organic layer was dried over Na₂SO₄, filtered, and concentrated in vacuo to provide 2-[4-[[(2′S,7R)-2,2′-dimethylspiro[4,5-dihydrothieno[2,3-c]pyran-7,4′-piperidine]-1′-yl]methyl]pyrazol-1-yl]ethanol (25.0 mg, 57%). ¹H NMR (300 MHz, Chloroform-d₃) δ 7.45 (s, 1H), 7.39 (s, 1H), 6.40 (d, J=1.2 Hz, 1H), 4.22 (dd, J=5.6, 3.9 Hz, 2H), 4.00 (dd, J=5.5, 4.1 Hz, 2H), 3.96-3.79 (m, 3H), 3.54 (d, J=14.1 Hz, 1H), 2.75 (d, J=11.9 Hz, 1H), 2.70-2.46 (m, 4H), 2.41 (d, J=1.2 Hz, 3H), 2.11-1.81 (m, 4H), 1.80-1.64 (m, 1H), 1.23 (t, J=6.2 Hz, 3H). LCMS m/z 362.09 [M+H]⁺.

### Compound 1028

### 2-[[4-[[(2′S, 7R)-2,2′-dimethylspiro[4,5-dihydrothieno[2,3-c]pyran-7,4′-piperidine]-1′-yl]methyl]pyrazol-1-yl]methyl]-2-methyl-propane-1,3-diol (1028)

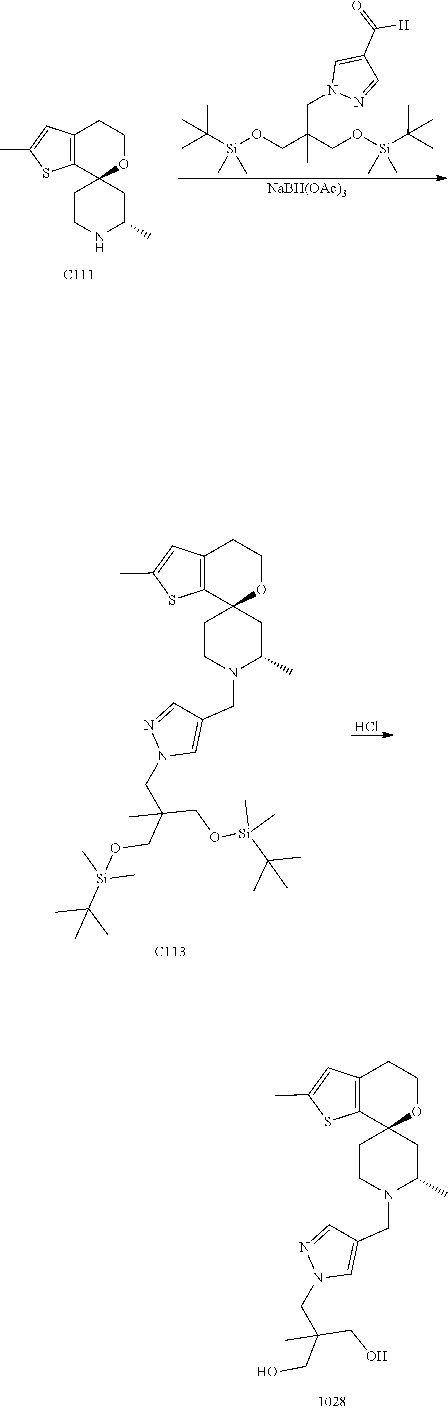

2-[[4-[[(2′S,7R)-2,2′-dimethylspiro[4,5-dihydrothieno[2,3-c]pyran-7,4′-piperidine]-1′-yl]methyl]pyrazol-1-yl]methyl]-2-methyl-propane-1,3-diol 1028 was prepared according to the procedure as in the preparation in compound 1027, except HCl (10 eq) was used in step 2. LCMS m/z 420.19 [M+H]⁺. ¹H NMR (300 MHz, Chloroform-d₃) δ 7.44 (s, 1H), 7.39 (s, 1H), 6.40 (d, J=1.3 Hz, 1H), 4.24 (d, J=2.1 Hz, 2H), 3.99-3.78 (m, 3H), 3.53 (td, J=12.0, 4.2 Hz, 4H), 3.34 (dd, J=11.3, 3.1 Hz, 2H), 2.78-2.46 (m, 5H), 2.41 (d, J=1.1 Hz, 3H), 2.09-1.80 (m, 4H), 1.72 (t, J=12.8 Hz, 1H), 1.21 (d, J=6.3 Hz, 3H), 0.82 (s, 3H).

### Compound 1029

### (2′S)-2,2′-dimethyl-1′-[[1-(2-methylsulfonylethyl)triazol-4-yl]methyl]spiro[4,5-dihydrothieno[2,3-c]pyran-7,4′-piperidine] (1029)

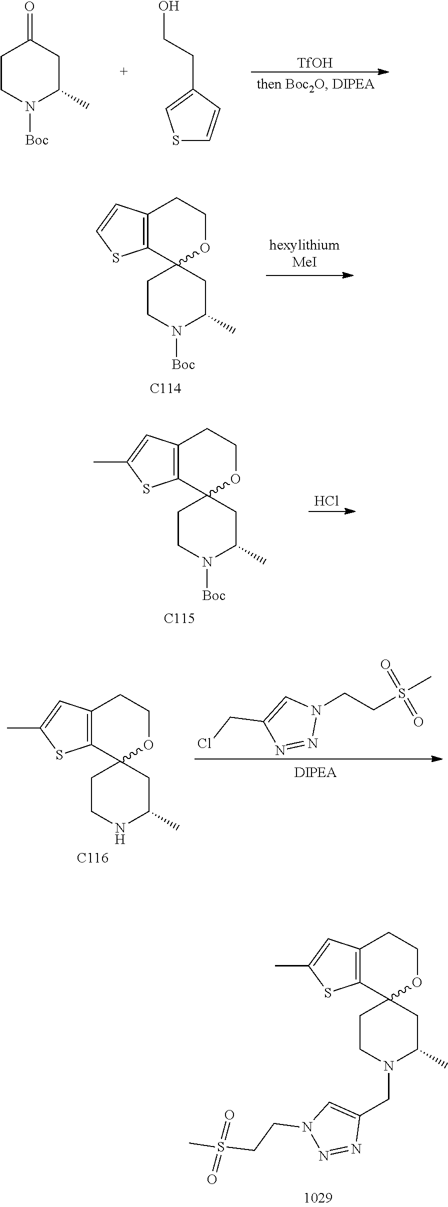

### Step 1. Synthesis of tert-butyl (2′S)-2′-methylspiro[4,5-dihydrothieno[2,3-c]pyran-7,4 piperidine]-1′-carboxylate (C114)

Tert-butyl (2S)-2-methyl-4-oxo-piperidine-1-carboxylate (332 mg, 1.557 mmol) was dissolved in dioxane (6 mL) and cooled in an ice bath and trifluoromethanesulfonic acid (721 mg, 4.804 mmol) was added. The reaction mixture was then stirred at rt for 15 min and 2-(3-thienyl)ethanol (200 mg, 1.560 mmol) added. The reaction mixture was stirred at room temperature for 3 h, and then diluted with 1N NaOH/EtOAc. The organic layer was washed with brine, dried and concentrated to an oil. The crude material was dissolved in DCM (9 mL). DIPEA (410 mg, 3.172 mmol) and Boc₂O (370 mg, 1.695 mmol) was added. After 2 h, the reaction mixture was concentrated in vacuo and purified by silica gel chromatography (Gradient: 0 to 20% EtOAc in hexanes) to provide tert-butyl (2′S)-2′-methylspiro[4,5-dihydrothieno[2,3-c]pyran-7,4′-piperidine]-1′-carboxylate (314 mg, 41%). LCMS m/z 323.27 [M+H]⁺.

### Step 2. Synthesis of tert-butyl (2′S, 7R)-2,2′-dimethylspiro[4,5-dihydrothieno[2,3-c]pyran-7,4′-piperidine]-1′-carboxylate (C115)

In a flask was dissolved tert-butyl (2′S)-2′-methylspiro[4,5-dihydrothieno[2,3-c]pyran-7,4′-piperidine]-1′-carboxylate C114 (100 mg, 0.3092 mmol) in THF (2.6 mL) and cooled to −78° C. To the reaction mixture was added hexyllithium (403 μL of 2.3 M, 0.9269 mmol) dropwise and the reaction mixture was stirred at −78° C. for 30 min. Then MeI (38.5 μL, 0.6184 mmol) was added and the reaction was warmed to rt and stirred overnight. The reaction was quenched with ammonium chloride solution and extracted with EtOAc, washed with brine and dried with sodium sulfate. The combined organic layer was concentrated in vacuo to give tert-butyl (2′S,7R)-2,2′-dimethylspiro[4,5-dihydrothieno[2,3-c]pyran-7,4′-piperidine]-1′-carboxylate (190 mg, 78%). LCMS m/z 338.15 [M+H]⁺.

### Step 3. Synthesis of (2′S)-2,2′-dimethylspiro[4,5-dihydrothieno[2,3-c]pyran-7,4′-piperidine](C116)

Tert-butyl (2′S)-2,2′-dimethylspiro[4,5-dihydrothieno[2,3-c]pyran-7,4′-piperidine]-1′-carboxylate C115 (190 mg, 0.2421 mmol) was dissolved in dioxane (3 mL) and HCl (605 μL of 4 M, 2.420 mmol) was added. The solution was stirred overnight. The solution was concentrated in vacuo to provide (2′S)-2,2′-dimethylspiro[4,5-dihydrothieno[2,3-c]pyran-7,4′-piperidine], which was used in the next step without further purification. LCMS m/z 238.09 [M+H]⁺.

### Step 4. Synthesis of (2′S)-2,2′-dimethyl-1′-[[1-(2-methylsulfonylethyl)triazol-4-yl]methyl]spiro[4,5-dihydrothieno[2,3-c]pyran-7,4′-piperidine](1029)

(2′S)-2,2′-dimethylspiro[4,5-dihydrothieno[2,3-c]pyran-7,4′-piperidine](57.47 mg, 0.24 mmol; prepared using unoptimized conditions and isolated as a mixture of diastereomers) was dissolved in DCE (3 mL) and DIPEA (42 μL, 0.2411 mmol) and 4-(chloromethyl)-1-(2-methylsulfonylethyl)triazole (54 mg, 0.2414 mmol) were added. The solution was stirred at 65° C. After 24 h, the solution as cooled and partitioned between DCM and 1N NaOH. The organic layer was collected through a phase separator tube then concentrated in vacuo. Purification by reversed-phase HPLC. Method: C18 Waters Sunfire column (30×150 mm, 5 micron). Gradient: MeCN in H₂O with 0.1% trifluoroacetic acid. Product was isolated as (2′S)-2,2′-dimethyl-1′-[[1-(2-methylsulfonylethyl)triazol-4-yl]methyl]spiro[4,5-dihydrothieno[2,3-c]pyran-7,4′-piperidine](Trifluoroacetate salt) (35.6 mg, 26%, 4:1 dr). LCMS m/z 425.19 [M+H]⁺. ¹H NMR (300 MHz, Methanol-d₄) δ 8.33 (s, 1H), 6.47 (d, J=1.4 Hz, 1H), 5.10-4.91 (m, 2H), 4.77-4.39 (m, 2H), 3.91-3.77 (m, 4H), 3.45-3.34 (m, 2H), 3.03-2.93 (m, 3H), 2.59 (q, J=5.8 Hz, 2H), 2.41 (d, J=1.2 Hz, 3H), 2.33-1.88 (m, 4H), 1.65 (d, J=7.1 Hz, 1H), 1.56 (d, J=6.4 Hz, 3H).

### Compound 1030

### (2′R, 7R)-2,2′-dimethyl-1′-[[1-(2-methylsulfonylethyl)pyrazol-4-yl]methyl]spiro[4,5-dihydrothieno[2,3-c]pyran-7,4′-piperidine](1030)

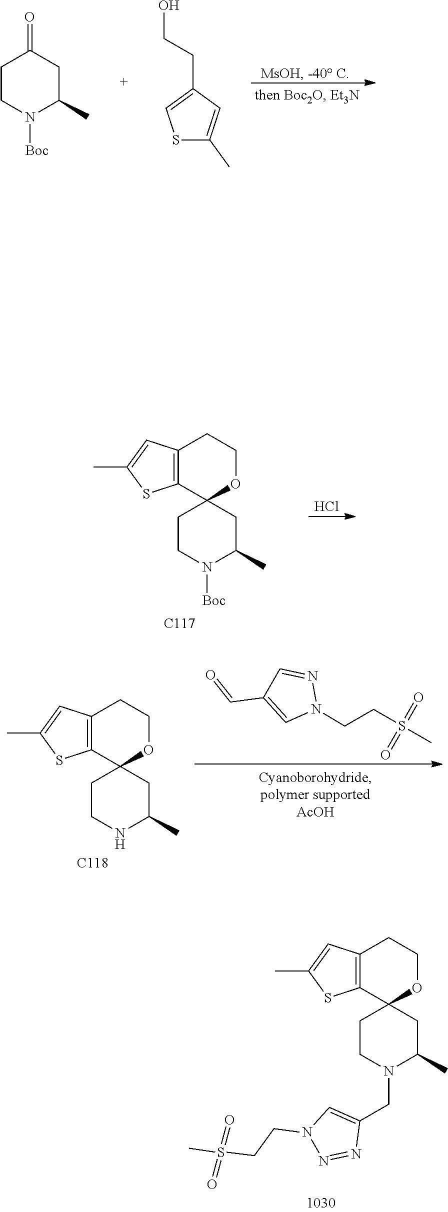

### Step 1. Synthesis of tert-butyl (2′R, 7R)-2,2′-dimethylspiro[4,5-dihydrothieno[2,3-c]pyran-7,4′-piperidine]-1′-carboxylate (C117)

A solution of tert-butyl (2R)-2-methyl-4-oxo-piperidine-1-carboxylate (252 mg, 1.182 mmol) and 2-(5-methyl-3-thienyl)ethanol (165 mg, 1.160 mmol) in DCM (1.5 mL) was cooled to −40° C. To the reaction was MsOH (220 μL, 3.390 mmol) added and the reaction was stirred at −40° C. for 2.5 h and allowed to warm to 0° C. The reaction mixture was quenched into a biphasic mixture of saturated NaHCO₃ solution (15 mL) and DCM (15 mL) and the aqueous layer pH was adjusted to >10 with 2N NaOH. The aqueous layer was then extracted with DCM (3×15 mL) and filtered through an SPE cartridge and concentrated to a lightly yellow gel. The crude material was dissolved in DCM (1.5 mL) and added Et₃N (330 μL, 2.368 mmol), Boc₂O (330 μL, 1.436 mmol). The reaction mixture was stirred at room temperature for 20 h. The solution was diluted with DCM and washed with water (10 mL). The aqueous layer was extracted with DCM (2×10 mL), dried over sodium sulfate, filtered through an SPE filter, and concentrated in vacuo. The resulting residue was purified by silica gel chromatography (Gradient: 0 to 30% EtOAc in heptane) to yield the two diastereomers at the spiro-center. The desired product was isolated as tert-butyl (2′R,7R)-2,2′-dimethylspiro[4,5-dihydrothieno[2,3-c]pyran-7,4′-piperidine]-1′-carboxylate (313.5 mg, 80%). 1H NMR (300 MHz, Methanol-d₄) δ 6.42 (d, J=1.2 Hz, 1H), 4.35 (p, J=7.0 Hz, 1H), 3.97-3.81 (m, 3H), 3.37-3.19 (m, 1H), 2.69-2.47 (m, 2H), 2.39 (d, J=1.1 Hz, 3H), 2.05 (dd, J=14.5, 2.1 Hz, 1H), 1.95 (dd, J=13.9, 2.6 Hz, 1H), 1.89-1.63 (m, 2H), 1.48 (s, 9H), 1.33 (d, J=7.1 Hz, 3H).

### Step 2. Synthesis of (2′R, 7R)-2,2′-dimethylspiro[4,5-dihydrothieno[2,3-c]pyran-7,4′-piperidine] (C118)

To a stirred solution of tert-butyl (2′R,7R)-2,2′-dimethylspiro[4,5-dihydrothieno[2,3-c]pyran-7,4′-piperidine]-1′-carboxylate C117 (205 mg, 0.6074 mmol) in DCM (2 mL) was added HCl (0.5 mL of 4 M in dioxane, 2.00 mmol) and the reaction was stirred for 3 h at room temperature. The reaction mixture was concentrated under a stream of nitrogen and subjected under reverse phase purification. Method: C18 Waters Sunfire column (30×150 mm, 5 micron). Gradient: MeCN in H₂O with 5 mM HCl. After concentration, the product was isolated (2′R,7R)-2,2′-dimethylspiro[4,5-dihydrothieno[2,3-c]pyran-7,4′-piperidine](Hydrochloride salt) (98.3 mg, 57%). LCMS m/z 238.0 [M+H]⁺. 1H NMR (300 MHz, Methanol-d₄) δ 6.52 (t, J=1.2 Hz, 1H), 3.95 (t, J=5.6 Hz, 2H), 3.72 (dq, J=11.7, 6.6 Hz, 1H), 3.52 (dt, J=13.2, 6.5 Hz, 1H), 3.28-3.19 (m, 1H), 2.62 (t, J=5.5 Hz, 2H), 2.43 (d, J=1.1 Hz, 3H), 2.24-1.99 (m, 4H), 1.50 (d, J=6.9 Hz, 3H).

### Step 3. Synthesis of (2′R, 7R)-2,2′-dimethyl-1′-[[1-(2-methylsulfonylethyl)pyrazol-4-yl]methyl]spiro[4,5-dihydrothieno[2,3-c]pyran-7,4′-piperidine](1030)

(2′R,7R)-2,2′-dimethylspiro[4,5-dihydrothieno[2,3-c]pyran-7,4′-piperidine](Hydrochloride salt) C118 (36 mg, 0.1315 mmol) and 1-(2-methylsulfonylethyl)pyrazole-4-carbaldehyde (28 mg, 0.1385 mmol) were dissolved in DCM (1 mL). Acetic acid (50 μL, 0.8792 mmol) was added to the solution, followed by cyanoborohydride, polymer-supported (252 mg of 2 mmol/g, 0.5040 mmol). The solution was capped and heated to 90° C. in a microwave reactor for 60 min. The suspension was stirred in MeOH (1.5 mL) for 10 min, before filtering off the resin (twice). The solvent was removed and the residue was dissolved into water (2 mL) and DCM (2 mL). The pH of the aqueous layer was adjusted with 2M NaOH to a pH >10. The phases were separated through a phase separator and the aqueous layer was extracted with DCM (2×10 mL) and the combined organics were concentrated in vacuo. The crude residue was purified by silica gel chromatography (Gradient: 0-20% MeOH in DCM) to yield (2′R,7R)-2,2′-dimethyl-1′-[[1-(2-methylsulfonylethyl)pyrazol-4-yl]methyl]spiro[4,5-dihydrothieno[2,3-c]pyran-7,4′-piperidine](55.5 mg, 95%). ¹H NMR (300 MHz, Methanol-d₄) δ 7.75 (s, 1H), 7.57 (d, J=0.8 Hz, 1H), 6.46 (d, J=1.1 Hz, 1H), 4.64 (t, J=6.6 Hz, 2H), 3.91 (t, J=5.6 Hz, 2H), 3.80-3.64 (m, 4H), 2.88 (dd, J=13.0, 6.5 Hz, 2H), 2.77 (d, J=0.8 Hz, 3H), 2.73-2.62 (m, 1H), 2.57 (t, J=5.6 Hz, 2H), 2.38 (d, J=1.1 Hz, 3H), 2.05-1.76 (m, 4H), 1.24 (d, J=6.4 Hz, 3H).

### Compound 1031

### (2′S, 7R)-4-(difluoromethyl)-2,2′-dimethyl-1′-[[1-(2-methylsulfonylethyl)pyrazol-4-yl]methyl]spiro[5H-thieno[2,3-c]pyran-7,4′-piperidine]-4-ol (1031)

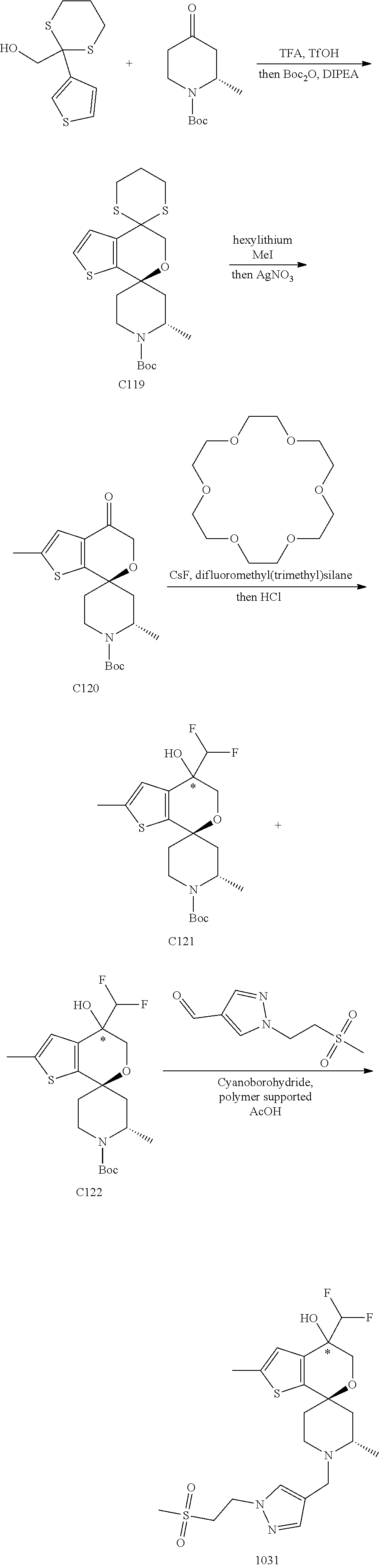

### Step 1. Synthesis of tert-butyl (6″S, 7′R)-6″-methyl-5′H-dispiro[1,3-dithiane-2,4′-thieno[2,3-c]pyran-7′,4″-piperidine]-1″-carboxylate (C119)

Tert-butyl (2S)-2-methyl-4-oxo-piperidine-1-carboxylate (524 mg, 2.457 mmol) was dissolved in DCM (21 mL) and TFA (1.13 mL, 14.67 mmol) was added and stirred for 30 min. The solvent was removed in vacuo. [2-(3-thienyl)-1,3-dithian-2-yl]methanol (600 mg, 2.453 mmol) was added into the same reaction flask, followed by dioxane (14 mL). The reaction mixture was cooled to 0° C. Triflic acid (434 μL, 4.905 mmol) was added dropwise while stirring vigorously and the reaction was warmed to rt. After 20 min, the reaction solution was diluted with DCM and washed with Na₂CO₃ and extracted with DCM (×3). The organic layer was collected, dried with sodium sulfate, filtered and the solvent was removed to give crude material: (6″S,7′R)-6″-methyl-5′H-dispiro[1,3-dithiane-2,4′-thieno[2,3-c]pyran-7′,4″-piperidine]

The crude material was dissolved in DCM (21 mL) and tert-butoxycarbonyl tert-butyl carbonate (733 μL, 3.191 mmol) and DIPEA (728 μL, 4.180 mmol) were added. The solution was stirred at rt for 4 h. The reaction was purified by silica gel column chromatography (Gradient: 0 to 50% EtOAc in hexanes) to give tert-butyl (6″S,7′R)-6″-methyl-5′H-dispiro[1,3-dithiane-2,4′-thieno[2,3-c]pyran-7′,4″-piperidine]-1″-carboxylate (250 mg, 20%). LCMS m/z 428.15 [M+H]⁺.

### Step 2. Synthesis of tert-butyl (2S,4R)-2,2′-dimethyl-4′-oxo-spiro[piperidine-4,7′-thieno[2,3-c]pyran]-1-carboxylate (C120)

To a solution of tert-butyl (6″S,7′R)-6″-methyl-5′H-dispiro[1,3-dithiane-2,4′-thieno[2,3-c]pyran-7′,4″-piperidine]-1″-carboxylate C119 (250 mg, 0.5846 mmol) in THF (10 mL) at −78° C. was added hexyllithium (838 μL of 2.3 M, 1.927 mmol) dropwise. The reaction was stirred for 1 h at −78° C., and MeI (36 μL, 0.5783 mmol) was added as a solution in THF. After stirring for 1 h, the reaction was quenched with NH₄Cl aqueous solution and partitioned between DCM and NH₄Cl solution. The aqueous layer was extracted with DCM (×3) and the combined organic layer was washed with brine, dried with sodium sulfate, and the solvent was removed in vacuo.

The crude product was dissolved in EtOH (10 mL) and silver nitrate (57 μL, 1.460 mmol) was added and heated to 50° C. After 2 h, the solution was partitioned between DCM and water, and the organic layer was collected through a phase separator. The solvent was removed in vacuo. Purification by silica gel chromatography (Gradient: 0-60% EtOAc in heptane) yielded the product tert-butyl (2S,4R)-2,2′-dimethyl-4′-oxo-spiro[piperidine-4,7′-thieno[2,3-c]pyran]-1-carboxylate (145 mg, 65%). LCMS m/z 352.42 [M+H]⁺. ¹H NMR (400 MHz, Methanol-d₄) δ 6.98 (q, J=1.2 Hz, 1H), 4.28 (s, 2H), 4.13-3.99 (m, 1H), 3.88-3.72 (m, 1H), 3.41-3.31 (m, 1H), 2.54-2.27 (m, 5H), 1.98-1.76 (m, 2H), 1.48 (s, 9H), 1.24 (d, J=6.5 Hz, 3H).

### Step 3. Synthesis of (2′S, 7R)-4-(difluoromethyl)-2,2′-dimethyl-spiro[5H-thieno[2,3-c]pyran-7,4′-piperidine]-4-ol (C121 and C122)

Under N₂, cesium fluoride (109 mg, 0.7176 mmol) and 1,4,7,10,13,16-hexaoxacyclooctadecane (189 mg, 0.7151 mmol) were added to a solution of tert-butyl (2S,4R)-2,2′-dimethyl-4′-oxo-spiro[piperidine-4,7′-thieno[2,3-c]pyran]-1-carboxylate C120 (250 mg, 0.7113 mmol) in 1,2-dimethoxyethane (6 mL). To the mixture was added difluoromethyl(trimethyl)silane (357 mg, 2.874 mmol) and the resulting mixture was stirred at room temperature. After stirring over 60 h, an additional difluoromethyl(trimethyl)silane (357 mg, 2.874 mmol) was added and the solution was stirred for 24 h. The reaction was quenched by partitioning between water and DCM, and the organics were collected through a phase separator tube and concentrated in vacuo to give the crude product.

It was dissolved in dioxane (2 mL) and HCl (2 mL of 4 M in dioxane, 8.000 mmol) was added. After 1 h, the solvent was removed in vacuo and the crude material was purified by reversed-phase HPLC. Method: C18 Waters Sunfire column (30×150 mm, 5 micron). Gradient: MeCN in H₂O with 0.2% formic acid. Two diastereomers were isolated after neutralizing with pH 10 buffer:

(2′S,7R)-4-(difluoromethyl)-2,2′-dimethyl-spiro[5H-thieno[2,3-c]pyran-7,4′-piperidine]-4-ol (33 mg, 30%). ¹H NMR (300 MHz, Methanol-d₄) δ 6.74 (t, J 1.3 Hz, 1H), 5.89 (t, J 55.4 Hz, 1H), 4.00 (d, J 11.9 Hz, 1H), 3.70 (ddd, J=12.0, 3.2, 1.7 Hz, 1H), 3.14-2.80 (m, 3H), 2.43 (d, J 1.2 Hz, 3H), 2.15 (dt, J=14.0, 2.6 Hz, 1H), 1.97-1.66 (m, 2H), 1.35 (dd, J=14.0, 11.5 Hz, 1H), 1.08 (d, J 6.4 Hz, 3H); and

(2′S,7R)-4-(difluoromethyl)-2,2′-dimethyl-spiro[5H-thieno[2,3-c]pyran-7,4′-piperidine]-4-ol (31 mg, 28%). ¹H NMR (300 MHz, Methanol-d₄) δ 6.74 (s, 1H), 5.89 (t, J 55.4 Hz, 1H), 4.01 (d, J 12.0 Hz, 1H), 3.74-3.60 (m, 1H), 3.15-2.80 (m, 3H), 2.43 (d, J 1.1 Hz, 3H), 2.12 (d, J 14.2 Hz, 1H), 1.94 (d, J 13.8 Hz, 1H), 1.78-1.57 (m, 1H), 1.53-1.36 (m, 1H), 1.08 (d, J 6.4 Hz, 3H).

### Step 4. Synthesis of (2′S, 7R)-4-(difluoromethyl)-2,2′-dimethyl-1′-[[1-(2-methylsulfonyl-ethyl)pyrazol-4-yl]methyl]spiro[5H-thieno[2,3-c]pyran-7,4′-piperidine]-4-ol (1031)

(2′S)-4-(difluoromethyl)-2,2′-dimethyl-spiro[5H-thieno[2,3-c]pyran-7,4′-piperidine]-4-ol C122 (20 mg, 0.06593 mmol) was added into a microwave vial and 1-(2-methylsulfonylethyl)pyrazole-4-carbaldehyde (26.67 mg, 0.1319 mmol), acetic acid (19 mg, 0.3164 mmol) and DCM (2 mL) were added. Cyanoborohydride, polymer-supported (98 mg of 2 mmol/g, 0.1960 mmol) was added. The reaction vial was heated to 95° C. in a microwave reactor for 2 h. MeOH was added and the solution was stirred for 10 min to wash the beads. The solution was filtered and the solvent removed in vacuo. Purification by silica gel chromatography (Gradient: 0-20% MeOH in DCM) yielded (2′S,7R)-4-(difluoromethyl)-2,2′-dimethyl-1′-[[1-(2-methylsulfonylethyl)pyrazol-4-yl]methyl]spiro[5H-thieno[2,3-c]pyran-7,4′-piperidine]-4-ol (8.2 mg, 25%). LCMS m/z 490.18 [M+H]⁺. ¹H NMR (300 MHz, Methanol-d₄) δ 7.71 (d, J=0.8 Hz, 1H), 7.53 (d, J=0.8 Hz, 1H), 6.82-6.62 (m, 1H), 5.86 (t, J=55.4 Hz, 1H), 4.62 (t, J=6.4 Hz, 2H), 3.97-3.54 (m, 6H), 2.77-2.32 (m, 9H), 2.12 (dd, J=14.3, 2.7 Hz, 1H), 2.00-1.80 (m, 2H), 1.58 (dd, J=14.2, 11.6 Hz, 1H), 1.22 (d, J=6.3 Hz, 3H).

### Preparation S82

### tert-butyl (2′S, 7R)-2-bromo-2′-methyl-spiro[4,5-dihydrothieno[2,3-c]pyran-7,4′-piperidine]-1′-carboxylate (S82)

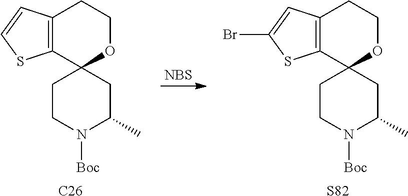

To a flask was added tert-butyl (2′S,7R)-2′-methylspiro[4,5-dihydrothieno[2,3-c]pyran-7,4′-piperidine]-1′-carboxylate C26 (860 mg, 2.659 mmol) in THF (15 mL) and NBS (465 mg, 2.613 mmol) under nitrogen. The reaction mixture was stirred overnight, before quenching with saturated bicarbonate solution and the organic solvent was evaporated. The aqueous layer was extracted with DCM. The combined organic layer was concentrated and purified by silica gel chromatography (Gradient: 0-20% EtOAc in heptane) to yield tert-butyl (2′S,7R)-2-bromo-2′-methyl-spiro[4,5-dihydrothieno[2,3-c]pyran-7,4′-piperidine]-1′-carboxylate (905 mg, 81%). LCMS m/z 402.08 [M+H]⁺.

### Compound 1032

### (2′S, 7R)-2′-methyl-1′-[[1-(2-methylsulfonylethyl)pyrazol-4-yl]methyl]-2-(2,2,2-trifluoroethyl)spiro[4,5-dihydrothieno[2,3-c]pyran-7,4′-piperidine](1032)

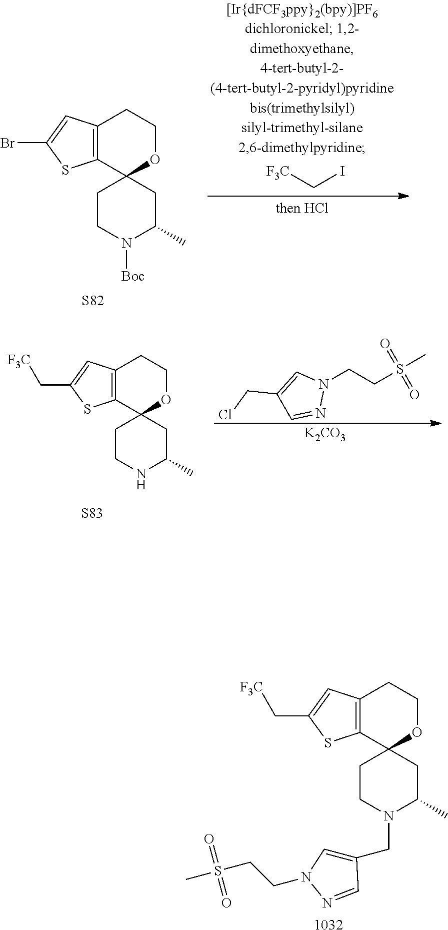

### Step 1. Synthesis of (2′S, 7R)-2′-methyl-2-(2,2,2-trifluoroethyl)spiro[4,5-dihydrothieno[2,3-c]pyran-7,4′-piperidine](S83)

Dichloronickel; 1,2-dimethoxyethane (3.2 mg, 0.014 mmol), 4-tert-butyl-2-(4-tert-butyl-2-pyridyl)pyridine (3.9 mg, 0.014 mmol) and [Ir{dFCFppy}₂(bpy)]PF₆ (1.4 mg, 0.0014 mmol), were weighed into a 1 dram vial. The reaction was capped with a septum and pump/purge (×3). To it was added tert-butyl (2′S,7R)-2-bromo-2′-methyl-spiro[4,5-dihydrothieno[2,3-c]pyran-7,4′-piperidine]-1′-carboxylate S82 (60 mg, 0.14 mmol) in DME (1 mL) and stirred for 10 min. Then 2,6-dimethylpyridine (31 mg, 0.29 mmol), bis(trimethylsilyl)silyl-trimethyl-silane (36 mg, 0.14 mmol) and 1,1,1-trifluoro-2-iodo-ethane (60 mg, 0.29 mmol) were added. The reaction mixture was irradiated in the Merck Photoreactor (100% LED Power/4700 RPM fan/1700 RPM stirring) for 2 h. The reaction was then diluted with DCM and washed with 1N HCl. The organic layer was concentrated in vacuo and added HCl (dioxane solution). After 2 h, the reaction mixture was concentrated. The crude material was purified by reversed-phase HPLC. Method: C18 Waters Sunfire column (30×150 mm, 5 micron). Gradient: MeCN in H₂O with 0.1% trifluoroacetic acid. The final product was isolated as (2′S,7R)-2′-methyl-2-(2,2,2-trifluoroethyl)spiro[4,5-dihydrothieno[2,3-c]pyran-7,4′-piperidine](2.4 mg, 5.5%). LCMS m/z 306.09 [M+H]⁺. ¹H NMR (400 MHz, DMSO-d₆) δ 8.73 (s, 1H), 8.32 (s, 1H), 6.85 (s, 1H), 4.09-3.82 (m, 5H), 3.28-3.03 (m, 2H), 2.61 (m, 2H), 2.16 (m, 2H), 1.89 (m, 1H), 1.74 (m, 1H), 1.21 (d, J=6.5 Hz, 3H).

### Step 2. Synthesis of (2′S, 7R)-2′-methyl-1′-[[1-(2-methylsulfonylethyl)pyrazol-4-yl]methyl]-2-(2,2,2-trifluoroethyl)spiro[4,5-dihydrothieno[2,3-c]pyran-7,4′-piperidine](1032)

Dissolved (2′S,7R)-2′-methyl-2-(2,2,2-trifluoroethyl)spiro[4,5-dihydrothieno[2,3-c]pyran-7,4′-piperidine]S83 (20 mg, 0.048 mmol) in MeCN (2 mL) under nitrogen. Then 4-(chloromethyl)-1-(2-methylsulfonylethyl)pyrazole (12.4 mg, 0.048 mmol) and K₂CO₃ (19.9 mg, 0.144 mmol) were added. The reaction mixture was heated to 70° C. overnight. Then it was quenched with water and extract with DCM (×3). The combined organic layer was concentrated and purified by silica gel chromatography (Gradient: 0-20% MeOH in DCM) to yield (2′S,7R)-2′-methyl-1′-[[1-(2-methylsulfonylethyl)pyrazol-4-yl]methyl]-2-(2,2,2-trifluoroethyl)spiro[4,5-dihydrothieno[2,3-c]pyran-7,4′-piperidine](4.2 mg, 18%). LCMS m/z 492.12 [M+H]⁺. ¹H NMR (400 MHz, DMSO-d₆) δ 7.72 (s, 1H), 7.40 (s, 1H), 6.77 (s, 1H), 4.51 (t, J=6.8 Hz, 2H), 3.90-3.62 (m, 7H), 3.46 (d, J=14.3 Hz, 1H), 2.77 (s, 3H), 2.45-2.30 (m, 5H), 1.91 (d, J=13.2 Hz, 2H), 1.66 (dt, J=13.9, 6.9 Hz, 1H), 1.44 (t, J=12.5 Hz, 1H), 1.10 (d, J=6.1 Hz, 3H).

### Compound 1033

### (2′S,7R)-2-cyclobutyl-2′-methyl-1′-[[1-(2-methylsulfonylethyl)pyrazol-4-yl]methyl]spiro[4,5-dihydrothieno[2,3-c]pyran-7,4′-piperidine](1033)

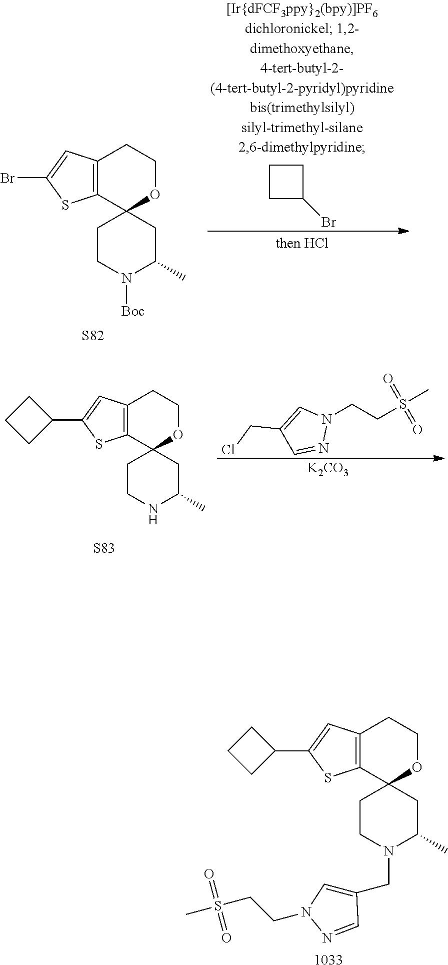

(2′S,7R)-2-cyclobutyl-2′-methyl-1′-[[1-(2-methylsulfonylethyl)pyrazol-4-yl]methyl]spiro[4,5-dihydrothieno[2,3-c]pyran-7,4′-piperidine]was prepared according the procedures as in the preparation of compound 1032. LCMS m/z 464.21 [M+H]⁺. ¹H NMR (400 MHz, DMSO-d₆) δ 7.72 (s, 1H), 7.40 (s, 1H), 6.52 (s, 1H), 4.51 (t, J=6.8 Hz, 2H), 3.84-3.43 (m, 7H), 2.77 (s, 3H), 2.56-2.26 (m, 7H), 2.11-1.59 (m, 7H), 1.50-1.39 (m, 1H), 1.09 (d, J=6.2 Hz, 3H).

### Compound 1034

### (2′S, 7R)-2-cyclopropyl-2′-methyl-1′-[[1-(2-methylsulfonylethyl)pyrazol-4-yl]methyl]spiro[4,5-dihydrothieno[2,3-c]pyran-7,4′-piperidine] (1034)

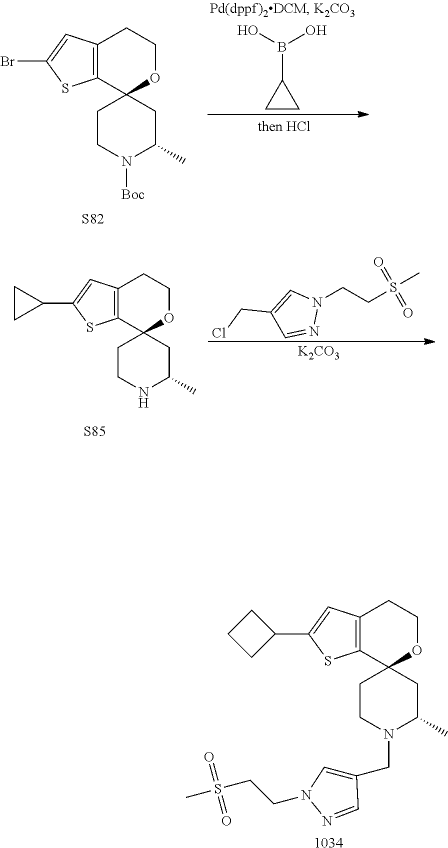

### Step 1. Synthesis of (2′S, 7R)-2-cyclopropyl-2′-methyl-spiro[4,5-dihydrothieno[2,3-c]pyran-7,4′-piperidine] (S85)

Added tert-butyl (2′S,7R)-2-bromo-2′-methyl-spiro[4,5-dihydrothieno[2,3-c]pyran-7,4′-piperidine]-1′-carboxylate S82 (120 mg, 0.2871 mmol), cyclopropylboronic acid (30 mg, 0.3493 mmol), Pd(dppf)Cl₂ (23.5 mg, 0.02871 mmol) and K₂CO₃ (80 mg, 0.5788 mmol) to DME (2 mL) under nitrogen. The reaction mixture was sparged with nitrogen for 5 min before heating to reflux overnight. The reaction mixture was then diluted with EtOAc and extracted from water (×2). The combined organic layer was concentrated in vacuo and then treated with HCl (1000 μL of 4 M in dioxane, 4.000 mmol) and stirred overnight. The reaction mixture was concentrated in vacuo. The crude material was purified by reversed-phase HPLC. Method: C18 Waters Sunfire column (30×150 mm, 5 micron). Gradient: MeCN in H₂O with 5 mM HCl. The product was isolated as (2′S,7R)-2-cyclopropyl-2′-methyl-spiro[4,5-dihydrothieno[2,3-c]pyran-7,4′-piperidine] (Hydrochloride salt) (14.6 mg, 16%). LCMS m/z 264.1 [M+H]⁺. ¹H NMR (400 MHz, DMSO-d₆) δ 8.68 (s, 1H), 8.28 (s, 1H), 6.54 (s, 1H), 3.85 (m, 2H), 3.42-3.04 (m, 4H), 2.17-2.00 (m, 3H), 1.90-1.64 (m, 2H), 1.20 (d, J=6.5 Hz, 4H), 1.01-0.92 (m, 2H), 0.65-0.58 (m, 2H).

### Step 2. Synthesis of (2′S, 7R)-2-cyclopropyl-2′-methyl-1′-[[1-(2-methylsulfonylethyl)pyrazol-4-yl]methyl]spiro[4,5-dihydrothieno[2,3-c]pyran-7,4′-piperidine] (1034)

To a solution of (2′S,7R)-2-cyclopropyl-2′-methyl-spiro[4,5-dihydrothieno[2,3-c]pyran-7,4′-piperidine](Hydrochloride salt) (11.5 mg, 0.038 mmol) in ACN (1 mL) was added K₂CO₃ (40 mg, 0.2894 mmol) and 4-(chloromethyl)-1-(2-methylsulfonylethyl)pyrazole (10 mg, 0.04491 mmol). The reaction mixture was stirred at 70° C. overnight before diluting with water and extracting with DCM. The combined organic layer was concentrated and purified by silica gel chromatography (Gradient: 0-20% MeOH in DCM) to yield (2′S,7R)-2-cyclopropyl-2′-methyl-1′-[[1-(2-methylsulfonylethyl)pyrazol-4-yl]methyl]spiro[4,5-dihydrothieno[2,3-c]pyran-7,4′-piperidine] (9.2 mg, 7%). LCMS m/z 450.48 [M+H]⁺. ¹H NMR (400 MHz, DMSO-d₆) δ 7.71 (s, 1H), 7.39 (s, 1H), 6.46 (s, 1H), 4.51 (t, J=6.8 Hz, 2H), 3.79-3.64 (m, 5H), 3.50-3.25 (m, 2H), 2.77 (s, 3H), 2.49-2.26 (m, 2H), 2.05-1.78 (m, 3H), 1.70-1.36 (m, 4H), 1.08 (d, J=6.2 Hz, 3H), 0.92 (dd, J=8.2, 2.3 Hz, 2H), 0.60 (dd, J=4.9, 2.1 Hz, 2H).

### Compound 1035

### (2′S,7R)-2′-methyl-1′-[[1-(2-methylsulfonylethyl)pyrazol-4-yl]methyl]spiro[4,5-dihydrothieno[2,3-c]pyran-7,4′-piperidine]-2-carbonitrile (1035)

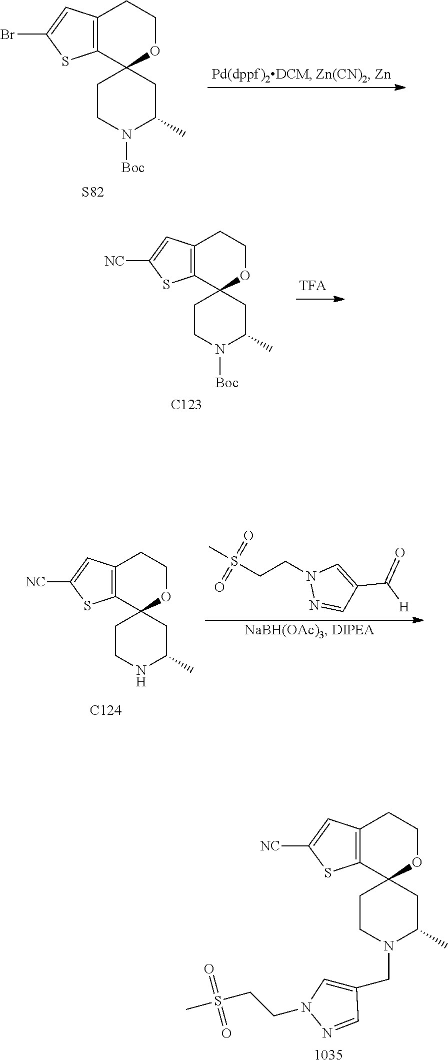

### Step 1. Synthesis of tert-butyl (2′S, 7R)-2-cyano-2′-methyl-spiro[4,5-dihydrothieno[2,3-c]pyran-7,4′-piperidine]-1′-carboxylate (C123)

To a 2-dram vial was charged with tert-butyl (2′S,7R)-2-bromo-2′-methyl-spiro[4,5-dihydrothieno[2,3-c]pyran-7,4′-piperidine]-1′-carboxylate S82 (100 mg, 0.2485 mmol), Pd(dppf)Cl₂ (21 mg, 0.02572 mmol), dicyanozinc (24 μL, 0.3781 mmol), Zn (5 mg, 0.07644 mmol). DMA (2.4 mL) was added and the reaction mixture was purged with N₂ for 5 min. The reaction was heated at 90° C. for 20 h. The reaction mixture was diluted with EtOAc and quenched with NH₄OH (2.5M). The organic layer was separated, and the aqueous layer was extracted with EtOAc (×3), and the combined organic extracts were concentrated in vacuo. The crude was purified by silica gel chromatography (Gradient: 0-50% EtOAc in hexanes) to yield tert-butyl (2′S,7R)-2-cyano-2′-methyl-spiro[4,5-dihydrothieno[2,3-c]pyran-7,4′-piperidine]-1′-carboxylate (110 mg, 127%). LCMS m/z 348.95 [M+H]⁺. The product was carried forward without further purification assuming quantitative yield.

### Step 2. Synthesis of (2′S, 7R)-2′-methylspiro[4,5-dihydrothieno[2,3-c]pyran-7,4′-piperidine]-2-carbonitrile (C124)

To a 2-dram vial charged with tert-butyl (2′S,7R)-2-cyano-2′-methyl-spiro[4,5-dihydrothieno[2,3-c]pyran-7,4′-piperidine]-1′-carboxylate C123 (86 mg, 0.2468 mmol) and DCM (0.8 mL) was added TFA (200 μL, 2.596 mmol). The resulting yellow solution was stirred at rt. The volatile was removed in vacuo to afford the crude product: (2′S,7R)-2′-methylspiro[4,5-dihydrothieno[2,3-c]pyran-7,4′-piperidine]-2-carbonitrile (Trifluoroacetate salt) (89 mg, 95%). LCMS m/z 249.0 [M+H]⁺. ¹H NMR (300 MHz, Methanol-d₄) δ 7.54 (s, 1H), 4.00 (td, J=5.7, 2.1 Hz, 2H), 3.62 (s, 1H), 3.45-3.30 (m, 2H), 2.77 (t, J=5.5 Hz, 2H), 2.41-2.18 (m, 2H), 1.99 (ddd, J=14.9, 12.0, 5.9 Hz, 1H), 1.82 (dd, J=14.7, 12.2 Hz, 1H), 1.34 (d, J=6.6 Hz, 3H). The product was carried forward without further purification.

### Step 3. Synthesis of (2′S, 7R)-2′-methyl-1′-[[1-(2-methylsulfonylethyl)pyrazol-4-yl]methyl]spiro[4,5-dihydrothieno[2,3-c]pyran-7,4′-piperidine]-2-carbonitrile (1035)

To a 2-dram vial was charged (2′S,7R)-2′-methylspiro[4,5-dihydrothieno[2,3-c]pyran-7,4′-piperidine]-2-carbonitrile (Trifluoroacetate salt) C124 (35 mg, 0.09659 mmol) and DCM (1 mL). 1-(2-methylsulfonylethyl)pyrazole-4-carbaldehyde (39 mg, 0.1928 mmol) was added followed by sodium triacetoxyborohydride (61 mg, 0.2892 mmol). The resulting mixture was stirred at rt. N-ethyl-N-isopropyl-propan-2-amine (34 μL, 0.1952 mmol) and 1-(2-methylsulfonylethyl)pyrazole-4-carbaldehyde (20 mg, 0.10 mmol) were added and the reaction was stirred at rt overnight. The reaction was quenched slowly with saturated NaHCO₃ solution, extracted with DCM, and the combined organic extracts were concentrated in vacuo. The crude material was purified by reversed-phase HPLC. Method: C18 Waters Sunfire column (30×150 mm, 5 micron). Gradient: MeCN in H₂O with 0.1% trifluoroacetic acid. The product was isolated as (2′S,7R)-2′-methyl-1′-[[1-(2-methylsulfonylethyl)pyrazol-4-yl]methyl]spiro[4,5-dihydrothieno[2,3-c]pyran-7,4′-piperidine]-2-carbonitrile (Trifluoroacetate salt) (35.4 mg, 63%). LCMS m/z 435.0 [M+H]⁺. ¹H NMR (300 MHz, Methanol-d₄) δ 8.00 (s, 1H), 7.73 (s, 1H), 7.54 (s, 1H), 4.76-4.66 (m, 2H), 4.56 (d, J=14.1 Hz, 1H), 4.30 (d, J=14.1 Hz, 1H), 3.94 (q, J=5.1 Hz, 2H), 3.73 (t, J=6.2 Hz, 2H), 3.55 (s, 1H), 3.44-3.33 (m, 3H), 2.88 (s, 3H), 2.74 (t, J=5.5 Hz, 2H), 2.49-2.26 (m, 1H), 2.11-1.89 (m, 2H), 1.54 (d, J=6.4 Hz, 3H).

### Compound 1036

### (2′S, 7R)-2-ethyl-2′-methyl-1′-[[1-(2-methylsulfonylethyl)triazol-4-yl]methyl]spiro[4,5-dihydrothieno[2,3-c]pyran-7,4′-piperidine](1036)

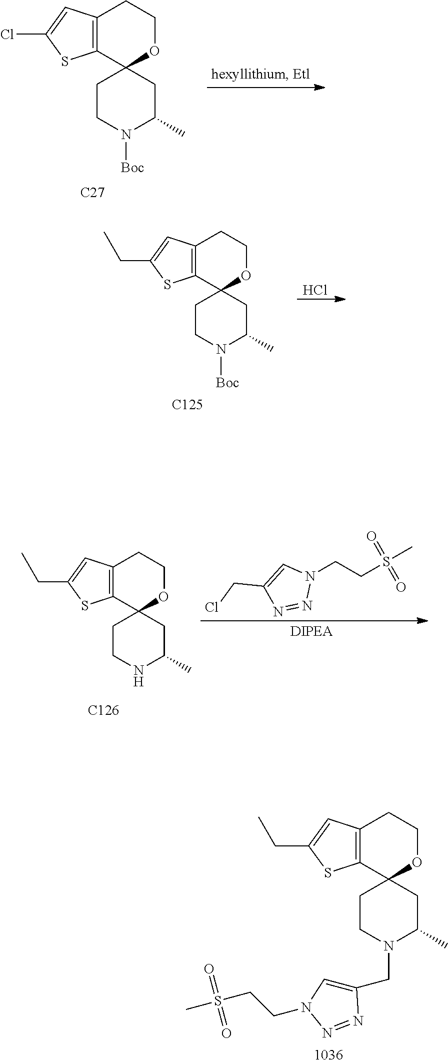

### Step 1. Synthesis of tert-butyl (2′S, 7R)-2-ethyl-2′-methyl-spiro[4,5-dihydrothieno[2,3-c]pyran-7,4′-piperidine]-1′-carboxylate (C125)

Tert-butyl (2′S,7R)-2-chloro-2′-methyl-spiro[4,5-dihydrothieno[2,3-c]pyran-7,4′-piperidine]-1′-carboxylate C27 (110 mg, 0.3074 mmol) was dissolved in THF (4 mL) and cooled to −78° C. hexyllithium (400 μL of 2.3 M, 0.9200 mmol) was added dropwise and the solution was allowed to stir for 60 min. Iodoethane (25 μL, 0.3126 mmol) was added to the solution and it was warmed to rt. After 12 h, the reaction was quenched with ammonium chloride aqueous solution and extracted with EtOAc, washed with brine and dried with sodium sulfate. The combined organic layer was concentrated in vacuo to provide tert-butyl (2′S,7R)-2-ethyl-2′-methyl-spiro[4,5-dihydrothieno[2,3-c]pyran-7,4′-piperidine]-1′-carboxylate (120 mg, 25%). LCMS m/z 352.52 [M+H]⁺.

### Step 2. Synthesis of (2′S, 7R)-2-ethyl-2′-methyl-spiro[4,5-dihydrothieno[2,3-c]pyran-7,4′-piperidine] (C126)

Tert-butyl (2′S,7R)-2-ethyl-2′-methyl-spiro[4,5-dihydrothieno[2,3-c]pyran-7,4′-piperidine]-1′-carboxylate C125 (120 mg, 0.3414 mmol) was dissolved in dioxane (4.3 mL) and HCl (850 μL of 4 M, 3.400 mmol) was added. The solution was stirred overnight. After 12 h, the solution was concentrated in vacuo to provide (2′S,7R)-2-ethyl-2′-methyl-spiro[4,5-dihydrothieno[2,3-c]pyran-7,4′-piperidine] (85.82 mg, 100%), which was carried forward to the next step without purification. LCMS m/z 252.46 [M+H]⁺.

### Step 3. Synthesis of (2′S,7R)-2-ethyl-2′-methyl-1′-[[1-(2-methylsulfonylethyl)triazol-4-yl]methyl]spiro[4,5-dihydrothieno[2,3-c]pyran-7,4′-piperidine](1046)

(2′S,7R)-2-ethyl-2′-methyl-spiro[4,5-dihydrothieno[2,3-c]pyran-7,4′-piperidine] C126 (85.82 mg, 0.34 mmol) was dissolved in DCE (4.3 mL) and 4-(chloromethyl)-1-(2-methylsulfonylethyl)triazole (76 mg, 0.3398 mmol) and DIPEA (60 μL, 0.3445 mmol) were added. The resulting solution was stirred at rt and heated to 65° C. After 24 h, the solution was cooled and partitioned between DCM and 1N NaOH. The organic layer was collected through a phase separator tube then concentrated in vacuo. Purification by reversed-phase HPLC. Method: C18 Waters Sunfire column (30×150 mm, 5 micron). Gradient: MeCN in H₂O with 0.1% trifluoroacetic acid. The product was isolated as (2′S,7R)-2-ethyl-2′-methyl-1′-[[1-(2-methylsulfonylethyl)triazol-4-yl]methyl]spiro[4,5-dihydrothieno[2,3-c]pyran-7,4′-piperidine](Trifluoroacetate salt) (41 mg, 43%). LCMS m/z 439.2 [M+H]⁺. ¹H NMR (300 MHz, Methanol-d₄) δ 8.33 (s, 1H), 6.63-6.40 (m, 1H), 5.07-4.91 (m, 2H), 4.74-4.48 (m, 2H), 4.00-3.74 (m, 4H), 3.55 (d, J=11.3 Hz, 1H), 3.46-3.35 (m, 2H), 3.06-2.93 (m, 3H), 2.77 (dt, J=8.3, 7.0 Hz, 2H), 2.61 (dt, J=10.7, 7.1 Hz, 2H), 2.37-1.95 (m, 4H), 1.76-1.45 (m, 3H), 1.25 (t, J=7.5 Hz, 3H).

### Compound 1037

### (2′S, 7R)-2-ethyl-2′-methyl-1′-[[1-(2-methylsulfonylethyl)pyrazol-4-yl]methyl]spiro[4,5-dihydrothieno[2,3-c]pyran-7,4′-piperidine]-4-ol (1037)

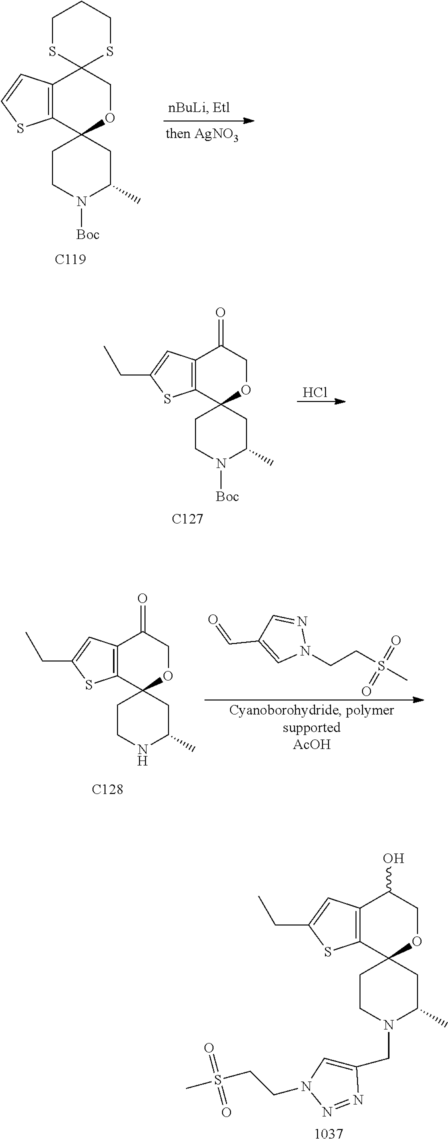

### Step 1. Synthesis of tert-butyl (2S,4R)-2′-ethyl-2-methyl-4′-oxo-spiro[piperidine-4,7′-thieno[2,3-c]pyran]-1-carboxylate (C127)

To a solution of tert-butyl (6″S,7′R)-6″-methyl-5′H-dispiro[1,3-dithiane-2,4′-thieno[2,3-c]pyran-7′,4″-piperidine]-1″-carboxylate C119 (600 mg, 1.403 mmol) in THF (16 mL) at −78° C. was added n-BuLi (582 μL of 2.41 M, 1.403 mmol) dropwise. The reaction was stirred for 1 h, at which point iodoethane (224 μL, 2.801 mmol) was added as a solution in THF at −78° C. After stirring for 1 h, the reaction mixture was warmed to rt. The reaction was quenched with NH₄Cl aqueous solution and partitioned between DCM and NH₄Cl aqueous solution. The aqueous layer was extracted with DCM (×3) and the combined organic layer was washed with brine, dried with sodium sulfate and the solvent was removed in vacuo to provide tert-butyl (6″S,7′R)-2′-ethyl-6″-methyl-5′H-dispiro[1,3-dithiane-2,4′-thieno[2,3-c]pyran-7′,4″-piperidine]-1″-carboxylate.

The crude material was dissolved in Ethanol (24 mL) and silver nitrate (137 μL, 3.510 mmol) was added and the reaction was heated to 50° C. After 2 h, the solution was partitioned between DCM and water, and the organic layer was collected through a phase separator. The solvent was removed in vacuo. Purification by silica gel chromatography (Gradient: 0-60% EtOAc in heptane) yielded the product tert-butyl (2S,4R)-2′-ethyl-2-methyl-4′-oxo-spiro[piperidine-4,7′-thieno[2,3-c]pyran]-1-carboxylate (150 mg, 29%). LCMS m/z 366.33 [M+H]⁺. ¹H NMR (300 MHz, Methanol-d₄) δ 7.01 (t, J=1.1 Hz, 1H), 4.29 (s, 2H), 4.06 (dq, J=12.4, 6.2 Hz, 1H), 3.81 (ddd, J=14.4, 6.8, 3.3 Hz, 1H), 3.40-3.35 (m, 1H), 2.84 (qd, J=7.5, 1.2 Hz, 2H), 2.48-2.21 (m, 2H), 2.03-1.71 (m, 2H), 1.48 (s, 11H), 1.37-1.13 (m, 7H).

### Step 2. Synthesis of (2S,4R)-2′-ethyl-2-methyl-spiro[piperidine-4,7′-thieno[2,3-c]pyran]-4′-one (C128)

Tert-butyl (2S,4R)-2′-ethyl-2-methyl-4′-oxo-spiro[piperidine-4,7′-thieno[2,3-c]pyran]-1-carboxylate C127 (90 mg, 0.2462 mmol) was dissolved in dioxane (500 μL) and HCl (620 μL of 4 M in dioxane, 2.480 mmol) was added. The reaction mixture was stirred at rt for 30 min. The solvent was removed in vacuo. Purification by reversed-phase HPLC. Method: C18 Waters Sunfire column (30×150 mm, 5 micron). Gradient: MeCN in H₂O with 0.1% trifluoroacetic acid. The product was isolated as (2S,4R)-2′-ethyl-2-methyl-spiro[piperidine-4,7′-thieno[2,3-c]pyran]-4′-one (Trifluoroacetate salt) (72 mg, 77%). LCMS m/z 266.28 [M+H]⁺. ¹H NMR (300 MHz, Methanol-d₄) δ 7.08 (t, J=1.1 Hz, 1H), 4.39 (d, J=0.7 Hz, 2H), 3.61 (ddp, J=9.3, 6.4, 2.7 Hz, 1H), 3.45-3.34 (m, 2H), 2.86 (qd, J=7.5, 1.1 Hz, 2H), 2.59-2.36 (m, 2H), 2.19-1.85 (m, 2H), 1.44-1.17 (m, 6H).

### Step 3. Synthesis of (2′S, 7R)-2-ethyl-2′-methyl-1′-[[1-(2-methylsulfonylethyl)pyrazol-4-yl]methyl]spiro[4,5-dihydrothieno[2,3-c]pyran-7,4′-piperidine]-4-ol (1037)

(2S,4R)-2′-ethyl-2-methyl-spiro[piperidine-4,7′-thieno[2,3-c]pyran]-4′-one (Trifluoroacetate salt) C128 (31 mg, 0.08171 mmol) and 1-(2-methylsulfonylethyl)pyrazole-4-carbaldehyde (25 mg, 0.1236 mmol) were dissolved in DCM (2 mL) and acetic acid (23 mg, 0.3830 mmol) was added to the solution. The solution was transferred to a microwave tube (5 mL) and cyanoborohydride, polymer-supported (122 mg of 2 mmol/g, 0.2440 mmol) was added. The solution was capped and heated to 95° C. in a microwave reactor for 2 h. The solution was filtered, and the solvent removed in vacuo. Purification by reversed-phase HPLC. Method: C18 Waters Sunfire column (30×150 mm, 5 micron). Gradient: MeCN in H₂O with 0.1% trifluoroacetic acid yielded (2′S,7R)-2-ethyl-2′-methyl-1′-[[1-(2-methylsulfonylethyl)pyrazol-4-yl]methyl]spiro[4,5-dihydrothieno[2,3-c]pyran-7,4′-piperidine]-4-ol (Trifluoroacetate salt) (16 mg, 31%). LCMS m/z 454.34 [M+H]⁺. ¹H NMR (300 MHz, Methanol-d₄) δ 7.99 (s, 1H), 7.72 (d, J=0.8 Hz, 1H), 6.73 (q, J=1.0 Hz, 1H), 4.78-4.64 (m, 2H), 4.64-4.38 (m, 2H), 4.28 (d, J=14.1 Hz, 1H), 4.02-3.63 (m, 4H), 3.36 (dd, J=5.0, 2.3 Hz, 1H), 3.28-3.16 (m, 1H), 2.92-2.63 (m, 6H), 2.46-2.23 (m, 2H), 2.16-1.73 (m, 2H), 1.53 (dd, J=6.5, 4.1 Hz, 3H), 1.26 (t, J=7.5 Hz, 3H).

### Compound 1038

### (2′S,7R)-2-ethyl-2′-methyl-1′-[[1-(2-methylsulfonylethyl)triazol-4-yl]methyl]spiro[4,5-dihydrothieno[2,3-c]pyran-7,4′-piperidine]-4-ol (1038)

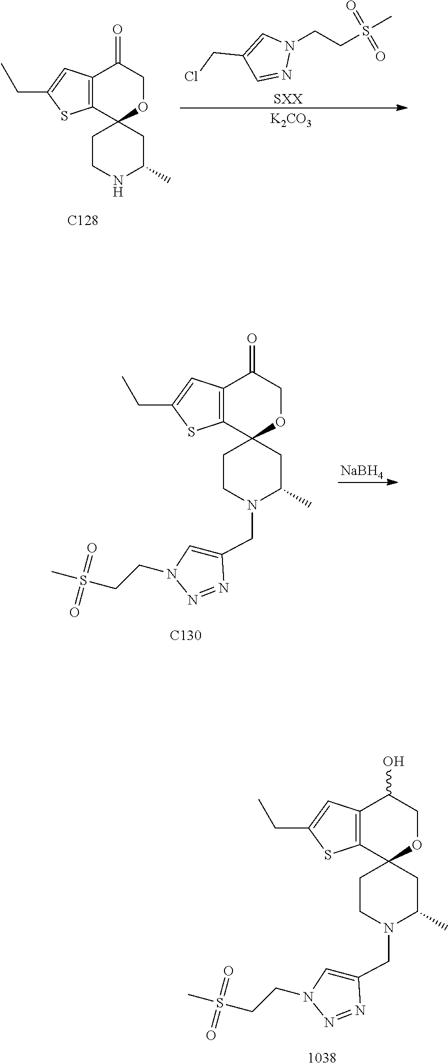

### Step 1. Synthesis of (2S,4R)-2′-ethyl-2-methyl-1-[[1-(2-methylsulfonylethyl)triazol-4-yl]methyl]spiro[piperidine-4,7′-thieno[2,3-c]pyran]-4′-one (C130)

To a solution of (2S,4R)-2′-ethyl-2-methyl-spiro[piperidine-4,7′-thieno[2,3-c]pyran]-4′-one (Trifluoroacetate salt) C128 (10 mg, 0.02636 mmol) in acetonitrile (429.9 μL) was added 4-(chloromethyl)-1-(2-methylsulfonylethyl)triazole (Hydrochloride salt) (10 mg, 0.03844 mmol) and potassium carbonate (11.4 mg, 0.08249 mmol). The mixture was stirred at 70° C. for 3 h. The mixture was cooled to rt, diluted with water and DCM, and the organic later was collected through a phase separator. The organic layer was collected and the solvent was removed in vacuo to give crude product (2S,4R)-2′-ethyl-2-methyl-1-[[1-(2-methylsulfonylethyl)triazol-4-yl]methyl]spiro[piperidine-4,7′-thieno[2,3-c]pyran]-4′-one (11.9 mg, 100%). LCMS m/z 453.22 [M+H]⁺. The material was carried forward to the next step without purification.

### Step 2. Synthesis of (2′S,7R)-2-ethyl-2′-methyl-1′-[[1-(2-methylsulfonylethyl)triazol-4-yl]methyl]spiro[4,5-dihydrothieno[2,3-c]pyran-7,4′-piperidine]-4-ol (1038)

Dissolved (2S,4R)-2′-ethyl-2-methyl-1-[[1-(2-methylsulfonylethyl)triazol-4-yl]methyl]spiro[piperidine-4,7′-thieno[2,3-c]pyran]-4′-one C130 (15.8 mg, 0.03491 mmol) in DCM (630 μL) and Methanol (158 μL) under N₂, followed by addition of NaBH₄ (7.8 mg, 0.2073 mmol). After stirring at rt for 1 h, the reaction mixture was washed with water, and with DCM and the organic layer was collected through a phase separator. Purification by reversed-phase HPLC. Method: C18 Waters Sunfire column (30×150 mm, 5 micron). Gradient: MeCN in H₂O with 0.1% trifluoroacetic acid afforded (2′S,7R)-2-ethyl-2′-methyl-1′-[[1-(2-methylsulfonylethyl)triazol-4-yl]methyl]spiro[4,5-dihydrothieno[2,3-c]pyran-7,4′-piperidine]-4-ol (Trifluoroacetate salt) (2.3 mg, 12%). LCMS m/z 455.33 [M+H]⁺. ¹H NMR (300 MHz, Methanol-d₄) δ 8.06 (s, 1H), 6.67 (d, J=1.3 Hz, 1H), 4.92 (t, J=6.6 Hz, 3H), 4.40 (dd, J=6.6, 3.5 Hz, 1H), 4.11-3.61 (m, 6H), 2.93-2.54 (m, 9H), 2.14-1.50 (m, 3H), 1.25 (q, J=4.2, 3.6 Hz, 6H).

### Compound 1040

### 2-chloro-2′-cyclopropyl-1′-[[1-(2-methylsulfonylethyl)pyrazol-4-yl]methyl]spiro[4,5-dihydrothieno[2,3-c]pyran-7,4′-piperidine](1040)

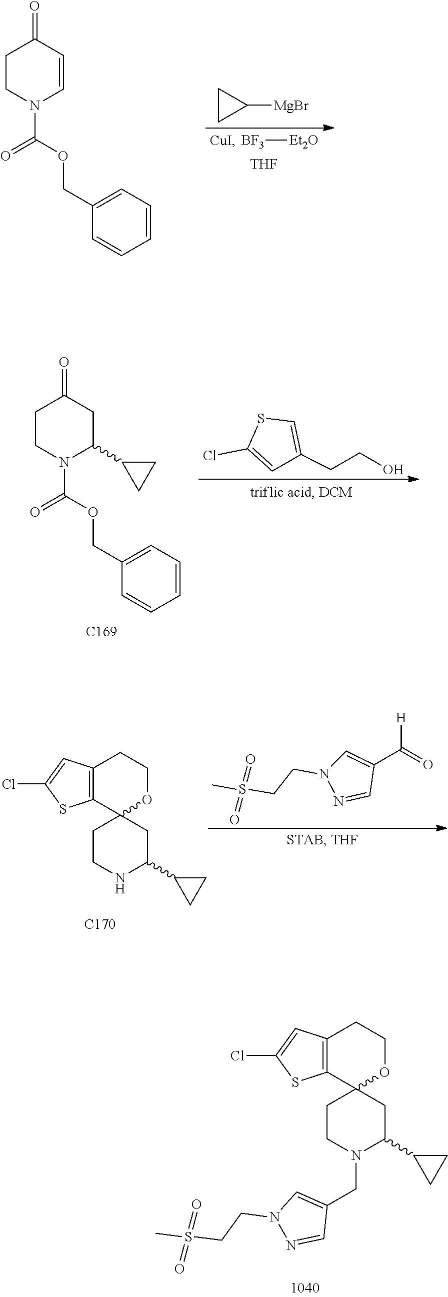

### Step 1: Synthesis of benzyl 2-cyclopropyl-4-oxo-piperidine-1-carboxylate (C169)

To a round bottom flask under nitrogen atmosphere was added CuI (1.62 g, 8.506 mmol) in THF (11 mL) at −78° C. Bromo(cyclopropyl)magnesium (9 mL of 1 M, 9.000 mmol) was added, and the mixture was stirred at −78° C. for 30 minutes, then diethyloxonio(trifluoro)boranuide (80 μL, 0.6482 mmol) was added. The mixture was stirred at −78° C. for another 10 minutes, and benzyl 4-oxo-2,3-dihydropyridine-1-carboxylate (1 g, 4.324 mmol) in 4 mL THF was added. Stirring was continued at −78° C. for 5 hours. The reaction was quenched with saturated NH₄Cl. Organic layer was extracted with CH₂C₁₂ (3×) with a phase separator, and concentrated. The crude product was purified via normal phase flash column chromatography (Gradient: 0-50% EtOAc/Heptanes) to provide benzyl 2-cyclopropyl-4-oxo-piperidine-1-carboxylate (603 mg, 37%). ¹H NMR (300 MHz, Chloroform-d) δ 7.23 (d, J=11.5 Hz, 5H), 5.12-4.96 (m, 2H), 4.30 (dd, J=14.1, 7.0 Hz, 1H), 3.83 (t, J=8.2 Hz, 1H), 3.43-3.27 (m, 1H), 2.53 (dd, J=14.4, 6.8 Hz, 1H), 2.46-2.16 (m, 3H), 0.80 (dddd, J=10.1, 8.0, 5.0, 3.1 Hz, 1H), 0.56-0.11 (m, 4H). ESI-MS m/z 274.07 [M+H]⁺.

### Step 2: Synthesis of 2-chloro-2′-cyclopropyl-spiro[4,5-dihydrothieno[2,3-c]pyran-7,4′-piperidine](C170)

Benzyl 2-cyclopropyl-4-oxo-piperidine-1-carboxylate (580 mg, 2.122 mmol) was dissolved in CH₂Cl₂ (12 mL) at 0° C. and treated with triflic acid (750 μL, 8.476 mmol). The solution was stirred for 10 minutes, then 2-(5-chloro-3-thienyl)ethanol (300 μL) was added. The reaction was allowed to warm to ambient temperature and stirred for 20 minutes. The reaction was quenched with NaHCO₃ and extracted with CH₂C₁₂ (3×) with a phase separator. The crude product was purified by normal phase flash column chromatography (Gradient: 0-80% EtOAc/Hep) to provide 2-chloro-2′-cyclopropyl-spiro[4,5-dihydrothieno[2,3-c]pyran-7,4′-piperidine] (332.8 mg, 50%) as a mixture of the corresponding trans enantiomers. ¹H NMR (300 MHz, Chloroform-d) δ 6.60 (s, 1H), 3.95-3.74 (m, 2H), 3.35 (ddd, J=23.5, 12.0, 4.8 Hz, 2H), 2.71-2.52 (m, 3H), 2.31 (d, J=14.6 Hz, 1H), 2.18-1.95 (m, 3H), 1.09 (dq, J=12.7, 7.7, 5.7 Hz, 1H), 0.77-0.52 (m, 3H), 0.29 (d, J=7.0 Hz, 1H). ESI-MS m/z 284.06 [M+H]⁺.

### Step 3: Synthesis of 2-chloro-2′-cyclopropyl-1′-[[1-(2-methylsulfonylethyl)pyrazol-4-yl]methyl]spiro[4,5-dihydrothieno[2,3-c]pyran-7,4′-piperidine](1040)

2-chloro-2′-cyclopropyl-spiro[4,5-dihydrothieno[2,3-c]pyran-7,4′-piperidine](59 mg, 0.2039 mmol), 1-(2-methylsulfonylethyl)pyrazole-4-carbaldehyde (62 mg, 0.3066 mmol), and triacetoxyboranuide (Sodium salt) (144 mg, 0.6794 mmol) were added to a microwave vial and dissolved in THF (1.4 mL). The reaction was stirred at 55° C. for 2 hours. The reaction mixture was quenched with NaHCO₃ and extracted with CH₂C₁₂ (3×) with a phase separator. The crude product was purified via normal phase chromatography (Gradient: 0-6% MeOH/DCM). To provide the desired 2-chloro-2′-cyclopropyl-1′-[[1-(2-methylsulfonylethyl)pyrazol-4-yl]methyl]spiro[4,5-dihydrothieno[2,3-c]pyran-7,4′-piperidine](Trifluoroacetate salt) (42.2 mg, 31%). ¹H NMR (300 MHz, MeOD) δ 7.96 (s, 1H), 7.71 (s, 1H), 6.75 (s, 1H), 5.10 (d, J=13.8 Hz, 1H), 4.71 (t, J=6.2 Hz, 2H), 4.07 (d, J=13.8 Hz, 1H), 3.93 (q, J=5.4 Hz, 2H), 3.72 (t, J=6.2 Hz, 2H), 3.19 (t, J=12.3 Hz, 1H), 2.90 (s, 3H), 2.64 (q, J=4.7 Hz, 2H), 2.47 (d, J=14.9 Hz, 1H), 2.27 (d, J=15.9 Hz, 1H), 2.09-1.98 (m, 1H), 1.92-1.81 (m, 1H), 1.35-1.21 (m, 1H), 1.02 (d, J=8.3 Hz, 2H), 0.90-0.70 (m, 2H), 0.35 (dd, J=9.5, 4.8 Hz, 1H). ESI-MS m/z 470.41 [M+H]⁺.

### Compound 1041

### 2,2′-diethyl-1-((1-methyl-1H-pyrazol-4-yl)methyl)-6′,7′-dihydrospiro[piperidine-4,4′-thieno [3,2-c]pyran] (1041)

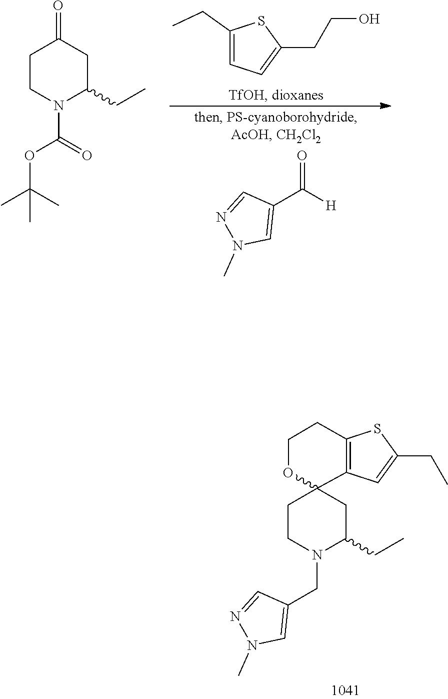

To a microwave vial with stir bar was added tert-butyl 2-ethyl-4-oxo-piperidine-1-carboxylate (90 mg, 0.4 mmol), 2-(5-ethyl-2-thienyl)ethanol (75 mg, 0.48 mmol) in dioxane (2.0 mL). Trifluoromethanesulfonic acid was added (0.1 mL, 1.13 mmol) and the reaction was stirred at ambient temperature for 2 hours. At this time the reaction was concentrated, and the crude intermediate redissolved in CH₂Cl₂ (2.0 mL). To the solution was added (polystyrylmethyl)trimethylammonium cyanoborohydride cross-linked with 10% DVB (30-50 mesh) (2.6-3.0 mmol/g) (500 mg, 1.0 mmol), acetic acid (0.13 mL, 2.0 mmol), and 1-methylpyrazole-4-carbaldehyde (88 mg, 0.8 mmol). The microwave vial was capped and irradiated under microwave conditions to 110° C. for 20 minutes. The mixture was then filtered, concentrated. Purification by reversed-phase HPLC. Method: C18 Waters Sunfire column (30×150 mm, 5 micron). Gradient: MeCN in H₂O with 0.1% trifluoroacetic acid, to provide 2,2′-diethyl-1-((1-methyl-1H-pyrazol-4-yl)methyl)-6′,7′-dihydrospiro[piperidine-4,4′-thieno[3,2-c]pyran] (13.7 mg, 7%). ESI-MS m/z 360.23 [M+H]⁺.

### Compound 1042

### 2″-ethyl-1′-((1-methyl-1H-pyrazol-4-yl)methyl)-6″,7″-dihydrodispiro[cyclobutane-1,2′-piperidine-4′,4″-thieno[3,2-c]pyran] (1042)

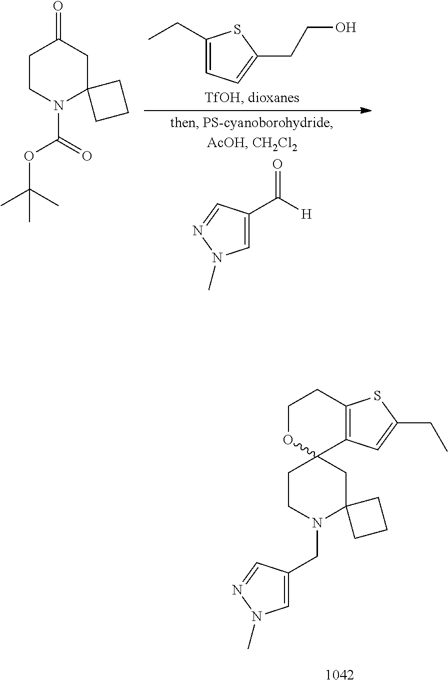

Compound 1042 was prepared following the method described for compound 1041, using the oxo-piperidine intermediate tert-butyl 8-oxo-5-azaspiro[3.5]nonane-5-carboxylate. This provided the product 2″-ethyl-1′-((1-methyl-1H-pyrazol-4-yl)methyl)-6″,7″-dihydrodispiro [cyclobutane-1,2′-piperidine-4′,4″-thieno[3,2-c]pyran] (12 mg, 6%). ESI-MS m/z 372.22 [M+H]⁺.

### PREPARATION OF COMMON INTERMEDIATES

### Preparation S86

### 1-(2-methylsulfonylethyl)triazole-4-carbaldehyde (S86)

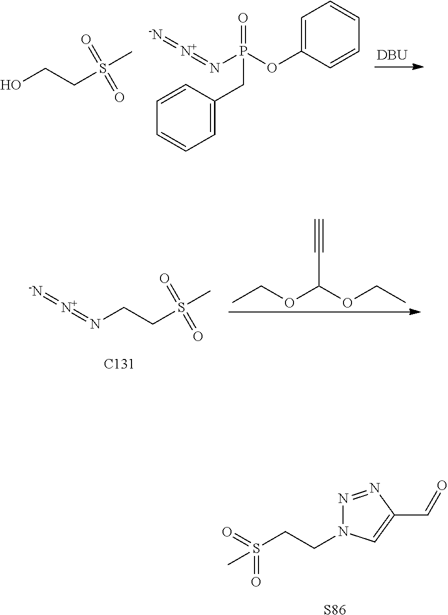

### Step 1: 1-azido-2-methylsulfonyl-ethane (C131)

A solution of 2-methylsulfonylethanol (5 g, 0.0403 mol) and Diphenyl phosphoryl azide (8.8614 g, 0.0322 mol) in Toluene (50.000 mL) was stirred at 0° C. for 10 min and DBU (5.5262 g, 5.4285 mL, 0.0363 mol) was added drop-wise at 0° C. over 10 minutes and the reaction was stirred at room temperature for 16 hours. Reaction mixture was quenched with water (25 mL) and ethyl acetate (100 mL) and stirred for 20 min. Organic layer was separated and aqueous layer was again extracted with ethyl acetate (100 mL×2). The organic layer was dried over sodium sulfate and concentrated. Purification by silica gel chromatography (Gradient: 0-100% ethyl acetate in pet ether) gave 1-azido-2-methylsulfonyl-ethane C131 (5.2 g, 86%) ¹H NMR (400 MHz, DMSO-d6) δ=3.77-3.73 (t, J=8.8 Hz, 2H), 3.44-3.42 (t, J=8.8 Hz, 2H), 3.03 (s, 3H).

### Step 2: 1-(2-methylsulfonylethyl)triazole-4-carbaldehyde (S86)

A mixture of 3,3-diethoxyprop-1-yne (555 μL, 3.897 mmol), 1-azido-2-methylsulfonyl-ethane C131 (600 mg, 4.022 mmol), CuSO4 (15 mg, 0.09398 mmol), 1-(1-benzyltriazol-4-yl)-N,N-bis[(1-benzyltriazol-4-yl)methyl]methanamine (100 mg, 0.1885 mmol), and sodium ascorbate (700 mg, 3.974 mmol) in MeOH (12 mL)/water (3 mL) was heated to 60° C. for 2 hours. The reaction was cooled to room temperature, concentrated, and diluted in EtOAc (100 mL) and water (50 mL). The layers were split and the aqueous layer was extracted with EtOAc (50 mL). The layers were combined and dried, diluted in 1 N HCl (20 mL), and stirred overnight. At this time, the solution was concentrated to yield 1-(2-methylsulfonylethyl)triazole-4-carbaldehyde (Hydrochloride salt) S86 (553 mg, 59%)¹H NMR (400 MHz, Methanol-d4) δ 8.07 (s, 1H), 5.58-5.45 (m, 1H), 4.89-4.82 (m, 2H), 3.76-3.67 (m, 2H), 3.24 (s, 3H). LCMS m/z 204.47 [M+H]⁺.

### Preparation S87

### 1-(2-methylsulfonylethyl)pyrazole-4-carbaldehyde (S87)

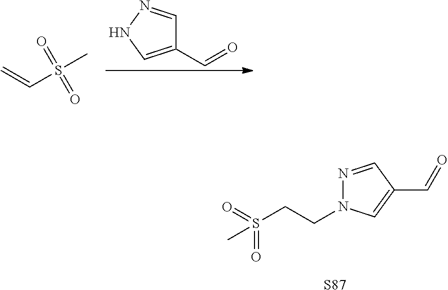

### Step 1: 1-(2-methylsulfonylethyl)pyrazole-4-carbaldehyde (S87)

A solution of 11H-pyrazole-4-carbaldehyde (10 g, 104.1 mmol) 11-methylsulfonylethylene (10 mL, 114.2 mmol) and Potassium Carbonate (25 g, 180.9 mmol) in tetrahydrofuran (200 mL) was stirred at 60° C. After stirring overnight, the mixture was cooled to room temperature and concentrated to dryness. The product was suspended in diethyl ether (100 mL) to triturate the product and stirred for 2 h. The product was filtered and dried overnight to yield 11-(2-methylsulfonylethyl)pyrazole-4-carbaldehyde S87 (20280 mg, 83%) 11H NMR (400 MHz, DMSO-d6) δ 9.80 (s, 1H), 8.54 (d, J=0.7 Hz, 1H), 8.05 (d, J=0.7 Hz, 1H), 4.64 (t, J=6.8 Hz, 2H), 3.80-3.67 (m, 2H), 2.96 (d, J=0.7 Hz, 3H). LCMS m/z 203.01 [M+H]⁺.

### Preparation S88

### 1-[2-[tert-butyl(dimethyl)silyl]oxyethyl]pyrazole-4-carbaldehyde (S88)

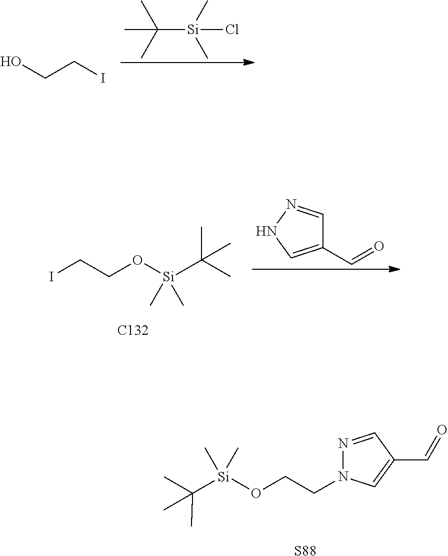

### Step 1: tert-butyl-(2-iodoethoxy)-dimethyl-silane (C132)

To a stirred solution of 2-iodoethanol (2 g, 0.0116 mol) and Imidazole (1.58 g, 0.0232 mol) in DCM (40 mL) was added tert-butyl-chloro-dimethyl-silane (1.9 g, 0.0126 mol) at 0° C. Reaction was warmed to room temperature and stirred for 4 hours. The reaction mixture was diluted with DCM (100 mL), washed with sat NaHCO₃ and brine, dried over Na₂SO₄ and concentrated under reduced pressure to get tert-butyl-(2-iodoethoxy)-dimethyl-silane C132 (2.5 g, 68%), ¹H NMR (400 MHz, Chloroform-d) δ 3.83 (t, J=6.8 Hz, 2H), 3.20 (t, J=6.8 Hz, 2H), 0.90 (s, 9H), 0.08 (s, 6H).

### Step 2: 1-[2-[tert-butyl(dimethyl)silyl]oxyethyl]pyrazole-4-carbaldehyde (S88)

To a solution of 1H-pyrazole-4-carbaldehyde (20 g, 208.1 mmol) and K₂CO₃ (115 g, 832.1 mmol) in MeCN (200 mL) was added tert-butyl-(2-iodoethoxy)-dimethyl-silane C132 (65 g, 227.1 mmol). Reaction was heated to 80° C. Reaction was stirred for 5 hours. Reaction was cooled to 50° C. and stirred for 16 hours. Reaction mixture was warmed, filtered, and solids were washed with MeCN (200 mL). Solids were discarded. Filtrate was concentrated. Residue was partitioned between EtOAc (400 mL) and water (400 mL). Organic layer was separated, washed with water (400 mL) and brine (400 mL), dried over MgSO₄, filtered, and concentrated. Purification by silica gel chromatography (800 g column, 0-80% EtOAC in hexane) afforded the product. 1-[2-[tert-butyl(dimethyl)silyl]oxyethyl]pyrazole-4-carbaldehyde S88 (46 g, 87%) as a pale yellow oil. ¹H NMR (300 MHz, Chloroform-d) δ 9.86 (s, 1H), 7.98 (s, 2H), 4.25 (dd, J=5.5, 4.5 Hz, 2H), 3.96 (dd, J=5.5, 4.5 Hz, 2H), 0.83 (s, 9H), −0.06 (s, 6H). LCMS m/z 255.14 [M+H]⁺.

### Preparation S89

### 1-[3-[tert-butyl(dimethyl)silyl]oxy-2-[[tert-butyl(dimethyl)silyl]oxymethyl]-2-methyl-propyl]pyrazole-4-carbaldehyde (S89)

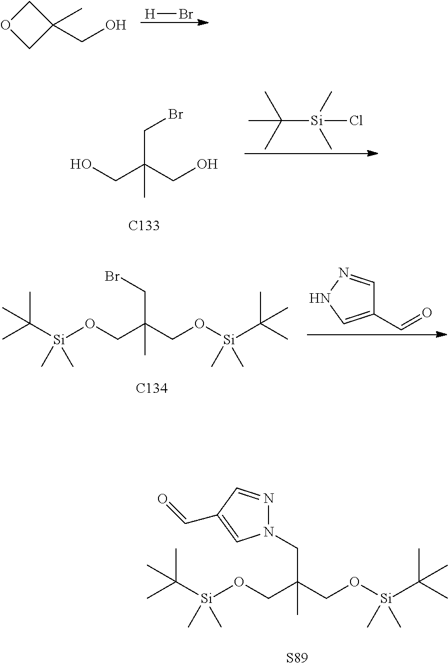

### Step 1: 2-(bromomethyl)-2-methyl-propane-1,3-diol (C133)

To a mixture of (3-methyloxetan-3-yl)methanol (10 mL, 100.3 mmol) in THF (70 mL) at 0° C. was added hydrogen bromide (14 mL of 48% w/w, 123.7 mmol). After stirring for 24 hours, the mixture was concentrated to a minimum volume, diluted in DCM/Methanol and the excess HBr was quenched with sat. sodium bicarbonate. The layers were split and the organic layer was dried with sodium sulfate, filtered, rinsed with methanol and concentrated to yield 2-(bromomethyl)-2-methyl-propane-1,3-diol C133 (13.6682 g, 74%) ¹H NMR (400 MHz, Methanol-d4) δ 3.47 (d, J=1.1 Hz, 6H), 0.96 (s, 3H).

### Step 2: [2-(bromomethyl)-3-[tert-butyl(dimethyl)silyl]oxy-2-methyl-propoxy]-tert-butyl-dimethyl-silane (C134)

To a mixture of 2-(bromomethyl)-2-methyl-propane-1,3-diol C133 (10 g, 54.09 mmol) in DCM (200 mL) was added imidazole (7.7 g, 113.1 mmol) followed by TBSCl (17 g, 112.8 mmol). After 5 min the mixture had precipitated a white crystalline solid. The mixture was filtered, rinsed with DCM, and concentrated. The mixture was diluted with heptane (25 mL) to further precipitate imidazole/imidazole HCl, filtered, and the solid was rinsed with additional heptane (10 mL). The mixture was concentrated, which precipitated additional solid. The mixture was diluted and concentrated twice more with heptane (50 mL) to afford [2-(bromomethyl)-3-[tert-butyl(dimethyl)silyl]oxy-2-methyl-propoxy]-tert-butyl-dimethyl-silane C134 (22246 mg, 100%) ¹H NMR (400 MHz, Chloroform-d) δ 3.44 (s, 4H), 3.40 (s, 2H), 0.94 (s, 3H), 0.89 (s, 18H), 0.04 (d, J=1.2 Hz, 12H).

### Step 3: 1-[3-[tert-butyl(dimethyl)silyl]oxy-2-[[tert-butyl(dimethyl)silyl]oxymethyl]-2-methyl-propyl]pyrazole-4-carbaldehyde (S89)

To a vial was added 1H-pyrazole-4-carbaldehyde (2 g, 20.81 mmol), dipotassium; carbonate (4 g, 28.94 mmol), and [2-(bromomethyl)-3-[tert-butyl(dimethyl)silyl]oxy-2-methyl-propoxy]-tert-butyl-dimethyl-silane C134 (9.5 g, 23.08 mmol) in DMF (20 mL). The mixture was heated to 130° C. After 3 hours the mixture was cooled to room temperature, diluted with water (100 mL) and heptane (100 mL). The layers were mixed, and the aqueous layer was washed with heptane (2×100 mL). The combined organic layer was washed with water (100 mL), brine (100 mL) and the organic layer was dried with sodium sulfate and concentrated. Purification by silica gel chromatography (Gradient: 0-60% EtOAc:Heptane) yielded the product 1-[3-[tert-butyl(dimethyl)silyl]oxy-2-[[tert-butyl(dimethyl)silyl]oxymethyl]-2-methyl-propyl]pyrazole-4-carbaldehyde S89 (2390 mg, 23%) ¹H NMR (400 MHz, Chloroform-d) δ 9.85 (s, 1H), 7.98-7.91 (m, 2H), 4.12 (s, 2H), 3.43-3.29 (m, 4H), 0.91 (s, 18H), 0.84 (s, 3H), 0.05 (d, J=0.6 Hz, 12H). LCMS m/z 427.31 [M+H]⁺.

### Preparation S90

### 2-[(2-hydroxy-1,1-dimethyl-ethyl)amino]pyrimidine-5-carbaldehyde (S90)

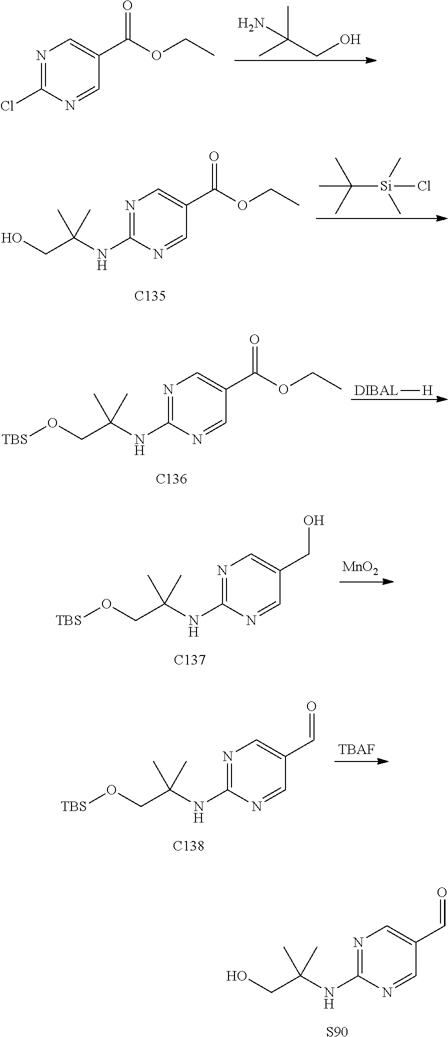

### Step 1: ethyl 2-[(2-hydroxy-1,1-dimethyl-ethyl)amino]pyrimidine-5-carboxylate (C135)

To a stirred solution of ethyl 2-chloropyrimidine-5-carboxylate (25 g, 0.1340 mol) in Ethanol (750 mL) was added 2-amino-2-methyl-propan-1-ol (14.333 g, 15.412 mL, 0.1608 mol) followed by DIPEA (34.637 g, 46.681 mL, 0.2680 mol) at room temperature. The reaction was stirred at 80° C. for 8 hours. The reaction was warmed to room temperature and concentrated under reduced pressure. Purification by silica gel chromatography (Eluent: 70% EtOAc in pet ether) afforded ethyl 2-[(2-hydroxy-1,1-dimethyl-ethyl)amino]pyrimidine-5-carboxylate C135 (18 g, 55%) ¹H NMR (400 MHz, DMSO-d6) δ 8.70 (s, 2H), 7.39 (s, 1H), 4.86 (t, J=6 Hz, 1H), 4.25 (q, J=6.8 Hz, 2H), 3.52 (d, J=6 Hz, 2H), 1.32 (s, 6H), 1.28 (t, J=6.8 Hz, 3H). LCMS m/z 240.27 [M+H]⁺.

### Step 2: ethyl 2-[[2-[tert-butyl(dimethyl)silyl]oxy-1,1-dimethyl-ethyl]amino]pyrimidine-5-carboxylate (C136)

To a stirred solution of ethyl 2-[(2-hydroxy-1,1-dimethyl-ethyl)amino]pyrimidine-5-carboxylate C135 (10 g, 0.0410 mol) and tert-butyl-chloro-dimethyl-silane (9.2694 g, 0.0615 mol) in DCM (500 mL) was added Imidazole (8.3735 g, 0.1230 mol) followed by DMAP (1.0018 g, 0.0082 mol) at room temperature and stirred for 16 h. Reaction was concentrated under reduced pressure. Crude was diluted with water (500 mL) and pentane (500 mL). The organic layer was separated, washed with water, dried over Na₂SO₄ and concentrated under reduced pressure to get ethyl 2-[[2-[tert-butyl(dimethyl)silyl]oxy-1,1-dimethyl-ethyl]amino]pyrimidine-5-carboxylate C136 (14.9 g, 100%) ¹H NMR (400 MHz, DMSO-d6) δ 8.70 (s, 2H), 7.46 (s, 1H), 4.25 (q, J=7.2 Hz, 2H), 3.77 (s, 2H), 1.30 (s, 6H), 1.28 (t, J=7.6 Hz, 3H), 0.82 (s, 9H), −0.06 (s, 6H). LCMS m/z 354.3 [M+H]⁺.

### Step 3: [2-[[2-[tert-butyl(dimethyl)silyl]oxy-1,1-dimethyl-ethyl]amino]pyrimidin-5-yl]methanol (C137)

To a stirred solution of ethyl 2-[[2-[tert-butyl(dimethyl)silyl]oxy-1,1-dimethyl-ethyl]amino]pyrimidine-5-carboxylate C136 (15 g, 0.0411 mol) in THF (600 mL), was added DIBAL-H (1M in Toluene) (205.50 mL of 1 M, 0.2055 mol) at −78° C. slowly under nitrogen balloon pressure. Reaction was stirred for 30 min −78° C. Stirred for 4 hours at room temperature. The reaction mixture was quenched with sat NH₄Cl (500 mL) at 0° C. and compound was extracted with EtOAc (2×500 mL). The organic layer was washed with 1N HCl (100 mL), brine, dried over Na₂SO₄ and concentrated under reduced pressure. Purification by silica gel chromatography (Eluent: 50% EtOAc in pet ether) afforded [2-[[2-[tert-butyl(dimethyl)silyl]oxy-1,1-dimethyl-ethyl]amino]pyrimidin-5-yl]methanol C137 (6 g, 46%) ¹H NMR (400 MHz, DMSO-d6) δ 8.19 (s, 2H), 6.25 (s, 1H), 4.99 (t, J=5.6 Hz, 1H), 4.27 (d, J=5.6 Hz, 2H), 3.71 (s, 2H), 1.30 (s, 6H), 0.84 (s, 9H), −0.03 (s, 6H). LCMS m/z 312.23 [M+H]⁺.

### Step 4: 2-[[2-[tert-butyl(dimethyl)silyl]oxy-1,1-dimethyl-ethyl]amino]pyrimidine-5-carbaldehyde (C138)

To a stirred solution of [2-[[2-[tert-butyl(dimethyl)silyl]oxy-1,1-dimethyl-ethyl]amino]pyrimidin-5-yl]methanol C137 (120 mg, 271.55 μmol) in DCM (10 mL) was added MnO₂ (851.98 mg, 0.0098 mol) at room temperature and stirred for 6 hours. Reaction was filtered through Celite®, and washed with DCM (10 mL). The filtrates were concentrated under reduced pressure to get 2-[[2-[tert-butyl(dimethyl)silyl]oxy-1,1-dimethyl-ethyl]amino]pyrimidine-5-carbaldehyde C138 (90 mg, 99%) ¹H NMR (400 MHz, DMSO-d6) δ 9.71 (s, 1H), 8.71 (d, J=11.6 Hz, 2H), 7.72 (s, 1H), 3.78 (s, 2H), 1.34 (s, 6H), 0.84 (s, 9H), −0.05 (s, 6H). LCMS m/z 310.22 [M+H]⁺.

### Step 5: 2-[(2-hydroxy-1,1-dimethyl-ethyl)amino]pyrimidine-5-carbaldehyde (S90)

To a stirred solution of 2-[[2-[tert-butyl(dimethyl)silyl]oxy-1,1-dimethyl-ethyl]amino]pyrimidine-5-carbaldehyde C138 (2.9 g, 0.0087 mol) in THF (20 mL) was added TBAF (1M in THF) (21.700 mL of 1 M, 0.0217 mol) at room temperature and stirred for 2 h. Reaction was diluted with EtOAc (100 mL), washed with brine solution, dried over Na₂SO₄ and concentrated under reduced pressure to get crude compound. Crude compound was washed with pentane and dried to get 2-[(2-hydroxy-1,1-dimethyl-ethyl)amino]pyrimidine-5-carbaldehyde S90 (1.47 g, 86%) ¹H NMR (400 MHz, DMSO-d6) δ 9.72 (s, 1H), 8.71 (d, J=13.2 Hz, 2H), 7.64 (s, 1H), 4.87 (t, J=6 Hz, 1H), 3.54 (d, J=6 Hz, 2H), 1.33 (s, 6H). LCMS m/z 196.35 [M+H]⁺.

### Preparation S91

### 4-chloro-6-methyl-pyrazolo[1,5-a]pyrazine-2-carbaldehyde (S91)

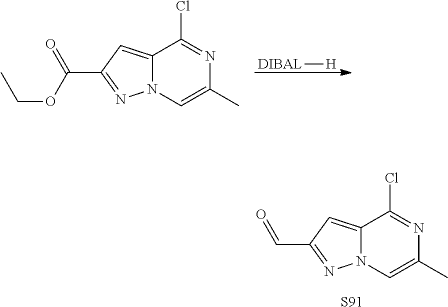

To an oven dried vial was added ethyl 4-chloro-6-methyl-pyrazolo[1,5-a]pyrazine-2-carboxylate (381 mg, 1.590 mmol), which was then dissolved in DCM (10 mL) under an argon atmosphere. This solution was cooled to −78° C. To this mixture was added diisobutylaluminum hydride (1.8 mL of 1 M, 1.800 mmol) solution in DCM slowly over 30 min. After stirring for 1 h, an additional portion of diisobutylaluminum hydride (2.4 mL of 1 M, 2.400 mmol) solution in DCM was added and the reaction was stirred for 60 min, and then quenched with MeOH (1 mL) followed by brine and diluted with DCM and stirred overnight. It was then diluted further with DCM and water and passed through a phase separator. The aqueous layer was extracted with DCM (3×30 mL), which also passed through a phase separator. The combined organic layer was concentrated in vacuo and the mixture was purified by silica gel chromatography (Eluent: 0-60% EtOAc in heptane) to afford 4-chloro-6-methyl-pyrazolo[1,5-a]pyrazine-2-carbaldehyde (237 mg, 76%). 1H NMR (300 MHz, Chloroform-d) δ 10.20 (s, 1H), 8.22-8.12 (m, 1H), 7.34 (d, J=1.0 Hz, 1H), 2.56 (d, J=1.1 Hz, 3H). LCMS m/z 195.99 [M+H]⁺.

### Preparation S92

### (2-chloropyrimidin-5-yl)methanol (S92)

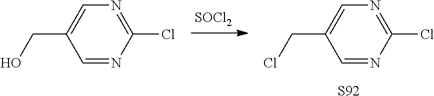

A 50 mL round bottom flask was charged with (2-chloropyrimidin-5-yl)methanol (1 g, 6.918 mmol) and thionyl chloride (12.5 mL, 171.4 mmol). To the suspension was added DMF (32.5 μL, 0.4197 mmol) and the resulting mixture was heated at reflux for approximately 5 h. The reaction was cooled to rt then concentrated in vacuo. It was then coevaporated with DCM (2×) and dried to constant mass under high vacuum to afford 2-chloro-5-(chloromethyl)pyrimidine (1.08 g, 92%) 1H NMR (300 MHz, Chloroform-d) δ 8.67 (s, 2H), 4.57 (d, J=0.6 Hz, 2H). LCMS m/z 162.95 [M+H]⁺.

### Preparation S93

### N-[5-(chloromethyl)-2-pyridyl]cyclopropanesulfonamide (S93)

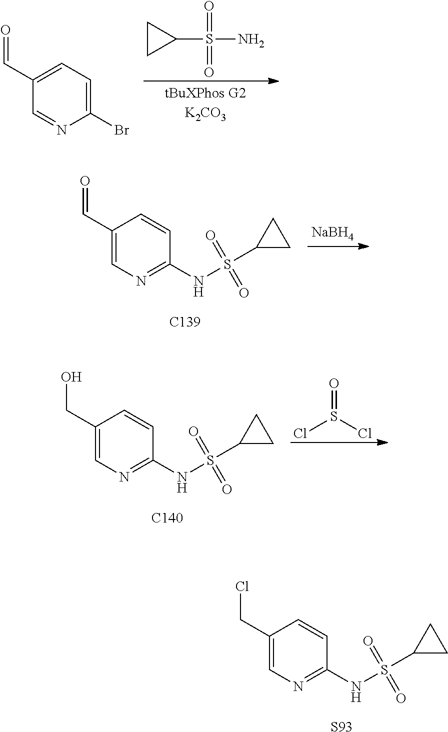

### Step 1: Synthesis of N-(5-formylpyridin-2-yl)cyclopropanesulfonamide (C139)

A mixture of 6-bromopyridine-3-carbaldehyde (70 mg, 0.3763 mmol), cyclopropanesulfonamide (137 mg, 1.131 mmol), K₂CO₃ (107 mg, 0.7742 mmol), and tBuXPhos G2 (15 mg, 0.01888 mmol) in 2-MeTHF (3.5 mL) was heated to 80° C. and stirred overnight. The reaction was cooled to rt and then diluted with EtOAc and 1 N HCl. The solution was extracted with EtOAc and washed with brine. The crude material was purified by silica gel chromatography (Gradient: 0-20% MeOH in DCM) to give the product N-(5-formylpyridin-2-yl)cyclopropanesulfonamide (46 mg, 27%). LCMS m/z 226.97 [M+H]⁺.

### Step 2: Synthesis of N-[5-(hydroxymethyl)-2-pyridyl]cyclopropanesulfonamide (C140)

N-(5-formylpyridin-2-yl)cyclopropanesulfonamide (46 mg, 0.20 mmol) was then dissolved in THF (1.9 mL) and NaBH₄ (28 mg, 0.7401 mmol) was added. The solution was stirred overnight. The reaction was washed with water, extracted with DCM, washed with HCl, and washed with brine. The aqueous layer was removed in vacuo. The product was redissolved in DCM and the aqueous salts were filtered off to give N-[5-(hydroxymethyl)-2-pyridyl]cyclopropanesulfonamide (19 mg, 57%). LCMS m/z 229.22 [M+H]⁺.

### Step 3: Synthesis of N-[5-(chloromethyl)-2-pyridyl]cyclopropanesulfonamide (S93)

N-[5-(hydroxymethyl)-2-pyridyl]cyclopropanesulfonamide (19 mg, 0.083 mmol) was dissolved in thionyl chloride (450 mg, 3.782 mmol) and the reaction was stirred at 60° C. for 2 h. The solvent was removed in vacuo and the crude was used without further purification. N-[5-(chloromethyl)-2-pyridyl]cyclopropanesulfonamide (20 mg, 51%). LCMS m/z 246.92 [M+H]⁺.

### Preparation S94

### 1-[tert-butyl(dimethyl)silyl]oxy-2-methyl-propan-2-amine (S94)

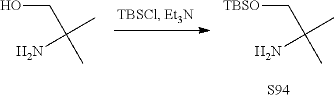

A 0° C. solution of 2-amino-2-methyl-propan-1-ol (2.5 g, 28.05 mmol) in DCM (85 mL) was treated with tert-butyl-chloro-dimethyl-silane (5 mL, 26.87 mmol) followed by addition of Et₃N (5.9 mL, 42.33 mmol). The resulting solution was stirred at rt overnight. The reaction was worked up by partitioning with water (2×). The organic layers were washed with brine, dried over anhydrous sodium sulfate, filtered and concentrated in vacuo to afford 1-[tert-butyl(dimethyl)silyl]oxy-2-methyl-propan-2-amine (3.61 g, 63%). 1H NMR (300 MHz, Chloroform-d) δ 3.20 (d, J=28.3 Hz, 2H), 1.00 (d, J=2.3 Hz, 4H), 0.88-0.83 (m, 9H), 0.80 (s, 2H), −0.01 (d, J=5.1 Hz, 6H).

### Preparation S95

### 2-(5-chloro-3-thienyl)ethanol (S95)

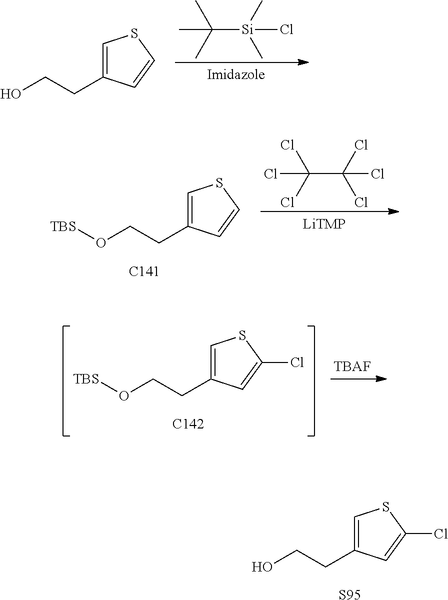

### Step 1. tert-butyl-dimethyl-[2-(3-thienyl)ethoxy]silane (C141)

To a solution of 2-(3-thienyl)ethanol (18 g, 140.4 mmol) in DMF (100 mL) was added imidazole (12 g, 176.3 mmol) and tert-butyl-chloro-dimethyl-silane (24 g, 159.2 mmol) sequentially. Exotherm was observed. The reaction mixture was stirred at room temperature for 3 hours. Reaction had stalled at 90% conversion. Reaction was diluted with MTBE (500 mL) and washed with water (200 mL), 0.5N HCl, (200 mL), water (200 mL), and brine (200 mL). The organic layer was dried, filtered and concentrated in vacuo. The organic layer was dissolved in heptane and passed through a silica gel plug; which was washed with 1-5% MTBE/Heptane. Solvent was removed to afford tert-butyl-dimethyl-[2-(3-thienyl)ethoxy]silane (C141) (34 g, 99%) ¹H NMR (400 MHz, Chloroform-d) δ 7.28-7.13 (m, 1H), 7.04-6.91 (m, 2H), 3.80 (t, J=6.9 Hz, 2H), 2.90-2.75 (m, 2H), 0.88 (s, 9H), −0.00 (s, 6H).

### Step 2. tert-butyl-[2-(5-chloro-3-thienyl)ethoxy]-dimethyl-silane (C142)

To a solution of 2,2,6,6-tetramethylpiperidine (36 mL, 213.3 mmol) in tetrahydrofuran (200 mL) cooled to 0° C.; was added a solution of hexyllithium (92 mL of 2.3 M, 211.6 mmol). Reaction was stirred for 30 minutes at −78° C. A solution of tert-butyl-dimethyl-[2-(3-thienyl)ethoxy]silane (34 g, 138.8 mmol) in THF (150 mL) was added to the reaction over 20 minutes. The reaction was stirred at −30° C. for 45 minutes. The reaction was cooled to −78° C. 1,1,1,2,2,2-hexachloroethane (54 g, 228.1 mmol) was added portion-wise. The reaction was warmed to room temperature and stirred overnight. The reaction was quenched with saturated ammonium chloride (125 mL), diluted with water (100 mL), extracted with EtOAc (500 mL), and back extracted with EtOAc (100 mL). The organic layer was washed with 0.5N HCl (200 mL), water (300 mL), and brine (200 mL). Organic layer was dried over sodium sulfate, filtered and concentrated to afford the crude product (C142).

### Step 3. 2-(5-chloro-3-thienyl)ethanol (S95)

To a solution of tert-butyl-[2-(5-chloro-3-thienyl)ethoxy]-dimethyl-silane (C142) (12.5 g, 42.89 mmol) in 2-Me-THF (120 mL) was added TBAF (63 mL of 1 M, 63.00 mmol) (solution in THF). Reaction was stirred at room temperature overnight. The reaction was partitioned between EtOAc (400 mL) and water (400 mL). The layers were separated and the organic layer was extracted with EtOAc (200 mL), dried over sodium sulfate, filtered, and concentrated in vacuo to remove solvent. Purification by silica gel chromatography (Gradient: 0-50% EtOAc in heptane) yielded the product 2-(5-chloro-3-thienyl)ethanol (S95) (4.5 g, 58%) ¹H NMR (300 MHz, Chloroform-d) δ 6.82 (d, J=0.9 Hz, 2H), 3.89-3.71 (m, 2H), 2.79 (t, J=6.4 Hz, 2H), 2.05 (s, 1H). LCMS m/z 162.91 [M+H]⁺.

### Preparation S96

### 2-[5-(trifluoromethyl)-3-thienyl]ethanol (S96)

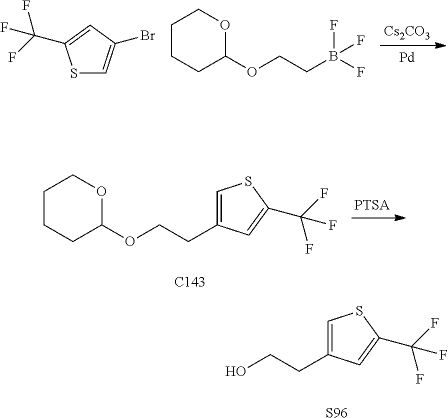

### Step 1. 2-[2-[5-(trifluoromethyl)-3-thienyl]ethoxy]tetrahydropyrane (C143)

To a mixture of 4-bromo-2-(trifluoromethyl)thiophene (9 g, 38.96 mmol), dicyclohexyl-[2-(2,6-diisopropoxyphenyl)phenyl]phosphane; methanesulfonate; N-methyl-2-phenyl-aniline; palladium(2+) (1.8 g, 2.117 mmol), and trifluoro(2-tetrahydropyran-2-yloxyethyl)boranuide (Potassium Ion (1)) (10 g, 42.36 mmol) was added Toluene (75 mL) and water (25 mL). Nitrogen was passed over the top of the reaction before addition of dicesium; carbonate (40 g, 122.8 mmol). A reflux condenser was added and the reaction was heated at 100° C. for 48 hours. The reaction was diluted with EtOAc (150 mL) and water (100 mL). The two layers were separated and the aqueous layered was extracted with EtOAc (100 mL). The combined organics were washed with brine, dried over sodium sulfate, filtered, and concentrated in vacuo. Purification by silica gel chromatography (Gradient: 0-20% EtOAc in heptane) yielded the product. 2-[2-[5-(trifluoromethyl)-3-thienyl]ethoxy]tetrahydropyran (C143) (9 g, 82%) ¹H NMR (300 MHz, Chloroform-d) δ 7.37 (t, J=1.3 Hz, 1H), 7.22 (d, J=1.5 Hz, 1H), 4.62 (dd, J=4.2, 2.8 Hz, 1H), 3.96 (dt, J=9.6, 6.7 Hz, 1H), 3.75 (ddd, J=11.3, 8.0, 3.4 Hz, 1H), 3.62 (dt, J=9.6, 6.5 Hz, 1H), 3.55-3.41 (m, 1H), 2.93 (t, J=6.6 Hz, 2H), 1.83 (ddd, J=14.2, 6.6, 3.4 Hz, 1H), 1.73 (td, J=9.0, 4.2 Hz, 1H), 1.66-1.50 (m, 4H).

### Step 2. 2-[5-(trifluoromethyl)-3-thienyl]ethanol (S96)

To a stirred solution of 2-[2-[5-(trifluoromethyl)-3-thienyl]ethoxy]tetrahydropyran (C143) (1.8 g, 6.100 mmol) in MeOH (25 mL) was added 4-methylbenzenesulfonic acid (Water (1)) (1.2 g, 6.309 mmol) at room temperature. The reaction mixture was stirred at room temperature for 1 hour. The reaction mixture was diluted with water (100 mL) and extracted with MTBE (2×100 mL). The organic layer was washed with dilute NaHCO₃ (10 mL NaHCO₃ and 10 mL water) and brine (10 mL), dried over sodium sulfate, filtered, and evaporated under vacuum to get crude compound. Purification by silica gel chromatography (Gradient: 0-30% EtOAc in heptane) yielded the product 2-[5-(trifluoromethyl)-3-thienyl]ethanol (S96) (820 mg, 69%) ¹H NMR (400 MHz, Chloroform-d) δ 7.35 (p, J=1.3 Hz, 1H), 7.23 (dt, J=1.7, 0.9 Hz, 1H), 3.85 (td, J=7.1, 6.5, 2.7 Hz, 2H), 2.87 (td, J=6.4, 0.8 Hz, 2H), 2.06 (d, J=4.3 Hz, 1H).

### Preparation S97

### 2-(5-ethyl-3-thienyl)ethanol (S97)

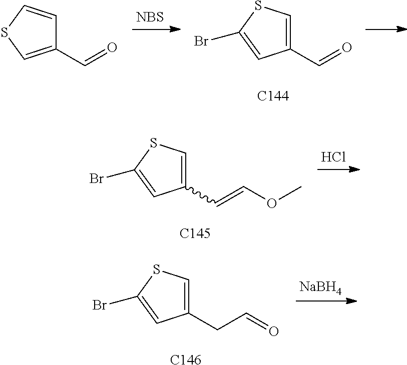

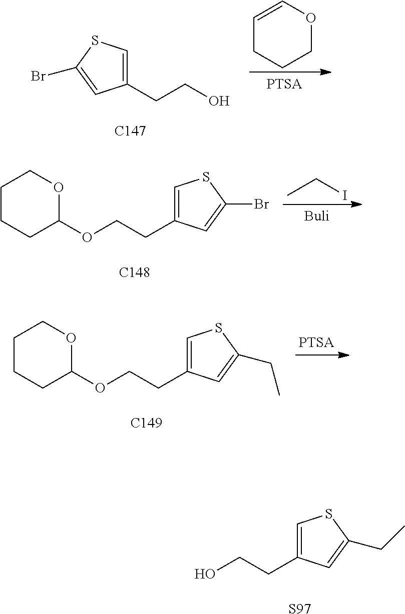

### Step 1. 5-bromothiophene-3-carbaldehyde (C144)

To a stirred solution of thiophene-3-carbaldehyde (50 g, 40.717 mL, 0.4458 mol) in DMF (500 mL) was added NBS (119.02 g, 0.6687 mol) at 0° C. The reaction mixture was stirred at room temperature for 16 hours. Reaction mixture was quenched with ice cold water (600 ml), and extracted with EtOAc (2×600 ml). Organic layer was dried on Na₂SO₄, filtered, and concentrated. Purification by silica gel chromatography (Gradient: 0-2% EtOAc in Pet Ether) yielded the product 5-bromothiophene-3-carbaldehyde (C144) (39.2 g, 44%) ¹H NMR (400 MHz, Chloroform-d) δ 9.77 (s, 1H), 7.99 (d, J 1.2 Hz, 1H), 7.505 (d, J 1.6 Hz, 1H).

### Step 2. 2-bromo-4-[(E)-2-methoxyvinyl]thiophene (C145)

To a stirred solution of (Methoxymethyl)triphenylphosphonium Chloride (115.1 g, 0.3358 mol) in Diethyl Ether (450.00 mL) at 0° C. was added Potassium tert-butoxide (1M in THF) (381 mL of 1 M, 0.3810 mol) drop-wise. The reaction was stirred at 0° C. for 1 hour. A solution of 5-bromothiophene-3-carbaldehyde (C144) (45 g, 0.2215 mol) in Diethyl Ether (90 mL) was added, and then the reaction mixture was stirred at room temperature for 30 minutes. Reaction mixture was quenched with NH₄Cl solution (900 mL) at 0° C., extracted with EtOAc (2×700 mL) and the organic layer was dried over Na₂SO₄, filtered and concentrated. Purification by silica gel chromatography (Eluent: Pet Ether) afforded the product, 2-bromo-4-[(E)-2-methoxyvinyl]thiophene (C145) (44.1 g, 82%) ¹H NMR (400 MHz, Chloroform-d) δ 7.25 (d, J 2 Hz, 1H), 7.18 (d, J 0.8 Hz, 1H), 7.00 (d, J=1.8 Hz, 1H), 6.91 (d, J 12.8 Hz, 1H), 6.97 (d, J 1.2 Hz, 1H), 6.05 (d, J 6.8 Hz, 1H), 5.72 (d, J 12.8 Hz, 1H), 5.22 (d, J 6.4 Hz, 1H), 3.77 (d, J 2.8 Hz, 3H), 3.64 (d, J 5.2 Hz, 3H). NMR shows a 1:1 mixture of E and Z isomers.

### Step 3. 2-(5-bromo-3-thienyl)acetaldehyde (C146)

To a stirred solution of 2-bromo-4-[(E)-2-methoxyvinyl]thiophene (C145) (14.1 g, 0.0602 mol) in 1,4-Dioxane (141.00 mL) was added HCl (4M in Dioxane) (60.200 mL of 4 M, 0.2408 mol) at 0° C. The reaction mixture was stirred at room temperature for 30 minutes. Reaction mixture was quenched with saturated NaHCO₃ at 0° C. and extracted with EtOAc. The organic layer was dried over Na₂SO₄, filtered and concentrated, to afford 2-(5-bromo-3-thienyl)acetaldehyde (C146) (13.1 g, 89%) ¹H NMR (400 MHz, Chloroform-d) δ 9.72 (t, J=2.4 Hz, 1H), 7.04 (s, 1H), 6.94 (d, J=1.2 Hz, 1H), 3.66 (d, J=1.6 Hz, 2H).

### Step 4. 2-(5-bromo-3-thienyl)ethanol (C147)

To a stirred solution of 2-(5-bromo-3-thienyl)acetaldehyde (C146) (38.5 g, 0.1524 mol) in MeOH (390 mL) was added NaBH₄ (13.3 g, 0.3515 mol) at 0° C. Reaction was stirred for 1 hour. The reaction mixture was quenched with ice water (400 mL) and concentrated in vacuo to remove the MeOH. The crude residue was diluted with water (500 ml) and extracted with EtOAc (3×300 ml). The separated organic layer was dried over Na₂SO₄, filtered and concentrated. Purification by column chromatography with neutral alumina (Eluent: 35% EtOAc in pet ether) afforded the product 2-(5-bromo-3-thienyl)ethanol (C147) (30.2 g, 84%) ¹H NMR (300 MHz, DMSO-d₆) δ 7.20 (t, J=0.9 Hz, 1H), 7.10 (d, J 1.2 Hz, 1H), 4.64 (q, J 5.2 Hz, 1H), 3.59-3.55 (m, 2H), 2.67 (t, J=6.8 Hz, 2H) as a pale yellow liquid.

### Step 5. 2-[2-(5-bromo-3-thienyl)ethoxy]tetrahydropyran (C148)

To a stirred solution of 2-(5-bromo-3-thienyl)ethanol (C147) (8 g, 0.0328 mol) in THF (80. mL) was added 3,4-Dihydro-2H-pyran (3.7696 g, 3.8 mL, 0.0448 mol) and PTSA (259 mg, 0.0015 mol) at room temperature. Then reaction mixture was stirred at room temperature for 16 hours. The reaction mixture was quenched with saturated aq. K₂CO₃ (300 mL), and extracted with EtOAc (2×600 mL). The organic layer was dried over Na₂SO₄, filtered, and concentrated. Purification by silica gel chromatography (Gradient: 0-5% EtOAc in Pet Ether) yielded the product 2-[2-(5-bromo-3-thienyl)ethoxy]tetrahydropyran (10.1 g, 90%) ¹H NMR (400 MHz, Chloroform-d) δ 6.95 (d, J=1.6 Hz, 1H), 6.92 (d, J=0.8, 1H), 4.59 (t, J=2.8 Hz, 1H), 3.94-3.74 (m, 2H), 3.60-3.46 (m, 2H), 2.85 (q, J=6.4 Hz, 2H), 1.80-1.61 (m, 6H). LCMS m/z 291.03 [M+H]⁺.

### Step 6. 2-[2-(5-ethyl-3-thienyl)ethoxy]tetrahydropyran (C149)

To a stirred solution of 2-[2-(5-bromotetrahydrothiophen-3-yl)ethoxy]tetrahydropyran (C148) (25 g, 0.0719 mol) in THF (250.00 mL) was added n-BuLi (2.5 M in Hexane) (46.1 mL of 2.5 M, 0.1153 mol) at −76° C. Reaction was stirred for 1 hour. Ethyl iodide (24.832 g, 12.8 mL, 0.1592 mol) was added at −76° C. Then reaction temperature was slowly increased to room temperature, and was then stirred for 16 hours. The reaction mixture was quenched with NH₄Cl solution (500 mL), and extracted with EtOAc (2×300 mL). Organic layer was dried over Na₂SO₄, filtered and concentrated. Purification by silica gel chromatography (Gradient: 0-3% EtOAc in Pet Ether) yielded the product 2-[2-(5-ethyl-3-thienyl)ethoxy]tetrahydropyran (C149) (13.2 g, 59%) LCMS m/z 241.21 [M+H]⁺.

### Step 7. 2-(5-ethyl-3-thienyl)ethanol (S97)

To a stirred solution of 2-[2-(5-ethyl-3-thienyl)ethoxy]tetrahydropyran (C149) (4.4 g, 0.0142 mol) in MeOH (44.000 mL) was added PTSA (3.0 g, 0.0174 mol) at room temperature and the reaction was stirred for 2 hours. Reaction mixture was quenched with saturated NaHCO₃ solution (150 mL), extracted with EtOAc (2×150 mL), dried over Na₂SO₄, filtered, and concentrated. Purification by column chromatography with neutral alumina (Eluent: 10% EtOAc in pet ether) afforded the product 2-(5-ethyl-3-thienyl)ethanol (S97) (1.1 g, 45%) ¹H NMR (400 MHz, DMSO-d₆) δ 6.90 (d, J=1.2 Hz, 1H), 6.71 (d, J=1.2 Hz, 1H), 4.62-4.58 (m, 1H), 3.59-3.55 (m, 2H), 2.77-2.71 (m, 2H), 2.64 (t, J=7.2, 2H), 1.22-1.85 (m, 3H).

### Preparation S98

### 2-(5-ethyl-2-thienyl)ethanol (S98)

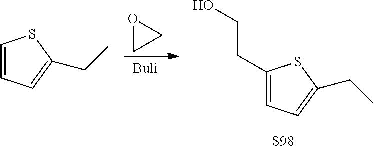

### Step 1. 2-(5-ethyl-2-thienyl)ethanol (S98)

To a solution of 2-ethylthiophene (54 g, 466.9 mmol) in anhydrous THF (1 L) at 0° C. was added n-BuLi in hexane (255 mL of 2.2 M, 561.0 mmol) over 45 minutes. A light yellow/orange solution resulted. The temperature range during the addition was 0-10° C. The mixture was stirred at room temperature for 30 minutes. After cooling to 0° C. a solution of ethylene-oxide (200 mL of 2.9 M, 580.0 mmol) was added over 30 minutes. The reaction was stirred at 0° C. for 2 hours and then was warmed to room temperature. Reaction mixture was quenched with water (700 mL) & saturated NH₄Cl (200 mL) and the THF was evaporated. The product was extracted with EtOAc (1×400 mL; 2×150 mL). The organic layer was dried over anhydrous sodium sulfate, filtered, and concentrated in vacuo. The crude product was dried in vacuo. The organic layer was passed through a silica gel plug washing with DCM (1000 mL), 80% EtOAc/Heptane (2×200 mL) and DCM (2×250 mL) to afford 2-(5-ethyl-2-thienyl)ethanol (S98) (71.25 g, 93%) ¹H NMR (300 MHz, Chloroform-d) δ 6.69 (dt, J=3.4, 0.9 Hz, 1H), 6.64 (dt, J=3.3, 1.0 Hz, 1H), 3.84 (t, J=6.3 Hz, 2H), 3.08-2.97 (m, 2H), 2.82 (qd, J=7.5, 1.0 Hz, 2H), 1.31 (t, J=7.5 Hz, 4H).

### Preparation S99

### 2-[5-(trifluoromethyl)-2-thienyl]ethanol (S99)

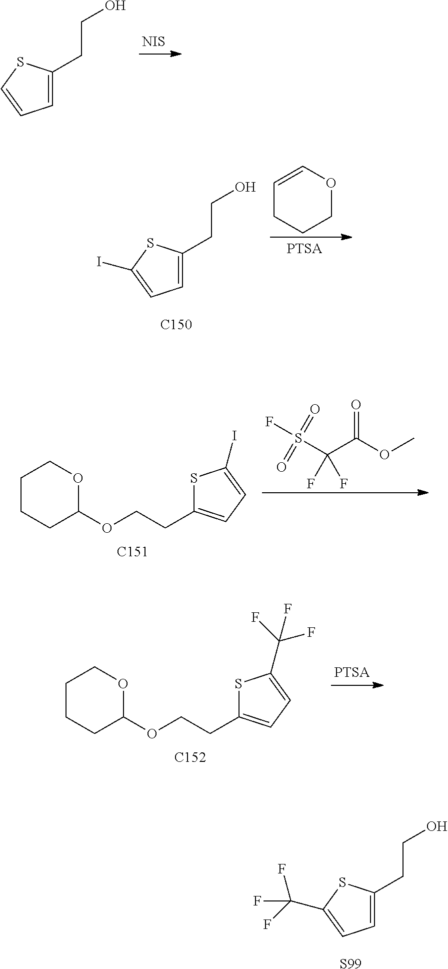

### Step 1: 2-(5-iodo-2-thienyl)ethanol (C150)

To a stirred solution of NIS (104.83 g, 0.4680 mol) in DCM (1000 mL) was added 2-(2-thienyl)ethanol (50 g, 0.3900 mol) at 0° C. Reaction was warmed to room temperature and stirred for 16 hours. The reaction mixture was diluted with DCM (500 mL), washed with sat sodium thiosulphate, brine, dried over Na₂SO₄ and concentrated in vacuo. Purification by column chromatography (Eluent: 20% EtOAc in pet ether) afforded the product 2-(5-iodo-2-thienyl)ethanol (C150) (62 g, 56%) ¹H NMR (400 MHz, Chloroform-d) δ 7.08 (d, J 3.6 Hz, 1H), 6.57-6.56 (m, 1H), 3.82 (q, J 6 Hz, 2H), 3.05 (q, J 6.4 Hz, 2H). LCMS m/z 254.89 [M+H]⁺.

### Step 2: 2-[2-(5-iodo-2-thienyl)ethoxy]tetrahydropyran (C151)

To a stirred solution of 2-(5-iodo-2-thienyl)ethanol (C150) (15 g, 0.0525 mol) and 3,4-dihydro-2H-pyran (6.6284 g, 0.0788 mol) in THF (60 mL) was added PTSA (1.3604 g, 1.2714 mL, 0.0079 mol) at room temperature. Reaction was stirred for 16 hours under argon balloon pressure. The reaction mixture was concentrated under reduced pressure. Purification by silica gel chromatography (Eluent: 5% EtOAc in Pet Ether) yielded the product 2-[2-(5-iodo-2 thienyl)ethoxy]tetrahydropyran (C151) (12.8 g, 68%), ¹H NMR (400 MHz, DMSO-d₆) δ 7.14 (d, J 3.6 Hz, 1H), 6.64 (d, J 3.6 Hz, 1H), 4.59 (t, J 3.6 Hz, 1H), 3.80-3.76 (m, 1H), 3.74-3.67 (m, 1H), 3.54-3.50 (m, 1H), 3.48-3.41 (m, 1H), 3.03 (t, J 6 Hz, 2H), 1.75-1.69 (m, 1H), 1.61-1.59 (m, 1H), 1.51-1.42 (m, 4H).

### Step 3: 2-[2-[5-(trifluoromethyl)-2-thienyl]ethoxy]tetrahydropyran (C152)

To a stirred solution of 2-[2-(5-iodo-2-thienyl)ethoxy]tetrahydropyran (C151) (10 g, 0.0219 mol) and methyl 2,2-difluoro-2-fluorosulfonyl-acetate (12.63 g, 0.0657 mol) in DMF (40 mL) was added Copper(I) bromide dimethyl sulfide complex 99% (2.241 g, 0.0109 mol). Reaction was stirred at 100° C. for 16 hours. The reaction was warmed to room temperature, diluted with EtOAc (100 mL), filtered, and washed with EtOAc (50 mL). The filtrates were washed with chilled brine solution, dried over Na₂SO₄, and concentrated under reduced pressure. Purification by column chromatography with neutral alumina (Eluent: 5% EtOAc in pet ether) afforded the product (C152) 2-[2-[5-(trifluoromethyl)-2-thienyl]ethoxy]tetrahydropyran (2.9 g, 41%) ¹H NMR (400 MHz, Chloroform-d) δ 7.25 (s, 1H), 6.82-6.81 (m, 1H), 4.63 (t, J 3.6 Hz, 1H), 4.00-3.95 (m, 1H), 3.78-3.75 (m, 1H), 3.64-3.58 (m, 1H), 3.51-3.48 (m, 1H), 3.12 (d, J 6.4 Hz, 2H), 1.90-1.80 (m, 1H), 1.73-1.64 (m, 1H), 1.65-1.51 (m, 4H).

### Step 4: 2-[5-(trifluoromethyl)-2-thienyl]ethanol (S99)

To a stirred solution of 2-[2-[5-(trifluoromethyl)-2-thienyl]ethoxy]tetrahydropyran (C152) (5.8 g, 0.0170 mol) in MeOH (100 mL) was added PTSA (2.93 g, 0.0170 mol) at room temperature. Reaction was stirred for 16 hours. The reaction mixture was concentrated under reduced pressure. Purification by column chromatography with neutral alumina (Eluent: 10% EtOAc in pet ether) afforded the product 2-[5-(trifluoromethyl)-2-thienyl]ethanol (S99) (2.3 g, 61%) ¹H NMR (400 MHz, DMSO-d₆) δ 7.52-7.51 (m, 1H), 6.99-6.98 (m, 1H), 4.92 (t, J 4.8 Hz, 1H), 3.65-3.61 (m, 2H), 2.98 (t, J 6 Hz, 2H). ¹⁹F NMR (376.22 MHz, DMSO-d6) δ−53.53 (s, 3F).

### Preparation S100

### 2-[5-(trifluoromethyl)-2-thienyl]propan-1-ol (S100)

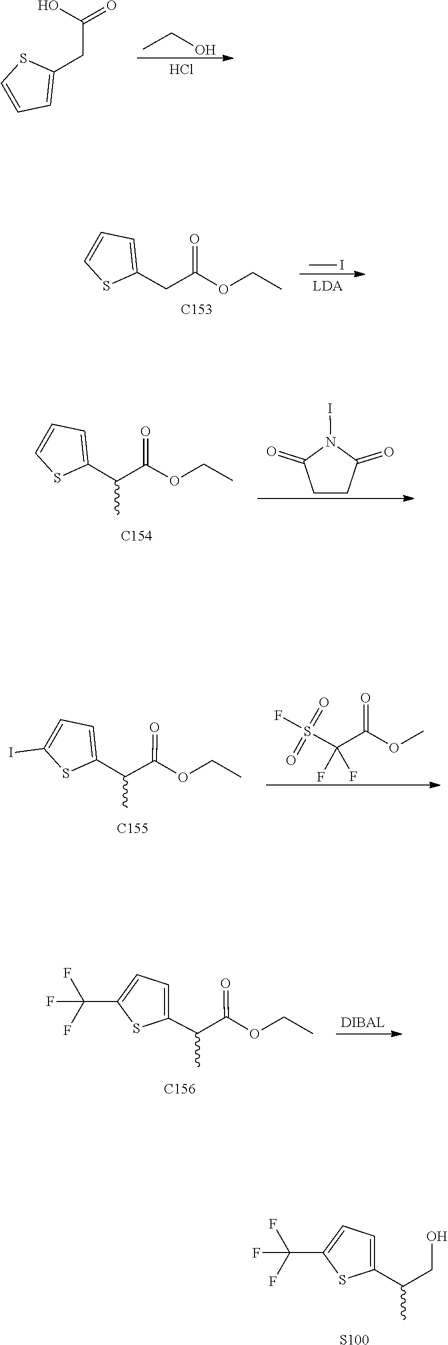

### Step 1: ethyl 2-(2-thienyl)acetateethanol (C153)

To a stirred solution of 2-(2-thienyl)acetic acid (100 g, 703.35 mmol) in ethanol (2000 mL) was added HCl (Aqueous) (50 mL of 36% w/v, 493.68 mmol) at room temperature. The reaction mixture was stirred for 12 hours at 70° C. The mixture was concentrated and the resulting crude was diluted with EtOAc (1000 ml), washed with 5% Na₂CO₃ aqueous solution (3×200 ml), and brine (200 ml). The organic layer was dried and concentrated to afford desired product, ethyl 2-(2-thienyl)acetate (C153) (100 g, 82%) ¹H NMR (Chloroform-d, 400 MHz): δ=7.222-7.206 (dd, J=1.2 Hz, J=3.6 Hz, 1H), 6.969-6.948 (m, 2H), 4.213-4.160 (q, J=7.2 Hz, 2H), 3.828 (s, 2H), 1.295-1.259 (t, J=7.2 Hz, 3H). LCMS m/z 171.26 [M+H]⁺.

### Step 2: ethyl 2-(2-thienyl)propanoate (C154)

To a solution of ethyl 2-(2-thienyl)acetate (C153) (1.36 g, 7.989 mmol) in THF (20 mL) at −78° C. was added (diisopropylamino)lithium (8 mL of 1 M, 8.000 mmol). After 15 minutes MeI (500 μL, 8.032 mmol) was added and the reaction mixture stirred at −78° C. for 2 hrs. The reaction was quenched with saturated NH₄Cl (50 mL) and extracted with EtOAc. The organic layer was dried and concentrated to an oil, which was purified by silica chromatography (Gradient: 0 to 25% EtOAc in heptane) to afford the product, ethyl 2-(2-thienyl)propanoate (C20) (1.04 g, 71%) ¹H NMR (300 MHz, Chloroform-d) δ 7.25-7.17 (m, 1H), 7.02-6.93 (m, 2H), 4.18 (d, J=7.2 Hz, 2H), 4.02 (q, J=7.1 Hz, 1H), 1.60 (d, J=7.2 Hz, 3H), 1.28 (t, J=7.1 Hz, 3H).

### Step 3: ethyl 2-(5-iodo-2-thienyl)propanoate (C155)

To a stirred solution of ethyl 2-(2-thienyl)propanoate C154 (35 g, 143.99 mmol) in Acetic acid (350 mL) was added N-Iodosuccinimide (38.875 g, 172.79 mmol). The reaction mixture was stirred for one hour at 100° C. The mixture was concentrated and the resulting crude was diluted with EtOAc (700 ml), washed with water (300 ml), saturated sodium bicarbonate solution (300 ml), saturated sodium thiosulfate solution (300 ml), and brine solution (250 ml) sequentially. The organic layer was dried over Na₂SO₄, filtered, and concentrated to afford crude product. Purification by silica gel chromatography (Eluent: 3% EtOAc in Pet Ether) yielded the product ethyl 2-(5-iodo-2-thienyl)propanoate (C155) (30 g, 42%) ¹H NMR (Chloroform-d, 400 MHz): δ=7.08 (d, J=4 Hz, 1H), 6.62 (d, J=4 Hz, 1H), 4.191-4.130 (m, 2H), 3.979-3.925 (m, 1H), 1.552-1.513 (m, 3H), 1.277-1.240 (m, 3H). LCMS m/z 309.9 [M+H]⁺.

### Step 4: ethyl 2-[5-(trifluoromethyl)-2-thienyl]propanoate (C156)

To a stirred solution of ethyl 2-(5-iodo-2-thienyl)propanoate (C155) (5 g, 9.9629 mmol) and Methyl 2,2-difluoro-2-(fluorosulfonyl)acetate (9.5700 g, 49.814 mmol) in DMF (50 mL) was added CuI (2.2768 g, 11.955 mmol) under nitrogen atmosphere. The reaction mixture was stirred for 12 hours at 100° C. The mixture was filtered through Celite® and the Celite® pad was washed with Diethyl Ether (2×100 ml). Filtrated was quenched with cold water (100 ml). The two layers were separated and the aqueous layer was extracted with diethyl ether (2×50 ml). The total organic layer was washed with brine (30 ml), dried, and concentrated. Purification by silica gel chromatography (Eluent: 3% EtOAc in Pet Ether) yielded the product ethyl 2-[5-(trifluoromethyl)-2-thienyl]propanoate (C156) (2 g, 58%) ¹H NMR (Chloroform-d, 400 MHz): δ=7.290-7.261 (m, 1H), 6.918-6.903 (m, 1H), 4.213-4.147 (m, 2H), 3.996-3.961 (m, 1H), 1.565-1.525 (m, 3H), 1.277-1.267 (m, 3H). LCMS m/z 252.1 [M].

### Step 5: 2-[5-(trifluoromethyl)-2-thienyl]propan-1-ol (S100)

To a stirred solution of ethyl 2-[5-(trifluoromethyl)-2-thienyl]propanoate (12 g, 41.701 mmol) in THF (250 mL) was added DIBAL-H (35.584 mL of 25% w/v, 62.552 mmol) drop-wise at 0° C. The reaction mixture was stirred for 2 hours at 0° C. The mixture was slowly quenched with saturated NH₄Cl solution (300 ml) at 0° C. and the suspension was filtered through Celite® and the Celite® pad was washed with EtOAc (2×200 ml). The filtrate was separated into layers. The aqueous layer was extracted with EtOAc (2×200 ml). The total organic layer was washed with brine (200 ml), dried over sodium sulfate and concentrated. Purification by silica gel chromatography (Eluent: 3% EtOAc in Pet Ether) yielded crude product. The crude compound was further purified by achiral SFC purification to give racemic 2-[5-(trifluoromethyl)-2-thienyl]propan-1-ol (3.64 g) ¹H NMR (400 MHz, Chloroform-d) δ 7.52 (m, 1H), 7.00 (m, 1H), 4.97 (t, J 5.6 Hz, 1H), 3.51 (t, J 6.0 Hz, 2H) 3.17 (m, 1H), 1.27 (d, J=6.8 Hz, 3H). LCMS m/z 210.0 [M].

### Preparation S101

### 2-methyl-2-[5-(trifluoromethyl)-2-thienyl]propan-1-ol (S101)

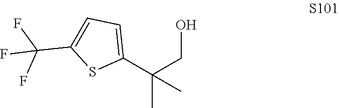

S8 was obtained during SFC purification of S100 as a side product due to over alkylation in step 2.

### Preparation S102, S103, and S104

### 2-methyl-2-[5-(chloro)-2-thienyl]propan-1-ol (S102)

### 2-[5-(chloro)-2-thienyl]propan-1-ol (S103 ENANT-1, S104 ENANT-2)

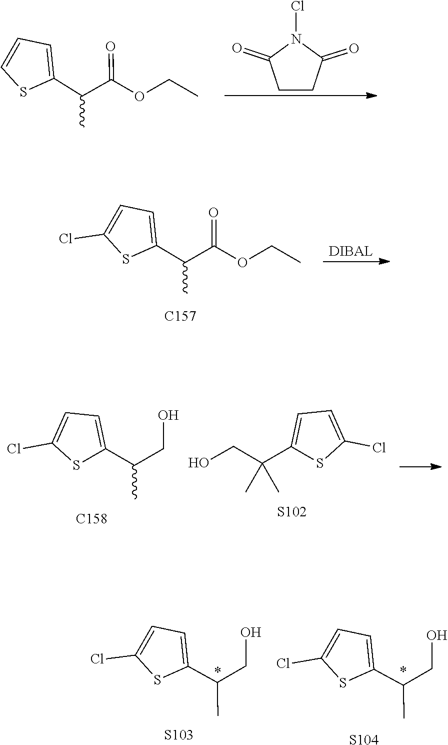

### Step 1: ethyl 2-(5-chloro-2-thienyl)propanoate (C157)

To a stirred solution of ethyl 2-(2-thienyl)propanoate (1 g, 4.1139 mmol) in Acetic acid (10 mL) was added N-Chlorosuccinimide (549.34 mg, 4.1139 mmol). The reaction mixture was stirred for 1 hour at 100° C. The mixture was concentrated and the resulting crude was diluted with EtOAc (25 ml), washed with water (10 ml), saturated sodium bicarbonate solution (10 ml), saturated sodium thiosulfate solution (10 ml), and brine solution (10 ml). The organic layer was dried over Na₂SO₄, filtered, and concentrated to afford crude product. Purification by silica gel chromatography (Eluent: 3% EtOAc in Pet Ether) yielded the product ethyl 2-(5-chloro-2-thienyl)propanoate (C157) (700 mg, 60%) ¹H NMR (Chloroform-d, 400 MHz): δ=6.751-6.731 (m, 1H), 6.712-6.697 (m, 1H), 4.195-4.139 (m, 2H), 3.876-3.729 (q, J=6.4 Hz, 1H), 1.545-1.527 (t, J=2.8 Hz, 3H), 1.300-1.221 (m, 3H). LCMS m/z 218.0 [M].

### Step 2: 2-(5-chloro-2-thienyl)-2-methyl-propan-1-ol and 2-(5-chloro-2-thienyl)propan-1-ol (S102) and (C158)

To a stirred solution of ethyl 2-(5-chloro-2-thienyl)propanoate (C157) (25 g, 86.877 mmol) in THF (500 mL) was added DIBAL-H (74.135 mL of 25% w/v, 130.32 mmol) drop-wise at 0° C. The reaction mixture was stirred for 2 hours at 0° C. The mixture was slowly quenched with saturated NH₄Cl solution (300 ml) at 0° C. and the suspension was filtered through Celite® and the Celite® pad was washed with EtOAc (2×200 ml). The filtrate was separated into two layers. The aqueous layer was extracted with EtOAc (2×200 ml). The combined organic layer was washed with brine (200 ml), dried over sodium sulfate and concentrated. Purification by silica gel chromatography (Eluent: 3% EtOAc in Pet Ether) yielded (S102) 2-(5-chloro-2-thienyl)-2-methyl-propan-1-ol (410 mg, 2%) ¹H NMR (Chloroform-d, 400 MHz): δ=6.762-6.752 (d, J=4 Hz, 1H), 6.671-6.649 (t, J=4 Hz, 1H), 3.540-3.523 (d, J 6.8 Hz, 2H), 1.466-1.433 (t, J=6.8 Hz, 1H), 1.339 (s, 6H). LCMS m/z 190.0 [M]; and 2-(5-chloro-2-thienyl)propan-1-ol (C158) (12 g, 72%) ¹H NMR (Chloroform-d, 400 MHz): δ=6.763-6.754 (d, J=3.6 Hz, 1H), 6.663-6.652 (dd, J=4.4 Hz, 1H), 3.712-3.606 (m, 2H), 3.154-3.103 (m, 1H), 1.570-1.522 (m, 1H), 1.338-1.306 (t, J=6 Hz, 3H). LCMS m/z 176.0 [M].

### Step 3: 2-(5-chloro-2-thienyl)propan-1-ol (S103) and (S104)

The Racemic compound 2-(5-chloro-2-thienyl)propan-1-ol (C158) (12 g, 62.492 mmol) was purified into two single enantiomers by using SFC purification to afford:

2-(5-chloro-2-thienyl)propan-1-ol (S103) (4 g, 35%) ¹H NMR (Chloroform-d, 400 MHz): δ=6.762-6.753 (d, J=3.6 Hz, 1H), 6.662-6.651 (dd, J=3.6 Hz, 1H), 3.731-3.610 (m, 2H), 3.170-3.103 (m, 1H), 1.523-1.492 (t, J=5.2 Hz, 1H), 1.324-1.307 (d, J=6.8 Hz, 3H). LCMS m/z 176.0 [M]; and 2-(5-chloro-2-thienyl)propan-1-ol (S104) (3.75 g, 34%) ¹H NMR (Chloroform-d, 400 MHz): δ=6.763-6.753 (d, J=4 Hz, 1H), 6.662-6.651 (dd, J=3.6 Hz, 1H), 3.731-3.611 (m, 2H), 3.154-3.104 (q, J=6.8 Hz, 1H), 1.511-1.480 (t, J=5.6 Hz, 1H), 1.325-1.307 (d, J=7.2 Hz, 3H). LCMS m/z 176.0 [M].

### Preparation S105 and S106

### 2-(5-ethyl-2-thienyl)propan-1-ol (S105 ENANT-1) and (S106 ENANT-2)

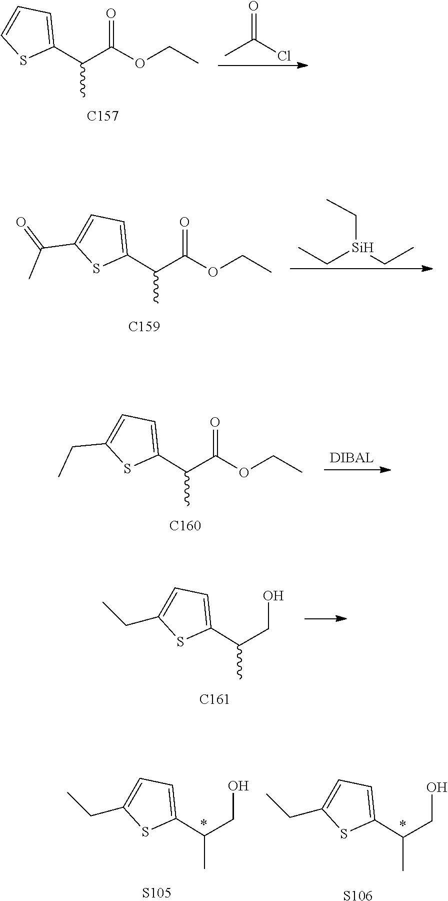

### Step 1: ethyl 2-(5-acetyl-2-thienyl)propanoate (C159)

To a stirred solution of ethyl 2-(2-thienyl)propanoate (C159) (80 g, 336.92 mmol) in DCM (1500 mL) was added Acetyl chloride (39.671 g, 35.934 mL, 505.38 mmol) drop-wise at 0° C., followed by addition of AlCl₃ (67.388 g, 505.38 mmol) at 0° C. The reaction mixture was stirred for 2 hours at 0° C. The mixture was slowly quenched with ice water (1000 ml), the two layers were separated and the aqueous layer was further extracted with DCM (2×500 ml). The total organic layer was washed with brine (500 ml), dried over sodium sulfate and purification by silica gel chromatography (Gradient: 0-5% EtOAc in Pet Ether) yielded the product ethyl 2-(5-acetyl-2-thienyl)propanoate (C159) (60 g, 73%) ¹H NMR (Chloroform-d, 400 MHz): δ=7.559-7.543 (t, J=4.0 Hz, 1H), 6.991-6.980 (m, 1H), 4.200-4.140 (m, 2H), 4.015-3.962 (q, J=7.2 Hz, 1H), 2.525 (s, 3H), 1.603-1.585 (d, J 7.2 Hz, 3H), 1.277-1.230 (m, 3H). LCMS m/z 227.1 [M+H]⁺.

### Step 2: ethyl 2-(5-ethyl-2-thienyl)propanoate (C160)

To a stirred solution of ethyl 2-(5-acetyl-2-thienyl)propanoate C159 (60 g, 245.79 mmol) in TFA (400 mL) was added Triethyl silane (42.870 g, 58.887 mL, 368.69 mmol) drop-wise at 0° C. The reaction mixture was stirred for 4 hours at room temperature. The reaction was concentrated and quenched with ice water (500 ml) and extracted with EtOAc (3×500 ml). The total organics were washed with brine (250 ml), dried over sodium sulfate, and concentrated to afford crude product. Purification by silica gel chromatography (Gradient: 0-3% EtOAc in Pet Ether) yielded the product ethyl 2-(5-ethyl-2-thienyl)propanoate (C160) (50 g, 82%) ¹H NMR (Chloroform-d, 400 MHz): δ=6.735-6.725 (dd, J=3.6 Hz, 1H), 6.615-6.601 (m, 1H), 4.186-4.127 (m, 2H), 3.930-3.876 (q, J=7.2 Hz, 1H), 2.824-2.766 (m, 2H), 1.550-1.532 (d, J=7.2 Hz, 3H) 1.303-1.229 (m, 6H). LCMS m/z 213.2 [M+H]⁺.

### Step 3: 2-(5-ethyl-2-thienyl)propan-1-ol (C₁₆₁)

To a stirred solution of ethyl 2-(5-ethyl-2-thienyl)propanoate (C160) (50 g, 200.18 mmol) in THF (1000 mL) was added DIBAL-H (25% in Toluene) (227.75 mL of 25% w/v, 400.36 mmol) drop-wise at 0° C. The reaction mixture was stirred for 2 hours at 0° C. The mixture was slowly quenched with saturated NH₄Cl solution (500 ml) at 0° C. and extracted with EtOAc (2×500 ml). The total organic layer was washed with brine (250 ml), dried over sodium sulfate and concentrated. Purification by silica gel chromatography (Gradient: 0-5% EtOAc in Pet Ether) yielded the product, 2-(5-ethyl-2-thienyl)propan-1-ol (C161) (31 g, 89%) ¹H NMR (Chloroform-d, 400 MHz): δ=6.686-6.677 (d, J=3.6 Hz, 1H), 6.635-6.621 (m, 1H), 3.724-3.601 (m, 2H), 3.184-3.134 (q, J=6.8 Hz, 1H), 2.831-2.772 (m, 2H), 1.609-1.548 (m, 1H), 1.350-1.276 (m, 6H). LCMS m/z 171.02 [M+H]⁺.

### Step 4: 2-(5-ethyl-2-thienyl)propan-1-ol (S105) and (S106)

The Racemic compound 2-(5-ethyl-2-thienyl)propan-1-ol (31 g, 178.06 mmol) was purified into two single enantiomers by using SFC purification. 2-(5-ethyl-2-thienyl)propan-1-ol (S105) (13.45 g, 43%) ¹H NMR (Chloroform-d, 400 MHz): δ=6.686-6.678 (d, J=3.2 Hz, 1H), 6.632-6.624 (d, J=3.2 Hz, 1H), 3.730-3.607 (m, 2H), 3.187-3.137 (q, J=6.8 Hz, 1H), 2.831-2.775 (m, 2H), 1.535-1.470 (m, 1H), 1.352-1.271 (m, 6H). LCMS m/z 171.1 [M+H]⁺; and 2-(5-ethyl-2-thienyl)propan-1-ol (S106) (11.35 g, 37%)¹H NMR (Chloroform-d, 400 MHz): δ=6.686-6.677 (d, J=3.6 Hz, 1H), 6.632-6.623 (d, J=3.6 Hz, 1H), 3.729-3.606 (m, 2H), 3.203-3.119 (m, 1H), 2.830-2.773 (q, J=7.6 Hz, 2H), 1.542-1.478 (m, 1H), 1.329-1.271 (m, 6H). LCMS m/z 171.1 [M+H]⁺.

### Preparation S107

### 2-(5-methyl-3-thienyl)ethanol (S107)

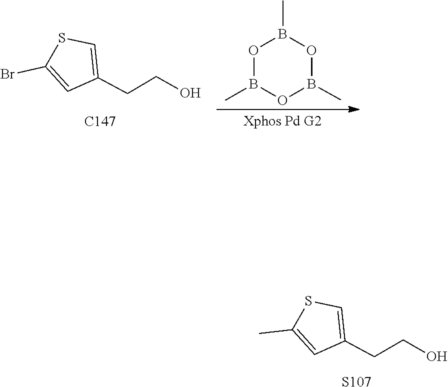

### Step 1: 2-(5-methyl-3-thienyl)ethanol (S107)

To a stirred solution of 2-(5-bromo-3-thienyl)ethanol (C147) (2.5 g, 0.0098 mol) in 1,4-Dioxane (16.000 mL) was added K₂CO₃ (4.9 g, 0.0355 mol) at room temperature in a sealed tube. Reaction mixture was degassed with argon gas for 10 minutes. Xphos Pd G2 (457 mg, 580.83 μmol) was added and again degassed for 5 minutes. Trimethylboroxine (50% solution in THF) (24.605 mL of 50% w/v, 0.0980 mol) was added and heated to 80° C. for 16 hours. Reaction mixture was diluted with water (200 ml), and extracted with Ethyl acetate (3×150 ml). Separated organic layer was dried over Na₂SO₄, filtered, and concentrated under reduced pressure. Purification by column chromatography (Eluent: 20% EtOAc in pet ether) afforded the product S1072-(5-methyl-3-thienyl)ethanol (950 mg, 66%)¹H NMR (400 MHz, DMSO-d₆) δ 6.87 (d, J=0.8 Hz, 1H), 6.68 (s, 1H), 4.59 (t, J=5.2 Hz, 1H), 3.58-3.53 (m, 2H), 2.63 (t, J=7.2 Hz, 2H), 2.38 (d, J=0.8 Hz, 3H) as yellow liquid.

### Preparation S108

### 2-(5-methyl-2-thienyl)ethanol (S108)

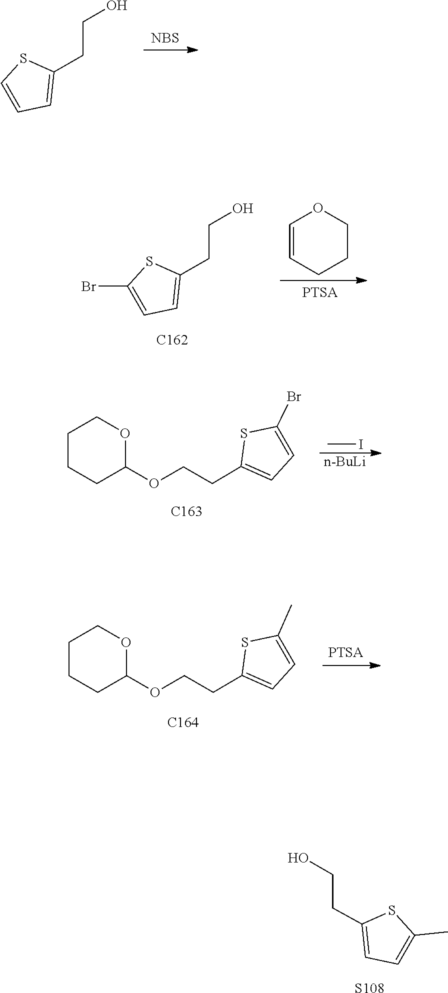

### Step 1: 2-(5-bromo-2-thienyl)ethanol (C162)

A solution of 2-(2-thienyl)ethanol (15 g, 0.1170 mol) in DMF (150.00 mL) was drop-wise added to a solution of NBS (20.824 g, 0.1170 mol) in DMF at −10° C. Reaction was stirred at room temperature for 16 hours. Reaction mixture was quenched with water (300 mL) and extracted with Ethyl acetate (200 mL×3). Organic layer was washed with 6% KOH solution, ice water (150 mL×2) and brine (150 mL). Organic layer was dried over sodium sulfate and concentrated. Purification by column chromatography (Eluent: 10% EtOAc in pet ether) afforded the product 2-(5-bromo-2-thienyl)ethanol (C162) (20.5 g, 79%)2-(5-bromo-2-thienyl)ethanol (20.5 g, 79%) ¹H NMR (400 MHz, Chloroform-d): δ 6.89 (d, J=3.6 Hz, 1H), 6.64-6.28 (m, 1H), 3.82 (t, J=6.0 Hz, 2H), 2.99 (t, J=6.0 Hz, 2H).

### Step 2: 2-[2-(5-bromo-2-thienyl)ethoxy]tetrahydropyran (C163)

To a stirred solution of 2-(5-bromo-2-thienyl)ethanol (C162) (20 g, 0.0869 mol) and 3,4-dihydro-2H-pyran (10.969 g, 0.1304 mol) in THF (80.000 mL) was added with PTSA (603 mg, 0.5636 mL, 0.0035 mol) and reaction was stirred for 24 hour at room temperature. The reaction mixture was diluted with ethyl acetate, washed with sat. sodium bicarbonate solution (50 mL), water, and brine. The organic layer was separated, dried over sodium sulfate, and concentrated. Purification by silica gel chromatography (Gradient: 0-5% EtOAc in Pet Ether) yielded the product 2-[2-(5-bromo-2-thienyl)ethoxy]tetrahydropyran (C163) (18.5 g, 64%) ¹H NMR (400 MHz, Chloroform-d): δ 6.86 (d, J=3.6 Hz, 1H), 6.61-6.60 (m, 1H), 4.62 (t, J=3.6 Hz, 1H), 3.99-3.50 (m, 4H), 3.05-3.01 (m, 2H), 1.73-1.50 (m, 6H).

### Step 3: 2-[2-(5-methyl-2-thienyl)ethoxy]tetrahydropyran (C164)

To a solution of 2-[2-(5-bromo-2-thienyl)ethoxy]tetrahydropyran (C163) (19 g, 0.0555 mol) in THF (380.00 mL) was added n-BuLi (33.320 mL of 2.5 M, 0.0833 mol) drop-wise at −78° C. Reaction was stirred for one hour at −78° C. Iodomethane (15.755 g, 6.9101 mL, 0.1110 mol) was added drop-wise at −78° C. and the reaction mixture was allowed to stir at room temperature for 16 hours. The reaction mixture was quenched with saturated NH₄Cl solution and diluted with water. The aqueous layer was extracted with ethyl acetate (2×250 mL). Purification by silica gel chromatography (Eluent: 100% Pet Ether) yielded the product 2-[2-(5-methyl-2-thienyl)ethoxy]tetrahydropyran (C₃₂) (19 g, 130%) ¹H NMR (400 MHz, Chloroform-d): δ 6.61 (d, J=3.2 Hz, 1H), 6.55-6.54 (m, 1H), 4.63 (m, 1H), 3.96-3.50 (m, 4H), 3.03 (t, J=2.8 Hz, 2H), 2.42 (s, 3H), 1.72-1.42 (m, 6H).

### Step 4: 2-(5-methyl-2-thienyl)ethanol (S108)

To a solution of 2-[2-(5-methyl-2-thienyl)ethoxy]tetrahydropyran (14 g, 0.0532 mol) in MeOH (280.00 mL) was added with PTSA (10.9 g, 10.187 mL, 0.0633 mol) at room temperature Reaction was stirred for 24 hours. Reaction mixture was diluted with ethyl acetate (500 mL) and then washed with water (200 mL). The organic layer was washed with saturated aqueous sodium bicarbonate (100 mL×2) solution. Aqueous layer was again extracted with ethyl acetate (100 ml×2). Combined organic layer was dried over sodium sulfate. Purification by silica gel chromatography (Gradient: 0-15% EtOAc in Pet Ether) yielded the product 2-(5-methyl-2-thienyl)ethanol (6.56 g, 82%) ¹H NMR (400 MHz, DMSO-d₆): δ 6.61 (d, J=3.6 Hz, 1H), 6.58-6.57 (d, J=4.0 Hz, 1H), 4.73 (t, J=5.2 Hz, 1H), 3.58-3.53 (m, 2H), 2.82 (t, J=6.8 Hz, 2H), 2.36 (s, 3H).

### Preparation S109

### [4-(2-hydroxyethyl)-2-(trifluoromethyl)-3-thienyl]methyl acetate (S109)

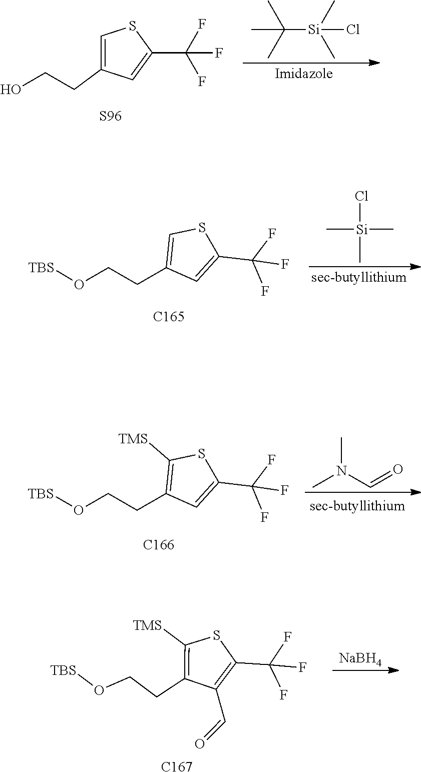

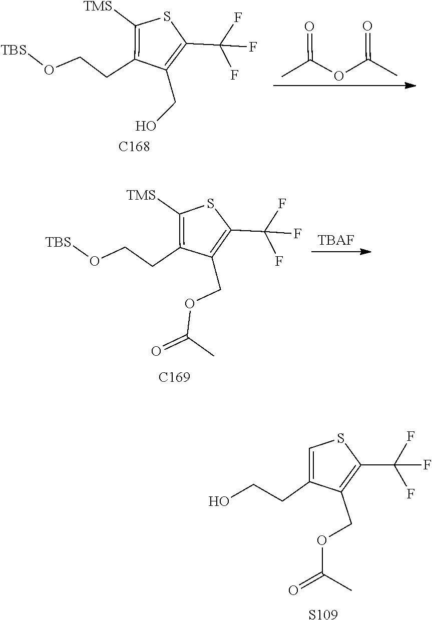

### Step 1: tert-butyl-dimethyl-[2-[5-(trifluoromethyl)-3-thienyl]ethoxy]silane (C165)

To a mixture of 2-[5-(trifluoromethyl)-3-thienyl]ethanol (S96) (500 mg, 2.498 mmol) in DCM (10 mL) was added imidazole (190 mg, 2.791 mmol) followed by TBSCl (420 mg, 2.787 mmol) which immediately precipitated a white solid. The solid was filtered and the organic layer was washed with 1 N HCl (10 mL), brine (10 mL), dried with magnesium sulfate, filtered, and concentrated. Purification by silica gel chromatography (Gradient: 0-30% EtOAc in heptane) yielded the product. The product-containing fractions were pooled and concentrated. Yield was assumed to be quantitative and crude was telescoped to the next step.

### Step 2: tert-butyl-dimethyl-[2-[5-(trifluoromethyl)-2-trimethylsilyl-3-thienyl]ethoxy]silane (C166)

The mixture from step 1 in THF (10 mL) was cooled to −78° C. and sec-butyllithium (2.3 mL of 1.4 M, 3.220 mmol) was added followed by TMSCl (3 mL of 1 M, 3.000 mmol). After 5 minutes, the yellow mixture was quenched with sat. aq. ammonium chloride. The mixture was diluted with water (10 mL) and TBME (10 mL). The organic layer was washed with brine, dried with magnesium sulfate, filtered and concentrated. Purification by silica gel chromatography (Gradient: 0-10% EtOAc in heptane) yielded the product, tert-butyl-dimethyl-[2-[5-(trifluoromethyl)-2-trimethylsilyl-3-thienyl]ethoxy]silane (C166) (400 mg, 42%)¹H NMR (300 MHz, Chloroform-d) δ 7.41 (d, J=1.2 Hz, 1H), 3.80-3.75 (m, 2H), 2.87 (t, J=6.8 Hz, 2H), 0.87 (s, 9H), 0.36 (s, 9H), −0.00 (d, J=2.2 Hz, 6H).

### Step 3: 4-[2-[tert-butyl(dimethyl)silyl]oxyethyl]-2-(trifluoromethyl)-5-trimethylsilyl-thiophene-3-carbaldehyde (C167)

To a mixture of tert-butyl-dimethyl-[2-[5-(trifluoromethyl)-2-trimethylsilyl-3-thienyl]ethoxy]silane C166 (400 mg, 1.024 mmol) in THF (10 mL) cooled to −78° C. was added sec-butyllithium (1.2 mL of 1.4 M, 1.680 mmol) followed by N,N-dimethylformamide (3 mL of 1 M, 3.000 mmol). After 5 minutes, the yellow mixture was quenched with sat. aq. ammonium chloride. The mixture was diluted with ethyl acetate (20 mL) and water (20 mL) and separated. The organic layer was washed with brine (20 mL), dried with magnesium sulfate, filtered and concentrated. Purification by silica gel chromatography (Eluent: 100% heptane) yielded the product. The mixture was concentrated, diluted with heptane (5 mL) and washed with water (5 mL). The organic layer was passed over a phase separator, concentrated, and telescoped directly to the next step.

### Step 4: [4-[2-[tert-butyl(dimethyl)silyl]oxyethyl]-2-(trifluoromethyl)-5-trimethylsilyl-3-thienyl]methanol (C168)

(C167) was diluted in MeOH (1 mL) and to the mixture was added NaBH₄ (7 mg, 0.1850 mmol). After 10 minutes the mixture was concentrated, and rediluted in heptane (2 mL) and water (2 mL). The organic layer was removed and the aqueous layer was extracted with additional heptane. The organic layer was passed over a phase separator and concentrated. Purification by silica gel chromatography (Gradient: 0-10% EtOAc in heptane) yielded the product (C168). ¹H NMR (300 MHz, Chloroform-d) δ 4.65 (d, J=6.3 Hz, 2H), 4.00-3.72 (m, 2H), 3.34 (t, J=6.3 Hz, 1H), 2.97 (t, J=6.1 Hz, 2H), 0.82 (s, 10H), 0.36 (s, 9H).

### Step 5: [4-[2-[tert-butyl(dimethyl)silyl]oxyethyl]-2-(trifluoromethyl)-5-trimethylsilyl-3-thienyl]methyl acetate (C₁₆₉)

To (C168) in DCM (4 mL) was added DMAP (2 mg, 0.01637 mmol) and DIPEA (50 μL, 0.2871 mmol) followed by Ac20 (30 μL, 0.3180 mmol). The mixture was concentrated, diluted with heptane (5 mL) and washed with water (5 mL). The organic layer was passed over a phase separator and concentrated to yield the product.

### Step 6: [4-(2-hydroxyethyl)-2-(trifluoromethyl)-3-thienyl]methyl acetate (S109)

(C169) from step 5 was diluted with EtOAc (2 mL) and to the mixture was added a THF solution of TBAF (1 mL of 1 M, 1.000 mmol) and the mixture was stirred. After 5 minutes, full deprotection of the TBS group was observed. The reaction was stirred for 48 hours. The mixture was diluted with additional EtOAc (3 mL), washed with water, passed over a phase separator and concentrated. Purification by silica gel chromatography (Gradient: 0-60% EtOAc in heptane) yielded the product [4-(2-hydroxyethyl)-2-(trifluoromethyl)-3-thienyl]methyl acetate (35 mg, 12%) ¹H NMR (300 MHz, Chloroform-d) δ 7.26 (s, 1H), 5.14 (d, J=1.1 Hz, 2H), 3.86 (t, J=6.4 Hz, 2H), 2.97-2.74 (m, 2H), 2.07 (s, 3H), 1.80 (s, 1H). LCMS m/z 269.21 [M+H]⁺.

### Compound 1043

### 5-((2′-ethyl-6′,7′-dihydrospiro[piperidine-4,4′-thieno[3,2-c]pyran]-1-yl)methyl)-N-((1r,4r)-4-hydroxycyclohexyl)picolinamide (1043)

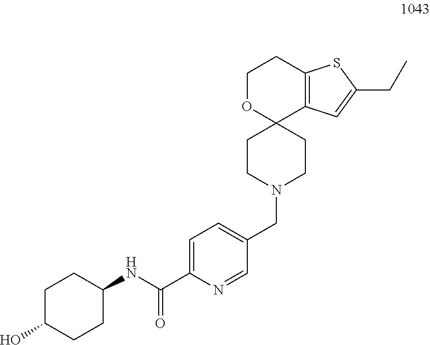

### Step 1: 2-ethylspiro[6,7-dihydrothieno[3,2-c]pyran-4,4′-piperidine](S110)

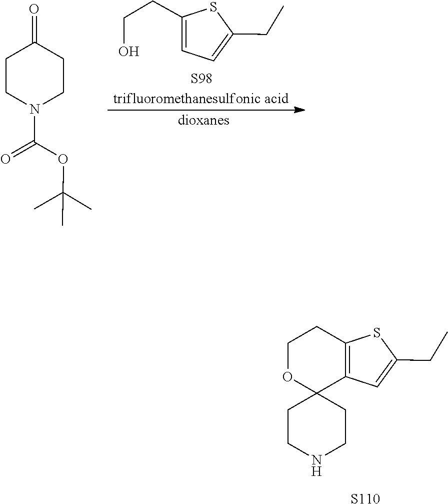

To a stirred solution of 2-(5-ethyl-2-thienyl)ethanol (S98) (3 g, 18.22 mmol) and tert-butyl 4-oxopiperidine-1-carboxylate (3.3 g, 16.56 mmol) in 1,4-dioxane (50 mL) at 0° C. was added trifluoromethanesulfonic acid (4.5 mL, 50.85 mmol). The reaction mixture was slowly warmed to room temperature with stirring. The reaction diluted with water, then adjusted to pH 8 with 2M Na₂CO₃ and extracted with ethyl acetate. The combined organics were dried over anhydrous sodium sulfate, filtered, and concentrated in vacuo. Reverse phase purification with TFA modifier, followed by lyophilization overnight provided the amorphous white solid 2-ethylspiro[6,7-dihydrothieno[3,2-c]pyran-4,4′-piperidine](S110, trifluoroacetate salt) (5.21 g, 29%) ESI-MS m/z calc. 237.11873, found 237.82 (M+1)+.

### Step 2: methyl 5-[(2-ethylspiro[6,7-dihydrothieno[3,2-c]pyran-4,4′-piperidine]-1′-yl)methyl]pyridine-2-carboxylate (S111)

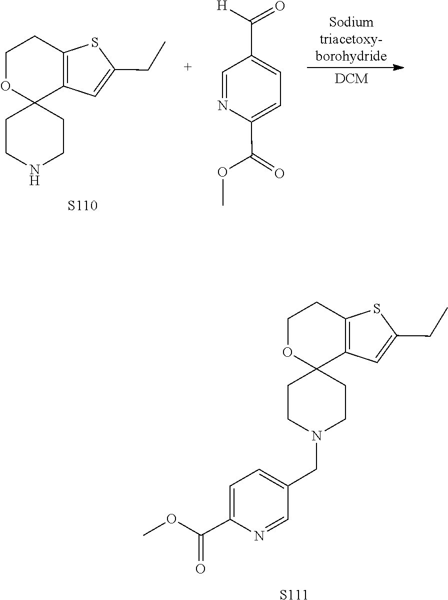 [SMILES: "S110" ; CCc1cc2c(s1)CCOC21CCNCC1 ; COC(=O)c1ccc(C=O)cn1 ; CCc1cc2c(s1)CCOC21CCN(Cc2ccc(C(=O)OC)nc2)CC1 ; "S111"]

A solution of 2-ethylspiro[6,7-dihydrothieno[3,2-c]pyran-4,4′-piperidine](3.3 g, 13.90 mmol) in dichloromethane (95 mL) was treated with methyl 5-formylpyridine-2-carboxylate (S110) (3.5 g, 21.19 mmol) and sodium triacetoxyborohydride (5.9 g, 27.84 mmol). The resulting solution was stirred at room temperature overnight. The reaction was quenched by partitioning between saturated NaHCO₃, the organics were dried over anhydrous sodium sulfate, filtered, and concentrated in vacuo. Purification by HPLC: 10-90% ACN in Water (TFA modifier) C18 column, then lyophilization overnight afforded methyl 5-[(2-ethylspiro[6,7-dihydrothieno[3,2-c]pyran-4,4′-piperidine]-1′-yl)methyl]pyridine-2-carboxylate (Trifluoroacetate salt) (2.3207 g, 32%)¹H NMR (300 MHz, Chloroform-d) δ 8.77 (dd, J=2.1, 1.0 Hz, 1H), 8.29-8.18 (m, 2H), 6.45 (d, J=1.0 Hz, 1H), 4.33 (s, 2H), 4.05 (s, 3H), 3.90 (t, J=5.3 Hz, 2H), 3.41 (d, J=11.5 Hz, 2H), 3.19 (t, J=12.1 Hz, 2H), 2.82-2.71 (m, 4H), 2.38 (td, J=14.2, 13.7, 4.3 Hz, 2H), 2.05-1.96 (m, 2H), 1.27 (t, J=7.5 Hz, 3H). ESI-MS m/z calc. 386.1664, found 387.47 (M+1)⁺.

### Step 3: Synthesis of 5-[(2-ethylspiro[6,7-dihydrothieno[3,2-c]pyran-4,4′-piperidine]-1′-yl)methyl]pyridine-2-carboxylic acid (S112)

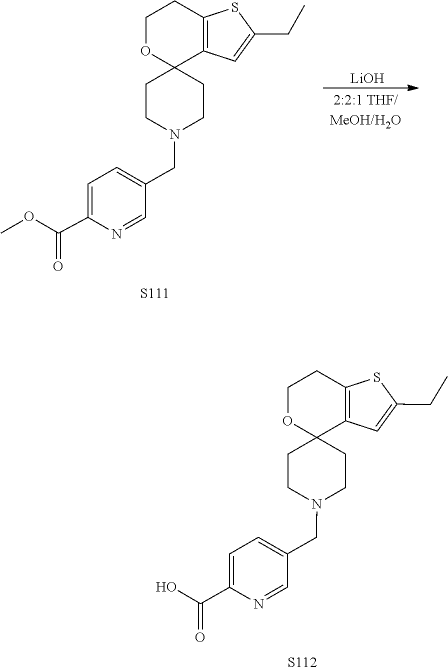 [SMILES: CCc1cc2c(s1)CCOC21CCN(Cc2ccc(C(=O)OC)nc2)CC1 ; "LiOH" ; "S111" ; CCc1cc2c(s1)CCOC21CCN(Cc2ccc(C(=O)O)nc2)CC1 ; "S112"]

A solution of methyl 5-[(2-ethylspiro[6,7-dihydrothieno[3,2-c]pyran-4,4′-piperidine]-1′-yl)methyl]pyridine-2-carboxylate (Trifluoroacetate salt) (2.3091 g, 4.532 mmol) in THF (9 mL)/MeOH (9 mL)/Water (4.5 mL) was treated with LiOH (220 mg, 9.187 mmol) and allowed to stir at ambient temperature for approximately 3 hours. The reaction was concentrated in vacuo to remove the organics. The remaining aqueous was acidified to pH 3 with 1N HCl and partitioned with ethyl acetate. The combined organics were dried over anhydrous sodium sulfate, filtered, and concentrated in vacuo. Lyophilization from acetonitrile/water overnight to afford 5-[(2-ethylspiro[6,7-dihydrothieno[3,2-c]pyran-4,4′-piperidine]-1′-yl)methyl]pyridine-2-carboxylic acid (467.9 mg, 26%) ¹H NMR (300 MHz, Chloroform-d) δ 8.81 (s, 1H), 8.27 (d, J=11.9 Hz, 2H), 6.43 (s, 1H), 4.37 (s, 2H), 3.92 (t, J=5.2 Hz, 2H), 3.53 (s, 2H), 3.26 (s, 2H), 2.85-2.70 (m, 4H), 2.35 (s, 2H), 2.05 (d, J=13.8 Hz, 2H), 1.27 (t, J=7.5 Hz, 3H). ESI-MS m/z calc. 372.15076, found 373.48 (M+1)⁺.

### Step 4: Synthesis of 5-((2′-ethyl-6′,7′-dihydrospiro[piperidine-4,4′-thieno[3,2-c]pyran]-1-yl)methyl)-N-((1r,4r)-4-hydroxycyclohexyl)picolinamide (1043)

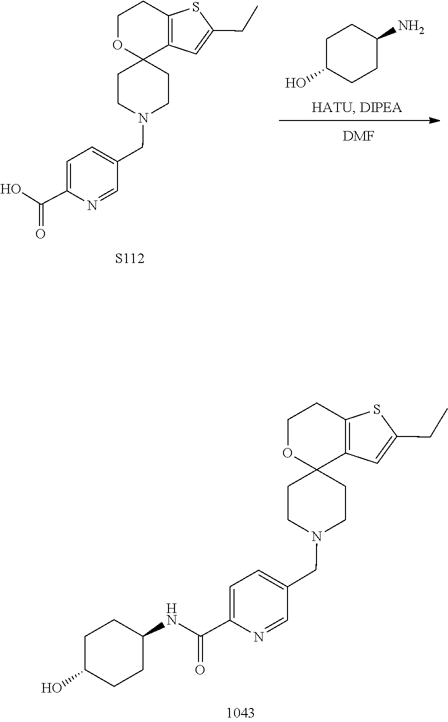

A solution of 5-[(2-ethylspiro[6,7-dihydrothieno[3,2-c]pyran-4,4′-piperidine]-1′-yl)methyl]pyridine-2-carboxylic acid (15 mg, 0.038 mmol), HATU (22 mg, 0.057 mmol), and DIPEA (12 mg, 0.095 mmol) in DMF (1 mL) was added to a vial containing trans-4-aminocyclohexanol (8.8 mg, 0.076 mmol). The resulting solution was stirred at ambient temperature for 16 hours, were filtered, and directly purified by HPLC with TFA modifier to provide 5-((2′-ethyl-6′,7′-dihydrospiro[piperidine-4,4′-thieno[3,2-c]pyran]-1-yl)methyl)-N-((1r,4r)-4-hydroxycyclohexyl)picolinamide (5 mg, 28%). ESI-MS m/z calc. 470.24, found 470.247 (M+1)⁺.

Compounds 1044-1065 (see Table 49) were prepared from S112 using the appropriate amines employing the same method as described for compound 1043.

TABLE 49
Structure and physicochemical data for compounds 1025-1026

| | | | ¹H NMR; LCMS |
|---|---|---|---|
| Cmpd | Product | Amine | m/z [M + H]⁺ |
| 1044 | 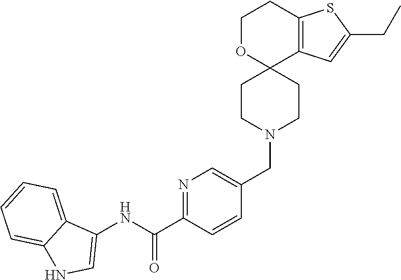 [SMILES: CCc1cc2c(s1)CCOC21CCN(Cc2ccc(C(=O)Nc3c[nH]c4ccccc34)nc2)CC1] | 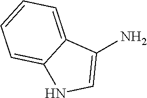 | ESI-MS m/z calc. 486.628, found 487.217 (M + 1)+ |
| 1045 | 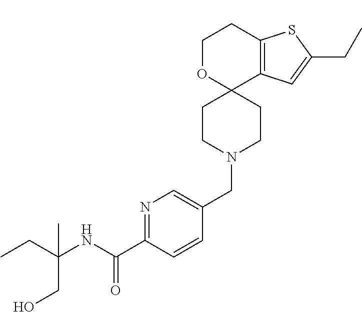 | 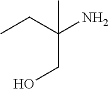 | ESI-MS m/z calc. 457.629, found 458.248 (M + 1)+ |
| 1046 | 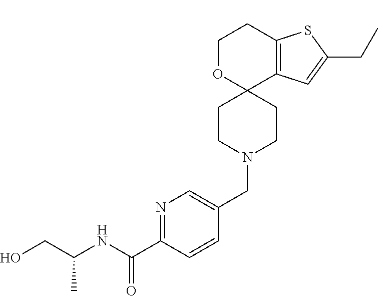 | 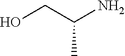 | ESI-MS m/z calc. 429.576, found 430.215 (M + 1)+ |
| 1047 | 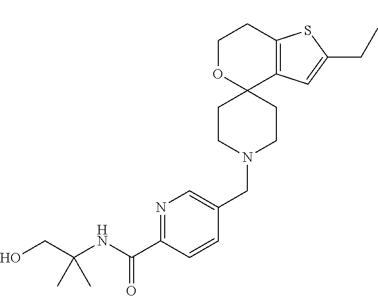 | 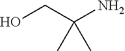 | ESI-MS m/z calc. 443.602, found 444.232 (M + 1)+ |
| 1048 | 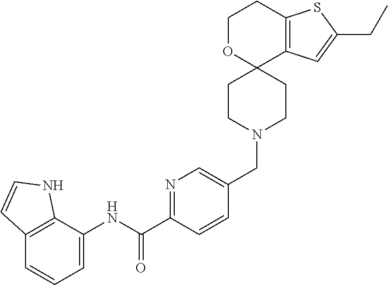 [SMILES: CCc1cc2c(s1)CCOC21CCN(Cc2ccc(C(=O)Nc3cccc4cc[nH]c34)nc2)CC1] | 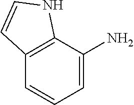 | ESI-MS m/z calc. 486.628, found 487.217 (M + 1)+ |
| 1049 | 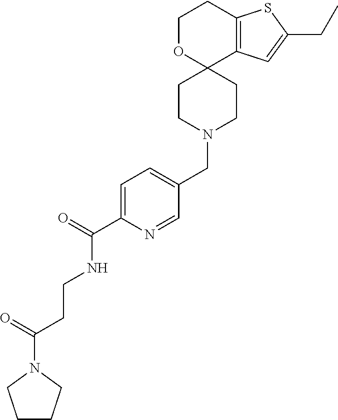 [SMILES: CCc1cc2c(s1)CCOC21CCN(Cc2ccc(C(=O)NCCC(=O)N3CCCC3)nc2)CC1] | 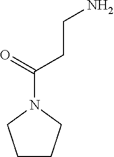 | ESI-MS m/z calc. 496.665, found 497.258 (M + 1)+ |
| 1050 | 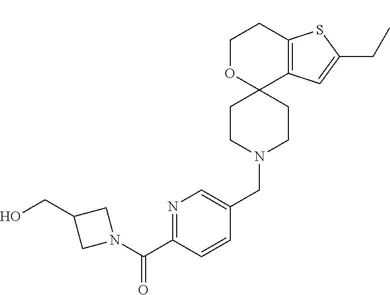 | 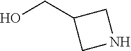 | ESI-MS m/z calc. 441.586, found 442.216 (M + 1)+ |
| 1051 | 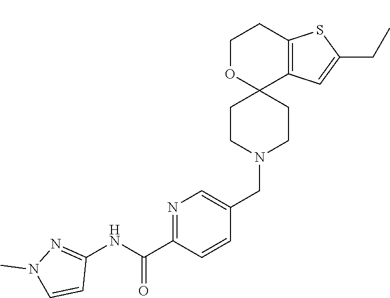 | 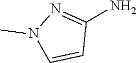 | ESI-MS m/z calc. 451.584, found 452.212 (M + 1)+ |
| 1052 | 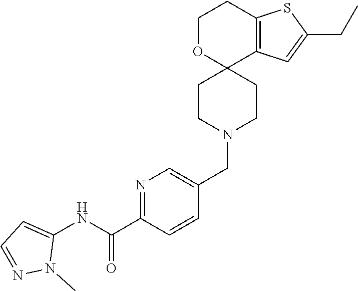 [SMILES: CCc1cc2c(s1)CCOC21CCN(Cc2ccc(C(=O)Nc3ccnn3C)nc2)CC1] | 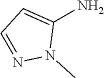 | ESI-MS m/z calc. 451.584, found 452.212 (M + 1)+ |
| 1053 | 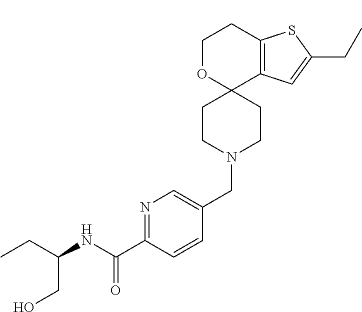 | 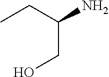 | ESI-MS m/z calc. 443.602, found 444.232 (M + 1)+ |
| 1054 | 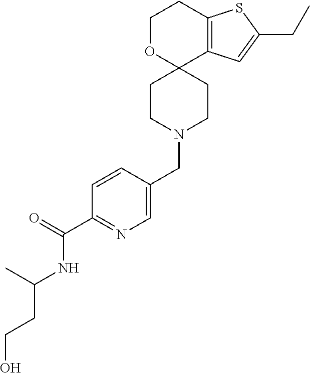 [SMILES: CCc1cc2c(s1)CCOC21CCN(Cc2ccc(C(=O)NC(C)CCO)nc2)CC1] | 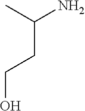 | ESI-MS m/z calc. 443.602, found 444.232 (M + 1)+ |
| 1055 | 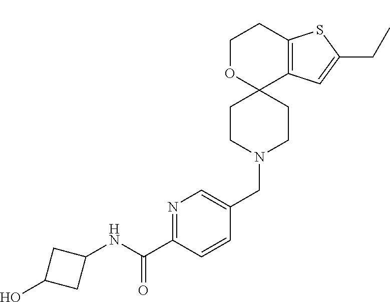 | 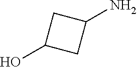 | ESI-MS m/z calc. 441.586, found 442.216 (M + 1)+ |
| 1056 | 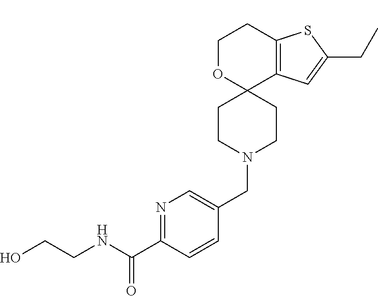 | 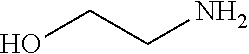 | ESI-MS m/z calc. 415.549, found 416.201 (M + 1)+ |
| 1057 | 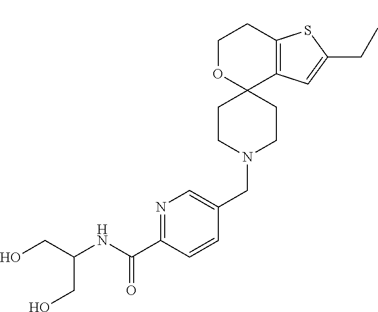 | 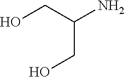 | ESI-MS m/z calc. 445.575, found 446.211 (M + 1)+ |
| 1058 | 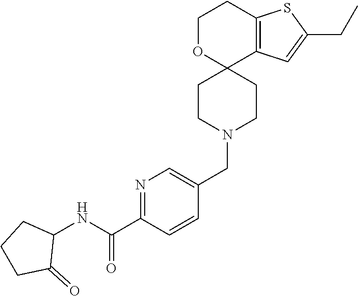 [SMILES: CCc1cc2c(s1)CCOC21CCN(Cc2ccc(C(=O)NC3CCCC3=O)nc2)CC1] | 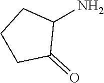 | ESI-MS m/z calc. 453.597, found 454.216 (M + 1)+ |
| 1059 | 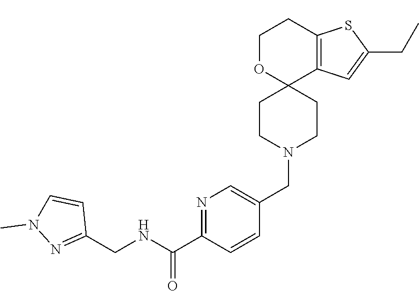 | 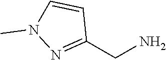 | ESI-MS m/z calc. 465.611, found 466.227 (M + 1)+ |
| 1060 | 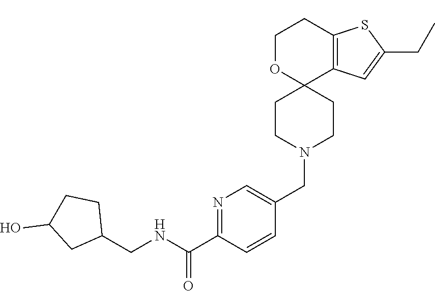 | 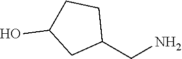 | ESI-MS m/z calc. 469.639, found 470.248 (M + 1)+ |
| 1061 | 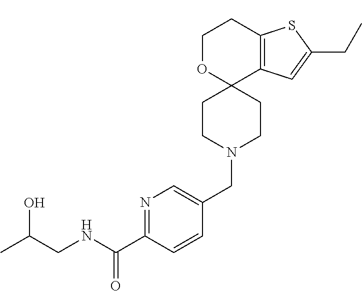 | 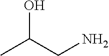 | ESI-MS m/z calc. 429.576, found 430.216 (M + 1)+ |
| 1062 | 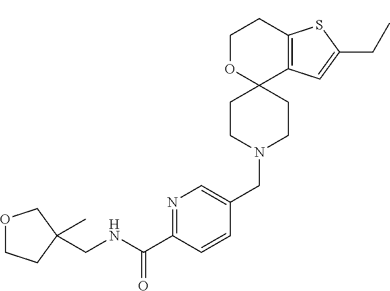 | 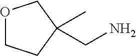 | ESI-MS m/z calc. 469.639, found 470.248 (M + 1)+ |
| 1063 | 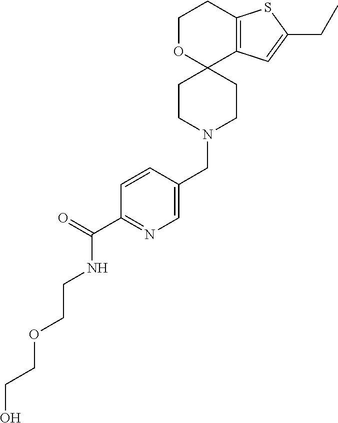 [SMILES: CCc1cc2c(s1)CCOC21CCN(Cc2ccc(C(=O)NCCOCCO)nc2)CC1] | 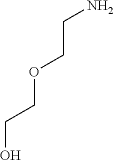 | ESI-MS m/z calc. 459.602, found 460.227 (M + 1)+ |
| 1064 | 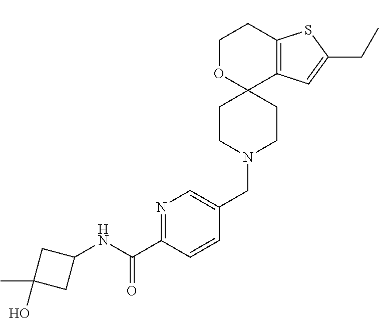 | 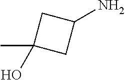 | ESI-MS m/z calc. 455.613, found 456.232 (M + 1)+ |
| 1065 | 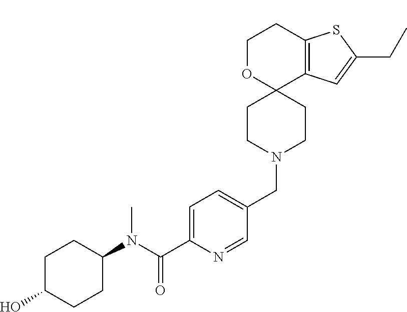 | 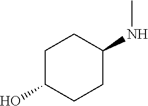 | ESI-MS m/z calc. 483.666, found 484.263 (M + 1)+ |

### Compound 1066

### (3R,4R)-1-(5-((2′-ethyl-6′,7′-dihydrospiro[piperidine-4,4′-thieno[3,2-c]pyran]-1-yl)methyl)pyridin-2-yl)pyrrolidine-3,4-diol (1066)

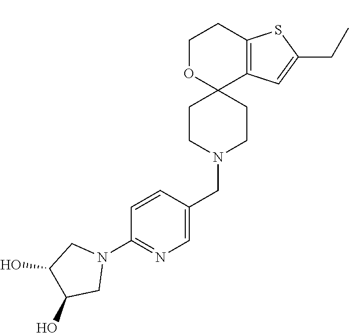

### Step 1: Synthesis of 1′-[(6-bromo-3-pyridyl)methyl]-2-ethyl-spiro[6,7-dihydrothieno[3,2-c]pyran-4,4′-piperidine](S113)

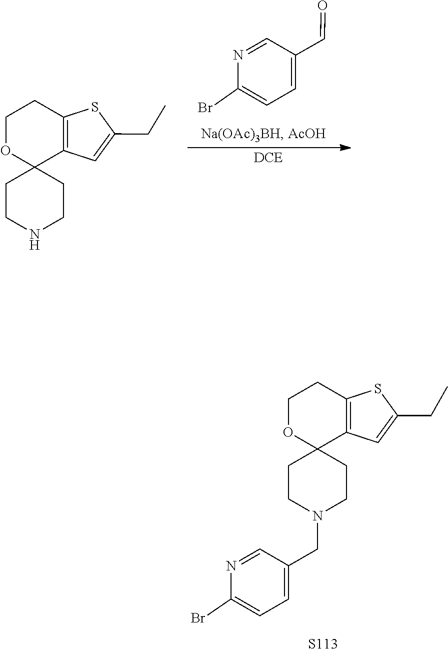

A solution of 2-ethylspiro[6,7-dihydrothieno[3,2-c]pyran-4,4′-piperidine](2.5 g, 10.53 mmol), 6-bromopyridine-3-carbaldehyde (1.9 g, 10.21 mmol), and AcOH (640 μL, 11.25 mmol) in dichloroethane (45 mL) was stirred at room temperature for 10 minutes, followed by addition of Na(OAc)₃BH (6.6 g, 31.14 mmol). The resulting mixture was stirred at room temperature overnight then concentrated in vacuo. Purification by flash column chromatography (0-10% MeOH in DCM) provided 1′-[(6-bromo-3-pyridyl)methyl]-2-ethyl-spiro[6,7-dihydrothieno[3,2-c]pyran-4,4′-piperidine](2.06 g, 48%) ¹H NMR (300 MHz, Chloroform-d) δ 8.34-8.28 (m, 1H), 7.58 (dd, J=8.1, 2.5 Hz, 1H), 7.44 (dd, J=8.1, 0.7 Hz, 1H), 6.47 (d, J=1.1 Hz, 1H), 3.90 (t, J=5.4 Hz, 2H), 3.50 (s, 2H), 2.80-2.71 (m, 4H), 2.70-2.60 (m, 2H), 2.40 (td, J=11.5, 3.3 Hz, 2H), 1.97-1.81 (m, 4H), 1.27 (t, J=7.5 Hz, 3H). ESI-MS m/z calc. 406.07144, found 407.1 (M+1)⁺.

### Step 2: Synthesis of (3R,4R)-1-(5-((2′-ethyl-6′,7′-dihydrospiro[piperidine-4,4′-thieno[3,2-c]pyran]-1-yl)methyl)pyridin-2-yl)pyrrolidine-3,4-diol (1066)

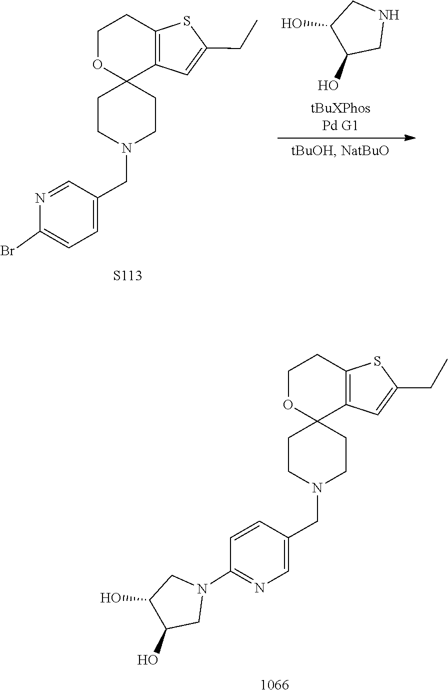

To a vial containing 1′-[(6-bromo-3-pyridyl)methyl]-2-ethyl-spiro[6,7-dihydrothieno[3,2-c]pyran-4,4′-piperidine](15 mg, 0.04 mmol), (3R,4R)-pyrrolidine-3,4-diol (4.4 mg, 0.042 mmol), tBuXPhos Pd G1(1.2 mg, 0.0018 mmol), and tBuOH (0.5 mL) was treated with 2-methylpropan-2-olate (Sodium salt) (39 uL, 2M), sealed under nitrogen and stirred for 3 hours at 80° C. The reaction was quenched by addition of methanol and purified by reverse phase HPLC with TFA modifier to afford (3R,4R)-1-(5-((2′-ethyl-6′,7′-dihydrospiro[piperidine-4,4′-thieno[3,2-c]pyran]-1-yl)methyl)pyridin-2-yl)pyrrolidine-3,4-diol (6 mg, 28). ESI-MS m/z calc. 429.576, found 430.216 (M+1)⁺.

### Compounds 1067-1072

Compounds 1067-1072 (see Table 50) were prepared from S113 using the appropriate amines employing the same method as described for compound 1066.

TABLE 50
Structure and physicochemical data for compounds 1067-1072

| | | | ¹H NMR; LCMS |
|---|---|---|---|
| Cmpd | Product | Amine | m/z [M + H]⁺ |
| 1067 | 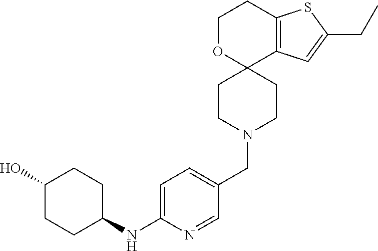 [SMILES: CCc1cc2c(s1)CCOC21CCN(Cc2ccc(N[C@H]3CC[C@H](O)CC3)nc2)CC1] | 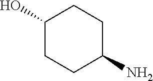 | ESI-MS m/z calc. 441.629, found 442.253 (M + 1)+ |
| 1068 | 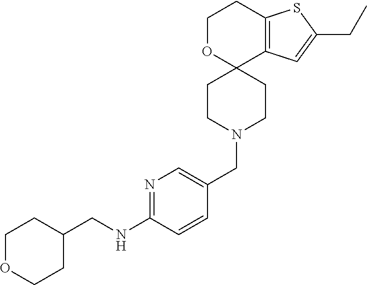 [SMILES: CCc1cc2c(s1)CCOC21CCN(Cc2ccc(NCC3CCOCC3)nc2)CC1] | 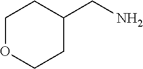 | ESI-MS m/z calc. 441.629, found 442.253 (M + 1)+ |
| 1069 | 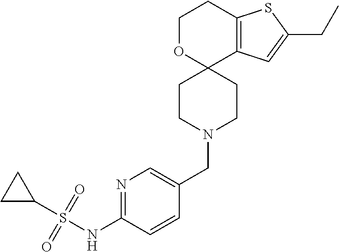 [SMILES: CCc1cc2c(s1)CCOC21CCN(Cc2ccc(NS(=O)(=O)C3CC3)nc2)CC1] | 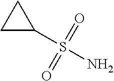 | ESI-MS m/z calc. 447.614, found 448.172 (M + 1)+ |
| 1070 | 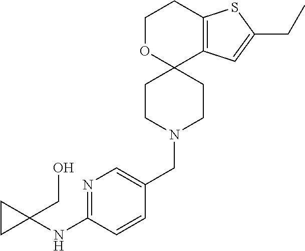 [SMILES: CCc1cc2c(s1)CCOC21CCN(Cc2ccc(NC3(CO)CC3)nc2)CC1] | 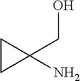 | ESI-MS m/z calc. 413.576, found 414.221 (M + 1)+ |
| 1071 | 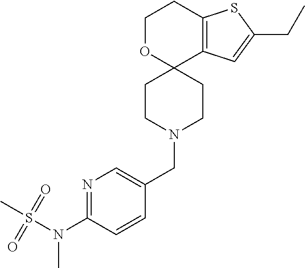 [SMILES: CCc1cc2c(s1)CCOC21CCN(Cc2ccc(N(C)S(C)(=O)=O)nc2)CC1] | 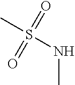 | ESI-MS m/z calc. 435.603, found 436.172 (M + 1)+ |
| 1072 | 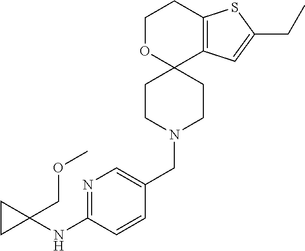 [SMILES: CCc1cc2c(s1)CCOC21CCN(Cc2ccc(NC3(COC)CC3)nc2)CC1] | 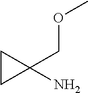 | ESI-MS m/z calc. 427.603, found 428.237 (M + 1)+ |

### Compound 1073

### (1r,4r)-4-((5-((2′-ethyl-6′,7′-dihydrospiro[piperidine-4,4′-thieno[3,2-c]pyran]-1-yl)methyl)pyrimidin-2-yl)amino)cyclohexan-1-ol (1073)

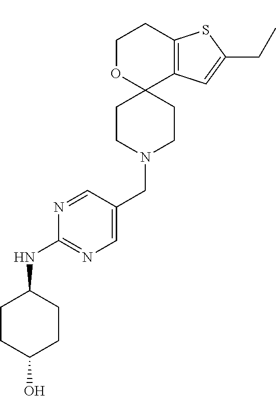

### Step 1: Synthesis of 1′-[(2-chloropyrimidin-5-yl)methyl]-2-ethyl-spiro[6,7-dihydrothieno[3,2-c]pyran-4,4′-piperidine](S114)

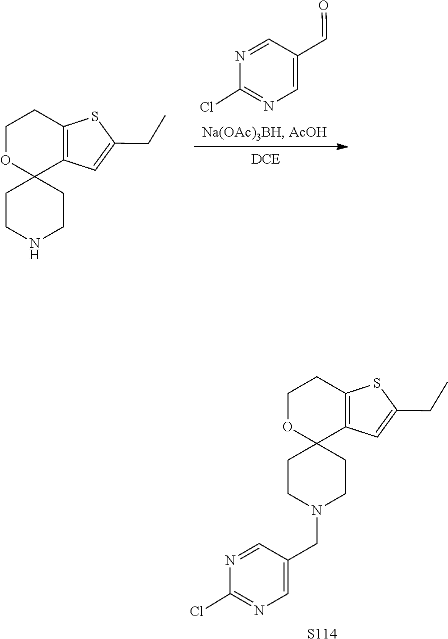 [SMILES: O=Cc1cnc(Cl)nc1 ; CCc1cc2c(s1)CCOC21CCNCC1 ; CCc1cc2c(s1)CCOC21CCN(Cc2cnc(Cl)nc2)CC1 ; "S114"]

In a reaction vial was added 2-ethylspiro[6,7-dihydrothieno[3,2-c]pyran-4,4′-piperidine] (2.5 g, 10.53 mmol), 2-chloropyrimidine-5-carbaldehyde (1.7 g, 11.93 mmol), and AcOH (600 μL, 10.55 mmol) in DCE (42 mL). triacetoxyboranuide (Sodium salt) (6.7 g, 31.61 mmol) added, and reaction stirred at ambient temperature overnight. Reaction was then quenched with water, extracted three times with DCM, and concentrated in vacuo. The crude residue was purified by reverse phase HPLC (HCl modifier) to provide 1′-[(2-chloropyrimidin-5-yl)methyl]-2-ethyl-spiro[6,7-dihydrothieno[3,2-c]pyran-4,4′-piperidine](1.2 g, 29%) ESI-MS m/z calc. 363.11722, found 363.73 (M+1)⁺.

### Step 2: Synthesis of (1r,4r)-4-((5-((2′-ethyl-6′,7′-dihydrospiro[piperidine-4,4′-thieno[3,2-c]pyran]-1-yl)methyl)pyrimidin-2-yl)amino)cyclohexan-1-ol (1073)

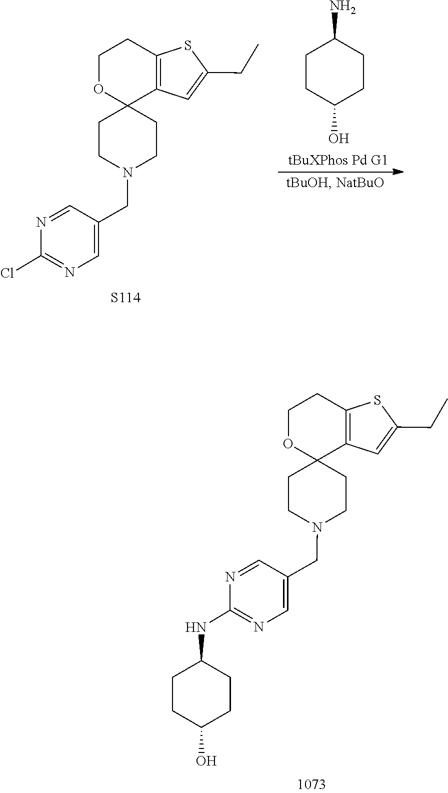 [SMILES: CCc1cc2c(s1)CCOC21CCN(Cc2cnc(Cl)nc2)CC1 ; N[C@H]1CC[C@H](O)CC1 ; "S114" ; CCc1cc2c(s1)CCOC21CCN(Cc2cnc(N[C@H]3CC[C@H](O)CC3)nc2)CC1]

To a vial containing 1′-[(2-chloropyrimidin-5-yl)methyl]-2-ethyl-spiro[6,7-dihydrothieno[3,2-c]pyran-4,4′-piperidine](15 mg, 0.04 mmol), trans-4-aminocyclohexanol (5.5 mg, 0.047 mmol), tBuXPhos Pd G1(1.2 mg, 0.002 mmol), and tBuOH (0.5 mL) was treated with 2-methylpropan-2-olate (Sodium salt) (39 uL, 2M), sealed under nitrogen and stirred for 3 hours at 80° C. The reaction was quenched by addition of methanol and purified by reverse phase HPLC with TFA modifier to afford (1r,4r)-4-((5-((2′-ethyl-6′,7′-dihydrospiro[piperidine-4,4′-thieno[3,2-c]pyran]-1-yl)methyl)pyrimidin-2-yl)amino)cyclohexan-1-ol (15 mg, 670%) as the TFA salt. ESI-MS m/z calc. 442.24, found 443.24 (M+1)⁺.

### Compounds 1074-1078

Compounds 1074-1078 (see Table 51) were prepared from S114 using the appropriate amines employing the same method as described for compound 1073.

TABLE 51
Structure and physicochemical data for compounds 1074-1078

| | | | ¹H NMR; LCMS |
|---|---|---|---|
| Compound | Product | Amines | m/z [M + H]⁺ |
| 1074 | 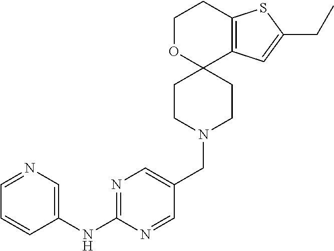 [SMILES: CCc1cc2c(s1)CCOC21CCN(Cc2cnc(Nc3cccnc3)nc2)CC1] | 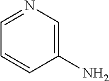 | ESI-MS m/z calc. 421.558, found 422.201 (M + 1)+ |
| 1075 | 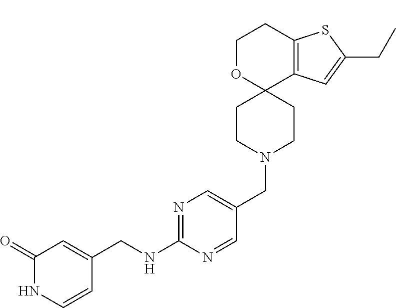 | 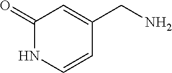 | ESI-MS m/z calc. 451.584, found 452.212 (M + 1)+ |
| 1076 | 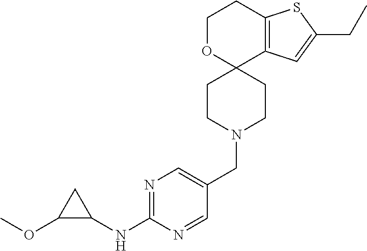 | 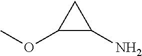 | ESI-MS m/z calc. 414.564, found 415.217 (M + 1)+ |
| 1077 | 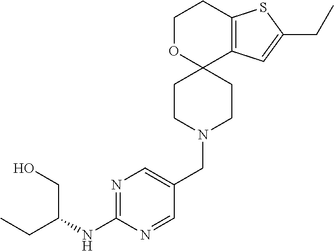 [SMILES: CCc1cc2c(s1)CCOC21CCN(Cc2cnc(N[C@H](CC)CO)nc2)CC1] | 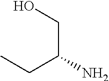 | ESI-MS m/z calc. 416.58, found 417.232 (M + 1)+ |
| 1078 | 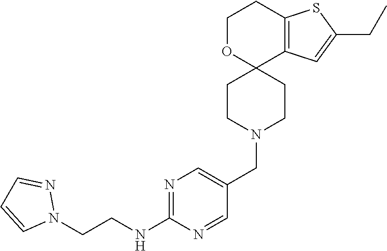 [SMILES: CCc1cc2c(s1)CCOC21CCN(Cc2cnc(NCCn3cccn3)nc2)CC1] | 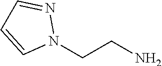 | ESI-MS m/z calc. 438.589, found 439.228 (M + 1)+ |

### Compound 1079

### 1-(4-((2′-ethyl-6′,7′-dihydrospiro[piperidine-4,4′-thieno[3,2-c]pyran]-1-yl)methyl)-1H-1,2,3-triazol-1-yl)-3-methylbutane-2,3-diol (1079)

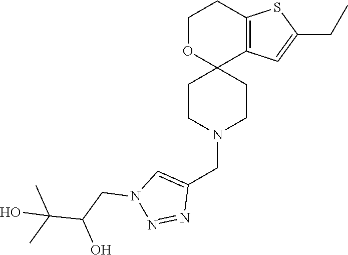 [SMILES: CCc1cc2c(s1)CCOC21CCN(Cc2cn(CC(O)C(C)(C)O)nn2)CC1]

### Step 1: Synthesis of 2-ethyl-1′-prop-2-ynyl-spiro[6,7-dihydrothieno[3,2-c]pyran-4,4′-piperidine](S115)

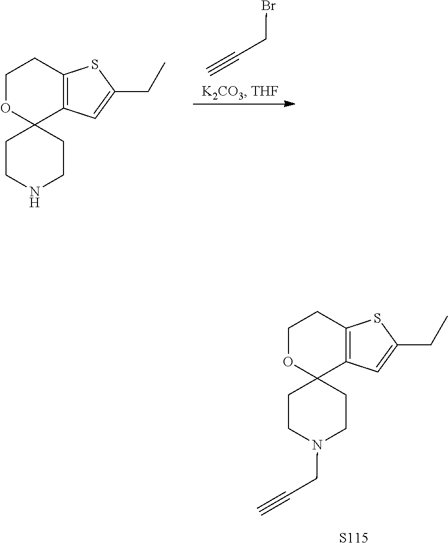

A mixture of 2-ethylspiro[6,7-dihydrothieno[3,2-c]pyran-4,4′-piperidine](2500 mg, 10.53 mmol) and potassium carbonate (1.5 g, 10.85 mmol) in tetrahydrofuran (50 mL) was heated to 50° C. and stirred. At this time, propargyl bromide (1.735 g, 11.67 mmol) in toluene was added and the mixture was stirred at this temperature. After 4 hours, the mixture was cooled to room temperature and diluted with water (100 mL) and EtOAc (100 mL). The layers were separated, and the organic layer was washed with water (2×50 mL). The organic layer was dried with magnesium sulfate, filtered, and concentrated. Purification by flash column chromatography (0-10% MeOH:DCM) provided yield 2-ethyl-1′-prop-2-ynyl-spiro[6,7-dihydrothieno[3,2-c]pyran-4,4′-piperidine](S115) (1072 mg, 36%)¹H NMR (400 MHz, Chloroform-d) δ 6.45 (d, J=1.1 Hz, 1H), 3.91 (t, J=5.4 Hz, 2H), 3.31 (d, J=2.5 Hz, 2H), 2.85-2.65 (m, 6H), 2.65-2.49 (m, 2H), 2.26 (t, J=2.4 Hz, 1H), 2.05-1.92 (m, 2H), 1.92-1.81 (m, 2H), 1.26 (t, J=7.5 Hz, 3H). ESI-MS m/z calc. 275.1344, found 276.43 (M+1)⁺.

### Step 2: Synthesis of 1-(4-((2′-ethyl-6′,7′-dihydrospiro[piperidine-4,4′-thieno[3,2-c]pyran]-1-yl)methyl)-1H-1,2,3-triazol-1-yl)-3-methylbutane-2,3-diol (1079)

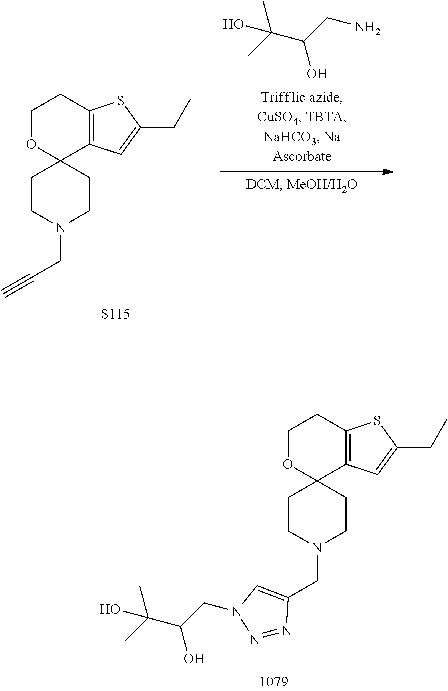

To a mixture of 1-amino-3-methylbutane-2,3-diol (10 mg, 0.084 mmol) in MeOH (600 μL) was added CuSO4 (0.15 mg) in water (75 μL) followed by sodium bicarbonate (6.5 mg, 0.08 mmol) in water (75 μL) and a solution of triflic azide (0.25 mL of 0.452 M) in DCM. This mixture was stirred for 1 hour. After 1 hour, 2-ethyl-1′-prop-2-ynyl-spiro[6,7-dihydrothieno[3,2-c]pyran-4,4′-piperidine] (10 mg, 0.036 mmol) in MeOH (100 μL), sodium ascorbate (7 mg, 0.039 mmol) in water (50 μL) and TBTA (1 mg, 0.002 mmol) in MeOH (100 μL), and the mixture was heated to 50° C. and stirred overnight. At this time, the mixture was dried and rediluted in DMSO for reverse phase HPLC purification with TFA modifier. The product-containing fractions were diluted in DCM and washed with sat. sodium bicarbonate to remove the TFA salt. The organic layers were dried to yield 1-(4-((2′-ethyl-6′,7′-dihydrospiro[piperidine-4,4′-thieno[3,2-c]pyran]-1-yl)methyl)-1H-1,2,3-triazol-1-yl)-3-methylbutane-2,3-diol (11.8 mg, 61%). ¹H NMR (400 MHz, DMSO-d6) δ 9.97 (s, 1H), 8.23 (s, 1H), 6.43 (s, 1H), 5.20 (s, 1H), 4.84-4.59 (m, 2H), 4.51 (s, 2H), 4.21 (dd, J=13.8, 10.0 Hz, 1H), 3.87 (t, J=5.3 Hz, 2H), 3.54 (d, J=9.9 Hz, 1H), 3.34 (s, 2H), 3.18 (d, J=10.2 Hz, 2H), 2.82-2.64 (m, 4H), 2.10 (t, J=13.5 Hz, 2H), 1.98 (d, J=14.4 Hz, 2H), 1.20 (t, J=7.5 Hz, 3H), 1.16 (s, 3H), 1.11 (s, 3H). ESI-MS m/z calc. 420.22, found 421.227 (M+1)⁺.

### Compounds 1080-1125, 1183

Compounds 1080-1125 and compound 1183 (see Table 52) were prepared from S115 using the appropriate amines employing the same method as described for compound 1079.

TABLE 52
Structure and physicochemical data for compounds 1080-1125, 1183

| | | | LCMS |
|---|---|---|---|
| Compd | Product | Amine | m/z [M + H]+ |
| 1080 | 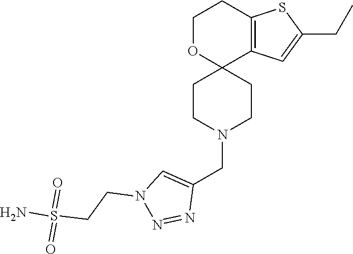 [SMILES: CCc1cc2c(s1)CCOC21CCN(Cc2cn(CCS(N)(=O)=O)nn2)CC1] | 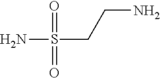 | ESI-MS m/z calc. 425.156, found 426.163 (M + 1)+ |
| 1081 | 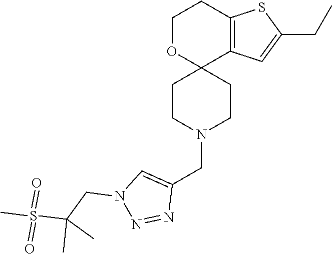 [SMILES: CCc1cc2c(s1)CCOC21CCN(Cc2cn(CC(C)(C)S(C)(=O)=O)nn2)CC1] | 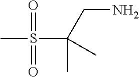 | ESI-MS m/z calc. 452.192, found 453.199 (M + 1)+ |
| 1082 | 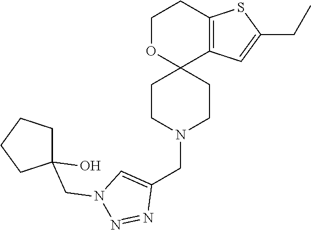 [SMILES: CCc1cc2c(s1)CCOC21CCN(Cc2cn(CC3(O)CCCC3)nn2)CC1] | 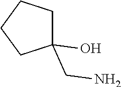 | ESI-MS m/z calc. 416.225, found 417.232 (M + 1)+ |
| 1083 | 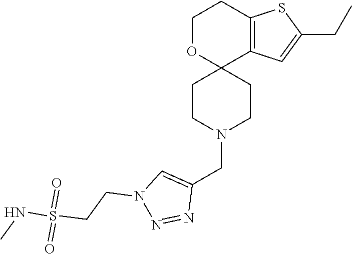 [SMILES: CCc1cc2c(s1)CCOC21CCN(Cc2cn(CCS(=O)(=O)NC)nn2)CC1] | 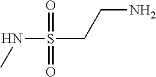 | ESI-MS m/z calc. 439.171, found 440.179 (M + 1)+ |
| 1084 | 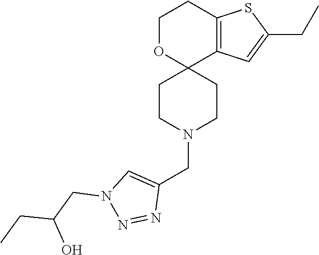 [SMILES: CCc1cc2c(s1)CCOC21CCN(Cc2cn(CC(O)CC)nn2)CC1] | 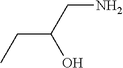 | ESI-MS m/z calc. 390.209, found 391.216 (M + 1)+ |
| 1085 | 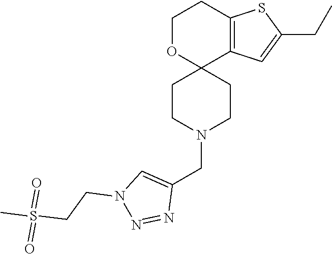 [SMILES: CCc1cc2c(s1)CCOC21CCN(Cc2cn(CCS(C)(=O)=O)nn2)CC1] | 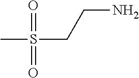 | ESI-MS m/z calc. 424.16, found 425.168 (M + 1)+ |
| 1086 | 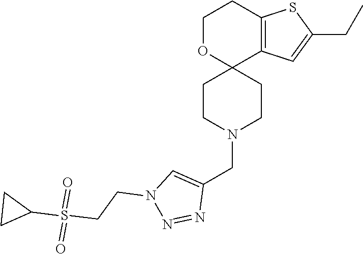 [SMILES: CCc1cc2c(s1)CCOC21CCN(Cc2cn(CCS(=O)(=O)C3CC3)nn2)CC1] | 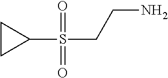 | ESI-MS m/z calc. 450.176, found 451.183 (M + 1)+ |
| 1087 | 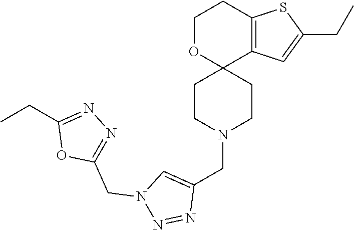 [SMILES: CCc1nnc(Cn2cc(CN3CCC4(CC3)OCCc3sc(CC)cc34)nn2)o1] | 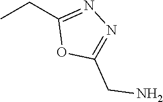 | ESI-MS m/z calc. 428.199, found 429.207 (M + 1)+ |
| 1088 | 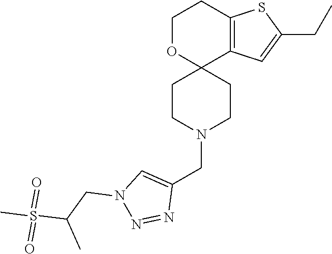 [SMILES: CCc1cc2c(s1)CCOC21CCN(Cc2cn(CC(C)S(C)(=O)=O)nn2)CC1] | 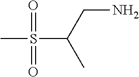 | ESI-MS m/z calc. 438.176, found 439.184 (M + 1)+ |
| 1089 | 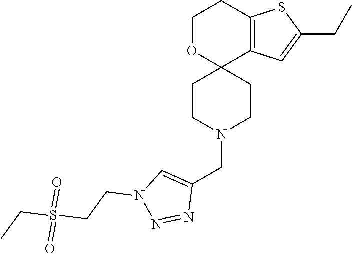 [SMILES: CCc1cc2c(s1)CCOC21CCN(Cc2cn(CCS(=O)(=O)CC)nn2)CC1] | 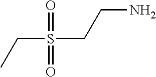 | ESI-MS m/z calc. 438.176, found 439.183 (M + 1)+ |
| 1090 | 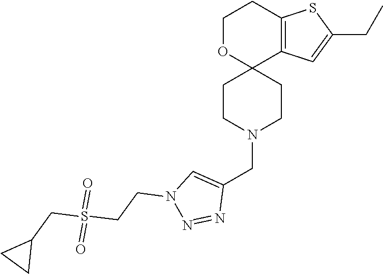 [SMILES: CCc1cc2c(s1)CCOC21CCN(Cc2cn(CCS(=O)(=O)CC3CC3)nn2)CC1] | 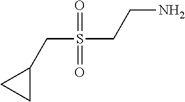 | ESI-MS m/z calc. 464.192, found 465.199 (M + 1)+ |
| 1091 | 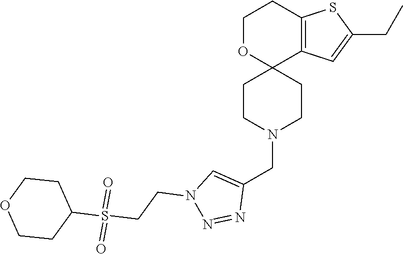 [SMILES: CCc1cc2c(s1)CCOC21CCN(Cc2cn(CCS(=O)(=O)C3CCOCC3)nn2)CC1] | 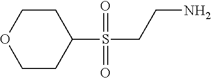 [SMILES: NCCS(=O)(=O)C1CCOCC1] | ESI-MS m/z calc. 494.202, found 495.21 (M + 1)+ |
| 1092 | 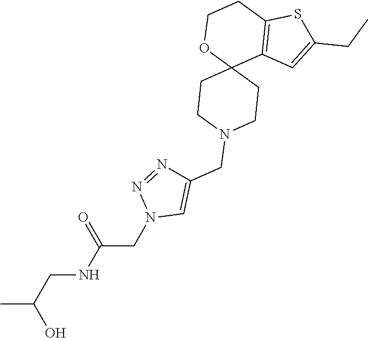 [SMILES: CCc1cc2c(s1)CCOC21CCN(Cc2cn(CC(=O)NCC(C)O)nn2)CC1] | 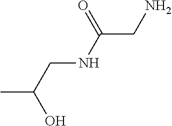 | ESI-MS m/z calc. 433.215, found 434.222 (M + 1)+ |
| 1093 | 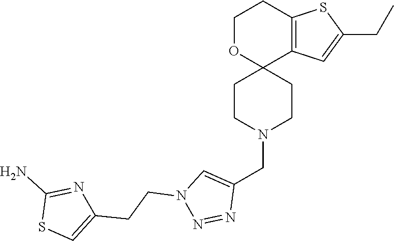 [SMILES: CCc1cc2c(s1)CCOC21CCN(Cc2cn(CCc3csc(N)n3)nn2)CC1] | 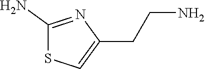 | ESI-MS m/z calc. 444.177, found 445.184 (M + 1)+ |
| 1094 | 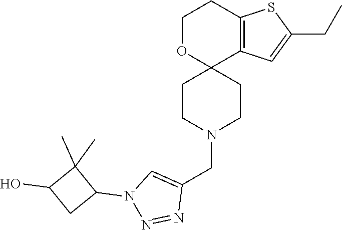 [SMILES: CCc1cc2c(s1)CCOC21CCN(Cc2cn(C3CC(O)C3(C)C)nn2)CC1] | 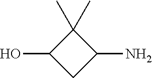 | ESI-MS m/z calc. 416.225, found 417.232 (M + 1)+ |
| 1095 | 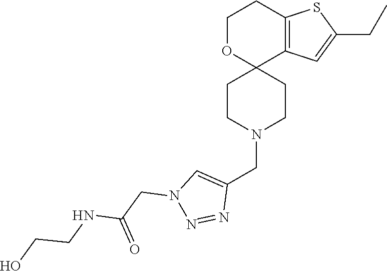 [SMILES: CCc1cc2c(s1)CCOC21CCN(Cc2cn(CC(=O)NCCO)nn2)CC1] | 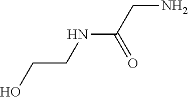 | ESI-MS m/z calc. 419.199, found 420.206 (M + 1)+ |
| 1096 | 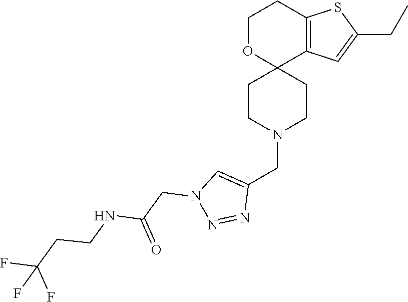 [SMILES: CCc1cc2c(s1)CCOC21CCN(Cc2cn(CC(=O)NCCC(F)(F)F)nn2)CC1] | 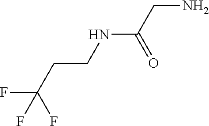 [SMILES: NCC(=O)NCCC(F)(F)F] | ESI-MS m/z calc. 471.192, found 472.199 (M + 1)+ |
| 1097 | 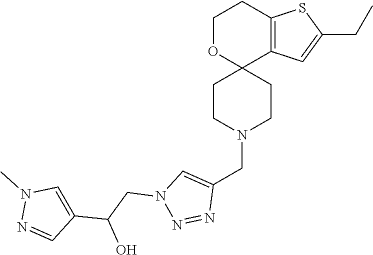 [SMILES: CCc1cc2c(s1)CCOC21CCN(Cc2cn(CC(O)c3cnn(C)c3)nn2)CC1] | 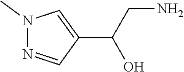 | ESI-MS m/z calc. 442.215, found 443.223 (M + 1)+ |
| 1098 | 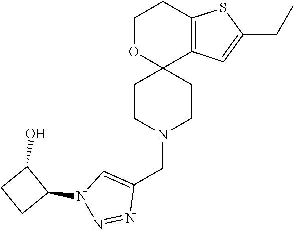 [SMILES: CCc1cc2c(s1)CCOC21CCN(Cc2cn([C@H]3CC[C@@H]3O)nn2)CC1] | 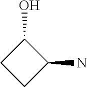 | ESI-MS m/z calc. 388.193, found 389.2 (M + 1)+ |
| 1099 | 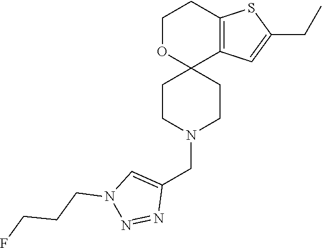 [SMILES: CCc1cc2c(s1)CCOC21CCN(Cc2cn(CCCF)nn2)CC1] | 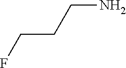 | ESI-MS m/z calc. 378.189, found 379.196 (M + 1)+ |
| 1100 | 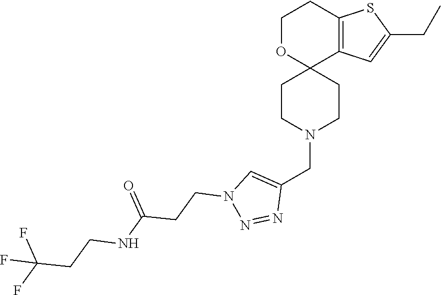 [SMILES: CCc1cc2c(s1)CCOC21CCN(Cc2cn(CCC(=O)NCCC(F)(F)F)nn2)CC1] | 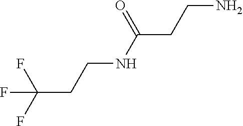 [SMILES: NCCC(=O)NCCC(F)(F)F] | ESI-MS m/z calc. 485.207, found 486.214 (M + 1)+ |
| 1101 | 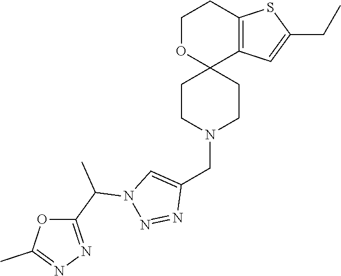 [SMILES: CCc1cc2c(s1)CCOC21CCN(Cc2cn(C(C)c3nnc(C)o3)nn2)CC1] | 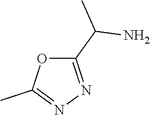 | ESI-MS m/z calc. 428.199, found 429.207 (M + 1)+ |
| 1102 | 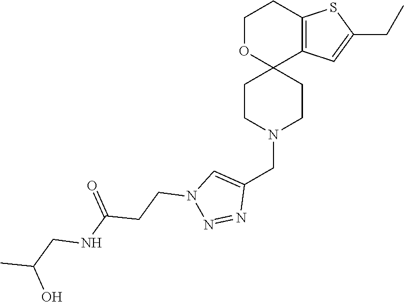 [SMILES: CCc1cc2c(s1)CCOC21CCN(Cc2cn(CCC(=O)NCC(C)O)nn2)CC1] | 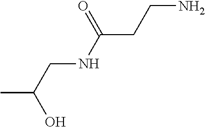 | ESI-MS m/z calc. 447.23, found 448.238 (M + 1)+ |
| 1103 | 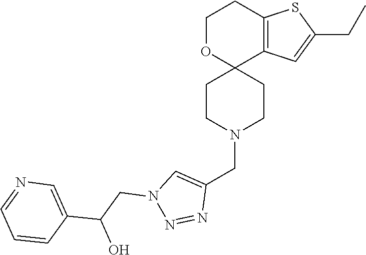 [SMILES: CCc1cc2c(s1)CCOC21CCN(Cc2cn(CC(O)c3cccnc3)nn2)CC1] | 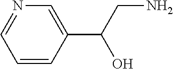 | ESI-MS m/z calc. 439.204, found 440.212 (M + 1)+ |
| 1104 | 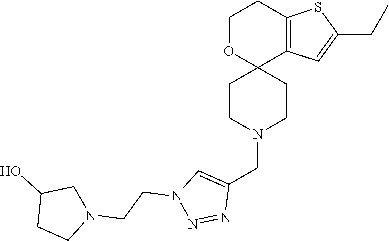 [SMILES: CCc1cc2c(s1)CCOC21CCN(Cc2cn(CCN3CCC(O)C3)nn2)CC1] | 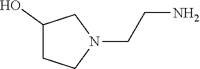 | ESI-MS m/z calc. 431.235, found 432.243 (M + 1)+ |
| 1105 | 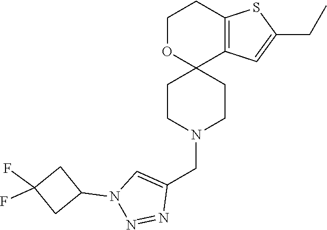 [SMILES: CCc1cc2c(s1)CCOC21CCN(Cc2cn(C3CC(F)(F)C3)nn2)CC1] | 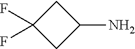 | ESI-MS m/z calc. 408.18, found 409.187 (M + 1)+ |
| 1106 | 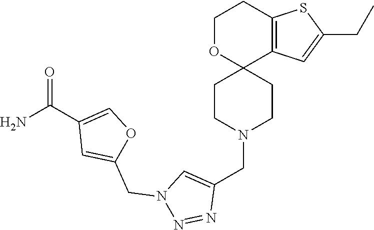 [SMILES: CCc1cc2c(s1)CCOC21CCN(Cc2cn(Cc3cc(C(N)=O)co3)nn2)CC1] | 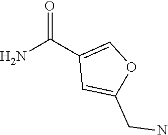 | ESI-MS m/z calc. 441.183, found 442.191 (M + 1)+ |
| 1107 | 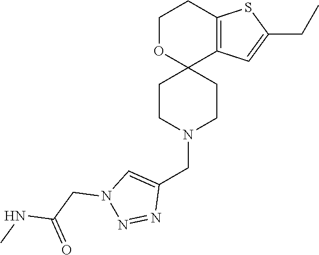 [SMILES: CCc1cc2c(s1)CCOC21CCN(Cc2cn(CC(=O)NC)nn2)CC1] | 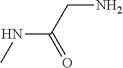 | ESI-MS m/z calc. 389.189, found 390.196 (M + 1)+ |
| 1108 | 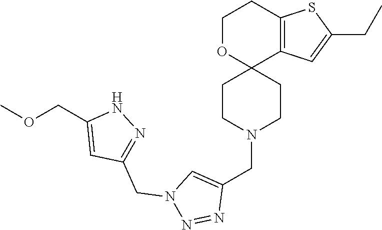 [SMILES: CCc1cc2c(s1)CCOC21CCN(Cc2cn(Cc3cc(COC)[nH]n3)nn2)CC1] | 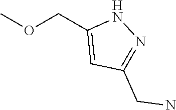 | ESI-MS m/z calc. 442.215, found 443.223 (M + 1)+ |
| 1109 | 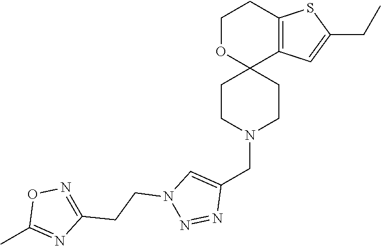 [SMILES: CCc1cc2c(s1)CCOC21CCN(Cc2cn(CCc3noc(C)n3)nn2)CC1] | 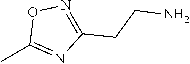 | ESI-MS m/z calc. 428.199, found 429.207 (M + 1)+ |
| 1110 | 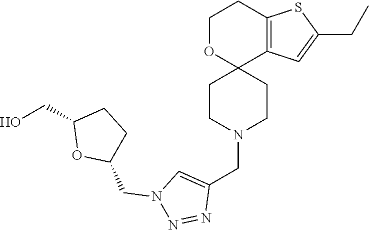 [SMILES: CCc1cc2c(s1)CCOC21CCN(Cc2cn(C[C@H]3CC[C@@H](CO)O3)nn2)CC1] | 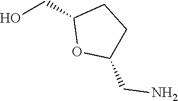 | ESI-MS m/z calc. 432.22, found 433.227 (M + 1)+ |
| 1111 | 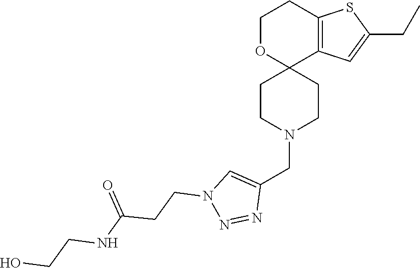 [SMILES: CCc1cc2c(s1)CCOC21CCN(Cc2cn(CCC(=O)NCCO)nn2)CC1] | 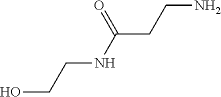 | ESI-MS m/z calc. 433.215, found 434.222 (M + 1)+ |
| 1112 | 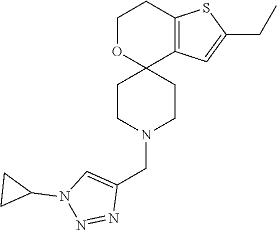 [SMILES: CCc1cc2c(s1)CCOC21CCN(Cc2cn(C3CC3)nn2)CC1] | 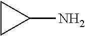 | ESI-MS m/z calc. 358.183, found 359.19 (M + 1)+ |
| 1113 | 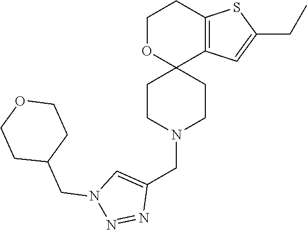 [SMILES: CCc1cc2c(s1)CCOC21CCN(Cc2cn(CC3CCOCC3)nn2)CC1] | 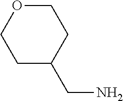 | ESI-MS m/z calc. 416.225, found 417.232 (M + 1)+ |
| 1114 | 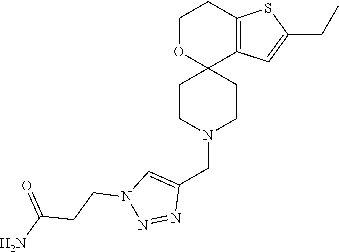 [SMILES: CCc1cc2c(s1)CCOC21CCN(Cc2cn(CCC(N)=O)nn2)CC1] | 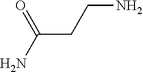 | ESI-MS m/z calc. 389.189, found 390.196 (M + 1)+ |
| 1115 | 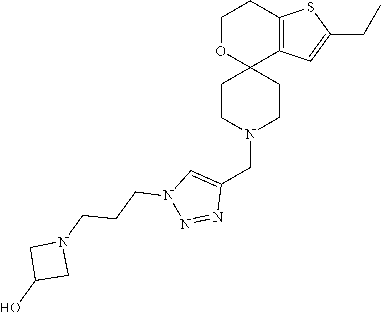 [SMILES: CCc1cc2c(s1)CCOC21CCN(Cc2cn(CCCN3CC(O)C3)nn2)CC1] | 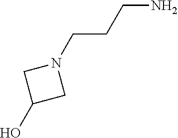 | ESI-MS m/z calc. 431.235, found 432.243 (M + 1)+ |
| 1116 | 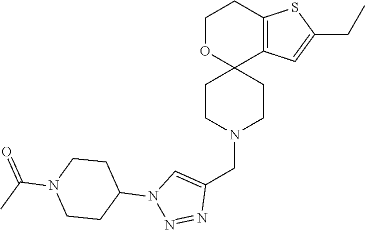 [SMILES: CCc1cc2c(s1)CCOC21CCN(Cc2cn(C3CCN(C(C)=O)CC3)nn2)CC1] | 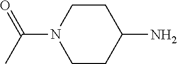 | ESI-MS m/z calc. 443.235, found 444.243 (M + 1)+ |
| 1117 | 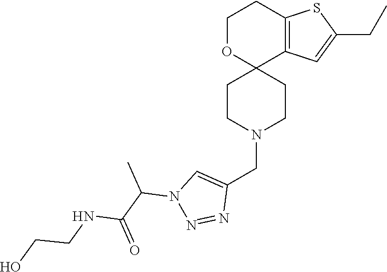 [SMILES: CCc1cc2c(s1)CCOC21CCN(Cc2cn(C(C)C(=O)NCCO)nn2)CC1] | 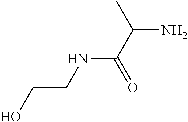 | ESI-MS m/z calc. 433.215, found 434.222 (M + 1)+ |
| 1118 | 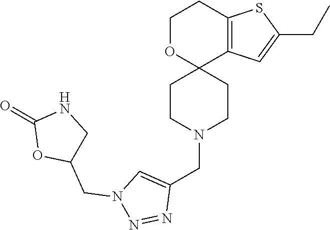 [SMILES: CCc1cc2c(s1)CCOC21CCN(Cc2cn(CC3CNC(=O)O3)nn2)CC1] | 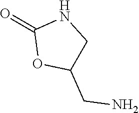 | ESI-MS m/z calc. 417.183, found 418.191 (M + 1)+ |
| 1119 | 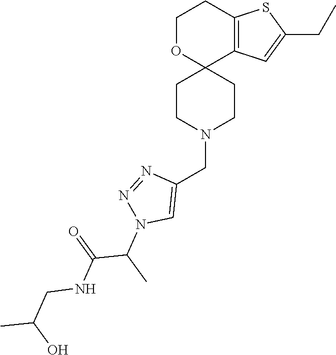 [SMILES: CCc1cc2c(s1)CCOC21CCN(Cc2cn(C(C)C(=O)NCC(C)O)nn2)CC1] | 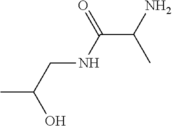 | ESI-MS m/z calc. 447.23, found 448.238 (M + 1)+ |
| 1120 | 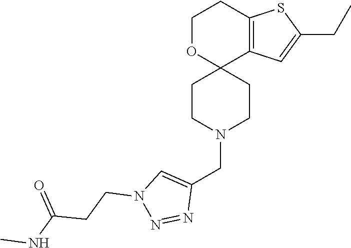 [SMILES: CCc1cc2c(s1)CCOC21CCN(Cc2cn(CCC(=O)NC)nn2)CC1] | 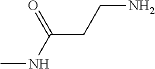 | ESI-MS m/z calc. 403.204, found 404.211 (M + 1)+ |
| 1121 | 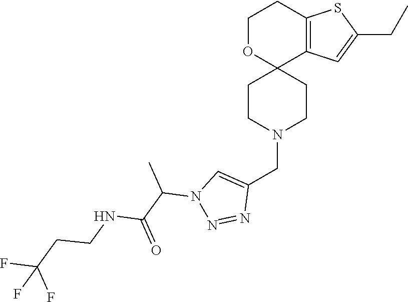 [SMILES: CCc1cc2c(s1)CCOC21CCN(Cc2cn(C(C)C(=O)NCCC(F)(F)F)nn2)CC1] | 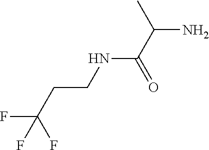 | ESI-MS m/z calc. 485.207, found 486.215 (M + 1)+ |
| 1122 | 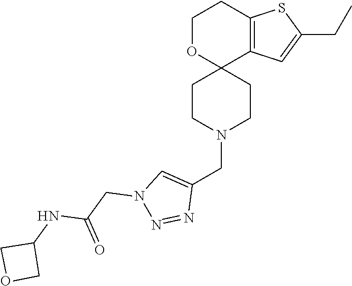 [SMILES: CCc1cc2c(s1)CCOC21CCN(Cc2cn(CC(=O)NC3COC3)nn2)CC1] | 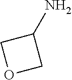 | ESI-MS m/z calc. 431.199, found 432.207 (M + 1)+ |
| 1123 | 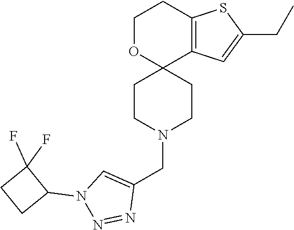 [SMILES: CCc1cc2c(s1)CCOC21CCN(Cc2cn(C3CCC3(F)F)nn2)CC1] | 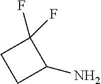 | ESI-MS m/z calc. 408.18, found 409.187 (M + 1)+ |
| 1124 | 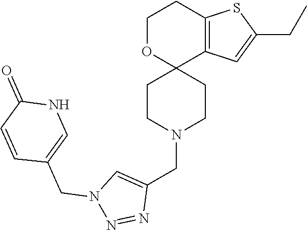 [SMILES: CCc1cc2c(s1)CCOC21CCN(Cc2cn(Cc3ccc(=O)[nH]c3)nn2)CC1] | 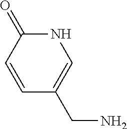 | ESI-MS m/z calc. 425.189, found 426.196 (M + 1)+ |
| 1125 | 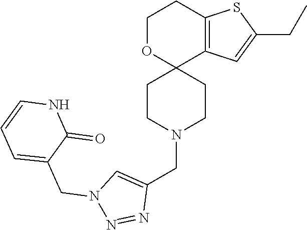 [SMILES: CCc1cc2c(s1)CCOC21CCN(Cc2cn(Cc3ccc[nH]c3=O)nn2)CC1] | 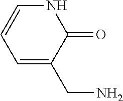 | ESI-MS m/z calc. 425.189, found 426.196 (M + 1)+ |
| 1183 | 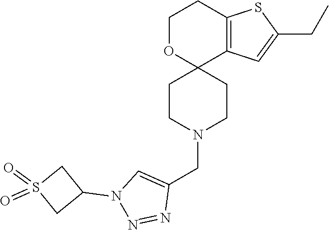 [SMILES: CCc1cc2c(s1)CCOC21CCN(Cc2cn(C3CS(=O)(=O)C3)nn2)CC1] | 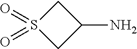 | ESI-MS m/z calc. 422.145, found 423.152 (M + 1)+ |

### Compound 1126

### Synthesis of 2′-ethyl-1-((5-methoxy-1H-pyrrolo[3,2-b]pyridin-2-yl)methyl)-6′,7′-dihydrospiro[piperidine-4,4′-thieno[3,2-c]pyran] (1126)

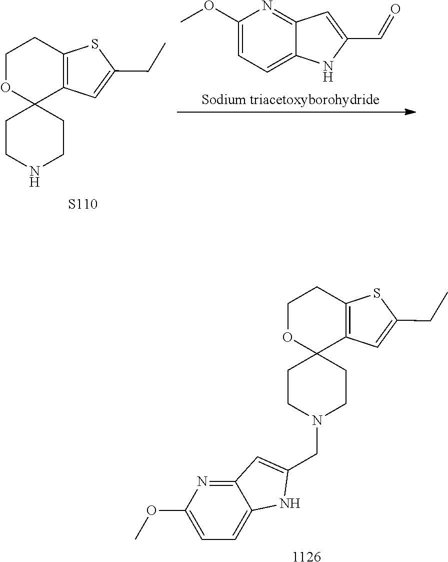

A solution of 2-ethylspiro[6,7-dihydrothieno[3,2-c]pyran-4,4′-piperidine](Trifluoroacetate salt) (15 mg, 0.06 mmol) and 5-methoxy-1H-pyrrolo[3,2-b]pyridine-2-carbaldehyde (16 mg, 0.09 mmol) in dichloromethane (1 mL) was stirred at room temperature for 15 minutes, followed by addition of sodium triacetoxyborohydride (13 mg, 0.06 mmol). The resulting solution was stirred at room temperature overnight, then concentrated, and purified by reverse phase HPLC with 10-90% ACN in Water (TFA modifier) to afford 2′-ethyl-1-((5-methoxy-1H-pyrrolo[3,2-b]pyridin-2-yl)methyl)-6′,7′-dihydrospiro[piperidine-4,4′-thieno[3,2-c]pyran] (15.2 mg, 62%). ¹H NMR (400 MHz, DMSO-d6) δ 7.80 (d, J=8.8 Hz, 1H), 6.70-6.60 (m, 2H), 6.44 (s, 1H), 4.53 (s, 2H), 3.86 (s, 3H), 3.34 (d, J=12.1 Hz, 2H), 3.18 (d, J=12.4 Hz, 2H), 2.77-2.66 (m, 4H), 2.11 (t, J=13.1 Hz, 2H), 1.99 (d, J=14.4 Hz, 2H), 1.19 (t, J=7.5 Hz, 3H). ESI-MS m/z calc. 397.18, found 398.32 (M+1)⁺.

### Compounds 1127-1157, S116

Compounds 1127-1157 and compound S116 (see Table 53) were prepared from S110 using the appropriate aldehydes employing the same method as described for compound 1126.

TABLE 53
Structure and physicochemical data for compounds 1127-1157, S116

| | | | LCMS m/z |
|---|---|---|---|
| Cmpd | Product | Aldehyde | [M + H]+ |
| 1127 | 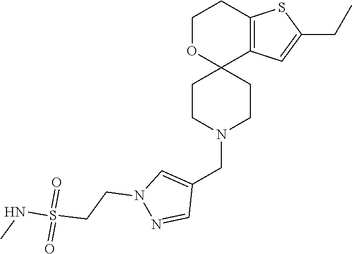 [SMILES: CCc1cc2c(s1)CCOC21CCN(Cc2cnn(CCS(=O)(=O)NC)c2)CC1] | 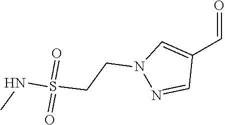 | ESI-MS m/z calc. 438.176, found 439.183 (M + 1)+ |
| 1128 | 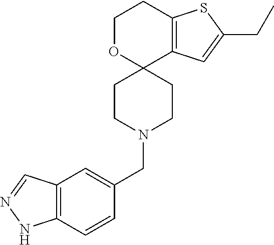 [SMILES: CCc1cc2c(s1)CCOC21CCN(Cc2ccc3[nH]ncc3c2)CC1] | 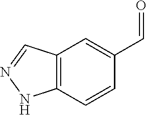 [SMILES: O=Cc1ccc2[nH]ncc2c1] | ESI-MS m/z calc. 367.172, found 368.179 (M + 1)+ |
| 1129 | 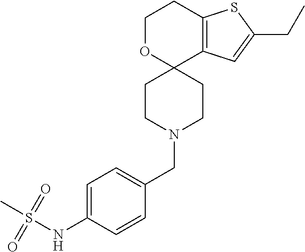 [SMILES: CCc1cc2c(s1)CCOC21CCN(Cc2ccc(NS(C)(=O)=O)cc2)CC1] | 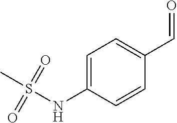 | ESI-MS m/z calc. 420.154, found 421.161 (M + 1)+ |
| 1130 | 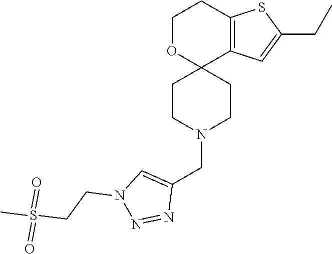 [SMILES: CCc1cc2c(s1)CCOC21CCN(Cc2cn(CCS(C)(=O)=O)nn2)CC1] | 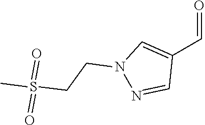 | ESI-MS m/z calc. 423.165, found 424.172 (M + 1)+ |
| 1131 | 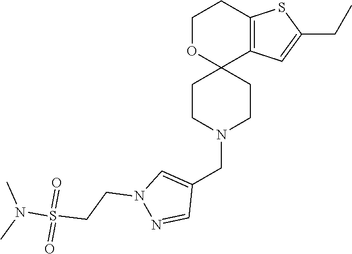 [SMILES: CCc1cc2c(s1)CCOC21CCN(Cc2cnn(CCS(=O)(=O)N(C)C)c2)CC1] | 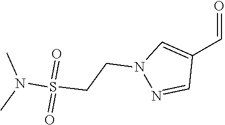 | ESI-MS m/z calc. 452.192, found 453.199 (M + 1)+ |
| 1132 | 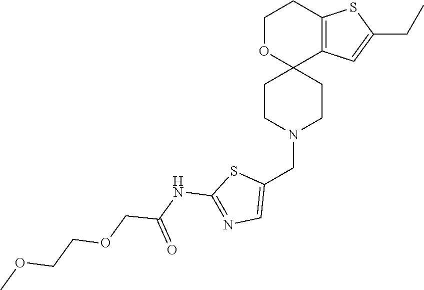 [SMILES: CCc1cc2c(s1)CCOC21CCN(Cc2cnc(NC(=O)COCCOC)s2)CC1] | 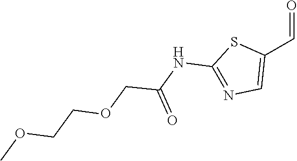 | ESI-MS m/z calc. 465.176, found 466.184 (M + 1)+ |
| 1133 | 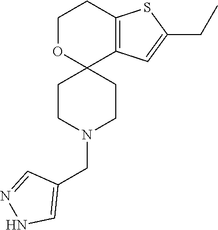 [SMILES: CCc1cc2c(s1)CCOC21CCN(Cc2cn[nH]c2)CC1] | 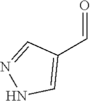 | ESI-MS m/z calc. 317.156, found 318.164 (M + 1)+ |
| 1134 | 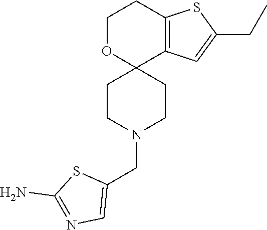 [SMILES: CCc1cc2c(s1)CCOC21CCN(Cc2cnc(N)s2)CC1] | 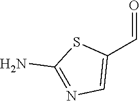 | ESI-MS m/z calc. 349.128, found 350.136 (M + 1)+ |
| 1135 | 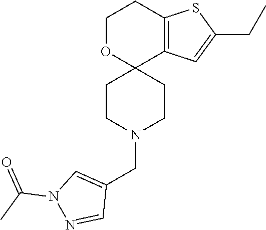 [SMILES: CCc1cc2c(s1)CCOC21CCN(Cc2cnn(C(C)=O)c2)CC1] | 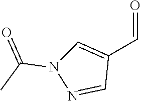 | ESI-MS m/z calc. 359.167, found 360.174 (M + 1)+ |
| 1136 | 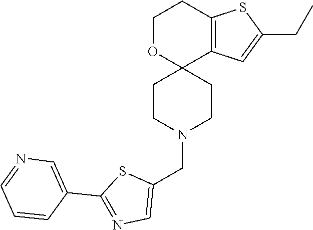 [SMILES: CCc1cc2c(s1)CCOC21CCN(Cc2cnc(-c3cccnc3)s2)CC1] | 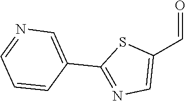 [SMILES: O=Cc1cnc(-c2cccnc2)s1] | ESI-MS m/z calc. 411.144, found 412.151 (M + 1)+ |
| 1137 | 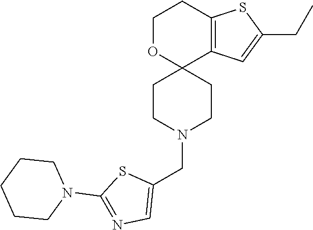 [SMILES: CCc1cc2c(s1)CCOC21CCN(Cc2cnc(N3CCCCC3)s2)CC1] | 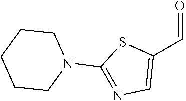 [SMILES: O=Cc1cnc(N2CCCCC2)s1] | ESI-MS m/z calc. 417.191, found 418.198 (M + 1)+ |
| 1138 | 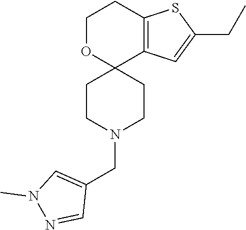 [SMILES: CCc1cc2c(s1)CCOC21CCN(Cc2cnn(C)c2)CC1] | 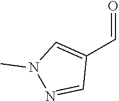 | ESI-MS m/z calc. 331.172, found 332.18 (M + 1)+ |
| 1139 | 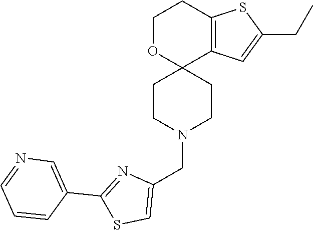 [SMILES: CCc1cc2c(s1)CCOC21CCN(Cc2csc(-c3cccnc3)n2)CC1] | 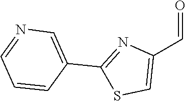 [SMILES: O=Cc1csc(-c2cccnc2)n1] | ESI-MS m/z calc. 411.144, found 412.151 (M + 1)+ |
| 1140 | 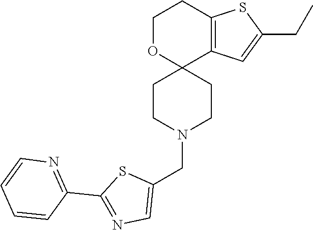 [SMILES: CCc1cc2c(s1)CCOC21CCN(Cc2cnc(-c3ccccn3)s2)CC1] | 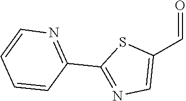 [SMILES: O=Cc1cnc(-c2ccccn2)s1] | ESI-MS m/z calc. 411.144, found 412.151 (M + 1)+ |
| 1141 | 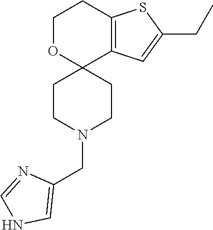 [SMILES: CCc1cc2c(s1)CCOC21CCN(Cc2c[nH]cn2)CC1] | 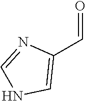 | ESI-MS m/z calc. 317.156, found 318.163 (M + 1)+ |
| 1142 | 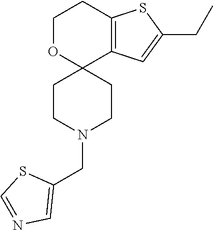 [SMILES: CCc1cc2c(s1)CCOC21CCN(Cc2cncs2)CC1] | 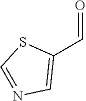 | ESI-MS m/z calc. 334.117, found 335.125 (M + 1)+ |
| 1143 | 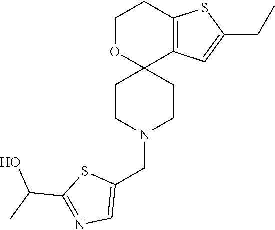 [SMILES: CCc1cc2c(s1)CCOC21CCN(Cc2cnc(C(C)O)s2)CC1] | 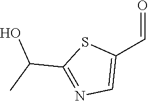 | ESI-MS m/z calc. 378.144, found 379.151 (M + 1)+ |
| 1144 | 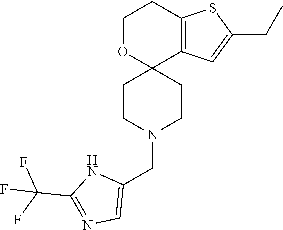 [SMILES: CCc1cc2c(s1)CCOC21CCN(Cc2cnc(C(F)(F)F)[nH]2)CC1] | 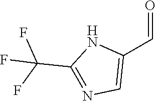 [SMILES: O=Cc1cnc(C(F)(F)F)[nH]1] | ESI-MS m/z calc. 385.144, found 386.151 (M + 1)+ |
| 1145 | 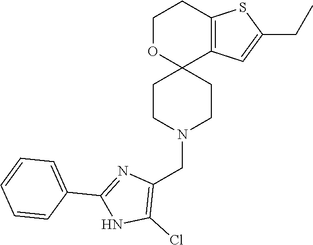 [SMILES: CCc1cc2c(s1)CCOC21CCN(Cc2nc(-c3ccccc3)[nH]c2Cl)CC1] | 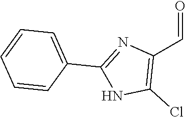 [SMILES: O=Cc1nc(-c2ccccc2)[nH]c1Cl] | ESI-MS m/z calc. 427.149, found 428.156 (M + 1)+ |
| 1146 | 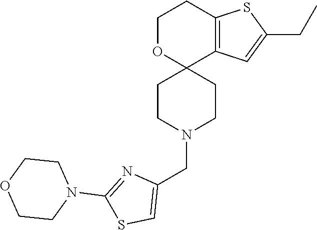 [SMILES: CCc1cc2c(s1)CCOC21CCN(Cc2csc(N3CCOCC3)n2)CC1] | 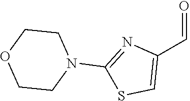 [SMILES: O=Cc1csc(N2CCOCC2)n1] | ESI-MS m/z calc. 419.17, found 420.177 (M + 1)+ |
| 1147 | 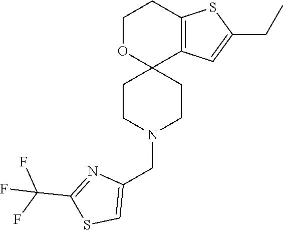 [SMILES: CCc1cc2c(s1)CCOC21CCN(Cc2csc(C(F)(F)F)n2)CC1] | 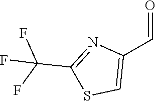 [SMILES: O=Cc1csc(C(F)(F)F)n1] | ESI-MS m/z calc. 402.105, found 403.112 (M + 1)+ |
| 1148 | 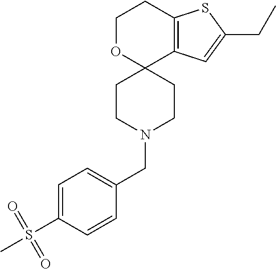 [SMILES: CCc1cc2c(s1)CCOC21CCN(Cc2ccc(S(C)(=O)=O)cc2)CC1] | 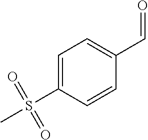 | ESI-MS m/z calc. 405.143, found 406.151 (M + 1)+ |
| 1149 | 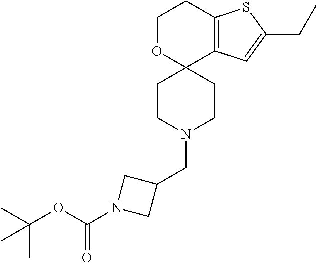 [SMILES: CCc1cc2c(s1)CCOC21CCN(CC2CN(C(=O)OC(C)(C)C)C2)CC1] | 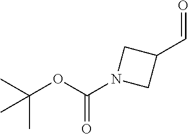 | ESI-MS m/z calc. 406.229, found 407.236 (M + 1)+ |
| 1150 | 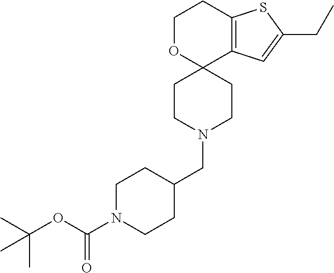 [SMILES: CCc1cc2c(s1)CCOC21CCN(CC2CCN(C(=O)OC(C)(C)C)CC2)CC1] | 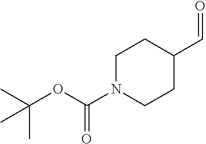 | ESI-MS m/z calc. 434.26, found 435.268 (M + 1)+ |
| 1151 | 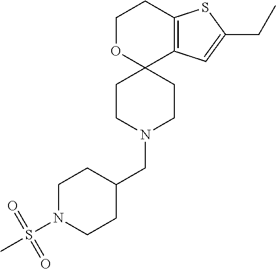 [SMILES: CCc1cc2c(s1)CCOC21CCN(CC2CCN(S(C)(=O)=O)CC2)CC1] | 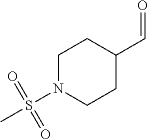 | ESI-MS m/z calc. 412.185, found 413.193 (M + 1)+ |
| 1152 | 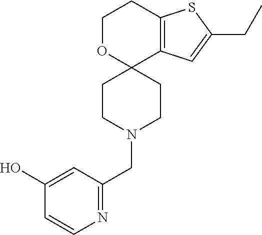 [SMILES: CCc1cc2c(s1)CCOC21CCN(Cc2cc(O)ccn2)CC1] | 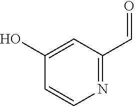 | ESI-MS m/z calc. 344.156, found 345.163 (M + 1)+ |
| 1153 | 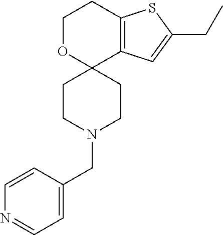 [SMILES: CCc1cc2c(s1)CCOC21CCN(Cc2ccncc2)CC1] | 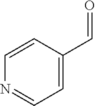 | ESI-MS m/z calc. 328.161, found 329.168 (M + 1)+ |
| 1154 | 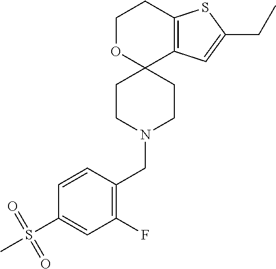 [SMILES: CCc1cc2c(s1)CCOC21CCN(Cc2ccc(S(C)(=O)=O)cc2F)CC1] | 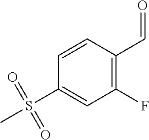 | ESI-MS m/z calc. 423.134, found 424.141 (M + 1)+ |
| 1155 | 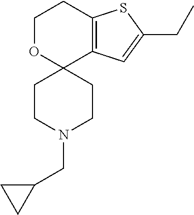 [SMILES: CCc1cc2c(s1)CCOC21CCN(CC2CC2)CC1] | 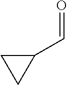 | ESI-MS m/z calc. 291.166, found 292.173 (M + 1)+ |
| 1156 | 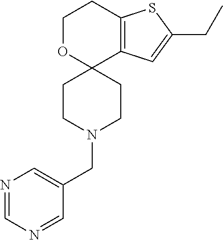 [SMILES: CCc1cc2c(s1)CCOC21CCN(Cc2cncnc2)CC1] | 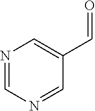 | ESI-MS m/z calc. 329.156, found 330.164 (M + 1)+ |
| 1157 | 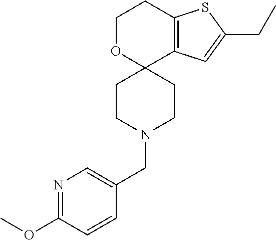 [SMILES: CCc1cc2c(s1)CCOC21CCN(Cc2ccc(OC)nc2)CC1] | 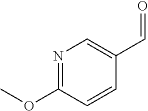 | ESI-MS m/z calc. 358.171, found 359.179 (M + 1)+ |
| 1158 | 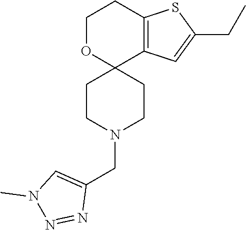 [SMILES: CCc1cc2c(s1)CCOC21CCN(Cc2cn(C)nn2)CC1] | 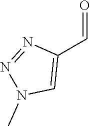 | ESI-MS m/z calc. 332.167, found 333.174 (M + 1)+ |
| S116 | 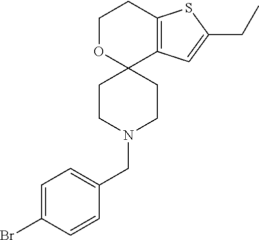 [SMILES: CCc1cc2c(s1)CCOC21CCN(Cc2ccc(Br)cc2)CC1] | 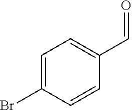 | ESI-MS m/z calc. 406.38 found 406.26 (M + 1)+ |

### Compound 1159

### N-(4-((2′-ethyl-6′,7′-dihydrospiro[piperidine-4,4′-thieno[3,2-c]pyran]-1-yl)methyl)phenyl)-3-hydroxypropane-1-sulfonamide (1159)

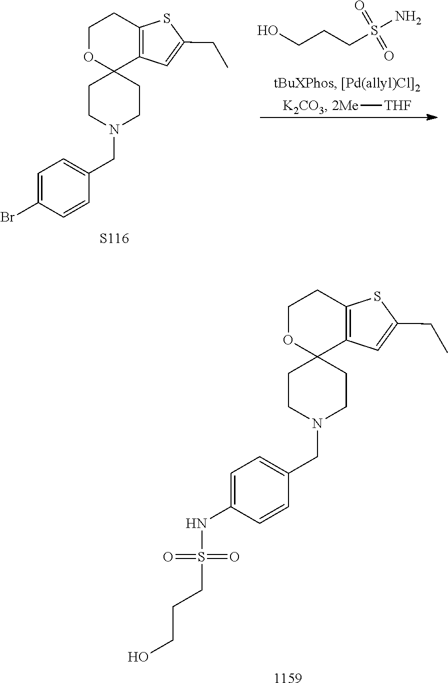

A mixture of 1′-[(4-bromophenyl)methyl]-2-ethyl-spiro[6,7-dihydrothieno[3,2-c]pyran-4,4′-piperidine]S115 (20 mg, 0.05 mmol), 3-hydroxypropane-1-sulfonamide (7.7 mg, 0.05 mmol), K₂CO₃ (14 mg, 0.1 mmol), ditert-butyl-[2-(2,4,6-triisopropylphenyl)phenyl]phosphane (2 mg, 0.005 mmol), and allyl(chloro)palladium (0.45 mmol) in 2-MeTHF (1 mL) was heated to 80° C. The reaction was cooled to room temperature and then diluted with EtOAc and 1 N HCl. The organic layers were separated and concentrated, and then rediluted in DMSO and purified by reverse-phase chromatography (TFA modifier). The product-containing fractions were concentrated to yield N-(4-((2′-ethyl-6′,7′-dihydrospiro[piperidine-4,4′-thieno[3,2-c]pyran]-1-yl)methyl)phenyl)-3-hydroxypropane-1-sulfonamide (7.8 mg, 340).

### Compounds 1160-1164

Compounds 1160-1164 (see Table 54) were prepared from S116 using the appropriate aldehydes employing the same method as described for compound 1159.

TABLE 54
Structure and physicochemical data for compounds 1160-1164

| | | | LCMS m/z |
|---|---|---|---|
| Compd | Product | Amine | [M + H]+ |
| 1161 | 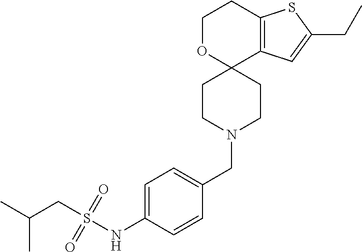 | 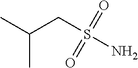 | ESI-MS m/z calc. 478.196, found 479.204 (M + 1)+ |
| 1161 | 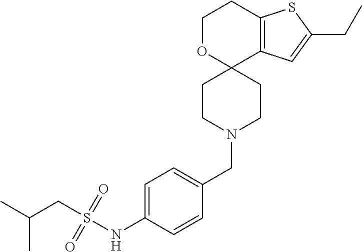 | 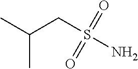 | ESI-MS m/z calc. 478.196, found 479.204 (M + 1)+ |
| 1161 | 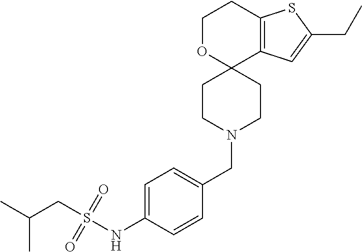 | 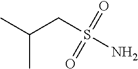 | ESI-MS m/z calc. 478.196, found 479.204 (M + 1)+ |
| 1161 | 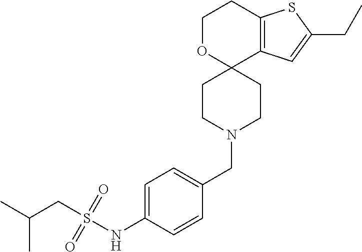 | 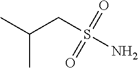 | ESI-MS m/z calc. 478.196, found 479.204 (M + 1)+ |
| 1161 | 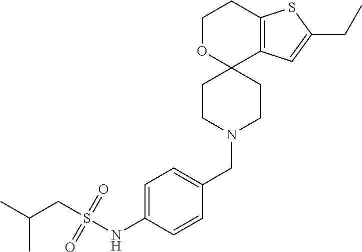 | 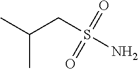 | ESI-MS m/z calc. 478.196, found 479.204 (M + 1)+ |

### Compound 1165

### 2-chloro-1′-(1H-pyrazol-4-ylmethyl)spiro[6,7-dihydrothieno[3,2-c]pyran-4,4′-piperidine](1165)

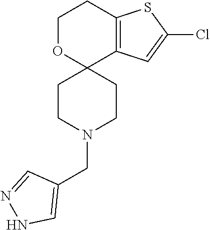 [SMILES: Clc1cc2c(s1)CCOC21CCN(Cc2cn[nH]c2)CC1]

### Step 1: Synthesis of 2-chlorospiro[6,7-dihydrothieno[3,2-c]pyran-4,4′-piperidine](S117)

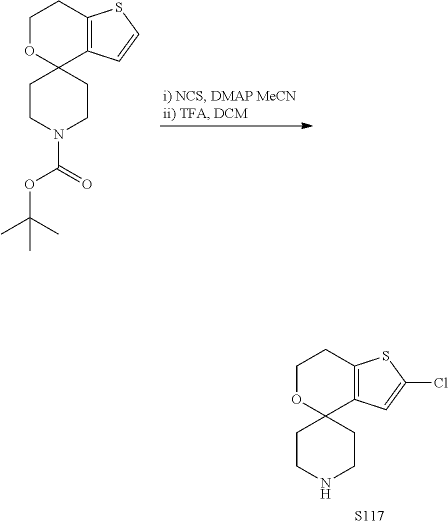 [SMILES: CC(C)(C)OC(=O)N1CCC2(CC1)OCCc1sccc12 ; Clc1cc2c(s1)CCOC21CCNCC1 ; "S117"]

Tert-butyl spiro[6,7-dihydrothieno[3,2-c]pyran-4,4′-piperidine]-1′-carboxylate (5300 mg, 17.13 mmol) was dissolved in CH₃CN (100 mL) and DMAP (180 mg, 1.473 mmol) and N-chlorosuccinimide (2.6 g, 19.47 mmol) added. The reaction mixture was heated to 65° C. for 5 hours, concentrated, taken up in DCM (10 mL), filtered and purified by flash column chromatography (0-50% EtOAc/heptane) to provide the product as a white solid. The resulting tert-butyl 2-chlorospiro[6,7-dihydrothieno[3,2-c]pyran-4,4′-piperidine]-1′-carboxylate was dissolved in DCM (100 mL) followed by the addition of TFA (20 mL, 259.6 mmol) under nitrogen and stirred overnight. The reaction mixture was dried to a crude residue, dissolved in DCM, and neutralized with saturated bicarbonate solution until gas evolution ceased. The organic layer was concentrated to yield 2-chlorospiro[6,7-dihydrothieno[3,2-c]pyran-4,4′-piperidine](2.84 g, 42%) ¹H NMR (300 MHz, Chloroform-d) δ 6.69 (s, 1H), 3.96 (m, 2H), 3.44-3.20 (m, 4H), 2.77 (t, J=5.3 Hz, 2H), 2.28-1.92 (m, 4H).

### Step 2: Synthesis of 2-chloro-1′-(1H-pyrazol-4-ylmethyl)spiro[6,7-dihydrothieno[3,2-c]pyran-4,4′-piperidine](1165)

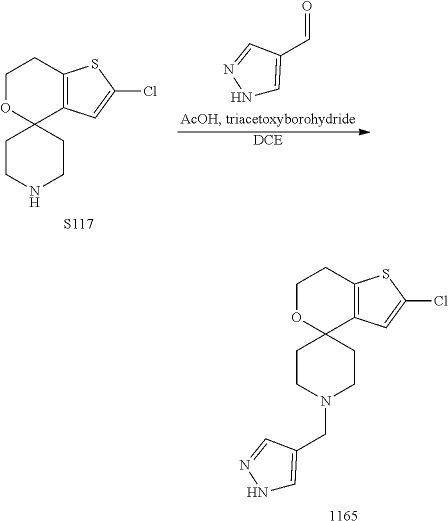 [SMILES: Clc1cc2c(s1)CCOC21CCNCC1 ; O=Cc1cn[nH]c1 ; "S117" ; Clc1cc2c(s1)CCOC21CCN(Cc2cn[nH]c2)CC1 ; "1165"]

To a flask was added 2-chlorospiro[6,7-dihydrothieno[3,2-c]pyran-4,4′-piperidine](Trifluoroacetate salt) (400 mg, 1.1 mmol), 1H-pyrazole-4-carbaldehyde (105 mg, 1.1 mmol), acetic acid (70 μL, 1.231 mmol), and 1,2-dichloroethane (10 mL). The mixture was stirred at room temperature at which point triacetoxyboranuide (Sodium salt) (680 mg, 3.208 mmol) was added, and stirred overnight. The mixture was quenched with sat. sodium bicarbonate (25 mL), and the layers were separated and the aqueous layer was washed with DCM (50 mL). The organic layers were combined and concentrated to provide 2-chloro-1′-(1H-pyrazol-4-ylmethyl)spiro[6,7-dihydrothieno[3,2-c]pyran-4,4′-piperidine](360 mg, 94%) ¹H NMR (400 MHz, Chloroform-d) δ 7.57 (s, 2H), 6.62 (s, 1H), 3.93 (t, J=5.4 Hz, 2H), 3.54 (s, 2H), 2.81-2.75 (m, 2H), 2.72 (t, J=5.4 Hz, 2H), 2.37 (td, J=11.4, 4.0 Hz, 2H), 1.96-1.85 (m, 4H). ESI-MS m/z calc. 323.0859, found 324.26 (M+1)⁺.

### Compounds 1166-1168

Compounds 1166-1168 (see Table 55) were prepared from S117 using the appropriate aldehydes employing the same method as described for compound 1165.

TABLE 55
Structure and physicochemical data for compounds 1166-1168

| | | | LCMS |
|---|---|---|---|
| Cmpd | Product | Aldehyde | m/z [M + H]+ |
| 1166 | 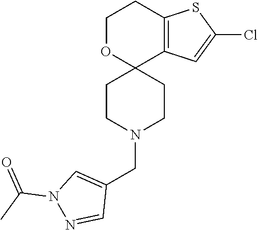 [SMILES: CC(=O)n1cc(CN2CCC3(CC2)OCCc2sc(Cl)cc23)cn1] | 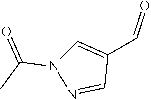 | ESI-MS m/z calc. 365.096, found 366.104 (M + 1)+ |
| 1167 | 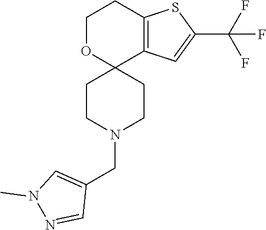 [SMILES: Cn1cc(CN2CCC3(CC2)OCCc2sc(C(F)(F)F)cc23)cn1] | 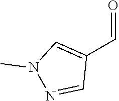 | ESI-MS m/z calc. 371.128, found 372.135 (M + 1)+ |
| 1168 | 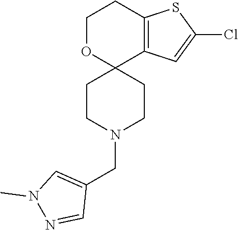 [SMILES: Cn1cc(CN2CCC3(CC2)OCCc2sc(Cl)cc23)cn1] | 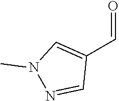 | ESI-MS m/z calc. 337.102, found 338.109 (M + 1)+ |

### Compound 1169

### 2′-chloro-1-((1-(2-(methylsulfonyl)ethyl)-1H-pyrazol-4-yl)methyl)-4′,5′-dihydrospiro[piperidine-4,7′-thieno[2,3-c]pyran] (1169)

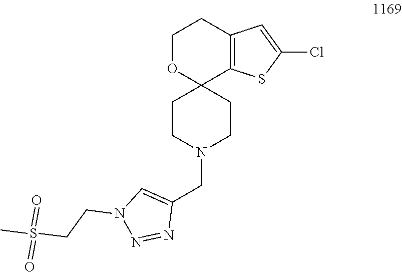 [SMILES: CS(=O)(=O)CCn1cc(CN2CCC3(CC2)OCCc2cc(Cl)sc23)nn1]

2′-chloro-1-((1-(2-(methylsulfonyl)ethyl)-1H-pyrazol-4-yl)methyl)-4′,5′-dihydrospiro[piperidine-4,7′-thieno[2,3-c]pyran] (1169) was prepared as described in the synthesis of Compound 697, with tert-butyl 4-oxopiperidine-1-carboxylate used in the place of tert-butyl (S)-2-methyl-4-oxopiperidine-1-carboxylate. The product was isolated as 2′-chloro-1-((1-(2-(methylsulfonyl)ethyl)-1H-pyrazol-4-yl)methyl)-4′,5′-dihydrospiro[piperidine-4,7′-thieno[2,3-c]pyran] (20.6 mg, 17%). ¹H NMR (400 MHz, Chloroform-d) δ 7.50 (d, J=0.7 Hz, 1H), 7.45 (s, 1H), 6.58 (s, 1H), 4.65-4.50 (m, 2H), 3.88 (t, J=5.4 Hz, 2H), 3.69-3.59 (m, 2H), 3.46 (s, 2H), 2.74-2.62 (m, 4H), 2.46 (d, J=0.8 Hz, 3H), 2.32 (td, J=10.9, 4.6 Hz, 2H), 1.84 (dd, J=11.0, 3.9 Hz, 4H). ESI-MS m/z calc. 429.98, found 430.13 (M+1)⁺.

### Compound 1170

### 2-ethyl-7-methyl-1′-[(1-methylpyrazol-4-yl)methyl]spiro[6,7-dihydrothieno[3,2-c]pyran-4,4′-piperidine](1170)

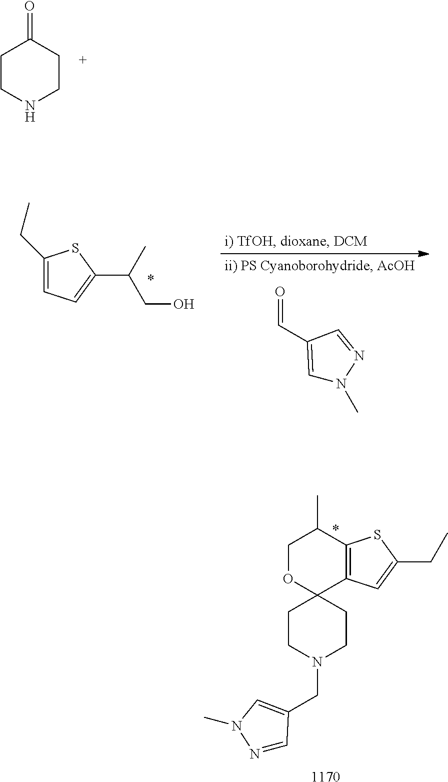

A solution of S105 2-(5-ethyl-2-thienyl)propan-1-ol and piperidin-4-one (51 mg, 0.30 mmol) and tert-butyl 4-oxopiperidine-1-carboxylate (50 mg, 0.25 mmol) in dioxane (1.2 mL) was cooled in an ice bath to just above freezing. To the solution was added trifluoromethanesulfonic acid (67 uL, 0.75 mmol), and the resulting solution stirred at room temperature for 1.5 hours. The reaction was quenched with water and adjusted to pH 8 with 2N Na₂CO₃. The solution was extracted with ethyl acetate and the combined organics dried over anhydrous sodium sulfate, filtered, and concentrated in vacuo.

To the crude reaction mixture was added 1-methylpyrazole-4-carbaldehyde (55 mg, 0.5 mmol), polymer supported (trimethylammonio)methyl (cyanoborohydride) (376 mg, 0.75 mmol, 2 mmol/g loading), and AcOH (71 uL, 1.25 mmol) in CH₂Cl₂ (2 mL). Reaction was irradiated in a microwave reactor at 95 C for 1 hour. Reaction was filtered, washed with MeOH, and concentrated in vacuo. The crude residue was purified via reverse phase HPLC (HCl modifier), to provide the desired product. 2-ethyl-7-methyl-1′-[(1-methylpyrazol-4-yl)methyl]spiro[6,7-dihydrothieno[3,2-c]pyran-4,4′-piperidine](40 mg, 41%). ESI-MS m/z calc. 345.187, found 346.195 (M+1)⁺.

### Synthesis of 2-ethyl-7-methyl-1′-[(1-methylpyrazol-4-yl)methyl]spiro[6,7-dihydrothieno[3,2-c]pyran-4,4′-piperidine](1171)

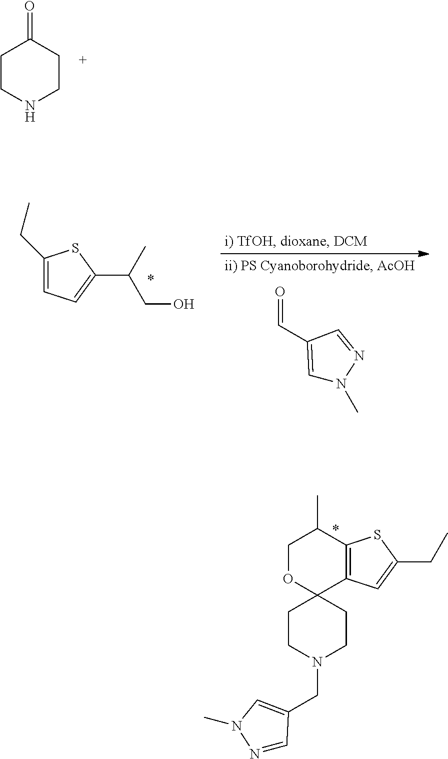

A solution of S106 2-(5-ethyl-2-thienyl)propan-1-oland piperidin-4-one (51 mg, 0.30 mmol) and tert-butyl 4-oxopiperidine-1-carboxylate (50 mg, 0.25 mmol) in dioxane (1.2 mL) was cooled in an ice bath to just above freezing. To the solution was added trifluoromethanesulfonic acid (67 uL, 0.75 mmol), and the resulting solution stirred at room temperature for 1.5 hours. The reaction was quenched with water and adjusted to pH 8 with 2N Na₂CO₃. The solution was extracted with ethyl acetate and the combined organics dried over anhydrous sodium sulfate, filtered, and concentrated in vacuo.

To the crude reaction mixture was added 1-methylpyrazole-4-carbaldehyde (55 mg, 0.5 mmol), polymer supported (trimethylammonio)methyl (cyanoborohydride) (376 mg, 0.75 mmol, 2 mmol/g loading), and AcOH (71 uL, 1.25 mmol) in CH₂Cl₂ (2 mL). Reaction was irradiated in a microwave reactor at 95 C for 1 hour. Reaction was filtered, washed with MeOH, and concentrated in vacuo. The crude residue was purified via reverse phase HPLC (HCl modifier), to provide the desired product. 2-ethyl-7-methyl-1′-[(1-methylpyrazol-4-yl)methyl]spiro[6,7-dihydrothieno[3,2-c]pyran-4,4′-piperidine](50 mg, 51%). ESI-MS m/z calc. 345.187, found 346.195 (M+1)⁺.

### Compounds 1172-1176

Compounds 1172-1176 (see Table 56) were prepared using the appropriate thiophene ethanol, piperidinone, and aldehyde employing the method described for compound 1171.

TABLE 56
Structure and physicochemical data for compounds 1172-1176

| | | | | | LCMS |
|---|---|---|---|---|---|
| | | | | | m/z |
| Cmpd | Product | Thiophene ethanol | Piperidinone | Aldehyde | [M + H]+ |
| 1170 | 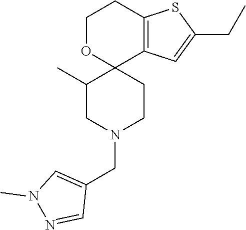 [SMILES: CCc1cc2c(s1)CCOC21CCN(Cc2cnn(C)c2)CC1C] | 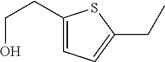 | 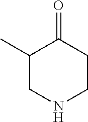 | 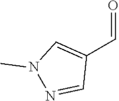 | ESI-MS m/z calc. 345.187, found 346.195 (M + 1)+ |
| 1171 | 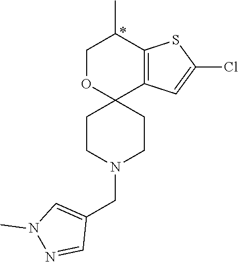 [SMILES: CC1COC2(CCN(Cc3cnn(C)c3)CC2)c2cc(Cl)sc21] | 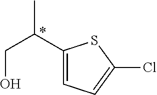 | 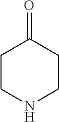 | 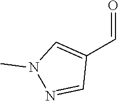 | ESI-MS m/z calc. 351.117, found 352.124 (M + 1)+ |
| 1172 | 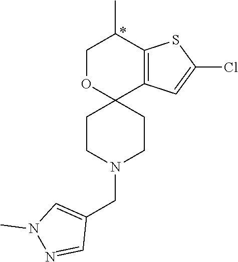 [SMILES: CC1COC2(CCN(Cc3cnn(C)c3)CC2)c2cc(Cl)sc21] | 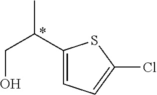 | 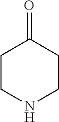 | 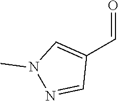 | ESI-MS m/z calc. 351.117, found 352.124 (M + 1)+ |
| 1173 | 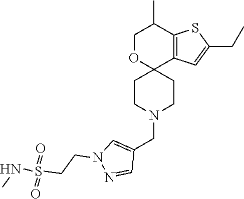 [SMILES: CCc1cc2c(s1)C(C)COC21CCN(Cc2cnn(CCS(=O)(=O)NC)c2)CC1] | 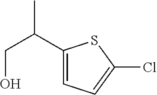 | 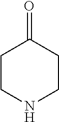 | 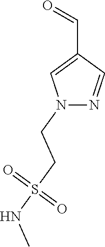 | ESI-MS m/z calc. 458.121, found 459.129 (M + 1)+ |
| 1174 | 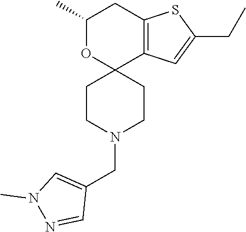 [SMILES: CCc1cc2c(s1)C[C@@H](C)OC21CCN(Cc2cnn(C)c2)CC1] | 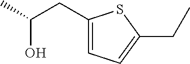 | 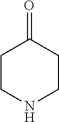 | 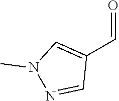 | ESI-MS m/z calc. 345.187, found 346.195 (M + 1)+ |

### Compound S118

### tert-butyl 2′-chloro-7′,7′-dimethyl-6′,7′-dihydrospiro[piperidine-4,4′-thieno[3,2-c]pyran]-1-carboxylate (S118)

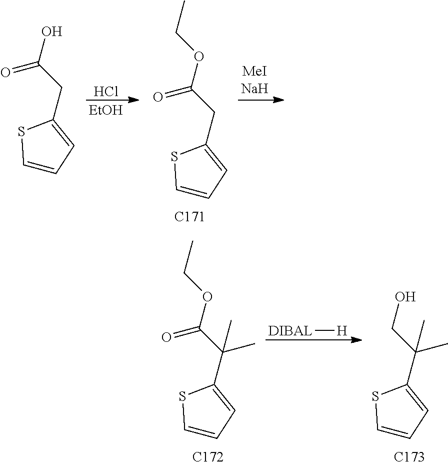

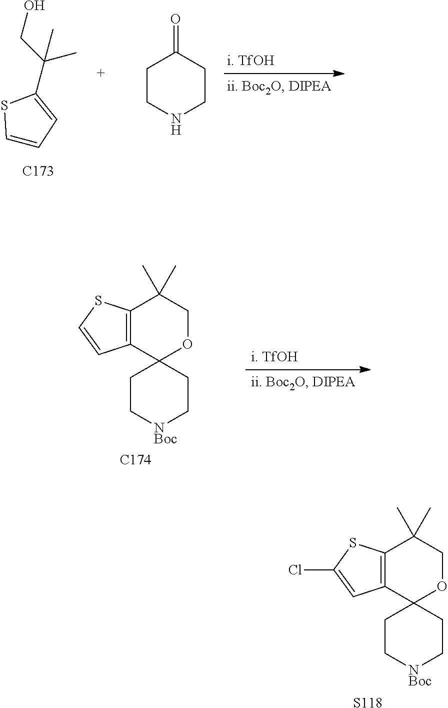

### Step 1. Synthesis of ethyl 2-(2-thienyl)acetate (C171)

To a stirred solution of 2-(2-thienyl)acetic acid (100 g, 703.35 mmol) in EtOH (2000 mL) was added HCl (Aqueous) (50 mL of 36% w/v, 493.68 mmol) at rt. The reaction mixture was stirred for 12 hrs at 70° C. After completion of the reaction, the mixture was concentrated and the resulting crude was diluted with EtOAc (1000 ml), washed with 5% Na₂CO₃ aqueous solution (3×200 ml), brine (200 ml), dried the organic layer and concentrated to afford ethyl 2-(2-thienyl)acetate (C171) (100 g, 82%) as a yellow oil. ¹H NMR (Chloroform-d, 400 MHz): δ=7.222-7.206 (dd, J=1.2 Hz, J=3.6 Hz, 1H), 6.969-6.948 (m, 2H), 4.213-4.160 (q, J=7.2 Hz, 2H), 3.828 (s, 2H), 1.295-1.259 (t, J=7.2 Hz, 3H). LCMS m/z 171.26 [M+H]⁺.

### Step 2. Synthesis of ethyl 2-methyl-2-(2-thienyl)propanoate (C172)

To a stirred solution of ethyl 2-(2-thienyl)acetate (C171) (100 mg, 531.87 mol) in THF (15 mL) was added NaH (108 mg, 60% w/w, 0.0027 mol) at 0° C., stirred for 30 min. Then to the reaction mixture MeI (440.00 mg, 0.2 mL, 0.0031 mol) was added at 0° C. and reaction was allowed to warm to rt, and then the reaction was stirred for 6 h. At this time, the reaction mixture was quenched with cold water (50 ml), extracted to EtOAC (2×100 ml). The combined organic layers were dried over Na₂SO₄, concentrated under pressure to afford ethyl 2-methyl-2-(2-thienyl)propanoate (C172) (40 mg, 36%) as a yellow liquid. ¹H NMR (400 MHz, Chloroform-d) δ ppm: 7.20-7.18 (m, 1H), 6.96-6.92 (m, 2H), 4.16-4.10 (m, 2H), 1.69-1.53 (m, 6H), 1.26-1.08 (m, 3H).

### Step 3. Synthesis of 2-methyl-2-(2-thienyl)propan-1-ol (C173)

To a stirred solution of ethyl 2-methyl-2-(2-thienyl)propanoate (C172) (500 mg, 0.0017 mol) in THF (10 mL) at rt. Then reaction mixture was cooled to 0° C., at which time DiBAL-H in hexane (5.10 mL of 1 M, 0.0051 mol) was added dropwise for 10 min. The reaction mixture was allowed to warm to rt and then stirred for 3 h. Then reaction mixture was cooled to 0° C., slowly quenched with sat. aq. NH₄Cl (100 mL), and then extracted with EtOAC (300 mL). The combined organic layers were dried over Na₂SO₄, concentrated under reduced pressure to get crude 2-methyl-2-(2-thienyl)propan-1-ol (C173) (272 mg, 99%) as a colorless liquid. ¹H NMR (DMSO-d₆): δ 7.31-7.29 (m, 1H), 6.94-6.87 (m, 2H), 4.89-4.84 (m, 1H), 3.39-3.36 (m, 2H), 1.35 (s, 6H).

### Step 4. Synthesis of tert-butyl 7,7-dimethylspiro[6H-thieno[3,2-c]pyran-4,4′-piperidine]-1′-carboxylate (C174)

To a solution of 2-methyl-2-(2-thienyl)propan-1-ol (C173) (250 mg, 1.600 mmol) and piperidin-4-one (Hydrochloride salt) (217 mg, 1.600 mmol) in dioxane (6 mL) and cooled in an ice bath was added trifluoromethanesulfonic acid (430 μL, 4.859 mmol). The reaction mixture was warmed to rt and stirred for 2 hrs. At this time, the mixture was diluted with 1N NaOH (80 mL) and EtOAc (80 mL) and the organic layer was dried and concentrated to a white solid which was resuspended in DCM (10 mL) and DIEA (500 μL, 2.871 mmol) and Boc₂O (400 mg, 1.833 mmol) was added and stirred at rt for 2 hrs. The reaction mixture was concentrated to an oil, which was purified by silica chromatography (0 to 80% EtOAc/heptane, 24 g silica) to yield tert-butyl 7,7-dimethylspiro[6H-thieno[3,2-c]pyran-4,4′-piperidine]-1′-carboxylate (C174) (401 mg, 74%) as a white solid. ¹H NMR (300 MHz, Chloroform-d) δ 7.11 (d, J=5.2 Hz, 1H), 6.69 (d, J=5.2 Hz, 1H), 4.11-3.82 (m, 2H), 3.59 (s, 2H), 3.27-3.04 (m, 2H), 1.88-1.72 (m, 4H), 1.51 (s, 9H), 1.32 (s, 6H).

### Step 5. Synthesis of tert-butyl 2-chloro-7,7-dimethyl-spiro[6H-thieno[3,2-c]pyran-4,4′-piperidine]-1′-carboxylate (S118)

To a solution of tert-butyl 7,7-dimethylspiro[6H-thieno[3,2-c]pyran-4,4′-piperidine]-1′-carboxylate (C174) (196 mg, 0.5808 mmol) in acetonitrile (5 mL) was added N-chlorosuccinimide (85 mg, 0.6365 mmol) and DMAP (7 mg, 0.05730 mmol). the reaction mixture was stirred at rt for 2 h, and then the reaction was heated to 65° C. and stirred for 18 h. At this time, the reaction mixture was concentrated to a solid which was diluted with 1N NaOH (20 mL) and EtOAc (20 mL). The organic layer washed with 1N aq. sodium thiosulfate solution and then dried with sodium sulfate, filtered and concentrated to a solid, which was purified by silica chromatography (0 to 50% EtOAc/heptane) to yield tert-butyl 2-chloro-7,7-dimethyl-spiro[6H-thieno[3,2-c]pyran-4,4′-piperidine]-1′-carboxylate (S118) (165 mg, 76%) as a white solid. ¹H NMR (300 MHz, Chloroform-d) δ 6.49 (s, 1H), 4.08-3.84 (m, 2H), 3.58 (s, 2H), 3.23-2.98 (m, 2H), 1.88-1.61 (m, 4H), 1.50 (s, 9H), 1.28 (s, 6H). LCMS m/z 371.29 [M+H]⁺.

### Compound 1177

### 2-[4-[(2-chloro-7,7-dimethyl-spiro[6H-thieno[3,2-c]pyran-4,4′-piperidine]-1′-yl)methyl]pyrazol-1-yl]-N-methyl-ethanesulfonamide (1177)

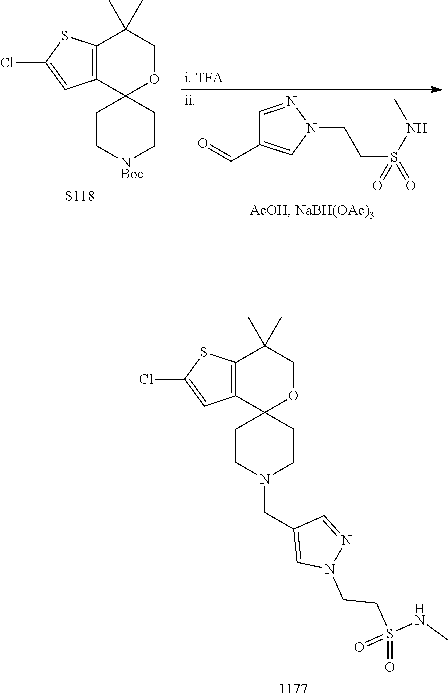

To a mixture of tert-butyl 2-chloro-7,7-dimethyl-spiro[6H-thieno[3,2-c]pyran-4,4′-piperidine]-1′-carboxylate (25 mg, 0.0672 mmol) (S118) and TFA (30 μL, 0.389 mmol) in dichloromethane (1 mL) was stirred at rt overnight. At this time, the mixtures were blown dry, diluted with DCM (5 mL), and blown dry once more. To the mixture was added 2-(4-formylpyrazol-1-yl)-N-methyl-ethanesulfonamide (17 mg, 0.0783 mmol), acetic acid (4 μL, 0.0703 mmol), and sodium triacetoxyboranuide (40 mg, 0.189 mmol) and 1,2-dichloroethane (1,500 μL) and stirred at rt. After 1 h, the mixture was heated to 50° C. and was stirred for 18 h. At this time, the mixture was heated to 65° C. and diluted with additional 1,2-dichlorethane (2 mL) and additional sodium triacetoxyboranuide (40 mg, 0.189 mmol) for 2 h. At this time, the mixtures were concentrated and then diluted in DMSO (1 mL). Purification by reversed-phase HPLC. Method: Waters XSelect CSH C18 OBD Prep Column; 30×150 mm, 5 micron. Gradient: Acetonitrile in Water with 0.1% Trifluoroacetic Acid. The product-containing fractions were concentrated to yield 2-[4-[(2-chloro-7,7-dimethyl-spiro[6H-thieno[3,2-c]pyran-4,4′-piperidine]-1′-yl)methyl]pyrazol-1-yl]-N-methyl-ethanesulfonamide (Trifluoroacetate salt) (1177) (13.3 mg, 33%) as a clear oil. ¹H NMR (400 MHz, Chloroform-d) δ 11.54 (s, 1H), 7.89 (s, 1H), 7.58 (s, 1H), 6.54 (s, 1H), 5.32 (s, 1H), 4.67-4.49 (m, 2H), 4.11 (d, J=4.3 Hz, 2H), 3.60-3.52 (m, 4H), 3.46 (d, J=11.8 Hz, 2H), 3.11 (q, J=11.1 Hz, 2H), 2.70 (s, 3H), 2.28 (td, J=14.2, 4.2 Hz, 2H), 2.06-1.98 (m, 2H), 1.28 (s, 6H). LCMS m/z 473.11 [M+H]⁺.

### Compound 1178

### 2-chloro-7,7-dimethyl-1′-[(1-methylpyrazol-4-yl)methyl]spiro[6H-thieno[3,2-c]pyran-4,4′-piperidine](1178)

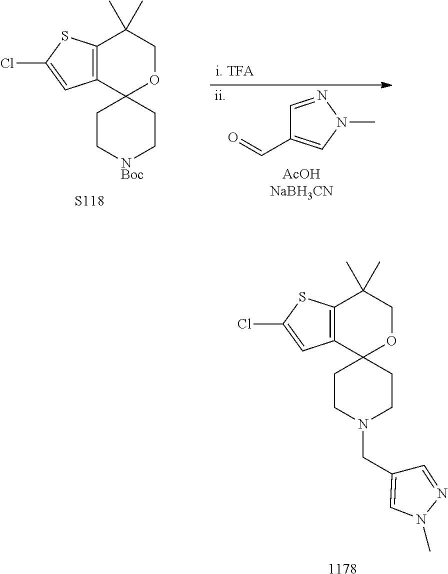

To a solution of tert-butyl 2-chloro-7,7-dimethyl-spiro[6H-thieno[3,2-c]pyran-4,4′-piperidine]-1′-carboxylate (S118) (60 mg, 0.1613 mmol) in DCM (1 mL) was added TFA (700 μL, 9.09 mmol). After 20 min., the reaction mixture was concentrated to an oil and redissolved in DCM (2 mL) and 1-methylpyrazole-4-carbaldehyde (35 mg, 0.318 mmol) was added followed by HOAc (50 μL, 0.8792 mmol) and polystyrene supported cyanoborohydride (200 mg of 2 mmol/g, 0.400 mmol). the reaction mixture was heated in a microwave for 60 min at 110° C., filtered off the resin and the filtrate was concentrated. The obtained oil was purified by reversed phase chromatography (C18, 5-100% MeCN:H₂O, 0.1 wt % TFA modifier). Procut-containing fractions were concentrated and diluted with EtOAc/1N NaOH. The organic layer was separated, dried with magnesium sulfate, filtered and concentrated to yield 2-chloro-7,7-dimethyl-1′-[(1-methylpyrazol-4-yl)methyl]spiro[6H-thieno[3,2-c]pyran-4,4′-piperidine](1178) (29 mg, 47%) as a clear oil. ¹H NMR (300 MHz, Chloroform-d) δ 7.43 (s, 1H), 7.32 (s, 1H), 6.55 (s, 1H), 3.90 (s, 3H), 3.55 (s, 2H), 3.48 (s, 2H), 2.82-2.65 (m, 2H), 2.45-2.25 (m, 2H), 1.94-1.79 (m, 4H), 1.27 (s, 6H). LCMS m/z 365.89 [M+H]⁺.

### Compound 1179

### 2′-chloro-1″-[(1-methyl-1H-pyrazol-4-yl)methyl]-6′H-dispiro[cyclopropane-1,7′-thieno[3,2-c]pyran-4′,4″-piperidine](1179)

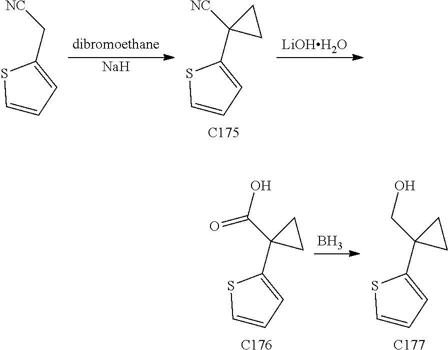

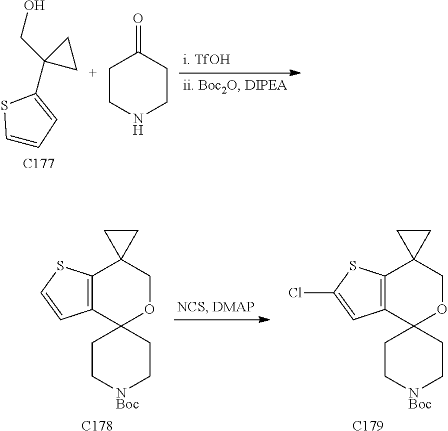

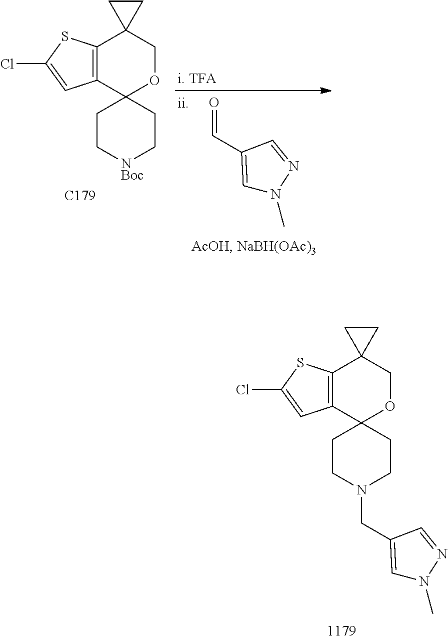

### Step 1. Synthesis of 1-(2-thienyl)cyclopropanecarbonitrile (C175)

To a stirred solution of 2-(2-thienyl)acetonitrile (2 g, 0.0162 mol) in DMF (30 mL) was added NaH (60% in mineral) (1.95 g, 60% w/w, 0.0488 mol) at 0° C. and stirred for 5 min. Then to the reaction mixture was added 1,2-dibromoethane (4.679 g, 2.2 mL, 0.0244 mol). The reaction was warmed to rt and stirred for 2 h. At this time, the reaction mixture was quenched with sat. aq. NH₄Cl and extracted with EtOAc (2×100 mL). The organic layer was washed with chilled brine sol, dried over Na₂SO₄ and concentrated under reduced pressure to get crude compound. It was purified by column chromatography (silica, 20% EtOAc:petroleum ether). The collected fractions were concentrated under reduced pressure to get 1-(2-thienyl)cyclopropanecarbonitrile (C175) (1.2 g, 47%) as a pale yellow oil. ¹H NMR (300 MHz, DMSO-d₆): δ 7.49 (dd, J=5.1 Hz, 1.2 Hz, 1H), 7.09 (dd, J=3.6 Hz, 1.2 Hz, 1H), 7.00 (t, J=3.6 Hz, 1H), 1.80 (t, J=5.1 Hz, 2H), 1.49 (t, J=4.8 Hz, 2H).

### Step 2. Synthesis of 1-(2-thienyl)cyclopropanecarboxylic acid (C176)

To a stirred solution of 1-(2-thienyl)cyclopropanecarbonitrile (C175) (4 g, 0.0263 mol) in Ethanol (15 mL) was added LiOH monohydrate (3.5 g, 0.0826 mol) in water (10 mL) at room temperature. The reaction mixture was stirred for 16 h at 100° C. Then reaction mixture was concentrated under reduced pressure. The crude mixture was dissolved in ice cold water (20 ml) and acidified with 1 N HCl (pH 3-4) to get solid. The solid was filtered and washed with excess water and dried on vacuum to afford 1-(2-thienyl)cyclopropanecarboxylic acid (C176) (3 g, 67%) as an off-white solid. ¹H-NMR (300 MHz, DMSO-d₆) δ 12.52 (s, 1H), 7.37-7.35 (m, 1H), 6.93-6.89 (m, 2H), 1.58-1.55 (m, 2H), 1.28-1.24 (m, 2H). LCMS m/z 169.24 [M+H]⁺.

### Step 3. Synthesis of [1-(2-thienyl)cyclopropyl]methanol (C177)

To a solution of 1-(2-thienyl)cyclopropanecarboxylic acid (C176) (1 g, 5.945 mmol) in THF (24 mL) and cooled to 0° C. was added borane-tetrahydrofuran (7.2 mL of 1 M, 7.200 mmol), dropwise. After 1 h, the reaction was quenched with H₂O and diluted with EtOAc (60 mL) and sat. aq. NH₄Cl 60 mL) The organic layer was washed with 1N NaOH (60 mL), brine (60 mL), dried with sodium sulfate and concentrated to an oil. The crude oil was purified by silica gel chromatography (0-60% EtOAc in Heptanes) to afford [1-(2-thienyl)cyclopropyl]methanol (C177) (840 mg, 85%) as a clear oil. ¹H NMR (300 MHz, Chloroform-d) δ 7.15 (dd, J=4.1, 2.3 Hz, 1H), 6.95-6.90 (m, 2H), 3.69 (d, J=6.2 Hz, 2H), 1.02-0.92 (m, 4H). LCMS m/z 154.94 [M+H]⁺.

### Step 4. Synthesis of tert-butyl 6′H-dispiro[cyclopropane-1,7′-thieno[3,2-c]pyran-4′,4″-piperidine]-1″-carboxylate (C178)

A solution of [1-(2-thienyl)cyclopropyl]methanol (C177) (235 mg, 1.524 mmol) and piperidin-4-one (Hydrochloride salt) (207 mg, 1.527 mmol) in dioxane (5 mL) was cooled in an ice bath. Trifluoromethanesulfonic acid (400 μL, 4.520 mmol) was added and the reaction mixture was warmed to rt and stirred for 2 h. The reaction mixture was diluted with 1N NaOH (60 mL) and EtOAc (60 mL) and the organic layer washed with brine, dried and concentrated to an oil which was diluted with DCM (10 mL) and DIPEA (500 μL, 2.871 mmol) and Boc₂O (330 mg, 1.512 mmol) added. After 1 h, the reaction mixture was concentrated and purified by silica gel chromatography (0 to 60% EtOAc/heptane) to give the product tert-butyl 6′H-dispiro[cyclopropane-1,7′-thieno[3,2-c]pyran-4′,4″-piperidine]-1″-carboxylate (C178) (333 mg, 65%)¹H NMR (400 MHz, Chloroform-d) δ 7.00 (d, J=5.2 Hz, 1H), 6.73 (d, J=5.2 Hz, 1H), 4.16-3.87 (m, 2H), 3.72 (s, 2H), 3.26-3.02 (m, 2H), 2.02-1.74 (m, 4H), 1.46 (s, 9H), 1.06-1.00 (m, 2H), 1.00-0.95 (m, 2H).

### Step 5. Synthesis of tert-butyl 2′-chloro-6′H-dispiro[cyclopropane-1,7′-thieno[3,2-c]pyran-4′,4″-piperidine]-1″-carboxylate (C179)

To a solution of tert-butyl 6′H-dispiro[cyclopropane-1,7′-thieno[3,2-c]pyran-4′,4″-piperidine]-1″-carboxylate (C178) (330 mg, 0.984 mmol) in acetonitrile (5 mL) was added NCS (150 mg, 1.123 mmol) and DMAP (10 mg, 0.08185 mmol). The reaction mixture was heated to 50° C. for 16 h, and then cooled to rt and stirred for 2 days. At this time, the mixture was diluted with 1N NaOH (50 mL) and EtOAc (50 mL) and the organic layer washed with sodium thiosulfate solution (50 mL), brine (50 mL), dried over Na₂SO₄ and concentrated to an oil, which was purified by silica gel chromatography (0 to 50% EtOAc/heptane) to yield tert-butyl 2′-chloro-6′H-dispiro[cyclopropane-1,7′-thieno[3,2-c]pyran-4′,4″-piperidine]-1″-carboxylate (C179) (269 mg, 74%) as a tacky white solid. ¹H NMR (300 MHz, Chloroform-d) δ 6.54 (s, 1H), 4.09-3.88 (m, 2H), 3.70 (s, 2H), 3.12 (t, J=12.8 Hz, 2H), 1.99-1.85 (m, 2H), 1.76 (td, J=13.4, 4.8 Hz, 2H), 1.50 (s, 9H), 1.04-0.95 (m, 2H), 0.95-0.84 (m, 2H).

### Step 6. Synthesis of 2′-chloro-1″-[(1-methyl-1H-pyrazol-4-yl)methyl]-6′H-dispiro[cyclopropane-1,7′-thieno[3,2-c]pyran-4′,4″-piperidine] (1179)

To a solution of tert-butyl 2′-chloro-6′H-dispiro[cyclopropane-1,7′-thieno[3,2-c]pyran-4′,4″-piperidine]-1″-carboxylate (C179) (67 mg, 0.181 mmol) n DCM (1 mL) was added TFA (500 μL, 6.49 mmol). After 20 min, the solvent was evaporated and the crude mixture was dissolved in DCE (1.5 mL) and 1-methylpyrazole-4-carbaldehyde (35 mg, 0.318 mmol) was added followed by AcOH (52 μL, 0.914 mmol) and triacetoxyboranuide (Sodium salt) (60 mg, 0.283 mmol) and the reaction mixture heated to 60° C. After 2 h, additional triacetoxyboranuide (Sodium salt) (50 mg, 0.236 mmol) was added and the reaction mixture heated to 60° C. and stirred 18 h. At this time, the reaction mixture was diluted with 1N NaOH (20 mL) and EtOAc (20 mL) and the organic layer dried with Na₂SO₄ and concentrated to an oil which was purified by C18 column chromatography (10-100% MeCN:water, 0.1 wt % TFA modifier). The pure fractions were treated with 1N NaOH (5 mL) and extracted with EtOAc (50 mL). The organic layer was concentrated to yield 2′-chloro-1″-[(1-methyl-1H-pyrazol-4-yl)methyl]-6′H-dispiro[cyclopropane-1,7′-thieno[3,2-c]pyran-4′,4″-piperidine](1179) (16 mg, 23%) as a yellow oil. ¹H NMR (300 MHz, Chloroform-d) δ 7.43 (s, 1H), 7.32 (s, 1H), 6.59 (s, 1H), 3.90 (s, 3H), 3.67 (s, 2H), 3.48 (s, 2H), 2.85-2.66 (m, 2H), 2.45-2.25 (m, 2H), 1.97-1.87 (m, 4H), 1.02-0.93 (m, 2H), 0.93-0.84 (m, 2H). LCMS m/z 364.36 [M+H]⁺.

### Compound 1180

### 2-chloro-4-isopropyl-1′-[[1-(2-methylsulfonylethyl)triazol-4-yl]methyl]spiro[4,5-dihydrothieno[2,3-c]pyran-7,4′-piperidine

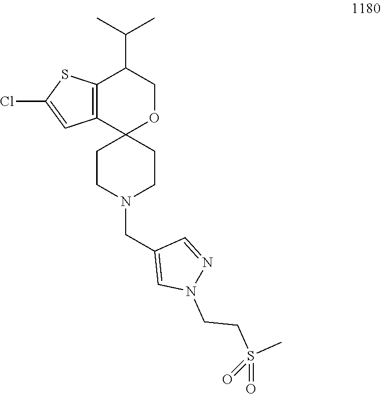 [SMILES: CC(C)C1COC2(CCN(Cc3cnn(CCS(C)(=O)=O)c3)CC2)c2cc(Cl)sc21]

Compound (1180) was synthesized according to a similar chemical strategy used to synthesize compound 1177. 2-chloro-4-isopropyl-1′-[[1-(2-methylsulfonylethyl)triazol-4-yl]methyl]spiro[4,5-dihydrothieno[2,3-c]pyran-7,4′-piperidine](1180) (4.3 mg, 18%) was isolated as a white solid. ¹H NMR (300 MHz, Methanol-d₄) δ 8.03 (s, 1H), 6.72 (s, 1H), 4.92 (t, J=6.6 Hz, 2H), 3.91-3.77 (m, 4H), 3.75 (s, 2H), 2.95-2.86 (m, 4H), 2.82-2.66 (m, 2H), 2.57 (dt, J=7.4, 3.6 Hz, 1H), 2.54-2.39 (m, 2H), 2.05-1.66 (m, 5H), 0.99 (dd, J=13.9, 6.8 Hz, 6H). ESI-MS m/z calc. 472.137, found 473.145 (M+1)⁺.

### Preparation of 4-(chloromethyl)-1-(2-methylsulfonylethyl)triazole (S119)

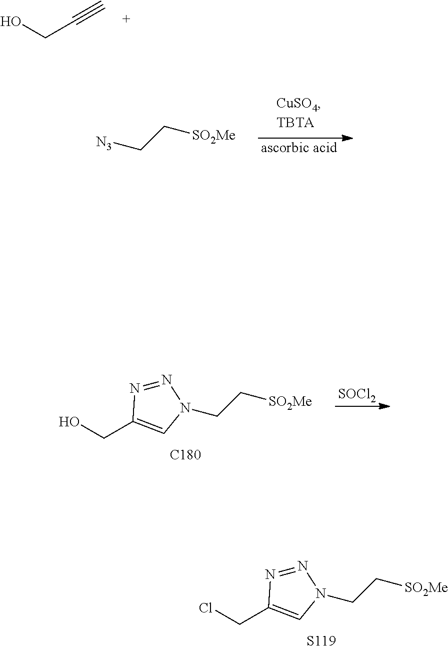

### Step 1. Synthesis of [1-(2-methylsulfonylethyl)triazol-4-yl]methanol (C180)

A mixture of prop-2-yn-1-ol (500 μL, 8.464 mmol), 1-azido-2-methylsulfonyl-ethane (1200 mg, 8.044 mmol), CuSO₄ (16 mg, 0.100 mmol), 1-(1-benzyltriazol-4-yl)-N,N-bis[(1-benzyltriazol-4-yl)methyl]methanamine (TBTA) (100 mg, 0.189 mmol), (2R)-2-[(1S)-1,2-dihydroxyethyl]-3,4-dihydroxy-2H-furan-5-one (1500 mg, 8.517 mmol) in MeOH (40 mL)/water (10 mL) was heated to 50° C. (11:00). After 3 h, TLC indicated complete consumption of the azide. The reaction was cooled to rt and concentrated. The mixture was diluted until full dissolution in MeOH, and then silica was added and the mixture was concentrated down to a dry solid. The solid was loaded onto a SiO2 column for purification (0-10% MeOH:DCM) to yield [1-(2-methylsulfonylethyl)triazol-4-yl]methanol (C180) (1210 mg, 70%) as a yellow solid. ¹H NMR (400 MHz, DMSO-d₆) δ 8.04 (s, 1H), 5.21 (d, J=6.8 Hz, 1H), 4.81 (t, J=7.0 Hz, 2H), 4.54 (d, J=4.5 Hz, 2H), 3.80 (t, J=6.9 Hz, 2H), 2.96 (s, 3H). LCMS m/z 206.4 [M+H]⁺.

### Step 2. Synthesis of 4-(chloromethyl)-1-(2-methylsulfonylethyl)triazole (S119)

The solid from the first step was diluted with DCE (30 mL) followed by DMF (5 mL), and to this mixture heated to 50° C. was added thionyl chloride (550 μL, 7.540 mmol) which immediately resulted in a clear yellow solution. The mixture was stirred at this temperature (1:30). After stirring 30 min, the mixture was concentrated to a minimal volume and diluted in diethyl ether (20 mL) to precipitate a yellow solid which was filtered and rinsed with additional ether to yield 4-(chloromethyl)-1-(2-methylsulfonylethyl)triazole (S119) (928 mg, 74%) as a yellow solid. ¹H NMR (400 MHz, Chloroform-d) δ 7.80 (s, 1H), 4.92-4.86 (m, 2H), 4.74 (d, J=0.6 Hz, 2H), 3.74 (ddd, J=7.1, 5.6, 0.7 Hz, 2H), 2.77 (t, J=0.7 Hz, 3H). LCMS m/z 224.36 [M+H]⁺.

### Compound 1181

### 2-chloro-1′-[[1-(2-methylsulfonylethyl)pyrazol-4-yl]methyl]spiro[4,5-dihydrothieno[2,3-c]pyran-7,4′-piperidine]-4-ol (1181)

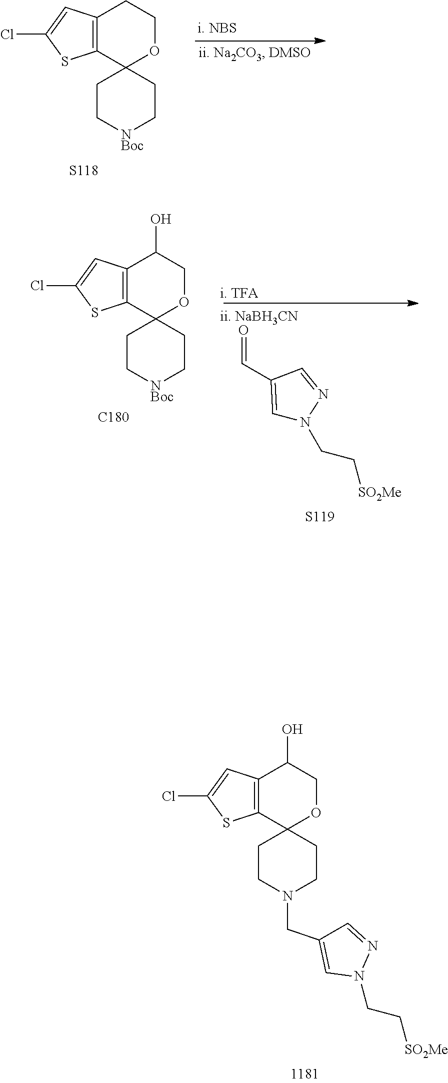

### Step 1. Synthesis of tert-butyl 2-chloro-4-hydroxy-spiro[4,5-dihydrothieno[2,3-c]pyran-7,4′-piperidine]-1′-carboxylate (C180)

A mixture of tert-butyl 2-chlorospiro[4,5-dihydrothieno[2,3-c]pyran-7,4′-piperidine]-1′-carboxylate (S118) (358 mg, 0.8329 mmol) in ethyl acetate (10 mL) was stirred and was purged and sonicated under argon for 15 minutes. At this time, 1-bromopyrrolidine-2,5-dione (175 mg, 0.9832 mmol) in ethyl acetate (5 mL) was added and the mixture was stirred to dissolution and immediately passed through a commercial photoreactor (450 nm Blue LED, 4 min residence time, 30° C., 2 mL photoreactor, 0.5 mL/min pump rate). The mixture was mixed with sodium bisulfite (10 mL) to consume excess NBS, separated, and the aqueous layer was extracted with ethyl acetate (10 mL) and the combined organic layer was dried with sodium sulfate, filtered, and concentrated. To the crude mixture was added sodium bicarbonate (42 mg, 0.500 mmol) and DMSO (1 mL), and the mixture was stirred at 50° C. for 3 h. At this time, the mixture was cooled to rt, diluted with EtOAc (20 mL) and water (5 mL). The layers were separated and concentrated to a minimum volume and was loaded onto a C18 column and purified (40-100% MeCN:water, 0.1 wt % TFA modifier). The product-containing fractions were pooled and concentrated to yield tert-butyl 2-chloro-4-hydroxy-spiro[4,5-dihydrothieno[2,3-c]pyran-7,4′-piperidine]-1′-carboxylate (C180) (18 mg, 15%) as a white solid. ¹H NMR (400 MHz, Chloroform-d) δ 6.77 (s, 1H), 4.90-4.63 (m, 1H), 4.39 (t, J=2.9 Hz, 1H), 4.00-3.78 (m, 4H), 3.07 (dt, J=45.4, 12.4 Hz, 2H), 2.06-1.90 (m, 1H), 1.90-1.78 (m, 1H), 1.70 (td, J=13.1, 4.9 Hz, 1H), 1.53 (ddd, J=14.2, 12.7, 4.8 Hz, 1H), 1.41 (s, 9H). LCMS m/z 360.48 [M+H]⁺.

### Step 2. Synthesis of 2-chloro-1′-[[1-(2-methylsulfonylethyl)pyrazol-4-yl]methyl]spiro[4,5-dihydrothieno[2,3-c]pyran-7,4′-piperidine]-4-ol (1181)

To a mixture of tert-butyl 2-chloro-4-hydroxy-spiro[4,5-dihydrothieno[2,3-c]pyran-7,4′-piperidine]-1′-carboxylate (C180) (18 mg, 0.0400 mmol) in dichloromethane (500 μL) was added hydrogen chloride (100 μL of 4 M, 0.400 mmol) in dioxane. At this time, diethyl ether was added (2×3 mL) to precipitate the product and decanted off to remove dioxane. The mixture was dried and redissolved in dichloromethane (2,000 μL) and transferred to a 5 mL microwave vial. Polystyrene supported cyanoborohydride (100 mg of 2 mmol/g, 0.200 mmol), 1-(2-methylsulfonylethyl)pyrazole-4-carbaldehyde (S119) (10.34 mg, 0.0440 mmol) and acetic acid (10 μL, 0.176 mmol) were added and the mixture was capped, sealed, and heated to 110° C. for 20 minutes. At this time, UPLC indicated complete conversion and the mixture was filtered, concentrated, redissolved minimally in DCM and loaded onto a SiO2 column (0-10% MeOH:DCM). The product-containing fractions were pooled and concentrated to yield 2-chloro-1′-[[1-(2-methylsulfonylethyl)pyrazol-4-yl]methyl]spiro[4,5-dihydrothieno[2,3-c]pyran-7,4′-piperidine]-4-ol (1181) (7 mg, 38%) as a white solid. ¹H NMR (400 MHz, Chloroform-d) δ 7.51 (s, 1H), 7.46 (s, 1H), 6.82 (s, 1H), 4.63-4.54 (m, 2H), 4.41 (t, J=2.8 Hz, 1H), 3.94 (dd, J=12.4, 2.9 Hz, 1H), 3.86 (dd, J=12.3, 2.8 Hz, 1H), 3.64 (t, J=6.1 Hz, 2H), 3.48 (s, 2H), 2.71 (d, J=15.7 Hz, 2H), 2.49 (s, 4H), 2.32 (t, J=12.0 Hz, 1H), 1.96 (s, 2H), 1.76 (d, J=12.0 Hz, 1H), 0.85 (d, J=18.7 Hz, 2H). LCMS m/z 446.07 [M+H]⁺.

### Compound 1182

### 2,7-diethyl-1′-[[1-(2-methylsulfonylethyl)triazol-4-yl]methyl]spiro[6,7-dihydrothieno[3,2-c]pyran-4,4′-piperidine](1182)

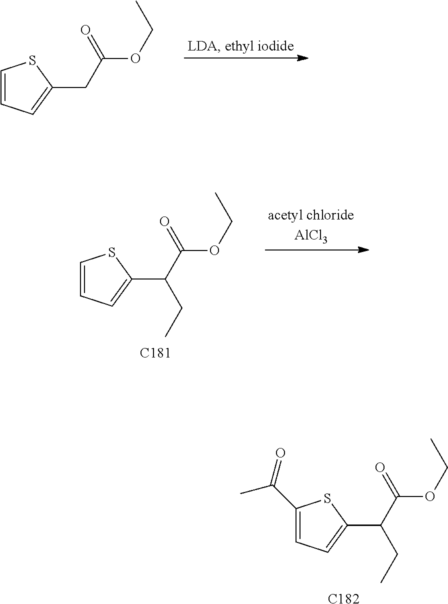 [SMILES: CCOC(=O)Cc1cccs1 ; CCOC(=O)C(CC)c1cccs1 ; "C181" ; CCOC(=O)C(CC)c1ccc(C(C)=O)s1 ; "C182"]

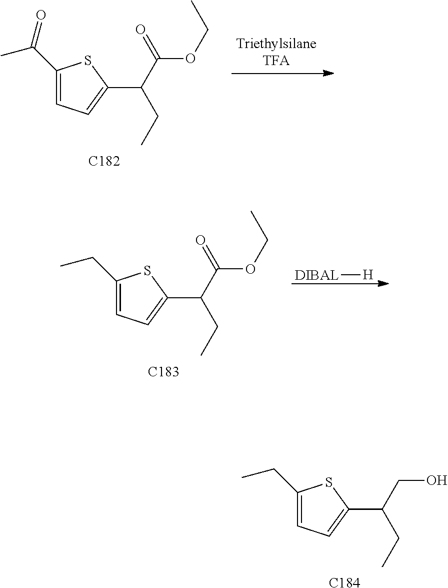

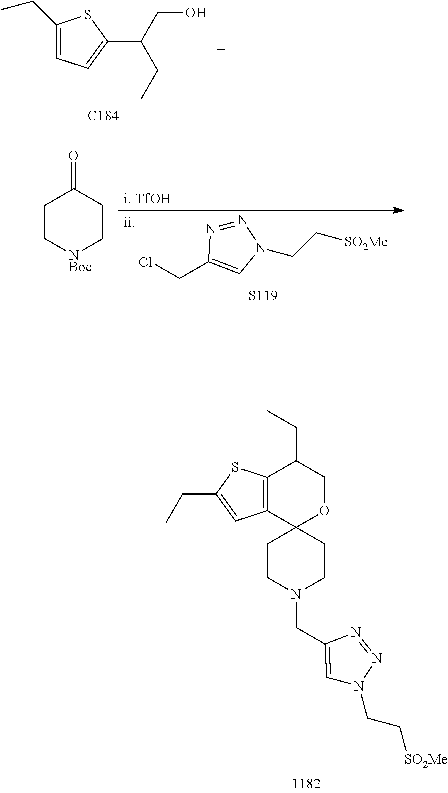 [SMILES: CCc1ccc(C(CC)CO)s1 ; "C184" ; CC(C)(C)OC(=O)N1CCC(=O)CC1 ; CS(=O)(=O)CCn1cc(CCl)nn1 ; "S119" ; CCc1cc2c(s1)C(CC)COC21CCN(Cc2cn(CCS(C)(=O)=O)nn2)CC1]

### Step 1. Synthesis of ethyl 2-(2-thienyl)butanoate (C181)

To a stirred solution of ethyl 2-(2-thienyl)acetate (441 μL, 2.94 mmol) in THF (10 mL) was added LDA (2.9 mL of 2 M, 5.800 mmol) dropwise at −40° C. The reaction mixture was stirred for 1 hour at −40° C. and then iodoethane (280 μL, 3.501 mmol) was added dropwise. The reaction as stirred at −40° C. for 2 h and then 30 min at rt. At this time, the mixture was slowly quenched with sat. aq. NH₄Cl (1000 ml) and extracted with diethyl ether (3×500 ml). The total organic layer was washed with brine (500 ml), dried the organic layer over sodium sulfate and concentrated to afford crude product, the crude product was further purified by normal phase column chromatography (0-30% ethyl acetate in heptane). The isolated product was a mixture of ethyl 2-(2-thienyl)butanoate (C181) (370 mg, 23%) as a yellow oil. ¹H NMR (300 MHz, Chloroform-d) δ 7.13-7.00 (m, 1H), 6.85 (q, J=2.2 Hz, 2H), 4.17-3.93 (m, 2H), 3.69-3.56 (m, 1H), 2.15-1.87 (m, 2H), 1.78 (dp, J=13.5, 7.3 Hz, 1H), 1.29-1.05 (m, 3H), 0.86 (t, J=7.4 Hz, 2H), 0.72 (t, J=7.4 Hz, 1H). LCMS m/z 227.1 [M+H]⁺.

### Step 2. Synthesis of ethyl 2-(5-acetyl-2-thienyl)butanoate (C182)

To a stirred solution of ethyl 2-(2-thienyl)butanoate (C181) (360 mg, 1.089 mmol) in DCM (3 mL) was added acetyl chloride (128 mg, 1.631 mmol) dropwise at 0° C., after that AlCl3 (218 mg, 1.635 mmol) was added portion wise at 0° C. The reaction mixture was stirred at 0° C. for 2 h. At this time, the mixture was slowly quenched with ice water (20 mL) and extracted with DCM (20 mL). The total organic layer was washed with brine (20 mL), dried over sodium sulfate and concentrated to afford crude compound, which was further purified by silica gel column chromatography (4 g 0-20% ethyl acetate in heptane). The pure fractions were combined and concentrated to afford ethyl 2-(5-acetyl-2-thienyl)butanoate (C182) (130 mg, 83%) as a yellow oil. ¹H NMR (300 MHz, Chloroform-d) δ 7.56 (d, J=3.9 Hz, 1H), 6.99 (dd, J=3.9, 0.6 Hz, 1H), 4.19 (m, 2H), 3.76 (t, J=7.6 Hz, 1H), 2.53 (s, 3H), 2.26-2.03 (m, 1H), 2.02-1.81 (m, 1H), 1.27 (t, J=7.1 Hz, 3H), 0.96 (t, J=7.4 Hz, 3H).

### Step 3. Synthesis of ethyl 2-(5-ethyl-2-thienyl)butanoate (C183)

To a stirred solution of ethyl 2-(5-acetyl-2-thienyl)butanoate (C182) (130 mg, 0.541 mmol) in TFA (780 μL) was added, dropwise, triethyl silane (130 μL, 0.814 mmol) at 0° C. The solution was warmed to rt. After 4 h, another equivalent of triethyl silane (130 μL, 0.814 mmol) was added. After 1 h, the mixture was concentrated and quenched with ice water (20 mL) and extracted with EtOAc (20 mL). The total organic were washed with brine (20 mL), dried over sodium sulfate and concentrated to afford crude product. The crude product was further purified by silica gel column chromatography (0-20% ethyl acetate in heptane) to afford ethyl 2-(5-ethyl-2-thienyl)butanoate (C183) (74 mg, 60%) as a yellow oil. LCMS m/z 227.1 [M+H]⁺.

### Step 4. Synthesis of 2-(5-ethyl-2-thienyl)butan-1-ol (C184)

To a stirred solution of ethyl 2-(5-ethyl-2-thienyl)butanoate (C183) (74 mg, 0.3237 mmol) in THF (1.46 mL) was added DIBAL-H (809 μL of 1 M, 0.809 mmol) dropwise at 0° C. The reaction was stirred for 4 h. At this time, the reaction was quenched with sat. aq. NH₄Cl (5 mL) and extracted with EtOAc (5 mL). The organics were then washed with brine (5 mL), dried over sodium sulfate and concentrated to give 2-(5-ethyl-2-thienyl)butan-1-ol (C184) (49 mg, 81%) as a yellow oil. ¹H NMR (300 MHz, Chloroform-d) δ 6.66-6.36 (m, 2H), 3.78-3.34 (m, 2H), 3.00-2.56 (m, 3H), 1.86-1.32 (m, 3H), 1.22 (t, J=7.5 Hz, 3H), 0.84 (t, J=7.4 Hz, 3H). LCMS m/z 185.08 [M+H]⁺.

### Step 5. Synthesis of 2,7-diethyl-1′-[[1-(2-methylsulfonylethyl)triazol-4-yl]methyl]spiro[6,7-dihydrothieno[3,2-c]pyran-4,4′-piperidine] (1182)

To a solution of 2-(5-ethyl-2-thienyl)butan-1-ol (C184) (49 mg, 0.266 mmol) and tert-butyl 4-oxopiperidine-1-carboxylate (48 mg, 0.241 mmol) in dioxane (960 μL) cooled to 0° C. was added trifluoromethanesulfonic acid (111 mg, 0.740 mmol), dropwise. The ice bath was removed, and the reaction was stirred for 3 h. The reaction was quenched with sat. aq. sodium bicarbonate solution then diluted with EtOAc (5 mL) and the aqueous layer was extracted with additional EtOAc (3×5 mL). The combined organic layer was evaporated to dryness.

To the crude reaction mixture was added 4-(chloromethyl)-1-(2-methylsulfonylethyl)triazole (S119) (110 mg, 0.492 mmol) were dissolved in DCE (2.6 mL). DIPEA (128 μL, 0.735 mmol) was added dropwise. The resulting solution was stirred at rt for 1 h and then it was heated to 65° C. and then stirred for 18 h. After 24 hours the solution as cooled and partitioned between DCM (10 mL) and 1N NaOH (10 mL). The organic layer was collected through a phase separator tube then concentrated in vacuo. The product was purified by silica gel chromatography (0-20 Ethyl Acetate:heptane) to yield 2,7-diethyl-1′-[[1-(2-methylsulfonylethyl)triazol-4-yl]methyl]spiro[6,7-dihydrothieno[3,2-c]pyran-4,4′-piperidine](Trifluoroacetate salt) (1182) (15.5 mg, 11%) as a yellow oil. ¹H NMR (300 MHz, Methanol-d₄) δ 8.30 (s, 1H), 6.48 (s, 1H), 4.99 (t, J=6.4 Hz, 2H), 4.51 (s, 2H), 3.95 (dd, J=11.6, 4.1 Hz, 1H), 3.83 (t, J=6.3 Hz, 2H), 3.70 (dd, J=11.6, 5.2 Hz, 1H), 3.50-3.35 (m, 3H), 3.00 (s, 3H), 2.77 (q, J=7.5 Hz, 3H), 2.10 (dd, J=14.0, 8.6 Hz, 4H), 1.82-1.48 (m, 2H), 1.25 (t, J=7.4 Hz, 3H), 1.02 (t, J=7.4 Hz, 3H). ESI-MS m/z calc. 452.192, found 453.199 (M+1)⁺.

### Example 2. Assays for Detecting and Measuring APOL1 Inhibitor Properties of Compounds

### MultiTox-Fluor Multiplex Cytotoxicity Assay

The MultiTox-Fluor Multiplex Cytotoxicity Assay is a single-reagent-addition, homogeneous, fluorescence assay that measures the number of live and dead cells simultaneously in culture wells. The assay measures cell viability and cytotoxicity by detecting two distinct protease activities. The live-cell protease activity is restricted to intact viable cells and is measured using a fluorogenic, cell-permeant peptide glycyl-phenylalanylamino fluorocoumarin (GF-AFC) substrate. The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R 110 Substrate) is used to measure dead-cell protease that has been released from cells that have lost membrane integrity. A ratio of dead to live cells is used to normalize data.

Briefly, the tet-inducible transgenic APOL1 T-REx-HEK293 cell lines were incubated with 50 ng/mL tet to induce APOL1 in the presence of 3-(2-(4-fluorophenyl)-1H-indol-3-yl)-N-((3S,4R)-4-hydroxy-2-oxopyrrolidin-3-yl)propenamide at 10.03, 3.24, 1.13, 0.356, 0.129, 0.042, 0.129, 0.0045, 0.0015, 0.0005 μM in duplicate for 24 hours in a humidified 37° C. incubator. The MultiTox reagent was added to each well and placed back in the incubator for an additional 30 minutes. The plate was read on the EnVision plate reader. A ratio of dead to live cells was used to normalize, and data was imported, analyzed, and fit using Genedata Screener (Basel, Switzerland) software. Data was normalized using percent of control, no tet (100% viability), and 50 ng/mL tet treated (0% viability), and fit using Smart Fit. The reagents, methods, and complete protocol for the MultiTox assay are described below.

TABLE 49
Reagents Used in the Multi-Tox Assay

| Reagent | Catalog Number | Vendor |
|---|---|---|
| 384 well, transparent, flat | 356663 | Corning (Corning, NY) |
| bottom tissue culture | | |
| treated, Poly-D lysine | | |
| coated | | |
| 384 well round bottom | 3656 | CoStar (Corning, NY) |
| polypropylene plates | | |
| Universal plate lids | 250002 | Thermo Fisher |
| | | (Waltham) |
| Axygen 30 μL tips for | VT-384-31UL-R-S | Corning (Corning, NY) |
| Bravo 384 well | | |
| MultiTox-Fluor Multiplex | G9202 | Promega (Madison, WI) |
| Cytotoxicity Assay | | |
| 225 cm² flask, angled neck, | 431082 | Corning (Corning, NY) |
| treated, vented cap | | |
| Dulbecco's Phosphate- | 14190-136 | Thermo Fisher |
| Buffered Saline (DPBS), | | (Waltham) |
| calcium and magnesium- | | |
| free | | |
| Dulbecco's Modified Eagle | 11960-077 | Thermo Fisher |
| Medium (DMEM), high | | (Waltham) |
| glucose, no glutamine, no | | |
| sodium pyruvate | | |
| Fetal Bovine Serum (FBS), | 631368 | Takara (Kusatsu, Japan) |
| tetracycline-free, US- | | |
| Sourced | | |
| L-Glutamine, 200 mM | 25030-081 | Thermo Fisher |
| | | (Waltham) |
| Penicillin-Streptomycin, | 15140-122 | Thermo Fisher |
| 10,000 Units/mL | | (Waltham) |
| Blasticidin S HCl, 10 | A11139-03 | Thermo Fisher |
| mg/mL | | (Waltham) |
| Tetracycline hydrochloride | T7660 -5G | Sigma (St. Louis, MO) |
| Puromycin dihydrochloride, | A11138-03 | Thermo Fisher |
| 10 mg/mL | | (Waltham) |
| Trypsin-EDTA | 25300-054 | Thermo Fisher |
| | | (Waltham) |

TABLE 50
Equipment Used in the Multi-Tox Assay

| Instrument | Model | Supplier | Location |
|---|---|---|---|
| Bravo | 16050-101 | Agilent | Santa Clara, CA |
| | | Technologies | |
| Multidrop Combi | N/A | Thermo | Waltham, MA |
| | | Scientific | |
| En Vision | N/A | PerkinElmer | Waltham, MA |

### Multi-Tox Assay Protocol

Human embryonic kidney (HEK293) cell lines containing a tet-inducible expression system (T-REx™; Invitrogen, Carlsbad, CA) and Adeno-associated virus site 1 pAAVS1-Puro-APOL1 G0 or pAAVS1-Puro-APOL1 G1 or pAAVS1-Puro-APOL1 G2 Clones G0 DC2.13, G1 DC3.25, and G2 DC4.44 were grown in a T-225 flask at ˜90% confluency in cell growth media (DMEM, 10% Tet-free FBS, 2 mM L-glutamine, 100 Units/mL penicillin-streptomycin, 5 μg/mL blasticidin S HCl, 1 μg/mL puromycin dihydrochloride). Cells were washed with DPBS and then trypsinized to dissociate from the flask. Media was used to quench the trypsin, cells were then pelleted at 200 g and resuspended in fresh cell assay media (DMEM, 2% Tet-free FBS, 2 mM L-glutamine, 100 Units/mL penicillin-streptomycin). Cells were counted and diluted to 1.17×10⁶ cells/mL. 20 μL of cells (23,400/well) were dispensed in every well of a 384-well Poly-D-Lysine coated plate using the Multidrop dispenser. The plates were then incubated at room temperature for one hour.

Tetracycline is needed to induce APOL1 expression. 1 mg/mL tet stock in water was diluted to 250 ng/mL (5×) in cell assay media. 60 μL of cell assay media (no tet control) was dispensed in columns 1 and 24, and 60 μL of 5× tet in 384-PP-round bottom plate was dispensed in columns 2 to 23 with the Multidrop dispenser.

Assay ready plates from the Global Compound Archive were ordered using template 384_APOL1Cell_DR10n2_50 uM_v3. Compounds were dispensed at 200 nL in DMSO. The final top concentration was 10 μM with a 10 point 3-fold dilution in duplicate in the MultiTox assay.

20 μL was transferred from the 5× tet plate to the ARP and mixed, then 5 μL of 5× tet and the compounds were transferred to the cell plate and mixed using the Bravo. The cell plate was placed in the humidified 37° C. 5% CO₂ incubator for 24 hours.

The MultiTox-Fluor Multiplex Cytotoxicity Assay was performed in accordance with the manufacturer's protocol. After cells were incubated with tet and compound for 24 hours, 25 μL of 1× MultiTox reagent was added to each well using the Multidrop dispenser; the plates were placed on a plate shaker (600 rpm) for 2 minutes, then centrifuged briefly and placed back in the 37° C. incubator for 30 minutes. The cell viability (excitation: 400 nm, emission: 486 nm) and cytotoxicity (excitation: 485 nm, emission: 535 nm) were read using the EnVision plate reader. A ratio of dead (cytotoxicity) to live (viability) cells was reported. Data was exported and analyzed in Genedata. Data was normalized using percent of control, no tet (100% viability), and 50 ng/mL tet treated (0% viability), and fit using Smart Fit settings in Genedata.

### Potency Data for Compounds 1 to 1183

The compounds of Formula I are useful as inhibitors of APOL1 activity. Table 5 below illustrates the IC₅₀ of Compounds 1 to 1183 using procedures described above. The procedures above may also be used to determine the potency of any compounds of Formula I. In Table 51 below, the following meanings apply. For IP₅₀ (i.e., IC₅₀ for cell proliferation), “+++” means <0.1 μM; “++” means 0.1-0.5 μM (inclusive); “+” means >0.5-1.0 μM. N.D.=Not determined.

TABLE 51
Potency Data for Compounds 1 to 1042

| Compound | |
|---|---|
| Number | IP₅₀ |
| 1 | +++ |
| 2 | +++ |
| 3 | +++ |
| 4 | +++ |
| 5 | +++ |
| 6 | +++ |
| 7 | +++ |
| 8 | +++ |
| 9 | + |
| 10 | + |
| 11 | +++ |
| 12 | +++ |
| 13 | +++ |
| 14 | ++ |
| 15 | ++ |
| 16 | ++ |
| 17 | +++ |
| 18 | +++ |
| 19 | ++ |
| 20 | ++ |
| 21 | +++ |
| 22 | +++ |
| 23 | +++ |
| 24 | +++ |
| 25 | +++ |
| 26 | +++ |
| 27 | +++ |
| 28 | ++ |
| 29 | +++ |
| 30 | ++ |
| 31 | ++ |
| 32 | +++ |
| 33 | +++ |
| 34 | +++ |
| 35 | ++ |
| 36 | ++ |
| 37 | +++ |
| 38 | ++ |
| 39 | ++ |
| 40 | ++ |
| 41 | +++ |
| 42 | +++ |
| 43 | ++ |
| 44 | +++ |
| 45 | +++ |
| 46 | ++ |
| 47 | ++ |
| 48 | +++ |
| 49 | +++ |
| 50 | +++ |
| 51 | ++ |
| 52 | +++ |
| 53 | + |
| 54 | +++ |
| 55 | ++ |
| 56 | +++ |
| 57 | +++ |
| 58 | ++ |
| 59 | +++ |
| 60 | +++ |
| 61 | ++ |
| 62 | +++ |
| 63 | +++ |
| 64 | +++ |
| 65 | ++ |
| 66 | ++ |
| 67 | +++ |
| 68 | ++ |
| 69 | +++ |
| 70 | +++ |
| 71 | +++ |
| 72 | +++ |
| 73 | +++ |
| 74 | ++ |
| 75 | +++ |
| 76 | ++ |
| 77 | + |
| 78 | ++ |
| 79 | ++ |
| 80 | ++ |
| 81 | ++ |
| 82 | ++ |
| 83 | ++ |
| 84 | +++ |
| 85 | +++ |
| 86 | +++ |
| 87 | ++ |
| 88 | +++ |
| 89 | +++ |
| 90 | ++ |
| 91 | +++ |
| 92 | + |
| 93 | + |
| 94 | ++ |
| 95 | +++ |
| 96 | +++ |
| 97 | +++ |
| 98 | ++ |
| 99 | +++ |
| 100 | ++ |
| 101 | ++ |
| 102 | +++ |
| 103 | ++ |
| 104 | ++ |
| 105 | + |
| 106 | ++ |
| 107 | +++ |
| 108 | ++ |
| 109 | ++ |
| 110 | ++ |
| 111 | ++ |
| 112 | +++ |
| 113 | +++ |
| 114 | ++ |
| 115 | +++ |
| 116 | ++ |
| 117 | +++ |
| 118 | ++ |
| 119 | +++ |
| 120 | ++ |
| 121 | +++ |
| 122 | ++ |
| 123 | +++ |
| 124 | +++ |
| 125 | +++ |
| 126 | +++ |
| 127 | + |
| 128 | ++ |
| 129 | ++ |
| 130 | ++ |
| 131 | ++ |
| 132 | ++ |
| 133 | +++ |
| 134 | +++ |
| 135 | ++ |
| 136 | +++ |
| 137 | +++ |
| 138 | +++ |
| 139 | +++ |
| 140 | +++ |
| 141 | ++ |
| 142 | ++ |
| 143 | +++ |
| 144 | ++ |
| 145 | +++ |
| 146 | +++ |
| 147 | + |
| 148 | ++ |
| 149 | + |
| 150 | ++ |
| 151 | +++ |
| 152 | ++ |
| 153 | +++ |
| 154 | +++ |
| 155 | +++ |
| 156 | +++ |
| 157 | +++ |
| 158 | +++ |
| 159 | ++ |
| 160 | +++ |
| 161 | +++ |
| 162 | +++ |
| 163 | + |
| 164 | + |
| 165 | +++ |
| 166 | + |
| 167 | +++ |
| 168 | + |
| 169 | +++ |
| 170 | + |
| 171 | ++ |
| 172 | ++ |
| 173 | ++ |
| 174 | ++ |
| 175 | + |
| 176 | ++ |
| 177 | + |
| 178 | ++ |
| 179 | +++ |
| 180 | +++ |
| 181 | ++ |
| 182 | ++ |
| 183 | +++ |
| 184 | +++ |
| 185 | +++ |
| 186 | ++ |
| 187 | ++ |
| 188 | ++ |
| 189 | +++ |
| 190 | +++ |
| 191 | +++ |
| 192 | + |
| 193 | + |
| 194 | + |
| 195 | ++ |
| 196 | ++ |
| 197 | ++ |
| 198 | ++ |
| 199 | +++ |
| 200 | +++ |
| 201 | +++ |
| 202 | +++ |
| 203 | +++ |
| 204 | +++ |
| 205 | +++ |
| 206 | +++ |
| 207 | +++ |
| 208 | +++ |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | +++ |
| 219 | +++ |
| 220 | +++ |
| 221 | +++ |
| 222 | +++ |
| 223 | +++ |
| 224 | +++ |
| 225 | +++ |
| 226 | +++ |
| 227 | +++ |
| 228 | +++ |
| 229 | +++ |
| 230 | +++ |
| 231 | ++ |
| 232 | +++ |
| 233 | ++ |
| 234 | +++ |
| 235 | ++ |
| 236 | ++ |
| 237 | ++ |
| 238 | ++ |
| 239 | ++ |
| 240 | ++ |
| 241 | ++ |
| 242 | ++ |
| 243 | ++ |
| 244 | ++ |
| 245 | ++ |
| 246 | +++ |
| 247 | ++ |
| 248 | ++ |
| 249 | +++ |
| 250 | ++ |
| 251 | +++ |
| 252 | +++ |
| 253 | ++ |
| 254 | +++ |
| 255 | + |
| 256 | ++ |
| 257 | ++ |
| 258 | + |
| 259 | +++ |
| 260 | + |
| 261 | ++ |
| 262 | + |
| 263 | + |
| 264 | +++ |
| 265 | +++ |
| 266 | +++ |
| 267 | +++ |
| 268 | +++ |
| 269 | +++ |
| 270 | ++ |
| 271 | +++ |
| 272 | ++ |
| 273 | ++ |
| 274 | +++ |
| 275 | + |
| 276 | + |
| 277 | + |
| 278 | ++ |
| 279 | +++ |
| 280 | ++ |
| 281 | ++ |
| 282 | + |
| 283 | + |
| 284 | + |
| 285 | |
| 286 | + |
| 287 | +++ |
| 288 | +++ |
| 289 | ++ |
| 290 | + |
| 291 | ++ |
| 292 | ++ |
| 293 | + |
| 294 | + |
| 295 | ++ |
| 296 | ++ |
| 297 | + |
| 298 | + |
| 299 | ++ |
| 300 | +++ |
| 301 | ++ |
| 302 | +++ |
| 303 | +++ |
| 304 | +++ |
| 305 | +++ |
| 306 | +++ |
| 307 | +++ |
| 308 | +++ |
| 309 | +++ |
| 310 | ++ |
| 311 | ++ |
| 312 | +++ |
| 313 | +++ |
| 314 | +++ |
| 315 | +++ |
| 316 | +++ |
| 317 | ++ |
| 318 | +++ |
| 319 | +++ |
| 320 | +++ |
| 321 | ++ |
| 322 | ++ |
| 323 | +++ |
| 324 | +++ |
| 325 | +++ |
| 326 | +++ |
| 327 | ++ |
| 328 | ++ |
| 329 | ++ |
| 330 | +++ |
| 331 | ++ |
| 332 | ++ |
| 333 | +++ |
| 334 | ++ |
| 335 | +++ |
| 336 | ++ |
| 337 | +++ |
| 338 | +++ |
| 339 | +++ |
| 340 | +++ |
| 341 | +++ |
| 342 | +++ |
| 343 | +++ |
| 344 | +++ |
| 345 | ++ |
| 346 | +++ |
| 347 | ++ |
| 348 | ++ |
| 349 | +++ |
| 350 | ++ |
| 351 | ++ |
| 352 | +++ |
| 353 | +++ |
| 354 | ++ |
| 355 | +++ |
| 356 | +++ |
| 357 | +++ |
| 358 | +++ |
| 359 | ++ |
| 360 | ++ |
| 361 | ++ |
| 362 | ++ |
| 363 | +++ |
| 364 | ++ |
| 365 | ++ |
| 366 | ++ |
| 367 | ++ |
| 368 | ++ |
| 369 | ++ |
| 370 | ++ |
| 371 | +++ |
| 372 | + |
| 373 | ++ |
| 374 | ++ |
| 375 | +++ |
| 376 | +++ |
| 377 | +++ |
| 378 | ++ |
| 379 | +++ |
| 380 | ++ |
| 381 | ++ |
| 382 | +++ |
| 383 | ++ |
| 384 | ++ |
| 385 | ++ |
| 386 | +++ |
| 387 | ++ |
| 388 | +++ |
| 389 | +++ |
| 390 | +++ |
| 391 | +++ |
| 392 | + |
| 393 | +++ |
| 394 | +++ |
| 395 | +++ |
| 396 | +++ |
| 397 | +++ |
| 398 | +++ |
| 399 | +++ |
| 400 | +++ |
| 401 | +++ |
| 402 | +++ |
| 403 | +++ |
| 404 | +++ |
| 405 | +++ |
| 406 | +++ |
| 407 | +++ |
| 408 | +++ |
| 409 | +++ |
| 410 | +++ |
| 411 | ++ |
| 412 | +++ |
| 413 | +++ |
| 414 | +++ |
| 415 | ++ |
| 416 | ++ |
| 417 | + |
| 418 | ++ |
| 419 | + |
| 420 | +++ |
| 421 | + |
| 422 | +++ |
| 423 | +++ |
| 424 | + |
| 425 | +++ |
| 426 | +++ |
| 427 | +++ |
| 428 | +++ |
| 429 | +++ |
| 430 | +++ |
| 431 | +++ |
| 432 | +++ |
| 433 | +++ |
| 434 | +++ |
| 435 | +++ |
| 436 | +++ |
| 437 | +++ |
| 438 | +++ |
| 439 | +++ |
| 440 | +++ |
| 441 | +++ |
| 442 | +++ |
| 443 | +++ |
| 444 | +++ |
| 445 | +++ |
| 446 | +++ |
| 447 | ++ |
| 448 | ++ |
| 449 | ++ |
| 450 | ++ |
| 451 | ++ |
| 452 | ++ |
| 453 | ++ |
| 454 | ++ |
| 455 | ++ |
| 456 | ++ |
| 457 | + |
| 458 | ++ |
| 459 | +++ |
| 460 | +++ |
| 461 | +++ |
| 462 | +++ |
| 463 | ++ |
| 464 | +++ |
| 465 | +++ |
| 466 | +++ |
| 467 | +++ |
| 468 | +++ |
| 469 | +++ |
| 470 | +++ |
| 471 | +++ |
| 472 | +++ |
| 473 | ++ |
| 474 | +++ |
| 475 | +++ |
| 476 | +++ |
| 477 | +++ |
| 478 | ++ |
| 479 | +++ |
| 480 | ++ |
| 481 | ++ |
| 482 | +++ |
| 483 | ++ |
| 484 | +++ |
| 485 | +++ |
| 486 | ++ |
| 487 | ++ |
| 488 | +++ |
| 489 | |
| 490 | + |
| 491 | ++ |
| 492 | ++ |
| 493 | + |
| 494 | ++ |
| 495 | + |
| 496 | + |
| 497 | ++ |
| 498 | + |
| 499 | +++ |
| 500 | +++ |
| 501 | ++ |
| 502 | ++ |
| 503 | ++ |
| 504 | ++ |
| 505 | +++ |
| 506 | ++ |
| 507 | ++ |
| 508 | ++ |
| 509 | ++ |
| 510 | +++ |
| 511 | +++ |
| 512 | ++ |
| 513 | ++ |
| 514 | ++ |
| 515 | +++ |
| 516 | +++ |
| 517 | ++ |
| 518 | +++ |
| 519 | +++ |
| 520 | +++ |
| 521 | +++ |
| 522 | +++ |
| 523 | +++ |
| 524 | +++ |
| 525 | +++ |
| 526 | ++ |
| 527 | +++ |
| 528 | ++ |
| 529 | +++ |
| 530 | +++ |
| 531 | ++ |
| 532 | ++ |
| 533 | + |
| 534 | +++ |
| 535 | +++ |
| 536 | +++ |
| 537 | +++ |
| 538 | + |
| 539 | |
| 540 | +++ |
| 541 | +++ |
| 542 | +++ |
| 543 | +++ |
| 544 | + |
| 545 | +++ |
| 546 | +++ |
| 547 | +++ |
| 548 | +++ |
| 549 | +++ |
| 550 | +++ |
| 551 | +++ |
| 552 | +++ |
| 553 | +++ |
| 554 | +++ |
| 555 | +++ |
| 556 | +++ |
| 557 | +++ |
| 558 | +++ |
| 559 | +++ |
| 560 | +++ |
| 561 | +++ |
| 562 | +++ |
| 563 | +++ |
| 564 | +++ |
| 565 | +++ |
| 566 | +++ |
| 567 | +++ |
| 568 | +++ |
| 569 | +++ |
| 570 | ++ |
| 571 | ++ |
| 572 | ++ |
| 573 | ++ |
| 574 | + |
| 575 | + |
| 576 | ++ |
| 577 | ++ |
| 578 | +++ |
| 579 | +++ |
| 580 | ++ |
| 581 | ++ |
| 582 | + |
| 583 | ++ |
| 584 | +++ |
| 585 | ++ |
| 586 | + |
| 587 | ++ |
| 588 | + |
| 589 | + |
| 590 | + |
| 591 | + |
| 592 | + |
| 593 | +++ |
| 594 | +++ |
| 595 | +++ |
| 596 | +++ |
| 597 | +++ |
| 598 | ++ |
| 599 | ++ |
| 600 | ++ |
| 601 | +++ |
| 602 | +++ |
| 603 | ++ |
| 604 | ++ |
| 605 | ++ |
| 606 | ++ |
| 607 | ++ |
| 608 | ++ |
| 609 | +++ |
| 610 | +++ |
| 611 | +++ |
| 612 | ++ |
| 613 | ++ |
| 614 | ++ |
| 615 | ++ |
| 616 | + |
| 617 | +++ |
| 618 | + |
| 619 | ++ |
| 620 | ++ |
| 621 | +++ |
| 622 | + |
| 623 | + |
| 624 | + |
| 625 | + |
| 626 | +++ |
| 627 | ++ |
| 628 | ++ |
| 629 | ++ |
| 630 | +++ |
| 631 | +++ |
| 632 | +++ |
| 633 | +++ |
| 634 | +++ |
| 635 | +++ |
| 636 | +++ |
| 637 | +++ |
| 638 | +++ |
| 639 | +++ |
| 640 | ++ |
| 641 | ++ |
| 642 | + |
| 643 | + |
| 644 | +++ |
| 645 | ++ |
| 646 | +++ |
| 647 | +++ |
| 648 | +++ |
| 649 | ++ |
| 650 | ++ |
| 651 | +++ |
| 652 | ++ |
| 653 | ++ |
| 654 | ++ |
| 655 | +++ |
| 656 | ++ |
| 657 | +++ |
| 658 | +++ |
| 659 | ++ |
| 660 | ++ |
| 66 | ++ |
| 662 | +++ |
| 663 | +++ |
| 664 | +++ |
| 665 | ++ |
| 666 | ++ |
| 667 | ++ |
| 668 | + |
| 669 | ++ |
| 670 | ++ |
| 671 | +++ |
| 672 | ++ |
| 673 | + |
| 674 | + |
| 675 | ++ |
| 676 | ++ |
| 677 | + |
| 678 | + |
| 679 | + |
| 680 | + |
| 681 | + |
| 682 | ++ |
| 683 | + |
| 684 | + |
| 685 | + |
| 686 | + |
| 687 | + |
| 688 | + |
| 689 | + |
| 690 | + |
| 691 | |
| 692 | |
| 693 | + |
| 694 | + |
| 695 | + |
| 696 | ++ |
| 697 | +++ |
| 698 | +++ |
| 699 | +++ |
| 700 | +++ |
| 701 | +++ |
| 702 | +++ |
| 703 | +++ |
| 704 | ++ |
| 705 | ++ |
| 706 | +++ |
| 707 | +++ |
| 708 | ++ |
| 709 | ++ |
| 710 | + |
| 711 | ++ |
| 712 | + |
| 713 | ++ |
| 714 | +++ |
| 715 | +++ |
| 716 | ++ |
| 717 | ++ |
| 718 | ++ |
| 719 | ++ |
| 720 | ++ |
| 721 | + |
| 722 | ++ |
| 723 | ++ |
| 724 | +++ |
| 725 | ++ |
| 726 | + |
| 727 | ++ |
| 728 | +++ |
| 729 | +++ |
| 730 | ++ |
| 731 | + |
| 732 | + |
| 733 | ++ |
| 734 | ++ |
| 735 | + |
| 736 | + |
| 737 | + |
| 738 | ++ |
| 739 | + |
| 740 | + |
| 741 | |
| 742 | +++ |
| 743 | ++ |
| 744 | +++ |
| 745 | ++ |
| 746 | + |
| 747 | |
| 748 | ++ |
| 749 | |
| 750 | +++ |
| 751 | |
| 752 | |
| 753 | |
| 754 | +++ |
| 755 | ++ |
| 756 | ++ |
| 757 | +++ |
| 758 | + |
| 759 | ++ |
| 760 | ++ |
| 761 | ++ |
| 762 | +++ |
| 763 | +++ |
| 764 | +++ |
| 765 | +++ |
| 766 | + |
| 767 | +++ |
| 768 | +++ |
| 769 | +++ |
| 770 | ++ |
| 771 | ++ |
| 772 | +++ |
| 773 | +++ |
| 774 | + |
| 775 | +++ |
| 776 | +++ |
| 777 | +++ |
| 778 | + |
| 779 | + |
| 780 | +++ |
| 781 | + |
| 782 | + |
| 783 | ++ |
| 784 | ++ |
| 785 | + |
| 786 | + |
| 787 | + |
| 788 | ++ |
| 789 | + |
| 790 | ++ |
| 791 | +++ |
| 792 | ++ |
| 793 | ++ |
| 794 | ++ |
| 795 | |
| 796 | ++ |
| 797 | ++ |
| 798 | +++ |
| 799 | +++ |
| 800 | +++ |
| 801 | + |
| 802 | ++ |
| 803 | ++ |
| 804 | +++ |
| 805 | + |
| 806 | ++ |
| 807 | ++ |
| 808 | + |
| 809 | ++ |
| 810 | ++ |
| 811 | + |
| 812 | ++ |
| 813 | ++ |
| 814 | |
| 815 | + |
| 816 | + |
| 817 | +++ |
| 818 | +++ |
| 819 | +++ |
| 820 | +++ |
| 821 | ++ |
| 822 | ++ |
| 823 | ++ |
| 824 | +++ |
| 825 | +++ |
| 826 | +++ |
| 827 | +++ |
| 828 | +++ |
| 829 | +++ |
| 830 | +++ |
| 831 | +++ |
| 832 | +++ |
| 833 | +++ |
| 834 | +++ |
| 835 | +++ |
| 836 | +++ |
| 837 | +++ |
| 838 | + |
| 839 | +++ |
| 840 | +++ |
| 841 | +++ |
| 842 | +++ |
| 843 | +++ |
| 844 | ++ |
| 845 | ++ |
| 846 | +++ |
| 847 | +++ |
| 848 | +++ |
| 849 | ++ |
| 850 | ++ |
| 851 | +++ |
| 852 | +++ |
| 853 | ++ |
| 854 | +++ |
| 855 | +++ |
| 856 | +++ |
| 857 | +++ |
| 858 | +++ |
| 859 | +++ |
| 860 | +++ |
| 861 | ++ |
| 862 | ++ |
| 863 | ++ |
| 864 | ++ |
| 865 | ++ |
| 866 | ++ |
| 867 | ++ |
| 868 | |
| 869 | + |
| 870 | +++ |
| 871 | +++ |
| 872 | +++ |
| 873 | +++ |
| 874 | +++ |
| 875 | +++ |
| 876 | +++ |
| 877 | +++ |
| 878 | +++ |
| 879 | +++ |
| 880 | +++ |
| 881 | +++ |
| 882 | +++ |
| 883 | +++ |
| 884 | +++ |
| 885 | +++ |
| 886 | +++ |
| 887 | +++ |
| 888 | +++ |
| 889 | +++ |
| 890 | +++ |
| 891 | +++ |
| 892 | +++ |
| 893 | +++ |
| 894 | +++ |
| 895 | +++ |
| 896 | +++ |
| 897 | +++ |
| 898 | +++ |
| 899 | ++ |
| 900 | +++ |
| 901 | +++ |
| 902 | +++ |
| 903 | +++ |
| 904 | +++ |
| 905 | +++ |
| 906 | +++ |
| 907 | +++ |
| 908 | +++ |
| 909 | +++ |
| 910 | ++ |
| 911 | +++ |
| 912 | +++ |
| 913 | +++ |
| 914 | +++ |
| 915 | +++ |
| 916 | +++ |
| 917 | +++ |
| 918 | +++ |
| 919 | +++ |
| 920 | ++ |
| 921 | +++ |
| 922 | +++ |
| 923 | +++ |
| 924 | +++ |
| 925 | ++ |
| 926 | +++ |
| 927 | +++ |
| 928 | +++ |
| 929 | +++ |
| 930 | +++ |
| 931 | +++ |
| 932 | +++ |
| 933 | +++ |
| 934 | +++ |
| 935 | +++ |
| 936 | +++ |
| 937 | +++ |
| 938 | +++ |
| 939 | +++ |
| 940 | +++ |
| 941 | +++ |
| 942 | +++ |
| 943 | +++ |
| 944 | +++ |
| 945 | +++ |
| 946 | +++ |
| 947 | +++ |
| 948 | +++ |
| 949 | +++ |
| 950 | +++ |
| 951 | + |
| 952 | +++ |
| 953 | +++ |
| 954 | +++ |
| 955 | +++ |
| 956 | +++ |
| 957 | +++ |
| 958 | +++ |
| 959 | +++ |
| 960 | +++ |
| 961 | +++ |
| 962 | +++ |
| 963 | +++ |
| 964 | +++ |
| 965 | +++ |
| 966 | +++ |
| 967 | +++ |
| 968 | +++ |
| 969 | +++ |
| 970 | +++ |
| 971 | +++ |
| 972 | +++ |
| 973 | +++ |
| 974 | +++ |
| 975 | +++ |
| 976 | +++ |
| 977 | +++ |
| 978 | +++ |
| 979 | +++ |
| 980 | ++ |
| 981 | +++ |
| 982 | +++ |
| 983 | +++ |
| 984 | +++ |
| 985 | +++ |
| 986 | +++ |
| 987 | +++ |
| 988 | +++ |
| 989 | +++ |
| 990 | +++ |
| 991 | +++ |
| 992 | +++ |
| 993 | ++ |
| 994 | +++ |
| 995 | +++ |
| 996 | +++ |
| 997 | +++ |
| 998 | +++ |
| 999 | +++ |
| 1000 | + |
| 1001 | ++ |
| 1002 | +++ |
| 1003 | +++ |
| 1004 | ++ |
| 1005 | +++ |
| 1006 | ++ |
| 1007 | + |
| 1008 | + |
| 1009 | + |
| 1010 | +++ |
| 1011 | +++ |
| 1012 | +++ |
| 1013 | +++ |
| 1014 | +++ |
| 1015 | +++ |
| 1016 | ++ |
| 1017 | ++ |
| 1018 | ++ |
| 1019 | + |
| 1020 | + |
| 1021 | ++ |
| 1022 | +++ |
| 1023 | + |
| 1024 | +++ |
| 1025 | +++ |
| 1026 | +++ |
| 1027 | +++ |
| 1028 | +++ |
| 1029 | ++ |
| 1030 | ++ |
| 1031 | +++ |
| 1032 | +++ |
| 1033 | +++ |
| 1034 | +++ |
| 1035 | +++ |
| 1036 | +++ |
| 1037 | +++ |
| 1038 | +++ |
| 1039 | +++ |
| 1040 | +++ |
| 1041 | +++ |
| 1042 | ++ |
| 1043 | +++ |
| 1044 | +++ |
| 1045 | +++ |
| 1046 | +++ |
| 1047 | +++ |
| 1048 | +++ |
| 1049 | +++ |
| 1050 | +++ |
| 1051 | +++ |
| 1052 | +++ |
| 1053 | +++ |
| 1054 | +++ |
| 1055 | +++ |
| 1056 | ++ |
| 1057 | ++ |
| 1058 | ++ |
| 1059 | ++ |
| 1060 | ++ |
| 1061 | ++ |
| 1062 | ++ |
| 1063 | ++ |
| 1064 | ++ |
| 1065 | + |
| 1066 | +++ |
| 1067 | +++ |
| 1068 | +++ |
| 1069 | ++ |
| 1070 | ++ |
| 1071 | ++ |
| 1072 | ++ |
| 1073 | +++ |
| 1074 | +++ |
| 1075 | ++ |
| 1076 | ++ |
| 1077 | ++ |
| 1078 | ++ |
| 1079 | ++ |
| 1080 | ++ |
| 1081 | ++ |
| 1082 | ++ |
| 1083 | + |
| 1084 | + |
| 1085 | + |
| 1086 | + |
| 1087 | + |
| 1088 | + |
| 1089 | + |
| 1090 | + |
| 1091 | + |
| 1092 | + |
| 1093 | + |
| 1094 | + |
| 1095 | + |
| 1096 | + |
| 1097 | + |
| 1098 | + |
| 1099 | + |
| 1100 | + |
| 1101 | + |
| 1102 | + |
| 1103 | + |
| 1104 | + |
| 1105 | + |
| 1106 | + |
| 1107 | + |
| 1108 | + |
| 1109 | + |
| 1110 | + |
| 1111 | + |
| 1112 | + |
| 1113 | + |
| 1114 | + |
| 1115 | + |
| 1116 | + |
| 1117 | + |
| 1118 | + |
| 1119 | + |
| 1120 | + |
| 1121 | + |
| 1122 | + |
| 1123 | + |
| 1124 | + |
| 1125 | + |
| 1126 | +++ |
| 1127 | +++ |
| 1128 | +++ |
| 1129 | ++ |
| 1130 | ++ |
| 1131 | ++ |
| 1132 | ++ |
| 1133 | ++ |
| 1134 | ++ |
| 1135 | ++ |
| 1136 | ++ |
| 1137 | ++ |
| 1138 | ++ |
| 1139 | + |
| 1140 | + |
| 1141 | + |
| 1142 | + |
| 1143 | + |
| 1144 | + |
| 1145 | + |
| 1146 | + |
| 1147 | + |
| 1148 | + |
| 1149 | + |
| 1150 | + |
| 1151 | + |
| 1152 | + |
| 1153 | + |
| 1154 | + |
| 1155 | + |
| 1156 | + |
| 1157 | ++ |
| 1158 | + |
| 1159 | +++ |
| 1160 | ++ |
| 1161 | ++ |
| 1162 | ++ |
| 1163 | ++ |
| 1164 | ++ |
| 1165 | ++ |
| 1166 | ++ |
| 1167 | ++ |
| 1168 | + |
| 1169 | ++ |
| 1170 | ++ |
| 1171 | + |
| 1172 | + |
| 1173 | ++ |
| 1174 | ++ |
| 1175 | ++ |
| 1176 | + |
| 1177 | +++ |
| 1178 | +++ |
| 1179 | +++ |
| 1180 | + |
| 1181 | + |
| 1182 | + |
| 1183 | + |

### OTHER EMBODIMENTS

This disclosure provides merely non-limiting example embodiments of the disclosed subject matter. One skilled in the art will readily recognize from the disclosure and claims, that various changes, modifications and variations can be made therein without departing from the spirit and scope of the disclosure as defined in the following claims.

1. A compound represented by the following structural formula: 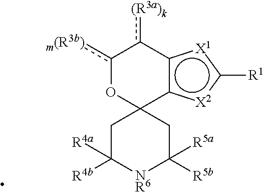
a tautomer thereof, a deuterated derivative of that compound or tautomer, or a pharmaceutically acceptable salt of any of the foregoing, wherein:
X¹ and X² are chosen from —S—, —S(═O)₂—, —S(═O)—, and —CR², wherein:
one of X¹ and X² is chosen from —S—, —S(═O)₂—, and —S(═O)—;
when X¹ is —S—, —S(═O)₂—, or —S(═O)—, then X² is —CR²; and
when X² is —S—, —S(═O)₂—, or —S(═O)—, then X¹ is —CR²;
R¹ is chosen from cyano, halogen, C₁-C₄ alkyl, C₁-C₄ haloalkyl, and C₃-C₆ cycloalkyl groups, wherein:
the C₁-C₄ alkyl of R¹ is optionally substituted with 1 to 3 groups independently chosen from —OH and C₁-C₄ alkoxy groups;
R² is chosen from hydrogen, C₁-C₆ alkyl, —C(═O)O(C₁-C₄ alkyl), —C(═O)NRⁿR^{o}, and halogen groups, wherein:
the C₁-C₆ alkyl of R² is optionally substituted with 1 to 3 groups independently chosen from —OH, halogen, and C₁-C₄ alkoxy groups; and
Rⁿ and R^{o} are independently chosen from hydrogen, C₁-C₄ alkyl, C₁-C₄ haloalkyl, C₃-C₆ cycloalkyl, and —(C₁-C₄ alkylene)R^{p} groups, wherein R^{p} is chosen from C₃-C₆ cycloalkyl groups; or
R¹ and R², together with the carbon atoms to which they are attached, form a C₆ aryl group;
k is chosen from 0, 1, and 2;
m is chosen from 0, 1, and 2;
each R^{3a} is independently chosen from —OH, —CN, —NR^{a1}R^{a2}, C₁-C₆ alkyl, C₁-C₆ haloalkyl, C₁-C₆ alkoxy, —OC(═O)(C₁-C₄ alkyl), 6- to 8-membered aryl, 6- to 8-membered heteroaryl, and halogen groups, wherein:
each R^{a1} and R^{a2} is independently chosen from hydrogen, C₁-C₄ alkyl, and —C(═O)(C₁-C₄ alkyl) groups; or
two R^{3a} taken together form an oxo group; or
two R^{3a}, together with the carbon atom to which they are attached, form a C₃-C₆ cycloalkyl group;
each R^{3b} is independently chosen from C₁-C₄ alkyl groups, wherein:
the C₁-C₄ alkyl of R^{3b} is optionally substituted with 1 to 3 groups independently chosen from —OH, halogen, and C₁-C₄ alkoxy groups; or
one R^{3a} and one R^{3b}, together with the carbon atoms to which they are attached, form a C₃-C₆ cycloalkyl group;
R^{4a}, R^{4b}, R^{5a}, and R^{5b} are each independently chosen from hydrogen and C₁-C₄ alkyl groups;
R⁶ is chosen from C₁-C₆ alkyl, —C(═O)O(C₁-C₄ alkyl), and 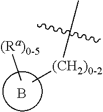
groups, wherein:
the C₁-C₆ alkyl of R⁶ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
the C₆ aryl and —O—(C₆ aryl) groups are each optionally substituted with 1 to 3 groups independently chosen from halogen and C₁-C₄ haloalkyl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
R^{a}, for each occurrence, is independently chosen from
halogen,
cyano,
C₁-C₈ alkyl,
C₁-C₆ haloalkyl,
C₂-C₈ alkenyl,
C₁-C₆ haloalkenyl,
C₁-C₆ alkoxy,
C₁-C₆ haloalkoxy,
C₃-C₁₂ carbocyclyl,
C₆ and C₁₀ aryl,
3- to 12-membered heterocyclyl,
5- to 10-membered heteroaryl,
—C(═O)NR^{h}Rⁱ,
—C(═O)OR^{k},
—C(═O)(C₁-C₄ alkylene)OR^{k},
—C(═O)R^{k},
—C(═O)(C₁-C₄ alkylene)S(═O)ₚR^{k},
—C(═O)(C₁-C₄ alkylene)S(═O)ₚNR^{h}Rⁱ,
—C(═O)(C₁-C₄ alkylene)NRⁱS(═O)ₚR^{k},
—C(═O)(C₁-C₄ alkylene)NR^{h}C(═O)R^{k},
—C(═O)C(═O)R^{k},
—NR^{h}Rⁱ,
—NH(CH₂)_{q}CHR^{h}Rⁱ,
—NH(CH₂)_{q}NR^{h}Rⁱ,
—NR^{h}C(═O)R^{k},
—NR^{h}C(═O)OR^{k},
—NR^{h}C(═O)(C₁-C₄ alkylene)OR^{k},
—NR^{h}C(═O)O(C₁-C₄ alkylene)R^{k},
—NR^{h}C(═O)NRⁱR^{j},
—NR^{h}C(═O)(C₁-C₄ alkylene)NRⁱS(═O)ₚR^{k},
—NR^{h}S(═O)ₚR^{k},
—NR^{h}C(═O)(C₁-C₄ alkylene)S(═O)ₚR^{k},
—NR^{h}S(═O)ₚ(C₁-C₄ alkylene)C(═O)OR^{k},
—NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups),
—NR^{h}C(═O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups),
—OR^{k},
—OC(═O)R^{k},
—OC(═O)OR^{k},
—OC(═O)NR^{h}Rⁱ, —[O(CH₂)_{q}]ᵣO(C₁-C₆ alkyl),
—S(═O)ₚR^{k}, and
—S(═O)ₚNR^{h}Rⁱ groups,
wherein:
the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k}, —NR^{h}C(═O)O(C₁-C₄ alkylene)R^{k}, —NR^{h}C(═O)(C₁-C₄ alkylene)OR^{k}, _NR^{h}S(═O)ₚ(C₁-C₄ alkylene)C(═O)OR^{k}, and —NR^{h}C(═O)(C₁-C₄ alkylene)NRⁱS(═O)ₚR^{k} is optionally substituted with 1 to 3 groups independently chosen from —OH,
the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₆ alkyl (optionally substituted with 1 to 3 R^{m} groups), —C(═O)R^{k}, —C(═O)OR^{k}, —NR^{h}Rⁱ, —OR^{k}, —S(═O)ₚR^{k}, —S(═O)ₚNR^{h}Rⁱ, and 5- to 10-membered heterocyclyl groups, wherein:
R^{h}, Rⁱ, and R^{j}, for each occurrence, are each independently chosen from hydrogen, C₁-C₆ alkyl (optionally substituted with 1 to 4 R^{m} groups), C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 3 groups independently chosen from halogen, cyano, —OH, C₁-C₄ alkoxy,
—C(═O)NH(C₁-C₄ alkyl), 5- to 10-membered heteroaryl (optionally substituted with 1 to 3 R^{m} groups), and 5- to 10-membered heterocyclyl (optionally substituted with 1 to 3 R^{m} groups) groups;
R^{k}, for each occurrence, is independently chosen from hydrogen, NH₂, (optionally substituted with 1 or 2 groups chosen from C₁-C₃ alkyl), C₁-C₆ alkyl, benzyl, C₆ aryl, C₃-C₆ carbocyclyl, 5- to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 4- to 10-membered heterocyclyl (optionally substituted with 1 to 3 —OH groups), 5- to 10-membered aryl (optionally substituted with 1 to 3 groups selected from C₁-C₄ alkyl and halogen), and 5- to-10-membered heteroaryl (optionally substituted with 1 to 3 —OH groups) groups; and
the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₆ alkyl (optionally substituted by 1 to 3 —OH groups), C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl groups (optionally substituted with 1 to 3 halogen groups); and
the 5- to 10-membered heteroaryl and 5- to 10-membered heterocyclyl of any one of R^{k} are each optionally substituted with 1 to 3 groups independently chosen from halogen, oxo, cyano, —C(═O)CH₃, —NH₂, —OH, C₁-C₄ alkyl (optionally substituted by 1 to 3 —OH groups), C₁-C₄ haloalkyl, 5- to 10-membered heterocyclyl, and C₁-C₄ alkoxy groups;
R^{m}, for each occurrence, is independently chosen from halogen, cyano, oxo, —NH₂, C₁-C₆ alkyl, C₁-C₆ alkoxy, —C(═O)R^{k},
—S(═O)ₚR^{k}, —OR^{k}, and 5- to 10-membered heterocyclyl groups, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
p, for each occurrence, is an integer independently chosen from 1 and 2; and
q and r, for each occurrence, is an integer independently chosen from 0, 1, 2, and 3.

2. The compound, tautomer, deuterated derivative, or pharmaceutically acceptable salt according to claim 1, wherein the compound is represented by one of the following structural formulae: 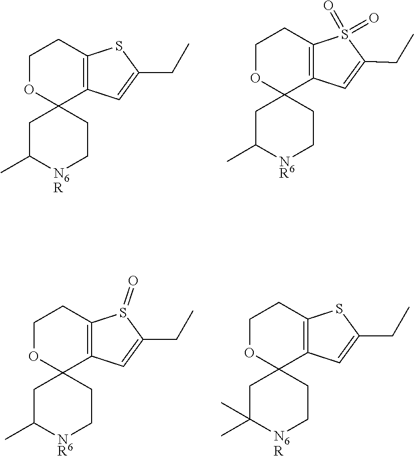
a tautomer thereof, a deuterated derivative of that compound or tautomer, or a pharmaceutically acceptable salt of any of the foregoing, wherein R⁶ is as defined in claim 1.

3. The compound, tautomer, deuterated derivative, or pharmaceutically acceptable salt according to claim 1, wherein the compound is represented by one of the following structural formulae: 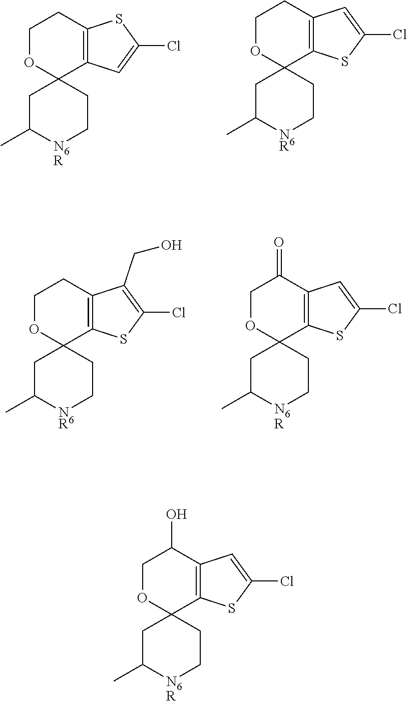
a tautomer thereof, a deuterated derivative of that compound or tautomer, or a pharmaceutically acceptable salt of any of the foregoing, wherein R⁶ is as defined in claim 1.

4. The compound, tautomer, deuterated derivative, or pharmaceutically acceptable salt according to claim 1, wherein the compound is represented by one of the following structural formulae: 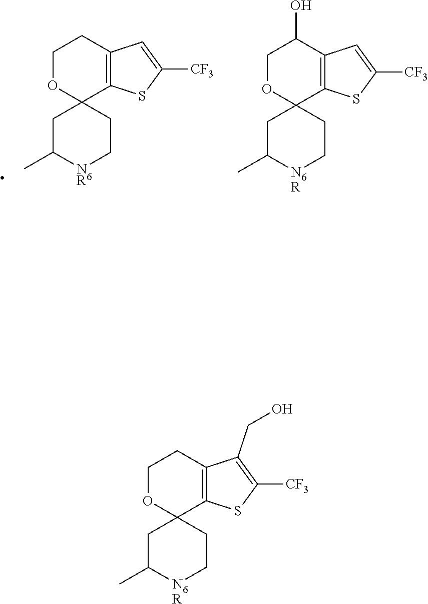
a tautomer thereof, a deuterated derivative of that compound or tautomer, or a pharmaceutically acceptable salt of any of the foregoing, wherein R⁶ is as defined in claim 1.

5. The compound, tautomer, deuterated derivative, or pharmaceutically acceptable salt according to claim 1, wherein the compound is represented by one of the following structural formulae: 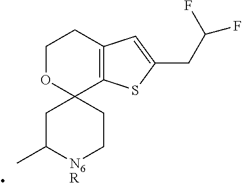
a tautomer thereof, a deuterated derivative of that compound or tautomer, or a pharmaceutically acceptable salt of any of the foregoing, wherein R⁶ is as defined in claim 1.

6. The compound, tautomer, deuterated derivative, or pharmaceutically acceptable salt according to claim 1, wherein the variable X¹ is chosen from S, S(═O), and S(═O)₂ and the variable X² is —CR².

7. The compound, tautomer, deuterated derivative, or pharmaceutically acceptable salt according to claim 1, wherein the variable X² is chosen from S, S(═O), and S(═O)₂ and the variable X¹ is —CR².

8. The compound, tautomer, deuterated derivative, or pharmaceutically acceptable salt according to any one of claims 1, 6 and 7, wherein the variable R² is hydrogen.

9. The compound, tautomer, deuterated derivative, or pharmaceutically acceptable salt according to claim 1, wherein the variable X¹ is S, the variable X² is —CR², and the variable R² is hydrogen.

9. The compound, tautomer, deuterated derivative, or pharmaceutically acceptable salt according to claim 1, wherein the variable X¹ is —CR², the variable X² is S, and the variable R² is hydrogen.

10. The compound, tautomer, deuterated derivative, or pharmaceutically acceptable salt according to any one of claims 1, 6, and 7, wherein R² is chosen from hydrogen, halogen, —CH₃, —CH₂OH,
—CH₂(OH)CH₃, and —C(═O)NRⁿR^{o}, wherein
Rⁿ and R^{o} are independently chosen from hydrogen, —C₁-C₄ alkyl, —C₁-C₄ haloalkyl, —C₃-C₆ cycloalkyl, and —(C₁-C₄ alkylene)R^{p} groups, and wherein
R^{p} is chosen from —C₃-C₆ cycloalkyl groups.

11. The compound, tautomer, deuterated derivative, or pharmaceutically acceptable salt according to claim 10, wherein R² is chosen from halogen.

12. The compound, tautomer, deuterated derivative, or pharmaceutically acceptable salt according to claim 11, wherein R² is chosen from Br and Cl.

13. The compound, tautomer, deuterated derivative, or pharmaceutically acceptable salt according to claim 10, wherein the variable R² is chosen from —CH₃, —CH₂OH, and CH(OH)CH₃.

14. The compound, tautomer, deuterated derivative, or pharmaceutically acceptable salt according to claim 10, wherein R² is —C(═O)NRⁿR^{o}, and wherein Rⁿ is hydrogen and R^{o} is CH₃.

15. The compound, tautomer, deuterated derivative, or pharmaceutically acceptable salt according to claim 10, wherein R² is —C(═O)NRⁿR^{o}, and wherein Rⁿ is hydrogen and R^{o} is CH₃ substituted with a cyclopropyl group.

16. The compound, tautomer, deuterated derivative, or pharmaceutically acceptable salt according to claim 10, wherein R² is —C(═O)NRⁿR^{o}, and wherein Rⁿ is hydrogen and R^{o} is —CH₂CH₂CH₃.

17. The compound, tautomer, deuterated derivative, or pharmaceutically acceptable salt according to claim 10, wherein R² is —C(═O)NRⁿR^{o}, and wherein Rⁿ is hydrogen and R^{o} is —CH₂CF₂.

18. The compound, tautomer, deuterated derivative, or pharmaceutically acceptable salt according to any one of claims 1 and 6-17, wherein R¹ is chosen from cyano, halogen, C₁-C₄ alkyl (optionally substituted with 1 to 3 groups independently chosen from —OH and C₁-C₄ alkoxy groups), C₁-C₄ haloalkyl, and C₃-C₆ cycloalkyl groups.

19. The compound, tautomer, deuterated derivative, or pharmaceutically acceptable salt according to claim 18, wherein the variable R¹ is chosen from —CN, —Br, —Cl, CH₃, CH₂OH, —CH₂CH₃, —CH₂CH₂CH3, tert-butyl, —CH₂CF₂, —CF₂, —CF₃, —CF₂CF₂, —CH₂OCH₃, —CH₂OCH₂CH₃, cyclopropyl, and cyclobutyl.

20. The compound, tautomer, deuterated derivative, or pharmaceutically acceptable salt according to claim 18, wherein the variable R¹ is CF₃.

21. The compound, tautomer, deuterated derivative, or pharmaceutically acceptable salt according to claim 18, wherein the variable R¹ is Cl.

22. The compound, tautomer, deuterated derivative, or pharmaceutically acceptable salt according to claim 18, wherein the variable R¹ is —CH₂OH.

23. The compound, tautomer, deuterated derivative, or pharmaceutically acceptable salt according to claim 18, wherein the variable R¹ is —CH₂CF₃.

24. The compound, tautomer, deuterated derivative, or pharmaceutically acceptable salt according to claim 18, wherein the variable R¹ is —CF₂CF₃.

25. The compound, tautomer, deuterated derivative, or pharmaceutically acceptable salt according to claim 18, wherein the variable R¹ is —CH₂(OH)CH₃.

26. The compound, tautomer, deuterated derivative, or pharmaceutically acceptable salt according to claim 18, wherein the variable R¹ comprises deuterium.

27. The compound, tautomer, deuterated derivative, or pharmaceutically acceptable salt according to claim 26, wherein the variable R¹ is CD-OCH₂CH₃.

28. The compound, tautomer, deuterated derivative, or pharmaceutically acceptable salt according to any one of claims 1, 6, and 7 wherein the variables R¹ and R², together with the carbon atoms to which they are attached, form a C₆ aryl group.

29. The compound, tautomer, deuterated derivative, or pharmaceutically acceptable salt according to any one of claims 1, 6, and 7 wherein the variable R¹ is —Cl and the variable R² is —CH₂OH.

30. The compound, tautomer, deuterated derivative, or pharmaceutically acceptable salt according to any one of claims 1, 6, and 7 wherein the variable R¹ is Cl and the variable R² is —CH₃.

31. The compound, tautomer, deuterated derivative, or pharmaceutically acceptable salt according to any one of claims 1, 6, and 7 wherein the variable R¹ is Cl and the variable R² is hydrogen.

32. The compound, tautomer, deuterated derivative, or pharmaceutically acceptable salt according to any one of claims 1, 6, and 7 wherein the variable R¹ is —Cl and the variable R² is —Cl.

33. The compound, tautomer, deuterated derivative, or pharmaceutically acceptable salt according to any one of claims 1, 6, and 7 wherein the variable R¹ is —CF₃ and the variable R² is hydrogen.

34. The compound, tautomer, deuterated derivative, or pharmaceutically acceptable salt according to any one of claims 1, 6, and 7 wherein

35. The compound, tautomer, deuterated derivative, or pharmaceutically acceptable salt according to any one of claims 1, 6, and 7 wherein —CF₂CF₃ and the variable R² is hydrogen.

36. The compound, tautomer, deuterated derivative, or pharmaceutically acceptable salt according to any one of claims 1, 6, and 7 wherein —CH₂CF₃ and the variable R² is hydrogen.

37. The compound, tautomer, deuterated derivative, or pharmaceutically acceptable salt according to any one of claims 1, 6, and 7 wherein the variable R¹ is —CF₂ and the variable R² is —CH₂OH.

38. The compound, tautomer, deuterated derivative, or pharmaceutically acceptable salt according to any one of claims 1, 6, and 7 wherein the variable R¹ is —CF₃ and the variable R² is —CH₂OH.

39. The compound, tautomer, deuterated derivative, or pharmaceutically acceptable salt according to any one of claims 1, 6, and 7 wherein the variable R¹ is —CF₃ and the variable R² is —CH₂(OH)CH₃.

40. The compound, tautomer, deuterated derivative, or pharmaceutically acceptable salt according to any one of claims 1, 6, and 7 wherein the variable R¹ is —CF₃ and the variable R² is —Cl.

41. The compound, tautomer, deuterated derivative, or pharmaceutically acceptable salt according to any one of claims 1, 6, and 7 wherein the variable R¹ is CH₃ and the variable R² is hydrogen.

42. The compound, tautomer, deuterated derivative, or pharmaceutically acceptable salt according to any one of claims 1, 6, and 7 wherein the variable R¹ is CH₂CH₃ and the variable R² is hydrogen.

43. The compound, tautomer, deuterated derivative, or pharmaceutically acceptable salt according to any one of claims 1, 6, and 7 wherein the variable R¹ is CH₂OH and the variable R² is hydrogen.

44. The compound, tautomer, deuterated derivative, or pharmaceutically acceptable salt according to any one of claims 1, and 6-43, wherein the variables m and k are zero.

45. The compound, tautomer, deuterated derivative, or pharmaceutically acceptable salt according to any one of claims 1, and 6-43, wherein the variable variable m is zero, and the variable k is one.

46. The compound, tautomer, deuterated derivative, or pharmaceutically acceptable salt according to any one of claims 1, and 6-43, wherein the variable m is zero, and the variable k is two.

47. The compound, tautomer, deuterated derivative, or pharmaceutically acceptable salt according to any one of claims 1, and 6-43, wherein the variable variable m is one, and the variable k is zero.

48. The compound, tautomer, deuterated derivative, or pharmaceutically acceptable salt according to any one of claims 1, and 6-43, wherein the variable m is two, and the variable k is zero.

49. The compound, tautomer, deuterated derivative, or pharmaceutically acceptable salt according to any one of claims 1, and 6-43, wherein the variable m is zero, the variable k is one or two, and each R^{3a} variable is independently chosen from —OH, F, F₂, CF₂, —OCH₃, —OCH₂CH₃, —OCH₂(CH₃)₂, —OC(═O)CH₃, NH₂, NHC(═O)CH₃, CN,

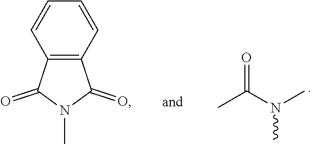

50. The compound, tautomer, deuterated derivative, or pharmaceutically acceptable salt according to any one of claims 1, and 6-43, wherein the variable m is zero, the variable k is one, and the variable R^{3a} is independently chosen from —OH, F, F₂, CF₂, —OCH₃, —OCH₂CH₃, —OCH₂(CH₃)₂, —OC(═O)CH₃, NH₂, NHC(═O)CH₃, CN,

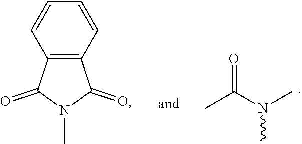

50. The compound, tautomer, deuterated derivative, or pharmaceutically acceptable salt according to any one of claims 1, and 6-43, wherein the variable m is zero, the variable k is one, and the variable R^{3a} is —OH.

51. The compound, tautomer, deuterated derivative, or pharmaceutically acceptable salt according to any one of claims 1, and 6-43, wherein the variable m is zero, the variable k is one, and the variable R^{3a} is chosen from —OCH₃ and —OCH₂CH₃.

52. The compound, tautomer, deuterated derivative, or pharmaceutically acceptable salt according to any one of claims 1, and 6-43, wherein the variable m is zero, the variable k is two, and two R^{3a} variables taken together to form ═O.

53. The compound, tautomer, deuterated derivative, or pharmaceutically acceptable salt according to claim 1, wherein two R^{3a} variables are present and are chosen from:
—CF₂ and —OH;
—CF₂ and —CH₃;
—OH and —CH₃; and
—OH and phenyl.

54. The compound, tautomer, deuterated derivative, or pharmaceutically acceptable salt according to any one of claims 1 and 6 to 53, wherein three of variables R^{4a}, R^{4b}, R^{5a}, and R^{5b} are hydrogen and the remaining variable is chosen from C₁-C₄ alkyl groups.

55. The compound, tautomer, deuterated derivative, or pharmaceutically acceptable salt according to any one of claims 1 and 6 to 53, wherein three of variables R^{4a}, R^{4b}, R^{5a}, and R^{5b} are hydrogen and the remaining variable is CH₃.

56. The compound, tautomer, deuterated derivative, or pharmaceutically acceptable salt according to any one of claims 6 to 55 wherein R⁶ is chosen from C₁-C₆ alkyl, —C(═O)O(C₁-C₄alkyl), and 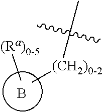
groups, wherein:
the C₁-C₆ alkyl of R⁶ is optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl, —O—(C₆ aryl), 5- to 10-membered heterocyclyl, and 5- to 10-membered heteroaryl groups, wherein:
the C₆ aryl and —O—(C₆ aryl) groups are each optionally substituted with 1 to 3 groups independently chosen from halogen and C₁-C₄ haloalkyl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups; wherein:
R^{a}, for each occurrence, is independently chosen from
halogen,
cyano,
C₁-C₈ alkyl,
C₁-C₆ haloalkyl,
C₂-C₈ alkenyl,
C₁-C₆ haloalkenyl,
C₁-C₆ alkoxy,
C₁-C₆ haloalkoxy,
C₃-C₁₂ carbocyclyl,
C₆ and C₁₀ aryl,
3- to 12-membered heterocyclyl,
5- to 10-membered heteroaryl,
—C(═O)NR^{h}Rⁱ,
—C(═O)OR^{k},
—C(═O)(C₁-C₄ alkylene)OR^{k},
—C(═O)R^{k},
—C(═O)(C₁-C₄ alkylene)S(═O)ₚR^{k},
—C(═O)(C₁-C₄ alkylene)S(═O)ₚNR^{h}Rⁱ,
—C(═O)(C₁-C₄ alkylene)NRⁱS(═O)ₚR^{k},
—C(═O)(C₁-C₄ alkylene)NR^{h}C(═O)R^{k},
—C(═O)C(═O)R^{k},
—NR^{h}Rⁱ,
—NH(CH₂)_{q}CHR^{h}Rⁱ,
—NH(CH₂)_{q}NR^{h}Rⁱ,
—NR^{h}C(═O)R^{k},
—NR^{h}C(═O)OR^{k},
—NR^{h}C(═O)(C₁-C₄ alkylene)OR^{k},
—NR^{h}C(═O)O(C₁-C₄ alkylene)R^{k},
—NR^{h}C(═O)NRⁱR^{j},
—NR^{h}C(═O)(C₁-C₄ alkylene)NRⁱS(═O)ₚR^{k},
—NR^{h}S(═O)ₚR^{k},
—NR^{h}C(═O)(C₁-C₄ alkylene)S(═O)ₚR^{k},
—NR^{h}S(═O)ₚ(C₁-C₄ alkylene)C(═O)OR^{k},
—NR^{h}C(═O)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups),
—NR^{h}C(═O)(C₁-C₆ alkylene)[O(CH₂)_{q}]ᵣOC(═O)NR^{h}Rⁱ(CH₂)_{q}[O(CH₂)_{q}]ᵣ(C₁-C₆ alkyl) (optionally substituted by 1 to 3 R^{m} groups),
—OR^{k},
—OC(═O)R^{k},
—OC(═O)OR^{k},
—OC(═O)NR^{h}Rⁱ, —[O(CH₂)_{q}]ᵣO(C₁-C₆ alkyl),
—S(═O)ₚR^{k}, and
—S(═O)ₚNR^{h}Rⁱ groups,
wherein:
the C₁-C₄ alkylene in each of —C(═O)(C₁-C₄ alkylene)S(═O)ₚR^{k}, —C(═O)(C₁-C₄ alkylene)OR^{k}, —C(═O)(C₁-C₄ alkylene)S(═O)ₚNR^{h}Rⁱ, —C(═O)(C₁-C₄ alkylene)NRⁱS(═O)ₚR^{k}, —C(═O)(C₁-C₄ alkylene)NR^{h}C(═O)R^{k},
—NR^{h}C(═O)O(C₁-C₄ alkylene)R^{k}, —NR^{h}C(═O)(C₁-C₄ alkylene)OR^{k}, —NR^{h}S(═O)ₚ(C₁-C₄ alkylene)C(═O)OR^{k}, and —NR^{h}C(═O)(C₁-C₄ alkylene)NRⁱS(═O)ₚR^{k} is optionally substituted with 1 to 3 groups independently chosen from —OH,
the C₁-C₈ alkyl, the C₁-C₆ haloalkyl, the C₁-C₆ alkoxy, and the C₂-C₈ alkenyl of R^{a} are each optionally substituted with 1 to 3 groups independently chosen from cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —[O(CH₂)_{q}]ᵣOH, —OC(═O)R^{k}, —OC(═O)OR^{k}, —OC(═O)NR^{h}Rⁱ, —SR^{k}, —S(═O)ₚR^{k}, —S(═O)ₚNR^{h}Rⁱ, —[O(CH₂)_{q}]ᵣO(C₁-C₄ alkyl), —O—(C₆ aryl or 5- to 8-membered heteroaryl) (optionally substituted with 1 to 3 R^{m} groups), C₃-C₆ carbocyclyl (optionally substituted with 1 to 3 R^{m} groups), C₆ to C₁₀ aryl (optionally substituted with 1 to 3 R^{m} groups), 4- to 10-membered heterocyclyl (optionally substituted with 1 to 3 R^{m} groups), and 5- to 10-membered heteroaryl (optionally substituted with 1 to 3 R^{m} groups) groups;
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₆ alkyl (optionally substituted with 1 to 3 R^{m} groups), —C(═O)R^{k}, —C(═O)OR^{k},
—NR^{h}Rⁱ, —OR^{k}, —S(═O)ₚR^{k}, —S(═O)ₚNR^{h}Rⁱ, and 5- to 10-membered heterocyclyl groups, wherein:
R^{h}, Rⁱ, and R^{j}, for each occurrence, are each independently chosen from hydrogen, C₁-C₆ alkyl (optionally substituted with 1 to 4 R^{m} groups), C₆-C₁₀ aryl, C₃-C₈ carbocyclyl (optionally substituted with 1 to 3 R^{m} groups), 5- to 10-membered heteroaryl (optionally substituted with 1 to 3 R^{m} groups), and 5- to 10-membered heterocyclyl (optionally substituted with 1 to 3 R^{m} groups) groups, wherein:
the C₁-C₆ alkyl of any one of R^{h}, Rⁱ, and R^{j} is optionally substituted with 1 to 3 groups independently chosen from halogen, cyano, —OH, C₁-C₄ alkoxy, —C(═O)NH(C₁-C₄ alkyl), 5- to 10-membered heteroaryl (optionally substituted with 1 to 3 R^{m} groups), and 5- to 10-membered heterocyclyl (optionally substituted with 1 to 3 R^{m} groups) groups;
R^{k}, for each occurrence, is independently chosen from hydrogen, NH₂, (optionally substituted with 1 or 2 groups chosen from C₁-C₃ alkyl), C₁-C₆ alkyl, benzyl, C₆ aryl, C₃-C₆ carbocyclyl, 5- to 10-membered heterocyclyl, and 5- to 10-membered heteroaryl groups, wherein:
the C₁-C₆ alkyl of any one of R^{k} is optionally substituted with 1 to 5 groups independently chosen from halogen, cyano, —NH₂, —OH, C₁-C₄ alkoxy, C₃-C₆ cycloalkyl (optionally substituted with 1 to 3 halogen groups), 5- to 10-membered heterocyclyl (optionally substituted with 1 to 3 —OH groups), 5- to 10-membered aryl (optionally substituted with 1 to 3 groups selected from C₁-C₄ alkyl and halogen), and 5- to-10-membered heteroaryl (optionally substituted with 1 to 3 —OH groups) groups; and
the C₃-C₆ carbocyclyl, benzyl, and C₆ aryl of any one of R^{k} are each optionally substituted with 1 to 3 groups independently chosen from halogen, cyano, oxo, —OH, —C(═O)NH₂, —C(═O)N(CH₃)₂, C₁-C₄ alkyl (optionally substituted by 1 to 3 —OH groups), C₁-C₆ haloalkyl, C₁-C₆ alkoxy, C₃-C₆ cycloalkyl (optionally substituted with 1 to 3 halogen groups), C₆ aryl (optionally substituted with 1 to 3 halogen groups), and 5- to 10-membered heteroaryl (optionally substituted with 1 to 3 halogen groups) groups; and
the 5- to 10-membered heteroaryl and 5- to 10-membered heterocyclyl of any one of R^{k} are each optionally substituted with 1 to 3 groups independently chosen from halogen, oxo, cyano, —C(═O)CH₃, —NH₂, —OH, C₁-C₄ alkyl (optionally substituted by 1 to 3 —OH groups), C₁-C₄ haloalkyl, 5- to 10-membered heterocyclyl, and C₁-C₄ alkoxy groups;
R^{m}, for each occurrence, is independently chosen from halogen, cyano, oxo, —NH₂, C₁-C₆ alkyl, C₁-C₆ alkoxy, —C(═O)R^{k}, —S(═O)ₚR^{k}, —OR^{k}, and 5- to 10-membered heterocyclyl groups, wherein:
the C₁-C₆ alkyl, the C₁-C₆ alkoxy, and the 5- to 10-membered heterocyclyl of any one of R^{m} is optionally substituted with 1 to 3 groups independently chosen from halogen, cyano, —OH, and C₁-C₄ alkoxy groups;
p, for each occurrence, is an integer independently chosen from 1 and 2; and
q and r, for each occurrence, is an integer independently chosen from 0, 1, 2, and 3.

57. The compound, tautomer, deuterated derivative, or pharmaceutically acceptable salt according to any one of claims 1 to 56 wherein R⁶ is chosen from C₁-C₆ alkyl optionally substituted with 1 to 5 groups independently chosen from halogen, cyano, —OH, —NH₂, —NH(C₁-C₄ alkyl), —N(C₁-C₄ alkyl)₂, —C(═O)NH₂, —C(═O)(C₁-C₄ alkyl), —C(═O)OH, —C(═O)O(C₁-C₄ alkyl), —C(═O)NH(C₁-C₄ alkyl), —C(═O)N(C₁-C₄ alkyl)₂, C₁-C₄ alkoxy, C₃-C₆ carbocyclyl, C₆ aryl (which is optionally substituted with 1 to 3 groups independently chosen from halogen and C₁-C₄ haloalkyl groups), —O—(C₆ aryl) (which is optionally substituted with 1 to 3 groups independently chosen from halogen and C₁-C₄ haloalkyl groups), 5- to 10-membered heterocyclyl, and 5- to 10-membered heteroaryl groups.

58. The compound, tautomer, deuterated derivative, or pharmaceutically acceptable salt according to any one of claims 1 to 57, wherein, R⁶ is a substituted C₁-C₆ alkyl chosen from

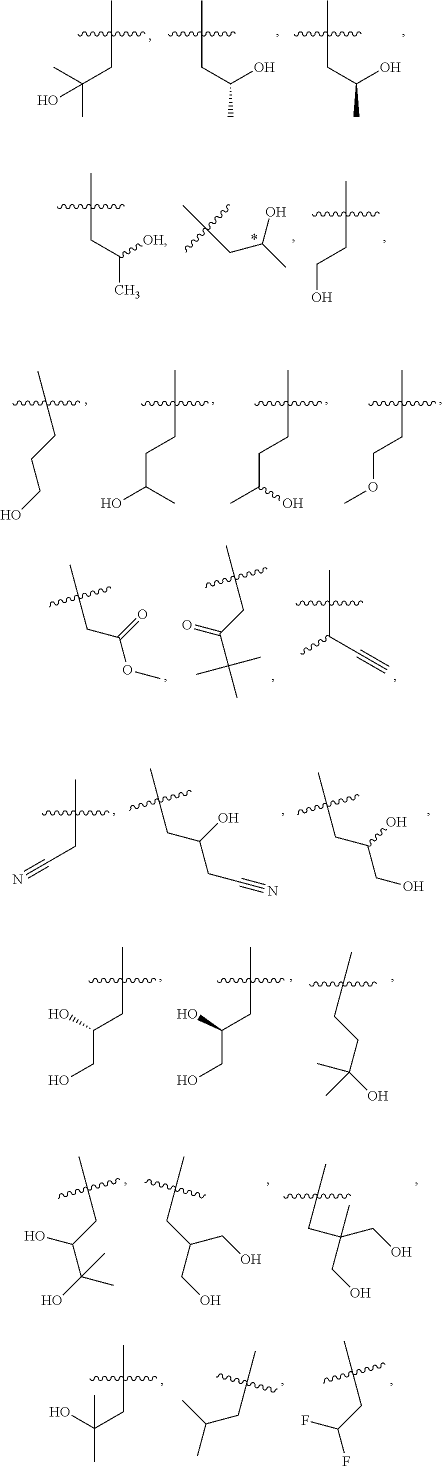

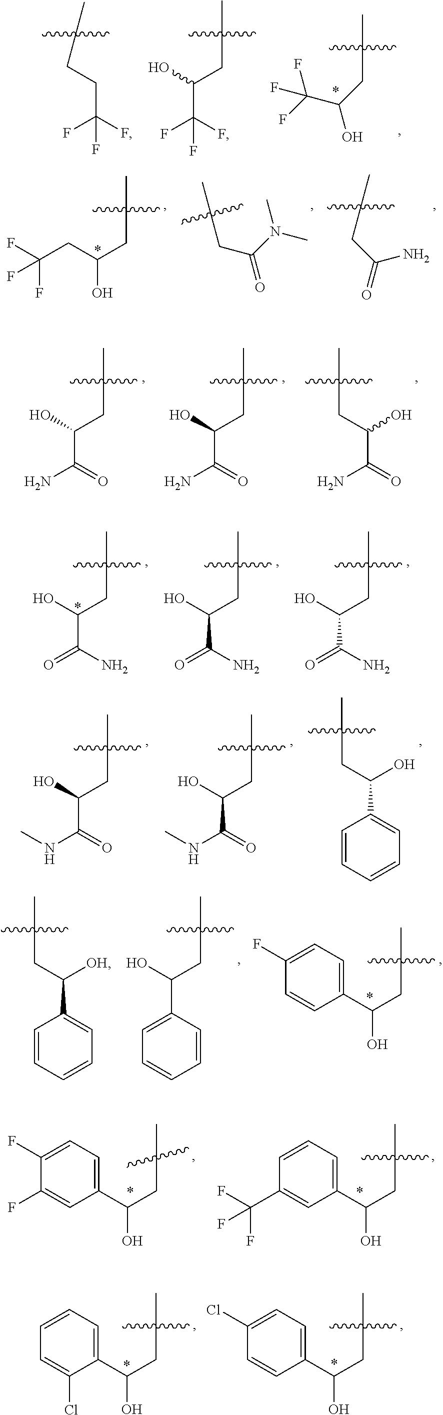

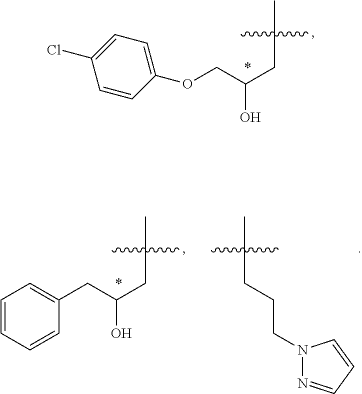 [SMILES: *CC(O)COc1ccc(Cl)cc1 ; "," ; *CC(O)Cc1ccccc1 ; "," ; *CCCn1cccn1 ; "."]

58. The compound, tautomer, deuterated derivative, or pharmaceutically acceptable salt according to any one of claims 1 to 56, wherein R⁶ is chosen from —C(═O)O(C₁-C₄ alkyl).

59. The compound, tautomer, deuterated derivative, or pharmaceutically acceptable salt according to any one of claims 1 to 56, wherein R⁶ is chosen from R⁶ is chosen from 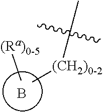
as defined in claim 56.

60. The compound, tautomer, deuterated derivative, or pharmaceutically acceptable salt according to claim 59, wherein Ring B is chosen from C₃-C₁₂ carbocyclyl optionally substituted with 1, 2, 3, 4, or 5 R^{a} groups as defined in claim 56.

61. The compound, tautomer, deuterated derivative, or pharmaceutically acceptable salt according to claim 59, wherein Ring B is chosen from 3- to 12-membered heterocyclyl optionally substituted with 1, 2, 3, 4, or 5 R^{a} groups as defined in claim 56.

62. The compound, tautomer, deuterated derivative, or pharmaceutically acceptable salt according to claim 59, wherein Ring B is chosen from C₆ and C₁₀ aryl optionally substituted with 1, 2, 3, 4, or 5 R^{a} groups as defined in claim 56.

63. The compound, tautomer, deuterated derivative, or pharmaceutically acceptable salt according to claim 59, wherein Ring B is chosen from 5- to 10-membered heteroaryl groups optionally substituted with 1, 2, 3, 4, or 5 R^{a} groups as defined in claim 56.

64. The compound, tautomer, deuterated derivative, or pharmaceutically acceptable salt according to claim 59, wherein Ring B is chosen from: 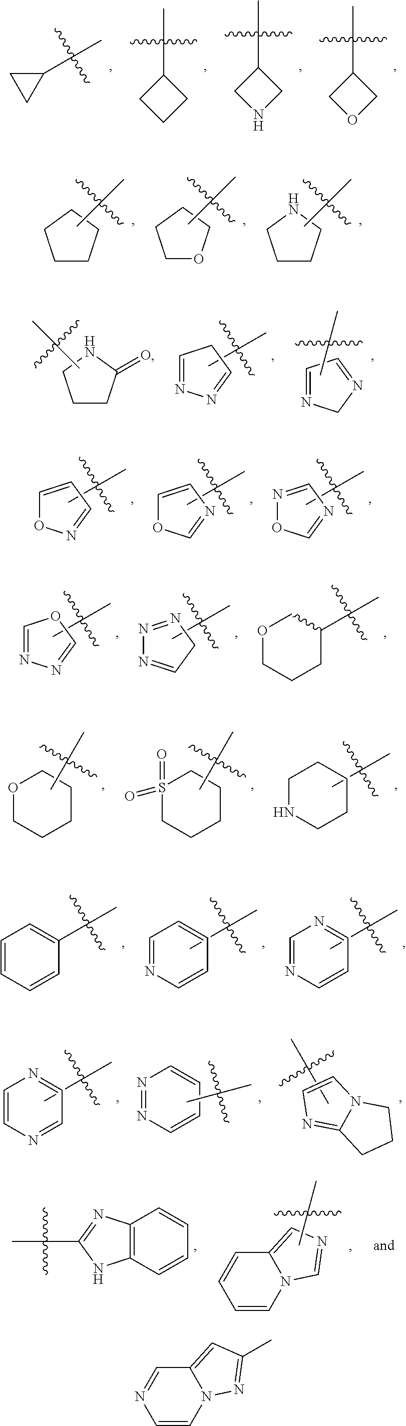
groups optionally substituted with 1, 2, 3, 4, or 5 R^{a} groups as defined in claim 56.

65. The compound, tautomer, deuterated derivative, or pharmaceutically acceptable salt according to claim 64, wherein Ring B is chosen from: 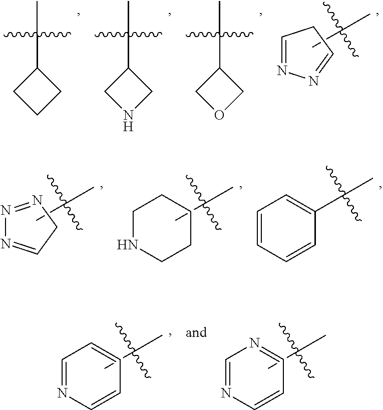
groups optionally substituted with 1, 2, 3, 4, or 5 R^{a} groups as defined in claim 56.

66. The compound, tautomer, deuterated derivative, or pharmaceutically acceptable salt according to claim 56, wherein R⁶ in the is chosen from: 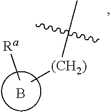
wherein Ring B is a 5-membered heteroaryl, and R^{a} is as defined for Formula I.

67. The compound, tautomer, deuterated derivative, or pharmaceutically acceptable salt according to claim 56, wherein R⁶ is chosen from: 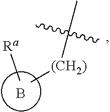
wherein Ring B is a 5-membered heteroaryl, and R^{a} is oxo or is chosen from C₁-C₈ alkyl, C₃-C₁₂ carbocyclyl and C₆ and C₁₀ aryl, each of which may be optionally substituted with 1 to 3 groups chosen from halogen and C₁-C₈ alkyl (wherein the C₁-C₈ alkyl may be optionally substituted with 1 to 3 groups chosen from halogen, —OH, SO₂CH₃, and SO₂NH₂).

68. The compound, tautomer, deuterated derivative, or pharmaceutically acceptable salt according to claim 56, wherein R⁶ is chosen from:

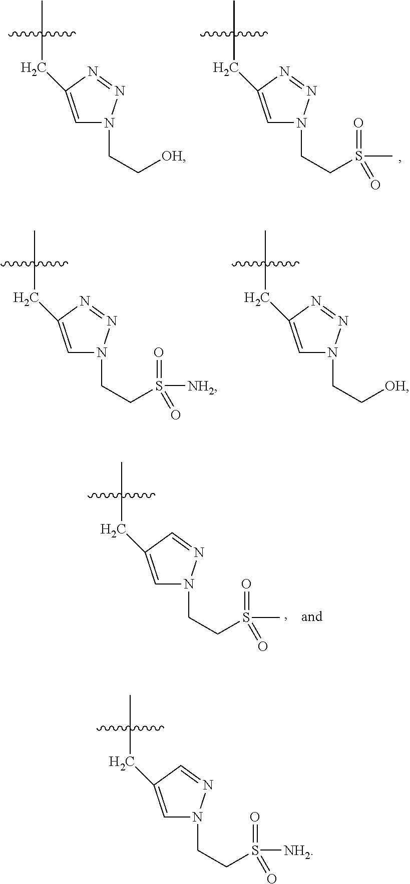 [SMILES: *Cc1cn(CCO)nn1 ; *Cc1cn(CCS(C)(=O)=O)nn1 ; *Cc1cn(CCS(N)(=O)=O)nn1 ; *Cc1cn(CCO)nn1 ; *Cc1cnn(CCS(C)(=O)=O)c1 ; *Cc1cnn(CCS(N)(=O)=O)c1]

69. The compound, tautomer, deuterated derivative, or pharmaceutically acceptable salt according to claim 59, wherein R^{a} is chosen from C₁-C₄ alkyl, halogen, —OH, and C₁-C₄ alkoxy, wherein the C₁-C₄ alkyl of R^{a} is optionally substituted with 1 to 3 polar groups, e.g. sulfones, sulfonamides, and alcohols.

70. The compound, tautomer, deuterated derivative, or pharmaceutically acceptable salt according to claim 59, wherein R^{a} is C₁-C₆ alkyl, optionally substituted as defined for Formula I. In some embodiments, the C₁-C₆ alkyl of R^{a} is optionally substituted with 1 to 3 groups selected from —OH, —SO₂CH₃, C₁-C₃ alkoxy, C(═O)NHCH₃, —SO₂NHCH₂CH₂OH, —SCF₃, —SCH₂C(CH₂)₂OH, —SO₂phenyl, 4-6 membered heterocycles (optionally substituted with 1 to 3 R^{m} groups), 4-6 membered heteroaryls (optionally substituted with 1 to 3 R^{m} groups), cyano, NHC(═O)-4-6 membered heteroaryl.

71. The compound, tautomer, deuterated derivative, or pharmaceutically acceptable salt according to claim 59, wherein R^{a} is chosen from optionally substituted 4-6 membered carobcycles, 4-6 membered heterocycles and 4-6 membered heteroaryls.

72. A compound selected from Compounds 1 to 1183, tautomers thereof, deuterated derivatives of those compounds and tautomers, and pharmaceutically acceptable salts of any of the foregoing, wherein the compound is not Compound 285, Compound 489, Compound 539, Compound 691, Compound 692, Compound 741, Compound 747, Compound 749, Compound 751, Compound 752, Compound 753, Compound 795, Compound 814, or Compound 868.

73. A pharmaceutical composition comprising a compound according to any one of claims 1 to 72.

74. A method of treating a disease mediated by ApoL1, comprising administering a compound according to any one of claims 1 to 72 or a pharmaceutical composition according to claim 73.

75. The method of treating focal segmental glomerulosclerosis (FSGS), comprising administering a compound according to any one of claims 1 to 72 or a pharmaceutical composition according to claim 73.

76. The method of treating non-diabetic kidney disease (NDKD), comprising administering a compound according to any one of claims 1 to 72 or a pharmaceutical composition according to claim 73.

77. The method of treating cancer mediated by ApoL1, comprising administering a compound according to any one of claims 1 to 72 or a pharmaceutical composition according to claim 73.

78. The method of treating cancer according to claim 77, wherein the cancer is pancreatic cancer.

79. The method of treating according to any one of claims 74 to 78, wherein the patient to be treated possesses an APOL1 genetic variants

80. The method of treating according to claim 79, wherein the genetic variant is chosen from G1: S342G:I384M and G2: N388del:Y389del.

81. A method of inhibiting APOL1 activity comprising contacting said APOL1 with at least one compound, tautomer, deuterated derivative, or pharmaceutically acceptable salt according to any one of claims 1 to 72, or a pharmaceutical composition according to claim 73.

82. Use of a compound according to any one of claims 1 to 72 in the manufacture of a medicament for the treatment of an ApoL1 mediated disease.

83. Use of a compound according to any one of claims 1 to 72 in the manufacture of a medicament for the treatment of FSGS.

84. Use of a compound according to any one of claims 1 to 72 in the manufacture of a medicament for the treatment of NDKD.

85. Use of a compound according to any one of claims 1 to 72 in the manufacture of a medicament for the treatment of cancer mediated by ApoL1.

86. Use of a compound according to any one of claims 1 to 72 in the manufacture of a medicament for the treatment of pancreatic cancer mediated by ApoL1.

87. Use of a compound according to any one of claims 1 to 72 in the manufacture of a medicament for inhibiting the activity of ApoL1 in a patient in need thereof.

88. A compound according to any one of claims 1 to 72, or a pharmaceutical composition according to claim 73, for use in inhibiting the activity of ApoL1 in a patient in need thereof.

89. A compound according to any one of claims 1 to 72, or a pharmaceutical composition according to claim 73, for use in treating an ApoL1 mediated disorder.

90. A compound according to any one of claims 1 to 72, or a pharmaceutical composition according to claim 73, for use in treating FSGS.

91. A compound according to any one of claims 1 to 72, or a pharmaceutical composition according to claim 73, for use in treating NDKD.

92. A compound according to any one of claims 1 to 72, or a pharmaceutical composition according to claim 73, for use in treating cancer mediated by ApoL1.

93. A compound according to any one of claims 1 to 72, or a pharmaceutical composition according to claim 73, for use in treating pancreatic cancer mediated by ApoL1.
